(19) United States
(12) Patent Application Publication
(10) Pub. No.: US 2025/0319190 A1
(43) Pub. Date: Oct. 16, 2025

(54) APOE LIPOPROTEIN SYSTEMS

(71) Applicant: Kisbee Therapeutics, Inc., Cambridge, MA (US)

(72) Inventors: Emily Louise RICQ, Cambridge, MA (US); Christopher James GERRY, Cambridge, MA (US)

(21) Appl. No.: 18/865,382

(22) PCT Filed: May 12, 2023
(86) PCT No.: PCT/US2023/022069

§ 371 Date: Nov. 13, 2024

Related U.S. Application Data
(60) Provisional application No. 63/341,537, filed on May 13, 2022.

(51) Int. Cl. A61K 47/42; A61K 47/24; C07K 14/775

(52) U.S. Cl. CPC A61K 47/42; A61K 47/24; C07K 14/775

(57) ABSTRACT

The present disclosure includes systems and compositions based on lipoproteins. The lipoprotein systems may optionally include payloads for delivery to cells, tissues, organs and/or subjects in need thereof. Methods of utilizing the lipoprotein systems are also provided herein.

### BACKGROUND

Lipoproteins are complex molecular assemblies that are key participants in the cascade of intracellular and/or extracellular lipid metabolism. Lipids play key biological roles serving as structural components of cell membranes, energy storehouses and mediators of key signaling events. For example, brain lipids play roles in membrane formation, synaptogenesis, neurogenesis, and impulse conduction. Modulation of lipids has potential utility in the management or prevention of diseases (acute or chronic), syndromes, injuries, and/or to impart a protective effective. Compositions to modulate lipid metabolism, lipid trafficking and/or effectively deliver biologically active lipids are in great need.

Effective in vivo delivery of agents such as small molecules, proteins, peptides, nucleic acids and other biomolecules symbolizes an ongoing clinical challenge. The characteristics of lipoproteins make them excellent candidates with highly attractive properties for exploitation as molecular transporters. The present disclosure provides lipoprotein systems that represent a versatile platform that can be manipulated and tuned for specific applications.

### SUMMARY

The present disclosure provides lipoprotein systems including an apolipoprotein and a lipid, e.g., a glycerophospholipid. Apolipoproteins are known for their ability to bind lipid molecules, as well as traverse the blood-brain barrier. The systems of the present disclosure use this feature of apolipoproteins to design particles, e.g., nanoparticles, optionally bound to a payload, capable of modulating lipid metabolism and delivering payloads directly to the brain. The particles of the present disclosure may be formulated into a variety of shapes, e.g., a disc. The lipoprotein systems of the present disclosure may be used in treating a disease or disorder (e.g., a neurological disease, an autoimmune disease, a cardiovascular disease, a vascular disease, a respiratory disease, a metabolic disease, a gastrointestinal disease, a genetic syndrome, a cancer, or a multisystem disease, among others).

The present disclosure harnesses the unprecedented discovery of the biophysical advantages of lipoprotein systems including apolipoproteins such as apolipoprotein E (APOE) and glycerophospholipids such as phosphatidylglycerols (PG). Inclusion of glycerophospholipids such as PG lipids affords the lipoprotein system of the present disclosure improved polydispersity, thermal stability, and binding affinities over other lipoprotein systems. Further, the present disclosure provides novel peptide sequences for use in such lipoprotein systems. Novel polypeptide sequences include a feature-by-feature assembly of sequences disclosed herein to generate functional polypeptide sequences for use in the present invention. The present disclosure provides lipoprotein systems of varying size, shape, dimensions, lipid composition and/or apolipoprotein content which demonstrate beneficial biophysical properties.

In an aspect, the disclosure provides for a lipoprotein system including: (a) an APOE; and (b) a lipid; wherein the lipid is operably associated with the APOE. In some embodiments, the APOE includes an amino acid sequence that is at least 85% identical to any one of SEQ ID NOs: 267-385. In some embodiments, the APOE includes an amino acid sequence that is at least 95% identical to any one of SEQ ID NOs: 267-385. In some embodiments, the APOE includes an amino acid sequence that is any one of SEQ ID NOs: 267-385. In some embodiments, the APOE includes one or more mutations relative to the amino acid sequence relative to SEQ ID NO: 1 or SEQ ID NO: 268, wherein the one or more mutations are selected from the list of: A6T (SEQ ID NO: 388), A7V (SEQ ID NO: 389), L9P (SEQ ID NO: 390), V101 (SEQ ID NO: 391), T11A (SEQ ID NO: 392), T11S (SEQ ID NO: 393), F12Y (SEQ ID NO: 394), F12L (SEQ ID NO: 395), L13R (SEQ ID NO: 396), A18P (SEQ ID NO: 397), A18T (SEQ ID NO: 398), E21 K (SEQ ID NO: 399), A23V (SEQ ID NO: 400), V24L (SEQ ID NO: 401), T26_E29dup (SEQ ID NO: 402), P28L (SEQ ID NO: 403), E31 K (SEQ ID NO: 404), R33C (SEQ ID NO: 405), R33H (SEQ ID NO: 406), Q35R (SEQ ID NO: 407), E37K (SEQ ID NO: 408), Q39R (SEQ ID NO: 409), G41S (SEQ ID NO: 410), R43del (SEQ ID NO: 411), R43C (SEQ ID NO: 412), R43H (SEQ ID NO: 413), L46P (SEQ ID NO: 415), L48V (SEQ ID NO: 416), R50C (SEQ ID NO: 417), R50S (SEQ ID NO: 418), R50G (SEQ ID NO: 419), R50P (SEQ ID NO: 420), R50H (SEQ ID NO: 421), R50L (SEQ ID NO: 422), R56H (SEQ ID NO: 423), T60A (SEQ ID NO: 424), Q64H (SEQ ID NO: 425), E68del (SEQ ID NO: 426), E68V (SEQ ID NO: 428), E68D (SEQ ID NO: 429), L69Q (SEQ ID NO: 430), L69P (SEQ ID NO: 431), L70F (SEQ ID NO: 432), Q73R (SEQ ID NO: 433), V74A (SEQ ID NO: 434), Q76R (SEQ ID NO: 435), E77K (SEQ ID NO: 436), A80E (SEQ ID NO: 437), M82R (SEQ ID NO: 438), D83E (SEQ ID NO: 439), E84K (SEQ ID NO: 440), M86V (SEQ ID NO: 441), E88Q (SEQ ID NO: 442), L89S (SEQ ID NO: 443), K90E (SEQ ID NO: 444), K90N (SEQ ID NO: 445), Y92C (SEQ ID NO: 446), K93fs (SEQ ID NO: 447), K93T (SEQ ID NO: 448), K93R (SEQ ID NO: 449), K93N (SEQ ID NO: 450), S94* (SEQ ID NO: 451), Q99K (SEQ ID NO: 452), Q99R (SEQ ID NO: 453), P102R (SEQ ID NO: 454), V103L (SEQ ID NO: 455), V103L (SEQ ID NO: 456), A104T (SEQ ID NO: 457), E106Q (SEQ ID NO: 458), R108_A118dup (SEQ ID NO: 459), R108Q (SEQ ID NO: 460), R110fs (SEQ ID NO: 461), R110Q (SEQ ID NO: 461), S112Y (SEQ ID NO: 462), K113E (SEQ ID NO: 463), E114K (SEQ ID NO: 464), L115R (SEQ ID NO: 465), A117V (SEQ ID NO: 466), Q119P (SEQ ID NO: 467), A120S (SEQ ID NO: 468), A120D (SEQ ID NO: 469), R121W (SEQ ID NO: 470), G123R (SEQ ID NO: 471), D128N (SEQ ID NO: 472), V129M (SEQ ID NO: 473), G131C (SEQ ID NO: 474), R132C (SEQ ID NO: 475), R132L (SEQ ID NO: 476), Q135R (SEQ ID NO: 477), Y136C (SEQ ID NO: 478), R137C (SEQ ID NO: 479), R137G (SEQ ID NO: 480), R137H (SEQ ID NO: 481), G138C (SEQ ID NO: 482), V140L (SEQ ID NO: 483), V140M (SEQ ID NO: 484), L1441 (SEQ ID NO: 485), G145R (SEQ ID NO: 486), G145S (SEQ ID NO: 487), G145D (SEQ ID NO: 488), G145A (SEQ ID NO: 489), Q146* (SEQ ID NO: 490), L151M (SEQ ID NO: 491), R152W (SEQ ID NO: 492), R152Q (SEQ ID NO: 493), V153M (SEQ ID NO: 494), R154S (SEQ ID NO: 330), R154fs (SEQ ID NO: 495), R154C (SEQ ID NO: 496), R154L (SEQ ID NO: 497), S157F (SEQ ID NO: 498), L159P (SEQ ID NO: 499), R160G (SEQ ID NO: 500), 163C (SEQ ID NO: 501), R163H (SEQ ID NO: 502), K164Q (SEQ ID NO: 503), L167del (SEQ ID NO: 504), R168H (SEQ ID NO: 505), D169Y (SEQ ID NO: 506), D172N (SEQ ID NO: 507), Q174K (SEQ ID NO: 508), V179A (SEQ ID NO: 509), Y180H (SEQ ID NO: 510), Q181E (SEQ ID NO: 511), Q181H (SEQ ID NO: 512), A184S (SEQ ID NO: 513), G187A (SEQ ID NO: 514), G187V (SEQ ID NO: 515), G191_R198dup (SEQ ID NO: 516), E189K (SEQ ID NO: 517), R190C (SEQ ID NO: 518), G191S (SEQ ID NO: 519), S193R (SEQ ID NO: 520), R196C (SEQ ID NO: 521), R198C (SEQ ID NO: 522), G200R (SEQ ID NO: 523), R207S (SEQ ID NO: 524), R207C (SEQ ID NO: 525), R207H (SEQ ID NO: 526), R207P (SEQ ID NO: 527), V208L (SEQ ID NO: 528), V208M (SEQ ID NO: 529), V213G (SEQ ID NO: 530), G214S (SEQ ID NO: 531), G218R (SEQ ID NO: 532), G218S (SEQ ID NO: 533), Q219E (SEQ ID NO: 534), P220L (SEQ ID NO: 535), L221 P (SEQ ID NO: 536), Q222* (SEQ ID NO: 537), Q222K (SEQ ID NO: 538), Q222R (SEQ ID NO: 539), R224P (SEQ ID NO: 540), R224L (SEQ ID NO: 541), A225T (SEQ ID NO: 542), A227S (SEQ ID NO: 543), W228* (SEQ ID NO: 544), G229R (SEQ ID NO: 545), G229V (SEQ ID NO: 546), E230K (SEQ ID NO: 547), R231Q (SEQ ID NO: 548), A234T (SEQ ID NO: 549), A234V (SEQ ID NO: 550), R235W (SEQ ID NO: 551), R235Q (SEQ ID NO: 552), E238del (SEQ ID NO: 553), E238D (SEQ ID NO: 554), M2391 (SEQ ID NO: 555), G240R (SEQ ID NO: 556), S241R (SEQ ID NO: 557), T243fs (SEQ ID NO: 558), R242W (SEQ ID NO: 559), R242G (SEQ ID NO: 560), R242P (SEQ ID NO: 561), T243N (SEQ ID NO: 562), R246S (SEQ ID NO: 563), R246C (SEQ ID NO: 564), R246P (SEQ ID NO: 565), D248E (SEQ ID NO: 566), E249K (SEQ ID NO: 567), E249Q (SEQ ID NO: 568), E256V (SEQ ID NO: 569), R258H (SEQ ID NO: 570), A259P (SEQ ID NO: 571), E262K (SEQ ID NO: 572), E263K (SEQ ID NO: 573), Q267R (SEQ ID NO: 574), V254E (SEQ ID NO: 331), R269fs (SEQ ID NO: 575), R269C (SEQ ID NO: 576), R269G (SEQ ID NO: 577), E273G (SEQ ID NO: 578), A274G (SEQ ID NO: 579), Q276* (SEQ ID NO: 580), A277V (SEQ ID NO: 581), R278C (SEQ ID NO: 582), L279fs (SEQ ID NO: 583), W282C (SEQ ID NO: 584), E284G (SEQ ID NO: 585), E284D (SEQ ID NO: 586), L286M (SEQ ID NO: 587), V287M (SEQ ID NO: 588), D289A (SEQ ID NO: 589), R292C (SEQ ID NO: 590), R292H (SEQ ID NO: 591), W294C (SEQ ID NO: 592), A295T (SEQ ID NO: 593), G296R (SEQ ID NO: 594), G296W (SEQ ID NO: 595), L297P (SEQ ID NO: 596), E299Q (SEQ ID NO: 597), K300R (SEQ ID NO: 598), K300N (SEQ ID NO: 599), V301L (SEQ ID NO: 600), A303S (SEQ ID NO: 601), V305M (SEQ ID NO: 602), T3071 (SEQ ID NO: 603), A309T (SEQ ID NO: 604), P311A (SEQ ID NO: 605), S314R (SEQ ID NO: 606), *318ext (SEQ ID NO: 607), W38* (SEQ ID NO: 608), E98fs (SEQ ID NO: 609), A117T (SEQ ID NO: 610), L122M (SEQ ID NO: 611), Q141R (SEQ ID NO: 612), R160C (SEQ ID NO: 613), R163P (SEQ ID NO: 614), K164E (SEQ ID NO: 615), R165P (SEQ ID NO: 616), A170P (SEQ ID NO: 617), G183A (SEQ ID NO: 618), and combinations thereof. In some embodiments, the one or more mutations include R154S. In some embodiments, the one or more mutations include V254E. In some embodiments, the APOE includes any one of SEQ ID NOs: 275-277.

In some embodiments, the APOE comprises one or more sequence regions selected from a signal sequence, an N-terminus region, a structured sequence region, a hinge region, a linker region, and a C-terminus region. In some embodiments, the APOE comprises more than one structured sequence region. In some embodiments, each structured sequence region is selected from an alpha helix sequence region, a beta confirmation sequence region, and a beta turn sequence region.

In some embodiments, the APOE includes one or more chemical modifications. In some embodiments, the one or more chemical modifications include one or more post-translational modifications (PTM). In some embodiments, the one or more post translational modifications are selected from oxidation, carbamylation, oxi-carbamylation, acetylation, phosphorylation, ubiqutination, biotinylation, carboxylation, deamidation, deamination, deacetylation, dihydroxylation, dephosphorylation, formylation, gamma-carboxyglutamation, glutathionylation, glycation, hydroxylation, methylation, nitration, sumoylation, N- or O-transglutamination, glycosylation and farnesylation.

In some embodiments, the APOE includes an oxidatively coupled dimer, the dimer including two APOE monomers. In some embodiments, the APOE monomer includes an amino acid sequence at least 85% identical to any one of SEQ ID NOs: 267-385. In some embodiments, the APOE monomer includes an amino acid sequence at least 95% identical to any one of SEQ ID NOs: 267-385. In some embodiments, the APOE monomer is any one of SEQ ID NOs: 267-385. In some embodiments, the monomer is any one of SEQ ID NOs: 268, 271, 275-277, or 294.

In some embodiments, the lipid includes the formula (I):

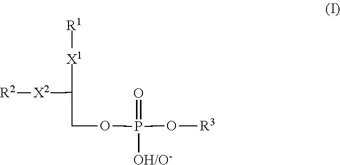

wherein:
X¹ and X² are independently N, O, or absent;
R¹ and R² are independently optionally substituted acyl, optionally substituted alkyl, optionally substituted alkenyl, or absent; and
R³ is selected from:

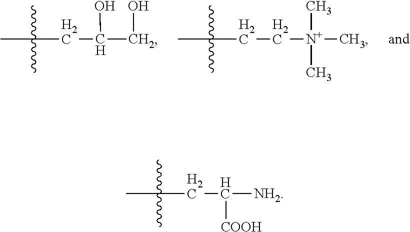

In some embodiments, the lipid is a glycerophospholipid. In some embodiments, the lipid is a lysolipid.

In some embodiments, the lipid is a phosphatidylglycerol. In some embodiments, the phosphatidylglycerol is 1-palmitoyl-2-oleoyl-sn-glycero-3-(phospho-rac-(1-glycerol)) (POPG), 1,2-dimyristolyl-sn-glycero-3-phosphoglycerol (DMPG), or 1,2-dioleoyl-sn-glycero-3-phosphoglycerol (DOPG).

In some embodiments, the lipid is a phosphatidylcholine. In some embodiments, the phosphatidylcholine is 1-palmitoyl-2-oleoyl-sn-glycero-3-phosphocholine (POPC), 1,2-dimyristoyl-sn-glycero-3-phosphocholine (DMPC), 1,2-dipalmitoyl-sn-glycero-3-phosphocholine (DPPC), 1,2-dioleoyl-sn-gycero-3-phosphocholine (DOPC), or N-oleoyl-D-erythro-sphingosylphosphorylcholine (18:1 SM).

In some embodiments, the lipid is a phosphatidylserine. In some embodiments, the phosphatidylserine is 1,2-dimyristoyl-sn-glycero-3-phospho-L-serine (DMPS), 1-palmitoyl-2-oleoyl-sn-glycero-3-phospho-L-serine (POPS), or 1,2-dioleoyl-sn-glycero-3-phospho-L-serine (DOPS).

In some embodiments, the lipid includes at least 2 unique lipids. In some embodiments, the at least two unique lipids include a first lipid and a second lipid. In some embodiments, the molar ratio of the first lipid to the second lipid is from about 1:100 of the first lipid to the second lipid, to about 100:1 of the first lipid to the second lipid. In some embodiments, the molar ratio of the first lipid to the second lipid is about 1:1 of the first lipid to the second lipid. In some embodiments, the first lipid is POPC and the second lipid is POPS.

In some embodiments, the APOE and the lipid are in a molar ratio of from 1:10000 APOE to lipid, to 1:1 APOE to lipid. In some embodiments, the APOE and the lipid are in a molar ratio of from 1:500 APOE to lipid, to 1:30 APOE to lipid. In some embodiments, the APOE and the lipid are in a molar ratio of from 1:200 APOE to lipid, to 1:50 APOE to lipid. In some embodiments, the APOE and the lipid are in a molar ratio of 1:100 APOE to lipid.

In some embodiments, the system has a discoidal, a spherical, a cylindrical, a lamellar, or an ellipsoidal form. In some embodiments, the system has a discoidal form. In some embodiments, the system has a diameter of from 1 nm to 100 nm. In some embodiments, the system has a percent polydispersity of at most 25%. In some embodiments, the system has a percent polydispersity of at most 20%. In some embodiments, the system has a percent polydispersity of at most 15%. In some embodiments, the system has a percent polydispersity of at most 10%. In some embodiments, the system has a percent polydispersity of at most 5%.

In some embodiments, the lipoprotein system further includes a payload. In some embodiments, the payload is selected from the group consisting of a peptide payload, a protein payload, a nucleic acid payload, a lipid payload, a lipid derivative payload, a carbohydrate payload, a glycolipid payload, a metabolite payload, a metabolite derivative payload, and a small molecule payload.

In some embodiments, the payload is a small molecule payload. In some embodiments, the small molecule is selected from an antioxidant, an antineoplastic agent, an analgesic, an anesthetic, an antibacterial, an anticonvulsant, an antidementia agent, an antidepressant, an antiemetic, an antifungal, an antigout agent, an anti-inflammatory agent, an antimigraine agent, an antimyasthenic agent, an antimycobacterial agent, an antiparasitic, an antiparkinson agent, an antipsychotic, an antispasticity agent, an antiviral, an anxiolytic, a bipolar agent, a blood glucose regulator, a blood product, a blood modifier, a blood volume expander, a chemotherapeutic agent, a cardiovascular agent, a central nervous system agent, a dental oral agent, a dermatological agent, an electrolyte, an enzyme replacement, an enzyme modifier, a gastrointestinal agent, a genitourinary agent, an immunological agent, an inflammatory bowel disease agent, a metabolic bone disease agent, an ophthalmic agent, an otic agent, a respiratory tract agent, a sedative, a hypnotic, and a skeletal muscle relaxant.

In some embodiments, the payload is a nucleic acid. In some embodiments, the nucleic acid is RNA, DNA or cDNA. In some embodiments, the RNA is mRNA, siRNA, microRNA, interference RNA, replicon mRNA, guide RNA, short hairpin RNA (shRNA), piwi-interacting RNA (piRNA), a non-coding RNA (ncRNA), a long non-coding RNA (lncRNA), a double-stranded RNA (dsRNA), a single-stranded RNA, or a circular RNA (circRNA).

In some embodiments the nucleic acid is an antisense oligonucleoside (ASO). In some embodiments, the antisense oligonucleotide may be modified (e.g., with a modification at the ribose portion of the nucleoside or at the internucleoside linkage).

In some embodiments, the lipoprotein system further includes a lipoprotein system modifier. In some embodiments, the lipoprotein system modifier is a targeting agent, a regulatory agent, a solubilizing agent, a stabilizing agent, or a detection agent.

In a further aspect, the disclosure provides for methods of treating or preventing a disease, or disorder in a subject, the method including administering a lipoprotein system described herein. In some embodiments, the disease, injury, or disorder is a neurological disease, an autoimmune disease, a cardiovascular disease, a vascular disease, a respiratory disease, a metabolic disease, a gastrointestinal disease, a genetic syndrome, a cancer, or a multisystem disease. In some embodiments, the disease, injury, or disorder is a neurological disease. In some embodiments, the disease, injury, or disorder is selected from one or more of: Degenerative Cervical Myelopathy, Niemann-Pick disease, amyotrophic lateral sclerosis (ALS), multiple sclerosis, Parkinson's disease, Alzheimer's disease, Huntington's disease, Smith-Lemli Opitz syndrome (SLOS), multiple system atrophy, prion disease, impaired cognitive function, or dementia. In some embodiments, the neurological disease is Niemann-Pick disease. In some embodiments, the neurological disease is Alzheimer's disease. In some embodiments, the neurological disease is severe acute respiratory syndrome coronavirus 2 (SARS CoV-2) infection, or a neurological disease resulting from a SARS CoV-2 infection.

### Definitions

As used herein, the terms “apolipoprotein” and its abbreviation “APO” refers to a protein that binds to and/or is capable of interacting with a lipid.

As used herein, the term “apolipoprotein E” or the abbreviation “APOE” refers to any apolipoprotein E, including fragments and variants thereof.

As used herein, the term “apolipoprotein variant” refers to a form or a version of the apolipoprotein polypeptide or a polynucleotide that differs in some respect from other forms of the apolipoprotein polypeptide or polynucleotide or from an apolipoprotein polypeptide or a polynucleotide reference standard. Such apolipoprotein polypeptide or polynucleotide reference standards may be a parental apolipoprotein polypeptide or polynucleotide or a starting apolipoprotein polypeptide or a polynucleotide having differences in structure, sequence, or function which distinguish the apolipoprotein variant from starting or reference standard. As used herein, apolipoprotein variants also include apolipoprotein mutations, which may include amino acid substitution, deletion, insertion, and amino acid sequence alteration (e.g., reverse oriented sequence, scrambled sequence, multidomain sequence, switched-domain sequence). As used herein, apolipoprotein variants also include fusion polypeptides.

As used herein, the term “alpha helix sequence region” refers to a type of a structure in which the polypeptide backbone is tightly wound around an imaginary axis drawn longitudinally through the middle and the R groups of the amino acids protrude outward from the backbone. In this confirmation, every backbone N-H group forms a hydrogen bond with the backbone C═O group of the amino acid located four residues earlier along the protein sequence.

As used herein, the term “beta confirmation sequence region” refers to a type of structure in which the backbone of the polypeptide is extended into a zigzag pattern rather than a helical structure. In some embodiments, the zigzag polypeptide chains can be arranged side by side to form a beta sheet.

As used herein, the term “beta turn sequence region” refers to a type of structure which connects the ends of two adjacent segments of a beta sheet.

As used herein, the term “derivative” refers to a form or a version of a compound that differs from a parent compound by at least one atom or a group.

As used herein, the term “fatty acid” refers to carboxylic acids with long-chain hydrocarbon side groups.

As used herein, the term “fragment” in the context of a polypeptide refers to a continuous stretch of amino acids in the linear sequence of a polypeptide.

As used herein, the term “isolated” means separated from constituents, cellular and otherwise, in which the polynucleotide, polypeptide, protein, or fragments thereof, are normally associated with in nature. For example, with respect to a polypeptide, an isolated polypeptide is one that is separated from the amino and carboxyl ends with which it is normally associated in the naturally occurring sequence. As is apparent to those of skill in the art, a non-naturally occurring polynucleotide, polypeptide, protein, or fragments thereof, does not require “isolation” to distinguish it from its naturally occurring counterpart. In addition, a “concentrated,” “separated,” or “diluted” polynucleotide, polypeptide, protein, or fragments thereof, is distinguishable from its naturally occurring counterpart in that the concentration or number of molecules per volume is greater than “concentrated” or less than “separated” or “diluted” than that of its naturally occurring counterpart. A polynucleotide, polypeptide, protein, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Thus, a non-naturally occurring polypeptide is provided as a separate embodiment from the isolated naturally occurring polypeptide. A protein produced in a bacterial cell is provided as a separate embodiment from the naturally occurring protein isolated from a eukaryotic cell in which it is produced in nature.

As used herein, the term “lipoprotein system” refers to a biochemical assembly that includes one or more apolipoproteins and one or more lipids.

As used herein, the term “lipoprotein system modifier” refers to any macro molecule or micro molecule that can alter/tune the location, structure, function, solubility, detection, synthesis, assembly, and/or degradation of a lipoprotein system in vitro, ex vivo or in vivo.

As used herein, the term “lipid” refers to molecules of biological origin that are soluble in organic solvents (e.g., chloroform) but show little to no solubility in water.

As used herein, the term “nucleic acid” refers to RNA or DNA molecules consisting of a chain of ribonucleotides or deoxyribonucleotides, respectfully. Further, as used herein, “nucleic acid” includes any compound or substance that includes a polymer of nucleotides e.g., linked nucleosides. Such polymers may be referred to as polynucleotides.

As used herein, the term “nucleoside” refers to a molecule made up of a heterocyclic base and its sugar.

As used herein, the term “nucleotide” refers to a nucleoside having a phosphate group, or a variant thereof, on its 3′ or 5′ sugar hydroxyl group. Phosphate group variants include, but are not limited to, saturated alkyl phosphonates, unsaturated alkenyl phosphonates, phosphothioates, and phosphoramidites.

As used herein, the phrase “operably linked” refers to a functional connection between two or more molecules, lipids, polypeptides, entities, moieties or the like.

As used herein, the term “portion” in the context of a polypeptide refers to a segment derived from or one or more fragments of a polypeptide.

The term “payload,” as used herein, refers to any molecule that is associated with the lipoprotein system for delivery to a cell, tissue, subject or a biological system.

“Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, the term “protective mutation” is a mutation in a polypeptide or a polynucleotide relative to a parental polypeptide or a parental polynucleotide whose presence may prevent, protect, or shield the polynucleotide or the polypeptide or the cell, organ, system or organism containing the polypeptide or the polynucleotide against one or more cellular outcomes or a state associated with a disease or a disorder e.g., neurodegeneration. As a non-limiting example, a protective mutation may prevent or reduce the occurrence of aggregation of the polypeptide containing the protective mutation, reduce binding to heparin, inflammation, tauopathy disease progression and/or neurodegeneration.

As used herein, the term “region” refers to a zone or general area. In some embodiments, when referring to a protein or a polypeptide, a region may include a linear sequence of amino acids along the protein or may include a three dimensional area, an epitope and/or a cluster of epitopes. In some embodiments, regions include terminal regions. As used herein, the term “terminal region” refers to regions located at the ends or termini of a given agent. When referring to proteins, terminal regions may include N- and/or C-termini. N-termini refer to the end of a protein comprising an amino acid with a free amino group. C-termini refer to the end of a protein comprising an amino acid with a free carboxyl group. N- and/or C-terminal regions may therefore include the N- and/or C-termini as well as surrounding amino acids. In some embodiments, N-terminal regions may include any length of amino acids that includes the N-terminus but does not include the C-terminus. In some embodiments, C-terminal regions may include any length of amino acids, which include the C-terminus, but do not include the N-terminus.

As used herein, “structured sequence region” refers to a region of a polypeptide containing one or more folding patterns in its backbone formed by the stable arrangement of amino acid residues of the polypeptide. The folding pattern may be an alpha helix, a beta confirmation, or a beta turn.

As used herein, the phrase “signal sequences” refers to a sequence which can direct the transport or localization of a polypeptide.

As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, a “variant” is a form or a version of polypeptide or a polynucleotide that differs in some respect from other forms of the same polypeptide or polynucleotide or from a polypeptide or a polynucleotide reference standard. Such polypeptide or polynucleotide reference standards may be a parental polypeptide or polynucleotide or a starting polypeptide or a polynucleotide having differences in structure, sequence, or function which distinguish the variant from starting or reference standard.

As used herein, the term “unique polypeptide” is meant to describe that a polypeptide that has a distinctive feature or a combination of distinctive features that are not present in another polypeptide. The distinctive feature may refer to the sequence of amino acids (natural and/or non-natural) in the polypeptide, the secondary structures, post translational modifications, chemical modifications, and/or functional features.

As used herein, the term “unique lipid” is meant to describe that a lipid that has a distinctive feature or a combination of distinctive features that are not present in lipid. The distinctive feature may refer to the primary structure of the lipid, the confirmation of the lipid, functional groups of the lipid, modifications to the structure of the lipid and/or charge of the lipids.

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

### DETAILED DESCRIPTION

The present disclosure provides lipoprotein systems including an apolipoprotein and a lipid, e.g., a glycerophospholipid. Apolipoproteins are known for their ability to bind lipid molecules, as well as traverse the blood-brain barrier. The systems of the present disclosure use this feature of apolipoproteins to design particles, e.g., nanoparticles, optionally bound to a payload, capable of modulating lipid metabolism and delivering payloads directly to the brain. The particles of the present disclosure may be formulated into a variety of shapes, e.g., a disc. The lipoprotein systems of the present disclosure may be used in treating a disease or disorder (e.g., a neurological disease, an autoimmune disease, a cardiovascular disease, a vascular disease, a respiratory disease, a metabolic disease, a gastrointestinal disease, a genetic syndrome, a cancer, or a multisystem disease, among others).

The present disclosure harnesses the unprecedented discovery of the biophysical advantages of lipoprotein systems including apolipoproteins such as apolipoprotein E (APOE) and glycerophospholipids such as phosphatidylglycerols (PG). Inclusion of glycerophospholipids such as PG lipids affords the lipoprotein system of the present disclosure improved polydispersity, thermal stability, and binding affinities over other lipoprotein systems. The present disclosure also provides novel peptide sequences for use in such lipoprotein systems. Novel polypeptide sequences are generated via feature-by-feature assembly of sequences disclosed herein to generate functional polypeptide sequences which may be formulated into lipoprotein systems. The present disclosure provides lipoprotein systems of varying size, shape, dimensions, lipid composition and/or apolipoprotein content which demonstrate beneficial biophysical properties.

Arrangement and/or positioning of components within the system may be optimized to achieve a particular form or function. In some embodiments, the lipids may be arranged as a bilayer whereas the polypeptides of the disclosure may encircle or coat the lipids. In some embodiments, one or more lipids may be present as a core, surrounded by monolayer of lipids operably associated with the polypeptides of the disclosure. In some embodiments, the lipoprotein system may be organized in concentric spherical layers.

In some embodiments, the lipoprotein systems may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 300, 400, 500, 600, 700, 800, 900, 1000 or more polypeptides. In some embodiments, the lipoprotein systems may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 300, 400, 500, 600, 700, 800, 900, 1000 or more lipids. In some embodiments, the lipoprotein systems may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 300, 400, 500, 600, 700, 800, 900, 1000 or more payloads.

The lipoprotein systems of the present disclosure may be formatted into particular form. In some embodiments, the identity of the lipids in the lipoprotein system may be altered to adjust the shape or form of the system. In some embodiments, the lipoprotein systems may have discoidal, spherical, cylindrical, lamellar, oblate or ellipsoidal form. In a preferred embodiment, the lipoprotein systems have a discoidal shape.

The amounts and ratios of lipoprotein system components may be varied by any amount dependent on the desired payload, form, function, structure, or any combination thereof. In some embodiments, the amount of each component may be expressed as the relative ratio of each component based on the number of molecules (molar ratio). In some embodiments, the lipoprotein system may include polypeptide and the lipid in a particular molar ratio. When more than one lipid or polypeptide are present in a system, the ratio reflects the combined polypeptide to the lipids, or the ratio of the polypeptide to the combined lipids or the ratio of the combined polypeptide to the combined lipids as the case may be. Non-limiting examples of polypeptide to lipid molar ratio include 1:30, 1:60, 1:90, 1:120, 1:150, 1:180, 1:210, 1:1, 1:2, 1:3, 1:4, 1:5, 1:6, 1:7, 1:8, 1:9, 1:10, 1:20, 1:40, 1:50, 1:70, 1:80, 1:100, 1:110, 1:130, 1:140, 1:160, 1:170, 1:190, 1:200, 1:220, 1:230, 1:240, 1:250, 1:300, 1:400, 1:500, 1:600, 1:700, 1:800, 1:900, 1:1000, 1:5000, 1:10000, 2:1, 2:3, 2:5, 2:7, 2:9, 3:1, 3:2, 3:4, 3:5, 3:7, 3:8, 3:10, 3:20, 3:40, 3:50, 3:70, 3:80, 3:100, 3:110, 3:130, 3:140, 3:160, 3:170, 3:190, 3:200, 3:220, 3:230, 3:250, 3:400, 3:500, 3:700, 3:800, 3:1000, 3:5000, 3:10000, 1:1, 2:1, 3:1, 4:1, 5:1, 6:1, 7:1, 8:1, 9:1, 5:2, 7:2, 9:2, 1:3, 2:3, 4:3, 5:3, 7:3, 8:3, 10:1, 20:1, 30:1, 40:1, 50:1, 60:1, 70:1, 80:1, 90:1, 10:3, 20:3, 40:3, 50:3, 70:3, 80:3, 100:1, 110:1, 120:1, 130:1, 140:1, 150:1, 160:1, 170:1, 180:1, 190:1, 200:1, 210:1, 220:1, 230:1, 240:1, 250:1, 300:1, 400:1, 500:1, 600:1, 700:1, 800:1, 900:1, 100:3, 110:3, 130:3, 140:3, 160:3, 170:3, 190:3, 200:3, 220:3, 230:3, 250:3, 400:3, 500:3, 700:3, 800:3, 1000:1, 5000:1, 1000:3, 5000:3, 10000:1, and/or 10000:3 mol/mol.

In some embodiments, the polypeptide to lipid ratio may be designed to mirror the ratio of apolipoproteins to lipids found in naturally occurring high density lipoprotein particles (HDL). In some embodiments, the lipoprotein systems of the disclosure may include one or more bilayers of lipids. In some embodiments, the lipoprotein systems of the disclosure may be unilamellar, or multilamellar vesicles. Unilamellar vesicles may be small unilamellar vesicles (SUV) with diameters of from about 20 to about 100 nm, large unilamellar vesicles (LUV) with diameters of from about 100 to about 1000 nm, or giant unilamellar vesicles (GUV) with diameters >1000 nm). In some embodiments, the multilamellar vesicles (MLV), may have diameters of >500 nm, in which concentric bilayers form a multilayer structure.

### Polypeptides

Polypeptides are a polymer of amino acids residues linked together, most often by peptide bonds. As used herein the term encompasses proteins, polypeptides, or peptides of any size, structure or function. Polypeptides may include natural, unnatural amino acids or a combination thereof. Polypeptides may include gene products, naturally occurring polypeptides, synthetic polypeptides, homologs, orthologs, analogs or other equivalents thereof.

A polypeptide may be a single molecule or may be a multi-molecular complex such as a dimer, trimer or tetramer. They may also include single chain or multichain polypeptides and may be associated or linked. The term polypeptide may also apply to amino acid polymers in which one or more amino acid residues are an artificial chemical analogue of a corresponding naturally occurring amino acid. When the polypeptide is a multimolecular complex, it may include at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 100, 200, 300, 400, 500, 600, 700, 800, 900, 1000 or more molecules. The molecules may be derived from the same parent protein or from different parent proteins.

Polypeptides of the disclosure may be or may be derived from a parent protein. Any naturally occurring protein may be a parent protein for the polypeptides of the disclosure. In some embodiments, the polypeptide variant may be derived from or may be a variant of a parent protein. The parent protein may be a mammalian protein. In some embodiments, the parent protein may be from a mammal such as, but not limited to, human, mouse, rat, dog, pig, goat, llama, or sheep.

In some embodiments, the polypeptides of the disclosure may be or may be derived from apolipoproteins.

In some embodiments, polypeptide sequence of the disclosure may be tuned to optimize one or more features, including, but not limited to, polypeptide expression in vitro or in vivo, cholesterol efflux, thermal stability, half-life in biological fluids (e.g., cerebrospinal fluid, plasma), delivery of payload, and/or blood brain barrier penetration. In some embodiments, the polypeptide sequence of the disclosure may be tuned to increase polypeptide expression in vitro or in vivo, increased cholesterol efflux, increased thermal stability, increased half-life in biological fluids (e.g., cerebrospinal fluid, plasma), enhanced delivery of payload, and/or improved blood brain barrier penetration. In some embodiments, the polypeptide sequence of the disclosure may be tuned to decrease expression in vitro or in vivo, reduce cholesterol efflux, reduce thermal stability, decrease half-life in biological fluids (e.g., cerebrospinal fluid, plasma), delay delivery of payload, and/or decrease blood brain barrier penetration.

Polypeptides of the disclosure may include one or more amino acids which may mimic an activated sequence. For example, glutamate may serve as a mimic for phospho-threonine and/or phospho-serine. Alternatively, mimics may result in deactivation or in an inactivated product containing the mimic e.g., phenylalanine may act as an inactivating substitution for tyrosine or alanine may act as an inactivating substitution for serine.

Polypeptides of the disclosure may include one or more post translational modifications (PTMs). Modifications may occur in vitro, in vivo and/or intracellularly after administration of the lipoprotein systems to a subject or upon of a polynucleotide encoding the polypeptides of the disclosure. Post translational modifications (PTMs) of the present disclosure include, but are not limited to, oxidation, carbamylation, oxi-carbamylation, acetylation, phosphorylation, ubiqutination, biotinylation, carboxylation, deamidation, deamination, deacetylation, dihydroxylation, dephosphorylation, formylation, gamma-carboxyglutamation, glutathionylation, glycation, hydroxylation, methylation, nitration, sumoylation, N- or O-transglutamination, glycosylation and farnesylationb. In some embodiments, the glycosylation may be N- or O-glycosylation. In some embodiments, the glycosylation may be O-glycosylation. As a non-limiting example, the O-glycosylation may be sialylation. Polypeptides may include 1, 2, 3, 4, 5, 6, 7, 8, 9, 10 or more PTMS. In some embodiments, PTMs may be present in one or more regions of the polypeptide including the structured sequence region, N terminal sequence region, C terminal sequence region or a combination thereof.

Polypeptides may be reversibly or irreversibly chemically crosslinked or circularized by natural (e.g., disulfide bonding, native chemical ligation) or non-natural bonds (e.g., bonds resulting from “click chemistry,” olefin metathesis or dimerization with a bifunctional probe). In some embodiments, polypeptides of the disclosure may be cross linked which involves chemically joining two or more molecules by a covalent bond. Crosslinking may be within regions or portions of one polypeptide or between two or more polypeptides. Attachments between two groups on a single polypeptide result in intramolecular crosslinks that stabilize the polypeptide tertiary or quaternary structure. Attachments between groups on two different polypeptides result in intermolecular crosslinks that stabilize polypeptide-polypeptide interaction. Crosslinking may be achieved using a homobifunctional crosslinking reagent, a heterobifunctional crosslinking agent or a photoreactive crosslinking agent. Non-limiting examples of homobifunctional crosslinking agents include, disuccinimidyl suberate (DSS), disuccinimidyl tartrate (DST), dithiobissuccinimidylpropionate (DSP), bismaleimidoethane (BMOE), 1,4-bismaleimidobutane (BMB), 1,8-bismaleimido-diethyleneglycol (BMB-PEG2), 1,3-dichloroacetone, or DTME. Non-limiting examples of heterobifunctional crosslinking agents include, m-Maleimidobenzoyl-N-hydroxysuccinimide ester (MDS), N-γ-Maleimido butyryloxy succinimide ester (GMBS), N-(ε-Maleimidocaproyloxy) succinimide ester (EMCS) or N-(ε-Maleimidocaproyloxy) sulfo succinimide ester (sulfo-EMCS). In some embodiments, more than one type of crosslinking may be used within a given polypeptide molecule. In some embodiments, crosslinking is accomplished by oxidation, reduction, UV radiation, esterification, hydrolysis, intercalating agents, neoplastic agents, formaldehyde, formalin, silica compounds, siloxane bridges, or photo-crosslinking. In some embodiments, linkers such as small organic molecules (esters, amines) or inorganic molecules (silicas, siloxanes), including microparticles or nanoparticles thereof, may be used to crosslink multiple molecules.

Polypeptides of the disclosure are in some instances made up of multiple polypeptide chains brought together by covalent bonds or by non-covalent forces to form dimers, trimers, tetramers, pentamers, hexamers, septamers, oligomers (<50 polypeptide chains) or, multimers (>50 polypeptide chains). Each of such proteins may have multiple N- and C-termini. Alternatively, the termini of the polypeptides may be modified such that they begin or end, as the case may be, with a non-polypeptide based moiety such as an organic conjugate.

In some embodiments, lipoprotein systems or polypeptides of the disclosure may be modified to reduce their immunogenicity. Immunogenicity is the result of a series of responses to a substance that is perceived as foreign and may include the production of neutralizing and non-neutralizing antibodies, formation of immune complexes, complement activation, mast cell activation, inflammation, hypersensitivity responses, and anaphylaxis. Several factors can contribute to protein immunogenicity, including, but not limited to protein sequence, route and frequency of administration and patient population. Protein engineering may be used to reduce the immunogenicity of the compositions of the disclosure. Modifications to reduce immunogenicity may include those that reduce binding of the processed peptides derived from the sequence of the compositions of the disclosure, to the MHC proteins. For example, amino acids may be modified such that virtually none or a minimal of number of immune epitopes predicted to bind to any prevalent MHC alleles are present in the compositions of the disclosure. Several methods to identify MHC binding epitopes of known protein sequences are known in the art and may be used to score epitopes in the compositions of the present disclosure. Such methods are disclosed in US Patent Publication No. US20020119492, US20040230380, and US 20060148009; the contents of each of which are incorporated by reference in their entirety.

Epitope identification and subsequent sequence modification may be applied to reduce immunogenicity. The identification of immunogenic epitopes may be achieved either physically or computationally. Physical methods of epitope identification may include, for example, mass spectrometry and tissue culture/cellular techniques. Computational approaches that utilize information related to antigen processing, loading and display, structural and/or proteomic data for identifying peptides that may result from antigen processing, and that are likely to have good binding characteristics in the groove of the MHC may also be utilized. One or more mutations may be introduced into the polypeptides of the disclosure to render the identified epitope less or non-immunogenic, while maintaining functionality.

In some embodiments, protein modifications into the structure of the compositions of the disclosure to interfere with antigen processing and peptide loading such as glycosylation and PEGylation, may also be useful in the present disclosure. Compositions of the disclosure may also be to include non-classical amino acid sidechains. Any of the methods discussed in International Patent Publication WO2005051975 for reducing immunogenicity may be useful in the present disclosure (the contents of which are incorporated by reference in their entirety).

In some embodiments, the polypeptides of the disclosure may include a modified amino acid or an amino acid derivative. The term “modified amino acid” or “amino acid derivative” as used herein, refers to a non-proteinogenic amino acid, i.e., an amino acid that is not one of the 20 common amino acids. In some embodiments, the modified amino acid or amino acid derivative is formed by post-translational modification. In some embodiments, the modified amino acid or amino acid derivative is formed by artificial, synthetic processes. In some embodiments, the modified amino acid or amino acid derivative includes a side chain substituent that is substituted by another substituent. In some embodiments, the modified amino acid or amino acid derivative includes a protecting group or other substituents bound to the functional groups such as amino group or carboxyl group. Exemplary modifications include, but are not limited to carboxylation, hydroxylation, acylation, alkylation, amidation, acetylation, lipidation, lipoylation, hypusine formation, prenylation, glipyatyon, phosphopantetheinylation, retinylidene Schiff base formation, diphthamide formation, amide bond formation, butyrylation, gamma-carboxylation, glycosylation, polysialylation, iodination: nucleotide addition, phosphate ester or phosphoramidate formation, phosphorylation, adenylylation, uridylylation, propionylation, pyroglutamate formation, S-glutathionylation, S-nitrosylation, S-sulfenylation, S-sulfinylation, S-sulfonylation, succinylation, glycation, carbamylation, carbonylation, biotinylation, oxidation, pegylation, ISGylation, SUMOylation, ubiquitination, neddylation, pupylation, citrullination, deimination, deamidation, eliminylation, and disulfide bridges.

### Apolipoproteins and Apolipoprotein Variants

In some embodiments, the polypeptides of the disclosure may include or may be derived from a parent polypeptide such as an apolipoprotein. As used herein, an “apolipoprotein” may be used to refer to a protein that binds to and/or is capable of interacting with a lipid. Binding or interaction of the apolipoprotein with the lipid may be direct or indirect.

The apolipoprotein can be in prepro-form (also herein preprotein form), mature, or processed form. For example, when the apolipoprotein is initially expressed in the endoplasmic reticulum as a preproprotein or prepro-form. Preprotein form may include a signal sequence which may be removed following transport across the Golgi apparatus and to its target location e.g., extracellular space. This form may herein be referred to as the “mature form.” Subsequently, a portion of the amino acid sequence may be cleaved by the action of intracellular or extracellular proteolytic cleavage enzymes generating the processed form of the apolipoprotein.

Apolipoproteins of the disclosure may be apolipoprotein E (APOE), apolipoprotein A (APOA), apolipoprotein B (APOB), apolipoprotein C (APOC), apolipoprotein D (APOD), apolipoprotein F (APOF), apolipoprotein H (APOH), apolipoprotein J (APOJ), apolipoprotein L (APOL), apolipoprotein M (APOM), or apolipoprotein O (APOO) or any known apolipoprotein. In a preferred embodiment, the apolipoprotein is an apolipoprotein E.

In some embodiments, the apolipoprotein may be a human apolipoprotein. In some embodiments, the apolipoprotein may be a non-human apolipoprotein. Non-limiting examples of non-human species include monkey, chimpanzee, rat, mouse, horse, sheep, cat, dog, cow, or rabbit.

### Apolipoprotein Variant

In some embodiments, the apolipoprotein may differ in some respect from other forms of the apolipoprotein (i.e., is a variant of another apolipoprotein). Such apolipoproteins or may be a parental apolipoprotein or a starting apolipoprotein polypeptide or a polynucleotide having differences in structure, sequence, or function which distinguish the apolipoprotein variant from starting or reference standard.

Apolipoprotein variants of the disclosure may be a variant of apolipoprotein E (APOE), apolipoprotein A (APOA), apolipoprotein B (APOB), apolipoprotein C (APOC), apolipoprotein D (APOD), apolipoprotein F (APOF), apolipoprotein H (APOH), apolipoprotein J (APOJ), apolipoprotein L (APOL), apolipoprotein M (APOM), or apolipoprotein O (APOO) or any known apolipoprotein.

In general, apolipoprotein variants may possess at least about 70%, at least 80% or at least 90% identity to other forms of the protein. Percentage identity as used herein is defined as the number of residues (amino acid or nucleic acid) per 100 residues of a first sequence that are identical with a second amino acid or nucleic acid sequence after aligning the sequences and introducing gaps, if necessary, to achieve the maximum percentage identity. The final value of percentage identity may be affected by gaps and penalties introduced in the calculation to achieve the maximum value. Percentage identity for an apolipoprotein may be calculated for the entire apolipoprotein or only a for a region or portion thereof. In some embodiments, percentage identity may be at least 70%, 75%, 80%, 90%, 91%, 92%, 93%, 94% 95%, 96%, 97%, 98%, 99%, 99.1%, 99.2%, 99.3%, 99.4%, 99.5%, 99.6%, 99.7%, 99.8%, or 99.9%. In some embodiments, percentage identity may be at least 60%-70%, 65%-75%, 70%-80%, 75%-85%, 85%-95%, 90-99%, or 95-99.99%. Percentage identity of the polypeptides to known apolipoproteins may be from 10% to 20%, from 10% to 25%, from 10% to 50%, from 10% to 60%, from 10% to 70%, from 10% to 80%, from 10% to 90%, from 10% to 95%, from 20% to 25%, from 20% to 50%, from 20% to 60%, from 20% to 70%, from 20% to 80%, from 20% to 90%, from 20% to 95%, from 20% to 99%, from 50% to 60%, from 50% to 70%, from 50% to 80%, from 50% to 90%, from 50% to 95%, from 50% to 99%, from 70% to 80%, from 70% to 90%, from 70% to 95%, from 70% to 99%, from 80% to 90%, from 80% to 95%, from 80% to 99%, from 90% to 95%, from 90% to 99%, and from 95% to 99%, but less than 100%. Percentage identity to a particular reference polynucleotide or polypeptide may be determined by sequence alignment programs e.g., the BLAST suite (Stephen F. Altschul, et al. (1997), “Gapped BLAST and PSI-BLAST: a new generation of protein database search programs,” Nucleic Acids Res. 25:3389-3402). The terms “identical” or percent “identity,” in the context of two or more nucleic acids or polypeptide sequences, refer to two or more sequences or subsequences that are the same or have a specified percentage of amino acid residues or nucleotides that are the same (i.e., 30%, 35%, 40%, 45%, 50%, 55%, 60%, 65%, 70%, 75%, 80%, 85%, 90%, 95%, 98%, 99%, 99.5%, 99.8%, 99.9%, or 100% identity over a specified region, e.g., of the entire polypeptide sequences of the disclosure or individual domains of the polypeptides of the disclosure), when compared and aligned for maximum correspondence over a comparison window, or designated region as measured using a sequence comparison algorithm or by manual alignment and visual inspection. Such sequences that are at least about 80% identical are said to be “substantially identical.” In some embodiments, two sequences are 100% identical. In some embodiments, two sequences are 100% identical over the entire length of one of the sequences (e.g., the shorter of the two sequences where the sequences have different lengths). In various embodiments, identity may refer to the complement of a test sequence.

In some embodiments, the identity exists over a region that is at least about 2 to about 400 amino acids or nucleotides in length. In some embodiments, the identity exists over a region that is at least about 2 to about 390, at least about 2 to about 380, at least about 2 to about 370, at least about 2 to about 360, at least about 2 to about 350, at least about 2 to about 340, at least about 2 to about 330, at least about 2 to about 320, at least about 2 to about 310, at least about 2 to about 300, at least about 2 to about 290, at least about 2 to about 280, at least about 2 to about 270, at least about 2 to about 260, at least about 2 to about 250, at least about 2 to about 200, at least about 2 to about 150, at least about 2 to about 100 amino acids or nucleotides in length. In some embodiments, the identity exists over a region that is at least about 2 to about 90, at least about 2 to about 85, at least about 2 to about 80, at least about 2 to about 75, at least about 2 to about 70, at least about 2 to about 65, at least about 2 to about 60, at least about 2 to about 55, at least about 2 to about 50, at least about 2 to about 45, at least about 2 to about 40, at least about 2 to about 35, at least about 2 to about 30, at least about 2 to about 25, at least about 2 to about 20, at least about 2 to about 10, at least about 2 to about 5 amino acids or nucleotides in length. In some embodiments, the identity exists over a region that is at least about 3 to about 400, about 4 to about 400, about 5 to about 400, about 6 to about 400, about 7 to about 400, about 8 to about 400, about 9 to about 400, about 10 to about 400, about 11 to about 400, about 12 to about 400, about 13 to about 400, about 14 to about 400, about 15 to about 400, about 16 to about 400, about 17 to about 400, about 18 to about 400, about 19 to about 400, about 20 to about 400, about 21 to about 400, about 22 to about 400, about 23 to about 400, about 24 to about 400, about 25 to about 400, about 26 to about 400, about 27 to about 400, about 28 to about 400, about 29 to about 400, about 30 to about 400, about 31 to about 400, about 32 to about 400, about 33 to about 400, about 34 to about 400, about 35 to about 400 amino acids or nucleotides in length. In some embodiments, the identity exists over a region that is at least about 40 to about 400, about 45 to about 400, about 50 to about 400, about 55 to about 400, about 60 to about 400, about 61 to about 400, about 62 to about 400, about 63 to about 400, about 64 to about 400, about 65 to about 400, about 66 to about 400, about 67 to about 400, about 68 to about 400, about 69 to about 400, about 70, to about 400, about 71 to about 400, about 72 to about 400, about 73 to about 400, about 74 to about 400, about 75 to about 400, about 80 to about 400, about 85 to about 400, about 90 to about 400, about 100 to about 400, about 150 to about 400, about 200 to about 400, about 250 to about 400, about 300 to about 400, about 350 to about 400 amino acids or nucleotides in length. In some embodiments, the identity exists over a region that is at least about 2 to about 343, about 3 to about 343, about 4 to about 343, about 7 to about 343, about 9 to about 343, about 11 to about 343, about 15 to about 343, about 16 to about 343, about 20 to about 343, about 25 to about 343, about 62 to about 343, about 2 to about 317, about 3 to about 317, about 4 to about 317, about 7 to about 317, about 9 to about 317, about 11 to about 317, about 15 to about 317, about 16 to about 317, about 20 to about 317, about 25 to about 317, about 62 to about 317, about 2 to about 300, about 3 to about 300, about 4 to about 300, about 7 to about 300, about 9 to about 300, about 11 to about 300, about 15 to about 300, about 16 to about 300, about 20 to about 300, about 25 to about 300, about 62 to about 300, about 2 to about 62, about 3 to about 62, about 4 to about 62, about 7 to about 62, about 9 to about 62, about 11 to about 62, about 15 to about 62, about 16 to about 62, about 20 to about 62, about 25 to about 62 amino acids or nucleotides in length.

In some embodiments, the apolipoprotein variant may be substitutional variant of another apolipoprotein. “Substitutional variants” when referring to polypeptides are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule. In some embodiments, the apolipoproteins may include conservative amino acid substitutions. As used herein, the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. In some embodiments, apolipoprotein variants may include non-conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

In some embodiments, the apolipoprotein variant may be an insertional variant. As used herein, the term “insertional variants” when referring to polypeptides are those with one or more amino acids inserted immediately adjacent to an amino acid at a particular position in a native or starting sequence. As used herein, the term “immediately adjacent” refers to an adjacent amino acid that is connected to either the alpha-carboxy or alpha-amino functional group of a starting or reference amino acid.

In some embodiments, the apolipoprotein variant may be a deletional variant. As used herein, the term “deletional variants” when referring to polypeptides, are those with one or more amino acids in the native or starting amino acid sequence removed. In general, deletional variants will have one or more amino acids deleted in a particular region of the polypeptide.

Systems of the disclosure may also include peptides that mimic the structure and/or activity of an apolipoprotein. As a non-limiting example, the present disclosure may include any of the agonist described in U.S. Pat. Nos. 6,004,925, 6,037,323, 6,046,166, 5,840,688; the contents of each of which are incorporated herein by reference in their entirety.

In some embodiments, the apolipoprotein may be derived from or may be a variant of a non-human apolipoprotein. The non-human species may be monkey, chimpanzee, rat, mouse, horse, sheep, cat, dog, cow, and/or rabbit.

Apolipoproteins or apolipoprotein variants of the disclosure may have an N-terminal or C-terminal truncation or may have one or more internal deletions or insertions with respect to other forms of the apolipoprotein polypeptide. An apolipoprotein used in the methods and compositions of the disclosure may be a multimer of an apolipoprotein or a portion thereof, for example, two or more copies of an apolipoprotein, or a variant or portion thereof, joined by a linker. An apolipoprotein used in the methods and compositions of the disclosure may be a chimeric apolipoprotein, comprising sequences of two different apolipoproteins (or variants thereof). Furthermore, the apolipoprotein may be bound to a peptide or another protein sequence, e.g., as part of a fusion protein. The peptide sequence may be a purification and/or detection tag, for example.

Non-limiting examples of apolipoproteins are provided in Table 1.

TABLE 1
Apolipoproteins

| Gene name | ENSEMBL | Protein |
|---|---|---|
| (variant name) | PRT ID | SEQ ID NO: |
| APOE (APOE3) | ENSP00000252486 | 1 |
| APOE (APOE2) | — | 2 |
| APOE (APOE4) | — | 3 |
| APOE Variant | ENSP00000413135 | 4 |
| APOE Variant | ENSP00000413653 | 5 |
| APOE Variant | ENSP00000410423 | 6 |
| APOE Variant | — | 7 |
| APOE Variant | — | 8 |
| APOE Variant | — | 9 |
| APOE Variant | — | 10 |

In some embodiments, the apolipoprotein is a variant or mutation of a protein in Table 1.

### APOE

Polypeptides of the disclosure may be Apolipoprotein E (APOE) or a derivative and/or variant of from APOE. This family of proteins binds lipids and interacts with low density lipoprotein receptor (LDLR) which is important in processing of triglyceride rich proteins. In peripheral tissues, APOE may be produced by the liver and macrophages, and mediates cholesterol metabolism. In the central nervous system, APOE is produced by astrocytes and transports cholesterol to neurons via APO receptors, which are members of the LDLR family.

APOE is widely distributed across lipoprotein classes: it is present in chylomicron remnants, mature very low-density lipoprotein (VLDL), VLDL remnants, low density lipoprotein (LDL) and high density lipoprotein (HDL). It binds with a high affinity to the LDL-receptor as well as to the LRP family of receptors. APOE facilitates clearance of chylomicron and VLDL remnants. Approximately 60% of plasma APOE is present in the HDL, which exchanges it with other lipoproteins. APOE is synthesized by hepatocytes, macrophages and by brain astrocytes. APOE controls cholesterol efflux from cells together with APOA. It also has antioxidant properties and plays a role in the regulation of inflammatory response. Different variants of APOE may have different affinity for lipids, lipoproteins and receptors and selection of an APOE variant for inclusion in the lipoprotein systems of the disclosure may be adjusted accordingly.

Apolipoproteins of the present disclosure may be modifications (e.g., mutations) of other apolipoproteins (e.g., APOE, APOE1, APOE2, APOE3, APOE4, or APOE5). Apolipoprotein mutations include may include amino acid substitution, deletion, insertion, and amino acid sequence alteration (e.g., reverse oriented sequence, scrambled sequence, multidomain sequence, switched-domain sequence).

In some embodiments, the APOE variant may be APOE3 (ENSP00000252486; SEQ ID NO: 1), APOE2 (C130, C176) (SEQ ID NO: 2), APOE4 (R130, R176) (SEQ ID NO: 3). This precursor form of the protein includes the signal sequence.

In some embodiments, the apolipoprotein may be a region of another apolipoprotein, for example, amino acids (aa) 18-317 of APOE3 (SEQ ID NO: 268; aa 18-317 of APOE2 (SEQ ID NO: 267); aa 18-317 of APOE4 (SEQ ID NO: 269).

In some embodiments, polypeptides of the disclosure may be APOE variants associated with a disease or disorder. In some embodiments, the polypeptides of the disclosure may be APOE variants associated with a disease such as, but not limited to a neurological disorder (e.g., Alzheimer's disease), a cardiovascular disease (e.g., hyperlipoproteinemia, familial dysbetalipoproteinemia, hypercholesterolemia, familial combined hyperlipidemia), or a renal disease (e.g., lipoprotein glomerulopathy). APOE variants have been identified as major genetic modifier of Alzheimer's disease (AD) contributing to the susceptibility of late-onset AD. APOE impacts amyloid production, aggregation, and clearance, is a component of amyloid plaques, and exacerbates tau-mediated neurodegeneration. APOE2 confers some protection against Alzheimer's disease, whereas APOE4 has been associated with increased risk of Alzheimer's disease (Corder et al., Nat Genet. 1994; 7:180-4; Corder et al., Science. 1993; 261:921-3; Poirier et al., Lancet. 1993; 342:697-9; the contents of each of which are herein incorporated by reference in its entirety). APOE also regulates lipid metabolism and cardiovascular risk. About 5-10% of APOE2 homozygote individuals develop hyperlipoproteinemia type III (HLP III), whereas other APOE variants are linked to autosomal dominant HLP III. HLP III is characterized by increased plasma cholesterol and triglycerides levels and by the presence of tuberous or striated palmar xanthomas. In some embodiments, the polypeptides of the disclosure may be an APOE variant associated with cognitive decline in aging. Individuals with APOE-ε4 allele show decline in memory before the age of 60 years and exhibited greater acceleration than non-carriers (Caselli et al., N Engl J Med. 2009; 361:255-63).

In some embodiments, the APOE variant may be an APOE Christchurch variant (also referred to herein as APOECh or APOE3ChC). As used herein, the term APOE Christchurch variant may be used to refer an APOE protein sequence that includes a serine at position 154 of APOE protein sequence (SEQ ID NO: 1-3). APOE Christchurch as used herein may also be referred to as APOE (R136S) or APOE of SEQ ID NO: 268, 267, 269, which include amino acid (aa) 18-317 of SEQ ID NO: 1-3. In some embodiments, the APOE Christchurch variant may be an APOE3 Christchurch variant (also herein referred to as APOE3Ch, APOE3-Ch, APOE3ChC or APOE3 (R154S) (SEQ ID NO: 330). In some embodiments, the APOE Christchurch variant may be an APOE2 Christchurch variant (also herein referred to as APOE2Ch, APOE2-Ch, APOE2ChC or APOE2 (R154S). The ability of the APOE Christchurch variant to suppress tau pathology was corroborated with in vitro experiments demonstrating that APOE Christchurch variant may behave like the APOE2; both bind poorly to heparin (APOE3ChC<APOE2) (Mahley et al., 1999 Lipid Res. 40, 1933-1949 (1999); Arboleda-Velsquez et al. 2019, Nat Med 25, 1680-1683; the contents of each of which are herein incorporated by reference in its entirety). These findings suggest that the AD-related chain of pathogenic events is likely broken by APOE3ChC after amyloid formation, and independent of the mechanism, the Christchurch mutation is protective.

In some embodiments, the APOE variant may be an APOE Jacksonville variant (also referred to herein as APOEJac). As used herein, the term APOE Jacksonville variant may be used to refer to an APOE protein sequence that includes a glutamic acid at position 254 (V254E) of APOE protein sequence (SEQ ID NO: 1-3). APOE Jacksonville as used herein may also be referred to as APOE (V236E) relative to APOE of SEQ ID NO: 268, 267, or 269, which include amino acid (aa) 18-317 of SEQ ID NO: 1-3. In some embodiments, the APOE Jacksonville variant may be an APOE3Jacksonville variant (also herein referred to as APOE3Jac or APOE3(V254E)) (SEQ ID NO: 331). In some embodiments, the APOE Jacksonville variant may be an APOE2 Jacksonville variant (also herein referred to as APOE2Jac or APOE3 (V254E). Liu et al. have found that APOE Jacksonville variant associated with healthy brain aging and reduced risk for AD and dementia with Lewy bodies (DLB) (Liu C C et al. Sci Transl Med. 2021 Sep. 29; 13(613):eabc9375; the contents of which are herein incorporated by reference in its entirety).

The APOE variants of the disclosure may include one or more mutations at the positions discussed herein with respect to APOE3 (SEQ ID NO:1), APOE2 (SEQ ID NO:2), APOE4 (SEQ ID NO:3).

In some embodiments, the APOE protein may be a mutant of human APOE3 (SEQ ID NO: 1; Reference Ensemble PRT ID ENSP00000252486). Mutants relative to APOE3 include A6T (SEQ ID NO: 388), A7V (SEQ ID NO: 389), L9P (SEQ ID NO: 390), V10I (SEQ ID NO: 391), T11A (SEQ ID NO: 392), T11S (SEQ ID NO: 393), F12Y (SEQ ID NO: 394), F12L (SEQ ID NO: 395), L13R (SEQ ID NO: 396), A18P (SEQ ID NO: 397), A18T (SEQ ID NO: 398), E21K (SEQ ID NO: 399), A23V (SEQ ID NO: 400), V24L (SEQ ID NO: 401), T26_E29dup (SEQ ID NO: 402), P28L (SEQ ID NO: 403), E31K (SEQ ID NO: 404), R33C (SEQ ID NO: 405), R33H (SEQ ID NO: 406), Q35R (SEQ ID NO: 407), E37K (SEQ ID NO: 408), Q39R (SEQ ID NO: 409), G41S (SEQ ID NO: 410), R43del (SEQ ID NO: 411), R43C (SEQ ID NO: 412), R43H (SEQ ID NO: 413), L46P (SEQ ID NO: 415), L48V (SEQ ID NO: 416), R50C (SEQ ID NO: 417), R50S (SEQ ID NO: 418), R50G (SEQ ID NO: 419), R50P (SEQ ID NO: 420), R50H (SEQ ID NO: 421), R50L (SEQ ID NO: 422), R56H (SEQ ID NO: 423), T60A (SEQ ID NO: 424), Q64H (SEQ ID NO: 425), E68del (SEQ ID NO: 426), E68V (SEQ ID NO: 428), E68D (SEQ ID NO: 429), L69Q (SEQ ID NO: 430), L69P (SEQ ID NO: 431), L70F (SEQ ID NO: 432), Q73R (SEQ ID NO: 433), V74A (SEQ ID NO: 434), Q76R (SEQ ID NO: 435), E77K (SEQ ID NO: 436), A80E (SEQ ID NO: 437), M82R (SEQ ID NO: 438), D83E (SEQ ID NO: 439), E84K (SEQ ID NO: 440), M86V (SEQ ID NO: 441), E88Q (SEQ ID NO: 442), L89S (SEQ ID NO: 443), K90E (SEQ ID NO: 444), K90N (SEQ ID NO: 445), Y92C (SEQ ID NO: 446), K93fs (SEQ ID NO: 447), K93T (SEQ ID NO: 448), K93R (SEQ ID NO: 449), K93N (SEQ ID NO: 450), S94* (SEQ ID NO: 451), Q99K (SEQ ID NO: 452), Q99R (SEQ ID NO: 453), P102R (SEQ ID NO: 454), V103L (SEQ ID NO: 455), V103L (SEQ ID NO: 456), A104T (SEQ ID NO: 457), E106Q (SEQ ID NO: 458), R108_A118dup (SEQ ID NO: 459), R108Q (SEQ ID NO: 460), R110fs (SEQ ID NO: 461), R110Q (SEQ ID NO: 461), S112Y (SEQ ID NO: 462), K113E (SEQ ID NO: 463), E114K (SEQ ID NO: 464), L115R (SEQ ID NO: 465), A117V (SEQ ID NO: 466), Q119P (SEQ ID NO: 467), A120S (SEQ ID NO: 468), A120D (SEQ ID NO: 469), R121W (SEQ ID NO: 470), G123R (SEQ ID NO: 471), D128N (SEQ ID NO: 472), V129M (SEQ ID NO: 473), G131C (SEQ ID NO: 474), R132C (SEQ ID NO: 475), R132L (SEQ ID NO: 476), Q135R (SEQ ID NO: 477), Y136C (SEQ ID NO: 478), R137C (SEQ ID NO: 479), R137G (SEQ ID NO: 480), R137H (SEQ ID NO: 481), G138C (SEQ ID NO: 482), V140L (SEQ ID NO: 483), V140M (SEQ ID NO: 484), L1441 (SEQ ID NO: 485), G145R (SEQ ID NO: 486), G145S (SEQ ID NO: 487), G145D (SEQ ID NO: 488), G145A (SEQ ID NO: 489), Q146* (SEQ ID NO: 490), L151M (SEQ ID NO: 491), R152W (SEQ ID NO: 492), R152Q (SEQ ID NO: 493), V153M (SEQ ID NO: 494), R154S (SEQ ID NO: 330), R154fs (SEQ ID NO: 495), R154C (SEQ ID NO: 496), R154L (SEQ ID NO: 497), S157F (SEQ ID NO: 498), L159P (SEQ ID NO: 499), R160G (SEQ ID NO: 500), 163C (SEQ ID NO: 501), R163H (SEQ ID NO: 502), K164Q (SEQ ID NO: 503), L167del (SEQ ID NO: 504), R168H (SEQ ID NO: 505), D169Y (SEQ ID NO: 506), D172N (SEQ ID NO: 507), Q174K (SEQ ID NO: 508), V179A (SEQ ID NO: 509), Y180H (SEQ ID NO: 510), Q181E (SEQ ID NO: 511), Q181H (SEQ ID NO: 512), A184S (SEQ ID NO: 513), G187A (SEQ ID NO: 514), G187V (SEQ ID NO: 515), G191_R198dup (SEQ ID NO: 516), E189K (SEQ ID NO: 517), R190C (SEQ ID NO: 518), G191S (SEQ ID NO: 519), S193R (SEQ ID NO: 520), R196C (SEQ ID NO: 521), R198C (SEQ ID NO: 522), G200R (SEQ ID NO: 523), R207S (SEQ ID NO: 524), R207C (SEQ ID NO: 525), R207H (SEQ ID NO: 526), R207P (SEQ ID NO: 527), V208L (SEQ ID NO: 528), V208M (SEQ ID NO: 529), V213G (SEQ ID NO: 530), G214S (SEQ ID NO: 531), G218R (SEQ ID NO: 532), G218S (SEQ ID NO: 533), Q219E (SEQ ID NO: 534), P220L (SEQ ID NO: 535), L221 P (SEQ ID NO: 536), Q222* (SEQ ID NO: 537), Q222K (SEQ ID NO: 538), Q222R (SEQ ID NO: 539), R224P (SEQ ID NO: 540), R224L (SEQ ID NO: 541), A225T (SEQ ID NO: 542), A227S (SEQ ID NO: 543), W228* (SEQ ID NO: 544), G229R (SEQ ID NO: 545), G229V (SEQ ID NO: 546), E230K (SEQ ID NO: 547), R231Q (SEQ ID NO: 548), A234T (SEQ ID NO: 549), A234V (SEQ ID NO: 550), R235W (SEQ ID NO: 551), R235Q (SEQ ID NO: 552), E238del (SEQ ID NO: 553), E238D (SEQ ID NO: 554), M2391 (SEQ ID NO: 555), G240R (SEQ ID NO: 556), S241R (SEQ ID NO: 557), T243fs (SEQ ID NO: 558), R242W (SEQ ID NO: 559), R242G (SEQ ID NO: 560), R242P (SEQ ID NO: 561), T243N (SEQ ID NO: 562), R246S (SEQ ID NO: 563), R246C (SEQ ID NO: 564), R246P (SEQ ID NO: 565), D248E (SEQ ID NO: 566), E249K (SEQ ID NO: 567), E249Q (SEQ ID NO: 568), E256V (SEQ ID NO: 569), R258H (SEQ ID NO: 570), A259P (SEQ ID NO: 571), E262K (SEQ ID NO: 572), E263K (SEQ ID NO: 573), Q267R (SEQ ID NO: 574), V254E (SEQ ID NO: 331), R269fs (SEQ ID NO: 575), R269C (SEQ ID NO: 576), R269G (SEQ ID NO: 577), E273G (SEQ ID NO: 578), A274G (SEQ ID NO: 579), Q276* (SEQ ID NO: 580), A277V (SEQ ID NO: 581), R278C (SEQ ID NO: 582), L279fs (SEQ ID NO: 583), W282C (SEQ ID NO: 584), E284G (SEQ ID NO: 585), E284D (SEQ ID NO: 586), L286M (SEQ ID NO: 587), V287M (SEQ ID NO: 588), D289A (SEQ ID NO: 589), R292C (SEQ ID NO: 590), R292H (SEQ ID NO: 591), W294C (SEQ ID NO: 592), A295T (SEQ ID NO: 593), G296R (SEQ ID NO: 594), G296W (SEQ ID NO: 595), L297P (SEQ ID NO: 596), E299Q (SEQ ID NO: 597), K300R (SEQ ID NO: 598), K300N (SEQ ID NO: 599), V301L (SEQ ID NO: 600), A303S (SEQ ID NO: 601), V305M (SEQ ID NO: 602), T3071 (SEQ ID NO: 603), A309T (SEQ ID NO: 604), P311A (SEQ ID NO: 605), S314R (SEQ ID NO: 606), *318ext (SEQ ID NO: 607), W38* (SEQ ID NO: 608), E98fs (SEQ ID NO: 609), A117T (SEQ ID NO: 610), L122M (SEQ ID NO: 611), Q141R (SEQ ID NO: 612), R160C (SEQ ID NO: 613), R163P (SEQ ID NO: 614), K164E (SEQ ID NO: 615), R165P (SEQ ID NO: 616), A170P (SEQ ID NO: 617), and G183A (SEQ ID NO: 618). In some embodiments, the APOE protein may include more than one mutation relative to APOE3 (SEQ ID NOs.: 1-3), e.g., more than two mutations, more than three mutations, more than four mutations, more than five mutations, etc.

In some embodiments, the APOE protein may be a mutant of SEQ ID NO: 5. Exemplary mutations include S21 (SEQ ID NOs: 619), G4E (SEQ ID NOs: 620), A5T (SEQ ID NOs: 621), S6F (SEQ ID NOs: 622), R7K (SEQ ID NOs: 623), P12S (SEQ ID NOs: 624), N14K (SEQ ID NOs: 625), P17S (SEQ ID NOs: 626), P18L (SEQ ID NOs: 627), D19N (SEQ ID NOs: 628), W20C (SEQ ID NOs: 629), 122fs (SEQ ID NOs: 630), T23K (SEQ ID NOs: 631), G24D (SEQ ID NOs: 632), G24V (SEQ ID NOs: 633), and K26fs (SEQ ID NOs: 634) (see SEQ ID NOs: 619-634).

### APOA

In some embodiments, the polypeptides of the disclosure may include or may be derived from APOA. In some embodiments, APOA may be APOA1 (also herein referred to as apolipoprotein A-I, Apo A-I, ApoA-I, Apo A1, ApoA1), APOA2 (also herein referred to as apolipoprotein A-II, Apo A-II, ApoA-II, Apo A2, ApoA2) APOA4 (also herein referred to as apolipoprotein A-IV, Apo A-IV, ApoA-IV, Apo A4, ApoA4), and/or APOA5 (also herein referred to as apolipoprotein A-V, Apo A-V, ApoA-V, Apo A5, ApoA5).

In some embodiments, the polypeptides of the disclosure include or are derived from APOA1 or a variant thereof. APOA1 is a protein of 243 amino acids. The APOA gene is located on chromosome 11 and is part of the APOA1/C3/A4/A5 gene cluster. It is synthesized in the liver and intestine. The concentration of APOA1 is controlled by its degradation rate. APOA1 constitutes 70% of HDL apolipoproteins. It activates LCAT and is also an anti-inflammatory molecule and an antioxidant. Plasma levels of APOA1 vary and depend on method of measurement and population.

In some embodiments, the polypeptides of the disclosure include or are derived from APOA2 or a variant thereof. APOA2 is a 77-amino acid homodimer with molecular mass of 17,400 Da. It is predominantly expressed in the liver. APOA2 accounts for approximately 20% of HDL protein. HDL particles contain just APOA1 or both APOA1 and APOA2. APOA2 concentration is determined by its production rate. It inhibits LPL activity (and may also inhibit HTGL) and serves as a co-factor for LCAT and CETP.

In some embodiments, the polypeptides of the disclosure include or are derived from APOA4 or a variant thereof. APOA4 is a glycoprotein with a molecular weight of 46,000 Da. It is synthesized in the intestine and is incorporated into nascent chylomicrons. It is also present in HDL. Because it is relatively hydrophilic, it may be displaced from lipoproteins by APOE and circulates predominantly as lipid-free protein. It participates in intestinal lipid absorption, in the assembly of chylomicrons and affects various aspects of lipoprotein response to diet.

In some embodiments, the polypeptides of the disclosure include or are derived from APOA5 or a variant thereof. APOA5 modulates hepatic VLDL synthesis and secretion. The gene coding for APOA5 has been strongly associated with triglyceride (TG) concentration. APOA5 deficiency leads to type V dyslipidemia and to reduced post-heparin LPL activity. On the other hand, its overexpression leads to a decreased TG concentration. APOA5 is synthesized in the liver.

In some embodiments, the APOA variant may include mutations R175C (herein referred to as APOA Paris or APOA1 Paris) and/or R197C (herein referred to as APOA Milano or APOA1 Milano) with respect to ENSP00000236850 (SEQ ID NO: 9).

The APOA variants of the disclosure may include one or more mutations at the positions discussed herein with respect to REF ENSMBL PRT ID ENSP00000236850. Mutations relative to AOPA1 include: Q267P (SEQ ID NO: 635), Q267E (SEQ ID NO: 636), Q267* (SEQ ID NO: 637), N265dup (SEQ ID NO: 638), T261S (SEQ ID NO: 639), A256T (SEQ ID NO: 640), L254R (SEQ ID NOs: 641), Q240fs (SEQ ID NOs: 642), F253L (SEQ ID NOs: 643), V251 D (SEQ ID NOs: 644), L243V (SEQ ID NOs: 645), L242M (SEQ ID NO: 646), L238del (SEQ ID NO: 647), L238P (SEQ ID NO: 648), E236K (SEQ ID NO: 649), P233L (SEQ ID NO: 650), K230del (SEQ ID NO: 651), E229D (SEQ ID NO: 652), E229K (SEQ ID NO: 653), S228R (SEQ ID NO: 654), S225G (SEQ ID NO: 655), E222K (SEQ ID NO: 656), E222* (SEQ ID NO: 657), T221S (SEQ ID NO: 658), A220P (SEQ ID NO: 659), A218G (SEQ ID NO: 660), E215Q (SEQ ID NO: 661), L213V (SEQ ID NO: 662), R212S (SEQ ID NO: 663), R2121 (SEQ ID NO: 664), G209R (SEQ ID NO: 665), N208Y (SEQ ID NO: 666), E207D (SEQ ID NO: 667), K206R (SEQ ID NO: 668), L205V (SEQ ID NO: 669), A204V (SEQ ID NO: 670), R201H (SEQ ID NO: 671), R201S (SEQ ID NO: 672), A199T (SEQ ID NO: 673), R197S (SEQ ID NO: 674), Q196P (SEQ ID NO: 675), E193K (SEQ ID NO: 676), D192E (SEQ ID NO: 677), D192N (SEQ ID NO: 678), Y190C (SEQ ID NO: 679), P189R (SEQ ID NO: 680), A188G (SEQ ID NO: 681), A188V (SEQ ID NO: 682), A188S (SEQ ID NO: 683), H186fs (SEQ ID NO: 684), R184L (SEQ ID NO: 685), A182V (SEQ ID NO: 686), A182E (SEQ ID NO: 687), A182T (SEQ ID NO: 688), R177L (SEQ ID NO: 689), A176V (SEQ ID NO: 690), A176S (SEQ ID NO: 691), A176T (SEQ ID NO: 692), E171V (SEQ ID NO: 693), E171K (SEQ ID NO: 694), E170K (SEQ ID NO: 695), E170* (SEQ ID NO: 696), L168P (SEQ ID NO: 697), L168V (SEQ ID NO: 698), S166R (SEQ ID NO: 699), L165R (SEQ ID NO: 700), K164N (SEQ ID NO: 701), E163G (SEQ ID NO: 702), E163K (SEQ ID NO: 703), Q162P (SEQ ID NO: 704), E160D (SEQ ID NO: 705), E160K (SEQ ID NO: 706), E160Q (SEQ ID NO: 707), H159Q (SEQ ID NO: 708), Q156R (SEQ ID NO: 709), R155L (SEQ ID NO: 710), A154V (SEQ ID NO: 711), A154T (SEQ ID NO: 712), E152K (SEQ ID NO: 713), Q151* (SEQ ID NO: 714), L150I (SEQ ID NO: 715), R147H (SEQ ID NO: 716), E144K (SEQ ID NO: 717), K142R (SEQ ID NO: 718), R140C (SEQ ID NO: 719), R140S (SEQ ID NO: 720), Y139* (SEQ ID NO: 721), Y139H (SEQ ID NO: 722), M136T (SEQ ID NO: 723), E134A (SEQ ID NO: 724), Q133E (SEQ ID NO: 725), W132C (SEQ ID NO: 726), W132L (SEQ ID NO: 727), K131del (SEQ ID NO: 728), K131M (SEQ ID NO: 729), K130R (SEQ ID NO: 730), Q129P (SEQ ID NO: 731), D127N (SEQ ID NO: 732), D126G (SEQ ID NO: 733), Y124* (SEQ ID NO: 734), P123S (SEQ ID NO: 735), P123T (SEQ ID NO: 736), V121A (SEQ ID NO: 737), A119V (SEQ ID NO: 738), D113E (SEQ ID NO: 739), M1101 (SEQ ID NO: 740), R107S (SEQ ID NO: 741), E104D (SEQ ID NO: 742), E104K (SEQ ID NO: 743), K101 E (SEQ ID NO: 744), E100G (SEQ ID NO: 745), L99P (SEQ ID NO: 746), D97N (SEQ ID NO: 747), F95Y (SEQ ID NO: 748), T921 (SEQ ID NO: 749), L88F (SEQ ID NO: 750), Q87H (SEQ ID NO: 751), K83N (SEQ ID NO: 752), F81Y (SEQ ID NO: 753), F81I (SEQ ID NO: 754), T781 (SEQ ID NO: 755), L71del (SEQ ID NO: 756), L71V (SEQ ID NO: 757), N67K (SEQ ID NO: 758), L66L (SEQ ID NO: 759), G63fs (SEQ ID NO: 760), L62F (SEQ ID NO: 761), A61T (SEQ ID NO: 762), S60P (SEQ ID NO: 763), S60A (SEQ ID NO: 763), G59V (SEQ ID NO: 764), G59A (SEQ ID NO: 765), Y53* (SEQ ID NO: 766), Y53C (SEQ ID NO: 767), G50V (SEQ ID NO: 768), G50S (SEQ ID NO: 769), S49N (SEQ ID NO: 770), S491 (SEQ ID NO: 771), S49R (SEQ ID NO: 772), D44E (SEQ ID NO: 773), V43L (SEQ ID NO: 774), V43M (SEQ ID NO: 775), V41 L (SEQ ID NO: 776), T40A (SEQ ID NO: 777), A39V (SEQ ID NO: 778), R34L (SEQ ID NO: 779), R34Q (SEQ ID NO: 780), P31L (SEQ ID NO: 781), P31A (SEQ ID NO: 782), S30G (SEQ ID NO: 783), S30R (SEQ ID NO: 784), Q29H (SEQ ID NO: 785), Q29K (SEQ ID NO: 786), P28R (SEQ ID NO: 787), P28S (SEQ ID NO: 788), P27T (SEQ ID NO: 789), P27S (SEQ ID NO: 790), R19P (SEQ ID NO: 791), R19W (SEQ ID NO: 792), T14M (SEQ ID NO: 793), or V10M (SEQ ID NO: 794).

The APOA variants of the disclosure may include one or more mutations at the positions discussed herein with respect to REF ENSMBL PRT ID ENSP00000364478. Mutations with respect to REF ENSMBL PRT ID ENSP00000364478 include T226M (SEQ ID NO: 795), G209S (SEQ ID NO: 796), L202P (SEQ ID NO: 797), A199P (SEQ ID NO: 798), L198S (SEQ ID NO: 799), R197C (SEQ ID NO: 800), D181G (SEQ ID NO: 801), V180E (SEQ ID NO: 802), H179fs (SEQ ID NO: 803), R177P (SEQ ID NO: 804), R173P (SEQ ID NO: 805), P167R (SEQ ID NO: 806), E152G (SEQ ID NO: 807), K131* (SEQ ID NO: 808), L114P (SEQ ID NO: 809), Q108* (SEQ ID NO: 810), F95L (SEQ ID NO: 811), L84R (SEQ ID NO: 812), W74R (SEQ ID NO: 813), G50R (SEQ ID NO: 815), P27H (SEQ ID NO: 817), P27R (SEQ ID NO: 818), A218P (SEQ ID NO: 820), R212G (SEQ ID NO: 821), G45R (SEQ ID NO: 822), G45S (SEQ ID NO: 823), R40C (SEQ ID NO: 824), G35S (SEQ ID NO: 825), C34R (SEQ ID NO: 826), A28S (SEQ ID NO: 827), T27M (SEQ ID NO: 828), T27fs (SEQ ID NO: 829), A17_S23dup (SEQ ID NO: 830), F21del (SEQ ID NO: 831), S22P (SEQ ID NO: 832), F21L (SEQ ID NO: 833), F21S (SEQ ID NO: 834), F20L (SEQ ID NO: 835), H15L (SEQ ID NO: 837), P14S (SEQ ID NO: 838), G7S (SEQ ID NO: 839), R6Q (SEQ ID NO: 840), R6W (SEQ ID NO: 841), G4R (SEQ ID NO: 842), G3A (SEQ ID NO: 843), G3R (SEQ ID NO: 844), or M1I (SEQ ID NO: 845).

### Polypeptide Features

In some embodiments, polypeptides of the disclosure may include one or more components or features. As used herein, the term “features” when referring to proteins or polypeptides are defined as distinct amino acid sequence-based components of a molecule. In some embodiments, the polypeptides of the disclosure may include at least one structured sequence region. In some embodiments, the polypeptides of the present disclosure may include from about 2 and about 30 structure sequence regions, e.g., from about 5 to about 25 structured sequence regions, from about 10 to about 20 structured sequence regions, about 15 structured sequence regions, etc. In some embodiments, Polypeptides of the disclosure may further include a signal sequence, an N-terminal sequence region, a C-terminal sequence region, or a hinge region, a linker region, or combinations thereof.

Features of the polypeptides of the present disclosure include surface manifestations, local conformational shape, folds, loops, half-loops, domains, half-domains, sites, termini or any combination thereof.

In some embodiments, the polypeptides of the present disclosure include one or more surface manifestations. As used herein, the term “surface manifestation” when referring to proteins refers to a polypeptide-based component of a protein appearing on an outermost surface.

In some embodiments, the polypeptides of the present disclosure include one or more local confirmational shapes. As used herein, the term “local conformational shape” when referring to proteins refers to a polypeptide based structural manifestation of a protein which is located within a definable space of the protein.

In some embodiments, the polypeptides of the present disclosure include one or more folds. As used herein, the term “fold,” when referring to proteins, refers to the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

In some embodiments, the polypeptides of the present disclosure include one or more turns. As used herein, the term “turn” as it relates to protein conformation, refers to a bend which alters the direction of the backbone of a peptide or polypeptide and may involve one, two, three or more amino acid residues.

In some embodiments, the polypeptides of the present disclosure include one or more loops. As used herein, the term “loop,” when referring to proteins, refers to a structural feature of a peptide or polypeptide which reverses the direction of the backbone of a peptide or polypeptide and includes four or more amino acid residues. Oliva et al. have identified at least 5 classes of protein loops (Oliva, B. et al., An automated classification of the structure of protein loops. J Mol Biol. 1997. 266(4):814-30; the contents of which are herein incorporated by reference in its entirety).

In some embodiments, the polypeptides of the present disclosure include one or more half-loops. As used herein, the term “half-loop,” when referring to proteins, refers to a portion of an identified loop having at least half the number of amino acid residues as the loop from which it is derived. It is understood that loops may not always contain an even number of amino acid residues. Therefore, in those cases where a loop contains or is identified to include an odd number of amino acids, a half-loop of the odd-numbered loop will include the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

In some embodiments, the polypeptides of the present disclosure include one or more motifs and/or domains. A motif is a short sequence (<40 residues) pattern associated with one or more identifiable structural or functional characteristics or properties (e.g., binding capacity, serving as a site for protein-protein interactions). In some embodiments, a motif refers to a distinct structural site performing a particular function. A domain is also a conserved sequence pattern, defined as an independent functional and structural unit. Domains are normally longer than motifs. A domain consists of more than 40 residues and up to 700 residues, with an average length of 100 residues. A domain may or may not include motifs within its boundaries.

In some embodiments, the polypeptides of the present disclosure include one or more half-domains. As used herein, the term “half-domain,” when referring to proteins, refers to a portion of an identified domain having at least half the number of amino acid residues as the domain from which it is derived. It is understood that domains may not always contain an even number of amino acid residues. Therefore, in those cases where a domain contains or is identified to include an odd number of amino acids, a half-domain of the odd-numbered domain will include the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that include any of the domain types herein need not be contiguous along the backbone of the polypeptide (i.e., nonadjacent amino acids may fold structurally to produce a domain, half-domain or subdomain).

In some embodiments, the polypeptides of the present disclosure include one or more sites. As used herein, the terms “site,” as it pertains to amino acid based embodiments is used synonymously with “amino acid residue” and “amino acid side chain”. A site represents a position within a peptide or polypeptide that may be modified, manipulated, altered, derivatized or varied within the polypeptide based molecules of the present disclosure.

Polypeptide features may be manipulated and/or modified by moving, swapping, inverting, deleting, randomizing or duplicating. Furthermore, it is understood that manipulation of features may result in the same outcome as a modification to the compositions of the disclosure. For example, a manipulation which involved deleting a domain would result in the alteration of the length of a molecule just as modification of a nucleic acid to encode less than a full-length molecule would. Modifications and manipulations may be accomplished by methods known in the art such as site directed mutagenesis. The resulting modified molecules may then be tested for activity using in vitro or in vivo assays such as those described herein or any other suitable screening assay known in the art.

In some embodiments, the polypeptides of the disclosure may be modified to tune polypeptide binding to lipid and/or to receptors. Modifications may be introduced into the polypeptides to alter or tune intra or inter polypeptide interactions (for example, Frieden et al, PNAS) (109):8913-8918, 2012, Fan D, et al. Biochemistry. 2004 May 4; 43(17):5055-64, Zhang Y, et al. Biochemistry. 2007 Sep. 18; 46(37):10722-32 and Georgiadou et al, PLoS One (6)e27037, 2011; the contents of each of which are herein incorporated by reference in their entirety). In some embodiments, polypeptides of the disclosure may be modified to alter one or more properties including, but not limited to aggregation, complement binding, half-life and/or stability.

In some embodiments, the polypeptides of the disclosure may include one or more receptor binding domains. As used herein, a receptor binding domain may refer to domain in a parent protein or polypeptide e.g., an apolipoprotein, that interacts with a receptor. Interaction of the domain with the receptor may lead to the cell containing the receptor to perform a particular function and/or acquire a particular phenotype. In some embodiment, the polypeptides of the disclosure may include a receptor binding domain of or derived from APOE. In some embodiments, the receptor may be low density lipoprotein receptor (LDLR), LDL receptor related protein (LRP e.g., LRP1, LRP4, LRP8), very low density lipoprotein receptor (VLDLR), Scavenger receptor class B member (SCARB1), apolipoprotein B receptor (APOBR), nuclear receptor subfamily 1 group H member 2 (NR1H2), and/or nuclear receptor subfamily 2 group F member 2 (NR2F2). In some embodiments the receptor binding domain may include a region beginning at amino acid 120, 125, 130, 135, 140, 141, 142, 143, 144, 145, 146, 147, 148, 149, 150, 151, 151, 152, 153, 154, 155, 156, 157, 158, 159, 160, 165, 170, 175, 180, to amino acid 125, 130, 135, 140, 141, 142, 143, 144, 145, 146, 147, 148, 149, 150,151, 151, 152, 153, 154, 155, 156, 157, 158, 159, 160, 165, 170, 175, 180, 185 of APOE3 (SEQ ID NO: 1), APOE2 (SEQ ID NO: 2), or APOE4 (SEQ ID NO: 3) or a variant thereof. Non-limiting examples of the receptor binding domain may include a region such as aa 130-150, 142-160, 148-168, 154-168, 160-178 of APOE3 (SEQ ID NO: 1), APOE2 (SEQ ID NO: 2), or APOE4 (SEQ ID NO: 3) or a variant thereof. In some embodiments, the receptor biding domain may include a RLASHLRKLRKRLLRDADDLQKRLA (SEQ ID NO: 851). In some embodiments, the receptor binding domain may include one or more mutations to modulate receptor binding properties. The polypeptides of the disclosure may also include one or more mutations to modulate accessibility to the receptor binding sequence. In some embodiments, the compositions of the disclosure may include point mutations in the binding region that increase receptor and matrix binding. In some embodiments, the compositions of the disclosure may include point mutations in the binding region that decrease receptor and matrix binding. In some embodiments, the compositions of the disclosure may include point mutations in the binding region that modulate receptor binding and matrix binding, independently. For example, in some embodiments, the compositions of the disclosure may include point mutations in the binding region that increases receptor binding and decrease matrix binding.

In some embodiments, the polypeptides of the disclosure may include mutations that impact the propensity of polypeptides to aggregate. Mutations may include for example, F275A (SEQ ID NO: 846), W282R (SEQ ID NO: 847), V287A (SEQ ID NO: 848), L297Q (SEQ ID NO: 849), and V305E (SEQ ID NO: 850) of APOE3 (SEQ ID NO: 1).

In some embodiments, polypeptides of the disclosure may include one or more heparin binding and/or heparin sulfate proteoglycan binding region. The heparin binding region may be derived from a parent protein such as but not limited to an apolipoprotein e.g., APOE. Heparin binding regions in APOE may include residues in the vicinity of residues 142-147, 160-165, 243-272, 261-290 of SEQ ID NO: 1-3 (Weisgraber et al. J Biol Chem. 1986; 261:2068-2076; the contents of which are herein incorporated by reference in its entirety).

In some embodiments, polypeptides of the disclosure may include one or more glycosylation site, which herein refers to a site in a polypeptide of the disclosure that may be enzymatically modified to include a carbohydrate moiety. In some embodiments, the apolipoprotein variant may include one or more mutations that increase glycosylation of the polypeptide. Non-limiting example includes F275A of APOE3 (SEQ ID NO:1), APOE2 (SEQ ID NO:2), or APOE4 (SEQ ID NO:3). In some embodiments, apolipoprotein variant may include one or more mutations to decrease glycosylation of the polypeptide. For example, residue 212 of SEQ ID NO: 1 (SEQ ID NO: 855), residue 212 of SEQ ID NO:2 (SEQ ID NO: 856), or residue 212 of SEQ ID NO: 3 (SEQ ID NO: 857) may be mutated to alanine prevent its glycosylation.

In some embodiments, the polypeptides of the disclosure further include an affinity tag. Non-limiting examples of affinity tag include HHHHHH (SEQ ID NO: 852), WSHPQFEK (SEQ ID NO: 853), and/or DYKDDDDK (SEQ ID NO: 854). The term “affinity tag,” as used herein, refers to a peptide sequence appended to a protein, so that the protein can be purified from its crude biological source using an affinity technique. Examples of affinity tags include, but are not limited to, chitin binding protein (CBP), maltose binding protein (MBP), FLAG tag, poly(His) tag, Strep-tag and glutathione-S-transferase (GST). In some embodiments, the affinity tag is removable by chemical agents or by enzymatic means, such as proteolysis or intein splicing.

### Structured Sequence Region

In some embodiments, the polypeptides of the disclosure may include one or more structured sequence regions. As used herein, “structured sequence region” refers to a region of a polypeptide containing one or more folding patterns in its backbone formed by the stable arrangement of amino acid residues of the polypeptide. The folding pattern may be an alpha helix, a beta confirmation, beta turn or a combination thereof. In some embodiments, the structured sequence region of the polypeptide may be derived from one or more parent proteins. Non-limiting examples of the parent protein include, APOE, APOA, APOB, APOC, APOD, APOF, APOH, APOJ, APOL, APOM, or APOO. In some embodiments, the polypeptide includes structured sequence regions from a plurality (e.g., a combination) of parent proteins.

The structured sequence region (SSR) may be or may include an alpha helix sequence region. As used herein, alpha helix sequence refers to a type of a structure in which the polypeptide backbone is tightly wound around an imaginary axis drawn longitudinally through the middle and the R groups of the amino acids protrude outward from the backbone. In this confirmation, every backbone N-H group forms a hydrogen bond with the backbone C═O group of the amino acid located four residues earlier along the protein sequence.

In some embodiments, the alpha helix sequence region may be or may include one or more amphipathic alpha helix sequence regions. An amphipathic (or amphiphilic) helix is used to refer an α-helix may include both hydrophobic and hydrophilic amino acid residues arranged in such a way as to create two faces on opposite sides of the helix, one face being hydrophobic while the other is hydrophilic. In some embodiments, the amino acid sequence of an amphiphilic helix may include a hydrophilic amino acid at every second or third position in the sequence. The amphipathic alpha helices described herein may also include amino acids with hydrophobic side chains at every third or fourth position.

In some embodiments, the SSR may include a helix bundle. As used herein, the term “helix bundle” refers to a protein fold composed of several alpha helices that may be or may nearly be parallel or antiparallel to each other.

SSRs of the disclosure may include one or more peptides. The SSR peptides may be of an any length for e.g., from about 2-55 amino acids long. In some embodiments, the peptides within the SSR may be 11 amino acids (herein a “11-mer”) or multiples thereof such as, but not limited to, 22-mer, 33-mer, 44-mer, or 55-mer. In some embodiments, SSR peptides may be or may be a part of an amphipathic alpha helix sequence region. In some embodiments, the polypeptides of the disclosure may include from about 5 to about 30 SSR peptides. The SSR peptides (SSRPs) may be derived from the same parent proteins or the polypeptides of the disclosure may include SSR peptides derived from 2 or more parent proteins. In some embodiments, one or more of the SSRPs may be repeated in the polypeptides of the disclosure. In some embodiments, point mutations impacting aggregation propensity, glycosylation, receptor binding, or other properties are introduced. In some embodiments, a linker region between lipid-binding α-helices may be introduced.

In some embodiments, the SSRPs are not naturally-occurring. In some embodiments, the SSRPs may include a hydrophobic face which engages with lipids and a polar face with charged residues facilitating solubility in aqueous media and electrostatic interactions between within the lipoprotein systems. In some embodiments, positions 2, 3, 6, and 10 within the 11-mer may be a hydrophobic residue, such as Leucine. In some embodiments positions 4, 5, 7, 8, and 9 may be polar or charged residues, such as Glutamine or Arginine. In some embodiments, position 1 or position 11 are a helix breaking residue, such as Pro or Gly. In some embodiments, SSRPs are shorter or longer than 11 residues.

Table 2 provides non-limiting examples of SSRP sequences.

TABLE 2
Structured sequence region peptides

| | Protein | SEQ ID |
|---|---|---|
| SSRP ID | sequence | NO: |
| SSRP1 | ALMDETMKELK | 11 |
| SSRP2 | AYKSELEEQLT | 12 |
| SSRP3 | PVAEETRARLS | 13 |
| SSRP4 | KELQAAQARL | 14 |
| | G | |
| SSRP5 | ADMEDVCGRL | 15 |
| | V | |
| SSRP6 | QYRGEVQAML | 16 |
| | G | |
| SSRP7 | QSTEELRVRLA | 17 |
| SSRP8 | SHLRKLRKRLL | 18 |
| SSRP9 | RDADDLQKRL | 19 |
| | A | |
| SSRP10 | VYQAGAREGA | 20 |
| | E | |
| SSRP11 | RGLSAIRERLG | 21 |
| SSRP12 | DRLDEVKEQV | 22 |
| | A | |
| SSRP13 | AKLEEQAQQIR | 23 |
| SSRP14 | LQAEAFQARLK | 24 |
| SSRP15 | PLVEDMQRQW | 25 |
| | A | |
| SSRP16 | GLVEKVQAAV | 26 |
| | G | |
| SSRP17 | QSTEELRVSLA | 27 |
| SSRP18 | PLAEDMQRQW | 28 |
| | A | |
| SSRP19 | GQVEKVQAAE | 29 |
| | G | |
| SSRP20 | GQVEKVQAAV | 30 |
| | G | |
| SSRP21 | ADMEDVRGRL | 31 |
| | V | |
| SSRP22 | RDADDLQKCL | 32 |
| | A | |
| SSRP23 | QSTEELRVXLA | 33 |
| SSRP24 | RDADDLQKXLA | 34 |
| SSRP25 | DRLDEVKEQV | 35 |
| | X | |
| SSRP26 | LQAEAXQARLK | 36 |
| SSRP27 | GXVEKVQAAX | 37 |
| | G | |
| SSRP28 | PLQERAQ | 38 |
| SSRP29 | AWGERLRAR | 39 |
| SSRP30 | MEEMGSRTR | 40 |
| SSRP31 | LKLLDNWDSVT | 41 |
| SSRP32 | STFSKLREQLG | 42 |
| SSRP33 | PVTQEFWDNL | 43 |
| | E | |
| SSRP34 | KETEGLRQEM | 44 |
| | S | |
| SSRP35 | KDLEEVKAKVQ | 45 |
| SSRP36 | PYLDDFQKKW | 46 |
| | Q | |
| SSRP37 | EEMELYRQKV | 47 |
| | E | |
| SSRP38 | PLRAELQEGAR | 48 |
| SSRP39 | QKLHELQEKLS | 49 |
| SSRP40 | PLGEEMRDRA | 50 |
| | R | |
| SSRP41 | AHVDALRTHLA | 51 |
| SSRP42 | PYSDELRQRLA | 52 |
| SSRP48 | SALEEYTKKLN | 58 |
| SSRP49 | ALFQDKLGEVN | 59 |
| SSRP50 | TYAGDLQKKLV | 60 |
| SSRP51 | PFATELHERLA | 61 |
| SSRP52 | KDSEKLKEEIG | 62 |
| SSRP53 | KELEELRARLL | 63 |
| SSRP54 | PHANEVSQKIG | 64 |
| SSRP55 | DNLRELQQRLE | 65 |
| SSRP56 | PYADQLRTQV | 66 |
| | N | |
| SSRP57 | TQAEQLRRQLT | 67 |
| SSRP58 | PYAQRMERVL | 68 |
| | R | |
| SSRP59 | ENADSLQASLR | 69 |
| SSRP60 | PHADELKAKID | 70 |
| SSRP61 | QNVEELKGRLT | 71 |
| SSRP62 | PYADEFKVKID | 72 |
| SSRP63 | QTVEELRRSLA | 73 |
| SSRP64 | PYAQDTQEKL | 74 |
| | N | |
| SSRP65 | HQLEGLTFQM | 75 |
| | K | |
| SSRP66 | KNAEELKARIS | 76 |
| SSRP67 | ASAEELRQRLA | 77 |
| SSRP68 | PLAEDVRGNLR | 78 |
| SSRP69 | QQVEEFRRRV | 79 |
| | E | |
| SSRP70 | PYGENFNKALV | 80 |
| SSRP71 | QQMEQLRQKL | 81 |
| | G | |
| SSRP72 | PHAGDVEGHL | 82 |
| | S | |
| SSRP73 | FLEKDLRDKVN | 83 |
| SSRP74 | PLXEELXXRLX | 84 |
| SSRP75 | PLXEEXRXXLX | 85 |
| SSRP76 | XXLEELREKLX | 86 |
| SSRP77 | XXXEEXQXRLX | 87 |
| SSRP78 | LQAEAAQARLK | 88 |
| SSRP79 | GLVEKVQAAE | 89 |
| | G | |
| SSRP80 | DRLDEVKEQE | 90 |
| | A | |
| SSRP81 | ELRVRLA | 91 |
| SSRP82 | CGRLV | 92 |
| SSRP83 | PLVEDM | 93 |
| SSRP84 | VRLA | 94 |
| SSRP85 | ARLKENGG | 95 |
| SSRP86 | PYSDELROCLA | 96 |
| SSRP87 | RDADDLQCRL | 97 |
| | A | |
| SSRP88 | VLMEDTMTEV | 858 |
| | K | |
| SSRP89 | AYKKELEEQLG | 859 |
| SSRP90 | PVAEETRARLA | 860 |
| SSRP91 | KEVQAAQARL | 861 |
| | G | |
| SSRP92 | ADMEDLRNRL | 862 |
| | G | |
| SSRP93 | QYRNEVNTML | 863 |
| | G | |
| SSRP94 | QSTEELRSRLS | 864 |
| SSRP95 | THLRKMRKRL | 865 |
| | M | |
| SSRP96 | VYKAGAQEGA | 866 |
| | E | |
| SSRP97 | RGVSAIRERLG | 867 |
| SSRP98 | DRLEEVREQM | 868 |
| | E | |
| SSRP99 | SKMEEQTQQIR | 869 |
| SSRP100 | LQAEIFQARIK | 870 |
| SSRP101 | NLMEKIQASVA | 871 |

Polypeptides of the disclosure may include a plurality of amphipathic α-helices covalently linked together using well-known and established fragment conjugation or ligation chemistries described by Dawson and Kent in their comprehensive reviewed “Synthesis of Native Proteins by Chemical Ligation” 2000, Ann Rev Biochem 69:923-60 (the contents of which are herein incorporated by reference in its entirety). In some embodiments ligation of the helices and the linker segments can be performed—as described in “Synthesis of Native Proteins by Chemical Ligation” 2000, Ann Rev Biochem 69:923-60. α-helices may be separated by a short linker sequence which may be provided by a sequence of natural or non-natural amino acids. In some embodiments, the linker sequence can form an amino acid loop.

In some embodiments, the SSR may be or may include a beta confirmation sequence region. As used herein, a “beta confirmation sequence region” refers to a type of structure in which the backbone of the polypeptide is extended into a zigzag pattern rather than a helical structure. In some embodiments, the zigzag polypeptide chains can be arranged side by side to form a beta sheet. The zigzag polypeptide chains can be arranged side by side to form a structure resembling a series of pleats. Hydrogen bonds are formed between adjacent segments of polypeptide chain. The individual segments that form a β sheet are usually nearby on the polypeptide chain but can also be quite distant from each other in the linear sequence of the polypeptide; they may even be segments in different polypeptide chains. In some embodiments, the adjacent polypeptide chains in a β sheet can be either parallel or antiparallel (having the same or opposite amino-to-carboxyl orientations, respectively). The β-sheet domains may be fundamental in establishing strong bonds to the lipids of the lipoprotein systems of the disclosure. In some embodiments, one or more amphipathic α-helix domain may be located between the two β-sheet domains.

In some embodiments, the SSR may be or may include a beta turn sequence region. As used herein, “a beta turn sequence region” refers to a type of structure which connects the ends of two adjacent segments of a beta sheet.

In some embodiments, the structured sequence region of the disclosure may include one or more folding patterns which are in a configuration such as ABABAB or AABBAABBAABB or ABCABCABC or variants thereof repeated once, twice, or more than three times. In these patterns, each letter, A, B, or C represent a different folding pattern. In yet another embodiment, the polypeptides of the disclosure may include two or more folding patterns component sequences with each component having one or more sequences. As a non-limiting example, the sequences may be in a pattern such as ABABAB or AABBAABBAABB or ABCABCABC or variants thereof repeated once, twice, or more than three times in each of the regions.

In some embodiments, the SSR may include one or more gap sequences adjacent to folding patterns present within. The gap sequence may connect adjacent folding patterns within the SSR.

In some embodiments, the polypeptides of the disclosure may include at least 1, 2, 3, 4, 5, 6, 7, 8, 9, 10, 11, 12, 13, 14, 15, 16, or 17 SSRPs. In some embodiments, the structured sequence region peptides may include 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids (10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100-mer).

In some embodiments, the structured sequence region includes hydrophobic residues on its inner face. In some embodiments, the hydrophobic residues on the inner face of the structured sequence region bind lipid acyl chains. In some embodiments, the structured sequence region includes endogenous hydrophobic residues on its inner face. In some embodiments, the structured sequence region includes substitutions of any amino acids with hydrophobic amino acids to have hydrophobic residues on its inner face. In some embodiments, the structured sequence region includes substitutions of any amino acids with hydrophobic amino acids to increase hydrophobic residues on its inner face. In some embodiments, the structured sequence region includes substitutions of any hydrophilic or neutral amino acids with hydrophobic amino acids to have hydrophobic residues on its inner face. In some embodiments, the structured sequence region includes substitutions of any hydrophilic or neutral amino acids with hydrophobic amino acids to increase hydrophobic residues on its inner face.

In some embodiments, the structured sequence region includes hydrophilic residues on its outer face. In some embodiments, the hydrophilic residues on the outer face of the structured sequence region solubilize the lipoprotein particle in aqueous media or solution.

In some embodiments, the structured sequence region includes endogenous hydrophilic residues on its outer face. In some embodiments, the structured sequence region includes substitutions of any amino acids with hydrophilic amino acids to have hydrophilic residues on its outer face. In some embodiments, the structured sequence region includes substitutions of any amino acids with hydrophilic amino acids to increase hydrophilic residues on its outer face. In some embodiments, the structured sequence region includes substitutions of any hydrophobic or neutral amino acids with hydrophilic amino acids to have hydrophilic residues on its outer face. In some embodiments, the structured sequence region includes substitutions of any hydrophobic or neutral amino acids with hydrophilic amino acids to increase hydrophilic residues on its outer face.

In some embodiments, the structured sequence region includes highly charged residues on its top or bottom faces. In some embodiments, the highly charged residues are, for example, Arg or Glu. In some embodiments, the interaction between the top or bottom faces of adjacent peptides of the structured sequence region is an intramolecular interaction. In some embodiments, the interaction between the top or bottom faces of adjacent peptides of the structured sequence region is an intermolecular interaction.

In some embodiments, the structured sequence region includes endogenous highly charged residues on its top or bottom faces. In some embodiments, the structured sequence region includes endogenous, e.g., Arg or Glu residues on its top or bottom faces. In some embodiments, the structured sequence region includes substitutions of any amino acids with highly charged amino acids to have highly charged residues on its top or bottom faces. In some embodiments, the structured sequence region includes substitutions of any amino acids with highly charged amino acids to increase highly charged residues on its top or bottom faces. In some embodiments, the structured sequence region includes substitutions of any neutral amino acids with highly charged amino acids to have highly charged residues on its top or bottom faces. In some embodiments, the structured sequence region includes substitutions of any neutral amino acids with highly charged amino acids to increase hydrophilic residues on its outer face.

In some embodiments, the structured sequence region includes substitutions of any amino acids with, e.g., Arg or Glu to have highly charged residues on its top or bottom faces. In some embodiments, the structured sequence region includes substitutions of any amino acids with, e.g., Arg or Glu to increase highly charged residues on its top or bottom faces. In some embodiments, the structured sequence region includes substitutions of any neutral amino acids with, e.g., Arg or Glu to have highly charged residues on its top or bottom faces. In some embodiments, the structured sequence region includes substitutions of any neutral amino acids with, e.g., Arg or Glu to increase hydrophilic residues on its outer face.

In some embodiments, the residues on the top or bottom faces of the structured sequence region are substituted with natural or non-natural amino acids. In some embodiments, the residues on the top or bottom faces of the structured sequence region are substituted with a natural or non-natural amino acid. In some embodiments, the residues on the top or bottom faces of the structured sequence region are substituted with Cys or Tyr. In some embodiments, the substituted residues on the top or bottom faces of the structured sequence region crosslink multiple molecules via covalent bonds or non-covalent bonds. In some embodiments, the covalent bond is a disulfide bond. In some embodiments, the structured sequence region provides the structural components for lipid binding. In some embodiments the structured sequence region binds ATP-binding cassette transporters (ABC transporters) and stimulates cholesterol efflux. In some embodiments, the structured sequence region serves as the sites for chemical crosslinking. In some embodiments, the polypeptides of the disclosure form a dimer. In some embodiments, the polypeptides form a homodimer. In some embodiments, the polypeptides form a disulfide homodimer. In some embodiments, the polypeptides form a dimer by utilizing a protein dimerizer. The term “protein dimerizer,” also known as chemical inducers of dimerization, as used herein, refers to a chemical compound that binds two different proteins and brings them into close proximity solely in the presence of the dimerizer. The protein dimerizer is commonly used to build protein complexes. Exemplary protein dimerizers include, but are not limited to, FK1012 (a derivative of tacrolimus) for an FKBP (FK506 binding protein) homodimer, FK506 (tacrolimus) for an FKBP and Calcineurin A (CNA) dimer, FKCsA for a FKBP and CyP-Fas dimer, rapamycin for an FKBP and FRB (FKBP-rapamycin-binding) domain of mTOR dimer, Coumermycin for a GyrB (DNA gyrase subunit B) homodimer, Gibberellin for a GAI (a member of the DELLA proteins) and GID1 (gibberellin insensitive dwarf 1) dimer, HaXS for a SNAP-tag and HaloTag dimer, TMP-HTag for an eDHFR (Dihydrofolate reductase) and HaloTag dimer, and ABT-737 for a Bcl-xL and Fab (AZ1) dimer. In some embodiments, the non-natural means include light-induced dimerization. Exemplary light-induced dimerization include, but are not limited to, use of photosensitive proteins or a photocaged chemical dimerizer.

In some embodiments, the first or the last residue in SSRP is a helix breaking residue, thereby allowing flexibility between sequential helices. In some embodiments, the helix breaking residue is Pro or Gly. In some embodiments, the SSRPs stimulate ABCA (ATP-binding Cassette Transporter A)/ABCG (ATP-binding cassette transporters G)-mediated cholesterol efflux. In some embodiments, the SSRPs may be sufficient to stimulate ABCA/ABCG-mediated cholesterol efflux.

In some embodiments, the SSRPs may be derived from alignments of apolipoprotein consensus repeat units. In some embodiments, the apolipoprotein consensus repeat units may be combined in a non-natural order. In some embodiments, the apolipoprotein consensus repeat units may be derived from sequences of a non-human APOE. In some embodiments, the apolipoprotein consensus repeat units may be derived from the sequences of other exchangeable apolipoproteins (e.g., ApoA1 or ApoA-1). In some embodiments, the apolipoprotein consensus repeat units may be derived from the sequences of ApoA1 or ApoA-1.

In some embodiments, the structured sequence region may include point mutations or chemical crosslinking to alter the accessibility and effective affinity of the binding region. In some embodiments, the structured sequence region may include point mutations introducing additional Arg residues to alter the aggregation propensity and protease stability of the protein, as well as its half-life in endosomal/lysosomal compartments. In some embodiments, the structured sequence region may include point mutations to alter the multimerization propensity of the polypeptides in solution.

### N-Terminal Sequence Region

In some embodiments, the polypeptides of the disclosure may include an N-terminal sequence region. The N-terminal sequence region may be derived from any parent protein or polypeptide. In some aspects, the N terminal sequence region is derived from an apolipoprotein (herein, “apolipoprotein N-terminal sequence region”). As a non-limiting example, apolipoprotein may be APOE, APOA, APOB, APOC, APOD, APOF, APOH, APOJ, APOL, APOM, or APOO. In some embodiments, the N-terminal sequence region may include a region or a fragment of the terminal region of the parent protein or polypeptide. In some embodiments, the N-terminal sequence region may include a region or a fragment of the N-terminus (terminated by an amino acid with a free amino group (NH2)) of the parent protein or polypeptide. In some embodiments, N-terminal regions may include any length of amino acids that includes the N-terminus but does not include the C-terminus of the parent protein or polypeptide. For example, the N-terminal sequence region may include 1-10, 5-15, 10-20, 15-25, 20-30, 25-35, 30-40,35-45, 40-50,45-55, 50-60,55-65, 60-70,65-75, 70-80,75-85, 80-90,85-95, 90-100, 95-105, 100-110, 105-115, 110-120, 115-125, 120-130, 125-135, 130-140, 135-145,140-150, 145-155, 150-160, 155-165, 160-170, 165-175, 170-180, 175-185, 180-190, 185-195, 190-200, 195-205, 200-210, 205-215, 210-220, 215-225, 220-230, 225-235, 230-240, 235-245, 240-250, 245-255, 250-260, 255-265, 260-270, 265-275, 270-280, 275-285, 280-290, 285-295, 290-300, 295-305, 300-310, 305-315, 310-320, 315-325, 320-330, 325-335, 330-340, 335-345, 340-350, 345-355, 350-360, 355-365, 360-370, 365-375, 370-380, 375-385, 380-390, 385-395, 390-400, 400-500, 500-1000, 1000-5000 from the N-terminus of any parent protein or polypeptide.

In some embodiments, the N terminal sequence region may be APOE N terminal sequence region or APOA1 N terminal sequence region. Table 3 provides non-limiting examples N terminal sequence regions. The N terminal sequence region may include additional or fewer amino acids than any of those described in Table 3. Such amino acid sequences may include about 1 more or fewer amino acids, about 2 more or fewer amino acids, about 3 more or fewer amino acids, about 4 more or fewer amino acids, about 5 more or fewer amino acids, about 6 more or fewer amino acids, about 7 more or fewer amino acids, about 8 more or fewer amino acids, about 9 more or fewer amino acids, about 10 more or fewer amino acids or greater than 10 more or fewer amino acids.

TABLE 3
N terminal sequence regions

| | | SEQ ID |
|---|---|---|
| NTERM ID | Protein sequence | NO: |
| NTERM1 | KVEQAVETEPEPELRQQTEWQ | 98 |
| | SGQRWELALGRFWDYLRWVQT | |
| | LSEQVQEELLSSQVTQELR | |
| NTERM2 | DEPPQSPWDRVKDLATVYVDV | 99 |
| | LKDSGRDYVSQFEGSALGKQL | |
| | N | |
| NTERM3 | GQRWELALGRFWDYLRWVQTL | 100 |
| | SEQVQEELLSSQVTQELR | |
| NTERM4 | EQVQEELLSSQVTQELR | 101 |
| NTERM5 | QVTQELR | 102 |
| NTERM6 | RLS | — |
| NTERM7 | EGELEVTDQLPGQSDQPWEQA | 872 |
| | LNRFWDYLRWVQTLSDQVQEE | |
| | LQSSQVTQELT | |

### C Terminal Sequence Region

In some embodiments, the polypeptides of the disclosure may include a C-terminal sequence region. The C-terminal sequence region may be derived from any parent protein or polypeptide. In some aspects, the C terminal sequence region is derived from an apolipoprotein. As a non-limiting example, apolipoprotein may be APOE, APOA, APOB, APOC, APOD, APOF, APOH, APOJ, APOL, APOM, or APOO (herein, “apolipoprotein C-terminal sequence region”). In some embodiments, the C-terminal sequence region may include a region or a fragment of the terminal region of the parent protein or polypeptide. In some embodiments, the C-terminal sequence region may include a region or a fragment of the C-terminus (terminated by an amino acid with a carboxy group (NH2)) of the parent protein or polypeptide. In some embodiments, C-terminal regions may include any length of amino acids that includes the C-terminus, but does not include the N-terminus of the parent protein or polypeptide. For example, the C-terminal sequence region may include 1-10, 5-15, 10-20, 15-25, 20-30, 25-35, 30-40,35-45, 40-50,45-55, 50-60,55-65, 60-70,65-75, 70-80,75-85, 80-90,85-95, 90-100, 95-105,100-110,105-115,110-120,115-125,120-130,125-135, 130-140,135-145,140-150, 145-155, 150-160, 155-165, 160-170, 165-175, 170-180, 175-185, 180-190, 185-195, 190-200, 195-205, 200-210, 205-215, 210-220, 215-225, 220-230, 225-235, 230-240, 235-245, 240-250, 245-255, 250-260, 255-265, 260-270, 265-275, 270-280, 275-285, 280-290, 285-295, 290-300, 295-305, 300-310, 305-315, 310-320, 315-325, 320-330, 325-335, 330-340, 335-345, 340-350, 345-355, 350-360, 355-365, 360-370, 365-375, 370-380, 375-385, 380-390, 385-395, 390-400, 400-500, 500-1000, 1000-5000 from the C-terminus of any parent protein or polypeptide.

In some embodiments, the C terminal sequence region may be APOE C terminal sequence region, or APOA1 C terminal sequence region. Table 4 provides non-limiting examples C terminal sequence regions. The C terminal sequence region may be any of these sequences in Table 4, or include additional or fewer amino acids than those listed. Such amino acid sequences may include about 1 more or fewer amino acids, about 2 more or fewer amino acids, about 3 more or fewer amino acids, about 4 more or fewer amino acids, about 5 more or fewer amino acids, about 6 more or fewer amino acids, about 7 more or fewer amino acids, about 8 more or fewer amino acids, about 9 more or fewer amino acids, about 10 more or fewer amino acids or greater than 10 more or fewer amino acids.

TABLE 4
C terminal sequence regions

| CTERM ID | Protein sequence | SEQ ID NO: |
|---|---|---|
| CTERM1 | TSAAPVPSDNH | 103 |
| CTERM2 | TQ | — |
| CTERM3 | TNSIASTTVPLENQ | 873 |

### Linkers

In some embodiments, the lipoprotein systems of the disclosure may include a linker. As used herein, the term linker refers to any molecule or group of molecules that connect two molecules or two parts of a molecule such as two regions of a polypeptide. In some embodiments, the linker is a peptidyl linker. In some embodiments, the peptidyl linker includes a short peptide of 1 to about 25 amino acids. In some embodiments, the peptidyl linker includes 1, 2, 3, 4, 5, 6, 7, 8, 9, 10, 12, 13, 14, 15, 16, 17, 18, 19, 20, 21, 22, 23, 24, or 25 residues. In some embodiments, the peptidyl linker includes glycine and serine. In some embodiments, the residues of the peptidyl linker are glycine or serine. In some embodiments, the residues of the peptidyl linker are glycine and serine. In some embodiments, the peptidyl linker may be rich in glycine for flexibility, as well as serine or threonine for solubility.

In some embodiments, the linker is a chemical linker. In some embodiments, the chemical linker is a covalent or noncovalent linker. In some embodiments, the chemical linker includes a chemically reactive functional group to react with a second chemically reactive functional group, thereby forming a covalent linker. In some embodiments, the chemical linker includes the resulting linker formed by reacting two reactive groups (moieties), e.g., a covalent reactive group (e.g., alkyne, thiol, azide, maleimide). In some embodiments, the chemical linker is polyvalent and/or formed by conjugate chemistry techniques. Examples of the chemical linker include, but are not limited to, —O—, —S—, —C(O)—, —C(O)O—, —C(O)NH—, —S(O)2NH—, —NH—, —NHC(O)NH—.

In some embodiments, the linker includes a linear structure. In some embodiments, the linker includes a non-linear structure. In some embodiments, the linker includes a branched structure. In some embodiments, the linker includes a cyclic structure.

In some embodiments, the linker conjugates or links multiple polypeptides or fragments of polypeptides or compounds. In some embodiments, the linker binds to two polypeptides or compounds. In some embodiments, the linker binds to three polypeptides or compounds.

In some embodiments, “Linker” (L) or “linker domain” or “linker region” or “linker module” or “peptide linker” as used herein refers to an oligo- or polypeptide region of from about 1 to 100 amino acids in length, which links together any of the domains/regions of the polypeptide (also called peptide linker). The peptide linker may be 1-40 amino acids in length, or 2-30 amino acids in length, or 20-80 amino acids in length, or 50-100 amino acids in length. Linker length may also be optimized depending on the type of polypeptide or payloads utilized and based on the crystal structure of the polypeptide or payload. In some instances, a shorter linker length may be preferably selected. In some aspects, the peptide linker is made up of amino acids linked together by peptide bonds, preferably from 1 to 20 amino acids linked by peptide bonds, wherein the amino acids are selected from the 20 naturally occurring amino acids: Glycine (C), Alanine (A), Valine (V), Leucine (L), Isoleucine (S), Serine (S), Cysteine (C), Threonine (T), Methionine (M), Proline (P), Phenylalanine (F), Tyrosine (Y), Tryptophan (W), Histidine (H), Lysine (K), Arginine (R), Aspartate (D), Glutamic acid (E), Asparagine (N), and Glutamine (Q). One or more of these amino acids may be glycosylated, as is understood by those in the art. In some aspects, amino acids of a peptide linker may be selected from Alanine (A), Glycine (C), Proline (P), Asparagine (R), Serine (S), Glutamine (Q) and Lysine (K).

In one example, an artificially designed peptide linker may preferably be composed of a polymer of flexible residues like Glycine (C) and Serine (S) so that the adjacent protein domains are free to move relative to one another. Longer linkers may be used when it is desirable to ensure that two adjacent domains do not interfere with one another. The choice of a particular linker sequence may concern if it affects biological activity, stability, folding, targeting and/or pharmacokinetic features of the fusion construct.

A linker sequence may be a natural linker derived from a multi-domain protein. A natural linker is a short peptide sequence that separates two different domains or motifs within a protein. For example, the linker sequence may be a hinge sequence derived from an apolipoprotein or a variant thereof. Table 5 provides non-limiting examples of linkers useful in the present disclosure.

TABLE 5
Linkers

| | Linker/Human protein | | SEQ |
|---|---|---|---|
| | from human multi- | | ID |
| ID | domain proteins | Sequence | NO: |
| LINK1 | Flexible G/S rich linker | G | — |
| LINK2 | Flexible G/S rich linker | S | — |
| LINK3 | Flexible G/S rich linker | GG | — |
| LINK4 | Flexible G/S rich linker | GGG | — |
| LINK5 | Flexible G/S rich linker | GGGG | 104 |
| LINK6 | Flexible G/S rich linker | GGGGGG | 105 |
| LINK7 | GGS₂ | GGSGGS | 106 |
| LINK8 | GGS₃ | GGSGGSGGS | 107 |
| LINK9 | GGS₄ | GGSGGSGGSGGS | 108 |
| LINK10 | Flexible G/S rich linker | GGGGGGGG | 109 |
| LINK11 | Linker (GS); BamH1 Site | GS | — |
| LINK12 | Linker (GGS); Flexible G/S rich linker; | GGS | — |
| | ACRIFLAVINE RESISTANCE PROTEIN B | | |
| LINK13 | Flexible G/S rich linker | GGGS | 110 |
| LINK14 | Flexible G/S rich linker | GGGGS | 111 |
| LINK15 | Flexible G/S rich linker | GGGGSGGGGS | 112 |
| LINK16 | Linker ((G4S)3); | GGGGSGGGGSGGGGS | 113 |
| | Flexible G/S rich linker | | |
| LINK17 | Flexible G/S rich linker | GGGGSGGGGSGGGGSGG | 114 |
| | | GGS | |
| LINK18 | Flexible G/S rich linker | GGSGGGSEGGGSEGGGSE | 115 |
| | | GGGSEGGGSEGGGSGGGS | |
| LINK19 | Serine rich linker S4G | SSSSG | 116 |
| LINK20 | Serine rich linker | SSSSGSSSSG | 117 |
| LINK21 | Serine rich linker | SGSSSSGSSSSGS | 118 |
| LINK22 | Serine rich linker (S4G)3 | SSSSGSSSSGSSSSG | 119 |
| LINK23 | Serine rich linker | SVTVSSSGSSSSGSSSSGS | 120 |
| LINK24 | Serine rich linker (S4G)4 | SSSSGSSSSGSSSSGSSSS | 121 |
| | | G | |
| LINK25 | Serine rich linker (S4G)5 | SSSSGSSSSGSSSSGSSSS | 122 |
| | | GSSSSG | |
| LINK26 | Artificial linker | EFSTEF | 123 |
| LINK27 | Artificial linker | GGSGG | 124 |
| LINK28 | Artificial linker | GGSG | 125 |
| LINK29 | Artificial linker | GGSGGG | 126 |
| LINK30 | Artificial linker | SGGGSGGGGSGGGGSGG | 127 |
| | SG3-(SG4)3-SG3-SLQ | GGSGGGSLQ | |
| LINK31 | Artificial linker SG3S | SGGGS | 128 |
| LINK32 | Linker SG3-(SG4)3S | SGGGSGGGGSGGGGSGG | 129 |
| | | GGS | |
| LINK33 | Artificial linker | GGSGGGSGG | 130 |
| LINK34 | Artificial linker | GGGGG | 131 |
| LINK35 | Artificial linker (G4S)5 | GGGGSGGGGSGGGGSGG | 132 |
| | | GGSGGGGS | |
| LINK36 | Artificial linker (G4S)6 | GGGGSGGGGSGGGGSGG | 133 |
| | | GGSGGGGSGGGGS | |
| LINK37 | Artificial linker (S4G)2 | SSSSGSSSSGG | 134 |
| LINK38 | Artificial linker (S4G)6 | SSSSGSSSSGSSSSGSSSS | 135 |
| | | GSSSSGSSSSG | |
| LINK39 | Linker SG3-(SG4)5-SG3-S | SGGGSGGGGSGGGGSGG | 136 |
| | | GGSGGGGSGGGGSGGGS | |
| LINK40 | Flexible G/S rich linker | GGSAGGSGSGSSGGSSGA | 137 |
| | | SGTGTAGGTGSGSGTGSG | |
| LINK41 | Artificial linker | GKSSGSGSESKS | 138 |
| LINK42 | Artificial linker | GGSTSGSGKSSEGKG | 139 |
| LINK43 | Artificial linker | GSTSGSGKSSSEGSGSTKG | 140 |
| LINK44 | Artificial linker | VDYPYDVPDYALD | 141 |
| LINK45 | Artificial linker | EGKSSGSGSESKEF | 142 |
| LINK46 | Linker | SGGGSGGGGSGGGGSGG | 143 |
| | | GGSGGGSLQYPYDVPDYA | |
| LINK47 | Linker | DYKDDDDK | 144 |
| LINK48 | Linker | SGGGSGGGGSGGGGSGG | 145 |
| | | GGSYPYDVPDYASGGGS | |
| LINK49 | Linker | GSGATNFSLLKQAGDVEEN | 146 |
| | | PGP | |
| LINK50 | Linker | DVEXNPGP | 147 |
| LINK51 | Linker | DIEXNPGP | 148 |
| LINK52 | Linker (SG4)3SG3SLQ | SGGGGSGGGGSGGGGSG | 149 |
| | | GGSLQ | |
| LINK53 | Linker (GSG) (BamH1-Gly) | GSG | — |
| LINK54 | Linker | QLIGMLQGLMRDL | 150 |
| LINK55 | Linker | ASFE | 151 |
| LINK56 | Linker | EF | — |
| LINK57 | Linker (TS)/Spel Site | TS | — |
| LINK58 | Linker | HM | — |
| LINK59 | Linker | MH | — |
| LINK60 | Artificial linker | PGS | — |
| LINK61 | Artificial linker | ATK | — |
| LINK62 | Artificial linker | ASK | — |
| LINK63 | Artificial linker | ATKASK | 152 |
| LINK64 | Artificial linker | ATKGATK | 153 |
| LINK65 | Artificial linker | IAKAFKN | 154 |
| LINK66 | Artificial linker | VRGSPAINVAVHVF | 155 |
| LINK67 | Artificial linker | GSTSGSGKPGSGEGSTKG | 156 |
| LINK68 | Flexible G/S rich linker | KESGSVSSEQLAQFRSLD | 157 |
| LINK69 | Cleavable Disulfide | GFLG | 158 |
| LINK70 | Cleavable Disulfide | PLGLWA | 159 |
| LINK71 | Cleavable Disulfide | RVLAEA | 160 |
| LINK72 | Cleavable Disulfide | GGIEGRGS | 161 |
| LINK73 | Cleavable Disulfide | RRRRRRRRR | 162 |
| LINK74 | Cleavable Disulfide | EDVVCCSMSY | 163 |
| LINK75 | Cleavable Disulfide | TRHRQPRGWE | 164 |
| LINK76 | Cleavable Disulfide | AGNRVRRSVG | 165 |
| LINK77 | Cleavable Disulfide | CRRRRRREAEAC | 166 |
| LINK78 | Cleavable Disulfide | VSQTSKLTRAETVFPDV | 167 |
| LINK79 | Cleavable Disulfide | LEAGCKNFFPRSFTSCGSLE | 168 |
| LINK80 | Flexible G/S rich linker | GSAGSAAGSGEF | 169 |
| LINK81 | Flexible G/S rich linker | EGKSSGSGSESKST | 170 |
| LINK82 | Flexible G/S rich linker | NSGAGSGGSSGSDGASGS | 171 |
| | | RD | |
| LINK83 | Linker | GSLDG | 172 |
| LINK84 | Linker | SLDG | 173 |
| LINK85 | Linker | GSW | — |
| LINK86 | Linker | GSGGGSGGGSGS | 174 |
| LINK87 | Linker | H | — |
| LINK88 | Linker | GSGSGSGSGSG | 175 |
| LINK89 | Linker | GGGGSLVPRGSGGGGS | 176 |
| LINK90 | Linker | GGGGSLVPRGSGGGG | 177 |
| LINK91 | Linker | GGSGGHMGSGG | 178 |
| LINK92 | Linker | GGSGGSGGSGG | 179 |
| LINK93 | Linker | GGGSEGGGSEGGGSEGGG | 180 |
| LINK94 | Linker (GGSG)2 | GGSGGGSG | 181 |
| LINK95 | Linker | EVR | — |
| LINK96 | Linker | SWFE | 182 |
| LINK97 | Linker | SRFE | 183 |
| LINK98 | Linker | GWFE | 874 |

### Hinges

In some embodiments, the polypeptides of the disclosure may include one or more hinge regions. As used herein a hinge is a molecule containing one or more amino acids that connect two molecules or two parts of a molecule such as two regions of a polypeptide and facilitates confirmational or structural flexibility by allowing the each of the two molecules or parts to move relative to one another. In some embodiments, the hinge region may mediate the kinetics of lipid binding by providing the opening of the helices required to initiate lipid binding. In some embodiments, the hinge region sequence of the mutated to alter proteolytic stability of polypeptides of the disclosure. The hinge may be positioned within the structured sequence region, the N-terminal sequence region, the C-terminal sequence region, or between the N-terminal sequence region and the structured sequence region or the structured sequence region and the C-terminal sequence region or any other regions or fragments of the polypeptides of the disclosure. The hinge region can be any suitable sequence derived or obtained from any suitable molecule. The hinge sequence may be derived from all or part of an apolipoprotein. In some embodiments, the polypeptides of the disclosure may not include hinge regions. Table 6 provides non-limiting examples of hinge sequences.

TABLE 6
Hinge sequences

| | | | SEQ |
|---|---|---|---|
| | Description | | ID |
| Hinge ID | of Domain | Sequence | NO: |
| HINGE1 | APOE Hinge | PLVEQGRVRAATVGSLAGQPLQERAQAWGERLR | 184 |
| | | ARMEEMGSRTR | |
| HINGE2 | APOE Hinge | RAATVGSLAGQPLQER | 185 |
| HINGE3 | APOE Hinge | RAATVGSLAGQPLQERAQAWGERLRAR | 186 |
| HINGE4 | APOE Hinge | PLVEQGRVRAATVGSLAGQ | 187 |
| HINGE5 | APOE Hinge | PLQERAQAWGERLRARMEEMGSRTR | 188 |
| HINGE6 | APOE Hinge | PLVEQGRVRAA | 189 |
| HINGE7 | APOE Hinge | PLVEQGRVRADATVGSLAGQLG | 190 |
| HINGE8 | APOE Hinge | PLVEQGRVRADATVGSLAGQLGAQAWGERLRAR | 191 |
| | | LGPLMEEMGSRTR | |
| HINGE9 | APOE Hinge | PLVEQGRVRAAAVGSLAGQPLQERAQAWGERLR | 192 |
| | | ARMEEMGSRTR | |
| HINGE10 | B7-1 Hinge | KQEHFPDN | 193 |
| HINGE11 | C233P | VEPKSPDKTHTCPPCP | 194 |
| | Hinge | | |
| HINGE12 | C233S | LDPKSSDKTHTCPPCP | 195 |
| | Hinge | | |
| HINGE13 | CD28 Hinge | IEVMYPPPYLDNEKSNGTIIHVKGKHLCPSPLFPG | 196 |
| | | PSKP | |
| HINGE14 | CD8a Hinge | GGAVHTRGLDFA | 197 |
| HINGE15 | CD8a Hinge | TTTPAPRPPTPAPTIASQPLSLRPEACRPAAGGAV | 198 |
| | | HTRGLDFACD | |
| HINGE16 | CD8a Hinge | AKPTTTPAPRPPTPAPTIASQPLSLRPEACRPAAG | 199 |
| | | GAVHTRGLDFACD | |
| HINGE17 | CD8a Hinge | TTTPAPRPPTPAPTIASQPLSLRPEACRPAAGGAV | 200 |
| | | HTRGLDFACDEPKSPDKTHTCPPCPAPPVAGPSV | |
| | | FLFPPKPKDT | |
| HINGE18 | CD8a Hinge | LDFACD | 201 |
| HINGE19 | CD8a Hinge | PAKPTTTPAPRPPTPAPTIASQPLSLRPEACRPAA | 202 |
| | | GGAVHTRGLDFACDIY | |
| HINGE20 | CD8a Hinge | TTPAPRPPTPAPTIASQPLSLRPEACRPAAGGAVH | 203 |
| | | TRGLDFACD | |
| HINGE21 | CD8a Hinge | TTTPAPRPPTPAPTIASQPLSLRPEACRPAAGGAV | 204 |
| | | HTRGLDFACDIY | |
| HINGE22 | Delta5 | LDKTHTCPPCP | 205 |
| | Hinge | | |
| HINGE23 | FCgr2b | FSIPQANHSHSGDYHCTGNIGYTLYSSKPVTITVQ | 206 |
| | Hinge | AP | |
| HINGE24 | FCRIIα | GLAVSTISSFFPPGYQ | 207 |
| | Hinge | | |
| HINGE25 | Hinge | DKTHT | 208 |
| HINGE26 | Hinge | CPPC | 209 |
| HINGE27 | Hinge | CPEPKSCDTPPPCPR | 210 |
| HINGE28 | Hinge | ELKTPLGDTTHT | 211 |
| HINGE29 | Hinge | KSCDKTHTCP | 212 |
| HINGE30 | Hinge | KCCVDCP | 213 |
| HINGE31 | Hinge | KYGPPCP | 214 |
| HINGE32 | Hinge | YVTVSSQDPAEPKSPDKTHTCPPCPAPELLGGPS | 215 |
| | | VFLFPPKPKDTLMISRTPEVTCVVVDVSHEDPEV | |
| | | KFNWYVDGVEVHNAKTKPREEQYNSTYRVVSVLT | |
| | | VLHQDWLNGKEYKCKVSNKALPAPIEKTISKAKG | |
| | | QPREPQVYTLPPSRDELTKNQVSLTCLVKGFYPS | |
| | | DIAVEWESNGQPENNYKTTPPVLDSDGSFFLYSK | |
| | | LTVDKSRWQQGNVFSCSVMHEALHNHYTQKSLS | |
| | | LSPGKKDPK | |
| HINGE33 | Hinge | KPTTTPAPRPPTPAPTIASQPLSLRPEACRPAAGG | 216 |
| | | AVHTRGLDFA | |
| HINGE34 | Hinge | LEPKSCDKTHTCPPCP | 217 |
| HINGE35 | Hinge | KPTTTPAPRPPTPAPTIASQPLSLRPEACRPAAGG | 218 |
| | | AVHTRGLD | |
| HINGE36 | Hinge | ELKTPLGDTHTCPRCP | 219 |
| HINGE37 | Hinge | EPKSCDTPPPCPRCP | 220 |
| HINGE38 | Hinge | ESKYGPPCPSCP | 221 |
| HINGE39 | Hinge (IgG2) | ERKCCVECPPCP | 222 |
| HINGE40 | IgD Hinge | RWPESPKAQASSVPTAQPQAEGSLAKATTAPATT | 223 |
| | | RNTGRGGEEKKKEKEKEEQEERETKTPECPSHT | |
| | | QPLGVYLLTPAVQDLWLRDKATFTCFVVGSDLKD | |
| | | AHLTWEVAGKVPTGGVEEGLLERHSNGSQSQHS | |
| | | RLTLPRSLWNAGTSVTCTLNHPSLPPQRLMALRE | |
| | | PAAQAPVKLSLNLLASSDPPEAASWLLCEVSGFS | |
| | | PPNILLMWLEDQREVNTSGFAPARPPPQPGSTTF | |
| | | WAWSVLRVPAPPSPQPATYTCVVSHEDSRTLLN | |
| | | ASRSLEVSYVTDH | |
| HINGE41 | IgD Hinge | RWPESPKAQASSVPTAQPQAEGSLAKATTAPATT | 224 |
| | | RNTGRGGEEKKKEKEKEEQEERETKTPECPSHT | |
| | | QPLGVYLLTPAVQDLWLRDKATFTCFVVGSDLKD | |
| | | AHLTWEVAGKVPTGGVEEGLLERHSNGSQSQHS | |
| | | RLTLPRSLWNAGTSVTCTLHPSLPPQRLMALREP | |
| | | AAQAPVKLSLNLLASSDPPEAASWLLCEVSGFSP | |
| | | PNILLMWLEDQREVNTSGFAPARPPPQPGSTTFW | |
| | | AWSVLRVPAPPSPQPATYTCVVSHEDSRTLLNAS | |
| | | RSLEVSYVTDH | |
| HINGE42 | IgD Hinge | RWPESPKAQASSVPTAQPQAEGSLAKATTAPATT | 225 |
| | | RNTGRGGEEKKKEKEKEEQEERETKTPECPSHTQ | |
| | | PLGVYLLTPAVQDLWLRDKATFTCFVVGSDLKD | |
| | | AHLTWEVAGKVPTGGVEEGLLERHSNGSQSQHS | |
| | | RLTLPRSLWNAGTSVTCTLNHPSLPPQRLMALRE | |
| | | PAAQAPVKLSLNLLASSDPPEAASWLLCEVSGFS | |
| | | PPNILLMVVLEDQREVNTSGFAPARPPPQPGSTT | |
| | | FWAWSVLRVPAPPSPQPATYTCVVSHEDSRTLLN | |
| | | ASRSLEVSYVTDH | |
| HINGE43 | IgD Hinge | ESPKAQASSVPTAQPQAEGSLAKATTAPATTRNT | 226 |
| | | GRGGEEKKKEKEKEEQEERETKTP | |
| HINGE44 | IgG1 Hinge | AEPKSPDKTHTCPPCPAPPVAGPSVFLFPPKPKD | 227 |
| | | TLMIARTPEVTCVVVDVSHEDPEVKFNWYVDGVE | |
| | | VHNAKTKPREEQYNSTYRVVSVLTVLHQDWLNGK | |
| | | EYKCKVSNKALPAPIEKTISKAKGQPREPQVYTL | |
| | | PPSRDELTKNQVSLTCLVKGFYPSDIAVEWESNG | |
| | | QPENNYKTTPPVLDSDGSFFLYSKLTVDKSRWQQ | |
| | | GNVFSCSVMHEALHNHYTQKSLSLSPGKKD | |
| HINGE45 | IgG1 Hinge | EPKSCDKTHTCPPCP | 228 |
| HINGE46 | IgG1 Hinge | AEPKSPDKTHTCPPCPKDPK | 229 |
| HINGE47 | IgG1 Hinge | EPKSPDKTHTCPPCPAPPVAGPSVFLFPPKPKDT | 230 |
| | | LMIARTPEVTCVVVDVSHEDPEVKFNWYVDGVEV | |
| | | HNAKTKPREEQYNSTYRVVSVLTVLHQDWLNGKE | |
| | | VKCKVSNKALPAPIEKTISKAKGQPREPQVYTLP | |
| | | PSRDELTKNQVSLTCLVKGFYPSDIAVEWESNGQ | |
| | | PENNYKTTPPVLDSDGSFFLVSKLTVDKSRWQQG | |
| | | NVFSCSVMHEALHNHYTQKSLSLSPGKKD | |
| HINGE48 | IgG1 Hinge | SVFLFPPKPKDTL | 231 |
| HINGE49 | IgG1 Hinge | DPAEPKSPDKTHTCPPCPAPPVAGPSVFLFPPKP | 232 |
| | | KDTLMIARTPEVTCVVVDVSHEDPEVKFNWYVDG | |
| | | VEVHNAKTKPREEQYNSTYRVVSVLTVLHQDWLN | |
| | | GKEYKCKVSNKALPAPIEKTISKAKGQPREPQVYT | |
| | | LPPSRDELTKNQVSLTCLVKGFYPSDIAVEWESN | |
| | | GQPENNYKTTPPVLDSDGSFFLYSKLTVDKSRWQ | |
| | | QGNVFSCSVMHEALHNHYTQKSLSLSPGKK | |
| HINGE50 | IgG1 Hinge | EPKSPDKTHTCPPCPAPPVAGPSVFLFPPKPKDT | 233 |
| | | LMIARTPEVTCVVVDVSHEDPEVKFNWYVDGVEV | |
| | | HNAKTKPREEQYNSTYRVVSVLTVLHQDWLNGKE | |
| | | YKCKVSNKALPAPIEKTISKAKGQPREPQVYTLP | |
| | | PSRDELTKNQVSLTCLVKGFYPSDIAVEWESNGQ | |
| | | PENNYKTTPPVLDSDGSFFLYSKLTVDKSRWQQG | |
| | | NVFSCSVMHEALHNHYTQKSLSLSPGK | |
| HINGE51 | IgG1 Hinge | EPKSPDKTHTCPPCPAPPVAGPSVFLFPPKPKDT | 234 |
| | | LMIARTPEVTCVVVDVSHEDPEVKFNWYVDGVEV | |
| | | HNAKTKPREEQYNSTYRVVSVLTVLHQDWLNGKE | |
| | | YKCKVSNKALPAPIEKTISKAKGQPREPQVYTLP | |
| | | PSRDELTKNQVSLTCLVKGFYPSDIAVEWESNGQ | |
| | | PENNYKTTPPVLDSDGSFFLYSKLTVDKSRWQQG | |
| | | NVFSCSVMHEALHNHYTQKSLSLSPGKKDPK | |
| HINGE52 | IgG1 Hinge | VECPPCPAPPVAGPSVFLFPPKPKDTLMISRTPE | 235 |
| | | VTCVVVDVSHEDPEVKFNWYVDGVEVHNAKTKPR | |
| | | EEQYNSTYRVVSVLTVLHQDWLNGKEYKCKVSNK | |
| | | GLPSSIEKTISKAKGQPREPQVYTLPPSREEMTK | |
| | | NQVSLTCLVKGFYPSDIAVEWESNGQPENNYKTT | |
| | | PPVLDSDGSFFLYSKLTVDKSRWQQGNVFSCSV | |
| | | MHEALHNHYTQKSLSLSPGK | |
| HINGE53 | IgG3 Hinge | ELKTPLGDTTHTCPRCP | 236 |
| HINGE54 | IgG3 Hinge | ELKTPLGDTHTCPRCPEPKSCDTPPPCPRCPEPK | 237 |
| | | SCDTPPPCPRCPEPKSCDTPPPCPRCP | |
| HINGE55 | IgG4 Hinge | SPNMVPHAHHAQ | 238 |
| HINGE56 | IgG4 Hinge | GQPREPQVYTLPPSQEEMTKNQVSLTCLVKGFYP | 239 |
| | | SDIAVEWESNGQPENNYKTTPPVLDSDGSFFLYS | |
| | | RLTVDKSRWQEGNVFSCSVMHEALHNHYTQKSL | |
| | | SLSLGK | |
| HINGE57 | IgG4 Hinge | ESKYGPPCPPCPGGGSSGGGSGGQPREPQVYT | 240 |
| | | LPPSQEEMTKNQVSLTCLVKGFYPSDIAVEWESN | |
| | | GQPENNYKTTPPVLDSDGSFFLYSRLTVDKSRW | |
| | | QEGNVFSCSVMHEALHNHYTQKSLSLSLGK | |
| HINGE58 | IgG4 Hinge | ESKYGPPCPSCPAPEFEGGPSVFLFPPKPKDTLM | 241 |
| | | ISRTPEVTCVVVDVSQEDPEVQFNWYVDGVEVH | |
| | | QAKTKPREEQFNSTYRVVSVLTVLHQDWLNGKE | |
| | | YKCKVSNKGLPSSIEKTISKAKGQPREPQVYTLPP | |
| | | SQEEMTKNQVSLTCLVKGFVPSDIAVEWESNGQP | |
| | | ENNYKTTPPVLDSDGSFFLYSRLTVDKSRWQEGN | |
| | | VFSCSVMHEALHNHYTQKSLSLSLGK | |
| HINGE59 | IgG4 Hinge | ESKYGPPCPPCPAPEFEGGPSVFLFPPKPKDTLM | 242 |
| | | ISRTPEVTCVVVDVSQEDPEVQFNWYVDGVEVH | |
| | | QAKTKPREEQFNSTYRVVSVLTVLHQDWLNGKE | |
| | | YKCKVSNKGLPSSIEKTISKAKGQPREPQVYTLPP | |
| | | SQEEMTKNQVSLTCLVKGFVPSDIAVEWESNGQP | |
| | | ENNYKTTPPVLDSDGSFFLYSRLTVDKSRWQEGN | |
| | | VFSCSVMHEALHNHYTQKSLSLSLGK | |
| HINGE60 | IgG4 Hinge | ESKYGPPCPPCP | 243 |
| HINGE61 | IgG4 Hinge | YGPPCPPCP | 244 |
| HINGE62 | IgG4 Hinge | KYGPPCPPCP | 245 |
| HINGE63 | IgG4 Hinge | EVVKYGPPCPPCP | 246 |
| HINGE64 | IgG4 Hinge | ESKYGPPCPSCPAPEFLGGPSVFLFPPKPKDTLMI | 247 |
| | | SRTPEVTCVVVDVSQEDPEVQFNWYVDGVEVHN | |
| | | AKTKPREEQFNSTYRVVSVLTVLHQDWLNGKEYK | |
| | | CKVSNKGLPSSIEKTISKAKGQPREPQVYTLPPSQ | |
| | | EEMTKNQVSLTCLVKGFYPSDIAVEWESNGQPEN | |
| | | NYKTTPPVLDSDGSFFLYSRLTVDLSRWQEGNVF | |
| | | SCSVMHEALHNHYTQKSLSLSLGK | |
| HINGE65 | IgG4 Hinge | ESKYGPPCPPCPAPEFLGGPSVFLFPPKPKDTLMI | 248 |
| | | SRTPEVTCVVVDVSQEDPEVQFNWYVDGVEVHN | |
| | | AKTKPREEQFNSTYRVVSVLTVLHQDWLNGKEYK | |
| | | CKVSNKGLPSSIEKTISKAKGQPREPQVYTLPPSQ | |
| | | EEMTKNQVSLTCLVKGFYPSDIAVEWESNGQPEN | |
| | | NYKTTPPVLDSDGSFFLYSRLTVDKSRWQEGNVF | |
| | | SCSVMHEALHNHYTQKSLSLSLGK | |
| HINGE66 | IgG4 Hinge | ESKYGPPCPPCPGQPREPQVYTLPPSQEEMTKN | 249 |
| | | QVSLTCLVKGFYPSDIAVEWESNGQPENNYKTTP | |
| | | PVLDSDGSFFLYSRLTVDKSRWQEGNVFSCSVM | |
| | | HEALHNHYTQKSLSLSLGK | |
| HINGE67 | IgG1 Hinge | EPKSPDKTHTCPPCPAPPVAGPSVFLFPPKPKDT | 250 |
| | | LMIARTPEVTCVVVDVSHEDPEVKFNWYVDGVEV | |
| | | HNAKTKPREEQYNSTYRVVSVLTVLHQDWLNGK | |
| | | EYKCKVSNKALPAPIEKTISKAKGQPREPQVYTLP | |
| | | PSRDELTKNQVSLTCLVKGFYPSDIAVEWESNGQ | |
| | | PENNYKTIPPVLDSDGSFFLYSKLTVDKSRWQQG | |
| | | NVFSCSVMHEALHNHYTQKSLSLSPGK | |
| HINGE68 | IgG1 Hinge | EPKSPDKTHTCPPCPAPPVAGPSVFLFPPKPKDT | 251 |
| | | LMIARTPEVTCVVVDVSHEDPEVKFNWYVDGVEV | |
| | | HNAKTKPREEQYNSTYRVVSVLTVLHQDWLNGK | |
| | | EYKCKVSNKALPAPIEKTISKAKGQPREPQVYTLP | |
| | | PSRDELTKNQVSLTCLVKGFYPSDIAVEWESNGQ | |
| | | PENNYKTIPPVLDSDGSFFLYSKLTVDKSRWQQG | |
| | | NVFSCSVMHEALHNHYTQKSLSLSPGK | |
| Hinge69 | Hinge | PLVEQGRQRTANLGAGAAQPLRDRAQALSDRIR | 875 |
| | | GRLEEVGNQAR | |

### Signal Sequence

Polypeptides of the disclosure may further include one or more additional features such as one or more signal sequences.

Signal sequences (sometimes referred to as signal peptides, targeting signals, target peptides, localization sequences, transit peptides, leader sequences or leader peptides) direct proteins or polypeptides to their designated cellular and/or extracellular locations. Protein signal sequences play a central role in the targeting and translocation of nearly all secreted proteins and many integral membrane proteins. In some embodiments, the signal sequences may direct the polypeptides of the disclosure for secretion.

A signal sequence is a short (5-30 amino acids long) peptide present at the N-terminus of the majority of newly synthesized proteins that are destined towards a particular location. Signal sequences can be recognized by signal recognition particles (SRPs) and cleaved using type I and type II signal peptide peptidases. In some embodiments, a signal sequence may be, although not necessarily, located at the N-terminus or C-terminus of the polypeptide, and may be, although not necessarily, be cleaved off the polypeptides of the disclosure.

In some embodiments, a signal peptide according to the disclosure is situated at the N-terminal end of the protein of interest or at the N-terminal end of the pro-protein form of the protein of interest. A signal peptide according to the disclosure may be of eukaryotic origin e.g., the signal peptide of a eukaryotic protein, e.g., of mammalian origin, e.g., the signal peptide of a mammalian protein, more preferably of human origin, e.g., the signal peptide of a mammalian protein.

In some embodiments, the signal sequence may be derived from an apolipoprotein. Non-limiting examples of apolipoprotein signal sequences are provided in Table 7. All signal sequence parent apolipoprotein proteins in Table 7 are derived from human apolipoprotein.

TABLE 7
Signal sequence

| Signal | | | SEQ |
|---|---|---|---|
| sequence | Signal sequence | | ID |
| ID | parent protein | Signal Sequence (AA sequence) | NO: |
| SS1 | Apolipoprotein E | MKVLWAALLVTFLAGCQA | 252 |
| SS2 | Apolipoprotein A1 | MKAAVLTLAVLFLTGSQA | 253 |
| SS3 | Apolipoprotein A2 | MKLLAATVLLLTICSLEG | 254 |
| SS4 | Apolipoprotein A4 | MFLKAVVLTLALVAVAGARA | 255 |
| SS5 | Apolipoprotein B | MDPPRPALLALLALPALLLLLLAGARA | 256 |
| SS6 | Apolipoprotein C2 | MGTRLLPALFLVLLVLGFEVQG | 257 |
| SS7 | Apolipoprotein C3 | MQPRVLLVVALLALLASARA | 258 |
| SS8 | Apolipoprotein C1 | MRLFLSLPVLVVVLSIVLEGPAPAQG | 259 |
| SS9 | Apolipoprotein D | MVMLLLLLSALAGLFGAAEG | 260 |
| SS10 | Apolipoprotein M | MFHQIWAALLYFYGIILNSIYQ | 261 |
| SS11 | Apolipoprotein(a) | MEHKEVVLLLLLFLKSAAP | 262 |
| SS12 | lgK (Murine) | METDTLLLWVLLLWVPGSTGD | 263 |
| SS13 | lgK (Murine) | METDTLLLWVLLLWVPGSTG | 264 |
| SS14 | Apolipoprotein E | MKVLWAALLVTFLAGCQAMKVLWAAL | 265 |
| | | LVTFLAGCQA | |
| SS15 | Apolipoprotein A | MKAAVLTLAVLFLTGSQARHFWQQ | 266 |

In addition to signal sequences naturally occurring such as from a secreted protein, a signal sequence may be a variant modified from a known signal sequence of a protein. For example, U.S. Pat. NOs.: 8,258,102 and 9,133,265 to Sleep disclose a modified albumin signal sequence having a secretion signal and an additional X1-X2-X3-X4-X5-motif which can increase protein secretion; U.S. Pat. No. 9,279,007 to Do discloses signal sequences of modified fragments of human immunoglobulin heavy chain binding protein (Bip) that can enhance protein expression and secretion; U.S. Pat. No. 8,148,494 to Leonhartsberger et al., discloses a signal peptide with a cleavage site that can be fused with a recombinant protein; the contents of each of which are incorporated by reference in their entirety.

Signal sequences may be selected based on their compatibility with the secretory pathway of the cell type of interest so that the payloads is presented on the surface of the T cell. Other signal sequence variants may be used in the present polypeptide may include those discussed in U.S. patent application publication No.: 2007/0141666; POT patent application publication No.: 1993/018181; the contents of each of which are incorporated herein by reference in their entirety.

In other embodiments, a signal sequence may be a heterogeneous signal sequence from other organisms such as virus, yeast and bacteria, which can direct a polypeptide to a particular cellular site, such as a nucleus (e.g., EP 1209450). Other examples may include Aspartic Protease (NSP24) signal sequences from Trichoderma that can increase secretion of fused protein such as enzymes (e.g., U.S. Pat. No. 8,093,016 to Cervin and Kim), bacterial lipoprotein signal sequences (e.g., PCT application publication NO.: 1991/09952 to Lau and Rioux), E. coli enterotoxin II signal peptides (e.g., U.S. Pat. No. 6,605,697 to Kwon et al.), E. coli secretion signal sequence (e.g., U.S. patent publication NO.: 2016/090404 to Malley et al.), a lipase signal sequence from a methylotrophic yeast (e.g., U.S. Pat. No. 8,975,041), and signal peptides for DNases derived from Coryneform bacteria (e.g., U.S. Pat. No. 4,965,197); the contents of each of which are incorporated herein by reference in their entirety.

### Fusion Polypeptides

In some embodiments, polypeptides of the disclosure may be fusion polypeptides. As used herein, the term “fusion polypeptide” may refer to a single, contiguous peptide molecule containing two or more parent proteins or regions or portions thereof that are typically encoded by different genes. Fusion polypeptides may have functional properties derived from each of the original proteins.

The components of the fusion polypeptides may be arranged from amino terminal to carboxy terminal of successive regions of the fusion protein. The regions may be directly linked to each other or linked via linkers. Protein linkers aid fusion protein design by providing appropriate spacing between domains, supporting correct folding of the fusion polypeptide. In some embodiments, the fusion polypeptide may include one or more parent proteins such as, but not limited to, APOE, APOA, APOB, APOC, APOD, APOF, APOH, APOJ, APOL, APOM, APOO or a variant thereof.

In some embodiments, fusion polypeptides of the disclosure may include a structured sequence region from a first apolipoprotein fused to a structured sequence region of a second apolipoprotein, where the first apolipoprotein may be APOE or APOA and the second apolipoprotein may be APOE or APOA.

In some embodiments, the fusion polypeptides of the disclosure may include a structured sequence region, an N-terminal sequence region, a C-terminal sequence region, each independently derived from the same or two or more apolipoproteins, such as but not limited to, APOE, APOA, APOB, APOC, APOD, APOF, APOH, APOJ, APOL, APOM, APOO or a variant thereof.

In some embodiments, the fusion polypeptide may include one or more of a region, a motif, a surface manifestation, a local confirmational shape, a fold, a loop, a half-loop, a domain, a half domain, a site, a terminus or any combination thereof from a parent protein such as, but not limited to, APOE, APOA, APOB, APOC, APOD, APOF, APOH, APOJ, APOL, APOM, APOO, or a variant thereof.

### Polypeptide Sequences

In some embodiments, the apolipoprotein may include two or more polypeptide feature sequences disclosed herein. In some embodiments, the apolipoprotein sequence is a sequence disclosed in Table 8.

TABLE 8
Apolipoprotein

| PP ID | Description | SEQ ID NO |
|---|---|---|
| PP1 | NTERM1; SSRP1; SSRP2; SSRP3; SSRP4; SSRP5; SSRP6; | 267 |
| | SSRP7; SSRP8; SSRP22; SSRP10; SSRP11; HINGE1; SSRP12; | |
| | LINK95; SSRP13; SSRP14; LINK96; SSRP15; SSRP16; CTERM1 | |
| PP2 | NTERM1; SSRP1; SSRP2; SSRP3; SSRP4; SSRP5; SSRP6; | 268 |
| | SSRP7; SSRP8; SSRP9; SSRP10; SSRP11; HINGE1; SSRP12; | |
| | LINK95; SSRP13; SSRP14; LINK96; SSRP15; SSRP16; CTERM1 | |
| PP3 | NTERM1; SSRP1; SSRP2; SSRP3; SSRP4; SSRP21; SSRP6; | 269 |
| | SSRP7; SSRP8; SSRP9; SSRP10; SSRP11; HINGE1; SSRP12; | |
| | LINK95; SSRP13; SSRP14; LINK96; SSRP15; SSRP16; CTERM1 | |
| PP4 | NTERM1; SSRP1; SSRP2; SSRP3; SSRP4; SSRP5; SSRP6; | 270 |
| | SSRP7; SSRP8; SSRP9; SSRP10; SSRP11; HINGE1; SSRP12; | |
| | LINK95; SSRP13; SSRP78; LINK97; SSRP18; SSRP19; CTERM1 | |
| PP5 | NTERM1; SSRP1; SSRP2; SSRP3; SSRP4; SSRP5; SSRP6; | 271 |
| | SSRP17; SSRP8; SSRP9; SSRP10; SSRP11; HINGE1; SSRP12; | |
| | LINK95; SSRP13; SSRP14; LINK96; SSRP15; SSRP16; CTERM1 | |
| PP6 | SSRP1; SSRP2; SSRP3; SSRP4; SSRP5; SSRP6; SSRP7; | 272 |
| | SSRP8; SSRP9; SSRP10; SSRP11; HINGE1; SSRP12; LINK95; | |
| | SSRP13; SSRP14; LINK96; SSRP15; SSRP16 | |
| PP7 | SSRP1; SSRP2; SSRP3; SSRP4; SSRP5; SSRP6; SSRP7; | 273 |
| | SSRP8; SSRP9; SSRP10; SSRP11; HINGE1; SSRP12; LINK95; | |
| | SSRP13; SSRP14; LINK96; SSRP15; SSRP16; CTERM1 | |
| PP8 | NTERM1; SSRP1; SSRP2; SSRP3; SSRP4; SSRP5; SSRP6; | 274 |
| | SSRP7; SSRP8; SSRP9; SSRP10; SSRP11; HINGE1; SSRP12; | |
| | LINK95; SSRP13; SSRP14; LINK96; SSRP15; SSRP16 | |
| PP9 | NTERM1; SSRP1; SSRP2; SSRP3; SSRP4; SSRP5; SSRP6; | 275 |
| | SSRP7; SSRP8; SSRP9; SSRP10; SSRP11; HINGE1; SSRP12; | |
| | SSRP13; SSRP14; SSRP15; SSRP16; CTERM1 | |
| PP10 | NTERM1; SSRP1; SSRP2; SSRP3; SSRP4; SSRP5; SSRP6; | 276 |
| | SSRP7; SSRP8; SSRP9; SSRP10; SSRP11; SSRP12; LINK95; | |
| | SSRP13; SSRP14; LINK96; SSRP15; SSRP16; CTERM1 | |
| PP11 | NTERM1; SSRP1; SSRP2; SSRP3; SSRP4; SSRP5; SSRP6; | 277 |
| | SSRP7; SSRP8; SSRP9; SSRP10; SSRP11; SSRP12; SSRP13; | |
| | SSRP14; SSRP15; SSRP16; CTERM1 | |
| PP12 | NTERM1; SSRP1; SSRP2; SSRP3; SSRP4; SSRP5; SSRP6; | 278 |
| | SSRP17; SSRP8; SSRP9; SSRP10; SSRP11; HINGE1; SSRP12; | |
| | SSRP13; SSRP14; SSRP15; SSRP16; CTERM1 | |
| PP13 | NTERM1; SSRP1; SSRP2; SSRP3; SSRP4; SSRP21; SSRP6; | 279 |
| | SSRP7; SSRP8; SSRP22; SSRP10; SSRP11; HINGE1; SSRP12; | |
| | SSRP13; SSRP14; SSRP15; SSRP16; CTERM1 | |
| PP14 | NTERM1; SSRP1; SSRP2; SSRP3; SSRP4; SSRP5; SSRP6; | 280 |
| | SSRP7; SSRP8; SSRP9; SSRP10; SSRP11; HINGE1; SSRP12; | |
| | SSRP13; SSRP14; SSRP15; SSRP16 | |
| PP15 | NTERM1; SSRP1; SSRP2; SSRP3; SSRP4; SSRP5; SSRP6; | 281 |
| | SSRP7; SSRP8; SSRP9; SSRP10; SSRP11; HINGE1; SSRP12; | |
| | SSRP13; SSRP78; LINK97; SSRP18; SSRP19; CTERM1 | |
| PP16 | NTERM1; SSRP1; SSRP2; SSRP3; SSRP4; SSRP5; SSRP6; | 282 |
| | SSRP7; SSRP8; SSRP9; SSRP10; SSRP11; HINGE1; SSRP12; | |
| | SSRP13; SSRP78; SSRP18; SSRP19 | |
| PP17 | NTERM1; SSRP1; SSRP2; SSRP3; SSRP4; SSRP5; SSRP6; | 283 |
| | SSRP17; SSRP8; SSRP9; SSRP10; SSRP11; HINGE1; SSRP12; | |
| | SSRP13; SSRP78; SSRP18; SSRP19; CTERM1 | |
| PP18 | NTERM1; SSRP1; SSRP2; SSRP3; SSRP4; SSRP21; SSRP6; | 284 |
| | SSRP7; SSRP8; SSRP22; SSRP10; SSRP11; HINGE1; SSRP12; | |
| | SSRP13; SSRP78; SSRP18; SSRP19; CTERM1 | |
| PP19 | NTERM1; SSRP1; SSRP2; SSRP3; SSRP4; SSRP5; SSRP6; | 285 |
| | SSRP7; SSRP8; SSRP9; SSRP10; SSRP11; HINGE1; SSRP12; | |
| | SSRP13; SSRP78; SSRP15; SSRP16; CTERM1 | |
| PP20 | NTERM1; SSRP1; SSRP2; SSRP3; SSRP4; SSRP5; SSRP6; | 286 |
| | SSRP7; SSRP8; SSRP9; SSRP10; SSRP11; HINGE1; SSRP12; | |
| | SSRP13; SSRP14; SSRP18; SSRP16; CTERM1 | |
| PP21 | NTERM1; SSRP1; SSRP2; SSRP3; SSRP4; SSRP5; SSRP6; | 287 |
| | SSRP7; SSRP8; SSRP9; SSRP10; SSRP11; HINGE1; SSRP12; | |
| | SSRP13; SSRP14; SSRP15; SSRP20; CTERM1 | |
| PP22 | NTERM1; SSRP1; SSRP2; SSRP3; SSRP4; SSRP5; SSRP6; | 288 |
| | SSRP7; SSRP8; SSRP9; SSRP10; SSRP11; HINGE1; SSRP12; | |
| | SSRP13; SSRP14; SSRP15; SSRP79; CTERM1 | |
| PP23 | SSRP32; SSRP1; SSRP2; SSRP3; SSRP4; SSRP5; SSRP6; | 289 |
| | SSRP7; SSRP8; SSRP9; SSRP10; SSRP11; SSRP12; SSRP13; | |
| | SSRP14; SSRP15; SSRP16; CTERM1 | |
| PP24 | SSRP32; SSRP33; SSRP1; SSRP2; SSRP3; SSRP4; SSRP5; | 290 |
| | SSRP6; SSRP7; SSRP8; SSRP9; SSRP10; SSRP11; SSRP12; | |
| | SSRP13; SSRP14; SSRP15; SSRP16; CTERM1 | |
| PP25 | SSRP32; SSRP33; SSRP34; SSRP1; SSRP2; SSRP3; SSRP4; | 291 |
| | SSRP5; SSRP6; SSRP7; SSRP8; SSRP9; SSRP10; SSRP11; | |
| | SSRP12; SSRP13; SSRP14; SSRP15; SSRP16; CTERM1 | |
| PP26 | NTERM1; SSRP1; SSRP2; SSRP3; SSRP4; SSRP21; SSRP6; | 292 |
| | SSRP17; SSRP8; SSRP22; SSRP10; SSRP11; HINGE1; SSRP12; | |
| | SSRP13; SSRP14; SSRP15; SSRP16; CTERM1 | |
| PP27 | SSRP32; SSRP33; SSRP34; SSRP1; SSRP2; SSRP3; SSRP4; | 293 |
| | SSRP5; SSRP6; SSRP7; SSRP8; SSRP9; SSRP10; SSRP11; | |
| | SSRP12; SSRP13; SSRP14; SSRP15; SSRP16 | |
| PP28 | NTERM1; SSRP1; SSRP2; SSRP3; SSRP4; SSRP5; SSRP6; | 294 |
| | SSRP7; SSRP8; SSRP9; SSRP10; SSRP11; HINGE1; SSRP80; | |
| | LINK95; SSRP13; SSRP14; LINK96; SSRP15; SSRP16; CTERM1 | |
| PP29 | SSRP32; SSRP33; SSRP34; SSRP35; SSRP36; SSRP37; | 295 |
| | SSRP38; SSRP39; SSRP40; SSRP41; SSRP42; SSRP43; | |
| | SSRP44; SSRP45; SSRP46; SSRP47; SSRP48; CTERM2 | |
| PP30 | NTERM2; SSRP31; SSRP32; SSRP33; SSRP34; SSRP35; | 296 |
| | SSRP36; SSRP37; SSRP38; SSRP39; SSRP40; SSRP41; | |
| | SSRP42; SSRP43; SSRP44; SSRP45; SSRP46; SSRP47; | |
| | SSRP48; CTERM2 | |
| PP31 | NTERM1; SSRP1; SSRP2; SSRP3; SSRP4; SSRP5; SSRP6; | 297 |
| | SSRP17; SSRP8; SSRP9; SSRP10; SSRP11; HINGE1; SSRP12; | |
| | SSRP13; SSRP78; SSRP15; SSRP16; CTERM1 | |
| PP32 | NTERM1; SSRP1; SSRP2; SSRP3; SSRP4; SSRP5; SSRP6; | 298 |
| | SSRP7; SSRP8; SSRP9; SSRP10; SSRP11; HINGE1; SSRP80; | |
| | SSRP13; SSRP78; SSRP15; SSRP16; CTERM1 | |
| PP33 | NTERM1; SSRP1; SSRP2; SSRP3; SSRP4; SSRP5; SSRP6; | 299 |
| | SSRP17; SSRP8; SSRP9; SSRP10; SSRP11; HINGE1; SSRP80; | |
| | SSRP13; SSRP78; SSRP15; SSRP16; CTERM1 | |
| PP34 | NTERM1; SSRP1; SSRP2; SSRP3; SSRP4; SSRP5; SSRP6; | 300 |
| | SSRP17; SSRP8; SSRP9; SSRP10; SSRP11; SSRP12; SSRP13; | |
| | SSRP78; SSRP15; SSRP16; CTERM1 | |
| PP35 | NTERM1; SSRP1; SSRP2; SSRP3; SSRP4; SSRP5; SSRP6; | 301 |
| | SSRP7; SSRP8; SSRP9; SSRP10; SSRP11; SSRP80; SSRP13; | |
| | SSRP78; SSRP15; SSRP16; CTERM1 | |
| PP36 | NTERM1; SSRP1; SSRP2; SSRP3; SSRP4; SSRP5; SSRP6; | 302 |
| | SSRP17; SSRP8; SSRP9; SSRP10; SSRP11; SSRP80; SSRP13; | |
| | SSRP78; SSRP15; SSRP16; CTERM1 | |
| PP37 | NTERM1; SSRP1; SSRP2; SSRP3; SSRP4; SSRP5; SSRP6; | 303 |
| | SSRP7; SSRP8; SSRP9; SSRP10; SSRP11; HINGE6; SSRP12; | |
| | SSRP13; SSRP14; SSRP15; SSRP16; CTERM1 | |
| PP38 | NTERM1; SSRP1; SSRP2; SSRP3; SSRP4; SSRP5; SSRP6; | 304 |
| | SSRP7; SSRP8; SSRP9; SSRP10; SSRP11; HINGE7; SSRP12; | |
| | SSRP13; SSRP14; SSRP15; SSRP16; CTERM1 | |
| PP39 | NTERM1; SSRP1; SSRP2; SSRP3; SSRP4; SSRP5; SSRP6; | 305 |
| | SSRP7; SSRP8; SSRP9; SSRP10; SSRP11; HINGE8; SSRP12; | |
| | SSRP13; SSRP14; SSRP15; SSRP16; CTERM1 | |
| PP40 | NTERM2; SSRP31; SSRP32; SSRP33; SSRP34; SSRP5; SSRP6; | 306 |
| | SSRP7; SSRP8; SSRP9; SSRP10; SSRP41; SSRP42; SSRP43; | |
| | SSRP44; SSRP45; SSRP46; SSRP47; SSRP48; CTERM2 | |
| PP41 | NTERM1; SSRP1; SSRP2; SSRP3; SSRP4; SSRP35; SSRP36; | 307 |
| | SSRP37; SSRP38; SSRP39; SSRP40; SSRP11; HINGE1; SSRP12; | |
| | LINK95; SSRP13; SSRP14; LINK96; SSRP15; SSRP16; CTERM1 | |
| PP42 | NTERM1; SSRP1; SSRP2; SSRP3; SSRP4; SSRP35; SSRP36; | 308 |
| | SSRP37; SSRP38; SSRP39; SSRP40; SSRP11; SSRP12; | |
| | SSRP13; SSRP14; SSRP15; SSRP16; CTERM1 | |
| PP43 | SSRP32; SSRP33; SSRP34; SSRP35; SSRP5; SSRP6; SSRP7; | 309 |
| | SSRP8; SSRP9; SSRP10; SSRP42; SSRP43; SSRP44; SSRP45; | |
| | SSRP46; SSRP47; SSRP48; CTERM2 | |
| PP44 | SSRP1; SSRP2; SSRP3; SSRP4; SSRP5; SSRP6; SSRP7; | 310 |
| | SSRP8; SSRP9; SSRP10; SSRP11; HINGE1; SSRP12; SSRP13; | |
| | SSRP14; SSRP15; SSRP16 | |
| PP45 | SSRP1; SSRP2; SSRP3; SSRP4; SSRP5; SSRP6; SSRP7; | 311 |
| | SSRP8; SSRP9; SSRP10; SSRP11; SSRP12; SSRP13; SSRP14; | |
| | SSRP15; SSRP16 | |
| PP46 | SSRP1; SSRP2; SSRP3; SSRP4; SSRP5; SSRP6; SSRP7; | 312 |
| | SSRP8; SSRP9; SSRP10; SSRP11; LINK12; SSRP12; SSRP13; | |
| | SSRP14; SSRP15; SSRP16 | |
| PP47 | SSRP1; SSRP2; SSRP3; SSRP4; SSRP5; SSRP6; SSRP7; | 313 |
| | SSRP8; SSRP9; SSRP10; SSRP11; LINK7; SSRP12; SSRP13; | |
| | SSRP14; SSRP15; SSRP16 | |
| PP48 | SSRP1; SSRP2; SSRP3; SSRP4; SSRP5; SSRP6; SSRP7; | 314 |
| | SSRP8; SSRP9; SSRP10; SSRP11; LINK8; SSRP12; SSRP13; | |
| | SSRP14; SSRP15; SSRP16 | |
| PP49 | SSRP1; SSRP2; SSRP3; SSRP4; SSRP5; SSRP6; SSRP7; | 315 |
| | SSRP8; SSRP9; SSRP10; SSRP11; LINK9; SSRP12; SSRP13; | |
| | SSRP14; SSRP15; SSRP16 | |
| PP50 | SSRP1; SSRP2; SSRP3; SSRP4; SSRP5; SSRP6; SSRP7; | 316 |
| | SSRP8; SSRP22; SSRP10; SSRP11; LINK12; SSRP12; SSRP13; | |
| | SSRP14; SSRP15; SSRP16 | |
| PP51 | SSRP1; SSRP2; SSRP3; SSRP4; SSRP5; SSRP6; SSRP7; | 317 |
| | SSRP8; SSRP22; SSRP10; SSRP11; LINK7; SSRP12; SSRP13; | |
| | SSRP14; SSRP15; SSRP16 | |
| PP52 | SSRP1; SSRP2; SSRP3; SSRP4; SSRP5; SSRP6; SSRP7; | 318 |
| | SSRP8; SSRP22; SSRP10; SSRP11; LINK8; SSRP12; SSRP13; | |
| | SSRP14; SSRP15; SSRP16 | |
| PP53 | SSRP1; SSRP2; SSRP3; SSRP4; SSRP5; SSRP6; SSRP7; | 319 |
| | SSRP8; SSRP22; SSRP10; SSRP11; LINK9; SSRP12; SSRP13; | |
| | SSRP14; SSRP15; SSRP16 | |
| PP54 | SSRP1; SSRP2; SSRP3; SSRP4; SSRP5; SSRP6; SSRP17; | 320 |
| | SSRP8; SSRP9; SSRP10; SSRP11; LINK12; SSRP12; SSRP13; | |
| | SSRP14; SSRP15; SSRP16 | |
| PP55 | SSRP1; SSRP2; SSRP3; SSRP4; SSRP5; SSRP6; SSRP17; | 321 |
| | SSRP8; SSRP9; SSRP10; SSRP11; LINK7; SSRP12; SSRP13; | |
| | SSRP14; SSRP15; SSRP16 | |
| PP56 | SSRP1; SSRP2; SSRP3; SSRP4; SSRP5; SSRP6; SSRP17; | 322 |
| | SSRP8; SSRP9; SSRP10; SSRP11; LINK8; SSRP12; SSRP13; | |
| | SSRP14; SSRP15; SSRP16 | |
| PP57 | SSRP1; SSRP2; SSRP3; SSRP4; SSRP5; SSRP6; SSRP17; | 323 |
| | SSRP8; SSRP9; SSRP10; SSRP11; LINK9; SSRP12; SSRP13; | |
| | SSRP14; SSRP15; SSRP16 | |
| PP58 | SSRP1; SSRP2; SSRP3; SSRP4; SSRP5; SSRP6; SSRP17; | 324 |
| | SSRP8; SSRP9; SSRP10; SSRP11; LINK12; SSRP12; SSRP13; | |
| | SSRP78; SSRP18; SSRP19 | |
| PP59 | SSRP1; SSRP2; SSRP3; SSRP4; SSRP5; SSRP6; SSRP17; | 325 |
| | SSRP8; SSRP9; SSRP10; SSRP11; LINK7; SSRP12; SSRP13; | |
| | SSRP78; SSRP18; SSRP19 | |
| PP60 | SSRP1; SSRP2; SSRP3; SSRP4; SSRP5; SSRP6; SSRP17; | 326 |
| | SSRP8; SSRP9; SSRP10; SSRP11; LINK8; SSRP12; SSRP13; | |
| | SSRP78; SSRP18; SSRP19 | |
| PP61 | SSRP1; SSRP2; SSRP3; SSRP4; SSRP5; SSRP6; SSRP17; | 327 |
| | SSRP8; SSRP9; SSRP10; SSRP11; LINK9; SSRP12; SSRP13; | |
| | SSRP78; SSRP18; SSRP19 | |
| PP62 | NTERM1; SSRP1; SSRP2; SSRP3; SSRP4; SSRP21; SSRP6; | 328 |
| | SSRP7; SSRP8; SSRP22; SSRP10; SSRP11; HINGE1; SSRP12; | |
| | LINK95; SSRP13; SSRP14; LINK96; SSRP15; SSRP16; CTERM1 | |
| PP63 | SS1; NTERM1; SSRP1; SSRP2; SSRP3; SSRP4; SSRP5; SSRP6; | 329 |
| | SSRP7; SSRP8; SSRP9; SSRP10; SSRP11; HINGE1; SSRP12; | |
| | LINK95; SSRP13; SSRP14; LINK96; SSRP15; SSRP16; CTERM1 | |
| PP64 | SS2; NTERM1; SSRP1; SSRP2; SSRP3; SSRP4; SSRP5; SSRP6; | 2 |
| | SSRP7; SSRP8; SSRP22; SSRP10; SSRP11; HINGE1; SSRP12; | |
| | LINK95; SSRP13; SSRP14; LINK96; SSRP15; SSRP16; CTERM1 | |
| PP65 | SS2; NTERM1; SSRP1; SSRP2; SSRP3; SSRP4; SSRP5; SSRP6; | 1 |
| | SSRP7; SSRP8; SSRP9; SSRP10; SSRP11; HINGE1; SSRP12; | |
| | LINK95; SSRP13; SSRP14; LINK96; SSRP15; SSRP16; CTERM1 | |
| PP66 | SS2; NTERM1; SSRP1; SSRP2; SSRP3; SSRP4; SSRP21; | 3 |
| | SSRP6; SSRP7; SSRP8; SSRP9; SSRP10; SSRP11; HINGE1; | |
| | SSRP12; LINK95; SSRP13; SSRP14; LINK96; SSRP15; SSRP16; | |
| | CTERM1 | |
| PP67 | SS2; NTERM1; SSRP1; SSRP2; SSRP3; SSRP4; SSRP5; SSRP6; | 330 |
| | SSRP7; SSRP8; SSRP9; SSRP10; SSRP11; HINGE1; SSRP12; | |
| | LINK95; SSRP13; SSRP78; LINK97; SSRP18; SSRP19; CTERM1 | |
| PP68 | SS2; NTERM1; SSRP1; SSRP2; SSRP3; SSRP4; SSRP5; SSRP6; | 331 |
| | SSRP17; SSRP8; SSRP9; SSRP10; SSRP11; HINGE1; SSRP12; | |
| | LINK95; SSRP13; SSRP14; LINK96; SSRP15; SSRP16; CTERM1 | |
| PP69 | SS14; SSRP1; SSRP2; SSRP3; SSRP4; SSRP5; SSRP6; SSRP7; | 332 |
| | SSRP8; SSRP9; SSRP10; SSRP11; HINGE1; SSRP12; LINK95; | |
| | SSRP13; SSRP14; LINK96; SSRP15; SSRP16 | |
| PP70 | SS14; SSRP1; SSRP2; SSRP3; SSRP4; SSRP5; SSRP6; SSRP7; | 333 |
| | SSRP8; SSRP9; SSRP10; SSRP11; HINGE9; SSRP12; LINK95; | |
| | SSRP13; SSRP14; LINK96; SSRP15; SSRP16 | |
| PP71 | SS14; NTERM1; SSRP1; SSRP2; SSRP3; SSRP4; SSRP5; | 334 |
| | SSRP6; SSRP7; SSRP8; SSRP9; SSRP10; SSRP11; HINGE9; | |
| | SSRP12; LINK95; SSRP13; SSRP14; LINK96; SSRP15; SSRP16; | |
| | CTERM1 | |
| PP72 | SS14; SSRP1; SSRP2; SSRP3; SSRP4; SSRP5; SSRP6; SSRP7; | 335 |
| | SSRP8; SSRP9; SSRP10; SSRP11; HINGE1; SSRP12; LINK95; | |
| | SSRP13; SSRP14; LINK96; SSRP15; SSRP16; CTERM1 | |
| PP73 | SS14; NTERM1; SSRP1; SSRP2; SSRP3; SSRP4; SSRP5; | 336 |
| | SSRP6; SSRP7; SSRP8; SSRP9; SSRP10; SSRP11; HINGE1; | |
| | SSRP12; LINK95; SSRP13; SSRP14; LINK96; SSRP15; SSRP16 | |
| PP74 | SS14; SSRP1; SSRP2; SSRP3; SSRP4; SSRP5; SSRP6; | 337 |
| | SSRP17; SSRP8; SSRP9; SSRP10; SSRP11; HINGE1; SSRP12; | |
| | LINK95; SSRP13; SSRP14; LINK96; SSRP15; SSRP16 | |
| PP75 | SS14; SSRP1; SSRP2; SSRP3; SSRP4; SSRP5; SSRP6; | 338 |
| | SSRP17; SSRP8; SSRP9; SSRP10; SSRP11; HINGE9; SSRP12; | |
| | LINK95; SSRP13; SSRP14; LINK96; SSRP15; SSRP16 | |
| PP76 | SS2; NTERM1; SSRP1; SSRP2; SSRP3; SSRP4; SSRP5; SSRP6; | 339 |
| | SSRP17; SSRP8; SSRP9; SSRP10; SSRP11; HINGE9; SSRP12; | |
| | LINK95; SSRP13; SSRP14; LINK96; SSRP15; SSRP16; CTERM1 | |
| PP77 | SS1; NTERM7; SSRP88; SSRP89; SSRP90; SSRP91; SSRP92; | 340 |
| | SSRP93; SSRP94; SSRP95; SSRP9; SSRP96; SSRP97; HINGE69; | |
| | SSRP98; LINK95; SSRP99; SSRP100; SSRP15; SSRP101; CTERM3 | |
| PP78 | SS2; NTERM3; SSRP1; SSRP2; SSRP3; SSRP4; SSRP5; SSRP6; | 341 |
| | SSRP7; SSRP8; SSRP9; SSRP10; SSRP11; HINGE1; SSRP12; | |
| | LINK95; SSRP13; SSRP14; LINK96; SSRP15; SSRP16; CTERM1 | |
| PP79 | SS2; NTERM4; SSRP1; SSRP2; SSRP3; SSRP4; SSRP5; SSRP6; | 342 |
| | SSRP7; SSRP8; SSRP9; SSRP10; SSRP11; HINGE1; SSRP12; | |
| | LINK95; SSRP13; SSRP14; LINK96; SSRP15; SSRP16; CTERM1 | |
| PP80 | SS2; NTERM5; SSRP1; SSRP2; SSRP3; SSRP4; SSRP5; SSRP6; | 343 |
| | SSRP7; SSRP8; SSRP9; SSRP10; SSRP11; HINGE1; SSRP12; | |
| | LINK95; SSRP13; SSRP14; LINK96; SSRP15; SSRP16; CTERM1 | |
| PP81 | SS2; NTERM6; SSRP4; SSRP5; SSRP6; SSRP7; SSRP8; SSRP9; | 344 |
| | SSRP10; SSRP11; HINGE1; SSRP12; LINK95; SSRP13; SSRP14; | |
| | LINK96; SSRP15; SSRP16; CTERM1 | |
| PP82 | SS2; SSRP81; SSRP8; SSRP9; SSRP10; SSRP11; HINGE1; | 345 |
| | SSRP12; LINK95; SSRP13; SSRP14; LINK96; SSRP15; SSRP16; | |
| | CTERM1 | |
| PP83 | SS2; SSRP82; SSRP6; SSRP7; SSRP8; SSRP9; SSRP10; | 346 |
| | SSRP11; HINGE1; SSRP12; LINK95; SSRP13; SSRP14; LINK96; | |
| | SSRP83 | |
| PP84 | SS2; SSRP84; SSRP7; SSRP8; SSRP9; SSRP10; SSRP11; | 347 |
| | HINGE1; SSRP12; LINK95; SSRP13; SSRP14; LINK96; SSRP83 | |
| PP85 | SS2; NTERM1; SSRP1; SSRP2; SSRP3; SSRP4; SSRP5; SSRP6; | 348 |
| | SSRP7; SSRP8; SSRP9; SSRP10; SSRP11; HINGE1; SSRP12; | |
| | SSRP13; SSRP14; SSRP15; SSRP16; CTERM1 | |
| PP86 | SS2; NTERM1; SSRP1; SSRP2; SSRP3; SSRP4; SSRP5; SSRP6; | 349 |
| | SSRP7; SSRP8; SSRP9; SSRP10; SSRP11; SSRP12; LINK95; | |
| | SSRP13; SSRP14; LINK96; SSRP15; SSRP16; CTERM1 | |
| PP87 | SS2; NTERM1; SSRP1; SSRP2; SSRP3; SSRP4; SSRP5; SSRP6; | 350 |
| | SSRP7; SSRP8; SSRP9; SSRP10; SSRP11; SSRP12; SSRP13; | |
| | SSRP14; SSRP15; SSRP16; CTERM1 | |
| PP88 | SS2; NTERM1; SSRP1; SSRP2; SSRP3; SSRP4; SSRP21; | 351 |
| | SSRP6; SSRP7; SSRP8; SSRP22; SSRP10; SSRP11; HINGE1; | |
| | SSRP12; LINK95; SSRP13; SSRP14; LINK96; SSRP15; SSRP16; | |
| | CTERM1 | |
| PP89 | SS2; SSRP32; SSRP33; SSRP34; SSRP35; SSRP36; SSRP37; | 352 |
| | SSRP38; SSRP39; SSRP40; SSRP41; SSRP42; SSRP43; | |
| | SSRP44; SSRP45; SSRP46; SSRP47; SSRP48; CTERM2 | |
| PP90 | SS2; SSRP31; SSRP32; SSRP33; SSRP34; SSRP35; SSRP36; | 353 |
| | SSRP37; SSRP38; SSRP39; SSRP40; SSRP41; SSRP42; | |
| | SSRP43; SSRP44; SSRP45; SSRP46; SSRP47; SSRP48; CTERM2 | |
| PP91 | SS2; SSRP32; SSRP33; SSRP34; SSRP35; SSRP36; SSRP37; | 354 |
| | SSRP7; SSRP8; SSRP9; SSRP41; SSRP42; SSRP85; SSRP44; | |
| | SSRP45; SSRP46; SSRP47; SSRP48; CTERM2 | |
| PP92 | SS2; SSRP31; SSRP32; SSRP33; SSRP34; SSRP35; SSRP36; | 355 |
| | SSRP37; SSRP7; SSRP8; SSRP9; SSRP41; SSRP42; SSRP85; | |
| | SSRP44; SSRP45; SSRP46; SSRP47; SSRP48; CTERM2 | |
| PP93 | SS1; NTERM1; SSRP1; SSRP2; SSRP3; SSRP4; SSRP5; SSRP6; | 356 |
| | SSRP7; SSRP8; SSRP9; SSRP10; SSRP11; HINGE1; SSRP12; | |
| | LINK95; SSRP13; SSRP78; LINK97; SSRP18; SSRP19; CTERM1 | |
| PP94 | SS2; NTERM1; SSRP1; SSRP2; SSRP3; SSRP4; SSRP5; SSRP6; | 357 |
| | SSRP17; SSRP8; SSRP9; SSRP10; SSRP11; HINGE1; SSRP12; | |
| | SSRP13; SSRP14; SSRP15; SSRP16; CTERM1 | |
| PP95 | SS2; NTERM1; SSRP1; SSRP2; SSRP3; SSRP4; SSRP21; | 358 |
| | SSRP6; SSRP7; SSRP8; SSRP87; SSRP10; SSRP11; HINGE1; | |
| | SSRP12; SSRP13; SSRP14; SSRP15; SSRP16; CTERM1 | |
| PP96 | SS2; NTERM1; SSRP1; SSRP2; SSRP3; SSRP4; SSRP5; SSRP6; | 359 |
| | SSRP7; SSRP8; SSRP9; SSRP10; SSRP11; HINGE1; SSRP12; | |
| | SSRP13; SSRP14; SSRP15; SSRP16 | |
| PP97 | SS2; NTERM1; SSRP1; SSRP2; SSRP3; SSRP4; SSRP5; SSRP6; | 360 |
| | SSRP7; SSRP8; SSRP9; SSRP10; SSRP11; HINGE1; SSRP12; | |
| | SSRP13; SSRP78; LINK97; SSRP18; SSRP19; CTERM1 | |
| PP98 | SS2; NTERM1; SSRP1; SSRP2; SSRP3; SSRP4; SSRP5; SSRP6; | 361 |
| | SSRP7; SSRP8; SSRP9; SSRP10; SSRP11; HINGE1; SSRP12; | |
| | SSRP13; SSRP78; SSRP18; SSRP19 | |
| PP99 | SS2; NTERM1; SSRP1; SSRP2; SSRP3; SSRP4; SSRP5; SSRP6; | 362 |
| | SSRP17; SSRP8; SSRP9; SSRP10; SSRP11; HINGE1; SSRP12; | |
| | SSRP13; SSRP78; SSRP18; SSRP19; CTERM1 | |
| PP100 | SS2; NTERM1; SSRP1; SSRP2; SSRP3; SSRP4; SSRP21; | 363 |
| | SSRP6; SSRP7; SSRP8; SSRP22; SSRP10; SSRP11; HINGE1; | |
| | SSRP12; SSRP13; SSRP78; SSRP18; SSRP19; CTERM1 | |
| PP101 | SS2; NTERM1; SSRP1; SSRP2; SSRP3; SSRP4; SSRP5; SSRP6; | 364 |
| | SSRP7; SSRP8; SSRP9; SSRP10; SSRP11; HINGE1; SSRP12; | |
| | SSRP13; SSRP78; SSRP15; SSRP16; CTERM1 | |
| PP102 | SS2; NTERM1; SSRP1; SSRP2; SSRP3; SSRP4; SSRP5; SSRP6; | 365 |
| | SSRP7; SSRP8; SSRP9; SSRP10; SSRP11; HINGE1; SSRP12; | |
| | SSRP13; SSRP14; SSRP18; SSRP16; CTERM1 | |
| PP103 | SS2; NTERM1; SSRP1; SSRP2; SSRP3; SSRP4; SSRP5; SSRP6; | 366 |
| | SSRP7; SSRP8; SSRP9; SSRP10; SSRP11; HINGE1; SSRP12; | |
| | SSRP13; SSRP14; SSRP15; SSRP20; CTERM1 | |
| PP104 | SS2; NTERM1; SSRP1; SSRP2; SSRP3; SSRP4; SSRP5; SSRP6; | 367 |
| | SSRP7; SSRP8; SSRP9; SSRP10; SSRP11; HINGE1; SSRP12; | |
| | SSRP13; SSRP14; SSRP15; SSRP79; CTERM1 | |
| PP105 | SS2; SSRP32; SSRP1; SSRP2; SSRP3; SSRP4; SSRP5; SSRP6; | 368 |
| | SSRP7; SSRP8; SSRP9; SSRP10; SSRP11; SSRP12; SSRP13; | |
| | SSRP14; SSRP15; SSRP16; CTERM1 | |
| PP106 | SS2; SSRP32; SSRP33; SSRP1; SSRP2; SSRP3; SSRP4; SSRP5; | 369 |
| | SSRP6; SSRP7; SSRP8; SSRP9; SSRP10; SSRP11; SSRP12; | |
| | SSRP13; SSRP14; SSRP15; SSRP16; CTERM1 | |
| PP107 | SS2; SSRP32; SSRP33; SSRP34; SSRP1; SSRP2; SSRP3; | 370 |
| | SSRP4; SSRP5; SSRP6; SSRP7; SSRP8; SSRP9; SSRP10; | |
| | SSRP11; SSRP12; SSRP13; SSRP14; SSRP15; SSRP16; CTERM1 | |
| PP108 | SS2; SSRP32; SSRP33; SSRP34; SSRP1; SSRP2; SSRP3; | 371 |
| | SSRP4; SSRP5; SSRP6; SSRP7; SSRP8; SSRP9; SSRP10; | |
| | SSRP11; SSRP12; SSRP13; SSRP14; SSRP15; SSRP16 | |
| PP109 | SS2; SSRP32; SSRP33; SSRP34; SSRP1; SSRP2; SSRP3; | 372 |
| | SSRP4; SSRP5; SSRP6; SSRP7; SSRP8; SSRP9; SSRP10; | |
| | SSRP11; SSRP12; SSRP13; SSRP78; SSRP18; SSRP19 | |
| PP110 | SS2; NTERM1; SSRP1; SSRP2; SSRP3; SSRP4; SSRP5; SSRP6; | 373 |
| | SSRP7; SSRP8; SSRP9; SSRP10; SSRP11; HINGE1; SSRP80; | |
| | LINK95; SSRP13; SSRP14; LINK96; SSRP15; SSRP16; CTERM1 | |
| PP111 | SS15; NTERM2; SSRP31; SSRP32; SSRP33; SSRP34; SSRP35; | 9 |
| | SSRP36; SSRP37; SSRP38; SSRP39; SSRP40; SSRP41; | |
| | SSRP42; SSRP43; SSRP44; SSRP45; SSRP46; SSRP47; | |
| | SSRP48; CTERM2 | |
| PP112 | SS15; NTERM2; SSRP31; SSRP32; SSRP33; SSRP34; SSRP35; | 374 |
| | SSRP36; SSRP37; SSRP38; SSRP39; SSRP40; SSRP41; | |
| | SSRP86; SSRP43; SSRP44; SSRP45; SSRP46; SSRP47; | |
| | SSRP48; CTERM2 | |
| PP113 | SS2; NTERM1; SSRP1; SSRP2; SSRP3; SSRP4; SSRP5; SSRP6; | 375 |
| | SSRP7; SSRP8; SSRP9; SSRP10; SSRP11; HINGE1; SSRP80; | |
| | SSRP13; SSRP14; SSRP15; SSRP16; CTERM1 | |
| PP114 | SS2; NTERM1; SSRP1; SSRP2; SSRP3; SSRP4; SSRP5; SSRP6; | 376 |
| | SSRP17; SSRP8; SSRP9; SSRP10; SSRP11; HINGE1; SSRP80; | |
| | SSRP13; SSRP14; SSRP15; SSRP16; CTERM1 | |
| PP115 | SS2; NTERM1; SSRP1; SSRP2; SSRP3; SSRP4; SSRP5; SSRP6; | 377 |
| | SSRP17; SSRP8; SSRP9; SSRP10; SSRP11; HINGE1; SSRP12; | |
| | SSRP13; SSRP14; SSRP15; SSRP20; CTERM1 | |
| PP116 | SS2; NTERM1; SSRP1; SSRP2; SSRP3; SSRP4; SSRP5; SSRP6; | 378 |
| | SSRP7; SSRP8; SSRP9; SSRP10; SSRP11; HINGE1; SSRP80; | |
| | SSRP13; SSRP14; SSRP15; SSRP20; CTERM1 | |
| PP117 | SS2; NTERM1; SSRP1; SSRP2; SSRP3; SSRP4; SSRP5; SSRP6; | 379 |
| | SSRP17; SSRP8; SSRP9; SSRP10; SSRP11; HINGE1; SSRP80; | |
| | SSRP13; SSRP14; SSRP15; SSRP20; CTERM1 | |
| PP118 | SS2; NTERM1; SSRP1; SSRP2; SSRP3; SSRP4; SSRP5; SSRP6; | 380 |
| | SSRP17; SSRP8; SSRP9; SSRP10; SSRP11; SSRP12; SSRP13; | |
| | SSRP14; SSRP15; SSRP16; CTERM1 | |
| PP127 | SS2; NTERM1; SSRP1; SSRP2; SSRP3; SSRP4; SSRP5; SSRP6; | 381 |
| | SSRP7; SSRP8; SSRP9; SSRP10; SSRP11; SSRP80; SSRP13; | |
| | SSRP14; SSRP15; SSRP16; CTERM1 | |
| PP120 | SS2; NTERM1; SSRP1; SSRP2; SSRP3; SSRP4; SSRP5; SSRP6; | 382 |
| | SSRP17; SSRP8; SSRP9; SSRP10; SSRP11; SSRP80; SSRP13; | |
| | SSRP14; SSRP15; SSRP16; CTERM1 | |
| PP121 | SS2; NTERM1; SSRP1; SSRP2; SSRP3; SSRP4; SSRP5; SSRP6; | 383 |
| | SSRP17; SSRP8; SSRP9; SSRP10; SSRP11; SSRP12; SSRP13; | |
| | SSRP14; SSRP15; SSRP20; CTERM1 | |
| PP122 | SS2; NTERM1; SSRP1; SSRP2; SSRP3; SSRP4; SSRP5; SSRP6; | 384 |
| | SSRP7; SSRP8; SSRP9; SSRP10; SSRP11; SSRP80; SSRP13; | |
| | SSRP14; SSRP15; SSRP20; CTERM1 | |
| PP123 | SS2; NTERM1; SSRP1; SSRP2; SSRP3; SSRP4; SSRP5; SSRP6; | 385 |
| | SSRP17; SSRP8; SSRP9; SSRP10; SSRP11; SSRP80; SSRP13; | |
| | SSRP14; SSRP15; SSRP20; CTERM1 | |
| PP124 | NTERM1; SSRP1; SSRP2; SSRP3; SSRP4; SSRP5; SSRP6; | 876 |
| | SSRP7; SSRP8; SSRP9; SSRP10; SSRP11; HINGE1; SSRP80; | |
| | SSRP13; SSRP14; SSRP15; SSRP16; CTERM1 | |
| PP125 | NTERM1; SSRP1; SSRP2; SSRP3; SSRP4; SSRP5; SSRP6; | 877 |
| | SSRP17; SSRP8; SSRP9; SSRP10; SSRP11; HINGE1; SSRP80; | |
| | SSRP13; SSRP14; SSRP15; SSRP16; CTERM1 | |
| PP126 | NTERM1; SSRP1; SSRP2; SSRP3; SSRP4; SSRP5; SSRP6; | 878 |
| | SSRP17; SSRP8; SSRP9; SSRP10; SSRP11; SSRP12; SSRP13; | |
| | SSRP14; SSRP15; SSRP16; CTERM1 | |
| PP127 | NTERM1; SSRP1; SSRP2; SSRP3; SSRP4; SSRP5; SSRP6; | 879 |
| | SSRP7; SSRP8; SSRP9; SSRP10; SSRP11; SSRP80; SSRP13; | |
| | SSRP14; SSRP15; SSRP16; CTERM1 | |
| PP128 | NTERM1; SSRP1; SSRP2; SSRP3; SSRP4; SSRP5; SSRP6; | 880 |
| | SSRP17; SSRP8; SSRP9; SSRP10; SSRP11; SSRP80; SSRP13; | |
| | SSRP14; SSRP15; SSRP16; CTERM1 | |
| PP129 | NTERM1; SSRP1; SSRP2; SSRP3; SSRP4; SSRP5; SSRP6; | 881 |
| | SSRP17; SSRP8; SSRP9; SSRP10; SSRP11; SSRP80; SSRP13; | |
| | SSRP14; SSRP15; SSRP20; CTERM1 | |

### Lipid

Compositions of the disclosure may include one or more lipid. As used herein, the term “lipid,” refers to molecules of biological origin that are soluble in organic solvents (e.g., chloroform) but show little to no solubility in water. In some embodiments, the compositions of the disclosure may include a derivative of a lipid. As used herein, the term “derivative” refers to a molecule that has been modified or changed in any way relative to a reference molecule or starting molecule e.g., a lipid.

In some embodiments, the lipid may be a fatty acid, a steroid or a sterol ester. In some embodiments, the fatty acid may be a free fatty acid, a wax, a triacylglycerol, a glycerophospholipid, a sphingolipid, a cardiolipin, or a glycerosphingolipid. In some embodiments, the lipid may be a cationic lipid, fusogenic lipid, an anionic lipid, a neutral lipid, or any combination thereof.

Organ selectivity of the compositions of the disclosure may be achieved by adjusting the proportion of the lipid, for example, to target to the spleen or lungs.

The term “lipid,” as used herein, is meant to embrace all stereoisomers, geometric isomers, tautomers, and isotopes of a depicted or described structure associated with the lipid. When referring to the lipid features or substituents, the terms “optional” or “optionally” refer to a feature or substituent that may or may not occur. For example, “optionally substituted alkyl” encompasses both “alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable. In some embodiments, isotopes of lipids may be utilized to facilitate their detection using mass spectrometry and/or to slow the oxidation rate of the lipid (see Shchepinov, M. S. (2007). Rejuvenation research, 10(1), 47-60; the contents of which are herein incorporated by reference in their entirety).

The lipid described herein may have asymmetric centers, geometric centers (e.g., double bond), or both. All chiral, diastereomeric, racemic forms and all geometric isomeric forms of a structure are intended, unless the specific stereochemistry or isomeric form is specifically indicated. The Lipid of the present disclosure containing an asymmetrically substituted atom may be isolated in optically active or racemic forms. It is well known in the art how to prepare optically active forms, such as by resolution of racemic forms, by synthesis from optically active starting materials, or through use of chiral auxiliaries. Geometric isomers of olefins, C═N double bonds, or other types of double bonds may be present in the lipid described herein, and all such stable isomers are included in the present disclosure. Specifically, cis and trans geometric isomers of the lipid of the present disclosure may also exist and may be isolated as a mixture of isomers or as separated isomeric forms.

Lipids described herein also embrace tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Lipids described herein also embrace all the isotopes of the atoms occurring in the intermediate or final compounds. “Isotopes” refers to atoms having the same atomic number but different mass numbers resulting from a different number of neutrons in the nuclei. Thus, by way of example, each individual hydrogen atom present in formula (200) may be present as a 1H, 2H (deuterium) or 3H (tritium) atom, preferably 1H or 2H. Similarly, by way of example, each individual carbon atom present in formula (200) may be present as a ¹²C, ¹³C or ¹⁴C atom.

In some embodiments, the compositions of the disclosure may include derivative of any of the lipids described herein (herein referred to herein as a “lipid derivative”. As used herein, the term “derivative” refers to a form or a version of a compound e.g., a lipid, that differs from a parent compound e.g., a lipid by at least one atom or a group.

In some embodiments, the lipid derivative may include a hydrophilic group. The term “hydrophilic” and its grammatical equivalents as used herein refers to substances or structures that have polar groups that readily interact with water. In some embodiments, the lipid may include a hydrophobic group. The term “hydrophobic” and its grammatical equivalents as used herein refers to substances or structures that have polar groups that do not readily interact with water. For the structures provided herein, the following parenthetical subscripts further define the groups as follows: “(Cn)” defines the exact number (n) of carbon atoms in the group. For example, “(C₂₋₁₀) alkyl designates those alkyl groups having from 2 to 10 carbon atoms (e.g., 2, 3, 4, 5, 6, 7, 8, 9, or 10, or any range derivable therein (e.g., 3 to 10 carbon atoms). In some embodiments, the lipid derivative may defer from a parent lipid by the inclusion of an alkyl group. An “alkyl” group may refer to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. In some embodiments, the lipid derivative may include an alkene moiety. An “alkene” moiety refers to a group consisting of at least two carbon atoms and at least one carbon-carbon double bond. In some embodiments, the lipid derivative may include alkyne moiety. As used herein, an “alkyne” moiety refers to a group consisting of at least two carbon atoms and at least one carbon-carbon triple bond. The alkyl moiety, whether saturated or unsaturated, may be branched, straight chain, or cyclic. Furthermore, the alkyl moiety, whether saturated or unsaturated, may include branched, straight chain, and/or cyclic portions. Depending on the structure, an alkyl group may be a monoradical or a diradical (i.e., an alkylene group). In some embodiments, the lipid derivative may include a heteroalkyl group. A “heteroalkyl” group is as described for “alkyl” with at least one of the C atoms thereof substituted with an N, S, or O atom. The “heteroalkyl” group may include linear, branched, and/or cyclic portions. In certain embodiments, a “lower alkyl” is an alkyl group with 1-6 carbon atoms (i.e., a C₁-C₆ alkyl group). In specific instances, the “lower alkyl” may be straight chained or branched.

In some embodiments, the lipid derivative may include an aryl group. In some aspects, the lipid derivative may be an aryl radical. “Aryl radical” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) p-electron system in accordance with the Huckel theory. The ring system from which aryl groups are derived includes groups such as benzene, fluorene, indane, indene, tetralin, and naphthalene. In some embodiments, the term “aryl” can refer to an aromatic ring wherein each of the atoms forming the ring is a carbon atom. Aryl rings can be formed by five, six, seven, eight, nine, or more than nine carbon atoms. Aryl groups in the lipid can be optionally substituted. Aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

In some embodiments, the lipid derivates may include heteroaryl moieties. “Heteroaryl radical” refers to a radical derived from a 3- to 12-membered aromatic ring radical that includes two to eleven carbon atoms and at least one heteroatom wherein each heteroatom may be selected from N, O, and S. As used herein, the heteroaryl ring may be selected from monocyclic or bicyclic and fused or bridged ring systems rings wherein at least one of the rings in the ring system is aromatic, i.e., it contains a cyclic, delocalized (4n+2) p-electron system in accordance with the Huckel theory. The heteroatom(s) in the heteroaryl radical may be optionally oxidized. In some embodiments, lipid derivatives may include one or more nitrogen atoms, if present, are optionally quaternized. The heteroaryl may be attached to the rest of the molecule through any atom of the heteroaryl, valence permitting, such as a carbon or nitrogen atom of the heteroaryl. Heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e., thienyl). An “X-membered heteroaryl” refers to the number of endocyclic atoms, i.e., X, in the ring. For example, a 5-membered heteroaryl ring or 5-membered aromatic heterocycle has 5 endocyclic atoms, e.g., triazole, oxazole, thiophene, etc.

In some embodiments, the term “heteroaryl” when used without the “substituted” modifier refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group consists of no atoms other than carbon, hydrogen, aromatic nitrogen, aromatic oxygen and aromatic sulfur. Non-limiting examples of heteraryl groups include acridinyl, furanyl, imidazoimidazolyl, imidazopyrazolyl, imidazopyridinyl, imidazopyrimidinyl, indolyl, indazolinyl, methylpyridyl, oxazolyl, phenylimidazolyl, pyridyl, pyrrolyl, pyrimidyl, pyrazinyl, quinolyl, quinazolyl, quinoxalinyl, tetrahydroquinolinyl, thienyl, triazinyl, pyrrolopyridinyl, pyrrolopyrimidinyl, pyrrolopyrazinyl, pyrrolotriazinyl, pyrroloimidazolyl, chromenyl (where the point of attachment is one of the aromatic atoms), and chromanyl (where the point of attachment is one of the aromatic atoms). Substituted heteroaryl refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group further has at least one atom independently selected from the group consisting of non-aromatic nitrogen, non-aromatic oxygen, non-aromatic sulfur F, Cl, Br, I, Si, and P.

In some embodiments, any of the moieties in the lipids may be substituted. The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)tOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

In some embodiments, the lipid of the disclosure may be modified. Modifications may be performed to alter properties of the lipid and/or the compositions of the disclosure. For example, modifications may be performed to enhance specificity e.g., to a particular target tissue, to change potency, improve rate and extent of absorption, reduce toxicity, change physical or chemical properties (e.g., solubility) to provide desired features.

In some embodiments, the lipid of the disclosure may be modified to include polar functional groups, such as the alcohol, amine, amide, carboxylic acid, sulfonic acid and phosphate groups, which either ionize or are capable of relatively strong intermolecular forces of attraction with water (hydrogen bonding). In some embodiments, weakly polar groups, such as carboxylic acid esters, aryl halides and alkyl halides, may incorporated into the lipid of the disclosure to enhance lipid solubility.

In some embodiments, the lipid of the disclosure may be modified to include reversibly and irreversibly attached groups. For example, groups that are bound directly to the carbon skeleton of the lipid by C—C, C—O and C—N bonds tend to be irreversible. Alternatively, groups that are linked to the lipid by ester, amide, phosphate, sulfate and glycosidic bonds may typically be more reversible.

In some embodiments, the compositions of the disclosure may include a biologically active lipid. In some embodiments, the compositions may be used to transport a biologically active lipid from one site to another site. In some embodiments, the compositions of the disclosure may be used to transport a biologically active lipid from one site in a subject to another site in the subject.

The lipid may include a fat, a wax, a steroid, a cholesterol, a fat-soluble vitamin, a monoglyceride, a diglyceride, a triglyceride, a phospholipid, a sphingolipid, a glycolipid, a cationic or anionic lipid, a derivatized lipids, etc. In some embodiments, the lipid may form micelles, monolayers, and bilayer membranes. In some embodiments, the lipid may self-assemble in combination with other components to lipoprotein systems.

In some embodiments, the lipid may include one or more hydrophobic groups. The term “hydrophobic group,” as used herein, refers to a chemical moiety that is water-insoluble or repelled by water. Hydrophobic groups include, but are not limited to, long-chain alkanes and fatty acids, fluorocarbons, silicones, certain steroids, such as cholesterol, and polymers including, for example, polystyrene and polyisoprene. In some embodiments, the lipid may include one or more hydrophilic groups. The term “hydrophilic group,” as used herein, refers to a chemical moiety that is water-soluble or attracted to water. Hydrophilic groups include, but are not limited to, alcohols, short-chain carboxylic acids, quaternary amines, sulfonates, phosphates, sugars, and certain polymers such as polyethylene glycol (PEG). In some embodiments, the lipid may be amphiphilic compounds. The term “amphiphilic compound,” as used herein, refers to a compound having both hydrophobic portions and hydrophilic portions. For example, the amphiphilic compounds may have one hydrophilic face and one hydrophobic face. Amphiphilic compounds include, but are not limited to, cholic acid and cholic acid analogs and derivatives, and cholesterol formate. In some embodiments, the compositions of the disclosure include a toxic lipid species. In some embodiments, the compositions of the disclosure include a protective lipid species. In some embodiments, the biologically active lipid includes a polyunsaturated fatty acid, a fat-soluble antioxidant, a cholesteryl ester, or a triglyceride, or any combination thereof. In some embodiments, the compositions of the disclosure include a phospholipid such as 1,2-dimyristoyl-sn-glycero-3-phosphocholine (DMPC) or 1-palmitoyl-2-oleoyl-sn-glycero-3-phosphocholine (POPC), plasmalogens, free cholesterols, cholesteryl esters such as cholesteryl oleate, triglycerides such as triolein, and others. In some embodiments, the compositions of the disclosure include plasmalogens. In some embodiments, the compositions of the disclosure include free cholesterol. In some embodiments, the compositions of the disclosure include a cholesteryl ester. In some embodiments, the cholesteryl ester is cholesteryl oleate. In some embodiments, the compositions of the disclosure include a triglyceride. In some embodiments, the triglyceride may be triolein.

In some embodiments, the lipid provides the membrane environment in which cholesterol may be solubilized. In some embodiments, cholesterol may be actively effluxed by the ABC family of transporters. In some embodiments, cholesterol may be passively absorbed from cell membranes and/or lipid-rich debris. In some embodiments, passive absorption of lipid-rich debris may reduce microglial activation or neuroinflammation. For example, in some embodiments, microglia that phagocytose lipid-rich debris may become laden with lipid droplets, including those enriched with cholesteryl esters. In some embodiments, stimulation of microglial cholesterol efflux by exogenous lipoproteins may enhance the microglial phagocytic capacity. In some embodiments, the relative affinities of the lipoprotein for the extracellular matrix and receptors may tune the balance between cholesterol efflux (lipid removal) and lipid delivery. In some embodiments, cholesterol may be transported between cells (e.g., effluxed from astrocytes and delivered to neurons or oligodendrocytes) to promote cell membrane growth, myelination/remyelination, synaptogenesis, and vesicle release. In some embodiments, another lipid delivered to cells, including a phospholipid, a cholesteryl ester, and a triglyceride, may provide sources of energy and alter the metabolic state of the recipient cell. In some embodiments, delivery of lipid species with monounsaturated fatty acyl chains (e.g., oleate) as well as deuterated poly-unsaturated fatty acyl chains (e.g., DHA deuterated at the bis-allylic position, see, Yang et al. (PNAS, 2016, 113(34): E4966-E4975), the contents of which are herein incorporated by reference) may protect cells from oxidative stress and cell death.

The compositions of the disclosure may include a cationic lipid, a zwitterionic lipid, a neutral lipid and an anionic lipid. A suitable lipid can include a fat, a wax, a steroid, a cholesterol, a fat-soluble vitamin, a monoglyceride, a diglyceride, a phospholipid, a sphingolipid, a glycolipid, a cationic or anionic lipid, a derivatized lipid, etc. In some embodiments, the lipid may be a phospholipid, a lysolipid, cholesterol, a phosphatidylcholine, a phosphatidylethanolamine, a phosphatidylglycerol, a phosphatidylserine, a phosphatidylinositol and a PEGylated lipid.

In some embodiments, phospholipids include, but are not limited to, phosphatidylcholine (PC), phosphatidic acid (PA), phosphatidylethanolamine (PE), phosphatidylglycerol (PG), phosphatidylserine (PS), and phosphatidylinositol (PI), dimyristoyl phosphatidyl choline (DMPC), distearoyl phosphatidyl choline (DSPC), dioleoyl phosphatidyl choline (DOPC), dipalmitoyl phosphatidyl choline (DPPC), dimyristoyl phosphatidyl glycerol (DMPG), distearoyl phosphatidyl glycerol (DSPG), dioleoyl phosphatidyl glycerol (DOPG), dipalmitoyl phosphatidyl glycerol (DPPG), dimyristoyl phosphatidyl serine (DMPS), distearoyl phosphatidyl serine (DSPS), dioleoyl phosphatidyl serine (DOPS), dipalmitoyl phosphatidyl serine (DPPS), dioleoyl phosphatidyl ethanolamine (DOPE), palmitoyloleoylphosphatidylcholine (POPC), palmitoyloleoyl-phosphatidylethanolamine (POPE), palmitoyloleoylphosphatidylserine (POPS), palmitoyloleoylphosphatidylglycerol (POPG), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), 1,2-dielaidoyl-sn-glycero-3-phophoethanolamine (transDOPE), and cardiolipin. In some embodiments, phospholipids may be lysolipids, which contain only one fatty acid moiety bonded to the glycerol subunit via an ester linkage. In some embodiments, lipid extracts, such as egg PC, heart extract, brain extract, liver extract, and soy PC, are also contemplated. In some embodiments, the lipids may include derivatized lipids, such as PEGylated lipids. In some embodiments, derivatized lipids may include, for example, DSPE-PEG2000, cholesterol-PEG2000, DSPE-polyglycerol, or other derivatives generally known in the art.

In some embodiments, the compositions of the disclosure may include steroids, characterized by the presence of a fused, tetracyclic nonane ring system. Steroids include, but are not limited to, cholesterol, cholic acid, progesterone, cortisone, aldosterone, estradiol, testosterone, and dehydroepiandrosterone. In some embodiments, synthetic steroids and derivatives thereof are also contemplated. The compositions as described herein may contain cationic lipids, which contain positively charged functional groups under physiological conditions. Cationic lipids include, but are not limited to, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N-[1-(2,3,-ditetradecyloxy)propyl]-N,N-dimethyl-N-hydroxyethylammonium bromide (DMRIE), N-[1-(2,3,dioleyloxy)propyl]-N,N-dimethyl-N-hydroxy ethylammonium bromide (DORIE), 3β-[N-(N′,N′-dimethylaminoethane)carbamoyl]cholesterol (DC-Chol), dimethyldioctadecylammonium (DDAB) and N,N-dimethyl-2,3-dioleyloxy)propylamine (DODMA).

The compositions as described herein may include anionic lipids, which contain a diacylglycerol backbone attached to a phosphate group and a net negative charge. Anionic lipids include, but are not limited to phosphatidic acid, phosphatidylglycerol (PG), phosphatidylserine (PS), phosphoinositides, phosphatidylinositol, phosphatidylinositol-4-phosphate (Ptdlns(4)P), phosphatidylinositol-4,5-biphosphate (PI(4,5)P2), phosphatidylinositol-3,4,5-triphosphate (Ptdlns(3,4,5)P3), and cardiolipin.

In some embodiments, the compositions as described herein may include a lipid, for example, 1,2-dipalmitoyl-sn-glycero-3-phosphocholine (DPPC), 1,2-ditetradecanoyl-sn-glycero-3-phosphocholine (DMPC), 1-tetradecanoyl-2-hexadecanoyl-sn-glycero-3-phosphocholine (MPPC), 1,2-dioctadecanoyl-sn-glycero-3-phosphocholine (DSPC), 1,2-distearoyl-sn-glycero-3-phosphoethanolamine (DSPE), 1,2-dihexadecanoyl-sn-glycero-3-phosphoethanolamine (DPPE), 1,2-distearoyl-sn-glycero-3-phospho-(1′-rac-glycerol) (DSPG), 1,2-dipalmitoyl-sn-glycero-3-phospho-(1′-rac-glycerol) (DPPG), 1-tetradecanoyl-2-hexadecanoyl-sn-glycero-3-phosphoglycerol (MPPG) and cholesterol. In some embodiments, the compositions as described herein may include a neutral lipid. In some embodiments, the neutral lipid may be any lipid including a simple lipid, a complex lipid, and a derived lipid. Neutral lipids include, but are not limited to, phospholipids, glyceroglycolipids, sphingoglycolipids, sphingoids, and sterols. For instance, examples of a phospholipid include natural or synthetic phospholipids, such as phosphatidylcholines (e.g., soybean phosphatidylcholine, egg yolk phosphatidylcholine (EPC), distearoyl phosphatidylcholine (DSPC), dipalmitoyl phosphatidylcholine (DPPC), palmitoyloleoyl phosphatidylcholine (POPC), dimyristoyl phosphatidylcholine (DMPC), dioleoyl phosphatidylcholine (DOPC), etc.), phosphatidylethanolamines (e.g., distearoyl phosphatidylethanolamine (DSPE), dipalmitoyl phosphatidylethanolamine (DPPE), dioleoyl phosphatidylethanolamine (DOPE), dimyristoyl phosphoethanolamine (DMPE), 16-O-monomethyl PE, 16-O-dimethyl PE, 18-1-trans PE, palmitoyloleoyl-phosphatidylethanolamine (POPE), 1-stearoyl-2-oleoyl-phosphatidylethanolamine (SOPE), etc.), glycerophospholipids (e.g., phosphatidylserine, phosphatidic acid, phosphatidylglycerol, phosphatidylinositol, palmitoyloleoyl phosphatidylglycerol (POPG), lysophosphatidylcholine, etc.), sphingophospholipids (e.g., sphingomyelin, ceramide phosphoethanolamine, ceramide phosphoglycerol, ceramide phosphoglycerophosphate, etc.), glycerophosphonolipids, sphingophosphonolipids, natural lecithins (e.g., egg yolk lecithin, soybean lecithin, etc.), and hydrogenated phospholipids (e.g., hydrogenated soybean phosphatidylcholine etc.).

Examples of the glyceroglycolipid in the neutral lipid include sulfoxyribosyl glyceride, diglycosyl diglyceride, digalactosyl diglyceride, galactosyl diglyceride, and glycosyl diglyceride. Examples of the sphingoglycolipid in the neutral lipid include galactosyl cerebroside, lactosyl cerebroside, and ganglioside. Examples of the sphingoid in the neutral lipid include sphingan, icosasphingan, sphingosine, and derivatives thereof. Examples of the sterol in the neutral lipid include cholesterol, dihydrocholesterol, lanosterol, β-sitosterol, campesterol, stigmasterol, brassicasterol, ergocasterol, fucosterol, and 3β-[N-(N′,N′-dimethylaminoethyl)carbamoyl]cholesterol (DC-Chol).

In some embodiments, the compositions as described herein may include any combination of lipids. In some embodiments, the lipid compositions may be tailored to affect characteristics, such as leakage rates, stability, particle size, zeta potential, protein binding, in vivo circulation, and/or accumulation in tissues or organs. For example, negatively or positively lipids, such as DSPG and/or DOTAP, may be included to affect the surface charge of the lipoprotein systems as described herein. In some embodiments, the lipid compositions may include about ten or fewer types of lipids, or about five or fewer types of lipids, or about three or fewer types of lipids. In some embodiments, the lipid may include at least two, at least three, at least four, at least five, at least six, at least seven, at least eight, at least nine, or at least ten different lipids. In some embodiments, the molar percentage (mol %) of a specific type of lipid present may be from about 0% to about 100%, from about 10% to about 100%, from about 20% to about 100%, from about 30% to about 100%, from about 50% to about 100%, from about 60% to about 100%, or from about 70% to 100%, from about 80% to 100%, from about 90% to 100% of the total lipid present in the compositions as described herein. In some embodiments, the molar percentage (mol %) of a specific type of lipid present may be from about 0% to about 90%, from about 0% to about 80%, from about 0% to about 70%, from about 0% to about 60%, from about 0% to about 50%, from about 0% to about 40%, or from about 0% to 30%, from about 0% to 20%, from about 0% to 10% of the total lipid present in the compositions as described herein.

In some embodiments, the compositions of the disclosure include a lipid. In some embodiments, compositions of the disclosure include fewer than 2, 3, 4, 5, 6, 7, 8, 9, 10, 11, 12, 13, 14, 15, 16, 17, 18, 19, or 20 lipid species. In some embodiments, compositions of the disclosure include 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, or 1-20 lipid species. In some embodiments, compositions of the disclosure include at least 1, 2, 3, 4, 5, 6, 7, 8, 9, 10, 11, 12, 13, 14, 15, 16, 17, 18, 19, or 20 lipid species. In some embodiments, compositions of the disclosure include at most 1, 2, 3, 4, 5, 6, 7, 8, 9, 10, 11, 12, 13, 14, 15, 16, 17, 18, 19, or 20 lipid species. In some embodiments, compositions of the disclosure include 1, 2, 3, 4, 5, 6, 7, 8, 9, 10, 11, 12, 13, 14, 15, 16, 17, 18, 19, or 20 lipid species.

### Fatty Acid

In some embodiments, the lipid of the disclosure may be a fatty acid or a derivative thereof. Fatty acids as described herein, refer to carboxylic acids with long chain hydrocarbon side groups.

In some embodiments, the fatty acid may be a free fatty acid. In some embodiments, the hydrocarbon side groups (or chains) may be from about 4 to about 36 carbons long. In some embodiments, the lipid of the disclosure may be a saturated fatty acid which may include one carbon-carbon single bond. In some embodiments, the lipid of the disclosure may be an unsaturated fatty acid which may include one or more carbon-carbon double bonds. In some embodiments, the unsaturated fatty acid may be a monounsaturated fatty acid containing one carbon-carbon double bond. In some embodiments, lipid of the disclosure may be polyunsaturated fatty acid containing at least two carbon-carbon double bonds. Non-limiting examples of saturated fatty acids include lauric acid, myristic acid, palmitic acid and/or stearic acid. Non-limiting examples of monounsaturated fatty acids include palmitoleic acid, oleic acid, crotonic acid, myristoleic acid, palmitoleic acid, sapienic acid, elaidic acid, vaccenic acid, gadoleic acid, eicosenoic acid, erucic acid, and/or nervonic acid. Non-limiting examples of polyunsaturated fatty acids include linolenic acid, linoleic acid, eicosadienoic acid, docosadienoic acid, pinolenic acid, eleostearic acid, mead acid, eicosatrienoic acid, stearidonic acid, arachidonic acid, eicosatetraenoic acid, adrenic acid, bosseopentaenoic acid, eicosapentaenoic acid, ozubondo acid, sardine acid, tetracosanolpentaenoic, acid, cervonic acid, and/or arachidonic acid.

In one embodiment, the fatty acid is an omega-3 fatty acid. In some embodiments, the omega-3 fatty acid is Docosahexaenoic acid (DHA). In another embodiment, the omega-3 fatty acid is Eicosapentaenoic acid (EPA).

### Wax

In some embodiments, the lipid of the disclosure may be or may include waxes. As used herein, waxes may refer to esters of long chain (from about C₁₄ to about C₃₆) saturated or unsaturated fatty acids with long chain (C₁₆ to about C₃₀) alcohols. Waxes may be water insoluble and/or may be solid at biological temperature. Examples of wax are beeswax, and cetyl palmitate.

### Triacylglycerol

In some embodiments, the lipid of the disclosure may be or may include a triacylglycerol. As used herein, the term triacylglycerol refers to molecules that contain three fatty acids, each in ester linkage with a single glycerol.

The triacylglycerols may include the same fatty acid in all three positions (also referred to as simple triacylglycerols) or may include two or three different fatty acids.

### Glycerophospholipid

In some embodiments, the lipid of the disclosure may be or may include a glycerophospholipid. As used herein, glycerophospholipid may refer to a lipid in which two fatty acids are attached in ester linkage to the first and second carbons of glycerol, and a highly polar or charged group may be attached through a phosphodiester linkage to the third carbon of glycerol. The fatty acids in glycerophospholipids may be any known fatty acid.

The lipid of the present disclosure may be represented by formula (I):

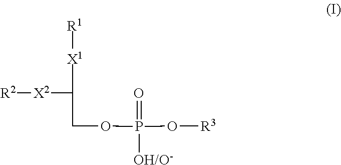

wherein:
X¹ and X² are independently N, O, or absent; and
R¹ and R² are independently optionally substituted acyl, optionally substituted alkyl, optionally substituted alkenyl, or absent.

The glycerophospholipid may be a phosphatidic acid, a phosphatidylethanolamine, a phosphatidylcholine, a phosphatidylserine, or a phosphatidylglycerol.

In some embodiments, the glycerophospholipid may be phosphatidic acid where the group attached to the phosphorous atom in glycerophospholipid is hydroxyl. In some embodiments, the phosphorous atom in glycerophospholipid may be phosphatidylethanolamine, where the group attached to the phosphorous atom in glycerophospholipid may be ethanolamine. In some embodiments, the glycerophospholipid may be phosphatidylcholine, where the group attached to the phosphorous atom in glycerophospholipid may be choline. In some embodiments, the glycerophospholipid may be phosphatidylglycerol, where the group attached to the phosphorous atom in glycerophospholipid may be glycerol. In some embodiments, the glycerophospholipid may be phosphatidyl 4-5-bisphosphate, where the group attached to the phosphorous atom in glycerophospholipid may be myo-inositol 4,5-bisphosphate. In some embodiments, the glycerophospholipid may be cardiolipin, where the group attached to the phosphorous atom in glycerophospholipid may be phosphatidyl glycerol.

In some embodiments, the glycerophospholipid may be an ether lipid or an ether-linked lipid, which may have an alkyl chain attached to the first carbon position by an ether bond. The moiety attached to the phosphate group in ether lipid may be choline, ethanolamine, inositol, or serine. Ether lipid may constitute approximately 20% of the total phospholipid pool in mammals, but tissue distribution varies. The highest levels are found in the brain, heart, spleen, and white blood cells.

The glycerophospholipid may be a derivative of phosphatidic acid, a phosphatidylethanolamine, a phosphatidylcholine, a phosphatidylserine, or a phosphatidylglycerol. In some embodiments, the lipid may be a derivative of derivative of phosphatidylcholine. Phosphatidylcholines may be represented by formula (Ia):

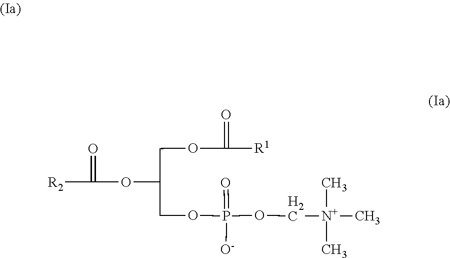

Derivatives of phosphatidylcholine include 1-hexadecanoyl-2-(9Z-octadecenoyl)-sn-glycero-3-phosphocholine (1-palmitoyl-2-oleoyl-sn-glycero-3-phosphocholine or POPC); 1,2-ditetradecanoyl-sn-glycero-3-phosphocholine (1,2-dimyristoyl-sn-glycero-3-phosphocholine or DMPC); 1,2-dihexadecanoyl-sn-glycero-3-phosphocholine (Dipalmitoyl phosphatidylcholine or DPPC); 1,2-di-(9Z-octadecenoyl)-sn-glycero-3-phosphocholine (Dioleoyl phosphatidylcholine or DOPC); 1-hexadecyl-2-(9Z-octadecenoyl)-sn-glycero-3-phosphocholine; 1-O-(1Z-tetradecenyl)-2-(9Z-octadecenoyl)-sn-glycero-3-phosphocholine; 1-dodecanoyl-2-tridecanoyl-sn-glycero-3-phosphocholine; 1-tridecanoyl-sn-glycero-3-phosphocholine; 1-hexadecanoyl-2-(14-pentadecenoyl)-sn-glycero-3-phosphocholine; 1-heneicosanoyl-2-(4Z,7Z,10Z,13Z,16Z,19Z-docosahexaenoyl)-sn-glycero-3-phosphocholine; 1-heptadecanoyl-2-(5Z,8Z,11Z,14Z-eicosatetraenoyl)-sn-glycero-3-phosphocholine; 1,2-dioctadecanoyl-sn-glycero-3-phosphocholine; 1-(6Z,9Z,12Z,15Z-heneicosatetraenoyl)-sn-glycero-3-phosphocholine; 1,2-didecanoyl-sn-glycero-3-phosphocholine; 1-decanoyl-2-octadecanoyl-sn-glycero-3-phosphocholine; 1-decanoyl-2-(9Z-octadecenoyl)-sn-glycero-3-phosphocholine; 1-decanoyl-2-(9Z,12Z-octadecadienoyl)-sn-glycero-3-phosphocholine; 1-decanoyl-2-nonadecanoyl-sn-glycero-3-phosphocholine; 1-decanoyl-2-eicosanoyl-sn-glycero-3-phosphocholine; 1-undecanoyl-2-heptadecanoyl-sn-glycero-3-phosphocholine; 1-undecanoyl-2-nonadecanoyl-sn-glycero-3-phosphocholine; 1-undecanoyl-2-docosanoyl-sn-glycero-3-phosphocholine; 1-undecanoyl-2-tricosanoyl-sn-glycero-3-phosphocholine; 1-undecanoyl-2-pentacosanoyl-sn-glycero-3-phosphocholine; 1-dodecanoyl-sn-glycero-3-phosphocholine; 1,2-didodecanoyl-sn-glycero-3-phosphocholine; 1-dodecanoyl-2-tetradecanoyl-sn-glycero-3-phosphocholine; 1-dodecanoyl-2-hexadecanoyl-sn-glycero-3-phosphocholine; 1-dodecanoyl-2-acetyl-sn-glycero-3-phosphocholine; 1-dodecanoyl-2-eicosanoyl-sn-glycero-3-phosphocholine; 1-dodecanoyl-2-(4Z,7Z,10Z,13Z,16Z,19Z-docosahexaenoyl)-sn-glycero-3-phosphocholine; 1-dodecanoyl-2-tricosanoyl-sn-glycero-3-phosphocholine; 1-dodecanoyl-2-tetracosanoyl-sn-glycero-3-phosphocholine; 1-tridecanoyl-2-pentadecanoyl-sn-glycero-3-phosphocholine; 1-tridecanoyl-2-heptadecanoyl-sn-glycero-3-phosphocholine; 1-tridecanoyl-2-eicosanoyl-sn-glycero-3-phosphocholine; 1-tridecanoyl-2-heneicosanoyl-sn-glycero-3-phosphocholine; 1-tridecanoyl-2-tricosanoyl-sn-glycero-3-phosphocholine; 1-tetradecanoyl-2-dodecanoyl-sn-glycero-3-phosphocholine; 1-tetradecanoyl-2-pentadecanoyl-sn-glycero-3-phosphocholine; 1-tetradecanoyl-2-hexadecanoyl-sn-glycero-3-phosphocholine; 1-tetradecanoyl-2-(9Z-hexadecenoyl)-sn-glycero-3-phosphocholine; 1-tetradecanoyl-2-(11Z-octadecenoyl)-sn-glycero-3-phosphocholine; 1-tetradecanoyl-2-(9Z-octadecenoyl)-sn-glycero-3-phosphocholine; 1-tetradecanoyl-2-(9Z,12Z-octadecadienoyl)-sn-glycero-3-phosphocholine; 1-tetradecanoyl-2-(6Z,9Z,12Z,15Z-octadecatetraenoyl)-sn-glycero-3-phosphocholine; 1-tetradecanoyl-2-acetyl-sn-glycero-3-phosphocholine; 1-tetradecanoyl-2-eicosanoyl-sn-glycero-3-phosphocholine; 1-tetradecanoyl-2-(5Z,8Z,11Z,14Z-eicosatetraenoyl)-sn-glycero-3-phosphocholine; 1-tetradecanoyl-2-(5Z,8Z,11Z,14Z,17Z-eicosapentaenoyl)-sn-glycero-3-phosphocholine; 1-tetradecanoyl-2-(4Z,7Z,10Z,13Z,16Z,19Z-docosahexaenoyl)-sn-glycero-3-phosphocholine; 1-tetradecanoyl-2-hexacosanoyl-sn-glycero-3-phosphocholine; 1-pentadecanoyl-2-tridecanoyl-sn-glycero-3-phosphocholine; 1,2-dipentadecanoyl-sn-glycero-3-phosphocholine; 1-pentadecanoyl-2-heptadecanoyl-sn-glycero-3-phosphocholine; 1-pentadecanoyl-2-nonadecanoyl-sn-glycero-3-phosphocholine; 1-hexadecanoyl-sn-glycero-3-phosphocholine; 1-hexadecanoyl-2-(2E,4E,12E,14E-hexadecatetraenoyl)-sn-glycero-3-phosphocholine; 1-hexadecanoyl-2-(11Z-octadecenoyl)-sn-glycero-3-phosphocholine; 1-hexadecanoyl-2-(6E-octadecenoyl)-sn-glycero-3-phosphocholine; 1-hexadecanoyl-2-(6Z-octadecenoyl)-sn-glycero-3-phosphocholine; 1-hexadecanoyl-2-(9E-octadecenoyl)-sn-glycero-3-phosphocholine; 1-hexadecanoyl-2-(10E,12Z-octadecadienoyl)-sn-glycero-3-phosphocholine; 1-hexadecanoyl-2-(9E,11Z-octadecadienoyl)-sn-glycero-3-phosphocholine; 1-hexadecanoyl-2-(9E,12E-octadecadienoyl)-sn-glycero-3-phosphocholine; 1-hexadecanoyl-2-(9Z,11E,13E,15Z-octadecatetraenoyl)-sn-glycero-3-phosphocholine; 1-hexadecanoyl-2-(5Z,8Z,11Z-eicosatrienoyl)-sn-glycero-3-phosphocholine; 1-hexadecanoyl-2-(8Z,11Z,14Z-eicosatrienoyl)-sn-glycero-3-phosphocholine; 1-hexadecanoyl-2-(5Z,8Z,11Z,14Z,17Z-eicosapentaenoyl)-sn-glycero-3-phosphocholine; 1-hexadecanoyl-2-docosanoyl-sn-glycero-3-phosphocholine; 1-hexadecanoyl-2-(7Z,10Z,13Z,16Z-docosatetraenoyl)-sn-glycero-3-phosphocholine; 1-hexadecanoyl-2-(8E,11E,14E,17E,20E-tricosapentaenoyl)-sn-glycero-3-phosphocholine; 1-hexadecanoyl-2-(15Z-tetracosenoyl)-sn-glycero-3-phosphocholine; 1-hexadecanoyl-2-hexacosanoyl-sn-glycero-3-phosphocholine; 1-hexadecanoyl-2-(5Z,9Z-hexacosadienoyl)-sn-glycero-3-phosphocholine; 1-hexadecanoyl-2-propionyl-sn-glycero-3-phosphocholine; 1-hexadecanoyl-2-(2E-propionyl)-sn-glycero-3-phosphocholine; 1-hexadecanoyl-2-(4E-valeryl)-sn-glycero-3-phosphocholine; 1-hexadecanoyl-2-hexanoyl-sn-glycero-3-phosphocholine; 1-(2Z-hexadecenoyl)-2-(9Z-octadecenoyl)-sn-glycero-3-phosphocholine; 1,2-di-(9E-hexadecenoyl)-sn-glycero-3-phosphocholine; 1-(9Z-hexadecenoyl)-sn-glycero-3-phosphocholine; 1,2-di-(9Z-hexadecenoyl)-sn-glycero-3-phosphocholine; 1-(9Z-hexadecenoyl)-2-(4Z,7Z,10Z,13Z,16Z,19Z-docosahexaenoyl)-sn-glycero-3-phosphocholine; 1-heptadecanoyl-sn-glycero-3-phosphocholine; 1-heptadecanoyl-2-decanoyl-sn-glycero-3-phosphocholine; 1-heptadecanoyl-2-undecanoyl-sn-glycero-3-phosphocholine; 1-heptadecanoyl-2-tridecanoyl-sn-glycero-3-phosphocholine; 1-heptadecanoyl-2-pentadecanoyl-sn-glycero-3-phosphocholine; 1-heptadecanoyl-2-octadecanoyl-sn-glycero-3-phosphocholine; 1-heptadecanoyl-2-(9Z-octadecenoyl)-sn-glycero-3-phosphocholine; 1-heptadecanoyl-2-acetyl-sn-glycero-3-phosphocholine; 1-heptadecanoyl-2-tricosanoyl-sn-glycero-3-phosphocholine; 1-heptadecanoyl-2-nonanoyl-sn-glycero-3-phosphocholine; 1,2-di-(9Z,12Z-heptadecadienoyl)-sn-glycero-3-phosphocholine; 1-octadecanoyl-2-tetradecanoyl-sn-glycero-3-phosphocholine; 1-octadecanoyl-2-(9Z-hexadecenoyl)-sn-glycero-3-phosphocholine; 1-octadecanoyl-2-(11Z-octadecenoyl)-sn-glycero-3-phosphocholine; 1-octadecanoyl-2-(13Z-octadecenoyl)-sn-glycero-3-phosphocholine; 1-octadecanoyl-2-(9E-octadecenoyl)-sn-glycero-3-phosphocholine; 1-octadecanoyl-2-(6Z,9Z-octadecadienoyl)-sn-glycero-3-phosphocholine; 1-octadecanoyl-2-(9Z,12Z-octadecadienoyl)-sn-glycero-3-phosphocholine; 1-octadecanoyl-2-(9E,11E,13E,15E-octadecatetraenoyl)-sn-glycero-3-phosphocholine; 1-octadecanoyl-2-acetyl-sn-glycero-3-phosphocholine; 1-octadecanoyl-2-eicosanoyl-sn-glycero-3-phosphocholine; 1-octadecanoyl-2-(13Z-eicosenoyl)-sn-glycero-3-phosphocholine; 1-octadecanoyl-2-(14Z-eicosenoyl)-sn-glycero-3-phosphocholine; 1-octadecanoyl-2-(5Z-eicosenoyl)-sn-glycero-3-phosphocholine; 1-octadecanoyl-2-(5Z,11Z-eicosadienoyl)-sn-glycero-3-phosphocholine; 1-octadecanoyl-2-(5Z,8Z-eicosadienoyl)-sn-glycero-3-phosphocholine; 1-octadecanoyl-2-(8Z,11Z-eicosadienoyl)-sn-glycero-3-phosphocholine; 1-octadecanoyl-2-(5Z,8Z,14Z-eicosatrienoyl)-sn-glycero-3-phosphocholine; 1-octadecanoyl-2-(5Z,8Z,11Z,14Z-eicosatetraenoyl)-sn-glycero-3-phosphocholine; 1-octadecanoyl-2-(8Z,10Z,12Z,14Z-eicosatetraenoyl)-sn-glycero-3-phosphocholine; 1-octadecanoyl-2-(5Z,8Z,11Z,14Z,17Z-eicosapentaenoyl)-sn-glycero-3-phosphocholine; 1-octadecanoyl-2-(9Z,11Z,13Z,15Z,17Z-eicosapentaenoyl)-sn-glycero-3-phosphocholine; 1-octadecanoyl-2-(7Z,10Z,13Z,16Z,19Z-docosapentaenoyl)-sn-glycero-3-phosphocholine; 1-octadecanoyl-2-(9Z,11Z,13Z,15Z,17Z,19E-docosahexaenoyl)-sn-glycero-3-phosphocholine; 1-octadecanoyl-2-hexacosanoyl-sn-glycero-3-phosphocholine; 1-octadecanoyl-2-propionyl-sn-glycero-3-phosphocholine; 1-octadecanoyl-2-(2E-propionyl)-sn-glycero-3-phosphocholine; 1,2-di-(11E-octadecenoyl)-sn-glycero-3-phosphocholine; 1-(11E-octadecenoyl)-2-docosanoyl-sn-glycero-3-phosphocholine; 1-(11Z-octadecenoyl)-2-octadecanoyl-sn-glycero-3-phosphocholine; 1,2-di-(11Z-octadecenoyl)-sn-glycero-3-phosphocholine; 1-(11Z-octadecenoyl)-2-docosanoyl-sn-glycero-3-phosphocholine; 1-(11Z-octadecenoyl)-2-(7Z,10Z,13Z,16Z,19Z-docosapentaenoyl)-sn-glycero-3-phosphocholine; 1,2-di-(13Z-octadecenoyl)-sn-glycero-3-phosphocholine; 1,2-di-(14Z-octadecenoyl)-sn-glycero-3-phosphocholine; 1,2-di-(16Z-octadecenoyl)-sn-glycero-3-phosphocholine; 1,2-di-(2Z-octadecenoyl)-sn-glycero-3-phosphocholine; 1,2-di-(5Z-octadecenoyl)-sn-glycero-3-phosphocholine; 1,2-di-(8Z-octadecenoyl)-sn-glycero-3-phosphocholine; 1-(9E-octadecenoyl)-sn-glycero-3-phosphocholine; 1-(9Z-octadecenoyl)-sn-glycero-3-phosphocholine; 1-(9Z-octadecenoyl)-2-tetradecanoyl-sn-glycero-3-phosphocholine; 1-(9Z-octadecenoyl)-2-octadecanoyl-sn-glycero-3-phosphocholine; 1-(9Z-octadecenoyl)-2-(6Z,9Z-octadecadienoyl)-sn-glycero-3-phosphocholine; 1-(9Z-octadecenoyl)-2-(9Z,12Z,15Z-octadecatrienoyl)-sn-glycero-3-phosphocholine; 1-(9Z-octadecenoyl)-2-acetyl-sn-glycero-3-phosphocholine; 1-(9Z-octadecenoyl)-2-(5Z,8Z,11Z-eicosatrienoyl)-sn-glycero-3-phosphocholine; 1-(9Z-octadecenoyl)-2-docosanoyl-sn-glycero-3-phosphocholine; 1-(9Z-octadecenoyl)-2-(4Z,7Z,10Z,13Z,16Z,19Z-docosahexaenoyl)-sn-glycero-3-phosphocholine; 1-(9Z-octadecenoyl)-2-tetracosanoyl-sn-glycero-3-phosphocholine; 1-(9Z-octadecenoyl)-2-butyryl-sn-glycero-3-phosphocholine; 1-(2E,4E-octadecadienoyl)-2-hexadecanoyl-sn-glycero-3-phosphocholine; 1-(6Z,9Z-octadecadienoyl)-2-hexadecanoyl-sn-glycero-3-phosphocholine; 1,2-di-(6Z,9Z-octadecadienoyl)-sn-glycero-3-phosphocholine; 1,2-di-(9Z,11Z-octadecadienoyl)-sn-glycero-3-phosphocholine; 1-(9Z,12E-octadecadienoyl)-2-(9Z,11E-heptadecadienoyl)-sn-glycero-3-phosphocholine; 1-(9Z,12Z-octadecadienoyl)-2-hexadecanoyl-sn-glycero-3-phosphocholine; 1-(9Z,12Z-octadecadienoyl)-2-(13Z-eicosenoyl)-sn-glycero-3-phosphocholine; 1-(9Z,12Z-octadecadienoyl)-2-(5Z,8Z,11Z,14Z-eicosatetraenoyl)-sn-glycero-3-phosphocholine; 1-(9Z,12Z-octadecadienoyl)-2-(4Z,7Z,10Z,13Z,16Z,19Z-docosahexaenoyl)-sn-glycero-3-phosphocholine; 1,2-di-(9E,11E,13E-octadecatrienoyl)-sn-glycero-3-phosphocholine; 1-(9Z,12Z,15Z-octadecatrienoyl)-sn-glycero-3-phosphocholine; 1-(9Z,12Z,15Z-octadecatrienoyl)-2-(9Z-octadecenoyl)-sn-glycero-3-phosphocholine; 1,2-di-(9E,11E,13E,15E-octadecatetraenoyl)-sn-glycero-3-phosphocholine; 1,2-di-(9Z,11E,13E,15Z-octadecatetraenoyl)-sn-glycero-3-phosphocholine; 1,2-di-(4Z-octadecenoyl)-sn-glycero-3-phosphocholine; 1-nonadecanoyl-2-undecanoyl-sn-glycero-3-phosphocholine; 1-nonadecanoyl-2-tridecanoyl-sn-glycero-3-phosphocholine; 1-nonadecanoyl-2-tetradecanoyl-sn-glycero-3-phosphocholine; 1-nonadecanoyl-2-pentadecanoyl-sn-glycero-3-phosphocholine; 1-nonadecanoyl-2-nonanoyl-sn-glycero-3-phosphocholine; 1-eicosanoyl-2-dodecanoyl-sn-glycero-3-phosphocholine; 1-eicosanoyl-2-tetradecanoyl-sn-glycero-3-phosphocholine; 1-eicosanoyl-2-(11Z-octadecenoyl)-sn-glycero-3-phosphocholine; 1-eicosanoyl-2-(6Z-octadecenoyl)-sn-glycero-3-phosphocholine; 1-eicosanoyl-2-(7Z-octadecenoyl)-sn-glycero-3-phosphocholine; 1-eicosanoyl-2-(9Z-octadecenoyl)-sn-glycero-3-phosphocholine; 1-eicosanoyl-2-(8Z-eicosenoyl)-sn-glycero-3-phosphocholine; 1-eicosanoyl-2-(11Z,14Z,17Z-eicosatrienoyl)-sn-glycero-3-phosphocholine; 1-eicosanoyl-2-docosanoyl-sn-glycero-3-phosphocholine; 1-eicosanoyl-2-(13Z-docosenoyl)-sn-glycero-3-phosphocholine; 1-eicosanoyl-2-(4Z,7Z,10Z,13Z,16Z,19Z-docosahexaenoyl)-sn-glycero-3-phosphocholine; 1-eicosanoyl-2-hexacosanoyl-sn-glycero-3-phosphocholine; 1-eicosanoyl-2-nonanoyl-sn-glycero-3-phosphocholine; 1,2-di-(11E-eicosenoyl)-sn-glycero-3-phosphocholine; 1,2-di-(11 Z-eicosenoyl)-sn-glycero-3-phosphocholine; 1-(13E-eicosenoyl)-2-docosanoyl-sn-glycero-3-phosphocholine; 1-(9Z-eicosenoyl)-sn-glycero-3-phosphocholine; 1-(5Z,8Z,11Z,14Z-eicosatetraenoyl)-2-hexadecanoyl-sn-glycero-3-phosphocholine; 1-(5Z,8Z,11Z,14Z-eicosatetraenoyl)-2-dotriacontanoyl-sn-glycero-3-phosphocholine; 1-(8E,11E,14E,17E-eicosatetraenoyl)-2-hexadecanoyl-sn-glycero-3-phosphocholine; 1-(5Z,8Z,11Z,14Z,17Z-eicosapentaenoyl)-2-(9Z-octadecenoyl)-sn-glycero-3-phosphocholine; 1-(5Z,8Z,11Z,14Z,17Z-eicosapentaenoyl)-2-(7Z,10Z,3Z,1Z,19Z-docosapentaenoyl)-sn-glycero-3-phosphocholine; 1-(5Z,8Z,11Z,14Z,17Z-eicosapentaenoyl)-2-(4Z,7Z,10Z,13Z,16Z,19Z-docosahexaenoyl)-sn-glycero-3-phosphocholine; 1-heneicosanoyl-2-decanoyl-sn-glycero-3-phosphocholine; 1-heneicosanoyl-2-dodecanoyl-sn-glycero-3-phosphocholine; 1-heneicosanoyl-2-tridecanoyl-sn-glycero-3-phosphocholine; 1-heneicosanoyl-2-octadecanoyl-sn-glycero-3-phosphocholine; 1,2-diheneicosanoyl-sn-glycero-3-phosphocholine; 1-docosanoyl-sn-glycero-3-phosphocholine; 1-docosanoyl-2-decanoyl-sn-glycero-3-phosphocholine; 1-docosanoyl-2-undecanoyl-sn-glycero-3-phosphocholine; 1-docosanoyl-2-octadecanoyl-sn-glycero-3-phosphocholine; 1-docosanoyl-2-(11Z-octadecenoyl)-sn-glycero-3-phosphocholine; 1-docosanoyl-2-(12Z-octadecenoyl)-sn-glycero-3-phosphocholine; 1,2-didocosanoyl-sn-glycero-3-phosphocholine; 1-docosanoyl-2-(13Z-docosenoyl)-sn-glycero-3-phosphocholine; 1-docosanoyl-2-hexacosanoyl-sn-glycero-3-phosphocholine; 1-(4Z,7Z,10Z,13Z,16Z,19Z-docosahexaenoyl)-sn-glycero-3-phosphocholine; 1-(4Z,7Z,10Z,13Z,16Z,19Z-docosahexaenoyl)-2-hexadecanoyl-sn-glycero-3-phosphocholine; 1-tricosanoyl-2-octadecanoyl-sn-glycero-3-phosphocholine; 1,2-ditricosanoyl-sn-glycero-3-phosphocholine; 1-tetracosanoyl-2-octadecanoyl-sn-glycero-3-phosphocholine; 1-tetracosanoyl-2-(6Z-octadecenoyl)-sn-glycero-3-phosphocholine; 1-tetracosanoyl-2-(9Z-octadecenoyl)-sn-glycero-3-phosphocholine; 1,2-ditetracosanoyl-sn-glycero-3-phosphocholine; 1,2-di-(15Z-tetracosenoyl)-sn-glycero-3-phosphocholine; 1-pentacosanoyl-2-octadecanoyl-sn-glycero-3-phosphocholine; 1-propionyl-sn-glycero-3-phosphocholine; 1,2-dipropionyl-sn-glycero-3-phosphocholine; 1-dotriacontanoyl-2-(5Z,8Z,11Z,14Z-eicosatetraenoyl)-sn-glycero-3-phosphocholine; 1-tetratriacontanoyl-2-hexadecanoyl-sn-glycero-3-phosphocholine; 1-hexatriacontanoyl-2-hexadecanoyl-sn-glycero-3-phosphocholine; 1-butyryl-2-octadecanoyl-sn-glycero-3-phosphocholine; 1,2-dibutyryl-sn-glycero-3-phosphocholine; 1-valeryl-2-hexadecanoyl-sn-glycero-3-phosphocholine; 1-hexanoyl-sn-glycero-3-phosphocholine; 1-hexanoyl-2-octadecanoyl-sn-glycero-3-phosphocholine; 1-octanoyl-2-octadecanoyl-sn-glycero-3-phosphocholine; 1-octanoyl-2-eicosanoyl-sn-glycero-3-phosphocholine; 1,2-dioctanoyl-sn-glycero-3-phosphocholine; 1-nonanoyl-2-octadecanoyl-sn-glycero-3-phosphocholine; 1-nonanoyl-2-nonadecanoyl-sn-glycero-3-phosphocholine; 1-nonanoyl-2-eicosanoyl-sn-glycero-3-phosphocholine; 1-nonanoyl-2-heneicosanoyl-sn-glycero-3-phosphocholine; 1,2-dinonanoyl-sn-glycero-3-phosphocholine; 1-tetradecyl-2-hexadecanoyl-sn-glycero-3-phosphocholine; 1-tetradecyl-2-acetyl-sn-glycero-3-phosphocholine; 1-tetradecyl-2-(5Z,8Z,11Z,14Z-eicosatetraenoyl)-sn-glycero-3-phosphocholine; 1-pentadecyl-2-(5Z,8Z,11Z,14Z-eicosatetraenoyl)-sn-glycero-3-phosphocholine; 1-hexadecyl-2-(9Z,12Z-octadecadienoyl)-sn-glycero-3-phosphocholine; 1-hexadecyl-2-(6Z,9Z,12Z-octadecatrienoyl)-sn-glycero-3-phosphocholine; 1-hexadecyl-2-(9Z,12Z,15Z-octadecatrienoyl)-sn-glycero-3-phosphocholine; 1-hexadecyl-2-acetyl-sn-glycero-3-phosphocholine; 1-hexadecyl-2-eicosanoyl-sn-glycero-3-phosphocholine; 1-hexadecyl-2-(5E,8E,11E,14E-eicosatetraenoyl)-sn-glycero-3-phosphocholine; 1-hexadecyl-2-(5Z,8Z,11Z,14Z,17Z-eicosapentaenoyl)-sn-glycero-3-phosphocholine; 1-hexadecyl-2-(7Z,10Z,13Z,16Z,19Z-docosapentaenoyl)-sn-glycero-3-phosphocholine; 1-hexadecyl-2-propionyl-sn-glycero-3-phosphocholine; 1-hexadecyl-2-(2E-propionyl)-sn-glycero-3-phosphocholine; 1-hexadecyl-2-hexanoyl-sn-glycero-3-phosphocholine; 1-(1Z-hexadecenyl)-2-(9Z,12Z-octadecadienoyl)-sn-glycero-3-phosphocholine; 1-heptadecyl-2-heptadecanoyl-sn-glycero-3-phosphocholine; 1-heptadecyl-2-acetyl-sn-glycero-3-phosphocholine; 1-heptadecyl-2-eicosanoyl-sn-glycero-3-phosphocholine; 1-heptadecyl-2-(5Z,8Z,11Z,14Z-eicosatetraenoyl)-sn-glycero-3-phosphocholine; 1-heptadecyl-2-docosanoyl-sn-glycero-3-phosphocholine; 1-octadecyl-2-formyl-sn-glycero-3-phosphocholine; 1-octadecyl-2-(9Z-eicosenoyl)-sn-glycero-3-phosphocholine; 1-octadecyl-2-docosanoyl-sn-glycero-3-phosphocholine; 1-octadecyl-2-(4Z,7Z,10Z,13Z,16Z,19Z-docosahexaenoyl)-sn-glycero-3-phosphocholine; 1-(1Z-octadecenyl)-2-acetyl-sn-glycero-3-phosphocholine; 1-nonadecyl-2-docosanoyl-sn-glycero-3-phosphocholine; 2-tetradecanoyl-sn-glycero-3-phosphocholine; 2-octadecanoyl-sn-glycero-3-phosphocholine; 2-(9E-octadecenoyl)-sn-glycero-3-phosphocholine; 2-(9Z-octadecenoyl)-sn-glycero-3-phosphocholine; 2-(2E-propionyl)-sn-glycero-3-phosphocholine; 2-butyryl-sn-glycero-3-phosphocholine; 2-valeryl-sn-glycero-3-phosphocholine; 2-hexanoyl-sn-glycero-3-phosphocholine; 2-heptanoyl-sn-glycero-3-phosphocholine; 1-(9E-decenyl)-sn-glycero-3-phosphocholine; 1-(1Z-tetradecenyl)-sn-glycero-3-phosphocholine; 1-pentadecyl-sn-glycero-3-phosphocholine; 1-hexadecyl-sn-glycero-3-phosphocholine; 1-(1Z-hexadecenyl)-sn-glycero-3-phosphocholine; 1-(11Z-octadecenyl)-sn-glycero-3-phosphocholine; 1-(1Z-octadecenyl)-sn-glycero-3-phosphocholine; 1-(9Z-octadecenyl)-sn-glycero-3-phosphocholine; 1-nonadecyl-sn-glycero-3-phosphocholine; 1-pentyl-sn-glycero-3-phosphocholine; 1-(9Z-octadecenoyl)-sn-glycero-3-phosphocholine; 1-eicosanoyl-2-(9Z,12Z-octadecadienoyl)-sn-glycero-3-phosphocholine; 1-(7Z-hexadecenoyl)-2-(4Z,7Z,10Z,13Z,16Z,19Z-docosahexaenoyl)-sn-glycero-3-phosphocholine; 1-acyl-sn-glycero-3-phosphocholine; 1-(6-[5]-ladderane-hexanyl)-2-(8-[3]-ladderane-octanyl)-sn-glycerophosphocholine; 1-(8-[5]-ladderane-octanyl)-2-(8-[3]-ladderane-octanyl)-sn-glycerophosphocholine; 1-(8-[3]-ladderane-octanyl)-2-(8-[3]-ladderane-octanyl)-sn-glycerophosphocholine; 1-hexadecanyl-2-(8-[3]-ladderane-octanyl)-sn-glycerophosphocholine; 1-(6-[5]-ladderane-hexanoyl)-2-(8-[3]-ladderane-octanyl)-sn-glycerophosphocholine; 1-(8-[5]-ladderane-octanoyl)-2-(8-[3]-ladderane-octanyl)-sn-glycerophosphocholine; 1-(6-[3]-ladderane-hexanoyl)-2-(8-[3]-ladderane-octanyl)-sn-glycerophosphocholine; 1-(8-[3]-ladderane-octanoyl)-2-(8-[3]-ladderane-octanyl)-sn-glycerophosphocholine; 1-tetradecanoyl-2-(8-[3]-ladderane-octanyl)-sn-glycerophosphocholine; 1-(9E-decenyl)-2-acetyl-sn-glycero-3-phosphocholine; 1-(10E-octadecenyl)-2-acetyl-sn-glycero-3-phosphocholine; 1-(9Z-octadecenyl)-2-(5Z,8Z,11Z,14Z-eicosatetraenoyl)-sn-glycero-3-phosphocholine; 1-(9Z,12Z-octadecadienyl)-2-acetyl-sn-glycero-3-phosphocholine; 1-(2E,6E-phytadienyl)-2-(2E,6E-phytadienyl)-sn-glycero-3-phosphocholine; 1-(2E,6E,10E-phytatrienyl)-2-(2E,6E10E-phytatrienyl)-sn-glycero-3-phosphocholine; 1-(2E-phytaenyl)-2-(2E-phytaenyl)-sn-glycero-3-phosphocholine; 1-(1Z-octadecenyl)-2-(9Z-octadecenoyl)-sn-glycero-3-phosphocholine; 1-(1Z-hexadecenyl)-2-(4Z,7Z,10Z,13Z,16Z,19Z-docosahexaenoyl)-sn-glycero-3-phosphocholine; 1-(7Z,10Z,13Z,16Z-docosatetraenoyl)-glycero-3-phosphocholine; 1-dodecanoyl-2-(9Z-tetradecenoyl)-glycero-3-phosphocholine; 1-dodecanoyl-2-pentadecanoyl-glycero-3-phosphocholine; 1-dodecanoyl-2-(9Z-pentadecenoyl)-glycero-3-phosphocholine; 1-dodecanoyl-2-(9Z-hexadecenoyl)-glycero-3-phosphocholine; 1-dodecanoyl-2-heptadecanoyl-glycero-3-phosphocholine; 1-dodecanoyl-2-(9Z-heptadecenoyl)-glycero-3-phosphocholine; 1-dodecanoyl-2-nonadecanoyl-glycero-3-phosphocholine; 1-dodecanoyl-2-(9Z-nonadecenoyl)-glycero-3-phosphocholine; 1-dodecanoyl-2-(5Z,8Z,11Z,14Z-eicosatetraenoyl)-glycero-3-phosphocholine; 1-dodecanoyl-2-(5Z,8Z,11Z,14Z,17Z-eicosapentaenoyl)-glycero-3-phosphocholine; 1-dodecanoyl-2-heneicosanoyl-glycero-3-phosphocholine; 1-tridecanoyl-2-(9Z-hexadecenoyl)-glycero-3-phosphocholine; 1-tridecanoyl-2-(9Z,12Z-heptadecadienoyl)-glycero-3-phosphocholine; 1-tridecanoyl-2-(6Z,9Z,12Z-octadecatrienoyl)-glycero-3-phosphocholine; 1-tridecanoyl-2-(9Z-nonadecenoyl)-glycero-3-phosphocholine; 1-tridecanoyl-2-(11Z-eicosenoyl)-glycero-3-phosphocholine; 1-tridecanoyl-2-(8Z,11Z,14Z-eicosatrienoyl)-glycero-3-phosphocholine; 1-tridecanoyl-2-(5Z,8Z,11Z,14Z,17Z-eicosapentaenoyl)-glycero-3-phosphocholine; 1-tridecanoyl-2-(11Z-docosenoyl)-glycero-3-phosphocholine; 1-tridecanoyl-2-(13Z,16Z-docosadienoyl)-glycero-3-phosphocholine; 1-tetradecanoyl-2-tridecanoyl-glycero-3-phosphocholine; 1-tetradecanoyl-2-(9Z-pentadecenoyl)-glycero-3-phosphocholine; 1-tetradecanoyl-2-(9Z-heptadecenoyl)-glycero-3-phosphocholine; 1-tetradecanoyl-2-nonadecanoyl-glycero-3-phosphocholine; 1-tetradecanoyl-2-(11Z-eicosenoyl)-glycero-3-phosphocholine; 1-tetradecanoyl-2-(11Z,14Z-eicosadienoyl)-glycero-3-phosphocholine; 1-tetradecanoyl-2-(11Z-docosenoyl)-glycero-3-phosphocholine; 1-tetradecanoyl-2-(13Z,16Z-docosadienoyl)-glycero-3-phosphocholine; 1-(9Z-tetradecenoyl)-2-dodecanoyl-glycero-3-phosphocholine; 1-(9Z-tetradecenoyl)-2-tetradecanoyl-glycero-3-phosphocholine; 1-(9Z-tetradecenoyl)-2-(9Z-pentadecenoyl)-glycero-3-phosphocholine; 1-(9Z-tetradecenoyl)-2-hexadecanoyl-glycero-3-phosphocholine; 1-(9Z-tetradecenoyl)-2-(9Z-hexadecenoyl)-glycero-3-phosphocholine; 1-(9Z-tetradecenoyl)-2-heptadecanoyl-glycero-3-phosphocholine; 1-(9Z-tetradecenoyl)-2-(9Z-heptadecenoyl)-glycero-3-phosphocholine; 1-(9Z-tetradecenoyl)-2-(9Z,12Z-heptadecadienoyl)-glycero-3-phosphocholine; 1-(9Z-tetradecenoyl)-2-octadecanoyl-glycero-3-phosphocholine; 1-(9Z-tetradecenoyl)-2-(9Z,12Z,15Z-octadecatrienoyl)-glycero-3-phosphocholine; 1-(9Z-tetradecenoyl)-2-(6Z,9Z,12Z,15Z-octadecatetraenoyl)-glycero-3-phosphocholine; 1-(9Z-tetradecenoyl)-2-eicosanoyl-glycero-3-phosphocholine; 1-(9Z-tetradecenoyl)-2-(11Z-eicosenoyl)-glycero-3-phosphocholine; 1-(9Z-tetradecenoyl)-2-(11Z,14Z-eicosadienoyl)-glycero-3-phosphocholine; 1-(9Z-tetradecenoyl)-2-(5Z,8Z,11Z,14Z-eicosatetraenoyl)-glycero-3-phosphocholine; 1-(9Z-tetradecenoyl)-2-(13Z,16Z-docosadienoyl)-glycero-3-phosphocholine; 1-(9Z-tetradecenoyl)-2-(4Z,7Z,10Z,13Z,16Z,19Z-docosahexaenoyl)-glycero-3-phosphocholine; 1-pentadecanoyl-2-tetradecanoyl-glycero-3-phosphocholine; 1-pentadecanoyl-2-(9Z-tetradecenoyl)-glycero-3-phosphocholine; 1-pentadecanoyl-2-(9Z-heptadecenoyl)-glycero-3-phosphocholine; 1-pentadecanoyl-2-(9Z-octadecenoyl)-glycero-3-phosphocholine; 1-pentadecanoyl-2-(6Z,9Z,12Z-octadecatrienoyl)-glycero-3-phosphocholine; 1-pentadecanoyl-2-(9Z,12Z,15Z-octadecatrienoyl)-glycero-3-phosphocholine; 1-pentadecanoyl-2-(6Z,9Z,12Z,15Z-octadecatetraenoyl)-glycero-3-phosphocholine; 1-pentadecanoyl-2-(9Z-nonadecenoyl)-glycero-3-phosphocholine; 1-pentadecanoyl-2-(11Z-eicosenoyl)-glycero-3-phosphocholine; 1-pentadecanoyl-2-(11Z,14Z-eicosadienoyl)-glycero-3-phosphocholine; 1-pentadecanoyl-2-(8Z,11Z,14Z-eicosatrienoyl)-glycero-3-phosphocholine; 1-pentadecanoyl-2-(5Z,8Z,11Z,14Z,17Z-eicosapentaenoyl)-glycero-3-phosphocholine; 1-pentadecanoyl-2-docosanoyl-glycero-3-phosphocholine; 1-pentadecanoyl-2-(11Z-docosenoyl)-glycero-3-phosphocholine; 1-pentadecanoyl-2-(4Z,7Z,10Z,13Z,16Z,19Z-docosahexaenoyl)-glycero-3-phosphocholine; 1-(9Z-pentadecenoyl)-2-tridecanoyl-glycero-3-phosphocholine; 1-(9Z-pentadecenoyl)-2-(9Z-tetradecenoyl)-glycero-3-phosphocholine; 1-(9Z-pentadecenoyl)-2-pentadecanoyl-glycero-3-phosphocholine; 1,2-di-(9Z-pentadecenoyl)-sn-glycero-3-phosphocholine; 1-(9Z-pentadecenoyl)-2-hexadecanoyl-glycero-3-phosphocholine; 1-(9Z-pentadecenoyl)-2-(9Z-hexadecenoyl)-glycero-3-phosphocholine; 1-(9Z-pentadecenoyl)-2-(9Z-heptadecenoyl)-glycero-3-phosphocholine; 1-(9Z-pentadecenoyl)-2-octadecanoyl-glycero-3-phosphocholine; 1-(9Z-pentadecenoyl)-2-(9Z,12Z-octadecadienoyl)-glycero-3-phosphocholine; 1-(9Z-pentadecenoyl)-2-(9Z,12Z,15Z-octadecatrienoyl)-glycero-3-phosphocholine; 1-(9Z-pentadecenoyl)-2-eicosanoyl-glycero-3-phosphocholine; 1-(9Z-pentadecenoyl)-2-(11Z,14Z-eicosadienoyl)-glycero-3-phosphocholine; 1-(9Z-pentadecenoyl)-2-(8Z,11Z,14Z-eicosatrienoyl)-glycero-3-phosphocholine; 1-(9Z-pentadecenoyl)-2-(5Z,8Z,11Z,14Z-eicosatetraenoyl)-glycero-3-phosphocholine; 1-(9Z-pentadecenoyl)-2-(5Z,8Z,11Z,14Z,17Z-eicosapentaenoyl)-glycero-3-phosphocholine; 1-(9Z-pentadecenoyl)-2-heneicosanoyl-glycero-3-phosphocholine; 1-(9Z-pentadecenoyl)-2-(13Z,16Z-docosadienoyl)-glycero-3-phosphocholine; 1-hexadecanoyl-2-tridecanoyl-glycero-3-phosphocholine; 1-hexadecanoyl-2-(9Z-tetradecenoyl)-glycero-3-phosphocholine; 1-hexadecanoyl-2-(9Z-nonadecenoyl)-glycero-3-phosphocholine; 1-hexadecanoyl-2-(11Z,14Z-eicosadienoyl)-glycero-3-phosphocholine; 1-hexadecanoyl-2-(11Z-docosenoyl)-glycero-3-phosphocholine; 1-hexadecanoyl-2-(13Z,16Z-docosadienoyl)-glycero-3-phosphocholine; 1-(9Z-hexadecenoyl)-2-dodecanoyl-glycero-3-phosphocholine; 1-(9Z-hexadecenoyl)-2-tetradecanoyl-glycero-3-phosphocholine; 1-(9Z-hexadecenoyl)-2-pentadecanoyl-glycero-3-phosphocholine; 1-(9Z-hexadecenoyl)-2-(9Z-pentadecenoyl)-glycero-3-phosphocholine; 1-(9Z-hexadecenoyl)-2-heptadecanoyl-glycero-3-phosphocholine; 1-(9Z-hexadecenoyl)-2-(9Z,12Z-heptadecadienoyl)-glycero-3-phosphocholine; 1-(9Z-hexadecenoyl)-2-(6Z,9Z,12Z-octadecatrienoyl)-glycero-3-phosphocholine; 1-(9Z-hexadecenoyl)-2-nonadecanoyl-glycero-3-phosphocholine; 1-(9Z-hexadecenoyl)-2-eicosanoyl-glycero-3-phosphocholine; 1-(9Z-hexadecenoyl)-2-(11Z-eicosenoyl)-glycero-3-phosphocholine; 1-(9Z-hexadecenoyl)-2-(11Z,14Z-eicosadienoyl)-glycero-3-phosphocholine; 1-(9Z-hexadecenoyl)-2-docosanoyl-glycero-3-phosphocholine; 1-(9Z-hexadecenoyl)-2-(13Z,16Z-docosadienoyl)-glycero-3-phosphocholine; 1-heptadecanoyl-2-(6Z,9Z,12Z-octadecatrienoyl)-glycero-3-phosphocholine; 1-heptadecanoyl-2-(6Z,9Z,12Z,15Z-octadecatetraenoyl)-glycero-3-phosphocholine; 1-heptadecanoyl-2-nonadecanoyl-glycero-3-phosphocholine; 1-heptadecanoyl-2-(9Z-nonadecenoyl)-glycero-3-phosphocholine; 1-heptadecanoyl-2-(11Z,14Z-eicosadienoyl)-glycero-3-phosphocholine; 1-heptadecanoyl-2-heneicosanoyl-glycero-3-phosphocholine; 1-heptadecanoyl-2-docosanoyl-glycero-3-phosphocholine; 1-heptadecanoyl-2-(11Z-docosenoyl)-glycero-3-phosphocholine; 1-heptadecanoyl-2-(13Z,16Z-docosadienoyl)-glycero-3-phosphocholine; 1-(9Z-heptadecenoyl)-2-hexadecanoyl-glycero-3-phosphocholine; 1-(9Z-heptadecenoyl)-2-(9Z-hexadecenoyl)-glycero-3-phosphocholine; 1-(9Z-heptadecenoyl)-2-heptadecanoyl-glycero-3-phosphocholine; 1-(9Z-heptadecenoyl)-2-(9Z,12Z-heptadecadienoyl)-glycero-3-phosphocholine; 1-(9Z-heptadecenoyl)-2-(6Z,9Z,12Z-octadecatrienoyl)-glycero-3-phosphocholine; 1-(9Z-heptadecenoyl)-2-(6Z,9Z,12Z,15Z-octadecatetraenoyl)-glycero-3-phosphocholine; 1-(9Z-heptadecenoyl)-2-(11Z-eicosenoyl)-glycero-3-phosphocholine; 1-(9Z-heptadecenoyl)-2-heneicosanoyl-glycero-3-phosphocholine; 1-(9Z-heptadecenoyl)-2-docosanoyl-glycero-3-phosphocholine; 1-(9Z-heptadecenoyl)-2-(7Z,10Z,13Z,16Z-docosatetraenoyl)-glycero-3-phosphocholine; 1-(9Z,12Z-heptadecadienoyl)-2-dodecanoyl-glycero-3-phosphocholine; 1-(9Z,12Z-heptadecadienoyl)-2-tridecanoyl-glycero-3-phosphocholine; 1-(9Z,12Z-heptadecadienoyl)-2-tetradecanoyl-glycero-3-phosphocholine; 1-(9Z,12Z-heptadecadienoyl)-2-(9Z-tetradecenoyl)-glycero-3-phosphocholine; 1-(9Z,12Z-heptadecadienoyl)-2-(9Z-pentadecenoyl)-glycero-3-phosphocholine; 1-(9Z,12Z-heptadecadienoyl)-2-heptadecanoyl-glycero-3-phosphocholine; 1-(9Z,12Z-heptadecadienoyl)-2-octadecanoyl-glycero-3-phosphocholine; 1-(9Z,12Z-heptadecadienoyl)-2-(9Z-octadecenoyl)-glycero-3-phosphocholine; 1-(9Z,12Z-heptadecadienoyl)-2-(9Z,12Z-octadecadienoyl)-glycero-3-phosphocholine; 1-(9Z,12Z-heptadecadienoyl)-2-(6Z,9Z,12Z-octadecatrienoyl)-glycero-3-phosphocholine; 1-(9Z,12Z-heptadecadienoyl)-2-(9Z,12Z,15Z-octadecatrienoyl)-glycero-3-phosphocholine; 1-(9Z,12Z-heptadecadienoyl)-2-(6Z,9Z,12Z,15Z-octadecatetraenoyl)-glycero-3-phosphocholine; 1-(9Z,12Z-heptadecadienoyl)-2-(11Z-eicosenoyl)-glycero-3-phosphocholine; 1-(9Z,12Z-heptadecadienoyl)-2-(11Z,14Z-eicosadienoyl)-glycero-3-phosphocholine; 1-(9Z,12Z-heptadecadienoyl)-2-(5Z,8Z,11Z,14Z-eicosatetraenoyl)-glycero-3-phosphocholine; 1-(9Z,12Z-heptadecadienoyl)-2-(4Z,7Z,10Z,13Z,16Z,19Z-docosahexaenoyl)-glycero-3-phosphocholine; 1-octadecanoyl-2-tridecanoyl-glycero-3-phosphocholine; 1-octadecanoyl-2-(9Z-tetradecenoyl)-glycero-3-phosphocholine; 1-octadecanoyl-2-(9Z-pentadecenoyl)-glycero-3-phosphocholine; 1-octadecanoyl-2-(9Z,12Z-heptadecadienoyl)-glycero-3-phosphocholine; 1-octadecanoyl-2-(9Z,12Z,15Z-octadecatrienoyl)-glycero-3-phosphocholine; 1-octadecanoyl-2-nonadecanoyl-glycero-3-phosphocholine; 1-octadecanoyl-2-(9Z-nonadecenoyl)-glycero-3-phosphocholine; 1-octadecanoyl-2-heneicosanoyl-glycero-3-phosphocholine; 1-octadecanoyl-2-(13Z,16Z-docosadienoyl)-glycero-3-phosphocholine; 1-(9Z-octadecenoyl)-2-heptadecanoyl-glycero-3-phosphocholine; 1-(9Z-octadecenoyl)-2-(6Z,9Z,12Z-octadecatrienoyl)-glycero-3-phosphocholine; 1-(9Z-octadecenoyl)-2-(9Z-nonadecenoyl)-glycero-3-phosphocholine; 1-(9Z-octadecenoyl)-2-heneicosanoyl-glycero-3-phosphocholine; 1-(9Z-octadecenoyl)-2-(11 Z-docosenoyl)-glycero-3-phosphocholine; 1-(9Z-octadecenoyl)-2-(13Z,16Z-docosadienoyl)-glycero-3-phosphocholine; 1-(9Z-octadecenoyl)-2-(7Z,10Z,13Z,16Z-docosatetraenoyl)-glycero-3-phosphocholine; 1-(9Z,12Z-octadecadienoyl)-2-dodecanoyl-glycero-3-phosphocholine; 1-(9Z,12Z-octadecadienoyl)-2-(9Z-pentadecenoyl)-glycero-3-phosphocholine; 1-(9Z,12Z-octadecadienoyl)-2-(9Z-octadecenoyl)-glycero-3-phosphocholine; 1-(9Z,12Z-octadecadienoyl)-2-(6Z,9Z,12Z-octadecatrienoyl)-glycero-3-phosphocholine; 1-(9Z,12Z-octadecadienoyl)-2-(9Z,12Z,15Z-octadecatrienoyl)-glycero-3-phosphocholine; 1-(9Z,12Z-octadecadienoyl)-2-nonadecanoyl-glycero-3-phosphocholine; 1-(9Z,12Z-octadecadienoyl)-2-eicosanoyl-glycero-3-phosphocholine; 1-(9Z,12Z-octadecadienoyl)-2-(5Z,8Z,11Z,14Z,17Z-eicosapentaenoyl)-glycero-3-phosphocholine; 1-(9Z,12Z-octadecadienoyl)-2-(7Z,10Z,13Z,16Z-docosatetraenoyl)-glycero-3-phosphocholine; 1-(6Z,9Z,12Z-octadecatrienoyl)-glycero-3-phosphocholine; 1-(6Z,9Z,12Z-octadecatrienoyl)-2-(9Z-tetradecenoyl)-glycero-3-phosphocholine; 1-(6Z,9Z,12Z-octadecatrienoyl)-2-pentadecanoyl-glycero-3-phosphocholine; 1-(6Z,9Z,12Z-octadecatrienoyl)-2-(9Z-pentadecenoyl)-glycero-3-phosphocholine; 1-(6Z,9Z,12Z-octadecatrienoyl)-2-octadecanoyl-glycero-3-phosphocholine; 1-(6Z,9Z,12Z-octadecatrienoyl)-2-(9Z-octadecenoyl)-glycero-3-phosphocholine; 1-(6Z,9Z,12Z-octadecatrienoyl)-2-(6Z,9Z,12Z,15Z-octadecatetraenoyl)-glycero-3-phosphocholine; 1-(6Z,9Z,12Z-octadecatrienoyl)-2-nonadecanoyl-glycero-3-phosphocholine; 1-(6Z,9Z,12Z-octadecatrienoyl)-2-eicosanoyl-glycero-3-phosphocholine; 1-(6Z,9Z,12Z-octadecatrienoyl)-2-(11Z-eicosenoyl)-glycero-3-phosphocholine; 1-(6Z,9Z,12Z-octadecatrienoyl)-2-(11Z,14Z-eicosadienoyl)-glycero-3-phosphocholine; 1-(6Z,9Z,12Z-octadecatrienoyl)-2-(5Z,8Z,11Z,14Z-eicosatetraenoyl)-glycero-3-phosphocholine; 1-(6Z,9Z,12Z-octadecatrienoyl)-2-docosanoyl-glycero-3-phosphocholine; 1-(6Z,9Z,12Z-octadecatrienoyl)-2-(7Z,10Z,13Z,16Z-docosatetraenoyl)-glycero-3-phosphocholine; 1-(6Z,9Z,12Z-octadecatrienoyl)-2-(4Z,7Z,10Z,13Z,16Z,19Z-docosahexaenoyl)-glycero-3-phosphocholine; 1-(9Z,12Z,15Z-octadecatrienoyl)-2-tetradecanoyl-glycero-3-phosphocholine; 1-(9Z,12Z,15Z-octadecatrienoyl)-2-pentadecanoyl-glycero-3-phosphocholine; 1-(9Z,12Z,15Z-octadecatrienoyl)-2-(9Z-heptadecenoyl)-glycero-3-phosphocholine; 1-(9Z,12Z,15Z-octadecatrienoyl)-2-(9Z,12Z-octadecadienoyl)-glycero-3-phosphocholine; 1-(9Z,12Z,15Z-octadecatrienoyl)-2-(6Z,9Z,12Z-octadecatrienoyl)-glycero-3-phosphocholine; 1-(9Z,12Z,15Z-octadecatrienoyl)-2-(6Z,9Z,12Z,15Z-octadecatetraenoyl)-glycero-3-phosphocholine; 1-(9Z,12Z,15Z-octadecatrienoyl)-2-nonadecanoyl-glycero-3-phosphocholine; 1-(9Z,12Z,15Z-octadecatrienoyl)-2-eicosanoyl-glycero-3-phosphocholine; 1-(9Z,12Z,15Z-octadecatrienoyl)-2-(11Z-eicosenoyl)-glycero-3-phosphocholine; 1-(9Z,12Z,15Z-octadecatrienoyl)-2-(11Z,14Z-eicosadienoyl)-glycero-3-phosphocholine; 1-(9Z,12Z,15Z-octadecatrienoyl)-2-heneicosanoyl-glycero-3-phosphocholine; 1-(9Z,12Z,15Z-octadecatrienoyl)-2-docosanoyl-glycero-3-phosphocholine; 1-(9Z,12Z,15Z-octadecatrienoyl)-2-(13Z,16Z-docosadienoyl)-glycero-3-phosphocholine; 1-(9Z,12Z,15Z-octadecatrienoyl)-2-(7Z,10Z,13Z,16Z-docosatetraenoyl)-glycero-3-phosphocholine; 1-(9Z,12Z,15Z-octadecatrienoyl)-2-(4Z,7Z,10Z,13Z,16Z,19Z-docosahexaenoyl)-glycero-3-phosphocholine; 1-(6Z,9Z,12Z,15Z-octadecatetraenoyl)-2-tridecanoyl-glycero-3-phosphocholine; 1-(6Z,9Z,12Z,15Z-octadecatetraenoyl)-2-(9Z-tetradecenoyl)-glycero-3-phosphocholine; 1-(6Z,9Z,12Z,15Z-octadecatetraenoyl)-2-hexadecanoyl-glycero-3-phosphocholine; 1-(6Z,9Z,12Z,15Z-octadecatetraenoyl)-2-heptadecanoyl-glycero-3-phosphocholine; 1-(6Z,9Z,12Z,15Z-octadecatetraenoyl)-2-(9Z,12Z-heptadecadienoyl)-glycero-3-phosphocholine; 1-(6Z,9Z,12Z,15Z-octadecatetraenoyl)-2-(9Z-octadecenoyl)-glycero-3-phosphocholine; 1-(6Z,9Z,12Z,15Z-octadecatetraenoyl)-2-(6Z,9Z,12Z-octadecatrienoyl)-glycero-3-phosphocholine; 1-(6Z,9Z,12Z,15Z-octadecatetraenoyl)-2-nonadecanoyl-glycero-3-phosphocholine; 1-(6Z,9Z,12Z,15Z-octadecatetraenoyl)-2-(9Z-nonadecenoyl)-glycero-3-phosphocholine; 1-(6Z,9Z,12Z,15Z-octadecatetraenoyl)-2-heneicosanoyl-glycero-3-phosphocholine; 1-(6Z,9Z,12Z,15Z-octadecatetraenoyl)-2-(11Z-docosenoyl)-glycero-3-phosphocholine; 1-(6Z,9Z,12Z,15Z-octadecatetraenoyl)-2-(13Z,16Z-docosadienoyl)-glycero-3-phosphocholine; 1-(6Z,9Z,12Z,15Z-octadecatetraenoyl)-2-(4Z,7Z,10Z,13Z,16Z,19Z-docosahexaenoyl)-glycero-3-phosphocholine; 1-nonadecanoyl-2-(9Z-octadecenoyl)-glycero-3-phosphocholine; 1-nonadecanoyl-2-(9Z,12Z-octadecadienoyl)-glycero-3-phosphocholine; 1-nonadecanoyl-2-(6Z,9Z,12Z-octadecatrienoyl)-glycero-3-phosphocholine; 1-nonadecanoyl-2-(6Z,9Z,12Z,15Z-octadecatetraenoyl)-glycero-3-phosphocholine; 1-nonadecanoyl-2-(9Z-nonadecenoyl)-glycero-3-phosphocholine; 1-nonadecanoyl-2-eicosanoyl-glycero-3-phosphocholine; 1-nonadecanoyl-2-(11Z,14Z-eicosadienoyl)-glycero-3-phosphocholine; 1-nonadecanoyl-2-(8Z,11Z,14Z-eicosatrienoyl)-glycero-3-phosphocholine; 1-nonadecanoyl-2-(5Z,8Z,11Z,14Z-eicosatetraenoyl)-glycero-3-phosphocholine; 1-nonadecanoyl-2-docosanoyl-glycero-3-phosphocholine; 1-nonadecanoyl-2-(13Z,16Z-docosadienoyl)-glycero-3-phosphocholine; 1-nonadecanoyl-2-(7Z,10Z,13Z,16Z-docosatetraenoyl)-glycero-3-phosphocholine; 1-nonadecanoyl-2-(4Z,7Z,10Z,13Z,16Z,19Z-docosahexaenoyl)-glycero-3-phosphocholine; 1-(9Z-nonadecenoyl)-glycero-3-phosphocholine; 1-(9Z-nonadecenoyl)-2-(9Z-tetradecenoyl)-glycero-3-phosphocholine; 1-(9Z-nonadecenoyl)-2-pentadecanoyl-glycero-3-phosphocholine; 1-(9Z-nonadecenoyl)-2-(9Z-hexadecenoyl)-glycero-3-phosphocholine; 1-(9Z-nonadecenoyl)-2-(9Z-heptadecenoyl)-glycero-3-phosphocholine; 1-(9Z-nonadecenoyl)-2-(9Z,12Z-heptadecadienoyl)-glycero-3-phosphocholine; 1-(9Z-nonadecenoyl)-2-octadecanoyl-glycero-3-phosphocholine; 1-(9Z-nonadecenoyl)-2-(9Z,12Z-octadecadienoyl)-glycero-3-phosphocholine; 1-(9Z-nonadecenoyl)-2-(6Z,9Z,12Z-octadecatrienoyl)-glycero-3-phosphocholine; 1-(9Z-nonadecenoyl)-2-(6Z,9Z,12Z,15Z-octadecatetraenoyl)-glycero-3-phosphocholine; 1-(9Z-nonadecenoyl)-2-nonadecanoyl-glycero-3-phosphocholine; 1-(9Z-nonadecenoyl)-2-(11Z-eicosenoyl)-glycero-3-phosphocholine; 1-(9Z-nonadecenoyl)-2-(11Z,14Z-eicosadienoyl)-glycero-3-phosphocholine; 1-(9Z-nonadecenoyl)-2-(5Z,8Z,11Z,14Z-eicosatetraenoyl)-glycero-3-phosphocholine; 1-(9Z-nonadecenoyl)-2-(5Z,8Z,11Z,14Z,17Z-eicosapentaenoyl)-glycero-3-phosphocholine; 1-(9Z-nonadecenoyl)-2-docosanoyl-glycero-3-phosphocholine; 1-eicosanoyl-2-(9Z-tetradecenoyl)-glycero-3-phosphocholine; 1-eicosanoyl-2-pentadecanoyl-glycero-3-phosphocholine; 1-eicosanoyl-2-(6Z,9Z,12Z-octadecatrienoyl)-glycero-3-phosphocholine; 1-eicosanoyl-2-(9Z,12Z,15Z-octadecatrienoyl)-glycero-3-phosphocholine; 1-eicosanoyl-2-(5Z,8Z,11Z,14Z,17Z-eicosapentaenoyl)-glycero-3-phosphocholine; 1-eicosanoyl-2-(11Z-docosenoyl)-glycero-3-phosphocholine; 1-eicosanoyl-2-(13Z,16Z-docosadienoyl)-glycero-3-phosphocholine; 1-eicosanoyl-2-(7Z,10Z,13Z,16Z-docosatetraenoyl)-glycero-3-phosphocholine; 1-(11Z-eicosenoyl)-2-dodecanoyl-glycero-3-phosphocholine; 1-(11Z-eicosenoyl)-2-tridecanoyl-glycero-3-phosphocholine; 1-(11Z-eicosenoyl)-2-pentadecanoyl-glycero-3-phosphocholine; 1-(11Z-eicosenoyl)-2-(9Z-pentadecenoyl)-glycero-3-phosphocholine; 1-(11Z-eicosenoyl)-2-hexadecanoyl-glycero-3-phosphocholine; 1-(11Z-eicosenoyl)-2-heptadecanoyl-glycero-3-phosphocholine; 1-(11Z-eicosenoyl)-2-(9Z-heptadecenoyl)-glycero-3-phosphocholine; 1-(11Z-eicosenoyl)-2-(9Z,12Z-heptadecadienoyl)-glycero-3-phosphocholine; 1-(11Z-eicosenoyl)-2-octadecanoyl-glycero-3-phosphocholine; 1-(11Z-eicosenoyl)-2-(9Z-octadecenoyl)-glycero-3-phosphocholine; 1-(11Z-eicosenoyl)-2-(6Z,9Z,12Z,15Z-octadecatetraenoyl)-glycero-3-phosphocholine; 1-(11Z-eicosenoyl)-2-nonadecanoyl-glycero-3-phosphocholine; 1-(11Z-eicosenoyl)-2-(9Z-nonadecenoyl)-glycero-3-phosphocholine; 1-(11Z-eicosenoyl)-2-(11Z,14Z-eicosadienoyl)-glycero-3-phosphocholine; 1-(11Z-eicosenoyl)-2-(5Z,8Z,11Z,14Z-eicosatetraenoyl)-glycero-3-phosphocholine; 1-(11Z-eicosenoyl)-2-(5Z,8Z,11Z,14Z,17Z-eicosapentaenoyl)-glycero-3-phosphocholine; 1-(11Z-eicosenoyl)-2-heneicosanoyl-glycero-3-phosphocholine; 1-(11Z-eicosenoyl)-2-docosanoyl-glycero-3-phosphocholine; 1-(11Z-eicosenoyl)-2-(7Z,10Z,13Z,16Z-docosatetraenoyl)-glycero-3-phosphocholine; 1-(11Z-eicosenoyl)-2-(4Z,7Z,10Z,3Z,1Z,19Z-docosahexaenoyl)-glycero-3-phosphocholine; 1-(11Z,14Z-eicosadienoyl)-2-(9Z-pentadecenoyl)-glycero-3-phosphocholine; 1-(11Z,14Z-eicosadienoyl)-2-(9Z-hexadecenoyl)-glycero-3-phosphocholine; 1-(11Z,14Z-eicosadienoyl)-2-(9Z,12Z-heptadecadienoyl)-glycero-3-phosphocholine; 1-(11Z,14Z-eicosadienoyl)-2-octadecanoyl-glycero-3-phosphocholine; 1-(11Z,14Z-eicosadienoyl)-2-(9Z,12Z-octadecadienoyl)-glycero-3-phosphocholine; 1-(11Z,14Z-eicosadienoyl)-2-(9Z-nonadecenoyl)-glycero-3-phosphocholine; 1-(11Z,14Z-eicosadienoyl)-2-eicosanoyl-glycero-3-phosphocholine; 1-(11Z,14Z-eicosadienoyl)-2-(11Z-eicosenoyl)-glycero-3-phosphocholine; 1-(11Z,14Z-eicosadienoyl)-2-(8Z,11Z,14Z-eicosatrienoyl)-glycero-3-phosphocholine; 1-(11Z,14Z-eicosadienoyl)-2-(5Z,8Z,11Z,14Z-eicosatetraenoyl)-glycero-3-phosphocholine; 1-(11Z,14Z-eicosadienoyl)-2-(11Z-docosenoyl)-glycero-3-phosphocholine; 1-(11Z,14Z-eicosadienoyl)-2-(13Z,16Z-docosadienoyl)-glycero-3-phosphocholine; 1-(8Z,11Z,14Z-eicosatrienoyl)-2-tridecanoyl-glycero-3-phosphocholine; 1-(8Z,11Z,14Z-eicosatrienoyl)-2-tetradecanoyl-glycero-3-phosphocholine; 1-(8Z,11Z,14Z-eicosatrienoyl)-2-(9Z-tetradecenoyl)-glycero-3-phosphocholine; 1-(8Z,11Z,14Z-eicosatrienoyl)-2-(9Z-heptadecenoyl)-glycero-3-phosphocholine; 1-(8Z,11Z,14Z-eicosatrienoyl)-2-(6Z,9Z,12Z-octadecatrienoyl)-glycero-3-phosphocholine; 1-(8Z,11Z,14Z-eicosatrienoyl)-2-(6Z,9Z,12Z,15Z-octadecatetraenoyl)-glycero-3-phosphocholine; 1-(8Z,11Z,14Z-eicosatrienoyl)-2-eicosanoyl-glycero-3-phosphocholine; 1-(8Z,11Z,14Z-eicosatrienoyl)-2-(11Z-eicosenoyl)-glycero-3-phosphocholine; 1-(8Z,11Z,14Z-eicosatrienoyl)-2-heneicosanoyl-glycero-3-phosphocholine; 1-(8Z,11Z,14Z-eicosatrienoyl)-2-(11Z-docosenoyl)-glycero-3-phosphocholine; 1-(8Z,11Z,14Z-eicosatrienoyl)-2-(4Z,7Z,10Z,13Z,16Z,19Z-docosahexaenoyl)-glycero-3-phosphocholine; 1-(5Z,8Z,11Z,14Z-eicosatetraenoyl)-2-dodecanoyl-glycero-3-phosphocholine; 1-(5Z,8Z,11Z,14Z-eicosatetraenoyl)-2-tridecanoyl-glycero-3-phosphocholine; 1-(5Z,8Z,11Z,14Z-eicosatetraenoyl)-2-(9Z-tetradecenoyl)-glycero-3-phosphocholine; 1-(5Z,8Z,11Z,14Z-eicosatetraenoyl)-2-(9Z-pentadecenoyl)-glycero-3-phosphocholine; 1-(5Z,8Z,11Z,14Z-eicosatetraenoyl)-2-(9Z-hexadecenoyl)-glycero-3-phosphocholine; 1-(5Z,8Z,11Z,14Z-eicosatetraenoyl)-2-octadecanoyl-glycero-3-phosphocholine; 1-(5Z,8Z,11Z,14Z-eicosatetraenoyl)-2-(6Z,9Z,12Z-octadecatrienoyl)-glycero-3-phosphocholine; 1-(5Z,8Z,11Z,14Z-eicosatetraenoyl)-2-(9Z,12Z,15Z-octadecatrienoyl)-glycero-3-phosphocholine; 1-(5Z,8Z,11Z,14Z-eicosatetraenoyl)-2-(6Z,9Z,12Z,15Z-octadecatetraenoyl)-glycero-3-phosphocholine; 1-(5Z,8Z,11Z,14Z-eicosatetraenoyl)-2-nonadecanoyl-glycero-3-phosphocholine; 1-(5Z,8Z,11Z,14Z-eicosatetraenoyl)-2-(9Z-nonadecenoyl)-glycero-3-phosphocholine; 1-(5Z,8Z,11Z,14Z-eicosatetraenoyl)-2-(11Z-eicosenoyl)-glycero-3-phosphocholine; 1-(5Z,8Z,11Z,14Z-eicosatetraenoyl)-2-(5Z,8Z,11Z,14Z,17Z-eicosapentaenoyl)-glycero-3-phosphocholine; 1-(5Z,8Z,11Z,14Z-eicosatetraenoyl)-2-heneicosanoyl-glycero-3-phosphocholine; 1-(5Z,8Z,11Z,14Z-eicosatetraenoyl)-2-(11Z-docosenoyl)-glycero-3-phosphocholine; 1-(5Z,8Z,11Z,14Z-eicosatetraenoyl)-2-(4Z,7Z,10Z,13Z,16Z,19Z-docosahexaenoyl)-glycero-3-phosphocholine; 1-(5Z,8Z,11Z,14Z,17Z-eicosapentaenoyl)-2-dodecanoyl-glycero-3-phosphocholine; 1-(5Z,8Z,11Z,14Z,17Z-eicosapentaenoyl)-2-tridecanoyl-glycero-3-phosphocholine; 1-(5Z,8Z,11Z,14Z,17Z-eicosapentaenoyl)-2-pentadecanoyl-glycero-3-phosphocholine; 1-(5Z,8Z,11Z,14Z,17Z-eicosapentaenoyl)-2-(9Z-pentadecenoyl)-glycero-3-phosphocholine; 1-(5Z,8Z,11Z,14Z,17Z-eicosapentaenoyl)-2-(9Z-hexadecenoyl)-glycero-3-phosphocholine; 1-(5Z,8Z,11Z,14Z,17Z-eicosapentaenoyl)-2-(9Z-heptadecenoyl)-glycero-3-phosphocholine; 1-(5Z,8Z,11Z,14Z,17Z-eicosapentaenoyl)-2-(9Z,12Z-heptadecadienoyl)-glycero-3-phosphocholine; 1-(5Z,8Z,11Z,14Z,17Z-eicosapentaenoyl)-2-(9Z-nonadecenoyl)-glycero-3-phosphocholine; 1-(5Z,8Z,11Z,14Z,17Z-eicosapentaenoyl)-2-eicosanoyl-glycero-3-phosphocholine; 1-(5Z,8Z,11Z,14Z,17Z-eicosapentaenoyl)-2-(8Z,11Z,14Z-eicosatrienoyl)-glycero-3-phosphocholine; 1-(5Z,8Z,11Z,14Z,17Z-eicosapentaenoyl)-2-docosanoyl-glycero-3-phosphocholine; 1-(5Z,8Z,11Z,14Z,17Z-eicosapentaenoyl)-2-(11Z-docosenoyl)-glycero-3-phosphocholine; 1-heneicosanoyl-2-hexadecanoyl-glycero-3-phosphocholine; 1-heneicosanoyl-2-(9Z-hexadecenoyl)-glycero-3-phosphocholine; 1-heneicosanoyl-2-heptadecanoyl-glycero-3-phosphocholine; 1-heneicosanoyl-2-(9Z,12Z-heptadecadienoyl)-glycero-3-phosphocholine; 1-heneicosanoyl-2-(9Z-octadecenoyl)-glycero-3-phosphocholine; 1-heneicosanoyl-2-(6Z,9Z,12Z-octadecatrienoyl)-glycero-3-phosphocholine; 1-heneicosanoyl-2-(9Z,12Z,15Z-octadecatrienoyl)-glycero-3-phosphocholine; 1-heneicosanoyl-2-(9Z-nonadecenoyl)-glycero-3-phosphocholine; 1-heneicosanoyl-2-(11Z-eicosenoyl)-glycero-3-phosphocholine; 1-heneicosanoyl-2-(8Z,11Z,14Z-eicosatrienoyl)-glycero-3-phosphocholine; 1-heneicosanoyl-2-(11Z-docosenoyl)-glycero-3-phosphocholine; 1-docosanoyl-2-(9Z-tetradecenoyl)-glycero-3-phosphocholine; 1-docosanoyl-2-pentadecanoyl-glycero-3-phosphocholine; 1-docosanoyl-2-(9Z-pentadecenoyl)-glycero-3-phosphocholine; 1-docosanoyl-2-hexadecanoyl-glycero-3-phosphocholine; 1-docosanoyl-2-(9Z,12Z-heptadecadienoyl)-glycero-3-phosphocholine; 1-docosanoyl-2-(9Z,12Z-octadecadienoyl)-glycero-3-phosphocholine; 1-docosanoyl-2-nonadecanoyl-glycero-3-phosphocholine; 1-docosanoyl-2-(9Z-nonadecenoyl)-glycero-3-phosphocholine; 1-docosanoyl-2-eicosanoyl-glycero-3-phosphocholine; 1-docosanoyl-2-(8Z,11Z,14Z-eicosatrienoyl)-glycero-3-phosphocholine; 1-docosanoyl-2-(5Z,8Z,11Z,14Z-eicosatetraenoyl)-glycero-3-phosphocholine; 1-docosanoyl-2-(4Z,7Z,10Z,13Z,16Z,19Z-docosahexaenoyl)-glycero-3-phosphocholine; 1-(11Z-docosenoyl)-glycero-3-phosphocholine; 1-(11Z-docosenoyl)-2-tetradecanoyl-glycero-3-phosphocholine; 1-(11Z-docosenoyl)-2-(9Z-pentadecenoyl)-glycero-3-phosphocholine; 1-(11Z-docosenoyl)-2-hexadecanoyl-glycero-3-phosphocholine; 1-(11Z-docosenoyl)-2-(9Z-hexadecenoyl)-glycero-3-phosphocholine; 1-(11Z-docosenoyl)-2-(9Z-heptadecenoyl)-glycero-3-phosphocholine; 1-(11Z-docosenoyl)-2-(6Z,9Z,12Z,15Z-octadecatetraenoyl)-glycero-3-phosphocholine; 1-(11Z-docosenoyl)-2-nonadecanoyl-glycero-3-phosphocholine; 1-(11Z-docosenoyl)-2-eicosanoyl-glycero-3-phosphocholine; 1-(11Z-docosenoyl)-2-(11Z,14Z-eicosadienoyl)-glycero-3-phosphocholine; 1-(11Z-docosenoyl)-2-(8Z,11Z,14Z-eicosatrienoyl)-glycero-3-phosphocholine; 1-(11Z-docosenoyl)-2-(5Z,8Z,11Z,14Z,17Z-eicosapentaenoyl)-glycero-3-phosphocholine; 1-(11Z-docosenoyl)-2-docosanoyl-glycero-3-phosphocholine; 1-(11Z-docosenoyl)-2-(4Z,7Z,10Z,13Z,16Z,19Z-docosahexaenoyl)-glycero-3-phosphocholine; 1-(13Z,16Z-docosadienoyl)-2-(9Z-tetradecenoyl)-glycero-3-phosphocholine; 1-(13Z,16Z-docosadienoyl)-2-pentadecanoyl-glycero-3-phosphocholine; 1-(13Z,16Z-docosadienoyl)-2-(9Z-pentadecenoyl)-glycero-3-phosphocholine; 1-(13Z,16Z-docosadienoyl)-2-hexadecanoyl-glycero-3-phosphocholine; 1-(13Z,16Z-docosadienoyl)-2-(9Z-hexadecenoyl)-glycero-3-phosphocholine; 1-(13Z,16Z-docosadienoyl)-2-heptadecanoyl-glycero-3-phosphocholine; 1-(13Z,16Z-docosadienoyl)-2-(9Z,12Z-heptadecadienoyl)-glycero-3-phosphocholine; 1-(13Z,16Z-docosadienoyl)-2-(9Z-octadecenoyl)-glycero-3-phosphocholine; 1-(13Z,16Z-docosadienoyl)-2-(9Z,12Z-octadecadienoyl)-glycero-3-phosphocholine; 1-(13Z,16Z-docosadienoyl)-2-(6Z,9Z,12Z-octadecatrienoyl)-glycero-3-phosphocholine; 1-(13Z,16Z-docosadienoyl)-2-eicosanoyl-glycero-3-phosphocholine; 1-(13Z,16Z-docosadienoyl)-2-(11Z-eicosenoyl)-glycero-3-phosphocholine; 1-(13Z,16Z-docosadienoyl)-2-(11Z,14Z-eicosadienoyl)-glycero-3-phosphocholine; 1-(13Z,16Z-docosadienoyl)-2-(8Z,11Z,14Z-eicosatrienoyl)-glycero-3-phosphocholine; 1-(13Z,16Z-docosadienoyl)-2-heneicosanoyl-glycero-3-phosphocholine; 1-(13Z,16Z-docosadienoyl)-2-docosanoyl-glycero-3-phosphocholine; 1,2-di-(13Z,16Z-docosadienoyl)-sn-glycero-3-phosphocholine; 1-(13Z,16Z-docosadienoyl)-2-(4Z,7Z,10Z,13Z,16Z,19Z-docosahexaenoyl)-glycero-3-phosphocholine; 1-(7Z,10Z,13Z,16Z-docosatetraenoyl)-2-dodecanoyl-glycero-3-phosphocholine; 1-(7Z,10Z,13Z,16Z-docosatetraenoyl)-2-tridecanoyl-glycero-3-phosphocholine; 1-(7Z,10Z,13Z,16Z-docosatetraenoyl)-2-pentadecanoyl-glycero-3-phosphocholine; 1-(7Z,10Z,13Z,16Z-docosatetraenoyl)-2-(9Z-hexadecenoyl)-glycero-3-phosphocholine; 1-(7Z,10Z,13Z,16Z-docosatetraenoyl)-2-heptadecanoyl-glycero-3-phosphocholine; 1-(7Z,10Z,13Z,16Z-docosatetraenoyl)-2-(9Z-heptadecenoyl)-glycero-3-phosphocholine; 1-(7Z,10Z,13Z,16Z-docosatetraenoyl)-2-(9Z,12Z-octadecadienoyl)-glycero-3-phosphocholine; 1-(7Z,10Z,13Z,16Z-docosatetraenoyl)-2-(6Z,9Z,12Z-octadecatrienoyl)-glycero-3-phosphocholine; 1-(7Z,10Z,13Z,16Z-docosatetraenoyl)-2-(6Z,9Z,12Z,15Z-octadecatetraenoyl)-glycero-3-phosphocholine; 1-(7Z,10Z,13Z,16Z-docosatetraenoyl)-2-eicosanoyl-glycero-3-phosphocholine; 1-(7Z,10Z,13Z,16Z-docosatetraenoyl)-2-(11Z-eicosenoyl)-glycero-3-phosphocholine; 1-(7Z,10Z,13Z,16Z-docosatetraenoyl)-2-(11Z,14Z-eicosadienoyl)-glycero-3-phosphocholine; 1-(7Z,10Z,13Z,16Z-docosatetraenoyl)-2-(5Z,8Z,11Z,14Z-eicosatetraenoyl)-glycero-3-phosphocholine; 1-(7Z,10Z,13Z,16Z-docosatetraenoyl)-2-(13Z,16Z-docosadienoyl)-glycero-3-phosphocholine; 1-(4Z,7Z,10Z,13Z,16Z,19Z-docosahexaenoyl)-2-dodecanoyl-glycero-3-phosphocholine; 1-(4Z,7Z,10Z,13Z,16Z,19Z-docosahexaenoyl)-2-tridecanoyl-glycero-3-phosphocholine; 1-(4Z,7Z,10Z,13Z,16Z,19Z-docosahexaenoyl)-2-(9Z-hexadecenoyl)-glycero-3-phosphocholine; 1-(4Z,7Z,10Z,13Z,16Z,19Z-docosahexaenoyl)-2-heptadecanoyl-glycero-3-phosphocholine; 1-(4Z,7Z,10Z,13Z,16Z,19Z-docosahexaenoyl)-2-(9Z-octadecenoyl)-glycero-3-phosphocholine; 1-(4Z,7Z,10Z,13Z,16Z,19Z-docosahexaenoyl)-2-(9Z,12Z-octadecadienoyl)-glycero-3-phosphocholine; 1-(4Z,7Z,10Z,13Z,16Z,19Z-docosahexaenoyl)-2-(6Z,9Z,12Z-octadecatrienoyl)-glycero-3-phosphocholine; 1-(4Z,7Z,10Z,13Z,16Z,19Z-docosahexaenoyl)-2-(9Z,12Z,15Z-octadecatrienoyl)-glycero-3-phosphocholine; 1-(4Z,7Z,10Z,13Z,16Z,19Z-docosahexaenoyl)-2-(6Z,9Z,12Z,15Z-octadecatetraenoyl)-glycero-3-phosphocholine; 1-(4Z,7Z,10Z,13Z,16Z,19Z-docosahexaenoyl)-2-nonadecanoyl-glycero-3-phosphocholine; 1-(4Z,7Z,10Z,13Z,16Z,19Z-docosahexaenoyl)-2-(9Z-nonadecenoyl)-glycero-3-phosphocholine; 1-(4Z,7Z,10Z,13Z,16Z,19Z-docosahexaenoyl)-2-(8Z,11Z,14Z-eicosatrienoyl)-glycero-3-phosphocholine; 1-(4Z,7Z,10Z,13Z,16Z,19Z-docosahexaenoyl)-2-heneicosanoyl-glycero-3-phosphocholine; 1-(4Z,7Z,10Z,13Z,16Z,19Z-docosahexaenoyl)-2-(11Z-docosenoyl)-glycero-3-phosphocholine; 1-hexadecyl-2-dodecanoyl-glycero-3-phosphocholine; 1-hexadecyl-2-tetradecanoyl-glycero-3-phosphocholine; 1-hexadecyl-2-(9Z-tetradecenoyl)-glycero-3-phosphocholine; 1-hexadecyl-2-pentadecanoyl-glycero-3-phosphocholine; 1-hexadecyl-2-(9Z-pentadecenoyl)-glycero-3-phosphocholine; 1-hexadecyl-2-(9Z-hexadecenoyl)-glycero-3-phosphocholine; 1-hexadecyl-2-(9Z,12Z-heptadecadienoyl)-glycero-3-phosphocholine; 1-hexadecyl-2-(6Z,9Z,12Z,15Z-octadecatetraenoyl)-glycero-3-phosphocholine; 1-hexadecyl-2-(9Z-nonadecenoyl)-glycero-3-phosphocholine; 1-hexadecyl-2-(11Z-eicosenoyl)-glycero-3-phosphocholine; 1-hexadecyl-2-(11Z-docosenoyl)-glycero-3-phosphocholine; 1-hexadecyl-2-(13Z,16Z-docosadienoyl)-glycero-3-phosphocholine; 1-octadecyl-2-tridecanoyl-glycero-3-phosphocholine; 1-octadecyl-2-tetradecanoyl-glycero-3-phosphocholine; 1-octadecyl-2-(9Z-tetradecenoyl)-glycero-3-phosphocholine; 1-octadecyl-2-pentadecanoyl-glycero-3-phosphocholine; 1-octadecyl-2-heptadecanoyl-glycero-3-phosphocholine; 1-octadecyl-2-(6Z,9Z,12Z-octadecatrienoyl)-glycero-3-phosphocholine; 1-octadecyl-2-(9Z,12Z,15Z-octadecatrienoyl)-glycero-3-phosphocholine; 1-octadecyl-2-(9Z-nonadecenoyl)-glycero-3-phosphocholine; 1-octadecyl-2-(11Z-eicosenoyl)-glycero-3-phosphocholine; 1-octadecyl-2-(11Z,14Z-eicosadienoyl)-glycero-3-phosphocholine; 1-octadecyl-2-(8Z,11Z,14Z-eicosatrienoyl)-glycero-3-phosphocholine; 1-octadecyl-2-(11Z-docosenoyl)-glycero-3-phosphocholine; 1-eicosyl-glycero-3-phosphocholine; 1-eicosyl-2-tridecanoyl-glycero-3-phosphocholine; 1-eicosyl-2-(9Z-hexadecenoyl)-glycero-3-phosphocholine; 1-eicosyl-2-heptadecanoyl-glycero-3-phosphocholine; 1-eicosyl-2-(9Z-heptadecenoyl)-glycero-3-phosphocholine; 1-eicosyl-2-(9Z,12Z-heptadecadienoyl)-glycero-3-phosphocholine; 1-eicosyl-2-(9Z-octadecenoyl)-glycero-3-phosphocholine; 1-eicosyl-2-(6Z,9Z,12Z,15Z-octadecatetraenoyl)-glycero-3-phosphocholine; 1-eicosyl-2-(9Z-nonadecenoyl)-glycero-3-phosphocholine; 1-eicosyl-2-(11Z,14Z-eicosadienoyl)-glycero-3-phosphocholine; 1-eicosyl-2-(5Z,8Z,11Z,14Z,17Z-eicosapentaenoyl)-glycero-3-phosphocholine; 1-eicosyl-2-heneicosanoyl-glycero-3-phosphocholine; 1-eicosyl-2-(11Z-docosenoyl)-glycero-3-phosphocholine; 1-eicosyl-2-(13Z,16Z-docosadienoyl)-glycero-3-phosphocholine; 1-eicosyl-2-(7Z,10Z,13Z,16Z-docosatetraenoyl)-glycero-3-phosphocholine; 1-eicosyl-2-(4Z,7Z,10Z,13Z,16Z,19Z-docosahexaenoyl)-glycero-3-phosphocholine; 1-(1Z-hexadecenyl)-2-(9Z-tetradecenoyl)-glycero-3-phosphocholine; 1-(1Z-hexadecenyl)-2-(9Z-pentadecenoyl)-glycero-3-phosphocholine; 1-(1Z-hexadecenyl)-2-(9Z-hexadecenoyl)-glycero-3-phosphocholine; 1-(1Z-hexadecenyl)-2-(9Z-heptadecenoyl)-glycero-3-phosphocholine; 1-(1Z-hexadecenyl)-2-octadecanoyl-glycero-3-phosphocholine; 1-(1Z-hexadecenyl)-2-(6Z,9Z,12Z,15Z-octadecatetraenoyl)-glycero-3-phosphocholine; 1-(1Z-hexadecenyl)-2-nonadecanoyl-glycero-3-phosphocholine; 1-(1Z-hexadecenyl)-2-(9Z-nonadecenoyl)-glycero-3-phosphocholine; 1-(1Z-hexadecenyl)-2-eicosanoyl-glycero-3-phosphocholine; 1-(1Z-hexadecenyl)-2-(8Z,11Z,14Z-eicosatrienoyl)-glycero-3-phosphocholine; 1-(1Z-hexadecenyl)-2-heneicosanoyl-glycero-3-phosphocholine; 1-(1Z-hexadecenyl)-2-docosanoyl-glycero-3-phosphocholine; 1-(1Z-hexadecenyl)-2-(11Z-docosenoyl)-glycero-3-phosphocholine; 1-(1Z-hexadecenyl)-2-(13Z,16Z-docosadienoyl)-glycero-3-phosphocholine; 1-(1Z-octadecenyl)-2-dodecanoyl-glycero-3-phosphocholine; 1-(1Z-octadecenyl)-2-tridecanoyl-glycero-3-phosphocholine; 1-(1Z-octadecenyl)-2-tetradecanoyl-glycero-3-phosphocholine; 1-(1Z-octadecenyl)-2-(9Z-tetradecenoyl)-glycero-3-phosphocholine; 1-(1Z-octadecenyl)-2-pentadecanoyl-glycero-3-phosphocholine; 1-(1Z-octadecenyl)-2-heptadecanoyl-glycero-3-phosphocholine; 1-(1Z-octadecenyl)-2-(9Z-heptadecenoyl)-glycero-3-phosphocholine; 1-(1Z-octadecenyl)-2-(9Z,12Z-heptadecadienoyl)-glycero-3-phosphocholine; 1-(1Z-octadecenyl)-2-(9Z,12Z-octadecadienoyl)-glycero-3-phosphocholine; 1-(1Z-octadecenyl)-2-eicosanoyl-glycero-3-phosphocholine; 1-(1Z-octadecenyl)-2-(11Z-eicosenoyl)-glycero-3-phosphocholine; 1-(1Z-octadecenyl)-2-(8Z,11Z,14Z-eicosatrienoyl)-glycero-3-phosphocholine; 1-(1Z-octadecenyl)-2-(5Z,8Z,11Z,14Z,17Z-eicosapentaenoyl)-glycero-3-phosphocholine; 1-(1Z-octadecenyl)-2-docosanoyl-glycero-3-phosphocholine; 1-(1Z-octadecenyl)-2-(11Z-docosenoyl)-glycero-3-phosphocholine; 1-(1Z-octadecenyl)-2-(7Z,10Z,13Z,16Z-docosatetraenoyl)-glycero-3-phosphocholine; 1-(1Z-eicosenyl)-glycero-3-phosphocholine; 1-(1Z-eicosenyl)-2-dodecanoyl-glycero-3-phosphocholine; 1-(1Z-eicosenyl)-2-tridecanoyl-glycero-3-phosphocholine; 1-(1Z-eicosenyl)-2-(9Z-tetradecenoyl)-glycero-3-phosphocholine; 1-(1Z-eicosenyl)-2-pentadecanoyl-glycero-3-phosphocholine; 1-(1Z-eicosenyl)-2-(9Z-hexadecenoyl)-glycero-3-phosphocholine; 1-(1Z-eicosenyl)-2-(9Z-octadecenoyl)-glycero-3-phosphocholine; 1-(1Z-eicosenyl)-2-(6Z,9Z,12Z-octadecatrienoyl)-glycero-3-phosphocholine; 1-(1Z-eicosenyl)-2-(9Z,12Z,15Z-octadecatrienoyl)-glycero-3-phosphocholine; 1-(1Z-eicosenyl)-2-nonadecanoyl-glycero-3-phosphocholine; 1-(1Z-eicosenyl)-2-eicosanoyl-glycero-3-phosphocholine; 1-(1Z-eicosenyl)-2-(11Z-eicosenoyl)-glycero-3-phosphocholine; 1-(1Z-eicosenyl)-2-(8Z,11Z,14Z-eicosatrienoyl)-glycero-3-phosphocholine; 1-(1Z-eicosenyl)-2-(5Z,8Z,11Z,14Z-eicosatetraenoyl)-glycero-3-phosphocholine; 1-(1Z-eicosenyl)-2-heneicosanoyl-glycero-3-phosphocholine; 1-(1Z-eicosenyl)-2-docosanoyl-glycero-3-phosphocholine; 1-(1Z-eicosenyl)-2-(11Z-docosenoyl)-glycero-3-phosphocholine; 1-decanoyl-2-(9Z-hexadecenoyl)-sn-glycero-3-phosphocholine; 1-tetradecanoyl-2-(5Z,8Z,11Z-eicosatrienoyl)-sn-glycero-3-phosphocholine; 1-tetradecanoyl-2-(13Z-docosenoyl)-sn-glycero-3-phosphocholine; 1-tetradecanoyl-2-(7Z,10Z,13Z,16Z,19Z-docosapentaenoyl)-sn-glycero-3-phosphocholine; 1-(9Z-tetradecenoyl)-2-(5Z,8Z,11Z-eicosatrienoyl)-sn-glycero-3-phosphocholine; 1-(9Z-tetradecenoyl)-2-(13Z-docosenoyl)-sn-glycero-3-phosphocholine; 1-(9Z-tetradecenoyl)-2-(4Z,7Z,10Z,13Z,16Z-docosapentaenoyl)-sn-glycero-3-phosphocholine; 1-(9Z-hexadecenoyl)-2-(5Z,8Z,11 Z-eicosatrienoyl)-sn-glycero-3-phosphocholine; 1-(9Z-hexadecenoyl)-2-(4Z,7Z,10Z,13Z,16Z-docosapentaenoyl)-sn-glycero-3-phosphocholine; 1-(11Z-octadecenoyl)-2-tetradecanoyl-sn-glycero-3-phosphocholine; 1-(11Z-octadecenoyl)-2-(9Z-hexadecenoyl)-sn-glycero-3-phosphocholine; 1-(11Z-octadecenoyl)-2-(9Z-octadecenoyl)-sn-glycero-3-phosphocholine; 1-(11Z-octadecenoyl)-2-(6Z,9Z,12Z-octadecatrienoyl)-sn-glycero-3-phosphocholine; 1-(11Z-octadecenoyl)-2-(6Z,9Z,12Z,15Z-octadecatetraenoyl)-sn-glycero-3-phosphocholine; 1-(11Z-octadecenoyl)-2-(11Z,14Z-eicosadienoyl)-sn-glycero-3-phosphocholine; 1-(11Z-octadecenoyl)-2-(8Z,11Z,14Z-eicosatrienoyl)-sn-glycero-3-phosphocholine; 1-(11Z-octadecenoyl)-2-(8Z,11Z,14Z,17Z-eicosatetraenoyl)-sn-glycero-3-phosphocholine; 1-(11Z-octadecenoyl)-2-(13Z,16Z-docosadienoyl)-sn-glycero-3-phosphocholine; 1-(9Z-octadecenoyl)-2-(11Z-octadecenoyl)-sn-glycero-3-phosphocholine; 1-(9Z-octadecenoyl)-2-(8Z,11Z,14Z,17Z-eicosatetraenoyl)-sn-glycero-3-phosphocholine; 1-(9Z,12Z-octadecadienoyl)-2-(11Z-octadecenoyl)-sn-glycero-3-phosphocholine; 1-(9Z,12Z-octadecadienoyl)-2-(5Z,8Z,11Z-eicosatrienoyl)-sn-glycero-3-phosphocholine; 1-(9Z,12Z-octadecadienoyl)-2-(8Z,11Z,14Z,17Z-eicosatetraenoyl)-sn-glycero-3-phosphocholine; 1-(9Z,12Z-octadecadienoyl)-2-(13Z-docosenoyl)-sn-glycero-3-phosphocholine; 1-(6Z,9Z,12Z-octadecatrienoyl)-2-(11Z-octadecenoyl)-sn-glycero-3-phosphocholine; 1-(6Z,9Z,12Z-octadecatrienoyl)-2-(5Z,8Z,11Z-eicosatrienoyl)-sn-glycero-3-phosphocholine; 1-(6Z,9Z,12Z-octadecatrienoyl)-2-(13Z-docosenoyl)-sn-glycero-3-phosphocholine; 1-(6Z,9Z,12Z,15Z-octadecatetraenoyl)-2-(13Z-docosenoyl)-sn-glycero-3-phosphocholine; 1-eicosanoyl-2-(5Z,8Z,11Z-eicosatrienoyl)-sn-glycero-3-phosphocholine; 1-eicosanoyl-2-(8Z,11Z,14Z,17Z-eicosatetraenoyl)-sn-glycero-3-phosphocholine; 1-(11Z-eicosenoyl)-2-(5Z,8Z,11Z-eicosatrienoyl)-sn-glycero-3-phosphocholine; 1-(11Z,14Z-eicosadienoyl)-2-(5Z,8Z,11Z-eicosatrienoyl)-sn-glycero-3-phosphocholine; 1-(5Z,8Z,11Z-eicosatrienoyl)-2-hexadecanoyl-sn-glycero-3-phosphocholine; 1-(5Z,8Z,11Z-eicosatrienoyl)-2-(9Z-hexadecenoyl)-sn-glycero-3-phosphocholine; 1-(5Z,8Z,11Z-eicosatrienoyl)-2-(11Z-octadecenoyl)-sn-glycero-3-phosphocholine; 1-(5Z,8Z,11Z-eicosatrienoyl)-2-(9Z-octadecenoyl)-sn-glycero-3-phosphocholine; 1-(5Z,8Z,11Z-eicosatrienoyl)-2-(9Z,12Z,15Z-octadecatrienoyl)-sn-glycero-3-phosphocholine; 1-(5Z,8Z,11Z-eicosatrienoyl)-2-eicosanoyl-sn-glycero-3-phosphocholine; 1-(5Z,8Z,11Z-eicosatrienoyl)-2-(11Z-eicosenoyl)-sn-glycero-3-phosphocholine; 1-(5Z,8Z,11Z,14Z-eicosatetraenoyl)-2-(11Z-octadecenoyl)-sn-glycero-3-phosphocholine; 1-(8Z,11Z,14Z,17Z-eicosatetraenoyl)-2-tetradecanoyl-sn-glycero-3-phosphocholine; 1-(8Z,11Z,14Z,17Z-eicosatetraenoyl)-2-(9Z-hexadecenoyl)-sn-glycero-3-phosphocholine; 1-(8Z,11Z,14Z,17Z-eicosatetraenoyl)-2-(11Z-eicosenoyl)-sn-glycero-3-phosphocholine; 1-(13Z-docosenoyl)-2-(9Z-tetradecenoyl)-sn-glycero-3-phosphocholine; 1-(13Z-docosenoyl)-2-(9Z-octadecenoyl)-sn-glycero-3-phosphocholine; 1-(13Z-docosenoyl)-2-(6Z,9Z,12Z-octadecatrienoyl)-sn-glycero-3-phosphocholine; 1-(13Z-docosenoyl)-2-(9Z,12Z,15Z-octadecatrienoyl)-sn-glycero-3-phosphocholine; 1-(13Z-docosenoyl)-2-(6Z,9Z,12Z,15Z-octadecatetraenoyl)-sn-glycero-3-phosphocholine; 1-(13Z-docosenoyl)-2-(5Z,8Z,11Z-eicosatrienoyl)-sn-glycero-3-phosphocholine; 1-(13Z-docosenoyl)-2-(8Z,11Z,14Z-eicosatrienoyl)-sn-glycero-3-phosphocholine; 1-(13Z,16Z-docosadienoyl)-2-(11Z-octadecenoyl)-sn-glycero-3-phosphocholine; 1-(7Z,10Z,13Z,16Z,19Z-docosapentaenoyl)-2-tetradecanoyl-sn-glycero-3-phosphocholine; 1-tetracosanoyl-2-(9Z,12Z-octadecadienoyl)-sn-glycero-3-phosphocholine; 1-(15Z-tetracosenoyl)-2-(9Z-hexadecenoyl)-sn-glycero-3-phosphocholine; 1-(15Z-tetracosenoyl)-2-octadecanoyl-sn-glycero-3-phosphocholine; 1-(15Z-tetracosenoyl)-2-(11Z-octadecenoyl)-sn-glycero-3-phosphocholine; 1-(15Z-tetracosenoyl)-2-(9Z,12Z,15Z-octadecatrienoyl)-sn-glycero-3-phosphocholine; 1-hexadecyl-2-(8Z,11Z,14Z,17Z-eicosatetraenoyl)-sn-glycero-3-phosphocholine; 1-(9Z-hexadecenyl)-2-docosanoyl-sn-glycero-3-phosphocholine; 1-(11Z-octadecenyl)-2-(9Z,12Z-octadecadienoyl)-sn-glycero-3-phosphocholine; 1-(9Z-octadecenyl)-2-(11 Z-octadecenoyl)-sn-glycero-3-phosphocholine; 1-(9Z-octadecenyl)-2-(9Z,12Z-octadecadienoyl)-sn-glycero-3-phosphocholine; 1-(9Z-octadecenyl)-2-eicosanoyl-sn-glycero-3-phosphocholine; 1-(9Z-octadecenyl)-2-(11Z-eicosenoyl)-sn-glycero-3-phosphocholine; 1-(9Z-octadecenyl)-2-(8Z,11Z,14Z,17Z-eicosatetraenoyl)-sn-glycero-3-phosphocholine; 1-(9Z-octadecenyl)-2-docosanoyl-sn-glycero-3-phosphocholine; 1-(9Z-octadecenyl)-2-tetracosanoyl-sn-glycero-3-phosphocholine; 1-(11Z-eicosenyl)-2-(8Z,11Z,14Z,17Z-eicosatetraenoyl)-sn-glycero-3-phosphocholine; 1-docosyl-2-(9Z,12Z,15Z-octadecatrienoyl)-sn-glycero-3-phosphocholine; 1-(13Z-docosenyl)-2-(13Z,16Z-docosadienoyl)-sn-glycero-3-phosphocholine; 1-(13Z-docosenyl)-2-(10Z,13Z,16Z-docosatrienoyl)-sn-glycero-3-phosphocholine; 1-tetracosyl-2-(6Z,9Z,12Z-octadecatrienoyl)-sn-glycero-3-phosphocholine; 1-(1Z-hexadecenyl)-2-(5Z,8Z,11Z-eicosatrienoyl)-sn-glycero-3-phosphocholine; 1-(1Z-octadecenyl)-2-(11Z-octadecenoyl)-sn-glycero-3-phosphocholine; 1-(1Z-octadecenyl)-2-(5Z,8Z,11Z-eicosatrienoyl)-sn-glycero-3-phosphocholine; 1-(1Z,11Z-octadecadienyl)-2-tetradecanoyl-sn-glycero-3-phosphocholine; 1-(1Z,11 Z-octadecadienyl)-2-hexadecanoyl-sn-glycero-3-phosphocholine; 1-(1Z,11Z-octadecadienyl)-2-(9Z-hexadecenoyl)-sn-glycero-3-phosphocholine; 1-(1Z,9Z-octadecadienyl)-2-tetradecanoyl-sn-glycero-3-phosphocholine; 1-(1Z,9Z-octadecadienyl)-2-hexadecanoyl-sn-glycero-3-phosphocholine; 1-(1Z,9Z-octadecadienyl)-2-(13Z-docosenoyl)-sn-glycero-3-phosphocholine; 1-(1Z,9Z-octadecadienyl)-2-tetracosanoyl-sn-glycero-3-phosphocholine; 1-(1Z,9Z-octadecadienyl)-2-(15Z-tetracosenoyl)-sn-glycero-3-phosphocholine; 1-(8Z,11Z,14Z,17Z-eicosatetraenoyl)-sn-glycero-3-phosphocholine; 1-(4Z,7Z,10Z,13Z,16Z-docosapentaenoyl)-sn-glycero-3-phosphocholine; 1-(7Z,10Z,13Z,16Z,19Z-docosapentaenoyl)-sn-glycero-3-phosphocholine; 1-(15Z-tetracosaenoyl)-sn-glycero-3-phosphocholine; 1-(16Z,19Z,22Z,24Z,28Z,31Z-tetratriacontahexaenoyl)-2-(4Z,7Z,10Z,13Z,16Z,19Z-docosahexaenoyl)-glycero-3-phosphocholine; 1-(18Z,21Z,24Z,27Z,30Z-hexatriacontapentaenoyl)-2-(5Z,8Z,11Z,14Z-eicosatetraenoyl)-glycero-3-phosphocholine; 1-(21Z,24Z,27Z,30Z-hexatriacontatetraenoyl)-2-(5Z,8Z,11Z,14Z-eicosatetraenoyl)-glycero-3-phosphocholine; 1-decanoyl-2-dodecanoyl-sn-glycero-3-phosphocholine; 1-decanoyl-2-tetradecanoyl-sn-glycero-3-phosphocholine; 1-undecanoyl-2-tetradecanoyl-sn-glycero-3-phosphocholine; 1-hexadecanoyl-2-decanoyl-sn-glycero-3-phosphocholine; 1-(4Z-octadecenyl-sn-glycero-3-phosphocholine; 1-(13Z-eicosaenoyl)-sn-glycero-3-phosphocholine; 1-(Z)-alk-1-enyl-2-acyl-sn-glycero-3-phosphocholine; 1-(1Z-alkenyl)-sn-glycero-3-phosphocholine; 1-alkyl-2-acyl-sn-glycero-3-phosphocholine; 1-alkyl-sn-glycero-3-phosphocholine; 1-(10Z-heptadecenoyl)-sn-glycero-3-phosphocholine; 1-heptadecanoyl-2-(9Z-tetradecenoyl)-sn-glycero-3-phosphocholine; 1-(10Z,13Z,16Z-nonadecatrienoyl)-sn-glycero-3-phosphocholine; 1-decanoyl-sn-glycero-3-phosphocholine; 1-decanoyl-2-hexadecanoyl-sn-glycero-3-phosphocholine; 1-decanoyl-2-heneicosanoyl-sn-glycero-3-phosphocholine; 1-decanoyl-2-docosanoyl-sn-glycero-3-phosphocholine; 1-decanoyl-2-tricosanoyl-sn-glycero-3-phosphocholine; 1-decanoyl-2-tetracosanoyl-sn-glycero-3-phosphocholine; 1-decanoyl-2-pentacosanoyl-sn-glycero-3-phosphocholine; 1-decanoyl-2-butyryl-sn-glycero-3-phosphocholine; 1-undecanoyl-sn-glycero-3-phosphocholine; 1,2-diundecanoyl-sn-glycero-3-phosphocholine; 1-undecanoyl-2-eicosanoyl-sn-glycero-3-phosphocholine; 1-undecanoyl-2-heneicosanoyl-sn-glycero-3-phosphocholine; 1-undecanoyl-2-tetracosanoyl-sn-glycero-3-phosphocholine; 1-dodecanoyl-2-octadecanoyl-sn-glycero-3-phosphocholine; 1-dodecanoyl-2-(9Z-octadecenoyl)-sn-glycero-3-phosphocholine; 1-dodecanoyl-2-docosanoyl-sn-glycero-3-phosphocholine; 1-dodecanoyl-2-hexacosanoyl-sn-glycero-3-phosphocholine; 1,2-ditridecanoyl-sn-glycero-3-phosphocholine; 1-tridecanoyl-2-nonadecanoyl-sn-glycero-3-phosphocholine; 1-tridecanoyl-2-pentacosanoyl-sn-glycero-3-phosphocholine; 1-tetradecanoyl-sn-glycero-3-phosphocholine; 1-tetradecanoyl-2-octadecanoyl-sn-glycero-3-phosphocholine; 1-tetradecanoyl-2-(11Z,14Z-octadecadienoyl)-sn-glycero-3-phosphocholine; 1-tetradecanoyl-2-(9Z,12Z,15Z-octadecatrienoyl)-sn-glycero-3-phosphocholine; 1-tetradecanoyl-2-(9E,11E,13E,15E-octadecatetraenoyl)-sn-glycero-3-phosphocholine; 1-tetradecanoyl-2-docosanoyl-sn-glycero-3-phosphocholine; 1-tetradecanoyl-2-tetracosanoyl-sn-glycero-3-phosphocholine; 1-tetradecanoyl-2-(15Z-tetracosenoyl)-sn-glycero-3-phosphocholine; 1,2-di-(9E-tetradecenoyl)-sn-glycero-3-phosphocholine; 1-(9Z-tetradecenoyl)-sn-glycero-3-phosphocholine; 1,2-di-(9Z-tetradecenoyl)-sn-glycero-3-phosphocholine; 1-pentadecanoyl-sn-glycero-3-phosphocholine; 1-pentadecanoyl-2-hexadecanoyl-sn-glycero-3-phosphocholine; 1-pentadecanoyl-2-(9Z-hexadecenoyl)-sn-glycero-3-phosphocholine; 1-pentadecanoyl-2-octadecanoyl-sn-glycero-3-phosphocholine; 1-pentadecanoyl-2-(11Z-octadecenoyl)-sn-glycero-3-phosphocholine; 1-pentadecanoyl-2-(9Z,12Z-octadecadienoyl)-sn-glycero-3-phosphocholine; 1-pentadecanoyl-2-heneicosanoyl-sn-glycero-3-phosphocholine; 1-hexadecanoyl-2-dodecanoyl-sn-glycero-3-phosphocholine; 1-hexadecanoyl-2-tetradecanoyl-sn-glycero-3-phosphocholine; 1-hexadecanoyl-2-pentadecanoyl-sn-glycero-3-phosphocholine; 1-hexadecanoyl-2-(9Z-hexadecenoyl)-sn-glycero-3-phosphocholine; 1-hexadecanoyl-2-heptadecanoyl-sn-glycero-3-phosphocholine; 1-hexadecanoyl-2-(9Z-heptadecenoyl)-sn-glycero-3-phosphocholine; 1-hexadecanoyl-2-octadecanoyl-sn-glycero-3-phosphocholine; 1-hexadecanoyl-2-(11E-octadecenoyl)-sn-glycero-3-phosphocholine; 1-hexadecanoyl-2-(11Z,13Z-octadecadienoyl)-sn-glycero-3-phosphocholine; 1-hexadecanoyl-2-(2E,4E-octadecadienoyl)-sn-glycero-3-phosphocholine; 1-hexadecanoyl-2-(2Z,4Z-octadecadienoyl)-sn-glycero-3-phosphocholine; 1-hexadecanoyl-2-(6Z,9Z-octadecadienoyl)-sn-glycero-3-phosphocholine; 1-hexadecanoyl-2-(9E,11E-octadecadienoyl)-sn-glycero-3-phosphocholine; 1-hexadecanoyl-2-(9Z,12Z-octadecadienoyl)-sn-glycero-3-phosphocholine; 1-hexadecanoyl-2-(6Z,9Z,12Z-octadecatrienoyl)-sn-glycero-3-phosphocholine; 1-hexadecanoyl-2-(9Z,12Z,15Z-octadecatrienoyl)-sn-glycero-3-phosphocholine; 1-hexadecanoyl-2-(6Z,9Z,12Z,15Z-octadecatetraenoyl)-sn-glycero-3-phosphocholine; 1-hexadecanoyl-2-(9E,11E,13E,15E-octadecatetraenoyl)-sn-glycero-3-phosphocholine; 1-hexadecanoyl-2-(9Z,11Z,13Z,15Z-octadecatetraenoyl)-sn-glycero-3-phosphocholine; 1-hexadecanoyl-2-acetyl-sn-glycero-3-phosphocholine; 1-hexadecanoyl-2-eicosanoyl-sn-glycero-3-phosphocholine; 1-hexadecanoyl-2-(8E,11E,14E-eicosatrienoyl)-sn-glycero-3-phosphocholine; 1-hexadecanoyl-2-(5E,8E,11E,14E-eicosatetraenoyl)-sn-glycero-3-phosphocholine; 1-hexadecanoyl-2-(13Z-docosenoyl)-sn-glycero-3-phosphocholine; 1-hexadecanoyl-2-(4Z,7Z,10Z,13Z,16Z-docosapentaenoyl)-sn-glycero-3-phosphocholine; 1-hexadecanoyl-2-(7Z,10Z,13Z,16Z,19Z-docosapentaenoyl)-sn-glycero-3-phosphocholine; 1-hexadecanoyl-2-(4E,7E,10E,13E,16E,19E-docosahexaenoyl)-sn-glycero-3-phosphocholine; 1-hexadecanoyl-2-tetracosanoyl-sn-glycero-3-phosphocholine; 1-hexadecanoyl-2-butyryl-sn-glycero-3-phosphocholine; 1-hexadecanoyl-2-valeryl-sn-glycero-3-phosphocholine; 1-hexadecanoyl-2-nonanoyl-sn-glycero-3-phosphocholine; 1-(9E-hexadecenoyl)-sn-glycero-3-phosphocholine; 1-(9Z-hexadecenoyl)-2-(11Z-octadecenoyl)-sn-glycero-3-phosphocholine; 1-(9Z-hexadecenoyl)-2-(9Z-octadecenoyl)-sn-glycero-3-phosphocholine; 1-(9Z-hexadecenoyl)-2-(9Z,12Z-octadecadienoyl)-sn-glycero-3-phosphocholine; 1-(9Z-hexadecenoyl)-2-(6Z,9Z,12Z,15Z-octadecatetraenoyl)-sn-glycero-3-phosphocholine; 1-(9Z-hexadecenoyl)-2-acetyl-sn-glycero-3-phosphocholine; 1-(9Z-hexadecenoyl)-2-(5Z,8Z,11Z,14Z-eicosatetraenoyl)-sn-glycero-3-phosphocholine; 1,2-di-(2E,4E-hexadecadienoyl)-sn-glycero-3-phosphocholine; 1-(2E,4E-hexadecadienoyl)-2-(2E,4E-octadecadienoyl)-sn-glycero-3-phosphocholine; 1,2-diheptadecanoyl-sn-glycero-3-phosphocholine; 1-heptadecanoyl-2-(7Z,10Z,13Z,16Z,19Z-docosapentaenoyl)-sn-glycero-3-phosphocholine; 1-heptadecanoyl-2-(4Z,7Z,10Z,13Z,16Z,19Z-docosahexaenoyl)-sn-glycero-3-phosphocholine; 1,2-di-(10Z-heptadecenoyl)-sn-glycero-3-phosphocholine; 1,2-di-(9Z-heptadecenoyl)-sn-glycero-3-phosphocholine; 1-octadecanoyl-sn-glycero-3-phosphocholine; 1-octadecanoyl-2-decanoyl-sn-glycero-3-phosphocholine; 1-octadecanoyl-2-(10E-undecenoyl)-sn-glycero-3-phosphocholine; 1-octadecanoyl-2-dodecanoyl-sn-glycero-3-phosphocholine; 1-octadecanoyl-2-hexadecanoyl-sn-glycero-3-phosphocholine; 1-octadecanoyl-2-(2E,4E-hexadecadienoyl)-sn-glycero-3-phosphocholine; 1-octadecanoyl-2-(12Z-octadecenoyl)-sn-glycero-3-phosphocholine; 1-octadecanoyl-2-(16Z-octadecenoyl)-sn-glycero-3-phosphocholine; 1-octadecanoyl-2-(6Z-octadecenoyl)-sn-glycero-3-phosphocholine; 1-octadecanoyl-2-(7Z-octadecenoyl)-sn-glycero-3-phosphocholine; 1-octadecanoyl-2-(9Z-octadecenoyl)-sn-glycero-3-phosphocholine; 1-octadecanoyl-2-(10Z,12Z-octadecadienoyl)-sn-glycero-3-phosphocholine; 1-octadecanoyl-2-(2E,4E-octadecadienoyl)-sn-glycero-3-phosphocholine; 1-octadecanoyl-2-(6Z,9Z,12Z,15Z-octadecatetraenoyl)-sn-glycero-3-phosphocholine; 1-octadecanoyl-2-(11Z-eicosenoyl)-sn-glycero-3-phosphocholine; 1-octadecanoyl-2-(8Z-eicosenoyl)-sn-glycero-3-phosphocholine; 1-octadecanoyl-2-(11Z,14Z-eicosadienoyl)-sn-glycero-3-phosphocholine; 1-octadecanoyl-2-(5Z,14Z-eicosadienoyl)-sn-glycero-3-phosphocholine; 1-octadecanoyl-2-(8Z,14Z-eicosadienoyl)-sn-glycero-3-phosphocholine; 1-octadecanoyl-2-(5Z,11Z,14Z-eicosatrienoyl)-sn-glycero-3-phosphocholine; 1-octadecanoyl-2-(5Z,8Z,11Z-eicosatrienoyl)-sn-glycero-3-phosphocholine; 1-octadecanoyl-2-(8Z,11Z,14Z-eicosatrienoyl)-sn-glycero-3-phosphocholine; 1-octadecanoyl-2-docosanoyl-sn-glycero-3-phosphocholine; 1-octadecanoyl-2-(13Z-docosenoyl)-sn-glycero-3-phosphocholine; 1-octadecanoyl-2-(10Z,13Z,16Z-docosatrienoyl)-sn-glycero-3-phosphocholine; 1-octadecanoyl-2-(7Z,10Z,13Z,16Z-docosatetraenoyl)-sn-glycero-3-phosphocholine; 1-octadecanoyl-2-(4Z,7Z,10Z,13Z,16Z-docosapentaenoyl)-sn-glycero-3-phosphocholine; 1-octadecanoyl-2-(4Z,7Z,10Z,13Z,16Z,19Z-docosahexaenoyl)-sn-glycero-3-phosphocholine; 1-octadecanoyl-2-tetracosanoyl-sn-glycero-3-phosphocholine; 1-octadecanoyl-2-(15Z-tetracosenoyl)-sn-glycero-3-phosphocholine; 1,2-di-(10Z-octadecenoyl)-sn-glycero-3-phosphocholine; 1-(11Z-octadecenoyl)-2-(13Z-eicosenoyl)-sn-glycero-3-phosphocholine; 1-(11Z-octadecenoyl)-2-(5Z,8Z,11Z,14Z,17Z-eicosapentaenoyl)-sn-glycero-3-phosphocholine; 1-(11Z-octadecenoyl)-2-(4Z,7Z,10Z,13Z,16Z,19Z-docosahexaenoyl)-sn-glycero-3-phosphocholine; 1,2-di-(12Z-octadecenoyl)-sn-glycero-3-phosphocholine; 1,2-di-(15Z-octadecenoyl)-sn-glycero-3-phosphocholine; 1,2-di-(17Z-octadecenoyl)-sn-glycero-3-phosphocholine; 1,2-di-(3Z-octadecenoyl)-sn-glycero-3-phosphocholine; 1,2-di-(6E-octadecenoyl)-sn-glycero-3-phosphocholine; 1-(6Z-octadecenoyl)-sn-glycero-3-phosphocholine; 1,2-di-(6Z-octadecenoyl)-sn-glycero-3-phosphocholine; 1,2-di-(7Z-octadecenoyl)-sn-glycero-3-phosphocholine; 1,2-di-(9E-octadecenoyl)-sn-glycero-3-phosphocholine; 1-(9E-octadecenoyl)-2-acetyl-sn-glycero-3-phosphocholine; 1-(9E-octadecenoyl)-2-eicosanoyl-sn-glycero-3-phosphocholine; 1-(9Z-octadecenoyl)-2-hexadecanoyl-sn-glycero-3-phosphocholine; 1-(9Z-octadecenoyl)-2-(9Z-hexadecenoyl)-sn-glycero-3-phosphocholine; 1-(9Z-octadecenoyl)-2-(9Z,12Z-octadecadienoyl)-sn-glycero-3-phosphocholine; 1-(9Z-octadecenoyl)-2-eicosanoyl-sn-glycero-3-phosphocholine; 1-(9Z-octadecenoyl)-2-(5Z,8Z,11Z,14Z-eicosatetraenoyl)-sn-glycero-3-phosphocholine; 1-(9Z-octadecenoyl)-2-(5Z,8Z,11Z,14Z,17Z-eicosapentaenoyl)-sn-glycero-3-phosphocholine; 1-(2E,4E-octadecadienoyl)-sn-glycero-3-phosphocholine; 1,2-di-(2E,4E-octadecadienoyl)-sn-glycero-3-phosphocholine; 1,2-di-(2Z,4Z-octadecadienoyl)-sn-glycero-3-phosphocholine; 1-(9Z,12Z-octadecadienoyl)-sn-glycero-3-phosphocholine; 1-(9Z,12Z-octadecadienoyl)-2-octadecanoyl-sn-glycero-3-phosphocholine; 1,2-di-(9Z,12Z-octadecadienoyl)-sn-glycero-3-phosphocholine; 1-(9Z,12Z-octadecadienoyl)-2-(4Z,7Z,10Z,13Z,16Z-docosapentaenoyl)-sn-glycero-3-phosphocholine; 1,2-di-(9Z,11E,13E-octadecatrienoyl)-sn-glycero-3-phosphocholine; 1,2-di-(9Z,12Z,15Z-octadecatrienoyl)-sn-glycero-3-phosphocholine; 1-(9E,11E,13E,15E-octadecatetraenoyl)-sn-glycero-3-phosphocholine; 1-(9E,11E,13E,15E-octadecatetraenoyl)-2-(9Z,12Z-octadecadienoyl)-sn-glycero-3-phosphocholine; 1-(9Z,11E,13E,15Z-octadecatetraenoyl)-2-hexadecanoyl-sn-glycero-3-phosphocholine; 1-nonadecanoyl-2-dodecanoyl-sn-glycero-3-phosphocholine; 1-nonadecanoyl-sn-glycero-3-phosphocholine; 1-nonadecanoyl-2-decanoyl-sn-glycero-3-phosphocholine; 1-nonadecanoyl-2-octadecanoyl-sn-glycero-3-phosphocholine; 1,2-dinonadecanoyl-sn-glycero-3-phosphocholine; 1-nonadecanoyl-2-acetyl-sn-glycero-3-phosphocholine; 1-nonadecanoyl-2-pentacosanoyl-sn-glycero-3-phosphocholine; 1-acetyl-sn-glycero-3-phosphocholine; 1-acetyl-2-hexadecanoyl-sn-glycero-3-phosphocholine; 1-acetyl-2-octadecanoyl-sn-glycero-3-phosphocholine; 1-acetyl-2-(9Z-octadecenoyl)-sn-glycero-3-phosphocholine; 1,2-diacetyl-sn-glycero-3-phosphocholine; 1-eicosanoyl-sn-glycero-3-phosphocholine; 1-eicosanoyl-2-decanoyl-sn-glycero-3-phosphocholine; 1-eicosanoyl-2-undecanoyl-sn-glycero-3-phosphocholine; 1-eicosanoyl-2-tridecanoyl-sn-glycero-3-phosphocholine; 1-eicosanoyl-2-hexadecanoyl-sn-glycero-3-phosphocholine; 1-eicosanoyl-2-octadecanoyl-sn-glycero-3-phosphocholine; 1,2-dieicosanoyl-sn-glycero-3-phosphocholine; 1-eicosanoyl-2-(11Z-eicosenoyl)-sn-glycero-3-phosphocholine; 1-eicosanoyl-2-(13Z-eicosenoyl)-sn-glycero-3-phosphocholine; 1-eicosanoyl-2-(5Z-eicosenoyl)-sn-glycero-3-phosphocholine; 1-eicosanoyl-2-(11Z,14Z-eicosadienoyl)-sn-glycero-3-phosphocholine; 1-eicosanoyl-2-(5Z,8Z,11Z,14Z-eicosatetraenoyl)-sn-glycero-3-phosphocholine; 1-eicosanoyl-2-(7Z,10Z,13Z,16Z,19Z-docosapentaenoyl)-sn-glycero-3-phosphocholine; 1-eicosanoyl-2-(15Z-tetracosenoyl)-sn-glycero-3-phosphocholine; 1-(13Z-eicosenoyl)-2-docosanoyl-sn-glycero-3-phosphocholine; 1,2-di-(9E-eicosenoyl)-sn-glycero-3-phosphocholine; 1,2-di-(9Z-eicosenoyl)-sn-glycero-3-phosphocholine; 1,2-di-(11E,14E-eicosadienoyl)-sn-glycero-3-phosphocholine; 1,2-di-(5E,8E,11E,14E-eicosatetraenoyl)-sn-glycero-3-phosphocholine; 1-(5Z,8Z,11Z,14Z-eicosatetraenoyl)-sn-glycero-3-phosphocholine; 1,2-di-(5Z,8Z,11Z,14Z-eicosatetraenoyl)-sn-glycero-3-phosphocholine; 1-(5Z,8Z,11Z,14Z,17Z-eicosapentaenoyl)-sn-glycero-3-phosphocholine; 1-heneicosanoyl-sn-glycero-3-phosphocholine; 1-heneicosanoyl-2-undecanoyl-sn-glycero-3-phosphocholine; 1-heneicosanoyl-2-tetradecanoyl-sn-glycero-3-phosphocholine; 1-docosanoyl-2-dodecanoyl-sn-glycero-3-phosphocholine; 1-docosanoyl-2-tridecanoyl-sn-glycero-3-phosphocholine; 1-docosanoyl-2-tetradecanoyl-sn-glycero-3-phosphocholine; 1-docosanoyl-2-(13Z-octadecenoyl)-sn-glycero-3-phosphocholine; 1-docosanoyl-2-(6Z-octadecenoyl)-sn-glycero-3-phosphocholine; 1-docosanoyl-2-(7Z-octadecenoyl)-sn-glycero-3-phosphocholine; 1-docosanoyl-2-(9Z-octadecenoyl)-sn-glycero-3-phosphocholine; 1-docosanoyl-2-(11Z-eicosenoyl)-sn-glycero-3-phosphocholine; 1-docosanoyl-2-(15Z-tetracosenoyl)-sn-glycero-3-phosphocholine; 1,2-di-(13E-docosenoyl)-sn-glycero-3-phosphocholine; 1,2-di-(13Z-docosenoyl)-sn-glycero-3-phosphocholine; 1-(4E,7E,10E,13E,16E,19E-docosahexaenoyl)-2-hexadecanoyl-sn-glycero-3-phosphocholine; 1,2-di-(4Z,7Z,10Z,13Z,16Z,19Z-docosahexaenoyl)-sn-glycero-3-phosphocholine; 1,2-di-(9Z-tricosenoyl)-sn-glycero-3-phosphocholine; 1-tetracosanoyl-sn-glycero-3-phosphocholine; 1-tetracosanoyl-2-hexadecanoyl-sn-glycero-3-phosphocholine; 1-tetracosanoyl-2-(11Z-eicosenoyl)-sn-glycero-3-phosphocholine; 1,2-di-(5E,9E-hexacosadienoyl)-sn-glycero-3-phosphocholine; 1,2-di-(5E,9Z-hexacosadienoyl)-sn-glycero-3-phosphocholine; 1,2-di-(5Z,9E-hexacosadienoyl)-sn-glycero-3-phosphocholine; 1-(5Z,9Z-hexacosadienoyl)-2-hexadecanoyl-sn-glycero-3-phosphocholine; 1,2-di-(5Z,9Z-hexacosadienoyl)-sn-glycero-3-phosphocholine; 1,2-di-(6Z,9Z-hexacosadienoyl)-sn-glycero-3-phosphocholine; 1-butyryl-sn-glycero-3-phosphocholine; 1-butyryl-2-(9Z-octadecenoyl)-sn-glycero-3-phosphocholine; 1,2-divaleryl-sn-glycero-3-phosphocholine; 1,2-dihexanoyl-sn-glycero-3-phosphocholine; 1-hexanoyl-2-octanoyl-sn-glycero-3-phosphocholine; 1,2-di-(2E,4E-hexadienoyl)-sn-glycero-3-phosphocholine; 1-(3E,5E-hexadienoyl)-2-(11E,13E-tetradecadienoyl)-sn-glycero-3-phosphocholine; 1-heptanoyl-sn-glycero-3-phosphocholine; 1-heptanoyl-2-heneicosanoyl-sn-glycero-3-phosphocholine; 1,2-diheptanoyl-sn-glycero-3-phosphocholine; 1-octanoyl-sn-glycero-3-phosphocholine; 1-octanoyl-2-nonadecanoyl-sn-glycero-3-phosphocholine; 1-octanoyl-2-heneicosanoyl-sn-glycero-3-phosphocholine; 1-octanoyl-2-hexanoyl-sn-glycero-3-phosphocholine; 1,2-di-(2E,4E-octadienoyl)-sn-glycero-3-phosphocholine; 1-nonanoyl-sn-glycero-3-phosphocholine; 1-nonanoyl-2-docosanoyl-sn-glycero-3-phosphocholine; 1-nonanoyl-2-tricosanoyl-sn-glycero-3-phosphocholine; 1-nonanoyl-2-valeryl-sn-glycero-3-phosphocholine; 1-methyl-2-hexadecanoyl-sn-glycero-3-phosphocholine; 1-methyl-2-acetyl-sn-glycero-3-phosphocholine; 1-dodecyl-2-acetyl-sn-glycero-3-phosphocholine; 1-tetradecyl-2-pentadecanoyl-sn-glycero-3-phosphocholine; 1-tetradecyl-2-(9Z-hexadecenoyl)-sn-glycero-3-phosphocholine; 1-tetradecyl-2-octadecanoyl-sn-glycero-3-phosphocholine; 1-tetradecyl-2-(9Z-octadecenoyl)-sn-glycero-3-phosphocholine; 1-tetradecyl-2-(9Z,12Z-octadecadienoyl)-sn-glycero-3-phosphocholine; 1-tetradecyl-2-(9Z,12Z,15Z-octadecatrienoyl)-sn-glycero-3-phosphocholine; 1-tetradecyl-2-docosanoyl-sn-glycero-3-phosphocholine; 1-pentadecyl-2-acetyl-sn-glycero-3-phosphocholine; 1-hexadecyl-2-formyl-sn-glycero-3-phosphocholine; 1-hexadecyl-2-hexadecanoyl-sn-glycero-3-phosphocholine; 1-hexadecyl-2-heptadecanoyl-sn-glycero-3-phosphocholine; 1-hexadecyl-2-octadecanoyl-sn-glycero-3-phosphocholine; 1-hexadecyl-2-nonadecanoyl-sn-glycero-3-phosphocholine; 1-hexadecyl-2-(9Z-eicosenoyl)-sn-glycero-3-phosphocholine; 1-hexadecyl-2-(8Z,11Z,14Z-eicosatrienoyl)-sn-glycero-3-phosphocholine; 1-hexadecyl-2-(5Z,8Z,11Z,14Z-eicosatetraenoyl)-sn-glycero-3-phosphocholine; 1-hexadecyl-2-docosanoyl-sn-glycero-3-phosphocholine; 1-hexadecyl-2-(4Z,7Z,10Z,13Z,16Z,19Z-docosahexaenoyl)-sn-glycero-3-phosphocholine; 1-hexadecyl-2-butyryl-sn-glycero-3-phosphocholine; 1-hexadecyl-2-valeryl-sn-glycero-3-phosphocholine; 1-(1Z-hexadecenyl)-2-(9Z-octadecenoyl)-sn-glycero-3-phosphocholine; 1-(1Z-hexadecenyl)-2-acetyl-sn-glycero-3-phosphocholine; 1-(1Z-hexadecenyl)-2-(5Z,8Z,11Z,14Z-eicosatetraenoyl)-sn-glycero-3-phosphocholine; 1-heptadecyl-2-(9Z-octadecenoyl)-sn-glycero-3-phosphocholine; 1-octadecyl-2-hexadecanoyl-sn-glycero-3-phosphocholine; 1-octadecyl-2-(9Z-hexadecenoyl)-sn-glycero-3-phosphocholine; 1-octadecyl-2-octadecanoyl-sn-glycero-3-phosphocholine; 1-octadecyl-2-acetyl-sn-glycero-3-phosphocholine; 1-octadecyl-2-eicosanoyl-sn-glycero-3-phosphocholine; 1-octadecyl-2-(5E,8E,11E,14E-eicosatetraenoyl)-sn-glycero-3-phosphocholine; 1-octadecyl-2-(5Z,8Z,11Z,14Z-eicosatetraenoyl)-sn-glycero-3-phosphocholine; 1-octadecyl-2-(5Z,8Z,11Z,14Z,17Z-eicosapentaenoyl)-sn-glycero-3-phosphocholine; 1-octadecyl-2-(4Z,7Z,10Z,13Z,16Z-docosapentaenoyl)-sn-glycero-3-phosphocholine; 1-octadecyl-2-(7Z,10Z,13Z,16Z,19Z-docosapentaenoyl)-sn-glycero-3-phosphocholine; 1-octadecyl-2-propionyl-sn-glycero-3-phosphocholine; 1-octadecyl-2-(2E-propionyl)-sn-glycero-3-phosphocholine; 1-octadecyl-2-butyryl-sn-glycero-3-phosphocholine; 1-(1Z-octadecenyl)-2-(5Z,8Z,11Z,14Z-eicosatetraenoyl)-sn-glycero-3-phosphocholine; 1-nonadecyl-2-acetyl-sn-glycero-3-phosphocholine; 1-ethyl-2-acetyl-sn-glycero-3-phosphocholine; 1-eicosyl-2-hexadecanoyl-sn-glycero-3-phosphocholine; 1-eicosyl-2-acetyl-sn-glycero-3-phosphocholine; 1-eicosyl-2-docosanoyl-sn-glycero-3-phosphocholine; 1-octyl-2-acetyl-sn-glycero-3-phosphocholine; 2-hexadecanoyl-sn-glycero-3-phosphocholine; 2-(6Z-octadecenoyl)-sn-glycero-3-phosphocholine; 2-acetyl-sn-glycero-3-phosphocholine; 2-propionyl-sn-glycero-3-phosphocholine; 1-tetradecyl-sn-glycero-3-phosphocholine; 1-(1Z-pentadecenyl)-sn-glycero-3-phosphocholine; 1-(11Z-hexadecenyl)-sn-glycero-3-phosphocholine; 1-(9E-hexadecenyl)-sn-glycero-3-phosphocholine; 1-(9Z-hexadecenyl)-sn-glycero-3-phosphocholine; 1-heptadecyl-sn-glycero-3-phosphocholine; 1-(1Z-heptadecenyl)-sn-glycero-3-phosphocholine; 1-octadecyl-sn-glycero-3-phosphocholine; 1-(1Z,9Z-octadecadienyl)-sn-glycero-3-phosphocholine; 1-hexadecanoyl-sn-glycero-3-phosphocholine; 1-(7Z-hexadecenoyl)-2-(5Z,8Z,11 Z,14Z-eicosatetraenoyl)-sn-glycero-3-phosphocholine; 1,2-diacyl-sn-glycero-3-phosphocholine; 2-acyl-sn-glycero-3-phosphocholine; 2-(8-[3]-ladderane-octanyl)-sn-glycero-3-phosphocholine; 1-(6-[3]-ladderane-hexanyl)-2-(8-[3]-ladderane-octanyl)-sn-glycerophosphocholine; 1-(10-methylhexadecanyl)-2-(8-[3]-ladderane-octanyl)-sn-glycerophosphocholine; 1-tetradecanyl-2-(8-[3]-ladderane-octanyl)-sn-glycerophosphocholine; 1-(10E-undecenyl)-2-acetyl-sn-glycero-3-phosphocholine; 1-(11Z-hexadecenyl)-2-acetyl-sn-glycero-3-phosphocholine; 1-(9Z-octadecenyl)-2-hexadecanoyl-sn-glycero-3-phosphocholine; 1-(9Z-octadecenyl)-2-acetyl-sn-glycero-3-phosphocholine; 1-(2E,6E,10E,14E-phytatetraenyl)-2-(2E,6E,10E,14E-phytatetraenyl)-sn-glycero-3-phosphocholine; 1,2-diphytanyl-sn-glycero-3-phosphocholine; 1-(1Z-octadecenyl)-2-(4Z,7Z,10Z,13Z,16Z,19Z-docosahexaenoyl)-sn-glycero-3-phosphocholine; 1-(1Z,12Z-nonadecadienyl)-sn-glycero-3-phosphocholine; 2-(5Z,8Z,11Z,14Z-eicosatetraenoyl)-sn-glycero-3-phosphocholine; 1-dodecanoyl-2-(9Z,12Z-heptadecadienoyl)-glycero-3-phosphocholine; 1-dodecanoyl-2-(9Z,12Z-octadecadienoyl)-glycero-3-phosphocholine; 1-dodecanoyl-2-(6Z,9Z,12Z-octadecatrienoyl)-glycero-3-phosphocholine; 1-dodecanoyl-2-(9Z,12Z,15Z-octadecatrienoyl)-glycero-3-phosphocholine; 1-dodecanoyl-2-(6Z,9Z,12Z,15Z-octadecatetraenoyl)-glycero-3-phosphocholine; 1-dodecanoyl-2-(11Z-eicosenoyl)-glycero-3-phosphocholine; 1-dodecanoyl-2-(11Z,14Z-eicosadienoyl)-glycero-3-phosphocholine; 1-dodecanoyl-2-(8Z,11Z,14Z-eicosatrienoyl)-glycero-3-phosphocholine; 1-dodecanoyl-2-(11Z-docosenoyl)-glycero-3-phosphocholine; 1-dodecanoyl-2-(13Z,16Z-docosadienoyl)-glycero-3-phosphocholine; 1-dodecanoyl-2-(7Z,10Z,13Z,16Z-docosatetraenoyl)-glycero-3-phosphocholine; 1-tridecanoyl-2-dodecanoyl-glycero-3-phosphocholine; 1-tridecanoyl-2-tetradecanoyl-glycero-3-phosphocholine; 1-tridecanoyl-2-(9Z-tetradecenoyl)-glycero-3-phosphocholine; 1-tridecanoyl-2-(9Z-pentadecenoyl)-glycero-3-phosphocholine; 1-tridecanoyl-2-hexadecanoyl-glycero-3-phosphocholine; 1-tridecanoyl-2-(9Z-heptadecenoyl)-glycero-3-phosphocholine; 1-tridecanoyl-2-octadecanoyl-glycero-3-phosphocholine; 1-tridecanoyl-2-(9Z-octadecenoyl)-glycero-3-phosphocholine; 1-tridecanoyl-2-(9Z,12Z-octadecadienoyl)-glycero-3-phosphocholine; 1-tridecanoyl-2-(9Z,12Z,15Z-octadecatrienoyl)-glycero-3-phosphocholine; 1-tridecanoyl-2-(6Z,9Z,12Z,15Z-octadecatetraenoyl)-glycero-3-phosphocholine; 1-tridecanoyl-2-(11Z,14Z-eicosadienoyl)-glycero-3-phosphocholine; 1-tridecanoyl-2-(5Z,8Z,11Z,14Z-eicosatetraenoyl)-glycero-3-phosphocholine; 1-tridecanoyl-2-docosanoyl-glycero-3-phosphocholine; 1-tridecanoyl-2-(7Z,10Z,13Z,16Z-docosatetraenoyl)-glycero-3-phosphocholine; 1-tridecanoyl-2-(4Z,7Z,10Z,13Z,16Z,19Z-docosahexaenoyl)-glycero-3-phosphocholine; 1-tetradecanoyl-2-(9Z-tetradecenoyl)-glycero-3-phosphocholine; 1-tetradecanoyl-2-heptadecanoyl-glycero-3-phosphocholine; 1-tetradecanoyl-2-(9Z,12Z-heptadecadienoyl)-glycero-3-phosphocholine; 1-tetradecanoyl-2-(6Z,9Z,12Z-octadecatrienoyl)-glycero-3-phosphocholine; 1-tetradecanoyl-2-(9Z-nonadecenoyl)-glycero-3-phosphocholine; 1-tetradecanoyl-2-(8Z,11Z,14Z-eicosatrienoyl)-glycero-3-phosphocholine; 1-tetradecanoyl-2-heneicosanoyl-glycero-3-phosphocholine; 1-tetradecanoyl-2-(7Z,10Z,13Z,16Z-docosatetraenoyl)-glycero-3-phosphocholine; 1-(9Z-tetradecenoyl)-2-tridecanoyl-glycero-3-phosphocholine; 1-(9Z-tetradecenoyl)-2-pentadecanoyl-glycero-3-phosphocholine; 1-(9Z-tetradecenoyl)-2-(9Z-octadecenoyl)-glycero-3-phosphocholine; 1-(9Z-tetradecenoyl)-2-(9Z,12Z-octadecadienoyl)-glycero-3-phosphocholine; 1-(9Z-tetradecenoyl)-2-(6Z,9Z,12Z-octadecatrienoyl)-glycero-3-phosphocholine; 1-(9Z-tetradecenoyl)-2-nonadecanoyl-glycero-3-phosphocholine; 1-(9Z-tetradecenoyl)-2-(9Z-nonadecenoyl)-glycero-3-phosphocholine; 1-(9Z-tetradecenoyl)-2-(8Z,11Z,14Z-eicosatrienoyl)-glycero-3-phosphocholine; 1-(9Z-tetradecenoyl)-2-(5Z,8Z,11Z,14Z,17Z-eicosapentaenoyl)-glycero-3-phosphocholine; 1-(9Z-tetradecenoyl)-2-heneicosanoyl-glycero-3-phosphocholine; 1-(9Z-tetradecenoyl)-2-docosanoyl-glycero-3-phosphocholine; 1-(9Z-tetradecenoyl)-2-(11Z-docosenoyl)-glycero-3-phosphocholine; 1-(9Z-tetradecenoyl)-2-(7Z,10Z,13Z,16Z-docosatetraenoyl)-glycero-3-phosphocholine; 1-pentadecanoyl-2-dodecanoyl-glycero-3-phosphocholine; 1-pentadecanoyl-2-(9Z-pentadecenoyl)-glycero-3-phosphocholine; 1-pentadecanoyl-2-(9Z,12Z-heptadecadienoyl)-glycero-3-phosphocholine; 1-pentadecanoyl-2-eicosanoyl-glycero-3-phosphocholine; 1-pentadecanoyl-2-(5Z,8Z,11Z,14Z-eicosatetraenoyl)-glycero-3-phosphocholine; 1-pentadecanoyl-2-(13Z,16Z-docosadienoyl)-glycero-3-phosphocholine; 1-pentadecanoyl-2-(7Z,10Z,13Z,16Z-docosatetraenoyl)-glycero-3-phosphocholine; 1-(9Z-pentadecenoyl)-glycero-3-phosphocholine; 1-(9Z-pentadecenoyl)-2-dodecanoyl-glycero-3-phosphocholine; 1-(9Z-pentadecenoyl)-2-tetradecanoyl-glycero-3-phosphocholine; 1-(9Z-pentadecenoyl)-2-heptadecanoyl-glycero-3-phosphocholine; 1-(9Z-pentadecenoyl)-2-(9Z,12Z-heptadecadienoyl)-glycero-3-phosphocholine; 1-(9Z-pentadecenoyl)-2-(9Z-octadecenoyl)-glycero-3-phosphocholine; 1-(9Z-pentadecenoyl)-2-(6Z,9Z,12Z-octadecatrienoyl)-glycero-3-phosphocholine; 1-(9Z-pentadecenoyl)-2-(6Z,9Z,12Z,15Z-octadecatetraenoyl)-glycero-3-phosphocholine; 1-(9Z-pentadecenoyl)-2-nonadecanoyl-glycero-3-phosphocholine; 1-(9Z-pentadecenoyl)-2-(9Z-nonadecenoyl)-glycero-3-phosphocholine; 1-(9Z-pentadecenoyl)-2-(11Z-eicosenoyl)-glycero-3-phosphocholine; 1-(9Z-pentadecenoyl)-2-docosanoyl-glycero-3-phosphocholine; 1-(9Z-pentadecenoyl)-2-(11Z-docosenoyl)-glycero-3-phosphocholine; 1-(9Z-pentadecenoyl)-2-(7Z,10Z,13Z,16Z-docosatetraenoyl)-glycero-3-phosphocholine; 1-(9Z-pentadecenoyl)-2-(4Z,7Z,10Z,13Z,16Z,19Z-docosahexaenoyl)-glycero-3-phosphocholine; 1-hexadecanoyl-2-(9Z-pentadecenoyl)-glycero-3-phosphocholine; 1-hexadecanoyl-2-(9Z,12Z-heptadecadienoyl)-glycero-3-phosphocholine; 1-hexadecanoyl-2-nonadecanoyl-glycero-3-phosphocholine; 1-hexadecanoyl-2-(11Z-eicosenoyl)-glycero-3-phosphocholine; 1-hexadecanoyl-2-heneicosanoyl-glycero-3-phosphocholine; 1-(9Z-hexadecenoyl)-2-tridecanoyl-glycero-3-phosphocholine; 1-(9Z-hexadecenoyl)-2-(9Z-tetradecenoyl)-glycero-3-phosphocholine; 1-(9Z-hexadecenoyl)-2-hexadecanoyl-glycero-3-phosphocholine; 1-(9Z-hexadecenoyl)-2-(9Z-heptadecenoyl)-glycero-3-phosphocholine; 1-(9Z-hexadecenoyl)-2-octadecanoyl-glycero-3-phosphocholine; 1-(9Z-hexadecenoyl)-2-(9Z,12Z,15Z-octadecatrienoyl)-glycero-3-phosphocholine; 1-(9Z-hexadecenoyl)-2-(9Z-nonadecenoyl)-glycero-3-phosphocholine; 1-(9Z-hexadecenoyl)-2-(8Z,11Z,14Z-eicosatrienoyl)-glycero-3-phosphocholine; 1-(9Z-hexadecenoyl)-2-(5Z,8Z,11Z,14Z,17Z-eicosapentaenoyl)-glycero-3-phosphocholine; 1-(9Z-hexadecenoyl)-2-heneicosanoyl-glycero-3-phosphocholine; 1-(9Z-hexadecenoyl)-2-(11Z-docosenoyl)-glycero-3-phosphocholine; 1-(9Z-hexadecenoyl)-2-(7Z,10Z,13Z,16Z-docosatetraenoyl)-glycero-3-phosphocholine; 1-heptadecanoyl-2-dodecanoyl-glycero-3-phosphocholine; 1-heptadecanoyl-2-tetradecanoyl-glycero-3-phosphocholine; 1-heptadecanoyl-2-(9Z-pentadecenoyl)-glycero-3-phosphocholine; 1-heptadecanoyl-2-hexadecanoyl-glycero-3-phosphocholine; 1-heptadecanoyl-2-(9Z-hexadecenoyl)-glycero-3-phosphocholine; 1-heptadecanoyl-2-(9Z-heptadecenoyl)-glycero-3-phosphocholine; 1-heptadecanoyl-2-(9Z,12Z-heptadecadienoyl)-glycero-3-phosphocholine; 1-heptadecanoyl-2-(9Z,12Z-octadecadienoyl)-glycero-3-phosphocholine; 1-heptadecanoyl-2-(9Z,12Z,15Z-octadecatrienoyl)-glycero-3-phosphocholine; 1-heptadecanoyl-2-eicosanoyl-glycero-3-phosphocholine; 1-heptadecanoyl-2-(11Z-eicosenoyl)-glycero-3-phosphocholine; 1-heptadecanoyl-2-(8Z,11Z,14Z-eicosatrienoyl)-glycero-3-phosphocholine; 1-heptadecanoyl-2-(5Z,8Z,11Z,14Z,17Z-eicosapentaenoyl)-glycero-3-phosphocholine; 1-heptadecanoyl-2-(7Z,10Z,13Z,16Z-docosatetraenoyl)-glycero-3-phosphocholine; 1-(9Z-heptadecenoyl)-glycero-3-phosphocholine; 1-(9Z-heptadecenoyl)-2-dodecanoyl-glycero-3-phosphocholine; 1-(9Z-heptadecenoyl)-2-tridecanoyl-glycero-3-phosphocholine; 1-(9Z-heptadecenoyl)-2-tetradecanoyl-glycero-3-phosphocholine; 1-(9Z-heptadecenoyl)-2-(9Z-tetradecenoyl)-glycero-3-phosphocholine; 1-(9Z-heptadecenoyl)-2-pentadecanoyl-glycero-3-phosphocholine; 1-(9Z-heptadecenoyl)-2-(9Z-pentadecenoyl)-glycero-3-phosphocholine; 1-(9Z-heptadecenoyl)-2-octadecanoyl-glycero-3-phosphocholine; 1-(9Z-heptadecenoyl)-2-(9Z-octadecenoyl)-glycero-3-phosphocholine; 1-(9Z-heptadecenoyl)-2-(9Z,12Z-octadecadienoyl)-glycero-3-phosphocholine; 1-(9Z-heptadecenoyl)-2-(9Z,12Z,15Z-octadecatrienoyl)-glycero-3-phosphocholine; 1-(9Z-heptadecenoyl)-2-nonadecanoyl-glycero-3-phosphocholine; 1-(9Z-heptadecenoyl)-2-(9Z-nonadecenoyl)-glycero-3-phosphocholine; 1-(9Z-heptadecenoyl)-2-eicosanoyl-glycero-3-phosphocholine; 1-(9Z-heptadecenoyl)-2-(11Z,14Z-eicosadienoyl)-glycero-3-phosphocholine; 1-(9Z-heptadecenoyl)-2-(8Z,11Z,14Z-eicosatrienoyl)-glycero-3-phosphocholine; 1-(9Z-heptadecenoyl)-2-(5Z,8Z,11Z,14Z-eicosatetraenoyl)-glycero-3-phosphocholine; 1-(9Z-heptadecenoyl)-2-(5Z,8Z,11Z,14Z,17Z-eicosapentaenoyl)-glycero-3-phosphocholine; 1-(9Z-heptadecenoyl)-2-(11Z-docosenoyl)-glycero-3-phosphocholine; 1-(9Z-heptadecenoyl)-2-(13Z,16Z-docosadienoyl)-glycero-3-phosphocholine; 1-(9Z-heptadecenoyl)-2-(4Z,7Z,10Z,13Z,16Z,19Z-docosahexaenoyl)-glycero-3-phosphocholine; 1-(9Z,12Z-heptadecadienoyl)-glycero-3-phosphocholine; 1-(9Z,12Z-heptadecadienoyl)-2-pentadecanoyl-glycero-3-phosphocholine; 1-(9Z,12Z-heptadecadienoyl)-2-hexadecanoyl-glycero-3-phosphocholine; 1-(9Z,12Z-heptadecadienoyl)-2-(9Z-hexadecenoyl)-glycero-3-phosphocholine; 1-(9Z,12Z-heptadecadienoyl)-2-(9Z-heptadecenoyl)-glycero-3-phosphocholine; 1-(9Z,12Z-heptadecadienoyl)-2-nonadecanoyl-glycero-3-phosphocholine; 1-(9Z,12Z-heptadecadienoyl)-2-(9Z-nonadecenoyl)-glycero-3-phosphocholine; 1-(9Z,12Z-heptadecadienoyl)-2-eicosanoyl-glycero-3-phosphocholine; 1-(9Z,12Z-heptadecadienoyl)-2-(8Z,11Z,14Z-eicosatrienoyl)-glycero-3-phosphocholine; 1-(9Z,12Z-heptadecadienoyl)-2-(5Z,8Z,11Z,14Z,17Z-eicosapentaenoyl)-glycero-3-phosphocholine; 1-(9Z,12Z-heptadecadienoyl)-2-heneicosanoyl-glycero-3-phosphocholine; 1-(9Z,12Z-heptadecadienoyl)-2-docosanoyl-glycero-3-phosphocholine; 1-(9Z,12Z-heptadecadienoyl)-2-(11Z-docosenoyl)-glycero-3-phosphocholine; 1-(9Z,12Z-heptadecadienoyl)-2-(13Z,16Z-docosadienoyl)-glycero-3-phosphocholine; 1-(9Z,12Z-heptadecadienoyl)-2-(7Z,10Z,13Z,16Z-docosatetraenoyl)-glycero-3-phosphocholine; 1-octadecanoyl-2-pentadecanoyl-glycero-3-phosphocholine; 1-octadecanoyl-2-heptadecanoyl-glycero-3-phosphocholine; 1-octadecanoyl-2-(9Z-heptadecenoyl)-glycero-3-phosphocholine; 1-octadecanoyl-2-(6Z,9Z,12Z-octadecatrienoyl)-glycero-3-phosphocholine; 1-octadecanoyl-2-(11Z-docosenoyl)-glycero-3-phosphocholine; 1-(9Z-octadecenoyl)-2-dodecanoyl-glycero-3-phosphocholine; 1-(9Z-octadecenoyl)-2-tridecanoyl-glycero-3-phosphocholine; 1-(9Z-octadecenoyl)-2-(9Z-tetradecenoyl)-glycero-3-phosphocholine; 1-(9Z-octadecenoyl)-2-pentadecanoyl-glycero-3-phosphocholine; 1-(9Z-octadecenoyl)-2-(9Z-pentadecenoyl)-glycero-3-phosphocholine; 1-(9Z-octadecenoyl)-2-(9Z-heptadecenoyl)-glycero-3-phosphocholine; 1-(9Z-octadecenoyl)-2-(9Z,12Z-heptadecadienoyl)-glycero-3-phosphocholine; 1-(9Z-octadecenoyl)-2-(6Z,9Z,12Z,15Z-octadecatetraenoyl)-glycero-3-phosphocholine; 1-(9Z-octadecenoyl)-2-nonadecanoyl-glycero-3-phosphocholine; 1-(9Z-octadecenoyl)-2-(11Z-eicosenoyl)-glycero-3-phosphocholine; 1-(9Z-octadecenoyl)-2-(11Z,14Z-eicosadienoyl)-glycero-3-phosphocholine; 1-(9Z-octadecenoyl)-2-(8Z,11Z,14Z-eicosatrienoyl)-glycero-3-phosphocholine; 1-(9Z,12Z-octadecadienoyl)-2-tridecanoyl-glycero-3-phosphocholine; 1-(9Z,12Z-octadecadienoyl)-2-tetradecanoyl-glycero-3-phosphocholine; 1-(9Z,12Z-octadecadienoyl)-2-(9Z-tetradecenoyl)-glycero-3-phosphocholine; 1-(9Z,12Z-octadecadienoyl)-2-pentadecanoyl-glycero-3-phosphocholine; 1-(9Z,12Z-octadecadienoyl)-2-(9Z-hexadecenoyl)-glycero-3-phosphocholine; 1-(9Z,12Z-octadecadienoyl)-2-heptadecanoyl-glycero-3-phosphocholine; 1-(9Z,12Z-octadecadienoyl)-2-(9Z-heptadecenoyl)-glycero-3-phosphocholine; 1-(9Z,12Z-octadecadienoyl)-2-(9Z,12Z-heptadecadienoyl)-glycero-3-phosphocholine; 1-(9Z,12Z-octadecadienoyl)-2-(6Z,9Z,12Z,15Z-octadecatetraenoyl)-glycero-3-phosphocholine; 1-(9Z,12Z-octadecadienoyl)-2-(9Z-nonadecenoyl)-glycero-3-phosphocholine; 1-(9Z,12Z-octadecadienoyl)-2-(11Z-eicosenoyl)-glycero-3-phosphocholine; 1-(9Z,12Z-octadecadienoyl)-2-(11Z,14Z-eicosadienoyl)-glycero-3-phosphocholine; 1-(9Z,12Z-octadecadienoyl)-2-(8Z,11Z,14Z-eicosatrienoyl)-glycero-3-phosphocholine; 1-(9Z,12Z-octadecadienoyl)-2-heneicosanoyl-glycero-3-phosphocholine; 1-(9Z,12Z-octadecadienoyl)-2-docosanoyl-glycero-3-phosphocholine; 1-(9Z,12Z-octadecadienoyl)-2-(11Z-docosenoyl)-glycero-3-phosphocholine; 1-(9Z,12Z-octadecadienoyl)-2-(13Z,16Z-docosadienoyl)-glycero-3-phosphocholine; 1-(6Z,9Z,12Z-octadecatrienoyl)-2-dodecanoyl-glycero-3-phosphocholine; 1-(6Z,9Z,12Z-octadecatrienoyl)-2-tridecanoyl-glycero-3-phosphocholine; 1-(6Z,9Z,12Z-octadecatrienoyl)-2-tetradecanoyl-glycero-3-phosphocholine; 1-(6Z,9Z,12Z-octadecatrienoyl)-2-hexadecanoyl-glycero-3-phosphocholine; 1-(6Z,9Z,12Z-octadecatrienoyl)-2-(9Z-hexadecenoyl)-glycero-3-phosphocholine; 1-(6Z,9Z,12Z-octadecatrienoyl)-2-heptadecanoyl-glycero-3-phosphocholine; 1-(6Z,9Z,12Z-octadecatrienoyl)-2-(9Z-heptadecenoyl)-glycero-3-phosphocholine; 1-(6Z,9Z,12Z-octadecatrienoyl)-2-(9Z,12Z-heptadecadienoyl)-glycero-3-phosphocholine; 1-(6Z,9Z,12Z-octadecatrienoyl)-2-(9Z,12Z-octadecadienoyl)-glycero-3-phosphocholine; 1,2-di-(6Z,9Z,12Z-octadecatrienoyl)-sn-glycero-3-phosphocholine; 1-(6Z,9Z,12Z-octadecatrienoyl)-2-(9Z,12Z,15Z-octadecatrienoyl)-glycero-3-phosphocholine; 1-(6Z,9Z,12Z-octadecatrienoyl)-2-(9Z-nonadecenoyl)-glycero-3-phosphocholine; 1-(6Z,9Z,12Z-octadecatrienoyl)-2-(8Z,11Z,14Z-eicosatrienoyl)-glycero-3-phosphocholine; 1-(6Z,9Z,12Z-octadecatrienoyl)-2-(5Z,8Z,11Z,14Z,17Z-eicosapentaenoyl)-glycero-3-phosphocholine; 1-(6Z,9Z,12Z-octadecatrienoyl)-2-heneicosanoyl-glycero-3-phosphocholine; 1-(6Z,9Z,12Z-octadecatrienoyl)-2-(11Z-docosenoyl)-glycero-3-phosphocholine; 1-(6Z,9Z,12Z-octadecatrienoyl)-2-(13Z,16Z-docosadienoyl)-glycero-3-phosphocholine; 1-(9Z,12Z,15Z-octadecatrienoyl)-2-dodecanoyl-glycero-3-phosphocholine; 1-(9Z,12Z,15Z-octadecatrienoyl)-2-tridecanoyl-glycero-3-phosphocholine; 1-(9Z,12Z,15Z-octadecatrienoyl)-2-(9Z-tetradecenoyl)-glycero-3-phosphocholine; 1-(9Z,12Z,15Z-octadecatrienoyl)-2-(9Z-pentadecenoyl)-glycero-3-phosphocholine; 1-(9Z,12Z,15Z-octadecatrienoyl)-2-hexadecanoyl-glycero-3-phosphocholine; 1-(9Z,12Z,15Z-octadecatrienoyl)-2-(9Z-hexadecenoyl)-glycero-3-phosphocholine; 1-(9Z,12Z,15Z-octadecatrienoyl)-2-heptadecanoyl-glycero-3-phosphocholine; 1-(9Z,12Z,15Z-octadecatrienoyl)-2-(9Z,12Z-heptadecadienoyl)-glycero-3-phosphocholine; 1-(9Z,12Z,15Z-octadecatrienoyl)-2-octadecanoyl-glycero-3-phosphocholine; 1-(9Z,12Z,15Z-octadecatrienoyl)-2-(9Z-nonadecenoyl)-glycero-3-phosphocholine; 1-(9Z,12Z,15Z-octadecatrienoyl)-2-(8Z,11Z,14Z-eicosatrienoyl)-glycero-3-phosphocholine; 1-(9Z,12Z,15Z-octadecatrienoyl)-2-(5Z,8Z,11Z,14Z-eicosatetraenoyl)-glycero-3-phosphocholine; 1-(9Z,12Z,15Z-octadecatrienoyl)-2-(5Z,8Z,11Z,14Z,17Z-eicosapentaenoyl)-glycero-3-phosphocholine; 1-(9Z,12Z,15Z-octadecatrienoyl)-2-(11Z-docosenoyl)-glycero-3-phosphocholine; 1-(6Z,9Z,12Z,15Z-octadecatetraenoyl)-glycero-3-phosphocholine; 1-(6Z,9Z,12Z,15Z-octadecatetraenoyl)-2-dodecanoyl-glycero-3-phosphocholine; 1-(6Z,9Z,12Z,15Z-octadecatetraenoyl)-2-tetradecanoyl-glycero-3-phosphocholine; 1-(6Z,9Z,12Z,15Z-octadecatetraenoyl)-2-pentadecanoyl-glycero-3-phosphocholine; 1-(6Z,9Z,12Z,15Z-octadecatetraenoyl)-2-(9Z-pentadecenoyl)-glycero-3-phosphocholine; 1-(6Z,9Z,12Z,15Z-octadecatetraenoyl)-2-(9Z-hexadecenoyl)-glycero-3-phosphocholine; 1-(6Z,9Z,12Z,15Z-octadecatetraenoyl)-2-(9Z-heptadecenoyl)-glycero-3-phosphocholine; 1-(6Z,9Z,12Z,15Z-octadecatetraenoyl)-2-octadecanoyl-glycero-3-phosphocholine; 1-(6Z,9Z,12Z,15Z-octadecatetraenoyl)-2-(9Z,12Z-octadecadienoyl)-glycero-3-phosphocholine; 1-(6Z,9Z,12Z,15Z-octadecatetraenoyl)-2-(9Z,12Z,15Z-octadecatrienoyl)-glycero-3-phosphocholine; 1,2-di-(6Z,9Z,12Z,15Z-octadecatetraenoyl)-sn-glycero-3-phosphocholine; 1-(6Z,9Z,12Z,15Z-octadecatetraenoyl)-2-eicosanoyl-glycero-3-phosphocholine; 1-(6Z,9Z,12Z,15Z-octadecatetraenoyl)-2-(11Z-eicosenoyl)-glycero-3-phosphocholine; 1-(6Z,9Z,12Z,15Z-octadecatetraenoyl)-2-(11Z,14Z-eicosadienoyl)-glycero-3-phosphocholine; 1-(6Z,9Z,12Z,15Z-octadecatetraenoyl)-2-(8Z,11Z,14Z-eicosatrienoyl)-glycero-3-phosphocholine; 1-(6Z,9Z,12Z,15Z-octadecatetraenoyl)-2-(5Z,8Z,11Z,14Z-eicosatetraenoyl)-glycero-3-phosphocholine; 1-(6Z,9Z,12Z,15Z-octadecatetraenoyl)-2-(5Z,8Z,11Z,14Z,17Z-eicosapentaenoyl)-glycero-3-phosphocholine; 1-(6Z,9Z,12Z,15Z-octadecatetraenoyl)-2-docosanoyl-glycero-3-phosphocholine; 1-(6Z,9Z,12Z,15Z-octadecatetraenoyl)-2-(7Z,10Z,13Z,16Z-docosatetraenoyl)-glycero-3-phosphocholine; 1-nonadecanoyl-2-(9Z-tetradecenoyl)-glycero-3-phosphocholine; 1-nonadecanoyl-2-(9Z-pentadecenoyl)-glycero-3-phosphocholine; 1-nonadecanoyl-2-hexadecanoyl-glycero-3-phosphocholine; 1-nonadecanoyl-2-(9Z-hexadecenoyl)-glycero-3-phosphocholine; 1-nonadecanoyl-2-heptadecanoyl-glycero-3-phosphocholine; 1-nonadecanoyl-2-(9Z-heptadecenoyl)-glycero-3-phosphocholine; 1-nonadecanoyl-2-(9Z,12Z-heptadecadienoyl)-glycero-3-phosphocholine; 1-nonadecanoyl-2-(9Z,12Z,15Z-octadecatrienoyl)-glycero-3-phosphocholine; 1-nonadecanoyl-2-(11Z-eicosenoyl)-glycero-3-phosphocholine; 1-nonadecanoyl-2-(5Z,8Z,11Z,14Z,17Z-eicosapentaenoyl)-glycero-3-phosphocholine; 1-nonadecanoyl-2-heneicosanoyl-glycero-3-phosphocholine; 1-nonadecanoyl-2-(11Z-docosenoyl)-glycero-3-phosphocholine; 1-(9Z-nonadecenoyl)-2-dodecanoyl-glycero-3-phosphocholine; 1-(9Z-nonadecenoyl)-2-tridecanoyl-glycero-3-phosphocholine; 1-(9Z-nonadecenoyl)-2-tetradecanoyl-glycero-3-phosphocholine; 1-(9Z-nonadecenoyl)-2-(9Z-pentadecenoyl)-glycero-3-phosphocholine; 1-(9Z-nonadecenoyl)-2-hexadecanoyl-glycero-3-phosphocholine; 1-(9Z-nonadecenoyl)-2-heptadecanoyl-glycero-3-phosphocholine; 1-(9Z-nonadecenoyl)-2-(9Z-octadecenoyl)-glycero-3-phosphocholine; 1-(9Z-nonadecenoyl)-2-(9Z,12Z,15Z-octadecatrienoyl)-glycero-3-phosphocholine; 1,2-di-(9Z-nonadecenoyl)-sn-glycero-3-phosphocholine; 1-(9Z-nonadecenoyl)-2-eicosanoyl-glycero-3-phosphocholine; 1-(9Z-nonadecenoyl)-2-(8Z,11Z,14Z-eicosatrienoyl)-glycero-3-phosphocholine; 1-(9Z-nonadecenoyl)-2-heneicosanoyl-glycero-3-phosphocholine; 1-(9Z-nonadecenoyl)-2-(11Z-docosenoyl)-glycero-3-phosphocholine; 1-(9Z-nonadecenoyl)-2-(13Z,16Z-docosadienoyl)-glycero-3-phosphocholine; 1-(9Z-nonadecenoyl)-2-(7Z,10Z,13Z,16Z-docosatetraenoyl)-glycero-3-phosphocholine; 1-(9Z-nonadecenoyl)-2-(4Z,7Z,10Z,3Z,1Z,19Z-docosahexaenoyl)-glycero-3-phosphocholine; 1-eicosanoyl-2-(9Z-pentadecenoyl)-glycero-3-phosphocholine; 1-eicosanoyl-2-(9Z-hexadecenoyl)-glycero-3-phosphocholine; 1-eicosanoyl-2-heptadecanoyl-glycero-3-phosphocholine; 1-eicosanoyl-2-(9Z-heptadecenoyl)-glycero-3-phosphocholine; 1-eicosanoyl-2-(9Z,12Z-heptadecadienoyl)-glycero-3-phosphocholine; 1-eicosanoyl-2-(6Z,9Z,12Z,15Z-octadecatetraenoyl)-glycero-3-phosphocholine; 1-eicosanoyl-2-nonadecanoyl-glycero-3-phosphocholine; 1-eicosanoyl-2-(9Z-nonadecenoyl)-glycero-3-phosphocholine; 1-eicosanoyl-2-(8Z,11Z,14Z-eicosatrienoyl)-glycero-3-phosphocholine; 1-eicosanoyl-2-heneicosanoyl-glycero-3-phosphocholine; 1-(11Z-eicosenoyl)-glycero-3-phosphocholine; 1-(11Z-eicosenoyl)-2-tetradecanoyl-glycero-3-phosphocholine; 1-(11Z-eicosenoyl)-2-(9Z-tetradecenoyl)-glycero-3-phosphocholine; 1-(11Z-eicosenoyl)-2-(9Z-hexadecenoyl)-glycero-3-phosphocholine; 1-(11Z-eicosenoyl)-2-(9Z,12Z-octadecadienoyl)-glycero-3-phosphocholine; 1-(11Z-eicosenoyl)-2-(6Z,9Z,12Z-octadecatrienoyl)-glycero-3-phosphocholine; 1-(11Z-eicosenoyl)-2-(9Z,12Z,15Z-octadecatrienoyl)-glycero-3-phosphocholine; 1-(11Z-eicosenoyl)-2-eicosanoyl-glycero-3-phosphocholine; 1-(11Z-eicosenoyl)-2-(8Z,11Z,14Z-eicosatrienoyl)-glycero-3-phosphocholine; 1-(11Z-eicosenoyl)-2-(11Z-docosenoyl)-glycero-3-phosphocholine; 1-(11Z-eicosenoyl)-2-(13Z,16Z-docosadienoyl)-glycero-3-phosphocholine; 1-(11Z,14Z-eicosadienoyl)-glycero-3-phosphocholine; 1-(11Z,14Z-eicosadienoyl)-2-dodecanoyl-glycero-3-phosphocholine; 1-(11Z,14Z-eicosadienoyl)-2-tridecanoyl-glycero-3-phosphocholine; 1-(11Z,14Z-eicosadienoyl)-2-tetradecanoyl-glycero-3-phosphocholine; 1-(11Z,14Z-eicosadienoyl)-2-(9Z-tetradecenoyl)-glycero-3-phosphocholine; 1-(11Z,14Z-eicosadienoyl)-2-pentadecanoyl-glycero-3-phosphocholine; 1-(11Z,14Z-eicosadienoyl)-2-hexadecanoyl-glycero-3-phosphocholine; 1-(11Z,14Z-eicosadienoyl)-2-heptadecanoyl-glycero-3-phosphocholine; 1-(11Z,14Z-eicosadienoyl)-2-(9Z-heptadecenoyl)-glycero-3-phosphocholine; 1-(11Z,14Z-eicosadienoyl)-2-(9Z-octadecenoyl)-glycero-3-phosphocholine; 1-(11Z,14Z-eicosadienoyl)-2-(6Z,9Z,12Z-octadecatrienoyl)-glycero-3-phosphocholine; 1-(11Z,14Z-eicosadienoyl)-2-(9Z,12Z,15Z-octadecatrienoyl)-glycero-3-phosphocholine; 1-(11Z,14Z-eicosadienoyl)-2-(6Z,9Z,12Z,15Z-octadecatetraenoyl)-glycero-3-phosphocholine; 1-(11Z,14Z-eicosadienoyl)-2-nonadecanoyl-glycero-3-phosphocholine; 1,2-di-(11Z,14Z-eicosadienoyl)-sn-glycero-3-phosphocholine; 1-(11Z,14Z-eicosadienoyl)-2-(5Z,8Z,11Z,14Z,17Z-eicosapentaenoyl)-glycero-3-phosphocholine; 1-(11Z,14Z-eicosadienoyl)-2-heneicosanoyl-glycero-3-phosphocholine; 1-(11Z,14Z-eicosadienoyl)-2-docosanoyl-glycero-3-phosphocholine; 1-(11Z,14Z-eicosadienoyl)-2-(7Z,10Z,13Z,16Z-docosatetraenoyl)-glycero-3-phosphocholine; 1-(11Z,14Z-eicosadienoyl)-2-(4Z,7Z,10Z,13Z,16Z,19Z-docosahexaenoyl)-glycero-3-phosphocholine; 1-(8Z,11Z,14Z-eicosatrienoyl)-glycero-3-phosphocholine; 1-(8Z,11Z,14Z-eicosatrienoyl)-2-dodecanoyl-glycero-3-phosphocholine; 1-(8Z,11Z,14Z-eicosatrienoyl)-2-pentadecanoyl-glycero-3-phosphocholine; 1-(8Z,11Z,14Z-eicosatrienoyl)-2-(9Z-pentadecenoyl)-glycero-3-phosphocholine; 1-(8Z,11Z,14Z-eicosatrienoyl)-2-hexadecanoyl-glycero-3-phosphocholine; 1-(8Z,11Z,14Z-eicosatrienoyl)-2-(9Z-hexadecenoyl)-glycero-3-phosphocholine; 1-(8Z,11Z,14Z-eicosatrienoyl)-2-heptadecanoyl-glycero-3-phosphocholine; 1-(8Z,11Z,14Z-eicosatrienoyl)-2-(9Z,12Z-heptadecadienoyl)-glycero-3-phosphocholine; 1-(8Z,11Z,14Z-eicosatrienoyl)-2-octadecanoyl-glycero-3-phosphocholine; 1-(8Z,11Z,14Z-eicosatrienoyl)-2-(9Z-octadecenoyl)-glycero-3-phosphocholine; 1-(8Z,11Z,14Z-eicosatrienoyl)-2-(9Z,12Z-octadecadienoyl)-glycero-3-phosphocholine; 1-(8Z,11Z,14Z-eicosatrienoyl)-2-(9Z,12Z,15Z-octadecatrienoyl)-glycero-3-phosphocholine; 1-(8Z,11Z,14Z-eicosatrienoyl)-2-nonadecanoyl-glycero-3-phosphocholine; 1-(8Z,11Z,14Z-eicosatrienoyl)-2-(9Z-nonadecenoyl)-glycero-3-phosphocholine; 1-(8Z,11Z,14Z-eicosatrienoyl)-2-(11Z,14Z-eicosadienoyl)-glycero-3-phosphocholine; 1,2-di-(8Z,11Z,14Z-eicosatrienoyl)-sn-glycero-3-phosphocholine; 1-(8Z,11Z,14Z-eicosatrienoyl)-2-(5Z,8Z,11Z,14Z-eicosatetraenoyl)-glycero-3-phosphocholine; 1-(8Z,11Z,14Z-eicosatrienoyl)-2-(5Z,8Z,11Z,14Z,17Z-eicosapentaenoyl)-glycero-3-phosphocholine; 1-(8Z,11Z,14Z-eicosatrienoyl)-2-docosanoyl-glycero-3-phosphocholine; 1-(8Z,11Z,14Z-eicosatrienoyl)-2-(13Z,16Z-docosadienoyl)-glycero-3-phosphocholine; 1-(8Z,11Z,14Z-eicosatrienoyl)-2-(7Z,10Z,13Z,16Z-docosatetraenoyl)-glycero-3-phosphocholine; 1-(5Z,8Z,11Z,14Z-eicosatetraenoyl)-2-tetradecanoyl-glycero-3-phosphocholine; 1-(5Z,8Z,11Z,14Z-eicosatetraenoyl)-2-pentadecanoyl-glycero-3-phosphocholine; 1-(5Z,8Z,11Z,14Z-eicosatetraenoyl)-2-heptadecanoyl-glycero-3-phosphocholine; 1-(5Z,8Z,11Z,14Z-eicosatetraenoyl)-2-(9Z-heptadecenoyl)-glycero-3-phosphocholine; 1-(5Z,8Z,11Z,14Z-eicosatetraenoyl)-2-(9Z,12Z-heptadecadienoyl)-glycero-3-phosphocholine; 1-(5Z,8Z,11Z,14Z-eicosatetraenoyl)-2-(9Z-octadecenoyl)-glycero-3-phosphocholine; 1-(5Z,8Z,11Z,14Z-eicosatetraenoyl)-2-(9Z,12Z-octadecadienoyl)-glycero-3-phosphocholine; 1-(5Z,8Z,11Z,14Z-eicosatetraenoyl)-2-eicosanoyl-glycero-3-phosphocholine; 1-(5Z,8Z,11Z,14Z-eicosatetraenoyl)-2-(11Z,14Z-eicosadienoyl)-glycero-3-phosphocholine; 1-(5Z,8Z,11Z,14Z-eicosatetraenoyl)-2-(8Z,11Z,14Z-eicosatrienoyl)-glycero-3-phosphocholine; 1-(5Z,8Z,11Z,14Z-eicosatetraenoyl)-2-docosanoyl-glycero-3-phosphocholine; 1-(5Z,8Z,11Z,14Z-eicosatetraenoyl)-2-(13Z,16Z-docosadienoyl)-glycero-3-phosphocholine; 1-(5Z,8Z,11Z,14Z-eicosatetraenoyl)-2-(7Z,10Z,13Z,16Z-docosatetraenoyl)-glycero-3-phosphocholine; 1-(5Z,8Z,11Z,14Z,17Z-eicosapentaenoyl)-2-tetradecanoyl-glycero-3-phosphocholine; 1-(5Z,8Z,11Z,14Z,17Z-eicosapentaenoyl)-2-(9Z-tetradecenoyl)-glycero-3-phosphocholine; 1-(5Z,8Z,11Z,14Z,17Z-eicosapentaenoyl)-2-hexadecanoyl-glycero-3-phosphocholine; 1-(5Z,8Z,11Z,14Z,17Z-eicosapentaenoyl)-2-heptadecanoyl-glycero-3-phosphocholine; 1-(5Z,8Z,11Z,14Z,17Z-eicosapentaenoyl)-2-octadecanoyl-glycero-3-phosphocholine; 1-(5Z,8Z,11Z,14Z,17Z-eicosapentaenoyl)-2-(9Z,12Z-octadecadienoyl)-glycero-3-phosphocholine; 1-(5Z,8Z,11Z,14Z,17Z-eicosapentaenoyl)-2-(6Z,9Z,12Z-octadecatrienoyl)-glycero-3-phosphocholine; 1-(5Z,8Z,11Z,14Z,17Z-eicosapentaenoyl)-2-(9Z,12Z,15Z-octadecatrienoyl)-glycero-3-phosphocholine; 1-(5Z,8Z,11Z,14Z,17Z-eicosapentaenoyl)-2-(6Z,9Z,12Z,15Z-octadecatetraenoyl)-glycero-3-phosphocholine; 1-(5Z,8Z,11Z,14Z,17Z-eicosapentaenoyl)-2-nonadecanoyl-glycero-3-phosphocholine; 1-(5Z,8Z,11Z,14Z,17Z-eicosapentaenoyl)-2-(11Z-eicosenoyl)-glycero-3-phosphocholine; 1-(5Z,8Z,11Z,14Z,17Z-eicosapentaenoyl)-2-(11Z,14Z-eicosadienoyl)-glycero-3-phosphocholine; 1-(5Z,8Z,11Z,14Z,17Z-eicosapentaenoyl)-2-(5Z,8Z,11Z,14Z-eicosatetraenoyl)-glycero-3-phosphocholine; 1,2-di-(5Z,8Z,11Z,14Z,17Z-eicosapentaenoyl)-sn-glycero-3-phosphocholine; 1-(5Z,8Z,11Z,14Z,17Z-eicosapentaenoyl)-2-heneicosanoyl-glycero-3-phosphocholine; 1-(5Z,8Z,11Z,14Z,17Z-eicosapentaenoyl)-2-(13Z,16Z-docosadienoyl)-glycero-3-phosphocholine; 1-(5Z,8Z,11Z,14Z,17Z-eicosapentaenoyl)-2-(7Z,10Z,13Z,16Z-docosatetraenoyl)-glycero-3-phosphocholine; 1-heneicosanoyl-2-(9Z-tetradecenoyl)-glycero-3-phosphocholine; 1-heneicosanoyl-2-pentadecanoyl-glycero-3-phosphocholine; 1-heneicosanoyl-2-(9Z-pentadecenoyl)-glycero-3-phosphocholine; 1-heneicosanoyl-2-(9Z-heptadecenoyl)-glycero-3-phosphocholine; 1-heneicosanoyl-2-(9Z,12Z-octadecadienoyl)-glycero-3-phosphocholine; 1-heneicosanoyl-2-(6Z,9Z,12Z,15Z-octadecatetraenoyl)-glycero-3-phosphocholine; 1-heneicosanoyl-2-nonadecanoyl-glycero-3-phosphocholine; 1-heneicosanoyl-2-eicosanoyl-glycero-3-phosphocholine; 1-heneicosanoyl-2-(11Z,14Z-eicosadienoyl)-glycero-3-phosphocholine; 1-heneicosanoyl-2-(5Z,8Z, 11Z,14Z-eicosatetraenoyl)-glycero-3-phosphocholine; 1-heneicosanoyl-2-(5Z,8Z,11Z,14Z,17Z-eicosapentaenoyl)-glycero-3-phosphocholine; 1-heneicosanoyl-2-docosanoyl-glycero-3-phosphocholine; 1-heneicosanoyl-2-(13Z,16Z-docosadienoyl)-glycero-3-phosphocholine; 1-heneicosanoyl-2-(7Z,10Z,13Z,16Z-docosatetraenoyl)-glycero-3-phosphocholine; 1-docosanoyl-2-(9Z-hexadecenoyl)-glycero-3-phosphocholine; 1-docosanoyl-2-heptadecanoyl-glycero-3-phosphocholine; 1-docosanoyl-2-(9Z-heptadecenoyl)-glycero-3-phosphocholine; 1-docosanoyl-2-(6Z,9Z,12Z-octadecatrienoyl)-glycero-3-phosphocholine; 1-docosanoyl-2-(9Z,12Z,15Z-octadecatrienoyl)-glycero-3-phosphocholine; 1-docosanoyl-2-(6Z,9Z,12Z,15Z-octadecatetraenoyl)-glycero-3-phosphocholine; 1-docosanoyl-2-(11Z,14Z-eicosadienoyl)-glycero-3-phosphocholine; 1-docosanoyl-2-(5Z,8Z,11Z,14Z,17Z-eicosapentaenoyl)-glycero-3-phosphocholine; 1-docosanoyl-2-heneicosanoyl-glycero-3-phosphocholine; 1-docosanoyl-2-(11Z-docosenoyl)-glycero-3-phosphocholine; 1-docosanoyl-2-(13Z,16Z-docosadienoyl)-glycero-3-phosphocholine; 1-docosanoyl-2-(7Z,10Z,13Z,16Z-docosatetraenoyl)-glycero-3-phosphocholine; 1-(11Z-docosenoyl)-2-dodecanoyl-glycero-3-phosphocholine; 1-(11Z-docosenoyl)-2-tridecanoyl-glycero-3-phosphocholine; 1-(11Z-docosenoyl)-2-(9Z-tetradecenoyl)-glycero-3-phosphocholine; 1-(11Z-docosenoyl)-2-pentadecanoyl-glycero-3-phosphocholine; 1-(11Z-docosenoyl)-2-heptadecanoyl-glycero-3-phosphocholine; 1-(11Z-docosenoyl)-2-(9Z,12Z-heptadecadienoyl)-glycero-3-phosphocholine; 1-(11Z-docosenoyl)-2-octadecanoyl-glycero-3-phosphocholine; 1-(11Z-docosenoyl)-2-(9Z-octadecenoyl)-glycero-3-phosphocholine; 1-(11Z-docosenoyl)-2-(9Z,12Z-octadecadienoyl)-glycero-3-phosphocholine; 1-(11Z-docosenoyl)-2-(6Z,9Z,12Z-octadecatrienoyl)-glycero-3-phosphocholine; 1-(11Z-docosenoyl)-2-(9Z,12Z,15Z-octadecatrienoyl)-glycero-3-phosphocholine; 1-(11Z-docosenoyl)-2-(9Z-nonadecenoyl)-glycero-3-phosphocholine; 1-(11Z-docosenoyl)-2-(11Z-eicosenoyl)-glycero-3-phosphocholine; 1-(11Z-docosenoyl)-2-(5Z,8Z,11Z,14Z-eicosatetraenoyl)-glycero-3-phosphocholine; 1-(11Z-docosenoyl)-2-heneicosanoyl-glycero-3-phosphocholine; 1,2-di-(11Z-docosenoyl)-sn-glycero-3-phosphocholine; 1-(11Z-docosenoyl)-2-(13Z,16Z-docosadienoyl)-glycero-3-phosphocholine; 1-(11Z-docosenoyl)-2-(7Z,10Z,13Z,16Z-docosatetraenoyl)-glycero-3-phosphocholine; 1-(13Z,16Z-docosadienoyl)-glycero-3-phosphocholine; 1-(13Z,16Z-docosadienoyl)-2-dodecanoyl-glycero-3-phosphocholine; 1-(13Z,16Z-docosadienoyl)-2-tridecanoyl-glycero-3-phosphocholine; 1-(13Z,16Z-docosadienoyl)-2-tetradecanoyl-glycero-3-phosphocholine; 1-(13Z,16Z-docosadienoyl)-2-(9Z-heptadecenoyl)-glycero-3-phosphocholine; 1-(13Z,16Z-docosadienoyl)-2-octadecanoyl-glycero-3-phosphocholine; 1-(13Z,16Z-docosadienoyl)-2-(9Z,12Z,15Z-octadecatrienoyl)-glycero-3-phosphocholine; 1-(13Z,16Z-docosadienoyl)-2-(6Z,9Z,12Z,15Z-octadecatetraenoyl)-glycero-3-phosphocholine; 1-(13Z,16Z-docosadienoyl)-2-nonadecanoyl-glycero-3-phosphocholine; 1-(13Z,16Z-docosadienoyl)-2-(9Z-nonadecenoyl)-glycero-3-phosphocholine; 1-(13Z,16Z-docosadienoyl)-2-(5Z,8Z,11Z,14Z-eicosatetraenoyl)-glycero-3-phosphocholine; 1-(13Z,16Z-docosadienoyl)-2-(5Z,8Z,11Z,14Z,17Z-eicosapentaenoyl)-glycero-3-phosphocholine; 1-(13Z,16Z-docosadienoyl)-2-(11Z-docosenoyl)-glycero-3-phosphocholine; 1-(13Z,16Z-docosadienoyl)-2-(7Z,10Z,13Z,16Z-docosatetraenoyl)-glycero-3-phosphocholine; 1-(7Z,10Z,13Z,16Z-docosatetraenoyl)-2-tetradecanoyl-glycero-3-phosphocholine; 1-(7Z,10Z,13Z,16Z-docosatetraenoyl)-2-(9Z-tetradecenoyl)-glycero-3-phosphocholine; 1-(7Z,10Z,13Z,16Z-docosatetraenoyl)-2-(9Z-pentadecenoyl)-glycero-3-phosphocholine; 1-(7Z,10Z,13Z,16Z-docosatetraenoyl)-2-hexadecanoyl-glycero-3-phosphocholine; 1-(7Z,10Z,13Z,16Z-docosatetraenoyl)-2-(9Z,12Z-heptadecadienoyl)-glycero-3-phosphocholine; 1-(7Z,10Z,13Z,16Z-docosatetraenoyl)-2-octadecanoyl-glycero-3-phosphocholine; 1-(7Z,10Z,13Z,16Z-docosatetraenoyl)-2-(9Z-octadecenoyl)-glycero-3-phosphocholine; 1-(7Z,10Z,13Z,16Z-docosatetraenoyl)-2-(9Z,12Z,15Z-octadecatrienoyl)-glycero-3-phosphocholine; 1-(7Z,10Z,13Z,16Z-docosatetraenoyl)-2-nonadecanoyl-glycero-3-phosphocholine; 1-(7Z,10Z,13Z,16Z-docosatetraenoyl)-2-(9Z-nonadecenoyl)-glycero-3-phosphocholine; 1-(7Z,10Z,13Z,16Z-docosatetraenoyl)-2-(8Z,11Z,14Z-eicosatrienoyl)-glycero-3-phosphocholine; 1-(7Z,10Z,13Z,16Z-docosatetraenoyl)-2-(5Z,8Z,11Z,14Z,17Z-eicosapentaenoyl)-glycero-3-phosphocholine; 1-(7Z,10Z,13Z,16Z-docosatetraenoyl)-2-heneicosanoyl-glycero-3-phosphocholine; 1-(7Z,10Z,13Z,16Z-docosatetraenoyl)-2-docosanoyl-glycero-3-phosphocholine; 1-(7Z,10Z,13Z,16Z-docosatetraenoyl)-2-(11Z-docosenoyl)-glycero-3-phosphocholine; 1,2-di-(7Z,10Z,13Z,16Z-docosatetraenoyl)-sn-glycero-3-phosphocholine; 1-(7Z,10Z,13Z,16Z-docosatetraenoyl)-2-(4Z,7Z,10Z,13Z,16Z,19Z-docosahexaenoyl)-glycero-3-phosphocholine; 1-(4Z,7Z,10Z,13Z,16Z,19Z-docosahexaenoyl)-2-tetradecanoyl-glycero-3-phosphocholine; 1-(4Z,7Z,10Z,13Z,16Z,19Z-docosahexaenoyl)-2-(9Z-tetradecenoyl)-glycero-3-phosphocholine; 1-(4Z,7Z,10Z,13Z,16Z,19Z-docosahexaenoyl)-2-pentadecanoyl-glycero-3-phosphocholine; 1-(4Z,7Z,10Z,13Z,16Z,19Z-docosahexaenoyl)-2-(9Z-pentadecenoyl)-glycero-3-phosphocholine; 1-(4Z,7Z,10Z,13Z,16Z,19Z-docosahexaenoyl)-2-(9Z-heptadecenoyl)-glycero-3-phosphocholine; 1-(4Z,7Z,10Z,13Z,16Z,19Z-docosahexaenoyl)-2-(9Z,12Z-heptadecadienoyl)-glycero-3-phosphocholine; 1-(4Z,7Z,10Z,13Z,16Z,19Z-docosahexaenoyl)-2-octadecanoyl-glycero-3-phosphocholine; 1-(4Z,7Z,10Z,13Z,16Z,19Z-docosahexaenoyl)-2-eicosanoyl-glycero-3-phosphocholine; 1-(4Z,7Z,10Z,13Z,16Z,19Z-docosahexaenoyl)-2-(11Z-eicosenoyl)-glycero-3-phosphocholine; 1-(4Z,7Z,10Z,13Z,16Z,19Z-docosahexaenoyl)-2-(11Z,14Z-eicosadienoyl)-glycero-3-phosphocholine; 1-(4Z,7Z,10Z,13Z,16Z,19Z-docosahexaenoyl)-2-(5Z,8Z,11Z,14Z-eicosatetraenoyl)-glycero-3-phosphocholine; 1-(4Z,7Z,10Z,13Z,16Z,19Z-docosahexaenoyl)-2-(5Z,8Z,11Z,14Z,17Z-eicosapentaenoyl)-glycero-3-phosphocholine; 1-(4Z,7Z,10Z,13Z,16Z,19Z-docosahexaenoyl)-2-docosanoyl-glycero-3-phosphocholine; 1-(4Z,7Z,10Z,13Z,16Z,19Z-docosahexaenoyl)-2-(13Z,16Z-docosadienoyl)-glycero-3-phosphocholine; 1-(4Z,7Z,10Z,13Z,16Z,19Z-docosahexaenoyl)-2-(7Z,10Z,13Z,16Z-docosatetraenoyl)-glycero-3-phosphocholine; 1-hexadecyl-2-tridecanoyl-glycero-3-phosphocholine; 1-hexadecyl-2-(9Z-heptadecenoyl)-glycero-3-phosphocholine; 1-hexadecyl-2-(11Z,14Z-eicosadienoyl)-glycero-3-phosphocholine; 1-hexadecyl-2-heneicosanoyl-glycero-3-phosphocholine; 1-hexadecyl-2-(7Z,10Z,13Z,16Z-docosatetraenoyl)-glycero-3-phosphocholine; 1-octadecyl-2-dodecanoyl-glycero-3-phosphocholine; 1-octadecyl-2-(9Z-pentadecenoyl)-glycero-3-phosphocholine; 1-octadecyl-2-(9Z-heptadecenoyl)-glycero-3-phosphocholine; 1-octadecyl-2-(9Z,12Z-heptadecadienoyl)-glycero-3-phosphocholine; 1-octadecyl-2-(9Z-octadecenoyl)-glycero-3-phosphocholine; 1-octadecyl-2-(9Z,12Z-octadecadienoyl)-glycero-3-phosphocholine; 1-octadecyl-2-(6Z,9Z,12Z,15Z-octadecatetraenoyl)-glycero-3-phosphocholine; 1-octadecyl-2-nonadecanoyl-glycero-3-phosphocholine; 1-octadecyl-2-heneicosanoyl-glycero-3-phosphocholine; 1-octadecyl-2-(13Z,16Z-docosadienoyl)-glycero-3-phosphocholine; 1-octadecyl-2-(7Z,10Z,13Z,16Z-docosatetraenoyl)-glycero-3-phosphocholine; 1-eicosyl-2-dodecanoyl-glycero-3-phosphocholine; 1-eicosyl-2-tetradecanoyl-glycero-3-phosphocholine; 1-eicosyl-2-(9Z-tetradecenoyl)-glycero-3-phosphocholine; 1-eicosyl-2-pentadecanoyl-glycero-3-phosphocholine; 1-eicosyl-2-(9Z-pentadecenoyl)-glycero-3-phosphocholine; 1-eicosyl-2-octadecanoyl-glycero-3-phosphocholine; 1-eicosyl-2-(9Z,12Z-octadecadienoyl)-glycero-3-phosphocholine; 1-eicosyl-2-(6Z,9Z,12Z-octadecatrienoyl)-glycero-3-phosphocholine; 1-eicosyl-2-(9Z,12Z,15Z-octadecatrienoyl)-glycero-3-phosphocholine; 1-eicosyl-2-nonadecanoyl-glycero-3-phosphocholine; 1-eicosyl-2-eicosanoyl-glycero-3-phosphocholine; 1-eicosyl-2-(11Z-eicosenoyl)-glycero-3-phosphocholine; 1-eicosyl-2-(8Z,11Z,14Z-eicosatrienoyl)-glycero-3-phosphocholine; 1-eicosyl-2-(5Z,8Z,11Z,14Z-eicosatetraenoyl)-glycero-3-phosphocholine; 1-(1Z-hexadecenyl)-2-dodecanoyl-glycero-3-phosphocholine; 1-(1Z-hexadecenyl)-2-tridecanoyl-glycero-3-phosphocholine; 1-(1Z-hexadecenyl)-2-tetradecanoyl-glycero-3-phosphocholine; 1-(1Z-hexadecenyl)-2-pentadecanoyl-glycero-3-phosphocholine; 1-(1Z-hexadecenyl)-2-hexadecanoyl-glycero-3-phosphocholine; 1-(1Z-hexadecenyl)-2-heptadecanoyl-glycero-3-phosphocholine; 1-(1Z-hexadecenyl)-2-(9Z,12Z-heptadecadienoyl)-glycero-3-phosphocholine; 1-(1Z-hexadecenyl)-2-(6Z,9Z,12Z-octadecatrienoyl)-glycero-3-phosphocholine; 1-(1Z-hexadecenyl)-2-(9Z,12Z,15Z-octadecatrienoyl)-glycero-3-phosphocholine; 1-(1Z-hexadecenyl)-2-(11Z-eicosenoyl)-glycero-3-phosphocholine; 1-(1Z-hexadecenyl)-2-(11Z,14Z-eicosadienoyl)-glycero-3-phosphocholine; 1-(1Z-hexadecenyl)-2-(5Z,8Z,11Z,14Z,17Z-eicosapentaenoyl)-glycero-3-phosphocholine; 1-(1Z-hexadecenyl)-2-(7Z,10Z,13Z,16Z-docosatetraenoyl)-glycero-3-phosphocholine; 1-(1Z-octadecenyl)-2-(9Z-pentadecenoyl)-glycero-3-phosphocholine; 1-(1Z-octadecenyl)-2-hexadecanoyl-glycero-3-phosphocholine; 1-(1Z-octadecenyl)-2-(9Z-hexadecenoyl)-glycero-3-phosphocholine; 1-(1Z-octadecenyl)-2-octadecanoyl-glycero-3-phosphocholine; 1-(1Z-octadecenyl)-2-(6Z,9Z,12Z-octadecatrienoyl)-glycero-3-phosphocholine; 1-(1Z-octadecenyl)-2-(9Z,12Z,15Z-octadecatrienoyl)-glycero-3-phosphocholine; 1-(1Z-octadecenyl)-2-(6Z,9Z,12Z,15Z-octadecatetraenoyl)-glycero-3-phosphocholine; 1-(1Z-octadecenyl)-2-nonadecanoyl-glycero-3-phosphocholine; 1-(1Z-octadecenyl)-2-(9Z-nonadecenoyl)-glycero-3-phosphocholine; 1-(1Z-octadecenyl)-2-(11Z,14Z-eicosadienoyl)-glycero-3-phosphocholine; 1-(1Z-octadecenyl)-2-heneicosanoyl-glycero-3-phosphocholine; 1-(1Z-octadecenyl)-2-(13Z,16Z-docosadienoyl)-glycero-3-phosphocholine; 1-(1Z-eicosenyl)-2-tetradecanoyl-glycero-3-phosphocholine; 1-(1Z-eicosenyl)-2-(9Z-pentadecenoyl)-glycero-3-phosphocholine; 1-(1Z-eicosenyl)-2-hexadecanoyl-glycero-3-phosphocholine; 1-(1Z-eicosenyl)-2-heptadecanoyl-glycero-3-phosphocholine; 1-(1Z-eicosenyl)-2-(9Z-heptadecenoyl)-glycero-3-phosphocholine; 1-(1Z-eicosenyl)-2-(9Z,12Z-heptadecadienoyl)-glycero-3-phosphocholine; 1-(1Z-eicosenyl)-2-octadecanoyl-glycero-3-phosphocholine; 1-(1Z-eicosenyl)-2-(9Z,12Z-octadecadienoyl)-glycero-3-phosphocholine; 1-(1Z-eicosenyl)-2-(6Z,9Z,12Z,15Z-octadecatetraenoyl)-glycero-3-phosphocholine; 1-(1Z-eicosenyl)-2-(9Z-nonadecenoyl)-glycero-3-phosphocholine; 1-(1Z-eicosenyl)-2-(11Z,14Z-eicosadienoyl)-glycero-3-phosphocholine; 1-(1Z-eicosenyl)-2-(5Z,8Z,11Z,14Z,17Z-eicosapentaenoyl)-glycero-3-phosphocholine; 1-(1Z-eicosenyl)-2-(13Z,16Z-docosadienoyl)-glycero-3-phosphocholine; 1-(1Z-eicosenyl)-2-(7Z,10Z,13Z,16Z-docosatetraenoyl)-glycero-3-phosphocholine; 1-(1Z-eicosenyl)-2-(4Z,7Z,10Z,13Z,16Z,19Z-docosahexaenoyl)-glycero-3-phosphocholine; 1-tetradecanoyl-2-(8Z,11Z,14Z,17Z-eicosatetraenoyl)-sn-glycero-3-phosphocholine; 1-tetradecanoyl-2-(4Z,7Z,10Z,13Z,16Z-docosapentaenoyl)-sn-glycero-3-phosphocholine; 1-(9Z-tetradecenoyl)-2-(7Z,10Z,13Z,16Z,19Z-docosapentaenoyl)-sn-glycero-3-phosphocholine; 1-hexadecanoyl-2-(8Z,11Z,14Z,17Z-eicosatetraenoyl)-sn-glycero-3-phosphocholine; 1-hexadecanoyl-2-(4Z,7Z,10Z,13Z,16Z,19Z-docosahexaenoyl)-sn-glycero-3-phosphocholine; 1-(9Z-hexadecenoyl)-2-(8Z,11Z,14Z,17Z-eicosatetraenoyl)-sn-glycero-3-phosphocholine; 1-(9Z-hexadecenoyl)-2-(7Z,10Z,13Z,16Z,19Z-docosapentaenoyl)-sn-glycero-3-phosphocholine; 1-(9Z-hexadecenoyl)-2-(15Z-tetracosenoyl)-sn-glycero-3-phosphocholine; 1-octadecanoyl-2-(8Z,11Z,14Z,17Z-eicosatetraenoyl)-sn-glycero-3-phosphocholine; 1-(11Z-octadecenoyl)-2-hexadecanoyl-sn-glycero-3-phosphocholine; 1-(11Z-octadecenoyl)-2-(9Z,12Z-octadecadienoyl)-sn-glycero-3-phosphocholine; 1-(11Z-octadecenoyl)-2-(9Z,12Z,15Z-octadecatrienoyl)-sn-glycero-3-phosphocholine; 1-(11Z-octadecenoyl)-2-(5Z,8Z,11Z-eicosatrienoyl)-sn-glycero-3-phosphocholine; 1-(11Z-octadecenoyl)-2-(5Z,8Z,11Z,14Z-eicosatetraenoyl)-sn-glycero-3-phosphocholine; 1-(11Z-octadecenoyl)-2-(13Z-docosenoyl)-sn-glycero-3-phosphocholine; 1-(11Z-octadecenoyl)-2-(7Z,10Z,13Z,16Z-docosatetraenoyl)-sn-glycero-3-phosphocholine; 1-(11Z-octadecenoyl)-2-tetracosanoyl-sn-glycero-3-phosphocholine; 1-(11Z-octadecenoyl)-2-(15Z-tetracosenoyl)-sn-glycero-3-phosphocholine; 1-(9Z-octadecenoyl)-2-(13Z-docosenoyl)-sn-glycero-3-phosphocholine; 1-(9Z-octadecenoyl)-2-(15Z-tetracosenoyl)-sn-glycero-3-phosphocholine; 1-(9Z,12Z-octadecadienoyl)-2-tetracosanoyl-sn-glycero-3-phosphocholine; 1-(6Z,9Z,12Z-octadecatrienoyl)-2-(15Z-tetracosenoyl)-sn-glycero-3-phosphocholine; 1-(9Z,12Z,15Z-octadecatrienoyl)-2-(11Z-octadecenoyl)-sn-glycero-3-phosphocholine; 1-(9Z,12Z,15Z-octadecatrienoyl)-2-(5Z,8Z,11Z-eicosatrienoyl)-sn-glycero-3-phosphocholine; 1-(9Z,12Z,15Z-octadecatrienoyl)-2-(13Z-docosenoyl)-sn-glycero-3-phosphocholine; 1-(9Z,12Z,15Z-octadecatrienoyl)-2-(15Z-tetracosenoyl)-sn-glycero-3-phosphocholine; 1-(6Z,9Z,12Z,15Z-octadecatetraenoyl)-2-(11Z-octadecenoyl)-sn-glycero-3-phosphocholine; 1-(6Z,9Z,12Z,15Z-octadecatetraenoyl)-2-tetracosanoyl-sn-glycero-3-phosphocholine; 1-(11Z-eicosenoyl)-2-(8Z,11Z,14Z,17Z-eicosatetraenoyl)-sn-glycero-3-phosphocholine; 1-(11Z-eicosenoyl)-2-(13Z-docosenoyl)-sn-glycero-3-phosphocholine; 1-(11Z,14Z-eicosadienoyl)-2-(11Z-octadecenoyl)-sn-glycero-3-phosphocholine; 1-(5Z,8Z,11Z-eicosatrienoyl)-2-tetradecanoyl-sn-glycero-3-phosphocholine; 1-(5Z,8Z,11Z-eicosatrienoyl)-2-(9Z-tetradecenoyl)-sn-glycero-3-phosphocholine; 1-(5Z,8Z,11Z-eicosatrienoyl)-2-octadecanoyl-sn-glycero-3-phosphocholine; 1-(5Z,8Z,11Z-eicosatrienoyl)-2-(9Z,12Z-octadecadienoyl)-sn-glycero-3-phosphocholine; 1-(5Z,8Z,11Z-eicosatrienoyl)-2-(6Z,9Z,12Z-octadecatrienoyl)-sn-glycero-3-phosphocholine; 1-(5Z,8Z,11Z-eicosatrienoyl)-2-(11Z,14Z-eicosadienoyl)-sn-glycero-3-phosphocholine; 1-(5Z,8Z,11Z-eicosatrienoyl)-2-(13Z-docosenoyl)-sn-glycero-3-phosphocholine; 1-(8Z,11Z,14Z-eicosatrienoyl)-2-(11Z-octadecenoyl)-sn-glycero-3-phosphocholine; 1-(8Z,11Z,14Z-eicosatrienoyl)-2-(13Z-docosenoyl)-sn-glycero-3-phosphocholine; 1-(8Z,11Z,14Z,17Z-eicosatetraenoyl)-2-(11Z-octadecenoyl)-sn-glycero-3-phosphocholine; 1-(8Z,11Z,14Z,17Z-eicosatetraenoyl)-2-(9Z,12Z-octadecadienoyl)-sn-glycero-3-phosphocholine; 1-(8Z,11Z,14Z,17Z-eicosatetraenoyl)-2-eicosanoyl-sn-glycero-3-phosphocholine; 1-(8Z,11Z,14Z,17Z-eicosatetraenoyl)-2-docosanoyl-sn-glycero-3-phosphocholine; 1-(5Z,8Z,11Z,14Z,17Z-eicosapentaenoyl)-2-(11Z-octadecenoyl)-sn-glycero-3-phosphocholine; 1-docosanoyl-2-(8Z,11Z,14Z,17Z-eicosatetraenoyl)-sn-glycero-3-phosphocholine; 1-(13Z-docosenoyl)-2-tetradecanoyl-sn-glycero-3-phosphocholine; 1-(13Z-docosenoyl)-2-(11Z-octadecenoyl)-sn-glycero-3-phosphocholine; 1-(13Z-docosenoyl)-2-(9Z,12Z-octadecadienoyl)-sn-glycero-3-phosphocholine; 1-(13Z-docosenoyl)-2-eicosanoyl-sn-glycero-3-phosphocholine; 1-(13Z-docosenoyl)-2-(11Z-eicosenoyl)-sn-glycero-3-phosphocholine; 1-(7Z,10Z,13Z,16Z-docosatetraenoyl)-2-(11Z-octadecenoyl)-sn-glycero-3-phosphocholine; 1-(4Z,7Z,10Z,13Z,16Z-docosapentaenoyl)-2-tetradecanoyl-sn-glycero-3-phosphocholine; 1-(4Z,7Z,10Z,13Z,16Z-docosapentaenoyl)-2-(9Z-tetradecenoyl)-sn-glycero-3-phosphocholine; 1-(7Z,10Z,13Z,16Z,19Z-docosapentaenoyl)-2-(9Z-tetradecenoyl)-sn-glycero-3-phosphocholine; 1-tetracosanoyl-2-(11Z-octadecenoyl)-sn-glycero-3-phosphocholine; 1-tetracosanoyl-2-(6Z,9Z,12Z,15Z-octadecatetraenoyl)-sn-glycero-3-phosphocholine; 1-(15Z-tetracosenoyl)-2-(9Z-octadecenoyl)-sn-glycero-3-phosphocholine; 1-(15Z-tetracosenoyl)-2-(6Z,9Z,12Z-octadecatrienoyl)-sn-glycero-3-phosphocholine; 1-(9Z-hexadecenyl)-2-octadecanoyl-sn-glycero-3-phosphocholine; 1-(9Z-hexadecenyl)-2-(9Z,12Z-octadecadienoyl)-sn-glycero-3-phosphocholine; 1-(9Z-hexadecenyl)-2-eicosanoyl-sn-glycero-3-phosphocholine; 1-(9Z-hexadecenyl)-2-(8Z,11Z,14Z,17Z-eicosatetraenoyl)-sn-glycero-3-phosphocholine; 1-octadecyl-2-(8Z,11Z,14Z,17Z-eicosatetraenoyl)-sn-glycero-3-phosphocholine; 1-(11Z-octadecenyl)-2-hexadecanoyl-sn-glycero-3-phosphocholine; 1-(9Z,12Z-octadecadienyl)-2-(9Z,12Z-octadecadienoyl)-sn-glycero-3-phosphocholine; 1-(9Z,12Z-octadecadienyl)-2-eicosanoyl-sn-glycero-3-phosphocholine; 1-(9Z,12Z-octadecadienyl)-2-docosanoyl-sn-glycero-3-phosphocholine; 1-(9Z,12Z-octadecadienyl)-2-tetracosanoyl-sn-glycero-3-phosphocholine; 1-eicosyl-2-(8Z,11Z,14Z,17Z-eicosatetraenoyl)-sn-glycero-3-phosphocholine; 1-docosyl-2-(11Z-eicosenoyl)-sn-glycero-3-phosphocholine; 1-docosyl-2-(10Z,13Z,16Z-docosatrienoyl)-sn-glycero-3-phosphocholine; 1-(13Z,16Z-docosenyl)-2-(13Z,16Z-docosadienoyl)-sn-glycero-3-phosphocholine; 1-(13Z,16Z-docosenyl)-2-(10Z,13Z,16Z-docosatrienoyl)-sn-glycero-3-phosphocholine; 1-tetracosyl-2-(9Z,12Z,15Z-octadecatrienoyl)-sn-glycero-3-phosphocholine; 1-tetracosyl-2-(8Z,11Z,14Z,17Z-eicosatetraenoyl)-sn-glycero-3-phosphocholine; 1-(1Z-hexadecenyl)-2-(11Z-octadecenoyl)-sn-glycero-3-phosphocholine; 1-(1Z-octadecenyl)-2-(15Z-tetracosenoyl)-sn-glycero-3-phosphocholine; 1-(1Z,11Z-octadecadienyl)-2-octadecanoyl-sn-glycero-3-phosphocholine; 1-(1Z,11Z-octadecadienyl)-2-(11Z-octadecenoyl)-sn-glycero-3-phosphocholine; 1-(1Z,11Z-octadecadienyl)-2-(11Z,14Z-eicosadienoyl)-sn-glycero-3-phosphocholine; 1-(1Z,11Z-octadecadienyl)-2-(13Z-docosenoyl)-sn-glycero-3-phosphocholine; 1-(1Z,11Z-octadecadienyl)-2-(13Z,16Z-docosadienoyl)-sn-glycero-3-phosphocholine; 1-(1Z,11Z-octadecadienyl)-2-tetracosanoyl-sn-glycero-3-phosphocholine; 1-(1Z,11Z-octadecadienyl)-2-(15Z-tetracosenoyl)-sn-glycero-3-phosphocholine; 1-(1Z,9Z-octadecadienyl)-2-(9Z-hexadecenoyl)-sn-glycero-3-phosphocholine; 1-(1Z,9Z-octadecadienyl)-2-octadecanoyl-sn-glycero-3-phosphocholine; 1-(1Z,9Z-octadecadienyl)-2-(13Z,16Z-docosadienoyl)-sn-glycero-3-phosphocholine; (2R)-2-hydroxy-3-[(9Z,12Z)-nonadeca-9,12-dienoyloxy]propyl 2-(trimethylammonio)ethyl phosphate; 1-(11Z-octadecadienoyl)-sn-glycero-3-phosphocholine; 1-(5Z,8Z,11Z-eicosatrienoyl)-sn-glycero-3-phosphocholine; 1-(13Z-docosenoyl)-sn-glycero-3-phosphocholine; 1-(14Z,17Z,20Z,23Z,26Z,29Z-dotriacontahexaenoyl)-2-(4Z,7Z,10Z,13Z,16Z,19Z-docosahexaenoyl)-glycero-3-phosphocholine; 1-O-(2-methoxyhexadecyl)-sn-glycerol-3-phosphocholine; 1-O-(2-methoxy-4Z-hexadecenyl)-sn-glycero-3-phosphocholine; 1-undecanoyl-2-hexadecanoyl-sn-glycero-3-phosphocholine; 1-hexadecyl-2-(9-carboxy-nonanoyl)-sn-glycero-3-phosphocholine; 1-(15-methyl-)hexadecyl-sn-glycero-3-phosphocholine; 1-(3Z-octadecenyl-sn-glycero-3-phosphocholine; 1-(cis-11,12-methylene-octadecanoyl)-sn-glycero-3-phosphocholine; 1-(3Z-hexadecenyl-sn-glycero-3-phosphocholine; and/or 1-(4Z-hexadecenyl-sn-glycero-3-phosphocholine.

In some embodiments, the lipid may be a derivative of phosphatidylserine. Phosphatidylserines may be represented by formula (Ib):

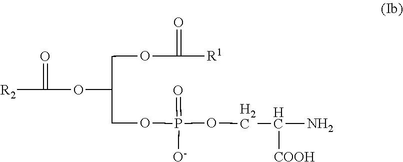

Non-limiting examples of derivatives of phosphatidylserine are 1-hexadecanoyl-2-(9Z-octadecenoyl)-sn-glycero-3-phosphoserine (POPS); 1,2-ditetradecanoyl-sn-glycero-3-phosphoserine (Dimyristoyl phosphatidylserine or DMPS); 1,2-di-(9Z-octadecenoyl)-sn-glycero-3-phosphoserine (Dioleoyl phosphatidylserine or DOPS); 1-dodecanoyl-2-tridecanoyl-sn-glycero-3-phosphoserine; 1-heptadecanoyl, 2-(5Z,8Z,11Z,14Z-eicosatetraenoyl)-sn-glycero-3-phosphoserine; 1-heneicosanoyl-2-(4Z,7Z,10Z,13Z,16Z,19Z-docosahexaenoyl)-sn-glycero-3-phosphoserine; 1-heptadecanoyl-2-(9Z-tetradecenoyl)-sn-glycero-3-phosphoserine; 1-hexadecanoyl-2-(11Z-octadecenoyl)-sn-glycero-3-phosphoserine; 1,2-dihexanoyl-sn-glycero-3-phosphoserine; 1,2-dioctanoyl-sn-glycero-3-phosphoserine; 1,2-didecanoyl-sn-glycero-3-phosphoserine; 1,2-di-(9Z,12Z-octadecadienoyl)-sn-glycero-3-phosphoserine; 1-octadecanoyl-2-(9Z-octadecenoyl)-sn-glycero-3-phosphoserine; 1-(9Z-octadecenoyl)-sn-glycero-3-phosphoserine; 1-hexadecanoyl-sn-glycero-3-phosphoserine; 1,2-didodecanoyl-sn-glycero-3-phosphoserine; 1,2-dihexadecanoyl-sn-glycero-3-phosphoserine; 1-octadecanoyl-2-(9Z,12Z-octadecadienoyl)-sn-glycero-3-phosphoserine; 1-(9Z-octadecenoyl)-2-octadecanoyl-sn-glycero-3-phosphoserine; 1-hexadecanoyl-2-eicosanoyl-sn-glycero-3-phosphoserine; 1,2-dioctadecanoyl-sn-glycero-3-phosphoserine; 1,2-diacyl-sn-glycero-3-phosphoserine; 2-acyl-sn-glycero-3-phosphoserine; 1-hexadecanoyl-2-(5Z,8Z,11 Z,14Z-eicosatetraenoyl)-sn-glycero-3-phosphoserine; 1-octadecanoyl-2-(5Z,8Z,11Z,14Z-eicosatetraenoyl)-sn-glycero-3-phosphoserine; 1-octadecanoyl-2-(4Z,7Z,10Z,13Z,16Z,19Z-docosahexaenoyl)-sn-glycero-3-phosphoserine; 1-octadecanoyl-2-(9Z-octadecenoyl)-sn-glycero-3-phospho-N-hexadecanoyl-L-serine; 1,2-di-(9Z-octadecenoyl)-sn-glycero-3-phospho-N-nonadecanoyl-L-serine; 1-tridecanoyl-sn-glycero-3-phosphoserine; 1-octadecanoyl-sn-glycero-3-phosphoserine; 1-(5Z,8Z,11Z,14Z-eicosatetraenoyl)-sn-glycero-3-phosphoserine; 1-hexadecanoyl-2-(4Z,7Z,10Z,13Z,16Z,19Z-docosahexaenoyl)-sn-glycero-3-phosphoserine; 1-(9Z-hexadecenoyl)-2-(4Z,7Z,10Z,13Z,16Z,19Z-docosahexaenoyl)-sn-glycero-3-phosphoserine; 1-(9Z-octadecenoyl)-2-(4Z,7Z,10Z,13Z,16Z,19Z-docosahexaenoyl)-sn-glycero-3-phosphoserine; 1,2-di-(4Z,7Z,10Z,13Z,16Z,19Z-docosahexaenoyl)-sn-glycero-3-phosphoserine; 1-dodecanoyl-glycero-3-phosphoserine; 1-tetradecanoyl-glycero-3-phosphoserine; 1-(9Z-hexadecenoyl)-glycero-3-phosphoserine; 1-(9Z,12Z-octadecadienoyl)-glycero-3-phosphoserine; 1-eicosanoyl-glycero-3-phosphoserine; 1-(4Z,7Z,10Z,13Z,16Z,19Z-docosahexaenoyl)-glycero-3-phosphoserine; 1-(7Z,10Z,13Z,16Z-docosatetraenoyl)-glycero-3-phosphoserine; 1-dodecanoyl-2-(9Z-tetradecenoyl)-glycero-3-phosphoserine; 1-dodecanoyl-2-pentadecanoyl-glycero-3-phosphoserine; 1-dodecanoyl-2-(9Z-pentadecenoyl)-glycero-3-phosphoserine; 1-dodecanoyl-2-(9Z-hexadecenoyl)-glycero-3-phosphoserine; 1-dodecanoyl-2-heptadecanoyl-glycero-3-phosphoserine; 1-dodecanoyl-2-(9Z-heptadecenoyl)-glycero-3-phosphoserine; 1-dodecanoyl-2-(9Z,12Z-heptadecadienoyl)-glycero-3-phosphoserine; 1-dodecanoyl-2-(9Z,12Z-octadecadienoyl)-glycero-3-phosphoserine; 1-dodecanoyl-2-(6Z,9Z,12Z-octadecatrienoyl)-glycero-3-phosphoserine; 1-dodecanoyl-2-(9Z,12Z,15Z-octadecatrienoyl)-glycero-3-phosphoserine; 1-dodecanoyl-2-(6Z,9Z,12Z,15Z-octadecatetraenoyl)-glycero-3-phosphoserine; 1-dodecanoyl-2-nonadecanoyl-glycero-3-phosphoserine; 1-dodecanoyl-2-(9Z-nonadecenoyl)-glycero-3-phosphoserine; 1-dodecanoyl-2-(11Z-eicosenoyl)-glycero-3-phosphoserine; 1-dodecanoyl-2-(11Z,14Z-eicosadienoyl)-glycero-3-phosphoserine; 1-dodecanoyl-2-(8Z,11Z,14Z-eicosatrienoyl)-glycero-3-phosphoserine; 1-dodecanoyl-2-(5Z,8Z,11Z,14Z-eicosatetraenoyl)-glycero-3-phosphoserine; 1-dodecanoyl-2-(5Z,8Z,11Z,14Z,17Z-eicosapentaenoyl)-glycero-3-phosphoserine; 1-dodecanoyl-2-heneicosanoyl-glycero-3-phosphoserine; 1-dodecanoyl-2-(11Z-docosenoyl)-glycero-3-phosphoserine; 1-dodecanoyl-2-(13Z,16Z-docosadienoyl)-glycero-3-phosphoserine; 1-dodecanoyl-2-(7Z,10Z,13Z,16Z-docosatetraenoyl)-glycero-3-phosphoserine; 1-tridecanoyl-2-dodecanoyl-glycero-3-phosphoserine; 1-tridecanoyl-2-tetradecanoyl-glycero-3-phosphoserine; 1-tridecanoyl-2-(9Z-tetradecenoyl)-glycero-3-phosphoserine; 1-tridecanoyl-2-(9Z-pentadecenoyl)-glycero-3-phosphoserine; 1-tridecanoyl-2-hexadecanoyl-glycero-3-phosphoserine; 1-tridecanoyl-2-(9Z-hexadecenoyl)-glycero-3-phosphoserine; 1-tridecanoyl-2-(9Z-heptadecenoyl)-glycero-3-phosphoserine; 1-tridecanoyl-2-(9Z,12Z-heptadecadienoyl)-glycero-3-phosphoserine; 1-tridecanoyl-2-octadecanoyl-glycero-3-phosphoserine; 1-tridecanoyl-2-(9Z-octadecenoyl)-glycero-3-phosphoserine; 1-tridecanoyl-2-(9Z,12Z-octadecadienoyl)-glycero-3-phosphoserine; 1-tridecanoyl-2-(6Z,9Z,12Z-octadecatrienoyl)-glycero-3-phosphoserine; 1-tridecanoyl-2-(9Z,12Z,15Z-octadecatrienoyl)-glycero-3-phosphoserine; 1-tridecanoyl-2-(6Z,9Z,12Z,15Z-octadecatetraenoyl)-glycero-3-phosphoserine; 1-tridecanoyl-2-(9Z-nonadecenoyl)-glycero-3-phosphoserine; 1-tridecanoyl-2-(11Z-eicosenoyl)-glycero-3-phosphoserine; 1-tridecanoyl-2-(11Z,14Z-eicosadienoyl)-glycero-3-phosphoserine; 1-tridecanoyl-2-(8Z,11Z,14Z-eicosatrienoyl)-glycero-3-phosphoserine; 1-tridecanoyl-2-(5Z,8Z,11Z,14Z-eicosatetraenoyl)-glycero-3-phosphoserine; 1-tridecanoyl-2-(5Z,8Z,11Z,14Z,17Z-eicosapentaenoyl)-glycero-3-phosphoserine; 1-tridecanoyl-2-docosanoyl-glycero-3-phosphoserine; 1-tridecanoyl-2-(11Z-docosenoyl)-glycero-3-phosphoserine; 1-tridecanoyl-2-(13Z,16Z-docosadienoyl)-glycero-3-phosphoserine; 1-tridecanoyl-2-(7Z,10Z,13Z,16Z-docosatetraenoyl)-glycero-3-phosphoserine; 1-tridecanoyl-2-(4Z,7Z,10Z,13Z,16Z,19Z-docosahexaenoyl)-glycero-3-phosphoserine; 1-tetradecanoyl-2-tridecanoyl-glycero-3-phosphoserine; 1-tetradecanoyl-2-(9Z-tetradecenoyl)-glycero-3-phosphoserine; 1-tetradecanoyl-2-(9Z-pentadecenoyl)-glycero-3-phosphoserine; 1-tetradecanoyl-2-heptadecanoyl-glycero-3-phosphoserine; 1-tetradecanoyl-2-(9Z-heptadecenoyl)-glycero-3-phosphoserine; 1-tetradecanoyl-2-(9Z,12Z-heptadecadienoyl)-glycero-3-phosphoserine; 1-tetradecanoyl-2-(6Z,9Z,12Z-octadecatrienoyl)-glycero-3-phosphoserine; 1-tetradecanoyl-2-nonadecanoyl-glycero-3-phosphoserine; 1-tetradecanoyl-2-(9Z-nonadecenoyl)-glycero-3-phosphoserine; 1-tetradecanoyl-2-(11Z-eicosenoyl)-glycero-3-phosphoserine; 1-tetradecanoyl-2-(11Z,14Z-eicosadienoyl)-glycero-3-phosphoserine; 1-tetradecanoyl-2-(8Z,11Z,14Z-eicosatrienoyl)-glycero-3-phosphoserine; 1-tetradecanoyl-2-heneicosanoyl-glycero-3-phosphoserine; 1-tetradecanoyl-2-(11Z-docosenoyl)-glycero-3-phosphoserine; 1-tetradecanoyl-2-(13Z,16Z-docosadienoyl)-glycero-3-phosphoserine; 1-tetradecanoyl-2-(7Z,10Z,13Z,16Z-docosatetraenoyl)-glycero-3-phosphoserine; 1-(9Z-tetradecenoyl)-2-dodecanoyl-glycero-3-phosphoserine; 1-(9Z-tetradecenoyl)-2-tridecanoyl-glycero-3-phosphoserine; 1-(9Z-tetradecenoyl)-2-tetradecanoyl-glycero-3-phosphoserine; 1-(9Z-tetradecenoyl)-2-pentadecanoyl-glycero-3-phosphoserine; 1-(9Z-tetradecenoyl)-2-(9Z-pentadecenoyl)-glycero-3-phosphoserine; 1-(9Z-tetradecenoyl)-2-hexadecanoyl-glycero-3-phosphoserine; 1-(9Z-tetradecenoyl)-2-(9Z-hexadecenoyl)-glycero-3-phosphoserine; 1-(9Z-tetradecenoyl)-2-heptadecanoyl-glycero-3-phosphoserine; 1-(9Z-tetradecenoyl)-2-(9Z-heptadecenoyl)-glycero-3-phosphoserine; 1-(9Z-tetradecenoyl)-2-(9Z,12Z-heptadecadienoyl)-glycero-3-phosphoserine; 1-(9Z-tetradecenoyl)-2-octadecanoyl-glycero-3-phosphoserine; 1-(9Z-tetradecenoyl)-2-(9Z-octadecenoyl)-glycero-3-phosphoserine; 1-(9Z-tetradecenoyl)-2-(9Z,12Z-octadecadienoyl)-glycero-3-phosphoserine; 1-(9Z-tetradecenoyl)-2-(6Z,9Z,12Z-octadecatrienoyl)-glycero-3-phosphoserine; 1-(9Z-tetradecenoyl)-2-(9Z,12Z,15Z-octadecatrienoyl)-glycero-3-phosphoserine; 1-(9Z-tetradecenoyl)-2-(6Z,9Z,12Z,15Z-octadecatetraenoyl)-glycero-3-phosphoserine; 1-(9Z-tetradecenoyl)-2-nonadecanoyl-glycero-3-phosphoserine; 1-(9Z-tetradecenoyl)-2-(9Z-nonadecenoyl)-glycero-3-phosphoserine; 1-(9Z-tetradecenoyl)-2-eicosanoyl-glycero-3-phosphoserine; 1-(9Z-tetradecenoyl)-2-(11Z-eicosenoyl)-glycero-3-phosphoserine; 1-(9Z-tetradecenoyl)-2-(11Z,14Z-eicosadienoyl)-glycero-3-phosphoserine; 1-(9Z-tetradecenoyl)-2-(8Z,11Z,14Z-eicosatrienoyl)-glycero-3-phosphoserine; 1-(9Z-tetradecenoyl)-2-(5Z,8Z,11Z,14Z-eicosatetraenoyl)-glycero-3-phosphoserine; 1-(9Z-tetradecenoyl)-2-(5Z,8Z,11Z,14Z,17Z-eicosapentaenoyl)-glycero-3-phosphoserine; 1-(9Z-tetradecenoyl)-2-heneicosanoyl-glycero-3-phosphoserine; 1-(9Z-tetradecenoyl)-2-docosanoyl-glycero-3-phosphoserine; 1-(9Z-tetradecenoyl)-2-(11 Z-docosenoyl)-glycero-3-phosphoserine; 1-(9Z-tetradecenoyl)-2-(13Z,16Z-docosadienoyl)-glycero-3-phosphoserine; 1-(9Z-tetradecenoyl)-2-(7Z,10Z,13Z,16Z-docosatetraenoyl)-glycero-3-phosphoserine; 1-(9Z-tetradecenoyl)-2-(4Z,7Z,10Z,13Z,16Z,19Z-docosahexaenoyl)-glycero-3-phosphoserine; 1-pentadecanoyl-2-dodecanoyl-glycero-3-phosphoserine; 1-pentadecanoyl-2-tetradecanoyl-glycero-3-phosphoserine; 1-pentadecanoyl-2-(9Z-tetradecenoyl)-glycero-3-phosphoserine; 1-pentadecanoyl-2-(9Z-pentadecenoyl)-glycero-3-phosphoserine; 1-pentadecanoyl-2-(9Z-heptadecenoyl)-glycero-3-phosphoserine; 1-pentadecanoyl-2-(9Z,12Z-heptadecadienoyl)-glycero-3-phosphoserine; 1-pentadecanoyl-2-(9Z-octadecenoyl)-glycero-3-phosphoserine; 1-pentadecanoyl-2-(6Z,9Z,12Z-octadecatrienoyl)-glycero-3-phosphoserine; 1-pentadecanoyl-2-(9Z,12Z,15Z-octadecatrienoyl)-glycero-3-phosphoserine; 1-pentadecanoyl-2-(6Z,9Z,12Z,15Z-octadecatetraenoyl)-glycero-3-phosphoserine; 1-pentadecanoyl-2-(9Z-nonadecenoyl)-glycero-3-phosphoserine; 1-pentadecanoyl-2-eicosanoyl-glycero-3-phosphoserine; 1-pentadecanoyl-2-(11Z-eicosenoyl)-glycero-3-phosphoserine; 1-pentadecanoyl-2-(11Z,14Z-eicosadienoyl)-glycero-3-phosphoserine; 1-pentadecanoyl-2-(8Z,11Z,14Z-eicosatrienoyl)-glycero-3-phosphoserine; 1-pentadecanoyl-2-(5Z,8Z,11Z,14Z-eicosatetraenoyl)-glycero-3-phosphoserine; 1-pentadecanoyl-2-(5Z,8Z,11Z,14Z,17Z-eicosapentaenoyl)-glycero-3-phosphoserine; 1-pentadecanoyl-2-docosanoyl-glycero-3-phosphoserine; 1-pentadecanoyl-2-(11Z-docosenoyl)-glycero-3-phosphoserine; 1-pentadecanoyl-2-(13Z,16Z-docosadienoyl)-glycero-3-phosphoserine; 1-pentadecanoyl-2-(7Z,10Z,13Z,16Z-docosatetraenoyl)-glycero-3-phosphoserine; 1-pentadecanoyl-2-(4Z,7Z,10Z,13Z,16Z,19Z-docosahexaenoyl)-glycero-3-phosphoserine; 1-(9Z-pentadecenoyl)-glycero-3-phosphoserine; 1-(9Z-pentadecenoyl)-2-dodecanoyl-glycero-3-phosphoserine; 1-(9Z-pentadecenoyl)-2-tridecanoyl-glycero-3-phosphoserine; 1-(9Z-pentadecenoyl)-2-tetradecanoyl-glycero-3-phosphoserine; 1-(9Z-pentadecenoyl)-2-(9Z-tetradecenoyl)-glycero-3-phosphoserine; 1-(9Z-pentadecenoyl)-2-pentadecanoyl-glycero-3-phosphoserine; 1,2-di-(9Z-pentadecenoyl)-sn-glycero-3-phosphoserine; 1-(9Z-pentadecenoyl)-2-hexadecanoyl-glycero-3-phosphoserine; 1-(9Z-pentadecenoyl)-2-(9Z-hexadecenoyl)-glycero-3-phosphoserine; 1-(9Z-pentadecenoyl)-2-heptadecanoyl-glycero-3-phosphoserine; 1-(9Z-pentadecenoyl)-2-(9Z-heptadecenoyl)-glycero-3-phosphoserine; 1-(9Z-pentadecenoyl)-2-(9Z,12Z-heptadecadienoyl)-glycero-3-phosphoserine; 1-(9Z-pentadecenoyl)-2-octadecanoyl-glycero-3-phosphoserine; 1-(9Z-pentadecenoyl)-2-(9Z-octadecenoyl)-glycero-3-phosphoserine; 1-(9Z-pentadecenoyl)-2-(9Z,12Z-octadecadienoyl)-glycero-3-phosphoserine; 1-(9Z-pentadecenoyl)-2-(6Z,9Z,12Z-octadecatrienoyl)-glycero-3-phosphoserine; 1-(9Z-pentadecenoyl)-2-(9Z,12Z,15Z-octadecatrienoyl)-glycero-3-phosphoserine; 1-(9Z-pentadecenoyl)-2-(6Z,9Z,12Z,15Z-octadecatetraenoyl)-glycero-3-phosphoserine; 1-(9Z-pentadecenoyl)-2-nonadecanoyl-glycero-3-phosphoserine; 1-(9Z-pentadecenoyl)-2-(9Z-nonadecenoyl)-glycero-3-phosphoserine; 1-(9Z-pentadecenoyl)-2-eicosanoyl-glycero-3-phosphoserine; 1-(9Z-pentadecenoyl)-2-(11 Z-eicosenoyl)-glycero-3-phosphoserine; 1-(9Z-pentadecenoyl)-2-(11Z,14Z-eicosadienoyl)-glycero-3-phosphoserine; 1-(9Z-pentadecenoyl)-2-(8Z,11Z,14Z-eicosatrienoyl)-glycero-3-phosphoserine; 1-(9Z-pentadecenoyl)-2-(5Z,8Z,11Z,14Z-eicosatetraenoyl)-glycero-3-phosphoserine; 1-(9Z-pentadecenoyl)-2-(5Z,8Z,11Z,14Z,17Z-eicosapentaenoyl)-glycero-3-phosphoserine; 1-(9Z-pentadecenoyl)-2-heneicosanoyl-glycero-3-phosphoserine; 1-(9Z-pentadecenoyl)-2-docosanoyl-glycero-3-phosphoserine; 1-(9Z-pentadecenoyl)-2-(11Z-docosenoyl)-glycero-3-phosphoserine; 1-(9Z-pentadecenoyl)-2-(13Z,16Z-docosadienoyl)-glycero-3-phosphoserine; 1-(9Z-pentadecenoyl)-2-(7Z,10Z,13Z,16Z-docosatetraenoyl)-glycero-3-phosphoserine; 1-(9Z-pentadecenoyl)-2-(4Z,7Z,10Z,13Z,16Z,19Z-docosahexaenoyl)-glycero-3-phosphoserine; 1-hexadecanoyl-2-tridecanoyl-glycero-3-phosphoserine; 1-hexadecanoyl-2-(9Z-tetradecenoyl)-glycero-3-phosphoserine; 1-hexadecanoyl-2-(9Z-pentadecenoyl)-glycero-3-phosphoserine; 1-hexadecanoyl-2-(9Z,12Z-heptadecadienoyl)-glycero-3-phosphoserine; 1-hexadecanoyl-2-(9Z-nonadecenoyl)-glycero-3-phosphoserine; 1-hexadecanoyl-2-(11Z,14Z-eicosadienoyl)-glycero-3-phosphoserine; 1-hexadecanoyl-2-heneicosanoyl-glycero-3-phosphoserine; 1-hexadecanoyl-2-(11Z-docosenoyl)-glycero-3-phosphoserine; 1-hexadecanoyl-2-(13Z,16Z-docosadienoyl)-glycero-3-phosphoserine; 1-(9Z-hexadecenoyl)-2-dodecanoyl-glycero-3-phosphoserine; 1-(9Z-hexadecenoyl)-2-tridecanoyl-glycero-3-phosphoserine; 1-(9Z-hexadecenoyl)-2-tetradecanoyl-glycero-3-phosphoserine; 1-(9Z-hexadecenoyl)-2-(9Z-tetradecenoyl)-glycero-3-phosphoserine; 1-(9Z-hexadecenoyl)-2-pentadecanoyl-glycero-3-phosphoserine; 1-(9Z-hexadecenoyl)-2-(9Z-pentadecenoyl)-glycero-3-phosphoserine; 1-(9Z-hexadecenoyl)-2-hexadecanoyl-glycero-3-phosphoserine; 1-(9Z-hexadecenoyl)-2-heptadecanoyl-glycero-3-phosphoserine; 1-(9Z-hexadecenoyl)-2-(9Z-heptadecenoyl)-glycero-3-phosphoserine; 1-(9Z-hexadecenoyl)-2-(9Z,12Z-heptadecadienoyl)-glycero-3-phosphoserine; 1-(9Z-hexadecenoyl)-2-octadecanoyl-glycero-3-phosphoserine; 1-(9Z-hexadecenoyl)-2-(6Z,9Z,12Z-octadecatrienoyl)-glycero-3-phosphoserine; 1-(9Z-hexadecenoyl)-2-(9Z,12Z,15Z-octadecatrienoyl)-glycero-3-phosphoserine; 1-(9Z-hexadecenoyl)-2-nonadecanoyl-glycero-3-phosphoserine; 1-(9Z-hexadecenoyl)-2-(9Z-nonadecenoyl)-glycero-3-phosphoserine; 1-(9Z-hexadecenoyl)-2-eicosanoyl-glycero-3-phosphoserine; 1-(9Z-hexadecenoyl)-2-(11Z-eicosenoyl)-glycero-3-phosphoserine; 1-(9Z-hexadecenoyl)-2-(11Z,14Z-eicosadienoyl)-glycero-3-phosphoserine; 1-(9Z-hexadecenoyl)-2-(8Z,11Z,14Z-eicosatrienoyl)-glycero-3-phosphoserine; 1-(9Z-hexadecenoyl)-2-(5Z,8Z,11Z,14Z,17Z-eicosapentaenoyl)-glycero-3-phosphoserine; 1-(9Z-hexadecenoyl)-2-heneicosanoyl-glycero-3-phosphoserine; 1-(9Z-hexadecenoyl)-2-docosanoyl-glycero-3-phosphoserine; 1-(9Z-hexadecenoyl)-2-(11Z-docosenoyl)-glycero-3-phosphoserine; 1-(9Z-hexadecenoyl)-2-(13Z,16Z-docosadienoyl)-glycero-3-phosphoserine; 1-(9Z-hexadecenoyl)-2-(7Z,10Z,13Z,16Z-docosatetraenoyl)-glycero-3-phosphoserine; 1-heptadecanoyl-2-dodecanoyl-glycero-3-phosphoserine; 1-heptadecanoyl-2-tetradecanoyl-glycero-3-phosphoserine; 1-heptadecanoyl-2-(9Z-pentadecenoyl)-glycero-3-phosphoserine; 1-heptadecanoyl-2-hexadecanoyl-glycero-3-phosphoserine; 1-heptadecanoyl-2-(9Z-hexadecenoyl)-glycero-3-phosphoserine; 1-heptadecanoyl-2-(9Z-heptadecenoyl)-glycero-3-phosphoserine; 1-heptadecanoyl-2-(9Z,12Z-heptadecadienoyl)-glycero-3-phosphoserine; 1-heptadecanoyl-2-(9Z,12Z-octadecadienoyl)-glycero-3-phosphoserine; 1-heptadecanoyl-2-(6Z,9Z,12Z-octadecatrienoyl)-glycero-3-phosphoserine; 1-heptadecanoyl-2-(9Z,12Z,15Z-octadecatrienoyl)-glycero-3-phosphoserine; 1-heptadecanoyl-2-(6Z,9Z,12Z,15Z-octadecatetraenoyl)-glycero-3-phosphoserine; 1-heptadecanoyl-2-nonadecanoyl-glycero-3-phosphoserine; 1-heptadecanoyl-2-(9Z-nonadecenoyl)-glycero-3-phosphoserine; 1-heptadecanoyl-2-(11Z-eicosenoyl)-glycero-3-phosphoserine; 1-heptadecanoyl-2-(11Z,14Z-eicosadienoyl)-glycero-3-phosphoserine; 1-heptadecanoyl-2-(8Z,11Z,14Z-eicosatrienoyl)-glycero-3-phosphoserine; 1-heptadecanoyl-2-(5Z,8Z,11Z,14Z,17Z-eicosapentaenoyl)-glycero-3-phosphoserine; 1-heptadecanoyl-2-heneicosanoyl-glycero-3-phosphoserine; 1-heptadecanoyl-2-docosanoyl-glycero-3-phosphoserine; 1-heptadecanoyl-2-(11Z-docosenoyl)-glycero-3-phosphoserine; 1-heptadecanoyl-2-(13Z,16Z-docosadienoyl)-glycero-3-phosphoserine; 1-heptadecanoyl-2-(7Z,10Z,13Z,16Z-docosatetraenoyl)-glycero-3-phosphoserine; 1-(9Z-heptadecenoyl)-2-dodecanoyl-glycero-3-phosphoserine; 1-(9Z-heptadecenoyl)-2-tridecanoyl-glycero-3-phosphoserine; 1-(9Z-heptadecenoyl)-2-tetradecanoyl-glycero-3-phosphoserine; 1-(9Z-heptadecenoyl)-2-(9Z-tetradecenoyl)-glycero-3-phosphoserine; 1-(9Z-heptadecenoyl)-2-pentadecanoyl-glycero-3-phosphoserine; 1-(9Z-heptadecenoyl)-2-(9Z-pentadecenoyl)-glycero-3-phosphoserine; 1-(9Z-heptadecenoyl)-2-hexadecanoyl-glycero-3-phosphoserine; 1-(9Z-heptadecenoyl)-2-(9Z-hexadecenoyl)-glycero-3-phosphoserine; 1-(9Z-heptadecenoyl)-2-heptadecanoyl-glycero-3-phosphoserine; 1-(9Z-heptadecenoyl)-2-(9Z,12Z-heptadecadienoyl)-glycero-3-phosphoserine; 1-(9Z-heptadecenoyl)-2-octadecanoyl-glycero-3-phosphoserine; 1-(9Z-heptadecenoyl)-2-(9Z-octadecenoyl)-glycero-3-phosphoserine; 1-(9Z-heptadecenoyl)-2-(9Z,12Z-octadecadienoyl)-glycero-3-phosphoserine; 1-(9Z-heptadecenoyl)-2-(6Z,9Z,12Z-octadecatrienoyl)-glycero-3-phosphoserine; 1-(9Z-heptadecenoyl)-2-(9Z,12Z,15Z-octadecatrienoyl)-glycero-3-phosphoserine; 1-(9Z-heptadecenoyl)-2-(6Z,9Z,12Z,15Z-octadecatetraenoyl)-glycero-3-phosphoserine; 1-(9Z-heptadecenoyl)-2-nonadecanoyl-glycero-3-phosphoserine; 1-(9Z-heptadecenoyl)-2-(9Z-nonadecenoyl)-glycero-3-phosphoserine; 1-(9Z-heptadecenoyl)-2-eicosanoyl-glycero-3-phosphoserine; 1-(9Z-heptadecenoyl)-2-(11 Z-eicosenoyl)-glycero-3-phosphoserine; 1-(9Z-heptadecenoyl)-2-(11Z,14Z-eicosadienoyl)-glycero-3-phosphoserine; 1-(9Z-heptadecenoyl)-2-(8Z,11Z,14Z-eicosatrienoyl)-glycero-3-phosphoserine; 1-(9Z-heptadecenoyl)-2-(5Z,8Z,11Z,14Z-eicosatetraenoyl)-glycero-3-phosphoserine; 1-(9Z-heptadecenoyl)-2-(5Z,8Z,11Z,14Z,17Z-eicosapentaenoyl)-glycero-3-phosphoserine; 1-(9Z-heptadecenoyl)-2-heneicosanoyl-glycero-3-phosphoserine; 1-(9Z-heptadecenoyl)-2-docosanoyl-glycero-3-phosphoserine; 1-(9Z-heptadecenoyl)-2-(11Z-docosenoyl)-glycero-3-phosphoserine; 1-(9Z-heptadecenoyl)-2-(13Z,16Z-docosadienoyl)-glycero-3-phosphoserine; 1-(9Z-heptadecenoyl)-2-(7Z,10Z,13Z,16Z-docosatetraenoyl)-glycero-3-phosphoserine; 1-(9Z-heptadecenoyl)-2-(4Z,7Z,10Z,13Z,16Z,19Z-docosahexaenoyl)-glycero-3-phosphoserine; 1-(9Z,12Z-heptadecadienoyl)-glycero-3-phosphoserine; 1-(9Z,12Z-heptadecadienoyl)-2-dodecanoyl-glycero-3-phosphoserine; 1-(9Z,12Z-heptadecadienoyl)-2-tridecanoyl-glycero-3-phosphoserine; 1-(9Z,12Z-heptadecadienoyl)-2-tetradecanoyl-glycero-3-phosphoserine; 1-(9Z,12Z-heptadecadienoyl)-2-(9Z-tetradecenoyl)-glycero-3-phosphoserine; 1-(9Z,12Z-heptadecadienoyl)-2-pentadecanoyl-glycero-3-phosphoserine; 1-(9Z,12Z-heptadecadienoyl)-2-(9Z-pentadecenoyl)-glycero-3-phosphoserine; 1-(9Z,12Z-heptadecadienoyl)-2-hexadecanoyl-glycero-3-phosphoserine; 1-(9Z,12Z-heptadecadienoyl)-2-(9Z-hexadecenoyl)-glycero-3-phosphoserine; 1-(9Z,12Z-heptadecadienoyl)-2-heptadecanoyl-glycero-3-phosphoserine; 1-(9Z,12Z-heptadecadienoyl)-2-(9Z-heptadecenoyl)-glycero-3-phosphoserine; 1-(9Z,12Z-heptadecadienoyl)-2-octadecanoyl-glycero-3-phosphoserine; 1-(9Z,12Z-heptadecadienoyl)-2-(9Z-octadecenoyl)-glycero-3-phosphoserine; 1-(9Z,12Z-heptadecadienoyl)-2-(9Z,12Z-octadecadienoyl)-glycero-3-phosphoserine; 1-(9Z,12Z-heptadecadienoyl)-2-(6Z,9Z,12Z-octadecatrienoyl)-glycero-3-phosphoserine; 1-(9Z,12Z-heptadecadienoyl)-2-(9Z,12Z,15Z-octadecatrienoyl)-glycero-3-phosphoserine; 1-(9Z,12Z-heptadecadienoyl)-2-(6Z,9Z,12Z,15Z-octadecatetraenoyl)-glycero-3-phosphoserine; 1-(9Z,12Z-heptadecadienoyl)-2-nonadecanoyl-glycero-3-phosphoserine; 1-(9Z,12Z-heptadecadienoyl)-2-(9Z-nonadecenoyl)-glycero-3-phosphoserine; 1-(9Z,12Z-heptadecadienoyl)-2-eicosanoyl-glycero-3-phosphoserine; 1-(9Z,12Z-heptadecadienoyl)-2-(11Z-eicosenoyl)-glycero-3-phosphoserine; 1-(9Z,12Z-heptadecadienoyl)-2-(11Z,14Z-eicosadienoyl)-glycero-3-phosphoserine; 1-(9Z,12Z-heptadecadienoyl)-2-(8Z,11Z,14Z-eicosatrienoyl)-glycero-3-phosphoserine; 1-(9Z,12Z-heptadecadienoyl)-2-(5Z,8Z,11Z,14Z-eicosatetraenoyl)-glycero-3-phosphoserine; 1-(9Z,12Z-heptadecadienoyl)-2-(5Z,8Z,11Z,14Z,17Z-eicosapentaenoyl)-glycero-3-phosphoserine; 1-(9Z,12Z-heptadecadienoyl)-2-heneicosanoyl-glycero-3-phosphoserine; 1-(9Z,12Z-heptadecadienoyl)-2-docosanoyl-glycero-3-phosphoserine; 1-(9Z,12Z-heptadecadienoyl)-2-(11Z-docosenoyl)-glycero-3-phosphoserine; 1-(9Z,12Z-heptadecadienoyl)-2-(13Z,16Z-docosadienoyl)-glycero-3-phosphoserine; 1-(9Z,12Z-heptadecadienoyl)-2-(7Z,10Z,13Z,16Z-docosatetraenoyl)-glycero-3-phosphoserine; 1-(9Z,12Z-heptadecadienoyl)-2-(4Z,7Z,10Z,13Z,16Z,19Z-docosahexaenoyl)-glycero-3-phosphoserine; 1-octadecanoyl-2-tridecanoyl-glycero-3-phosphoserine; 1-octadecanoyl-2-(9Z-tetradecenoyl)-glycero-3-phosphoserine; 1-octadecanoyl-2-pentadecanoyl-glycero-3-phosphoserine; 1-octadecanoyl-2-(9Z-pentadecenoyl)-glycero-3-phosphoserine; 1-octadecanoyl-2-heptadecanoyl-glycero-3-phosphoserine; 1-octadecanoyl-2-(9Z-heptadecenoyl)-glycero-3-phosphoserine; 1-octadecanoyl-2-(9Z,12Z-heptadecadienoyl)-glycero-3-phosphoserine; 1-octadecanoyl-2-(6Z,9Z,12Z-octadecatrienoyl)-glycero-3-phosphoserine; 1-octadecanoyl-2-(9Z,12Z,15Z-octadecatrienoyl)-glycero-3-phosphoserine; 1-octadecanoyl-2-nonadecanoyl-glycero-3-phosphoserine; 1-octadecanoyl-2-(9Z-nonadecenoyl)-glycero-3-phosphoserine; 1-octadecanoyl-2-heneicosanoyl-glycero-3-phosphoserine; 1-octadecanoyl-2-(11Z-docosenoyl)-glycero-3-phosphoserine; 1-octadecanoyl-2-(13Z,16Z-docosadienoyl)-glycero-3-phosphoserine; 1-(9Z-octadecenoyl)-2-dodecanoyl-glycero-3-phosphoserine; 1-(9Z-octadecenoyl)-2-tridecanoyl-glycero-3-phosphoserine; 1-(9Z-octadecenoyl)-2-(9Z-tetradecenoyl)-glycero-3-phosphoserine; 1-(9Z-octadecenoyl)-2-pentadecanoyl-glycero-3-phosphoserine; 1-(9Z-octadecenoyl)-2-(9Z-pentadecenoyl)-glycero-3-phosphoserine; 1-(9Z-octadecenoyl)-2-heptadecanoyl-glycero-3-phosphoserine; 1-(9Z-octadecenoyl)-2-(9Z-heptadecenoyl)-glycero-3-phosphoserine; 1-(9Z-octadecenoyl)-2-(9Z,12Z-heptadecadienoyl)-glycero-3-phosphoserine; 1-(9Z-octadecenoyl)-2-(6Z,9Z,12Z-octadecatrienoyl)-glycero-3-phosphoserine; 1-(9Z-octadecenoyl)-2-(6Z,9Z,12Z,15Z-octadecatetraenoyl)-glycero-3-phosphoserine; 1-(9Z-octadecenoyl)-2-nonadecanoyl-glycero-3-phosphoserine; 1-(9Z-octadecenoyl)-2-(9Z-nonadecenoyl)-glycero-3-phosphoserine; 1-(9Z-octadecenoyl)-2-(11Z,14Z-eicosadienoyl)-glycero-3-phosphoserine; 1-(9Z-octadecenoyl)-2-(8Z,11Z,14Z-eicosatrienoyl)-glycero-3-phosphoserine; 1-(9Z-octadecenoyl)-2-heneicosanoyl-glycero-3-phosphoserine; 1-(9Z-octadecenoyl)-2-(11Z-docosenoyl)-glycero-3-phosphoserine; 1-(9Z-octadecenoyl)-2-(13Z,16Z-docosadienoyl)-glycero-3-phosphoserine; 1-(9Z-octadecenoyl)-2-(7Z,10Z,13Z,16Z-docosatetraenoyl)-glycero-3-phosphoserine; 1-(9Z,12Z-octadecadienoyl)-2-dodecanoyl-glycero-3-phosphoserine; 1-(9Z,12Z-octadecadienoyl)-2-tridecanoyl-glycero-3-phosphoserine; 1-(9Z,12Z-octadecadienoyl)-2-tetradecanoyl-glycero-3-phosphoserine; 1-(9Z,12Z-octadecadienoyl)-2-(9Z-tetradecenoyl)-glycero-3-phosphoserine; 1-(9Z,12Z-octadecadienoyl)-2-pentadecanoyl-glycero-3-phosphoserine; 1-(9Z,12Z-octadecadienoyl)-2-(9Z-pentadecenoyl)-glycero-3-phosphoserine; 1-(9Z,12Z-octadecadienoyl)-2-(9Z-hexadecenoyl)-glycero-3-phosphoserine; 1-(9Z,12Z-octadecadienoyl)-2-heptadecanoyl-glycero-3-phosphoserine; 1-(9Z,12Z-octadecadienoyl)-2-(9Z-heptadecenoyl)-glycero-3-phosphoserine; 1-(9Z,12Z-octadecadienoyl)-2-(9Z,12Z-heptadecadienoyl)-glycero-3-phosphoserine; 1-(9Z,12Z-octadecadienoyl)-2-(9Z-octadecenoyl)-glycero-3-phosphoserine; 1-(9Z,12Z-octadecadienoyl)-2-(6Z,9Z,12Z-octadecatrienoyl)-glycero-3-phosphoserine; 1-(9Z,12Z-octadecadienoyl)-2-(9Z,12Z,15Z-octadecatrienoyl)-glycero-3-phosphoserine; 1-(9Z,12Z-octadecadienoyl)-2-(6Z,9Z,12Z,15Z-octadecatetraenoyl)-glycero-3-phosphoserine; 1-(9Z,12Z-octadecadienoyl)-2-nonadecanoyl-glycero-3-phosphoserine; 1-(9Z,12Z-octadecadienoyl)-2-(9Z-nonadecenoyl)-glycero-3-phosphoserine; 1-(9Z,12Z-octadecadienoyl)-2-eicosanoyl-glycero-3-phosphoserine; 1-(9Z,12Z-octadecadienoyl)-2-(11Z-eicosenoyl)-glycero-3-phosphoserine; 1-(9Z,12Z-octadecadienoyl)-2-(11Z,14Z-eicosadienoyl)-glycero-3-phosphoserine; 1-(9Z,12Z-octadecadienoyl)-2-(8Z,11Z,14Z-eicosatrienoyl)-glycero-3-phosphoserine; 1-(9Z,12Z-octadecadienoyl)-2-(5Z,8Z,11Z,14Z,17Z-eicosapentaenoyl)-glycero-3-phosphoserine; 1-(9Z,12Z-octadecadienoyl)-2-heneicosanoyl-glycero-3-phosphoserine; 1-(9Z,12Z-octadecadienoyl)-2-docosanoyl-glycero-3-phosphoserine; 1-(9Z,12Z-octadecadienoyl)-2-(11Z-docosenoyl)-glycero-3-phosphoserine; 1-(9Z,12Z-octadecadienoyl)-2-(13Z,16Z-docosadienoyl)-glycero-3-phosphoserine; 1-(9Z,12Z-octadecadienoyl)-2-(7Z,10Z,13Z,16Z-docosatetraenoyl)-glycero-3-phosphoserine; 1-(6Z,9Z,12Z-octadecatrienoyl)-glycero-3-phosphoserine; 1-(6Z,9Z,12Z-octadecatrienoyl)-2-dodecanoyl-glycero-3-phosphoserine; 1-(6Z,9Z,12Z-octadecatrienoyl)-2-tridecanoyl-glycero-3-phosphoserine; 1-(6Z,9Z,12Z-octadecatrienoyl)-2-tetradecanoyl-glycero-3-phosphoserine; 1-(6Z,9Z,12Z-octadecatrienoyl)-2-(9Z-tetradecenoyl)-glycero-3-phosphoserine; 1-(6Z,9Z,12Z-octadecatrienoyl)-2-pentadecanoyl-glycero-3-phosphoserine; 1-(6Z,9Z,12Z-octadecatrienoyl)-2-(9Z-pentadecenoyl)-glycero-3-phosphoserine; 1-(6Z,9Z,12Z-octadecatrienoyl)-2-hexadecanoyl-glycero-3-phosphoserine; 1-(6Z,9Z,12Z-octadecatrienoyl)-2-(9Z-hexadecenoyl)-glycero-3-phosphoserine; 1-(6Z,9Z,12Z-octadecatrienoyl)-2-heptadecanoyl-glycero-3-phosphoserine; 1-(6Z,9Z,12Z-octadecatrienoyl)-2-(9Z-heptadecenoyl)-glycero-3-phosphoserine; 1-(6Z,9Z,12Z-octadecatrienoyl)-2-(9Z,12Z-heptadecadienoyl)-glycero-3-phosphoserine; 1-(6Z,9Z,12Z-octadecatrienoyl)-2-octadecanoyl-glycero-3-phosphoserine; 1-(6Z,9Z,12Z-octadecatrienoyl)-2-(9Z-octadecenoyl)-glycero-3-phosphoserine; 1-(6Z,9Z,12Z-octadecatrienoyl)-2-(9Z,12Z-octadecadienoyl)-glycero-3-phosphoserine; 1,2-di-(6Z,9Z,12Z-octadecatrienoyl)-sn-glycero-3-phosphoserine; 1-(6Z,9Z,12Z-octadecatrienoyl)-2-(9Z,12Z,15Z-octadecatrienoyl)-glycero-3-phosphoserine; 1-(6Z,9Z,12Z-octadecatrienoyl)-2-(6Z,9Z,12Z,15Z-octadecatetraenoyl)-glycero-3-phosphoserine; 1-(6Z,9Z,12Z-octadecatrienoyl)-2-nonadecanoyl-glycero-3-phosphoserine; 1-(6Z,9Z,12Z-octadecatrienoyl)-2-(9Z-nonadecenoyl)-glycero-3-phosphoserine; 1-(6Z,9Z,12Z-octadecatrienoyl)-2-eicosanoyl-glycero-3-phosphoserine; 1-(6Z,9Z,12Z-octadecatrienoyl)-2-(11 Z-eicosenoyl)-glycero-3-phosphoserine; 1-(6Z,9Z,12Z-octadecatrienoyl)-2-(11Z,14Z-eicosadienoyl)-glycero-3-phosphoserine; 1-(6Z,9Z,12Z-octadecatrienoyl)-2-(8Z,11Z,14Z-eicosatrienoyl)-glycero-3-phosphoserine; 1-(6Z,9Z,12Z-octadecatrienoyl)-2-(5Z,8Z,11Z,14Z-eicosatetraenoyl)-glycero-3-phosphoserine; 1-(6Z,9Z,12Z-octadecatrienoyl)-2-(5Z,8Z,11Z,14Z,17Z-eicosapentaenoyl)-glycero-3-phosphoserine; 1-(6Z,9Z,12Z-octadecatrienoyl)-2-heneicosanoyl-glycero-3-phosphoserine; 1-(6Z,9Z,12Z-octadecatrienoyl)-2-docosanoyl-glycero-3-phosphoserine; 1-(6Z,9Z,12Z-octadecatrienoyl)-2-(11Z-docosenoyl)-glycero-3-phosphoserine; 1-(6Z,9Z,12Z-octadecatrienoyl)-2-(13Z,16Z-docosadienoyl)-glycero-3-phosphoserine; 1-(6Z,9Z,12Z-octadecatrienoyl)-2-(7Z,10Z,13Z,16Z-docosatetraenoyl)-glycero-3-phosphoserine; 1-(6Z,9Z,12Z-octadecatrienoyl)-2-(4Z,7Z,10Z,13Z,16Z,19Z-docosahexaenoyl)-glycero-3-phosphoserine; 1-(9Z,12Z,15Z-octadecatrienoyl)-2-dodecanoyl-glycero-3-phosphoserine; 1-(9Z,12Z,15Z-octadecatrienoyl)-2-tridecanoyl-glycero-3-phosphoserine; 1-(9Z,12Z,15Z-octadecatrienoyl)-2-tetradecanoyl-glycero-3-phosphoserine; 1-(9Z,12Z,15Z-octadecatrienoyl)-2-(9Z-tetradecenoyl)-glycero-3-phosphoserine; 1-(9Z,12Z,15Z-octadecatrienoyl)-2-pentadecanoyl-glycero-3-phosphoserine; 1-(9Z,12Z,15Z-octadecatrienoyl)-2-(9Z-pentadecenoyl)-glycero-3-phosphoserine; 1-(9Z,12Z,15Z-octadecatrienoyl)-2-(9Z-hexadecenoyl)-glycero-3-phosphoserine; 1-(9Z,12Z,15Z-octadecatrienoyl)-2-heptadecanoyl-glycero-3-phosphoserine; 1-(9Z,12Z,15Z-octadecatrienoyl)-2-(9Z-heptadecenoyl)-glycero-3-phosphoserine; 1-(9Z,12Z,15Z-octadecatrienoyl)-2-(9Z,12Z-heptadecadienoyl)-glycero-3-phosphoserine; 1-(9Z,12Z,15Z-octadecatrienoyl)-2-octadecanoyl-glycero-3-phosphoserine; 1-(9Z,12Z,15Z-octadecatrienoyl)-2-(9Z,12Z-octadecadienoyl)-glycero-3-phosphoserine; 1-(9Z,12Z,15Z-octadecatrienoyl)-2-(6Z,9Z,12Z-octadecatrienoyl)-glycero-3-phosphoserine; 1-(9Z,12Z,15Z-octadecatrienoyl)-2-(6Z,9Z,12Z,15Z-octadecatetraenoyl)-glycero-3-phosphoserine; 1-(9Z,12Z,15Z-octadecatrienoyl)-2-nonadecanoyl-glycero-3-phosphoserine; 1-(9Z,12Z,15Z-octadecatrienoyl)-2-(9Z-nonadecenoyl)-glycero-3-phosphoserine; 1-(9Z,12Z,15Z-octadecatrienoyl)-2-eicosanoyl-glycero-3-phosphoserine; 1-(9Z,12Z,15Z-octadecatrienoyl)-2-(11Z-eicosenoyl)-glycero-3-phosphoserine; 1-(9Z,12Z,15Z-octadecatrienoyl)-2-(11Z,14Z-eicosadienoyl)-glycero-3-phosphoserine; 1-(9Z,12Z,15Z-octadecatrienoyl)-2-(8Z,11Z,14Z-eicosatrienoyl)-glycero-3-phosphoserine; 1-(9Z,12Z,15Z-octadecatrienoyl)-2-(5Z,8Z,11Z,14Z-eicosatetraenoyl)-glycero-3-phosphoserine; 1-(9Z,12Z,15Z-octadecatrienoyl)-2-(5Z,8Z,11Z,14Z,17Z-eicosapentaenoyl)-glycero-3-phosphoserine; 1-(9Z,12Z,15Z-octadecatrienoyl)-2-heneicosanoyl-glycero-3-phosphoserine; 1-(9Z,12Z,15Z-octadecatrienoyl)-2-docosanoyl-glycero-3-phosphoserine; 1-(9Z,12Z,15Z-octadecatrienoyl)-2-(11Z-docosenoyl)-glycero-3-phosphoserine; 1-(9Z,12Z,15Z-octadecatrienoyl)-2-(13Z,16Z-docosadienoyl)-glycero-3-phosphoserine; 1-(9Z,12Z,15Z-octadecatrienoyl)-2-(7Z,10Z,13Z,16Z-docosatetraenoyl)-glycero-3-phosphoserine; 1-(9Z,12Z,15Z-octadecatrienoyl)-2-(4Z,7Z,10Z,13Z,16Z,19Z-docosahexaenoyl)-glycero-3-phosphoserine; 1-(6Z,9Z,12Z,15Z-octadecatetraenoyl)-glycero-3-phosphoserine; 1-(6Z,9Z,12Z,15Z-octadecatetraenoyl)-2-dodecanoyl-glycero-3-phosphoserine; 1-(6Z,9Z,12Z,15Z-octadecatetraenoyl)-2-tridecanoyl-glycero-3-phosphoserine; 1-(6Z,9Z,12Z,15Z-octadecatetraenoyl)-2-tetradecanoyl-glycero-3-phosphoserine; 1-(6Z,9Z,12Z,15Z-octadecatetraenoyl)-2-(9Z-tetradecenoyl)-glycero-3-phosphoserine; 1-(6Z,9Z,12Z,15Z-octadecatetraenoyl)-2-pentadecanoyl-glycero-3-phosphoserine; 1-(6Z,9Z,12Z,15Z-octadecatetraenoyl)-2-(9Z-pentadecenoyl)-glycero-3-phosphoserine; 1-(6Z,9Z,12Z,15Z-octadecatetraenoyl)-2-hexadecanoyl-glycero-3-phosphoserine; 1-(6Z,9Z,12Z,15Z-octadecatetraenoyl)-2-(9Z-hexadecenoyl)-glycero-3-phosphoserine; 1-(6Z,9Z,12Z,15Z-octadecatetraenoyl)-2-heptadecanoyl-glycero-3-phosphoserine; 1-(6Z,9Z,12Z,15Z-octadecatetraenoyl)-2-(9Z-heptadecenoyl)-glycero-3-phosphoserine; 1-(6Z,9Z,12Z,15Z-octadecatetraenoyl)-2-(9Z,12Z-heptadecadienoyl)-glycero-3-phosphoserine; 1-(6Z,9Z,12Z,15Z-octadecatetraenoyl)-2-octadecanoyl-glycero-3-phosphoserine; 1-(6Z,9Z,12Z,15Z-octadecatetraenoyl)-2-(9Z-octadecenoyl)-glycero-3-phosphoserine; 1-(6Z,9Z,12Z,15Z-octadecatetraenoyl)-2-(9Z,12Z-octadecadienoyl)-glycero-3-phosphoserine; 1-(6Z,9Z,12Z,15Z-octadecatetraenoyl)-2-(6Z,9Z,12Z-octadecatrienoyl)-glycero-3-phosphoserine; 1-(6Z,9Z,12Z,15Z-octadecatetraenoyl)-2-(9Z,12Z,15Z-octadecatrienoyl)-glycero-3-phosphoserine; 1,2-di-(6Z,9Z,12Z,15Z-octadecatetraenoyl)-sn-glycero-3-phosphoserine; 1-(6Z,9Z,12Z,15Z-octadecatetraenoyl)-2-nonadecanoyl-glycero-3-phosphoserine; 1-(6Z,9Z,12Z,15Z-octadecatetraenoyl)-2-(9Z-nonadecenoyl)-glycero-3-phosphoserine; 1-(6Z,9Z,12Z,15Z-octadecatetraenoyl)-2-eicosanoyl-glycero-3-phosphoserine; 1-(6Z,9Z,12Z,15Z-octadecatetraenoyl)-2-(11Z-eicosenoyl)-glycero-3-phosphoserine; 1-(6Z,9Z,12Z,15Z-octadecatetraenoyl)-2-(11Z,14Z-eicosadienoyl)-glycero-3-phosphoserine; 1-(6Z,9Z,12Z,15Z-octadecatetraenoyl)-2-(8Z,11Z,14Z-eicosatrienoyl)-glycero-3-phosphoserine; 1-(6Z,9Z,12Z,15Z-octadecatetraenoyl)-2-(5Z,8Z,11Z,14Z-eicosatetraenoyl)-glycero-3-phosphoserine; 1-(6Z,9Z,12Z,15Z-octadecatetraenoyl)-2-(5Z,8Z,11Z,14Z,17Z-eicosapentaenoyl)-glycero-3-phosphoserine; 1-(6Z,9Z,12Z,15Z-octadecatetraenoyl)-2-heneicosanoyl-glycero-3-phosphoserine; 1-(6Z,9Z,12Z,15Z-octadecatetraenoyl)-2-docosanoyl-glycero-3-phosphoserine; 1-(6Z,9Z,12Z,15Z-octadecatetraenoyl)-2-(11Z-docosenoyl)-glycero-3-phosphoserine; 1-(6Z,9Z,12Z,15Z-octadecatetraenoyl)-2-(13Z,16Z-docosadienoyl)-glycero-3-phosphoserine; 1-(6Z,9Z,12Z,15Z-octadecatetraenoyl)-2-(7Z,10Z,13Z,16Z-docosatetraenoyl)-glycero-3-phosphoserine; 1-(6Z,9Z,12Z,15Z-octadecatetraenoyl)-2-(4Z,7Z,10Z,13Z,16Z,19Z-docosahexaenoyl)-glycero-3-phosphoserine; 1-nonadecanoyl-2-(9Z-tetradecenoyl)-glycero-3-phosphoserine; 1-nonadecanoyl-2-(9Z-pentadecenoyl)-glycero-3-phosphoserine; 1-nonadecanoyl-2-(9Z-hexadecenoyl)-glycero-3-phosphoserine; 1-nonadecanoyl-2-heptadecanoyl-glycero-3-phosphoserine; 1-nonadecanoyl-2-(9Z-heptadecenoyl)-glycero-3-phosphoserine; 1-nonadecanoyl-2-(9Z,12Z-heptadecadienoyl)-glycero-3-phosphoserine; 1-nonadecanoyl-2-(9Z-octadecenoyl)-glycero-3-phosphoserine; 1-nonadecanoyl-2-(9Z,12Z-octadecadienoyl)-glycero-3-phosphoserine; 1-nonadecanoyl-2-(6Z,9Z,12Z-octadecatrienoyl)-glycero-3-phosphoserine; 1-nonadecanoyl-2-(9Z,12Z,15Z-octadecatrienoyl)-glycero-3-phosphoserine; 1-nonadecanoyl-2-(6Z,9Z,12Z,15Z-octadecatetraenoyl)-glycero-3-phosphoserine; 1-nonadecanoyl-2-(9Z-nonadecenoyl)-glycero-3-phosphoserine; 1-nonadecanoyl-2-eicosanoyl-glycero-3-phosphoserine; 1-nonadecanoyl-2-(11Z-eicosenoyl)-glycero-3-phosphoserine; 1-nonadecanoyl-2-(11Z,14Z-eicosadienoyl)-glycero-3-phosphoserine; 1-nonadecanoyl-2-(8Z,11Z,14Z-eicosatrienoyl)-glycero-3-phosphoserine; 1-nonadecanoyl-2-(5Z,8Z,11Z,14Z-eicosatetraenoyl)-glycero-3-phosphoserine; 1-nonadecanoyl-2-(5Z,8Z,11Z,14Z,17Z-eicosapentaenoyl)-glycero-3-phosphoserine; 1-nonadecanoyl-2-heneicosanoyl-glycero-3-phosphoserine; 1-nonadecanoyl-2-docosanoyl-glycero-3-phosphoserine; 1-nonadecanoyl-2-(11Z-docosenoyl)-glycero-3-phosphoserine; 1-nonadecanoyl-2-(13Z,16Z-docosadienoyl)-glycero-3-phosphoserine; 1-nonadecanoyl-2-(7Z,10Z,13Z,16Z-docosatetraenoyl)-glycero-3-phosphoserine; 1-nonadecanoyl-2-(4Z,7Z,10Z,13Z,16Z,19Z-docosahexaenoyl)-glycero-3-phosphoserine; 1-(9Z-nonadecenoyl)-glycero-3-phosphoserine; 1-(9Z-nonadecenoyl)-2-dodecanoyl-glycero-3-phosphoserine; 1-(9Z-nonadecenoyl)-2-tridecanoyl-glycero-3-phosphoserine; 1-(9Z-nonadecenoyl)-2-tetradecanoyl-glycero-3-phosphoserine; 1-(9Z-nonadecenoyl)-2-(9Z-tetradecenoyl)-glycero-3-phosphoserine; 1-(9Z-nonadecenoyl)-2-pentadecanoyl-glycero-3-phosphoserine; 1-(9Z-nonadecenoyl)-2-(9Z-pentadecenoyl)-glycero-3-phosphoserine; 1-(9Z-nonadecenoyl)-2-hexadecanoyl-glycero-3-phosphoserine; 1-(9Z-nonadecenoyl)-2-(9Z-hexadecenoyl)-glycero-3-phosphoserine; 1-(9Z-nonadecenoyl)-2-heptadecanoyl-glycero-3-phosphoserine; 1-(9Z-nonadecenoyl)-2-(9Z-heptadecenoyl)-glycero-3-phosphoserine; 1-(9Z-nonadecenoyl)-2-(9Z,12Z-heptadecadienoyl)-glycero-3-phosphoserine; 1-(9Z-nonadecenoyl)-2-octadecanoyl-glycero-3-phosphoserine; 1-(9Z-nonadecenoyl)-2-(9Z-octadecenoyl)-glycero-3-phosphoserine; 1-(9Z-nonadecenoyl)-2-(9Z,12Z-octadecadienoyl)-glycero-3-phosphoserine; 1-(9Z-nonadecenoyl)-2-(6Z,9Z,12Z-octadecatrienoyl)-glycero-3-phosphoserine; 1-(9Z-nonadecenoyl)-2-(9Z,12Z,15Z-octadecatrienoyl)-glycero-3-phosphoserine; 1-(9Z-nonadecenoyl)-2-(6Z,9Z,12Z,15Z-octadecatetraenoyl)-glycero-3-phosphoserine; 1-(9Z-nonadecenoyl)-2-nonadecanoyl-glycero-3-phosphoserine; 1,2-di-(9Z-nonadecenoyl)-sn-glycero-3-phosphoserine; 1-(9Z-nonadecenoyl)-2-eicosanoyl-glycero-3-phosphoserine; 1-(9Z-nonadecenoyl)-2-(11Z-eicosenoyl)-glycero-3-phosphoserine; 1-(9Z-nonadecenoyl)-2-(11Z,14Z-eicosadienoyl)-glycero-3-phosphoserine; 1-(9Z-nonadecenoyl)-2-(8Z,11Z,14Z-eicosatrienoyl)-glycero-3-phosphoserine; 1-(9Z-nonadecenoyl)-2-(5Z,8Z,11Z,14Z-eicosatetraenoyl)-glycero-3-phosphoserine; 1-(9Z-nonadecenoyl)-2-(5Z,8Z,11Z,14Z,17Z-eicosapentaenoyl)-glycero-3-phosphoserine; 1-(9Z-nonadecenoyl)-2-heneicosanoyl-glycero-3-phosphoserine; 1-(9Z-nonadecenoyl)-2-docosanoyl-glycero-3-phosphoserine; 1-(9Z-nonadecenoyl)-2-(11Z-docosenoyl)-glycero-3-phosphoserine; 1-(9Z-nonadecenoyl)-2-(13Z,16Z-docosadienoyl)-glycero-3-phosphoserine; 1-(9Z-nonadecenoyl)-2-(7Z,10Z,13Z,16Z-docosatetraenoyl)-glycero-3-phosphoserine; 1-(9Z-nonadecenoyl)-2-(4Z,7Z,10Z,13Z,16Z,19Z-docosahexaenoyl)-glycero-3-phosphoserine; 1-eicosanoyl-2-(9Z-tetradecenoyl)-glycero-3-phosphoserine; 1-eicosanoyl-2-pentadecanoyl-glycero-3-phosphoserine; 1-eicosanoyl-2-(9Z-pentadecenoyl)-glycero-3-phosphoserine; 1-eicosanoyl-2-(9Z-hexadecenoyl)-glycero-3-phosphoserine; 1-eicosanoyl-2-heptadecanoyl-glycero-3-phosphoserine; 1-eicosanoyl-2-(9Z-heptadecenoyl)-glycero-3-phosphoserine; 1-eicosanoyl-2-(9Z,12Z-heptadecadienoyl)-glycero-3-phosphoserine; 1-eicosanoyl-2-(6Z,9Z,12Z-octadecatrienoyl)-glycero-3-phosphoserine; 1-eicosanoyl-2-(9Z,12Z,15Z-octadecatrienoyl)-glycero-3-phosphoserine; 1-eicosanoyl-2-nonadecanoyl-glycero-3-phosphoserine; 1-eicosanoyl-2-(9Z-nonadecenoyl)-glycero-3-phosphoserine; 1-eicosanoyl-2-(8Z,11Z,14Z-eicosatrienoyl)-glycero-3-phosphoserine; 1-eicosanoyl-2-(5Z,8Z,11Z,14Z,17Z-eicosapentaenoyl)-glycero-3-phosphoserine; 1-eicosanoyl-2-heneicosanoyl-glycero-3-phosphoserine; 1-eicosanoyl-2-(11Z-docosenoyl)-glycero-3-phosphoserine; 1-eicosanoyl-2-(13Z,16Z-docosadienoyl)-glycero-3-phosphoserine; 1-eicosanoyl-2-(7Z,10Z,13Z,16Z-docosatetraenoyl)-glycero-3-phosphoserine; 1-(11Z-eicosenoyl)-glycero-3-phosphoserine; 1-(11Z-eicosenoyl)-2-dodecanoyl-glycero-3-phosphoserine; 1-(11Z-eicosenoyl)-2-tridecanoyl-glycero-3-phosphoserine; 1-(11Z-eicosenoyl)-2-tetradecanoyl-glycero-3-phosphoserine; 1-(11Z-eicosenoyl)-2-(9Z-tetradecenoyl)-glycero-3-phosphoserine; 1-(11Z-eicosenoyl)-2-pentadecanoyl-glycero-3-phosphoserine; 1-(11Z-eicosenoyl)-2-(9Z-pentadecenoyl)-glycero-3-phosphoserine; 1-(11Z-eicosenoyl)-2-hexadecanoyl-glycero-3-phosphoserine; 1-(11Z-eicosenoyl)-2-(9Z-hexadecenoyl)-glycero-3-phosphoserine; 1-(11Z-eicosenoyl)-2-heptadecanoyl-glycero-3-phosphoserine; 1-(11Z-eicosenoyl)-2-(9Z-heptadecenoyl)-glycero-3-phosphoserine; 1-(11Z-eicosenoyl)-2-(9Z,12Z-heptadecadienoyl)-glycero-3-phosphoserine; 1-(11Z-eicosenoyl)-2-octadecanoyl-glycero-3-phosphoserine; 1-(11Z-eicosenoyl)-2-(9Z-octadecenoyl)-glycero-3-phosphoserine; 1-(11Z-eicosenoyl)-2-(9Z,12Z-octadecadienoyl)-glycero-3-phosphoserine; 1-(11Z-eicosenoyl)-2-(6Z,9Z,12Z-octadecatrienoyl)-glycero-3-phosphoserine; 1-(11Z-eicosenoyl)-2-(9Z,12Z,15Z-octadecatrienoyl)-glycero-3-phosphoserine; 1-(11Z-eicosenoyl)-2-(6Z,9Z,12Z,15Z-octadecatetraenoyl)-glycero-3-phosphoserine; 1-(11Z-eicosenoyl)-2-nonadecanoyl-glycero-3-phosphoserine; 1-(11Z-eicosenoyl)-2-(9Z-nonadecenoyl)-glycero-3-phosphoserine; 1-(11Z-eicosenoyl)-2-eicosanoyl-glycero-3-phosphoserine; 1-(11Z-eicosenoyl)-2-(11Z,14Z-eicosadienoyl)-glycero-3-phosphoserine; 1-(11Z-eicosenoyl)-2-(8Z,11Z,14Z-eicosatrienoyl)-glycero-3-phosphoserine; 1-(11Z-eicosenoyl)-2-(5Z,8Z,11Z,14Z-eicosatetraenoyl)-glycero-3-phosphoserine; 1-(11Z-eicosenoyl)-2-(5Z,8Z,11Z,14Z,17Z-eicosapentaenoyl)-glycero-3-phosphoserine; 1-(11Z-eicosenoyl)-2-heneicosanoyl-glycero-3-phosphoserine; 1-(11Z-eicosenoyl)-2-docosanoyl-glycero-3-phosphoserine; 1-(11Z-eicosenoyl)-2-(11Z-docosenoyl)-glycero-3-phosphoserine; 1-(11Z-eicosenoyl)-2-(13Z,16Z-docosadienoyl)-glycero-3-phosphoserine; 1-(11Z-eicosenoyl)-2-(7Z,10Z,13Z,16Z-docosatetraenoyl)-glycero-3-phosphoserine; 1-(11Z-eicosenoyl)-2-(4Z,7Z,10Z,13Z,16Z,19Z-docosahexaenoyl)-glycero-3-phosphoserine; 1-(11Z,14Z-eicosadienoyl)-glycero-3-phosphoserine; 1-(11Z,14Z-eicosadienoyl)-2-dodecanoyl-glycero-3-phosphoserine; 1-(11Z,14Z-eicosadienoyl)-2-tridecanoyl-glycero-3-phosphoserine; 1-(11Z,14Z-eicosadienoyl)-2-tetradecanoyl-glycero-3-phosphoserine; 1-(11Z,14Z-eicosadienoyl)-2-(9Z-tetradecenoyl)-glycero-3-phosphoserine; 1-(11Z,14Z-eicosadienoyl)-2-pentadecanoyl-glycero-3-phosphoserine; 1-(11Z,14Z-eicosadienoyl)-2-(9Z-pentadecenoyl)-glycero-3-phosphoserine; 1-(11Z,14Z-eicosadienoyl)-2-hexadecanoyl-glycero-3-phosphoserine; 1-(11Z,14Z-eicosadienoyl)-2-(9Z-hexadecenoyl)-glycero-3-phosphoserine; 1-(11Z,14Z-eicosadienoyl)-2-heptadecanoyl-glycero-3-phosphoserine; 1-(11Z,14Z-eicosadienoyl)-2-(9Z-heptadecenoyl)-glycero-3-phosphoserine; 1-(11Z,14Z-eicosadienoyl)-2-(9Z,12Z-heptadecadienoyl)-glycero-3-phosphoserine; 1-(11Z,14Z-eicosadienoyl)-2-octadecanoyl-glycero-3-phosphoserine; 1-(11Z,14Z-eicosadienoyl)-2-(9Z-octadecenoyl)-glycero-3-phosphoserine; 1-(11Z,14Z-eicosadienoyl)-2-(9Z,12Z-octadecadienoyl)-glycero-3-phosphoserine; 1-(11Z,14Z-eicosadienoyl)-2-(6Z,9Z,12Z-octadecatrienoyl)-glycero-3-phosphoserine; 1-(11Z,14Z-eicosadienoyl)-2-(9Z,12Z,15Z-octadecatrienoyl)-glycero-3-phosphoserine; 1-(11Z,14Z-eicosadienoyl)-2-(6Z,9Z,12Z,15Z-octadecatetraenoyl)-glycero-3-phosphoserine; 1-(11Z,14Z-eicosadienoyl)-2-nonadecanoyl-glycero-3-phosphoserine; 1-(11Z,14Z-eicosadienoyl)-2-(9Z-nonadecenoyl)-glycero-3-phosphoserine; 1-(11Z,14Z-eicosadienoyl)-2-eicosanoyl-glycero-3-phosphoserine; 1-(11Z,14Z-eicosadienoyl)-2-(11Z-eicosenoyl)-glycero-3-phosphoserine; 1,2-di-(11Z,14Z-eicosadienoyl)-sn-glycero-3-phosphoserine; 1-(11Z,14Z-eicosadienoyl)-2-(8Z,11Z,14Z-eicosatrienoyl)-glycero-3-phosphoserine; 1-(11Z,14Z-eicosadienoyl)-2-(5Z,8Z,11Z,14Z-eicosatetraenoyl)-glycero-3-phosphoserine; 1-(11Z,14Z-eicosadienoyl)-2-(5Z,8Z,11Z,14Z,17Z-eicosapentaenoyl)-glycero-3-phosphoserine; 1-(11Z,14Z-eicosadienoyl)-2-heneicosanoyl-glycero-3-phosphoserine; 1-(11Z,14Z-eicosadienoyl)-2-docosanoyl-glycero-3-phosphoserine; 1-(11Z,14Z-eicosadienoyl)-2-(11Z-docosenoyl)-glycero-3-phosphoserine; 1-(11Z,14Z-eicosadienoyl)-2-(13Z,16Z-docosadienoyl)-glycero-3-phosphoserine; 1-(11Z,14Z-eicosadienoyl)-2-(7Z,10Z,13Z,16Z-docosatetraenoyl)-glycero-3-phosphoserine; 1-(11Z,14Z-eicosadienoyl)-2-(4Z,7Z,10Z,13Z,16Z,19Z-docosahexaenoyl)-glycero-3-phosphoserine; 1-(8Z,11Z,14Z-eicosatrienoyl)-glycero-3-phosphoserine; 1-(8Z,11Z,14Z-eicosatrienoyl)-2-dodecanoyl-glycero-3-phosphoserine; 1-(8Z,11Z,14Z-eicosatrienoyl)-2-tridecanoyl-glycero-3-phosphoserine; 1-(8Z,11Z,14Z-eicosatrienoyl)-2-tetradecanoyl-glycero-3-phosphoserine; 1-(8Z,11Z,14Z-eicosatrienoyl)-2-(9Z-tetradecenoyl)-glycero-3-phosphoserine; 1-(8Z,11Z,14Z-eicosatrienoyl)-2-pentadecanoyl-glycero-3-phosphoserine; 1-(8Z,11Z,14Z-eicosatrienoyl)-2-(9Z-pentadecenoyl)-glycero-3-phosphoserine; 1-(8Z,11Z,14Z-eicosatrienoyl)-2-hexadecanoyl-glycero-3-phosphoserine; 1-(8Z,11Z,14Z-eicosatrienoyl)-2-(9Z-hexadecenoyl)-glycero-3-phosphoserine; 1-(8Z,11Z,14Z-eicosatrienoyl)-2-heptadecanoyl-glycero-3-phosphoserine; 1-(8Z,11Z,14Z-eicosatrienoyl)-2-(9Z-heptadecenoyl)-glycero-3-phosphoserine; 1-(8Z,11Z,14Z-eicosatrienoyl)-2-(9Z,12Z-heptadecadienoyl)-glycero-3-phosphoserine; 1-(8Z,11Z,14Z-eicosatrienoyl)-2-octadecanoyl-glycero-3-phosphoserine; 1-(8Z,11Z,14Z-eicosatrienoyl)-2-(9Z-octadecenoyl)-glycero-3-phosphoserine; 1-(8Z,11Z,14Z-eicosatrienoyl)-2-(9Z,12Z-octadecadienoyl)-glycero-3-phosphoserine; 1-(8Z,11Z,14Z-eicosatrienoyl)-2-(6Z,9Z,12Z-octadecatrienoyl)-glycero-3-phosphoserine; 1-(8Z,11Z,14Z-eicosatrienoyl)-2-(9Z,12Z,15Z-octadecatrienoyl)-glycero-3-phosphoserine; 1-(8Z,11Z,14Z-eicosatrienoyl)-2-(6Z,9Z,12Z,15Z-octadecatetraenoyl)-glycero-3-phosphoserine; 1-(8Z,11Z,14Z-eicosatrienoyl)-2-nonadecanoyl-glycero-3-phosphoserine; 1-(8Z,11Z,14Z-eicosatrienoyl)-2-(9Z-nonadecenoyl)-glycero-3-phosphoserine; 1-(8Z,11Z,14Z-eicosatrienoyl)-2-eicosanoyl-glycero-3-phosphoserine; 1-(8Z,11Z,14Z-eicosatrienoyl)-2-(11Z-eicosenoyl)-glycero-3-phosphoserine; 1-(8Z,11Z,14Z-eicosatrienoyl)-2-(11Z,14Z-eicosadienoyl)-glycero-3-phosphoserine; 1,2-di-(8Z,11Z,14Z-eicosatrienoyl)-sn-glycero-3-phosphoserine; 1-(8Z,11Z,14Z-eicosatrienoyl)-2-(5Z,8Z,11Z,14Z-eicosatetraenoyl)-glycero-3-phosphoserine; 1-(8Z,11Z,14Z-eicosatrienoyl)-2-(5Z,8Z,11Z,14Z,17Z-eicosapentaenoyl)-glycero-3-phosphoserine; 1-(8Z,11Z,14Z-eicosatrienoyl)-2-heneicosanoyl-glycero-3-phosphoserine; 1-(8Z,11Z,14Z-eicosatrienoyl)-2-docosanoyl-glycero-3-phosphoserine; 1-(8Z,11Z,14Z-eicosatrienoyl)-2-(11Z-docosenoyl)-glycero-3-phosphoserine; 1-(8Z,11Z,14Z-eicosatrienoyl)-2-(13Z,16Z-docosadienoyl)-glycero-3-phosphoserine; 1-(8Z,11Z,14Z-eicosatrienoyl)-2-(7Z,10Z,13Z,16Z-docosatetraenoyl)-glycero-3-phosphoserine; 1-(8Z,11Z,14Z-eicosatrienoyl)-2-(4Z,7Z,10Z,13Z,16Z,19Z-docosahexaenoyl)-glycero-3-phosphoserine; 1-(5Z,8Z,11Z,14Z-eicosatetraenoyl)-2-dodecanoyl-glycero-3-phosphoserine; 1-(5Z,8Z,11Z,14Z-eicosatetraenoyl)-2-tridecanoyl-glycero-3-phosphoserine; 1-(5Z,8Z,11Z,14Z-eicosatetraenoyl)-2-tetradecanoyl-glycero-3-phosphoserine; 1-(5Z,8Z,11Z,14Z-eicosatetraenoyl)-2-(9Z-tetradecenoyl)-glycero-3-phosphoserine; 1-(5Z,8Z,11Z,14Z-eicosatetraenoyl)-2-pentadecanoyl-glycero-3-phosphoserine; 1-(5Z,8Z,11Z,14Z-eicosatetraenoyl)-2-(9Z-pentadecenoyl)-glycero-3-phosphoserine; 1-(5Z,8Z,11Z,14Z-eicosatetraenoyl)-2-(9Z-hexadecenoyl)-glycero-3-phosphoserine; 1-(5Z,8Z,11Z,14Z-eicosatetraenoyl)-2-heptadecanoyl-glycero-3-phosphoserine; 1-(5Z,8Z,11Z,14Z-eicosatetraenoyl)-2-(9Z-heptadecenoyl)-glycero-3-phosphoserine; 1-(5Z,8Z,11Z,14Z-eicosatetraenoyl)-2-(9Z,12Z-heptadecadienoyl)-glycero-3-phosphoserine; 1-(5Z,8Z,11Z,14Z-eicosatetraenoyl)-2-octadecanoyl-glycero-3-phosphoserine; 1-(5Z,8Z,11Z,14Z-eicosatetraenoyl)-2-(9Z-octadecenoyl)-glycero-3-phosphoserine; 1-(5Z,8Z,11Z,14Z-eicosatetraenoyl)-2-(9Z,12Z-octadecadienoyl)-glycero-3-phosphoserine; 1-(5Z,8Z,11Z,14Z-eicosatetraenoyl)-2-(6Z,9Z,12Z-octadecatrienoyl)-glycero-3-phosphoserine; 1-(5Z,8Z,11Z,14Z-eicosatetraenoyl)-2-(9Z,12Z,15Z-octadecatrienoyl)-glycero-3-phosphoserine; 1-(5Z,8Z,11Z,14Z-eicosatetraenoyl)-2-(6Z,9Z,12Z,15Z-octadecatetraenoyl)-glycero-3-phosphoserine; 1-(5Z,8Z,11Z,14Z-eicosatetraenoyl)-2-nonadecanoyl-glycero-3-phosphoserine; 1-(5Z,8Z,11Z,14Z-eicosatetraenoyl)-2-(9Z-nonadecenoyl)-glycero-3-phosphoserine; 1-(5Z,8Z,11Z,14Z-eicosatetraenoyl)-2-eicosanoyl-glycero-3-phosphoserine; 1-(5Z,8Z,11Z,14Z-eicosatetraenoyl)-2-(11Z-eicosenoyl)-glycero-3-phosphoserine; 1-(5Z,8Z,11Z,14Z-eicosatetraenoyl)-2-(11Z,14Z-eicosadienoyl)-glycero-3-phosphoserine; 1-(5Z,8Z,11Z,14Z-eicosatetraenoyl)-2-(8Z,11Z,14Z-eicosatrienoyl)-glycero-3-phosphoserine; 1-(5Z,8Z,11Z,14Z-eicosatetraenoyl)-2-(5Z,8Z,11Z,14Z,17Z-eicosapentaenoyl)-glycero-3-phosphoserine; 1-(5Z,8Z,11Z,14Z-eicosatetraenoyl)-2-heneicosanoyl-glycero-3-phosphoserine; 1-(5Z,8Z,11Z,14Z-eicosatetraenoyl)-2-docosanoyl-glycero-3-phosphoserine; 1-(5Z,8Z,11Z,14Z-eicosatetraenoyl)-2-(11Z-docosenoyl)-glycero-3-phosphoserine; 1-(5Z,8Z,11Z,14Z-eicosatetraenoyl)-2-(13Z,16Z-docosadienoyl)-glycero-3-phosphoserine; 1-(5Z,8Z,11Z,14Z-eicosatetraenoyl)-2-(7Z,10Z,13Z,16Z-docosatetraenoyl)-glycero-3-phosphoserine; 1-(5Z,8Z,11Z,14Z,17Z-eicosapentaenoyl)-2-dodecanoyl-glycero-3-phosphoserine; 1-(5Z,8Z,11Z,14Z,17Z-eicosapentaenoyl)-2-tridecanoyl-glycero-3-phosphoserine; 1-(5Z,8Z,11Z,14Z,17Z-eicosapentaenoyl)-2-tetradecanoyl-glycero-3-phosphoserine; 1-(5Z,8Z,11Z,14Z,17Z-eicosapentaenoyl)-2-(9Z-tetradecenoyl)-glycero-3-phosphoserine; 1-(5Z,8Z,11Z,14Z,17Z-eicosapentaenoyl)-2-pentadecanoyl-glycero-3-phosphoserine; 1-(5Z,8Z,11Z,14Z,17Z-eicosapentaenoyl)-2-(9Z-pentadecenoyl)-glycero-3-phosphoserine; 1-(5Z,8Z,11Z,14Z,17Z-eicosapentaenoyl)-2-hexadecanoyl-glycero-3-phosphoserine; 1-(5Z,8Z,11Z,14Z,17Z-eicosapentaenoyl)-2-(9Z-hexadecenoyl)-glycero-3-phosphoserine; 1-(5Z,8Z,11Z,14Z,17Z-eicosapentaenoyl)-2-heptadecanoyl-glycero-3-phosphoserine; 1-(5Z,8Z,11Z,14Z,17Z-eicosapentaenoyl)-2-(9Z-heptadecenoyl)-glycero-3-phosphoserine; 1-(5Z,8Z,11Z,14Z,17Z-eicosapentaenoyl)-2-(9Z,12Z-heptadecadienoyl)-glycero-3-phosphoserine; 1-(5Z,8Z,11Z,14Z,17Z-eicosapentaenoyl)-2-octadecanoyl-glycero-3-phosphoserine; 1-(5Z,8Z,11Z,14Z,17Z-eicosapentaenoyl)-2-(9Z,12Z-octadecadienoyl)-glycero-3-phosphoserine; 1-(5Z,8Z,11Z,14Z,17Z-eicosapentaenoyl)-2-(6Z,9Z,12Z-octadecatrienoyl)-glycero-3-phosphoserine; 1-(5Z,8Z,11Z,14Z,17Z-eicosapentaenoyl)-2-(9Z,12Z,15Z-octadecatrienoyl)-glycero-3-phosphoserine; 1-(5Z,8Z,11Z,14Z,17Z-eicosapentaenoyl)-2-(6Z,9Z,12Z,15Z-octadecatetraenoyl)-glycero-3-phosphoserine; 1-(5Z,8Z,11Z,14Z,17Z-eicosapentaenoyl)-2-nonadecanoyl-glycero-3-phosphoserine; 1-(5Z,8Z,11Z,14Z,17Z-eicosapentaenoyl)-2-(9Z-nonadecenoyl)-glycero-3-phosphoserine; 1-(5Z,8Z,11Z,14Z,17Z-eicosapentaenoyl)-2-eicosanoyl-glycero-3-phosphoserine; 1-(5Z,8Z,11Z,14Z,17Z-eicosapentaenoyl)-2-(11Z-eicosenoyl)-glycero-3-phosphoserine; 1-(5Z,8Z,11Z,14Z,17Z-eicosapentaenoyl)-2-(11Z,14Z-eicosadienoyl)-glycero-3-phosphoserine; 1-(5Z,8Z,11Z,14Z,17Z-eicosapentaenoyl)-2-(8Z,11Z,14Z-eicosatrienoyl)-glycero-3-phosphoserine; 1-(5Z,8Z,11Z,14Z,17Z-eicosapentaenoyl)-2-(5Z,8Z,11Z,14Z-eicosatetraenoyl)-glycero-3-phosphoserine; 1,2-di-(5Z,8Z,11Z,14Z,17Z-eicosapentaenoyl)-sn-glycero-3-phosphoserine; 1-(5Z,8Z,11Z,14Z,17Z-eicosapentaenoyl)-2-heneicosanoyl-glycero-3-phosphoserine; 1-(5Z,8Z,11Z,14Z,17Z-eicosapentaenoyl)-2-docosanoyl-glycero-3-phosphoserine; 1-(5Z,8Z,11Z,14Z,17Z-eicosapentaenoyl)-2-(11Z-docosenoyl)-glycero-3-phosphoserine; 1-(5Z,8Z,11Z,14Z,17Z-eicosapentaenoyl)-2-(13Z,16Z-docosadienoyl)-glycero-3-phosphoserine; 1-(5Z,8Z,11Z,14Z,17Z-eicosapentaenoyl)-2-(7Z,10Z,13Z,16Z-docosatetraenoyl)-glycero-3-phosphoserine; 1-heneicosanoyl-2-(9Z-tetradecenoyl)-glycero-3-phosphoserine; 1-heneicosanoyl-2-pentadecanoyl-glycero-3-phosphoserine; 1-heneicosanoyl-2-(9Z-pentadecenoyl)-glycero-3-phosphoserine; 1-heneicosanoyl-2-hexadecanoyl-glycero-3-phosphoserine; 1-heneicosanoyl-2-(9Z-hexadecenoyl)-glycero-3-phosphoserine; 1-heneicosanoyl-2-heptadecanoyl-glycero-3-phosphoserine; 1-heneicosanoyl-2-(9Z-heptadecenoyl)-glycero-3-phosphoserine; 1-heneicosanoyl-2-(9Z,12Z-heptadecadienoyl)-glycero-3-phosphoserine; 1-heneicosanoyl-2-(9Z-octadecenoyl)-glycero-3-phosphoserine; 1-heneicosanoyl-2-(9Z,12Z-octadecadienoyl)-glycero-3-phosphoserine; 1-heneicosanoyl-2-(6Z,9Z,12Z-octadecatrienoyl)-glycero-3-phosphoserine; 1-heneicosanoyl-2-(9Z,12Z,15Z-octadecatrienoyl)-glycero-3-phosphoserine; 1-heneicosanoyl-2-(6Z,9Z,12Z,15Z-octadecatetraenoyl)-glycero-3-phosphoserine; 1-heneicosanoyl-2-nonadecanoyl-glycero-3-phosphoserine; 1-heneicosanoyl-2-(9Z-nonadecenoyl)-glycero-3-phosphoserine; 1-heneicosanoyl-2-eicosanoyl-glycero-3-phosphoserine; 1-heneicosanoyl-2-(11Z-eicosenoyl)-glycero-3-phosphoserine; 1-heneicosanoyl-2-(11Z,14Z-eicosadienoyl)-glycero-3-phosphoserine; 1-heneicosanoyl-2-(8Z,11Z,14Z-eicosatrienoyl)-glycero-3-phosphoserine; 1-heneicosanoyl-2-(5Z,8Z,11Z,14Z-eicosatetraenoyl)-glycero-3-phosphoserine; 1-heneicosanoyl-2-(5Z,8Z,11Z,14Z,17Z-eicosapentaenoyl)-glycero-3-phosphoserine; 1-heneicosanoyl-2-docosanoyl-glycero-3-phosphoserine; 1-heneicosanoyl-2-(11Z-docosenoyl)-glycero-3-phosphoserine; 1-heneicosanoyl-2-(13Z,16Z-docosadienoyl)-glycero-3-phosphoserine; 1-heneicosanoyl-2-(7Z,10Z,13Z,16Z-docosatetraenoyl)-glycero-3-phosphoserine; 1-docosanoyl-2-(9Z-tetradecenoyl)-glycero-3-phosphoserine; 1-docosanoyl-2-pentadecanoyl-glycero-3-phosphoserine; 1-docosanoyl-2-(9Z-pentadecenoyl)-glycero-3-phosphoserine; 1-docosanoyl-2-hexadecanoyl-glycero-3-phosphoserine; 1-docosanoyl-2-(9Z-hexadecenoyl)-glycero-3-phosphoserine; 1-docosanoyl-2-heptadecanoyl-glycero-3-phosphoserine; 1-docosanoyl-2-(9Z-heptadecenoyl)-glycero-3-phosphoserine; 1-docosanoyl-2-(9Z,12Z-heptadecadienoyl)-glycero-3-phosphoserine; 1-docosanoyl-2-(9Z,12Z-octadecadienoyl)-glycero-3-phosphoserine; 1-docosanoyl-2-(6Z,9Z,12Z-octadecatrienoyl)-glycero-3-phosphoserine; 1-docosanoyl-2-(9Z,12Z,15Z-octadecatrienoyl)-glycero-3-phosphoserine; 1-docosanoyl-2-(6Z,9Z,12Z,15Z-octadecatetraenoyl)-glycero-3-phosphoserine; 1-docosanoyl-2-nonadecanoyl-glycero-3-phosphoserine; 1-docosanoyl-2-(9Z-nonadecenoyl)-glycero-3-phosphoserine; 1-docosanoyl-2-eicosanoyl-glycero-3-phosphoserine; 1-docosanoyl-2-(11Z,14Z-eicosadienoyl)-glycero-3-phosphoserine; 1-docosanoyl-2-(8Z,11Z,14Z-eicosatrienoyl)-glycero-3-phosphoserine; 1-docosanoyl-2-(5Z,8Z,11Z,14Z-eicosatetraenoyl)-glycero-3-phosphoserine; 1-docosanoyl-2-(5Z,8Z,11Z,14Z,17Z-eicosapentaenoyl)-glycero-3-phosphoserine; 1-docosanoyl-2-heneicosanoyl-glycero-3-phosphoserine; 1-docosanoyl-2-(11Z-docosenoyl)-glycero-3-phosphoserine; 1-docosanoyl-2-(13Z,16Z-docosadienoyl)-glycero-3-phosphoserine; 1-docosanoyl-2-(7Z,10Z,13Z,16Z-docosatetraenoyl)-glycero-3-phosphoserine; 1-(11Z-docosenoyl)-glycero-3-phosphoserine; 1-(11Z-docosenoyl)-2-dodecanoyl-glycero-3-phosphoserine; 1-(11Z-docosenoyl)-2-tridecanoyl-glycero-3-phosphoserine; 1-(11Z-docosenoyl)-2-tetradecanoyl-glycero-3-phosphoserine; 1-(11Z-docosenoyl)-2-(9Z-tetradecenoyl)-glycero-3-phosphoserine; 1-(11Z-docosenoyl)-2-pentadecanoyl-glycero-3-phosphoserine; 1-(11Z-docosenoyl)-2-(9Z-pentadecenoyl)-glycero-3-phosphoserine; 1-(11Z-docosenoyl)-2-hexadecanoyl-glycero-3-phosphoserine; 1-(11Z-docosenoyl)-2-(9Z-hexadecenoyl)-glycero-3-phosphoserine; 1-(11Z-docosenoyl)-2-heptadecanoyl-glycero-3-phosphoserine; 1-(11Z-docosenoyl)-2-(9Z-heptadecenoyl)-glycero-3-phosphoserine; 1-(11Z-docosenoyl)-2-(9Z,12Z-heptadecadienoyl)-glycero-3-phosphoserine; 1-(11Z-docosenoyl)-2-octadecanoyl-glycero-3-phosphoserine; 1-(11Z-docosenoyl)-2-(9Z-octadecenoyl)-glycero-3-phosphoserine; 1-(11Z-docosenoyl)-2-(9Z,12Z-octadecadienoyl)-glycero-3-phosphoserine; 1-(11Z-docosenoyl)-2-(6Z,9Z,12Z-octadecatrienoyl)-glycero-3-phosphoserine; 1-(11Z-docosenoyl)-2-(9Z,12Z,15Z-octadecatrienoyl)-glycero-3-phosphoserine; 1-(11Z-docosenoyl)-2-(6Z,9Z,12Z,15Z-octadecatetraenoyl)-glycero-3-phosphoserine; 1-(11Z-docosenoyl)-2-nonadecanoyl-glycero-3-phosphoserine; 1-(11Z-docosenoyl)-2-(9Z-nonadecenoyl)-glycero-3-phosphoserine; 1-(11Z-docosenoyl)-2-eicosanoyl-glycero-3-phosphoserine; 1-(11Z-docosenoyl)-2-(11Z-eicosenoyl)-glycero-3-phosphoserine; 1-(11Z-docosenoyl)-2-(11Z,14Z-eicosadienoyl)-glycero-3-phosphoserine; 1-(11Z-docosenoyl)-2-(8Z,11Z,14Z-eicosatrienoyl)-glycero-3-phosphoserine; 1-(11Z-docosenoyl)-2-(5Z,8Z,11Z,14Z-eicosatetraenoyl)-glycero-3-phosphoserine; 1-(11Z-docosenoyl)-2-(5Z,8Z,11Z,14Z,17Z-eicosapentaenoyl)-glycero-3-phosphoserine; 1-(11Z-docosenoyl)-2-heneicosanoyl-glycero-3-phosphoserine; 1-(11Z-docosenoyl)-2-docosanoyl-glycero-3-phosphoserine; 1,2-di-(11Z-docosenoyl)-sn-glycero-3-phosphoserine; 1-(11Z-docosenoyl)-2-(13Z,16Z-docosadienoyl)-glycero-3-phosphoserine; 1-(11Z-docosenoyl)-2-(7Z,10Z,13Z,16Z-docosatetraenoyl)-glycero-3-phosphoserine; 1-(11Z-docosenoyl)-2-(4Z,7Z,10Z,13Z,16Z,19Z-docosahexaenoyl)-glycero-3-phosphoserine; 1-(13Z,16Z-docosadienoyl)-glycero-3-phosphoserine; 1-(13Z,16Z-docosadienoyl)-2-dodecanoyl-glycero-3-phosphoserine; 1-(13Z,16Z-docosadienoyl)-2-tridecanoyl-glycero-3-phosphoserine; 1-(13Z,16Z-docosadienoyl)-2-tetradecanoyl-glycero-3-phosphoserine; 1-(13Z,16Z-docosadienoyl)-2-(9Z-tetradecenoyl)-glycero-3-phosphoserine; 1-(13Z,16Z-docosadienoyl)-2-pentadecanoyl-glycero-3-phosphoserine; 1-(13Z,16Z-docosadienoyl)-2-(9Z-pentadecenoyl)-glycero-3-phosphoserine; 1-(13Z,16Z-docosadienoyl)-2-hexadecanoyl-glycero-3-phosphoserine; 1-(13Z,16Z-docosadienoyl)-2-(9Z-hexadecenoyl)-glycero-3-phosphoserine; 1-(13Z,16Z-docosadienoyl)-2-heptadecanoyl-glycero-3-phosphoserine; 1-(13Z,16Z-docosadienoyl)-2-(9Z-heptadecenoyl)-glycero-3-phosphoserine; 1-(13Z,16Z-docosadienoyl)-2-(9Z,12Z-heptadecadienoyl)-glycero-3-phosphoserine; 1-(13Z,16Z-docosadienoyl)-2-octadecanoyl-glycero-3-phosphoserine; 1-(13Z,16Z-docosadienoyl)-2-(9Z-octadecenoyl)-glycero-3-phosphoserine; 1-(13Z,16Z-docosadienoyl)-2-(9Z,12Z-octadecadienoyl)-glycero-3-phosphoserine; 1-(13Z,16Z-docosadienoyl)-2-(6Z,9Z,12Z-octadecatrienoyl)-glycero-3-phosphoserine; 1-(13Z,16Z-docosadienoyl)-2-(9Z,12Z,15Z-octadecatrienoyl)-glycero-3-phosphoserine; 1-(13Z,16Z-docosadienoyl)-2-(6Z,9Z,12Z,15Z-octadecatetraenoyl)-glycero-3-phosphoserine; 1-(13Z,16Z-docosadienoyl)-2-nonadecanoyl-glycero-3-phosphoserine; 1-(13Z,16Z-docosadienoyl)-2-(9Z-nonadecenoyl)-glycero-3-phosphoserine; 1-(13Z,16Z-docosadienoyl)-2-eicosanoyl-glycero-3-phosphoserine; 1-(13Z,16Z-docosadienoyl)-2-(11Z-eicosenoyl)-glycero-3-phosphoserine; 1-(13Z,16Z-docosadienoyl)-2-(11Z,14Z-eicosadienoyl)-glycero-3-phosphoserine; 1-(13Z,16Z-docosadienoyl)-2-(8Z,11Z,14Z-eicosatrienoyl)-glycero-3-phosphoserine; 1-(13Z,16Z-docosadienoyl)-2-(5Z,8Z,11Z,14Z-eicosatetraenoyl)-glycero-3-phosphoserine; 1-(13Z,16Z-docosadienoyl)-2-(5Z,8Z,11Z,14Z,17Z-eicosapentaenoyl)-glycero-3-phosphoserine; 1-(13Z,16Z-docosadienoyl)-2-heneicosanoyl-glycero-3-phosphoserine; 1-(13Z,16Z-docosadienoyl)-2-docosanoyl-glycero-3-phosphoserine; 1-(13Z,16Z-docosadienoyl)-2-(11Z-docosenoyl)-glycero-3-phosphoserine; 1,2-di-(13Z,16Z-docosadienoyl)-sn-glycero-3-phosphoserine; 1-(13Z,16Z-docosadienoyl)-2-(7Z,10Z,13Z,16Z-docosatetraenoyl)-glycero-3-phosphoserine; 1-(13Z,16Z-docosadienoyl)-2-(4Z,7Z,10Z,13Z,16Z,19Z-docosahexaenoyl)-glycero-3-phosphoserine; 1-(7Z,10Z,13Z,16Z-docosatetraenoyl)-2-dodecanoyl-glycero-3-phosphoserine; 1-(7Z,10Z,13Z,16Z-docosatetraenoyl)-2-tridecanoyl-glycero-3-phosphoserine; 1-(7Z,10Z,13Z,16Z-docosatetraenoyl)-2-tetradecanoyl-glycero-3-phosphoserine; 1-(7Z,10Z,13Z,16Z-docosatetraenoyl)-2-(9Z-tetradecenoyl)-glycero-3-phosphoserine; 1-(7Z,10Z,13Z,16Z-docosatetraenoyl)-2-pentadecanoyl-glycero-3-phosphoserine; 1-(7Z,10Z,13Z,16Z-docosatetraenoyl)-2-(9Z-pentadecenoyl)-glycero-3-phosphoserine; 1-(7Z,10Z,13Z,16Z-docosatetraenoyl)-2-hexadecanoyl-glycero-3-phosphoserine; 1-(7Z,10Z,13Z,16Z-docosatetraenoyl)-2-(9Z-hexadecenoyl)-glycero-3-phosphoserine; 1-(7Z,10Z,13Z,16Z-docosatetraenoyl)-2-heptadecanoyl-glycero-3-phosphoserine; 1-(7Z,10Z,13Z,16Z-docosatetraenoyl)-2-(9Z-heptadecenoyl)-glycero-3-phosphoserine; 1-(7Z,10Z,13Z,16Z-docosatetraenoyl)-2-(9Z,12Z-heptadecadienoyl)-glycero-3-phosphoserine; 1-(7Z,10Z,13Z,16Z-docosatetraenoyl)-2-octadecanoyl-glycero-3-phosphoserine; 1-(7Z,10Z,13Z,16Z-docosatetraenoyl)-2-(9Z-octadecenoyl)-glycero-3-phosphoserine; 1-(7Z,10Z,13Z,16Z-docosatetraenoyl)-2-(9Z,12Z-octadecadienoyl)-glycero-3-phosphoserine; 1-(7Z,10Z,13Z,16Z-docosatetraenoyl)-2-(6Z,9Z,12Z-octadecatrienoyl)-glycero-3-phosphoserine; 1-(7Z,10Z,13Z,16Z-docosatetraenoyl)-2-(9Z,12Z,15Z-octadecatrienoyl)-glycero-3-phosphoserine; 1-(7Z,10Z,13Z,16Z-docosatetraenoyl)-2-(6Z,9Z,12Z,15Z-octadecatetraenoyl)-glycero-3-phosphoserine; 1-(7Z,10Z,13Z,16Z-docosatetraenoyl)-2-nonadecanoyl-glycero-3-phosphoserine; 1-(7Z,10Z,13Z,16Z-docosatetraenoyl)-2-(9Z-nonadecenoyl)-glycero-3-phosphoserine; 1-(7Z,10Z,13Z,16Z-docosatetraenoyl)-2-eicosanoyl-glycero-3-phosphoserine; 1-(7Z,10Z,13Z,16Z-docosatetraenoyl)-2-(11Z-eicosenoyl)-glycero-3-phosphoserine; 1-(7Z,10Z,13Z,16Z-docosatetraenoyl)-2-(11Z,14Z-eicosadienoyl)-glycero-3-phosphoserine; 1-(7Z,10Z,13Z,16Z-docosatetraenoyl)-2-(8Z,11Z,14Z-eicosatrienoyl)-glycero-3-phosphoserine; 1-(7Z,10Z,13Z,16Z-docosatetraenoyl)-2-(5Z,8Z,11Z,14Z-eicosatetraenoyl)-glycero-3-phosphoserine; 1-(7Z,10Z,13Z,16Z-docosatetraenoyl)-2-(5Z,8Z,11Z,14Z,17Z-eicosapentaenoyl)-glycero-3-phosphoserine; 1-(7Z,10Z,13Z,16Z-docosatetraenoyl)-2-heneicosanoyl-glycero-3-phosphoserine; 1-(7Z,10Z,13Z,16Z-docosatetraenoyl)-2-docosanoyl-glycero-3-phosphoserine; 1-(7Z,10Z,13Z,16Z-docosatetraenoyl)-2-(11Z-docosenoyl)-glycero-3-phosphoserine; 1-(7Z,10Z,13Z,16Z-docosatetraenoyl)-2-(13Z,16Z-docosadienoyl)-glycero-3-phosphoserine; 1,2-di-(7Z,10Z,13Z,16Z-docosatetraenoyl)-sn-glycero-3-phosphoserine; 1-(4Z,7Z,10Z,13Z,16Z,19Z-docosahexaenoyl)-2-dodecanoyl-glycero-3-phosphoserine; 1-(4Z,7Z,10Z,13Z,16Z,19Z-docosahexaenoyl)-2-tridecanoyl-glycero-3-phosphoserine; 1-(4Z,7Z,10Z,13Z,16Z,19Z-docosahexaenoyl)-2-tetradecanoyl-glycero-3-phosphoserine; 1-(4Z,7Z,10Z,13Z,16Z,19Z-docosahexaenoyl)-2-(9Z-tetradecenoyl)-glycero-3-phosphoserine; 1-(4Z,7Z,10Z,13Z,16Z,19Z-docosahexaenoyl)-2-pentadecanoyl-glycero-3-phosphoserine; 1-(4Z,7Z,10Z,13Z,16Z,19Z-docosahexaenoyl)-2-(9Z-pentadecenoyl)-glycero-3-phosphoserine; 1-(4Z,7Z,10Z,13Z,16Z,19Z-docosahexaenoyl)-2-(9Z-hexadecenoyl)-glycero-3-phosphoserine; 1-(4Z,7Z,10Z,13Z,16Z,19Z-docosahexaenoyl)-2-heptadecanoyl-glycero-3-phosphoserine; 1-(4Z,7Z,10Z,13Z,16Z,19Z-docosahexaenoyl)-2-(9Z-heptadecenoyl)-glycero-3-phosphoserine; 1-(4Z,7Z,10Z,13Z,16Z,19Z-docosahexaenoyl)-2-(9Z,12Z-heptadecadienoyl)-glycero-3-phosphoserine; 1-(4Z,7Z,10Z,13Z,16Z,19Z-docosahexaenoyl)-2-octadecanoyl-glycero-3-phosphoserine; 1-(4Z,7Z,10Z,13Z,16Z,19Z-docosahexaenoyl)-2-(9Z-octadecenoyl)-glycero-3-phosphoserine; 1-(4Z,7Z,10Z,13Z,16Z,19Z-docosahexaenoyl)-2-(9Z,12Z-octadecadienoyl)-glycero-3-phosphoserine; 1-(4Z,7Z,10Z,13Z,16Z,19Z-docosahexaenoyl)-2-(6Z,9Z,12Z-octadecatrienoyl)-glycero-3-phosphoserine; 1-(4Z,7Z,10Z,13Z,16Z,19Z-docosahexaenoyl)-2-(9Z,12Z,15Z-octadecatrienoyl)-glycero-3-phosphoserine; 1-(4Z,7Z,10Z,13Z,16Z,19Z-docosahexaenoyl)-2-(6Z,9Z,12Z,15Z-octadecatetraenoyl)-glycero-3-phosphoserine; 1-(4Z,7Z,10Z,13Z,16Z,19Z-docosahexaenoyl)-2-nonadecanoyl-glycero-3-phosphoserine; 1-(4Z,7Z,10Z,13Z,16Z,19Z-docosahexaenoyl)-2-(9Z-nonadecenoyl)-glycero-3-phosphoserine; 1-(4Z,7Z,10Z,13Z,16Z,19Z-docosahexaenoyl)-2-eicosanoyl-glycero-3-phosphoserine; 1-(4Z,7Z,10Z,13Z,16Z,19Z-docosahexaenoyl)-2-(11Z-eicosenoyl)-glycero-3-phosphoserine; 1-(4Z,7Z,10Z,13Z,16Z,19Z-docosahexaenoyl)-2-(11Z,14Z-eicosadienoyl)-glycero-3-phosphoserine; 1-(4Z,7Z,10Z,13Z,16Z,19Z-docosahexaenoyl)-2-(8Z,11Z,14Z-eicosatrienoyl)-glycero-3-phosphoserine; 1-(4Z,7Z,10Z,13Z,16Z,19Z-docosahexaenoyl)-2-(5Z,8Z,11 Z,14Z-eicosatetraenoyl)-glycero-3-phosphoserine; 1-(4Z,7Z,10Z,13Z,16Z,19Z-docosahexaenoyl)-2-(5Z,8Z,11Z,14Z,17Z-eicosapentaenoyl)-glycero-3-phosphoserine; 1-(4Z,7Z,10Z,13Z,16Z,19Z-docosahexaenoyl)-2-heneicosanoyl-glycero-3-phosphoserine; 1-(4Z,7Z,10Z,13Z,16Z,19Z-docosahexaenoyl)-2-docosanoyl-glycero-3-phosphoserine; 1-(4Z,7Z,10Z,13Z,16Z,19Z-docosahexaenoyl)-2-(11Z-docosenoyl)-glycero-3-phosphoserine; 1-(4Z,7Z,10Z,13Z,16Z,19Z-docosahexaenoyl)-2-(13Z,16Z-docosadienoyl)-glycero-3-phosphoserine; 1-(4Z,7Z,10Z,13Z,16Z,19Z-docosahexaenoyl)-2-(7Z,10Z,13Z,16Z-docosatetraenoyl)-glycero-3-phosphoserine; 1-hexadecyl-2-dodecanoyl-glycero-3-phosphoserine; 1-hexadecyl-2-tridecanoyl-glycero-3-phosphoserine; 1-hexadecyl-2-tetradecanoyl-glycero-3-phosphoserine; 1-hexadecyl-2-(9Z-tetradecenoyl)-glycero-3-phosphoserine; 1-hexadecyl-2-pentadecanoyl-glycero-3-phosphoserine; 1-hexadecyl-2-(9Z-pentadecenoyl)-glycero-3-phosphoserine; 1-hexadecyl-2-(9Z-hexadecenoyl)-glycero-3-phosphoserine; 1-hexadecyl-2-(9Z-heptadecenoyl)-glycero-3-phosphoserine; 1-hexadecyl-2-(9Z,12Z-heptadecadienoyl)-glycero-3-phosphoserine; 1-hexadecyl-2-(6Z,9Z,12Z,15Z-octadecatetraenoyl)-glycero-3-phosphoserine; 1-hexadecyl-2-(9Z-nonadecenoyl)-glycero-3-phosphoserine; 1-hexadecyl-2-(11Z-eicosenoyl)-glycero-3-phosphoserine; 1-hexadecyl-2-(11Z,14Z-eicosadienoyl)-glycero-3-phosphoserine; 1-hexadecyl-2-heneicosanoyl-glycero-3-phosphoserine; 1-hexadecyl-2-(11Z-docosenoyl)-glycero-3-phosphoserine; 1-hexadecyl-2-(13Z,16Z-docosadienoyl)-glycero-3-phosphoserine; 1-hexadecyl-2-(7Z,10Z,13Z,16Z-docosatetraenoyl)-glycero-3-phosphoserine; 1-octadecyl-2-dodecanoyl-glycero-3-phosphoserine; 1-octadecyl-2-tridecanoyl-glycero-3-phosphoserine; 1-octadecyl-2-tetradecanoyl-glycero-3-phosphoserine; 1-octadecyl-2-(9Z-tetradecenoyl)-glycero-3-phosphoserine; 1-octadecyl-2-pentadecanoyl-glycero-3-phosphoserine; 1-octadecyl-2-(9Z-pentadecenoyl)-glycero-3-phosphoserine; 1-octadecyl-2-heptadecanoyl-glycero-3-phosphoserine; 1-octadecyl-2-(9Z-heptadecenoyl)-glycero-3-phosphoserine; 1-octadecyl-2-(9Z,12Z-heptadecadienoyl)-glycero-3-phosphoserine; 1-octadecyl-2-(9Z-octadecenoyl)-glycero-3-phosphoserine; 1-octadecyl-2-(9Z,12Z-octadecadienoyl)-glycero-3-phosphoserine; 1-octadecyl-2-(6Z,9Z,12Z-octadecatrienoyl)-glycero-3-phosphoserine; 1-octadecyl-2-(9Z,12Z,15Z-octadecatrienoyl)-glycero-3-phosphoserine; 1-octadecyl-2-(6Z,9Z,12Z,15Z-octadecatetraenoyl)-glycero-3-phosphoserine; 1-octadecyl-2-nonadecanoyl-glycero-3-phosphoserine; 1-octadecyl-2-(9Z-nonadecenoyl)-glycero-3-phosphoserine; 1-octadecyl-2-(11Z-eicosenoyl)-glycero-3-phosphoserine; 1-octadecyl-2-(11Z,14Z-eicosadienoyl)-glycero-3-phosphoserine; 1-octadecyl-2-(8Z,11Z,14Z-eicosatrienoyl)-glycero-3-phosphoserine; 1-octadecyl-2-heneicosanoyl-glycero-3-phosphoserine; 1-octadecyl-2-(11Z-docosenoyl)-glycero-3-phosphoserine; 1-octadecyl-2-(13Z,16Z-docosadienoyl)-glycero-3-phosphoserine; 1-octadecyl-2-(7Z,10Z,13Z,16Z-docosatetraenoyl)-glycero-3-phosphoserine; 1-eicosyl-glycero-3-phosphoserine; 1-eicosyl-2-dodecanoyl-glycero-3-phosphoserine; 1-eicosyl-2-tridecanoyl-glycero-3-phosphoserine; 1-eicosyl-2-tetradecanoyl-glycero-3-phosphoserine; 1-eicosyl-2-(9Z-tetradecenoyl)-glycero-3-phosphoserine; 1-eicosyl-2-pentadecanoyl-glycero-3-phosphoserine; 1-eicosyl-2-(9Z-pentadecenoyl)-glycero-3-phosphoserine; 1-eicosyl-2-(9Z-hexadecenoyl)-glycero-3-phosphoserine; 1-eicosyl-2-heptadecanoyl-glycero-3-phosphoserine; 1-eicosyl-2-(9Z-heptadecenoyl)-glycero-3-phosphoserine; 1-eicosyl-2-(9Z,12Z-heptadecadienoyl)-glycero-3-phosphoserine; 1-eicosyl-2-octadecanoyl-glycero-3-phosphoserine; 1-eicosyl-2-(9Z-octadecenoyl)-glycero-3-phosphoserine; 1-eicosyl-2-(9Z,12Z-octadecadienoyl)-glycero-3-phosphoserine; 1-eicosyl-2-(6Z,9Z,12Z-octadecatrienoyl)-glycero-3-phosphoserine; 1-eicosyl-2-(9Z,12Z,15Z-octadecatrienoyl)-glycero-3-phosphoserine; 1-eicosyl-2-(6Z,9Z,12Z,15Z-octadecatetraenoyl)-glycero-3-phosphoserine; 1-eicosyl-2-nonadecanoyl-glycero-3-phosphoserine; 1-eicosyl-2-(9Z-nonadecenoyl)-glycero-3-phosphoserine; 1-eicosyl-2-eicosanoyl-glycero-3-phosphoserine; 1-eicosyl-2-(11Z-eicosenoyl)-glycero-3-phosphoserine; 1-eicosyl-2-(11Z,14Z-eicosadienoyl)-glycero-3-phosphoserine; 1-eicosyl-2-(8Z,11Z,14Z-eicosatrienoyl)-glycero-3-phosphoserine; 1-eicosyl-2-(5Z,8Z,11Z,14Z-eicosatetraenoyl)-glycero-3-phosphoserine; 1-eicosyl-2-(5Z,8Z,11Z,14Z,17Z-eicosapentaenoyl)-glycero-3-phosphoserine; 1-eicosyl-2-heneicosanoyl-glycero-3-phosphoserine; 1-eicosyl-2-(11Z-docosenoyl)-glycero-3-phosphoserine; 1-eicosyl-2-(13Z,16Z-docosadienoyl)-glycero-3-phosphoserine; 1-eicosyl-2-(7Z,10Z,13Z,16Z-docosatetraenoyl)-glycero-3-phosphoserine; 1-(1Z-hexadecenyl)-2-dodecanoyl-glycero-3-phosphoserine; 1-(1Z-hexadecenyl)-2-tridecanoyl-glycero-3-phosphoserine; 1-(1Z-hexadecenyl)-2-tetradecanoyl-glycero-3-phosphoserine; 1-(1Z-hexadecenyl)-2-(9Z-tetradecenoyl)-glycero-3-phosphoserine; 1-(1Z-hexadecenyl)-2-pentadecanoyl-glycero-3-phosphoserine; 1-(1Z-hexadecenyl)-2-(9Z-pentadecenoyl)-glycero-3-phosphoserine; 1-(1Z-hexadecenyl)-2-hexadecanoyl-glycero-3-phosphoserine; 1-(1Z-hexadecenyl)-2-(9Z-hexadecenoyl)-glycero-3-phosphoserine; 1-(1Z-hexadecenyl)-2-heptadecanoyl-glycero-3-phosphoserine; 1-(1Z-hexadecenyl)-2-(9Z-heptadecenoyl)-glycero-3-phosphoserine; 1-(1Z-hexadecenyl)-2-(9Z,12Z-heptadecadienoyl)-glycero-3-phosphoserine; 1-(1Z-hexadecenyl)-2-octadecanoyl-glycero-3-phosphoserine; 1-(1Z-hexadecenyl)-2-(6Z,9Z,12Z-octadecatrienoyl)-glycero-3-phosphoserine; 1-(1Z-hexadecenyl)-2-(9Z,12Z,15Z-octadecatrienoyl)-glycero-3-phosphoserine; 1-(1Z-hexadecenyl)-2-(6Z,9Z,12Z,15Z-octadecatetraenoyl)-glycero-3-phosphoserine; 1-(1Z-hexadecenyl)-2-nonadecanoyl-glycero-3-phosphoserine; 1-(1Z-hexadecenyl)-2-(9Z-nonadecenoyl)-glycero-3-phosphoserine; 1-(1Z-hexadecenyl)-2-eicosanoyl-glycero-3-phosphoserine; 1-(1Z-hexadecenyl)-2-(11Z-eicosenoyl)-glycero-3-phosphoserine; 1-(1Z-hexadecenyl)-2-(11Z,14Z-eicosadienoyl)-glycero-3-phosphoserine; 1-(1Z-hexadecenyl)-2-(8Z,11Z,14Z-eicosatrienoyl)-glycero-3-phosphoserine; 1-(1Z-hexadecenyl)-2-(5Z,8Z,11Z,14Z,17Z-eicosapentaenoyl)-glycero-3-phosphoserine; 1-(1Z-hexadecenyl)-2-heneicosanoyl-glycero-3-phosphoserine; 1-(1Z-hexadecenyl)-2-docosanoyl-glycero-3-phosphoserine; 1-(1Z-hexadecenyl)-2-(11Z-docosenoyl)-glycero-3-phosphoserine; 1-(1Z-hexadecenyl)-2-(13Z,16Z-docosadienoyl)-glycero-3-phosphoserine; 1-(1Z-hexadecenyl)-2-(7Z,10Z,13Z,16Z-docosatetraenoyl)-glycero-3-phosphoserine; 1-(1Z-octadecenyl)-2-dodecanoyl-glycero-3-phosphoserine; 1-(1Z-octadecenyl)-2-tridecanoyl-glycero-3-phosphoserine; 1-(1Z-octadecenyl)-2-tetradecanoyl-glycero-3-phosphoserine; 1-(1Z-octadecenyl)-2-(9Z-tetradecenoyl)-glycero-3-phosphoserine; 1-(1Z-octadecenyl)-2-pentadecanoyl-glycero-3-phosphoserine; 1-(1Z-octadecenyl)-2-(9Z-pentadecenoyl)-glycero-3-phosphoserine; 1-(1Z-octadecenyl)-2-hexadecanoyl-glycero-3-phosphoserine; 1-(1Z-octadecenyl)-2-(9Z-hexadecenoyl)-glycero-3-phosphoserine; 1-(1Z-octadecenyl)-2-heptadecanoyl-glycero-3-phosphoserine; 1-(1Z-octadecenyl)-2-(9Z-heptadecenoyl)-glycero-3-phosphoserine; 1-(1Z-octadecenyl)-2-(9Z,12Z-heptadecadienoyl)-glycero-3-phosphoserine; 1-(1Z-octadecenyl)-2-octadecanoyl-glycero-3-phosphoserine; 1-(1Z-octadecenyl)-2-(9Z,12Z-octadecadienoyl)-glycero-3-phosphoserine; 1-(1Z-octadecenyl)-2-(6Z,9Z,12Z-octadecatrienoyl)-glycero-3-phosphoserine; 1-(1Z-octadecenyl)-2-(9Z,12Z,15Z-octadecatrienoyl)-glycero-3-phosphoserine; 1-(1Z-octadecenyl)-2-(6Z,9Z,12Z,15Z-octadecatetraenoyl)-glycero-3-phosphoserine; 1-(1Z-octadecenyl)-2-nonadecanoyl-glycero-3-phosphoserine; 1-(1Z-octadecenyl)-2-(9Z-nonadecenoyl)-glycero-3-phosphoserine; 1-(1Z-octadecenyl)-2-eicosanoyl-glycero-3-phosphoserine; 1-(1Z-octadecenyl)-2-(11Z-eicosenoyl)-glycero-3-phosphoserine; 1-(1Z-octadecenyl)-2-(11Z,14Z-eicosadienoyl)-glycero-3-phosphoserine; 1-(1Z-octadecenyl)-2-(8Z,11Z,14Z-eicosatrienoyl)-glycero-3-phosphoserine; 1-(1Z-octadecenyl)-2-(5Z,8Z,11Z,14Z,17Z-eicosapentaenoyl)-glycero-3-phosphoserine; 1-(1Z-octadecenyl)-2-heneicosanoyl-glycero-3-phosphoserine; 1-(1Z-octadecenyl)-2-docosanoyl-glycero-3-phosphoserine; 1-(1Z-octadecenyl)-2-(11Z-docosenoyl)-glycero-3-phosphoserine; 1-(1Z-octadecenyl)-2-(13Z,16Z-docosadienoyl)-glycero-3-phosphoserine; 1-(1Z-octadecenyl)-2-(7Z,10Z,13Z,16Z-docosatetraenoyl)-glycero-3-phosphoserine; 1-(1Z-eicosenyl)-glycero-3-phosphoserine; 1-(1Z-eicosenyl)-2-dodecanoyl-glycero-3-phosphoserine; 1-(1Z-eicosenyl)-2-tridecanoyl-glycero-3-phosphoserine; 1-(1Z-eicosenyl)-2-tetradecanoyl-glycero-3-phosphoserine; 1-(1Z-eicosenyl)-2-(9Z-tetradecenoyl)-glycero-3-phosphoserine; 1-(1Z-eicosenyl)-2-pentadecanoyl-glycero-3-phosphoserine; 1-(1Z-eicosenyl)-2-(9Z-pentadecenoyl)-glycero-3-phosphoserine; 1-(1Z-eicosenyl)-2-hexadecanoyl-glycero-3-phosphoserine; 1-(1Z-eicosenyl)-2-(9Z-hexadecenoyl)-glycero-3-phosphoserine; 1-(1Z-eicosenyl)-2-heptadecanoyl-glycero-3-phosphoserine; 1-(1Z-eicosenyl)-2-(9Z-heptadecenoyl)-glycero-3-phosphoserine; 1-(1Z-eicosenyl)-2-(9Z,12Z-heptadecadienoyl)-glycero-3-phosphoserine; 1-(1Z-eicosenyl)-2-octadecanoyl-glycero-3-phosphoserine; 1-(1Z-eicosenyl)-2-(9Z-octadecenoyl)-glycero-3-phosphoserine; 1-(1Z-eicosenyl)-2-(9Z,12Z-octadecadienoyl)-glycero-3-phosphoserine; 1-(1Z-eicosenyl)-2-(6Z,9Z,12Z-octadecatrienoyl)-glycero-3-phosphoserine; 1-(1Z-eicosenyl)-2-(9Z,12Z,15Z-octadecatrienoyl)-glycero-3-phosphoserine; 1-(1Z-eicosenyl)-2-(6Z,9Z,12Z,15Z-octadecatetraenoyl)-glycero-3-phosphoserine; 1-(1Z-eicosenyl)-2-nonadecanoyl-glycero-3-phosphoserine; 1-(1Z-eicosenyl)-2-(9Z-nonadecenoyl)-glycero-3-phosphoserine; 1-(1Z-eicosenyl)-2-eicosanoyl-glycero-3-phosphoserine; 1-(1Z-eicosenyl)-2-(11Z-eicosenoyl)-glycero-3-phosphoserine; 1-(1Z-eicosenyl)-2-(11Z,14Z-eicosadienoyl)-glycero-3-phosphoserine; 1-(1Z-eicosenyl)-2-(8Z,11Z,14Z-eicosatrienoyl)-glycero-3-phosphoserine; 1-(1Z-eicosenyl)-2-(5Z,8Z,11Z,14Z-eicosatetraenoyl)-glycero-3-phosphoserine; 1-(1Z-eicosenyl)-2-(5Z,8Z,11Z,14Z,17Z-eicosapentaenoyl)-glycero-3-phosphoserine; 1-(1Z-eicosenyl)-2-heneicosanoyl-glycero-3-phosphoserine; 1-(1Z-eicosenyl)-2-docosanoyl-glycero-3-phosphoserine; 1-(1Z-eicosenyl)-2-(11Z-docosenoyl)-glycero-3-phosphoserine; 1-(1Z-eicosenyl)-2-(13Z,16Z-docosadienoyl)-glycero-3-phosphoserine; 1-(1Z-eicosenyl)-2-(7Z,10Z,13Z,16Z-docosatetraenoyl)-glycero-3-phosphoserine; 1-(1Z-hexadecenyl)-2-(5Z,8Z,11Z,14Z-eicosatetraenoyl)-glycero-3-phosphoserine; 1-(1Z-hexadecenyl)-2-(9Z,12Z-octadecadienoyl)-glycero-3-phosphoserine; 1-(1Z-hexadecenyl)-2-(9Z-octadecenoyl)-glycero-3-phosphoserine; 1-eicosyl-2-docosanoyl-glycero-3-phosphoserine; 1-eicosyl-2-hexadecanoyl-glycero-3-phosphoserine; 1-octadecyl-2-(5Z,8Z,11Z,14Z-eicosatetraenoyl)-glycero-3-phosphoserine; 1-octadecyl-2-(9Z-hexadecenoyl)-glycero-3-phosphoserine; 1-octadecyl-2-hexadecanoyl-glycero-3-phosphoserine; 1-hexadecyl-2-(5Z,8Z,11Z,14Z,17Z-eicosapentaenoyl)-glycero-3-phosphoserine; 1-hexadecyl-2-(8Z,11Z,14Z-eicosatrienoyl)-glycero-3-phosphoserine; 1-hexadecyl-2-nonadecanoyl-glycero-3-phosphoserine; 1-hexadecyl-2-(9Z,12Z,15Z-octadecatrienoyl)-glycero-3-phosphoserine; 1-hexadecyl-2-(6Z,9Z,12Z-octadecatrienoyl)-glycero-3-phosphoserine; 1-hexadecyl-2-(9Z,12Z-octadecadienoyl)-glycero-3-phosphoserine; 1-hexadecyl-2-octadecanoyl-glycero-3-phosphoserine; 1-hexadecyl-2-heptadecanoyl-glycero-3-phosphoserine; 1-hexadecyl-2-hexadecanoyl-glycero-3-phosphoserine; 1-(4Z,7Z,10Z,13Z,16Z,19Z-docosahexaenoyl)-2-hexadecanoyl-glycero-3-phosphoserine; 1-docosanoyl-2-octadecanoyl-glycero-3-phosphoserine; 1-docosanoyl-2-tetradecanoyl-glycero-3-phosphoserine; 1-docosanoyl-2-tridecanoyl-glycero-3-phosphoserine; 1-docosanoyl-2-dodecanoyl-glycero-3-phosphoserine; 1-docosanoyl-glycero-3-phosphoserine; 1,2-diheneicosanoyl-sn-glycero-3-phosphoserine; 1-heneicosanoyl-2-octadecanoyl-glycero-3-phosphoserine; 1-heneicosanoyl-2-tetradecanoyl-glycero-3-phosphoserine; 1-heneicosanoyl-2-tridecanoyl-glycero-3-phosphoserine; 1-heneicosanoyl-2-dodecanoyl-glycero-3-phosphoserine; 1-heneicosanoyl-glycero-3-phosphoserine; 1-(5Z,8Z,11Z,14Z,17Z-eicosapentaenoyl)-2-(4Z,7Z,10Z,13Z,16Z,19Z-docosahexaenoyl)-glycero-3-phosphoserine; 1-(5Z,8Z,11Z,14Z,17Z-eicosapentaenoyl)-2-(9Z-octadecenoyl)-glycero-3-phosphoserine; 1-(5Z,8Z,11Z,14Z,17Z-eicosapentaenoyl)-glycero-3-phosphoserine; 1,2-di-(5Z,8Z,11Z,14Z-eicosatetraenoyl)-sn-glycero-3-phosphoserine; 1-(5Z,8Z,11Z,14Z-eicosatetraenoyl)-2-hexadecanoyl-glycero-3-phosphoserine; 1,2-di-(11Z-eicosenoyl)-sn-glycero-3-phosphoserine; 1-eicosanoyl-2-(4Z,7Z,10Z,13Z,16Z,19Z-docosahexaenoyl)-glycero-3-phosphoserine; 1-eicosanoyl-2-(5Z,8Z,11Z,14Z-eicosatetraenoyl)-glycero-3-phosphoserine; 1,2-dieicosanoyl-sn-glycero-3-phosphoserine; 1-eicosanoyl-2-hexadecanoyl-glycero-3-phosphoserine; 1-eicosanoyl-2-tetradecanoyl-glycero-3-phosphoserine; 1-eicosanoyl-2-tridecanoyl-glycero-3-phosphoserine; 1-eicosanoyl-2-dodecanoyl-glycero-3-phosphoserine; 1,2-dinonadecanoyl-sn-glycero-3-phosphoserine; 1-nonadecanoyl-2-octadecanoyl-glycero-3-phosphoserine; 1-nonadecanoyl-2-pentadecanoyl-glycero-3-phosphoserine; 1-nonadecanoyl-2-tetradecanoyl-glycero-3-phosphoserine; 1-nonadecanoyl-2-tridecanoyl-glycero-3-phosphoserine; 1-nonadecanoyl-2-dodecanoyl-glycero-3-phosphoserine; 1-nonadecanoyl-glycero-3-phosphoserine; 1-(9Z,12Z,15Z-octadecatrienoyl)-2-(9Z-octadecenoyl)-glycero-3-phosphoserine; 1-(9Z,12Z,15Z-octadecatrienoyl)-glycero-3-phosphoserine; 1-(9Z,12Z-octadecadienoyl)-2-(5Z,8Z,11Z,14Z-eicosatetraenoyl)-glycero-3-phosphoserine; 1-(9Z,12Z-octadecadienoyl)-2-octadecanoyl-glycero-3-phosphoserine; 1-(9Z,12Z-octadecadienoyl)-2-hexadecanoyl-glycero-3-phosphoserine; 1-(9Z-octadecenoyl)-2-(5Z,8Z,11Z,14Z,17Z-eicosapentaenoyl)-glycero-3-phosphoserine; 1-(9Z-octadecenoyl)-2-(5Z,8Z,11Z,14Z-eicosatetraenoyl)-glycero-3-phosphoserine; 1-(9Z-octadecenoyl)-2-(9Z,12Z,15Z-octadecatrienoyl)-glycero-3-phosphoserine; 1-(9Z-octadecenoyl)-2-(9Z-hexadecenoyl)-glycero-3-phosphoserine; 1-(9Z-octadecenoyl)-2-tetradecanoyl-glycero-3-phosphoserine; 1-octadecanoyl-2-(7Z,10Z,13Z,16Z-docosatetraenoyl)-glycero-3-phosphoserine; 1-octadecanoyl-2-(5Z,8Z,11Z,14Z,17Z-eicosapentaenoyl)-glycero-3-phosphoserine; 1-octadecanoyl-2-(8Z,11Z,14Z-eicosatrienoyl)-glycero-3-phosphoserine; 1-octadecanoyl-2-(6Z,9Z,12Z,15Z-octadecatetraenoyl)-glycero-3-phosphoserine; 1-octadecanoyl-2-(9Z-hexadecenoyl)-glycero-3-phosphoserine; 1-octadecanoyl-2-hexadecanoyl-glycero-3-phosphoserine; 1-octadecanoyl-2-dodecanoyl-glycero-3-phosphoserine; 1,2-di-(9Z,12Z-heptadecadienoyl)-sn-glycero-3-phosphoserine; 1,2-di-(9Z-heptadecenoyl)-sn-glycero-3-phosphoserine; 1-heptadecanoyl-2-(4Z,7Z,10Z,13Z,16Z,19Z-docosahexaenoyl)-glycero-3-phosphoserine; 1-heptadecanoyl-2-(9Z-octadecenoyl)-glycero-3-phosphoserine; 1-heptadecanoyl-2-octadecanoyl-glycero-3-phosphoserine; 1,2-diheptadecanoyl-sn-glycero-3-phosphoserine; 1-heptadecanoyl-2-pentadecanoyl-glycero-3-phosphoserine; 1-heptadecanoyl-2-tridecanoyl-glycero-3-phosphoserine; 1-heptadecanoyl-glycero-3-phosphoserine; 1-(9Z-hexadecenoyl)-2-(5Z,8Z,11Z,14Z-eicosatetraenoyl)-glycero-3-phosphoserine; 1-(9Z-hexadecenoyl)-2-(6Z,9Z,12Z,15Z-octadecatetraenoyl)-glycero-3-phosphoserine; 1-(9Z-hexadecenoyl)-2-(9Z,12Z-octadecadienoyl)-glycero-3-phosphoserine; 1-(9Z-hexadecenoyl)-2-(9Z-octadecenoyl)-glycero-3-phosphoserine; 1-hexadecanoyl-2-(5Z,8Z,11Z,14Z,17Z-eicosapentaenoyl)-glycero-3-phosphoserine; 1-hexadecanoyl-2-(8Z,11Z,14Z-eicosatrienoyl)-glycero-3-phosphoserine; 1-hexadecanoyl-2-(6Z,9Z,12Z,15Z-octadecatetraenoyl)-glycero-3-phosphoserine; 1-hexadecanoyl-2-(6Z,9Z,12Z-octadecatrienoyl)-glycero-3-phosphoserine; 1-hexadecanoyl-2-octadecanoyl-glycero-3-phosphoserine; 1-hexadecanoyl-2-(9Z-heptadecenoyl)-glycero-3-phosphoserine; 1-hexadecanoyl-2-heptadecanoyl-glycero-3-phosphoserine; 1-hexadecanoyl-2-(9Z-hexadecenoyl)-glycero-3-phosphoserine; 1-hexadecanoyl-2-pentadecanoyl-glycero-3-phosphoserine; 1-pentadecanoyl-2-heneicosanoyl-glycero-3-phosphoserine; 1-pentadecanoyl-2-nonadecanoyl-glycero-3-phosphoserine; 1-pentadecanoyl-2-(9Z,12Z-octadecadienoyl)-glycero-3-phosphoserine; 1-pentadecanoyl-2-octadecanoyl-glycero-3-phosphoserine; 1-pentadecanoyl-2-heptadecanoyl-glycero-3-phosphoserine; 1-pentadecanoyl-2-(9Z-hexadecenoyl)-glycero-3-phosphoserine; 1-pentadecanoyl-2-hexadecanoyl-glycero-3-phosphoserine; 1-pentadecanoyl-2-tridecanoyl-glycero-3-phosphoserine; 1-pentadecanoyl-glycero-3-phosphoserine; 1,2-di-(9Z-tetradecenoyl)-sn-glycero-3-phosphoserine; 1-(9Z-tetradecenoyl)-glycero-3-phosphoserine; 1-tetradecanoyl-2-(4Z,7Z,10Z,13Z,16Z,19Z-docosahexaenoyl)-glycero-3-phosphoserine; 1-tetradecanoyl-2-docosanoyl-glycero-3-phosphoserine; 1-tetradecanoyl-2-(5Z,8Z,11Z,14Z,17Z-eicosapentaenoyl)-glycero-3-phosphoserine; 1-tetradecanoyl-2-(5Z,8Z,11Z,14Z-eicosatetraenoyl)-glycero-3-phosphoserine; 1-tetradecanoyl-2-eicosanoyl-glycero-3-phosphoserine; 1-tetradecanoyl-2-(6Z,9Z,12Z,15Z-octadecatetraenoyl)-glycero-3-phosphoserine; 1-tetradecanoyl-2-(9Z,12Z,15Z-octadecatrienoyl)-glycero-3-phosphoserine; 1-tetradecanoyl-2-(9Z,12Z-octadecadienoyl)-glycero-3-phosphoserine; 1-tetradecanoyl-2-octadecanoyl-glycero-3-phosphoserine; 1-tetradecanoyl-2-(9Z-hexadecenoyl)-glycero-3-phosphoserine; 1-tetradecanoyl-2-pentadecanoyl-glycero-3-phosphoserine; 1-tetradecanoyl-2-dodecanoyl-glycero-3-phosphoserine; 1-tridecanoyl-2-heneicosanoyl-glycero-3-phosphoserine; 1-tridecanoyl-2-eicosanoyl-glycero-3-phosphoserine; 1-tridecanoyl-2-nonadecanoyl-glycero-3-phosphoserine; 1-tridecanoyl-2-heptadecanoyl-glycero-3-phosphoserine; 1-tridecanoyl-2-pentadecanoyl-glycero-3-phosphoserine; 1-dodecanoyl-2-(4Z,7Z,10Z,13Z,16Z,19Z-docosahexaenoyl)-glycero-3-phosphoserine; 1-dodecanoyl-2-docosanoyl-glycero-3-phosphoserine; 1-dodecanoyl-2-eicosanoyl-glycero-3-phosphoserine; 1-dodecanoyl-2-(9Z-octadecenoyl)-glycero-3-phosphoserine; 1-dodecanoyl-2-octadecanoyl-glycero-3-phosphoserine; 1-dodecanoyl-2-hexadecanoyl-glycero-3-phosphoserine; 1-(1Z-octadecenyl)-2-(4Z,7Z,10Z,13Z,16Z,19Z-docosahexaenoyl)-glycero-3-phosphoserine; 1-(1Z-octadecenyl)-2-(5Z,8Z,11Z,14Z-eicosatetraenoyl)-glycero-3-phosphoserine; 1-(1Z-octadecenyl)-2-(9Z-octadecenoyl)-glycero-3-phosphoserine; 1-(1Z-octadecenyl)-glycero-3-phosphoserine; 1-(1Z-hexadecenyl)-2-(4Z,7Z,10Z,13Z,16Z,19Z-docosahexaenoyl)-glycero-3-phosphoserine; 1-(1Z-hexadecenyl)-glycero-3-phosphoserine; 1-octadecyl-2-(4Z,7Z,10Z,13Z,16Z,19Z-docosahexaenoyl)-glycero-3-phosphoserine; 1-octadecyl-2-docosanoyl-glycero-3-phosphoserine; 1-octadecyl-2-(5Z,8Z,11Z,14Z,17Z-eicosapentaenoyl)-glycero-3-phosphoserine; 1-octadecyl-2-eicosanoyl-glycero-3-phosphoserine; 1-octadecyl-2-octadecanoyl-glycero-3-phosphoserine; 1-octadecyl-glycero-3-phosphoserine; 1-hexadecyl-2-(4Z,7Z,10Z,13Z,16Z,19Z-docosahexaenoyl)-glycero-3-phosphoserine; 1-hexadecyl-2-docosanoyl-glycero-3-phosphoserine; 1-hexadecyl-2-(5Z,8Z,11Z,14Z-eicosatetraenoyl)-glycero-3-phosphoserine; 1-hexadecyl-2-eicosanoyl-glycero-3-phosphoserine; 1-hexadecyl-2-(9Z-octadecenoyl)-glycero-3-phosphoserine; 1-hexadecyl-glycero-3-phosphoserine; 1,2-didocosanoyl-sn-glycero-3-phosphoserine; 1-docosanoyl-2-(11Z-eicosenoyl)-glycero-3-phosphoserine; 1-docosanoyl-2-(9Z-octadecenoyl)-glycero-3-phosphoserine; 1-eicosanoyl-2-docosanoyl-glycero-3-phosphoserine; 1-eicosanoyl-2-(11Z,14Z-eicosadienoyl)-glycero-3-phosphoserine; 1-eicosanoyl-2-(11Z-eicosenoyl)-glycero-3-phosphoserine; 1-eicosanoyl-2-(6Z,9Z,12Z,15Z-octadecatetraenoyl)-glycero-3-phosphoserine; 1-eicosanoyl-2-(9Z,12Z-octadecadienoyl)-glycero-3-phosphoserine; 1-eicosanoyl-2-(9Z-octadecenoyl)-glycero-3-phosphoserine; 1-eicosanoyl-2-octadecanoyl-glycero-3-phosphoserine; 1-nonadecanoyl-2-hexadecanoyl-glycero-3-phosphoserine; 1-(9Z,12Z,15Z-octadecatrienoyl)-2-hexadecanoyl-glycero-3-phosphoserine; 1-(9Z-octadecenoyl)-2-docosanoyl-glycero-3-phosphoserine; 1-(9Z-octadecenoyl)-2-(11 Z-eicosenoyl)-glycero-3-phosphoserine; 1-(9Z-octadecenoyl)-2-eicosanoyl-glycero-3-phosphoserine; 1-(9Z-octadecenoyl)-2-(9Z,12Z-octadecadienoyl)-glycero-3-phosphoserine; 1-(9Z-octadecenoyl)-2-hexadecanoyl-glycero-3-phosphoserine; 1-octadecanoyl-2-(11Z,14Z-eicosadienoyl)-glycero-3-phosphoserine; 1-octadecanoyl-2-(11Z-eicosenoyl)-glycero-3-phosphoserine; 1-octadecanoyl-2-eicosanoyl-glycero-3-phosphoserine; 1-heptadecanoyl-2-eicosanoyl-glycero-3-phosphoserine; 1,2-di-(9Z-hexadecenoyl)-sn-glycero-3-phosphoserine; 1-hexadecanoyl-2-(7Z,10Z,13Z,16Z-docosatetraenoyl)-glycero-3-phosphoserine; 1-hexadecanoyl-2-docosanoyl-glycero-3-phosphoserine; 1-hexadecanoyl-2-(11Z-eicosenoyl)-glycero-3-phosphoserine; 1-hexadecanoyl-2-nonadecanoyl-glycero-3-phosphoserine; 1-hexadecanoyl-2-(9Z,12Z,15Z-octadecatrienoyl)-glycero-3-phosphoserine; 1-hexadecanoyl-2-tetradecanoyl-glycero-3-phosphoserine; 1,2-ditridecanoyl-sn-glycero-3-phosphoserine; 1-dodecanoyl-2-tetradecanoyl-glycero-3-phosphoserine; 1-docosanoyl-2-(4Z,7Z,10Z,13Z,16Z,19Z-docosahexaenoyl)-glycero-3-phosphoserine; 1,2-di-(9Z,12Z,15Z-octadecatrienoyl)-sn-glycero-3-phosphoserine; 1-octadecanoyl-2-docosanoyl-glycero-3-phosphoserine; 1-(9Z-heptadecenoyl)-glycero-3-phosphoserine; 1-hexadecanoyl-2-(9Z,12Z-octadecadienoyl)-glycero-3-phosphoserine; 1-tetradecanoyl-2-(9Z-octadecenoyl)-glycero-3-phosphoserine; 1-(1Z-eicosenyl)-2-(4Z,7Z,10Z,13Z,16Z,19Z-docosahexaenoyl)-glycero-3-phosphoserine; 1-eicosyl-2-(4Z,7Z,10Z,13Z,16Z,19Z-docosahexaenoyl)-glycero-3-phosphoserine; 1-(5Z,8Z,11Z,14Z-eicosatetraenoyl)-2-(4Z,7Z,10Z,13Z,16Z,19Z-docosahexaenoyl)-glycero-3-phosphoserine; 1-(9Z,12Z-octadecadienoyl)-2-(4Z,7Z,10Z,13Z,16Z,19Z-docosahexaenoyl)-glycero-3-phosphoserine; 1-octadecanoyl-2-tetradecanoyl-glycero-3-phosphoserine; 1-hexadecanoyl-2-dodecanoyl-glycero-3-phosphoserine; 1,2-dipentadecanoyl-sn-glycero-3-phosphoserine; 1-tetradecanoyl-2-hexadecanoyl-glycero-3-phosphoserine; 1-(2-methoxy-tetradecanyl)-sn-glycero-3-phosphoserine; 1-(2-methoxy-6Z-tetradecenyl)-sn-glycero-3-phosphoserine; 1-(2-methoxy-13-methyl-6Z-tetradecenyl)-sn-glycero-3-phosphoserine; 1-(2-methoxy-13-methyl-tetradecanyl)-sn-glycero-3-phosphoserine; 1-(2-methoxy-pentadecanyl)-sn-glycero-3-phosphoserine; 1-(2-methoxy-13-methyl-pentadecanyl)-sn-glycero-3-phosphoserine; 1-(2-methoxy-14-methyl-pentadecanyl)-sn-glycero-3-phosphoserine; 1-(2-methoxy-6Z-pentadecenyl)-sn-glycero-3-phosphoserine; 1-(2-methoxy-6Z-hexadecenyl)-sn-glycero-3-phosphoserine; 1-(2-methoxy-5Z-hexadecenyl)-sn-glycero-3-phosphoserine; 1-(2-methoxy-hexadecanyl)-sn-glycero-3-phosphoserine; 1-(2-methoxy-6Z-heptadecenyl)-sn-glycero-3-phosphoserine; 1-(2-methoxy-6Z-octadecenyl)-sn-glycero-3-phosphoserine; 1-(2-methoxy-octadecanyl)-sn-glycero-3-phosphoserine; 1-(2-methoxy-nonadecanyl)-sn-glycero-3-phosphoserine; 1-(2-methoxy-eicosanyl)-sn-glycero-3-phosphoserine; 1-(2-methoxy-heneicosanyl)-sn-glycero-3-phosphoserine; 1-(2-methoxy-docosanyl)-sn-glycero-3-phosphoserine; 1-(2-methoxy-tricosanyl)-sn-glycero-3-phosphoserine; 1-(2-methoxy-tetracosanyl)-sn-glycero-3-phosphoserine; 1-(2-methoxy-16Z-tricosenyl)-sn-glycero-3-phosphoserine; 1-(2-methoxy-17Z-tetracosenyl)-sn-glycero-3-phosphoserine; 1-(2-methoxy-18Z-pentacosenyl)-sn-glycero-3-phosphoserine; 1-(2-methoxy-19Z-hexacosenyl)-sn-glycero-3-phosphoserine; 1-(2-methoxy-20Z-heptacosenyl)-sn-glycero-3-phosphoserine; 1-(2-methoxy-21Z-octacosenyl)-sn-glycero-3-phosphoserine; 1-(2-methoxy-5Z,9Z-hexacosadienyl)-sn-glycero-3-phosphoserine; 1-(2-methoxy-5Z,19Z-hexacosadienyl)-sn-glycero-3-phosphoserine; 1-(2-methoxy-7Z,21Z-octacosadienyl)-sn-glycero-3-phosphoserine; 1-(7Z,10Z,13Z,16Z-docosatetraenoyl)-2-(4Z,7Z,10Z,13Z,16Z,19Z-docosahexaenoyl)-sn-glycero-3-phosphoserine; 1-acyl-sn-glycero-3-phosphoserine; 1-(Z)-alk-1-enyl-2-acyl-sn-glycero-3-phosphoserine; 1-(1Z-alkenyl)-sn-glycero-3-phosphoserine; 1-alkyl-2-acyl-sn-glycero-3-phosphoserine; and/or 1-alkyl-sn-glycero-3-phosphoserine.

In some embodiments, the glycerophospholipid may be a derivative of phosphatidyl glycerol. Phosphatidyl glycerol may be represented by formula (Ic):

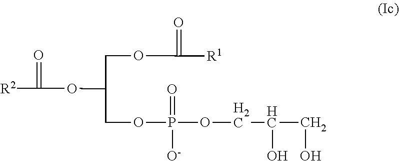

Non-limiting examples of derivatives of phosphatidyl glycerol are 1-hexadecanoyl-2-(9Z-octadecenoyl)-sn-glycero-3-phospho-(1′-rac-glycerol); 1-hexadecanoyl-2-(9Z-octadecenoyl)-sn-glycero-3-phospho-(1′-sn-glycerol); 2,3-ditetradecanoyl-sn-glycero-1-phospho-(2′-lyso-3′-tetradecanoyl-1′-sn-glycerol); 1-dodecanoyl-2-tridecanoyl-sn-glycero-3-phospho-(1′-rac-glycerol); 1-heneicosanoyl-2-(4Z,7Z,10Z,13Z,16Z,19Z-docosahexenoyl)-sn-glycero-3-phospho-(1′-rac-glycerol); 1,2-ditetradecanoyl-sn-glycero-3-phospho-(1′-sn-glycerol); 1-heptadecanoyl-2-(5Z,8Z,11Z,14Z-eicosatetraenoyl)-sn-glycero-3-phospho-(1′-rac-glycerol); 1-heptadecanoyl-2-(9Z-tetradecenoyl)-sn-glycero-3-phospho-(1′-rac-glycerol); Bis-[2-(5Z,8Z,11Z,14Z-eicosanoyl)-3-lyso-sn-glycero]-1-phosphate; 1-hexadecanoyl-2-(11Z-octadecenoyl)-sn-glycero-3-phospho-(1′-sn-glycerol); 1-tetradecanoyl-2-hexadecanoyl-sn-glycero-3-phospho-(1′-sn-glycerol); 1,2-dipentadecanoyl-sn-glycero-3-phospho-(1′-sn-glycerol); 1-hexadecanoyl-2-dodecanoyl-sn-glycero-3-phospho-(1′-sn-glycerol); 1-octadecanoyl-2-tetradecanoyl-sn-glycero-3-phospho-(1′-sn-glycerol); 1-(9Z-octadecenoyl)-sn-glycero-3-phospho-(1′-sn-glycerol); 1,2-dihexanoyl-sn-glycero-3-phospho-(1′-sn-glycerol); 1,2-dioctanoyl-sn-glycero-3-phospho-(1′-sn-glycerol); 1,2-didecanoyl-sn-glycero-3-phospho-(1′-sn-glycerol); 1,2-di-(9E-octadecenoyl)-sn-glycero-3-phospho-(1′-sn-glycerol); 1,2-diacyl-sn-glycero-3-phospho-(1′-sn-glycerol); 2,3-di-(9Z-octadecenoyl)-sn-glycero-1-phospho-(2′-lyso-3′-(9Z-octadecenoyl)-1′-sn-glycerol); 1,2-didodecanoyl-sn-glycero-3-phospho-(1′-sn-glycerol); 1-hexadecanoyl-2-(5Z,8Z,11Z,14Z-eicosatetraenoyl)-sn-glycero-3-phospho-(1′-sn-glycerol); 1-octadecanoyl-2-(9Z-octadecenoyl)-sn-glycero-3-phospho-(1′-sn-glycerol); 1,2-dioctadecanoyl-sn-glycero-3-phospho-(1′-sn-glycerol); 1-octadecanoyl-2-(5Z,8Z,11Z,14Z-eicosatetraenoyl)-sn-glycero-3-phospho-(1′-sn-glycerol); 1-octadecanoyl-2-(4Z,7Z,10Z,13Z,16Z,19Z-docosahexaenoyl)-sn-glycero-3-phospho-(1′-sn-glycerol); 1-tetradecanoyl-2-(8-[3]-ladderane-octanyl)-sn-glycero-3-phospho-(1′-sn-glycerol); 1-(6-[5]-ladderane-hexanyl)-2-(8-[3]-ladderane-octanyl)-sn-glycero-3-phospho-(1′-sn-glycerol); 1-tetradecanyl-2-(8-[3]-ladderane-octanyl)-sn-glycero-3-phospho-(1′-sn-glycerol); 1-(6-[5]-ladderane-hexanoyl)-2-(8-[3]-ladderane-octanyl)-sn-glycero-3-phospho-(1′-sn-glycerol); 1-(6-[3]-ladderane-hexanoyl)-2-(8-[3]-ladderane-octanyl)-sn-glycero-3-phospho-(1′-sn-glycerol); 1-(8-[5]-ladderane-octanyl)-2-(8-[3]-ladderane-octanyl)-sn-glycero-3-phospho-(1′-sn-glycerol); 1-(8-[3]-ladderane-octanyl)-2-(8-[3]-ladderane-octanyl)-sn-glycero-3-phospho-(1′-sn-glycerol); 1-(8-[5]-ladderane-octanoyl)-2-(8-[3]-ladderane-octanyl)-sn-glycero-3-phospho-(1′-sn-glycerol); 1-(8-[3]-ladderane-octanoyl)-2-(8-[3]-ladderane-octanyl)-sn-glycero-3-phospho-(1′-sn-glycerol); 1-(3-hydroxyphytanyl)-2-phytanyl-sn-glycero-3-phospho-(1′-sn-glycerol); 1-tridecanoyl-sn-glycero-3-phospho-(1′-sn-glycerol); 1-hexadecanoyl-sn-glycero-3-phospho-(1′-sn-glycerol); 1-octadecanoyl-sn-glycero-3-phospho-(1′-sn-glycerol); 1-(5Z,8Z,11Z,14Z-eicosatetraenoyl)-sn-glycero-3-phospho-(1′-sn-glycerol); 1-hexadecanoyl-2-(4Z,7Z,10Z,13Z,16Z,19Z-docosahexaenoyl)-glycero-3-phospho-(1′-sn-glycerol); 1-(9Z-octadecenoyl)-2-(4Z,7Z,10Z,13Z,16Z,19Z-docosahexaenoyl)-glycero-3-phospho-(1′-sn-glycerol); 1-(5Z,8Z,11Z,14Z-eicosatetraenoyl)-2-(4Z,7Z,10Z,13Z,16Z,19Z-docosahexaenoyl)-glycero-3-phospho-(1′-sn-glycerol); 1-(9Z,12Z-octadecadienoyl)-2-(4Z,7Z,10Z,13Z,16Z,19Z-docosahexaenoyl)-glycero-3-phospho-(1′-sn-glycerol); 1-dodecanoyl-glycero-3-phospho-(1′-sn-glycerol); 1-tetradecanoyl-glycero-3-phospho-(1′-sn-glycerol); 1-(9Z-hexadecenoyl)-glycero-3-phospho-(1′-sn-glycerol); 1-(9Z,12Z-octadecadienoyl)-glycero-3-phospho-(1′-sn-glycerol); 1-eicosanoyl-glycero-3-phospho-(1′-sn-glycerol); 1-(4Z,7Z,10Z,13Z,16Z,19Z-docosahexaenoyl)-glycero-3-phospho-(1′-sn-glycerol); 1-(7Z,10Z,13Z,16Z-docosatetraenoyl)-glycero-3-phospho-(1′-sn-glycerol); 1-dodecanoyl-2-(9Z-tetradecenoyl)-glycero-3-phospho-(1′-sn-glycerol); 1-dodecanoyl-2-pentadecanoyl-glycero-3-phospho-(1′-sn-glycerol); 1-dodecanoyl-2-(9Z-pentadecenoyl)-glycero-3-phospho-(1′-sn-glycerol); 1-dodecanoyl-2-(9Z-hexadecenoyl)-glycero-3-phospho-(1′-sn-glycerol); 1-dodecanoyl-2-heptadecanoyl-glycero-3-phospho-(1′-sn-glycerol); 1-dodecanoyl-2-(9Z-heptadecenoyl)-glycero-3-phospho-(1′-sn-glycerol); 1-dodecanoyl-2-(9Z,12Z-heptadecadienoyl)-glycero-3-phospho-(1′-sn-glycerol); 1-dodecanoyl-2-(9Z,12Z-octadecadienoyl)-glycero-3-phospho-(1′-sn-glycerol); 1-dodecanoyl-2-(6Z,9Z,12Z-octadecatrienoyl)-glycero-3-phospho-(1′-sn-glycerol); 1-dodecanoyl-2-(9Z,12Z,15Z-octadecatrienoyl)-glycero-3-phospho-(1′-sn-glycerol); 1-dodecanoyl-2-(6Z,9Z,12Z,15Z-octadecatetraenoyl)-glycero-3-phospho-(1′-sn-glycerol); 1-dodecanoyl-2-nonadecanoyl-glycero-3-phospho-(1′-sn-glycerol); 1-dodecanoyl-2-(9Z-nonadecenoyl)-glycero-3-phospho-(1′-sn-glycerol); 1-dodecanoyl-2-(11Z-eicosenoyl)-glycero-3-phospho-(1′-sn-glycerol); 1-dodecanoyl-2-(11Z,14Z-eicosadienoyl)-glycero-3-phospho-(1′-sn-glycerol); 1-dodecanoyl-2-(8Z,11Z,14Z-eicosatrienoyl)-glycero-3-phospho-(1′-sn-glycerol); 1-dodecanoyl-2-(5Z,8Z,11Z,14Z-eicosatetraenoyl)-glycero-3-phospho-(1′-sn-glycerol); 1-dodecanoyl-2-(5Z,8Z,11Z,14Z,17Z-eicosapentaenoyl)-glycero-3-phospho-(1′-sn-glycerol); 1-dodecanoyl-2-heneicosanoyl-glycero-3-phospho-(1′-sn-glycerol); 1-dodecanoyl-2-(11Z-docosenoyl)-glycero-3-phospho-(1′-sn-glycerol); 1-dodecanoyl-2-(13Z,16Z-docosadienoyl)-glycero-3-phospho-(1′-sn-glycerol); 1-dodecanoyl-2-(7Z,10Z,13Z,16Z-docosatetraenoyl)-glycero-3-phospho-(1′-sn-glycerol); 1-tridecanoyl-2-dodecanoyl-glycero-3-phospho-(1′-sn-glycerol); 1-tridecanoyl-2-tetradecanoyl-glycero-3-phospho-(1′-sn-glycerol); 1-tridecanoyl-2-(9Z-tetradecenoyl)-glycero-3-phospho-(1′-sn-glycerol); 1-tridecanoyl-2-(9Z-pentadecenoyl)-glycero-3-phospho-(1′-sn-glycerol); 1-tridecanoyl-2-hexadecanoyl-glycero-3-phospho-(1′-sn-glycerol); 1-tridecanoyl-2-(9Z-hexadecenoyl)-glycero-3-phospho-(1′-sn-glycerol); 1-tridecanoyl-2-(9Z-heptadecenoyl)-glycero-3-phospho-(1′-sn-glycerol); 1-tridecanoyl-2-(9Z,12Z-heptadecadienoyl)-glycero-3-phospho-(1′-sn-glycerol); 1-tridecanoyl-2-octadecanoyl-glycero-3-phospho-(1′-sn-glycerol); 1-tridecanoyl-2-(9Z-octadecenoyl)-glycero-3-phospho-(1′-sn-glycerol); 1-tridecanoyl-2-(9Z,12Z-octadecadienoyl)-glycero-3-phospho-(1′-sn-glycerol); 1-tridecanoyl-2-(6Z,9Z,12Z-octadecatrienoyl)-glycero-3-phospho-(1′-sn-glycerol); 1-tridecanoyl-2-(9Z,12Z,15Z-octadecatrienoyl)-glycero-3-phospho-(1′-sn-glycerol); 1-tridecanoyl-2-(6Z,9Z,12Z,15Z-octadecatetraenoyl)-glycero-3-phospho-(1′-sn-glycerol); 1-tridecanoyl-2-(9Z-nonadecenoyl)-glycero-3-phospho-(1′-sn-glycerol); 1-tridecanoyl-2-(11Z-eicosenoyl)-glycero-3-phospho-(1′-sn-glycerol); 1-tridecanoyl-2-(11Z,14Z-eicosadienoyl)-glycero-3-phospho-(1′-sn-glycerol); 1-tridecanoyl-2-(8Z,11Z,14Z-eicosatrienoyl)-glycero-3-phospho-(1′-sn-glycerol); 1-tridecanoyl-2-(5Z,8Z,11 Z,14Z-eicosatetraenoyl)-glycero-3-phospho-(1′-sn-glycerol); 1-tridecanoyl-2-(5Z,8Z,11Z,14Z,17Z-eicosapentaenoyl)-glycero-3-phospho-(1′-sn-glycerol); 1-tridecanoyl-2-docosanoyl-glycero-3-phospho-(1′-sn-glycerol); 1-tridecanoyl-2-(11Z-docosenoyl)-glycero-3-phospho-(1′-sn-glycerol); 1-tridecanoyl-2-(13Z,16Z-docosadienoyl)-glycero-3-phospho-(1′-sn-glycerol); 1-tridecanoyl-2-(7Z,10Z,13Z,16Z-docosatetraenoyl)-glycero-3-phospho-(1′-sn-glycerol); 1-tridecanoyl-2-(4Z,7Z,10Z,13Z,16Z,19Z-docosahexaenoyl)-glycero-3-phospho-(1′-sn-glycerol); 1-tetradecanoyl-2-tridecanoyl-glycero-3-phospho-(1′-sn-glycerol); 1-tetradecanoyl-2-(9Z-tetradecenoyl)-glycero-3-phospho-(1′-sn-glycerol); 1-tetradecanoyl-2-(9Z-pentadecenoyl)-glycero-3-phospho-(1′-sn-glycerol); 1-tetradecanoyl-2-heptadecanoyl-glycero-3-phospho-(1′-sn-glycerol); 1-tetradecanoyl-2-(9Z-heptadecenoyl)-glycero-3-phospho-(1′-sn-glycerol); 1-tetradecanoyl-2-(9Z,12Z-heptadecadienoyl)-glycero-3-phospho-(1′-sn-glycerol); 1-tetradecanoyl-2-(6Z,9Z,12Z-octadecatrienoyl)-glycero-3-phospho-(1′-sn-glycerol); 1-tetradecanoyl-2-nonadecanoyl-glycero-3-phospho-(1′-sn-glycerol); 1-tetradecanoyl-2-(9Z-nonadecenoyl)-glycero-3-phospho-(1′-sn-glycerol); 1-tetradecanoyl-2-(11Z-eicosenoyl)-glycero-3-phospho-(1′-sn-glycerol); 1-tetradecanoyl-2-(11Z,14Z-eicosadienoyl)-glycero-3-phospho-(1′-sn-glycerol); 1-tetradecanoyl-2-(8Z,11Z,14Z-eicosatrienoyl)-glycero-3-phospho-(1′-sn-glycerol); 1-tetradecanoyl-2-heneicosanoyl-glycero-3-phospho-(1′-sn-glycerol); 1-tetradecanoyl-2-(11Z-docosenoyl)-glycero-3-phospho-(1′-sn-glycerol); 1-tetradecanoyl-2-(13Z,16Z-docosadienoyl)-glycero-3-phospho-(1′-sn-glycerol); 1-tetradecanoyl-2-(7Z,10Z,13Z,16Z-docosatetraenoyl)-glycero-3-phospho-(1′-sn-glycerol); 1-(9Z-tetradecenoyl)-2-dodecanoyl-glycero-3-phospho-(1′-sn-glycerol); 1-(9Z-tetradecenoyl)-2-tridecanoyl-glycero-3-phospho-(1′-sn-glycerol); 1-(9Z-tetradecenoyl)-2-tetradecanoyl-glycero-3-phospho-(1′-sn-glycerol); 1-(9Z-tetradecenoyl)-2-pentadecanoyl-glycero-3-phospho-(1′-sn-glycerol); 1-(9Z-tetradecenoyl)-2-(9Z-pentadecenoyl)-glycero-3-phospho-(1′-sn-glycerol); 1-(9Z-tetradecenoyl)-2-hexadecanoyl-glycero-3-phospho-(1′-sn-glycerol); 1-(9Z-tetradecenoyl)-2-(9Z-hexadecenoyl)-glycero-3-phospho-(1′-sn-glycerol); 1-(9Z-tetradecenoyl)-2-heptadecanoyl-glycero-3-phospho-(1′-sn-glycerol); 1-(9Z-tetradecenoyl)-2-(9Z-heptadecenoyl)-glycero-3-phospho-(1′-sn-glycerol); 1-(9Z-tetradecenoyl)-2-(9Z,12Z-heptadecadienoyl)-glycero-3-phospho-(1′-sn-glycerol); 1-(9Z-tetradecenoyl)-2-octadecanoyl-glycero-3-phospho-(1′-sn-glycerol); 1-(9Z-tetradecenoyl)-2-(9Z-octadecenoyl)-glycero-3-phospho-(1′-sn-glycerol); 1-(9Z-tetradecenoyl)-2-(9Z,12Z-octadecadienoyl)-glycero-3-phospho-(1′-sn-glycerol); 1-(9Z-tetradecenoyl)-2-(6Z,9Z,12Z-octadecatrienoyl)-glycero-3-phospho-(1′-sn-glycerol); 1-(9Z-tetradecenoyl)-2-(9Z,12Z,15Z-octadecatrienoyl)-glycero-3-phospho-(1′-sn-glycerol); 1-(9Z-tetradecenoyl)-2-(6Z,9Z,12Z,15Z-octadecatetraenoyl)-glycero-3-phospho-(1′-sn-glycerol); 1-(9Z-tetradecenoyl)-2-nonadecanoyl-glycero-3-phospho-(1′-sn-glycerol); 1-(9Z-tetradecenoyl)-2-(9Z-nonadecenoyl)-glycero-3-phospho-(1′-sn-glycerol); 1-(9Z-tetradecenoyl)-2-eicosanoyl-glycero-3-phospho-(1′-sn-glycerol); 1-(9Z-tetradecenoyl)-2-(11Z-eicosenoyl)-glycero-3-phospho-(1′-sn-glycerol); 1-(9Z-tetradecenoyl)-2-(11Z,14Z-eicosadienoyl)-glycero-3-phospho-(1′-sn-glycerol); 1-(9Z-tetradecenoyl)-2-(8Z,11Z,14Z-eicosatrienoyl)-glycero-3-phospho-(1′-sn-glycerol); 1-(9Z-tetradecenoyl)-2-(5Z,8Z,11Z,14Z-eicosatetraenoyl)-glycero-3-phospho-(1′-sn-glycerol); 1-(9Z-tetradecenoyl)-2-(5Z,8Z,11Z,14Z,17Z-eicosapentaenoyl)-glycero-3-phospho-(1′-sn-glycerol); 1-(9Z-tetradecenoyl)-2-heneicosanoyl-glycero-3-phospho-(1′-sn-glycerol); 1-(9Z-tetradecenoyl)-2-docosanoyl-glycero-3-phospho-(1′-sn-glycerol); 1-(9Z-tetradecenoyl)-2-(11Z-docosenoyl)-glycero-3-phospho-(1′-sn-glycerol); 1-(9Z-tetradecenoyl)-2-(13Z,16Z-docosadienoyl)-glycero-3-phospho-(1′-sn-glycerol); 1-(9Z-tetradecenoyl)-2-(7Z,10Z,13Z,16Z-docosatetraenoyl)-glycero-3-phospho-(1′-sn-glycerol); 1-(9Z-tetradecenoyl)-2-(4Z,7Z,10Z,13Z,16Z,19Z-docosahexaenoyl)-glycero-3-phospho-(1′-sn-glycerol); 1-pentadecanoyl-2-dodecanoyl-glycero-3-phospho-(1′-sn-glycerol); 1-pentadecanoyl-2-tetradecanoyl-glycero-3-phospho-(1′-sn-glycerol); 1-pentadecanoyl-2-(9Z-tetradecenoyl)-glycero-3-phospho-(1′-sn-glycerol); 1-pentadecanoyl-2-(9Z-pentadecenoyl)-glycero-3-phospho-(1′-sn-glycerol); 1-pentadecanoyl-2-(9Z-heptadecenoyl)-glycero-3-phospho-(1′-sn-glycerol); 1-pentadecanoyl-2-(9Z,12Z-heptadecadienoyl)-glycero-3-phospho-(1′-sn-glycerol); 1-pentadecanoyl-2-(9Z-octadecenoyl)-glycero-3-phospho-(1′-sn-glycerol); 1-pentadecanoyl-2-(6Z,9Z,12Z-octadecatrienoyl)-glycero-3-phospho-(1′-sn-glycerol); 1-pentadecanoyl-2-(9Z,12Z,15Z-octadecatrienoyl)-glycero-3-phospho-(1′-sn-glycerol); 1-pentadecanoyl-2-(6Z,9Z,12Z,15Z-octadecatetraenoyl)-glycero-3-phospho-(1′-sn-glycerol); 1-pentadecanoyl-2-(9Z-nonadecenoyl)-glycero-3-phospho-(1′-sn-glycerol); 1-pentadecanoyl-2-eicosanoyl-glycero-3-phospho-(1′-sn-glycerol); 1-pentadecanoyl-2-(11Z-eicosenoyl)-glycero-3-phospho-(1′-sn-glycerol); 1-pentadecanoyl-2-(11Z,14Z-eicosadienoyl)-glycero-3-phospho-(1′-sn-glycerol); 1-pentadecanoyl-2-(8Z,11Z,14Z-eicosatrienoyl)-glycero-3-phospho-(1′-sn-glycerol); 1-pentadecanoyl-2-(5Z,8Z,11Z,14Z-eicosatetraenoyl)-glycero-3-phospho-(1′-sn-glycerol); 1-pentadecanoyl-2-(5Z,8Z,11Z,14Z,17Z-eicosapentaenoyl)-glycero-3-phospho-(1′-sn-glycerol); 1-pentadecanoyl-2-docosanoyl-glycero-3-phospho-(1′-sn-glycerol); 1-pentadecanoyl-2-(11Z-docosenoyl)-glycero-3-phospho-(1′-sn-glycerol); 1-pentadecanoyl-2-(13Z,16Z-docosadienoyl)-glycero-3-phospho-(1′-sn-glycerol); 1-pentadecanoyl-2-(7Z,10Z,13Z,16Z-docosatetraenoyl)-glycero-3-phospho-(1′-sn-glycerol); 1-pentadecanoyl-2-(4Z,7Z,10Z,13Z,16Z,19Z-docosahexaenoyl)-glycero-3-phospho-(1′-sn-glycerol); 1-(9Z-pentadecenoyl)-glycero-3-phospho-(1′-sn-glycerol); 1-(9Z-pentadecenoyl)-2-dodecanoyl-glycero-3-phospho-(1′-sn-glycerol); 1-(9Z-pentadecenoyl)-2-tridecanoyl-glycero-3-phospho-(1′-sn-glycerol); 1-(9Z-pentadecenoyl)-2-tetradecanoyl-glycero-3-phospho-(1′-sn-glycerol); 1-(9Z-pentadecenoyl)-2-(9Z-tetradecenoyl)-glycero-3-phospho-(1′-sn-glycerol); 1-(9Z-pentadecenoyl)-2-pentadecanoyl-glycero-3-phospho-(1′-sn-glycerol); 1,2-di-(9Z-pentadecenoyl)-sn-glycero-3-phospho-(1′-sn-glycerol); 1-(9Z-pentadecenoyl)-2-hexadecanoyl-glycero-3-phospho-(1′-sn-glycerol); 1-(9Z-pentadecenoyl)-2-(9Z-hexadecenoyl)-glycero-3-phospho-(1′-sn-glycerol); 1-(9Z-pentadecenoyl)-2-heptadecanoyl-glycero-3-phospho-(1′-sn-glycerol); 1-(9Z-pentadecenoyl)-2-(9Z-heptadecenoyl)-glycero-3-phospho-(1′-sn-glycerol); 1-(9Z-pentadecenoyl)-2-(9Z,12Z-heptadecadienoyl)-glycero-3-phospho-(1′-sn-glycerol); 1-(9Z-pentadecenoyl)-2-octadecanoyl-glycero-3-phospho-(1′-sn-glycerol); 1-(9Z-pentadecenoyl)-2-(9Z-octadecenoyl)-glycero-3-phospho-(1′-sn-glycerol); 1-(9Z-pentadecenoyl)-2-(9Z,12Z-octadecadienoyl)-glycero-3-phospho-(1′-sn-glycerol); 1-(9Z-pentadecenoyl)-2-(6Z,9Z,12Z-octadecatrienoyl)-glycero-3-phospho-(1′-sn-glycerol); 1-(9Z-pentadecenoyl)-2-(9Z,12Z,15Z-octadecatrienoyl)-glycero-3-phospho-(1′-sn-glycerol); 1-(9Z-pentadecenoyl)-2-(6Z,9Z,12Z,15Z-octadecatetraenoyl)-glycero-3-phospho-(1′-sn-glycerol); 1-(9Z-pentadecenoyl)-2-nonadecanoyl-glycero-3-phospho-(1′-sn-glycerol); 1-(9Z-pentadecenoyl)-2-(9Z-nonadecenoyl)-glycero-3-phospho-(1′-sn-glycerol); 1-(9Z-pentadecenoyl)-2-eicosanoyl-glycero-3-phospho-(1′-sn-glycerol); 1-(9Z-pentadecenoyl)-2-(11Z-eicosenoyl)-glycero-3-phospho-(1′-sn-glycerol); 1-(9Z-pentadecenoyl)-2-(11Z,14Z-eicosadienoyl)-glycero-3-phospho-(1′-sn-glycerol); 1-(9Z-pentadecenoyl)-2-(8Z,11Z,14Z-eicosatrienoyl)-glycero-3-phospho-(1′-sn-glycerol); 1-(9Z-pentadecenoyl)-2-(5Z,8Z,11Z,14Z-eicosatetraenoyl)-glycero-3-phospho-(1′-sn-glycerol); 1-(9Z-pentadecenoyl)-2-(5Z,8Z,11Z,14Z,17Z-eicosapentaenoyl)-glycero-3-phospho-(1′-sn-glycerol); 1-(9Z-pentadecenoyl)-2-heneicosanoyl-glycero-3-phospho-(1′-sn-glycerol); 1-(9Z-pentadecenoyl)-2-docosanoyl-glycero-3-phospho-(1′-sn-glycerol); 1-(9Z-pentadecenoyl)-2-(11Z-docosenoyl)-glycero-3-phospho-(1′-sn-glycerol); 1-(9Z-pentadecenoyl)-2-(13Z,16Z-docosadienoyl)-glycero-3-phospho-(1′-sn-glycerol); 1-(9Z-pentadecenoyl)-2-(7Z,10Z,13Z,16Z-docosatetraenoyl)-glycero-3-phospho-(1′-sn-glycerol); 1-(9Z-pentadecenoyl)-2-(4Z,7Z,10Z,13Z,16Z,19Z-docosahexaenoyl)-glycero-3-phospho-(1′-sn-glycerol); 1-hexadecanoyl-2-tridecanoyl-glycero-3-phospho-(1′-sn-glycerol); 1-hexadecanoyl-2-(9Z-tetradecenoyl)-glycero-3-phospho-(1′-sn-glycerol); 1-hexadecanoyl-2-(9Z-pentadecenoyl)-glycero-3-phospho-(1′-sn-glycerol); 1-hexadecanoyl-2-(9Z,12Z-heptadecadienoyl)-glycero-3-phospho-(1′-sn-glycerol); 1-hexadecanoyl-2-(9Z-nonadecenoyl)-glycero-3-phospho-(1′-sn-glycerol); 1-hexadecanoyl-2-(11Z,14Z-eicosadienoyl)-glycero-3-phospho-(1′-sn-glycerol); 1-hexadecanoyl-2-heneicosanoyl-glycero-3-phospho-(1′-sn-glycerol); 1-hexadecanoyl-2-(11Z-docosenoyl)-glycero-3-phospho-(1′-sn-glycerol); 1-hexadecanoyl-2-(13Z,16Z-docosadienoyl)-glycero-3-phospho-(1′-sn-glycerol); 1-(9Z-hexadecenoyl)-2-dodecanoyl-glycero-3-phospho-(1′-sn-glycerol); 1-(9Z-hexadecenoyl)-2-tridecanoyl-glycero-3-phospho-(1′-sn-glycerol); 1-(9Z-hexadecenoyl)-2-tetradecanoyl-glycero-3-phospho-(1′-sn-glycerol); 1-(9Z-hexadecenoyl)-2-(9Z-tetradecenoyl)-glycero-3-phospho-(1′-sn-glycerol); 1-(9Z-hexadecenoyl)-2-pentadecanoyl-glycero-3-phospho-(1′-sn-glycerol); 1-(9Z-hexadecenoyl)-2-(9Z-pentadecenoyl)-glycero-3-phospho-(1′-sn-glycerol); 1-(9Z-hexadecenoyl)-2-hexadecanoyl-glycero-3-phospho-(1′-sn-glycerol); 1-(9Z-hexadecenoyl)-2-heptadecanoyl-glycero-3-phospho-(1′-sn-glycerol); 1-(9Z-hexadecenoyl)-2-(9Z-heptadecenoyl)-glycero-3-phospho-(1′-sn-glycerol); 1-(9Z-hexadecenoyl)-2-(9Z,12Z-heptadecadienoyl)-glycero-3-phospho-(1′-sn-glycerol); 1-(9Z-hexadecenoyl)-2-octadecanoyl-glycero-3-phospho-(1′-sn-glycerol); 1-(9Z-hexadecenoyl)-2-(6Z,9Z,12Z-octadecatrienoyl)-glycero-3-phospho-(1′-sn-glycerol); 1-(9Z-hexadecenoyl)-2-(9Z,12Z,15Z-octadecatrienoyl)-glycero-3-phospho-(1′-sn-glycerol); 1-(9Z-hexadecenoyl)-2-nonadecanoyl-glycero-3-phospho-(1′-sn-glycerol); 1-(9Z-hexadecenoyl)-2-(9Z-nonadecenoyl)-glycero-3-phospho-(1′-sn-glycerol); 1-(9Z-hexadecenoyl)-2-eicosanoyl-glycero-3-phospho-(1′-sn-glycerol); 1-(9Z-hexadecenoyl)-2-(11Z-eicosenoyl)-glycero-3-phospho-(1′-sn-glycerol); 1-(9Z-hexadecenoyl)-2-(11Z,14Z-eicosadienoyl)-glycero-3-phospho-(1′-sn-glycerol); 1-(9Z-hexadecenoyl)-2-(8Z,11Z,14Z-eicosatrienoyl)-glycero-3-phospho-(1′-sn-glycerol); 1-(9Z-hexadecenoyl)-2-(5Z,8Z,11Z,14Z,17Z-eicosapentaenoyl)-glycero-3-phospho-(1′-sn-glycerol); 1-(9Z-hexadecenoyl)-2-heneicosanoyl-glycero-3-phospho-(1′-sn-glycerol); 1-(9Z-hexadecenoyl)-2-docosanoyl-glycero-3-phospho-(1′-sn-glycerol); 1-(9Z-hexadecenoyl)-2-(11Z-docosenoyl)-glycero-3-phospho-(1′-sn-glycerol); 1-(9Z-hexadecenoyl)-2-(13Z,16Z-docosadienoyl)-glycero-3-phospho-(1′-sn-glycerol); 1-(9Z-hexadecenoyl)-2-(7Z,10Z,13Z,16Z-docosatetraenoyl)-glycero-3-phospho-(1′-sn-glycerol); 1-heptadecanoyl-2-dodecanoyl-glycero-3-phospho-(1′-sn-glycerol); 1-heptadecanoyl-2-tetradecanoyl-glycero-3-phospho-(1′-sn-glycerol); 1-heptadecanoyl-2-(9Z-pentadecenoyl)-glycero-3-phospho-(1′-sn-glycerol); 1-heptadecanoyl-2-hexadecanoyl-glycero-3-phospho-(1′-sn-glycerol); 1-heptadecanoyl-2-(9Z-hexadecenoyl)-glycero-3-phospho-(1′-sn-glycerol); 1-heptadecanoyl-2-(9Z-heptadecenoyl)-glycero-3-phospho-(1′-sn-glycerol); 1-heptadecanoyl-2-(9Z,12Z-heptadecadienoyl)-glycero-3-phospho-(1′-sn-glycerol); 1-heptadecanoyl-2-(9Z,12Z-octadecadienoyl)-glycero-3-phospho-(1′-sn-glycerol); 1-heptadecanoyl-2-(6Z,9Z,12Z-octadecatrienoyl)-glycero-3-phospho-(1′-sn-glycerol); 1-heptadecanoyl-2-(9Z,12Z,15Z-octadecatrienoyl)-glycero-3-phospho-(1′-sn-glycerol); 1-heptadecanoyl-2-(6Z,9Z,12Z,15Z-octadecatetraenoyl)-glycero-3-phospho-(1′-sn-glycerol); 1-heptadecanoyl-2-nonadecanoyl-glycero-3-phospho-(1′-sn-glycerol); 1-heptadecanoyl-2-(9Z-nonadecenoyl)-glycero-3-phospho-(1′-sn-glycerol); 1-heptadecanoyl-2-(11Z-eicosenoyl)-glycero-3-phospho-(1′-sn-glycerol); 1-heptadecanoyl-2-(11Z,14Z-eicosadienoyl)-glycero-3-phospho-(1′-sn-glycerol); 1-heptadecanoyl-2-(8Z,11Z,14Z-eicosatrienoyl)-glycero-3-phospho-(1′-sn-glycerol); 1-heptadecanoyl-2-(5Z,8Z,11Z,14Z,17Z-eicosapentaenoyl)-glycero-3-phospho-(1′-sn-glycerol); 1-heptadecanoyl-2-heneicosanoyl-glycero-3-phospho-(1′-sn-glycerol); 1-heptadecanoyl-2-docosanoyl-glycero-3-phospho-(1′-sn-glycerol); 1-heptadecanoyl-2-(11Z-docosenoyl)-glycero-3-phospho-(1′-sn-glycerol); 1-heptadecanoyl-2-(13Z,16Z-docosadienoyl)-glycero-3-phospho-(1′-sn-glycerol); 1-heptadecanoyl-2-(7Z,10Z,13Z,16Z-docosatetraenoyl)-glycero-3-phospho-(1′-sn-glycerol); 1-(9Z-heptadecenoyl)-2-dodecanoyl-glycero-3-phospho-(1′-sn-glycerol); 1-(9Z-heptadecenoyl)-2-tridecanoyl-glycero-3-phospho-(1′-sn-glycerol); 1-(9Z-heptadecenoyl)-2-tetradecanoyl-glycero-3-phospho-(1′-sn-glycerol); 1-(9Z-heptadecenoyl)-2-(9Z-tetradecenoyl)-glycero-3-phospho-(1′-sn-glycerol); 1-(9Z-heptadecenoyl)-2-pentadecanoyl-glycero-3-phospho-(1′-sn-glycerol); 1-(9Z-heptadecenoyl)-2-(9Z-pentadecenoyl)-glycero-3-phospho-(1′-sn-glycerol); 1-(9Z-heptadecenoyl)-2-hexadecanoyl-glycero-3-phospho-(1′-sn-glycerol); 1-(9Z-heptadecenoyl)-2-(9Z-hexadecenoyl)-glycero-3-phospho-(1′-sn-glycerol); 1-(9Z-heptadecenoyl)-2-heptadecanoyl-glycero-3-phospho-(1′-sn-glycerol); 1-(9Z-heptadecenoyl)-2-(9Z,12Z-heptadecadienoyl)-glycero-3-phospho-(1′-sn-glycerol); 1-(9Z-heptadecenoyl)-2-octadecanoyl-glycero-3-phospho-(1′-sn-glycerol); 1-(9Z-heptadecenoyl)-2-(9Z-octadecenoyl)-glycero-3-phospho-(1′-sn-glycerol); 1-(9Z-heptadecenoyl)-2-(9Z,12Z-octadecadienoyl)-glycero-3-phospho-(1′-sn-glycerol); 1-(9Z-heptadecenoyl)-2-(6Z,9Z,12Z-octadecatrienoyl)-glycero-3-phospho-(1′-sn-glycerol); 1-(9Z-heptadecenoyl)-2-(9Z,12Z,15Z-octadecatrienoyl)-glycero-3-phospho-(1′-sn-glycerol); 1-(9Z-heptadecenoyl)-2-(6Z,9Z,12Z,15Z-octadecatetraenoyl)-glycero-3-phospho-(1′-sn-glycerol); 1-(9Z-heptadecenoyl)-2-nonadecanoyl-glycero-3-phospho-(1′-sn-glycerol); 1-(9Z-heptadecenoyl)-2-(9Z-nonadecenoyl)-glycero-3-phospho-(1′-sn-glycerol); 1-(9Z-heptadecenoyl)-2-eicosanoyl-glycero-3-phospho-(1′-sn-glycerol); 1-(9Z-heptadecenoyl)-2-(11Z-eicosenoyl)-glycero-3-phospho-(1′-sn-glycerol); 1-(9Z-heptadecenoyl)-2-(11Z,14Z-eicosadienoyl)-glycero-3-phospho-(1′-sn-glycerol); 1-(9Z-heptadecenoyl)-2-(8Z,11Z,14Z-eicosatrienoyl)-glycero-3-phospho-(1′-sn-glycerol); 1-(9Z-heptadecenoyl)-2-(5Z,8Z,11Z,14Z-eicosatetraenoyl)-glycero-3-phospho-(1′-sn-glycerol); 1-(9Z-heptadecenoyl)-2-(5Z,8Z,11Z,14Z,17Z-eicosapentaenoyl)-glycero-3-phospho-(1′-sn-glycerol); 1-(9Z-heptadecenoyl)-2-heneicosanoyl-glycero-3-phospho-(1′-sn-glycerol); 1-(9Z-heptadecenoyl)-2-docosanoyl-glycero-3-phospho-(1′-sn-glycerol); 1-(9Z-heptadecenoyl)-2-(11Z-docosenoyl)-glycero-3-phospho-(1′-sn-glycerol); 1-(9Z-heptadecenoyl)-2-(13Z,16Z-docosadienoyl)-glycero-3-phospho-(1′-sn-glycerol); 1-(9Z-heptadecenoyl)-2-(7Z,10Z,13Z,16Z-docosatetraenoyl)-glycero-3-phospho-(1′-sn-glycerol); 1-(9Z-heptadecenoyl)-2-(4Z,7Z,10Z,13Z,16Z,19Z-docosahexaenoyl)-glycero-3-phospho-(1′-sn-glycerol); 1-(9Z,12Z-heptadecadienoyl)-glycero-3-phospho-(1′-sn-glycerol); 1-(9Z,12Z-heptadecadienoyl)-2-dodecanoyl-glycero-3-phospho-(1′-sn-glycerol); 1-(9Z,12Z-heptadecadienoyl)-2-tridecanoyl-glycero-3-phospho-(1′-sn-glycerol); 1-(9Z,12Z-heptadecadienoyl)-2-tetradecanoyl-glycero-3-phospho-(1′-sn-glycerol); 1-(9Z,12Z-heptadecadienoyl)-2-(9Z-tetradecenoyl)-glycero-3-phospho-(1′-sn-glycerol); 1-(9Z,12Z-heptadecadienoyl)-2-pentadecanoyl-glycero-3-phospho-(1′-sn-glycerol); 1-(9Z,12Z-heptadecadienoyl)-2-(9Z-pentadecenoyl)-glycero-3-phospho-(1′-sn-glycerol); 1-(9Z,12Z-heptadecadienoyl)-2-hexadecanoyl-glycero-3-phospho-(1′-sn-glycerol); 1-(9Z,12Z-heptadecadienoyl)-2-(9Z-hexadecenoyl)-glycero-3-phospho-(1′-sn-glycerol); 1-(9Z,12Z-heptadecadienoyl)-2-heptadecanoyl-glycero-3-phospho-(1′-sn-glycerol); 1-(9Z,12Z-heptadecadienoyl)-2-(9Z-heptadecenoyl)-glycero-3-phospho-(1′-sn-glycerol); 1-(9Z,12Z-heptadecadienoyl)-2-octadecanoyl-glycero-3-phospho-(1′-sn-glycerol); 1-(9Z,12Z-heptadecadienoyl)-2-(9Z-octadecenoyl)-glycero-3-phospho-(1′-sn-glycerol); 1-(9Z,12Z-heptadecadienoyl)-2-(9Z,12Z-octadecadienoyl)-glycero-3-phospho-(1′-sn-glycerol); 1-(9Z,12Z-heptadecadienoyl)-2-(6Z,9Z,12Z-octadecatrienoyl)-glycero-3-phospho-(1′-sn-glycerol); 1-(9Z,12Z-heptadecadienoyl)-2-(9Z,12Z,15Z-octadecatrienoyl)-glycero-3-phospho-(1′-sn-glycerol); 1-(9Z,12Z-heptadecadienoyl)-2-(6Z,9Z,12Z,15Z-octadecatetraenoyl)-glycero-3-phospho-(1′-sn-glycerol); 1-(9Z,12Z-heptadecadienoyl)-2-nonadecanoyl-glycero-3-phospho-(1′-sn-glycerol); 1-(9Z,12Z-heptadecadienoyl)-2-(9Z-nonadecenoyl)-glycero-3-phospho-(1′-sn-glycerol); 1-(9Z,12Z-heptadecadienoyl)-2-eicosanoyl-glycero-3-phospho-(1′-sn-glycerol); 1-(9Z,12Z-heptadecadienoyl)-2-(11Z-eicosenoyl)-glycero-3-phospho-(1′-sn-glycerol); 1-(9Z,12Z-heptadecadienoyl)-2-(11Z,14Z-eicosadienoyl)-glycero-3-phospho-(1′-sn-glycerol); 1-(9Z,12Z-heptadecadienoyl)-2-(8Z,11Z,14Z-eicosatrienoyl)-glycero-3-phospho-(1′-sn-glycerol); 1-(9Z,12Z-heptadecadienoyl)-2-(5Z,8Z,11Z,14Z-eicosatetraenoyl)-glycero-3-phospho-(1′-sn-glycerol); 1-(9Z,12Z-heptadecadienoyl)-2-(5Z,8Z,11Z,14Z,17Z-eicosapentaenoyl)-glycero-3-phospho-(1′-sn-glycerol); 1-(9Z,12Z-heptadecadienoyl)-2-heneicosanoyl-glycero-3-phospho-(1′-sn-glycerol); 1-(9Z,12Z-heptadecadienoyl)-2-docosanoyl-glycero-3-phospho-(1′-sn-glycerol); 1-(9Z,12Z-heptadecadienoyl)-2-(11Z-docosenoyl)-glycero-3-phospho-(1′-sn-glycerol); 1-(9Z,12Z-heptadecadienoyl)-2-(13Z,16Z-docosadienoyl)-glycero-3-phospho-(1′-sn-glycerol); 1-(9Z,12Z-heptadecadienoyl)-2-(7Z,10Z,13Z,16Z-docosatetraenoyl)-glycero-3-phospho-(1′-sn-glycerol); 1-(9Z,12Z-heptadecadienoyl)-2-(4Z,7Z,10Z,13Z,16Z,19Z-docosahexaenoyl)-glycero-3-phospho-(1′-sn-glycerol); 1-octadecanoyl-2-tridecanoyl-glycero-3-phospho-(1′-sn-glycerol); 1-octadecanoyl-2-(9Z-tetradecenoyl)-glycero-3-phospho-(1′-sn-glycerol); 1-octadecanoyl-2-pentadecanoyl-glycero-3-phospho-(1′-sn-glycerol); 1-octadecanoyl-2-(9Z-pentadecenoyl)-glycero-3-phospho-(1′-sn-glycerol); 1-octadecanoyl-2-heptadecanoyl-glycero-3-phospho-(1′-sn-glycerol); 1-octadecanoyl-2-(9Z-heptadecenoyl)-glycero-3-phospho-(1′-sn-glycerol); 1-octadecanoyl-2-(9Z,12Z-heptadecadienoyl)-glycero-3-phospho-(1′-sn-glycerol); 1-octadecanoyl-2-(6Z,9Z,12Z-octadecatrienoyl)-glycero-3-phospho-(1′-sn-glycerol); 1-octadecanoyl-2-(9Z,12Z,15Z-octadecatrienoyl)-glycero-3-phospho-(1′-sn-glycerol); 1-octadecanoyl-2-nonadecanoyl-glycero-3-phospho-(1′-sn-glycerol); 1-octadecanoyl-2-(9Z-nonadecenoyl)-glycero-3-phospho-(1′-sn-glycerol); 1-octadecanoyl-2-heneicosanoyl-glycero-3-phospho-(1′-sn-glycerol); 1-octadecanoyl-2-(11Z-docosenoyl)-glycero-3-phospho-(1′-sn-glycerol); 1-octadecanoyl-2-(13Z,16Z-docosadienoyl)-glycero-3-phospho-(1′-sn-glycerol); 1-(9Z-octadecenoyl)-2-dodecanoyl-glycero-3-phospho-(1′-sn-glycerol); 1-(9Z-octadecenoyl)-2-tridecanoyl-glycero-3-phospho-(1′-sn-glycerol); 1-(9Z-octadecenoyl)-2-(9Z-tetradecenoyl)-glycero-3-phospho-(1′-sn-glycerol); 1-(9Z-octadecenoyl)-2-pentadecanoyl-glycero-3-phospho-(1′-sn-glycerol); 1-(9Z-octadecenoyl)-2-(9Z-pentadecenoyl)-glycero-3-phospho-(1′-sn-glycerol); 1-(9Z-octadecenoyl)-2-heptadecanoyl-glycero-3-phospho-(1′-sn-glycerol); 1-(9Z-octadecenoyl)-2-(9Z-heptadecenoyl)-glycero-3-phospho-(1′-sn-glycerol); 1-(9Z-octadecenoyl)-2-(9Z,12Z-heptadecadienoyl)-glycero-3-phospho-(1′-sn-glycerol); 1-(9Z-octadecenoyl)-2-(6Z,9Z,12Z-octadecatrienoyl)-glycero-3-phospho-(1′-sn-glycerol); 1-(9Z-octadecenoyl)-2-(6Z,9Z,12Z,15Z-octadecatetraenoyl)-glycero-3-phospho-(1′-sn-glycerol); 1-(9Z-octadecenoyl)-2-nonadecanoyl-glycero-3-phospho-(1′-sn-glycerol); 1-(9Z-octadecenoyl)-2-(9Z-nonadecenoyl)-glycero-3-phospho-(1′-sn-glycerol); 1-(9Z-octadecenoyl)-2-(11Z,14Z-eicosadienoyl)-glycero-3-phospho-(1′-sn-glycerol); 1-(9Z-octadecenoyl)-2-(8Z,11Z,14Z-eicosatrienoyl)-glycero-3-phospho-(1′-sn-glycerol); 1-(9Z-octadecenoyl)-2-heneicosanoyl-glycero-3-phospho-(1′-sn-glycerol); 1-(9Z-octadecenoyl)-2-(11Z-docosenoyl)-glycero-3-phospho-(1′-sn-glycerol); 1-(9Z-octadecenoyl)-2-(13Z,16Z-docosadienoyl)-glycero-3-phospho-(1′-sn-glycerol); 1-(9Z-octadecenoyl)-2-(7Z,10Z,13Z,16Z-docosatetraenoyl)-glycero-3-phospho-(1′-sn-glycerol); 1-(9Z,12Z-octadecadienoyl)-2-dodecanoyl-glycero-3-phospho-(1′-sn-glycerol); 1-(9Z,12Z-octadecadienoyl)-2-tridecanoyl-glycero-3-phospho-(1′-sn-glycerol); 1-(9Z,12Z-octadecadienoyl)-2-tetradecanoyl-glycero-3-phospho-(1′-sn-glycerol); 1-(9Z,12Z-octadecadienoyl)-2-(9Z-tetradecenoyl)-glycero-3-phospho-(1′-sn-glycerol); 1-(9Z,12Z-octadecadienoyl)-2-pentadecanoyl-glycero-3-phospho-(1′-sn-glycerol); 1-(9Z,12Z-octadecadienoyl)-2-(9Z-pentadecenoyl)-glycero-3-phospho-(1′-sn-glycerol); 1-(9Z,12Z-octadecadienoyl)-2-(9Z-hexadecenoyl)-glycero-3-phospho-(1′-sn-glycerol); 1-(9Z,12Z-octadecadienoyl)-2-heptadecanoyl-glycero-3-phospho-(1′-sn-glycerol); 1-(9Z,12Z-octadecadienoyl)-2-(9Z-heptadecenoyl)-glycero-3-phospho-(1′-sn-glycerol); 1-(9Z,12Z-octadecadienoyl)-2-(9Z,12Z-heptadecadienoyl)-glycero-3-phospho-(1′-sn-glycerol); 1-(9Z,12Z-octadecadienoyl)-2-(9Z-octadecenoyl)-glycero-3-phospho-(1′-sn-glycerol); 1-(9Z,12Z-octadecadienoyl)-2-(6Z,9Z,12Z-octadecatrienoyl)-glycero-3-phospho-(1′-sn-glycerol); 1-(9Z,12Z-octadecadienoyl)-2-(9Z,12Z,15Z-octadecatrienoyl)-glycero-3-phospho-(1′-sn-glycerol); 1-(9Z,12Z-octadecadienoyl)-2-(6Z,9Z,12Z,15Z-octadecatetraenoyl)-glycero-3-phospho-(1′-sn-glycerol); 1-(9Z,12Z-octadecadienoyl)-2-nonadecanoyl-glycero-3-phospho-(1′-sn-glycerol); 1-(9Z,12Z-octadecadienoyl)-2-(9Z-nonadecenoyl)-glycero-3-phospho-(1′-sn-glycerol); 1-(9Z,12Z-octadecadienoyl)-2-eicosanoyl-glycero-3-phospho-(1′-sn-glycerol); 1-(9Z,12Z-octadecadienoyl)-2-(11Z-eicosenoyl)-glycero-3-phospho-(1′-sn-glycerol); 1-(9Z,12Z-octadecadienoyl)-2-(11Z,14Z-eicosadienoyl)-glycero-3-phospho-(1′-sn-glycerol); 1-(9Z,12Z-octadecadienoyl)-2-(8Z,11Z,14Z-eicosatrienoyl)-glycero-3-phospho-(1′-sn-glycerol); 1-(9Z,12Z-octadecadienoyl)-2-(5Z,8Z,11Z,14Z,17Z-eicosapentaenoyl)-glycero-3-phospho-(1′-sn-glycerol); 1-(9Z,12Z-octadecadienoyl)-2-heneicosanoyl-glycero-3-phospho-(1′-sn-glycerol); 1-(9Z,12Z-octadecadienoyl)-2-docosanoyl-glycero-3-phospho-(1′-sn-glycerol); 1-(9Z,12Z-octadecadienoyl)-2-(11Z-docosenoyl)-glycero-3-phospho-(1′-sn-glycerol); 1-(9Z,12Z-octadecadienoyl)-2-(13Z,16Z-docosadienoyl)-glycero-3-phospho-(1′-sn-glycerol); 1-(9Z,12Z-octadecadienoyl)-2-(7Z,10Z,13Z,16Z-docosatetraenoyl)-glycero-3-phospho-(1′-sn-glycerol); 1-(6Z,9Z,12Z-octadecatrienoyl)-glycero-3-phospho-(1′-sn-glycerol); 1-(6Z,9Z,12Z-octadecatrienoyl)-2-dodecanoyl-glycero-3-phospho-(1′-sn-glycerol); 1-(6Z,9Z,12Z-octadecatrienoyl)-2-tridecanoyl-glycero-3-phospho-(1′-sn-glycerol); 1-(6Z,9Z,12Z-octadecatrienoyl)-2-tetradecanoyl-glycero-3-phospho-(1′-sn-glycerol); 1-(6Z,9Z,12Z-octadecatrienoyl)-2-(9Z-tetradecenoyl)-glycero-3-phospho-(1′-sn-glycerol); 1-(6Z,9Z,12Z-octadecatrienoyl)-2-pentadecanoyl-glycero-3-phospho-(1′-sn-glycerol); 1-(6Z,9Z,12Z-octadecatrienoyl)-2-(9Z-pentadecenoyl)-glycero-3-phospho-(1′-sn-glycerol); 1-(6Z,9Z,12Z-octadecatrienoyl)-2-hexadecanoyl-glycero-3-phospho-(1′-sn-glycerol); 1-(6Z,9Z,12Z-octadecatrienoyl)-2-(9Z-hexadecenoyl)-glycero-3-phospho-(1′-sn-glycerol); 1-(6Z,9Z,12Z-octadecatrienoyl)-2-heptadecanoyl-glycero-3-phospho-(1′-sn-glycerol); 1-(6Z,9Z,12Z-octadecatrienoyl)-2-(9Z-heptadecenoyl)-glycero-3-phospho-(1′-sn-glycerol); 1-(6Z,9Z,12Z-octadecatrienoyl)-2-(9Z,12Z-heptadecadienoyl)-glycero-3-phospho-(1′-sn-glycerol); 1-(6Z,9Z,12Z-octadecatrienoyl)-2-octadecanoyl-glycero-3-phospho-(1′-sn-glycerol); 1-(6Z,9Z,12Z-octadecatrienoyl)-2-(9Z-octadecenoyl)-glycero-3-phospho-(1′-sn-glycerol); 1-(6Z,9Z,12Z-octadecatrienoyl)-2-(9Z,12Z-octadecadienoyl)-glycero-3-phospho-(1′-sn-glycerol); 1,2-di-(6Z,9Z,12Z-octadecatrienoyl)-sn-glycero-3-phospho-(1′-sn-glycerol); 1-(6Z,9Z,12Z-octadecatrienoyl)-2-(9Z,12Z,15Z-octadecatrienoyl)-glycero-3-phospho-(1′-sn-glycerol); 1-(6Z,9Z,12Z-octadecatrienoyl)-2-(6Z,9Z,12Z,15Z-octadecatetraenoyl)-glycero-3-phospho-(1′-sn-glycerol); 1-(6Z,9Z,12Z-octadecatrienoyl)-2-nonadecanoyl-glycero-3-phospho-(1′-sn-glycerol); 1-(6Z,9Z,12Z-octadecatrienoyl)-2-(9Z-nonadecenoyl)-glycero-3-phospho-(1′-sn-glycerol); 1-(6Z,9Z,12Z-octadecatrienoyl)-2-eicosanoyl-glycero-3-phospho-(1′-sn-glycerol); 1-(6Z,9Z,12Z-octadecatrienoyl)-2-(11Z-eicosenoyl)-glycero-3-phospho-(1′-sn-glycerol); 1-(6Z,9Z,12Z-octadecatrienoyl)-2-(11Z,14Z-eicosadienoyl)-glycero-3-phospho-(1′-sn-glycerol); 1-(6Z,9Z,12Z-octadecatrienoyl)-2-(8Z,11Z,14Z-eicosatrienoyl)-glycero-3-phospho-(1′-sn-glycerol); 1-(6Z,9Z,12Z-octadecatrienoyl)-2-(5Z,8Z,11Z,14Z-eicosatetraenoyl)-glycero-3-phospho-(1′-sn-glycerol); 1-(6Z,9Z,12Z-octadecatrienoyl)-2-(5Z,8Z,11Z,14Z,17Z-eicosapentaenoyl)-glycero-3-phospho-(1′-sn-glycerol); 1-(6Z,9Z,12Z-octadecatrienoyl)-2-heneicosanoyl-glycero-3-phospho-(1′-sn-glycerol); 1-(6Z,9Z,12Z-octadecatrienoyl)-2-docosanoyl-glycero-3-phospho-(1′-sn-glycerol); 1-(6Z,9Z,12Z-octadecatrienoyl)-2-(11Z-docosenoyl)-glycero-3-phospho-(1′-sn-glycerol); 1-(6Z,9Z,12Z-octadecatrienoyl)-2-(13Z,16Z-docosadienoyl)-glycero-3-phospho-(1′-sn-glycerol); 1-(6Z,9Z,12Z-octadecatrienoyl)-2-(7Z,10Z,13Z,16Z-docosatetraenoyl)-glycero-3-phospho-(1′-sn-glycerol); 1-(6Z,9Z,12Z-octadecatrienoyl)-2-(4Z,7Z,10Z,13Z,16Z,19Z-docosahexaenoyl)-glycero-3-phospho-(1′-sn-glycerol); 1-(9Z,12Z,15Z-octadecatrienoyl)-2-dodecanoyl-glycero-3-phospho-(1′-sn-glycerol); 1-(9Z,12Z,15Z-octadecatrienoyl)-2-tridecanoyl-glycero-3-phospho-(1′-sn-glycerol); 1-(9Z,12Z,15Z-octadecatrienoyl)-2-tetradecanoyl-glycero-3-phospho-(1′-sn-glycerol); 1-(9Z,12Z,15Z-octadecatrienoyl)-2-(9Z-tetradecenoyl)-glycero-3-phospho-(1′-sn-glycerol); 1-(9Z,12Z,15Z-octadecatrienoyl)-2-pentadecanoyl-glycero-3-phospho-(1′-sn-glycerol); 1-(9Z,12Z,15Z-octadecatrienoyl)-2-(9Z-pentadecenoyl)-glycero-3-phospho-(1′-sn-glycerol); 1-(9Z,12Z,15Z-octadecatrienoyl)-2-(9Z-hexadecenoyl)-glycero-3-phospho-(1′-sn-glycerol); 1-(9Z,12Z,15Z-octadecatrienoyl)-2-heptadecanoyl-glycero-3-phospho-(1′-sn-glycerol); 1-(9Z,12Z,15Z-octadecatrienoyl)-2-(9Z-heptadecenoyl)-glycero-3-phospho-(1′-sn-glycerol); 1-(9Z,12Z,15Z-octadecatrienoyl)-2-(9Z,12Z-heptadecadienoyl)-glycero-3-phospho-(1′-sn-glycerol); 1-(9Z,12Z,15Z-octadecatrienoyl)-2-octadecanoyl-glycero-3-phospho-(1′-sn-glycerol); 1-(9Z,12Z,15Z-octadecatrienoyl)-2-(9Z,12Z-octadecadienoyl)-glycero-3-phospho-(1′-sn-glycerol); 1-(9Z,12Z,15Z-octadecatrienoyl)-2-(6Z,9Z,12Z-octadecatrienoyl)-glycero-3-phospho-(1′-sn-glycerol); 1-(9Z,12Z,15Z-octadecatrienoyl)-2-(6Z,9Z,12Z,15Z-octadecatetraenoyl)-glycero-3-phospho-(1′-sn-glycerol); 1-(9Z,12Z,15Z-octadecatrienoyl)-2-nonadecanoyl-glycero-3-phospho-(1′-sn-glycerol); 1-(9Z,12Z,15Z-octadecatrienoyl)-2-(9Z-nonadecenoyl)-glycero-3-phospho-(1′-sn-glycerol); 1-(9Z,12Z,15Z-octadecatrienoyl)-2-eicosanoyl-glycero-3-phospho-(1′-sn-glycerol); 1-(9Z,12Z,15Z-octadecatrienoyl)-2-(11Z-eicosenoyl)-glycero-3-phospho-(1′-sn-glycerol); 1-(9Z,12Z,15Z-octadecatrienoyl)-2-(11Z,14Z-eicosadienoyl)-glycero-3-phospho-(1′-sn-glycerol); 1-(9Z,12Z,15Z-octadecatrienoyl)-2-(8Z,11Z,14Z-eicosatrienoyl)-glycero-3-phospho-(1′-sn-glycerol); 1-(9Z,12Z,15Z-octadecatrienoyl)-2-(5Z,8Z,11Z,14Z-eicosatetraenoyl)-glycero-3-phospho-(1′-sn-glycerol); 1-(9Z,12Z,15Z-octadecatrienoyl)-2-(5Z,8Z,11Z,14Z,17Z-eicosapentaenoyl)-glycero-3-phospho-(1′-sn-glycerol); 1-(9Z,12Z,15Z-octadecatrienoyl)-2-heneicosanoyl-glycero-3-phospho-(1′-sn-glycerol); 1-(9Z,12Z,15Z-octadecatrienoyl)-2-docosanoyl-glycero-3-phospho-(1′-sn-glycerol); 1-(9Z,12Z,15Z-octadecatrienoyl)-2-(11Z-docosenoyl)-glycero-3-phospho-(1′-sn-glycerol); 1-(9Z,12Z,15Z-octadecatrienoyl)-2-(13Z,16Z-docosadienoyl)-glycero-3-phospho-(1′-sn-glycerol); 1-(9Z,12Z,15Z-octadecatrienoyl)-2-(7Z,10Z,13Z,16Z-docosatetraenoyl)-glycero-3-phospho-(1′-sn-glycerol); 1-(9Z,12Z,15Z-octadecatrienoyl)-2-(4Z,7Z,10Z,13Z,16Z,19Z-docosahexaenoyl)-glycero-3-phospho-(1′-sn-glycerol); 1-(6Z,9Z,12Z,15Z-octadecatetraenoyl)-glycero-3-phospho-(1′-sn-glycerol); 1-(6Z,9Z,12Z,15Z-octadecatetraenoyl)-2-dodecanoyl-glycero-3-phospho-(1′-sn-glycerol); 1-(6Z,9Z,12Z,15Z-octadecatetraenoyl)-2-tridecanoyl-glycero-3-phospho-(1′-sn-glycerol); 1-(6Z,9Z,12Z,15Z-octadecatetraenoyl)-2-tetradecanoyl-glycero-3-phospho-(1′-sn-glycerol); 1-(6Z,9Z,12Z,15Z-octadecatetraenoyl)-2-(9Z-tetradecenoyl)-glycero-3-phospho-(1′-sn-glycerol); 1-(6Z,9Z,12Z,15Z-octadecatetraenoyl)-2-pentadecanoyl-glycero-3-phospho-(1′-sn-glycerol); 1-(6Z,9Z,12Z,15Z-octadecatetraenoyl)-2-(9Z-pentadecenoyl)-glycero-3-phospho-(1′-sn-glycerol); 1-(6Z,9Z,12Z,15Z-octadecatetraenoyl)-2-hexadecanoyl-glycero-3-phospho-(1′-sn-glycerol); 1-(6Z,9Z,12Z,15Z-octadecatetraenoyl)-2-(9Z-hexadecenoyl)-glycero-3-phospho-(1′-sn-glycerol); 1-(6Z,9Z,12Z,15Z-octadecatetraenoyl)-2-heptadecanoyl-glycero-3-phospho-(1′-sn-glycerol); 1-(6Z,9Z,12Z,15Z-octadecatetraenoyl)-2-(9Z-heptadecenoyl)-glycero-3-phospho-(1′-sn-glycerol); 1-(6Z,9Z,12Z,15Z-octadecatetraenoyl)-2-(9Z,12Z-heptadecadienoyl)-glycero-3-phospho-(1′-sn-glycerol); 1-(6Z,9Z,12Z,15Z-octadecatetraenoyl)-2-octadecanoyl-glycero-3-phospho-(1′-sn-glycerol); 1-(6Z,9Z,12Z,15Z-octadecatetraenoyl)-2-(9Z-octadecenoyl)-glycero-3-phospho-(1′-sn-glycerol); 1-(6Z,9Z,12Z,15Z-octadecatetraenoyl)-2-(9Z,12Z-octadecadienoyl)-glycero-3-phospho-(1′-sn-glycerol); 1-(6Z,9Z,12Z,15Z-octadecatetraenoyl)-2-(6Z,9Z,12Z-octadecatrienoyl)-glycero-3-phospho-(1′-sn-glycerol); 1-(6Z,9Z,12Z,15Z-octadecatetraenoyl)-2-(9Z,12Z,15Z-octadecatrienoyl)-glycero-3-phospho-(1′-sn-glycerol); 1,2-di-(6Z,9Z,12Z,15Z-octadecatetraenoyl)-sn-glycero-3-phospho-(1′-sn-glycerol); 1-(6Z,9Z,12Z,15Z-octadecatetraenoyl)-2-nonadecanoyl-glycero-3-phospho-(1′-sn-glycerol); 1-(6Z,9Z,12Z,15Z-octadecatetraenoyl)-2-(9Z-nonadecenoyl)-glycero-3-phospho-(1′-sn-glycerol); 1-(6Z,9Z,12Z,15Z-octadecatetraenoyl)-2-eicosanoyl-glycero-3-phospho-(1′-sn-glycerol); 1-(6Z,9Z,12Z,15Z-octadecatetraenoyl)-2-(11Z-eicosenoyl)-glycero-3-phospho-(1′-sn-glycerol); 1-(6Z,9Z,12Z,15Z-octadecatetraenoyl)-2-(11Z,14Z-eicosadienoyl)-glycero-3-phospho-(1′-sn-glycerol); 1-(6Z,9Z,12Z,15Z-octadecatetraenoyl)-2-(8Z,11Z,14Z-eicosatrienoyl)-glycero-3-phospho-(1′-sn-glycerol); 1-(6Z,9Z,12Z,15Z-octadecatetraenoyl)-2-(5Z,8Z,11Z,14Z-eicosatetraenoyl)-glycero-3-phospho-(1′-sn-glycerol); 1-(6Z,9Z,12Z,15Z-octadecatetraenoyl)-2-(5Z,8Z,11Z,14Z,17Z-eicosapentaenoyl)-glycero-3-phospho-(1′-sn-glycerol); 1-(6Z,9Z,12Z,15Z-octadecatetraenoyl)-2-heneicosanoyl-glycero-3-phospho-(1′-sn-glycerol); 1-(6Z,9Z,12Z,15Z-octadecatetraenoyl)-2-docosanoyl-glycero-3-phospho-(1′-sn-glycerol); 1-(6Z,9Z,12Z,15Z-octadecatetraenoyl)-2-(11Z-docosenoyl)-glycero-3-phospho-(1′-sn-glycerol); 1-(6Z,9Z,12Z,15Z-octadecatetraenoyl)-2-(13Z,16Z-docosadienoyl)-glycero-3-phospho-(1′-sn-glycerol); 1-(6Z,9Z,12Z,15Z-octadecatetraenoyl)-2-(7Z,10Z,13Z,16Z-docosatetraenoyl)-glycero-3-phospho-(1′-sn-glycerol); 1-(6Z,9Z,12Z,15Z-octadecatetraenoyl)-2-(4Z,7Z,10Z,13Z,16Z,19Z-docosahexaenoyl)-glycero-3-phospho-(1′-sn-glycerol); 1-nonadecanoyl-2-(9Z-tetradecenoyl)-glycero-3-phospho-(1′-sn-glycerol); 1-nonadecanoyl-2-(9Z-pentadecenoyl)-glycero-3-phospho-(1′-sn-glycerol); 1-nonadecanoyl-2-(9Z-hexadecenoyl)-glycero-3-phospho-(1′-sn-glycerol); 1-nonadecanoyl-2-heptadecanoyl-glycero-3-phospho-(1′-sn-glycerol); 1-nonadecanoyl-2-(9Z-heptadecenoyl)-glycero-3-phospho-(1′-sn-glycerol); 1-nonadecanoyl-2-(9Z,12Z-heptadecadienoyl)-glycero-3-phospho-(1′-sn-glycerol); 1-nonadecanoyl-2-(9Z-octadecenoyl)-glycero-3-phospho-(1′-sn-glycerol); 1-nonadecanoyl-2-(9Z,12Z-octadecadienoyl)-glycero-3-phospho-(1′-sn-glycerol); 1-nonadecanoyl-2-(6Z,9Z,12Z-octadecatrienoyl)-glycero-3-phospho-(1′-sn-glycerol); 1-nonadecanoyl-2-(9Z,12Z,15Z-octadecatrienoyl)-glycero-3-phospho-(1′-sn-glycerol); 1-nonadecanoyl-2-(6Z,9Z,12Z,15Z-octadecatetraenoyl)-glycero-3-phospho-(1′-sn-glycerol); 1-nonadecanoyl-2-(9Z-nonadecenoyl)-glycero-3-phospho-(1′-sn-glycerol); 1-nonadecanoyl-2-eicosanoyl-glycero-3-phospho-(1′-sn-glycerol); 1-nonadecanoyl-2-(11Z-eicosenoyl)-glycero-3-phospho-(1′-sn-glycerol); 1-nonadecanoyl-2-(11Z,14Z-eicosadienoyl)-glycero-3-phospho-(1′-sn-glycerol); 1-nonadecanoyl-2-(8Z,11Z,14Z-eicosatrienoyl)-glycero-3-phospho-(1′-sn-glycerol); 1-nonadecanoyl-2-(5Z,8Z,11Z,14Z-eicosatetraenoyl)-glycero-3-phospho-(1′-sn-glycerol); 1-nonadecanoyl-2-(5Z,8Z,11Z,14Z,17Z-eicosapentaenoyl)-glycero-3-phospho-(1′-sn-glycerol); 1-nonadecanoyl-2-heneicosanoyl-glycero-3-phospho-(1′-sn-glycerol); 1-nonadecanoyl-2-docosanoyl-glycero-3-phospho-(1′-sn-glycerol); 1-nonadecanoyl-2-(11Z-docosenoyl)-glycero-3-phospho-(1′-sn-glycerol); 1-nonadecanoyl-2-(13Z,16Z-docosadienoyl)-glycero-3-phospho-(1′-sn-glycerol); 1-nonadecanoyl-2-(7Z,10Z,13Z,16Z-docosatetraenoyl)-glycero-3-phospho-(1′-sn-glycerol); 1-nonadecanoyl-2-(4Z,7Z,10Z,13Z,16Z,19Z-docosahexaenoyl)-glycero-3-phospho-(1′-sn-glycerol); 1-(9Z-nonadecenoyl)-glycero-3-phospho-(1′-sn-glycerol); 1-(9Z-nonadecenoyl)-2-dodecanoyl-glycero-3-phospho-(1′-sn-glycerol); 1-(9Z-nonadecenoyl)-2-tridecanoyl-glycero-3-phospho-(1′-sn-glycerol); 1-(9Z-nonadecenoyl)-2-tetradecanoyl-glycero-3-phospho-(1′-sn-glycerol); 1-(9Z-nonadecenoyl)-2-(9Z-tetradecenoyl)-glycero-3-phospho-(1′-sn-glycerol); 1-(9Z-nonadecenoyl)-2-pentadecanoyl-glycero-3-phospho-(1′-sn-glycerol); 1-(9Z-nonadecenoyl)-2-(9Z-pentadecenoyl)-glycero-3-phospho-(1′-sn-glycerol); 1-(9Z-nonadecenoyl)-2-hexadecanoyl-glycero-3-phospho-(1′-sn-glycerol); 1-(9Z-nonadecenoyl)-2-(9Z-hexadecenoyl)-glycero-3-phospho-(1′-sn-glycerol); 1-(9Z-nonadecenoyl)-2-heptadecanoyl-glycero-3-phospho-(1′-sn-glycerol); 1-(9Z-nonadecenoyl)-2-(9Z-heptadecenoyl)-glycero-3-phospho-(1′-sn-glycerol); 1-(9Z-nonadecenoyl)-2-(9Z,12Z-heptadecadienoyl)-glycero-3-phospho-(1′-sn-glycerol); 1-(9Z-nonadecenoyl)-2-octadecanoyl-glycero-3-phospho-(1′-sn-glycerol); 1-(9Z-nonadecenoyl)-2-(9Z-octadecenoyl)-glycero-3-phospho-(1′-sn-glycerol); 1-(9Z-nonadecenoyl)-2-(9Z,12Z-octadecadienoyl)-glycero-3-phospho-(1′-sn-glycerol); 1-(9Z-nonadecenoyl)-2-(6Z,9Z,12Z-octadecatrienoyl)-glycero-3-phospho-(1′-sn-glycerol); 1-(9Z-nonadecenoyl)-2-(9Z,12Z,15Z-octadecatrienoyl)-glycero-3-phospho-(1′-sn-glycerol); 1-(9Z-nonadecenoyl)-2-(6Z,9Z,12Z,15Z-octadecatetraenoyl)-glycero-3-phospho-(1′-sn-glycerol); 1-(9Z-nonadecenoyl)-2-nonadecanoyl-glycero-3-phospho-(1′-sn-glycerol); 1,2-di-(9Z-nonadecenoyl)-sn-glycero-3-phospho-(1′-sn-glycerol); 1-(9Z-nonadecenoyl)-2-eicosanoyl-glycero-3-phospho-(1′-sn-glycerol); 1-(9Z-nonadecenoyl)-2-(11Z-eicosenoyl)-glycero-3-phospho-(1′-sn-glycerol); 1-(9Z-nonadecenoyl)-2-(11Z,14Z-eicosadienoyl)-glycero-3-phospho-(1′-sn-glycerol); 1-(9Z-nonadecenoyl)-2-(8Z,11Z,14Z-eicosatrienoyl)-glycero-3-phospho-(1′-sn-glycerol); 1-(9Z-nonadecenoyl)-2-(5Z,8Z,11Z,14Z-eicosatetraenoyl)-glycero-3-phospho-(1′-sn-glycerol); 1-(9Z-nonadecenoyl)-2-(5Z,8Z,11Z,14Z,17Z-eicosapentaenoyl)-glycero-3-phospho-(1′-sn-glycerol); 1-(9Z-nonadecenoyl)-2-heneicosanoyl-glycero-3-phospho-(1′-sn-glycerol); 1-(9Z-nonadecenoyl)-2-docosanoyl-glycero-3-phospho-(1′-sn-glycerol); 1-(9Z-nonadecenoyl)-2-(11Z-docosenoyl)-glycero-3-phospho-(1′-sn-glycerol); 1-(9Z-nonadecenoyl)-2-(13Z,16Z-docosadienoyl)-glycero-3-phospho-(1′-sn-glycerol); 1-(9Z-nonadecenoyl)-2-(7Z,10Z,13Z,16Z-docosatetraenoyl)-glycero-3-phospho-(1′-sn-glycerol); 1-(9Z-nonadecenoyl)-2-(4Z,7Z,10Z,13Z,16Z,19Z-docosahexaenoyl)-glycero-3-phospho-(1′-sn-glycerol); 1-eicosanoyl-2-(9Z-tetradecenoyl)-glycero-3-phospho-(1′-sn-glycerol); 1-eicosanoyl-2-pentadecanoyl-glycero-3-phospho-(1′-sn-glycerol); 1-eicosanoyl-2-(9Z-pentadecenoyl)-glycero-3-phospho-(1′-sn-glycerol); 1-eicosanoyl-2-(9Z-hexadecenoyl)-glycero-3-phospho-(1′-sn-glycerol); 1-eicosanoyl-2-heptadecanoyl-glycero-3-phospho-(1′-sn-glycerol); 1-eicosanoyl-2-(9Z-heptadecenoyl)-glycero-3-phospho-(1′-sn-glycerol); 1-eicosanoyl-2-(9Z,12Z-heptadecadienoyl)-glycero-3-phospho-(1′-sn-glycerol); 1-eicosanoyl-2-(6Z,9Z,12Z-octadecatrienoyl)-glycero-3-phospho-(1′-sn-glycerol); 1-eicosanoyl-2-(9Z,12Z,15Z-octadecatrienoyl)-glycero-3-phospho-(1′-sn-glycerol); 1-eicosanoyl-2-nonadecanoyl-glycero-3-phospho-(1′-sn-glycerol); 1-eicosanoyl-2-(9Z-nonadecenoyl)-glycero-3-phospho-(1′-sn-glycerol); 1-eicosanoyl-2-(8Z,11Z,14Z-eicosatrienoyl)-glycero-3-phospho-(1′-sn-glycerol); 1-eicosanoyl-2-(5Z,8Z,11Z,14Z,17Z-eicosapentaenoyl)-glycero-3-phospho-(1′-sn-glycerol); 1-eicosanoyl-2-heneicosanoyl-glycero-3-phospho-(1′-sn-glycerol); 1-eicosanoyl-2-(11Z-docosenoyl)-glycero-3-phospho-(1′-sn-glycerol); 1-eicosanoyl-2-(13Z,16Z-docosadienoyl)-glycero-3-phospho-(1′-sn-glycerol); 1-eicosanoyl-2-(7Z,10Z,13Z,16Z-docosatetraenoyl)-glycero-3-phospho-(1′-sn-glycerol); 1-(11Z-eicosenoyl)-glycero-3-phospho-(1′-sn-glycerol); 1-(11Z-eicosenoyl)-2-dodecanoyl-glycero-3-phospho-(1′-sn-glycerol); 1-(11Z-eicosenoyl)-2-tridecanoyl-glycero-3-phospho-(1′-sn-glycerol); 1-(11Z-eicosenoyl)-2-tetradecanoyl-glycero-3-phospho-(1′-sn-glycerol); 1-(11Z-eicosenoyl)-2-(9Z-tetradecenoyl)-glycero-3-phospho-(1′-sn-glycerol); 1-(11Z-eicosenoyl)-2-pentadecanoyl-glycero-3-phospho-(1′-sn-glycerol); 1-(11Z-eicosenoyl)-2-(9Z-pentadecenoyl)-glycero-3-phospho-(1′-sn-glycerol); 1-(11Z-eicosenoyl)-2-hexadecanoyl-glycero-3-phospho-(1′-sn-glycerol); 1-(11Z-eicosenoyl)-2-(9Z-hexadecenoyl)-glycero-3-phospho-(1′-sn-glycerol); 1-(11Z-eicosenoyl)-2-heptadecanoyl-glycero-3-phospho-(1′-sn-glycerol); 1-(11Z-eicosenoyl)-2-(9Z-heptadecenoyl)-glycero-3-phospho-(1′-sn-glycerol); 1-(11Z-eicosenoyl)-2-(9Z,12Z-heptadecadienoyl)-glycero-3-phospho-(1′-sn-glycerol); 1-(11Z-eicosenoyl)-2-octadecanoyl-glycero-3-phospho-(1′-sn-glycerol); 1-(11Z-eicosenoyl)-2-(9Z-octadecenoyl)-glycero-3-phospho-(1′-sn-glycerol); 1-(11Z-eicosenoyl)-2-(9Z,12Z-octadecadienoyl)-glycero-3-phospho-(1′-sn-glycerol); 1-(11Z-eicosenoyl)-2-(6Z,9Z,12Z-octadecatrienoyl)-glycero-3-phospho-(1′-sn-glycerol); 1-(11Z-eicosenoyl)-2-(9Z,12Z,15Z-octadecatrienoyl)-glycero-3-phospho-(1′-sn-glycerol); 1-(11Z-eicosenoyl)-2-(6Z,9Z,12Z,15Z-octadecatetraenoyl)-glycero-3-phospho-(1′-sn-glycerol); 1-(11Z-eicosenoyl)-2-nonadecanoyl-glycero-3-phospho-(1′-sn-glycerol); 1-(11Z-eicosenoyl)-2-(9Z-nonadecenoyl)-glycero-3-phospho-(1′-sn-glycerol); 1-(11Z-eicosenoyl)-2-eicosanoyl-glycero-3-phospho-(1′-sn-glycerol); 1-(11Z-eicosenoyl)-2-(11Z,14Z-eicosadienoyl)-glycero-3-phospho-(1′-sn-glycerol); 1-(11Z-eicosenoyl)-2-(8Z,11Z,14Z-eicosatrienoyl)-glycero-3-phospho-(1′-sn-glycerol); 1-(11Z-eicosenoyl)-2-(5Z,8Z,11Z,14Z-eicosatetraenoyl)-glycero-3-phospho-(1′-sn-glycerol); 1-(11Z-eicosenoyl)-2-(5Z,8Z,11Z,14Z,17Z-eicosapentaenoyl)-glycero-3-phospho-(1′-sn-glycerol); 1-(11Z-eicosenoyl)-2-heneicosanoyl-glycero-3-phospho-(1′-sn-glycerol); 1-(11 Z-eicosenoyl)-2-docosanoyl-glycero-3-phospho-(1′-sn-glycerol); 1-(11Z-eicosenoyl)-2-(11Z-docosenoyl)-glycero-3-phospho-(1′-sn-glycerol); 1-(11Z-eicosenoyl)-2-(13Z,16Z-docosadienoyl)-glycero-3-phospho-(1′-sn-glycerol); 1-(11Z-eicosenoyl)-2-(7Z,10Z,13Z,16Z-docosatetraenoyl)-glycero-3-phospho-(1′-sn-glycerol); 1-(11Z-eicosenoyl)-2-(4Z,7Z,10Z,13Z,16Z,19Z-docosahexaenoyl)-glycero-3-phospho-(1′-sn-glycerol); 1-(11Z,14Z-eicosadienoyl)-glycero-3-phospho-(1′-sn-glycerol); 1-(11Z,14Z-eicosadienoyl)-2-dodecanoyl-glycero-3-phospho-(1′-sn-glycerol); 1-(11Z,14Z-eicosadienoyl)-2-tridecanoyl-glycero-3-phospho-(1′-sn-glycerol); 1-(11Z,14Z-eicosadienoyl)-2-tetradecanoyl-glycero-3-phospho-(1′-sn-glycerol); 1-(11Z,14Z-eicosadienoyl)-2-(9Z-tetradecenoyl)-glycero-3-phospho-(1′-sn-glycerol); 1-(11Z,14Z-eicosadienoyl)-2-pentadecanoyl-glycero-3-phospho-(1′-sn-glycerol); 1-(11Z,14Z-eicosadienoyl)-2-(9Z-pentadecenoyl)-glycero-3-phospho-(1′-sn-glycerol); 1-(11Z,14Z-eicosadienoyl)-2-hexadecanoyl-glycero-3-phospho-(1′-sn-glycerol); 1-(11Z,14Z-eicosadienoyl)-2-(9Z-hexadecenoyl)-glycero-3-phospho-(1′-sn-glycerol); 1-(11Z,14Z-eicosadienoyl)-2-heptadecanoyl-glycero-3-phospho-(1′-sn-glycerol); 1-(11Z,14Z-eicosadienoyl)-2-(9Z-heptadecenoyl)-glycero-3-phospho-(1′-sn-glycerol); 1-(11Z,14Z-eicosadienoyl)-2-(9Z,12Z-heptadecadienoyl)-glycero-3-phospho-(1′-sn-glycerol); 1-(11Z,14Z-eicosadienoyl)-2-octadecanoyl-glycero-3-phospho-(1′-sn-glycerol); 1-(11Z,14Z-eicosadienoyl)-2-(9Z-octadecenoyl)-glycero-3-phospho-(1′-sn-glycerol); 1-(11Z,14Z-eicosadienoyl)-2-(9Z,12Z-octadecadienoyl)-glycero-3-phospho-(1′-sn-glycerol); 1-(11Z,14Z-eicosadienoyl)-2-(6Z,9Z,12Z-octadecatrienoyl)-glycero-3-phospho-(1′-sn-glycerol); 1-(11Z,14Z-eicosadienoyl)-2-(9Z,12Z,15Z-octadecatrienoyl)-glycero-3-phospho-(1′-sn-glycerol); 1-(11Z,14Z-eicosadienoyl)-2-(6Z,9Z,12Z,15Z-octadecatetraenoyl)-glycero-3-phospho-(1′-sn-glycerol); 1-(11Z,14Z-eicosadienoyl)-2-nonadecanoyl-glycero-3-phospho-(1′-sn-glycerol); 1-(11Z,14Z-eicosadienoyl)-2-(9Z-nonadecenoyl)-glycero-3-phospho-(1′-sn-glycerol); 1-(11Z,14Z-eicosadienoyl)-2-eicosanoyl-glycero-3-phospho-(1′-sn-glycerol); 1-(11Z,14Z-eicosadienoyl)-2-(11Z-eicosenoyl)-glycero-3-phospho-(1′-sn-glycerol); 1,2-di-(11Z,14Z-eicosadienoyl)-sn-glycero-3-phospho-(1′-sn-glycerol); 1-(11Z,14Z-eicosadienoyl)-2-(8Z,11Z,14Z-eicosatrienoyl)-glycero-3-phospho-(1′-sn-glycerol); 1-(11Z,14Z-eicosadienoyl)-2-(5Z,8Z,11Z,14Z-eicosatetraenoyl)-glycero-3-phospho-(1′-sn-glycerol); 1-(11Z,14Z-eicosadienoyl)-2-(5Z,8Z,11Z,14Z,17Z-eicosapentaenoyl)-glycero-3-phospho-(1′-sn-glycerol); 1-(11Z,14Z-eicosadienoyl)-2-heneicosanoyl-glycero-3-phospho-(1′-sn-glycerol); 1-(11Z,14Z-eicosadienoyl)-2-docosanoyl-glycero-3-phospho-(1′-sn-glycerol); 1-(11Z,14Z-eicosadienoyl)-2-(11Z-docosenoyl)-glycero-3-phospho-(1′-sn-glycerol); 1-(11Z,14Z-eicosadienoyl)-2-(13Z,16Z-docosadienoyl)-glycero-3-phospho-(1′-sn-glycerol); 1-(11Z,14Z-eicosadienoyl)-2-(7Z,10Z,13Z,16Z-docosatetraenoyl)-glycero-3-phospho-(1′-sn-glycerol); 1-(11Z,14Z-eicosadienoyl)-2-(4Z,7Z,10Z,13Z,16Z,19Z-docosahexaenoyl)-glycero-3-phospho-(1′-sn-glycerol); 1-(8Z,11Z,14Z-eicosatrienoyl)-glycero-3-phospho-(1′-sn-glycerol); 1-(8Z,11Z,14Z-eicosatrienoyl)-2-dodecanoyl-glycero-3-phospho-(1′-sn-glycerol); 1-(8Z,11Z,14Z-eicosatrienoyl)-2-tridecanoyl-glycero-3-phospho-(1′-sn-glycerol); 1-(8Z,11Z,14Z-eicosatrienoyl)-2-tetradecanoyl-glycero-3-phospho-(1′-sn-glycerol); 1-(8Z,11Z,14Z-eicosatrienoyl)-2-(9Z-tetradecenoyl)-glycero-3-phospho-(1′-sn-glycerol); 1-(8Z,11Z,14Z-eicosatrienoyl)-2-pentadecanoyl-glycero-3-phospho-(1′-sn-glycerol); 1-(8Z,11Z,14Z-eicosatrienoyl)-2-(9Z-pentadecenoyl)-glycero-3-phospho-(1′-sn-glycerol); 1-(8Z,11Z,14Z-eicosatrienoyl)-2-hexadecanoyl-glycero-3-phospho-(1′-sn-glycerol); 1-(8Z,11Z,14Z-eicosatrienoyl)-2-(9Z-hexadecenoyl)-glycero-3-phospho-(1′-sn-glycerol); 1-(8Z,11Z,14Z-eicosatrienoyl)-2-heptadecanoyl-glycero-3-phospho-(1′-sn-glycerol); 1-(8Z,11Z,14Z-eicosatrienoyl)-2-(9Z-heptadecenoyl)-glycero-3-phospho-(1′-sn-glycerol); 1-(8Z,11Z,14Z-eicosatrienoyl)-2-(9Z,12Z-heptadecadienoyl)-glycero-3-phospho-(1′-sn-glycerol); 1-(8Z,11Z,14Z-eicosatrienoyl)-2-octadecanoyl-glycero-3-phospho-(1′-sn-glycerol); 1-(8Z,11Z,14Z-eicosatrienoyl)-2-(9Z-octadecenoyl)-glycero-3-phospho-(1′-sn-glycerol); 1-(8Z,11Z,14Z-eicosatrienoyl)-2-(9Z,12Z-octadecadienoyl)-glycero-3-phospho-(1′-sn-glycerol); 1-(8Z,11Z,14Z-eicosatrienoyl)-2-(6Z,9Z,12Z-octadecatrienoyl)-glycero-3-phospho-(1′-sn-glycerol); 1-(8Z,11Z,14Z-eicosatrienoyl)-2-(9Z,12Z,15Z-octadecatrienoyl)-glycero-3-phospho-(1′-sn-glycerol); 1-(8Z,11Z,14Z-eicosatrienoyl)-2-(6Z,9Z,12Z,15Z-octadecatetraenoyl)-glycero-3-phospho-(1′-sn-glycerol); 1-(8Z,11Z,14Z-eicosatrienoyl)-2-nonadecanoyl-glycero-3-phospho-(1′-sn-glycerol); 1-(8Z,11Z,14Z-eicosatrienoyl)-2-(9Z-nonadecenoyl)-glycero-3-phospho-(1′-sn-glycerol); 1-(8Z,11Z,14Z-eicosatrienoyl)-2-eicosanoyl-glycero-3-phospho-(1′-sn-glycerol); 1-(8Z,11Z,14Z-eicosatrienoyl)-2-(11Z-eicosenoyl)-glycero-3-phospho-(1′-sn-glycerol); 1-(8Z,11Z,14Z-eicosatrienoyl)-2-(11Z,14Z-eicosadienoyl)-glycero-3-phospho-(1′-sn-glycerol); 1,2-di-(8Z,11Z,14Z-eicosatrienoyl)-sn-glycero-3-phospho-(1′-sn-glycerol); 1-(8Z,11Z,14Z-eicosatrienoyl)-2-(5Z,8Z,11Z,14Z-eicosatetraenoyl)-glycero-3-phospho-(1′-sn-glycerol); 1-(8Z,11Z,14Z-eicosatrienoyl)-2-(5Z,8Z,11Z,14Z,17Z-eicosapentaenoyl)-glycero-3-phospho-(1′-sn-glycerol); 1-(8Z,11Z,14Z-eicosatrienoyl)-2-heneicosanoyl-glycero-3-phospho-(1′-sn-glycerol); 1-(8Z,11Z,14Z-eicosatrienoyl)-2-docosanoyl-glycero-3-phospho-(1′-sn-glycerol); 1-(8Z,11Z,14Z-eicosatrienoyl)-2-(11Z-docosenoyl)-glycero-3-phospho-(1′-sn-glycerol); 1-(8Z,11Z,14Z-eicosatrienoyl)-2-(13Z,16Z-docosadienoyl)-glycero-3-phospho-(1′-sn-glycerol); 1-(8Z,11Z,14Z-eicosatrienoyl)-2-(7Z,10Z,13Z,16Z-docosatetraenoyl)-glycero-3-phospho-(1′-sn-glycerol); 1-(8Z,11Z,14Z-eicosatrienoyl)-2-(4Z,7Z,10Z,13Z,16Z,19Z-docosahexaenoyl)-glycero-3-phospho-(1′-sn-glycerol); 1-(5Z,8Z,11Z,14Z-eicosatetraenoyl)-2-dodecanoyl-glycero-3-phospho-(1′-sn-glycerol); 1-(5Z,8Z,11Z,14Z-eicosatetraenoyl)-2-tridecanoyl-glycero-3-phospho-(1′-sn-glycerol); 1-(5Z,8Z,11Z,14Z-eicosatetraenoyl)-2-tetradecanoyl-glycero-3-phospho-(1′-sn-glycerol); 1-(5Z,8Z,11Z,14Z-eicosatetraenoyl)-2-(9Z-tetradecenoyl)-glycero-3-phospho-(1′-sn-glycerol); 1-(5Z,8Z,11Z,14Z-eicosatetraenoyl)-2-pentadecanoyl-glycero-3-phospho-(1′-sn-glycerol); 1-(5Z,8Z,11Z,14Z-eicosatetraenoyl)-2-(9Z-pentadecenoyl)-glycero-3-phospho-(1′-sn-glycerol); 1-(5Z,8Z,11Z,14Z-eicosatetraenoyl)-2-(9Z-hexadecenoyl)-glycero-3-phospho-(1′-sn-glycerol); 1-(5Z,8Z,11Z,14Z-eicosatetraenoyl)-2-heptadecanoyl-glycero-3-phospho-(1′-sn-glycerol); 1-(5Z,8Z,11Z,14Z-eicosatetraenoyl)-2-(9Z-heptadecenoyl)-glycero-3-phospho-(1′-sn-glycerol); 1-(5Z,8Z,11Z,14Z-eicosatetraenoyl)-2-(9Z,12Z-heptadecadienoyl)-glycero-3-phospho-(1′-sn-glycerol); 1-(5Z,8Z,11Z,14Z-eicosatetraenoyl)-2-octadecanoyl-glycero-3-phospho-(1′-sn-glycerol); 1-(5Z,8Z,11Z,14Z-eicosatetraenoyl)-2-(9Z-octadecenoyl)-glycero-3-phospho-(1′-sn-glycerol); 1-(5Z,8Z,11Z,14Z-eicosatetraenoyl)-2-(9Z,12Z-octadecadienoyl)-glycero-3-phospho-(1′-sn-glycerol); 1-(5Z,8Z,11Z,14Z-eicosatetraenoyl)-2-(6Z,9Z,12Z-octadecatrienoyl)-glycero-3-phospho-(1′-sn-glycerol); 1-(5Z,8Z,11Z,14Z-eicosatetraenoyl)-2-(9Z,12Z,15Z-octadecatrienoyl)-glycero-3-phospho-(1′-sn-glycerol); 1-(5Z,8Z,11Z,14Z-eicosatetraenoyl)-2-(6Z,9Z,12Z,15Z-octadecatetraenoyl)-glycero-3-phospho-(1′-sn-glycerol); 1-(5Z,8Z,11Z,14Z-eicosatetraenoyl)-2-nonadecanoyl-glycero-3-phospho-(1′-sn-glycerol); 1-(5Z,8Z,11Z,14Z-eicosatetraenoyl)-2-(9Z-nonadecenoyl)-glycero-3-phospho-(1′-sn-glycerol); 1-(5Z,8Z,11Z,14Z-eicosatetraenoyl)-2-eicosanoyl-glycero-3-phospho-(1′-sn-glycerol); 1-(5Z,8Z,11Z,14Z-eicosatetraenoyl)-2-(11Z-eicosenoyl)-glycero-3-phospho-(1′-sn-glycerol); 1-(5Z,8Z,11Z,14Z-eicosatetraenoyl)-2-(11Z,14Z-eicosadienoyl)-glycero-3-phospho-(1′-sn-glycerol); 1-(5Z,8Z,11Z,14Z-eicosatetraenoyl)-2-(8Z,11Z,14Z-eicosatrienoyl)-glycero-3-phospho-(1′-sn-glycerol); 1-(5Z,8Z,11Z,14Z-eicosatetraenoyl)-2-(5Z,8Z,11Z,14Z,17Z-eicosapentaenoyl)-glycero-3-phospho-(1′-sn-glycerol); 1-(5Z,8Z,11Z,14Z-eicosatetraenoyl)-2-heneicosanoyl-glycero-3-phospho-(1′-sn-glycerol); 1-(5Z,8Z,11Z,14Z-eicosatetraenoyl)-2-docosanoyl-glycero-3-phospho-(1′-sn-glycerol); 1-(5Z,8Z,11Z,14Z-eicosatetraenoyl)-2-(11Z-docosenoyl)-glycero-3-phospho-(1′-sn-glycerol); 1-(5Z,8Z,11Z,14Z-eicosatetraenoyl)-2-(13Z,16Z-docosadienoyl)-glycero-3-phospho-(1′-sn-glycerol); 1-(5Z,8Z,11Z,14Z-eicosatetraenoyl)-2-(7Z,10Z,13Z,16Z-docosatetraenoyl)-glycero-3-phospho-(1′-sn-glycerol); 1-(5Z,8Z,11Z,14Z,17Z-eicosapentaenoyl)-2-dodecanoyl-glycero-3-phospho-(1′-sn-glycerol); 1-(5Z,8Z,11Z,14Z,17Z-eicosapentaenoyl)-2-tridecanoyl-glycero-3-phospho-(1′-sn-glycerol); 1-(5Z,8Z,11Z,14Z,17Z-eicosapentaenoyl)-2-tetradecanoyl-glycero-3-phospho-(1′-sn-glycerol); 1-(5Z,8Z,11Z,14Z,17Z-eicosapentaenoyl)-2-(9Z-tetradecenoyl)-glycero-3-phospho-(1′-sn-glycerol); 1-(5Z,8Z,11Z,14Z,17Z-eicosapentaenoyl)-2-pentadecanoyl-glycero-3-phospho-(1′-sn-glycerol); 1-(5Z,8Z,11Z,14Z,17Z-eicosapentaenoyl)-2-(9Z-pentadecenoyl)-glycero-3-phospho-(1′-sn-glycerol); 1-(5Z,8Z,11Z,14Z,17Z-eicosapentaenoyl)-2-hexadecanoyl-glycero-3-phospho-(1′-sn-glycerol); 1-(5Z,8Z,11Z,14Z,17Z-eicosapentaenoyl)-2-(9Z-hexadecenoyl)-glycero-3-phospho-(1′-sn-glycerol); 1-(5Z,8Z,11Z,14Z,17Z-eicosapentaenoyl)-2-heptadecanoyl-glycero-3-phospho-(1′-sn-glycerol); 1-(5Z,8Z,11Z,14Z,17Z-eicosapentaenoyl)-2-(9Z-heptadecenoyl)-glycero-3-phospho-(1′-sn-glycerol); 1-(5Z,8Z,11Z,14Z,17Z-eicosapentaenoyl)-2-(9Z,12Z-heptadecadienoyl)-glycero-3-phospho-(1′-sn-glycerol); 1-(5Z,8Z,11Z,14Z,17Z-eicosapentaenoyl)-2-octadecanoyl-glycero-3-phospho-(1′-sn-glycerol); 1-(5Z,8Z,11Z,14Z,17Z-eicosapentaenoyl)-2-(9Z,12Z-octadecadienoyl)-glycero-3-phospho-(1′-sn-glycerol); 1-(5Z,8Z,11Z,14Z,17Z-eicosapentaenoyl)-2-(6Z,9Z,12Z-octadecatrienoyl)-glycero-3-phospho-(1′-sn-glycerol); 1-(5Z,8Z,11Z,14Z,17Z-eicosapentaenoyl)-2-(9Z,12Z,15Z-octadecatrienoyl)-glycero-3-phospho-(1′-sn-glycerol); 1-(5Z,8Z,11Z,14Z,17Z-eicosapentaenoyl)-2-(6Z,9Z,12Z,15Z-octadecatetraenoyl)-glycero-3-phospho-(1′-sn-glycerol); 1-(5Z,8Z,11Z,14Z,17Z-eicosapentaenoyl)-2-nonadecanoyl-glycero-3-phospho-(1′-sn-glycerol); 1-(5Z,8Z,11Z,14Z,17Z-eicosapentaenoyl)-2-(9Z-nonadecenoyl)-glycero-3-phospho-(1′-sn-glycerol); 1-(5Z,8Z,11Z,14Z,17Z-eicosapentaenoyl)-2-eicosanoyl-glycero-3-phospho-(1′-sn-glycerol); 1-(5Z,8Z,11Z,14Z,17Z-eicosapentaenoyl)-2-(11Z-eicosenoyl)-glycero-3-phospho-(1′-sn-glycerol); 1-(5Z,8Z,11Z,14Z,17Z-eicosapentaenoyl)-2-(11Z,14Z-eicosadienoyl)-glycero-3-phospho-(1′-sn-glycerol); 1-(5Z,8Z,11Z,14Z,17Z-eicosapentaenoyl)-2-(8Z,11Z,14Z-eicosatrienoyl)-glycero-3-phospho-(1′-sn-glycerol); 1-(5Z,8Z,11Z,14Z,17Z-eicosapentaenoyl)-2-(5Z,8Z,11Z,14Z-eicosatetraenoyl)-glycero-3-phospho-(1′-sn-glycerol); 1,2-di-(5Z,8Z,11Z,14Z,17Z-eicosapentaenoyl)-sn-glycero-3-phospho-(1′-sn-glycerol); 1-(5Z,8Z,11Z,14Z,17Z-eicosapentaenoyl)-2-heneicosanoyl-glycero-3-phospho-(1′-sn-glycerol); 1-(5Z,8Z,11Z,14Z,17Z-eicosapentaenoyl)-2-docosanoyl-glycero-3-phospho-(1′-sn-glycerol); 1-(5Z,8Z,11Z,14Z,17Z-eicosapentaenoyl)-2-(11Z-docosenoyl)-glycero-3-phospho-(1′-sn-glycerol); 1-(5Z,8Z,11Z,14Z,17Z-eicosapentaenoyl)-2-(13Z,16Z-docosadienoyl)-glycero-3-phospho-(1′-sn-glycerol); 1-(5Z,8Z,11Z,14Z,17Z-eicosapentaenoyl)-2-(7Z,10Z,13Z,16Z-docosatetraenoyl)-glycero-3-phospho-(1′-sn-glycerol); 1-heneicosanoyl-2-(9Z-tetradecenoyl)-glycero-3-phospho-(1′-sn-glycerol); 1-heneicosanoyl-2-pentadecanoyl-glycero-3-phospho-(1′-sn-glycerol); 1-heneicosanoyl-2-(9Z-pentadecenoyl)-glycero-3-phospho-(1′-sn-glycerol); 1-heneicosanoyl-2-hexadecanoyl-glycero-3-phospho-(1′-sn-glycerol); 1-heneicosanoyl-2-(9Z-hexadecenoyl)-glycero-3-phospho-(1′-sn-glycerol); 1-heneicosanoyl-2-heptadecanoyl-glycero-3-phospho-(1′-sn-glycerol); 1-heneicosanoyl-2-(9Z-heptadecenoyl)-glycero-3-phospho-(1′-sn-glycerol); 1-heneicosanoyl-2-(9Z,12Z-heptadecadienoyl)-glycero-3-phospho-(1′-sn-glycerol); 1-heneicosanoyl-2-(9Z-octadecenoyl)-glycero-3-phospho-(1′-sn-glycerol); 1-heneicosanoyl-2-(9Z,12Z-octadecadienoyl)-glycero-3-phospho-(1′-sn-glycerol); 1-heneicosanoyl-2-(6Z,9Z,12Z-octadecatrienoyl)-glycero-3-phospho-(1′-sn-glycerol); 1-heneicosanoyl-2-(9Z,12Z,15Z-octadecatrienoyl)-glycero-3-phospho-(1′-sn-glycerol); 1-heneicosanoyl-2-(6Z,9Z,12Z,15Z-octadecatetraenoyl)-glycero-3-phospho-(1′-sn-glycerol); 1-heneicosanoyl-2-nonadecanoyl-glycero-3-phospho-(1′-sn-glycerol); 1-heneicosanoyl-2-(9Z-nonadecenoyl)-glycero-3-phospho-(1′-sn-glycerol); 1-heneicosanoyl-2-eicosanoyl-glycero-3-phospho-(1′-sn-glycerol); 1-heneicosanoyl-2-(11Z-eicosenoyl)-glycero-3-phospho-(1′-sn-glycerol); 1-heneicosanoyl-2-(11Z,14Z-eicosadienoyl)-glycero-3-phospho-(1′-sn-glycerol); 1-heneicosanoyl-2-(8Z,11Z,14Z-eicosatrienoyl)-glycero-3-phospho-(1′-sn-glycerol); 1-heneicosanoyl-2-(5Z,8Z,11Z,14Z-eicosatetraenoyl)-glycero-3-phospho-(1′-sn-glycerol); 1-heneicosanoyl-2-(5Z,8Z,11Z,14Z,17Z-eicosapentaenoyl)-glycero-3-phospho-(1′-sn-glycerol); 1-heneicosanoyl-2-docosanoyl-glycero-3-phospho-(1′-sn-glycerol); 1-heneicosanoyl-2-(11Z-docosenoyl)-glycero-3-phospho-(1′-sn-glycerol); 1-heneicosanoyl-2-(13Z,16Z-docosadienoyl)-glycero-3-phospho-(1′-sn-glycerol); 1-heneicosanoyl-2-(7Z,10Z,13Z,16Z-docosatetraenoyl)-glycero-3-phospho-(1′-sn-glycerol); 1-docosanoyl-2-(9Z-tetradecenoyl)-glycero-3-phospho-(1′-sn-glycerol); 1-docosanoyl-2-pentadecanoyl-glycero-3-phospho-(1′-sn-glycerol); 1-docosanoyl-2-(9Z-pentadecenoyl)-glycero-3-phospho-(1′-sn-glycerol); 1-docosanoyl-2-hexadecanoyl-glycero-3-phospho-(1′-sn-glycerol); 1-docosanoyl-2-(9Z-hexadecenoyl)-glycero-3-phospho-(1′-sn-glycerol); 1-docosanoyl-2-heptadecanoyl-glycero-3-phospho-(1′-sn-glycerol); 1-docosanoyl-2-(9Z-heptadecenoyl)-glycero-3-phospho-(1′-sn-glycerol); 1-docosanoyl-2-(9Z,12Z-heptadecadienoyl)-glycero-3-phospho-(1′-sn-glycerol); 1-docosanoyl-2-(9Z,12Z-octadecadienoyl)-glycero-3-phospho-(1′-sn-glycerol); 1-docosanoyl-2-(6Z,9Z,12Z-octadecatrienoyl)-glycero-3-phospho-(1′-sn-glycerol); 1-docosanoyl-2-(9Z,12Z,15Z-octadecatrienoyl)-glycero-3-phospho-(1′-sn-glycerol); 1-docosanoyl-2-(6Z,9Z,12Z,15Z-octadecatetraenoyl)-glycero-3-phospho-(1′-sn-glycerol); 1-docosanoyl-2-nonadecanoyl-glycero-3-phospho-(1′-sn-glycerol); 1-docosanoyl-2-(9Z-nonadecenoyl)-glycero-3-phospho-(1′-sn-glycerol); 1-docosanoyl-2-eicosanoyl-glycero-3-phospho-(1′-sn-glycerol); 1-docosanoyl-2-(11Z,14Z-eicosadienoyl)-glycero-3-phospho-(1′-sn-glycerol); 1-docosanoyl-2-(8Z,11Z,14Z-eicosatrienoyl)-glycero-3-phospho-(1′-sn-glycerol); 1-docosanoyl-2-(5Z,8Z,11Z,14Z-eicosatetraenoyl)-glycero-3-phospho-(1′-sn-glycerol); 1-docosanoyl-2-(5Z,8Z,11Z,14Z,17Z-eicosapentaenoyl)-glycero-3-phospho-(1′-sn-glycerol); 1-docosanoyl-2-heneicosanoyl-glycero-3-phospho-(1′-sn-glycerol); 1-docosanoyl-2-(11Z-docosenoyl)-glycero-3-phospho-(1′-sn-glycerol); 1-docosanoyl-2-(13Z,16Z-docosadienoyl)-glycero-3-phospho-(1′-sn-glycerol); 1-docosanoyl-2-(7Z,10Z,13Z,16Z-docosatetraenoyl)-glycero-3-phospho-(1′-sn-glycerol); 1-(11Z-docosenoyl)-glycero-3-phospho-(1′-sn-glycerol); 1-(11Z-docosenoyl)-2-dodecanoyl-glycero-3-phospho-(1′-sn-glycerol); 1-(11Z-docosenoyl)-2-tridecanoyl-glycero-3-phospho-(1′-sn-glycerol); 1-(11Z-docosenoyl)-2-tetradecanoyl-glycero-3-phospho-(1′-sn-glycerol); 1-(11Z-docosenoyl)-2-(9Z-tetradecenoyl)-glycero-3-phospho-(1′-sn-glycerol); 1-(11Z-docosenoyl)-2-pentadecanoyl-glycero-3-phospho-(1′-sn-glycerol); 1-(11Z-docosenoyl)-2-(9Z-pentadecenoyl)-glycero-3-phospho-(1′-sn-glycerol); 1-(11Z-docosenoyl)-2-hexadecanoyl-glycero-3-phospho-(1′-sn-glycerol); 1-(11Z-docosenoyl)-2-(9Z-hexadecenoyl)-glycero-3-phospho-(1′-sn-glycerol); 1-(11Z-docosenoyl)-2-heptadecanoyl-glycero-3-phospho-(1′-sn-glycerol); 1-(11Z-docosenoyl)-2-(9Z-heptadecenoyl)-glycero-3-phospho-(1′-sn-glycerol); 1-(11Z-docosenoyl)-2-(9Z,12Z-heptadecadienoyl)-glycero-3-phospho-(1′-sn-glycerol); 1-(11Z-docosenoyl)-2-octadecanoyl-glycero-3-phospho-(1′-sn-glycerol); 1-(11Z-docosenoyl)-2-(9Z-octadecenoyl)-glycero-3-phospho-(1′-sn-glycerol); 1-(11Z-docosenoyl)-2-(9Z,12Z-octadecadienoyl)-glycero-3-phospho-(1′-sn-glycerol); 1-(11Z-docosenoyl)-2-(6Z,9Z,12Z-octadecatrienoyl)-glycero-3-phospho-(1′-sn-glycerol); 1-(11Z-docosenoyl)-2-(9Z,12Z,15Z-octadecatrienoyl)-glycero-3-phospho-(1′-sn-glycerol); 1-(11Z-docosenoyl)-2-(6Z,9Z,12Z,15Z-octadecatetraenoyl)-glycero-3-phospho-(1′-sn-glycerol); 1-(11Z-docosenoyl)-2-nonadecanoyl-glycero-3-phospho-(1′-sn-glycerol); 1-(11Z-docosenoyl)-2-(9Z-nonadecenoyl)-glycero-3-phospho-(1′-sn-glycerol); 1-(11Z-docosenoyl)-2-eicosanoyl-glycero-3-phospho-(1′-sn-glycerol); 1-(11Z-docosenoyl)-2-(11Z-eicosenoyl)-glycero-3-phospho-(1′-sn-glycerol); 1-(11Z-docosenoyl)-2-(11Z,14Z-eicosadienoyl)-glycero-3-phospho-(1′-sn-glycerol); 1-(11 Z-docosenoyl)-2-(8Z,11Z,14Z-eicosatrienoyl)-glycero-3-phospho-(1′-sn-glycerol); 1-(11Z-docosenoyl)-2-(5Z,8Z,11Z,14Z-eicosatetraenoyl)-glycero-3-phospho-(1′-sn-glycerol); 1-(11Z-docosenoyl)-2-(5Z,8Z,11Z,14Z,17Z-eicosapentaenoyl)-glycero-3-phospho-(1′-sn-glycerol); 1-(11Z-docosenoyl)-2-heneicosanoyl-glycero-3-phospho-(1′-sn-glycerol); 1-(11Z-docosenoyl)-2-docosanoyl-glycero-3-phospho-(1′-sn-glycerol); 1,2-di-(11Z-docosenoyl)-sn-glycero-3-phospho-(1′-sn-glycerol); 1-(11Z-docosenoyl)-2-(13Z,16Z-docosadienoyl)-glycero-3-phospho-(1′-sn-glycerol); 1-(11Z-docosenoyl)-2-(7Z,10Z,13Z,16Z-docosatetraenoyl)-glycero-3-phospho-(1′-sn-glycerol); 1-(11Z-docosenoyl)-2-(4Z,7Z,10Z,13Z,16Z,19Z-docosahexaenoyl)-glycero-3-phospho-(1′-sn-glycerol); 1-(13Z,16Z-docosadienoyl)-glycero-3-phospho-(1′-sn-glycerol); 1-(13Z,16Z-docosadienoyl)-2-dodecanoyl-glycero-3-phospho-(1′-sn-glycerol); 1-(13Z,16Z-docosadienoyl)-2-tridecanoyl-glycero-3-phospho-(1′-sn-glycerol); 1-(13Z,16Z-docosadienoyl)-2-tetradecanoyl-glycero-3-phospho-(1′-sn-glycerol); 1-(13Z,16Z-docosadienoyl)-2-(9Z-tetradecenoyl)-glycero-3-phospho-(1′-sn-glycerol); 1-(13Z,16Z-docosadienoyl)-2-pentadecanoyl-glycero-3-phospho-(1′-sn-glycerol); 1-(13Z,16Z-docosadienoyl)-2-(9Z-pentadecenoyl)-glycero-3-phospho-(1′-sn-glycerol); 1-(13Z,16Z-docosadienoyl)-2-hexadecanoyl-glycero-3-phospho-(1′-sn-glycerol); 1-(13Z,16Z-docosadienoyl)-2-(9Z-hexadecenoyl)-glycero-3-phospho-(1′-sn-glycerol); 1-(13Z,16Z-docosadienoyl)-2-heptadecanoyl-glycero-3-phospho-(1′-sn-glycerol); 1-(13Z,16Z-docosadienoyl)-2-(9Z-heptadecenoyl)-glycero-3-phospho-(1′-sn-glycerol); 1-(13Z,16Z-docosadienoyl)-2-(9Z,12Z-heptadecadienoyl)-glycero-3-phospho-(1′-sn-glycerol); 1-(13Z,16Z-docosadienoyl)-2-octadecanoyl-glycero-3-phospho-(1′-sn-glycerol); 1-(13Z,16Z-docosadienoyl)-2-(9Z-octadecenoyl)-glycero-3-phospho-(1′-sn-glycerol); 1-(13Z,16Z-docosadienoyl)-2-(9Z,12Z-octadecadienoyl)-glycero-3-phospho-(1′-sn-glycerol); 1-(13Z,16Z-docosadienoyl)-2-(6Z,9Z,12Z-octadecatrienoyl)-glycero-3-phospho-(1′-sn-glycerol); 1-(13Z,16Z-docosadienoyl)-2-(9Z,12Z,15Z-octadecatrienoyl)-glycero-3-phospho-(1′-sn-glycerol); 1-(13Z,16Z-docosadienoyl)-2-(6Z,9Z,12Z,15Z-octadecatetraenoyl)-glycero-3-phospho-(1′-sn-glycerol); 1-(13Z,16Z-docosadienoyl)-2-nonadecanoyl-glycero-3-phospho-(1′-sn-glycerol); 1-(13Z,16Z-docosadienoyl)-2-(9Z-nonadecenoyl)-glycero-3-phospho-(1′-sn-glycerol); 1-(13Z,16Z-docosadienoyl)-2-eicosanoyl-glycero-3-phospho-(1′-sn-glycerol); 1-(13Z,16Z-docosadienoyl)-2-(11Z-eicosenoyl)-glycero-3-phospho-(1′-sn-glycerol); 1-(13Z,16Z-docosadienoyl)-2-(11Z,14Z-eicosadienoyl)-glycero-3-phospho-(1′-sn-glycerol); 1-(13Z,16Z-docosadienoyl)-2-(8Z,11Z,14Z-eicosatrienoyl)-glycero-3-phospho-(1′-sn-glycerol); 1-(13Z,16Z-docosadienoyl)-2-(5Z,8Z,11Z,14Z-eicosatetraenoyl)-glycero-3-phospho-(1′-sn-glycerol); 1-(13Z,16Z-docosadienoyl)-2-(5Z,8Z,11Z,14Z,17Z-eicosapentaenoyl)-glycero-3-phospho-(1′-sn-glycerol); 1-(13Z,16Z-docosadienoyl)-2-heneicosanoyl-glycero-3-phospho-(1′-sn-glycerol); 1-(13Z,16Z-docosadienoyl)-2-docosanoyl-glycero-3-phospho-(1′-sn-glycerol); 1-(13Z,16Z-docosadienoyl)-2-(11Z-docosenoyl)-glycero-3-phospho-(1′-sn-glycerol); 1,2-di-(13Z,16Z-docosadienoyl)-sn-glycero-3-phospho-(1′-sn-glycerol); 1-(13Z,16Z-docosadienoyl)-2-(7Z,10Z,13Z,16Z-docosatetraenoyl)-glycero-3-phospho-(1′-sn-glycerol); 1-(13Z,16Z-docosadienoyl)-2-(4Z,7Z,10Z,13Z,16Z,19Z-docosahexaenoyl)-glycero-3-phospho-(1′-sn-glycerol); 1-(7Z,10Z,13Z,16Z-docosatetraenoyl)-2-dodecanoyl-glycero-3-phospho-(1′-sn-glycerol); 1-(7Z,10Z,13Z,16Z-docosatetraenoyl)-2-tridecanoyl-glycero-3-phospho-(1′-sn-glycerol); 1-(7Z,10Z,13Z,16Z-docosatetraenoyl)-2-tetradecanoyl-glycero-3-phospho-(1′-sn-glycerol); 1-(7Z,10Z,13Z,16Z-docosatetraenoyl)-2-(9Z-tetradecenoyl)-glycero-3-phospho-(1′-sn-glycerol); 1-(7Z,10Z,13Z,16Z-docosatetraenoyl)-2-pentadecanoyl-glycero-3-phospho-(1′-sn-glycerol); 1-(7Z,10Z,13Z,16Z-docosatetraenoyl)-2-(9Z-pentadecenoyl)-glycero-3-phospho-(1′-sn-glycerol); 1-(7Z,10Z,13Z,16Z-docosatetraenoyl)-2-hexadecanoyl-glycero-3-phospho-(1′-sn-glycerol); 1-(7Z,10Z,13Z,16Z-docosatetraenoyl)-2-(9Z-hexadecenoyl)-glycero-3-phospho-(1′-sn-glycerol); 1-(7Z,10Z,13Z,16Z-docosatetraenoyl)-2-heptadecanoyl-glycero-3-phospho-(1′-sn-glycerol); 1-(7Z,10Z,13Z,16Z-docosatetraenoyl)-2-(9Z-heptadecenoyl)-glycero-3-phospho-(1′-sn-glycerol); 1-(7Z,10Z,13Z,16Z-docosatetraenoyl)-2-(9Z,12Z-heptadecadienoyl)-glycero-3-phospho-(1′-sn-glycerol); 1-(7Z,10Z,13Z,16Z-docosatetraenoyl)-2-octadecanoyl-glycero-3-phospho-(1′-sn-glycerol); 1-(7Z,10Z,13Z,16Z-docosatetraenoyl)-2-(9Z-octadecenoyl)-glycero-3-phospho-(1′-sn-glycerol); 1-(7Z,10Z,13Z,16Z-docosatetraenoyl)-2-(9Z,12Z-octadecadienoyl)-glycero-3-phospho-(1′-sn-glycerol); 1-(7Z,10Z,13Z,16Z-docosatetraenoyl)-2-(6Z,9Z,12Z-octadecatrienoyl)-glycero-3-phospho-(1′-sn-glycerol); 1-(7Z,10Z,13Z,16Z-docosatetraenoyl)-2-(9Z,12Z,15Z-octadecatrienoyl)-glycero-3-phospho-(1′-sn-glycerol); 1-(7Z,10Z,13Z,16Z-docosatetraenoyl)-2-(6Z,9Z,12Z,15Z-octadecatetraenoyl)-glycero-3-phospho-(1′-sn-glycerol); 1-(7Z,10Z,13Z,16Z-docosatetraenoyl)-2-nonadecanoyl-glycero-3-phospho-(1′-sn-glycerol); 1-(7Z,10Z,13Z,16Z-docosatetraenoyl)-2-(9Z-nonadecenoyl)-glycero-3-phospho-(1′-sn-glycerol); 1-(7Z,10Z,13Z,16Z-docosatetraenoyl)-2-eicosanoyl-glycero-3-phospho-(1′-sn-glycerol); 1-(7Z,10Z,13Z,16Z-docosatetraenoyl)-2-(11Z-eicosenoyl)-glycero-3-phospho-(1′-sn-glycerol); 1-(7Z,10Z,13Z,16Z-docosatetraenoyl)-2-(11Z,14Z-eicosadienoyl)-glycero-3-phospho-(1′-sn-glycerol); 1-(7Z,10Z,13Z,16Z-docosatetraenoyl)-2-(8Z,11Z,14Z-eicosatrienoyl)-glycero-3-phospho-(1′-sn-glycerol); 1-(7Z,10Z,13Z,16Z-docosatetraenoyl)-2-(5Z,8Z,11Z,14Z-eicosatetraenoyl)-glycero-3-phospho-(1′-sn-glycerol); 1-(7Z,10Z,13Z,16Z-docosatetraenoyl)-2-(5Z,8Z,11Z,14Z,17Z-eicosapentaenoyl)-glycero-3-phospho-(1′-sn-glycerol); 1-(7Z,10Z,13Z,16Z-docosatetraenoyl)-2-heneicosanoyl-glycero-3-phospho-(1′-sn-glycerol); 1-(7Z,10Z,13Z,16Z-docosatetraenoyl)-2-docosanoyl-glycero-3-phospho-(1′-sn-glycerol); 1-(7Z,10Z,13Z,16Z-docosatetraenoyl)-2-(11Z-docosenoyl)-glycero-3-phospho-(1′-sn-glycerol); 1-(7Z,10Z,13Z,16Z-docosatetraenoyl)-2-(13Z,16Z-docosadienoyl)-glycero-3-phospho-(1′-sn-glycerol); 1,2-di-(7Z,10Z,13Z,16Z-docosatetraenoyl)-sn-glycero-3-phospho-(1′-sn-glycerol); 1-(4Z,7Z,10Z,13Z,16Z,19Z-docosahexaenoyl)-2-dodecanoyl-glycero-3-phospho-(1′-sn-glycerol); 1-(4Z,7Z,10Z,13Z,16Z,19Z-docosahexaenoyl)-2-tridecanoyl-glycero-3-phospho-(1′-sn-glycerol); 1-(4Z,7Z,10Z,13Z,16Z,19Z-docosahexaenoyl)-2-tetradecanoyl-glycero-3-phospho-(1′-sn-glycerol); 1-(4Z,7Z,10Z,13Z,16Z,19Z-docosahexaenoyl)-2-(9Z-tetradecenoyl)-glycero-3-phospho-(1′-sn-glycerol); 1-(4Z,7Z,10Z,13Z,16Z,19Z-docosahexaenoyl)-2-pentadecanoyl-glycero-3-phospho-(1′-sn-glycerol); 1-(4Z,7Z,10Z,13Z,16Z,19Z-docosahexaenoyl)-2-(9Z-pentadecenoyl)-glycero-3-phospho-(1′-sn-glycerol); 1-(4Z,7Z,10Z,13Z,16Z,19Z-docosahexaenoyl)-2-(9Z-hexadecenoyl)-glycero-3-phospho-(1′-sn-glycerol); 1-(4Z,7Z,10Z,13Z,16Z,19Z-docosahexaenoyl)-2-heptadecanoyl-glycero-3-phospho-(1′-sn-glycerol); 1-(4Z,7Z,10Z,13Z,16Z,19Z-docosahexaenoyl)-2-(9Z-heptadecenoyl)-glycero-3-phospho-(1′-sn-glycerol); 1-(4Z,7Z,10Z,13Z,16Z,19Z-docosahexaenoyl)-2-(9Z,12Z-heptadecadienoyl)-glycero-3-phospho-(1′-sn-glycerol); 1-(4Z,7Z,10Z,13Z,16Z,19Z-docosahexaenoyl)-2-octadecanoyl-glycero-3-phospho-(1′-sn-glycerol); 1-(4Z,7Z,10Z,13Z,16Z,19Z-docosahexaenoyl)-2-(9Z-octadecenoyl)-glycero-3-phospho-(1′-sn-glycerol); 1-(4Z,7Z,10Z,13Z,16Z,19Z-docosahexaenoyl)-2-(9Z,12Z-octadecadienoyl)-glycero-3-phospho-(1′-sn-glycerol); 1-(4Z,7Z,10Z,13Z,16Z,19Z-docosahexaenoyl)-2-(6Z,9Z,12Z-octadecatrienoyl)-glycero-3-phospho-(1′-sn-glycerol); 1-(4Z,7Z,10Z,13Z,16Z,19Z-docosahexaenoyl)-2-(9Z,12Z,15Z-octadecatrienoyl)-glycero-3-phospho-(1′-sn-glycerol); 1-(4Z,7Z,10Z,13Z,16Z,19Z-docosahexaenoyl)-2-(6Z,9Z,12Z,15Z-octadecatetraenoyl)-glycero-3-phospho-(1′-sn-glycerol); 1-(4Z,7Z,10Z,13Z,16Z,19Z-docosahexaenoyl)-2-nonadecanoyl-glycero-3-phospho-(1′-sn-glycerol); 1-(4Z,7Z,10Z,13Z,16Z,19Z-docosahexaenoyl)-2-(9Z-nonadecenoyl)-glycero-3-phospho-(1′-sn-glycerol); 1-(4Z,7Z,10Z,13Z,16Z,19Z-docosahexaenoyl)-2-eicosanoyl-glycero-3-phospho-(1′-sn-glycerol); 1-(4Z,7Z,10Z,13Z,16Z,19Z-docosahexaenoyl)-2-(11Z-eicosenoyl)-glycero-3-phospho-(1′-sn-glycerol); 1-(4Z,7Z,10Z,13Z,16Z,19Z-docosahexaenoyl)-2-(11Z,14Z-eicosadienoyl)-glycero-3-phospho-(1′-sn-glycerol); 1-(4Z,7Z,10Z,13Z,16Z,19Z-docosahexaenoyl)-2-(8Z,11Z,14Z-eicosatrienoyl)-glycero-3-phospho-(1′-sn-glycerol); 1-(4Z,7Z,10Z,13Z,16Z,19Z-docosahexaenoyl)-2-(5Z,8Z,11 Z,14Z-eicosatetraenoyl)-glycero-3-phospho-(1′-sn-glycerol); 1-(4Z,7Z,10Z,13Z,16Z,19Z-docosahexaenoyl)-2-(5Z,8Z,11Z,14Z,17Z-eicosapentaenoyl)-glycero-3-phospho-(1′-sn-glycerol); 1-(4Z,7Z,10Z,13Z,16Z,19Z-docosahexaenoyl)-2-heneicosanoyl-glycero-3-phospho-(1′-sn-glycerol); 1-(4Z,7Z,10Z,13Z,16Z,19Z-docosahexaenoyl)-2-docosanoyl-glycero-3-phospho-(1′-sn-glycerol); 1-(4Z,7Z,10Z,13Z,16Z,19Z-docosahexaenoyl)-2-(11Z-docosenoyl)-glycero-3-phospho-(1′-sn-glycerol); 1-(4Z,7Z,10Z,13Z,16Z,19Z-docosahexaenoyl)-2-(13Z,16Z-docosadienoyl)-glycero-3-phospho-(1′-sn-glycerol); 1-(4Z,7Z,10Z,13Z,16Z,19Z-docosahexaenoyl)-2-(7Z,10Z,13Z,16Z-docosatetraenoyl)-glycero-3-phospho-(1′-sn-glycerol); 1-hexadecyl-2-dodecanoyl-glycero-3-phospho-(1′-sn-glycerol); 1-hexadecyl-2-tridecanoyl-glycero-3-phospho-(1′-sn-glycerol); 1-hexadecyl-2-tetradecanoyl-glycero-3-phospho-(1′-sn-glycerol); 1-hexadecyl-2-(9Z-tetradecenoyl)-glycero-3-phospho-(1′-sn-glycerol); 1-hexadecyl-2-pentadecanoyl-glycero-3-phospho-(1′-sn-glycerol); 1-hexadecyl-2-(9Z-pentadecenoyl)-glycero-3-phospho-(1′-sn-glycerol); 1-hexadecyl-2-(9Z-hexadecenoyl)-glycero-3-phospho-(1′-sn-glycerol); 1-hexadecyl-2-(9Z-heptadecenoyl)-glycero-3-phospho-(1′-sn-glycerol); 1-hexadecyl-2-(9Z,12Z-heptadecadienoyl)-glycero-3-phospho-(1′-sn-glycerol); 1-hexadecyl-2-(6Z,9Z,12Z,15Z-octadecatetraenoyl)-glycero-3-phospho-(1′-sn-glycerol); 1-hexadecyl-2-(9Z-nonadecenoyl)-glycero-3-phospho-(1′-sn-glycerol); 1-hexadecyl-2-(11Z-eicosenoyl)-glycero-3-phospho-(1′-sn-glycerol); 1-hexadecyl-2-(11Z,14Z-eicosadienoyl)-glycero-3-phospho-(1′-sn-glycerol); 1-hexadecyl-2-heneicosanoyl-glycero-3-phospho-(1′-sn-glycerol); 1-hexadecyl-2-(11Z-docosenoyl)-glycero-3-phospho-(1′-sn-glycerol); 1-hexadecyl-2-(13Z,16Z-docosadienoyl)-glycero-3-phospho-(1′-sn-glycerol); 1-hexadecyl-2-(7Z,10Z,13Z,16Z-docosatetraenoyl)-glycero-3-phospho-(1′-sn-glycerol); 1-octadecyl-2-dodecanoyl-glycero-3-phospho-(1′-sn-glycerol); 1-octadecyl-2-tridecanoyl-glycero-3-phospho-(1′-sn-glycerol); 1-octadecyl-2-tetradecanoyl-glycero-3-phospho-(1′-sn-glycerol); 1-octadecyl-2-(9Z-tetradecenoyl)-glycero-3-phospho-(1′-sn-glycerol); 1-octadecyl-2-pentadecanoyl-glycero-3-phospho-(1′-sn-glycerol); 1-octadecyl-2-(9Z-pentadecenoyl)-glycero-3-phospho-(1′-sn-glycerol); 1-octadecyl-2-heptadecanoyl-glycero-3-phospho-(1′-sn-glycerol); 1-octadecyl-2-(9Z-heptadecenoyl)-glycero-3-phospho-(1′-sn-glycerol); 1-octadecyl-2-(9Z,12Z-heptadecadienoyl)-glycero-3-phospho-(1′-sn-glycerol); 1-octadecyl-2-(9Z-octadecenoyl)-glycero-3-phospho-(1′-sn-glycerol); 1-octadecyl-2-(9Z,12Z-octadecadienoyl)-glycero-3-phospho-(1′-sn-glycerol); 1-octadecyl-2-(6Z,9Z,12Z-octadecatrienoyl)-glycero-3-phospho-(1′-sn-glycerol); 1-octadecyl-2-(9Z,12Z,15Z-octadecatrienoyl)-glycero-3-phospho-(1′-sn-glycerol); 1-octadecyl-2-(6Z,9Z,12Z,15Z-octadecatetraenoyl)-glycero-3-phospho-(1′-sn-glycerol); 1-octadecyl-2-nonadecanoyl-glycero-3-phospho-(1′-sn-glycerol); 1-octadecyl-2-(9Z-nonadecenoyl)-glycero-3-phospho-(1′-sn-glycerol); 1-octadecyl-2-(11Z-eicosenoyl)-glycero-3-phospho-(1′-sn-glycerol); 1-octadecyl-2-(11Z,14Z-eicosadienoyl)-glycero-3-phospho-(1′-sn-glycerol); 1-octadecyl-2-(8Z,11Z,14Z-eicosatrienoyl)-glycero-3-phospho-(1′-sn-glycerol); 1-octadecyl-2-heneicosanoyl-glycero-3-phospho-(1′-sn-glycerol); 1-octadecyl-2-(11Z-docosenoyl)-glycero-3-phospho-(1′-sn-glycerol); 1-octadecyl-2-(13Z,16Z-docosadienoyl)-glycero-3-phospho-(1′-sn-glycerol); 1-octadecyl-2-(7Z,10Z,13Z,16Z-docosatetraenoyl)-glycero-3-phospho-(1′-sn-glycerol); 1-eicosyl-glycero-3-phospho-(1′-sn-glycerol); 1-eicosyl-2-dodecanoyl-glycero-3-phospho-(1′-sn-glycerol); 1-eicosyl-2-tridecanoyl-glycero-3-phospho-(1′-sn-glycerol); 1-eicosyl-2-tetradecanoyl-glycero-3-phospho-(1′-sn-glycerol); 1-eicosyl-2-(9Z-tetradecenoyl)-glycero-3-phospho-(1′-sn-glycerol); 1-eicosyl-2-pentadecanoyl-glycero-3-phospho-(1′-sn-glycerol); 1-eicosyl-2-(9Z-pentadecenoyl)-glycero-3-phospho-(1′-sn-glycerol); 1-eicosyl-2-(9Z-hexadecenoyl)-glycero-3-phospho-(1′-sn-glycerol); 1-eicosyl-2-heptadecanoyl-glycero-3-phospho-(1′-sn-glycerol); 1-eicosyl-2-(9Z-heptadecenoyl)-glycero-3-phospho-(1′-sn-glycerol); 1-eicosyl-2-(9Z,12Z-heptadecadienoyl)-glycero-3-phospho-(1′-sn-glycerol); 1-eicosyl-2-octadecanoyl-glycero-3-phospho-(1′-sn-glycerol); 1-eicosyl-2-(9Z-octadecenoyl)-glycero-3-phospho-(1′-sn-glycerol); 1-eicosyl-2-(9Z,12Z-octadecadienoyl)-glycero-3-phospho-(1′-sn-glycerol); 1-eicosyl-2-(6Z,9Z,12Z-octadecatrienoyl)-glycero-3-phospho-(1′-sn-glycerol); 1-eicosyl-2-(9Z,12Z,15Z-octadecatrienoyl)-glycero-3-phospho-(1′-sn-glycerol); 1-eicosyl-2-(6Z,9Z,12Z,15Z-octadecatetraenoyl)-glycero-3-phospho-(1′-sn-glycerol); 1-eicosyl-2-nonadecanoyl-glycero-3-phospho-(1′-sn-glycerol); 1-eicosyl-2-(9Z-nonadecenoyl)-glycero-3-phospho-(1′-sn-glycerol); 1-eicosyl-2-eicosanoyl-glycero-3-phospho-(1′-sn-glycerol); 1-eicosyl-2-(11Z-eicosenoyl)-glycero-3-phospho-(1′-sn-glycerol); 1-eicosyl-2-(11Z,14Z-eicosadienoyl)-glycero-3-phospho-(1′-sn-glycerol); 1-eicosyl-2-(8Z,11Z,14Z-eicosatrienoyl)-glycero-3-phospho-(1′-sn-glycerol); 1-eicosyl-2-(5Z,8Z,11Z,14Z-eicosatetraenoyl)-glycero-3-phospho-(1′-sn-glycerol); 1-eicosyl-2-(5Z,8Z,11Z,14Z,17Z-eicosapentaenoyl)-glycero-3-phospho-(1′-sn-glycerol); 1-eicosyl-2-heneicosanoyl-glycero-3-phospho-(1′-sn-glycerol); 1-eicosyl-2-(11Z-docosenoyl)-glycero-3-phospho-(1′-sn-glycerol); 1-eicosyl-2-(13Z,16Z-docosadienoyl)-glycero-3-phospho-(1′-sn-glycerol); 1-eicosyl-2-(7Z,10Z,13Z,16Z-docosatetraenoyl)-glycero-3-phospho-(1′-sn-glycerol); 1-(1Z-hexadecenyl)-2-dodecanoyl-glycero-3-phospho-(1′-sn-glycerol); 1-(1Z-hexadecenyl)-2-tridecanoyl-glycero-3-phospho-(1′-sn-glycerol); 1-(1Z-hexadecenyl)-2-tetradecanoyl-glycero-3-phospho-(1′-sn-glycerol); 1-(1Z-hexadecenyl)-2-(9Z-tetradecenoyl)-glycero-3-phospho-(1′-sn-glycerol); 1-(1Z-hexadecenyl)-2-pentadecanoyl-glycero-3-phospho-(1′-sn-glycerol); 1-(1Z-hexadecenyl)-2-(9Z-pentadecenoyl)-glycero-3-phospho-(1′-sn-glycerol); 1-(1Z-hexadecenyl)-2-hexadecanoyl-glycero-3-phospho-(1′-sn-glycerol); 1-(1Z-hexadecenyl)-2-(9Z-hexadecenoyl)-glycero-3-phospho-(1′-sn-glycerol); 1-(1Z-hexadecenyl)-2-heptadecanoyl-glycero-3-phospho-(1′-sn-glycerol); 1-(1Z-hexadecenyl)-2-(9Z-heptadecenoyl)-glycero-3-phospho-(1′-sn-glycerol); 1-(1Z-hexadecenyl)-2-(9Z,12Z-heptadecadienoyl)-glycero-3-phospho-(1′-sn-glycerol); 1-(1Z-hexadecenyl)-2-octadecanoyl-glycero-3-phospho-(1′-sn-glycerol); 1-(1Z-hexadecenyl)-2-(6Z,9Z,12Z-octadecatrienoyl)-glycero-3-phospho-(1′-sn-glycerol); 1-(1Z-hexadecenyl)-2-(9Z,12Z,15Z-octadecatrienoyl)-glycero-3-phospho-(1′-sn-glycerol); 1-(1Z-hexadecenyl)-2-(6Z,9Z,12Z,15Z-octadecatetraenoyl)-glycero-3-phospho-(1′-sn-glycerol); 1-(1Z-hexadecenyl)-2-nonadecanoyl-glycero-3-phospho-(1′-sn-glycerol); 1-(1Z-hexadecenyl)-2-(9Z-nonadecenoyl)-glycero-3-phospho-(1′-sn-glycerol); 1-(1Z-hexadecenyl)-2-eicosanoyl-glycero-3-phospho-(1′-sn-glycerol); 1-(1Z-hexadecenyl)-2-(11Z-eicosenoyl)-glycero-3-phospho-(1′-sn-glycerol); 1-(1Z-hexadecenyl)-2-(11Z,14Z-eicosadienoyl)-glycero-3-phospho-(1′-sn-glycerol); 1-(1Z-hexadecenyl)-2-(8Z,11Z,14Z-eicosatrienoyl)-glycero-3-phospho-(1′-sn-glycerol); 1-(1Z-hexadecenyl)-2-(5Z,8Z,11Z,14Z,17Z-eicosapentaenoyl)-glycero-3-phospho-(1′-sn-glycerol); 1-(1Z-hexadecenyl)-2-heneicosanoyl-glycero-3-phospho-(1′-sn-glycerol); 1-(1Z-hexadecenyl)-2-docosanoyl-glycero-3-phospho-(1′-sn-glycerol); 1-(1Z-hexadecenyl)-2-(11Z-docosenoyl)-glycero-3-phospho-(1′-sn-glycerol); 1-(1Z-hexadecenyl)-2-(13Z,16Z-docosadienoyl)-glycero-3-phospho-(1′-sn-glycerol); 1-(1Z-hexadecenyl)-2-(7Z,10Z,13Z,16Z-docosatetraenoyl)-glycero-3-phospho-(1′-sn-glycerol); 1-(1Z-octadecenyl)-2-dodecanoyl-glycero-3-phospho-(1′-sn-glycerol); 1-(1Z-octadecenyl)-2-tridecanoyl-glycero-3-phospho-(1′-sn-glycerol); 1-(1Z-octadecenyl)-2-tetradecanoyl-glycero-3-phospho-(1′-sn-glycerol); 1-(1Z-octadecenyl)-2-(9Z-tetradecenoyl)-glycero-3-phospho-(1′-sn-glycerol); 1-(1Z-octadecenyl)-2-pentadecanoyl-glycero-3-phospho-(1′-sn-glycerol); 1-(1Z-octadecenyl)-2-(9Z-pentadecenoyl)-glycero-3-phospho-(1′-sn-glycerol); 1-(1Z-octadecenyl)-2-hexadecanoyl-glycero-3-phospho-(1′-sn-glycerol); 1-(1Z-octadecenyl)-2-(9Z-hexadecenoyl)-glycero-3-phospho-(1′-sn-glycerol); 1-(1Z-octadecenyl)-2-heptadecanoyl-glycero-3-phospho-(1′-sn-glycerol); 1-(1Z-octadecenyl)-2-(9Z-heptadecenoyl)-glycero-3-phospho-(1′-sn-glycerol); 1-(1Z-octadecenyl)-2-(9Z,12Z-heptadecadienoyl)-glycero-3-phospho-(1′-sn-glycerol); 1-(1Z-octadecenyl)-2-octadecanoyl-glycero-3-phospho-(1′-sn-glycerol); 1-(1Z-octadecenyl)-2-(9Z,12Z-octadecadienoyl)-glycero-3-phospho-(1′-sn-glycerol); 1-(1Z-octadecenyl)-2-(6Z,9Z,12Z-octadecatrienoyl)-glycero-3-phospho-(1′-sn-glycerol); 1-(1Z-octadecenyl)-2-(9Z,12Z,15Z-octadecatrienoyl)-glycero-3-phospho-(1′-sn-glycerol); 1-(1Z-octadecenyl)-2-(6Z,9Z,12Z,15Z-octadecatetraenoyl)-glycero-3-phospho-(1′-sn-glycerol); 1-(1Z-octadecenyl)-2-nonadecanoyl-glycero-3-phospho-(1′-sn-glycerol); 1-(1Z-octadecenyl)-2-(9Z-nonadecenoyl)-glycero-3-phospho-(1′-sn-glycerol); 1-(1Z-octadecenyl)-2-eicosanoyl-glycero-3-phospho-(1′-sn-glycerol); 1-(1Z-octadecenyl)-2-(11Z-eicosenoyl)-glycero-3-phospho-(1′-sn-glycerol); 1-(1Z-octadecenyl)-2-(11Z,14Z-eicosadienoyl)-glycero-3-phospho-(1′-sn-glycerol); 1-(1Z-octadecenyl)-2-(8Z,11Z,14Z-eicosatrienoyl)-glycero-3-phospho-(1′-sn-glycerol); 1-(1Z-octadecenyl)-2-(5Z,8Z,11Z,14Z,17Z-eicosapentaenoyl)-glycero-3-phospho-(1′-sn-glycerol); 1-(1Z-octadecenyl)-2-heneicosanoyl-glycero-3-phospho-(1′-sn-glycerol); 1-(1Z-octadecenyl)-2-docosanoyl-glycero-3-phospho-(1′-sn-glycerol); 1-(1Z-octadecenyl)-2-(11Z-docosenoyl)-glycero-3-phospho-(1′-sn-glycerol); 1-(1Z-octadecenyl)-2-(13Z,16Z-docosadienoyl)-glycero-3-phospho-(1′-sn-glycerol); 1-(1Z-octadecenyl)-2-(7Z,10Z,13Z,16Z-docosatetraenoyl)-glycero-3-phospho-(1′-sn-glycerol); 1-(1Z-eicosenyl)-glycero-3-phospho-(1′-sn-glycerol); 1-(1Z-eicosenyl)-2-dodecanoyl-glycero-3-phospho-(1′-sn-glycerol); 1-(1Z-eicosenyl)-2-tridecanoyl-glycero-3-phospho-(1′-sn-glycerol); 1-(1Z-eicosenyl)-2-tetradecanoyl-glycero-3-phospho-(1′-sn-glycerol); 1-(1Z-eicosenyl)-2-(9Z-tetradecenoyl)-glycero-3-phospho-(1′-sn-glycerol); 1-(1Z-eicosenyl)-2-pentadecanoyl-glycero-3-phospho-(1′-sn-glycerol); 1-(1Z-eicosenyl)-2-(9Z-pentadecenoyl)-glycero-3-phospho-(1′-sn-glycerol); 1-(1Z-eicosenyl)-2-hexadecanoyl-glycero-3-phospho-(1′-sn-glycerol); 1-(1Z-eicosenyl)-2-(9Z-hexadecenoyl)-glycero-3-phospho-(1′-sn-glycerol); 1-(1Z-eicosenyl)-2-heptadecanoyl-glycero-3-phospho-(1′-sn-glycerol); 1-(1Z-eicosenyl)-2-(9Z-heptadecenoyl)-glycero-3-phospho-(1′-sn-glycerol); 1-(1Z-eicosenyl)-2-(9Z,12Z-heptadecadienoyl)-glycero-3-phospho-(1′-sn-glycerol); 1-(1Z-eicosenyl)-2-octadecanoyl-glycero-3-phospho-(1′-sn-glycerol); 1-(1Z-eicosenyl)-2-(9Z-octadecenoyl)-glycero-3-phospho-(1′-sn-glycerol); 1-(1Z-eicosenyl)-2-(9Z,12Z-octadecadienoyl)-glycero-3-phospho-(1′-sn-glycerol); 1-(1Z-eicosenyl)-2-(6Z,9Z,12Z-octadecatrienoyl)-glycero-3-phospho-(1′-sn-glycerol); 1-(1Z-eicosenyl)-2-(9Z,12Z,15Z-octadecatrienoyl)-glycero-3-phospho-(1′-sn-glycerol); 1-(1Z-eicosenyl)-2-(6Z,9Z,12Z,15Z-octadecatetraenoyl)-glycero-3-phospho-(1′-sn-glycerol); 1-(1Z-eicosenyl)-2-nonadecanoyl-glycero-3-phospho-(1′-sn-glycerol); 1-(1Z-eicosenyl)-2-(9Z-nonadecenoyl)-glycero-3-phospho-(1′-sn-glycerol); 1-(1Z-eicosenyl)-2-eicosanoyl-glycero-3-phospho-(1′-sn-glycerol); 1-(1Z-eicosenyl)-2-(11Z-eicosenoyl)-glycero-3-phospho-(1′-sn-glycerol); 1-(1Z-eicosenyl)-2-(11Z,14Z-eicosadienoyl)-glycero-3-phospho-(1′-sn-glycerol); 1-(1Z-eicosenyl)-2-(8Z,11Z,14Z-eicosatrienoyl)-glycero-3-phospho-(1′-sn-glycerol); 1-(1Z-eicosenyl)-2-(5Z,8Z,11Z,14Z-eicosatetraenoyl)-glycero-3-phospho-(1′-sn-glycerol); 1-(1Z-eicosenyl)-2-(5Z,8Z,11Z,14Z,17Z-eicosapentaenoyl)-glycero-3-phospho-(1′-sn-glycerol); 1-(1Z-eicosenyl)-2-heneicosanoyl-glycero-3-phospho-(1′-sn-glycerol); 1-(1Z-eicosenyl)-2-docosanoyl-glycero-3-phospho-(1′-sn-glycerol); 1-(1Z-eicosenyl)-2-(11Z-docosenoyl)-glycero-3-phospho-(1′-sn-glycerol); 1-(1Z-eicosenyl)-2-(13Z,16Z-docosadienoyl)-glycero-3-phospho-(1′-sn-glycerol); 1-(1Z-eicosenyl)-2-(7Z,10Z,13Z,16Z-docosatetraenoyl)-glycero-3-phospho-(1′-sn-glycerol); 1-(1Z-hexadecenyl)-2-(5Z,8Z,11Z,14Z-eicosatetraenoyl)-glycero-3-phospho-(1′-sn-glycerol); 1-(1Z-hexadecenyl)-2-(9Z,12Z-octadecadienoyl)-glycero-3-phospho-(1′-sn-glycerol); 1-(1Z-hexadecenyl)-2-(9Z-octadecenoyl)-glycero-3-phospho-(1′-sn-glycerol); 1-eicosyl-2-docosanoyl-glycero-3-phospho-(1′-sn-glycerol); 1-eicosyl-2-hexadecanoyl-glycero-3-phospho-(1′-sn-glycerol); 1-octadecyl-2-(5Z,8Z,11Z,14Z-eicosatetraenoyl)-glycero-3-phospho-(1′-sn-glycerol); 1-octadecyl-2-(9Z-hexadecenoyl)-glycero-3-phospho-(1′-sn-glycerol); 1-octadecyl-2-hexadecanoyl-glycero-3-phospho-(1′-sn-glycerol); 1-hexadecyl-2-(5Z,8Z,11Z,14Z,17Z-eicosapentaenoyl)-glycero-3-phospho-(1′-sn-glycerol); 1-hexadecyl-2-(8Z,11Z,14Z-eicosatrienoyl)-glycero-3-phospho-(1′-sn-glycerol); 1-hexadecyl-2-nonadecanoyl-glycero-3-phospho-(1′-sn-glycerol); 1-hexadecyl-2-(9Z,12Z,15Z-octadecatrienoyl)-glycero-3-phospho-(1′-sn-glycerol); 1-hexadecyl-2-(6Z,9Z,12Z-octadecatrienoyl)-glycero-3-phospho-(1′-sn-glycerol); 1-hexadecyl-2-(9Z,12Z-octadecadienoyl)-glycero-3-phospho-(1′-sn-glycerol); 1-hexadecyl-2-octadecanoyl-glycero-3-phospho-(1′-sn-glycerol); 1-hexadecyl-2-heptadecanoyl-glycero-3-phospho-(1′-sn-glycerol); 1-hexadecyl-2-hexadecanoyl-glycero-3-phospho-(1′-sn-glycerol); 1-(4Z,7Z,10Z,13Z,16Z,19Z-docosahexaenoyl)-2-hexadecanoyl-glycero-3-phospho-(1′-sn-glycerol); 1-docosanoyl-2-octadecanoyl-glycero-3-phospho-(1′-sn-glycerol); 1-docosanoyl-2-tetradecanoyl-glycero-3-phospho-(1′-sn-glycerol); 1-docosanoyl-2-tridecanoyl-glycero-3-phospho-(1′-sn-glycerol); 1-docosanoyl-2-dodecanoyl-glycero-3-phospho-(1′-sn-glycerol); 1-docosanoyl-glycero-3-phospho-(1′-sn-glycerol); 1,2-diheneicosanoyl-sn-glycero-3-phospho-(1′-sn-glycerol); 1-heneicosanoyl-2-octadecanoyl-glycero-3-phospho-(1′-sn-glycerol); 1-heneicosanoyl-2-tetradecanoyl-glycero-3-phospho-(1′-sn-glycerol); 1-heneicosanoyl-2-tridecanoyl-glycero-3-phospho-(1′-sn-glycerol); 1-heneicosanoyl-2-dodecanoyl-glycero-3-phospho-(1′-sn-glycerol); 1-heneicosanoyl-glycero-3-phospho-(1′-sn-glycerol); 1-(5Z,8Z,11Z,14Z,17Z-eicosapentaenoyl)-2-(4Z,7Z,10Z,13Z,16Z,19Z-docosahexaenoyl)-glycero-3-phospho-(1′-sn-glycerol); 1-(5Z,8Z,11Z,14Z,17Z-eicosapentaenoyl)-2-(9Z-octadecenoyl)-glycero-3-phospho-(1′-sn-glycerol); 1-(5Z,8Z,11Z,14Z,17Z-eicosapentaenoyl)-glycero-3-phospho-(1′-sn-glycerol); 1,2-di-(5Z,8Z,11Z,14Z-eicosatetraenoyl)-sn-glycero-3-phospho-(1′-sn-glycerol); 1-(5Z,8Z,11Z,14Z-eicosatetraenoyl)-2-hexadecanoyl-glycero-3-phospho-(1′-sn-glycerol); 1,2-di-(11Z-eicosenoyl)-sn-glycero-3-phospho-(1′-sn-glycerol); 1-eicosanoyl-2-(4Z,7Z,10Z,13Z,16Z,19Z-docosahexaenoyl)-glycero-3-phospho-(1′-sn-glycerol); 1-eicosanoyl-2-(5Z,8Z,11Z,14Z-eicosatetraenoyl)-glycero-3-phospho-(1′-sn-glycerol); 1,2-dieicosanoyl-sn-glycero-3-phospho-(1′-sn-glycerol); 1-eicosanoyl-2-hexadecanoyl-glycero-3-phospho-(1′-sn-glycerol); 1-eicosanoyl-2-tetradecanoyl-glycero-3-phospho-(1′-sn-glycerol); 1-eicosanoyl-2-tridecanoyl-glycero-3-phospho-(1′-sn-glycerol); 1-eicosanoyl-2-dodecanoyl-glycero-3-phospho-(1′-sn-glycerol); 1,2-dinonadecanoyl-sn-glycero-3-phospho-(1′-sn-glycerol); 1-nonadecanoyl-2-octadecanoyl-glycero-3-phospho-(1′-sn-glycerol); 1-nonadecanoyl-2-pentadecanoyl-glycero-3-phospho-(1′-sn-glycerol); 1-nonadecanoyl-2-tetradecanoyl-glycero-3-phospho-(1′-sn-glycerol); 1-nonadecanoyl-2-tridecanoyl-glycero-3-phospho-(1′-sn-glycerol); 1-nonadecanoyl-2-dodecanoyl-glycero-3-phospho-(1′-sn-glycerol); 1-nonadecanoyl-glycero-3-phospho-(1′-sn-glycerol); 1-(9Z,12Z,15Z-octadecatrienoyl)-2-(9Z-octadecenoyl)-glycero-3-phospho-(1′-sn-glycerol); 1-(9Z,12Z,15Z-octadecatrienoyl)-glycero-3-phospho-(1′-sn-glycerol); 1-(9Z,12Z-octadecadienoyl)-2-(5Z,8Z,11Z,14Z-eicosatetraenoyl)-glycero-3-phospho-(1′-sn-glycerol); 1-(9Z,12Z-octadecadienoyl)-2-octadecanoyl-glycero-3-phospho-(1′-sn-glycerol); 1-(9Z,12Z-octadecadienoyl)-2-hexadecanoyl-glycero-3-phospho-(1′-sn-glycerol); 1-(9Z-octadecenoyl)-2-(5Z,8Z,11Z,14Z,17Z-eicosapentaenoyl)-glycero-3-phospho-(1′-sn-glycerol); 1-(9Z-octadecenoyl)-2-(5Z,8Z,11Z,14Z-eicosatetraenoyl)-glycero-3-phospho-(1′-sn-glycerol); 1-(9Z-octadecenoyl)-2-(9Z,12Z,15Z-octadecatrienoyl)-glycero-3-phospho-(1′-sn-glycerol); 1-(9Z-octadecenoyl)-2-(9Z-hexadecenoyl)-glycero-3-phospho-(1′-sn-glycerol); 1-(9Z-octadecenoyl)-2-tetradecanoyl-glycero-3-phospho-(1′-sn-glycerol); 1-octadecanoyl-2-(7Z,10Z,13Z,16Z-docosatetraenoyl)-glycero-3-phospho-(1′-sn-glycerol); 1-octadecanoyl-2-(5Z,8Z,11Z,14Z,17Z-eicosapentaenoyl)-glycero-3-phospho-(1′-sn-glycerol); 1-octadecanoyl-2-(8Z,11Z,14Z-eicosatrienoyl)-glycero-3-phospho-(1′-sn-glycerol); 1-octadecanoyl-2-(6Z,9Z,12Z,15Z-octadecatetraenoyl)-glycero-3-phospho-(1′-sn-glycerol); 1-octadecanoyl-2-(9Z-hexadecenoyl)-glycero-3-phospho-(1′-sn-glycerol); 1-octadecanoyl-2-hexadecanoyl-glycero-3-phospho-(1′-sn-glycerol); 1-octadecanoyl-2-dodecanoyl-glycero-3-phospho-(1′-sn-glycerol); 1,2-di-(9Z,12Z-heptadecadienoyl)-sn-glycero-3-phospho-(1′-sn-glycerol); 1,2-di-(9Z-heptadecenoyl)-sn-glycero-3-phospho-(1′-sn-glycerol); 1-heptadecanoyl-2-(4Z,7Z,10Z,13Z,16Z,19Z-docosahexaenoyl)-glycero-3-phospho-(1′-sn-glycerol); 1-heptadecanoyl-2-(9Z-octadecenoyl)-glycero-3-phospho-(1′-sn-glycerol); 1-heptadecanoyl-2-octadecanoyl-glycero-3-phospho-(1′-sn-glycerol); 1,2-diheptadecanoyl-sn-glycero-3-phospho-(1′-sn-glycerol); 1-heptadecanoyl-2-pentadecanoyl-glycero-3-phospho-(1′-sn-glycerol); 1-heptadecanoyl-2-tridecanoyl-glycero-3-phospho-(1′-sn-glycerol); 1-heptadecanoyl-glycero-3-phospho-(1′-sn-glycerol); 1-(9Z-hexadecenoyl)-2-(4Z,7Z,10Z,13Z,16Z,19Z-docosahexaenoyl)-glycero-3-phospho-(1′-sn-glycerol); 1-(9Z-hexadecenoyl)-2-(5Z,8Z,11Z,14Z-eicosatetraenoyl)-glycero-3-phospho-(1′-sn-glycerol); 1-(9Z-hexadecenoyl)-2-(6Z,9Z,12Z,15Z-octadecatetraenoyl)-glycero-3-phospho-(1′-sn-glycerol); 1-(9Z-hexadecenoyl)-2-(9Z,12Z-octadecadienoyl)-glycero-3-phospho-(1′-sn-glycerol); 1-(9Z-hexadecenoyl)-2-(9Z-octadecenoyl)-glycero-3-phospho-(1′-sn-glycerol); 1-hexadecanoyl-2-(5Z,8Z,11Z,14Z,17Z-eicosapentaenoyl)-glycero-3-phospho-(1′-sn-glycerol); 1-hexadecanoyl-2-(8Z,11Z,14Z-eicosatrienoyl)-glycero-3-phospho-(1′-sn-glycerol); 1-hexadecanoyl-2-(6Z,9Z,12Z,15Z-octadecatetraenoyl)-glycero-3-phospho-(1′-sn-glycerol); 1-hexadecanoyl-2-(6Z,9Z,12Z-octadecatrienoyl)-glycero-3-phospho-(1′-sn-glycerol); 1-hexadecanoyl-2-octadecanoyl-glycero-3-phospho-(1′-sn-glycerol); 1-hexadecanoyl-2-(9Z-heptadecenoyl)-glycero-3-phospho-(1′-sn-glycerol); 1-hexadecanoyl-2-heptadecanoyl-glycero-3-phospho-(1′-sn-glycerol); 1-hexadecanoyl-2-(9Z-hexadecenoyl)-glycero-3-phospho-(1′-sn-glycerol); 1-hexadecanoyl-2-pentadecanoyl-glycero-3-phospho-(1′-sn-glycerol); 1-pentadecanoyl-2-heneicosanoyl-glycero-3-phospho-(1′-sn-glycerol); 1-pentadecanoyl-2-nonadecanoyl-glycero-3-phospho-(1′-sn-glycerol); 1-pentadecanoyl-2-(9Z,12Z-octadecadienoyl)-glycero-3-phospho-(1′-sn-glycerol); 1-pentadecanoyl-2-octadecanoyl-glycero-3-phospho-(1′-sn-glycerol); 1-pentadecanoyl-2-heptadecanoyl-glycero-3-phospho-(1′-sn-glycerol); 1-pentadecanoyl-2-(9Z-hexadecenoyl)-glycero-3-phospho-(1′-sn-glycerol); 1-pentadecanoyl-2-hexadecanoyl-glycero-3-phospho-(1′-sn-glycerol); 1-pentadecanoyl-2-tridecanoyl-glycero-3-phospho-(1′-sn-glycerol); 1-pentadecanoyl-glycero-3-phospho-(1′-sn-glycerol); 1,2-di-(9Z-tetradecenoyl)-sn-glycero-3-phospho-(1′-sn-glycerol); 1-(9Z-tetradecenoyl)-glycero-3-phospho-(1′-sn-glycerol); 1-tetradecanoyl-2-(4Z,7Z,10Z,13Z,16Z,19Z-docosahexaenoyl)-glycero-3-phospho-(1′-sn-glycerol); 1-tetradecanoyl-2-docosanoyl-glycero-3-phospho-(1′-sn-glycerol); 1-tetradecanoyl-2-(5Z,8Z,11Z,14Z,17Z-eicosapentaenoyl)-glycero-3-phospho-(1′-sn-glycerol); 1-tetradecanoyl-2-(5Z,8Z,11Z,14Z-eicosatetraenoyl)-glycero-3-phospho-(1′-sn-glycerol); 1-tetradecanoyl-2-eicosanoyl-glycero-3-phospho-(1′-sn-glycerol); 1-tetradecanoyl-2-(6Z,9Z,12Z,15Z-octadecatetraenoyl)-glycero-3-phospho-(1′-sn-glycerol); 1-tetradecanoyl-2-(9Z,12Z,15Z-octadecatrienoyl)-glycero-3-phospho-(1′-sn-glycerol); 1-tetradecanoyl-2-(9Z,12Z-octadecadienoyl)-glycero-3-phospho-(1′-sn-glycerol); 1-tetradecanoyl-2-octadecanoyl-glycero-3-phospho-(1′-sn-glycerol); 1-tetradecanoyl-2-(9Z-hexadecenoyl)-glycero-3-phospho-(1′-sn-glycerol); 1-tetradecanoyl-2-pentadecanoyl-glycero-3-phospho-(1′-sn-glycerol); 1-tetradecanoyl-2-dodecanoyl-glycero-3-phospho-(1′-sn-glycerol); 1-tridecanoyl-2-heneicosanoyl-glycero-3-phospho-(1′-sn-glycerol); 1-tridecanoyl-2-eicosanoyl-glycero-3-phospho-(1′-sn-glycerol); 1-tridecanoyl-2-nonadecanoyl-glycero-3-phospho-(1′-sn-glycerol); 1-tridecanoyl-2-heptadecanoyl-glycero-3-phospho-(1′-sn-glycerol); 1-tridecanoyl-2-pentadecanoyl-glycero-3-phospho-(1′-sn-glycerol); 1-dodecanoyl-2-(4Z,7Z,10Z,13Z,16Z,19Z-docosahexaenoyl)-glycero-3-phospho-(1′-sn-glycerol); 1-dodecanoyl-2-docosanoyl-glycero-3-phospho-(1′-sn-glycerol); 1-dodecanoyl-2-eicosanoyl-glycero-3-phospho-(1′-sn-glycerol); 1-dodecanoyl-2-(9Z-octadecenoyl)-glycero-3-phospho-(1′-sn-glycerol); 1-dodecanoyl-2-octadecanoyl-glycero-3-phospho-(1′-sn-glycerol); 1-dodecanoyl-2-hexadecanoyl-glycero-3-phospho-(1′-sn-glycerol); 1-(1Z-octadecenyl)-2-(4Z,7Z,10Z,13Z,16Z,19Z-docosahexaenoyl)-glycero-3-phospho-(1′-sn-glycerol); 1-(1Z-octadecenyl)-2-(5Z,8Z,11Z,14Z-eicosatetraenoyl)-glycero-3-phospho-(1′-sn-glycerol); 1-(1Z-octadecenyl)-2-(9Z-octadecenoyl)-glycero-3-phospho-(1′-sn-glycerol); 1-(1Z-octadecenyl)-glycero-3-phospho-(1′-sn-glycerol); 1-(1Z-hexadecenyl)-2-(4Z,7Z,10Z,13Z,16Z,19Z-docosahexaenoyl)-glycero-3-phospho-(1′-sn-glycerol); 1-(1Z-hexadecenyl)-glycero-3-phospho-(1′-sn-glycerol); 1-octadecyl-2-(4Z,7Z,10Z,13Z,16Z,19Z-docosahexaenoyl)-glycero-3-phospho-(1′-sn-glycerol); 1-octadecyl-2-docosanoyl-glycero-3-phospho-(1′-sn-glycerol); 1-octadecyl-2-(5Z,8Z,11Z,14Z,17Z-eicosapentaenoyl)-glycero-3-phospho-(1′-sn-glycerol); 1-octadecyl-2-eicosanoyl-glycero-3-phospho-(1′-sn-glycerol); 1-octadecyl-2-octadecanoyl-glycero-3-phospho-(1′-sn-glycerol); 1-octadecyl-glycero-3-phospho-(1′-sn-glycerol); 1-hexadecyl-2-(4Z,7Z,10Z,13Z,16Z,19Z-docosahexaenoyl)-glycero-3-phospho-(1′-sn-glycerol); 1-hexadecyl-2-docosanoyl-glycero-3-phospho-(1′-sn-glycerol); 1-hexadecyl-2-(5Z,8Z,11Z,14Z-eicosatetraenoyl)-glycero-3-phospho-(1′-sn-glycerol); 1-hexadecyl-2-eicosanoyl-glycero-3-phospho-(1′-sn-glycerol); 1-hexadecyl-2-(9Z-octadecenoyl)-glycero-3-phospho-(1′-sn-glycerol); 1-hexadecyl-glycero-3-phospho-(1′-sn-glycerol); 1,2-didocosanoyl-sn-glycero-3-phospho-(1′-sn-glycerol); 1-docosanoyl-2-(11Z-eicosenoyl)-glycero-3-phospho-(1′-sn-glycerol); 1-docosanoyl-2-(9Z-octadecenoyl)-glycero-3-phospho-(1′-sn-glycerol); 1-eicosanoyl-2-docosanoyl-glycero-3-phospho-(1′-sn-glycerol); 1-eicosanoyl-2-(11Z,14Z-eicosadienoyl)-glycero-3-phospho-(1′-sn-glycerol); 1-eicosanoyl-2-(11Z-eicosenoyl)-glycero-3-phospho-(1′-sn-glycerol); 1-eicosanoyl-2-(6Z,9Z,12Z,15Z-octadecatetraenoyl)-glycero-3-phospho-(1′-sn-glycerol); 1-eicosanoyl-2-(9Z,12Z-octadecadienoyl)-glycero-3-phospho-(1′-sn-glycerol); 1-eicosanoyl-2-(9Z-octadecenoyl)-glycero-3-phospho-(1′-sn-glycerol); 1-eicosanoyl-2-octadecanoyl-glycero-3-phospho-(1′-sn-glycerol); 1-nonadecanoyl-2-hexadecanoyl-glycero-3-phospho-(1′-sn-glycerol); 1-(9Z,12Z,15Z-octadecatrienoyl)-2-hexadecanoyl-glycero-3-phospho-(1′-sn-glycerol); 1,2-di-(9Z,12Z-octadecadienoyl)-sn-glycero-3-phospho-(1′-sn-glycerol); 1-(9Z-octadecenoyl)-2-docosanoyl-glycero-3-phospho-(1′-sn-glycerol); 1-(9Z-octadecenoyl)-2-(11Z-eicosenoyl)-glycero-3-phospho-(1′-sn-glycerol); 1-(9Z-octadecenoyl)-2-eicosanoyl-glycero-3-phospho-(1′-sn-glycerol); 1-(9Z-octadecenoyl)-2-(9Z,12Z-octadecadienoyl)-glycero-3-phospho-(1′-sn-glycerol); 1-(9Z-octadecenoyl)-2-octadecanoyl-glycero-3-phospho-(1′-sn-glycerol); 1-(9Z-octadecenoyl)-2-hexadecanoyl-glycero-3-phospho-(1′-sn-glycerol); 1-octadecanoyl-2-(11Z,14Z-eicosadienoyl)-glycero-3-phospho-(1′-sn-glycerol); 1-octadecanoyl-2-(11Z-eicosenoyl)-glycero-3-phospho-(1′-sn-glycerol); 1-octadecanoyl-2-eicosanoyl-glycero-3-phospho-(1′-sn-glycerol); 1-heptadecanoyl-2-eicosanoyl-glycero-3-phospho-(1′-sn-glycerol); 1,2-di-(9Z-hexadecenoyl)-sn-glycero-3-phospho-(1′-sn-glycerol); 1-hexadecanoyl-2-(7Z,10Z,13Z,16Z-docosatetraenoyl)-glycero-3-phospho-(1′-sn-glycerol); 1-hexadecanoyl-2-docosanoyl-glycero-3-phospho-(1′-sn-glycerol); 1-hexadecanoyl-2-(11Z-eicosenoyl)-glycero-3-phospho-(1′-sn-glycerol); 1-hexadecanoyl-2-eicosanoyl-glycero-3-phospho-(1′-sn-glycerol); 1-hexadecanoyl-2-nonadecanoyl-glycero-3-phospho-(1′-sn-glycerol); 1-hexadecanoyl-2-(9Z,12Z,15Z-octadecatrienoyl)-glycero-3-phospho-(1′-sn-glycerol); 1-hexadecanoyl-2-tetradecanoyl-glycero-3-phospho-(1′-sn-glycerol); 1,2-ditridecanoyl-sn-glycero-3-phospho-(1′-sn-glycerol); 1-dodecanoyl-2-tetradecanoyl-glycero-3-phospho-(1′-sn-glycerol); 1,2-di-(4Z,7Z,10Z,13Z,16Z,19Z-docosahexaenoyl)-sn-glycero-3-phospho-(1′-sn-glycerol); 1-(7Z,10Z,13Z,16Z-docosatetraenoyl)-2-(4Z,7Z,10Z,13Z,16Z,19Z-docosahexaenoyl)-glycero-3-phospho-(1′-sn-glycerol); 1-docosanoyl-2-(4Z,7Z,10Z,13Z,16Z,19Z-docosahexaenoyl)-glycero-3-phospho-(1′-sn-glycerol); 1,2-di-(9Z,12Z,15Z-octadecatrienoyl)-sn-glycero-3-phospho-(1′-sn-glycerol); 1-octadecanoyl-2-docosanoyl-glycero-3-phospho-(1′-sn-glycerol); 1-octadecanoyl-2-(9Z,12Z-octadecadienoyl)-glycero-3-phospho-(1′-sn-glycerol); 1-(9Z-heptadecenoyl)-glycero-3-phospho-(1′-sn-glycerol); 1-hexadecanoyl-2-(9Z,12Z-octadecadienoyl)-glycero-3-phospho-(1′-sn-glycerol); 1-tetradecanoyl-2-(9Z-octadecenoyl)-glycero-3-phospho-(1′-sn-glycerol); 1-(1Z-eicosenyl)-2-(4Z,7Z,10Z,13Z,16Z,19Z-docosahexaenoyl)-glycero-3-phospho-(1′-sn-glycerol); 1-eicosyl-2-(4Z,7Z,10Z,13Z,16Z,19Z-docosahexaenoyl)-glycero-3-phospho-(1′-sn-glycerol); 1,2-di-(9Z-octadecenoyl)-sn-glycero-3-phospho-(1′-sn-glycerol); 1,2-dihexadecanoyl-sn-glycero-3-phospho-(1′-sn-glycerol); 2,3-diphytanyl-sn-glycero-1-phospho-(1′-sn-glycerol); 2-hexadecanoyl-3-lyso-sn-glycero-1-phospho-(2′-(9Z-octadecenoyl)-3′-lyso-1′-sn-glycerol); Bis-[2-(9Z-octadecenoyl)-3-lyso-sn-glycero]-1-phosphate; 2-(9Z-hexadecenoyl)-3-lyso-sn-glycero-1-phospho-(2′-(9Z-octadecenoyl)-3′-lyso-1′-sn-glycerol); 1-hexadecanoyl-2-(11R, 12S-methylene-octadecanoyl)-sn-glycero-3-phospho-(1′-sn-glycerol); 1-hexadecanoyl-2-(9R, 11 S-methylene-hexadecanoyl)-sn-glycero-3-phospho-(1′-sn-glycerol); 1-tetradecanoyl-2-(9R, 11 S-methylene-hexadecanoyl)-sn-glycero-3-phospho-(1′-sn-glycerol); 1-pentadecanoyl-2-(9R, 11 S-methylene-hexadecanoyl)-sn-glycero-3-phospho-(1′-sn-glycerol); 1-decanoyl-2-tetradecanoyl-glycero-3-phospho-(1′-rac-glycerol); 1-(Z)-alk-1-enyl-2-acyl-sn-glycero-3-phospho-(1′-sn-glycerol); 1-acyl-sn-glycero-3-phospho-(1′-sn-glycerol); 1-(1Z-alkenyl)-sn-glycero-3-phospho-(1′-sn-glycerol); 1-alkyl-2-acyl-sn-glycero-3-phospho-(1′-sn-glycerol); 1-alkyl-sn-glycero-3-phospho-(1′-sn-glycerol); 1,2-(13-methyl-tetradecanoyl)-sn-glycero-3-phospho-(1′-sn-glycerol); 1-(13-methyl-tetradecanoyl)-2-(12-methyl-tetradecanoyl)-sn-glycero-3-phospho-(1′-sn-glycerol); 1-(12-methyl-tetradecanoyl)-2-(13-methyl-tetradecanoyl)-sn-glycero-3-phospho-(1′-sn-glycerol); and/or 1,2-(12-methyl-tetradecanoyl)-sn-glycero-3-phospho-(1′-sn-glycerol).

### Sphingolipid

In some embodiments, the lipid of the disclosure may be a sphingolipid. As used herein, the term “sphingolipid” refers to a lipid composed of one molecule of the long-chain amino alcohol sphingosine (also called 4-sphingenine) or a derivative thereof, one molecule of a long chain fatty acid, and a polar head group that may be joined by a glycosidic or a phosphodiester bond.

In some embodiments, the sphingolipid may be a ceramide, a cerebroside, a sphingomyelin, or a ganglioside. In some embodiments, the sphingolipid may be a ceramide comprising a fatty acid attached in amide linkage to the —NH₂ group on C-2.

In some embodiments, the lipid of the disclosure may be sphingomyelins which contain phosphocholine or phosphoethanolamine as their polar head group. Sphingomyelins are present in the plasma membranes of animal cells and are especially prominent in myelin, a membranous sheath that surrounds and insulates the axons of some neurons.

In some embodiments, the lipid of the disclosure may be glycosphingolipids. Glycosphingolipids have headgroups with one or more sugars connected directly to the OH at C-1 of the ceramide moiety. In some embodiments, lipids of the disclosure may be cerebrosides.

In some embodiments, the lipids of the disclosure may be cerebrosides. Cerebrosides may have a single sugar linked to ceramide. When two or more sugars are linked to the ceramide, the lipid may be referred to as a globoside.

In some embodiments, the lipids of the disclosure may be a ganglioside. Gangliosides, the most complex sphingolipids, have oligosaccharides as their polar head groups and one or more residues of N-acetylneuraminic acid (Neu5Ac), a sialic acid at the termini.

In some embodiments, the lipid of the disclosure may be derivative of sphingolipid. Non-limiting examples of a sphingolipid derivatives are N-oleoyl-D-erythro-sphingosylphosphorylcholine; 2-[N-(13-methyl-tetradecanoyl)-3R-hydroxy-15-methylhexadecane]-1-sulfonic acid; (2R,3R)-2-amino-3-hydroxy-15-methylhexadecane-1-sulfonic acid; (2R,3R)-3-Hydroxy-15-methyl-2-[(13-methyltetradecanoyl)amino]-1-hexadecanesulfonic acid; (2R,3R)-3-Hydroxy-15-methyl-2-[(2-oxo-13-methyltetradecanoyl)amino]-1-hexadecanesulfonic acid; (2R,3R,26R,27S)-2,27-diamino-1,3,26-trihydroxyoctacosan-11-one; (2R,3R,26R,27S)-2,27-diamino-3,26,28-trihydroxyoctacosan-11-one; (2R,3R,4E)-3-Hydroxy-2-([(3R)-3-hydroxy-15-methylhexadecanoyl]amino))-15-methyl-4-hexadecene-1-sulfonic acid; (2R,3R,4E)-3-Hydroxy-2-([(3R)-3-hydroxy-15-methylhexadecanoyl]amino))-15-methyl-hexadecane-1-sulfonic acid; (2R,3R,4E)-3-Hydroxy-2-(palmitoylamino)-4-octadecene-1-sulfonic acid; (2S,27S-Diamino-3R,26S-dihydroxy-18-oxooctacosyl)beta-D-galactopyranoside; (2S,27S-Diamino-3R,26S-dihydroxy-18-oxooctacosyl)beta-D-glucopyranoside; (2S,3R)-2-amino-2-hydroxymethyl-3-hydroxysulfonyloxy-12-oxo-octadecanoic acid; (2S,3R,4E)-2-amino-3-hydroxyoctadec-4-en-1-yl beta-D-galactopyranoside 6-(hydrogen sulfate); (2S,3R,4E,8E)-3-Hydroxy-2-[methyl(stearoyl)amino]-4,8-octadecadien-1-yl hydrogen sulfate; (2S,3R,5Z,8Z,11Z,14Z,17Z,20Z,28R,29S)-2,29-diaminotriaconta-5,8,11,14,17,20-hexaene-3,28-diol; (2S,3S,26R,27S)-2,27-diamino-3,26,28-trihydroxyoctacosan-11-one; (2S,3S,4R)-2-amino-3,4-dihydroxyoctadecyl dihydrogen phosphate; (2S,4E)-3-Hydroxy-2-[(2-hydroxy-13-methyltetradecanoyl)amino]-15-methyl-4-hexadecene-1-sulfonic acid; (3′-sulfo)Galbeta-N-(acyl)-sphing-4-enine; (9aS)-6S-(deca-1,3E-dienyl)-4S-methyloctahydro-1H-quinolizin-3R-ol; 11-(5S-hydroxy-6S-methylpiperidin-2R-yl)undecan-3-one; 14-methyl-hexadecasphing-4E-enine; 15-methyl-hexadecasphing-4E-enine; 1-beta-galactosyl-sphing-4-enine; 1-beta-glucosyl-sphing-4-enine; 1-deoxy-11E-tetradecasphingenine; 1-deoxy-11Z-hexadecasphingenine; 1-deoxy-15-hexadecasphingenine; 1-deoxy-9Z-hexadecasphingenine; 1-deoxy-hexadecasphinga-11E,15-dienine; 1-deoxy-hexadecasphinga-7Z,15-dienine; 1-deoxy-sphinga-5E,9Z,12Z,15Z-tetraenine; 1-deoxy-sphinga-6Z,9Z,12Z,15Z-tetraenine; 1-deoxy-sphinganine; 1-deoxy-tetradecasphing-13-enine; 1-deoxy-tetradecasphinga-11E,13E-dienine; 1-deoxy-tetradecasphinganine; 1-O-(6′-(N-methylphosphoethanolaminyl)-beta-D-glucopyranosyl)-(N-(2R-hydroxy-docosanoyl)-4R-hydroxy-16-methyl-hexadecasphinganine); 1-O-(6′-(N-methylphosphoethanolaminyl)-beta-D-glucopyranosyl)-(N-(2R-hydroxy-tetracosanoyl)-4R-hydroxy-16-methyl-hexadecasphinganine); 1-O-(6′-(N-methylphosphoethanolaminyl)-beta-D-glucopyranosyl)-(N-(2R-hydroxy-tricosanoyl)-4R-hydroxy-16-methyl-hexadecasphinganine); 1-O-(6′-phosphoethanolaminy-beta-D-glucopyranosyl)-(N-(2R-hydroxy-docosanoyl)-4R-hydroxy-16-methyl-hexadecasphinganine); 1-O-(6′-phosphoethanolaminy-beta-D-glucopyranosyl)-(N-(2R-hydroxy-tetracosanoyl)-4R-hydroxy-16-methyl-hexadecasphinganine); 1-O-docosanoyl-N-(hexadecanoyl)-sphing-4-enine; 1-O-docosanoyl-N-(octadecanoyl)-sphing-4-enine; 1-O-eicosanoyl-N-(hexadecanoyl)-sphing-4-enine; 1-O-eicosanoyl-N-(octadecanoyl)-sphing-4-enine; 1-O-hexacosanoyl-N-(hexadecanoyl)-sphing-4-enine; 1-O-hexacosanoyl-N-(octadecanoyl)-sphing-4-enine; 1-O-hexadecanoyl-N-(hexadecanoyl)-sphing-4-enine; 1-O-hexadecanoyl-N-(octadecanoyl)-sphing-4-enine; 1-O-melibiosoyl-(N-(2R-hydroxy-docosanoyl)-4R-hydroxy-16-methyl-heptasphinganine); 1-O-melibiosoyl-(N-(2R-hydroxy-docosanoyl)-4R-hydroxy-16-methyl-hexadecasphinganine); 1-O-melibiosoyl-(N-(2R-hydroxy-docosanoyl)-4R-hydroxy-16-methyl-sphing-6E-enine); 1-O-melibiosoyl-(N-(2R-hydroxy-docosanoyl)-4R-hydroxy-16-methyl-sphinganine); 1-O-melibiosoyl-(N-(2R-hydroxy-docosanoyl)-4R-hydroxy-17-methyl-sphing-6E-enine); 1-O-melibiosoyl-(N-(2R-hydroxy-docosanoyl)-4R-hydroxy-17-methyl-sphinganine); 1-O-melibiosoyl-(N-(2R-hydroxy-docosanoyl)-4R-hydroxy-heptadecasphinganine); 1-O-melibiosoyl-(N-(2R-hydroxy-docosanoyl)-4R-hydroxy-sphinganine); 1-O-melibiosoyl-(N-(2R-hydroxy-heneicosanoyl)-4R-hydroxy-16-methyl-heptasphinganine); 1-O-melibiosoyl-(N-(2R-hydroxy-heneicosanoyl)-4R-hydroxy-16-methyl-sphing-6E-enine); 1-O-melibiosoyl-(N-(2R-hydroxy-heneicosanoyl)-4R-hydroxy-17-methyl-sphing-6E-enine); 1-O-melibiosoyl-(N-(2R-hydroxy-heneicosanoyl)-4R-hydroxy-sphinganine); 1-O-melibiosoyl-(N-(2R-hydroxy-tricosanoyl)-4R-hydroxy-16-methyl-heptasphinganine); 1-O-melibiosoyl-(N-(2R-hydroxy-tricosanoyl)-4R-hydroxy-sphinganine); 1-O-octacosanoyl-N-(octadecanoyl)-sphing-4-enine; 1-O-octadecanoyl-N-(32-(9Z,12Z-octadecadienoyloxy)-dotriacontanoyl)-4-hydroxysphinganine; 1-O-octadecanoyl-N-(hexadecanoyl)-sphing-4-enine; 1-O-octadecanoyl-N-(octadecanoyl)-sphing-4-enine; 1-O-pentacosanoyl-N-(hexadecanoyl)-sphing-4-enine; 1-O-tetracosanoyl-N-(30-(9Z,12Z-octadecadienoyloxy)-triacontanoyl)-6-hydroxy-sphing-4E-enine; 1-O-tetracosanoyl-N-(hexadecanoyl)-sphing-4-enine; 1-O-tetracosanoyl-N-(octadecanoyl)-sphing-4-enine; 1-O-tetradecanoyl-N-(hexadecanoyl)-sphing-4-enine; 1-O-tetradecanoyl-N-(octadecanoyl)-sphing-4-enine; 1-O-tricosanoyl-N-(hexadecanoyl)-sphing-4-enine; 1-O-tricosanoyl-N-(octadecanoyl)-sphing-4-enine; 2-[N-(2-hydroxy-13-methyl-tetradecanoyl)-3R,4-dihydroxy-15-methylhexadecane]-1-sulfonic acid; 2-amino-14,16-dimethyloctadecan-3-ol; 2-carboxy-2-amino-3-O-(13′-methyltetradecanoyl)-4-hydroxy-17-methyloctadec-5-ene-1-sulfonic acid; 2-glycosyl-23-amino-3,22-dihydroxytetracosane; 2R-amino-3E-octadecene-1,5-diol; 2S-((11 S-hydroxy-12-methyltetradecyl)-4S-(hydroxymethyl)azetidin-3R-ol; 2S-((11 S-hydroxy-13-methyltetradecyl)-4S-(hydroxymethyl)azetidin-3R-ol; 2S-(hydroxymethyl)-4S-(12-methyloctadecyl)azetidin-3R-ol; 2S-acetamido-3R,4R,5S,14R-tetrahydroxyeicos-6E-enoic acid; 2S-amino-1,3S-dihydroxyheptadec-6-ene-4R-sulfate; 2S-amino-1,3S-dihydroxyoctadec-6-ene-4R-sulfate; 2S-amino-3R,4R,5S,14R-tetrahydroxyeicos-6E-enoic acid; 2S-amino-3R,4R,5S-trihydroxy-2-(hydroxymethyl)-14-oxo-eicos-6E-enoic acid; 2S-amino-3R,4R,5S-trihydroxy-2-methyl-14-oxo-eicos-6E-enoic acid; 2S-amino-5,9,13,17-tetramethyl-8E,16-octadecadiene-1,3R,14-triol; 2S-amino-5S-acetoxy-3R,4R,14R-trihydroxyeicos-6E-enoic acid; 2S-amino-octadeca-4E,6E-diene-1,3R-diol; 2S-aminooctadecane-1,3R-diol; 2S-aminooctadecane-3S,5S-diol; 2S-dimethylaminooctadecane-1,3R-diol; 2S-guanidino-3R,4R,5S,14R-tetrahydroxyicos-6E-enoic acid; 3-dehydrosphinganine; 3-keto-sphing-4-enine; 4R-hydroxy-heptadeasphinganine; 4R-hydroxysphing-8E-enine; 4R-hydroxy-sphing-8Z-enine; 4R-hydroxysphinganine; 5S-(5S-hydroxyoctyl)-2S-methyldecahydroquinolin-3R-ol; 6-hydroxysphing-4E-enine; 6R-(8-hydroxydecyl)-2R-(hydroxymethyl)-piperidin-3R-ol; 6S-((deca-1E,3E,5E-trienyl)-2R-(hydroxymethyl)-piperidin-3R-ol; 9-methyl-sphinga-4E,8E,10E-trienine; 9-methyl-sphinga-4E,8E-dienine; 9-OAc-KDNalpha2-3Galbeta1-3GalNAcbeta1-4(KDNalpha2-3)Galbeta1-4Glcbeta-Cer; 9-OAc-NeuAcalpha2-3Galbeta1-3GalNAcbeta1-4(NeuAcalpha2-3)Galbeta1-4Glcbeta-Cer; 9-OAc-NeuAcalpha2-3Galbeta1-3GalNAcbeta1-4(NeuAcalpha2-3)Galbeta1-4Glcbeta-Cer(d18:1/16:0); 9-OAc-NeuAcalpha2-3Galbeta1-3GalNAcbeta1-4(NeuAcalpha2-3)Galbeta1-4Glcbeta-Cer(d18:1/18:0); 9-OAc-NeuAcalpha2-3Galbeta1-3GalNAcbeta1-4(NeuAcalpha2-3)Galbeta1-4Glcbeta-Cer(d18:1/20:0); 9-OAc-NeuAcalpha2-3Galbeta1-3GalNAcbeta1-4(NeuAcalpha2-3)Galbeta1-4Glcbeta-Cer(d18:1/22:0); 9-OAc-NeuAcalpha2-3Galbeta1-3GalNAcbeta1-4(NeuAcalpha2-3)Galbeta1-4Glcbeta-Cer(d18:1/24:0); 9-OAc-NeuAcalpha2-3Galbeta1-3GalNAcbeta1-4(NeuAcalpha2-3)Galbeta1-4Glcbeta-Cer(d18:1/24:1(15Z)); 9-OAc-NeuAcalpha2-3Galbeta1-3GalNAcbeta1-4(NeuAcalpha2-3)Galbeta1-4Glcbeta-Cer(d18:1/26:0); 9-OAc-NeuAcalpha2-3Galbeta1-3GalNAcbeta1-4(NeuAcalpha2-3)Galbeta1-4Glcbeta-Cer(d18:1/26:1(17Z)); 9-OAc-NeuAcalpha2-8NeuAcalpha2-8NeuAcalpha2-3Galbeta1-4Glcbeta-Cer; 9-OAc-NeuAcalpha2-8NeuAcalpha2-8NeuAcalpha2-3Galbeta1-4Glcbeta-Cer(d18:1/16:0); 9-OAc-NeuAcalpha2-8NeuAcalpha2-8NeuAcalpha2-3Galbeta1-4Glcbeta-Cer(d18:1/18:0); 9-OAc-NeuAcalpha2-8NeuAcalpha2-8NeuAcalpha2-3Galbeta1-4Glcbeta-Cer(d18:1/20:0); 9-OAc-NeuAcalpha2-8NeuAcalpha2-8NeuAcalpha2-3Galbeta1-4Glcbeta-Cer(d18:1/22:0); 9-OAc-NeuAcalpha2-8NeuAcalpha2-8NeuAcalpha2-3Galbeta1-4Glcbeta-Cer(d18:1/24:0); 9-OAc-NeuAcalpha2-8NeuAcalpha2-8NeuAcalpha2-3Galbeta1-4Glcbeta-Cer(d18:1/24:1(15Z)); 9-OAc-NeuAcalpha2-8NeuAcalpha2-8NeuAcalpha2-3Galbeta1-4Glcbeta-Cer(d18:1/26:0); 9-OAc-NeuAcalpha2-8NeuAcalpha2-8NeuAcalpha2-3Galbeta1-4Glcbeta-Cer(d18:1/26:1(17Z)); EtnP-6GlcNAcbeta1-3Manbeta1-4Glcbeta-Cer; Fucalpha1-2(Galalpha1-3)Galbeta1-4GlcNAcbeta1-3(Fucalpha1-2Galbeta1-4GlcNAcbeta1-6)Galbeta1-4GlcNAcbeta1-3Galbeta1-4Glcbeta-Cer; Fucalpha1-2(Galalpha1-3)Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/16:0); Fucalpha1-2(Galalpha1-3)Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/18:0); Fucalpha1-2(Galalpha1-3)Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/20:0); Fucalpha1-2(Galalpha1-3)Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/22:0); Fucalpha1-2(Galalpha1-3)Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/24:0); Fucalpha1-2(Galalpha1-3)Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/24:1(15Z)); Fucalpha1-2(Galalpha1-3)Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/26:0); Fucalpha1-2(Galalpha1-3)Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/26:1(17Z)); Fucalpha1-2(Galalpha1-3)Galbeta1-4GlcNAcbeta1-3(Galalpha1-3Galbeta1-4GlcNAcbeta1-6)Galbeta1-4GlcNAcbeta1-3Galbeta1-4Glcbeta-Cer; Fucalpha1-2(Galalpha1-3)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/16:0); Fucalpha1-2(Galalpha1-3)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/18:0); Fucalpha1-2(Galalpha1-3)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/20:0); Fucalpha1-2(Galalpha1-3)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/22:0); Fucalpha1-2(Galalpha1-3)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:0); Fucalpha1-2(Galalpha1-3)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:1(15Z)); Fucalpha1-2(Galalpha1-3)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:0); Fucalpha1-2(Galalpha1-3)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:1(17Z)); Fucalpha1-2(Galalpha1-3)Galbeta1-4GlcNAcbeta1-6(Galalpha1-3(Fucalpha1-2)Galbeta1-4GlcNAcbeta1-3Galbeta1-4GlcNAcbeta1-3)Galbeta1-4GlcNAcbeta1-3Galbeta1-4Glcbeta-Cer; Fucalpha1-2(Galalpha1-3)Galbeta1-4GlcNAcbeta1-6(Galalpha1-3(Fucalpha1-2)Galbeta1-4GlcNAcbeta1-3Galbeta1-4GlcNAcbeta1-3)Galbeta1-4GlcNAcbeta1-3Galbeta1-4Glcbeta-Cer(d18:1/16:0); Fucalpha1-2(Galalpha1-3)Galbeta1-4GlcNAcbeta1-6(Galalpha1-3(Fucalpha1-2)Galbeta1-4GlcNAcbeta1-3Galbeta1-4GlcNAcbeta1-3)Galbeta1-4GlcNAcbeta1-3Galbeta1-4Glcbeta-Cer(d18:1/18:0); Fucalpha1-2(Galalpha1-3)Galbeta1-4GlcNAcbeta1-6(Galalpha1-3(Fucalpha1-2)Galbeta1-4GlcNAcbeta1-3Galbeta1-4GlcNAcbeta1-3)Galbeta1-4GlcNAcbeta1-3Galbeta1-4Glcbeta-Cer(d18:1/20:0); Fucalpha1-2(Galalpha1-3)Galbeta1-4GlcNAcbeta1-6(Galalpha1-3(Fucalpha1-2)Galbeta1-4GlcNAcbeta1-3Galbeta1-4GlcNAcbeta1-3)Galbeta1-4GlcNAcbeta1-3Galbeta1-4Glcbeta-Cer(d18:1/22:0); Fucalpha1-2(Galalpha1-3)Galbeta1-4GlcNAcbeta1-6(Galalpha1-3(Fucalpha1-2)Galbeta1-4GlcNAcbeta1-3Galbeta1-4GlcNAcbeta1-3)Galbeta1-4GlcNAcbeta1-3Galbeta1-4Glcbeta-Cer(d18:1/24:0); Fucalpha1-2(Galalpha1-3)Galbeta1-4GlcNAcbeta1-6(Galalpha1-3(Fucalpha1-2)Galbeta1-4GlcNAcbeta1-3Galbeta1-4GlcNAcbeta1-3)Galbeta1-4GlcNAcbeta1-3Galbeta1-4Glcbeta-Cer(d18:1/24:1(15Z)); Fucalpha1-2(Galalpha1-3)Galbeta1-4GlcNAcbeta1-6(Galalpha1-3(Fucalpha1-2)Galbeta1-4GlcNAcbeta1-3Galbeta1-4GlcNAcbeta1-3)Galbeta1-4GlcNAcbeta1-3Galbeta1-4Glcbeta-Cer(d18:1/26:0); Fucalpha1-2(Galalpha1-3)Galbeta1-4GlcNAcbeta1-6(Galalpha1-3(Fucalpha1-2)Galbeta1-4GlcNAcbeta1-3Galbeta1-4GlcNAcbeta1-3)Galbeta1-4GlcNAcbeta1-3Galbeta1-4Glcbeta-Cer(d18:1/26:1(17Z)); Fucalpha1-2(GalNAcalpha1-3)Galbeta1-4GlcNAcbeta1-3(Fucalpha1-2Galbeta1-4GlcNAcbeta1-6)Galbeta1-4GlcNAcbeta1-3Galbeta1-4Glcbeta-Cer; Fucalpha1-2(GalNAcalpha1-3)Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/16:0); Fucalpha1-2(GalNAcalpha1-3)Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/18:0); Fucalpha1-2(GalNAcalpha1-3)Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/20:0); Fucalpha1-2(GalNAcalpha1-3)Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/22:0); Fucalpha1-2(GalNAcalpha1-3)Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/24:0); Fucalpha1-2(GalNAcalpha1-3)Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/24:1(15Z)); Fucalpha1-2(GalNAcalpha1-3)Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/26:0); Fucalpha1-2(GalNAcalpha1-3)Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/26:1(17Z)); Fucalpha1-2(GalNAcalpha1-3)Galbeta1-4GlcNAcbeta1-3(Galbeta1-4GlcNAcbeta1-6)Galbeta1-4GlcNAcbeta1-3Galbeta1-4Glcbeta-Cer; Fucalpha1-2(GalNAcalpha1-3)Galbeta1-4GlcNAcbeta1-3(Galbeta1-4GlcNAcbeta1-6)Galbeta1-4GlcNAcbeta1-3Galbeta1-4Glcbeta-Cer(d18:1/16:0); Fucalpha1-2(GalNAcalpha1-3)Galbeta1-4GlcNAcbeta1-3(Galbeta1-4GlcNAcbeta1-6)Galbeta1-4GlcNAcbeta1-3Galbeta1-4Glcbeta-Cer(d18:1/18:0); Fucalpha1-2(GalNAcalpha1-3)Galbeta1-4GlcNAcbeta1-3(Galbeta1-4GlcNAcbeta1-6)Galbeta1-4GlcNAcbeta1-3Galbeta1-4Glcbeta-Cer(d18:1/20:0); Fucalpha1-2(GalNAcalpha1-3)Galbeta1-4GlcNAcbeta1-3(Galbeta1-4GlcNAcbeta1-6)Galbeta1-4GlcNAcbeta1-3Galbeta1-4Glcbeta-Cer(d18:1/22:0); Fucalpha1-2(GalNAcalpha1-3)Galbeta1-4GlcNAcbeta1-3(Galbeta1-4GlcNAcbeta1-6)Galbeta1-4GlcNAcbeta1-3Galbeta1-4Glcbeta-Cer(d18:1/24:0); Fucalpha1-2(GalNAcalpha1-3)Galbeta1-4GlcNAcbeta1-3(Galbeta1-4GlcNAcbeta1-6)Galbeta1-4GlcNAcbeta1-3Galbeta1-4Glcbeta-Cer(d18:1/24:1(15Z)); Fucalpha1-2(GalNAcalpha1-3)Galbeta1-4GlcNAcbeta1-3(Galbeta1-4GlcNAcbeta1-6)Galbeta1-4GlcNAcbeta1-3Galbeta1-4Glcbeta-Cer(d18:1/26:0); Fucalpha1-2(GalNAcalpha1-3)Galbeta1-4GlcNAcbeta1-3(Galbeta1-4GlcNAcbeta1-6)Galbeta1-4GlcNAcbeta1-3Galbeta1-4Glcbeta-Cer(d18:1/26:1(17Z)); Fucalpha1-2(GalNAcalpha1-3)Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer; Fucalpha1-2(GalNAcalpha1-3)Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/16:0); Fucalpha1-2(GalNAcalpha1-3)Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/18:0); Fucalpha1-2(GalNAcalpha1-3)Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/20:0); Fucalpha1-2(GalNAcalpha1-3)Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/22:0); Fucalpha1-2(GalNAcalpha1-3)Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/24:0); Fucalpha1-2(GalNAcalpha1-3)Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/24:1(15Z)); Fucalpha1-2(GalNAcalpha1-3)Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/26:0); Fucalpha1-2(GalNAcalpha1-3)Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/26:1(17Z)); Fucalpha1-2(GalNAcalpha1-3)Galbeta1-4GlcNAcbeta1-6(Galbeta1-4GlcNAcbeta1-3)Galbeta1-4GlcNAcbeta1-3Galbeta1-4Glcbeta-Cer; Fucalpha1-2(GalNAcalpha1-3)Galbeta1-4GlcNAcbeta1-6(Galbeta1-4GlcNAcbeta1-3)Galbeta1-4GlcNAcbeta1-3Galbeta1-4Glcbeta-Cer(d18:1/16:0); Fucalpha1-2(GalNAcalpha1-3)Galbeta1-4GlcNAcbeta1-6(Galbeta1-4GlcNAcbeta1-3)Galbeta1-4GlcNAcbeta1-3Galbeta1-4Glcbeta-Cer(d18:1/18:0); Fucalpha1-2(GalNAcalpha1-3)Galbeta1-4GlcNAcbeta1-6(Galbeta1-4GlcNAcbeta1-3)Galbeta1-4GlcNAcbeta1-3Galbeta1-4Glcbeta-Cer(d18:1/20:0); Fucalpha1-2(GalNAcalpha1-3)Galbeta1-4GlcNAcbeta1-6(Galbeta1-4GlcNAcbeta1-3)Galbeta1-4GlcNAcbeta1-3Galbeta1-4Glcbeta-Cer(d18:1/22:0); Fucalpha1-2(GalNAcalpha1-3)Galbeta1-4GlcNAcbeta1-6(Galbeta1-4GlcNAcbeta1-3)Galbeta1-4GlcNAcbeta1-3Galbeta1-4Glcbeta-Cer(d18:1/24:0); Fucalpha1-2(GalNAcalpha1-3)Galbeta1-4GlcNAcbeta1-6(Galbeta1-4GlcNAcbeta1-3)Galbeta1-4GlcNAcbeta1-3Galbeta1-4Glcbeta-Cer(d18:1/24:1(15Z)); Fucalpha1-2(GalNAcalpha1-3)Galbeta1-4GlcNAcbeta1-6(Galbeta1-4GlcNAcbeta1-3)Galbeta1-4GlcNAcbeta1-3Galbeta1-4Glcbeta-Cer(d18:1/26:0); Fucalpha1-2(GalNAcalpha1-3)Galbeta1-4GlcNAcbeta1-6(Galbeta1-4GlcNAcbeta1-3)Galbeta1-4GlcNAcbeta1-3Galbeta1-4Glcbeta-Cer(d18:1/26:1(17Z)); Fucalpha1-2Galalpha1-3Galalpha1-4Galbeta1-4Glcbeta-Cer; Fucalpha1-2Galalpha1-3Galalpha1-4Galbeta1-4Glcbeta-Cer(d18:1/16:0); Fucalpha1-2Galalpha1-3Galalpha1-4Galbeta1-4Glcbeta-Cer(d18:1/18:0); Fucalpha1-2Galalpha1-3Galalpha1-4Galbeta1-4Glcbeta-Cer(d18:1/20:0); Fucalpha1-2Galalpha1-3Galalpha1-4Galbeta1-4Glcbeta-Cer(d18:1/22:0); Fucalpha1-2Galalpha1-3Galalpha1-4Galbeta1-4Glcbeta-Cer(d18:1/24:0); Fucalpha1-2Galalpha1-3Galalpha1-4Galbeta1-4Glcbeta-Cer(d18:1/24:1(15Z)); Fucalpha1-2Galalpha1-3Galalpha1-4Galbeta1-4Glcbeta-Cer(d18:1/26:0); Fucalpha1-2Galalpha1-3Galalpha1-4Galbeta1-4Glcbeta-Cer(d18:1/26:1(17Z)); Fucalpha1-2Galalpha1-3Galbeta1-4Glcbeta-Cer; Fucalpha1-2Galalpha1-3Galbeta1-4Glcbeta-Cer(d18:1/16:0); Fucalpha1-2Galalpha1-3Galbeta1-4Glcbeta-Cer(d18:1/18:0); Fucalpha1-2Galalpha1-3Galbeta1-4Glcbeta-Cer(d18:1/20:0); Fucalpha1-2Galalpha1-3Galbeta1-4Glcbeta-Cer(d18:1/22:0); Fucalpha1-2Galalpha1-3Galbeta1-4Glcbeta-Cer(d18:1/24:0); Fucalpha1-2Galalpha1-3Galbeta1-4Glcbeta-Cer(d18:1/24:1(15Z)); Fucalpha1-2Galalpha1-3Galbeta1-4Glcbeta-Cer(d18:1/26:0); Fucalpha1-2Galalpha1-3Galbeta1-4Glcbeta-Cer(d18:1/26:1(17Z)); Fucalpha1-2Galbeta1-3(Fucalpha1-4)GlcNAcbeta1-3Galbeta1-4Glcbeta-Cer; Fucalpha1-2Galbeta1-3(Fucalpha1-4)GlcNAcbeta1-3Galbeta1-4Glcbeta-Cer(d18:1/16:0); Fucalpha1-2Galbeta1-3(Fucalpha1-4)GlcNAcbeta1-3Galbeta1-4Glcbeta-Cer(d18:1/18:0); Fucalpha1-2Galbeta1-3(Fucalpha1-4)GlcNAcbeta1-3Galbeta1-4Glcbeta-Cer(d18:1/20:0); Fucalpha1-2Galbeta1-3(Fucalpha1-4)GlcNAcbeta1-3Galbeta1-4Glcbeta-Cer(d18:1/22:0); Fucalpha1-2Galbeta1-3(Fucalpha1-4)GlcNAcbeta1-3Galbeta1-4Glcbeta-Cer(d18:1/24:0); Fucalpha1-2Galbeta1-3(Fucalpha1-4)GlcNAcbeta1-3Galbeta1-4Glcbeta-Cer(d18:1/24:1(15Z)); Fucalpha1-2Galbeta1-3(Fucalpha1-4)GlcNAcbeta1-3Galbeta1-4Glcbeta-Cer(d18:1/26:0); Fucalpha1-2Galbeta1-3(Fucalpha1-4)GlcNAcbeta1-3Galbeta1-4Glcbeta-Cer(d18:1/26:1(17Z)); Fucalpha1-2Galbeta1-3GalNAcalpha1-3(Fucalpha1-2)Galbeta1-4GlcNAcbeta1-3Galbeta1-4Glcbeta-Cer; Fucalpha1-2Galbeta1-3GalNAcalpha1-3(Fucalpha1-2)Galbeta1-4GlcNAcbeta1-3Galbeta1-4Glcbeta-Cer(d18:1/16:0); Fucalpha1-2Galbeta1-3GalNAcalpha1-3(Fucalpha1-2)Galbeta1-4GlcNAcbeta1-3Galbeta1-4Glcbeta-Cer(d18:1/18:0); Fucalpha1-2Galbeta1-3GalNAcalpha1-3(Fucalpha1-2)Galbeta1-4GlcNAcbeta1-3Galbeta1-4Glcbeta-Cer(d18:1/20:0); Fucalpha1-2Galbeta1-3GalNAcalpha1-3(Fucalpha1-2)Galbeta1-4GlcNAcbeta1-3Galbeta1-4Glcbeta-Cer(d18:1/22:0); Fucalpha1-2Galbeta1-3GalNAcalpha1-3(Fucalpha1-2)Galbeta1-4GlcNAcbeta1-3Galbeta1-4Glcbeta-Cer(d18:1/24:0); Fucalpha1-2Galbeta1-3GalNAcalpha1-3(Fucalpha1-2)Galbeta1-4GlcNAcbeta1-3Galbeta1-4Glcbeta-Cer(d18:1/24:1(15Z)); Fucalpha1-2Galbeta1-3GalNAcalpha1-3(Fucalpha1-2)Galbeta1-4GlcNAcbeta1-3Galbeta1-4Glcbeta-Cer(d18:1/26:0); Fucalpha1-2Galbeta1-3GalNAcalpha1-3(Fucalpha1-2)Galbeta1-4GlcNAcbeta1-3Galbeta1-4Glcbeta-Cer(d18:1/26:1(17Z)); Fucalpha1-2Galbeta1-3GalNAcalpha1-3(Fucalpha1-2)Galbeta1-4GlcNAcbeta1-3Galbeta1-4GlcNAcbeta1-3Galbeta1-4Glcbeta-Cer; Fucalpha1-2Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/16:0); Fucalpha1-2Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/18:0); Fucalpha1-2Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/20:0); Fucalpha1-2Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/22:0); Fucalpha1-2Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/24:0); Fucalpha1-2Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/24:1(15Z)); Fucalpha1-2Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/26:0); Fucalpha1-2Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/26:1(17Z)); Fucalpha1-2Galbeta1-3GalNAcbeta1-3Galalpha1-3Galbeta1-4Glcbeta-Cer; Fucalpha1-2Galbeta1-3GalNAcbeta1-3Galalpha1-3Galbeta1-4Glcbeta-Cer(d18:1/16:0); Fucalpha1-2Galbeta1-3GalNAcbeta1-3Galalpha1-3Galbeta1-4Glcbeta-Cer(d18:1/18:0); Fucalpha1-2Galbeta1-3GalNAcbeta1-3Galalpha1-3Galbeta1-4Glcbeta-Cer(d18:1/20:0); Fucalpha1-2Galbeta1-3GalNAcbeta1-3Galalpha1-3Galbeta1-4Glcbeta-Cer(d18:1/22:0); Fucalpha1-2Galbeta1-3GalNAcbeta1-3Galalpha1-3Galbeta1-4Glcbeta-Cer(d18:1/24:0); Fucalpha1-2Galbeta1-3GalNAcbeta1-3Galalpha1-3Galbeta1-4Glcbeta-Cer(d18:1/24:1(15Z)); Fucalpha1-2Galbeta1-3GalNAcbeta1-3Galalpha1-3Galbeta1-4Glcbeta-Cer(d18:1/26:0); Fucalpha1-2Galbeta1-3GalNAcbeta1-3Galalpha1-3Galbeta1-4Glcbeta-Cer(d18:1/26:1(17Z)); Fucalpha1-2Galbeta1-3GalNAcbeta1-3Galalpha1-4Galbeta1-4Glcbeta-Cer; Fucalpha1-2Galbeta1-3GalNAcbeta1-3Galalpha1-4Galbeta1-4Glcbeta-Cer(d18:1/16:0); Fucalpha1-2Galbeta1-3GalNAcbeta1-3Galalpha1-4Galbeta1-4Glcbeta-Cer(d18:1/18:0); Fucalpha1-2Galbeta1-3GalNAcbeta1-3Galalpha1-4Galbeta1-4Glcbeta-Cer(d18:1/20:0); Fucalpha1-2Galbeta1-3GalNAcbeta1-3Galalpha1-4Galbeta1-4Glcbeta-Cer(d18:1/22:0); Fucalpha1-2Galbeta1-3GalNAcbeta1-3Galalpha1-4Galbeta1-4Glcbeta-Cer(d18:1/24:0); Fucalpha1-2Galbeta1-3GalNAcbeta1-3Galalpha1-4Galbeta1-4Glcbeta-Cer(d18:1/24:1(15Z)); Fucalpha1-2Galbeta1-3GalNAcbeta1-3Galalpha1-4Galbeta1-4Glcbeta-Cer(d18:1/26:0); Fucalpha1-2Galbeta1-3GalNAcbeta1-3Galalpha1-4Galbeta1-4Glcbeta-Cer(d18:1/26:1(17Z)); Fucalpha1-2Galbeta1-3GalNAcbeta1-4(NeuAcalpha2-3)Galbeta1-4Glcbeta-Cer; Fucalpha1-2Galbeta1-3GalNAcbeta1-4(NeuAcalpha2-3)Galbeta1-4Glcbeta-Cer(d18:1/16:0); Fucalpha1-2Galbeta1-3GalNAcbeta1-4(NeuAcalpha2-3)Galbeta1-4Glcbeta-Cer(d18:1/18:0); Fucalpha1-2Galbeta1-3GalNAcbeta1-4(NeuAcalpha2-3)Galbeta1-4Glcbeta-Cer(d18:1/20:0); Fucalpha1-2Galbeta1-3GalNAcbeta1-4(NeuAcalpha2-3)Galbeta1-4Glcbeta-Cer(d18:1/22:0); Fucalpha1-2Galbeta1-3GalNAcbeta1-4(NeuAcalpha2-3)Galbeta1-4Glcbeta-Cer(d18:1/24:0); Fucalpha1-2Galbeta1-3GalNAcbeta1-4(NeuAcalpha2-3)Galbeta1-4Glcbeta-Cer(d18:1/24:1(15Z)); Fucalpha1-2Galbeta1-3GalNAcbeta1-4(NeuAcalpha2-3)Galbeta1-4Glcbeta-Cer(d18:1/26:0); Fucalpha1-2Galbeta1-3GalNAcbeta1-4(NeuAcalpha2-3)Galbeta1-4Glcbeta-Cer(d18:1/26:1(17Z)); Fucalpha1-2Galbeta1-3GalNAcbeta1-4(NeuAcalpha2-8NeuAcalpha2-3)Galbeta1-4Glcbeta-Cer; Fucalpha1-2Galbeta1-3GalNAcbeta1-4(NeuAcalpha2-8NeuAcalpha2-3)Galbeta1-4Glcbeta-Cer(d18:1/16:0); Fucalpha1-2Galbeta1-3GalNAcbeta1-4(NeuAcalpha2-8NeuAcalpha2-3)Galbeta1-4Glcbeta-Cer(d18:1/18:0); Fucalpha1-2Galbeta1-3GalNAcbeta1-4(NeuAcalpha2-8NeuAcalpha2-3)Galbeta1-4Glcbeta-Cer(d18:1/20:0); Fucalpha1-2Galbeta1-3GalNAcbeta1-4(NeuAcalpha2-8NeuAcalpha2-3)Galbeta1-4Glcbeta-Cer(d18:1/22:0); Fucalpha1-2Galbeta1-3GalNAcbeta1-4(NeuAcalpha2-8NeuAcalpha2-3)Galbeta1-4Glcbeta-Cer(d18:1/24:0); Fucalpha1-2Galbeta1-3GalNAcbeta1-4(NeuAcalpha2-8NeuAcalpha2-3)Galbeta1-4Glcbeta-Cer(d18:1/24:1(15Z)); Fucalpha1-2Galbeta1-3GalNAcbeta1-4(NeuAcalpha2-8NeuAcalpha2-3)Galbeta1-4Glcbeta-Cer(d18:1/26:0); Fucalpha1-2Galbeta1-3GalNAcbeta1-4(NeuAcalpha2-8NeuAcalpha2-3)Galbeta1-4Glcbeta-Cer(d18:1/26:1(17Z)); Fucalpha1-2Galbeta1-3GalNAcbeta1-4(NeuGcalpha2-3)Galbeta1-4Glcbeta-Cer; Fucalpha1-2Galbeta1-3GalNAcbeta1-4(NeuGcalpha2-3)Galbeta1-4Glcbeta-Cer(d18:1/16:0); Fucalpha1-2Galbeta1-3GalNAcbeta1-4(NeuGcalpha2-3)Galbeta1-4Glcbeta-Cer(d18:1/18:0); Fucalpha1-2Galbeta1-3GalNAcbeta1-4(NeuGcalpha2-3)Galbeta1-4Glcbeta-Cer(d18:1/20:0); Fucalpha1-2Galbeta1-3GalNAcbeta1-4(NeuGcalpha2-3)Galbeta1-4Glcbeta-Cer(d18:1/22:0); Fucalpha1-2Galbeta1-3GalNAcbeta1-4(NeuGcalpha2-3)Galbeta1-4Glcbeta-Cer(d18:1/24:0); Fucalpha1-2Galbeta1-3GalNAcbeta1-4(NeuGcalpha2-3)Galbeta1-4Glcbeta-Cer(d18:1/24:1(15Z)); Fucalpha1-2Galbeta1-3GalNAcbeta1-4(NeuGcalpha2-3)Galbeta1-4Glcbeta-Cer(d18:1/26:0); Fucalpha1-2Galbeta1-3GalNAcbeta1-4(NeuGcalpha2-3)Galbeta1-4Glcbeta-Cer(d18:1/26:1(17Z)); Fucalpha1-2Galbeta1-3GalNAcbeta1-4Galbeta1-4Glcbeta-Cer; Fucalpha1-2Galbeta1-3GalNAcbeta1-4Galbeta1-4Glcbeta-Cer(d18:1/16:0); Fucalpha1-2Galbeta1-3GalNAcbeta1-4Galbeta1-4Glcbeta-Cer(d18:1/18:0); Fucalpha1-2Galbeta1-3GalNAcbeta1-4Galbeta1-4Glcbeta-Cer(d18:1/20:0); Fucalpha1-2Galbeta1-3GalNAcbeta1-4Galbeta1-4Glcbeta-Cer(d18:1/22:0); Fucalpha1-2Galbeta1-3GalNAcbeta1-4Galbeta1-4Glcbeta-Cer(d18:1/24:0); Fucalpha1-2Galbeta1-3GalNAcbeta1-4Galbeta1-4Glcbeta-Cer(d18:1/24:1(15Z)); Fucalpha1-2Galbeta1-3GalNAcbeta1-4Galbeta1-4Glcbeta-Cer(d18:1/26:0); Fucalpha1-2Galbeta1-3GalNAcbeta1-4Galbeta1-4Glcbeta-Cer(d18:1/26:1(17Z)); Fucalpha1-2Galbeta1-3GlcNAcbeta1-3(Fucalpha1-2Galbeta1-3GlcNAcbeta1-6)Galbeta1-3GlcNAcbeta1-3Galbeta1-4Glcbeta-Cer; Fucalpha1-2Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/16:0); Fucalpha1-2Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/18:0); Fucalpha1-2Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/20:0); Fucalpha1-2Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/22:0); Fucalpha1-2Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/24:0); Fucalpha1-2Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/24:1(15Z)); Fucalpha1-2Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/26:0); Fucalpha1-2Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/26:1(17Z)); Fucalpha1-2Galbeta1-3GlcNAcbeta1-3(Fucalpha1-2Galbeta1-4GlcNAcbeta1-6)Galbeta1-3GlcNAcbeta1-3Galbeta1-4Glcbeta-Cer; Fucalpha1-2Galbeta1-3GlcNAcbeta1-3(Fucalpha1-2Galbeta1-4GlcNAcbeta1-6)Galbeta1-3GlcNAcbeta1-3Galbeta1-4Glcbeta-Cer(d18:1/16:0); Fucalpha1-2Galbeta1-3GlcNAcbeta1-3(Fucalpha1-2Galbeta1-4GlcNAcbeta1-6)Galbeta1-3GlcNAcbeta1-3Galbeta1-4Glcbeta-Cer(d18:1/18:0); Fucalpha1-2Galbeta1-3GlcNAcbeta1-3(Fucalpha1-2Galbeta1-4GlcNAcbeta1-6)Galbeta1-3GlcNAcbeta1-3Galbeta1-4Glcbeta-Cer(d18:1/20:0); Fucalpha1-2Galbeta1-3GlcNAcbeta1-3(Fucalpha1-2Galbeta1-4GlcNAcbeta1-6)Galbeta1-3GlcNAcbeta1-3Galbeta1-4Glcbeta-Cer(d18:1/22:0); Fucalpha1-2Galbeta1-3GlcNAcbeta1-3(Fucalpha1-2Galbeta1-4GlcNAcbeta1-6)Galbeta1-3GlcNAcbeta1-3Galbeta1-4Glcbeta-Cer(d18:1/24:0); Fucalpha1-2Galbeta1-3GlcNAcbeta1-3(Fucalpha1-2Galbeta1-4GlcNAcbeta1-6)Galbeta1-3GlcNAcbeta1-3Galbeta1-4Glcbeta-Cer(d18:1/24:1(15Z)); Fucalpha1-2Galbeta1-3GlcNAcbeta1-3(Fucalpha1-2Galbeta1-4GlcNAcbeta1-6)Galbeta1-3GlcNAcbeta1-3Galbeta1-4Glcbeta-Cer(d18:1/26:0); Fucalpha1-2Galbeta1-3GlcNAcbeta1-3(Fucalpha1-2Galbeta1-4GlcNAcbeta1-6)Galbeta1-3GlcNAcbeta1-3Galbeta1-4Glcbeta-Cer(d18:1/26:1(17Z)); Fucalpha1-2Galbeta1-3GlcNAcbeta1-3(Galbeta1-3GlcNAcbeta1-6)Galbeta1-3GlcNAcbeta1-3Galbeta1-4Glcbeta-Cer; Fucalpha1-2Galbeta1-3GlcNAcbeta1-3(Galbeta1-3GlcNAcbeta1-6)Galbeta1-3GlcNAcbeta1-3Galbeta1-4Glcbeta-Cer(d18:1/16:0); Fucalpha1-2Galbeta1-3GlcNAcbeta1-3(Galbeta1-3GlcNAcbeta1-6)Galbeta1-3GlcNAcbeta1-3Galbeta1-4Glcbeta-Cer(d18:1/18:0); Fucalpha1-2Galbeta1-3GlcNAcbeta1-3(Galbeta1-3GlcNAcbeta1-6)Galbeta1-3GlcNAcbeta1-3Galbeta1-4Glcbeta-Cer(d18:1/20:0); Fucalpha1-2Galbeta1-3GlcNAcbeta1-3(Galbeta1-3GlcNAcbeta1-6)Galbeta1-3GlcNAcbeta1-3Galbeta1-4Glcbeta-Cer(d18:1/22:0); Fucalpha1-2Galbeta1-3GlcNAcbeta1-3(Galbeta1-3GlcNAcbeta1-6)Galbeta1-3GlcNAcbeta1-3Galbeta1-4Glcbeta-Cer(d18:1/24:0); Fucalpha1-2Galbeta1-3GlcNAcbeta1-3(Galbeta1-3GlcNAcbeta1-6)Galbeta1-3GlcNAcbeta1-3Galbeta1-4Glcbeta-Cer(d18:1/24:1(15Z)); Fucalpha1-2Galbeta1-3GlcNAcbeta1-3(Galbeta1-3GlcNAcbeta1-6)Galbeta1-3GlcNAcbeta1-3Galbeta1-4Glcbeta-Cer(d18:1/26:0); Fucalpha1-2Galbeta1-3GlcNAcbeta1-3(Galbeta1-3GlcNAcbeta1-6)Galbeta1-3GlcNAcbeta1-3Galbeta1-4Glcbeta-Cer(d18:1/26:1(17Z)); Fucalpha1-2Galbeta1-3GlcNAcbeta1-3(Galbeta1-4GlcNAcbeta1-6)Galbeta1-3GlcNAcbeta1-3Galbeta1-4Glcbeta-Cer; Fucalpha1-2Galbeta1-3GlcNAcbeta1-3(Galbeta1-4GlcNAcbeta1-6)Galbeta1-3GlcNAcbeta1-3Galbeta1-4Glcbeta-Cer(d18:1/16:0); Fucalpha1-2Galbeta1-3GlcNAcbeta1-3(Galbeta1-4GlcNAcbeta1-6)Galbeta1-3GlcNAcbeta1-3Galbeta1-4Glcbeta-Cer(d18:1/18:0); Fucalpha1-2Galbeta1-3GlcNAcbeta1-3(Galbeta1-4GlcNAcbeta1-6)Galbeta1-3GlcNAcbeta1-3Galbeta1-4Glcbeta-Cer(d18:1/20:0); Fucalpha1-2Galbeta1-3GlcNAcbeta1-3(Galbeta1-4GlcNAcbeta1-6)Galbeta1-3GlcNAcbeta1-3Galbeta1-4Glcbeta-Cer(d18:1/22:0); Fucalpha1-2Galbeta1-3GlcNAcbeta1-3(Galbeta1-4GlcNAcbeta1-6)Galbeta1-3GlcNAcbeta1-3Galbeta1-4Glcbeta-Cer(d18:1/24:0); Fucalpha1-2Galbeta1-3GlcNAcbeta1-3(Galbeta1-4GlcNAcbeta1-6)Galbeta1-3GlcNAcbeta1-3Galbeta1-4Glcbeta-Cer(d18:1/24:1(15Z)); Fucalpha1-2Galbeta1-3GlcNAcbeta1-3(Galbeta1-4GlcNAcbeta1-6)Galbeta1-3GlcNAcbeta1-3Galbeta1-4Glcbeta-Cer(d18:1/26:0); Fucalpha1-2Galbeta1-3GlcNAcbeta1-3(Galbeta1-4GlcNAcbeta1-6)Galbeta1-3GlcNAcbeta1-3Galbeta1-4Glcbeta-Cer(d18:1/26:1(17Z)); Fucalpha1-2Galbeta1-3GlcNAcbeta1-3(GlcNAcbeta1-6)Galbeta1-3GlcNAcbeta1-3Galbeta1-4Glcbeta-Cer; Fucalpha1-2Galbeta1-3GlcNAcbeta1-3(GlcNAcbeta1-6)Galbeta1-3GlcNAcbeta1-3Galbeta1-4Glcbeta-Cer(d18:1/16:0); Fucalpha1-2Galbeta1-3GlcNAcbeta1-3(GlcNAcbeta1-6)Galbeta1-3GlcNAcbeta1-3Galbeta1-4Glcbeta-Cer(d18:1/18:0); Fucalpha1-2Galbeta1-3GlcNAcbeta1-3(GlcNAcbeta1-6)Galbeta1-3GlcNAcbeta1-3Galbeta1-4Glcbeta-Cer(d18:1/20:0); Fucalpha1-2Galbeta1-3GlcNAcbeta1-3(GlcNAcbeta1-6)Galbeta1-3GlcNAcbeta1-3Galbeta1-4Glcbeta-Cer(d18:1/22:0); Fucalpha1-2Galbeta1-3GlcNAcbeta1-3(GlcNAcbeta1-6)Galbeta1-3GlcNAcbeta1-3Galbeta1-4Glcbeta-Cer(d18:1/24:0); Fucalpha1-2Galbeta1-3GlcNAcbeta1-3(GlcNAcbeta1-6)Galbeta1-3GlcNAcbeta1-3Galbeta1-4Glcbeta-Cer(d18:1/24:1(15Z)); Fucalpha1-2Galbeta1-3GlcNAcbeta1-3(GlcNAcbeta1-6)Galbeta1-3GlcNAcbeta1-3Galbeta1-4Glcbeta-Cer(d18:1/26:0); Fucalpha1-2Galbeta1-3GlcNAcbeta1-3(GlcNAcbeta1-6)Galbeta1-3GlcNAcbeta1-3Galbeta1-4Glcbeta-Cer(d18:1/26:1(17Z)); Fucalpha1-2Galbeta1-3GlcNAcbeta1-3Galbeta1-3GlcNAcbeta1-3Galbeta1-4Glcbeta-Cer; Fucalpha1-2Galbeta1-3GlcNAcbeta1-3Galbeta1-3GlcNAcbeta1-3Galbeta1-4Glcbeta-Cer(d18:1/16:0); Fucalpha1-2Galbeta1-3GlcNAcbeta1-3Galbeta1-3GlcNAcbeta1-3Galbeta1-4Glcbeta-Cer(d18:1/18:0); Fucalpha1-2Galbeta1-3GlcNAcbeta1-3Galbeta1-3GlcNAcbeta1-3Galbeta1-4Glcbeta-Cer(d18:1/20:0); Fucalpha1-2Galbeta1-3GlcNAcbeta1-3Galbeta1-3GlcNAcbeta1-3Galbeta1-4Glcbeta-Cer(d18:1/22:0); Fucalpha1-2Galbeta1-3GlcNAcbeta1-3Galbeta1-3GlcNAcbeta1-3Galbeta1-4Glcbeta-Cer(d18:1/24:0); Fucalpha1-2Galbeta1-3GlcNAcbeta1-3Galbeta1-3GlcNAcbeta1-3Galbeta1-4Glcbeta-Cer(d18:1/24:1(15Z)); Fucalpha1-2Galbeta1-3GlcNAcbeta1-3Galbeta1-3GlcNAcbeta1-3Galbeta1-4Glcbeta-Cer(d18:1/26:0); Fucalpha1-2Galbeta1-3GlcNAcbeta1-3Galbeta1-3GlcNAcbeta1-3Galbeta1-4Glcbeta-Cer(d18:1/26:1(17Z)); Fucalpha1-2Galbeta1-3GlcNAcbeta1-3Galbeta1-4Glcbeta-Cer; Fucalpha1-2Galbeta1-3GlcNAcbeta1-3Galbeta1-4Glcbeta-Cer(d18:1/16:0); Fucalpha1-2Galbeta1-3GlcNAcbeta1-3Galbeta1-4Glcbeta-Cer(d18:1/18:0); Fucalpha1-2Galbeta1-3GlcNAcbeta1-3Galbeta1-4Glcbeta-Cer(d18:1/20:0); Fucalpha1-2Galbeta1-3GlcNAcbeta1-3Galbeta1-4Glcbeta-Cer(d18:1/22:0); Fucalpha1-2Galbeta1-3GlcNAcbeta1-3Galbeta1-4Glcbeta-Cer(d18:1/24:0); Fucalpha1-2Galbeta1-3GlcNAcbeta1-3Galbeta1-4Glcbeta-Cer(d18:1/24:1(15Z)); Fucalpha1-2Galbeta1-3GlcNAcbeta1-3Galbeta1-4Glcbeta-Cer(d18:1/26:0); Fucalpha1-2Galbeta1-3GlcNAcbeta1-3Galbeta1-4Glcbeta-Cer(d18:1/26:1(17Z)); Fucalpha1-2Galbeta1-4(Fucalpha1-3)GlcNAcbeta1-3Galbeta1-3(Fucalpha1-4)GlcNAcbeta1-3Galbeta1-4Glcbeta-Cer; Fucalpha1-2Galbeta1-4(Fucalpha1-3)GlcNAcbeta1-3Galbeta1-3(Fucalpha1-4)GlcNAcbeta1-3Galbeta1-4Glcbeta-Cer(d18:1/16:0); Fucalpha1-2Galbeta1-4(Fucalpha1-3)GlcNAcbeta1-3Galbeta1-3(Fucalpha1-4)GlcNAcbeta1-3Galbeta1-4Glcbeta-Cer(d18:1/18:0); Fucalpha1-2Galbeta1-4(Fucalpha1-3)GlcNAcbeta1-3Galbeta1-3(Fucalpha1-4)GlcNAcbeta1-3Galbeta1-4Glcbeta-Cer(d18:1/20:0); Fucalpha1-2Galbeta1-4(Fucalpha1-3)GlcNAcbeta1-3Galbeta1-3(Fucalpha1-4)GlcNAcbeta1-3Galbeta1-4Glcbeta-Cer(d18:1/22:0); Fucalpha1-2Galbeta1-4(Fucalpha1-3)GlcNAcbeta1-3Galbeta1-3(Fucalpha1-4)GlcNAcbeta1-3Galbeta1-4Glcbeta-Cer(d18:1/24:0); Fucalpha1-2Galbeta1-4(Fucalpha1-3)GlcNAcbeta1-3Galbeta1-3(Fucalpha1-4)GlcNAcbeta1-3Galbeta1-4Glcbeta-Cer(d18:1/24:1(15Z)); Fucalpha1-2Galbeta1-4(Fucalpha1-3)GlcNAcbeta1-3Galbeta1-3(Fucalpha1-4)GlcNAcbeta1-3Galbeta1-4Glcbeta-Cer(d18:1/26:0); Fucalpha1-2Galbeta1-4(Fucalpha1-3)GlcNAcbeta1-3Galbeta1-3(Fucalpha1-4)GlcNAcbeta1-3Galbeta1-4Glcbeta-Cer(d18:1/26:1(17Z)); Fucalpha1-2Galbeta1-4(Fucalpha1-3)GlcNAcbeta1-3Galbeta1-3GlcNAcbeta1-3Galbeta1-4Glcbeta-Cer; Fucalpha1-2Galbeta1-4(Fucalpha1-3)GlcNAcbeta1-3Galbeta1-3GlcNAcbeta1-3Galbeta1-4Glcbeta-Cer(d18:1/16:0); Fucalpha1-2Galbeta1-4(Fucalpha1-3)GlcNAcbeta1-3Galbeta1-3GlcNAcbeta1-3Galbeta1-4Glcbeta-Cer(d18:1/18:0); Fucalpha1-2Galbeta1-4(Fucalpha1-3)GlcNAcbeta1-3Galbeta1-3GlcNAcbeta1-3Galbeta1-4Glcbeta-Cer(d18:1/20:0); Fucalpha1-2Galbeta1-4(Fucalpha1-3)GlcNAcbeta1-3Galbeta1-3GlcNAcbeta1-3Galbeta1-4Glcbeta-Cer(d18:1/22:0); Fucalpha1-2Galbeta1-4(Fucalpha1-3)GlcNAcbeta1-3Galbeta1-3GlcNAcbeta1-3Galbeta1-4Glcbeta-Cer(d18:1/24:0); Fucalpha1-2Galbeta1-4(Fucalpha1-3)GlcNAcbeta1-3Galbeta1-3GlcNAcbeta1-3Galbeta1-4Glcbeta-Cer(d18:1/24:1(15Z)); Fucalpha1-2Galbeta1-4(Fucalpha1-3)GlcNAcbeta1-3Galbeta1-3GlcNAcbeta1-3Galbeta1-4Glcbeta-Cer(d18:1/26:0); Fucalpha1-2Galbeta1-4(Fucalpha1-3)GlcNAcbeta1-3Galbeta1-3GlcNAcbeta1-3Galbeta1-4Glcbeta-Cer(d18:1/26:1(17Z)); Fucalpha1-2Galbeta1-4(Fucalpha1-3)GlcNAcbeta1-3Galbeta1-4(Fucalpha1-3)GlcNAcbeta1-3Galbeta1-4Glcbeta-Cer; Fucalpha1-2Galbeta1-4(Fucalpha1-3)GlcNAcbeta1-3Galbeta1-4(Fucalpha1-3)GlcNAcbeta1-3Galbeta1-4Glcbeta-Cer(d18:1/16:0); Fucalpha1-2Galbeta1-4(Fucalpha1-3)GlcNAcbeta1-3Galbeta1-4(Fucalpha1-3)GlcNAcbeta1-3Galbeta1-4Glcbeta-Cer(d18:1/18:0); Fucalpha1-2Galbeta1-4(Fucalpha1-3)GlcNAcbeta1-3Galbeta1-4(Fucalpha1-3)GlcNAcbeta1-3Galbeta1-4Glcbeta-Cer(d18:1/20:0); Fucalpha1-2Galbeta1-4(Fucalpha1-3)GlcNAcbeta1-3Galbeta1-4(Fucalpha1-3)GlcNAcbeta1-3Galbeta1-4Glcbeta-Cer(d18:1/22:0); Fucalpha1-2Galbeta1-4(Fucalpha1-3)GlcNAcbeta1-3Galbeta1-4(Fucalpha1-3)GlcNAcbeta1-3Galbeta1-4Glcbeta-Cer(d18:1/24:0); Fucalpha1-2Galbeta1-4(Fucalpha1-3)GlcNAcbeta1-3Galbeta1-4(Fucalpha1-3)GlcNAcbeta1-3Galbeta1-4Glcbeta-Cer(d18:1/24:1(15Z)); Fucalpha1-2Galbeta1-4(Fucalpha1-3)GlcNAcbeta1-3Galbeta1-4(Fucalpha1-3)GlcNAcbeta1-3Galbeta1-4Glcbeta-Cer(d18:1/26:0); Fucalpha1-2Galbeta1-4(Fucalpha1-3)GlcNAcbeta1-3Galbeta1-4(Fucalpha1-3)GlcNAcbeta1-3Galbeta1-4Glcbeta-Cer(d18:1/26:1(17Z)); Fucalpha1-2Galbeta1-4(Fucalpha1-3)GlcNAcbeta1-3Galbeta1-4Glcbeta-Cer; Fucalpha1-2Galbeta1-4(Fucalpha1-3)GlcNAcbeta1-3Galbeta1-4Glcbeta-Cer(d18:1/16:0); Fucalpha1-2Galbeta1-4(Fucalpha1-3)GlcNAcbeta1-3Galbeta1-4Glcbeta-Cer(d18:1/18:0); Fucalpha1-2Galbeta1-4(Fucalpha1-3)GlcNAcbeta1-3Galbeta1-4Glcbeta-Cer(d18:1/20:0); Fucalpha1-2Galbeta1-4(Fucalpha1-3)GlcNAcbeta1-3Galbeta1-4Glcbeta-Cer(d18:1/22:0); Fucalpha1-2Galbeta1-4(Fucalpha1-3)GlcNAcbeta1-3Galbeta1-4Glcbeta-Cer(d18:1/24:0); Fucalpha1-2Galbeta1-4(Fucalpha1-3)GlcNAcbeta1-3Galbeta1-4Glcbeta-Cer(d18:1/24:1(15Z)); Fucalpha1-2Galbeta1-4(Fucalpha1-3)GlcNAcbeta1-3Galbeta1-4Glcbeta-Cer(d18:1/26:0); Fucalpha1-2Galbeta1-4(Fucalpha1-3)GlcNAcbeta1-3Galbeta1-4Glcbeta-Cer(d18:1/26:1(17Z)); Fucalpha1-2Galbeta1-4(Fucalpha1-3)GlcNAcbeta1-3Galbeta1-4GlcNAcbeta1-3Galbeta1-4Glcbeta-Cer; Fucalpha1-2Galbeta1-4(Fucalpha1-3)GlcNAcbeta1-3Galbeta1-4GlcNAcbeta1-3Galbeta1-4Glcbeta-Cer(d18:1/16:0); Fucalpha1-2Galbeta1-4(Fucalpha1-3)GlcNAcbeta1-3Galbeta1-4GlcNAcbeta1-3Galbeta1-4Glcbeta-Cer(d18:1/18:0); Fucalpha1-2Galbeta1-4(Fucalpha1-3)GlcNAcbeta1-3Galbeta1-4GlcNAcbeta1-3Galbeta1-4Glcbeta-Cer(d18:1/20:0); Fucalpha1-2Galbeta1-4(Fucalpha1-3)GlcNAcbeta1-3Galbeta1-4GlcNAcbeta1-3Galbeta1-4Glcbeta-Cer(d18:1/22:0); Fucalpha1-2Galbeta1-4(Fucalpha1-3)GlcNAcbeta1-3Galbeta1-4GlcNAcbeta1-3Galbeta1-4Glcbeta-Cer(d18:1/24:0); Fucalpha1-2Galbeta1-4(Fucalpha1-3)GlcNAcbeta1-3Galbeta1-4GlcNAcbeta1-3Galbeta1-4Glcbeta-Cer(d18:1/24:1(15Z)); Fucalpha1-2Galbeta1-4(Fucalpha1-3)GlcNAcbeta1-3Galbeta1-4GlcNAcbeta1-3Galbeta1-4Glcbeta-Cer(d18:1/26:0); Fucalpha1-2Galbeta1-4(Fucalpha1-3)GlcNAcbeta1-3Galbeta1-4GlcNAcbeta1-3Galbeta1-4Glcbeta-Cer(d18:1/26:1(17Z)); Fucalpha1-2Galbeta1-4GlcNAcbeta1-3(Fucalpha1-2(Galalpha1-3)Galbeta1-4GlcNAcbeta1-6)Galbeta1-4GlcNAcbeta1-3Galbeta1-4Glcbeta-Cer; Fucalpha1-2Galbeta1-4GlcNAcbeta1-3(Fucalpha1-2(Galalpha1-3)Galbeta1-4GlcNAcbeta1-6)Galbeta1-4GlcNAcbeta1-3Galbeta1-4Glcbeta-Cer(d18:1/16:0); Fucalpha1-2Galbeta1-4GlcNAcbeta1-3(Fucalpha1-2(Galalpha1-3)Galbeta1-4GlcNAcbeta1-6)Galbeta1-4GlcNAcbeta1-3Galbeta1-4Glcbeta-Cer(d18:1/18:0); Fucalpha1-2Galbeta1-4GlcNAcbeta1-3(Fucalpha1-2(Galalpha1-3)Galbeta1-4GlcNAcbeta1-6)Galbeta1-4GlcNAcbeta1-3Galbeta1-4Glcbeta-Cer(d18:1/20:0); Fucalpha1-2Galbeta1-4GlcNAcbeta1-3(Fucalpha1-2(Galalpha1-3)Galbeta1-4GlcNAcbeta1-6)Galbeta1-4GlcNAcbeta1-3Galbeta1-4Glcbeta-Cer(d18:1/22:0); Fucalpha1-2Galbeta1-4GlcNAcbeta1-3(Fucalpha1-2(Galalpha1-3)Galbeta1-4GlcNAcbeta1-6)Galbeta1-4GlcNAcbeta1-3Galbeta1-4Glcbeta-Cer(d18:1/24:0); Fucalpha1-2Galbeta1-4GlcNAcbeta1-3(Fucalpha1-2(Galalpha1-3)Galbeta1-4GlcNAcbeta1-6)Galbeta1-4GlcNAcbeta1-3Galbeta1-4Glcbeta-Cer(d18:1/24:1(15Z)); Fucalpha1-2Galbeta1-4GlcNAcbeta1-3(Fucalpha1-2(Galalpha1-3)Galbeta1-4GlcNAcbeta1-6)Galbeta1-4GlcNAcbeta1-3Galbeta1-4Glcbeta-Cer(d18:1/26:0); Fucalpha1-2Galbeta1-4GlcNAcbeta1-3(Fucalpha1-2(Galalpha1-3)Galbeta1-4GlcNAcbeta1-6)Galbeta1-4GlcNAcbeta1-3Galbeta1-4Glcbeta-Cer(d18:1/26:1(17Z)); Fucalpha1-2Galbeta1-4GlcNAcbeta1-3(Fucalpha1-2Galbeta1-4GlcNAcbeta1-6)Galbeta1-4GlcNAcbeta1-3Galbeta1-4Glcbeta-Cer; Fucalpha1-2Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/16:0); Fucalpha1-2Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/18:0); Fucalpha1-2Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/20:0); Fucalpha1-2Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/22:0); Fucalpha1-2Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/24:0); Fucalpha1-2Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/24:1(15Z)); Fucalpha1-2Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/26:0); Fucalpha1-2Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/26:1(17Z)); Fucalpha1-2Galbeta1-4GlcNAcbeta1-3(Galbeta1-4GlcNAcbeta1-6)Galbeta1-4GlcNAcbeta1-3Galbeta1-4Glcbeta-Cer; Fucalpha1-2Galbeta1-4GlcNAcbeta1-3(Galbeta1-4GlcNAcbeta1-6)Galbeta1-4GlcNAcbeta1-3Galbeta1-4Glcbeta-Cer(d18:1/16:0); Fucalpha1-2Galbeta1-4GlcNAcbeta1-3(Galbeta1-4GlcNAcbeta1-6)Galbeta1-4GlcNAcbeta1-3Galbeta1-4Glcbeta-Cer(d18:1/18:0); Fucalpha1-2Galbeta1-4GlcNAcbeta1-3(Galbeta1-4GlcNAcbeta1-6)Galbeta1-4GlcNAcbeta1-3Galbeta1-4Glcbeta-Cer(d18:1/20:0); Fucalpha1-2Galbeta1-4GlcNAcbeta1-3(Galbeta1-4GlcNAcbeta1-6)Galbeta1-4GlcNAcbeta1-3Galbeta1-4Glcbeta-Cer(d18:1/22:0); Fucalpha1-2Galbeta1-4GlcNAcbeta1-3(Galbeta1-4GlcNAcbeta1-6)Galbeta1-4GlcNAcbeta1-3Galbeta1-4Glcbeta-Cer(d18:1/24:0); Fucalpha1-2Galbeta1-4GlcNAcbeta1-3(Galbeta1-4GlcNAcbeta1-6)Galbeta1-4GlcNAcbeta1-3Galbeta1-4Glcbeta-Cer(d18:1/24:1(15Z)); Fucalpha1-2Galbeta1-4GlcNAcbeta1-3(Galbeta1-4GlcNAcbeta1-6)Galbeta1-4GlcNAcbeta1-3Galbeta1-4Glcbeta-Cer(d18:1/26:0); Fucalpha1-2Galbeta1-4GlcNAcbeta1-3(Galbeta1-4GlcNAcbeta1-6)Galbeta1-4GlcNAcbeta1-3Galbeta1-4Glcbeta-Cer(d18:1/26:1(17Z)); Fucalpha1-2Galbeta1-4GlcNAcbeta1-3(GalNAcalpha1-3(Fucalpha1-2)Galbeta1-4GlcNAcbeta1-6)Galbeta1-4GlcNAcbeta1-3Galbeta1-4Glcbeta-Cer; Fucalpha1-2Galbeta1-4GlcNAcbeta1-3(GalNAcalpha1-3(Fucalpha1-2)Galbeta1-4GlcNAcbeta1-6)Galbeta1-4GlcNAcbeta1-3Galbeta1-4Glcbeta-Cer(d18:1/16:0); Fucalpha1-2Galbeta1-4GlcNAcbeta1-3(GalNAcalpha1-3(Fucalpha1-2)Galbeta1-4GlcNAcbeta1-6)Galbeta1-4GlcNAcbeta1-3Galbeta1-4Glcbeta-Cer(d18:1/18:0); Fucalpha1-2Galbeta1-4GlcNAcbeta1-3(GalNAcalpha1-3(Fucalpha1-2)Galbeta1-4GlcNAcbeta1-6)Galbeta1-4GlcNAcbeta1-3Galbeta1-4Glcbeta-Cer(d18:1/20:0); Fucalpha1-2Galbeta1-4GlcNAcbeta1-3(GalNAcalpha1-3(Fucalpha1-2)Galbeta1-4GlcNAcbeta1-6)Galbeta1-4GlcNAcbeta1-3Galbeta1-4Glcbeta-Cer(d18:1/22:0); Fucalpha1-2Galbeta1-4GlcNAcbeta1-3(GalNAcalpha1-3(Fucalpha1-2)Galbeta1-4GlcNAcbeta1-6)Galbeta1-4GlcNAcbeta1-3Galbeta1-4Glcbeta-Cer(d18:1/24:0); Fucalpha1-2Galbeta1-4GlcNAcbeta1-3(GalNAcalpha1-3(Fucalpha1-2)Galbeta1-4GlcNAcbeta1-6)Galbeta1-4GlcNAcbeta1-3Galbeta1-4Glcbeta-Cer(d18:1/24:1(15Z)); Fucalpha1-2Galbeta1-4GlcNAcbeta1-3(GalNAcalpha1-3(Fucalpha1-2)Galbeta1-4GlcNAcbeta1-6)Galbeta1-4GlcNAcbeta1-3Galbeta1-4Glcbeta-Cer(d18:1/26:0); Fucalpha1-2Galbeta1-4GlcNAcbeta1-3(GalNAcalpha1-3(Fucalpha1-2)Galbeta1-4GlcNAcbeta1-6)Galbeta1-4GlcNAcbeta1-3Galbeta1-4Glcbeta-Cer(d18:1/26:1(17Z)); Fucalpha1-2Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer; Fucalpha1-2Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/16:0); Fucalpha1-2Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/18:0); Fucalpha1-2Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/20:0); Fucalpha1-2Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/22:0); Fucalpha1-2Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/24:0); Fucalpha1-2Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/24:1(15Z)); Fucalpha1-2Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/26:0); Fucalpha1-2Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/26:1(17Z)); Fucalpha1-2Galbeta1-4GlcNAcbeta1-3Galbeta1-3(Fucalpha1-4)GlcNAcbeta1-3Galbeta1-4Glcbeta-Cer; Fucalpha1-2Galbeta1-4GlcNAcbeta1-3Galbeta1-3(Fucalpha1-4)GlcNAcbeta1-3Galbeta1-4Glcbeta-Cer(d18:1/16:0); Fucalpha1-2Galbeta1-4GlcNAcbeta1-3Galbeta1-3(Fucalpha1-4)GlcNAcbeta1-3Galbeta1-4Glcbeta-Cer(d18:1/18:0); Fucalpha1-2Galbeta1-4GlcNAcbeta1-3Galbeta1-3(Fucalpha1-4)GlcNAcbeta1-3Galbeta1-4Glcbeta-Cer(d18:1/20:0); Fucalpha1-2Galbeta1-4GlcNAcbeta1-3Galbeta1-3(Fucalpha1-4)GlcNAcbeta1-3Galbeta1-4Glcbeta-Cer(d18:1/22:0); Fucalpha1-2Galbeta1-4GlcNAcbeta1-3Galbeta1-3(Fucalpha1-4)GlcNAcbeta1-3Galbeta1-4Glcbeta-Cer(d18:1/24:0); Fucalpha1-2Galbeta1-4GlcNAcbeta1-3Galbeta1-3(Fucalpha1-4)GlcNAcbeta1-3Galbeta1-4Glcbeta-Cer(d18:1/24:1(15Z)); Fucalpha1-2Galbeta1-4GlcNAcbeta1-3Galbeta1-3(Fucalpha1-4)GlcNAcbeta1-3Galbeta1-4Glcbeta-Cer(d18:1/26:0); Fucalpha1-2Galbeta1-4GlcNAcbeta1-3Galbeta1-3(Fucalpha1-4)GlcNAcbeta1-3Galbeta1-4Glcbeta-Cer(d18:1/26:1(17Z)); Fucalpha1-2Galbeta1-4GlcNAcbeta1-3Galbeta1-3GlcNAcbeta1-3Galbeta1-4Glcbeta-Cer; Fucalpha1-2Galbeta1-4GlcNAcbeta1-3Galbeta1-3GlcNAcbeta1-3Galbeta1-4Glcbeta-Cer(d18:1/16:0); Fucalpha1-2Galbeta1-4GlcNAcbeta1-3Galbeta1-3GlcNAcbeta1-3Galbeta1-4Glcbeta-Cer(d18:1/18:0); Fucalpha1-2Galbeta1-4GlcNAcbeta1-3Galbeta1-3GlcNAcbeta1-3Galbeta1-4Glcbeta-Cer(d18:1/20:0); Fucalpha1-2Galbeta1-4GlcNAcbeta1-3Galbeta1-3GlcNAcbeta1-3Galbeta1-4Glcbeta-Cer(d18:1/22:0); Fucalpha1-2Galbeta1-4GlcNAcbeta1-3Galbeta1-3GlcNAcbeta1-3Galbeta1-4Glcbeta-Cer(d18:1/24:0); Fucalpha1-2Galbeta1-4GlcNAcbeta1-3Galbeta1-3GlcNAcbeta1-3Galbeta1-4Glcbeta-Cer(d18:1/24:1(15Z)); Fucalpha1-2Galbeta1-4GlcNAcbeta1-3Galbeta1-3GlcNAcbeta1-3Galbeta1-4Glcbeta-Cer(d18:1/26:0); Fucalpha1-2Galbeta1-4GlcNAcbeta1-3Galbeta1-3GlcNAcbeta1-3Galbeta1-4Glcbeta-Cer(d18:1/26:1(17Z)); Fucalpha1-2Galbeta1-4GlcNAcbeta1-3Galbeta1-4Glcbeta-Cer; Fucalpha1-2Galbeta1-4GlcNAcbeta1-3Galbeta1-4Glcbeta-Cer(d18:1/16:0); Fucalpha1-2Galbeta1-4GlcNAcbeta1-3Galbeta1-4Glcbeta-Cer(d18:1/18:0); Fucalpha1-2Galbeta1-4GlcNAcbeta1-3Galbeta1-4Glcbeta-Cer(d18:1/20:0); Fucalpha1-2Galbeta1-4GlcNAcbeta1-3Galbeta1-4Glcbeta-Cer(d18:1/22:0); Fucalpha1-2Galbeta1-4GlcNAcbeta1-3Galbeta1-4Glcbeta-Cer(d18:1/24:0); Fucalpha1-2Galbeta1-4GlcNAcbeta1-3Galbeta1-4Glcbeta-Cer(d18:1/24:1(15Z)); Fucalpha1-2Galbeta1-4GlcNAcbeta1-3Galbeta1-4Glcbeta-Cer(d18:1/26:0); Fucalpha1-2Galbeta1-4GlcNAcbeta1-3Galbeta1-4Glcbeta-Cer(d18:1/26:1(17Z)); Fucalpha1-2Galbeta1-4GlcNAcbeta1-3Galbeta1-4GlcNAcbeta1-3(Galbeta1-4GlcNAcbeta1-6)Galbeta1-4GlcNAcbeta1-3Galbeta1-4Glcbeta-Cer; Fucalpha1-2Galbeta1-4GlcNAcbeta1-3Galbeta1-4GlcNAcbeta1-3(Galbeta1-4GlcNAcbeta1-6)Galbeta1-4GlcNAcbeta1-3Galbeta1-4Glcbeta-Cer(d18:1/16:0); Fucalpha1-2Galbeta1-4GlcNAcbeta1-3Galbeta1-4GlcNAcbeta1-3(Galbeta1-4GlcNAcbeta1-6)Galbeta1-4GlcNAcbeta1-3Galbeta1-4Glcbeta-Cer(d18:1/18:0); Fucalpha1-2Galbeta1-4GlcNAcbeta1-3Galbeta1-4GlcNAcbeta1-3(Galbeta1-4GlcNAcbeta1-6)Galbeta1-4GlcNAcbeta1-3Galbeta1-4Glcbeta-Cer(d18:1/20:0); Fucalpha1-2Galbeta1-4GlcNAcbeta1-3Galbeta1-4GlcNAcbeta1-3(Galbeta1-4GlcNAcbeta1-6)Galbeta1-4GlcNAcbeta1-3Galbeta1-4Glcbeta-Cer(d18:1/22:0); Fucalpha1-2Galbeta1-4GlcNAcbeta1-3Galbeta1-4GlcNAcbeta1-3(Galbeta1-4GlcNAcbeta1-6)Galbeta1-4GlcNAcbeta1-3Galbeta1-4Glcbeta-Cer(d18:1/24:0); Fucalpha1-2Galbeta1-4GlcNAcbeta1-3Galbeta1-4GlcNAcbeta1-3(Galbeta1-4GlcNAcbeta1-6)Galbeta1-4GlcNAcbeta1-3Galbeta1-4Glcbeta-Cer(d18:1/24:1(15Z)); Fucalpha1-2Galbeta1-4GlcNAcbeta1-3Galbeta1-4GlcNAcbeta1-3(Galbeta1-4GlcNAcbeta1-6)Galbeta1-4GlcNAcbeta1-3Galbeta1-4Glcbeta-Cer(d18:1/26:0); Fucalpha1-2Galbeta1-4GlcNAcbeta1-3Galbeta1-4GlcNAcbeta1-3(Galbeta1-4GlcNAcbeta1-6)Galbeta1-4GlcNAcbeta1-3Galbeta1-4Glcbeta-Cer(d18:1/26:1(17Z)); Fucalpha1-2Galbeta1-4GlcNAcbeta1-3Galbeta1-4GlcNAcbeta1-3Galbeta1-4Glcbeta-Cer; Fucalpha1-2Galbeta1-4GlcNAcbeta1-3Galbeta1-4GlcNAcbeta1-3Galbeta1-4Glcbeta-Cer(d18:1/16:0); Fucalpha1-2Galbeta1-4GlcNAcbeta1-3Galbeta1-4GlcNAcbeta1-3Galbeta1-4Glcbeta-Cer(d18:1/18:0); Fucalpha1-2Galbeta1-4GlcNAcbeta1-3Galbeta1-4GlcNAcbeta1-3Galbeta1-4Glcbeta-Cer(d18:1/20:0); Fucalpha1-2Galbeta1-4GlcNAcbeta1-3Galbeta1-4GlcNAcbeta1-3Galbeta1-4Glcbeta-Cer(d18:1/22:0); Fucalpha1-2Galbeta1-4GlcNAcbeta1-3Galbeta1-4GlcNAcbeta1-3Galbeta1-4Glcbeta-Cer(d18:1/24:0); Fucalpha1-2Galbeta1-4GlcNAcbeta1-3Galbeta1-4GlcNAcbeta1-3Galbeta1-4Glcbeta-Cer(d18:1/24:1(15Z)); Fucalpha1-2Galbeta1-4GlcNAcbeta1-3Galbeta1-4GlcNAcbeta1-3Galbeta1-4Glcbeta-Cer(d18:1/26:0); Fucalpha1-2Galbeta1-4GlcNAcbeta1-3Galbeta1-4GlcNAcbeta1-3Galbeta1-4Glcbeta-Cer(d18:1/26:1(17Z)); Fucalpha1-2Galbeta1-4GlcNAcbeta1-3Galbeta1-4GlcNAcbeta1-3Galbeta1-4GlcNAcbeta1-3Galbeta1-4Glcbeta-Cer; Fucalpha1-2Galbeta1-4GlcNAcbeta1-3Galbeta1-4GlcNAcbeta1-3Galbeta1-4GlcNAcbeta1-3Galbeta1-4Glcbeta-Cer(d18:1/16:0); Fucalpha1-2Galbeta1-4GlcNAcbeta1-3Galbeta1-4GlcNAcbeta1-3Galbeta1-4GlcNAcbeta1-3Galbeta1-4Glcbeta-Cer(d18:1/18:0); Fucalpha1-2Galbeta1-4GlcNAcbeta1-3Galbeta1-4GlcNAcbeta1-3Galbeta1-4GlcNAcbeta1-3Galbeta1-4Glcbeta-Cer(d18:1/20:0); Fucalpha1-2Galbeta1-4GlcNAcbeta1-3Galbeta1-4GlcNAcbeta1-3Galbeta1-4GlcNAcbeta1-3Galbeta1-4Glcbeta-Cer(d18:1/22:0); Fucalpha1-2Galbeta1-4GlcNAcbeta1-3Galbeta1-4GlcNAcbeta1-3Galbeta1-4GlcNAcbeta1-3Galbeta1-4Glcbeta-Cer(d18:1/24:0); Fucalpha1-2Galbeta1-4GlcNAcbeta1-3Galbeta1-4GlcNAcbeta1-3Galbeta1-4GlcNAcbeta1-3Galbeta1-4Glcbeta-Cer(d18:1/24:1(15Z)); Fucalpha1-2Galbeta1-4GlcNAcbeta1-3Galbeta1-4GlcNAcbeta1-3Galbeta1-4GlcNAcbeta1-3Galbeta1-4Glcbeta-Cer(d18:1/26:0); Fucalpha1-2Galbeta1-4GlcNAcbeta1-3Galbeta1-4GlcNAcbeta1-3Galbeta1-4GlcNAcbeta1-3Galbeta1-4Glcbeta-Cer(d18:1/26:1(17Z)); Fucalpha1-2GalNAcbeta1-4(NeuAcalpha2-8NeuAcalpha2-3)Galbeta1-4Glcbeta-Cer; Fucalpha1-2GalNAcbeta1-4(NeuAcalpha2-8NeuAcalpha2-3)Galbeta1-4Glcbeta-Cer(d18:1/16:0); Fucalpha1-2GalNAcbeta1-4(NeuAcalpha2-8NeuAcalpha2-3)Galbeta1-4Glcbeta-Cer(d18:1/18:0); Fucalpha1-2GalNAcbeta1-4(NeuAcalpha2-8NeuAcalpha2-3)Galbeta1-4Glcbeta-Cer(d18:1/20:0); Fucalpha1-2GalNAcbeta1-4(NeuAcalpha2-8NeuAcalpha2-3)Galbeta1-4Glcbeta-Cer(d18:1/22:0); Fucalpha1-2GalNAcbeta1-4(NeuAcalpha2-8NeuAcalpha2-3)Galbeta1-4Glcbeta-Cer(d18:1/24:0); Fucalpha1-2GalNAcbeta1-4(NeuAcalpha2-8NeuAcalpha2-3)Galbeta1-4Glcbeta-Cer(d18:1/24:1(15Z)); Fucalpha1-2GalNAcbeta1-4(NeuAcalpha2-8NeuAcalpha2-3)Galbeta1-4Glcbeta-Cer(d18:1/26:0); Fucalpha1-2GalNAcbeta1-4(NeuAcalpha2-8NeuAcalpha2-3)Galbeta1-4Glcbeta-Cer(d18:1/26:1(17Z)); Fucalpha1-3GalNAcbeta1-3Galbeta1-3GalNAcbeta1-4(NeuAcalpha2-3)Galbeta1-4Glcbeta-Cer; Fucalpha1-3GalNAcbeta1-3Galbeta1-3GalNAcbeta1-4(NeuAcalpha2-3)Galbeta1-4Glcbeta-Cer(d18:1/16:0); Fucalpha1-3GalNAcbeta1-3Galbeta1-3GalNAcbeta1-4(NeuAcalpha2-3)Galbeta1-4Glcbeta-Cer(d18:1/18:0); Fucalpha1-3GalNAcbeta1-3Galbeta1-3GalNAcbeta1-4(NeuAcalpha2-3)Galbeta1-4Glcbeta-Cer(d18:1/20:0); Fucalpha1-3GalNAcbeta1-3Galbeta1-3GalNAcbeta1-4(NeuAcalpha2-3)Galbeta1-4Glcbeta-Cer(d18:1/22:0); Fucalpha1-3GalNAcbeta1-3Galbeta1-3GalNAcbeta1-4(NeuAcalpha2-3)Galbeta1-4Glcbeta-Cer(d18:1/24:0); Fucalpha1-3GalNAcbeta1-3Galbeta1-3GalNAcbeta1-4(NeuAcalpha2-3)Galbeta1-4Glcbeta-Cer(d18:1/24:1(15Z)); Fucalpha1-3GalNAcbeta1-3Galbeta1-3GalNAcbeta1-4(NeuAcalpha2-3)Galbeta1-4Glcbeta-Cer(d18:1/26:0); Fucalpha1-3GalNAcbeta1-3Galbeta1-3GalNAcbeta1-4(NeuAcalpha2-3)Galbeta1-4Glcbeta-Cer(d18:1/26:1(17Z)); Fucalpha1-3GlcNAcbeta1-3Galalpha1-3(GalNAcbeta1-4)Galbeta1-4Glcbeta-Cer; Fucalpha1-3GlcNAcbeta1-3Galalpha1-3(GalNAcbeta1-4)Galbeta1-4Glcbeta-Cer(d18:1/16:0); Fucalpha1-3GlcNAcbeta1-3Galalpha1-3(GalNAcbeta1-4)Galbeta1-4Glcbeta-Cer(d18:1/18:0); Fucalpha1-3GlcNAcbeta1-3Galalpha1-3(GalNAcbeta1-4)Galbeta1-4Glcbeta-Cer(d18:1/20:0); Fucalpha1-3GlcNAcbeta1-3Galalpha1-3(GalNAcbeta1-4)Galbeta1-4Glcbeta-Cer(d18:1/22:0); Fucalpha1-3GlcNAcbeta1-3Galalpha1-3(GalNAcbeta1-4)Galbeta1-4Glcbeta-Cer(d18:1/24:0); Fucalpha1-3GlcNAcbeta1-3Galalpha1-3(GalNAcbeta1-4)Galbeta1-4Glcbeta-Cer(d18:1/24:1(15Z)); Fucalpha1-3GlcNAcbeta1-3Galalpha1-3(GalNAcbeta1-4)Galbeta1-4Glcbeta-Cer(d18:1/26:0); Fucalpha1-3GlcNAcbeta1-3Galalpha1-3(GalNAcbeta1-4)Galbeta1-4Glcbeta-Cer(d18:1/26:1(17Z)); Fucalpha1-3GlcNAcbeta1-3Galalpha1-3Galbeta1-4Glcbeta-Cer; Fucalpha1-3GlcNAcbeta1-3Galalpha1-3Galbeta1-4Glcbeta-Cer(d18:1/16:0); Fucalpha1-3GlcNAcbeta1-3Galalpha1-3Galbeta1-4Glcbeta-Cer(d18:1/18:0); Fucalpha1-3GlcNAcbeta1-3Galalpha1-3Galbeta1-4Glcbeta-Cer(d18:1/20:0); Fucalpha1-3GlcNAcbeta1-3Galalpha1-3Galbeta1-4Glcbeta-Cer(d18:1/22:0); Fucalpha1-3GlcNAcbeta1-3Galalpha1-3Galbeta1-4Glcbeta-Cer(d18:1/24:0); Fucalpha1-3GlcNAcbeta1-3Galalpha1-3Galbeta1-4Glcbeta-Cer(d18:1/24:1(15Z)); Fucalpha1-3GlcNAcbeta1-3Galalpha1-3Galbeta1-4Glcbeta-Cer(d18:1/26:0); Fucalpha1-3GlcNAcbeta1-3Galalpha1-3Galbeta1-4Glcbeta-Cer(d18:1/26:1(17Z)); Fucalpha1-3GlcNAcbeta1-3Galbeta1-3(Fucalpha1-4)GlcNAcbeta1-3Galbeta1-4Glcbeta-Cer; Fucalpha1-3GlcNAcbeta1-3Galbeta1-3(Fucalpha1-4)GlcNAcbeta1-3Galbeta1-4Glcbeta-Cer(d18:1/16:0); Fucalpha1-3GlcNAcbeta1-3Galbeta1-3(Fucalpha1-4)GlcNAcbeta1-3Galbeta1-4Glcbeta-Cer(d18:1/18:0); Fucalpha1-3GlcNAcbeta1-3Galbeta1-3(Fucalpha1-4)GlcNAcbeta1-3Galbeta1-4Glcbeta-Cer(d18:1/20:0); Fucalpha1-3GlcNAcbeta1-3Galbeta1-3(Fucalpha1-4)GlcNAcbeta1-3Galbeta1-4Glcbeta-Cer(d18:1/22:0); Fucalpha1-3GlcNAcbeta1-3Galbeta1-3(Fucalpha1-4)GlcNAcbeta1-3Galbeta1-4Glcbeta-Cer(d18:1/24:0); Fucalpha1-3GlcNAcbeta1-3Galbeta1-3(Fucalpha1-4)GlcNAcbeta1-3Galbeta1-4Glcbeta-Cer(d18:1/24:1(15Z)); Fucalpha1-3GlcNAcbeta1-3Galbeta1-3(Fucalpha1-4)GlcNAcbeta1-3Galbeta1-4Glcbeta-Cer(d18:1/26:0); Fucalpha1-3GlcNAcbeta1-3Galbeta1-3(Fucalpha1-4)GlcNAcbeta1-3Galbeta1-4Glcbeta-Cer(d18:1/26:1(17Z)); Fucalpha1-3GlcNAcbeta1-3Galbeta1-3GalNAcbeta1-4(NeuAcalpha2-3)Galbeta1-4Glcbeta-Cer; Fucalpha1-3GlcNAcbeta1-3Galbeta1-3GalNAcbeta1-4(NeuAcalpha2-3)Galbeta1-4Glcbeta-Cer(d18:1/16:0); Fucalpha1-3GlcNAcbeta1-3Galbeta1-3GalNAcbeta1-4(NeuAcalpha2-3)Galbeta1-4Glcbeta-Cer(d18:1/18:0); Fucalpha1-3GlcNAcbeta1-3Galbeta1-3GalNAcbeta1-4(NeuAcalpha2-3)Galbeta1-4Glcbeta-Cer(d18:1/20:0); Fucalpha1-3GlcNAcbeta1-3Galbeta1-3GalNAcbeta1-4(NeuAcalpha2-3)Galbeta1-4Glcbeta-Cer(d18:1/22:0); Fucalpha1-3GlcNAcbeta1-3Galbeta1-3GalNAcbeta1-4(NeuAcalpha2-3)Galbeta1-4Glcbeta-Cer(d18:1/24:0); Fucalpha1-3GlcNAcbeta1-3Galbeta1-3GalNAcbeta1-4(NeuAcalpha2-3)Galbeta1-4Glcbeta-Cer(d18:1/24:1(15Z)); Fucalpha1-3GlcNAcbeta1-3Galbeta1-3GalNAcbeta1-4(NeuAcalpha2-3)Galbeta1-4Glcbeta-Cer(d18:1/26:0); Fucalpha1-3GlcNAcbeta1-3Galbeta1-3GalNAcbeta1-4(NeuAcalpha2-3)Galbeta1-4Glcbeta-Cer(d18:1/26:1(17Z)); Fucalpha1-3GlcNAcbeta1-3Galbeta1-3GalNAcbeta1-4Galbeta1-4Glcbeta-Cer; Fucalpha1-3GlcNAcbeta1-3Galbeta1-3GalNAcbeta1-4Galbeta1-4Glcbeta-Cer(d18:1/16:0); Fucalpha1-3GlcNAcbeta1-3Galbeta1-3GalNAcbeta1-4Galbeta1-4Glcbeta-Cer(d18:1/18:0); Fucalpha1-3GlcNAcbeta1-3Galbeta1-3GalNAcbeta1-4Galbeta1-4Glcbeta-Cer(d18:1/20:0); Fucalpha1-3GlcNAcbeta1-3Galbeta1-3GalNAcbeta1-4Galbeta1-4Glcbeta-Cer(d18:1/22:0); Fucalpha1-3GlcNAcbeta1-3Galbeta1-3GalNAcbeta1-4Galbeta1-4Glcbeta-Cer(d18:1/24:0); Fucalpha1-3GlcNAcbeta1-3Galbeta1-3GalNAcbeta1-4Galbeta1-4Glcbeta-Cer(d18:1/24:1(15Z)); Fucalpha1-3GlcNAcbeta1-3Galbeta1-3GalNAcbeta1-4Galbeta1-4Glcbeta-Cer(d18:1/26:0); Fucalpha1-3GlcNAcbeta1-3Galbeta1-3GalNAcbeta1-4Galbeta1-4Glcbeta-Cer(d18:1/26:1(17Z)); Fucalpha1-4GlcNAcbeta1-3Galbeta1-3GlcNAcbeta1-3Galbeta1-4Glcbeta-Cer; Fucalpha1-4GlcNAcbeta1-3Galbeta1-3GlcNAcbeta1-3Galbeta1-4Glcbeta-Cer(d18:1/16:0); Fucalpha1-4GlcNAcbeta1-3Galbeta1-3GlcNAcbeta1-3Galbeta1-4Glcbeta-Cer(d18:1/18:0); Fucalpha1-4GlcNAcbeta1-3Galbeta1-3GlcNAcbeta1-3Galbeta1-4Glcbeta-Cer(d18:1/20:0); Fucalpha1-4GlcNAcbeta1-3Galbeta1-3GlcNAcbeta1-3Galbeta1-4Glcbeta-Cer(d18:1/22:0); Fucalpha1-4GlcNAcbeta1-3Galbeta1-3GlcNAcbeta1-3Galbeta1-4Glcbeta-Cer(d18:1/24:0); Fucalpha1-4GlcNAcbeta1-3Galbeta1-3GlcNAcbeta1-3Galbeta1-4Glcbeta-Cer(d18:1/24:1(15Z)); Fucalpha1-4GlcNAcbeta1-3Galbeta1-3GlcNAcbeta1-3Galbeta1-4Glcbeta-Cer(d18:1/26:0); Fucalpha1-4GlcNAcbeta1-3Galbeta1-3GlcNAcbeta1-3Galbeta1-4Glcbeta-Cer(d18:1/26:1(17Z)); Fucalpha1-4GlcNAcbeta1-3Galbeta1-4(Fucalpha1-3)GlcNAcbeta1-3Galbeta1-4Glcbeta-Cer; Fucalpha1-4GlcNAcbeta1-3Galbeta1-4(Fucalpha1-3)GlcNAcbeta1-3Galbeta1-4Glcbeta-Cer(d18:1/16:0); Fucalpha1-4GlcNAcbeta1-3Galbeta1-4(Fucalpha1-3)GlcNAcbeta1-3Galbeta1-4Glcbeta-Cer(d18:1/18:0); Fucalpha1-4GlcNAcbeta1-3Galbeta1-4(Fucalpha1-3)GlcNAcbeta1-3Galbeta1-4Glcbeta-Cer(d18:1/20:0); Fucalpha1-4GlcNAcbeta1-3Galbeta1-4(Fucalpha1-3)GlcNAcbeta1-3Galbeta1-4Glcbeta-Cer(d18:1/22:0); Fucalpha1-4GlcNAcbeta1-3Galbeta1-4(Fucalpha1-3)GlcNAcbeta1-3Galbeta1-4Glcbeta-Cer(d18:1/24:0); Fucalpha1-4GlcNAcbeta1-3Galbeta1-4(Fucalpha1-3)GlcNAcbeta1-3Galbeta1-4Glcbeta-Cer(d18:1/24:1(15Z)); Fucalpha1-4GlcNAcbeta1-3Galbeta1-4(Fucalpha1-3)GlcNAcbeta1-3Galbeta1-4Glcbeta-Cer(d18:1/26:0); Fucalpha1-4GlcNAcbeta1-3Galbeta1-4(Fucalpha1-3)GlcNAcbeta1-3Galbeta1-4Glcbeta-Cer(d18:1/26:1(17Z)); Fucalpha2-3(Galbeta1-4)GlcNAcbeta1-6GalNAcbeta1-3Galalpha1-4Galbeta1-4Glcbeta-Cer; Fucalpha2-3(Galbeta1-4)GlcNAcbeta1-6GalNAcbeta1-3Galalpha1-4Galbeta1-4Glcbeta-Cer(d18:1/16:0); Fucalpha2-3(Galbeta1-4)GlcNAcbeta1-6GalNAcbeta1-3Galalpha1-4Galbeta1-4Glcbeta-Cer(d18:1/18:0); Fucalpha2-3(Galbeta1-4)GlcNAcbeta1-6GalNAcbeta1-3Galalpha1-4Galbeta1-4Glcbeta-Cer(d18:1/20:0); Fucalpha2-3(Galbeta1-4)GlcNAcbeta1-6GalNAcbeta1-3Galalpha1-4Galbeta1-4Glcbeta-Cer(d18:1/22:0); Fucalpha2-3(Galbeta1-4)GlcNAcbeta1-6GalNAcbeta1-3Galalpha1-4Galbeta1-4Glcbeta-Cer(d18:1/24:0); Fucalpha2-3(Galbeta1-4)GlcNAcbeta1-6GalNAcbeta1-3Galalpha1-4Galbeta1-4Glcbeta-Cer(d18:1/24:1(15Z)); Fucalpha2-3(Galbeta1-4)GlcNAcbeta1-6GalNAcbeta1-3Galalpha1-4Galbeta1-4Glcbeta-Cer(d18:1/26:0); Fucalpha2-3(Galbeta1-4)GlcNAcbeta1-6GalNAcbeta1-3Galalpha1-4Galbeta1-4Glcbeta-Cer(d18:1/26:1(17Z)); Fucalpha2-3Galbeta1-3GalNAcbeta1-4Galalpha1-4Galbeta1-4Glcbeta-Cer; Fucalpha2-3Galbeta1-3GalNAcbeta1-4Galalpha1-4Galbeta1-4Glcbeta-Cer(d18:1/16:0); Fucalpha2-3Galbeta1-3GalNAcbeta1-4Galalpha1-4Galbeta1-4Glcbeta-Cer(d18:1/18:0); Fucalpha2-3Galbeta1-3GalNAcbeta1-4Galalpha1-4Galbeta1-4Glcbeta-Cer(d18:1/20:0); Fucalpha2-3Galbeta1-3GalNAcbeta1-4Galalpha1-4Galbeta1-4Glcbeta-Cer(d18:1/22:0); Fucalpha2-3Galbeta1-3GalNAcbeta1-4Galalpha1-4Galbeta1-4Glcbeta-Cer(d18:1/24:0); Fucalpha2-3Galbeta1-3GalNAcbeta1-4Galalpha1-4Galbeta1-4Glcbeta-Cer(d18:1/24:1(15Z)); Fucalpha2-3Galbeta1-3GalNAcbeta1-4Galalpha1-4Galbeta1-4Glcbeta-Cer(d18:1/26:0); Fucalpha2-3Galbeta1-3GalNAcbeta1-4Galalpha1-4Galbeta1-4Glcbeta-Cer(d18:1/26:1(17Z)); Fucalpha2-3GlcNAcbeta1-6GalNAcbeta1-3Galalpha1-4Galbeta1-4Glcbeta-Cer; Fucalpha2-3GlcNAcbeta1-6GalNAcbeta1-3Galalpha1-4Galbeta1-4Glcbeta-Cer(d18:1/16:0); Fucalpha2-3GlcNAcbeta1-6GalNAcbeta1-3Galalpha1-4Galbeta1-4Glcbeta-Cer(d18:1/18:0); Fucalpha2-3GlcNAcbeta1-6GalNAcbeta1-3Galalpha1-4Galbeta1-4Glcbeta-Cer(d18:1/20:0); Fucalpha2-3GlcNAcbeta1-6GalNAcbeta1-3Galalpha1-4Galbeta1-4Glcbeta-Cer(d18:1/22:0); Fucalpha2-3GlcNAcbeta1-6GalNAcbeta1-3Galalpha1-4Galbeta1-4Glcbeta-Cer(d18:1/24:0); Fucalpha2-3GlcNAcbeta1-6GalNAcbeta1-3Galalpha1-4Galbeta1-4Glcbeta-Cer(d18:1/24:1(15Z)); Fucalpha2-3GlcNAcbeta1-6GalNAcbeta1-3Galalpha1-4Galbeta1-4Glcbeta-Cer(d18:1/26:0); Fucalpha2-3GlcNAcbeta1-6GalNAcbeta1-3Galalpha1-4Galbeta1-4Glcbeta-Cer(d18:1/26:1(17Z)); Galalpha1-3(Fucalpha1-2)Galbeta1-3(Fucalpha1-4)GlcNAcbeta1-3Galbeta1-4Glcbeta-Cer; Galalpha1-3(Fucalpha1-2)Galbeta1-3(Fucalpha1-4)GlcNAcbeta1-3Galbeta1-4Glcbeta-Cer(d18:1/16:0); Galalpha1-3(Fucalpha1-2)Galbeta1-3(Fucalpha1-4)GlcNAcbeta1-3Galbeta1-4Glcbeta-Cer(d18:1/18:0); Galalpha1-3(Fucalpha1-2)Galbeta1-3(Fucalpha1-4)GlcNAcbeta1-3Galbeta1-4Glcbeta-Cer(d18:1/20:0); Galalpha1-3(Fucalpha1-2)Galbeta1-3(Fucalpha1-4)GlcNAcbeta1-3Galbeta1-4Glcbeta-Cer(d18:1/22:0); Galalpha1-3(Fucalpha1-2)Galbeta1-3(Fucalpha1-4)GlcNAcbeta1-3Galbeta1-4Glcbeta-Cer(d18:1/24:0); Galalpha1-3(Fucalpha1-2)Galbeta1-3(Fucalpha1-4)GlcNAcbeta1-3Galbeta1-4Glcbeta-Cer(d18:1/24:1(15Z)); Galalpha1-3(Fucalpha1-2)Galbeta1-3(Fucalpha1-4)GlcNAcbeta1-3Galbeta1-4Glcbeta-Cer(d18:1/26:0); Galalpha1-3(Fucalpha1-2)Galbeta1-3(Fucalpha1-4)GlcNAcbeta1-3Galbeta1-4Glcbeta-Cer(d18:1/26:1(17Z)); Galalpha1-3(Fucalpha1-2)Galbeta1-3GalNAcbeta1-3Galalpha1-3Galbeta1-4Glcbeta-Cer; Galalpha1-3(Fucalpha1-2)Galbeta1-3GalNAcbeta1-3Galalpha1-3Galbeta1-4Glcbeta-Cer(d18:1/16:0); Galalpha1-3(Fucalpha1-2)Galbeta1-3GalNAcbeta1-3Galalpha1-3Galbeta1-4Glcbeta-Cer(d18:1/18:0); Galalpha1-3(Fucalpha1-2)Galbeta1-3GalNAcbeta1-3Galalpha1-3Galbeta1-4Glcbeta-Cer(d18:1/20:0); Galalpha1-3(Fucalpha1-2)Galbeta1-3GalNAcbeta1-3Galalpha1-3Galbeta1-4Glcbeta-Cer(d18:1/22:0); Galalpha1-3(Fucalpha1-2)Galbeta1-3GalNAcbeta1-3Galalpha1-3Galbeta1-4Glcbeta-Cer(d18:1/24:0); Galalpha1-3(Fucalpha1-2)Galbeta1-3GalNAcbeta1-3Galalpha1-3Galbeta1-4Glcbeta-Cer(d18:1/24:1(15Z)); Galalpha1-3(Fucalpha1-2)Galbeta1-3GalNAcbeta1-3Galalpha1-3Galbeta1-4Glcbeta-Cer(d18:1/26:0); Galalpha1-3(Fucalpha1-2)Galbeta1-3GalNAcbeta1-3Galalpha1-3Galbeta1-4Glcbeta-Cer(d18:1/26:1(17Z)); Galalpha1-3(Fucalpha1-2)Galbeta1-3GalNAcbeta1-3Galalpha1-4Galbeta1-4Glcbeta-Cer; Galalpha1-3(Fucalpha1-2)Galbeta1-3GalNAcbeta1-3Galalpha1-4Galbeta1-4Glcbeta-Cer(d18:1/16:0); Galalpha1-3(Fucalpha1-2)Galbeta1-3GalNAcbeta1-3Galalpha1-4Galbeta1-4Glcbeta-Cer(d18:1/18:0); Galalpha1-3(Fucalpha1-2)Galbeta1-3GalNAcbeta1-3Galalpha1-4Galbeta1-4Glcbeta-Cer(d18:1/20:0); Galalpha1-3(Fucalpha1-2)Galbeta1-3GalNAcbeta1-3Galalpha1-4Galbeta1-4Glcbeta-Cer(d18:1/22:0); Galalpha1-3(Fucalpha1-2)Galbeta1-3GalNAcbeta1-3Galalpha1-4Galbeta1-4Glcbeta-Cer(d18:1/24:0); Galalpha1-3(Fucalpha1-2)Galbeta1-3GalNAcbeta1-3Galalpha1-4Galbeta1-4Glcbeta-Cer(d18:1/24:1(15Z)); Galalpha1-3(Fucalpha1-2)Galbeta1-3GalNAcbeta1-3Galalpha1-4Galbeta1-4Glcbeta-Cer(d18:1/26:0); Galalpha1-3(Fucalpha1-2)Galbeta1-3GalNAcbeta1-3Galalpha1-4Galbeta1-4Glcbeta-Cer(d18:1/26:1(17Z)); Galalpha1-3(Fucalpha1-2)Galbeta1-3GalNAcbeta1-4(NeuAcalpha2-3)Galbeta1-4Glcbeta-Cer; Galalpha1-3(Fucalpha1-2)Galbeta1-3GalNAcbeta1-4(NeuAcalpha2-3)Galbeta1-4Glcbeta-Cer(d18:1/16:0); Galalpha1-3(Fucalpha1-2)Galbeta1-3GalNAcbeta1-4(NeuAcalpha2-3)Galbeta1-4Glcbeta-Cer(d18:1/18:0); Galalpha1-3(Fucalpha1-2)Galbeta1-3GalNAcbeta1-4(NeuAcalpha2-3)Galbeta1-4Glcbeta-Cer(d18:1/20:0); Galalpha1-3(Fucalpha1-2)Galbeta1-3GalNAcbeta1-4(NeuAcalpha2-3)Galbeta1-4Glcbeta-Cer(d18:1/22:0); Galalpha1-3(Fucalpha1-2)Galbeta1-3GalNAcbeta1-4(NeuAcalpha2-3)Galbeta1-4Glcbeta-Cer(d18:1/24:0); Galalpha1-3(Fucalpha1-2)Galbeta1-3GalNAcbeta1-4(NeuAcalpha2-3)Galbeta1-4Glcbeta-Cer(d18:1/24:1(15Z)); Galalpha1-3(Fucalpha1-2)Galbeta1-3GalNAcbeta1-4(NeuAcalpha2-3)Galbeta1-4Glcbeta-Cer(d18:1/26:0); Galalpha1-3(Fucalpha1-2)Galbeta1-3GalNAcbeta1-4(NeuAcalpha2-3)Galbeta1-4Glcbeta-Cer(d18:1/26:1(17Z)); Galalpha1-3(Fucalpha1-2)Galbeta1-3GalNAcbeta1-4(NeuAcalpha2-8NeuAcalpha2-3)Galbeta1-4Glcbeta-Cer; Galalpha1-3(Fucalpha1-2)Galbeta1-3GalNAcbeta1-4(NeuAcalpha2-8NeuAcalpha2-3)Galbeta1-4Glcbeta-Cer(d18:1/16:0); Galalpha1-3(Fucalpha1-2)Galbeta1-3GalNAcbeta1-4(NeuAcalpha2-8NeuAcalpha2-3)Galbeta1-4Glcbeta-Cer(d18:1/18:0); Galalpha1-3(Fucalpha1-2)Galbeta1-3GalNAcbeta1-4(NeuAcalpha2-8NeuAcalpha2-3)Galbeta1-4Glcbeta-Cer(d18:1/20:0); Galalpha1-3(Fucalpha1-2)Galbeta1-3GalNAcbeta1-4(NeuAcalpha2-8NeuAcalpha2-3)Galbeta1-4Glcbeta-Cer(d18:1/22:0); Galalpha1-3(Fucalpha1-2)Galbeta1-3GalNAcbeta1-4(NeuAcalpha2-8NeuAcalpha2-3)Galbeta1-4Glcbeta-Cer(d18:1/24:0); Galalpha1-3(Fucalpha1-2)Galbeta1-3GalNAcbeta1-4(NeuAcalpha2-8NeuAcalpha2-3)Galbeta1-4Glcbeta-Cer(d18:1/24:1(15Z)); Galalpha1-3(Fucalpha1-2)Galbeta1-3GalNAcbeta1-4(NeuAcalpha2-8NeuAcalpha2-3)Galbeta1-4Glcbeta-Cer(d18:1/26:0); Galalpha1-3(Fucalpha1-2)Galbeta1-3GalNAcbeta1-4(NeuAcalpha2-8NeuAcalpha2-3)Galbeta1-4Glcbeta-Cer(d18:1/26:1(17Z)); Galalpha1-3(Fucalpha1-2)Galbeta1-3GalNAcbeta1-4Galbeta1-4Glcbeta-Cer; Galalpha1-3(Fucalpha1-2)Galbeta1-3GalNAcbeta1-4Galbeta1-4Glcbeta-Cer(d18:1/16:0); Galalpha1-3(Fucalpha1-2)Galbeta1-3GalNAcbeta1-4Galbeta1-4Glcbeta-Cer(d18:1/18:0); Galalpha1-3(Fucalpha1-2)Galbeta1-3GalNAcbeta1-4Galbeta1-4Glcbeta-Cer(d18:1/20:0); Galalpha1-3(Fucalpha1-2)Galbeta1-3GalNAcbeta1-4Galbeta1-4Glcbeta-Cer(d18:1/22:0); Galalpha1-3(Fucalpha1-2)Galbeta1-3GalNAcbeta1-4Galbeta1-4Glcbeta-Cer(d18:1/24:0); Galalpha1-3(Fucalpha1-2)Galbeta1-3GalNAcbeta1-4Galbeta1-4Glcbeta-Cer(d18:1/24:1(15Z)); Galalpha1-3(Fucalpha1-2)Galbeta1-3GalNAcbeta1-4Galbeta1-4Glcbeta-Cer(d18:1/26:0); Galalpha1-3(Fucalpha1-2)Galbeta1-3GalNAcbeta1-4Galbeta1-4Glcbeta-Cer(d18:1/26:1(17Z)); Galalpha1-3(Fucalpha1-2)Galbeta1-3GlcNAcbeta1-3Galbeta1-4Glcbeta-Cer; Galalpha1-3(Fucalpha1-2)Galbeta1-3GlcNAcbeta1-3Galbeta1-4Glcbeta-Cer(d18:1/16:0); Galalpha1-3(Fucalpha1-2)Galbeta1-3GlcNAcbeta1-3Galbeta1-4Glcbeta-Cer(d18:1/18:0); Galalpha1-3(Fucalpha1-2)Galbeta1-3GlcNAcbeta1-3Galbeta1-4Glcbeta-Cer(d18:1/20:0); Galalpha1-3(Fucalpha1-2)Galbeta1-3GlcNAcbeta1-3Galbeta1-4Glcbeta-Cer(d18:1/22:0); Galalpha1-3(Fucalpha1-2)Galbeta1-3GlcNAcbeta1-3Galbeta1-4Glcbeta-Cer(d18:1/24:0); Galalpha1-3(Fucalpha1-2)Galbeta1-3GlcNAcbeta1-3Galbeta1-4Glcbeta-Cer(d18:1/24:1(15Z)); Galalpha1-3(Fucalpha1-2)Galbeta1-3GlcNAcbeta1-3Galbeta1-4Glcbeta-Cer(d18:1/26:0); Galalpha1-3(Fucalpha1-2)Galbeta1-3GlcNAcbeta1-3Galbeta1-4Glcbeta-Cer(d18:1/26:1(17Z)); Galalpha1-3(Fucalpha1-2)Galbeta1-4(Fucalpha1-3)GlcNAcbeta1-3Galbeta1-4Glcbeta-Cer; Galalpha1-3(Fucalpha1-2)Galbeta1-4(Fucalpha1-3)GlcNAcbeta1-3Galbeta1-4Glcbeta-Cer(d18:1/16:0); Galalpha1-3(Fucalpha1-2)Galbeta1-4(Fucalpha1-3)GlcNAcbeta1-3Galbeta1-4Glcbeta-Cer(d18:1/18:0); Galalpha1-3(Fucalpha1-2)Galbeta1-4(Fucalpha1-3)GlcNAcbeta1-3Galbeta1-4Glcbeta-Cer(d18:1/20:0); Galalpha1-3(Fucalpha1-2)Galbeta1-4(Fucalpha1-3)GlcNAcbeta1-3Galbeta1-4Glcbeta-Cer(d18:1/22:0); Galalpha1-3(Fucalpha1-2)Galbeta1-4(Fucalpha1-3)GlcNAcbeta1-3Galbeta1-4Glcbeta-Cer(d18:1/24:0); Galalpha1-3(Fucalpha1-2)Galbeta1-4(Fucalpha1-3)GlcNAcbeta1-3Galbeta1-4Glcbeta-Cer(d18:1/24:1(15Z)); Galalpha1-3(Fucalpha1-2)Galbeta1-4(Fucalpha1-3)GlcNAcbeta1-3Galbeta1-4Glcbeta-Cer(d18:1/26:0); Galalpha1-3(Fucalpha1-2)Galbeta1-4(Fucalpha1-3)GlcNAcbeta1-3Galbeta1-4Glcbeta-Cer(d18:1/26:1(17Z)); Galalpha1-3(Fucalpha1-2)Galbeta1-4Glcbeta-Cer; Galalpha1-3(Fucalpha1-2)Galbeta1-4Glcbeta-Cer(d18:1/16:0); Galalpha1-3(Fucalpha1-2)Galbeta1-4Glcbeta-Cer(d18:1/18:0); Galalpha1-3(Fucalpha1-2)Galbeta1-4Glcbeta-Cer(d18:1/20:0); Galalpha1-3(Fucalpha1-2)Galbeta1-4Glcbeta-Cer(d18:1/22:0); Galalpha1-3(Fucalpha1-2)Galbeta1-4Glcbeta-Cer(d18:1/24:0); Galalpha1-3(Fucalpha1-2)Galbeta1-4Glcbeta-Cer(d18:1/24:1(15Z)); Galalpha1-3(Fucalpha1-2)Galbeta1-4Glcbeta-Cer(d18:1/26:0); Galalpha1-3(Fucalpha1-2)Galbeta1-4Glcbeta-Cer(d18:1/26:1(17Z)); Galalpha1-3(Fucalpha1-2)Galbeta1-4GlcNAcbeta1-3(Galalpha1-3(Fucalpha1-2)Galbeta1-4GlcNAcbeta1-6)Galbeta1-4GlcNAcbeta1-3Galbeta1-4Glcbeta-Cer; Galalpha1-3(Fucalpha1-2)Galbeta1-4GlcNAcbeta1-3(Galalpha1-3(Fucalpha1-2)Galbeta1-4GlcNAcbeta1-6)Galbeta1-4GlcNAcbeta1-3Galbeta1-4Glcbeta-Cer(d18:1/16:0); Galalpha1-3(Fucalpha1-2)Galbeta1-4GlcNAcbeta1-3(Galalpha1-3(Fucalpha1-2)Galbeta1-4GlcNAcbeta1-6)Galbeta1-4GlcNAcbeta1-3Galbeta1-4Glcbeta-Cer(d18:1/18:0); Galalpha1-3(Fucalpha1-2)Galbeta1-4GlcNAcbeta1-3(Galalpha1-3(Fucalpha1-2)Galbeta1-4GlcNAcbeta1-6)Galbeta1-4GlcNAcbeta1-3Galbeta1-4Glcbeta-Cer(d18:1/20:0); Galalpha1-3(Fucalpha1-2)Galbeta1-4GlcNAcbeta1-3(Galalpha1-3(Fucalpha1-2)Galbeta1-4GlcNAcbeta1-6)Galbeta1-4GlcNAcbeta1-3Galbeta1-4Glcbeta-Cer(d18:1/22:0); Galalpha1-3(Fucalpha1-2)Galbeta1-4GlcNAcbeta1-3(Galalpha1-3(Fucalpha1-2)Galbeta1-4GlcNAcbeta1-6)Galbeta1-4GlcNAcbeta1-3Galbeta1-4Glcbeta-Cer(d18:1/24:0); Galalpha1-3(Fucalpha1-2)Galbeta1-4GlcNAcbeta1-3(Galalpha1-3(Fucalpha1-2)Galbeta1-4GlcNAcbeta1-6)Galbeta1-4GlcNAcbeta1-3Galbeta1-4Glcbeta-Cer(d18:1/24:1(15Z)); Galalpha1-3(Fucalpha1-2)Galbeta1-4GlcNAcbeta1-3(Galalpha1-3(Fucalpha1-2)Galbeta1-4GlcNAcbeta1-6)Galbeta1-4GlcNAcbeta1-3Galbeta1-4Glcbeta-Cer(d18:1/26:0); Galalpha1-3(Fucalpha1-2)Galbeta1-4GlcNAcbeta1-3(Galalpha1-3(Fucalpha1-2)Galbeta1-4GlcNAcbeta1-6)Galbeta1-4GlcNAcbeta1-3Galbeta1-4Glcbeta-Cer(d18:1/26:1(17Z)); Galalpha1-3(Fucalpha1-2)Galbeta1-4GlcNAcbeta1-3Galbeta1-4Glcbeta-Cer; Galalpha1-3(Fucalpha1-2)Galbeta1-4GlcNAcbeta1-3Galbeta1-4Glcbeta-Cer(d18:1/16:0); Galalpha1-3(Fucalpha1-2)Galbeta1-4GlcNAcbeta1-3Galbeta1-4Glcbeta-Cer(d18:1/18:0); Galalpha1-3(Fucalpha1-2)Galbeta1-4GlcNAcbeta1-3Galbeta1-4Glcbeta-Cer(d18:1/20:0); Galalpha1-3(Fucalpha1-2)Galbeta1-4GlcNAcbeta1-3Galbeta1-4Glcbeta-Cer(d18:1/22:0); Galalpha1-3(Fucalpha1-2)Galbeta1-4GlcNAcbeta1-3Galbeta1-4Glcbeta-Cer(d18:1/24:0); Galalpha1-3(Fucalpha1-2)Galbeta1-4GlcNAcbeta1-3Galbeta1-4Glcbeta-Cer(d18:1/24:1(15Z)); Galalpha1-3(Fucalpha1-2)Galbeta1-4GlcNAcbeta1-3Galbeta1-4Glcbeta-Cer(d18:1/26:0); Galalpha1-3(Fucalpha1-2)Galbeta1-4GlcNAcbeta1-3Galbeta1-4Glcbeta-Cer(d18:1/26:1(17Z)); Galalpha1-3(Fucalpha1-2)Galbeta1-4GlcNAcbeta1-3Galbeta1-4GlcNAcbeta1-3Galbeta1-4Glcbeta-Cer; Galalpha1-3(Fucalpha1-2)Galbeta1-4GlcNAcbeta1-3Galbeta1-4GlcNAcbeta1-3Galbeta1-4Glcbeta-Cer(d18:1/16:0); Galalpha1-3(Fucalpha1-2)Galbeta1-4GlcNAcbeta1-3Galbeta1-4GlcNAcbeta1-3Galbeta1-4Glcbeta-Cer(d18:1/18:0); Galalpha1-3(Fucalpha1-2)Galbeta1-4GlcNAcbeta1-3Galbeta1-4GlcNAcbeta1-3Galbeta1-4Glcbeta-Cer(d18:1/20:0); Galalpha1-3(Fucalpha1-2)Galbeta1-4GlcNAcbeta1-3Galbeta1-4GlcNAcbeta1-3Galbeta1-4Glcbeta-Cer(d18:1/22:0); Galalpha1-3(Fucalpha1-2)Galbeta1-4GlcNAcbeta1-3Galbeta1-4GlcNAcbeta1-3Galbeta1-4Glcbeta-Cer(d18:1/24:0); Galalpha1-3(Fucalpha1-2)Galbeta1-4GlcNAcbeta1-3Galbeta1-4GlcNAcbeta1-3Galbeta1-4Glcbeta-Cer(d18:1/24:1(15Z)); Galalpha1-3(Fucalpha1-2)Galbeta1-4GlcNAcbeta1-3Galbeta1-4GlcNAcbeta1-3Galbeta1-4Glcbeta-Cer(d18:1/26:0); Galalpha1-3(Fucalpha1-2)Galbeta1-4GlcNAcbeta1-3Galbeta1-4GlcNAcbeta1-3Galbeta1-4Glcbeta-Cer(d18:1/26:1(17Z)); Galalpha1-3(Galbeta1-4GlcNAcbeta1-6)Galbeta1-4Glcbeta-Cer; Galalpha1-3(Galbeta1-4GlcNAcbeta1-6)Galbeta1-4Glcbeta-Cer(d18:1/16:0); Galalpha1-3(Galbeta1-4GlcNAcbeta1-6)Galbeta1-4Glcbeta-Cer(d18:1/18:0); Galalpha1-3(Galbeta1-4GlcNAcbeta1-6)Galbeta1-4Glcbeta-Cer(d18:1/20:0); Galalpha1-3(Galbeta1-4GlcNAcbeta1-6)Galbeta1-4Glcbeta-Cer(d18:1/22:0); Galalpha1-3(Galbeta1-4GlcNAcbeta1-6)Galbeta1-4Glcbeta-Cer(d18:1/24:0); Galalpha1-3(Galbeta1-4GlcNAcbeta1-6)Galbeta1-4Glcbeta-Cer(d18:1/24:1(15Z)); Galalpha1-3(Galbeta1-4GlcNAcbeta1-6)Galbeta1-4Glcbeta-Cer(d18:1/26:0); Galalpha1-3(Galbeta1-4GlcNAcbeta1-6)Galbeta1-4Glcbeta-Cer(d18:1/26:1(17Z)); Galalpha1-3(GalNAcbeta1-4)Galbeta1-4Glcbeta-Cer; Galalpha1-3(GalNAcbeta1-4)Galbeta1-4Glcbeta-Cer(d18:1/16:0); Galalpha1-3(GalNAcbeta1-4)Galbeta1-4Glcbeta-Cer(d18:1/18:0); Galalpha1-3(GalNAcbeta1-4)Galbeta1-4Glcbeta-Cer(d18:1/20:0); Galalpha1-3(GalNAcbeta1-4)Galbeta1-4Glcbeta-Cer(d18:1/22:0); Galalpha1-3(GalNAcbeta1-4)Galbeta1-4Glcbeta-Cer(d18:1/24:0); Galalpha1-3(GalNAcbeta1-4)Galbeta1-4Glcbeta-Cer(d18:1/24:1(15Z)); Galalpha1-3(GalNAcbeta1-4)Galbeta1-4Glcbeta-Cer(d18:1/26:0); Galalpha1-3(GalNAcbeta1-4)Galbeta1-4Glcbeta-Cer(d18:1/26:1(17Z)); Galalpha1-3(GalNAcbeta1-4Galbeta1-4GlcNAcbeta1-6)Galbeta1-4Glcbeta-Cer; Galalpha1-3(GalNAcbeta1-4Galbeta1-4GlcNAcbeta1-6)Galbeta1-4Glcbeta-Cer(d18:1/16:0); Galalpha1-3(GalNAcbeta1-4Galbeta1-4GlcNAcbeta1-6)Galbeta1-4Glcbeta-Cer(d18:1/18:0); Galalpha1-3(GalNAcbeta1-4Galbeta1-4GlcNAcbeta1-6)Galbeta1-4Glcbeta-Cer(d18:1/20:0); Galalpha1-3(GalNAcbeta1-4Galbeta1-4GlcNAcbeta1-6)Galbeta1-4Glcbeta-Cer(d18:1/22:0); Galalpha1-3(GalNAcbeta1-4Galbeta1-4GlcNAcbeta1-6)Galbeta1-4Glcbeta-Cer(d18:1/24:0); Galalpha1-3(GalNAcbeta1-4Galbeta1-4GlcNAcbeta1-6)Galbeta1-4Glcbeta-Cer(d18:1/24:1(15Z)); Galalpha1-3(GalNAcbeta1-4Galbeta1-4GlcNAcbeta1-6)Galbeta1-4Glcbeta-Cer(d18:1/26:0); Galalpha1-3(GalNAcbeta1-4Galbeta1-4GlcNAcbeta1-6)Galbeta1-4Glcbeta-Cer(d18:1/26:1(17Z)); Galalpha1-3(GlcNAcbeta1-6)Galbeta1-4Glcbeta-Cer; Galalpha1-3(GlcNAcbeta1-6)Galbeta1-4Glcbeta-Cer(d18:1/16:0); Galalpha1-3(GlcNAcbeta1-6)Galbeta1-4Glcbeta-Cer(d18:1/18:0); Galalpha1-3(GlcNAcbeta1-6)Galbeta1-4Glcbeta-Cer(d18:1/20:0); Galalpha1-3(GlcNAcbeta1-6)Galbeta1-4Glcbeta-Cer(d18:1/22:0); Galalpha1-3(GlcNAcbeta1-6)Galbeta1-4Glcbeta-Cer(d18:1/24:0); Galalpha1-3(GlcNAcbeta1-6)Galbeta1-4Glcbeta-Cer(d18:1/24:1(15Z)); Galalpha1-3(GlcNAcbeta1-6)Galbeta1-4Glcbeta-Cer(d18:1/26:0); Galalpha1-3(GlcNAcbeta1-6)Galbeta1-4Glcbeta-Cer(d18:1/26:1(17Z)); Galalpha1-3(NeuAcalpha2-3(GalNAcbeta1-4)Galbeta1-4GlcNAcbeta1-6)Galbeta1-4Glcbeta-Cer; Galalpha1-3(NeuAcalpha2-3(GalNAcbeta1-4)Galbeta1-4GlcNAcbeta1-6)Galbeta1-4Glcbeta-Cer(d18:1/16:0); Galalpha1-3(NeuAcalpha2-3(GalNAcbeta1-4)Galbeta1-4GlcNAcbeta1-6)Galbeta1-4Glcbeta-Cer(d18:1/18:0); Galalpha1-3(NeuAcalpha2-3(GalNAcbeta1-4)Galbeta1-4GlcNAcbeta1-6)Galbeta1-4Glcbeta-Cer(d18:1/20:0); Galalpha1-3(NeuAcalpha2-3(GalNAcbeta1-4)Galbeta1-4GlcNAcbeta1-6)Galbeta1-4Glcbeta-Cer(d18:1/22:0); Galalpha1-3(NeuAcalpha2-3(GalNAcbeta1-4)Galbeta1-4GlcNAcbeta1-6)Galbeta1-4Glcbeta-Cer(d18:1/24:0); Galalpha1-3(NeuAcalpha2-3(GalNAcbeta1-4)Galbeta1-4GlcNAcbeta1-6)Galbeta1-4Glcbeta-Cer(d18:1/24:1(15Z)); Galalpha1-3(NeuAcalpha2-3(GalNAcbeta1-4)Galbeta1-4GlcNAcbeta1-6)Galbeta1-4Glcbeta-Cer(d18:1/26:0); Galalpha1-3(NeuAcalpha2-3(GalNAcbeta1-4)Galbeta1-4GlcNAcbeta1-6)Galbeta1-4Glcbeta-Cer(d18:1/26:1(17Z)); Galalpha1-3(NeuAcalpha2-3Galbeta1-4GlcNAcbeta1-6)Galbeta1-4Glcbeta-Cer; Galalpha1-3(NeuAcalpha2-3Galbeta1-4GlcNAcbeta1-6)Galbeta1-4Glcbeta-Cer(d18:1/16:0); Galalpha1-3(NeuAcalpha2-3Galbeta1-4GlcNAcbeta1-6)Galbeta1-4Glcbeta-Cer(d18:1/18:0); Galalpha1-3(NeuAcalpha2-3Galbeta1-4GlcNAcbeta1-6)Galbeta1-4Glcbeta-Cer(d18:1/20:0); Galalpha1-3(NeuAcalpha2-3Galbeta1-4GlcNAcbeta1-6)Galbeta1-4Glcbeta-Cer(d18:1/22:0); Galalpha1-3(NeuAcalpha2-3Galbeta1-4GlcNAcbeta1-6)Galbeta1-4Glcbeta-Cer(d18:1/24:0); Galalpha1-3(NeuAcalpha2-3Galbeta1-4GlcNAcbeta1-6)Galbeta1-4Glcbeta-Cer(d18:1/24:1(15Z)); Galalpha1-3(NeuAcalpha2-3Galbeta1-4GlcNAcbeta1-6)Galbeta1-4Glcbeta-Cer(d18:1/26:0); Galalpha1-3(NeuAcalpha2-3Galbeta1-4GlcNAcbeta1-6)Galbeta1-4Glcbeta-Cer(d18:1/26:1(17Z)); Galalpha1-3Galalpha1-3Galalpha1-3Galalpha1-3Galalpha1-3Galalpha1-4Galbeta1-4Glcbeta-Cer; Galalpha1-3Galalpha1-3Galalpha1-3Galalpha1-3Galalpha1-3Galalpha1-4Galbeta1-4Glcbeta-Cer(d18:1/16:0); Galalpha1-3Galalpha1-3Galalpha1-3Galalpha1-3Galalpha1-3Galalpha1-4Galbeta1-4Glcbeta-Cer(d18:1/18:0); Galalpha1-3Galalpha1-3Galalpha1-3Galalpha1-3Galalpha1-3Galalpha1-4Galbeta1-4Glcbeta-Cer(d18:1/20:0); Galalpha1-3Galalpha1-3Galalpha1-3Galalpha1-3Galalpha1-3Galalpha1-4Galbeta1-4Glcbeta-Cer(d18:1/22:0); Galalpha1-3Galalpha1-3Galalpha1-3Galalpha1-3Galalpha1-3Galalpha1-4Galbeta1-4Glcbeta-Cer(d18:1/24:0); Galalpha1-3Galalpha1-3Galalpha1-3Galalpha1-3Galalpha1-3Galalpha1-4Galbeta1-4Glcbeta-Cer(d18:1/24:1(15Z)); Galalpha1-3Galalpha1-3Galalpha1-3Galalpha1-3Galalpha1-3Galalpha1-4Galbeta1-4Glcbeta-Cer(d18:1/26:0); Galalpha1-3Galalpha1-3Galalpha1-3Galalpha1-3Galalpha1-3Galalpha1-4Galbeta1-4Glcbeta-Cer(d18:1/26:1(17Z)); Galalpha1-3Galalpha1-3Galalpha1-3Galalpha1-3Galalpha1-4Galbeta1-4Glcbeta-Cer; Galalpha1-3Galalpha1-3Galalpha1-3Galalpha1-3Galalpha1-4Galbeta1-4Glcbeta-Cer(d18:1/16:0); Galalpha1-3Galalpha1-3Galalpha1-3Galalpha1-3Galalpha1-4Galbeta1-4Glcbeta-Cer(d18:1/18:0); Galalpha1-3Galalpha1-3Galalpha1-3Galalpha1-3Galalpha1-4Galbeta1-4Glcbeta-Cer(d18:1/20:0); Galalpha1-3Galalpha1-3Galalpha1-3Galalpha1-3Galalpha1-4Galbeta1-4Glcbeta-Cer(d18:1/22:0); Galalpha1-3Galalpha1-3Galalpha1-3Galalpha1-3Galalpha1-4Galbeta1-4Glcbeta-Cer(d18:1/24:0); Galalpha1-3Galalpha1-3Galalpha1-3Galalpha1-3Galalpha1-4Galbeta1-4Glcbeta-Cer(d18:1/24:1(15Z)); Galalpha1-3Galalpha1-3Galalpha1-3Galalpha1-3Galalpha1-4Galbeta1-4Glcbeta-Cer(d18:1/26:0); Galalpha1-3Galalpha1-3Galalpha1-3Galalpha1-3Galalpha1-4Galbeta1-4Glcbeta-Cer(d18:1/26:1(17Z)); Galalpha1-3Galalpha1-3Galalpha1-3Galalpha1-4Galbeta1-4Glcbeta-Cer; Galalpha1-3Galalpha1-3Galalpha1-3Galalpha1-4Galbeta1-4Glcbeta-Cer(d18:1/16:0); Galalpha1-3Galalpha1-3Galalpha1-3Galalpha1-4Galbeta1-4Glcbeta-Cer(d18:1/18:0); Galalpha1-3Galalpha1-3Galalpha1-3Galalpha1-4Galbeta1-4Glcbeta-Cer(d18:1/20:0); Galalpha1-3Galalpha1-3Galalpha1-3Galalpha1-4Galbeta1-4Glcbeta-Cer(d18:1/22:0); Galalpha1-3Galalpha1-3Galalpha1-3Galalpha1-4Galbeta1-4Glcbeta-Cer(d18:1/24:0); Galalpha1-3Galalpha1-3Galalpha1-3Galalpha1-4Galbeta1-4Glcbeta-Cer(d18:1/24:1(15Z)); Galalpha1-3Galalpha1-3Galalpha1-3Galalpha1-4Galbeta1-4Glcbeta-Cer(d18:1/26:0); Galalpha1-3Galalpha1-3Galalpha1-3Galalpha1-4Galbeta1-4Glcbeta-Cer(d18:1/26:1(17Z)); Galalpha1-3Galalpha1-3Galalpha1-4Galbeta1-4Glcbeta-Cer; Galalpha1-3Galalpha1-3Galalpha1-4Galbeta1-4Glcbeta-Cer(d18:1/16:0); Galalpha1-3Galalpha1-3Galalpha1-4Galbeta1-4Glcbeta-Cer(d18:1/18:0); Galalpha1-3Galalpha1-3Galalpha1-4Galbeta1-4Glcbeta-Cer(d18:1/20:0); Galalpha1-3Galalpha1-3Galalpha1-4Galbeta1-4Glcbeta-Cer(d18:1/22:0); Galalpha1-3Galalpha1-3Galalpha1-4Galbeta1-4Glcbeta-Cer(d18:1/24:0); Galalpha1-3Galalpha1-3Galalpha1-4Galbeta1-4Glcbeta-Cer(d18:1/24:1(15Z)); Galalpha1-3Galalpha1-3Galalpha1-4Galbeta1-4Glcbeta-Cer(d18:1/26:0); Galalpha1-3Galalpha1-3Galalpha1-4Galbeta1-4Glcbeta-Cer(d18:1/26:1(17Z)); Galalpha1-3Galalpha1-3Galbeta1-3GalNAcbeta1-4(NeuAcalpha2-8NeuAcalpha2-3)Galbeta1-4Glcbeta-Cer; Galalpha1-3Galalpha1-3Galbeta1-3GalNAcbeta1-4(NeuAcalpha2-8NeuAcalpha2-3)Galbeta1-4Glcbeta-Cer(d18:1/16:0); Galalpha1-3Galalpha1-3Galbeta1-3GalNAcbeta1-4(NeuAcalpha2-8NeuAcalpha2-3)Galbeta1-4Glcbeta-Cer(d18:1/18:0); Galalpha1-3Galalpha1-3Galbeta1-3GalNAcbeta1-4(NeuAcalpha2-8NeuAcalpha2-3)Galbeta1-4Glcbeta-Cer(d18:1/20:0); Galalpha1-3Galalpha1-3Galbeta1-3GalNAcbeta1-4(NeuAcalpha2-8NeuAcalpha2-3)Galbeta1-4Glcbeta-Cer(d18:1/22:0); Galalpha1-3Galalpha1-3Galbeta1-3GalNAcbeta1-4(NeuAcalpha2-8NeuAcalpha2-3)Galbeta1-4Glcbeta-Cer(d18:1/24:0); Galalpha1-3Galalpha1-3Galbeta1-3GalNAcbeta1-4(NeuAcalpha2-8NeuAcalpha2-3)Galbeta1-4Glcbeta-Cer(d18:1/24:1(15Z)); Galalpha1-3Galalpha1-3Galbeta1-3GalNAcbeta1-4(NeuAcalpha2-8NeuAcalpha2-3)Galbeta1-4Glcbeta-Cer(d18:1/26:0); Galalpha1-3Galalpha1-3Galbeta1-3GalNAcbeta1-4(NeuAcalpha2-8NeuAcalpha2-3)Galbeta1-4Glcbeta-Cer(d18:1/26:1(17Z)); Galalpha1-3Galalpha1-3Galbeta1-4Glcbeta-Cer; Galalpha1-3Galalpha1-3Galbeta1-4Glcbeta-Cer(d18:1/16:0); Galalpha1-3Galalpha1-3Galbeta1-4Glcbeta-Cer(d18:1/18:0); Galalpha1-3Galalpha1-3Galbeta1-4Glcbeta-Cer(d18:1/20:0); Galalpha1-3Galalpha1-3Galbeta1-4Glcbeta-Cer(d18:1/22:0); Galalpha1-3Galalpha1-3Galbeta1-4Glcbeta-Cer(d18:1/24:0); Galalpha1-3Galalpha1-3Galbeta1-4Glcbeta-Cer(d18:1/24:1(15Z)); Galalpha1-3Galalpha1-3Galbeta1-4Glcbeta-Cer(d18:1/26:0); Galalpha1-3Galalpha1-3Galbeta1-4Glcbeta-Cer(d18:1/26:1(17Z)); Galalpha1-3Galalpha1-4Galbeta1-4Glcbeta-Cer; Galalpha1-3Galalpha1-4Galbeta1-4Glcbeta-Cer(d18:1/16:0); Galalpha1-3Galalpha1-4Galbeta1-4Glcbeta-Cer(d18:1/18:0); Galalpha1-3Galalpha1-4Galbeta1-4Glcbeta-Cer(d18:1/20:0); Galalpha1-3Galalpha1-4Galbeta1-4Glcbeta-Cer(d18:1/22:0); Galalpha1-3Galalpha1-4Galbeta1-4Glcbeta-Cer(d18:1/24:0); Galalpha1-3Galalpha1-4Galbeta1-4Glcbeta-Cer(d18:1/24:1(15Z)); Galalpha1-3Galalpha1-4Galbeta1-4Glcbeta-Cer(d18:1/26:0); Galalpha1-3Galalpha1-4Galbeta1-4Glcbeta-Cer(d18:1/26:1(17Z)); Galalpha1-3Galbeta1-3GalNAcbeta1-3Galalpha1-3Galbeta1-4Glcbeta-Cer; Galalpha1-3Galbeta1-3GalNAcbeta1-3Galalpha1-3Galbeta1-4Glcbeta-Cer(d18:1/16:0); Galalpha1-3Galbeta1-3GalNAcbeta1-3Galalpha1-3Galbeta1-4Glcbeta-Cer(d18:1/18:0); Galalpha1-3Galbeta1-3GalNAcbeta1-3Galalpha1-3Galbeta1-4Glcbeta-Cer(d18:1/20:0); Galalpha1-3Galbeta1-3GalNAcbeta1-3Galalpha1-3Galbeta1-4Glcbeta-Cer(d18:1/22:0); Galalpha1-3Galbeta1-3GalNAcbeta1-3Galalpha1-3Galbeta1-4Glcbeta-Cer(d18:1/24:0); Galalpha1-3Galbeta1-3GalNAcbeta1-3Galalpha1-3Galbeta1-4Glcbeta-Cer(d18:1/24:1(15Z)); Galalpha1-3Galbeta1-3GalNAcbeta1-3Galalpha1-3Galbeta1-4Glcbeta-Cer(d18:1/26:0); Galalpha1-3Galbeta1-3GalNAcbeta1-3Galalpha1-3Galbeta1-4Glcbeta-Cer(d18:1/26:1(17Z)); Galalpha1-3Galbeta1-3GalNAcbeta1-3Galalpha1-4Galbeta1-4Glcbeta-Cer; Galalpha1-3Galbeta1-3GalNAcbeta1-3Galalpha1-4Galbeta1-4Glcbeta-Cer(d18:1/16:0); Galalpha1-3Galbeta1-3GalNAcbeta1-3Galalpha1-4Galbeta1-4Glcbeta-Cer(d18:1/18:0); Galalpha1-3Galbeta1-3GalNAcbeta1-3Galalpha1-4Galbeta1-4Glcbeta-Cer(d18:1/20:0); Galalpha1-3Galbeta1-3GalNAcbeta1-3Galalpha1-4Galbeta1-4Glcbeta-Cer(d18:1/22:0); Galalpha1-3Galbeta1-3GalNAcbeta1-3Galalpha1-4Galbeta1-4Glcbeta-Cer(d18:1/24:0); Galalpha1-3Galbeta1-3GalNAcbeta1-3Galalpha1-4Galbeta1-4Glcbeta-Cer(d18:1/24:1(15Z)); Galalpha1-3Galbeta1-3GalNAcbeta1-3Galalpha1-4Galbeta1-4Glcbeta-Cer(d18:1/26:0); Galalpha1-3Galbeta1-3GalNAcbeta1-3Galalpha1-4Galbeta1-4Glcbeta-Cer(d18:1/26:1(17Z)); Galalpha1-3Galbeta1-3GalNAcbeta1-4(NeuAcalpha2-8NeuAcalpha2-3)Galbeta1-4Glcbeta-Cer; Galalpha1-3Galbeta1-3GalNAcbeta1-4(NeuAcalpha2-8NeuAcalpha2-3)Galbeta1-4Glcbeta-Cer(d18:1/16:0); Galalpha1-3Galbeta1-3GalNAcbeta1-4(NeuAcalpha2-8NeuAcalpha2-3)Galbeta1-4Glcbeta-Cer(d18:1/18:0); Galalpha1-3Galbeta1-3GalNAcbeta1-4(NeuAcalpha2-8NeuAcalpha2-3)Galbeta1-4Glcbeta-Cer(d18:1/20:0); Galalpha1-3Galbeta1-3GalNAcbeta1-4(NeuAcalpha2-8NeuAcalpha2-3)Galbeta1-4Glcbeta-Cer(d18:1/22:0); Galalpha1-3Galbeta1-3GalNAcbeta1-4(NeuAcalpha2-8NeuAcalpha2-3)Galbeta1-4Glcbeta-Cer(d18:1/24:0); Galalpha1-3Galbeta1-3GalNAcbeta1-4(NeuAcalpha2-8NeuAcalpha2-3)Galbeta1-4Glcbeta-Cer(d18:1/24:1(15Z)); Galalpha1-3Galbeta1-3GalNAcbeta1-4(NeuAcalpha2-8NeuAcalpha2-3)Galbeta1-4Glcbeta-Cer(d18:1/26:0); Galalpha1-3Galbeta1-3GalNAcbeta1-4(NeuAcalpha2-8NeuAcalpha2-3)Galbeta1-4Glcbeta-Cer(d18:1/26:1(17Z)); Galalpha1-3Galbeta1-3GalNAcbeta1-4Galbeta1-4Glcbeta-Cer; Galalpha1-3Galbeta1-3GalNAcbeta1-4Galbeta1-4Glcbeta-Cer(d18:1/16:0); Galalpha1-3Galbeta1-3GalNAcbeta1-4Galbeta1-4Glcbeta-Cer(d18:1/18:0); Galalpha1-3Galbeta1-3GalNAcbeta1-4Galbeta1-4Glcbeta-Cer(d18:1/20:0); Galalpha1-3Galbeta1-3GalNAcbeta1-4Galbeta1-4Glcbeta-Cer(d18:1/22:0); Galalpha1-3Galbeta1-3GalNAcbeta1-4Galbeta1-4Glcbeta-Cer(d18:1/24:0); Galalpha1-3Galbeta1-3GalNAcbeta1-4Galbeta1-4Glcbeta-Cer(d18:1/24:1(15Z)); Galalpha1-3Galbeta1-3GalNAcbeta1-4Galbeta1-4Glcbeta-Cer(d18:1/26:0); Galalpha1-3Galbeta1-3GalNAcbeta1-4Galbeta1-4Glcbeta-Cer(d18:1/26:1(17Z)); Galalpha1-3Galbeta1-3GlcNAcbeta1-3Galbeta1-4Glcbeta-Cer; Galalpha1-3Galbeta1-3GlcNAcbeta1-3Galbeta1-4Glcbeta-Cer(d18:1/16:0); Galalpha1-3Galbeta1-3GlcNAcbeta1-3Galbeta1-4Glcbeta-Cer(d18:1/18:0); Galalpha1-3Galbeta1-3GlcNAcbeta1-3Galbeta1-4Glcbeta-Cer(d18:1/20:0); Galalpha1-3Galbeta1-3GlcNAcbeta1-3Galbeta1-4Glcbeta-Cer(d18:1/22:0); Galalpha1-3Galbeta1-3GlcNAcbeta1-3Galbeta1-4Glcbeta-Cer(d18:1/24:0); Galalpha1-3Galbeta1-3GlcNAcbeta1-3Galbeta1-4Glcbeta-Cer(d18:1/24:1(15Z)); Galalpha1-3Galbeta1-3GlcNAcbeta1-3Galbeta1-4Glcbeta-Cer(d18:1/26:0); Galalpha1-3Galbeta1-3GlcNAcbeta1-3Galbeta1-4Glcbeta-Cer(d18:1/26:1(17Z)); Galalpha1-3Galbeta1-4(Fucalpha1-3)GlcNAcbeta1-3Galbeta1-4Glcbeta-Cer; Galalpha1-3Galbeta1-4(Fucalpha1-3)GlcNAcbeta1-3Galbeta1-4Glcbeta-Cer(d18:1/16:0); Galalpha1-3Galbeta1-4(Fucalpha1-3)GlcNAcbeta1-3Galbeta1-4Glcbeta-Cer(d18:1/18:0); Galalpha1-3Galbeta1-4(Fucalpha1-3)GlcNAcbeta1-3Galbeta1-4Glcbeta-Cer(d18:1/20:0); Galalpha1-3Galbeta1-4(Fucalpha1-3)GlcNAcbeta1-3Galbeta1-4Glcbeta-Cer(d18:1/22:0); Galalpha1-3Galbeta1-4(Fucalpha1-3)GlcNAcbeta1-3Galbeta1-4Glcbeta-Cer(d18:1/24:0); Galalpha1-3Galbeta1-4(Fucalpha1-3)GlcNAcbeta1-3Galbeta1-4Glcbeta-Cer(d18:1/24:1(15Z)); Galalpha1-3Galbeta1-4(Fucalpha1-3)GlcNAcbeta1-3Galbeta1-4Glcbeta-Cer(d18:1/26:0); Galalpha1-3Galbeta1-4(Fucalpha1-3)GlcNAcbeta1-3Galbeta1-4Glcbeta-Cer(d18:1/26:1(17Z)); Galalpha1-3Galbeta1-4Glcbeta-Cer; Galalpha1-3Galbeta1-4Glcbeta-Cer(d18:1/16:0); Galalpha1-3Galbeta1-4Glcbeta-Cer(d18:1/18:0); Galalpha1-3Galbeta1-4Glcbeta-Cer(d18:1/20:0); Galalpha1-3Galbeta1-4Glcbeta-Cer(d18:1/22:0); Galalpha1-3Galbeta1-4Glcbeta-Cer(d18:1/24:0); Galalpha1-3Galbeta1-4Glcbeta-Cer(d18:1/24:1(15Z)); Galalpha1-3Galbeta1-4Glcbeta-Cer(d18:1/26:0); Galalpha1-3Galbeta1-4Glcbeta-Cer(d18:1/26:1(17Z)); Galalpha1-3Galbeta1-4GlcNAcbeta1-3(Fucalpha1-2(Galalpha1-3)Galbeta1-4GlcNAcbeta1-6)Galbeta1-4GlcNAcbeta1-3Galbeta1-4Glcbeta-Cer; Galalpha1-3Galbeta1-4GlcNAcbeta1-3(Fucalpha1-2(Galalpha1-3)Galbeta1-4GlcNAcbeta1-6)Galbeta1-4GlcNAcbeta1-3Galbeta1-4Glcbeta-Cer(d18:1/16:0); Galalpha1-3Galbeta1-4GlcNAcbeta1-3(Fucalpha1-2(Galalpha1-3)Galbeta1-4GlcNAcbeta1-6)Galbeta1-4GlcNAcbeta1-3Galbeta1-4Glcbeta-Cer(d18:1/18:0); Galalpha1-3Galbeta1-4GlcNAcbeta1-3(Fucalpha1-2(Galalpha1-3)Galbeta1-4GlcNAcbeta1-6)Galbeta1-4GlcNAcbeta1-3Galbeta1-4Glcbeta-Cer(d18:1/20:0); Galalpha1-3Galbeta1-4GlcNAcbeta1-3(Fucalpha1-2(Galalpha1-3)Galbeta1-4GlcNAcbeta1-6)Galbeta1-4GlcNAcbeta1-3Galbeta1-4Glcbeta-Cer(d18:1/22:0); Galalpha1-3Galbeta1-4GlcNAcbeta1-3(Fucalpha1-2(Galalpha1-3)Galbeta1-4GlcNAcbeta1-6)Galbeta1-4GlcNAcbeta1-3Galbeta1-4Glcbeta-Cer(d18:1/24:0); Galalpha1-3Galbeta1-4GlcNAcbeta1-3(Fucalpha1-2(Galalpha1-3)Galbeta1-4GlcNAcbeta1-6)Galbeta1-4GlcNAcbeta1-3Galbeta1-4Glcbeta-Cer(d18:1/24:1(15Z)); Galalpha1-3Galbeta1-4GlcNAcbeta1-3(Fucalpha1-2(Galalpha1-3)Galbeta1-4GlcNAcbeta1-6)Galbeta1-4GlcNAcbeta1-3Galbeta1-4Glcbeta-Cer(d18:1/26:0); Galalpha1-3Galbeta1-4GlcNAcbeta1-3(Fucalpha1-2(Galalpha1-3)Galbeta1-4GlcNAcbeta1-6)Galbeta1-4GlcNAcbeta1-3Galbeta1-4Glcbeta-Cer(d18:1/26:1(17Z)); Galalpha1-3Galbeta1-4GlcNAcbeta1-3(Galalpha1-3Galbeta1-4GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer; Galalpha1-3Galbeta1-4GlcNAcbeta1-3(Galalpha1-3Galbeta1-4GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/16:0); Galalpha1-3Galbeta1-4GlcNAcbeta1-3(Galalpha1-3Galbeta1-4GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/18:0); Galalpha1-3Galbeta1-4GlcNAcbeta1-3(Galalpha1-3Galbeta1-4GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/20:0); Galalpha1-3Galbeta1-4GlcNAcbeta1-3(Galalpha1-3Galbeta1-4GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/22:0); Galalpha1-3Galbeta1-4GlcNAcbeta1-3(Galalpha1-3Galbeta1-4GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:0); Galalpha1-3Galbeta1-4GlcNAcbeta1-3(Galalpha1-3Galbeta1-4GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:1(15Z)); Galalpha1-3Galbeta1-4GlcNAcbeta1-3(Galalpha1-3Galbeta1-4GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:0); Galalpha1-3Galbeta1-4GlcNAcbeta1-3(Galalpha1-3Galbeta1-4GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:1(17Z)); Galalpha1-3Galbeta1-4GlcNAcbeta1-3(Galalpha1-3Galbeta1-4GlcNAcbeta1-6)Galbeta1-4GlcNAcbeta1-3(Galbeta1-4GlcNAcbeta1-6)Galbeta1-4GlcNAcbeta1-3Galbeta1-4Glcbeta-Cer; Galalpha1-3Galbeta1-4GlcNAcbeta1-3(Galalpha1-3Galbeta1-4GlcNAcbeta1-6)Galbeta1-4GlcNAcbeta1-3(Galbeta1-4GlcNAcbeta1-6)Galbeta1-4GlcNAcbeta1-3Galbeta1-4Glcbeta-Cer(d18:1/16:0); Galalpha1-3Galbeta1-4GlcNAcbeta1-3(Galalpha1-3Galbeta1-4GlcNAcbeta1-6)Galbeta1-4GlcNAcbeta1-3(Galbeta1-4GlcNAcbeta1-6)Galbeta1-4GlcNAcbeta1-3Galbeta1-4Glcbeta-Cer(d18:1/18:0); Galalpha1-3Galbeta1-4GlcNAcbeta1-3(Galalpha1-3Galbeta1-4GlcNAcbeta1-6)Galbeta1-4GlcNAcbeta1-3(Galbeta1-4GlcNAcbeta1-6)Galbeta1-4GlcNAcbeta1-3Galbeta1-4Glcbeta-Cer(d18:1/20:0); Galalpha1-3Galbeta1-4GlcNAcbeta1-3(Galalpha1-3Galbeta1-4GlcNAcbeta1-6)Galbeta1-4GlcNAcbeta1-3(Galbeta1-4GlcNAcbeta1-6)Galbeta1-4GlcNAcbeta1-3Galbeta1-4Glcbeta-Cer(d18:1/22:0); Galalpha1-3Galbeta1-4GlcNAcbeta1-3(Galalpha1-3Galbeta1-4GlcNAcbeta1-6)Galbeta1-4GlcNAcbeta1-3(Galbeta1-4GlcNAcbeta1-6)Galbeta1-4GlcNAcbeta1-3Galbeta1-4Glcbeta-Cer(d18:1/24:0); Galalpha1-3Galbeta1-4GlcNAcbeta1-3(Galalpha1-3Galbeta1-4GlcNAcbeta1-6)Galbeta1-4GlcNAcbeta1-3(Galbeta1-4GlcNAcbeta1-6)Galbeta1-4GlcNAcbeta1-3Galbeta1-4Glcbeta-Cer(d18:1/24:1(15Z)); Galalpha1-3Galbeta1-4GlcNAcbeta1-3(Galalpha1-3Galbeta1-4GlcNAcbeta1-6)Galbeta1-4GlcNAcbeta1-3(Galbeta1-4GlcNAcbeta1-6)Galbeta1-4GlcNAcbeta1-3Galbeta1-4Glcbeta-Cer(d18:1/26:0); Galalpha1-3Galbeta1-4GlcNAcbeta1-3(Galalpha1-3Galbeta1-4GlcNAcbeta1-6)Galbeta1-4GlcNAcbeta1-3(Galbeta1-4GlcNAcbeta1-6)Galbeta1-4GlcNAcbeta1-3Galbeta1-4Glcbeta-Cer(d18:1/26:1(17Z)); Galalpha1-3Galbeta1-4GlcNAcbeta1-3(Galalpha1-3Galbeta1-4GlcNAcbeta1-6)Galbeta1-4GlcNAcbeta1-3(GlcNAcbeta1-6)Galbeta1-4GlcNAcbeta1-3Galbeta1-4Glcbeta-Cer; Galalpha1-3Galbeta1-4GlcNAcbeta1-3(Galalpha1-3Galbeta1-4GlcNAcbeta1-6)Galbeta1-4GlcNAcbeta1-3(GlcNAcbeta1-6)Galbeta1-4GlcNAcbeta1-3Galbeta1-4Glcbeta-Cer(d18:1/16:0); Galalpha1-3Galbeta1-4GlcNAcbeta1-3(Galalpha1-3Galbeta1-4GlcNAcbeta1-6)Galbeta1-4GlcNAcbeta1-3(GlcNAcbeta1-6)Galbeta1-4GlcNAcbeta1-3Galbeta1-4Glcbeta-Cer(d18:1/18:0); Galalpha1-3Galbeta1-4GlcNAcbeta1-3(Galalpha1-3Galbeta1-4GlcNAcbeta1-6)Galbeta1-4GlcNAcbeta1-3(GlcNAcbeta1-6)Galbeta1-4GlcNAcbeta1-3Galbeta1-4Glcbeta-Cer(d18:1/20:0); Galalpha1-3Galbeta1-4GlcNAcbeta1-3(Galalpha1-3Galbeta1-4GlcNAcbeta1-6)Galbeta1-4GlcNAcbeta1-3(GlcNAcbeta1-6)Galbeta1-4GlcNAcbeta1-3Galbeta1-4Glcbeta-Cer(d18:1/22:0); Galalpha1-3Galbeta1-4GlcNAcbeta1-3(Galalpha1-3Galbeta1-4GlcNAcbeta1-6)Galbeta1-4GlcNAcbeta1-3(GlcNAcbeta1-6)Galbeta1-4GlcNAcbeta1-3Galbeta1-4Glcbeta-Cer(d18:1/24:0); Galalpha1-3Galbeta1-4GlcNAcbeta1-3(Galalpha1-3Galbeta1-4GlcNAcbeta1-6)Galbeta1-4GlcNAcbeta1-3(GlcNAcbeta1-6)Galbeta1-4GlcNAcbeta1-3Galbeta1-4Glcbeta-Cer(d18:1/24:1(15Z)); Galalpha1-3Galbeta1-4GlcNAcbeta1-3(Galalpha1-3Galbeta1-4GlcNAcbeta1-6)Galbeta1-4GlcNAcbeta1-3(GlcNAcbeta1-6)Galbeta1-4GlcNAcbeta1-3Galbeta1-4Glcbeta-Cer(d18:1/26:0); Galalpha1-3Galbeta1-4GlcNAcbeta1-3(Galalpha1-3Galbeta1-4GlcNAcbeta1-6)Galbeta1-4GlcNAcbeta1-3(GlcNAcbeta1-6)Galbeta1-4GlcNAcbeta1-3Galbeta1-4Glcbeta-Cer(d18:1/26:1(17Z)); Galalpha1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer; Galalpha1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/16:0); Galalpha1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/18:0); Galalpha1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/20:0); Galalpha1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/22:0); Galalpha1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:0); Galalpha1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:1(15Z)); Galalpha1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:0); Galalpha1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:1(17Z)); Galalpha1-3Galbeta1-4GlcNAcbeta1-3(Galalpha1-3Galbeta1-4GlcNAcbeta1-6)Galbeta1-4GlcNAcbeta1-3Galbeta1-4GlcNAcbeta1-3Galbeta1-4Glcbeta-Cer; Galalpha1-3Galbeta1-4GlcNAcbeta1-3(Galalpha1-3Galbeta1-4GlcNAcbeta1-6)Galbeta1-4GlcNAcbeta1-3Galbeta1-4GlcNAcbeta1-3Galbeta1-4Glcbeta-Cer(d18:1/16:0); Galalpha1-3Galbeta1-4GlcNAcbeta1-3(Galalpha1-3Galbeta1-4GlcNAcbeta1-6)Galbeta1-4GlcNAcbeta1-3Galbeta1-4GlcNAcbeta1-3Galbeta1-4Glcbeta-Cer(d18:1/18:0); Galalpha1-3Galbeta1-4GlcNAcbeta1-3(Galalpha1-3Galbeta1-4GlcNAcbeta1-6)Galbeta1-4GlcNAcbeta1-3Galbeta1-4GlcNAcbeta1-3Galbeta1-4Glcbeta-Cer(d18:1/20:0); Galalpha1-3Galbeta1-4GlcNAcbeta1-3(Galalpha1-3Galbeta1-4GlcNAcbeta1-6)Galbeta1-4GlcNAcbeta1-3Galbeta1-4GlcNAcbeta1-3Galbeta1-4Glcbeta-Cer(d18:1/22:0); Galalpha1-3Galbeta1-4GlcNAcbeta1-3(Galalpha1-3Galbeta1-4GlcNAcbeta1-6)Galbeta1-4GlcNAcbeta1-3Galbeta1-4GlcNAcbeta1-3Galbeta1-4Glcbeta-Cer(d18:1/24:0); Galalpha1-3Galbeta1-4GlcNAcbeta1-3(Galalpha1-3Galbeta1-4GlcNAcbeta1-6)Galbeta1-4GlcNAcbeta1-3Galbeta1-4GlcNAcbeta1-3Galbeta1-4Glcbeta-Cer(d18:1/24:1(15Z)); Galalpha1-3Galbeta1-4GlcNAcbeta1-3(Galalpha1-3Galbeta1-4GlcNAcbeta1-6)Galbeta1-4GlcNAcbeta1-3Galbeta1-4GlcNAcbeta1-3Galbeta1-4Glcbeta-Cer(d18:1/26:0); Galalpha1-3Galbeta1-4GlcNAcbeta1-3(Galalpha1-3Galbeta1-4GlcNAcbeta1-6)Galbeta1-4GlcNAcbeta1-3Galbeta1-4GlcNAcbeta1-3Galbeta1-4Glcbeta-Cer(d18:1/26:1(17Z)); Galalpha1-3Galbeta1-4GlcNAcbeta1-3(Galbeta1-4GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer; Galalpha1-3Galbeta1-4GlcNAcbeta1-3(Galbeta1-4GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/16:0); Galalpha1-3Galbeta1-4GlcNAcbeta1-3(Galbeta1-4GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/18:0); Galalpha1-3Galbeta1-4GlcNAcbeta1-3(Galbeta1-4GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/20:0); Galalpha1-3Galbeta1-4GlcNAcbeta1-3(Galbeta1-4GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/22:0); Galalpha1-3Galbeta1-4GlcNAcbeta1-3(Galbeta1-4GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:0); Galalpha1-3Galbeta1-4GlcNAcbeta1-3(Galbeta1-4GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:1(15Z)); Galalpha1-3Galbeta1-4GlcNAcbeta1-3(Galbeta1-4GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:0); Galalpha1-3Galbeta1-4GlcNAcbeta1-3(Galbeta1-4GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:1(17Z)); Galalpha1-3Galbeta1-4GlcNAcbeta1-3(Galbeta1-4GlcNAcbeta1-6)Galbeta1-4GlcNAcbeta1-3Galbeta1-4GlcNAcbeta1-3Galbeta1-4Glcbeta-Cer; Galalpha1-3Galbeta1-4GlcNAcbeta1-3(Galbeta1-4GlcNAcbeta1-6)Galbeta1-4GlcNAcbeta1-3Galbeta1-4GlcNAcbeta1-3Galbeta1-4Glcbeta-Cer(d18:1/16:0); Galalpha1-3Galbeta1-4GlcNAcbeta1-3(Galbeta1-4GlcNAcbeta1-6)Galbeta1-4GlcNAcbeta1-3Galbeta1-4GlcNAcbeta1-3Galbeta1-4Glcbeta-Cer(d18:1/18:0); Galalpha1-3Galbeta1-4GlcNAcbeta1-3(Galbeta1-4GlcNAcbeta1-6)Galbeta1-4GlcNAcbeta1-3Galbeta1-4GlcNAcbeta1-3Galbeta1-4Glcbeta-Cer(d18:1/20:0); Galalpha1-3Galbeta1-4GlcNAcbeta1-3(Galbeta1-4GlcNAcbeta1-6)Galbeta1-4GlcNAcbeta1-3Galbeta1-4GlcNAcbeta1-3Galbeta1-4Glcbeta-Cer(d18:1/22:0); Galalpha1-3Galbeta1-4GlcNAcbeta1-3(Galbeta1-4GlcNAcbeta1-6)Galbeta1-4GlcNAcbeta1-3Galbeta1-4GlcNAcbeta1-3Galbeta1-4Glcbeta-Cer(d18:1/24:0); Galalpha1-3Galbeta1-4GlcNAcbeta1-3(Galbeta1-4GlcNAcbeta1-6)Galbeta1-4GlcNAcbeta1-3Galbeta1-4GlcNAcbeta1-3Galbeta1-4Glcbeta-Cer(d18:1/24:1(15Z)); Galalpha1-3Galbeta1-4GlcNAcbeta1-3(Galbeta1-4GlcNAcbeta1-6)Galbeta1-4GlcNAcbeta1-3Galbeta1-4GlcNAcbeta1-3Galbeta1-4Glcbeta-Cer(d18:1/26:0); Galalpha1-3Galbeta1-4GlcNAcbeta1-3(Galbeta1-4GlcNAcbeta1-6)Galbeta1-4GlcNAcbeta1-3Galbeta1-4GlcNAcbeta1-3Galbeta1-4Glcbeta-Cer(d18:1/26:1(17Z)); Galalpha1-3Galbeta1-4GlcNAcbeta1-3(GalNAcbeta1-4)Galbeta1-4Glcbeta-Cer; Galalpha1-3Galbeta1-4GlcNAcbeta1-3(GalNAcbeta1-4)Galbeta1-4Glcbeta-Cer(d18:1/16:0); Galalpha1-3Galbeta1-4GlcNAcbeta1-3(GalNAcbeta1-4)Galbeta1-4Glcbeta-Cer(d18:1/18:0); Galalpha1-3Galbeta1-4GlcNAcbeta1-3(GalNAcbeta1-4)Galbeta1-4Glcbeta-Cer(d18:1/20:0); Galalpha1-3Galbeta1-4GlcNAcbeta1-3(GalNAcbeta1-4)Galbeta1-4Glcbeta-Cer(d18:1/22:0); Galalpha1-3Galbeta1-4GlcNAcbeta1-3(GalNAcbeta1-4)Galbeta1-4Glcbeta-Cer(d18:1/24:0); Galalpha1-3Galbeta1-4GlcNAcbeta1-3(GalNAcbeta1-4)Galbeta1-4Glcbeta-Cer(d18:1/24:1(15Z)); Galalpha1-3Galbeta1-4GlcNAcbeta1-3(GalNAcbeta1-4)Galbeta1-4Glcbeta-Cer(d18:1/26:0); Galalpha1-3Galbeta1-4GlcNAcbeta1-3(GalNAcbeta1-4)Galbeta1-4Glcbeta-Cer(d18:1/26:1(17Z)); Galalpha1-3Galbeta1-4GlcNAcbeta1-3(GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer; Galalpha1-3Galbeta1-4GlcNAcbeta1-3(GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/16:0); Galalpha1-3Galbeta1-4GlcNAcbeta1-3(GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/18:0); Galalpha1-3Galbeta1-4GlcNAcbeta1-3(GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/20:0); Galalpha1-3Galbeta1-4GlcNAcbeta1-3(GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/22:0); Galalpha1-3Galbeta1-4GlcNAcbeta1-3(GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:0); Galalpha1-3Galbeta1-4GlcNAcbeta1-3(GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:1(15Z)); Galalpha1-3Galbeta1-4GlcNAcbeta1-3(GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:0); Galalpha1-3Galbeta1-4GlcNAcbeta1-3(GlcNAcbeta1-6)Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:1(17Z)); Galalpha1-3Galbeta1-4GlcNAcbeta1-3(GlcNAcbeta1-6)Galbeta1-4GlcNAcbeta1-3Galbeta1-4GlcNAcbeta1-3Galbeta1-4Glcbeta-Cer; Galalpha1-3Galbeta1-4GlcNAcbeta1-3(GlcNAcbeta1-6)Galbeta1-4GlcNAcbeta1-3Galbeta1-4GlcNAcbeta1-3Galbeta1-4Glcbeta-Cer(d18:1/16:0); Galalpha1-3Galbeta1-4GlcNAcbeta1-3(GlcNAcbeta1-6)Galbeta1-4GlcNAcbeta1-3Galbeta1-4GlcNAcbeta1-3Galbeta1-4Glcbeta-Cer(d18:1/18:0); Galalpha1-3Galbeta1-4GlcNAcbeta1-3(GlcNAcbeta1-6)Galbeta1-4GlcNAcbeta1-3Galbeta1-4GlcNAcbeta1-3Galbeta1-4Glcbeta-Cer(d18:1/20:0); Galalpha1-3Galbeta1-4GlcNAcbeta1-3(GlcNAcbeta1-6)Galbeta1-4GlcNAcbeta1-3Galbeta1-4GlcNAcbeta1-3Galbeta1-4Glcbeta-Cer(d18:1/22:0); Galalpha1-3Galbeta1-4GlcNAcbeta1-3(GlcNAcbeta1-6)Galbeta1-4GlcNAcbeta1-3Galbeta1-4GlcNAcbeta1-3Galbeta1-4Glcbeta-Cer(d18:1/24:0); Galalpha1-3Galbeta1-4GlcNAcbeta1-3(GlcNAcbeta1-6)Galbeta1-4GlcNAcbeta1-3Galbeta1-4GlcNAcbeta1-3Galbeta1-4Glcbeta-Cer(d18:1/24:1(15Z)); Galalpha1-3Galbeta1-4GlcNAcbeta1-3(GlcNAcbeta1-6)Galbeta1-4GlcNAcbeta1-3Galbeta1-4GlcNAcbeta1-3Galbeta1-4Glcbeta-Cer(d18:1/26:0); Galalpha1-3Galbeta1-4GlcNAcbeta1-3(GlcNAcbeta1-6)Galbeta1-4GlcNAcbeta1-3Galbeta1-4GlcNAcbeta1-3Galbeta1-4Glcbeta-Cer(d18:1/26:1(17Z)); Galalpha1-3Galbeta1-4GlcNAcbeta1-3Galbeta1-4Glcbeta-Cer; Galalpha1-3Galbeta1-4GlcNAcbeta1-3Galbeta1-4Glcbeta-Cer(d18:1/16:0); Galalpha1-3Galbeta1-4GlcNAcbeta1-3Galbeta1-4Glcbeta-Cer(d18:1/18:0); Galalpha1-3Galbeta1-4GlcNAcbeta1-3Galbeta1-4Glcbeta-Cer(d18:1/20:0); Galalpha1-3Galbeta1-4GlcNAcbeta1-3Galbeta1-4Glcbeta-Cer(d18:1/22:0); Galalpha1-3Galbeta1-4GlcNAcbeta1-3Galbeta1-4Glcbeta-Cer(d18:1/24:0); Galalpha1-3Galbeta1-4GlcNAcbeta1-3Galbeta1-4Glcbeta-Cer(d18:1/24:1(15Z)); Galalpha1-3Galbeta1-4GlcNAcbeta1-3Galbeta1-4Glcbeta-Cer(d18:1/26:0); Galalpha1-3Galbeta1-4GlcNAcbeta1-3Galbeta1-4Glcbeta-Cer(d18:1/26:1(17Z)); Galalpha1-3Galbeta1-4GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer; Galalpha1-3Galbeta1-4GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/16:0); Galalpha1-3Galbeta1-4GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/18:0); Galalpha1-3Galbeta1-4GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/20:0); Galalpha1-3Galbeta1-4GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/22:0); Galalpha1-3Galbeta1-4GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:0); Galalpha1-3Galbeta1-4GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:1(15Z)); Galalpha1-3Galbeta1-4GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:0); Galalpha1-3Galbeta1-4GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:1(17Z)); Galalpha1-3Galbeta1-4GlcNAcbeta1-3Galbeta1-4GlcNAcbeta1-3Galbeta1-4Glcbeta-Cer; Galalpha1-3Galbeta1-4GlcNAcbeta1-3Galbeta1-4GlcNAcbeta1-3Galbeta1-4Glcbeta-Cer(d18:1/16:0); Galalpha1-3Galbeta1-4GlcNAcbeta1-3Galbeta1-4GlcNAcbeta1-3Galbeta1-4Glcbeta-Cer(d18:1/18:0); Galalpha1-3Galbeta1-4GlcNAcbeta1-3Galbeta1-4GlcNAcbeta1-3Galbeta1-4Glcbeta-Cer(d18:1/20:0); Galalpha1-3Galbeta1-4GlcNAcbeta1-3Galbeta1-4GlcNAcbeta1-3Galbeta1-4Glcbeta-Cer(d18:1/22:0); Galalpha1-3Galbeta1-4GlcNAcbeta1-3Galbeta1-4GlcNAcbeta1-3Galbeta1-4Glcbeta-Cer(d18:1/24:0); Galalpha1-3Galbeta1-4GlcNAcbeta1-3Galbeta1-4GlcNAcbeta1-3Galbeta1-4Glcbeta-Cer(d18:1/24:1(15Z)); Galalpha1-3Galbeta1-4GlcNAcbeta1-3Galbeta1-4GlcNAcbeta1-3Galbeta1-4Glcbeta-Cer(d18:1/26:0); Galalpha1-3Galbeta1-4GlcNAcbeta1-3Galbeta1-4GlcNAcbeta1-3Galbeta1-4Glcbeta-Cer(d18:1/26:1(17Z)); Galalpha1-3Galbeta1-4GlcNAcbeta1-3Galbeta1-4GlcNAcbeta1-3Galbeta1-4GlcNAcbeta1-3Galbeta1-4Glcbeta-Cer; Galalpha1-3Galbeta1-4GlcNAcbeta1-3Galbeta1-4GlcNAcbeta1-3Galbeta1-4GlcNAcbeta1-3Galbeta1-4Glcbeta-Cer(d18:1/16:0); Galalpha1-3Galbeta1-4GlcNAcbeta1-3Galbeta1-4GlcNAcbeta1-3Galbeta1-4GlcNAcbeta1-3Galbeta1-4Glcbeta-Cer(d18:1/18:0); Galalpha1-3Galbeta1-4GlcNAcbeta1-3Galbeta1-4GlcNAcbeta1-3Galbeta1-4GlcNAcbeta1-3Galbeta1-4Glcbeta-Cer(d18:1/20:0); Galalpha1-3Galbeta1-4GlcNAcbeta1-3Galbeta1-4GlcNAcbeta1-3Galbeta1-4GlcNAcbeta1-3Galbeta1-4Glcbeta-Cer(d18:1/22:0); Galalpha1-3Galbeta1-4GlcNAcbeta1-3Galbeta1-4GlcNAcbeta1-3Galbeta1-4GlcNAcbeta1-3Galbeta1-4Glcbeta-Cer(d18:1/24:0); Galalpha1-3Galbeta1-4GlcNAcbeta1-3Galbeta1-4GlcNAcbeta1-3Galbeta1-4GlcNAcbeta1-3Galbeta1-4Glcbeta-Cer(d18:1/24:1(15Z)); Galalpha1-3Galbeta1-4GlcNAcbeta1-3Galbeta1-4GlcNAcbeta1-3Galbeta1-4GlcNAcbeta1-3Galbeta1-4Glcbeta-Cer(d18:1/26:0); Galalpha1-3Galbeta1-4GlcNAcbeta1-3Galbeta1-4GlcNAcbeta1-3Galbeta1-4GlcNAcbeta1-3Galbeta1-4Glcbeta-Cer(d18:1/26:1(17Z)); Galalpha1-4Galbeta1-4Glcbeta-Cer; Galalpha1-4Galbeta1-4Glcbeta-Cer(d18:1/16:0); Galalpha1-4Galbeta1-4Glcbeta-Cer(d18:1/18:0); Galalpha1-4Galbeta1-4Glcbeta-Cer(d18:1/20:0); Galalpha1-4Galbeta1-4Glcbeta-Cer(d18:1/22:0); Galalpha1-4Galbeta1-4Glcbeta-Cer(d18:1/24:0); Galalpha1-4Galbeta1-4Glcbeta-Cer(d18:1/24:1(15Z)); Galalpha1-4Galbeta1-4Glcbeta-Cer(d18:1/26:0); Galalpha1-4Galbeta1-4Glcbeta-Cer(d18:1/26:1(17Z)); Galalpha1-4Galbeta1-4GlcNAcbeta1-3Galbeta1-4Glcbeta-Cer; Galalpha1-4Galbeta1-4GlcNAcbeta1-3Galbeta1-4Glcbeta-Cer(d18:1/16:0); Galalpha1-4Galbeta1-4GlcNAcbeta1-3Galbeta1-4Glcbeta-Cer(d18:1/18:0); Galalpha1-4Galbeta1-4GlcNAcbeta1-3Galbeta1-4Glcbeta-Cer(d18:1/20:0); Galalpha1-4Galbeta1-4GlcNAcbeta1-3Galbeta1-4Glcbeta-Cer(d18:1/22:0); Galalpha1-4Galbeta1-4GlcNAcbeta1-3Galbeta1-4Glcbeta-Cer(d18:1/24:0); Galalpha1-4Galbeta1-4GlcNAcbeta1-3Galbeta1-4Glcbeta-Cer(d18:1/24:1(15Z)); Galalpha1-4Galbeta1-4GlcNAcbeta1-3Galbeta1-4Glcbeta-Cer(d18:1/26:0); Galalpha1-4Galbeta1-4GlcNAcbeta1-3Galbeta1-4Glcbeta-Cer(d18:1/26:1(17Z)); Galalpha1-4Galbeta-Cer; Galalpha1-4Galbeta-Cer(d18:1/16:0); Galalpha1-4Galbeta-Cer(d18:1/18:0); Galalpha1-4Galbeta-Cer(d18:1/20:0); Galalpha1-4Galbeta-Cer(d18:1/22:0); Galalpha1-4Galbeta-Cer(d18:1/24:0); Galalpha1-4Galbeta-Cer(d18:1/24:1(15Z)); Galalpha1-4Galbeta-Cer(d18:1/26:0); Galalpha1-4Galbeta-Cer(d18:1/26:1(17Z)); Galalpha1-4GalNAcbeta1-3Galalpha1-4Galbeta1-4Glcbeta-Cer; Galalpha1-4GalNAcbeta1-3Galalpha1-4Galbeta1-4Glcbeta-Cer(d18:1/16:0); Galalpha1-4GalNAcbeta1-3Galalpha1-4Galbeta1-4Glcbeta-Cer(d18:1/18:0); Galalpha1-4GalNAcbeta1-3Galalpha1-4Galbeta1-4Glcbeta-Cer(d18:1/20:0); Galalpha1-4GalNAcbeta1-3Galalpha1-4Galbeta1-4Glcbeta-Cer(d18:1/22:0); Galalpha1-4GalNAcbeta1-3Galalpha1-4Galbeta1-4Glcbeta-Cer(d18:1/24:0); Galalpha1-4GalNAcbeta1-3Galalpha1-4Galbeta1-4Glcbeta-Cer(d18:1/24:1(15Z)); Galalpha1-4GalNAcbeta1-3Galalpha1-4Galbeta1-4Glcbeta-Cer(d18:1/26:0); Galalpha1-4GalNAcbeta1-3Galalpha1-4Galbeta1-4Glcbeta-Cer(d18:1/26:1(17Z)); Galbeta1-3(Fucalpha1-4)GlcNAcbeta1-3Galbeta1-3(Fucalpha1-4)GlcNAcbeta1-3Galbeta1-4Glcbeta-Cer; Galbeta1-3(Fucalpha1-4)GlcNAcbeta1-3Galbeta1-3(Fucalpha1-4)GlcNAcbeta1-3Galbeta1-4Glcbeta-Cer(d18:1/16:0); Galbeta1-3(Fucalpha1-4)GlcNAcbeta1-3Galbeta1-3(Fucalpha1-4)GlcNAcbeta1-3Galbeta1-4Glcbeta-Cer(d18:1/18:0); Galbeta1-3(Fucalpha1-4)GlcNAcbeta1-3Galbeta1-3(Fucalpha1-4)GlcNAcbeta1-3Galbeta1-4Glcbeta-Cer(d18:1/20:0); Galbeta1-3(Fucalpha1-4)GlcNAcbeta1-3Galbeta1-3(Fucalpha1-4)GlcNAcbeta1-3Galbeta1-4Glcbeta-Cer(d18:1/22:0); Galbeta1-3(Fucalpha1-4)GlcNAcbeta1-3Galbeta1-3(Fucalpha1-4)GlcNAcbeta1-3Galbeta1-4Glcbeta-Cer(d18:1/24:0); Galbeta1-3(Fucalpha1-4)GlcNAcbeta1-3Galbeta1-3(Fucalpha1-4)GlcNAcbeta1-3Galbeta1-4Glcbeta-Cer(d18:1/24:1(15Z)); Galbeta1-3(Fucalpha1-4)GlcNAcbeta1-3Galbeta1-3(Fucalpha1-4)GlcNAcbeta1-3Galbeta1-4Glcbeta-Cer(d18:1/26:0); Galbeta1-3(Fucalpha1-4)GlcNAcbeta1-3Galbeta1-3(Fucalpha1-4)GlcNAcbeta1-3Galbeta1-4Glcbeta-Cer(d18:1/26:1(17Z)); Galbeta1-3(Fucalpha1-4)GlcNAcbeta1-3Galbeta1-3GlcNAcbeta1-3Galbeta1-4Glcbeta-Cer; Galbeta1-3(Fucalpha1-4)GlcNAcbeta1-3Galbeta1-3GlcNAcbeta1-3Galbeta1-4Glcbeta-Cer(d18:1/16:0); Galbeta1-3(Fucalpha1-4)GlcNAcbeta1-3Galbeta1-3GlcNAcbeta1-3Galbeta1-4Glcbeta-Cer(d18:1/18:0); Galbeta1-3(Fucalpha1-4)GlcNAcbeta1-3Galbeta1-3GlcNAcbeta1-3Galbeta1-4Glcbeta-Cer(d18:1/20:0); Galbeta1-3(Fucalpha1-4)GlcNAcbeta1-3Galbeta1-3GlcNAcbeta1-3Galbeta1-4Glcbeta-Cer(d18:1/22:0); Galbeta1-3(Fucalpha1-4)GlcNAcbeta1-3Galbeta1-3GlcNAcbeta1-3Galbeta1-4Glcbeta-Cer(d18:1/24:0); Galbeta1-3(Fucalpha1-4)GlcNAcbeta1-3Galbeta1-3GlcNAcbeta1-3Galbeta1-4Glcbeta-Cer(d18:1/24:1(15Z)); Galbeta1-3(Fucalpha1-4)GlcNAcbeta1-3Galbeta1-3GlcNAcbeta1-3Galbeta1-4Glcbeta-Cer(d18:1/26:0); Galbeta1-3(Fucalpha1-4)GlcNAcbeta1-3Galbeta1-3GlcNAcbeta1-3Galbeta1-4Glcbeta-Cer(d18:1/26:1(17Z)); Galbeta1-3(Fucalpha1-4)GlcNAcbeta1-3Galbeta1-4(Fucalpha1-3)GlcNAcbeta1-3Galbeta1-4Glcbeta-Cer; Galbeta1-3(Fucalpha1-4)GlcNAcbeta1-3Galbeta1-4(Fucalpha1-3)GlcNAcbeta1-3Galbeta1-4Glcbeta-Cer(d18:1/16:0); Galbeta1-3(Fucalpha1-4)GlcNAcbeta1-3Galbeta1-4(Fucalpha1-3)GlcNAcbeta1-3Galbeta1-4Glcbeta-Cer(d18:1/18:0); Galbeta1-3(Fucalpha1-4)GlcNAcbeta1-3Galbeta1-4(Fucalpha1-3)GlcNAcbeta1-3Galbeta1-4Glcbeta-Cer(d18:1/20:0); Galbeta1-3(Fucalpha1-4)GlcNAcbeta1-3Galbeta1-4(Fucalpha1-3)GlcNAcbeta1-3Galbeta1-4Glcbeta-Cer(d18:1/22:0); Galbeta1-3(Fucalpha1-4)GlcNAcbeta1-3Galbeta1-4(Fucalpha1-3)GlcNAcbeta1-3Galbeta1-4Glcbeta-Cer(d18:1/24:0); Galbeta1-3(Fucalpha1-4)GlcNAcbeta1-3Galbeta1-4(Fucalpha1-3)GlcNAcbeta1-3Galbeta1-4Glcbeta-Cer(d18:1/24:1(15Z)); Galbeta1-3(Fucalpha1-4)GlcNAcbeta1-3Galbeta1-4(Fucalpha1-3)GlcNAcbeta1-3Galbeta1-4Glcbeta-Cer(d18:1/26:0); Galbeta1-3(Fucalpha1-4)GlcNAcbeta1-3Galbeta1-4(Fucalpha1-3)GlcNAcbeta1-3Galbeta1-4Glcbeta-Cer(d18:1/26:1(17Z)); Galbeta1-3(Fucalpha1-4)GlcNAcbeta1-3Galbeta1-4Glcbeta-Cer; Galbeta1-3(Fucalpha1-4)GlcNAcbeta1-3Galbeta1-4Glcbeta-Cer(d18:1/16:0); Galbeta1-3(Fucalpha1-4)GlcNAcbeta1-3Galbeta1-4Glcbeta-Cer(d18:1/18:0); Galbeta1-3(Fucalpha1-4)GlcNAcbeta1-3Galbeta1-4Glcbeta-Cer(d18:1/20:0); Galbeta1-3(Fucalpha1-4)GlcNAcbeta1-3Galbeta1-4Glcbeta-Cer(d18:1/22:0); Galbeta1-3(Fucalpha1-4)GlcNAcbeta1-3Galbeta1-4Glcbeta-Cer(d18:1/24:0); Galbeta1-3(Fucalpha1-4)GlcNAcbeta1-3Galbeta1-4Glcbeta-Cer(d18:1/24:1(15Z)); Galbeta1-3(Fucalpha1-4)GlcNAcbeta1-3Galbeta1-4Glcbeta-Cer(d18:1/26:0); Galbeta1-3(Fucalpha1-4)GlcNAcbeta1-3Galbeta1-4Glcbeta-Cer(d18:1/26:1(17Z)); Galbeta1-3(Fucalpha1-4)GlcNAcbeta1-3Galbeta1-4GlcNAcbeta1-3Galbeta1-4Glcbeta-Cer; Galbeta1-3(Fucalpha1-4)GlcNAcbeta1-3Galbeta1-4GlcNAcbeta1-3Galbeta1-4Glcbeta-Cer(d18:1/16:0); Galbeta1-3(Fucalpha1-4)GlcNAcbeta1-3Galbeta1-4GlcNAcbeta1-3Galbeta1-4Glcbeta-Cer(d18:1/18:0); Galbeta1-3(Fucalpha1-4)GlcNAcbeta1-3Galbeta1-4GlcNAcbeta1-3Galbeta1-4Glcbeta-Cer(d18:1/20:0); Galbeta1-3(Fucalpha1-4)GlcNAcbeta1-3Galbeta1-4GlcNAcbeta1-3Galbeta1-4Glcbeta-Cer(d18:1/22:0); Galbeta1-3(Fucalpha1-4)GlcNAcbeta1-3Galbeta1-4GlcNAcbeta1-3Galbeta1-4Glcbeta-Cer(d18:1/24:0); Galbeta1-3(Fucalpha1-4)GlcNAcbeta1-3Galbeta1-4GlcNAcbeta1-3Galbeta1-4Glcbeta-Cer(d18:1/24:1(15Z)); Galbeta1-3(Fucalpha1-4)GlcNAcbeta1-3Galbeta1-4GlcNAcbeta1-3Galbeta1-4Glcbeta-Cer(d18:1/26:0); Galbeta1-3(Fucalpha1-4)GlcNAcbeta1-3Galbeta1-4GlcNAcbeta1-3Galbeta1-4Glcbeta-Cer(d18:1/26:1(17Z)); Galbeta1-3(Fucalpha2-3GlcNAcbeta1-6)GalNAcbeta1-3Galalpha1-4Galbeta1-4Glcbeta-Cer; Galbeta1-3(Fucalpha2-3GlcNAcbeta1-6)GalNAcbeta1-3Galalpha1-4Galbeta1-4Glcbeta-Cer(d18:1/16:0); Galbeta1-3(Fucalpha2-3GlcNAcbeta1-6)GalNAcbeta1-3Galalpha1-4Galbeta1-4Glcbeta-Cer(d18:1/18:0); Galbeta1-3(Fucalpha2-3GlcNAcbeta1-6)GalNAcbeta1-3Galalpha1-4Galbeta1-4Glcbeta-Cer(d18:1/20:0); Galbeta1-3(Fucalpha2-3GlcNAcbeta1-6)GalNAcbeta1-3Galalpha1-4Galbeta1-4Glcbeta-Cer(d18:1/22:0); Galbeta1-3(Fucalpha2-3GlcNAcbeta1-6)GalNAcbeta1-3Galalpha1-4Galbeta1-4Glcbeta-Cer(d18:1/24:0); Galbeta1-3(Fucalpha2-3GlcNAcbeta1-6)GalNAcbeta1-3Galalpha1-4Galbeta1-4Glcbeta-Cer(d18:1/24:1(15Z)); Galbeta1-3(Fucalpha2-3GlcNAcbeta1-6)GalNAcbeta1-3Galalpha1-4Galbeta1-4Glcbeta-Cer(d18:1/26:0); Galbeta1-3(Fucalpha2-3GlcNAcbeta1-6)GalNAcbeta1-3Galalpha1-4Galbeta1-4Glcbeta-Cer(d18:1/26:1(17Z)); Galbeta1-3(Galbeta1-4GlcNAcbeta1-6)GalNAcbeta1-3Galalpha1-4Galbeta1-4Glcbeta-Cer; Galbeta1-3(Galbeta1-4GlcNAcbeta1-6)GalNAcbeta1-3Galalpha1-4Galbeta1-4Glcbeta-Cer(d18:1/16:0); Galbeta1-3(Galbeta1-4GlcNAcbeta1-6)GalNAcbeta1-3Galalpha1-4Galbeta1-4Glcbeta-Cer(d18:1/18:0); Galbeta1-3(Galbeta1-4GlcNAcbeta1-6)GalNAcbeta1-3Galalpha1-4Galbeta1-4Glcbeta-Cer(d18:1/20:0); Galbeta1-3(Galbeta1-4GlcNAcbeta1-6)GalNAcbeta1-3Galalpha1-4Galbeta1-4Glcbeta-Cer(d18:1/22:0); Galbeta1-3(Galbeta1-4GlcNAcbeta1-6)GalNAcbeta1-3Galalpha1-4Galbeta1-4Glcbeta-Cer(d18:1/24:0); Galbeta1-3(Galbeta1-4GlcNAcbeta1-6)GalNAcbeta1-3Galalpha1-4Galbeta1-4Glcbeta-Cer(d18:1/24:1(15Z)); Galbeta1-3(Galbeta1-4GlcNAcbeta1-6)GalNAcbeta1-3Galalpha1-4Galbeta1-4Glcbeta-Cer(d18:1/26:0); Galbeta1-3(Galbeta1-4GlcNAcbeta1-6)GalNAcbeta1-3Galalpha1-4Galbeta1-4Glcbeta-Cer(d18:1/26:1(17Z)); Galbeta1-3(GlcNAcbeta1-6)GalNAcbeta1-3Galalpha1-4Galbeta1-4Glcbeta-Cer; Galbeta1-3(GlcNAcbeta1-6)GalNAcbeta1-3Galalpha1-4Galbeta1-4Glcbeta-Cer(d18:1/16:0); Galbeta1-3(GlcNAcbeta1-6)GalNAcbeta1-3Galalpha1-4Galbeta1-4Glcbeta-Cer(d18:1/18:0); Galbeta1-3(GlcNAcbeta1-6)GalNAcbeta1-3Galalpha1-4Galbeta1-4Glcbeta-Cer(d18:1/20:0); Galbeta1-3(GlcNAcbeta1-6)GalNAcbeta1-3Galalpha1-4Galbeta1-4Glcbeta-Cer(d18:1/22:0); Galbeta1-3(GlcNAcbeta1-6)GalNAcbeta1-3Galalpha1-4Galbeta1-4Glcbeta-Cer(d18:1/24:0); Galbeta1-3(GlcNAcbeta1-6)GalNAcbeta1-3Galalpha1-4Galbeta1-4Glcbeta-Cer(d18:1/24:1(15Z)); Galbeta1-3(GlcNAcbeta1-6)GalNAcbeta1-3Galalpha1-4Galbeta1-4Glcbeta-Cer(d18:1/26:0); Galbeta1-3(GlcNAcbeta1-6)GalNAcbeta1-3Galalpha1-4Galbeta1-4Glcbeta-Cer(d18:1/26:1(17Z)); Galbeta1-3(NeuAcalpha2-6)(Fucalpha1-4)GlcNAcbeta1-3Galbeta1-4Glcbeta-Cer; Galbeta1-3(NeuAcalpha2-6)(Fucalpha1-4)GlcNAcbeta1-3Galbeta1-4Glcbeta-Cer(d18:1/16:0); Galbeta1-3(NeuAcalpha2-6)(Fucalpha1-4)GlcNAcbeta1-3Galbeta1-4Glcbeta-Cer(d18:1/18:0); Galbeta1-3(NeuAcalpha2-6)(Fucalpha1-4)GlcNAcbeta1-3Galbeta1-4Glcbeta-Cer(d18:1/20:0); Galbeta1-3(NeuAcalpha2-6)(Fucalpha1-4)GlcNAcbeta1-3Galbeta1-4Glcbeta-Cer(d18:1/22:0); Galbeta1-3(NeuAcalpha2-6)(Fucalpha1-4)GlcNAcbeta1-3Galbeta1-4Glcbeta-Cer(d18:1/24:0); Galbeta1-3(NeuAcalpha2-6)(Fucalpha1-4)GlcNAcbeta1-3Galbeta1-4Glcbeta-Cer(d18:1/24:1(15Z)); Galbeta1-3(NeuAcalpha2-6)(Fucalpha1-4)GlcNAcbeta1-3Galbeta1-4Glcbeta-Cer(d18:1/26:0); Galbeta1-3(NeuAcalpha2-6)(Fucalpha1-4)GlcNAcbeta1-3Galbeta1-4Glcbeta-Cer(d18:1/26:1(17Z)); Galbeta1-3(NeuAcalpha2-6)GalNAcbeta1-4(NeuAcalpha2-3)Galbeta1-4Glcbeta-Cer; Galbeta1-3(NeuAcalpha2-6)GalNAcbeta1-4(NeuAcalpha2-3)Galbeta1-4Glcbeta-Cer(d18:1/16:0); Galbeta1-3(NeuAcalpha2-6)GalNAcbeta1-4(NeuAcalpha2-3)Galbeta1-4Glcbeta-Cer(d18:1/18:0); Galbeta1-3(NeuAcalpha2-6)GalNAcbeta1-4(NeuAcalpha2-3)Galbeta1-4Glcbeta-Cer(d18:1/20:0); Galbeta1-3(NeuAcalpha2-6)GalNAcbeta1-4(NeuAcalpha2-3)Galbeta1-4Glcbeta-Cer(d18:1/22:0); Galbeta1-3(NeuAcalpha2-6)GalNAcbeta1-4(NeuAcalpha2-3)Galbeta1-4Glcbeta-Cer(d18:1/24:0); Galbeta1-3(NeuAcalpha2-6)GalNAcbeta1-4(NeuAcalpha2-3)Galbeta1-4Glcbeta-Cer(d18:1/24:1(15Z)); Galbeta1-3(NeuAcalpha2-6)GalNAcbeta1-4(NeuAcalpha2-3)Galbeta1-4Glcbeta-Cer(d18:1/26:0); Galbeta1-3(NeuAcalpha2-6)GalNAcbeta1-4(NeuAcalpha2-3)Galbeta1-4Glcbeta-Cer(d18:1/26:1(17Z)); Galbeta1-3(NeuAcalpha2-6)GalNAcbeta1-4(NeuGcalpha2-8NeuAcalpha2-3)Galbeta1-4Glcbeta-Cer; Galbeta1-3(NeuAcalpha2-6)GalNAcbeta1-4(NeuGcalpha2-8NeuAcalpha2-3)Galbeta1-4Glcbeta-Cer(d18:1/16:0); Galbeta1-3(NeuAcalpha2-6)GalNAcbeta1-4(NeuGcalpha2-8NeuAcalpha2-3)Galbeta1-4Glcbeta-Cer(d18:1/18:0); Galbeta1-3(NeuAcalpha2-6)GalNAcbeta1-4(NeuGcalpha2-8NeuAcalpha2-3)Galbeta1-4Glcbeta-Cer(d18:1/20:0); Galbeta1-3(NeuAcalpha2-6)GalNAcbeta1-4(NeuGcalpha2-8NeuAcalpha2-3)Galbeta1-4Glcbeta-Cer(d18:1/22:0); Galbeta1-3(NeuAcalpha2-6)GalNAcbeta1-4(NeuGcalpha2-8NeuAcalpha2-3)Galbeta1-4Glcbeta-Cer(d18:1/24:0); Galbeta1-3(NeuAcalpha2-6)GalNAcbeta1-4(NeuGcalpha2-8NeuAcalpha2-3)Galbeta1-4Glcbeta-Cer(d18:1/24:1(15Z)); Galbeta1-3(NeuAcalpha2-6)GalNAcbeta1-4(NeuGcalpha2-8NeuAcalpha2-3)Galbeta1-4Glcbeta-Cer(d18:1/26:0); Galbeta1-3(NeuAcalpha2-6)GalNAcbeta1-4(NeuGcalpha2-8NeuAcalpha2-3)Galbeta1-4Glcbeta-Cer(d18:1/26:1(17Z)); Galbeta1-3(NeuAcalpha2-6)GalNAcbeta1-4Galbeta1-4Glcbeta-Cer; Galbeta1-3(NeuAcalpha2-6)GalNAcbeta1-4Galbeta1-4Glcbeta-Cer(d18:1/16:0); Galbeta1-3(NeuAcalpha2-6)GalNAcbeta1-4Galbeta1-4Glcbeta-Cer(d18:1/18:0); Galbeta1-3(NeuAcalpha2-6)GalNAcbeta1-4Galbeta1-4Glcbeta-Cer(d18:1/20:0); Galbeta1-3(NeuAcalpha2-6)GalNAcbeta1-4Galbeta1-4Glcbeta-Cer(d18:1/22:0); Galbeta1-3(NeuAcalpha2-6)GalNAcbeta1-4Galbeta1-4Glcbeta-Cer(d18:1/24:0); Galbeta1-3(NeuAcalpha2-6)GalNAcbeta1-4Galbeta1-4Glcbeta-Cer(d18:1/24:1(15Z)); Galbeta1-3(NeuAcalpha2-6)GalNAcbeta1-4Galbeta1-4Glcbeta-Cer(d18:1/26:0); Galbeta1-3(NeuAcalpha2-6)GalNAcbeta1-4Galbeta1-4Glcbeta-Cer(d18:1/26:1(17Z)); Galbeta1-3(NeuAcalpha2-6)GlcNAcbeta1-3Galbeta1-4Glcbeta-Cer; Galbeta1-3(NeuAcalpha2-6)GlcNAcbeta1-3Galbeta1-4Glcbeta-Cer(d18:1/16:0); Galbeta1-3(NeuAcalpha2-6)GlcNAcbeta1-3Galbeta1-4Glcbeta-Cer(d18:1/18:0); Galbeta1-3(NeuAcalpha2-6)GlcNAcbeta1-3Galbeta1-4Glcbeta-Cer(d18:1/20:0); Galbeta1-3(NeuAcalpha2-6)GlcNAcbeta1-3Galbeta1-4Glcbeta-Cer(d18:1/22:0); Galbeta1-3(NeuAcalpha2-6)GlcNAcbeta1-3Galbeta1-4Glcbeta-Cer(d18:1/24:0); Galbeta1-3(NeuAcalpha2-6)GlcNAcbeta1-3Galbeta1-4Glcbeta-Cer(d18:1/24:1(15Z)); Galbeta1-3(NeuAcalpha2-6)GlcNAcbeta1-3Galbeta1-4Glcbeta-Cer(d18:1/26:0); Galbeta1-3(NeuAcalpha2-6)GlcNAcbeta1-3Galbeta1-4Glcbeta-Cer(d18:1/26:1(17Z)); Galbeta1-3(NeuAcalpha2-8NeuAcalpha2-6)GalNAcbeta1-4Galbeta1-4Glcbeta-Cer; Galbeta1-3(NeuAcalpha2-8NeuAcalpha2-6)GalNAcbeta1-4Galbeta1-4Glcbeta-Cer(d18:1/16:0); Galbeta1-3(NeuAcalpha2-8NeuAcalpha2-6)GalNAcbeta1-4Galbeta1-4Glcbeta-Cer(d18:1/18:0); Galbeta1-3(NeuAcalpha2-8NeuAcalpha2-6)GalNAcbeta1-4Galbeta1-4Glcbeta-Cer(d18:1/20:0); Galbeta1-3(NeuAcalpha2-8NeuAcalpha2-6)GalNAcbeta1-4Galbeta1-4Glcbeta-Cer(d18:1/22:0); Galbeta1-3(NeuAcalpha2-8NeuAcalpha2-6)GalNAcbeta1-4Galbeta1-4Glcbeta-Cer(d18:1/24:0); Galbeta1-3(NeuAcalpha2-8NeuAcalpha2-6)GalNAcbeta1-4Galbeta1-4Glcbeta-Cer(d18:1/24:1(15Z)); Galbeta1-3(NeuAcalpha2-8NeuAcalpha2-6)GalNAcbeta1-4Galbeta1-4Glcbeta-Cer(d18:1/26:0); Galbeta1-3(NeuAcalpha2-8NeuAcalpha2-6)GalNAcbeta1-4Galbeta1-4Glcbeta-Cer(d18:1/26:1(17Z)); Galbeta1-3Galalpha1-4Galbeta1-4Glcbeta-Cer; Galbeta1-3Galalpha1-4Galbeta1-4Glcbeta-Cer(d18:1/16:0); Galbeta1-3Galalpha1-4Galbeta1-4Glcbeta-Cer(d18:1/18:0); Galbeta1-3Galalpha1-4Galbeta1-4Glcbeta-Cer(d18:1/20:0); Galbeta1-3Galalpha1-4Galbeta1-4Glcbeta-Cer(d18:1/22:0); Galbeta1-3Galalpha1-4Galbeta1-4Glcbeta-Cer(d18:1/24:0); Galbeta1-3Galalpha1-4Galbeta1-4Glcbeta-Cer(d18:1/24:1(15Z)); Galbeta1-3Galalpha1-4Galbeta1-4Glcbeta-Cer(d18:1/26:0); Galbeta1-3Galalpha1-4Galbeta1-4Glcbeta-Cer(d18:1/26:1(17Z)); Galbeta1-3Galbeta1-3GlcNAcbeta1-3Galbeta1-4Glcbeta-Cer; Galbeta1-3Galbeta1-3GlcNAcbeta1-3Galbeta1-4Glcbeta-Cer(d18:1/16:0); Galbeta1-3Galbeta1-3GlcNAcbeta1-3Galbeta1-4Glcbeta-Cer(d18:1/18:0); Galbeta1-3Galbeta1-3GlcNAcbeta1-3Galbeta1-4Glcbeta-Cer(d18:1/20:0); Galbeta1-3Galbeta1-3GlcNAcbeta1-3Galbeta1-4Glcbeta-Cer(d18:1/22:0); Galbeta1-3Galbeta1-3GlcNAcbeta1-3Galbeta1-4Glcbeta-Cer(d18:1/24:0); Galbeta1-3Galbeta1-3GlcNAcbeta1-3Galbeta1-4Glcbeta-Cer(d18:1/24:1(15Z)); Galbeta1-3Galbeta1-3GlcNAcbeta1-3Galbeta1-4Glcbeta-Cer(d18:1/26:0); Galbeta1-3Galbeta1-3GlcNAcbeta1-3Galbeta1-4Glcbeta-Cer(d18:1/26:1(17Z)); Galbeta1-3Galbeta1-4GlcNAcbeta1-3Galbeta1-4Glcbeta-Cer; Galbeta1-3Galbeta1-4GlcNAcbeta1-3Galbeta1-4Glcbeta-Cer(d18:1/16:0); Galbeta1-3Galbeta1-4GlcNAcbeta1-3Galbeta1-4Glcbeta-Cer(d18:1/18:0); Galbeta1-3Galbeta1-4GlcNAcbeta1-3Galbeta1-4Glcbeta-Cer(d18:1/20:0); Galbeta1-3Galbeta1-4GlcNAcbeta1-3Galbeta1-4Glcbeta-Cer(d18:1/22:0); Galbeta1-3Galbeta1-4GlcNAcbeta1-3Galbeta1-4Glcbeta-Cer(d18:1/24:0); Galbeta1-3Galbeta1-4GlcNAcbeta1-3Galbeta1-4Glcbeta-Cer(d18:1/24:1(15Z)); Galbeta1-3Galbeta1-4GlcNAcbeta1-3Galbeta1-4Glcbeta-Cer(d18:1/26:0); Galbeta1-3Galbeta1-4GlcNAcbeta1-3Galbeta1-4Glcbeta-Cer(d18:1/26:1(17Z)); Galbeta1-3GalNAcalpha1-3(Fucalpha1-2)Galbeta1-4GlcNAcbeta1-3Galbeta1-4Glcbeta-Cer; Galbeta1-3GalNAcalpha1-3(Fucalpha1-2)Galbeta1-4GlcNAcbeta1-3Galbeta1-4Glcbeta-Cer(d18:1/16:0); Galbeta1-3GalNAcalpha1-3(Fucalpha1-2)Galbeta1-4GlcNAcbeta1-3Galbeta1-4Glcbeta-Cer(d18:1/18:0); Galbeta1-3GalNAcalpha1-3(Fucalpha1-2)Galbeta1-4GlcNAcbeta1-3Galbeta1-4Glcbeta-Cer(d18:1/20:0); Galbeta1-3GalNAcalpha1-3(Fucalpha1-2)Galbeta1-4GlcNAcbeta1-3Galbeta1-4Glcbeta-Cer(d18:1/22:0); Galbeta1-3GalNAcalpha1-3(Fucalpha1-2)Galbeta1-4GlcNAcbeta1-3Galbeta1-4Glcbeta-Cer(d18:1/24:0); Galbeta1-3GalNAcalpha1-3(Fucalpha1-2)Galbeta1-4GlcNAcbeta1-3Galbeta1-4Glcbeta-Cer(d18:1/24:1(15Z)); Galbeta1-3GalNAcalpha1-3(Fucalpha1-2)Galbeta1-4GlcNAcbeta1-3Galbeta1-4Glcbeta-Cer(d18:1/26:0); Galbeta1-3GalNAcalpha1-3(Fucalpha1-2)Galbeta1-4GlcNAcbeta1-3Galbeta1-4Glcbeta-Cer(d18:1/26:1(17Z)); Galbeta1-3GalNAcalpha1-3(Fucalpha1-2)Galbeta1-4GlcNAcbeta1-3Galbeta1-4GlcNAcbeta1-3Galbeta1-4Glcbeta-Cer; Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/16:0); Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/18:0); Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/20:0); Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/22:0); Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/24:0); Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/24:1(15Z)); Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/26:0); Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/26:1(17Z)); Galbeta1-3GalNAcbeta1-3Galalpha1-3Galbeta1-4Glcbeta-Cer; Galbeta1-3GalNAcbeta1-3Galalpha1-3Galbeta1-4Glcbeta-Cer; Galbeta1-3GalNAcbeta1-3Galalpha1-3Galbeta1-4Glcbeta-Cer(d18:1/16:0); Galbeta1-3GalNAcbeta1-3Galalpha1-3Galbeta1-4Glcbeta-Cer(d18:1/18:0); Galbeta1-3GalNAcbeta1-3Galalpha1-3Galbeta1-4Glcbeta-Cer(d18:1/20:0); Galbeta1-3GalNAcbeta1-3Galalpha1-3Galbeta1-4Glcbeta-Cer(d18:1/22:0); Galbeta1-3GalNAcbeta1-3Galalpha1-3Galbeta1-4Glcbeta-Cer(d18:1/24:0); Galbeta1-3GalNAcbeta1-3Galalpha1-3Galbeta1-4Glcbeta-Cer(d18:1/24:1(15Z)); Galbeta1-3GalNAcbeta1-3Galalpha1-3Galbeta1-4Glcbeta-Cer(d18:1/26:0); Galbeta1-3GalNAcbeta1-3Galalpha1-3Galbeta1-4Glcbeta-Cer(d18:1/26:1(17Z)); Galbeta1-3GalNAcbeta1-3Galalpha1-4Galbeta1-4Glcbeta-Cer; Galbeta1-3GalNAcbeta1-3Galalpha1-4Galbeta1-4Glcbeta-Cer(d18:1/16:0); Galbeta1-3GalNAcbeta1-3Galalpha1-4Galbeta1-4Glcbeta-Cer(d18:1/18:0); Galbeta1-3GalNAcbeta1-3Galalpha1-4Galbeta1-4Glcbeta-Cer(d18:1/20:0); Galbeta1-3GalNAcbeta1-3Galalpha1-4Galbeta1-4Glcbeta-Cer(d18:1/22:0); Galbeta1-3GalNAcbeta1-3Galalpha1-4Galbeta1-4Glcbeta-Cer(d18:1/24:0); Galbeta1-3GalNAcbeta1-3Galalpha1-4Galbeta1-4Glcbeta-Cer(d18:1/24:1(15Z)); Galbeta1-3GalNAcbeta1-3Galalpha1-4Galbeta1-4Glcbeta-Cer(d18:1/26:0); Galbeta1-3GalNAcbeta1-3Galalpha1-4Galbeta1-4Glcbeta-Cer(d18:1/26:1(17Z)); Galbeta1-3GalNAcbeta1-4(KDNalpha2-3)Galbeta1-4Glcbeta-Cer; Galbeta1-3GalNAcbeta1-4(KDNalpha2-3)Galbeta1-4Glcbeta-Cer(d18:1/16:0); Galbeta1-3GalNAcbeta1-4(KDNalpha2-3)Galbeta1-4Glcbeta-Cer(d18:1/18:0); Galbeta1-3GalNAcbeta1-4(KDNalpha2-3)Galbeta1-4Glcbeta-Cer(d18:1/20:0); Galbeta1-3GalNAcbeta1-4(KDNalpha2-3)Galbeta1-4Glcbeta-Cer(d18:1/22:0); Galbeta1-3GalNAcbeta1-4(KDNalpha2-3)Galbeta1-4Glcbeta-Cer(d18:1/24:0); Galbeta1-3GalNAcbeta1-4(KDNalpha2-3)Galbeta1-4Glcbeta-Cer(d18:1/24:1(15Z)); Galbeta1-3GalNAcbeta1-4(KDNalpha2-3)Galbeta1-4Glcbeta-Cer(d18:1/26:0); Galbeta1-3GalNAcbeta1-4(KDNalpha2-3)Galbeta1-4Glcbeta-Cer(d18:1/26:1(17Z)); Galbeta1-3GalNAcbeta1-4(NeuAcalpha2-3)Galbeta1-3GalNAcbeta1-4Galbeta1-4Glcbeta-Cer; Galbeta1-3GalNAcbeta1-4(NeuAcalpha2-3)Galbeta1-3GalNAcbeta1-4Galbeta1-4Glcbeta-Cer(d18:1/16:0); Galbeta1-3GalNAcbeta1-4(NeuAcalpha2-3)Galbeta1-3GalNAcbeta1-4Galbeta1-4Glcbeta-Cer(d18:1/18:0); Galbeta1-3GalNAcbeta1-4(NeuAcalpha2-3)Galbeta1-3GalNAcbeta1-4Galbeta1-4Glcbeta-Cer(d18:1/20:0); Galbeta1-3GalNAcbeta1-4(NeuAcalpha2-3)Galbeta1-3GalNAcbeta1-4Galbeta1-4Glcbeta-Cer(d18:1/22:0); Galbeta1-3GalNAcbeta1-4(NeuAcalpha2-3)Galbeta1-3GalNAcbeta1-4Galbeta1-4Glcbeta-Cer(d18:1/24:0); Galbeta1-3GalNAcbeta1-4(NeuAcalpha2-3)Galbeta1-3GalNAcbeta1-4Galbeta1-4Glcbeta-Cer(d18:1/24:1(15Z)); Galbeta1-3GalNAcbeta1-4(NeuAcalpha2-3)Galbeta1-3GalNAcbeta1-4Galbeta1-4Glcbeta-Cer(d18:1/26:0); Galbeta1-3GalNAcbeta1-4(NeuAcalpha2-3)Galbeta1-3GalNAcbeta1-4Galbeta1-4Glcbeta-Cer(d18:1/26:1(17Z)); Galbeta1-3GalNAcbeta1-4(NeuAcalpha2-3)Galbeta1-4Glcbeta-Cer; Galbeta1-3GalNAcbeta1-4(NeuAcalpha2-3)Galbeta1-4Glcbeta-Cer(d18:1/12:0); Galbeta1-3GalNAcbeta1-4(NeuAcalpha2-3)Galbeta1-4Glcbeta-Cer(d18:1/14:0); Galbeta1-3GalNAcbeta1-4(NeuAcalpha2-3)Galbeta1-4Glcbeta-Cer(d18:1/16:0); Galbeta1-3GalNAcbeta1-4(NeuAcalpha2-3)Galbeta1-4Glcbeta-Cer(d18:1/18:0); Galbeta1-3GalNAcbeta1-4(NeuAcalpha2-3)Galbeta1-4Glcbeta-Cer(d18:1/20:0); Galbeta1-3GalNAcbeta1-4(NeuAcalpha2-3)Galbeta1-4Glcbeta-Cer(d18:1/22:0); Galbeta1-3GalNAcbeta1-4(NeuAcalpha2-3)Galbeta1-4Glcbeta-Cer(d18:1/24:0); Galbeta1-3GalNAcbeta1-4(NeuAcalpha2-3)Galbeta1-4Glcbeta-Cer(d18:1/24:1(15Z)); Galbeta1-3GalNAcbeta1-4(NeuAcalpha2-3)Galbeta1-4Glcbeta-Cer(d18:1/26:0); Galbeta1-3GalNAcbeta1-4(NeuAcalpha2-3)Galbeta1-4Glcbeta-Cer(d18:1/26:1(17Z)); Galbeta1-3GalNAcbeta1-4(NeuAcalpha2-8NeuAcalpha2-3)Galbeta1-4Glcbeta-Cer; Galbeta1-3GalNAcbeta1-4(NeuAcalpha2-8NeuAcalpha2-3)Galbeta1-4Glcbeta-Cer(d18:1/16:0); Galbeta1-3GalNAcbeta1-4(NeuAcalpha2-8NeuAcalpha2-3)Galbeta1-4Glcbeta-Cer(d18:1/18:0); Galbeta1-3GalNAcbeta1-4(NeuAcalpha2-8NeuAcalpha2-3)Galbeta1-4Glcbeta-Cer(d18:1/20:0); Galbeta1-3GalNAcbeta1-4(NeuAcalpha2-8NeuAcalpha2-3)Galbeta1-4Glcbeta-Cer(d18:1/22:0); Galbeta1-3GalNAcbeta1-4(NeuAcalpha2-8NeuAcalpha2-3)Galbeta1-4Glcbeta-Cer(d18:1/24:0); Galbeta1-3GalNAcbeta1-4(NeuAcalpha2-8NeuAcalpha2-3)Galbeta1-4Glcbeta-Cer(d18:1/24:1(15Z)); Galbeta1-3GalNAcbeta1-4(NeuAcalpha2-8NeuAcalpha2-3)Galbeta1-4Glcbeta-Cer(d18:1/26:0); Galbeta1-3GalNAcbeta1-4(NeuAcalpha2-8NeuAcalpha2-3)Galbeta1-4Glcbeta-Cer(d18:1/26:1(17Z)); Galbeta1-3GalNAcbeta1-4(NeuAcalpha2-8NeuAcalpha2-8NeuAcalpha2-3)Galbeta1-4Glcbeta-Cer; Galbeta1-3GalNAcbeta1-4(NeuAcalpha2-8NeuAcalpha2-8NeuAcalpha2-3)Galbeta1-4Glcbeta-Cer(d18:1/16:0); Galbeta1-3GalNAcbeta1-4(NeuAcalpha2-8NeuAcalpha2-8NeuAcalpha2-3)Galbeta1-4Glcbeta-Cer(d18:1/18:0); Galbeta1-3GalNAcbeta1-4(NeuAcalpha2-8NeuAcalpha2-8NeuAcalpha2-3)Galbeta1-4Glcbeta-Cer(d18:1/20:0); Galbeta1-3GalNAcbeta1-4(NeuAcalpha2-8NeuAcalpha2-8NeuAcalpha2-3)Galbeta1-4Glcbeta-Cer(d18:1/22:0); Galbeta1-3GalNAcbeta1-4(NeuAcalpha2-8NeuAcalpha2-8NeuAcalpha2-3)Galbeta1-4Glcbeta-Cer(d18:1/24:0); Galbeta1-3GalNAcbeta1-4(NeuAcalpha2-8NeuAcalpha2-8NeuAcalpha2-3)Galbeta1-4Glcbeta-Cer(d18:1/24:1(15Z)); Galbeta1-3GalNAcbeta1-4(NeuAcalpha2-8NeuAcalpha2-8NeuAcalpha2-3)Galbeta1-4Glcbeta-Cer(d18:1/26:0); Galbeta1-3GalNAcbeta1-4(NeuAcalpha2-8NeuAcalpha2-8NeuAcalpha2-3)Galbeta1-4Glcbeta-Cer(d18:1/26:1(17Z)); Galbeta1-3GalNAcbeta1-4(NeuGcalpha2-3)Galbeta1-3GalNAcbeta1-4Galbeta1-4Glcbeta-Cer; Galbeta1-3GalNAcbeta1-4(NeuGcalpha2-3)Galbeta1-3GalNAcbeta1-4Galbeta1-4Glcbeta-Cer(d18:1/16:0); Galbeta1-3GalNAcbeta1-4(NeuGcalpha2-3)Galbeta1-3GalNAcbeta1-4Galbeta1-4Glcbeta-Cer(d18:1/18:0); Galbeta1-3GalNAcbeta1-4(NeuGcalpha2-3)Galbeta1-3GalNAcbeta1-4Galbeta1-4Glcbeta-Cer(d18:1/20:0); Galbeta1-3GalNAcbeta1-4(NeuGcalpha2-3)Galbeta1-3GalNAcbeta1-4Galbeta1-4Glcbeta-Cer(d18:1/22:0); Galbeta1-3GalNAcbeta1-4(NeuGcalpha2-3)Galbeta1-3GalNAcbeta1-4Galbeta1-4Glcbeta-Cer(d18:1/24:0); Galbeta1-3GalNAcbeta1-4(NeuGcalpha2-3)Galbeta1-3GalNAcbeta1-4Galbeta1-4Glcbeta-Cer(d18:1/24:1(15Z)); Galbeta1-3GalNAcbeta1-4(NeuGcalpha2-3)Galbeta1-3GalNAcbeta1-4Galbeta1-4Glcbeta-Cer(d18:1/26:0); Galbeta1-3GalNAcbeta1-4(NeuGcalpha2-3)Galbeta1-3GalNAcbeta1-4Galbeta1-4Glcbeta-Cer(d18:1/26:1(17Z)); Galbeta1-3GalNAcbeta1-4(NeuGcalpha2-3)Galbeta1-4Glcbeta-Cer; Galbeta1-3GalNAcbeta1-4(NeuGcalpha2-3)Galbeta1-4Glcbeta-Cer(d18:1/16:0); Galbeta1-3GalNAcbeta1-4(NeuGcalpha2-3)Galbeta1-4Glcbeta-Cer(d18:1/18:0); Galbeta1-3GalNAcbeta1-4(NeuGcalpha2-3)Galbeta1-4Glcbeta-Cer(d18:1/20:0); Galbeta1-3GalNAcbeta1-4(NeuGcalpha2-3)Galbeta1-4Glcbeta-Cer(d18:1/22:0); Galbeta1-3GalNAcbeta1-4(NeuGcalpha2-3)Galbeta1-4Glcbeta-Cer(d18:1/24:0); Galbeta1-3GalNAcbeta1-4(NeuGcalpha2-3)Galbeta1-4Glcbeta-Cer(d18:1/24:1(15Z)); Galbeta1-3GalNAcbeta1-4(NeuGcalpha2-3)Galbeta1-4Glcbeta-Cer(d18:1/26:0); Galbeta1-3GalNAcbeta1-4(NeuGcalpha2-3)Galbeta1-4Glcbeta-Cer(d18:1/26:1(17Z)); Galbeta1-3GalNAcbeta1-4Galalpha1-4Galbeta1-4Glcbeta-Cer; Galbeta1-3GalNAcbeta1-4Galalpha1-4Galbeta1-4Glcbeta-Cer(d18:1/16:0); Galbeta1-3GalNAcbeta1-4Galalpha1-4Galbeta1-4Glcbeta-Cer(d18:1/18:0); Galbeta1-3GalNAcbeta1-4Galalpha1-4Galbeta1-4Glcbeta-Cer(d18:1/20:0); Galbeta1-3GalNAcbeta1-4Galalpha1-4Galbeta1-4Glcbeta-Cer(d18:1/22:0); Galbeta1-3GalNAcbeta1-4Galalpha1-4Galbeta1-4Glcbeta-Cer(d18:1/24:0); Galbeta1-3GalNAcbeta1-4Galalpha1-4Galbeta1-4Glcbeta-Cer(d18:1/24:1(15Z)); Galbeta1-3GalNAcbeta1-4Galalpha1-4Galbeta1-4Glcbeta-Cer(d18:1/26:0); Galbeta1-3GalNAcbeta1-4Galalpha1-4Galbeta1-4Glcbeta-Cer(d18:1/26:1(17Z)); Galbeta1-3GalNAcbeta1-4Galbeta1-4Glcbeta-Cer; Galbeta1-3GalNAcbeta1-4Galbeta1-4Glcbeta-Cer(d18:1/16:0); Galbeta1-3GalNAcbeta1-4Galbeta1-4Glcbeta-Cer(d18:1/18:0); Galbeta1-3GalNAcbeta1-4Galbeta1-4Glcbeta-Cer(d18:1/20:0); Galbeta1-3GalNAcbeta1-4Galbeta1-4Glcbeta-Cer(d18:1/22:0); Galbeta1-3GalNAcbeta1-4Galbeta1-4Glcbeta-Cer(d18:1/24:0); Galbeta1-3GalNAcbeta1-4Galbeta1-4Glcbeta-Cer(d18:1/24:1(15Z)); Galbeta1-3GalNAcbeta1-4Galbeta1-4Glcbeta-Cer(d18:1/26:0); Galbeta1-3GalNAcbeta1-4Galbeta1-4Glcbeta-Cer(d18:1/26:1(17Z)); Galbeta1-3GalNAcbeta1-4HSO3-3Galbeta1-4Glcbeta-Cer; Galbeta1-3GlcNAcbeta1-3Galbeta1-3GlcNAcbeta1-3Galbeta1-4Glcbeta-Cer; Galbeta1-3GlcNAcbeta1-3Galbeta1-3GlcNAcbeta1-3Galbeta1-4Glcbeta-Cer(d18:1/16:0); Galbeta1-3GlcNAcbeta1-3Galbeta1-3GlcNAcbeta1-3Galbeta1-4Glcbeta-Cer(d18:1/18:0); Galbeta1-3GlcNAcbeta1-3Galbeta1-3GlcNAcbeta1-3Galbeta1-4Glcbeta-Cer(d18:1/20:0); Galbeta1-3GlcNAcbeta1-3Galbeta1-3GlcNAcbeta1-3Galbeta1-4Glcbeta-Cer(d18:1/22:0); Galbeta1-3GlcNAcbeta1-3Galbeta1-3GlcNAcbeta1-3Galbeta1-4Glcbeta-Cer(d18:1/24:0); Galbeta1-3GlcNAcbeta1-3Galbeta1-3GlcNAcbeta1-3Galbeta1-4Glcbeta-Cer(d18:1/24:1(15Z)); Galbeta1-3GlcNAcbeta1-3Galbeta1-3GlcNAcbeta1-3Galbeta1-4Glcbeta-Cer(d18:1/26:0); Galbeta1-3GlcNAcbeta1-3Galbeta1-3GlcNAcbeta1-3Galbeta1-4Glcbeta-Cer(d18:1/26:1(17Z)); Galbeta1-3GlcNAcbeta1-3Galbeta1-4(Fucalpha1-3)GlcNAcbeta1-3Galbeta1-4Glcbeta-Cer; Galbeta1-3GlcNAcbeta1-3Galbeta1-4(Fucalpha1-3)GlcNAcbeta1-3Galbeta1-4Glcbeta-Cer(d18:1/16:0); Galbeta1-3GlcNAcbeta1-3Galbeta1-4(Fucalpha1-3)GlcNAcbeta1-3Galbeta1-4Glcbeta-Cer(d18:1/18:0); Galbeta1-3GlcNAcbeta1-3Galbeta1-4(Fucalpha1-3)GlcNAcbeta1-3Galbeta1-4Glcbeta-Cer(d18:1/20:0); Galbeta1-3GlcNAcbeta1-3Galbeta1-4(Fucalpha1-3)GlcNAcbeta1-3Galbeta1-4Glcbeta-Cer(d18:1/22:0); Galbeta1-3GlcNAcbeta1-3Galbeta1-4(Fucalpha1-3)GlcNAcbeta1-3Galbeta1-4Glcbeta-Cer(d18:1/24:0); Galbeta1-3GlcNAcbeta1-3Galbeta1-4(Fucalpha1-3)GlcNAcbeta1-3Galbeta1-4Glcbeta-Cer(d18:1/24:1(15Z)); Galbeta1-3GlcNAcbeta1-3Galbeta1-4(Fucalpha1-3)GlcNAcbeta1-3Galbeta1-4Glcbeta-Cer(d18:1/26:0); Galbeta1-3GlcNAcbeta1-3Galbeta1-4(Fucalpha1-3)GlcNAcbeta1-3Galbeta1-4Glcbeta-Cer(d18:1/26:1(17Z)); Galbeta1-3GlcNAcbeta1-3Galbeta1-4Glcbeta-Cer; Galbeta1-3GlcNAcbeta1-3Galbeta1-4Glcbeta-Cer(d18:1/16:0); Galbeta1-3GlcNAcbeta1-3Galbeta1-4Glcbeta-Cer(d18:1/18:0); Galbeta1-3GlcNAcbeta1-3Galbeta1-4Glcbeta-Cer(d18:1/20:0); Galbeta1-3GlcNAcbeta1-3Galbeta1-4Glcbeta-Cer(d18:1/22:0); Galbeta1-3GlcNAcbeta1-3Galbeta1-4Glcbeta-Cer(d18:1/24:0); Galbeta1-3GlcNAcbeta1-3Galbeta1-4Glcbeta-Cer(d18:1/24:1(15Z)); Galbeta1-3GlcNAcbeta1-3Galbeta1-4Glcbeta-Cer(d18:1/26:0); Galbeta1-3GlcNAcbeta1-3Galbeta1-4Glcbeta-Cer(d18:1/26:1(17Z)); Galbeta1-3GlcNAcbeta1-3Galbeta1-4GlcNAcbeta1-3Galbeta1-4Glcbeta-Cer; Galbeta1-3GlcNAcbeta1-3Galbeta1-4GlcNAcbeta1-3Galbeta1-4Glcbeta-Cer(d18:1/16:0); Galbeta1-3GlcNAcbeta1-3Galbeta1-4GlcNAcbeta1-3Galbeta1-4Glcbeta-Cer(d18:1/18:0); Galbeta1-3GlcNAcbeta1-3Galbeta1-4GlcNAcbeta1-3Galbeta1-4Glcbeta-Cer(d18:1/20:0); Galbeta1-3GlcNAcbeta1-3Galbeta1-4GlcNAcbeta1-3Galbeta1-4Glcbeta-Cer(d18:1/22:0); Galbeta1-3GlcNAcbeta1-3Galbeta1-4GlcNAcbeta1-3Galbeta1-4Glcbeta-Cer(d18:1/24:0); Galbeta1-3GlcNAcbeta1-3Galbeta1-4GlcNAcbeta1-3Galbeta1-4Glcbeta-Cer(d18:1/24:1(15Z)); Galbeta1-3GlcNAcbeta1-3Galbeta1-4GlcNAcbeta1-3Galbeta1-4Glcbeta-Cer(d18:1/26:0); Galbeta1-3GlcNAcbeta1-3Galbeta1-4GlcNAcbeta1-3Galbeta1-4Glcbeta-Cer(d18:1/26:1(17Z)); Galbeta1-4(Fucalpha1-3)GlcNAcbeta1-3Galalpha1-3(GalNAcbeta1-4)Galbeta1-4Glcbeta-Cer; Galbeta1-4(Fucalpha1-3)GlcNAcbeta1-3Galalpha1-3(GalNAcbeta1-4)Galbeta1-4Glcbeta-Cer(d18:1/16:0); Galbeta1-4(Fucalpha1-3)GlcNAcbeta1-3Galalpha1-3(GalNAcbeta1-4)Galbeta1-4Glcbeta-Cer(d18:1/18:0); Galbeta1-4(Fucalpha1-3)GlcNAcbeta1-3Galalpha1-3(GalNAcbeta1-4)Galbeta1-4Glcbeta-Cer(d18:1/20:0); Galbeta1-4(Fucalpha1-3)GlcNAcbeta1-3Galalpha1-3(GalNAcbeta1-4)Galbeta1-4Glcbeta-Cer(d18:1/22:0); Galbeta1-4(Fucalpha1-3)GlcNAcbeta1-3Galalpha1-3(GalNAcbeta1-4)Galbeta1-4Glcbeta-Cer(d18:1/24:0); Galbeta1-4(Fucalpha1-3)GlcNAcbeta1-3Galalpha1-3(GalNAcbeta1-4)Galbeta1-4Glcbeta-Cer(d18:1/24:1(15Z)); Galbeta1-4(Fucalpha1-3)GlcNAcbeta1-3Galalpha1-3(GalNAcbeta1-4)Galbeta1-4Glcbeta-Cer(d18:1/26:0); Galbeta1-4(Fucalpha1-3)GlcNAcbeta1-3Galalpha1-3(GalNAcbeta1-4)Galbeta1-4Glcbeta-Cer(d18:1/26:1(17Z)); Galbeta1-4(Fucalpha1-3)GlcNAcbeta1-3Galalpha1-3Galbeta1-4Glcbeta-Cer; Galbeta1-4(Fucalpha1-3)GlcNAcbeta1-3Galalpha1-3Galbeta1-4Glcbeta-Cer(d18:1/16:0); Galbeta1-4(Fucalpha1-3)GlcNAcbeta1-3Galalpha1-3Galbeta1-4Glcbeta-Cer(d18:1/18:0); Galbeta1-4(Fucalpha1-3)GlcNAcbeta1-3Galalpha1-3Galbeta1-4Glcbeta-Cer(d18:1/20:0); Galbeta1-4(Fucalpha1-3)GlcNAcbeta1-3Galalpha1-3Galbeta1-4Glcbeta-Cer(d18:1/22:0); Galbeta1-4(Fucalpha1-3)GlcNAcbeta1-3Galalpha1-3Galbeta1-4Glcbeta-Cer(d18:1/24:0); Galbeta1-4(Fucalpha1-3)GlcNAcbeta1-3Galalpha1-3Galbeta1-4Glcbeta-Cer(d18:1/24:1(15Z)); Galbeta1-4(Fucalpha1-3)GlcNAcbeta1-3Galalpha1-3Galbeta1-4Glcbeta-Cer(d18:1/26:0); Galbeta1-4(Fucalpha1-3)GlcNAcbeta1-3Galalpha1-3Galbeta1-4Glcbeta-Cer(d18:1/26:1(17Z)); Galbeta1-4(Fucalpha1-3)GlcNAcbeta1-3Galbeta1-3(Fucalpha1-4)GlcNAcbeta1-3Galbeta1-4Glcbeta-Cer; Galbeta1-4(Fucalpha1-3)GlcNAcbeta1-3Galbeta1-3(Fucalpha1-4)GlcNAcbeta1-3Galbeta1-4Glcbeta-Cer(d18:1/16:0); Galbeta1-4(Fucalpha1-3)GlcNAcbeta1-3Galbeta1-3(Fucalpha1-4)GlcNAcbeta1-3Galbeta1-4Glcbeta-Cer(d18:1/18:0); Galbeta1-4(Fucalpha1-3)GlcNAcbeta1-3Galbeta1-3(Fucalpha1-4)GlcNAcbeta1-3Galbeta1-4Glcbeta-Cer(d18:1/20:0); Galbeta1-4(Fucalpha1-3)GlcNAcbeta1-3Galbeta1-3(Fucalpha1-4)GlcNAcbeta1-3Galbeta1-4Glcbeta-Cer(d18:1/22:0); Galbeta1-4(Fucalpha1-3)GlcNAcbeta1-3Galbeta1-3(Fucalpha1-4)GlcNAcbeta1-3Galbeta1-4Glcbeta-Cer(d18:1/24:0); Galbeta1-4(Fucalpha1-3)GlcNAcbeta1-3Galbeta1-3(Fucalpha1-4)GlcNAcbeta1-3Galbeta1-4Glcbeta-Cer(d18:1/24:1(15Z)); Galbeta1-4(Fucalpha1-3)GlcNAcbeta1-3Galbeta1-3(Fucalpha1-4)GlcNAcbeta1-3Galbeta1-4Glcbeta-Cer(d18:1/26:0); Galbeta1-4(Fucalpha1-3)GlcNAcbeta1-3Galbeta1-3(Fucalpha1-4)GlcNAcbeta1-3Galbeta1-4Glcbeta-Cer(d18:1/26:1(17Z)); Galbeta1-4(Fucalpha1-3)GlcNAcbeta1-3Galbeta1-3GalNAcbeta1-4(NeuAcalpha2-3)Galbeta1-4Glcbeta-Cer; Galbeta1-4(Fucalpha1-3)GlcNAcbeta1-3Galbeta1-3GalNAcbeta1-4(NeuAcalpha2-3)Galbeta1-4Glcbeta-Cer(d18:1/16:0); Galbeta1-4(Fucalpha1-3)GlcNAcbeta1-3Galbeta1-3GalNAcbeta1-4(NeuAcalpha2-3)Galbeta1-4Glcbeta-Cer(d18:1/18:0); Galbeta1-4(Fucalpha1-3)GlcNAcbeta1-3Galbeta1-3GalNAcbeta1-4(NeuAcalpha2-3)Galbeta1-4Glcbeta-Cer(d18:1/20:0); Galbeta1-4(Fucalpha1-3)GlcNAcbeta1-3Galbeta1-3GalNAcbeta1-4(NeuAcalpha2-3)Galbeta1-4Glcbeta-Cer(d18:1/22:0); Galbeta1-4(Fucalpha1-3)GlcNAcbeta1-3Galbeta1-3GalNAcbeta1-4(NeuAcalpha2-3)Galbeta1-4Glcbeta-Cer(d18:1/24:0); Galbeta1-4(Fucalpha1-3)GlcNAcbeta1-3Galbeta1-3GalNAcbeta1-4(NeuAcalpha2-3)Galbeta1-4Glcbeta-Cer(d18:1/24:1(15Z)); Galbeta1-4(Fucalpha1-3)GlcNAcbeta1-3Galbeta1-3GalNAcbeta1-4(NeuAcalpha2-3)Galbeta1-4Glcbeta-Cer(d18:1/26:0); Galbeta1-4(Fucalpha1-3)GlcNAcbeta1-3Galbeta1-3GalNAcbeta1-4(NeuAcalpha2-3)Galbeta1-4Glcbeta-Cer(d18:1/26:1(17Z)); Galbeta1-4(Fucalpha1-3)GlcNAcbeta1-3Galbeta1-3GalNAcbeta1-4Galbeta1-4Glcbeta-Cer; Galbeta1-4(Fucalpha1-3)GlcNAcbeta1-3Galbeta1-3GalNAcbeta1-4Galbeta1-4Glcbeta-Cer(d18:1/16:0); Galbeta1-4(Fucalpha1-3)GlcNAcbeta1-3Galbeta1-3GalNAcbeta1-4Galbeta1-4Glcbeta-Cer(d18:1/18:0); Galbeta1-4(Fucalpha1-3)GlcNAcbeta1-3Galbeta1-3GalNAcbeta1-4Galbeta1-4Glcbeta-Cer(d18:1/20:0); Galbeta1-4(Fucalpha1-3)GlcNAcbeta1-3Galbeta1-3GalNAcbeta1-4Galbeta1-4Glcbeta-Cer(d18:1/22:0); Galbeta1-4(Fucalpha1-3)GlcNAcbeta1-3Galbeta1-3GalNAcbeta1-4Galbeta1-4Glcbeta-Cer(d18:1/24:0); Galbeta1-4(Fucalpha1-3)GlcNAcbeta1-3Galbeta1-3GalNAcbeta1-4Galbeta1-4Glcbeta-Cer(d18:1/24:1(15Z)); Galbeta1-4(Fucalpha1-3)GlcNAcbeta1-3Galbeta1-3GalNAcbeta1-4Galbeta1-4Glcbeta-Cer(d18:1/26:0); Galbeta1-4(Fucalpha1-3)GlcNAcbeta1-3Galbeta1-3GalNAcbeta1-4Galbeta1-4Glcbeta-Cer(d18:1/26:1(17Z)); Galbeta1-4(Fucalpha1-3)GlcNAcbeta1-3Galbeta1-4(Fucalpha1-3)GlcNAcbeta1-3Galbeta1-4(Fucalpha1-3)GlcNAcbeta1-3Galbeta1-4Glcbeta-Cer; Galbeta1-4(Fucalpha1-3)GlcNAcbeta1-3Galbeta1-4(Fucalpha1-3)GlcNAcbeta1-3Galbeta1-4(Fucalpha1-3)GlcNAcbeta1-3Galbeta1-4Glcbeta-Cer(d18:1/16:0); Galbeta1-4(Fucalpha1-3)GlcNAcbeta1-3Galbeta1-4(Fucalpha1-3)GlcNAcbeta1-3Galbeta1-4(Fucalpha1-3)GlcNAcbeta1-3Galbeta1-4Glcbeta-Cer(d18:1/18:0); Galbeta1-4(Fucalpha1-3)GlcNAcbeta1-3Galbeta1-4(Fucalpha1-3)GlcNAcbeta1-3Galbeta1-4(Fucalpha1-3)GlcNAcbeta1-3Galbeta1-4Glcbeta-Cer(d18:1/20:0); Galbeta1-4(Fucalpha1-3)GlcNAcbeta1-3Galbeta1-4(Fucalpha1-3)GlcNAcbeta1-3Galbeta1-4(Fucalpha1-3)GlcNAcbeta1-3Galbeta1-4Glcbeta-Cer(d18:1/22:0); Galbeta1-4(Fucalpha1-3)GlcNAcbeta1-3Galbeta1-4(Fucalpha1-3)GlcNAcbeta1-3Galbeta1-4(Fucalpha1-3)GlcNAcbeta1-3Galbeta1-4Glcbeta-Cer(d18:1/24:0); Galbeta1-4(Fucalpha1-3)GlcNAcbeta1-3Galbeta1-4(Fucalpha1-3)GlcNAcbeta1-3Galbeta1-4(Fucalpha1-3)GlcNAcbeta1-3Galbeta1-4Glcbeta-Cer(d18:1/24:1(15Z)); Galbeta1-4(Fucalpha1-3)GlcNAcbeta1-3Galbeta1-4(Fucalpha1-3)GlcNAcbeta1-3Galbeta1-4(Fucalpha1-3)GlcNAcbeta1-3Galbeta1-4Glcbeta-Cer(d18:1/26:0); Galbeta1-4(Fucalpha1-3)GlcNAcbeta1-3Galbeta1-4(Fucalpha1-3)GlcNAcbeta1-3Galbeta1-4(Fucalpha1-3)GlcNAcbeta1-3Galbeta1-4Glcbeta-Cer(d18:1/26:1(17Z)); Galbeta1-4(Fucalpha1-3)GlcNAcbeta1-3Galbeta1-4(Fucalpha1-3)GlcNAcbeta1-3Galbeta1-4Glcbeta-Cer; Galbeta1-4(Fucalpha1-3)GlcNAcbeta1-3Galbeta1-4(Fucalpha1-3)GlcNAcbeta1-3Galbeta1-4Glcbeta-Cer(d18:1/16:0); Galbeta1-4(Fucalpha1-3)GlcNAcbeta1-3Galbeta1-4(Fucalpha1-3)GlcNAcbeta1-3Galbeta1-4Glcbeta-Cer(d18:1/18:0); Galbeta1-4(Fucalpha1-3)GlcNAcbeta1-3Galbeta1-4(Fucalpha1-3)GlcNAcbeta1-3Galbeta1-4Glcbeta-Cer(d18:1/20:0); Galbeta1-4(Fucalpha1-3)GlcNAcbeta1-3Galbeta1-4(Fucalpha1-3)GlcNAcbeta1-3Galbeta1-4Glcbeta-Cer(d18:1/22:0); Galbeta1-4(Fucalpha1-3)GlcNAcbeta1-3Galbeta1-4(Fucalpha1-3)GlcNAcbeta1-3Galbeta1-4Glcbeta-Cer(d18:1/24:0); Galbeta1-4(Fucalpha1-3)GlcNAcbeta1-3Galbeta1-4(Fucalpha1-3)GlcNAcbeta1-3Galbeta1-4Glcbeta-Cer(d18:1/24:1(15Z)); Galbeta1-4(Fucalpha1-3)GlcNAcbeta1-3Galbeta1-4(Fucalpha1-3)GlcNAcbeta1-3Galbeta1-4Glcbeta-Cer(d18:1/26:0); Galbeta1-4(Fucalpha1-3)GlcNAcbeta1-3Galbeta1-4(Fucalpha1-3)GlcNAcbeta1-3Galbeta1-4Glcbeta-Cer(d18:1/26:1(17Z)); Galbeta1-4(Fucalpha1-3)GlcNAcbeta1-3Galbeta1-4(Fucalpha1-3)GlcNAcbeta1-3Galbeta1-4GlcNAcbeta1-3Galbeta1-4Glcbeta-Cer; Galbeta1-4(Fucalpha1-3)GlcNAcbeta1-3Galbeta1-4(Fucalpha1-3)GlcNAcbeta1-3Galbeta1-4GlcNAcbeta1-3Galbeta1-4Glcbeta-Cer(d18:1/16:0); Galbeta1-4(Fucalpha1-3)GlcNAcbeta1-3Galbeta1-4(Fucalpha1-3)GlcNAcbeta1-3Galbeta1-4GlcNAcbeta1-3Galbeta1-4Glcbeta-Cer(d18:1/18:0); Galbeta1-4(Fucalpha1-3)GlcNAcbeta1-3Galbeta1-4(Fucalpha1-3)GlcNAcbeta1-3Galbeta1-4GlcNAcbeta1-3Galbeta1-4Glcbeta-Cer(d18:1/20:0); Galbeta1-4(Fucalpha1-3)GlcNAcbeta1-3Galbeta1-4(Fucalpha1-3)GlcNAcbeta1-3Galbeta1-4GlcNAcbeta1-3Galbeta1-4Glcbeta-Cer(d18:1/22:0); Galbeta1-4(Fucalpha1-3)GlcNAcbeta1-3Galbeta1-4(Fucalpha1-3)GlcNAcbeta1-3Galbeta1-4GlcNAcbeta1-3Galbeta1-4Glcbeta-Cer(d18:1/24:0); Galbeta1-4(Fucalpha1-3)GlcNAcbeta1-3Galbeta1-4(Fucalpha1-3)GlcNAcbeta1-3Galbeta1-4GlcNAcbeta1-3Galbeta1-4Glcbeta-Cer(d18:1/24:1(15Z)); Galbeta1-4(Fucalpha1-3)GlcNAcbeta1-3Galbeta1-4(Fucalpha1-3)GlcNAcbeta1-3Galbeta1-4GlcNAcbeta1-3Galbeta1-4Glcbeta-Cer(d18:1/26:0); Galbeta1-4(Fucalpha1-3)GlcNAcbeta1-3Galbeta1-4(Fucalpha1-3)GlcNAcbeta1-3Galbeta1-4GlcNAcbeta1-3Galbeta1-4Glcbeta-Cer(d18:1/26:1(17Z)); Galbeta1-4(Fucalpha1-3)GlcNAcbeta1-3Galbeta1-4Glcbeta-Cer; Galbeta1-4(Fucalpha1-3)GlcNAcbeta1-3Galbeta1-4Glcbeta-Cer(d18:1/16:0); Galbeta1-4(Fucalpha1-3)GlcNAcbeta1-3Galbeta1-4Glcbeta-Cer(d18:1/18:0); Galbeta1-4(Fucalpha1-3)GlcNAcbeta1-3Galbeta1-4Glcbeta-Cer(d18:1/20:0); Galbeta1-4(Fucalpha1-3)GlcNAcbeta1-3Galbeta1-4Glcbeta-Cer(d18:1/22:0); Galbeta1-4(Fucalpha1-3)GlcNAcbeta1-3Galbeta1-4Glcbeta-Cer(d18:1/24:0); Galbeta1-4(Fucalpha1-3)GlcNAcbeta1-3Galbeta1-4Glcbeta-Cer(d18:1/24:1(15Z)); Galbeta1-4(Fucalpha1-3)GlcNAcbeta1-3Galbeta1-4Glcbeta-Cer(d18:1/26:0); Galbeta1-4(Fucalpha1-3)GlcNAcbeta1-3Galbeta1-4Glcbeta-Cer(d18:1/26:1(17Z)); Galbeta1-4(Fucalpha1-3)GlcNAcbeta1-3Galbeta1-4GlcNAcbeta1-3Galbeta1-4(Fucalpha1-3)GlcNAcbeta1-3Galbeta1-4Glcbeta-Cer; Galbeta1-4(Fucalpha1-3)GlcNAcbeta1-3Galbeta1-4GlcNAcbeta1-3Galbeta1-4(Fucalpha1-3)GlcNAcbeta1-3Galbeta1-4Glcbeta-Cer(d18:1/16:0); Galbeta1-4(Fucalpha1-3)GlcNAcbeta1-3Galbeta1-4GlcNAcbeta1-3Galbeta1-4(Fucalpha1-3)GlcNAcbeta1-3Galbeta1-4Glcbeta-Cer(d18:1/18:0); Galbeta1-4(Fucalpha1-3)GlcNAcbeta1-3Galbeta1-4GlcNAcbeta1-3Galbeta1-4(Fucalpha1-3)GlcNAcbeta1-3Galbeta1-4Glcbeta-Cer(d18:1/20:0); Galbeta1-4(Fucalpha1-3)GlcNAcbeta1-3Galbeta1-4GlcNAcbeta1-3Galbeta1-4(Fucalpha1-3)GlcNAcbeta1-3Galbeta1-4Glcbeta-Cer(d18:1/22:0); Galbeta1-4(Fucalpha1-3)GlcNAcbeta1-3Galbeta1-4GlcNAcbeta1-3Galbeta1-4(Fucalpha1-3)GlcNAcbeta1-3Galbeta1-4Glcbeta-Cer(d18:1/24:0); Galbeta1-4(Fucalpha1-3)GlcNAcbeta1-3Galbeta1-4GlcNAcbeta1-3Galbeta1-4(Fucalpha1-3)GlcNAcbeta1-3Galbeta1-4Glcbeta-Cer(d18:1/24:1(15Z)); Galbeta1-4(Fucalpha1-3)GlcNAcbeta1-3Galbeta1-4GlcNAcbeta1-3Galbeta1-4(Fucalpha1-3)GlcNAcbeta1-3Galbeta1-4Glcbeta-Cer(d18:1/26:0); Galbeta1-4(Fucalpha1-3)GlcNAcbeta1-3Galbeta1-4GlcNAcbeta1-3Galbeta1-4(Fucalpha1-3)GlcNAcbeta1-3Galbeta1-4Glcbeta-Cer(d18:1/26:1(17Z)); Galbeta1-4(Fucalpha1-3)GlcNAcbeta1-3Galbeta1-4GlcNAcbeta1-3Galbeta1-4Glcbeta-Cer; Galbeta1-4(Fucalpha1-3)GlcNAcbeta1-3Galbeta1-4GlcNAcbeta1-3Galbeta1-4Glcbeta-Cer(d18:1/16:0); Galbeta1-4(Fucalpha1-3)GlcNAcbeta1-3Galbeta1-4GlcNAcbeta1-3Galbeta1-4Glcbeta-Cer(d18:1/18:0); Galbeta1-4(Fucalpha1-3)GlcNAcbeta1-3Galbeta1-4GlcNAcbeta1-3Galbeta1-4Glcbeta-Cer(d18:1/20:0); Galbeta1-4(Fucalpha1-3)GlcNAcbeta1-3Galbeta1-4GlcNAcbeta1-3Galbeta1-4Glcbeta-Cer(d18:1/22:0); Galbeta1-4(Fucalpha1-3)GlcNAcbeta1-3Galbeta1-4GlcNAcbeta1-3Galbeta1-4Glcbeta-Cer(d18:1/24:0); Galbeta1-4(Fucalpha1-3)GlcNAcbeta1-3Galbeta1-4GlcNAcbeta1-3Galbeta1-4Glcbeta-Cer(d18:1/24:1(15Z)); Galbeta1-4(Fucalpha1-3)GlcNAcbeta1-3Galbeta1-4GlcNAcbeta1-3Galbeta1-4Glcbeta-Cer(d18:1/26:0); Galbeta1-4(Fucalpha1-3)GlcNAcbeta1-3Galbeta1-4GlcNAcbeta1-3Galbeta1-4Glcbeta-Cer(d18:1/26:1(17Z)); Galbeta1-4(Fucalpha1-3)GlcNAcbeta1-3Galbeta1-4GlcNAcbeta1-3Galbeta1-4GlcNAcbeta1-3Galbeta1-4Glcbeta-Cer; Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/16:0); Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/18:0); Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/20:0); Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/22:0); Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/24:0); Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/24:1(15Z)); Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/26:0); Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/26:1(17Z)); Galbeta1-4(Fucalpha1-3)GlcNAcbeta1-6(Galbeta1-3)GalNAcbeta1-3Galalpha1-4Galbeta1-4Glcbeta-Cer; Galbeta1-4(Fucalpha1-3)GlcNAcbeta1-6(Galbeta1-3)GalNAcbeta1-3Galalpha1-4Galbeta1-4Glcbeta-Cer(d18:1/16:0); Galbeta1-4(Fucalpha1-3)GlcNAcbeta1-6(Galbeta1-3)GalNAcbeta1-3Galalpha1-4Galbeta1-4Glcbeta-Cer(d18:1/18:0); Galbeta1-4(Fucalpha1-3)GlcNAcbeta1-6(Galbeta1-3)GalNAcbeta1-3Galalpha1-4Galbeta1-4Glcbeta-Cer(d18:1/20:0); Galbeta1-4(Fucalpha1-3)GlcNAcbeta1-6(Galbeta1-3)GalNAcbeta1-3Galalpha1-4Galbeta1-4Glcbeta-Cer(d18:1/22:0); Galbeta1-4(Fucalpha1-3)GlcNAcbeta1-6(Galbeta1-3)GalNAcbeta1-3Galalpha1-4Galbeta1-4Glcbeta-Cer(d18:1/24:0); Galbeta1-4(Fucalpha1-3)GlcNAcbeta1-6(Galbeta1-3)GalNAcbeta1-3Galalpha1-4Galbeta1-4Glcbeta-Cer(d18:1/24:1(15Z)); Galbeta1-4(Fucalpha1-3)GlcNAcbeta1-6(Galbeta1-3)GalNAcbeta1-3Galalpha1-4Galbeta1-4Glcbeta-Cer(d18:1/26:0); Galbeta1-4(Fucalpha1-3)GlcNAcbeta1-6(Galbeta1-3)GalNAcbeta1-3Galalpha1-4Galbeta1-4Glcbeta-Cer(d18:1/26:1(17Z)); Galbeta1-4(NeuAcalpha2-3)Galbeta1-4Glcbeta-Cer; Galbeta1-4(NeuAcalpha2-3)Galbeta1-4Glcbeta-Cer(d18:1/16:0); Galbeta1-4(NeuAcalpha2-3)Galbeta1-4Glcbeta-Cer(d18:1/18:0); Galbeta1-4(NeuAcalpha2-3)Galbeta1-4Glcbeta-Cer(d18:1/20:0); Galbeta1-4(NeuAcalpha2-3)Galbeta1-4Glcbeta-Cer(d18:1/22:0); Galbeta1-4(NeuAcalpha2-3)Galbeta1-4Glcbeta-Cer(d18:1/24:0); Galbeta1-4(NeuAcalpha2-3)Galbeta1-4Glcbeta-Cer(d18:1/24:1(15Z)); Galbeta1-4(NeuAcalpha2-3)Galbeta1-4Glcbeta-Cer(d18:1/26:0); Galbeta1-4(NeuAcalpha2-3)Galbeta1-4Glcbeta-Cer(d18:1/26:1(17Z)); Galbeta1-4Galbeta1-4GlcNAcbeta1-3Galbeta1-4Glcbeta-Cer; Galbeta1-4GalNAcbeta1-3Galbeta1-3GalNAcbeta1-4Galbeta1-4Glcbeta-Cer; Galbeta1-4GalNAcbeta1-3Galbeta1-3GalNAcbeta1-4Galbeta1-4Glcbeta-Cer(d18:1/16:0); Galbeta1-4GalNAcbeta1-3Galbeta1-3GalNAcbeta1-4Galbeta1-4Glcbeta-Cer(d18:1/18:0); Galbeta1-4GalNAcbeta1-3Galbeta1-3GalNAcbeta1-4Galbeta1-4Glcbeta-Cer(d18:1/20:0); Galbeta1-4GalNAcbeta1-3Galbeta1-3GalNAcbeta1-4Galbeta1-4Glcbeta-Cer(d18:1/22:0); Galbeta1-4GalNAcbeta1-3Galbeta1-3GalNAcbeta1-4Galbeta1-4Glcbeta-Cer(d18:1/24:0); Galbeta1-4GalNAcbeta1-3Galbeta1-3GalNAcbeta1-4Galbeta1-4Glcbeta-Cer(d18:1/24:1(15Z)); Galbeta1-4GalNAcbeta1-3Galbeta1-3GalNAcbeta1-4Galbeta1-4Glcbeta-Cer(d18:1/26:0); Galbeta1-4GalNAcbeta1-3Galbeta1-3GalNAcbeta1-4Galbeta1-4Glcbeta-Cer(d18:1/26:1(17Z)); Galbeta1-4Glcbeta-Cer; Galbeta1-4GlcNAcalpha1-3Galbeta1-4GlcNAcbeta1-3Galbeta1-4Glcbeta-Cer; Galbeta1-4GlcNAcalpha1-3Galbeta1-4GlcNAcbeta1-3Galbeta1-4Glcbeta-Cer(d18:1/16:0); Galbeta1-4GlcNAcalpha1-3Galbeta1-4GlcNAcbeta1-3Galbeta1-4Glcbeta-Cer(d18:1/18:0); Galbeta1-4GlcNAcalpha1-3Galbeta1-4GlcNAcbeta1-3Galbeta1-4Glcbeta-Cer(d18:1/20:0); Galbeta1-4GlcNAcalpha1-3Galbeta1-4GlcNAcbeta1-3Galbeta1-4Glcbeta-Cer(d18:1/22:0); Galbeta1-4GlcNAcalpha1-3Galbeta1-4GlcNAcbeta1-3Galbeta1-4Glcbeta-Cer(d18:1/24:0); Galbeta1-4GlcNAcalpha1-3Galbeta1-4GlcNAcbeta1-3Galbeta1-4Glcbeta-Cer(d18:1/24:1(15Z)); Galbeta1-4GlcNAcalpha1-3Galbeta1-4GlcNAcbeta1-3Galbeta1-4Glcbeta-Cer(d18:1/26:0); Galbeta1-4GlcNAcalpha1-3Galbeta1-4GlcNAcbeta1-3Galbeta1-4Glcbeta-Cer(d18:1/26:1(17Z)); Galbeta1-4GlcNAcbeta1-3(Fucalpha1-2Galbeta1-4GlcNAcbeta1-6)Galbeta1-4GlcNAcbeta1-3Galbeta1-4Glcbeta-Cer; Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/16:0); Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/18:0); Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/20:0); Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/22:0); Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/24:0); Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/24:1(15Z)); Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/26:0); Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/26:1(17Z)); Galbeta1-4GlcNAcbeta1-3(Galalpha1-3Galbeta1-4GlcNAcbeta1-6)Galbeta1-4GlcNAcbeta1-3Galbeta1-4Glcbeta-Cer; Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/16:0); Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/18:0); Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/20:0); Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/22:0); Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:0); Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:1(15Z)); Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:0); Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:1(17Z)); Galbeta1-4GlcNAcbeta1-3(Galbeta1-4GlcNAcbeta1-6)Galbeta1-4Glcbeta-Cer; Galbeta1-4GlcNAcbeta1-3(Galbeta1-4GlcNAcbeta1-6)Galbeta1-4Glcbeta-Cer(d18:1/16:0); Galbeta1-4GlcNAcbeta1-3(Galbeta1-4GlcNAcbeta1-6)Galbeta1-4Glcbeta-Cer(d18:1/18:0); Galbeta1-4GlcNAcbeta1-3(Galbeta1-4GlcNAcbeta1-6)Galbeta1-4Glcbeta-Cer(d18:1/20:0); Galbeta1-4GlcNAcbeta1-3(Galbeta1-4GlcNAcbeta1-6)Galbeta1-4Glcbeta-Cer(d18:1/22:0); Galbeta1-4GlcNAcbeta1-3(Galbeta1-4GlcNAcbeta1-6)Galbeta1-4Glcbeta-Cer(d18:1/24:0); Galbeta1-4GlcNAcbeta1-3(Galbeta1-4GlcNAcbeta1-6)Galbeta1-4Glcbeta-Cer(d18:1/24:1(15Z)); Galbeta1-4GlcNAcbeta1-3(Galbeta1-4GlcNAcbeta1-6)Galbeta1-4Glcbeta-Cer(d18:1/26:0); Galbeta1-4GlcNAcbeta1-3(Galbeta1-4GlcNAcbeta1-6)Galbeta1-4Glcbeta-Cer(d18:1/26:1(17Z)); Galbeta1-4GlcNAcbeta1-3(Galbeta1-4GlcNAcbeta1-6)Galbeta1-4GlcNAcbeta1-3Galbeta1-4Glcbeta-Cer; Galbeta1-4GlcNAcbeta1-3(Galbeta1-4GlcNAcbeta1-6)Galbeta1-4GlcNAcbeta1-3Galbeta1-4Glcbeta-Cer(d18:1/16:0); Galbeta1-4GlcNAcbeta1-3(Galbeta1-4GlcNAcbeta1-6)Galbeta1-4GlcNAcbeta1-3Galbeta1-4Glcbeta-Cer(d18:1/18:0); Galbeta1-4GlcNAcbeta1-3(Galbeta1-4GlcNAcbeta1-6)Galbeta1-4GlcNAcbeta1-3Galbeta1-4Glcbeta-Cer(d18:1/20:0); Galbeta1-4GlcNAcbeta1-3(Galbeta1-4GlcNAcbeta1-6)Galbeta1-4GlcNAcbeta1-3Galbeta1-4Glcbeta-Cer(d18:1/22:0); Galbeta1-4GlcNAcbeta1-3(Galbeta1-4GlcNAcbeta1-6)Galbeta1-4GlcNAcbeta1-3Galbeta1-4Glcbeta-Cer(d18:1/24:0); Galbeta1-4GlcNAcbeta1-3(Galbeta1-4GlcNAcbeta1-6)Galbeta1-4GlcNAcbeta1-3Galbeta1-4Glcbeta-Cer(d18:1/24:1(15Z)); Galbeta1-4GlcNAcbeta1-3(Galbeta1-4GlcNAcbeta1-6)Galbeta1-4GlcNAcbeta1-3Galbeta1-4Glcbeta-Cer(d18:1/26:0); Galbeta1-4GlcNAcbeta1-3(Galbeta1-4GlcNAcbeta1-6)Galbeta1-4GlcNAcbeta1-3Galbeta1-4Glcbeta-Cer(d18:1/26:1(17Z)); Galbeta1-4GlcNAcbeta1-3(GlcNAcbeta1-6)Galbeta1-4GlcNAcbeta1-3Galbeta1-4Glcbeta-Cer; Galbeta1-4GlcNAcbeta1-3(GlcNAcbeta1-6)Galbeta1-4GlcNAcbeta1-3Galbeta1-4Glcbeta-Cer(d18:1/16:0); Galbeta1-4GlcNAcbeta1-3(GlcNAcbeta1-6)Galbeta1-4GlcNAcbeta1-3Galbeta1-4Glcbeta-Cer(d18:1/18:0); Galbeta1-4GlcNAcbeta1-3(GlcNAcbeta1-6)Galbeta1-4GlcNAcbeta1-3Galbeta1-4Glcbeta-Cer(d18:1/20:0); Galbeta1-4GlcNAcbeta1-3(GlcNAcbeta1-6)Galbeta1-4GlcNAcbeta1-3Galbeta1-4Glcbeta-Cer(d18:1/22:0); Galbeta1-4GlcNAcbeta1-3(GlcNAcbeta1-6)Galbeta1-4GlcNAcbeta1-3Galbeta1-4Glcbeta-Cer(d18:1/24:0); Galbeta1-4GlcNAcbeta1-3(GlcNAcbeta1-6)Galbeta1-4GlcNAcbeta1-3Galbeta1-4Glcbeta-Cer(d18:1/24:1(15Z)); Galbeta1-4GlcNAcbeta1-3(GlcNAcbeta1-6)Galbeta1-4GlcNAcbeta1-3Galbeta1-4Glcbeta-Cer(d18:1/26:0); Galbeta1-4GlcNAcbeta1-3(GlcNAcbeta1-6)Galbeta1-4GlcNAcbeta1-3Galbeta1-4Glcbeta-Cer(d18:1/26:1(17Z)); Galbeta1-4GlcNAcbeta1-3Galalpha1-3(GalNAcbeta1-4)Galbeta1-4Glcbeta-Cer; Galbeta1-4GlcNAcbeta1-3Galalpha1-3(GalNAcbeta1-4)Galbeta1-4Glcbeta-Cer(d18:1/16:0); Galbeta1-4GlcNAcbeta1-3Galalpha1-3(GalNAcbeta1-4)Galbeta1-4Glcbeta-Cer(d18:1/18:0); Galbeta1-4GlcNAcbeta1-3Galalpha1-3(GalNAcbeta1-4)Galbeta1-4Glcbeta-Cer(d18:1/20:0); Galbeta1-4GlcNAcbeta1-3Galalpha1-3(GalNAcbeta1-4)Galbeta1-4Glcbeta-Cer(d18:1/22:0); Galbeta1-4GlcNAcbeta1-3Galalpha1-3(GalNAcbeta1-4)Galbeta1-4Glcbeta-Cer(d18:1/24:0); Galbeta1-4GlcNAcbeta1-3Galalpha1-3(GalNAcbeta1-4)Galbeta1-4Glcbeta-Cer(d18:1/24:1(15Z)); Galbeta1-4GlcNAcbeta1-3Galalpha1-3(GalNAcbeta1-4)Galbeta1-4Glcbeta-Cer(d18:1/26:0); Galbeta1-4GlcNAcbeta1-3Galalpha1-3(GalNAcbeta1-4)Galbeta1-4Glcbeta-Cer(d18:1/26:1(17Z)); Galbeta1-4GlcNAcbeta1-3Galalpha1-3Galbeta1-4Glcbeta-Cer; Galbeta1-4GlcNAcbeta1-3Galalpha1-3Galbeta1-4Glcbeta-Cer(d18:1/16:0); Galbeta1-4GlcNAcbeta1-3Galalpha1-3Galbeta1-4Glcbeta-Cer(d18:1/18:0); Galbeta1-4GlcNAcbeta1-3Galalpha1-3Galbeta1-4Glcbeta-Cer(d18:1/20:0); Galbeta1-4GlcNAcbeta1-3Galalpha1-3Galbeta1-4Glcbeta-Cer(d18:1/22:0); Galbeta1-4GlcNAcbeta1-3Galalpha1-3Galbeta1-4Glcbeta-Cer(d18:1/24:0); Galbeta1-4GlcNAcbeta1-3Galalpha1-3Galbeta1-4Glcbeta-Cer(d18:1/24:1(15Z)); Galbeta1-4GlcNAcbeta1-3Galalpha1-3Galbeta1-4Glcbeta-Cer(d18:1/26:0); Galbeta1-4GlcNAcbeta1-3Galalpha1-3Galbeta1-4Glcbeta-Cer(d18:1/26:1(17Z)); Galbeta1-4GlcNAcbeta1-3Galbeta1-3(Fucalpha1-4)GlcNAcbeta1-3Galbeta1-4Glcbeta-Cer; Galbeta1-4GlcNAcbeta1-3Galbeta1-3(Fucalpha1-4)GlcNAcbeta1-3Galbeta1-4Glcbeta-Cer(d18:1/16:0); Galbeta1-4GlcNAcbeta1-3Galbeta1-3(Fucalpha1-4)GlcNAcbeta1-3Galbeta1-4Glcbeta-Cer(d18:1/18:0); Galbeta1-4GlcNAcbeta1-3Galbeta1-3(Fucalpha1-4)GlcNAcbeta1-3Galbeta1-4Glcbeta-Cer(d18:1/20:0); Galbeta1-4GlcNAcbeta1-3Galbeta1-3(Fucalpha1-4)GlcNAcbeta1-3Galbeta1-4Glcbeta-Cer(d18:1/22:0); Galbeta1-4GlcNAcbeta1-3Galbeta1-3(Fucalpha1-4)GlcNAcbeta1-3Galbeta1-4Glcbeta-Cer(d18:1/24:0); Galbeta1-4GlcNAcbeta1-3Galbeta1-3(Fucalpha1-4)GlcNAcbeta1-3Galbeta1-4Glcbeta-Cer(d18:1/24:1(15Z)); Galbeta1-4GlcNAcbeta1-3Galbeta1-3(Fucalpha1-4)GlcNAcbeta1-3Galbeta1-4Glcbeta-Cer(d18:1/26:0); Galbeta1-4GlcNAcbeta1-3Galbeta1-3(Fucalpha1-4)GlcNAcbeta1-3Galbeta1-4Glcbeta-Cer(d18:1/26:1(17Z)); Galbeta1-4GlcNAcbeta1-3Galbeta1-3GalNAcbeta1-4(NeuAcalpha2-3)Galbeta1-4Glcbeta-Cer; Galbeta1-4GlcNAcbeta1-3Galbeta1-3GalNAcbeta1-4(NeuAcalpha2-3)Galbeta1-4Glcbeta-Cer(d18:1/16:0); Galbeta1-4GlcNAcbeta1-3Galbeta1-3GalNAcbeta1-4(NeuAcalpha2-3)Galbeta1-4Glcbeta-Cer(d18:1/18:0); Galbeta1-4GlcNAcbeta1-3Galbeta1-3GalNAcbeta1-4(NeuAcalpha2-3)Galbeta1-4Glcbeta-Cer(d18:1/20:0); Galbeta1-4GlcNAcbeta1-3Galbeta1-3GalNAcbeta1-4(NeuAcalpha2-3)Galbeta1-4Glcbeta-Cer(d18:1/22:0); Galbeta1-4GlcNAcbeta1-3Galbeta1-3GalNAcbeta1-4(NeuAcalpha2-3)Galbeta1-4Glcbeta-Cer(d18:1/24:0); Galbeta1-4GlcNAcbeta1-3Galbeta1-3GalNAcbeta1-4(NeuAcalpha2-3)Galbeta1-4Glcbeta-Cer(d18:1/24:1(15Z)); Galbeta1-4GlcNAcbeta1-3Galbeta1-3GalNAcbeta1-4(NeuAcalpha2-3)Galbeta1-4Glcbeta-Cer(d18:1/26:0); Galbeta1-4GlcNAcbeta1-3Galbeta1-3GalNAcbeta1-4(NeuAcalpha2-3)Galbeta1-4Glcbeta-Cer(d18:1/26:1(17Z)); Galbeta1-4GlcNAcbeta1-3Galbeta1-3GalNAcbeta1-4Galbeta1-4Glcbeta-Cer; Galbeta1-4GlcNAcbeta1-3Galbeta1-3GalNAcbeta1-4Galbeta1-4Glcbeta-Cer(d18:1/16:0); Galbeta1-4GlcNAcbeta1-3Galbeta1-3GalNAcbeta1-4Galbeta1-4Glcbeta-Cer(d18:1/18:0); Galbeta1-4GlcNAcbeta1-3Galbeta1-3GalNAcbeta1-4Galbeta1-4Glcbeta-Cer(d18:1/20:0); Galbeta1-4GlcNAcbeta1-3Galbeta1-3GalNAcbeta1-4Galbeta1-4Glcbeta-Cer(d18:1/22:0); Galbeta1-4GlcNAcbeta1-3Galbeta1-3GalNAcbeta1-4Galbeta1-4Glcbeta-Cer(d18:1/24:0); Galbeta1-4GlcNAcbeta1-3Galbeta1-3GalNAcbeta1-4Galbeta1-4Glcbeta-Cer(d18:1/24:1(15Z)); Galbeta1-4GlcNAcbeta1-3Galbeta1-3GalNAcbeta1-4Galbeta1-4Glcbeta-Cer(d18:1/26:0); Galbeta1-4GlcNAcbeta1-3Galbeta1-3GalNAcbeta1-4Galbeta1-4Glcbeta-Cer(d18:1/26:1(17Z)); Galbeta1-4GlcNAcbeta1-3Galbeta1-3GlcNAcbeta1-3Galbeta1-4Glcbeta-Cer; Galbeta1-4GlcNAcbeta1-3Galbeta1-3GlcNAcbeta1-3Galbeta1-4Glcbeta-Cer(d18:1/16:0); Galbeta1-4GlcNAcbeta1-3Galbeta1-3GlcNAcbeta1-3Galbeta1-4Glcbeta-Cer(d18:1/18:0); Galbeta1-4GlcNAcbeta1-3Galbeta1-3GlcNAcbeta1-3Galbeta1-4Glcbeta-Cer(d18:1/20:0); Galbeta1-4GlcNAcbeta1-3Galbeta1-3GlcNAcbeta1-3Galbeta1-4Glcbeta-Cer(d18:1/22:0); Galbeta1-4GlcNAcbeta1-3Galbeta1-3GlcNAcbeta1-3Galbeta1-4Glcbeta-Cer(d18:1/24:0); Galbeta1-4GlcNAcbeta1-3Galbeta1-3GlcNAcbeta1-3Galbeta1-4Glcbeta-Cer(d18:1/24:1(15Z)); Galbeta1-4GlcNAcbeta1-3Galbeta1-3GlcNAcbeta1-3Galbeta1-4Glcbeta-Cer(d18:1/26:0); Galbeta1-4GlcNAcbeta1-3Galbeta1-3GlcNAcbeta1-3Galbeta1-4Glcbeta-Cer(d18:1/26:1(17Z)); Galbeta1-4GlcNAcbeta1-3Galbeta1-4(Fucalpha1-3)GlcNAcbeta1-3Galbeta1-4Glcbeta-Cer; Galbeta1-4GlcNAcbeta1-3Galbeta1-4(Fucalpha1-3)GlcNAcbeta1-3Galbeta1-4Glcbeta-Cer(d18:1/16:0); Galbeta1-4GlcNAcbeta1-3Galbeta1-4(Fucalpha1-3)GlcNAcbeta1-3Galbeta1-4Glcbeta-Cer(d18:1/18:0); Galbeta1-4GlcNAcbeta1-3Galbeta1-4(Fucalpha1-3)GlcNAcbeta1-3Galbeta1-4Glcbeta-Cer(d18:1/20:0); Galbeta1-4GlcNAcbeta1-3Galbeta1-4(Fucalpha1-3)GlcNAcbeta1-3Galbeta1-4Glcbeta-Cer(d18:1/22:0); Galbeta1-4GlcNAcbeta1-3Galbeta1-4(Fucalpha1-3)GlcNAcbeta1-3Galbeta1-4Glcbeta-Cer(d18:1/24:0); Galbeta1-4GlcNAcbeta1-3Galbeta1-4(Fucalpha1-3)GlcNAcbeta1-3Galbeta1-4Glcbeta-Cer(d18:1/24:1(15Z)); Galbeta1-4GlcNAcbeta1-3Galbeta1-4(Fucalpha1-3)GlcNAcbeta1-3Galbeta1-4Glcbeta-Cer(d18:1/26:0); Galbeta1-4GlcNAcbeta1-3Galbeta1-4(Fucalpha1-3)GlcNAcbeta1-3Galbeta1-4Glcbeta-Cer(d18:1/26:1(17Z)); Galbeta1-4GlcNAcbeta1-3Galbeta1-4(Fucalpha1-3)GlcNAcbeta1-3Galbeta1-4GlcNAcbeta1-3Galbeta1-4Glcbeta-Cer; Galbeta1-4GlcNAcbeta1-3Galbeta1-4(Fucalpha1-3)GlcNAcbeta1-3Galbeta1-4GlcNAcbeta1-3Galbeta1-4Glcbeta-Cer(d18:1/16:0); Galbeta1-4GlcNAcbeta1-3Galbeta1-4(Fucalpha1-3)GlcNAcbeta1-3Galbeta1-4GlcNAcbeta1-3Galbeta1-4Glcbeta-Cer(d18:1/18:0); Galbeta1-4GlcNAcbeta1-3Galbeta1-4(Fucalpha1-3)GlcNAcbeta1-3Galbeta1-4GlcNAcbeta1-3Galbeta1-4Glcbeta-Cer(d18:1/20:0); Galbeta1-4GlcNAcbeta1-3Galbeta1-4(Fucalpha1-3)GlcNAcbeta1-3Galbeta1-4GlcNAcbeta1-3Galbeta1-4Glcbeta-Cer(d18:1/22:0); Galbeta1-4GlcNAcbeta1-3Galbeta1-4(Fucalpha1-3)GlcNAcbeta1-3Galbeta1-4GlcNAcbeta1-3Galbeta1-4Glcbeta-Cer(d18:1/24:0); Galbeta1-4GlcNAcbeta1-3Galbeta1-4(Fucalpha1-3)GlcNAcbeta1-3Galbeta1-4GlcNAcbeta1-3Galbeta1-4Glcbeta-Cer(d18:1/24:1(15Z)); Galbeta1-4GlcNAcbeta1-3Galbeta1-4(Fucalpha1-3)GlcNAcbeta1-3Galbeta1-4GlcNAcbeta1-3Galbeta1-4Glcbeta-Cer(d18:1/26:0); Galbeta1-4GlcNAcbeta1-3Galbeta1-4(Fucalpha1-3)GlcNAcbeta1-3Galbeta1-4GlcNAcbeta1-3Galbeta1-4Glcbeta-Cer(d18:1/26:1(17Z)); Galbeta1-4GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer; Galbeta1-4GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/16:0); Galbeta1-4GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/18:0); Galbeta1-4GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/20:0); Galbeta1-4GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/22:0); Galbeta1-4GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/24:0); Galbeta1-4GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/24:1(15Z)); Galbeta1-4GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/26:0); Galbeta1-4GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/26:1(17Z)); Galbeta1-4GlcNAcbeta1-3Galbeta1-4Glcbeta-Cer; Galbeta1-4GlcNAcbeta1-3Galbeta1-4Glcbeta-Cer(d18:1/16:0); Galbeta1-4GlcNAcbeta1-3Galbeta1-4Glcbeta-Cer(d18:1/18:0); Galbeta1-4GlcNAcbeta1-3Galbeta1-4Glcbeta-Cer(d18:1/20:0); Galbeta1-4GlcNAcbeta1-3Galbeta1-4Glcbeta-Cer(d18:1/22:0); Galbeta1-4GlcNAcbeta1-3Galbeta1-4Glcbeta-Cer(d18:1/24:0); Galbeta1-4GlcNAcbeta1-3Galbeta1-4Glcbeta-Cer(d18:1/24:1(15Z)); Galbeta1-4GlcNAcbeta1-3Galbeta1-4Glcbeta-Cer(d18:1/26:0); Galbeta1-4GlcNAcbeta1-3Galbeta1-4Glcbeta-Cer(d18:1/26:1(17Z)); Galbeta1-4GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer; Galbeta1-4GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/16:0); Galbeta1-4GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/18:0); Galbeta1-4GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/20:0); Galbeta1-4GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/22:0); Galbeta1-4GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:0); Galbeta1-4GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:1(15Z)); Galbeta1-4GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:0); Galbeta1-4GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:1(17Z)); Galbeta1-4GlcNAcbeta1-3Galbeta1-4GlcNAcbeta1-3Galbeta1-4Glcbeta-Cer; Galbeta1-4GlcNAcbeta1-3Galbeta1-4GlcNAcbeta1-3Galbeta1-4Glcbeta-Cer(d18:1/16:0); Galbeta1-4GlcNAcbeta1-3Galbeta1-4GlcNAcbeta1-3Galbeta1-4Glcbeta-Cer(d18:1/18:0); Galbeta1-4GlcNAcbeta1-3Galbeta1-4GlcNAcbeta1-3Galbeta1-4Glcbeta-Cer(d18:1/20:0); Galbeta1-4GlcNAcbeta1-3Galbeta1-4GlcNAcbeta1-3Galbeta1-4Glcbeta-Cer(d18:1/22:0); Galbeta1-4GlcNAcbeta1-3Galbeta1-4GlcNAcbeta1-3Galbeta1-4Glcbeta-Cer(d18:1/24:0); Galbeta1-4GlcNAcbeta1-3Galbeta1-4GlcNAcbeta1-3Galbeta1-4Glcbeta-Cer(d18:1/24:1(15Z)); Galbeta1-4GlcNAcbeta1-3Galbeta1-4GlcNAcbeta1-3Galbeta1-4Glcbeta-Cer(d18:1/26:0); Galbeta1-4GlcNAcbeta1-3Galbeta1-4GlcNAcbeta1-3Galbeta1-4Glcbeta-Cer(d18:1/26:1(17Z)); Galbeta1-4GlcNAcbeta1-3Galbeta1-4GlcNAcbeta1-3Galbeta1-4GlcNAcbeta1-3Galbeta1-4Glcbeta-Cer; Galbeta1-4GlcNAcbeta1-3Galbeta1-4GlcNAcbeta1-3Galbeta1-4GlcNAcbeta1-3Galbeta1-4Glcbeta-Cer(d18:1/16:0); Galbeta1-4GlcNAcbeta1-3Galbeta1-4GlcNAcbeta1-3Galbeta1-4GlcNAcbeta1-3Galbeta1-4Glcbeta-Cer(d18:1/18:0); Galbeta1-4GlcNAcbeta1-3Galbeta1-4GlcNAcbeta1-3Galbeta1-4GlcNAcbeta1-3Galbeta1-4Glcbeta-Cer(d18:1/20:0); Galbeta1-4GlcNAcbeta1-3Galbeta1-4GlcNAcbeta1-3Galbeta1-4GlcNAcbeta1-3Galbeta1-4Glcbeta-Cer(d18:1/22:0); Galbeta1-4GlcNAcbeta1-3Galbeta1-4GlcNAcbeta1-3Galbeta1-4GlcNAcbeta1-3Galbeta1-4Glcbeta-Cer(d18:1/24:0); Galbeta1-4GlcNAcbeta1-3Galbeta1-4GlcNAcbeta1-3Galbeta1-4GlcNAcbeta1-3Galbeta1-4Glcbeta-Cer(d18:1/24:1(15Z)); Galbeta1-4GlcNAcbeta1-3Galbeta1-4GlcNAcbeta1-3Galbeta1-4GlcNAcbeta1-3Galbeta1-4Glcbeta-Cer(d18:1/26:0); Galbeta1-4GlcNAcbeta1-3Galbeta1-4GlcNAcbeta1-3Galbeta1-4GlcNAcbeta1-3Galbeta1-4Glcbeta-Cer(d18:1/26:1(17Z)); Galbeta1-4GlcNAcbeta1-3Galbeta1-4GlcNAcbeta1-3Galbeta1-4GlcNAcbeta1-3Galbeta1-4GlcNAcbeta1-3Galbeta1-4Glcbeta-Cer; Galbeta1-4GlcNAcbeta1-3Galbeta1-4GlcNAcbeta1-3Galbeta1-4GlcNAcbeta1-3Galbeta1-4GlcNAcbeta1-3Galbeta1-4Glcbeta-Cer(d18:1/16:0); Galbeta1-4GlcNAcbeta1-3Galbeta1-4GlcNAcbeta1-3Galbeta1-4GlcNAcbeta1-3Galbeta1-4GlcNAcbeta1-3Galbeta1-4Glcbeta-Cer(d18:1/18:0); Galbeta1-4GlcNAcbeta1-3Galbeta1-4GlcNAcbeta1-3Galbeta1-4GlcNAcbeta1-3Galbeta1-4GlcNAcbeta1-3Galbeta1-4Glcbeta-Cer(d18:1/20:0); Galbeta1-4GlcNAcbeta1-3Galbeta1-4GlcNAcbeta1-3Galbeta1-4GlcNAcbeta1-3Galbeta1-4GlcNAcbeta1-3Galbeta1-4Glcbeta-Cer(d18:1/22:0); Galbeta1-4GlcNAcbeta1-3Galbeta1-4GlcNAcbeta1-3Galbeta1-4GlcNAcbeta1-3Galbeta1-4GlcNAcbeta1-3Galbeta1-4Glcbeta-Cer(d18:1/24:0); Galbeta1-4GlcNAcbeta1-3Galbeta1-4GlcNAcbeta1-3Galbeta1-4GlcNAcbeta1-3Galbeta1-4GlcNAcbeta1-3Galbeta1-4Glcbeta-Cer(d18:1/24:1(15Z)); Galbeta1-4GlcNAcbeta1-3Galbeta1-4GlcNAcbeta1-3Galbeta1-4GlcNAcbeta1-3Galbeta1-4GlcNAcbeta1-3Galbeta1-4Glcbeta-Cer(d18:1/26:0); Galbeta1-4GlcNAcbeta1-3Galbeta1-4GlcNAcbeta1-3Galbeta1-4GlcNAcbeta1-3Galbeta1-4GlcNAcbeta1-3Galbeta1-4Glcbeta-Cer(d18:1/26:1(17Z)); Galbeta1-4GlcNAcbeta1-6GalNAcbeta1-3Galalpha1-4Galbeta1-4Glcbeta-Cer; Galbeta1-4GlcNAcbeta1-6GalNAcbeta1-3Galalpha1-4Galbeta1-4Glcbeta-Cer(d18:1/16:0); Galbeta1-4GlcNAcbeta1-6GalNAcbeta1-3Galalpha1-4Galbeta1-4Glcbeta-Cer(d18:1/18:0); Galbeta1-4GlcNAcbeta1-6GalNAcbeta1-3Galalpha1-4Galbeta1-4Glcbeta-Cer(d18:1/20:0); Galbeta1-4GlcNAcbeta1-6GalNAcbeta1-3Galalpha1-4Galbeta1-4Glcbeta-Cer(d18:1/22:0); Galbeta1-4GlcNAcbeta1-6GalNAcbeta1-3Galalpha1-4Galbeta1-4Glcbeta-Cer(d18:1/24:0); Galbeta1-4GlcNAcbeta1-6GalNAcbeta1-3Galalpha1-4Galbeta1-4Glcbeta-Cer(d18:1/24:1(15Z)); Galbeta1-4GlcNAcbeta1-6GalNAcbeta1-3Galalpha1-4Galbeta1-4Glcbeta-Cer(d18:1/26:0); Galbeta1-4GlcNAcbeta1-6GalNAcbeta1-3Galalpha1-4Galbeta1-4Glcbeta-Cer(d18:1/26:1(17Z)); Galbeta1-4HSO3-6GlcNAcbeta1-3Galbeta1-4Glcbeta-Cer; Galbeta-Cer; Galbeta-Cer(d18:1/16:0); Galbeta-Cer(d18:1/18:0); Galbeta-Cer(d18:1/20:0); Galbeta-Cer(d18:1/22:0); Galbeta-Cer(d18:1/24:0); Galbeta-Cer(d18:1/24:1(15Z)); Galbeta-Cer(d18:1/26:0); Galbeta-Cer(d18:1/26:1(17Z)); GalNAcalpha1-3(Fucalpha1-2)Galbeta1-3(Fucalpha1-4)GlcNAcbeta1-3Galbeta1-4Glcbeta-Cer; GalNAcalpha1-3(Fucalpha1-2)Galbeta1-3(Fucalpha1-4)GlcNAcbeta1-3Galbeta1-4Glcbeta-Cer(d18:1/16:0); GalNAcalpha1-3(Fucalpha1-2)Galbeta1-3(Fucalpha1-4)GlcNAcbeta1-3Galbeta1-4Glcbeta-Cer(d18:1/18:0); GalNAcalpha1-3(Fucalpha1-2)Galbeta1-3(Fucalpha1-4)GlcNAcbeta1-3Galbeta1-4Glcbeta-Cer(d18:1/20:0); GalNAcalpha1-3(Fucalpha1-2)Galbeta1-3(Fucalpha1-4)GlcNAcbeta1-3Galbeta1-4Glcbeta-Cer(d18:1/22:0); GalNAcalpha1-3(Fucalpha1-2)Galbeta1-3(Fucalpha1-4)GlcNAcbeta1-3Galbeta1-4Glcbeta-Cer(d18:1/24:0); GalNAcalpha1-3(Fucalpha1-2)Galbeta1-3(Fucalpha1-4)GlcNAcbeta1-3Galbeta1-4Glcbeta-Cer(d18:1/24:1(15Z)); GalNAcalpha1-3(Fucalpha1-2)Galbeta1-3(Fucalpha1-4)GlcNAcbeta1-3Galbeta1-4Glcbeta-Cer(d18:1/26:0); GalNAcalpha1-3(Fucalpha1-2)Galbeta1-3(Fucalpha1-4)GlcNAcbeta1-3Galbeta1-4Glcbeta-Cer(d18:1/26:1(17Z)); GalNAcalpha1-3(Fucalpha1-2)Galbeta1-3GalNAcalpha1-3(Fucalpha1-2)Galbeta1-4GlcNAcbeta1-3Galbeta1-4Glcbeta-Cer; GalNAcalpha1-3(Fucalpha1-2)Galbeta1-3GalNAcalpha1-3(Fucalpha1-2)Galbeta1-4GlcNAcbeta1-3Galbeta1-4Glcbeta-Cer(d18:1/16:0); GalNAcalpha1-3(Fucalpha1-2)Galbeta1-3GalNAcalpha1-3(Fucalpha1-2)Galbeta1-4GlcNAcbeta1-3Galbeta1-4Glcbeta-Cer(d18:1/18:0); GalNAcalpha1-3(Fucalpha1-2)Galbeta1-3GalNAcalpha1-3(Fucalpha1-2)Galbeta1-4GlcNAcbeta1-3Galbeta1-4Glcbeta-Cer(d18:1/20:0); GalNAcalpha1-3(Fucalpha1-2)Galbeta1-3GalNAcalpha1-3(Fucalpha1-2)Galbeta1-4GlcNAcbeta1-3Galbeta1-4Glcbeta-Cer(d18:1/22:0); GalNAcalpha1-3(Fucalpha1-2)Galbeta1-3GalNAcalpha1-3(Fucalpha1-2)Galbeta1-4GlcNAcbeta1-3Galbeta1-4Glcbeta-Cer(d18:1/24:0); GalNAcalpha1-3(Fucalpha1-2)Galbeta1-3GalNAcalpha1-3(Fucalpha1-2)Galbeta1-4GlcNAcbeta1-3Galbeta1-4Glcbeta-Cer(d18:1/24:1(15Z)); GalNAcalpha1-3(Fucalpha1-2)Galbeta1-3GalNAcalpha1-3(Fucalpha1-2)Galbeta1-4GlcNAcbeta1-3Galbeta1-4Glcbeta-Cer(d18:1/26:0); GalNAcalpha1-3(Fucalpha1-2)Galbeta1-3GalNAcalpha1-3(Fucalpha1-2)Galbeta1-4GlcNAcbeta1-3Galbeta1-4Glcbeta-Cer(d18:1/26:1(17Z)); GalNAcalpha1-3(Fucalpha1-2)Galbeta1-3GalNAcalpha1-3(Fucalpha1-2)Galbeta1-4GlcNAcbeta1-3Galbeta1-4GlcNAcbeta1-3Galbeta1-4Glcbeta-Cer; GalNAcalpha1-3(Fucalpha1-2)Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/16:0); GalNAcalpha1-3(Fucalpha1-2)Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/18:0); GalNAcalpha1-3(Fucalpha1-2)Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/20:0); GalNAcalpha1-3(Fucalpha1-2)Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/22:0); GalNAcalpha1-3(Fucalpha1-2)Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/24:0); GalNAcalpha1-3(Fucalpha1-2)Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/24:1(15Z)); GalNAcalpha1-3(Fucalpha1-2)Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/26:0); GalNAcalpha1-3(Fucalpha1-2)Galbeta1-3GalNAcalpha1-3(Fucalpha1-2)Galbeta1-4GlcNAcbeta1-3Galbeta1-4GlcNAcbeta1-3Galbeta1-4Glcbeta-Cer(d18:1/26:1(17Z)); GalNAcalpha1-3(Fucalpha1-2)Galbeta1-3GalNAcbeta1-3Galalpha1-4Galbeta1-4Glcbeta-Cer; GalNAcalpha1-3(Fucalpha1-2)Galbeta1-3GalNAcbeta1-3Galalpha1-4Galbeta1-4Glcbeta-Cer(d18:1/16:0); GalNAcalpha1-3(Fucalpha1-2)Galbeta1-3GalNAcbeta1-3Galalpha1-4Galbeta1-4Glcbeta-Cer(d18:1/18:0); GalNAcalpha1-3(Fucalpha1-2)Galbeta1-3GalNAcbeta1-3Galalpha1-4Galbeta1-4Glcbeta-Cer(d18:1/20:0); GalNAcalpha1-3(Fucalpha1-2)Galbeta1-3GalNAcbeta1-3Galalpha1-4Galbeta1-4Glcbeta-Cer(d18:1/22:0); GalNAcalpha1-3(Fucalpha1-2)Galbeta1-3GalNAcbeta1-3Galalpha1-4Galbeta1-4Glcbeta-Cer(d18:1/24:0); GalNAcalpha1-3(Fucalpha1-2)Galbeta1-3GalNAcbeta1-3Galalpha1-4Galbeta1-4Glcbeta-Cer(d18:1/24:1(15Z)); GalNAcalpha1-3(Fucalpha1-2)Galbeta1-3GalNAcbeta1-3Galalpha1-4Galbeta1-4Glcbeta-Cer(d18:1/26:0); GalNAcalpha1-3(Fucalpha1-2)Galbeta1-3GalNAcbeta1-3Galalpha1-4Galbeta1-4Glcbeta-Cer(d18:1/26:1(17Z)); GalNAcalpha1-3(Fucalpha1-2)Galbeta1-3GlcNAcbeta1-3(Fucalpha1-2Galbeta1-3GlcNAcbeta1-6)Galbeta1-3GlcNAcbeta1-3Galbeta1-4Glcbeta-Cer; GalNAcalpha1-3(Fucalpha1-2)Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/16:0); GalNAcalpha1-3(Fucalpha1-2)Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/18:0); GalNAcalpha1-3(Fucalpha1-2)Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/20:0); GalNAcalpha1-3(Fucalpha1-2)Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/22:0); GalNAcalpha1-3(Fucalpha1-2)Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/24:0); GalNAcalpha1-3(Fucalpha1-2)Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/24:1(15Z)); GalNAcalpha1-3(Fucalpha1-2)Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/26:0); GalNAcalpha1-3(Fucalpha1-2)Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/26:1(17Z)); GalNAcalpha1-3(Fucalpha1-2)Galbeta1-3GlcNAcbeta1-3(Fucalpha1-2Galbeta1-4GlcNAcbeta1-6)Galbeta1-3GlcNAcbeta1-3Galbeta1-4Glcbeta-Cer; GalNAcalpha1-3(Fucalpha1-2)Galbeta1-3GlcNAcbeta1-3(Fucalpha1-2Galbeta1-4GlcNAcbeta1-6)Galbeta1-3GlcNAcbeta1-3Galbeta1-4Glcbeta-Cer(d18:1/16:0); GalNAcalpha1-3(Fucalpha1-2)Galbeta1-3GlcNAcbeta1-3(Fucalpha1-2Galbeta1-4GlcNAcbeta1-6)Galbeta1-3GlcNAcbeta1-3Galbeta1-4Glcbeta-Cer(d18:1/18:0); GalNAcalpha1-3(Fucalpha1-2)Galbeta1-3GlcNAcbeta1-3(Fucalpha1-2Galbeta1-4GlcNAcbeta1-6)Galbeta1-3GlcNAcbeta1-3Galbeta1-4Glcbeta-Cer(d18:1/20:0); GalNAcalpha1-3(Fucalpha1-2)Galbeta1-3GlcNAcbeta1-3(Fucalpha1-2Galbeta1-4GlcNAcbeta1-6)Galbeta1-3GlcNAcbeta1-3Galbeta1-4Glcbeta-Cer(d18:1/22:0); GalNAcalpha1-3(Fucalpha1-2)Galbeta1-3GlcNAcbeta1-3(Fucalpha1-2Galbeta1-4GlcNAcbeta1-6)Galbeta1-3GlcNAcbeta1-3Galbeta1-4Glcbeta-Cer(d18:1/24:0); GalNAcalpha1-3(Fucalpha1-2)Galbeta1-3GlcNAcbeta1-3(Fucalpha1-2Galbeta1-4GlcNAcbeta1-6)Galbeta1-3GlcNAcbeta1-3Galbeta1-4Glcbeta-Cer(d18:1/24:1(15Z)); GalNAcalpha1-3(Fucalpha1-2)Galbeta1-3GlcNAcbeta1-3(Fucalpha1-2Galbeta1-4GlcNAcbeta1-6)Galbeta1-3GlcNAcbeta1-3Galbeta1-4Glcbeta-Cer(d18:1/26:0); GalNAcalpha1-3(Fucalpha1-2)Galbeta1-3GlcNAcbeta1-3(Fucalpha1-2Galbeta1-4GlcNAcbeta1-6)Galbeta1-3GlcNAcbeta1-3Galbeta1-4Glcbeta-Cer(d18:1/26:1(17Z)); GalNAcalpha1-3(Fucalpha1-2)Galbeta1-3GlcNAcbeta1-3(Galbeta1-3GlcNAcbeta1-6)Galbeta1-3GlcNAcbeta1-3Galbeta1-4Glcbeta-Cer; GalNAcalpha1-3(Fucalpha1-2)Galbeta1-3GlcNAcbeta1-3(Galbeta1-3GlcNAcbeta1-6)Galbeta1-3GlcNAcbeta1-3Galbeta1-4Glcbeta-Cer(d18:1/16:0); GalNAcalpha1-3(Fucalpha1-2)Galbeta1-3GlcNAcbeta1-3(Galbeta1-3GlcNAcbeta1-6)Galbeta1-3GlcNAcbeta1-3Galbeta1-4Glcbeta-Cer(d18:1/18:0); GalNAcalpha1-3(Fucalpha1-2)Galbeta1-3GlcNAcbeta1-3(Galbeta1-3GlcNAcbeta1-6)Galbeta1-3GlcNAcbeta1-3Galbeta1-4Glcbeta-Cer(d18:1/20:0); GalNAcalpha1-3(Fucalpha1-2)Galbeta1-3GlcNAcbeta1-3(Galbeta1-3GlcNAcbeta1-6)Galbeta1-3GlcNAcbeta1-3Galbeta1-4Glcbeta-Cer(d18:1/22:0); GalNAcalpha1-3(Fucalpha1-2)Galbeta1-3GlcNAcbeta1-3(Galbeta1-3GlcNAcbeta1-6)Galbeta1-3GlcNAcbeta1-3Galbeta1-4Glcbeta-Cer(d18:1/24:0); GalNAcalpha1-3(Fucalpha1-2)Galbeta1-3GlcNAcbeta1-3(Galbeta1-3GlcNAcbeta1-6)Galbeta1-3GlcNAcbeta1-3Galbeta1-4Glcbeta-Cer(d18:1/24:1(15Z)); GalNAcalpha1-3(Fucalpha1-2)Galbeta1-3GlcNAcbeta1-3(Galbeta1-3GlcNAcbeta1-6)Galbeta1-3GlcNAcbeta1-3Galbeta1-4Glcbeta-Cer(d18:1/26:0); GalNAcalpha1-3(Fucalpha1-2)Galbeta1-3GlcNAcbeta1-3(Galbeta1-3GlcNAcbeta1-6)Galbeta1-3GlcNAcbeta1-3Galbeta1-4Glcbeta-Cer(d18:1/26:1(17Z)); GalNAcalpha1-3(Fucalpha1-2)Galbeta1-3GlcNAcbeta1-3(GalNAcalpha1-3(Fucalpha1-2)Galbeta1-3GlcNAcbeta1-6)Galbeta1-3GlcNAcbeta1-3Galbeta1-4Glcbeta-Cer; GalNAcalpha1-3(Fucalpha1-2)Galbeta1-3GlcNAcbeta1-3(GalNAcalpha1-3(Fucalpha1-2)Galbeta1-3GlcNAcbeta1-6)Galbeta1-3GlcNAcbeta1-3Galbeta1-4Glcbeta-Cer(d18:1/16:0); GalNAcalpha1-3(Fucalpha1-2)Galbeta1-3GlcNAcbeta1-3(GalNAcalpha1-3(Fucalpha1-2)Galbeta1-3GlcNAcbeta1-6)Galbeta1-3GlcNAcbeta1-3Galbeta1-4Glcbeta-Cer(d18:1/18:0); GalNAcalpha1-3(Fucalpha1-2)Galbeta1-3GlcNAcbeta1-3(GalNAcalpha1-3(Fucalpha1-2)Galbeta1-3GlcNAcbeta1-6)Galbeta1-3GlcNAcbeta1-3Galbeta1-4Glcbeta-Cer(d18:1/20:0); GalNAcalpha1-3(Fucalpha1-2)Galbeta1-3GlcNAcbeta1-3(GalNAcalpha1-3(Fucalpha1-2)Galbeta1-3GlcNAcbeta1-6)Galbeta1-3GlcNAcbeta1-3Galbeta1-4Glcbeta-Cer(d18:1/22:0); GalNAcalpha1-3(Fucalpha1-2)Galbeta1-3GlcNAcbeta1-3(GalNAcalpha1-3(Fucalpha1-2)Galbeta1-3GlcNAcbeta1-6)Galbeta1-3GlcNAcbeta1-3Galbeta1-4Glcbeta-Cer(d18:1/24:0); GalNAcalpha1-3(Fucalpha1-2)Galbeta1-3GlcNAcbeta1-3(GalNAcalpha1-3(Fucalpha1-2)Galbeta1-3GlcNAcbeta1-6)Galbeta1-3GlcNAcbeta1-3Galbeta1-4Glcbeta-Cer(d18:1/24:1(15Z)); GalNAcalpha1-3(Fucalpha1-2)Galbeta1-3GlcNAcbeta1-3(GalNAcalpha1-3(Fucalpha1-2)Galbeta1-3GlcNAcbeta1-6)Galbeta1-3GlcNAcbeta1-3Galbeta1-4Glcbeta-Cer(d18:1/26:0); GalNAcalpha1-3(Fucalpha1-2)Galbeta1-3GlcNAcbeta1-3(GalNAcalpha1-3(Fucalpha1-2)Galbeta1-3GlcNAcbeta1-6)Galbeta1-3GlcNAcbeta1-3Galbeta1-4Glcbeta-Cer(d18:1/26:1(17Z)); GalNAcalpha1-3(Fucalpha1-2)Galbeta1-3GlcNAcbeta1-3(GalNAcalpha1-3Galbeta1-3GlcNAcbeta1-6)Galbeta1-3GlcNAcbeta1-3Galbeta1-4Glcbeta-Cer; GalNAcalpha1-3(Fucalpha1-2)Galbeta1-3GlcNAcbeta1-3(GalNAcalpha1-3Galbeta1-3GlcNAcbeta1-6)Galbeta1-3GlcNAcbeta1-3Galbeta1-4Glcbeta-Cer(d18:1/16:0); GalNAcalpha1-3(Fucalpha1-2)Galbeta1-3GlcNAcbeta1-3(GalNAcalpha1-3Galbeta1-3GlcNAcbeta1-6)Galbeta1-3GlcNAcbeta1-3Galbeta1-4Glcbeta-Cer(d18:1/18:0); GalNAcalpha1-3(Fucalpha1-2)Galbeta1-3GlcNAcbeta1-3(GalNAcalpha1-3Galbeta1-3GlcNAcbeta1-6)Galbeta1-3GlcNAcbeta1-3Galbeta1-4Glcbeta-Cer(d18:1/20:0); GalNAcalpha1-3(Fucalpha1-2)Galbeta1-3GlcNAcbeta1-3(GalNAcalpha1-3Galbeta1-3GlcNAcbeta1-6)Galbeta1-3GlcNAcbeta1-3Galbeta1-4Glcbeta-Cer(d18:1/22:0); GalNAcalpha1-3(Fucalpha1-2)Galbeta1-3GlcNAcbeta1-3(GalNAcalpha1-3Galbeta1-3GlcNAcbeta1-6)Galbeta1-3GlcNAcbeta1-3Galbeta1-4Glcbeta-Cer(d18:1/24:0); GalNAcalpha1-3(Fucalpha1-2)Galbeta1-3GlcNAcbeta1-3(GalNAcalpha1-3Galbeta1-3GlcNAcbeta1-6)Galbeta1-3GlcNAcbeta1-3Galbeta1-4Glcbeta-Cer(d18:1/24:1(15Z)); GalNAcalpha1-3(Fucalpha1-2)Galbeta1-3GlcNAcbeta1-3(GalNAcalpha1-3Galbeta1-3GlcNAcbeta1-6)Galbeta1-3GlcNAcbeta1-3Galbeta1-4Glcbeta-Cer(d18:1/26:0); GalNAcalpha1-3(Fucalpha1-2)Galbeta1-3GlcNAcbeta1-3(GalNAcalpha1-3Galbeta1-3GlcNAcbeta1-6)Galbeta1-3GlcNAcbeta1-3Galbeta1-4Glcbeta-Cer(d18:1/26:1(17Z)); GalNAcalpha1-3(Fucalpha1-2)Galbeta1-3GlcNAcbeta1-3(GlcNAcalpha1-3(Fucalpha1-2)Galbeta1-4GlcNAcbeta1-6)Galbeta1-3GlcNAcbeta1-3Galbeta1-4Glcbeta-Cer; GalNAcalpha1-3(Fucalpha1-2)Galbeta1-3GlcNAcbeta1-3(GlcNAcalpha1-3(Fucalpha1-2)Galbeta1-4GlcNAcbeta1-6)Galbeta1-3GlcNAcbeta1-3Galbeta1-4Glcbeta-Cer(d18:1/16:0); GalNAcalpha1-3(Fucalpha1-2)Galbeta1-3GlcNAcbeta1-3(GlcNAcalpha1-3(Fucalpha1-2)Galbeta1-4GlcNAcbeta1-6)Galbeta1-3GlcNAcbeta1-3Galbeta1-4Glcbeta-Cer(d18:1/18:0); GalNAcalpha1-3(Fucalpha1-2)Galbeta1-3GlcNAcbeta1-3(GlcNAcalpha1-3(Fucalpha1-2)Galbeta1-4GlcNAcbeta1-6)Galbeta1-3GlcNAcbeta1-3Galbeta1-4Glcbeta-Cer(d18:1/20:0); GalNAcalpha1-3(Fucalpha1-2)Galbeta1-3GlcNAcbeta1-3(GlcNAcalpha1-3(Fucalpha1-2)Galbeta1-4GlcNAcbeta1-6)Galbeta1-3GlcNAcbeta1-3Galbeta1-4Glcbeta-Cer(d18:1/22:0); GalNAcalpha1-3(Fucalpha1-2)Galbeta1-3GlcNAcbeta1-3(GlcNAcalpha1-3(Fucalpha1-2)Galbeta1-4GlcNAcbeta1-6)Galbeta1-3GlcNAcbeta1-3Galbeta1-4Glcbeta-Cer(d18:1/24:0); GalNAcalpha1-3(Fucalpha1-2)Galbeta1-3GlcNAcbeta1-3(GlcNAcalpha1-3(Fucalpha1-2)Galbeta1-4GlcNAcbeta1-6)Galbeta1-3GlcNAcbeta1-3Galbeta1-4Glcbeta-Cer(d18:1/24:1(15Z)); GalNAcalpha1-3(Fucalpha1-2)Galbeta1-3GlcNAcbeta1-3(GlcNAcalpha1-3(Fucalpha1-2)Galbeta1-4GlcNAcbeta1-6)Galbeta1-3GlcNAcbeta1-3Galbeta1-4Glcbeta-Cer(d18:1/26:0); GalNAcalpha1-3(Fucalpha1-2)Galbeta1-3GlcNAcbeta1-3(GlcNAcalpha1-3(Fucalpha1-2)Galbeta1-4GlcNAcbeta1-6)Galbeta1-3GlcNAcbeta1-3Galbeta1-4Glcbeta-Cer(d18:1/26:1(17Z)); GalNAcalpha1-3(Fucalpha1-2)Galbeta1-3GlcNAcbeta1-3(GlcNAcbeta1-6)Galbeta1-3GlcNAcbeta1-3Galbeta1-4Glcbeta-Cer; GalNAcalpha1-3(Fucalpha1-2)Galbeta1-3GlcNAcbeta1-3(GlcNAcbeta1-6)Galbeta1-3GlcNAcbeta1-3Galbeta1-4Glcbeta-Cer(d18:1/16:0); GalNAcalpha1-3(Fucalpha1-2)Galbeta1-3GlcNAcbeta1-3(GlcNAcbeta1-6)Galbeta1-3GlcNAcbeta1-3Galbeta1-4Glcbeta-Cer(d18:1/18:0); GalNAcalpha1-3(Fucalpha1-2)Galbeta1-3GlcNAcbeta1-3(GlcNAcbeta1-6)Galbeta1-3GlcNAcbeta1-3Galbeta1-4Glcbeta-Cer(d18:1/20:0); GalNAcalpha1-3(Fucalpha1-2)Galbeta1-3GlcNAcbeta1-3(GlcNAcbeta1-6)Galbeta1-3GlcNAcbeta1-3Galbeta1-4Glcbeta-Cer(d18:1/22:0); GalNAcalpha1-3(Fucalpha1-2)Galbeta1-3GlcNAcbeta1-3(GlcNAcbeta1-6)Galbeta1-3GlcNAcbeta1-3Galbeta1-4Glcbeta-Cer(d18:1/24:0); GalNAcalpha1-3(Fucalpha1-2)Galbeta1-3GlcNAcbeta1-3(GlcNAcbeta1-6)Galbeta1-3GlcNAcbeta1-3Galbeta1-4Glcbeta-Cer(d18:1/24:1(15Z)); GalNAcalpha1-3(Fucalpha1-2)Galbeta1-3GlcNAcbeta1-3(GlcNAcbeta1-6)Galbeta1-3GlcNAcbeta1-3Galbeta1-4Glcbeta-Cer(d18:1/26:0); GalNAcalpha1-3(Fucalpha1-2)Galbeta1-3GlcNAcbeta1-3(GlcNAcbeta1-6)Galbeta1-3GlcNAcbeta1-3Galbeta1-4Glcbeta-Cer(d18:1/26:1(17Z)); GalNAcalpha1-3(Fucalpha1-2)Galbeta1-3GlcNAcbeta1-3Galbeta1-3GlcNAcbeta1-3Galbeta1-4Glcbeta-Cer; GalNAcalpha1-3(Fucalpha1-2)Galbeta1-3GlcNAcbeta1-3Galbeta1-3GlcNAcbeta1-3Galbeta1-4Glcbeta-Cer(d18:1/16:0); GalNAcalpha1-3(Fucalpha1-2)Galbeta1-3GlcNAcbeta1-3Galbeta1-3GlcNAcbeta1-3Galbeta1-4Glcbeta-Cer(d18:1/18:0); GalNAcalpha1-3(Fucalpha1-2)Galbeta1-3GlcNAcbeta1-3Galbeta1-3GlcNAcbeta1-3Galbeta1-4Glcbeta-Cer(d18:1/20:0); GalNAcalpha1-3(Fucalpha1-2)Galbeta1-3GlcNAcbeta1-3Galbeta1-3GlcNAcbeta1-3Galbeta1-4Glcbeta-Cer(d18:1/22:0); GalNAcalpha1-3(Fucalpha1-2)Galbeta1-3GlcNAcbeta1-3Galbeta1-3GlcNAcbeta1-3Galbeta1-4Glcbeta-Cer(d18:1/24:0); GalNAcalpha1-3(Fucalpha1-2)Galbeta1-3GlcNAcbeta1-3Galbeta1-3GlcNAcbeta1-3Galbeta1-4Glcbeta-Cer(d18:1/24:1(15Z)); GalNAcalpha1-3(Fucalpha1-2)Galbeta1-3GlcNAcbeta1-3Galbeta1-3GlcNAcbeta1-3Galbeta1-4Glcbeta-Cer(d18:1/26:0); GalNAcalpha1-3(Fucalpha1-2)Galbeta1-3GlcNAcbeta1-3Galbeta1-3GlcNAcbeta1-3Galbeta1-4Glcbeta-Cer(d18:1/26:1(17Z)); GalNAcalpha1-3(Fucalpha1-2)Galbeta1-3GlcNAcbeta1-3Galbeta1-4Glcbeta-Cer; GalNAcalpha1-3(Fucalpha1-2)Galbeta1-3GlcNAcbeta1-3Galbeta1-4Glcbeta-Cer(d18:1/16:0); GalNAcalpha1-3(Fucalpha1-2)Galbeta1-3GlcNAcbeta1-3Galbeta1-4Glcbeta-Cer(d18:1/18:0); GalNAcalpha1-3(Fucalpha1-2)Galbeta1-3GlcNAcbeta1-3Galbeta1-4Glcbeta-Cer(d18:1/20:0); GalNAcalpha1-3(Fucalpha1-2)Galbeta1-3GlcNAcbeta1-3Galbeta1-4Glcbeta-Cer(d18:1/22:0); GalNAcalpha1-3(Fucalpha1-2)Galbeta1-3GlcNAcbeta1-3Galbeta1-4Glcbeta-Cer(d18:1/24:0); GalNAcalpha1-3(Fucalpha1-2)Galbeta1-3GlcNAcbeta1-3Galbeta1-4Glcbeta-Cer(d18:1/24:1(15Z)); GalNAcalpha1-3(Fucalpha1-2)Galbeta1-3GlcNAcbeta1-3Galbeta1-4Glcbeta-Cer(d18:1/26:0); GalNAcalpha1-3(Fucalpha1-2)Galbeta1-3GlcNAcbeta1-3Galbeta1-4Glcbeta-Cer(d18:1/26:1(17Z)); GalNAcalpha1-3(Fucalpha1-2)Galbeta1-4(Fucalpha1-3)GlcNAcbeta1-3Galbeta1-4Glcbeta-Cer; GalNAcalpha1-3(Fucalpha1-2)Galbeta1-4(Fucalpha1-3)GlcNAcbeta1-3Galbeta1-4Glcbeta-Cer(d18:1/16:0); GalNAcalpha1-3(Fucalpha1-2)Galbeta1-4(Fucalpha1-3)GlcNAcbeta1-3Galbeta1-4Glcbeta-Cer(d18:1/18:0); GalNAcalpha1-3(Fucalpha1-2)Galbeta1-4(Fucalpha1-3)GlcNAcbeta1-3Galbeta1-4Glcbeta-Cer(d18:1/20:0); GalNAcalpha1-3(Fucalpha1-2)Galbeta1-4(Fucalpha1-3)GlcNAcbeta1-3Galbeta1-4Glcbeta-Cer(d18:1/22:0); GalNAcalpha1-3(Fucalpha1-2)Galbeta1-4(Fucalpha1-3)GlcNAcbeta1-3Galbeta1-4Glcbeta-Cer(d18:1/24:0); GalNAcalpha1-3(Fucalpha1-2)Galbeta1-4(Fucalpha1-3)GlcNAcbeta1-3Galbeta1-4Glcbeta-Cer(d18:1/24:1(15Z)); GalNAcalpha1-3(Fucalpha1-2)Galbeta1-4(Fucalpha1-3)GlcNAcbeta1-3Galbeta1-4Glcbeta-Cer(d18:1/26:0); GalNAcalpha1-3(Fucalpha1-2)Galbeta1-4(Fucalpha1-3)GlcNAcbeta1-3Galbeta1-4Glcbeta-Cer(d18:1/26:1(17Z)); GalNAcalpha1-3(Fucalpha1-2)Galbeta1-4GlcNAcbeta1-3(GalNAcalpha1-3(Fucalpha1-2)Galbeta1-4GlcNAcbeta1-6)Galbeta1-4GlcNAcbeta1-3Galbeta1-4Glcbeta-Cer; GalNAcalpha1-3(Fucalpha1-2)Galbeta1-4GlcNAcbeta1-3(GalNAcalpha1-3(Fucalpha1-2)Galbeta1-4GlcNAcbeta1-6)Galbeta1-4GlcNAcbeta1-3Galbeta1-4Glcbeta-Cer(d18:1/16:0); GalNAcalpha1-3(Fucalpha1-2)Galbeta1-4GlcNAcbeta1-3(GalNAcalpha1-3(Fucalpha1-2)Galbeta1-4GlcNAcbeta1-6)Galbeta1-4GlcNAcbeta1-3Galbeta1-4Glcbeta-Cer(d18:1/18:0); GalNAcalpha1-3(Fucalpha1-2)Galbeta1-4GlcNAcbeta1-3(GalNAcalpha1-3(Fucalpha1-2)Galbeta1-4GlcNAcbeta1-6)Galbeta1-4GlcNAcbeta1-3Galbeta1-4Glcbeta-Cer(d18:1/20:0); GalNAcalpha1-3(Fucalpha1-2)Galbeta1-4GlcNAcbeta1-3(GalNAcalpha1-3(Fucalpha1-2)Galbeta1-4GlcNAcbeta1-6)Galbeta1-4GlcNAcbeta1-3Galbeta1-4Glcbeta-Cer(d18:1/22:0); GalNAcalpha1-3(Fucalpha1-2)Galbeta1-4GlcNAcbeta1-3(GalNAcalpha1-3(Fucalpha1-2)Galbeta1-4GlcNAcbeta1-6)Galbeta1-4GlcNAcbeta1-3Galbeta1-4Glcbeta-Cer(d18:1/24:0); GalNAcalpha1-3(Fucalpha1-2)Galbeta1-4GlcNAcbeta1-3(GalNAcalpha1-3(Fucalpha1-2)Galbeta1-4GlcNAcbeta1-6)Galbeta1-4GlcNAcbeta1-3Galbeta1-4Glcbeta-Cer(d18:1/24:1(15Z)); GalNAcalpha1-3(Fucalpha1-2)Galbeta1-4GlcNAcbeta1-3(GalNAcalpha1-3(Fucalpha1-2)Galbeta1-4GlcNAcbeta1-6)Galbeta1-4GlcNAcbeta1-3Galbeta1-4Glcbeta-Cer(d18:1/26:0); GalNAcalpha1-3(Fucalpha1-2)Galbeta1-4GlcNAcbeta1-3(GalNAcalpha1-3(Fucalpha1-2)Galbeta1-4GlcNAcbeta1-6)Galbeta1-4GlcNAcbeta1-3Galbeta1-4Glcbeta-Cer(d18:1/26:1(17Z)); GalNAcalpha1-3(Fucalpha1-2)Galbeta1-4GlcNAcbeta1-3Galbeta1-4Glcbeta-Cer; GalNAcalpha1-3(Fucalpha1-2)Galbeta1-4GlcNAcbeta1-3Galbeta1-4Glcbeta-Cer(d18:1/16:0); GalNAcalpha1-3(Fucalpha1-2)Galbeta1-4GlcNAcbeta1-3Galbeta1-4Glcbeta-Cer(d18:1/18:0); GalNAcalpha1-3(Fucalpha1-2)Galbeta1-4GlcNAcbeta1-3Galbeta1-4Glcbeta-Cer(d18:1/20:0); GalNAcalpha1-3(Fucalpha1-2)Galbeta1-4GlcNAcbeta1-3Galbeta1-4Glcbeta-Cer(d18:1/22:0); GalNAcalpha1-3(Fucalpha1-2)Galbeta1-4GlcNAcbeta1-3Galbeta1-4Glcbeta-Cer(d18:1/24:0); GalNAcalpha1-3(Fucalpha1-2)Galbeta1-4GlcNAcbeta1-3Galbeta1-4Glcbeta-Cer(d18:1/24:1(15Z)); GalNAcalpha1-3(Fucalpha1-2)Galbeta1-4GlcNAcbeta1-3Galbeta1-4Glcbeta-Cer(d18:1/26:0); GalNAcalpha1-3(Fucalpha1-2)Galbeta1-4GlcNAcbeta1-3Galbeta1-4Glcbeta-Cer(d18:1/26:1(17Z)); GalNAcalpha1-3(Fucalpha1-2)Galbeta1-4GlcNAcbeta1-3Galbeta1-4GlcNAcbeta1-3Galbeta1-4Glcbeta-Cer; GalNAcalpha1-3(Fucalpha1-2)Galbeta1-4GlcNAcbeta1-3Galbeta1-4GlcNAcbeta1-3Galbeta1-4Glcbeta-Cer(d18:1/16:0); GalNAcalpha1-3(Fucalpha1-2)Galbeta1-4GlcNAcbeta1-3Galbeta1-4GlcNAcbeta1-3Galbeta1-4Glcbeta-Cer(d18:1/18:0); GalNAcalpha1-3(Fucalpha1-2)Galbeta1-4GlcNAcbeta1-3Galbeta1-4GlcNAcbeta1-3Galbeta1-4Glcbeta-Cer(d18:1/20:0); GalNAcalpha1-3(Fucalpha1-2)Galbeta1-4GlcNAcbeta1-3Galbeta1-4GlcNAcbeta1-3Galbeta1-4Glcbeta-Cer(d18:1/22:0); GalNAcalpha1-3(Fucalpha1-2)Galbeta1-4GlcNAcbeta1-3Galbeta1-4GlcNAcbeta1-3Galbeta1-4Glcbeta-Cer(d18:1/24:0); GalNAcalpha1-3(Fucalpha1-2)Galbeta1-4GlcNAcbeta1-3Galbeta1-4GlcNAcbeta1-3Galbeta1-4Glcbeta-Cer(d18:1/24:1(15Z)); GalNAcalpha1-3(Fucalpha1-2)Galbeta1-4GlcNAcbeta1-3Galbeta1-4GlcNAcbeta1-3Galbeta1-4Glcbeta-Cer(d18:1/26:0); GalNAcalpha1-3(Fucalpha1-2)Galbeta1-4GlcNAcbeta1-3Galbeta1-4GlcNAcbeta1-3Galbeta1-4Glcbeta-Cer(d18:1/26:1(17Z)); GalNAcalpha1-3(Fucalpha1-2)Galbeta1-4GlcNAcbeta1-3Galbeta1-4GlcNAcbeta1-3Galbeta1-4GlcNAcbeta1-3Galbeta1-4Glcbeta-Cer; GalNAcalpha1-3(Fucalpha1-2)Galbeta1-4GlcNAcbeta1-3Galbeta1-4GlcNAcbeta1-3Galbeta1-4GlcNAcbeta1-3Galbeta1-4Glcbeta-Cer(d18:1/16:0); GalNAcalpha1-3(Fucalpha1-2)Galbeta1-4GlcNAcbeta1-3Galbeta1-4GlcNAcbeta1-3Galbeta1-4GlcNAcbeta1-3Galbeta1-4Glcbeta-Cer(d18:1/18:0); GalNAcalpha1-3(Fucalpha1-2)Galbeta1-4GlcNAcbeta1-3Galbeta1-4GlcNAcbeta1-3Galbeta1-4GlcNAcbeta1-3Galbeta1-4Glcbeta-Cer(d18:1/20:0); GalNAcalpha1-3(Fucalpha1-2)Galbeta1-4GlcNAcbeta1-3Galbeta1-4GlcNAcbeta1-3Galbeta1-4GlcNAcbeta1-3Galbeta1-4Glcbeta-Cer(d18:1/22:0); GalNAcalpha1-3(Fucalpha1-2)Galbeta1-4GlcNAcbeta1-3Galbeta1-4GlcNAcbeta1-3Galbeta1-4GlcNAcbeta1-3Galbeta1-4Glcbeta-Cer(d18:1/24:0); GalNAcalpha1-3(Fucalpha1-2)Galbeta1-4GlcNAcbeta1-3Galbeta1-4GlcNAcbeta1-3Galbeta1-4GlcNAcbeta1-3Galbeta1-4Glcbeta-Cer(d18:1/24:1(15Z)); GalNAcalpha1-3(Fucalpha1-2)Galbeta1-4GlcNAcbeta1-3Galbeta1-4GlcNAcbeta1-3Galbeta1-4GlcNAcbeta1-3Galbeta1-4Glcbeta-Cer(d18:1/26:0); GalNAcalpha1-3(Fucalpha1-2)Galbeta1-4GlcNAcbeta1-3Galbeta1-4GlcNAcbeta1-3Galbeta1-4GlcNAcbeta1-3Galbeta1-4Glcbeta-Cer(d18:1/26:1(17Z)); GalNAcalpha1-3Galbeta1-3GalNAcbeta1-3Galalpha1-4Galbeta1-4Glcbeta-Cer; GalNAcalpha1-3Galbeta1-3GalNAcbeta1-3Galalpha1-4Galbeta1-4Glcbeta-Cer(d18:1/16:0); GalNAcalpha1-3Galbeta1-3GalNAcbeta1-3Galalpha1-4Galbeta1-4Glcbeta-Cer(d18:1/18:0); GalNAcalpha1-3Galbeta1-3GalNAcbeta1-3Galalpha1-4Galbeta1-4Glcbeta-Cer(d18:1/20:0); GalNAcalpha1-3Galbeta1-3GalNAcbeta1-3Galalpha1-4Galbeta1-4Glcbeta-Cer(d18:1/22:0); GalNAcalpha1-3Galbeta1-3GalNAcbeta1-3Galalpha1-4Galbeta1-4Glcbeta-Cer(d18:1/24:0); GalNAcalpha1-3Galbeta1-3GalNAcbeta1-3Galalpha1-4Galbeta1-4Glcbeta-Cer(d18:1/24:1(15Z)); GalNAcalpha1-3Galbeta1-3GalNAcbeta1-3Galalpha1-4Galbeta1-4Glcbeta-Cer(d18:1/26:0); GalNAcalpha1-3Galbeta1-3GalNAcbeta1-3Galalpha1-4Galbeta1-4Glcbeta-Cer(d18:1/26:1(17Z)); GalNAcalpha1-3Galbeta1-3GlcNAcbeta1-3(Fucalpha1-2Galbeta1-3GlcNAcbeta1-6)Galbeta1-3GlcNAcbeta1-3Galbeta1-4Glcbeta-Cer; GalNAcalpha1-3Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/16:0); GalNAcalpha1-3Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/18:0); GalNAcalpha1-3Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/20:0); GalNAcalpha1-3Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/22:0); GalNAcalpha1-3Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/24:0); GalNAcalpha1-3Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/24:1(15Z)); GalNAcalpha1-3Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/26:0); GalNAcalpha1-3Galbeta1-3GlcNAcbeta1-3(Fucalpha1-2Galbeta1-3GlcNAcbeta1-6)Galbeta1-3GlcNAcbeta1-3Galbeta1-4Glcbeta-Cer(d18:1/26:1(17Z)); GalNAcalpha1-3Galbeta1-3GlcNAcbeta1-3(Galbeta1-3GlcNAcbeta1-6)Galbeta1-3GlcNAcbeta1-3Galbeta1-4Glcbeta-Cer; GalNAcalpha1-3Galbeta1-3GlcNAcbeta1-3(Galbeta1-3GlcNAcbeta1-6)Galbeta1-3GlcNAcbeta1-3Galbeta1-4Glcbeta-Cer(d18:1/16:0); GalNAcalpha1-3Galbeta1-3GlcNAcbeta1-3(Galbeta1-3GlcNAcbeta1-6)Galbeta1-3GlcNAcbeta1-3Galbeta1-4Glcbeta-Cer(d18:1/18:0); GalNAcalpha1-3Galbeta1-3GlcNAcbeta1-3(Galbeta1-3GlcNAcbeta1-6)Galbeta1-3GlcNAcbeta1-3Galbeta1-4Glcbeta-Cer(d18:1/20:0); GalNAcalpha1-3Galbeta1-3GlcNAcbeta1-3(Galbeta1-3GlcNAcbeta1-6)Galbeta1-3GlcNAcbeta1-3Galbeta1-4Glcbeta-Cer(d18:1/22:0); GalNAcalpha1-3Galbeta1-3GlcNAcbeta1-3(Galbeta1-3GlcNAcbeta1-6)Galbeta1-3GlcNAcbeta1-3Galbeta1-4Glcbeta-Cer(d18:1/24:0); GalNAcalpha1-3Galbeta1-3GlcNAcbeta1-3(Galbeta1-3GlcNAcbeta1-6)Galbeta1-3GlcNAcbeta1-3Galbeta1-4Glcbeta-Cer(d18:1/24:1(15Z)); GalNAcalpha1-3Galbeta1-3GlcNAcbeta1-3(Galbeta1-3GlcNAcbeta1-6)Galbeta1-3GlcNAcbeta1-3Galbeta1-4Glcbeta-Cer(d18:1/26:0); GalNAcalpha1-3Galbeta1-3GlcNAcbeta1-3(Galbeta1-3GlcNAcbeta1-6)Galbeta1-3GlcNAcbeta1-3Galbeta1-4Glcbeta-Cer(d18:1/26:1(17Z)); GalNAcalpha1-3Galbeta1-3GlcNAcbeta1-3(GalNAcalpha1-3(Fucalpha1-2)Galbeta1-3GlcNAcbeta1-6)Galbeta1-3GlcNAcbeta1-3Galbeta1-4Glcbeta-Cer; GalNAcalpha1-3Galbeta1-3GlcNAcbeta1-3(GalNAcalpha1-3(Fucalpha1-2)Galbeta1-3GlcNAcbeta1-6)Galbeta1-3GlcNAcbeta1-3Galbeta1-4Glcbeta-Cer(d18:1/16:0); GalNAcalpha1-3Galbeta1-3GlcNAcbeta1-3(GalNAcalpha1-3(Fucalpha1-2)Galbeta1-3GlcNAcbeta1-6)Galbeta1-3GlcNAcbeta1-3Galbeta1-4Glcbeta-Cer(d18:1/18:0); GalNAcalpha1-3Galbeta1-3GlcNAcbeta1-3(GalNAcalpha1-3(Fucalpha1-2)Galbeta1-3GlcNAcbeta1-6)Galbeta1-3GlcNAcbeta1-3Galbeta1-4Glcbeta-Cer(d18:1/20:0); GalNAcalpha1-3Galbeta1-3GlcNAcbeta1-3(GalNAcalpha1-3(Fucalpha1-2)Galbeta1-3GlcNAcbeta1-6)Galbeta1-3GlcNAcbeta1-3Galbeta1-4Glcbeta-Cer(d18:1/22:0); GalNAcalpha1-3Galbeta1-3GlcNAcbeta1-3(GalNAcalpha1-3(Fucalpha1-2)Galbeta1-3GlcNAcbeta1-6)Galbeta1-3GlcNAcbeta1-3Galbeta1-4Glcbeta-Cer(d18:1/24:0); GalNAcalpha1-3Galbeta1-3GlcNAcbeta1-3(GalNAcalpha1-3(Fucalpha1-2)Galbeta1-3GlcNAcbeta1-6)Galbeta1-3GlcNAcbeta1-3Galbeta1-4Glcbeta-Cer(d18:1/24:1(15Z)); GalNAcalpha1-3Galbeta1-3GlcNAcbeta1-3(GalNAcalpha1-3(Fucalpha1-2)Galbeta1-3GlcNAcbeta1-6)Galbeta1-3GlcNAcbeta1-3Galbeta1-4Glcbeta-Cer(d18:1/26:0); GalNAcalpha1-3Galbeta1-3GlcNAcbeta1-3(GalNAcalpha1-3(Fucalpha1-2)Galbeta1-3GlcNAcbeta1-6)Galbeta1-3GlcNAcbeta1-3Galbeta1-4Glcbeta-Cer(d18:1/26:1(17Z)); GalNAcalpha1-3Galbeta1-3GlcNAcbeta1-3(GalNAcalpha1-3Galbeta1-3GlcNAcbeta1-6)Galbeta1-3GlcNAcbeta1-3Galbeta1-4Glcbeta-Cer; GalNAcalpha1-3Galbeta1-3GlcNAcbeta1-3(GalNAcalpha1-3Galbeta1-3GlcNAcbeta1-6)Galbeta1-3GlcNAcbeta1-3Galbeta1-4Glcbeta-Cer(d18:1/16:0); GalNAcalpha1-3Galbeta1-3GlcNAcbeta1-3(GalNAcalpha1-3Galbeta1-3GlcNAcbeta1-6)Galbeta1-3GlcNAcbeta1-3Galbeta1-4Glcbeta-Cer(d18:1/18:0); GalNAcalpha1-3Galbeta1-3GlcNAcbeta1-3(GalNAcalpha1-3Galbeta1-3GlcNAcbeta1-6)Galbeta1-3GlcNAcbeta1-3Galbeta1-4Glcbeta-Cer(d18:1/20:0); GalNAcalpha1-3Galbeta1-3GlcNAcbeta1-3(GalNAcalpha1-3Galbeta1-3GlcNAcbeta1-6)Galbeta1-3GlcNAcbeta1-3Galbeta1-4Glcbeta-Cer(d18:1/22:0); GalNAcalpha1-3Galbeta1-3GlcNAcbeta1-3(GalNAcalpha1-3Galbeta1-3GlcNAcbeta1-6)Galbeta1-3GlcNAcbeta1-3Galbeta1-4Glcbeta-Cer(d18:1/24:0); GalNAcalpha1-3Galbeta1-3GlcNAcbeta1-3(GalNAcalpha1-3Galbeta1-3GlcNAcbeta1-6)Galbeta1-3GlcNAcbeta1-3Galbeta1-4Glcbeta-Cer(d18:1/24:1(15Z)); GalNAcalpha1-3Galbeta1-3GlcNAcbeta1-3(GalNAcalpha1-3Galbeta1-3GlcNAcbeta1-6)Galbeta1-3GlcNAcbeta1-3Galbeta1-4Glcbeta-Cer(d18:1/26:0); GalNAcalpha1-3Galbeta1-3GlcNAcbeta1-3(GalNAcalpha1-3Galbeta1-3GlcNAcbeta1-6)Galbeta1-3GlcNAcbeta1-3Galbeta1-4Glcbeta-Cer(d18:1/26:1(17Z)); GalNAcalpha1-3Galbeta1-3GlcNAcbeta1-3(GlcNAcbeta1-6)Galbeta1-3GlcNAcbeta1-3Galbeta1-4Glcbeta-Cer; GalNAcalpha1-3Galbeta1-3GlcNAcbeta1-3(GlcNAcbeta1-6)Galbeta1-3GlcNAcbeta1-3Galbeta1-4Glcbeta-Cer(d18:1/16:0); GalNAcalpha1-3Galbeta1-3GlcNAcbeta1-3(GlcNAcbeta1-6)Galbeta1-3GlcNAcbeta1-3Galbeta1-4Glcbeta-Cer(d18:1/18:0); GalNAcalpha1-3Galbeta1-3GlcNAcbeta1-3(GlcNAcbeta1-6)Galbeta1-3GlcNAcbeta1-3Galbeta1-4Glcbeta-Cer(d18:1/20:0); GalNAcalpha1-3Galbeta1-3GlcNAcbeta1-3(GlcNAcbeta1-6)Galbeta1-3GlcNAcbeta1-3Galbeta1-4Glcbeta-Cer(d18:1/22:0); GalNAcalpha1-3Galbeta1-3GlcNAcbeta1-3(GlcNAcbeta1-6)Galbeta1-3GlcNAcbeta1-3Galbeta1-4Glcbeta-Cer(d18:1/24:0); GalNAcalpha1-3Galbeta1-3GlcNAcbeta1-3(GlcNAcbeta1-6)Galbeta1-3GlcNAcbeta1-3Galbeta1-4Glcbeta-Cer(d18:1/24:1(15Z)); GalNAcalpha1-3Galbeta1-3GlcNAcbeta1-3(GlcNAcbeta1-6)Galbeta1-3GlcNAcbeta1-3Galbeta1-4Glcbeta-Cer(d18:1/26:0); GalNAcalpha1-3Galbeta1-3GlcNAcbeta1-3(GlcNAcbeta1-6)Galbeta1-3GlcNAcbeta1-3Galbeta1-4Glcbeta-Cer(d18:1/26:1(17Z)); GalNAcalpha1-3Galbeta1-3GlcNAcbeta1-3Galbeta1-3GlcNAcbeta1-3Galbeta1-4Glcbeta-Cer; GalNAcalpha1-3Galbeta1-3GlcNAcbeta1-3Galbeta1-3GlcNAcbeta1-3Galbeta1-4Glcbeta-Cer(d18:1/16:0); GalNAcalpha1-3Galbeta1-3GlcNAcbeta1-3Galbeta1-3GlcNAcbeta1-3Galbeta1-4Glcbeta-Cer(d18:1/18:0); GalNAcalpha1-3Galbeta1-3GlcNAcbeta1-3Galbeta1-3GlcNAcbeta1-3Galbeta1-4Glcbeta-Cer(d18:1/20:0); GalNAcalpha1-3Galbeta1-3GlcNAcbeta1-3Galbeta1-3GlcNAcbeta1-3Galbeta1-4Glcbeta-Cer(d18:1/22:0); GalNAcalpha1-3Galbeta1-3GlcNAcbeta1-3Galbeta1-3GlcNAcbeta1-3Galbeta1-4Glcbeta-Cer(d18:1/24:0); GalNAcalpha1-3Galbeta1-3GlcNAcbeta1-3Galbeta1-3GlcNAcbeta1-3Galbeta1-4Glcbeta-Cer(d18:1/24:1(15Z)); GalNAcalpha1-3Galbeta1-3GlcNAcbeta1-3Galbeta1-3GlcNAcbeta1-3Galbeta1-4Glcbeta-Cer(d18:1/26:0); GalNAcalpha1-3Galbeta1-3GlcNAcbeta1-3Galbeta1-3GlcNAcbeta1-3Galbeta1-4Glcbeta-Cer(d18:1/26:1(17Z)); GalNAcalpha1-3Galbeta1-4GlcNAcbeta1-3(Fucalpha1-2(GalNAcalpha1-3)Galbeta1-4GlcNAcbeta1-6)Galbeta1-4GlcNAcbeta1-3Galbeta1-4Glcbeta-Cer; GalNAcalpha1-3Galbeta1-4GlcNAcbeta1-3(Fucalpha1-2(GalNAcalpha1-3)Galbeta1-4GlcNAcbeta1-6)Galbeta1-4GlcNAcbeta1-3Galbeta1-4Glcbeta-Cer(d18:1/16:0); GalNAcalpha1-3Galbeta1-4GlcNAcbeta1-3(Fucalpha1-2(GalNAcalpha1-3)Galbeta1-4GlcNAcbeta1-6)Galbeta1-4GlcNAcbeta1-3Galbeta1-4Glcbeta-Cer(d18:1/18:0); GalNAcalpha1-3Galbeta1-4GlcNAcbeta1-3(Fucalpha1-2(GalNAcalpha1-3)Galbeta1-4GlcNAcbeta1-6)Galbeta1-4GlcNAcbeta1-3Galbeta1-4Glcbeta-Cer(d18:1/20:0); GalNAcalpha1-3Galbeta1-4GlcNAcbeta1-3(Fucalpha1-2(GalNAcalpha1-3)Galbeta1-4GlcNAcbeta1-6)Galbeta1-4GlcNAcbeta1-3Galbeta1-4Glcbeta-Cer(d18:1/22:0); GalNAcalpha1-3Galbeta1-4GlcNAcbeta1-3(Fucalpha1-2(GalNAcalpha1-3)Galbeta1-4GlcNAcbeta1-6)Galbeta1-4GlcNAcbeta1-3Galbeta1-4Glcbeta-Cer(d18:1/24:0); GalNAcalpha1-3Galbeta1-4GlcNAcbeta1-3(Fucalpha1-2(GalNAcalpha1-3)Galbeta1-4GlcNAcbeta1-6)Galbeta1-4GlcNAcbeta1-3Galbeta1-4Glcbeta-Cer(d18:1/24:1(15Z)); GalNAcalpha1-3Galbeta1-4GlcNAcbeta1-3(Fucalpha1-2(GalNAcalpha1-3)Galbeta1-4GlcNAcbeta1-6)Galbeta1-4GlcNAcbeta1-3Galbeta1-4Glcbeta-Cer(d18:1/26:0); GalNAcalpha1-3Galbeta1-4GlcNAcbeta1-3(Fucalpha1-2(GalNAcalpha1-3)Galbeta1-4GlcNAcbeta1-6)Galbeta1-4GlcNAcbeta1-3Galbeta1-4Glcbeta-Cer(d18:1/26:1(17Z)); GalNAcalpha1-3Galbeta1-4GlcNAcbeta1-3(Fucalpha1-2Galbeta1-4GlcNAcbeta1-6)Galbeta1-4GlcNAcbeta1-3Galbeta1-4Glcbeta-Cer; GalNAcalpha1-3Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/16:0); GalNAcalpha1-3Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/18:0); GalNAcalpha1-3Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/20:0); GalNAcalpha1-3Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/22:0); GalNAcalpha1-3Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/24:0); GalNAcalpha1-3Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/24:1(15Z)); GalNAcalpha1-3Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/26:0); GalNAcalpha1-3Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/26:1(17Z)); GalNAcalpha1-3Galbeta1-4GlcNAcbeta1-3(Galbeta1-4GlcNAcbeta1-6)Galbeta1-4GlcNAcbeta1-3Galbeta1-4Glcbeta-Cer; GalNAcalpha1-3Galbeta1-4GlcNAcbeta1-3(Galbeta1-4GlcNAcbeta1-6)Galbeta1-4GlcNAcbeta1-3Galbeta1-4Glcbeta-Cer(d18:1/16:0); GalNAcalpha1-3Galbeta1-4GlcNAcbeta1-3(Galbeta1-4GlcNAcbeta1-6)Galbeta1-4GlcNAcbeta1-3Galbeta1-4Glcbeta-Cer(d18:1/18:0); GalNAcalpha1-3Galbeta1-4GlcNAcbeta1-3(Galbeta1-4GlcNAcbeta1-6)Galbeta1-4GlcNAcbeta1-3Galbeta1-4Glcbeta-Cer(d18:1/20:0); GalNAcalpha1-3Galbeta1-4GlcNAcbeta1-3(Galbeta1-4GlcNAcbeta1-6)Galbeta1-4GlcNAcbeta1-3Galbeta1-4Glcbeta-Cer(d18:1/22:0); GalNAcalpha1-3Galbeta1-4GlcNAcbeta1-3(Galbeta1-4GlcNAcbeta1-6)Galbeta1-4GlcNAcbeta1-3Galbeta1-4Glcbeta-Cer(d18:1/24:0); GalNAcalpha1-3Galbeta1-4GlcNAcbeta1-3(Galbeta1-4GlcNAcbeta1-6)Galbeta1-4GlcNAcbeta1-3Galbeta1-4Glcbeta-Cer(d18:1/24:1(15Z)); GalNAcalpha1-3Galbeta1-4GlcNAcbeta1-3(Galbeta1-4GlcNAcbeta1-6)Galbeta1-4GlcNAcbeta1-3Galbeta1-4Glcbeta-Cer(d18:1/26:0); GalNAcalpha1-3Galbeta1-4GlcNAcbeta1-3(Galbeta1-4GlcNAcbeta1-6)Galbeta1-4GlcNAcbeta1-3Galbeta1-4Glcbeta-Cer(d18:1/26:1(17Z)); GalNAcalpha1-3Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer; GalNAcalpha1-3Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/16:0); GalNAcalpha1-3Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/18:0); GalNAcalpha1-3Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/20:0); GalNAcalpha1-3Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/22:0); GalNAcalpha1-3Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/24:0); GalNAcalpha1-3Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/24:1(15Z)); GalNAcalpha1-3Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/26:0); GalNAcalpha1-3Galbeta1-4GlcNAcbeta1-3(GalNAcalpha1-3Galbeta1-4GlcNAcbeta1-6)Galbeta1-4GlcNAcbeta1-3Galbeta1-4Glcbeta-Cer(d18:1/26:1(17Z)); GalNAcalpha1-3GalNAcbeta1-3(Fucalpha1-2Galbeta1-3GalNAcbeta1-4)Galalpha1-4Galbeta1-4Glcbeta-Cer; GalNAcalpha1-3GalNAcbeta1-3(Fucalpha1-2Galbeta1-3GalNAcbeta1-4)Galalpha1-4Galbeta1-4Glcbeta-Cer(d18:1/16:0); GalNAcalpha1-3GalNAcbeta1-3(Fucalpha1-2Galbeta1-3GalNAcbeta1-4)Galalpha1-4Galbeta1-4Glcbeta-Cer(d18:1/18:0); GalNAcalpha1-3GalNAcbeta1-3(Fucalpha1-2Galbeta1-3GalNAcbeta1-4)Galalpha1-4Galbeta1-4Glcbeta-Cer(d18:1/20:0); GalNAcalpha1-3GalNAcbeta1-3(Fucalpha1-2Galbeta1-3GalNAcbeta1-4)Galalpha1-4Galbeta1-4Glcbeta-Cer(d18:1/22:0); GalNAcalpha1-3GalNAcbeta1-3(Fucalpha1-2Galbeta1-3GalNAcbeta1-4)Galalpha1-4Galbeta1-4Glcbeta-Cer(d18:1/24:0); GalNAcalpha1-3GalNAcbeta1-3(Fucalpha1-2Galbeta1-3GalNAcbeta1-4)Galalpha1-4Galbeta1-4Glcbeta-Cer(d18:1/24:1(15Z)); GalNAcalpha1-3GalNAcbeta1-3(Fucalpha1-2Galbeta1-3GalNAcbeta1-4)Galalpha1-4Galbeta1-4Glcbeta-Cer(d18:1/26:0); GalNAcalpha1-3GalNAcbeta1-3(Fucalpha1-2Galbeta1-3GalNAcbeta1-4)Galalpha1-4Galbeta1-4Glcbeta-Cer(d18:1/26:1(17Z)); GalNAcalpha1-3GalNAcbeta1-3(Galbeta1-3GalNAcbeta1-4)Galalpha1-4Galbeta1-4Glcbeta-Cer; GalNAcalpha1-3GalNAcbeta1-3(Galbeta1-3GalNAcbeta1-4)Galalpha1-4Galbeta1-4Glcbeta-Cer(d18:1/16:0); GalNAcalpha1-3GalNAcbeta1-3(Galbeta1-3GalNAcbeta1-4)Galalpha1-4Galbeta1-4Glcbeta-Cer(d18:1/18:0); GalNAcalpha1-3GalNAcbeta1-3(Galbeta1-3GalNAcbeta1-4)Galalpha1-4Galbeta1-4Glcbeta-Cer(d18:1/20:0); GalNAcalpha1-3GalNAcbeta1-3(Galbeta1-3GalNAcbeta1-4)Galalpha1-4Galbeta1-4Glcbeta-Cer(d18:1/22:0); GalNAcalpha1-3GalNAcbeta1-3(Galbeta1-3GalNAcbeta1-4)Galalpha1-4Galbeta1-4Glcbeta-Cer(d18:1/24:0); GalNAcalpha1-3GalNAcbeta1-3(Galbeta1-3GalNAcbeta1-4)Galalpha1-4Galbeta1-4Glcbeta-Cer(d18:1/24:1(15Z)); GalNAcalpha1-3GalNAcbeta1-3(Galbeta1-3GalNAcbeta1-4)Galalpha1-4Galbeta1-4Glcbeta-Cer(d18:1/26:0); GalNAcalpha1-3GalNAcbeta1-3(Galbeta1-3GalNAcbeta1-4)Galalpha1-4Galbeta1-4Glcbeta-Cer(d18:1/26:1(17Z)); GalNAcalpha1-3GalNAcbeta1-3(GalNAcbeta1-4)Galalpha1-4Galbeta1-4Glcbeta-Cer; GalNAcalpha1-3GalNAcbeta1-3(GalNAcbeta1-4)Galalpha1-4Galbeta1-4Glcbeta-Cer(d18:1/16:0); GalNAcalpha1-3GalNAcbeta1-3(GalNAcbeta1-4)Galalpha1-4Galbeta1-4Glcbeta-Cer(d18:1/18:0); GalNAcalpha1-3GalNAcbeta1-3(GalNAcbeta1-4)Galalpha1-4Galbeta1-4Glcbeta-Cer(d18:1/20:0); GalNAcalpha1-3GalNAcbeta1-3(GalNAcbeta1-4)Galalpha1-4Galbeta1-4Glcbeta-Cer(d18:1/22:0); GalNAcalpha1-3GalNAcbeta1-3(GalNAcbeta1-4)Galalpha1-4Galbeta1-4Glcbeta-Cer(d18:1/24:0); GalNAcalpha1-3GalNAcbeta1-3(GalNAcbeta1-4)Galalpha1-4Galbeta1-4Glcbeta-Cer(d18:1/24:1(15Z)); GalNAcalpha1-3GalNAcbeta1-3(GalNAcbeta1-4)Galalpha1-4Galbeta1-4Glcbeta-Cer(d18:1/26:0); GalNAcalpha1-3GalNAcbeta1-3(GalNAcbeta1-4)Galalpha1-4Galbeta1-4Glcbeta-Cer(d18:1/26:1(17Z)); GalNAcalpha1-3GalNAcbeta1-3Galalpha1-3(Galbeta1-4GlcNAcbeta1-6)Galbeta1-4Glcbeta-Cer; GalNAcalpha1-3GalNAcbeta1-3Galalpha1-3(Galbeta1-4GlcNAcbeta1-6)Galbeta1-4Glcbeta-Cer(d18:1/16:0); GalNAcalpha1-3GalNAcbeta1-3Galalpha1-3(Galbeta1-4GlcNAcbeta1-6)Galbeta1-4Glcbeta-Cer(d18:1/18:0); GalNAcalpha1-3GalNAcbeta1-3Galalpha1-3(Galbeta1-4GlcNAcbeta1-6)Galbeta1-4Glcbeta-Cer(d18:1/20:0); GalNAcalpha1-3GalNAcbeta1-3Galalpha1-3(Galbeta1-4GlcNAcbeta1-6)Galbeta1-4Glcbeta-Cer(d18:1/22:0); GalNAcalpha1-3GalNAcbeta1-3Galalpha1-3(Galbeta1-4GlcNAcbeta1-6)Galbeta1-4Glcbeta-Cer(d18:1/24:0); GalNAcalpha1-3GalNAcbeta1-3Galalpha1-3(Galbeta1-4GlcNAcbeta1-6)Galbeta1-4Glcbeta-Cer(d18:1/24:1(15Z)); GalNAcalpha1-3GalNAcbeta1-3Galalpha1-3(Galbeta1-4GlcNAcbeta1-6)Galbeta1-4Glcbeta-Cer(d18:1/26:0); GalNAcalpha1-3GalNAcbeta1-3Galalpha1-3(Galbeta1-4GlcNAcbeta1-6)Galbeta1-4Glcbeta-Cer(d18:1/26:1(17Z)); GalNAcalpha1-3GalNAcbeta1-3Galalpha1-3(GalNAcbeta1-4Galbeta1-4GlcNAcbeta1-6)Galbeta1-4Glcbeta-Cer; GalNAcalpha1-3GalNAcbeta1-3Galalpha1-3(GalNAcbeta1-4Galbeta1-4GlcNAcbeta1-6)Galbeta1-4Glcbeta-Cer(d18:1/16:0); GalNAcalpha1-3GalNAcbeta1-3Galalpha1-3(GalNAcbeta1-4Galbeta1-4GlcNAcbeta1-6)Galbeta1-4Glcbeta-Cer(d18:1/18:0); GalNAcalpha1-3GalNAcbeta1-3Galalpha1-3(GalNAcbeta1-4Galbeta1-4GlcNAcbeta1-6)Galbeta1-4Glcbeta-Cer(d18:1/20:0); GalNAcalpha1-3GalNAcbeta1-3Galalpha1-3(GalNAcbeta1-4Galbeta1-4GlcNAcbeta1-6)Galbeta1-4Glcbeta-Cer(d18:1/22:0); GalNAcalpha1-3GalNAcbeta1-3Galalpha1-3(GalNAcbeta1-4Galbeta1-4GlcNAcbeta1-6)Galbeta1-4Glcbeta-Cer(d18:1/24:0); GalNAcalpha1-3GalNAcbeta1-3Galalpha1-3(GalNAcbeta1-4Galbeta1-4GlcNAcbeta1-6)Galbeta1-4Glcbeta-Cer(d18:1/24:1(15Z)); GalNAcalpha1-3GalNAcbeta1-3Galalpha1-3(GalNAcbeta1-4Galbeta1-4GlcNAcbeta1-6)Galbeta1-4Glcbeta-Cer(d18:1/26:0); GalNAcalpha1-3GalNAcbeta1-3Galalpha1-3(GalNAcbeta1-4Galbeta1-4GlcNAcbeta1-6)Galbeta1-4Glcbeta-Cer(d18:1/26:1(17Z)); GalNAcalpha1-3GalNAcbeta1-3Galalpha1-3(GlcNAcbeta1-6)Galbeta1-4Glcbeta-Cer; GalNAcalpha1-3GalNAcbeta1-3Galalpha1-3(GlcNAcbeta1-6)Galbeta1-4Glcbeta-Cer(d18:1/16:0); GalNAcalpha1-3GalNAcbeta1-3Galalpha1-3(GlcNAcbeta1-6)Galbeta1-4Glcbeta-Cer(d18:1/18:0); GalNAcalpha1-3GalNAcbeta1-3Galalpha1-3(GlcNAcbeta1-6)Galbeta1-4Glcbeta-Cer(d18:1/20:0); GalNAcalpha1-3GalNAcbeta1-3Galalpha1-3(GlcNAcbeta1-6)Galbeta1-4Glcbeta-Cer(d18:1/22:0); GalNAcalpha1-3GalNAcbeta1-3Galalpha1-3(GlcNAcbeta1-6)Galbeta1-4Glcbeta-Cer(d18:1/24:0); GalNAcalpha1-3GalNAcbeta1-3Galalpha1-3(GlcNAcbeta1-6)Galbeta1-4Glcbeta-Cer(d18:1/24:1(15Z)); GalNAcalpha1-3GalNAcbeta1-3Galalpha1-3(GlcNAcbeta1-6)Galbeta1-4Glcbeta-Cer(d18:1/26:0); GalNAcalpha1-3GalNAcbeta1-3Galalpha1-3(GlcNAcbeta1-6)Galbeta1-4Glcbeta-Cer(d18:1/26:1(17Z)); GalNAcalpha1-3GalNAcbeta1-3Galalpha1-3(NeuAcalpha2-3(GalNAcbeta1-4)Galbeta1-4GlcNAcbeta1-6)Galbeta1-4Glcbeta-Cer; GalNAcalpha1-3GalNAcbeta1-3Galalpha1-3(NeuAcalpha2-3(GalNAcbeta1-4)Galbeta1-4GlcNAcbeta1-6)Galbeta1-4Glcbeta-Cer(d18:1/16:0); GalNAcalpha1-3GalNAcbeta1-3Galalpha1-3(NeuAcalpha2-3(GalNAcbeta1-4)Galbeta1-4GlcNAcbeta1-6)Galbeta1-4Glcbeta-Cer(d18:1/18:0); GalNAcalpha1-3GalNAcbeta1-3Galalpha1-3(NeuAcalpha2-3(GalNAcbeta1-4)Galbeta1-4GlcNAcbeta1-6)Galbeta1-4Glcbeta-Cer(d18:1/20:0); GalNAcalpha1-3GalNAcbeta1-3Galalpha1-3(NeuAcalpha2-3(GalNAcbeta1-4)Galbeta1-4GlcNAcbeta1-6)Galbeta1-4Glcbeta-Cer(d18:1/22:0); GalNAcalpha1-3GalNAcbeta1-3Galalpha1-3(NeuAcalpha2-3(GalNAcbeta1-4)Galbeta1-4GlcNAcbeta1-6)Galbeta1-4Glcbeta-Cer(d18:1/24:0); GalNAcalpha1-3GalNAcbeta1-3Galalpha1-3(NeuAcalpha2-3(GalNAcbeta1-4)Galbeta1-4GlcNAcbeta1-6)Galbeta1-4Glcbeta-Cer(d18:1/24:1(15Z)); GalNAcalpha1-3GalNAcbeta1-3Galalpha1-3(NeuAcalpha2-3(GalNAcbeta1-4)Galbeta1-4GlcNAcbeta1-6)Galbeta1-4Glcbeta-Cer(d18:1/26:0); GalNAcalpha1-3GalNAcbeta1-3Galalpha1-3(NeuAcalpha2-3(GalNAcbeta1-4)Galbeta1-4GlcNAcbeta1-6)Galbeta1-4Glcbeta-Cer(d18:1/26:1(17Z)); GalNAcalpha1-3GalNAcbeta1-3Galalpha1-3(NeuAcalpha2-3Galbeta1-4GlcNAcbeta1-6)Galbeta1-4Glcbeta-Cer; GalNAcalpha1-3GalNAcbeta1-3Galalpha1-3(NeuAcalpha2-3Galbeta1-4GlcNAcbeta1-6)Galbeta1-4Glcbeta-Cer(d18:1/16:0); GalNAcalpha1-3GalNAcbeta1-3Galalpha1-3(NeuAcalpha2-3Galbeta1-4GlcNAcbeta1-6)Galbeta1-4Glcbeta-Cer(d18:1/18:0); GalNAcalpha1-3GalNAcbeta1-3Galalpha1-3(NeuAcalpha2-3Galbeta1-4GlcNAcbeta1-6)Galbeta1-4Glcbeta-Cer(d18:1/20:0); GalNAcalpha1-3GalNAcbeta1-3Galalpha1-3(NeuAcalpha2-3Galbeta1-4GlcNAcbeta1-6)Galbeta1-4Glcbeta-Cer(d18:1/22:0); GalNAcalpha1-3GalNAcbeta1-3Galalpha1-3(NeuAcalpha2-3Galbeta1-4GlcNAcbeta1-6)Galbeta1-4Glcbeta-Cer(d18:1/24:0); GalNAcalpha1-3GalNAcbeta1-3Galalpha1-3(NeuAcalpha2-3Galbeta1-4GlcNAcbeta1-6)Galbeta1-4Glcbeta-Cer(d18:1/24:1(15Z)); GalNAcalpha1-3GalNAcbeta1-3Galalpha1-3(NeuAcalpha2-3Galbeta1-4GlcNAcbeta1-6)Galbeta1-4Glcbeta-Cer(d18:1/26:0); GalNAcalpha1-3GalNAcbeta1-3Galalpha1-3(NeuAcalpha2-3Galbeta1-4GlcNAcbeta1-6)Galbeta1-4Glcbeta-Cer(d18:1/26:1(17Z)); GalNAcalpha1-3GalNAcbeta1-3Galalpha1-3Galbeta1-4Glcbeta-Cer; GalNAcalpha1-3GalNAcbeta1-3Galalpha1-3Galbeta1-4Glcbeta-Cer(d18:1/16:0); GalNAcalpha1-3GalNAcbeta1-3Galalpha1-3Galbeta1-4Glcbeta-Cer(d18:1/18:0); GalNAcalpha1-3GalNAcbeta1-3Galalpha1-3Galbeta1-4Glcbeta-Cer(d18:1/20:0); GalNAcalpha1-3GalNAcbeta1-3Galalpha1-3Galbeta1-4Glcbeta-Cer(d18:1/22:0); GalNAcalpha1-3GalNAcbeta1-3Galalpha1-3Galbeta1-4Glcbeta-Cer(d18:1/24:0); GalNAcalpha1-3GalNAcbeta1-3Galalpha1-3Galbeta1-4Glcbeta-Cer(d18:1/24:1(15Z)); GalNAcalpha1-3GalNAcbeta1-3Galalpha1-3Galbeta1-4Glcbeta-Cer(d18:1/26:0); GalNAcalpha1-3GalNAcbeta1-3Galalpha1-3Galbeta1-4Glcbeta-Cer(d18:1/26:1(17Z)); GalNAcalpha1-3GalNAcbeta1-3Galalpha1-4Galbeta1-4Glcbeta-Cer; GalNAcalpha1-3GalNAcbeta1-3Galalpha1-4Galbeta1-4Glcbeta-Cer(d18:1/16:0); GalNAcalpha1-3GalNAcbeta1-3Galalpha1-4Galbeta1-4Glcbeta-Cer(d18:1/18:0); GalNAcalpha1-3GalNAcbeta1-3Galalpha1-4Galbeta1-4Glcbeta-Cer(d18:1/20:0); GalNAcalpha1-3GalNAcbeta1-3Galalpha1-4Galbeta1-4Glcbeta-Cer(d18:1/22:0); GalNAcalpha1-3GalNAcbeta1-3Galalpha1-4Galbeta1-4Glcbeta-Cer(d18:1/24:0); GalNAcalpha1-3GalNAcbeta1-3Galalpha1-4Galbeta1-4Glcbeta-Cer(d18:1/24:1(15Z)); GalNAcalpha1-3GalNAcbeta1-3Galalpha1-4Galbeta1-4Glcbeta-Cer(d18:1/26:0); GalNAcalpha1-3GalNAcbeta1-3Galalpha1-4Galbeta1-4Glcbeta-Cer(d18:1/26:1(17Z)); GalNAcalpha1-3GalNAcbeta1-3Galbeta1-3GalNAcbeta1-4(NeuAcalpha2-3)Galbeta1-4Glcbeta-Cer; GalNAcalpha1-3GalNAcbeta1-3Galbeta1-3GalNAcbeta1-4(NeuAcalpha2-3)Galbeta1-4Glcbeta-Cer(d18:1/16:0); GalNAcalpha1-3GalNAcbeta1-3Galbeta1-3GalNAcbeta1-4(NeuAcalpha2-3)Galbeta1-4Glcbeta-Cer(d18:1/18:0); GalNAcalpha1-3GalNAcbeta1-3Galbeta1-3GalNAcbeta1-4(NeuAcalpha2-3)Galbeta1-4Glcbeta-Cer(d18:1/20:0); GalNAcalpha1-3GalNAcbeta1-3Galbeta1-3GalNAcbeta1-4(NeuAcalpha2-3)Galbeta1-4Glcbeta-Cer(d18:1/22:0); GalNAcalpha1-3GalNAcbeta1-3Galbeta1-3GalNAcbeta1-4(NeuAcalpha2-3)Galbeta1-4Glcbeta-Cer(d18:1/24:0); GalNAcalpha1-3GalNAcbeta1-3Galbeta1-3GalNAcbeta1-4(NeuAcalpha2-3)Galbeta1-4Glcbeta-Cer(d18:1/24:1(15Z)); GalNAcalpha1-3GalNAcbeta1-3Galbeta1-3GalNAcbeta1-4(NeuAcalpha2-3)Galbeta1-4Glcbeta-Cer(d18:1/26:0); GalNAcalpha1-3GalNAcbeta1-3Galbeta1-3GalNAcbeta1-4(NeuAcalpha2-3)Galbeta1-4Glcbeta-Cer(d18:1/26:1(17Z)); GalNAcb1-4(Fuca1-3)GlcNAcb1-3Manb1-4Glcb-Cer; GalNAcb1-4(GlcNAca1-2Fuca1-3)GlcNAcb1-3Manb1-4Glcb-Cer; GalNAcbeta1-3(Fucalpha1-2)Galbeta1-4GlcNAcbeta1-3Galbeta1-4Glcbeta-Cer; GalNAcbeta1-3(Fucalpha1-2)Galbeta1-4GlcNAcbeta1-3Galbeta1-4Glcbeta-Cer(d18:1/16:0); GalNAcbeta1-3(Fucalpha1-2)Galbeta1-4GlcNAcbeta1-3Galbeta1-4Glcbeta-Cer(d18:1/18:0); GalNAcbeta1-3(Fucalpha1-2)Galbeta1-4GlcNAcbeta1-3Galbeta1-4Glcbeta-Cer(d18:1/20:0); GalNAcbeta1-3(Fucalpha1-2)Galbeta1-4GlcNAcbeta1-3Galbeta1-4Glcbeta-Cer(d18:1/22:0); GalNAcbeta1-3(Fucalpha1-2)Galbeta1-4GlcNAcbeta1-3Galbeta1-4Glcbeta-Cer(d18:1/24:0); GalNAcbeta1-3(Fucalpha1-2)Galbeta1-4GlcNAcbeta1-3Galbeta1-4Glcbeta-Cer(d18:1/24:1(15Z)); GalNAcbeta1-3(Fucalpha1-2)Galbeta1-4GlcNAcbeta1-3Galbeta1-4Glcbeta-Cer(d18:1/26:0); GalNAcbeta1-3(Fucalpha1-2)Galbeta1-4GlcNAcbeta1-3Galbeta1-4Glcbeta-Cer(d18:1/26:1(17Z)); GalNAcbeta1-3(Fucalpha2-3Galbeta1-3GalNAcbeta1-4)Galalpha1-4Galbeta1-4Glcbeta-Cer; GalNAcbeta1-3(Fucalpha2-3Galbeta1-3GalNAcbeta1-4)Galalpha1-4Galbeta1-4Glcbeta-Cer(d18:1/16:0); GalNAcbeta1-3(Fucalpha2-3Galbeta1-3GalNAcbeta1-4)Galalpha1-4Galbeta1-4Glcbeta-Cer(d18:1/18:0); GalNAcbeta1-3(Fucalpha2-3Galbeta1-3GalNAcbeta1-4)Galalpha1-4Galbeta1-4Glcbeta-Cer(d18:1/20:0); GalNAcbeta1-3(Fucalpha2-3Galbeta1-3GalNAcbeta1-4)Galalpha1-4Galbeta1-4Glcbeta-Cer(d18:1/22:0); GalNAcbeta1-3(Fucalpha2-3Galbeta1-3GalNAcbeta1-4)Galalpha1-4Galbeta1-4Glcbeta-Cer(d18:1/24:0); GalNAcbeta1-3(Fucalpha2-3Galbeta1-3GalNAcbeta1-4)Galalpha1-4Galbeta1-4Glcbeta-Cer(d18:1/24:1(15Z)); GalNAcbeta1-3(Fucalpha2-3Galbeta1-3GalNAcbeta1-4)Galalpha1-4Galbeta1-4Glcbeta-Cer(d18:1/26:0); GalNAcbeta1-3(Fucalpha2-3Galbeta1-3GalNAcbeta1-4)Galalpha1-4Galbeta1-4Glcbeta-Cer(d18:1/26:1(17Z)); GalNAcbeta1-3(Galbeta1-3GalNAcbeta1-4)Galalpha1-4Galbeta1-4Glcbeta-Cer; GalNAcbeta1-3(Galbeta1-3GalNAcbeta1-4)Galalpha1-4Galbeta1-4Glcbeta-Cer(d18:1/16:0); GalNAcbeta1-3(Galbeta1-3GalNAcbeta1-4)Galalpha1-4Galbeta1-4Glcbeta-Cer(d18:1/18:0); GalNAcbeta1-3(Galbeta1-3GalNAcbeta1-4)Galalpha1-4Galbeta1-4Glcbeta-Cer(d18:1/20:0); GalNAcbeta1-3(Galbeta1-3GalNAcbeta1-4)Galalpha1-4Galbeta1-4Glcbeta-Cer(d18:1/22:0); GalNAcbeta1-3(Galbeta1-3GalNAcbeta1-4)Galalpha1-4Galbeta1-4Glcbeta-Cer(d18:1/24:0); GalNAcbeta1-3(Galbeta1-3GalNAcbeta1-4)Galalpha1-4Galbeta1-4Glcbeta-Cer(d18:1/24:1(15Z)); GalNAcbeta1-3(Galbeta1-3GalNAcbeta1-4)Galalpha1-4Galbeta1-4Glcbeta-Cer(d18:1/26:0); GalNAcbeta1-3(Galbeta1-3GalNAcbeta1-4)Galalpha1-4Galbeta1-4Glcbeta-Cer(d18:1/26:1(17Z)); GalNAcbeta1-3(GalNAcbeta1-4)Galalpha1-4Galbeta1-4Glcbeta-Cer; GalNAcbeta1-3(GalNAcbeta1-4)Galalpha1-4Galbeta1-4Glcbeta-Cer(d18:1/16:0); GalNAcbeta1-3(GalNAcbeta1-4)Galalpha1-4Galbeta1-4Glcbeta-Cer(d18:1/18:0); GalNAcbeta1-3(GalNAcbeta1-4)Galalpha1-4Galbeta1-4Glcbeta-Cer(d18:1/20:0); GalNAcbeta1-3(GalNAcbeta1-4)Galalpha1-4Galbeta1-4Glcbeta-Cer(d18:1/22:0); GalNAcbeta1-3(GalNAcbeta1-4)Galalpha1-4Galbeta1-4Glcbeta-Cer(d18:1/24:0); GalNAcbeta1-3(GalNAcbeta1-4)Galalpha1-4Galbeta1-4Glcbeta-Cer(d18:1/24:1(15Z)); GalNAcbeta1-3(GalNAcbeta1-4)Galalpha1-4Galbeta1-4Glcbeta-Cer(d18:1/26:0); GalNAcbeta1-3(GalNAcbeta1-4)Galalpha1-4Galbeta1-4Glcbeta-Cer(d18:1/26:1(17Z)); GalNAcbeta1-3Galalpha1-3(Fucalpha1-2)Galbeta1-3GalNAcbeta1-4Galbeta1-4Glcbeta-Cer; GalNAcbeta1-3Galalpha1-3(Fucalpha1-2)Galbeta1-3GalNAcbeta1-4Galbeta1-4Glcbeta-Cer(d18:1/16:0); GalNAcbeta1-3Galalpha1-3(Fucalpha1-2)Galbeta1-3GalNAcbeta1-4Galbeta1-4Glcbeta-Cer(d18:1/18:0); GalNAcbeta1-3Galalpha1-3(Fucalpha1-2)Galbeta1-3GalNAcbeta1-4Galbeta1-4Glcbeta-Cer(d18:1/20:0); GalNAcbeta1-3Galalpha1-3(Fucalpha1-2)Galbeta1-3GalNAcbeta1-4Galbeta1-4Glcbeta-Cer(d18:1/22:0); GalNAcbeta1-3Galalpha1-3(Fucalpha1-2)Galbeta1-3GalNAcbeta1-4Galbeta1-4Glcbeta-Cer(d18:1/24:0); GalNAcbeta1-3Galalpha1-3(Fucalpha1-2)Galbeta1-3GalNAcbeta1-4Galbeta1-4Glcbeta-Cer(d18:1/24:1(15Z)); GalNAcbeta1-3Galalpha1-3(Fucalpha1-2)Galbeta1-3GalNAcbeta1-4Galbeta1-4Glcbeta-Cer(d18:1/26:0); GalNAcbeta1-3Galalpha1-3(Fucalpha1-2)Galbeta1-3GalNAcbeta1-4Galbeta1-4Glcbeta-Cer(d18:1/26:1(17Z)); GalNAcbeta1-3Galalpha1-3(Galbeta1-4GlcNAcbeta1-6)Galbeta1-4Glcbeta-Cer; GalNAcbeta1-3Galalpha1-3(Galbeta1-4GlcNAcbeta1-6)Galbeta1-4Glcbeta-Cer(d18:1/16:0); GalNAcbeta1-3Galalpha1-3(Galbeta1-4GlcNAcbeta1-6)Galbeta1-4Glcbeta-Cer(d18:1/18:0); GalNAcbeta1-3Galalpha1-3(Galbeta1-4GlcNAcbeta1-6)Galbeta1-4Glcbeta-Cer(d18:1/20:0); GalNAcbeta1-3Galalpha1-3(Galbeta1-4GlcNAcbeta1-6)Galbeta1-4Glcbeta-Cer(d18:1/22:0); GalNAcbeta1-3Galalpha1-3(Galbeta1-4GlcNAcbeta1-6)Galbeta1-4Glcbeta-Cer(d18:1/24:0); GalNAcbeta1-3Galalpha1-3(Galbeta1-4GlcNAcbeta1-6)Galbeta1-4Glcbeta-Cer(d18:1/24:1(15Z)); GalNAcbeta1-3Galalpha1-3(Galbeta1-4GlcNAcbeta1-6)Galbeta1-4Glcbeta-Cer(d18:1/26:0); GalNAcbeta1-3Galalpha1-3(Galbeta1-4GlcNAcbeta1-6)Galbeta1-4Glcbeta-Cer(d18:1/26:1(17Z)); GalNAcbeta1-3Galalpha1-3(GalNAcbeta1-4Galbeta1-4GlcNAcbeta1-6)Galbeta1-4Glcbeta-Cer; GalNAcbeta1-3Galalpha1-3(GalNAcbeta1-4Galbeta1-4GlcNAcbeta1-6)Galbeta1-4Glcbeta-Cer(d18:1/16:0); GalNAcbeta1-3Galalpha1-3(GalNAcbeta1-4Galbeta1-4GlcNAcbeta1-6)Galbeta1-4Glcbeta-Cer(d18:1/18:0); GalNAcbeta1-3Galalpha1-3(GalNAcbeta1-4Galbeta1-4GlcNAcbeta1-6)Galbeta1-4Glcbeta-Cer(d18:1/20:0); GalNAcbeta1-3Galalpha1-3(GalNAcbeta1-4Galbeta1-4GlcNAcbeta1-6)Galbeta1-4Glcbeta-Cer(d18:1/22:0); GalNAcbeta1-3Galalpha1-3(GalNAcbeta1-4Galbeta1-4GlcNAcbeta1-6)Galbeta1-4Glcbeta-Cer(d18:1/24:0); GalNAcbeta1-3Galalpha1-3(GalNAcbeta1-4Galbeta1-4GlcNAcbeta1-6)Galbeta1-4Glcbeta-Cer(d18:1/24:1(15Z)); GalNAcbeta1-3Galalpha1-3(GalNAcbeta1-4Galbeta1-4GlcNAcbeta1-6)Galbeta1-4Glcbeta-Cer(d18:1/26:0); GalNAcbeta1-3Galalpha1-3(GalNAcbeta1-4Galbeta1-4GlcNAcbeta1-6)Galbeta1-4Glcbeta-Cer(d18:1/26:1(17Z)); GalNAcbeta1-3Galalpha1-3(GlcNAcbeta1-6)Galbeta1-4Glcbeta-Cer; GalNAcbeta1-3Galalpha1-3(GlcNAcbeta1-6)Galbeta1-4Glcbeta-Cer(d18:1/16:0); GalNAcbeta1-3Galalpha1-3(GlcNAcbeta1-6)Galbeta1-4Glcbeta-Cer(d18:1/18:0); GalNAcbeta1-3Galalpha1-3(GlcNAcbeta1-6)Galbeta1-4Glcbeta-Cer(d18:1/20:0); GalNAcbeta1-3Galalpha1-3(GlcNAcbeta1-6)Galbeta1-4Glcbeta-Cer(d18:1/22:0); GalNAcbeta1-3Galalpha1-3(GlcNAcbeta1-6)Galbeta1-4Glcbeta-Cer(d18:1/24:0); GalNAcbeta1-3Galalpha1-3(GlcNAcbeta1-6)Galbeta1-4Glcbeta-Cer(d18:1/24:1(15Z)); GalNAcbeta1-3Galalpha1-3(GlcNAcbeta1-6)Galbeta1-4Glcbeta-Cer(d18:1/26:0); GalNAcbeta1-3Galalpha1-3(GlcNAcbeta1-6)Galbeta1-4Glcbeta-Cer(d18:1/26:1(17Z)); GalNAcbeta1-3Galalpha1-3(NeuAcalpha2-3Galbeta1-4GlcNAcbeta1-6)Galbeta1-4Glcbeta-Cer; GalNAcbeta1-3Galalpha1-3(NeuAcalpha2-3Galbeta1-4GlcNAcbeta1-6)Galbeta1-4Glcbeta-Cer(d18:1/16:0); GalNAcbeta1-3Galalpha1-3(NeuAcalpha2-3Galbeta1-4GlcNAcbeta1-6)Galbeta1-4Glcbeta-Cer(d18:1/18:0); GalNAcbeta1-3Galalpha1-3(NeuAcalpha2-3Galbeta1-4GlcNAcbeta1-6)Galbeta1-4Glcbeta-Cer(d18:1/20:0); GalNAcbeta1-3Galalpha1-3(NeuAcalpha2-3Galbeta1-4GlcNAcbeta1-6)Galbeta1-4Glcbeta-Cer(d18:1/22:0); GalNAcbeta1-3Galalpha1-3(NeuAcalpha2-3Galbeta1-4GlcNAcbeta1-6)Galbeta1-4Glcbeta-Cer(d18:1/24:0); GalNAcbeta1-3Galalpha1-3(NeuAcalpha2-3Galbeta1-4GlcNAcbeta1-6)Galbeta1-4Glcbeta-Cer(d18:1/24:1(15Z)); GalNAcbeta1-3Galalpha1-3(NeuAcalpha2-3Galbeta1-4GlcNAcbeta1-6)Galbeta1-4Glcbeta-Cer(d18:1/26:0); GalNAcbeta1-3Galalpha1-3(NeuAcalpha2-3Galbeta1-4GlcNAcbeta1-6)Galbeta1-4Glcbeta-Cer(d18:1/26:1(17Z)); GalNAcbeta1-3Galalpha1-3Galalpha1-3Galalpha1-3Galalpha1-3Galalpha1-3Galalpha1-4Galbeta1-4Glcbeta-Cer; GalNAcbeta1-3Galalpha1-3Galalpha1-3Galalpha1-3Galalpha1-3Galalpha1-3Galalpha1-4Galbeta1-4Glcbeta-Cer(d18:1/16:0); GalNAcbeta1-3Galalpha1-3Galalpha1-3Galalpha1-3Galalpha1-3Galalpha1-3Galalpha1-4Galbeta1-4Glcbeta-Cer(d18:1/18:0); GalNAcbeta1-3Galalpha1-3Galalpha1-3Galalpha1-3Galalpha1-3Galalpha1-3Galalpha1-4Galbeta1-4Glcbeta-Cer(d18:1/20:0); GalNAcbeta1-3Galalpha1-3Galalpha1-3Galalpha1-3Galalpha1-3Galalpha1-3Galalpha1-4Galbeta1-4Glcbeta-Cer(d18:1/22:0); GalNAcbeta1-3Galalpha1-3Galalpha1-3Galalpha1-3Galalpha1-3Galalpha1-3Galalpha1-4Galbeta1-4Glcbeta-Cer(d18:1/24:0); GalNAcbeta1-3Galalpha1-3Galalpha1-3Galalpha1-3Galalpha1-3Galalpha1-3Galalpha1-4Galbeta1-4Glcbeta-Cer(d18:1/24:1(15Z)); GalNAcbeta1-3Galalpha1-3Galalpha1-3Galalpha1-3Galalpha1-3Galalpha1-3Galalpha1-4Galbeta1-4Glcbeta-Cer(d18:1/26:0); GalNAcbeta1-3Galalpha1-3Galalpha1-3Galalpha1-3Galalpha1-3Galalpha1-3Galalpha1-4Galbeta1-4Glcbeta-Cer(d18:1/26:1(17Z)); GalNAcbeta1-3Galalpha1-3Galalpha1-3Galalpha1-3Galalpha1-3Galalpha1-4Galbeta1-4Glcbeta-Cer; GalNAcbeta1-3Galalpha1-3Galalpha1-3Galalpha1-3Galalpha1-3Galalpha1-4Galbeta1-4Glcbeta-Cer(d18:1/16:0); GalNAcbeta1-3Galalpha1-3Galalpha1-3Galalpha1-3Galalpha1-3Galalpha1-4Galbeta1-4Glcbeta-Cer(d18:1/18:0); GalNAcbeta1-3Galalpha1-3Galalpha1-3Galalpha1-3Galalpha1-3Galalpha1-4Galbeta1-4Glcbeta-Cer(d18:1/20:0); GalNAcbeta1-3Galalpha1-3Galalpha1-3Galalpha1-3Galalpha1-3Galalpha1-4Galbeta1-4Glcbeta-Cer(d18:1/22:0); GalNAcbeta1-3Galalpha1-3Galalpha1-3Galalpha1-3Galalpha1-3Galalpha1-4Galbeta1-4Glcbeta-Cer(d18:1/24:0); GalNAcbeta1-3Galalpha1-3Galalpha1-3Galalpha1-3Galalpha1-3Galalpha1-4Galbeta1-4Glcbeta-Cer(d18:1/24:1(15Z)); GalNAcbeta1-3Galalpha1-3Galalpha1-3Galalpha1-3Galalpha1-3Galalpha1-4Galbeta1-4Glcbeta-Cer(d18:1/26:0); GalNAcbeta1-3Galalpha1-3Galalpha1-3Galalpha1-3Galalpha1-3Galalpha1-4Galbeta1-4Glcbeta-Cer(d18:1/26:1(17Z)); GalNAcbeta1-3Galalpha1-3Galalpha1-3Galalpha1-3Galalpha1-4Galbeta1-4Glcbeta-Cer; GalNAcbeta1-3Galalpha1-3Galalpha1-3Galalpha1-3Galalpha1-4Galbeta1-4Glcbeta-Cer(d18:1/16:0); GalNAcbeta1-3Galalpha1-3Galalpha1-3Galalpha1-3Galalpha1-4Galbeta1-4Glcbeta-Cer(d18:1/18:0); GalNAcbeta1-3Galalpha1-3Galalpha1-3Galalpha1-3Galalpha1-4Galbeta1-4Glcbeta-Cer(d18:1/20:0); GalNAcbeta1-3Galalpha1-3Galalpha1-3Galalpha1-3Galalpha1-4Galbeta1-4Glcbeta-Cer(d18:1/22:0); GalNAcbeta1-3Galalpha1-3Galalpha1-3Galalpha1-3Galalpha1-4Galbeta1-4Glcbeta-Cer(d18:1/24:0); GalNAcbeta1-3Galalpha1-3Galalpha1-3Galalpha1-3Galalpha1-4Galbeta1-4Glcbeta-Cer(d18:1/24:1(15Z)); GalNAcbeta1-3Galalpha1-3Galalpha1-3Galalpha1-3Galalpha1-4Galbeta1-4Glcbeta-Cer(d18:1/26:0); GalNAcbeta1-3Galalpha1-3Galalpha1-3Galalpha1-3Galalpha1-4Galbeta1-4Glcbeta-Cer(d18:1/26:1(17Z)); GalNAcbeta1-3Galalpha1-3Galalpha1-3Galalpha1-4Galbeta1-4Glcbeta-Cer; GalNAcbeta1-3Galalpha1-3Galalpha1-3Galalpha1-4Galbeta1-4Glcbeta-Cer(d18:1/16:0); GalNAcbeta1-3Galalpha1-3Galalpha1-3Galalpha1-4Galbeta1-4Glcbeta-Cer(d18:1/18:0); GalNAcbeta1-3Galalpha1-3Galalpha1-3Galalpha1-4Galbeta1-4Glcbeta-Cer(d18:1/20:0); GalNAcbeta1-3Galalpha1-3Galalpha1-3Galalpha1-4Galbeta1-4Glcbeta-Cer(d18:1/22:0); GalNAcbeta1-3Galalpha1-3Galalpha1-3Galalpha1-4Galbeta1-4Glcbeta-Cer(d18:1/24:0); GalNAcbeta1-3Galalpha1-3Galalpha1-3Galalpha1-4Galbeta1-4Glcbeta-Cer(d18:1/24:1(15Z)); GalNAcbeta1-3Galalpha1-3Galalpha1-3Galalpha1-4Galbeta1-4Glcbeta-Cer(d18:1/26:0); GalNAcbeta1-3Galalpha1-3Galalpha1-3Galalpha1-4Galbeta1-4Glcbeta-Cer(d18:1/26:1(17Z)); GalNAcbeta1-3Galalpha1-3Galalpha1-3Galbeta1-4Glcbeta-Cer; GalNAcbeta1-3Galalpha1-3Galalpha1-3Galbeta1-4Glcbeta-Cer(d18:1/16:0); GalNAcbeta1-3Galalpha1-3Galalpha1-3Galbeta1-4Glcbeta-Cer(d18:1/18:0); GalNAcbeta1-3Galalpha1-3Galalpha1-3Galbeta1-4Glcbeta-Cer(d18:1/20:0); GalNAcbeta1-3Galalpha1-3Galalpha1-3Galbeta1-4Glcbeta-Cer(d18:1/22:0); GalNAcbeta1-3Galalpha1-3Galalpha1-3Galbeta1-4Glcbeta-Cer(d18:1/24:0); GalNAcbeta1-3Galalpha1-3Galalpha1-3Galbeta1-4Glcbeta-Cer(d18:1/24:1(15Z)); GalNAcbeta1-3Galalpha1-3Galalpha1-3Galbeta1-4Glcbeta-Cer(d18:1/26:0); GalNAcbeta1-3Galalpha1-3Galalpha1-3Galbeta1-4Glcbeta-Cer(d18:1/26:1(17Z)); GalNAcbeta1-3Galalpha1-3Galalpha1-4Galbeta1-4Glcbeta-Cer; GalNAcbeta1-3Galalpha1-3Galalpha1-4Galbeta1-4Glcbeta-Cer(d18:1/16:0); GalNAcbeta1-3Galalpha1-3Galalpha1-4Galbeta1-4Glcbeta-Cer(d18:1/18:0); GalNAcbeta1-3Galalpha1-3Galalpha1-4Galbeta1-4Glcbeta-Cer(d18:1/20:0); GalNAcbeta1-3Galalpha1-3Galalpha1-4Galbeta1-4Glcbeta-Cer(d18:1/22:0); GalNAcbeta1-3Galalpha1-3Galalpha1-4Galbeta1-4Glcbeta-Cer(d18:1/24:0); GalNAcbeta1-3Galalpha1-3Galalpha1-4Galbeta1-4Glcbeta-Cer(d18:1/24:1(15Z)); GalNAcbeta1-3Galalpha1-3Galalpha1-4Galbeta1-4Glcbeta-Cer(d18:1/26:0); GalNAcbeta1-3Galalpha1-3Galalpha1-4Galbeta1-4Glcbeta-Cer(d18:1/26:1(17Z)); GalNAcbeta1-3Galalpha1-3Galbeta1-3GalNAcbeta1-4Galbeta1-4Glcbeta-Cer; GalNAcbeta1-3Galalpha1-3Galbeta1-3GalNAcbeta1-4Galbeta1-4Glcbeta-Cer(d18:1/16:0); GalNAcbeta1-3Galalpha1-3Galbeta1-3GalNAcbeta1-4Galbeta1-4Glcbeta-Cer(d18:1/18:0); GalNAcbeta1-3Galalpha1-3Galbeta1-3GalNAcbeta1-4Galbeta1-4Glcbeta-Cer(d18:1/20:0); GalNAcbeta1-3Galalpha1-3Galbeta1-3GalNAcbeta1-4Galbeta1-4Glcbeta-Cer(d18:1/22:0); GalNAcbeta1-3Galalpha1-3Galbeta1-3GalNAcbeta1-4Galbeta1-4Glcbeta-Cer(d18:1/24:0); GalNAcbeta1-3Galalpha1-3Galbeta1-3GalNAcbeta1-4Galbeta1-4Glcbeta-Cer(d18:1/24:1(15Z)); GalNAcbeta1-3Galalpha1-3Galbeta1-3GalNAcbeta1-4Galbeta1-4Glcbeta-Cer(d18:1/26:0); GalNAcbeta1-3Galalpha1-3Galbeta1-3GalNAcbeta1-4Galbeta1-4Glcbeta-Cer(d18:1/26:1(17Z)); GalNAcbeta1-3Galalpha1-3Galbeta1-4Glcbeta-Cer; GalNAcbeta1-3Galalpha1-3Galbeta1-4Glcbeta-Cer; GalNAcbeta1-3Galalpha1-3Galbeta1-4Glcbeta-Cer(d18:1/16:0); GalNAcbeta1-3Galalpha1-3Galbeta1-4Glcbeta-Cer(d18:1/18:0); GalNAcbeta1-3Galalpha1-3Galbeta1-4Glcbeta-Cer(d18:1/20:0); GalNAcbeta1-3Galalpha1-3Galbeta1-4Glcbeta-Cer(d18:1/22:0); GalNAcbeta1-3Galalpha1-3Galbeta1-4Glcbeta-Cer(d18:1/24:0); GalNAcbeta1-3Galalpha1-3Galbeta1-4Glcbeta-Cer(d18:1/24:1(15Z)); GalNAcbeta1-3Galalpha1-3Galbeta1-4Glcbeta-Cer(d18:1/26:0); GalNAcbeta1-3Galalpha1-3Galbeta1-4Glcbeta-Cer(d18:1/26:1(17Z)); GalNAcbeta1-3Galalpha1-3Galbeta1-4GlcNAcbeta1-3Galbeta1-4Glcbeta-Cer; GalNAcbeta1-3Galalpha1-3Galbeta1-4GlcNAcbeta1-3Galbeta1-4Glcbeta-Cer(d18:1/16:0); GalNAcbeta1-3Galalpha1-3Galbeta1-4GlcNAcbeta1-3Galbeta1-4Glcbeta-Cer(d18:1/18:0); GalNAcbeta1-3Galalpha1-3Galbeta1-4GlcNAcbeta1-3Galbeta1-4Glcbeta-Cer(d18:1/20:0); GalNAcbeta1-3Galalpha1-3Galbeta1-4GlcNAcbeta1-3Galbeta1-4Glcbeta-Cer(d18:1/22:0); GalNAcbeta1-3Galalpha1-3Galbeta1-4GlcNAcbeta1-3Galbeta1-4Glcbeta-Cer(d18:1/24:0); GalNAcbeta1-3Galalpha1-3Galbeta1-4GlcNAcbeta1-3Galbeta1-4Glcbeta-Cer(d18:1/24:1(15Z)); GalNAcbeta1-3Galalpha1-3Galbeta1-4GlcNAcbeta1-3Galbeta1-4Glcbeta-Cer(d18:1/26:0); GalNAcbeta1-3Galalpha1-3Galbeta1-4GlcNAcbeta1-3Galbeta1-4Glcbeta-Cer(d18:1/26:1(17Z)); GalNAcbeta1-3Galalpha1-4Galbeta1-4Glcbeta-Cer; GalNAcbeta1-3Galalpha1-4Galbeta1-4Glcbeta-Cer(d18:1/16:0); GalNAcbeta1-3Galalpha1-4Galbeta1-4Glcbeta-Cer(d18:1/18:0); GalNAcbeta1-3Galalpha1-4Galbeta1-4Glcbeta-Cer(d18:1/20:0); GalNAcbeta1-3Galalpha1-4Galbeta1-4Glcbeta-Cer(d18:1/22:0); GalNAcbeta1-3Galalpha1-4Galbeta1-4Glcbeta-Cer(d18:1/24:0); GalNAcbeta1-3Galalpha1-4Galbeta1-4Glcbeta-Cer(d18:1/24:1(15Z)); GalNAcbeta1-3Galalpha1-4Galbeta1-4Glcbeta-Cer(d18:1/26:0); GalNAcbeta1-3Galalpha1-4Galbeta1-4Glcbeta-Cer(d18:1/26:1(17Z)); GalNAcbeta1-3Galbeta1-3GalNAcbeta1-4(NeuAcalpha2-3)Galbeta1-4Glcbeta-Cer; GalNAcbeta1-3Galbeta1-3GalNAcbeta1-4(NeuAcalpha2-3)Galbeta1-4Glcbeta-Cer(d18:1/16:0); GalNAcbeta1-3Galbeta1-3GalNAcbeta1-4(NeuAcalpha2-3)Galbeta1-4Glcbeta-Cer(d18:1/18:0); GalNAcbeta1-3Galbeta1-3GalNAcbeta1-4(NeuAcalpha2-3)Galbeta1-4Glcbeta-Cer(d18:1/20:0); GalNAcbeta1-3Galbeta1-3GalNAcbeta1-4(NeuAcalpha2-3)Galbeta1-4Glcbeta-Cer(d18:1/22:0); GalNAcbeta1-3Galbeta1-3GalNAcbeta1-4(NeuAcalpha2-3)Galbeta1-4Glcbeta-Cer(d18:1/24:0); GalNAcbeta1-3Galbeta1-3GalNAcbeta1-4(NeuAcalpha2-3)Galbeta1-4Glcbeta-Cer(d18:1/24:1(15Z)); GalNAcbeta1-3Galbeta1-3GalNAcbeta1-4(NeuAcalpha2-3)Galbeta1-4Glcbeta-Cer(d18:1/26:0); GalNAcbeta1-3Galbeta1-3GalNAcbeta1-4(NeuAcalpha2-3)Galbeta1-4Glcbeta-Cer(d18:1/26:1(17Z)); GalNAcbeta1-3Galbeta1-3GalNAcbeta1-4Galbeta1-4Glcbeta-Cer; GalNAcbeta1-3Galbeta1-3GalNAcbeta1-4Galbeta1-4Glcbeta-Cer(d18:1/16:0); GalNAcbeta1-3Galbeta1-3GalNAcbeta1-4Galbeta1-4Glcbeta-Cer(d18:1/18:0); GalNAcbeta1-3Galbeta1-3GalNAcbeta1-4Galbeta1-4Glcbeta-Cer(d18:1/20:0); GalNAcbeta1-3Galbeta1-3GalNAcbeta1-4Galbeta1-4Glcbeta-Cer(d18:1/22:0); GalNAcbeta1-3Galbeta1-3GalNAcbeta1-4Galbeta1-4Glcbeta-Cer(d18:1/24:0); GalNAcbeta1-3Galbeta1-3GalNAcbeta1-4Galbeta1-4Glcbeta-Cer(d18:1/24:1(15Z)); GalNAcbeta1-3Galbeta1-3GalNAcbeta1-4Galbeta1-4Glcbeta-Cer(d18:1/26:0); GalNAcbeta1-3Galbeta1-3GalNAcbeta1-4Galbeta1-4Glcbeta-Cer(d18:1/26:1(17Z)); GalNAcbeta1-3Galbeta1-4GlcNAcbeta1-3Galbeta1-4Glcbeta-Cer; GalNAcbeta1-3Galbeta1-4GlcNAcbeta1-3Galbeta1-4Glcbeta-Cer(d18:1/16:0); GalNAcbeta1-3Galbeta1-4GlcNAcbeta1-3Galbeta1-4Glcbeta-Cer(d18:1/18:0); GalNAcbeta1-3Galbeta1-4GlcNAcbeta1-3Galbeta1-4Glcbeta-Cer(d18:1/20:0); GalNAcbeta1-3Galbeta1-4GlcNAcbeta1-3Galbeta1-4Glcbeta-Cer(d18:1/22:0); GalNAcbeta1-3Galbeta1-4GlcNAcbeta1-3Galbeta1-4Glcbeta-Cer(d18:1/24:0); GalNAcbeta1-3Galbeta1-4GlcNAcbeta1-3Galbeta1-4Glcbeta-Cer(d18:1/24:1(15Z)); GalNAcbeta1-3Galbeta1-4GlcNAcbeta1-3Galbeta1-4Glcbeta-Cer(d18:1/26:0); GalNAcbeta1-3Galbeta1-4GlcNAcbeta1-3Galbeta1-4Glcbeta-Cer(d18:1/26:1(17Z)); GalNAcbeta1-3GalNAcbeta1-3Galalpha1-4Galbeta1-4Glcbeta-Cer; GalNAcbeta1-3GalNAcbeta1-3Galalpha1-4Galbeta1-4Glcbeta-Cer(d18:1/16:0); GalNAcbeta1-3GalNAcbeta1-3Galalpha1-4Galbeta1-4Glcbeta-Cer(d18:1/18:0); GalNAcbeta1-3GalNAcbeta1-3Galalpha1-4Galbeta1-4Glcbeta-Cer(d18:1/20:0); GalNAcbeta1-3GalNAcbeta1-3Galalpha1-4Galbeta1-4Glcbeta-Cer(d18:1/22:0); GalNAcbeta1-3GalNAcbeta1-3Galalpha1-4Galbeta1-4Glcbeta-Cer(d18:1/24:0); GalNAcbeta1-3GalNAcbeta1-3Galalpha1-4Galbeta1-4Glcbeta-Cer(d18:1/24:1(15Z)); GalNAcbeta1-3GalNAcbeta1-3Galalpha1-4Galbeta1-4Glcbeta-Cer(d18:1/26:0); GalNAcbeta1-3GalNAcbeta1-3Galalpha1-4Galbeta1-4Glcbeta-Cer(d18:1/26:1(17Z)); GalNAcbeta1-4(9-OAcNeuAcalpha2-8NeuAcalpha2-8NeuAcalpha2-3)Galbeta1-4Glcbeta-Cer; GalNAcbeta1-4(Galalpha1-3Galbeta1-4GlcNAcbeta1-3)Galbeta1-4Glcbeta-Cer; GalNAcbeta1-4(Galalpha1-3Galbeta1-4GlcNAcbeta1-3)Galbeta1-4Glcbeta-Cer; GalNAcbeta1-4(Galbeta1-4GlcNAcbeta1-3)Galbeta1-4Glcbeta-Cer; GalNAcbeta1-4(Galbeta1-4GlcNAcbeta1-3)Galbeta1-4Glcbeta-Cer; GalNAcbeta1-4(Galbeta1-4GlcNAcbeta1-3)Galbeta1-4Glcbeta-Cer(d18:1/16:0); GalNAcbeta1-4(Galbeta1-4GlcNAcbeta1-3)Galbeta1-4Glcbeta-Cer(d18:1/18:0); GalNAcbeta1-4(Galbeta1-4GlcNAcbeta1-3)Galbeta1-4Glcbeta-Cer(d18:1/20:0); GalNAcbeta1-4(Galbeta1-4GlcNAcbeta1-3)Galbeta1-4Glcbeta-Cer(d18:1/22:0); GalNAcbeta1-4(Galbeta1-4GlcNAcbeta1-3)Galbeta1-4Glcbeta-Cer(d18:1/24:0); GalNAcbeta1-4(Galbeta1-4GlcNAcbeta1-3)Galbeta1-4Glcbeta-Cer(d18:1/24:1(15Z)); GalNAcbeta1-4(Galbeta1-4GlcNAcbeta1-3)Galbeta1-4Glcbeta-Cer(d18:1/26:0); GalNAcbeta1-4(Galbeta1-4GlcNAcbeta1-3)Galbeta1-4Glcbeta-Cer(d18:1/26:1(17Z)); GalNAcbeta1-4(KDNalpha2-3)Galbeta1-4Glcbeta-Cer; GalNAcbeta1-4(KDNalpha2-3)Galbeta1-4Glcbeta-Cer(d18:1/16:0); GalNAcbeta1-4(KDNalpha2-3)Galbeta1-4Glcbeta-Cer(d18:1/18:0); GalNAcbeta1-4(KDNalpha2-3)Galbeta1-4Glcbeta-Cer(d18:1/20:0); GalNAcbeta1-4(KDNalpha2-3)Galbeta1-4Glcbeta-Cer(d18:1/22:0); GalNAcbeta1-4(KDNalpha2-3)Galbeta1-4Glcbeta-Cer(d18:1/24:0); GalNAcbeta1-4(KDNalpha2-3)Galbeta1-4Glcbeta-Cer(d18:1/24:1(15Z)); GalNAcbeta1-4(KDNalpha2-3)Galbeta1-4Glcbeta-Cer(d18:1/26:0); GalNAcbeta1-4(KDNalpha2-3)Galbeta1-4Glcbeta-Cer(d18:1/26:1(17Z)); GalNAcbeta1-4(NeuAcalpha2-3)Galbeta1-3GalNAcbeta1-4(NeuAcalpha2-3)Galbeta1-4Glcbeta-Cer; GalNAcbeta1-4(NeuAcalpha2-3)Galbeta1-3GalNAcbeta1-4(NeuAcalpha2-3)Galbeta1-4Glcbeta-Cer(d18:1/16:0); GalNAcbeta1-4(NeuAcalpha2-3)Galbeta1-3GalNAcbeta1-4(NeuAcalpha2-3)Galbeta1-4Glcbeta-Cer(d18:1/18:0); GalNAcbeta1-4(NeuAcalpha2-3)Galbeta1-3GalNAcbeta1-4(NeuAcalpha2-3)Galbeta1-4Glcbeta-Cer(d18:1/20:0); GalNAcbeta1-4(NeuAcalpha2-3)Galbeta1-3GalNAcbeta1-4(NeuAcalpha2-3)Galbeta1-4Glcbeta-Cer(d18:1/22:0); GalNAcbeta1-4(NeuAcalpha2-3)Galbeta1-3GalNAcbeta1-4(NeuAcalpha2-3)Galbeta1-4Glcbeta-Cer(d18:1/24:0); GalNAcbeta1-4(NeuAcalpha2-3)Galbeta1-3GalNAcbeta1-4(NeuAcalpha2-3)Galbeta1-4Glcbeta-Cer(d18:1/24:1(15Z)); GalNAcbeta1-4(NeuAcalpha2-3)Galbeta1-3GalNAcbeta1-4(NeuAcalpha2-3)Galbeta1-4Glcbeta-Cer(d18:1/26:0); GalNAcbeta1-4(NeuAcalpha2-3)Galbeta1-3GalNAcbeta1-4(NeuAcalpha2-3)Galbeta1-4Glcbeta-Cer(d18:1/26:1(17Z)); GalNAcbeta1-4(NeuAcalpha2-3)Galbeta1-3GalNAcbeta1-4(NeuGcalpha2-3)Galbeta1-4Glcbeta-Cer; GalNAcbeta1-4(NeuAcalpha2-3)Galbeta1-3GalNAcbeta1-4(NeuGcalpha2-3)Galbeta1-4Glcbeta-Cer(d18:1/16:0); GalNAcbeta1-4(NeuAcalpha2-3)Galbeta1-3GalNAcbeta1-4(NeuGcalpha2-3)Galbeta1-4Glcbeta-Cer(d18:1/18:0); GalNAcbeta1-4(NeuAcalpha2-3)Galbeta1-3GalNAcbeta1-4(NeuGcalpha2-3)Galbeta1-4Glcbeta-Cer(d18:1/20:0); GalNAcbeta1-4(NeuAcalpha2-3)Galbeta1-3GalNAcbeta1-4(NeuGcalpha2-3)Galbeta1-4Glcbeta-Cer(d18:1/22:0); GalNAcbeta1-4(NeuAcalpha2-3)Galbeta1-3GalNAcbeta1-4(NeuGcalpha2-3)Galbeta1-4Glcbeta-Cer(d18:1/24:0); GalNAcbeta1-4(NeuAcalpha2-3)Galbeta1-3GalNAcbeta1-4(NeuGcalpha2-3)Galbeta1-4Glcbeta-Cer(d18:1/24:1(15Z)); GalNAcbeta1-4(NeuAcalpha2-3)Galbeta1-3GalNAcbeta1-4(NeuGcalpha2-3)Galbeta1-4Glcbeta-Cer(d18:1/26:0); GalNAcbeta1-4(NeuAcalpha2-3)Galbeta1-3GalNAcbeta1-4(NeuGcalpha2-3)Galbeta1-4Glcbeta-Cer(d18:1/26:1(17Z)); GalNAcbeta1-4(NeuAcalpha2-3)Galbeta1-3GalNAcbeta1-4Galbeta1-4Glcbeta-Cer; GalNAcbeta1-4(NeuAcalpha2-3)Galbeta1-3GalNAcbeta1-4Galbeta1-4Glcbeta-Cer(d18:1/16:0); GalNAcbeta1-4(NeuAcalpha2-3)Galbeta1-3GalNAcbeta1-4Galbeta1-4Glcbeta-Cer(d18:1/18:0); GalNAcbeta1-4(NeuAcalpha2-3)Galbeta1-3GalNAcbeta1-4Galbeta1-4Glcbeta-Cer(d18:1/20:0); GalNAcbeta1-4(NeuAcalpha2-3)Galbeta1-3GalNAcbeta1-4Galbeta1-4Glcbeta-Cer(d18:1/22:0); GalNAcbeta1-4(NeuAcalpha2-3)Galbeta1-3GalNAcbeta1-4Galbeta1-4Glcbeta-Cer(d18:1/24:0); GalNAcbeta1-4(NeuAcalpha2-3)Galbeta1-3GalNAcbeta1-4Galbeta1-4Glcbeta-Cer(d18:1/24:1(15Z)); GalNAcbeta1-4(NeuAcalpha2-3)Galbeta1-3GalNAcbeta1-4Galbeta1-4Glcbeta-Cer(d18:1/26:0); GalNAcbeta1-4(NeuAcalpha2-3)Galbeta1-3GalNAcbeta1-4Galbeta1-4Glcbeta-Cer(d18:1/26:1(17Z)); GalNAcbeta1-4(NeuAcalpha2-3)Galbeta1-3GlcNAcbeta1-3(Galbeta1-3GalNAcbeta1-4)Galbeta1-4Glcbeta-Cer; GalNAcbeta1-4(NeuAcalpha2-3)Galbeta1-3GlcNAcbeta1-3(Galbeta1-3GalNAcbeta1-4)Galbeta1-4Glcbeta-Cer(d18:1/16:0); GalNAcbeta1-4(NeuAcalpha2-3)Galbeta1-3GlcNAcbeta1-3(Galbeta1-3GalNAcbeta1-4)Galbeta1-4Glcbeta-Cer(d18:1/18:0); GalNAcbeta1-4(NeuAcalpha2-3)Galbeta1-3GlcNAcbeta1-3(Galbeta1-3GalNAcbeta1-4)Galbeta1-4Glcbeta-Cer(d18:1/20:0); GalNAcbeta1-4(NeuAcalpha2-3)Galbeta1-3GlcNAcbeta1-3(Galbeta1-3GalNAcbeta1-4)Galbeta1-4Glcbeta-Cer(d18:1/22:0); GalNAcbeta1-4(NeuAcalpha2-3)Galbeta1-3GlcNAcbeta1-3(Galbeta1-3GalNAcbeta1-4)Galbeta1-4Glcbeta-Cer(d18:1/24:0); GalNAcbeta1-4(NeuAcalpha2-3)Galbeta1-3GlcNAcbeta1-3(Galbeta1-3GalNAcbeta1-4)Galbeta1-4Glcbeta-Cer(d18:1/24:1(15Z)); GalNAcbeta1-4(NeuAcalpha2-3)Galbeta1-3GlcNAcbeta1-3(Galbeta1-3GalNAcbeta1-4)Galbeta1-4Glcbeta-Cer(d18:1/26:0); GalNAcbeta1-4(NeuAcalpha2-3)Galbeta1-3GlcNAcbeta1-3(Galbeta1-3GalNAcbeta1-4)Galbeta1-4Glcbeta-Cer(d18:1/26:1(17Z)); GalNAcbeta1-4(NeuAcalpha2-3)Galbeta1-3GlcNAcbeta1-3(GalNAcbeta1-4)Galbeta1-4Glcbeta-Cer; GalNAcbeta1-4(NeuAcalpha2-3)Galbeta1-3GlcNAcbeta1-3(GalNAcbeta1-4)Galbeta1-4Glcbeta-Cer(d18:1/16:0); GalNAcbeta1-4(NeuAcalpha2-3)Galbeta1-3GlcNAcbeta1-3(GalNAcbeta1-4)Galbeta1-4Glcbeta-Cer(d18:1/18:0); GalNAcbeta1-4(NeuAcalpha2-3)Galbeta1-3GlcNAcbeta1-3(GalNAcbeta1-4)Galbeta1-4Glcbeta-Cer(d18:1/20:0); GalNAcbeta1-4(NeuAcalpha2-3)Galbeta1-3GlcNAcbeta1-3(GalNAcbeta1-4)Galbeta1-4Glcbeta-Cer(d18:1/22:0); GalNAcbeta1-4(NeuAcalpha2-3)Galbeta1-3GlcNAcbeta1-3(GalNAcbeta1-4)Galbeta1-4Glcbeta-Cer(d18:1/24:0); GalNAcbeta1-4(NeuAcalpha2-3)Galbeta1-3GlcNAcbeta1-3(GalNAcbeta1-4)Galbeta1-4Glcbeta-Cer(d18:1/24:1(15Z)); GalNAcbeta1-4(NeuAcalpha2-3)Galbeta1-3GlcNAcbeta1-3(GalNAcbeta1-4)Galbeta1-4Glcbeta-Cer(d18:1/26:0); GalNAcbeta1-4(NeuAcalpha2-3)Galbeta1-3GlcNAcbeta1-3(GalNAcbeta1-4)Galbeta1-4Glcbeta-Cer(d18:1/26:1(17Z)); GalNAcbeta1-4(NeuAcalpha2-3)Galbeta1-3GlcNAcbeta1-3Galbeta1-4Glcbeta-Cer; GalNAcbeta1-4(NeuAcalpha2-3)Galbeta1-3GlcNAcbeta1-3Galbeta1-4Glcbeta-Cer(d18:1/16:0); GalNAcbeta1-4(NeuAcalpha2-3)Galbeta1-3GlcNAcbeta1-3Galbeta1-4Glcbeta-Cer(d18:1/18:0); GalNAcbeta1-4(NeuAcalpha2-3)Galbeta1-3GlcNAcbeta1-3Galbeta1-4Glcbeta-Cer(d18:1/20:0); GalNAcbeta1-4(NeuAcalpha2-3)Galbeta1-3GlcNAcbeta1-3Galbeta1-4Glcbeta-Cer(d18:1/22:0); GalNAcbeta1-4(NeuAcalpha2-3)Galbeta1-3GlcNAcbeta1-3Galbeta1-4Glcbeta-Cer(d18:1/24:0); GalNAcbeta1-4(NeuAcalpha2-3)Galbeta1-3GlcNAcbeta1-3Galbeta1-4Glcbeta-Cer(d18:1/24:1(15Z)); GalNAcbeta1-4(NeuAcalpha2-3)Galbeta1-3GlcNAcbeta1-3Galbeta1-4Glcbeta-Cer(d18:1/26:0); GalNAcbeta1-4(NeuAcalpha2-3)Galbeta1-3GlcNAcbeta1-3Galbeta1-4Glcbeta-Cer(d18:1/26:1(17Z)); GalNAcbeta1-4(NeuAcalpha2-3)Galbeta1-4Glcbeta-Cer; GalNAcbeta1-4(NeuAcalpha2-3)Galbeta1-4Glcbeta-Cer(d18:1/16:0); GalNAcbeta1-4(NeuAcalpha2-3)Galbeta1-4Glcbeta-Cer(d18:1/18:0); GalNAcbeta1-4(NeuAcalpha2-3)Galbeta1-4Glcbeta-Cer(d18:1/20:0); GalNAcbeta1-4(NeuAcalpha2-3)Galbeta1-4Glcbeta-Cer(d18:1/22:0); GalNAcbeta1-4(NeuAcalpha2-3)Galbeta1-4Glcbeta-Cer(d18:1/24:0); GalNAcbeta1-4(NeuAcalpha2-3)Galbeta1-4Glcbeta-Cer(d18:1/24:1(15Z)); GalNAcbeta1-4(NeuAcalpha2-3)Galbeta1-4Glcbeta-Cer(d18:1/26:0); GalNAcbeta1-4(NeuAcalpha2-3)Galbeta1-4Glcbeta-Cer(d18:1/26:1(17Z)); GalNAcbeta1-4(NeuAcalpha2-3)Galbeta1-4GlcNAcbeta1-3(GalNAcbeta1-4)Galbeta1-4Glcbeta-Cer; GalNAcbeta1-4(NeuAcalpha2-3)Galbeta1-4GlcNAcbeta1-3(GalNAcbeta1-4)Galbeta1-4Glcbeta-Cer(d18:1/16:0); GalNAcbeta1-4(NeuAcalpha2-3)Galbeta1-4GlcNAcbeta1-3(GalNAcbeta1-4)Galbeta1-4Glcbeta-Cer(d18:1/18:0); GalNAcbeta1-4(NeuAcalpha2-3)Galbeta1-4GlcNAcbeta1-3(GalNAcbeta1-4)Galbeta1-4Glcbeta-Cer(d18:1/20:0); GalNAcbeta1-4(NeuAcalpha2-3)Galbeta1-4GlcNAcbeta1-3(GalNAcbeta1-4)Galbeta1-4Glcbeta-Cer(d18:1/22:0); GalNAcbeta1-4(NeuAcalpha2-3)Galbeta1-4GlcNAcbeta1-3(GalNAcbeta1-4)Galbeta1-4Glcbeta-Cer(d18:1/24:0); GalNAcbeta1-4(NeuAcalpha2-3)Galbeta1-4GlcNAcbeta1-3(GalNAcbeta1-4)Galbeta1-4Glcbeta-Cer(d18:1/24:1(15Z)); GalNAcbeta1-4(NeuAcalpha2-3)Galbeta1-4GlcNAcbeta1-3(GalNAcbeta1-4)Galbeta1-4Glcbeta-Cer(d18:1/26:0); GalNAcbeta1-4(NeuAcalpha2-3)Galbeta1-4GlcNAcbeta1-3(GalNAcbeta1-4)Galbeta1-4Glcbeta-Cer(d18:1/26:1(17Z)); GalNAcbeta1-4(NeuAcalpha2-3)Galbeta1-4GlcNAcbeta1-3Galbeta1-4Glcbeta-Cer; GalNAcbeta1-4(NeuAcalpha2-3)Galbeta1-4GlcNAcbeta1-3Galbeta1-4Glcbeta-Cer(d18:1/16:0); GalNAcbeta1-4(NeuAcalpha2-3)Galbeta1-4GlcNAcbeta1-3Galbeta1-4Glcbeta-Cer(d18:1/18:0); GalNAcbeta1-4(NeuAcalpha2-3)Galbeta1-4GlcNAcbeta1-3Galbeta1-4Glcbeta-Cer(d18:1/20:0); GalNAcbeta1-4(NeuAcalpha2-3)Galbeta1-4GlcNAcbeta1-3Galbeta1-4Glcbeta-Cer(d18:1/22:0); GalNAcbeta1-4(NeuAcalpha2-3)Galbeta1-4GlcNAcbeta1-3Galbeta1-4Glcbeta-Cer(d18:1/24:0); GalNAcbeta1-4(NeuAcalpha2-3)Galbeta1-4GlcNAcbeta1-3Galbeta1-4Glcbeta-Cer(d18:1/24:1(15Z)); GalNAcbeta1-4(NeuAcalpha2-3)Galbeta1-4GlcNAcbeta1-3Galbeta1-4Glcbeta-Cer(d18:1/26:0); GalNAcbeta1-4(NeuAcalpha2-3)Galbeta1-4GlcNAcbeta1-3Galbeta1-4Glcbeta-Cer(d18:1/26:1(17Z)); GalNAcbeta1-4(NeuAcalpha2-8NeuAcalpha2-3)Galbeta1-4Glcbeta-Cer; GalNAcbeta1-4(NeuAcalpha2-8NeuAcalpha2-3)Galbeta1-4Glcbeta-Cer(d18:1/16:0); GalNAcbeta1-4(NeuAcalpha2-8NeuAcalpha2-3)Galbeta1-4Glcbeta-Cer(d18:1/18:0); GalNAcbeta1-4(NeuAcalpha2-8NeuAcalpha2-3)Galbeta1-4Glcbeta-Cer(d18:1/20:0); GalNAcbeta1-4(NeuAcalpha2-8NeuAcalpha2-3)Galbeta1-4Glcbeta-Cer(d18:1/22:0); GalNAcbeta1-4(NeuAcalpha2-8NeuAcalpha2-3)Galbeta1-4Glcbeta-Cer(d18:1/24:0); GalNAcbeta1-4(NeuAcalpha2-8NeuAcalpha2-3)Galbeta1-4Glcbeta-Cer(d18:1/24:1(15Z)); GalNAcbeta1-4(NeuAcalpha2-8NeuAcalpha2-3)Galbeta1-4Glcbeta-Cer(d18:1/26:0); GalNAcbeta1-4(NeuAcalpha2-8NeuAcalpha2-3)Galbeta1-4Glcbeta-Cer(d18:1/26:1(17Z)); GalNAcbeta1-4(NeuAcalpha2-8NeuAcalpha2-8NeuAcalpha2-3)Galbeta1-4Glcbeta-Cer; GalNAcbeta1-4(NeuAcalpha2-8NeuAcalpha2-8NeuAcalpha2-3)Galbeta1-4Glcbeta-Cer(d18:1/16:0); GalNAcbeta1-4(NeuAcalpha2-8NeuAcalpha2-8NeuAcalpha2-3)Galbeta1-4Glcbeta-Cer(d18:1/18:0); GalNAcbeta1-4(NeuAcalpha2-8NeuAcalpha2-8NeuAcalpha2-3)Galbeta1-4Glcbeta-Cer(d18:1/20:0); GalNAcbeta1-4(NeuAcalpha2-8NeuAcalpha2-8NeuAcalpha2-3)Galbeta1-4Glcbeta-Cer(d18:1/22:0); GalNAcbeta1-4(NeuAcalpha2-8NeuAcalpha2-8NeuAcalpha2-3)Galbeta1-4Glcbeta-Cer(d18:1/24:0); GalNAcbeta1-4(NeuAcalpha2-8NeuAcalpha2-8NeuAcalpha2-3)Galbeta1-4Glcbeta-Cer(d18:1/24:1(15Z)); GalNAcbeta1-4(NeuAcalpha2-8NeuAcalpha2-8NeuAcalpha2-3)Galbeta1-4Glcbeta-Cer(d18:1/26:0); GalNAcbeta1-4(NeuAcalpha2-8NeuAcalpha2-8NeuAcalpha2-3)Galbeta1-4Glcbeta-Cer(d18:1/26:1(17Z)); GalNAcbeta1-4(NeuGcalpha2-3)Galbeta1-3GalNAcbeta1-4(NeuAcalpha2-3)Galbeta1-4Glcbeta-Cer; GalNAcbeta1-4(NeuGcalpha2-3)Galbeta1-3GalNAcbeta1-4(NeuAcalpha2-3)Galbeta1-4Glcbeta-Cer(d18:1/16:0); GalNAcbeta1-4(NeuGcalpha2-3)Galbeta1-3GalNAcbeta1-4(NeuAcalpha2-3)Galbeta1-4Glcbeta-Cer(d18:1/18:0); GalNAcbeta1-4(NeuGcalpha2-3)Galbeta1-3GalNAcbeta1-4(NeuAcalpha2-3)Galbeta1-4Glcbeta-Cer(d18:1/20:0); GalNAcbeta1-4(NeuGcalpha2-3)Galbeta1-3GalNAcbeta1-4(NeuAcalpha2-3)Galbeta1-4Glcbeta-Cer(d18:1/22:0); GalNAcbeta1-4(NeuGcalpha2-3)Galbeta1-3GalNAcbeta1-4(NeuAcalpha2-3)Galbeta1-4Glcbeta-Cer(d18:1/24:0); GalNAcbeta1-4(NeuGcalpha2-3)Galbeta1-3GalNAcbeta1-4(NeuAcalpha2-3)Galbeta1-4Glcbeta-Cer(d18:1/24:1(15Z)); GalNAcbeta1-4(NeuGcalpha2-3)Galbeta1-3GalNAcbeta1-4(NeuAcalpha2-3)Galbeta1-4Glcbeta-Cer(d18:1/26:0); GalNAcbeta1-4(NeuGcalpha2-3)Galbeta1-3GalNAcbeta1-4(NeuAcalpha2-3)Galbeta1-4Glcbeta-Cer(d18:1/26:1(17Z)); GalNAcbeta1-4(NeuGcalpha2-3)Galbeta1-3GalNAcbeta1-4Galbeta1-4Glcbeta-Cer; GalNAcbeta1-4(NeuGcalpha2-3)Galbeta1-3GalNAcbeta1-4Galbeta1-4Glcbeta-Cer(d18:1/16:0); GalNAcbeta1-4(NeuGcalpha2-3)Galbeta1-3GalNAcbeta1-4Galbeta1-4Glcbeta-Cer(d18:1/18:0); GalNAcbeta1-4(NeuGcalpha2-3)Galbeta1-3GalNAcbeta1-4Galbeta1-4Glcbeta-Cer(d18:1/20:0); GalNAcbeta1-4(NeuGcalpha2-3)Galbeta1-3GalNAcbeta1-4Galbeta1-4Glcbeta-Cer(d18:1/22:0); GalNAcbeta1-4(NeuGcalpha2-3)Galbeta1-3GalNAcbeta1-4Galbeta1-4Glcbeta-Cer(d18:1/24:0); GalNAcbeta1-4(NeuGcalpha2-3)Galbeta1-3GalNAcbeta1-4Galbeta1-4Glcbeta-Cer(d18:1/24:1(15Z)); GalNAcbeta1-4(NeuGcalpha2-3)Galbeta1-3GalNAcbeta1-4Galbeta1-4Glcbeta-Cer(d18:1/26:0); GalNAcbeta1-4(NeuGcalpha2-3)Galbeta1-3GalNAcbeta1-4Galbeta1-4Glcbeta-Cer(d18:1/26:1(17Z)); GalNAcbeta1-4(NeuGcalpha2-3)Galbeta1-4Glcbeta-Cer; GalNAcbeta1-4(NeuGcalpha2-3)Galbeta1-4Glcbeta-Cer(d18:1/16:0); GalNAcbeta1-4(NeuGcalpha2-3)Galbeta1-4Glcbeta-Cer(d18:1/18:0); GalNAcbeta1-4(NeuGcalpha2-3)Galbeta1-4Glcbeta-Cer(d18:1/20:0); GalNAcbeta1-4(NeuGcalpha2-3)Galbeta1-4Glcbeta-Cer(d18:1/22:0); GalNAcbeta1-4(NeuGcalpha2-3)Galbeta1-4Glcbeta-Cer(d18:1/24:0); GalNAcbeta1-4(NeuGcalpha2-3)Galbeta1-4Glcbeta-Cer(d18:1/24:1(15Z)); GalNAcbeta1-4(NeuGcalpha2-3)Galbeta1-4Glcbeta-Cer(d18:1/26:0); GalNAcbeta1-4(NeuGcalpha2-3)Galbeta1-4Glcbeta-Cer(d18:1/26:1(17Z)); GalNAcbeta1-4Galbeta1-3GalNAcbeta1-4(NeuAcalpha2-3)Galbeta1-4Glcbeta-Cer; GalNAcbeta1-4Galbeta1-3GalNAcbeta1-4(NeuAcalpha2-3)Galbeta1-4Glcbeta-Cer(d18:1/16:0); GalNAcbeta1-4Galbeta1-3GalNAcbeta1-4(NeuAcalpha2-3)Galbeta1-4Glcbeta-Cer(d18:1/18:0); GalNAcbeta1-4Galbeta1-3GalNAcbeta1-4(NeuAcalpha2-3)Galbeta1-4Glcbeta-Cer(d18:1/20:0); GalNAcbeta1-4Galbeta1-3GalNAcbeta1-4(NeuAcalpha2-3)Galbeta1-4Glcbeta-Cer(d18:1/22:0); GalNAcbeta1-4Galbeta1-3GalNAcbeta1-4(NeuAcalpha2-3)Galbeta1-4Glcbeta-Cer(d18:1/24:0); GalNAcbeta1-4Galbeta1-3GalNAcbeta1-4(NeuAcalpha2-3)Galbeta1-4Glcbeta-Cer(d18:1/24:1(15Z)); GalNAcbeta1-4Galbeta1-3GalNAcbeta1-4(NeuAcalpha2-3)Galbeta1-4Glcbeta-Cer(d18:1/26:0); GalNAcbeta1-4Galbeta1-3GalNAcbeta1-4(NeuAcalpha2-3)Galbeta1-4Glcbeta-Cer(d18:1/26:1(17Z)); GalNAcbeta1-4Galbeta1-4Glcbeta-Cer; GalNAcbeta1-4Galbeta1-4Glcbeta-Cer(d18:1/16:0); GalNAcbeta1-4Galbeta1-4Glcbeta-Cer(d18:1/18:0); GalNAcbeta1-4Galbeta1-4Glcbeta-Cer(d18:1/20:0); GalNAcbeta1-4Galbeta1-4Glcbeta-Cer(d18:1/22:0); GalNAcbeta1-4Galbeta1-4Glcbeta-Cer(d18:1/24:0); GalNAcbeta1-4Galbeta1-4Glcbeta-Cer(d18:1/24:1(15Z)); GalNAcbeta1-4Galbeta1-4Glcbeta-Cer(d18:1/26:0); GalNAcbeta1-4Galbeta1-4Glcbeta-Cer(d18:1/26:1(17Z)); Glcbeta-Cer; GlcNAcalpha1-3Galbeta1-4GlcNAcbeta1-3Galbeta1-4Glcbeta-Cer; GlcNAcalpha1-3Galbeta1-4GlcNAcbeta1-3Galbeta1-4Glcbeta-Cer(d18:1/16:0); GlcNAcalpha1-3Galbeta1-4GlcNAcbeta1-3Galbeta1-4Glcbeta-Cer(d18:1/18:0); GlcNAcalpha1-3Galbeta1-4GlcNAcbeta1-3Galbeta1-4Glcbeta-Cer(d18:1/20:0); GlcNAcalpha1-3Galbeta1-4GlcNAcbeta1-3Galbeta1-4Glcbeta-Cer(d18:1/22:0); GlcNAcalpha1-3Galbeta1-4GlcNAcbeta1-3Galbeta1-4Glcbeta-Cer(d18:1/24:0); GlcNAcalpha1-3Galbeta1-4GlcNAcbeta1-3Galbeta1-4Glcbeta-Cer(d18:1/24:1(15Z)); GlcNAcalpha1-3Galbeta1-4GlcNAcbeta1-3Galbeta1-4Glcbeta-Cer(d18:1/26:0); GlcNAcalpha1-3Galbeta1-4GlcNAcbeta1-3Galbeta1-4Glcbeta-Cer(d18:1/26:1(17Z)); GlcNAcbeta1-3(GalNAcbeta1-4)Galbeta1-4Glcbeta-Cer; GlcNAcbeta1-3(GalNAcbeta1-4)Galbeta1-4Glcbeta-Cer(d18:1/16:0); GlcNAcbeta1-3(GalNAcbeta1-4)Galbeta1-4Glcbeta-Cer(d18:1/18:0); GlcNAcbeta1-3(GalNAcbeta1-4)Galbeta1-4Glcbeta-Cer(d18:1/20:0); GlcNAcbeta1-3(GalNAcbeta1-4)Galbeta1-4Glcbeta-Cer(d18:1/22:0); GlcNAcbeta1-3(GalNAcbeta1-4)Galbeta1-4Glcbeta-Cer(d18:1/24:0); GlcNAcbeta1-3(GalNAcbeta1-4)Galbeta1-4Glcbeta-Cer(d18:1/24:1(15Z)); GlcNAcbeta1-3(GalNAcbeta1-4)Galbeta1-4Glcbeta-Cer(d18:1/26:0); GlcNAcbeta1-3(GalNAcbeta1-4)Galbeta1-4Glcbeta-Cer(d18:1/26:1(17Z)); GlcNAcbeta1-3Galalpha1-3(GalNAcbeta1-4)Galbeta1-4Glcbeta-Cer; GlcNAcbeta1-3Galalpha1-3(GalNAcbeta1-4)Galbeta1-4Glcbeta-Cer(d18:1/16:0); GlcNAcbeta1-3Galalpha1-3(GalNAcbeta1-4)Galbeta1-4Glcbeta-Cer(d18:1/18:0); GlcNAcbeta1-3Galalpha1-3(GalNAcbeta1-4)Galbeta1-4Glcbeta-Cer(d18:1/20:0); GlcNAcbeta1-3Galalpha1-3(GalNAcbeta1-4)Galbeta1-4Glcbeta-Cer(d18:1/22:0); GlcNAcbeta1-3Galalpha1-3(GalNAcbeta1-4)Galbeta1-4Glcbeta-Cer(d18:1/24:0); GlcNAcbeta1-3Galalpha1-3(GalNAcbeta1-4)Galbeta1-4Glcbeta-Cer(d18:1/24:1(15Z)); GlcNAcbeta1-3Galalpha1-3(GalNAcbeta1-4)Galbeta1-4Glcbeta-Cer(d18:1/26:0); GlcNAcbeta1-3Galalpha1-3(GalNAcbeta1-4)Galbeta1-4Glcbeta-Cer(d18:1/26:1(17Z)); GlcNAcbeta1-3Galalpha1-3Galbeta1-4Glcbeta-Cer; GlcNAcbeta1-3Galalpha1-3Galbeta1-4Glcbeta-Cer(d18:1/16:0); GlcNAcbeta1-3Galalpha1-3Galbeta1-4Glcbeta-Cer(d18:1/18:0); GlcNAcbeta1-3Galalpha1-3Galbeta1-4Glcbeta-Cer(d18:1/20:0); GlcNAcbeta1-3Galalpha1-3Galbeta1-4Glcbeta-Cer(d18:1/22:0); GlcNAcbeta1-3Galalpha1-3Galbeta1-4Glcbeta-Cer(d18:1/24:0); GlcNAcbeta1-3Galalpha1-3Galbeta1-4Glcbeta-Cer(d18:1/24:1(15Z)); GlcNAcbeta1-3Galalpha1-3Galbeta1-4Glcbeta-Cer(d18:1/26:0); GlcNAcbeta1-3Galalpha1-3Galbeta1-4Glcbeta-Cer(d18:1/26:1(17Z)); GlcNAcbeta1-3Galalpha1-4Galbeta1-4Glcbeta-Cer; GlcNAcbeta1-3Galalpha1-4Galbeta1-4Glcbeta-Cer(d18:1/16:0); GlcNAcbeta1-3Galalpha1-4Galbeta1-4Glcbeta-Cer(d18:1/18:0); GlcNAcbeta1-3Galalpha1-4Galbeta1-4Glcbeta-Cer(d18:1/20:0); GlcNAcbeta1-3Galalpha1-4Galbeta1-4Glcbeta-Cer(d18:1/22:0); GlcNAcbeta1-3Galalpha1-4Galbeta1-4Glcbeta-Cer(d18:1/24:0); GlcNAcbeta1-3Galalpha1-4Galbeta1-4Glcbeta-Cer(d18:1/24:1(15Z)); GlcNAcbeta1-3Galalpha1-4Galbeta1-4Glcbeta-Cer(d18:1/26:0); GlcNAcbeta1-3Galalpha1-4Galbeta1-4Glcbeta-Cer(d18:1/26:1(17Z)); GlcNAcbeta1-3Galbeta1-3(Fucalpha1-4)GlcNAcbeta1-3Galbeta1-4Glcbeta-Cer; GlcNAcbeta1-3Galbeta1-3(Fucalpha1-4)GlcNAcbeta1-3Galbeta1-4Glcbeta-Cer(d18:1/16:0); GlcNAcbeta1-3Galbeta1-3(Fucalpha1-4)GlcNAcbeta1-3Galbeta1-4Glcbeta-Cer(d18:1/18:0); GlcNAcbeta1-3Galbeta1-3(Fucalpha1-4)GlcNAcbeta1-3Galbeta1-4Glcbeta-Cer(d18:1/20:0); GlcNAcbeta1-3Galbeta1-3(Fucalpha1-4)GlcNAcbeta1-3Galbeta1-4Glcbeta-Cer(d18:1/22:0); GlcNAcbeta1-3Galbeta1-3(Fucalpha1-4)GlcNAcbeta1-3Galbeta1-4Glcbeta-Cer(d18:1/24:0); GlcNAcbeta1-3Galbeta1-3(Fucalpha1-4)GlcNAcbeta1-3Galbeta1-4Glcbeta-Cer(d18:1/24:1(15Z)); GlcNAcbeta1-3Galbeta1-3(Fucalpha1-4)GlcNAcbeta1-3Galbeta1-4Glcbeta-Cer(d18:1/26:0); GlcNAcbeta1-3Galbeta1-3(Fucalpha1-4)GlcNAcbeta1-3Galbeta1-4Glcbeta-Cer(d18:1/26:1(17Z)); GlcNAcbeta1-3Galbeta1-3GalNAcbeta1-4(NeuAcalpha2-3)Galbeta1-4Glcbeta-Cer; GlcNAcbeta1-3Galbeta1-3GalNAcbeta1-4(NeuAcalpha2-3)Galbeta1-4Glcbeta-Cer(d18:1/16:0); GlcNAcbeta1-3Galbeta1-3GalNAcbeta1-4(NeuAcalpha2-3)Galbeta1-4Glcbeta-Cer(d18:1/18:0); GlcNAcbeta1-3Galbeta1-3GalNAcbeta1-4(NeuAcalpha2-3)Galbeta1-4Glcbeta-Cer(d18:1/20:0); GlcNAcbeta1-3Galbeta1-3GalNAcbeta1-4(NeuAcalpha2-3)Galbeta1-4Glcbeta-Cer(d18:1/22:0); GlcNAcbeta1-3Galbeta1-3GalNAcbeta1-4(NeuAcalpha2-3)Galbeta1-4Glcbeta-Cer(d18:1/24:0); GlcNAcbeta1-3Galbeta1-3GalNAcbeta1-4(NeuAcalpha2-3)Galbeta1-4Glcbeta-Cer(d18:1/24:1(15Z)); GlcNAcbeta1-3Galbeta1-3GalNAcbeta1-4(NeuAcalpha2-3)Galbeta1-4Glcbeta-Cer(d18:1/26:0); GlcNAcbeta1-3Galbeta1-3GalNAcbeta1-4(NeuAcalpha2-3)Galbeta1-4Glcbeta-Cer(d18:1/26:1(17Z)); GlcNAcbeta1-3Galbeta1-3GalNAcbeta1-4Galbeta1-4Glcbeta-Cer; GlcNAcbeta1-3Galbeta1-3GalNAcbeta1-4Galbeta1-4Glcbeta-Cer(d18:1/16:0); GlcNAcbeta1-3Galbeta1-3GalNAcbeta1-4Galbeta1-4Glcbeta-Cer(d18:1/18:0); GlcNAcbeta1-3Galbeta1-3GalNAcbeta1-4Galbeta1-4Glcbeta-Cer(d18:1/20:0); GlcNAcbeta1-3Galbeta1-3GalNAcbeta1-4Galbeta1-4Glcbeta-Cer(d18:1/22:0); GlcNAcbeta1-3Galbeta1-3GalNAcbeta1-4Galbeta1-4Glcbeta-Cer(d18:1/24:0); GlcNAcbeta1-3Galbeta1-3GalNAcbeta1-4Galbeta1-4Glcbeta-Cer(d18:1/24:1(15Z)); GlcNAcbeta1-3Galbeta1-3GalNAcbeta1-4Galbeta1-4Glcbeta-Cer(d18:1/26:0); GlcNAcbeta1-3Galbeta1-3GalNAcbeta1-4Galbeta1-4Glcbeta-Cer(d18:1/26:1(17Z)); GlcNAcbeta1-3Galbeta1-3GlcNAcbeta1-3Galbeta1-4Glcbeta-Cer; GlcNAcbeta1-3Galbeta1-3GlcNAcbeta1-3Galbeta1-4Glcbeta-Cer(d18:1/16:0); GlcNAcbeta1-3Galbeta1-3GlcNAcbeta1-3Galbeta1-4Glcbeta-Cer(d18:1/18:0); GlcNAcbeta1-3Galbeta1-3GlcNAcbeta1-3Galbeta1-4Glcbeta-Cer(d18:1/20:0); GlcNAcbeta1-3Galbeta1-3GlcNAcbeta1-3Galbeta1-4Glcbeta-Cer(d18:1/22:0); GlcNAcbeta1-3Galbeta1-3GlcNAcbeta1-3Galbeta1-4Glcbeta-Cer(d18:1/24:0); GlcNAcbeta1-3Galbeta1-3GlcNAcbeta1-3Galbeta1-4Glcbeta-Cer(d18:1/24:1(15Z)); GlcNAcbeta1-3Galbeta1-3GlcNAcbeta1-3Galbeta1-4Glcbeta-Cer(d18:1/26:0); GlcNAcbeta1-3Galbeta1-3GlcNAcbeta1-3Galbeta1-4Glcbeta-Cer(d18:1/26:1(17Z)); GlcNAcbeta1-3Galbeta1-4(Fucalpha1-3)GlcNAcbeta1-3Galbeta1-4Glcbeta-Cer; GlcNAcbeta1-3Galbeta1-4(Fucalpha1-3)GlcNAcbeta1-3Galbeta1-4Glcbeta-Cer(d18:1/16:0); GlcNAcbeta1-3Galbeta1-4(Fucalpha1-3)GlcNAcbeta1-3Galbeta1-4Glcbeta-Cer(d18:1/18:0); GlcNAcbeta1-3Galbeta1-4(Fucalpha1-3)GlcNAcbeta1-3Galbeta1-4Glcbeta-Cer(d18:1/20:0); GlcNAcbeta1-3Galbeta1-4(Fucalpha1-3)GlcNAcbeta1-3Galbeta1-4Glcbeta-Cer(d18:1/22:0); GlcNAcbeta1-3Galbeta1-4(Fucalpha1-3)GlcNAcbeta1-3Galbeta1-4Glcbeta-Cer(d18:1/24:0); GlcNAcbeta1-3Galbeta1-4(Fucalpha1-3)GlcNAcbeta1-3Galbeta1-4Glcbeta-Cer(d18:1/24:1(15Z)); GlcNAcbeta1-3Galbeta1-4(Fucalpha1-3)GlcNAcbeta1-3Galbeta1-4Glcbeta-Cer(d18:1/26:0); GlcNAcbeta1-3Galbeta1-4(Fucalpha1-3)GlcNAcbeta1-3Galbeta1-4Glcbeta-Cer(d18:1/26:1(17Z)); GlcNAcbeta1-3Galbeta1-4(Fucalpha1-3)GlcNAcbeta1-3Galbeta1-4GlcNAcbeta1-3Galbeta1-4Glcbeta-Cer; GlcNAcbeta1-3Galbeta1-4(Fucalpha1-3)GlcNAcbeta1-3Galbeta1-4GlcNAcbeta1-3Galbeta1-4Glcbeta-Cer(d18:1/16:0); GlcNAcbeta1-3Galbeta1-4(Fucalpha1-3)GlcNAcbeta1-3Galbeta1-4GlcNAcbeta1-3Galbeta1-4Glcbeta-Cer(d18:1/18:0); GlcNAcbeta1-3Galbeta1-4(Fucalpha1-3)GlcNAcbeta1-3Galbeta1-4GlcNAcbeta1-3Galbeta1-4Glcbeta-Cer(d18:1/20:0); GlcNAcbeta1-3Galbeta1-4(Fucalpha1-3)GlcNAcbeta1-3Galbeta1-4GlcNAcbeta1-3Galbeta1-4Glcbeta-Cer(d18:1/22:0); GlcNAcbeta1-3Galbeta1-4(Fucalpha1-3)GlcNAcbeta1-3Galbeta1-4GlcNAcbeta1-3Galbeta1-4Glcbeta-Cer(d18:1/24:0); GlcNAcbeta1-3Galbeta1-4(Fucalpha1-3)GlcNAcbeta1-3Galbeta1-4GlcNAcbeta1-3Galbeta1-4Glcbeta-Cer(d18:1/24:1(15Z)); GlcNAcbeta1-3Galbeta1-4(Fucalpha1-3)GlcNAcbeta1-3Galbeta1-4GlcNAcbeta1-3Galbeta1-4Glcbeta-Cer(d18:1/26:0); GlcNAcbeta1-3Galbeta1-4(Fucalpha1-3)GlcNAcbeta1-3Galbeta1-4GlcNAcbeta1-3Galbeta1-4Glcbeta-Cer(d18:1/26:1(17Z)); GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer; GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/16:0); GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/18:0); GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/20:0); GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/22:0); GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/24:0); GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/24:1(15Z)); GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/26:0); GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/26:1(17Z)); GlcNAcbeta1-3Galbeta1-4Glcbeta-Cer; GlcNAcbeta1-3Galbeta1-4Glcbeta-Cer(d18:1/16:0); GlcNAcbeta1-3Galbeta1-4Glcbeta-Cer(d18:1/18:0); GlcNAcbeta1-3Galbeta1-4Glcbeta-Cer(d18:1/20:0); GlcNAcbeta1-3Galbeta1-4Glcbeta-Cer(d18:1/22:0); GlcNAcbeta1-3Galbeta1-4Glcbeta-Cer(d18:1/24:0); GlcNAcbeta1-3Galbeta1-4Glcbeta-Cer(d18:1/24:1(15Z)); GlcNAcbeta1-3Galbeta1-4Glcbeta-Cer(d18:1/26:0); GlcNAcbeta1-3Galbeta1-4Glcbeta-Cer(d18:1/26:1(17Z)); GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer; GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/16:0); GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/18:0); GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/20:0); GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/22:0); GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:0); GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:1(15Z)); GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:0); GlcNAcbeta1-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:1(17Z)); GlcNAcbeta1-3Galbeta1-4GlcNAcbeta1-3Galbeta1-4Glcbeta-Cer; GlcNAcbeta1-3Galbeta1-4GlcNAcbeta1-3Galbeta1-4Glcbeta-Cer(d18:1/16:0); GlcNAcbeta1-3Galbeta1-4GlcNAcbeta1-3Galbeta1-4Glcbeta-Cer(d18:1/18:0); GlcNAcbeta1-3Galbeta1-4GlcNAcbeta1-3Galbeta1-4Glcbeta-Cer(d18:1/20:0); GlcNAcbeta1-3Galbeta1-4GlcNAcbeta1-3Galbeta1-4Glcbeta-Cer(d18:1/22:0); GlcNAcbeta1-3Galbeta1-4GlcNAcbeta1-3Galbeta1-4Glcbeta-Cer(d18:1/24:0); GlcNAcbeta1-3Galbeta1-4GlcNAcbeta1-3Galbeta1-4Glcbeta-Cer(d18:1/24:1(15Z)); GlcNAcbeta1-3Galbeta1-4GlcNAcbeta1-3Galbeta1-4Glcbeta-Cer(d18:1/26:0); GlcNAcbeta1-3Galbeta1-4GlcNAcbeta1-3Galbeta1-4Glcbeta-Cer(d18:1/26:1(17Z)); GlcNAcbeta1-3Galbeta1-4GlcNAcbeta1-3Galbeta1-4GlcNAcbeta1-3Galbeta1-4Glcbeta-Cer; GlcNAcbeta1-3Galbeta1-4GlcNAcbeta1-3Galbeta1-4GlcNAcbeta1-3Galbeta1-4Glcbeta-Cer(d18:1/16:0); GlcNAcbeta1-3Galbeta1-4GlcNAcbeta1-3Galbeta1-4GlcNAcbeta1-3Galbeta1-4Glcbeta-Cer(d18:1/18:0); GlcNAcbeta1-3Galbeta1-4GlcNAcbeta1-3Galbeta1-4GlcNAcbeta1-3Galbeta1-4Glcbeta-Cer(d18:1/20:0); GlcNAcbeta1-3Galbeta1-4GlcNAcbeta1-3Galbeta1-4GlcNAcbeta1-3Galbeta1-4Glcbeta-Cer(d18:1/22:0); GlcNAcbeta1-3Galbeta1-4GlcNAcbeta1-3Galbeta1-4GlcNAcbeta1-3Galbeta1-4Glcbeta-Cer(d18:1/24:0); GlcNAcbeta1-3Galbeta1-4GlcNAcbeta1-3Galbeta1-4GlcNAcbeta1-3Galbeta1-4Glcbeta-Cer(d18:1/24:1(15Z)); GlcNAcbeta1-3Galbeta1-4GlcNAcbeta1-3Galbeta1-4GlcNAcbeta1-3Galbeta1-4Glcbeta-Cer(d18:1/26:0); GlcNAcbeta1-3Galbeta1-4GlcNAcbeta1-3Galbeta1-4GlcNAcbeta1-3Galbeta1-4Glcbeta-Cer(d18:1/26:1(17Z)); GlcNAcbeta1-4Manbeta1-4Glcbeta-Cer; GlcNAcbeta1-4Manbeta1-4Glcbeta-Cer(d18:1/16:0); GlcNAcbeta1-4Manbeta1-4Glcbeta-Cer(d18:1/18:0); GlcNAcbeta1-4Manbeta1-4Glcbeta-Cer(d18:1/20:0); GlcNAcbeta1-4Manbeta1-4Glcbeta-Cer(d18:1/22:0); GlcNAcbeta1-4Manbeta1-4Glcbeta-Cer(d18:1/24:0); GlcNAcbeta1-4Manbeta1-4Glcbeta-Cer(d18:1/24:1(15Z)); GlcNAcbeta1-4Manbeta1-4Glcbeta-Cer(d18:1/26:0); GlcNAcbeta1-4Manbeta1-4Glcbeta-Cer(d18:1/26:1(17Z)); GlcNAcbeta1-6GalNAcbeta1-3Galalpha1-4Galbeta1-4Glcbeta-Cer; GlcNAcbeta1-6GalNAcbeta1-3Galalpha1-4Galbeta1-4Glcbeta-Cer(d18:1/16:0); GlcNAcbeta1-6GalNAcbeta1-3Galalpha1-4Galbeta1-4Glcbeta-Cer(d18:1/18:0); GlcNAcbeta1-6GalNAcbeta1-3Galalpha1-4Galbeta1-4Glcbeta-Cer(d18:1/20:0); GlcNAcbeta1-6GalNAcbeta1-3Galalpha1-4Galbeta1-4Glcbeta-Cer(d18:1/22:0); GlcNAcbeta1-6GalNAcbeta1-3Galalpha1-4Galbeta1-4Glcbeta-Cer(d18:1/24:0); GlcNAcbeta1-6GalNAcbeta1-3Galalpha1-4Galbeta1-4Glcbeta-Cer(d18:1/24:1(15Z)); GlcNAcbeta1-6GalNAcbeta1-3Galalpha1-4Galbeta1-4Glcbeta-Cer(d18:1/26:0); GlcNAcbeta1-6GalNAcbeta1-3Galalpha1-4Galbeta1-4Glcbeta-Cer(d18:1/26:1(17Z)); heptadecasphing-4-enine; heptadecasphing-4-enine-1-phosphate; heptadecasphing-4-enine-1-phosphocholine; heptadecasphinganine; heptadecasphinganine-1-phosphate; Hexadecaphing-4-enine-1-phosphate; Hexadecaphinganine-1-phosphate; Hexadecasphing-4E-enine; hexadecasphinganine; HSO3-3Galalpha1-4Galbeta-Cer; HSO3-3Galbeta1-3GalNAcbeta1-3Galalpha1-3Galbeta1-4Glcbeta-Cer; HSO3-3Galbeta1-3GalNAcbeta1-3Galalpha1-4Galbeta1-4Glcbeta-Cer; HSO3-3Galbeta1-3GalNAcbeta1-4Galalpha1-3Galbeta1-4Glcbeta-Cer; HSO3-3Galbeta1-3GalNAcbeta1-4Galbeta1-4Glcbeta-Cer; HSO3-3Galbeta1-3GalNAcbeta1-4HSO3-3Galbeta1-4Glcbeta-Cer; HSO3-3Galbeta1-4Glcbeta-Cer; HSO3-3Galbeta-Cer; HSO3-3GalNAcbeta1-3Galalpha1-3Galbeta1-4Glcbeta-Cer; HSO3-3GalNAcbeta1-3Galalpha1-4Galbeta1-4Glcbeta-Cer; HSO3-3GalNAcbeta1-4Galalpha1-3Galbeta1-4Glcbeta-Cer; HSO3-3GlcAbeta1-3Galbeta1-4GlcNAcbeta1-3Galbeta1-4Glcbeta-Cer; HSO3-3GlcAbeta1-3Galbeta1-4GlcNAcbeta1-3Galbeta1-4GlcNAcbeta1-3Galbeta1-4Glcbeta-Cer; HSO3-6GlcNAcbeta1-3Galbeta1-4Glcbeta-Cer; KDNalpha2-3Galbeta1-3(KDNalpha2-6)GalNAcbeta1-4Galbeta1-4Glcbeta-Cer; KDNalpha2-3Galbeta1-3(KDNalpha2-6)GalNAcbeta1-4Galbeta1-4Glcbeta-Cer(d18:1/16:0); KDNalpha2-3Galbeta1-3(KDNalpha2-6)GalNAcbeta1-4Galbeta1-4Glcbeta-Cer(d18:1/18:0); KDNalpha2-3Galbeta1-3(KDNalpha2-6)GalNAcbeta1-4Galbeta1-4Glcbeta-Cer(d18:1/20:0); KDNalpha2-3Galbeta1-3(KDNalpha2-6)GalNAcbeta1-4Galbeta1-4Glcbeta-Cer(d18:1/22:0); KDNalpha2-3Galbeta1-3(KDNalpha2-6)GalNAcbeta1-4Galbeta1-4Glcbeta-Cer(d18:1/24:0); KDNalpha2-3Galbeta1-3(KDNalpha2-6)GalNAcbeta1-4Galbeta1-4Glcbeta-Cer(d18:1/24:1(15Z)); KDNalpha2-3Galbeta1-3(KDNalpha2-6)GalNAcbeta1-4Galbeta1-4Glcbeta-Cer(d18:1/26:0); KDNalpha2-3Galbeta1-3(KDNalpha2-6)GalNAcbeta1-4Galbeta1-4Glcbeta-Cer(d18:1/26:1(17Z)); KDNalpha2-3Galbeta1-3(NeuAcalpha2-6)GalNAcbeta1-4Galbeta1-4Glcbeta-Cer; KDNalpha2-3Galbeta1-3(NeuAcalpha2-6)GalNAcbeta1-4Galbeta1-4Glcbeta-Cer(d18:1/16:0); KDNalpha2-3Galbeta1-3(NeuAcalpha2-6)GalNAcbeta1-4Galbeta1-4Glcbeta-Cer(d18:1/18:0); KDNalpha2-3Galbeta1-3(NeuAcalpha2-6)GalNAcbeta1-4Galbeta1-4Glcbeta-Cer(d18:1/20:0); KDNalpha2-3Galbeta1-3(NeuAcalpha2-6)GalNAcbeta1-4Galbeta1-4Glcbeta-Cer(d18:1/22:0); KDNalpha2-3Galbeta1-3(NeuAcalpha2-6)GalNAcbeta1-4Galbeta1-4Glcbeta-Cer(d18:1/24:0); KDNalpha2-3Galbeta1-3(NeuAcalpha2-6)GalNAcbeta1-4Galbeta1-4Glcbeta-Cer(d18:1/24:1(15Z)); KDNalpha2-3Galbeta1-3(NeuAcalpha2-6)GalNAcbeta1-4Galbeta1-4Glcbeta-Cer(d18:1/26:0); KDNalpha2-3Galbeta1-3(NeuAcalpha2-6)GalNAcbeta1-4Galbeta1-4Glcbeta-Cer(d18:1/26:1(17Z)); KDNalpha2-3Galbeta1-3GalNAcbeta1-4(KDNalpha2-3)Galbeta1-4Glcbeta-Cer; KDNalpha2-3Galbeta1-3GalNAcbeta1-4(KDNalpha2-3)Galbeta1-4Glcbeta-Cer(d18:1/16:0); KDNalpha2-3Galbeta1-3GalNAcbeta1-4(KDNalpha2-3)Galbeta1-4Glcbeta-Cer(d18:1/18:0); KDNalpha2-3Galbeta1-3GalNAcbeta1-4(KDNalpha2-3)Galbeta1-4Glcbeta-Cer(d18:1/20:0); KDNalpha2-3Galbeta1-3GalNAcbeta1-4(KDNalpha2-3)Galbeta1-4Glcbeta-Cer(d18:1/22:0); KDNalpha2-3Galbeta1-3GalNAcbeta1-4(KDNalpha2-3)Galbeta1-4Glcbeta-Cer(d18:1/24:0); KDNalpha2-3Galbeta1-3GalNAcbeta1-4(KDNalpha2-3)Galbeta1-4Glcbeta-Cer(d18:1/24:1(15Z)); KDNalpha2-3Galbeta1-3GalNAcbeta1-4(KDNalpha2-3)Galbeta1-4Glcbeta-Cer(d18:1/26:0); KDNalpha2-3Galbeta1-3GalNAcbeta1-4(KDNalpha2-3)Galbeta1-4Glcbeta-Cer(d18:1/26:1(17Z)); KDNalpha2-3Galbeta1-3GalNAcbeta1-4(NeuAcalpha2-3)Galbeta1-4Glcbeta-Cer; KDNalpha2-3Galbeta1-3GalNAcbeta1-4(NeuAcalpha2-3)Galbeta1-4Glcbeta-Cer(d18:1/16:0); KDNalpha2-3Galbeta1-3GalNAcbeta1-4(NeuAcalpha2-3)Galbeta1-4Glcbeta-Cer(d18:1/18:0); KDNalpha2-3Galbeta1-3GalNAcbeta1-4(NeuAcalpha2-3)Galbeta1-4Glcbeta-Cer(d18:1/20:0); KDNalpha2-3Galbeta1-3GalNAcbeta1-4(NeuAcalpha2-3)Galbeta1-4Glcbeta-Cer(d18:1/22:0); KDNalpha2-3Galbeta1-3GalNAcbeta1-4(NeuAcalpha2-3)Galbeta1-4Glcbeta-Cer(d18:1/24:0); KDNalpha2-3Galbeta1-3GalNAcbeta1-4(NeuAcalpha2-3)Galbeta1-4Glcbeta-Cer(d18:1/24:1(15Z)); KDNalpha2-3Galbeta1-3GalNAcbeta1-4(NeuAcalpha2-3)Galbeta1-4Glcbeta-Cer(d18:1/26:0); KDNalpha2-3Galbeta1-3GalNAcbeta1-4(NeuAcalpha2-3)Galbeta1-4Glcbeta-Cer(d18:1/26:1(17Z)); KDNalpha2-3Galbeta1-3GalNAcbeta1-4Galbeta1-4Glcbeta-Cer; KDNalpha2-3Galbeta1-3GalNAcbeta1-4Galbeta1-4Glcbeta-Cer(d18:1/16:0); KDNalpha2-3Galbeta1-3GalNAcbeta1-4Galbeta1-4Glcbeta-Cer(d18:1/18:0); KDNalpha2-3Galbeta1-3GalNAcbeta1-4Galbeta1-4Glcbeta-Cer(d18:1/20:0); KDNalpha2-3Galbeta1-3GalNAcbeta1-4Galbeta1-4Glcbeta-Cer(d18:1/22:0); KDNalpha2-3Galbeta1-3GalNAcbeta1-4Galbeta1-4Glcbeta-Cer(d18:1/24:0); KDNalpha2-3Galbeta1-3GalNAcbeta1-4Galbeta1-4Glcbeta-Cer(d18:1/24:1(15Z)); KDNalpha2-3Galbeta1-3GalNAcbeta1-4Galbeta1-4Glcbeta-Cer(d18:1/26:0); KDNalpha2-3Galbeta1-3GalNAcbeta1-4Galbeta1-4Glcbeta-Cer(d18:1/26:1(17Z)); KDNalpha2-3Galbeta1-3GlcNAcbeta1-3Galbeta1-4Glcbeta-Cer; KDNalpha2-3Galbeta1-3GlcNAcbeta1-3Galbeta1-4Glcbeta-Cer(d18:1/16:0); KDNalpha2-3Galbeta1-3GlcNAcbeta1-3Galbeta1-4Glcbeta-Cer(d18:1/18:0); KDNalpha2-3Galbeta1-3GlcNAcbeta1-3Galbeta1-4Glcbeta-Cer(d18:1/20:0); KDNalpha2-3Galbeta1-3GlcNAcbeta1-3Galbeta1-4Glcbeta-Cer(d18:1/22:0); KDNalpha2-3Galbeta1-3GlcNAcbeta1-3Galbeta1-4Glcbeta-Cer(d18:1/24:0); KDNalpha2-3Galbeta1-3GlcNAcbeta1-3Galbeta1-4Glcbeta-Cer(d18:1/24:1(15Z)); KDNalpha2-3Galbeta1-3GlcNAcbeta1-3Galbeta1-4Glcbeta-Cer(d18:1/26:0); KDNalpha2-3Galbeta1-3GlcNAcbeta1-3Galbeta1-4Glcbeta-Cer(d18:1/26:1(17Z)); KDNalpha2-3Galbeta1-4(Fucalpha1-3)GlcNAcbeta1-3Galbeta1-4Glcbeta-Cer; KDNalpha2-3Galbeta1-4(Fucalpha1-3)GlcNAcbeta1-3Galbeta1-4Glcbeta-Cer(d18:1/16:0); KDNalpha2-3Galbeta1-4(Fucalpha1-3)GlcNAcbeta1-3Galbeta1-4Glcbeta-Cer(d18:1/18:0); KDNalpha2-3Galbeta1-4(Fucalpha1-3)GlcNAcbeta1-3Galbeta1-4Glcbeta-Cer(d18:1/20:0); KDNalpha2-3Galbeta1-4(Fucalpha1-3)GlcNAcbeta1-3Galbeta1-4Glcbeta-Cer(d18:1/22:0); KDNalpha2-3Galbeta1-4(Fucalpha1-3)GlcNAcbeta1-3Galbeta1-4Glcbeta-Cer(d18:1/24:0); KDNalpha2-3Galbeta1-4(Fucalpha1-3)GlcNAcbeta1-3Galbeta1-4Glcbeta-Cer(d18:1/24:1(15Z)); KDNalpha2-3Galbeta1-4(Fucalpha1-3)GlcNAcbeta1-3Galbeta1-4Glcbeta-Cer(d18:1/26:0); KDNalpha2-3Galbeta1-4(Fucalpha1-3)GlcNAcbeta1-3Galbeta1-4Glcbeta-Cer(d18:1/26:1(17Z)); KDNalpha2-3Galbeta1-4Glcbeta-Cer; KDNalpha2-3Galbeta1-4Glcbeta-Cer(d18:1/16:0); KDNalpha2-3Galbeta1-4Glcbeta-Cer(d18:1/18:0); KDNalpha2-3Galbeta1-4Glcbeta-Cer(d18:1/20:0); KDNalpha2-3Galbeta1-4Glcbeta-Cer(d18:1/22:0); KDNalpha2-3Galbeta1-4Glcbeta-Cer(d18:1/24:0); KDNalpha2-3Galbeta1-4Glcbeta-Cer(d18:1/24:1(15Z)); KDNalpha2-3Galbeta1-4Glcbeta-Cer(d18:1/26:0); KDNalpha2-3Galbeta1-4Glcbeta-Cer(d18:1/26:1(17Z)); KDNalpha2-3Galbeta1-4GlcNAcbeta1-3Galbeta1-4Glcbeta-Cer; KDNalpha2-3Galbeta1-4GlcNAcbeta1-3Galbeta1-4Glcbeta-Cer(d18:1/16:0); KDNalpha2-3Galbeta1-4GlcNAcbeta1-3Galbeta1-4Glcbeta-Cer(d18:1/18:0); KDNalpha2-3Galbeta1-4GlcNAcbeta1-3Galbeta1-4Glcbeta-Cer(d18:1/20:0); KDNalpha2-3Galbeta1-4GlcNAcbeta1-3Galbeta1-4Glcbeta-Cer(d18:1/22:0); KDNalpha2-3Galbeta1-4GlcNAcbeta1-3Galbeta1-4Glcbeta-Cer(d18:1/24:0); KDNalpha2-3Galbeta1-4GlcNAcbeta1-3Galbeta1-4Glcbeta-Cer(d18:1/24:1(15Z)); KDNalpha2-3Galbeta1-4GlcNAcbeta1-3Galbeta1-4Glcbeta-Cer(d18:1/26:0); KDNalpha2-3Galbeta1-4GlcNAcbeta1-3Galbeta1-4Glcbeta-Cer(d18:1/26:1(17Z)); KDNalpha2-6Galbeta1-3GlcNAcbeta1-3Galbeta1-4Glcbeta-Cer; KDNalpha2-6Galbeta1-3GlcNAcbeta1-3Galbeta1-4Glcbeta-Cer(d18:1/16:0); KDNalpha2-6Galbeta1-3GlcNAcbeta1-3Galbeta1-4Glcbeta-Cer(d18:1/18:0); KDNalpha2-6Galbeta1-3GlcNAcbeta1-3Galbeta1-4Glcbeta-Cer(d18:1/20:0); KDNalpha2-6Galbeta1-3GlcNAcbeta1-3Galbeta1-4Glcbeta-Cer(d18:1/22:0); KDNalpha2-6Galbeta1-3GlcNAcbeta1-3Galbeta1-4Glcbeta-Cer(d18:1/24:0); KDNalpha2-6Galbeta1-3GlcNAcbeta1-3Galbeta1-4Glcbeta-Cer(d18:1/24:1(15Z)); KDNalpha2-6Galbeta1-3GlcNAcbeta1-3Galbeta1-4Glcbeta-Cer(d18:1/26:0); KDNalpha2-6Galbeta1-3GlcNAcbeta1-3Galbeta1-4Glcbeta-Cer(d18:1/26:1(17Z)); KDNalpha2-6Galbeta1-4GlcNAcbeta1-3Galbeta1-4Glcbeta-Cer; KDNalpha2-6Galbeta1-4GlcNAcbeta1-3Galbeta1-4Glcbeta-Cer(d18:1/16:0); KDNalpha2-6Galbeta1-4GlcNAcbeta1-3Galbeta1-4Glcbeta-Cer(d18:1/18:0); KDNalpha2-6Galbeta1-4GlcNAcbeta1-3Galbeta1-4Glcbeta-Cer(d18:1/20:0); KDNalpha2-6Galbeta1-4GlcNAcbeta1-3Galbeta1-4Glcbeta-Cer(d18:1/22:0); KDNalpha2-6Galbeta1-4GlcNAcbeta1-3Galbeta1-4Glcbeta-Cer(d18:1/24:0); KDNalpha2-6Galbeta1-4GlcNAcbeta1-3Galbeta1-4Glcbeta-Cer(d18:1/24:1(15Z)); KDNalpha2-6Galbeta1-4GlcNAcbeta1-3Galbeta1-4Glcbeta-Cer(d18:1/26:0); KDNalpha2-6Galbeta1-4GlcNAcbeta1-3Galbeta1-4Glcbeta-Cer(d18:1/26:1(17Z)); Manalpha1-3Manbeta1-4Glcbeta-Cer; Manalpha1-3Manbeta1-4Glcbeta-Cer(d18:1/16:0); Manalpha1-3Manbeta1-4Glcbeta-Cer(d18:1/18:0); Manalpha1-3Manbeta1-4Glcbeta-Cer(d18:1/20:0); Manalpha1-3Manbeta1-4Glcbeta-Cer(d18:1/22:0); Manalpha1-3Manbeta1-4Glcbeta-Cer(d18:1/24:0); Manalpha1-3Manbeta1-4Glcbeta-Cer(d18:1/24:1(15Z)); Manalpha1-3Manbeta1-4Glcbeta-Cer(d18:1/26:0); Manalpha1-3Manbeta1-4Glcbeta-Cer(d18:1/26:1(17Z)); Manbeta1-4Glcbeta-Cer; Manbeta1-4Glcbeta-Cer(d18:1/16:0); Manbeta1-4Glcbeta-Cer(d18:1/18:0); Manbeta1-4Glcbeta-Cer(d18:1/20:0); Manbeta1-4Glcbeta-Cer(d18:1/22:0); Manbeta1-4Glcbeta-Cer(d18:1/24:0); Manbeta1-4Glcbeta-Cer(d18:1/24:1(15Z)); Manbeta1-4Glcbeta-Cer(d18:1/26:0); Manbeta1-4Glcbeta-Cer(d18:1/26:1(17Z)); methyl 3-(13,13-dibromotrideca-1E,12-dienyl)-2H-azirine-2S-carboxylate; methyl 3-(pentadec-1E-enyl)-2H-azirine-2S-carboxylate; N-(11Z-eicosenoyl)-1-beta-galactosyl-4E,14Z-sphingadienine; N-(11Z-eicosenoyl)-1beta-glucosyl-4E,14Z-sphingadienine; N-(11Z-eicosenoyl)-4E,12Z-sphingadienine-1-phosphocholine; N-(11Z-eicosenoyl)-4E,14Z-sphingadienine; N-(11Z-eicosenoyl)-4E,6E-hexadecasphingadienine; N-(11Z-eicosenoyl)-4E,6E-hexadecasphingadienine-1-phosphoethanolamine; N-(11Z-eicosenoyl)-4E,6E-tetradecasphingadienine; N-(11Z-eicosenoyl)-4E,6E-tetradecasphingadienine-1-phosphoethanolamine; N-(11Z-eicosenoyl)-hexadecasphing-4-enine; N-(11Z-eicosenoyl)-hexadecasphing-4-enine-1-phosphocholine; N-(11Z-eicosenoyl)-sphing-4-enine-1-phosphocholine; N-(11Z-eicosenoyl)-tetradecasphing-4-enine; N-(11Z-eicosenoyl)-tetradecasphing-4-enine-1-phosphoethanolamine; N-(13-methyltetradecanoyl)-15-methylhexadecasphinganine-1-phospho-(1′-myo-inositol); N-(13-methyltetradecanoyl)-15-methylhexadecasphinganine-1-phospho-beta-D-mannoside; N-(13-methyltetradecanoyl)-15-methylhexadecasphinganine-1-phosphoethanolamine; N-(13Z-docosenoyl)-4E,14Z-sphingadienine-1-phosphocholine; N-(13Z-docosenoyl)-4E,6E-hexadecasphingadienine; N-(13Z-docosenoyl)-4E,6E-hexadecasphingadienine-1-phosphoethanolamine; N-(13Z-docosenoyl)-4E,6E-tetradecasphingadienine; N-(13Z-docosenoyl)-4E,6E-tetradecasphingadienine-1-phosphoethanolamine; N-(13Z-docosenoyl)-hexadecasphing-4-enine; N-(13Z-docosenoyl)-hexadecasphing-4-enine-1-phosphocholine; N-(13Z-docosenoyl)-hexadecasphing-4-enine-1-phosphoethanolamine; N-(13Z-docosenoyl)-sphing-4-enine-1-phosphocholine; N-(13Z-docosenoyl)-tetradecasphing-4-enine; N-(13Z-docosenoyl)-tetradecasphing-4-enine-1-phosphoethanolamine; N-(15-hydroperoxy-2R-hydroxy-16E-docosenoyl)-1-beta-glucopyranosyl-4R-hydroxy-13S,14R-methylene-eicosasphingenine; N-(15-oxo-2R-hydroxy-16E-docosenoyl)-1-beta-glucopyranosyl-4R-hydroxy-13S,14R-methylene-eicosasphingenine; N-(15Z-tetracosenoyl)-1-beta-(2′,3′,4′,6′-tetra-O-acetyl-galactosyl)-3-O-acetyl-sphing-4-enine; N-(15Z-tetracosenoyl)-1-beta-(3′-sulfo)-glucosyl-sphing-4-enine; N-(15Z-tetracosenoyl)-1-beta-glucosyl-sphing-4-enine; N-(15Z-tetracosenoyl)-1-beta-glucosyl-sphinganine; N-(15Z-tetracosenoyl)-1-beta-glucosyl-tetradecasphing-4-enine; N-(15Z-tetracosenoyl)-1-beta-lactosyl-sphinganine; N-(15Z-tetracosenoyl)-1-b-lactosyl-sphing-4-enine; N-(15Z-tetracosenoyl)-1-deoxysphing-4-enine; N-(15Z-tetracosenoyl)-1-deoxysphinganine; N-(15Z-tetracosenoyl)-4E,14Z-sphingadienine-1-phosphocholine; N-(15Z-tetracosenoyl)-4E,6E-hexadecasphingadienine-1-phosphoethanolamine; N-(15Z-tetracosenoyl)-4E,6E-tetradecasphingadienine; N-(15Z-tetracosenoyl)-4E,6E-tetradecasphingadienine-1-phosphoethanolamine; N-(15Z-tetracosenoyl)-eicosasphinganine-1-phosphocholine; N-(15Z-tetracosenoyl)-heptadecasphing-4-enine-1-phosphocholine; N-(15Z-tetracosenoyl)-hexadecasphing-4-enine-1-phosphocholine; N-(15Z-tetracosenoyl)-hexadecasphing-4-enine-1-phosphoethanolamine; N-(15Z-tetracosenoyl)-nonadecasphing-4-enine-1-phosphocholine; N-(15Z-tetracosenoyl)-nonadecasphinganine-1-phosphocholine; N-(15Z-tetracosenoyl)-sphing-4-enine; N-(15Z-tetracosenoyl)-sphing-4-enine-1-phosphate; N-(15Z-tetracosenoyl)-sphing-4-enine-1-phosphocholine; N-(15Z-tetracosenoyl)-sphinganine; N-(15Z-tetracosenoyl)-sphinganine-1-phosphocholine; N-(15Z-tetracosenoyl)-tetradecasphing-4-enine; N-(15Z-tetracosenoyl)-tetradecasphing-4-enine-1-phosphoethanolamine; N-(16-hydroperoxy-2R-hydroxy-14E-docosenoyl)-1-beta-glucopyranosyl-4R-hydroxy-13S,14R-methylene-eicosasphingenine; N-(16-hydroperoxy-2R-hydroxy-17E-tricosenoyl)-1-beta-glucopyranosyl-4R-hydroxy-11Z-eicosasphingenine; N-(16-hydroperoxy-2R-hydroxy-17E-tricosenoyl)-1-beta-glucopyranosyl-4R-hydroxy-13Z-eicosasphingenine; N-(16-oxo-2R-hydroxy-14E-docosenoyl)-1-beta-glucopyranosyl-4R-hydroxy-13S,14R-methylene-eicosasphingenine; N-(16-oxo-2R-hydroxy-17E-tricosenoyl)-1-beta-glucopyranosyl-4R-hydroxy-11Z-eicosasphingenine; N-(16-oxo-2R-hydroxy-17E-tricosenoyl)-1-beta-glucopyranosyl-4R-hydroxy-13Z-eicosasphingenine; N-(17-hydroperoxy-2R-hydroxy-15E-tricosenoyl)-1-beta-glucopyranosyl-4R-hydroxy-11Z-eicosasphingenine; N-(17-hydroperoxy-2R-hydroxy-15E-tricosenoyl)-1-beta-glucopyranosyl-4R-hydroxy-13Z-eicosasphingenine; N-(17-oxo-2R-hydroxy-15E-tricosenoyl)-1-beta-glucopyranosyl-4R-hydroxy-11Z-eicosasphingenine; N-(17-oxo-2R-hydroxy-15E-tricosenoyl)-1-beta-glucopyranosyl-4R-hydroxy-13Z-eicosasphingenine; N-(17Z-hexacosenoyl)-1-beta-galactosyl-sphinganine; N-(17Z-hexacosenoyl)-1-beta-glucosyl-sphing-4-enine; N-(17Z-hexacosenoyl)-1-beta-glucosyl-sphinganine; N-(17Z-hexacosenoyl)-1-beta-lactosyl-sphinganine; N-(17Z-hexacosenoyl)-1-b-lactosyl-sphing-4-enine; N-(17Z-hexacosenoyl)-1-deoxysphing-4-enine; N-(17Z-hexacosenoyl)-1-deoxysphinganine; N-(17Z-hexacosenoyl)-heptadecasphing-4-enine-1-phosphocholine; N-(17Z-hexacosenoyl)-sphing-4-enine; N-(17Z-hexacosenoyl)-sphing-4-enine-1-phosphate; N-(17Z-hexacosenoyl)-sphing-4-enine-1-phosphocholine; N-(17Z-hexacosenoyl)-sphinganine; N-(17Z-hexacosenoyl)-sphinganine-1-phosphocholine; N-(2,3-dihydroxy-17Z-tetracosenoyl)-1-beta-glucosyl-4R-hydroxy-sphinganine; N-(24-(9Z,12Z-octadecadienoyloxy)-tetracosanoyl)-docosasphing-4E-enine; N-(24-hydroxytetracosanoyl)-sphinganine; N-(26-(9Z,12Z-octadecadienoyloxy)-hexacosanoyl)-6R-hydroxy-sphing-4E-enine; N-(26-(9Z,12Z-octadecadienoyloxy)-hexacosanoyl)-docosasphing-4E-enine; N-(26-(9Z,12Z-octadecadienoyloxy)-hexacosanoyl)-eicosasphing-4E-enine; N-(26-(9Z,12Z-octadecadienoyloxy)-hexacosanoyl)-heneicosasphing-4E-enine; N-(26-(9Z,12Z-octadecadienoyloxy)-hexacosanoyl)-sphing-4E-enine; N-(26-(9Z,12Z-octadecadienoyloxy)-hexacosanoyl)-sphinganine; N-(26-hydroxy-hexacosanoyl)-sphing-4-enine; N-(26-hydroxyhexacosanoyl)-sphinganine; N-(27-(9Z,12Z-octadecadienoyloxy)-heptacosanoyl)-6R-hydroxy-heneicosasphing-4E-enine; N-(27-(9Z,12Z-octadecadienoyloxy)-heptacosanoyl)-6R-hydroxy-sphing-4E-enine; N-(27-(9Z,12Z-octadecadienoyloxy)-heptacosanoyl)-docosasphing-4E-enine; N-(27-(9Z,12Z-octadecadienoyloxy)-heptacosanoyl)-eicosasphing-4E-enine; N-(27-(9Z,12Z-octadecadienoyloxy)-heptacosanoyl)-heneicosasphing-4E-enine; N-(27-(9Z,12Z-octadecadienoyloxy)-heptacosanoyl)-heneicosasphinganine; N-(27-(9Z,12Z-octadecadienoyloxy)-heptacosanoyl)-sphing-4E-enine; N-(27-hydroxy-heptacosanoyl)-sphing-4-enine; N-(28-(9Z,12Z-octadecadienoyloxy)-octacosanoyl)-4R-hydroxy-docosasphinganine; N-(28-(9Z,12Z-octadecadienoyloxy)-octacosanoyl)-4R-hydroxy-eicosasphinganine; N-(28-(9Z,12Z-octadecadienoyloxy)-octacosanoyl)-4R-hydroxy-heneicosasphinganine; N-(28-(9Z,12Z-octadecadienoyloxy)-octacosanoyl)-4R-hydroxysphinganine; N-(28-(9Z,12Z-octadecadienoyloxy)-octacosanoyl)-6R-hydroxy-docosasphing-4E-enine; N-(28-(9Z,12Z-octadecadienoyloxy)-octacosanoyl)-6R-hydroxy-eicosasphing-4E-enine; N-(28-(9Z,12Z-octadecadienoyloxy)-octacosanoyl)-6R-hydroxy-heneicosasphing-4E-enine; N-(28-(9Z,12Z-octadecadienoyloxy)-octacosanoyl)-6R-hydroxy-heptadecasphing-4E-enine; N-(28-(9Z,12Z-octadecadienoyloxy)-octacosanoyl)-6R-hydroxy-sphing-4E-enine; N-(28-(9Z,12Z-octadecadienoyloxy)-octacosanoyl)-docosasphing-4E-enine; N-(28-(9Z,12Z-octadecadienoyloxy)-octacosanoyl)-docosasphinganine; N-(28-(9Z,12Z-octadecadienoyloxy)-octacosanoyl)-eicosasphing-4E-enine; N-(28-(9Z,12Z-octadecadienoyloxy)-octacosanoyl)-eicosasphinganine; N-(28-(9Z,12Z-octadecadienoyloxy)-octacosanoyl)-heneicosasphing-4E-enine; N-(28-(9Z,12Z-octadecadienoyloxy)-octacosanoyl)-heneicosasphinganine; N-(28-(9Z,12Z-octadecadienoyloxy)-octacosanoyl)-heptadecasphing-4E-enine; N-(28-(9Z,12Z-octadecadienoyloxy)-octacosanoyl)-nonadecasphing-4E-enine; N-(28-(9Z,12Z-octadecadienoyloxy)-octacosanoyl)-nonadecasphinganine; N-(28-(9Z,12Z-octadecadienoyloxy)-octacosanoyl)-sphing-4E-enine; N-(28-(9Z,12Z-octadecadienoyloxy)-octacosanoyl)-sphinganine; N-(28-(9Z,12Z-octadecadienoyloxy)-octacosanoyl)-triacosasphinganine; N-(28-hydroxy-octacosanoyl)-4R-hydroxysphinganine; N-(28-hydroxy-octacosanoyl)-6R-hydroxy-sphing-4E-enine; N-(28-hydroxy-octacosanoyl)-sphing-4-enine; N-(28-hydroxy-octacosanoyl)-sphinganine; N-(29-(9Z,12Z-octadecadienoyloxy)-nonacosanoyl)-4R-hydroxy-docosasphinganine; N-(29-(9Z,12Z-octadecadienoyloxy)-nonacosanoyl)-4R-hydroxy-eicosasphinganine; N-(29-(9Z,12Z-octadecadienoyloxy)-nonacosanoyl)-4R-hydroxy-eicosasphinganine; N-(29-(9Z,12Z-octadecadienoyloxy)-nonacosanoyl)-4R-hydroxysphinganine; N-(29-(9Z,12Z-octadecadienoyloxy)-nonacosanoyl)-6-hydroxy-heptadecasphing-4E-enine; N-(29-(9Z,12Z-octadecadienoyloxy)-nonacosanoyl)-6R-hydroxy-docosasphing-4E-enine; N-(29-(9Z,12Z-octadecadienoyloxy)-nonacosanoyl)-6R-hydroxy-eicosasphing-4E-enine; N-(29-(9Z,12Z-octadecadienoyloxy)-nonacosanoyl)-6R-hydroxy-heneicosasphing-4E-enine; N-(29-(9Z,12Z-octadecadienoyloxy)-nonacosanoyl)-6R-hydroxy-nonadecasphing-4E-enine; N-(29-(9Z,12Z-octadecadienoyloxy)-nonacosanoyl)-6R-hydroxy-sphing-4E-enine; N-(29-(9Z,12Z-octadecadienoyloxy)-nonacosanoyl)-docosasphing-4E-enine; N-(29-(9Z,12Z-octadecadienoyloxy)-nonacosanoyl)-docosasphinganine; N-(29-(9Z,12Z-octadecadienoyloxy)-nonacosanoyl)-eicosasphing-4E-enine; N-(29-(9Z,12Z-octadecadienoyloxy)-nonacosanoyl)-eicosasphinganine; N-(29-(9Z,12Z-octadecadienoyloxy)-nonacosanoyl)-heneicosasphing-4E-enine; N-(29-(9Z,12Z-octadecadienoyloxy)-nonacosanoyl)-heneicosasphinganine; N-(29-(9Z,12Z-octadecadienoyloxy)-nonacosanoyl)-heptadecasphing-4E-enine; N-(29-(9Z,12Z-octadecadienoyloxy)-nonacosanoyl)-hexadecasphing-4E-enine; N-(29-(9Z,12Z-octadecadienoyloxy)-nonacosanoyl)-nonadecasphing-4E-enine; N-(29-(9Z,12Z-octadecadienoyloxy)-nonacosanoyl)-nonadecasphinganine; N-(29-(9Z,12Z-octadecadienoyloxy)-nonacosanoyl)-sphing-4E-enine; N-(29-(9Z,12Z-octadecadienoyloxy)-nonacosanoyl)-sphinganine; N-(29-(9Z,12Z-octadecadienoyloxy)-nonacosanoyl)-triacosasphinganine; N-(29-hydroxy-nonacosanoyl)-4R-hydroxysphinganine; N-(29-hydroxy-nonacosanoyl)-6R-hydroxy-sphing-4E-enine; N-(29-hydroxy-nonacosanoyl)-sphing-4-enine; N-(29-hydroxy-nonacosanoyl)-sphinganine; N-(2-hydroxy-11Z-eicosenoyl)-4E,6E-hexadecasphingadienine; N-(2-hydroxy-11Z-eicosenoyl)-4E,6E-hexadecasphingadienine-1-phosphoethanolamine; N-(2-hydroxy-11Z-eicosenoyl)-4E,6E-tetradecasphingadienine; N-(2-hydroxy-11Z-eicosenoyl)-4E,6E-tetradecasphingadienine-1-phosphoethanolamine; N-(2-hydroxy-11Z-eicosenoyl)-hexadecasphing-4-enine-1-phosphoethanolamine; N-(2-hydroxy-11Z-eicosenoyl)-tetradecasphing-4-enine; N-(2-hydroxy-11Z-eicosenoyl)-tetradecasphing-4-enine-1-phosphoethanolamine; N-(2-hydroxy-13Z-docosenoyl)-1-beta-glucosyl-tetradecasphing-4-enine; N-(2-hydroxy-13Z-docosenoyl)-4E,6E-hexadecasphingadienine; N-(2-hydroxy-13Z-docosenoyl)-4E,6E-hexadecasphingadienine-1-phosphoethanolamine; N-(2-hydroxy-13Z-docosenoyl)-4E,6E-tetradecasphingadienine; N-(2-hydroxy-13Z-docosenoyl)-4E,6E-tetradecasphingadienine-1-phosphoethanolamine; N-(2-hydroxy-13Z-docosenoyl)-hexadecasphing-4-enine-1-phosphoethanolamine; N-(2-hydroxy-13Z-docosenoyl)-tetradecasphing-4-enine; N-(2-hydroxy-13Z-docosenoyl)-tetradecasphing-4-enine-1-phosphoethanolamine; N-(2-hydroxy-15Z-tetracosenoyl)-1-beta-(3′-sulfo)-glucosyl-sphing-4-enine; N-(2-hydroxy-15Z-tetracosenoyl)-1-beta-glucosyl-tetradecasphing-4-enine; N-(2-hydroxy-15Z-tetracosenoyl)-4E,6E-hexadecasphingadienine-1-phosphoethanolamine; N-(2-hydroxy-15Z-tetracosenoyl)-4E,6E-tetradecasphingadienine-1-phosphoethanolamine; N-(2-hydroxy-15Z-tetracosenoyl)-tetradecasphing-4-enine-1-phosphoethanolamine; N-(2-hydroxy-19-methyleicosanoyl)-1-beta-glucosyl-15-methyl-4E-hexadecasphingenine; N-(2-hydroxy-20-methylheneicosanoyl)-1-beta-glucosyl-15-methyl-4E-hexadecasphingenine; N-(2-hydroxy-21-methyldocosanoyl)-1-beta-glucosyl-15-methyl-4E-hexadecasphingenine; N-(2-hydroxy-22-methyltricosanoyl)-1-beta-glucosyl-15-methyl-4E-hexadecasphingenine; N-(2-hydroxy-23-methyltetracosanoyl)-1-beta-glucosyl-15-methyl-4E-hexadecasphingenine; N-(2-hydroxy-24-methylpentacosanoyl)-1-beta-glucosyl-15-methyl-4E-hexadecasphingenine; N-(2-hydroxy-3E-hexadecenoyl)-1-beta-glucosyl-9-methyl-sphinga-4E,8E-dienine; N-(2-hydroxy-3E-octadecenoyl)-1-beta-glucosyl-9-methyl-sphinga-4E,8E-dienine; N-(2-hydroxy-9Z-octadecenoyl)-4E,6E-hexadecasphingadienine; N-(2-hydroxy-9Z-octadecenoyl)-4E,6E-tetradecasphingadienine; N-(2-hydroxy-9Z-octadecenoyl)-hexadecasphing-4-enine; N-(2-hydroxy-9Z-octadecenoyl)-hexadecasphing-4-enine-1-phosphoethanolamine; N-(2-hydroxy-9Z-octadecenoyl)-tetradecasphing-4-enine; N-(2-hydroxy-docosanoyl)-1-beta-(2′,3′,4′,6′-tetra-O-acetyl-galactosyl)-3-O-acetyl-sphing-4-enine; N-(2-hydroxy-docosanoyl)-1-beta-(3′-sulfo)-glucosyl-sphing-4-enine; N-(2-hydroxydocosanoyl)-1-beta-glucosyl-15-methyl-4E-hexadecasphingenine; N-(2-hydroxy-docosanoyl)-1-beta-glucosyl-4E,6E-hexadecasphingadienine; N-(2-hydroxy-docosanoyl)-1-beta-glucosyl-4E,6E-pentadecasphingadienine; N-(2-hydroxy-docosanoyl)-1-beta-glucosyl-4E,6E-tetradecasphingadienine; N-(2-hydroxy-docosanoyl)-1-beta-glucosyl-hexadecasphing-4-enine; N-(2-hydroxy-docosanoyl)-1-beta-glucosyl-tetradecasphing-4-enine; N-(2-hydroxydocosanoyl)-1-glycosyl-4R-hydroxyheptadecasphinganine; N-(2-hydroxy-docosanoyl)-4E,6E-hexadecasphingadienine; N-(2-hydroxy-docosanoyl)-4E,6E-hexadecasphingadienine-1-phosphoethanolamine; N-(2-hydroxy-docosanoyl)-4E,6E-pentadecasphingadienine; N-(2-hydroxy-docosanoyl)-4E,6E-pentadecasphingadienine-1-phosphoethanolamine; N-(2-hydroxy-docosanoyl)-4E,6E-tetradecasphingadienine; N-(2-hydroxy-docosanoyl)-4E,6E-tetradecasphingadienine-1-phosphoethanolamine; N-(2-hydroxydocosanoyl)-4R-hydroxyeicosasphinganine; N-(2-hydroxydocosanoyl)-4R-hydroxyeicosasphinganine-1-O-[D-mannopyranosyl-alpha1-2-myo-inositol-1-phosphate]; N-(2-hydroxydocosanoyl)-4R-hydroxyeicosasphinganine-1-O-[myo-inositol-1-phosphoryl-6-D-mannopyranosyl-alpha1-2-myo-inositol-1-phosphate]; N-(2-hydroxydocosanoyl)-4R-hydroxyeicosasphinganine-1-phospho-(1′-myo-inositol); N-(2-hydroxydocosanoyl)-4R-hydroxyheptadecasphinganine; N-(2-hydroxydocosanoyl)-4R-hydroxysphinganine; N-(2-hydroxydocosanoyl)-4R-hydroxysphinganine-1-O-[D-mannopyranosyl-alpha1-2-myo-inositol-1-phosphate]; N-(2-hydroxydocosanoyl)-4R-hydroxysphinganine-1-O-[myo-inositol-1-phosphoryl-6-D-mannopyranosyl-alpha1-2-myo-inositol-1-phosphate]; N-(2-hydroxydocosanoyl)-4R-hydroxysphinganine-1-phospho-(1′-myo-inositol); N-(2-hydroxydocosanoyl)-6R-hydroxy-sphing-4E-enine; N-(2-hydroxydocosanoyl)-eicosasphinganine; N-(2-hydroxydocosanoyl)-eicosasphinganine-1-O-[D-mannopyranosyl-alpha1-2-myo-inositol-1-phosphate]; N-(2-hydroxydocosanoyl)-eicosasphinganine-1-O-[myo-inositol-1-phosphoryl-6-D-mannopyranosyl-alpha1-2-myo-inositol-1-phosphate]; N-(2-hydroxydocosanoyl)-eicosasphinganine-1-phospho-(1′-myo-inositol); N-(2-hydroxy-docosanoyl)-hexadecasphing-4-enine; N-(2-hydroxy-docosanoyl)-hexadecasphing-4-enine-1-phosphoethanolamine; N-(2-hydroxy-docosanoyl)-pentadecasphing-4-enine-1-phosphoethanolamine; N-(2-hydroxy-docosanoyl)-sphing-4-enine; N-(2-hydroxydocosanoyl)-sphinganine; N-(2-hydroxydocosanoyl)-sphinganine-1-O-[D-mannopyranosyl-alpha1-2-myo-inositol-1-phosphate]; N-(2-hydroxydocosanoyl)-sphinganine-1-O-[myo-inositol-1-phosphoryl-6-D-mannopyranosyl-alpha1-2-myo-inositol-1-phosphate]; N-(2-hydroxydocosanoyl)-sphinganine-1-phospho-(1′-myo-inositol); N-(2-hydroxy-docosanoyl)-tetradecasphing-4-enine; N-(2-hydroxy-docosanoyl)-tetradecasphing-4-enine-1-phosphoethanolamine; N-(2-hydroxydotriacontanoyl)-4R-hydroxysphinganine; N-(2-hydroxy-dotriacontanoyl)-sphing-4-enine; N-(2-hydroxy-eicosanoyl)-1-beta-(3′-sulfo)-glucosyl-sphing-4-enine; N-(2-hydroxyeicosanoyl)-1-beta-glucosyl-15-methyl-4E-hexadecasphingenine; N-(2-hydroxy-eicosanoyl)-1-beta-glucosyl-4E,6E-hexadecasphingadienine; N-(2-hydroxy-eicosanoyl)-1-beta-glucosyl-4E,6E-pentadecasphingadienine; N-(2-hydroxy-eicosanoyl)-1-beta-glucosyl-4E,6E-tetradecasphingadienine; N-(2-hydroxy-eicosanoyl)-1-beta-glucosyl-hexadecasphing-4-enine; N-(2-hydroxy-eicosanoyl)-1-beta-glucosyl-tetradecasphing-4-enine; N-(2-hydroxy-eicosanoyl)-4E,6E-hexadecasphingadienine; N-(2-hydroxy-eicosanoyl)-4E,6E-hexadecasphingadienine-1-phosphoethanolamine; N-(2-hydroxy-eicosanoyl)-4E,6E-pentadecasphingadienine; N-(2-hydroxy-eicosanoyl)-4E,6E-pentadecasphingadienine-1-phosphoethanolamine; N-(2-hydroxy-eicosanoyl)-4E,6E-tetradecasphingadienine; N-(2-hydroxy-eicosanoyl)-4E,6E-tetradecasphingadienine-1-phosphoethanolamine; N-(2-hydroxyeicosanoyl)-4R-hydroxyeicosasphinganine; N-(2-hydroxyeicosanoyl)-4R-hydroxyeicosasphinganine-1-O-[D-mannopyranosyl-alpha1-2-myo-inositol-1-phosphate]; N-(2-hydroxyeicosanoyl)-4R-hydroxyeicosasphinganine-1-O-[myo-inositol-1-phosphoryl-6-D-mannopyranosyl-alpha1-2-myo-inositol-1-phosphate]; N-(2-hydroxyeicosanoyl)-4R-hydroxyeicosasphinganine-1-phospho-(1′-myo-inositol); N-(2-hydroxyeicosanoyl)-4R-hydroxysphinganine; N-(2-hydroxyeicosanoyl)-4R-hydroxysphinganine-1-O-[D-mannopyranosyl-alpha1-2-myo-inositol-1-phosphate]; N-(2-hydroxyeicosanoyl)-4R-hydroxysphinganine-1-O-[myo-inositol-1-phosphoryl-6-D-mannopyranosyl-alpha1-2-myo-inositol-1-phosphate]; N-(2-hydroxyeicosanoyl)-4R-hydroxysphinganine-1-phospho-(1′-myo-inositol); N-(2-hydroxyeicosanoyl)-6R-hydroxy-sphing-4E-enine; N-(2-hydroxyeicosanoyl)-eicosasphinganine; N-(2-hydroxyeicosanoyl)-eicosasphinganine-1-O-[D-mannopyranosyl-alpha1-2-myo-inositol-1-phosphate]; N-(2-hydroxyeicosanoyl)-eicosasphinganine-1-O-[myo-inositol-1-phosphoryl-6-D-mannopyranosyl-alpha1-2-myo-inositol-1-phosphate]; N-(2-hydroxyeicosanoyl)-eicosasphinganine-1-phospho-(1′-myo-inositol); N-(2-hydroxy-eicosanoyl)-hexadecasphing-4-enine; N-(2-hydroxy-eicosanoyl)-hexadecasphing-4-enine-1-phosphoethanolamine; N-(2-hydroxy-eicosanoyl)-pentadecasphing-4-enine-1-phosphoethanolamine; N-(2-hydroxy-eicosanoyl)-sphing-4-enine; N-(2-hydroxyeicosanoyl)-sphinganine; N-(2-hydroxyeicosanoyl)-sphinganine-1-O-[D-mannopyranosyl-alpha1-2-myo-inositol-1-phosphate]; N-(2-hydroxyeicosanoyl)-sphinganine-1-O-[myo-inositol-1-phosphoryl-6-D-mannopyranosyl-alpha1-2-myo-inositol-1-phosphate]; N-(2-hydroxyeicosanoyl)-sphinganine-1-phospho-(1′-myo-inositol); N-(2-hydroxy-eicosanoyl)-tetradecasphing-4-enine; N-(2-hydroxy-eicosanoyl)-tetradecasphing-4-enine-1-phosphoethanolamine; N-(2-hydroxyheneicosanoyl)-1-beta-glucosyl-15-methyl-4E-hexadecasphingenine; N-(2-hydroxyheneicosanoyl)-4R-hydroxysphinganine; N-(2-hydroxyheneicosanoyl)-6R-hydroxy-sphing-4E-enine; N-(2-hydroxy-heneicosanoyl)-sphing-4-enine; N-(2-hydroxyhentriacontanoyl)-4R-hydroxysphinganine; N-(2-hydroxy-hentriacontanoyl)-sphing-4-enine; N-(2-hydroxy-heptacosanoyl)-4E,14Z-sphingadienine; N-(2-hydroxyheptacosanoyl)-4R-hydroxysphinganine; N-(2-hydroxyheptacosanoyl)-6R-hydroxy-sphing-4E-enine; N-(2-hydroxy-heptacosanoyl)-sphing-4-enine; N-(2-hydroxyheptacosanoyl)-sphinganine; N-(2-hydroxyheptadecanoyl)-4R-hydroxysphinganine; N-(2-hydroxyheptadecanoyl)-6R-hydroxy-sphing-4E-enine; N-(2-hydroxy-heptadecanoyl)-sphing-4-enine; N-(2-hydroxyhexacosanoyl)-1-beta-glucosyl-15-methyl-4E-hexadecasphingenine; N-(2-hydroxy-hexacosanoyl)-4E,14Z-sphingadienine; N-(2-hydroxyhexacosanoyl)-4R-hydroxyeicosasphinganine; N-(2-hydroxyhexacosanoyl)-4R-hydroxyeicosasphinganine-1-O-[D-mannopyranosyl-alpha1-2-myo-inositol-1-phosphate]; N-(2-hydroxyhexacosanoyl)-4R-hydroxyeicosasphinganine-1-O-[myo-inositol-1-phosphoryl-6-D-mannopyranosyl-alpha1-2-myo-inositol-1-phosphate]; N-(2-hydroxyhexacosanoyl)-4R-hydroxyeicosasphinganine-1-phospho-(1′-myo-inositol); N-(2-hydroxyhexacosanoyl)-4R-hydroxysphinganine; N-(2-hydroxy-hexacosanoyl)-4R-hydroxysphinganine-1-O-[inositol-1-phosphoryl-6-mannosyl-alpha1-2-inositol-1-phosphate]; N-(2-hydroxy-hexacosanoyl)-4R-hydroxysphinganine-1-O-[mannosyl-alpha1-2-inositol-1-phosphate]; N-(2-hydroxyhexacosanoyl)-4R-hydroxysphinganine-1-phospho-(1′-myo-inositol); N-(2-hydroxyhexacosanoyl)-6R-hydroxy-sphing-4E-enine; N-(2-hydroxyhexacosanoyl)-eicosasphinganine; N-(2-hydroxyhexacosanoyl)-eicosasphinganine-1-O-[D-mannopyranosyl-alpha1-2-myo-inositol-1-phosphate]; N-(2-hydroxyhexacosanoyl)-eicosasphinganine-1-O-[myo-inositol-1-phosphoryl-6-D-mannopyranosyl-alpha1-2-myo-inositol-1-phosphate]; N-(2-hydroxyhexacosanoyl)-eicosasphinganine-1-phospho-(1′-myo-inositol); N-(2-hydroxy-hexacosanoyl)-sphing-4-enine; N-(2-hydroxyhexacosanoyl)-sphinganine; N-(2-hydroxyhexacosanoyl)-sphinganine-1-O-[D-mannopyranosyl-alpha1-2-myo-inositol-1-phosphate]; N-(2-hydroxyhexacosanoyl)-sphinganine-1-O-[myo-inositol-1-phosphoryl-6-D-mannopyranosyl-alpha1-2-myo-inositol-1-phosphate]; N-(2-hydroxyhexacosanoyl)-sphinganine-1-phospho-(1′-myo-inositol); N-(2-hydroxy-hexacosanoyl)-tetradecasphing-4-enine; N-(2-hydroxy-hexadecanoyl)-1-beta-(3′-sulfo)-glucosyl-sphing-4-enine; N-(2-hydroxy-hexadecanoyl)-1-beta-glucosyl-9-methyl-sphinga-4E,8E-dienine; N-(2-hydroxy-hexadecanoyl)-4E,14Z-sphingadienine; N-(2-hydroxy-hexadecanoyl)-4E,6E-hexadecasphingadienine-1-phosphoethanolamine; N-(2-hydroxy-hexadecanoyl)-4E,6E-tetradecasphingadienine-1-phosphoethanolamine; N-(2-hydroxy-hexadecanoyl)-4R-hydroxy-11E-sphingenine; N-(2-hydroxyhexadecanoyl)-4R-hydroxyeicosasphinganine; N-(2-hydroxyhexadecanoyl)-4R-hydroxyeicosasphinganine-1-O-[D-mannopyranosyl-alpha1-2-myo-inositol-1-phosphate]; N-(2-hydroxyhexadecanoyl)-4R-hydroxyeicosasphinganine-1-O-[myo-inositol-1-phosphoryl-6-D-mannopyranosyl-alpha1-2-myo-inositol-1-phosphate]; N-(2-hydroxyhexadecanoyl)-4R-hydroxyeicosasphinganine-1-phospho-(1′-myo-inositol); N-(2-hydroxyhexadecanoyl)-4R-hydroxysphinganine; N-(2-hydroxyhexadecanoyl)-4R-hydroxysphinganine-1-O-[D-mannopyranosyl-alpha1-2-myo-inositol-1-phosphate]; N-(2-hydroxyhexadecanoyl)-4R-hydroxysphinganine-1-O-[myo-inositol-1-phosphoryl-6-D-mannopyranosyl-alpha1-2-myo-inositol-1-phosphate]; N-(2-hydroxyhexadecanoyl)-4R-hydroxysphinganine-1-phospho-(1′-myo-inositol); N-(2-hydroxyhexadecanoyl)-6R-hydroxy-sphing-4E-enine; N-(2-hydroxyhexadecanoyl)-eicosasphinganine; N-(2-hydroxyhexadecanoyl)-eicosasphinganine-1-O-[D-mannopyranosyl-alpha1-2-myo-inositol-1-phosphate]; N-(2-hydroxyhexadecanoyl)-eicosasphinganine-1-O-[myo-inositol-1-phosphoryl-6-D-mannopyranosyl-alpha1-2-myo-inositol-1-phosphate]; N-(2-hydroxyhexadecanoyl)-eicosasphinganine-1-phospho-(1′-myo-inositol); N-(2-hydroxy-hexadecanoyl)-sphing-4-enine; N-(2-hydroxyhexadecanoyl)-sphing-4-enine-1-phosphate; N-(2-hydroxyhexadecanoyl)-sphing-4-enine-1-phosphocholine; N-(2-hydroxyhexadecanoyl)-sphinganine; N-(2-hydroxyhexadecanoyl)-sphinganine-1-O-[D-mannopyranosyl-alpha1-2-myo-inositol-1-phosphate]; N-(2-hydroxyhexadecanoyl)-sphinganine-1-O-[myo-inositol-1-phosphoryl-6-D-mannopyranosyl-alpha1-2-myo-inositol-1-phosphate]; N-(2-hydroxyhexadecanoyl)-sphinganine-1-phospho-(1′-myo-inositol); N-(2-hydroxynonacosanoyl)-4R-hydroxysphinganine; N-(2-hydroxynonacosanoyl)-6R-hydroxy-sphing-4E-enine; N-(2-hydroxy-nonacosanoyl)-sphing-4-enine; N-(2-hydroxynonadecanoyl)-4R-hydroxysphinganine; N-(2-hydroxynonadecanoyl)-6R-hydroxy-sphing-4E-enine; N-(2-hydroxy-nonadecanoyl)-sphing-4-enine; N-(2-hydroxy-octacosanoyl)-4E,14Z-sphingadienine; N-(2-hydroxyoctacosanoyl)-4R-hydroxysphinganine; N-(2-hydroxyoctacosanoyl)-6R-hydroxy-sphing-4E-enine; N-(2-hydroxy-octacosanoyl)-sphing-4-enine; N-(2-hydroxy-octadecanoyl)-1-beta-(3′-sulfo)-glucosyl-sphing-4-enine; N-(2-hydroxy-octadecanoyl)-1-beta-glucosyl-4E,6E-hexadecasphingadienine; N-(2-hydroxy-octadecanoyl)-1-beta-glucosyl-4E,6E-tetradecasphingadienine; N-(2-hydroxy-octadecanoyl)-1-beta-glucosyl-9-methyl-sphinga-4E,8E-dienine; N-(2-hydroxy-octadecanoyl)-4E,14Z-sphingadienine; N-(2-hydroxy-octadecanoyl)-4E,6E-hexadecasphingadienine; N-(2-hydroxy-octadecanoyl)-4E,6E-hexadecasphingadienine-1-phosphoethanolamine; N-(2-hydroxy-octadecanoyl)-4E,6E-pentadecasphingadienine-1-phosphoethanolamine; N-(2-hydroxy-octadecanoyl)-4E,6E-tetradecasphingadienine; N-(2-hydroxy-octadecanoyl)-4E,6E-tetradecasphingadienine-1-phosphoethanolamine; N-(2-hydroxyoctadecanoyl)-4R-hydroxyeicosasphinganine; N-(2-hydroxyoctadecanoyl)-4R-hydroxyeicosasphinganine-1-O-[D-mannopyranosyl-alpha1-2-myo-inositol-1-phosphate]; N-(2-hydroxyoctadecanoyl)-4R-hydroxyeicosasphinganine-1-O-[myo-inositol-1-phosphoryl-6-D-mannopyranosyl-alpha1-2-myo-inositol-1-phosphate]; N-(2-hydroxyoctadecanoyl)-4R-hydroxyeicosasphinganine-1-phospho-(1′-myo-inositol); N-(2-hydroxyoctadecanoyl)-4R-hydroxysphinganine; N-(2-hydroxyoctadecanoyl)-4R-hydroxysphinganine-1-O-[D-mannopyranosyl-alpha1-2-myo-inositol-1-phosphate]; N-(2-hydroxyoctadecanoyl)-4R-hydroxysphinganine-1-O-[myo-inositol-1-phosphoryl-6-D-mannopyranosyl-alpha1-2-myo-inositol-1-phosphate]; N-(2-hydroxyoctadecanoyl)-4R-hydroxysphinganine-1-phospho-(1′-myo-inositol); N-(2-hydroxyoctadecanoyl)-6R-hydroxy-sphing-4E-enine; N-(2-hydroxyoctadecanoyl)-eicosasphinganine; N-(2-hydroxyoctadecanoyl)-eicosasphinganine-1-O-[D-mannopyranosyl-alpha1-2-myo-inositol-1-phosphate]; N-(2-hydroxyoctadecanoyl)-eicosasphinganine-1-O-[myo-inositol-1-phosphoryl-6-D-mannopyranosyl-alpha1-2-myo-inositol-1-phosphate]; N-(2-hydroxyoctadecanoyl)-eicosasphinganine-1-phospho-(1′-myo-inositol); N-(2-hydroxy-octadecanoyl)-hexadecasphing-4-enine-1-phosphoethanolamine; N-(2-hydroxy-octadecanoyl)-sphing-4-enine; N-(2-hydroxyoctadecanoyl)-sphinganine; N-(2-hydroxyoctadecanoyl)-sphinganine-1-O-[D-mannopyranosyl-alpha1-2-myo-inositol-1-phosphate]; N-(2-hydroxyoctadecanoyl)-sphinganine-1-O-[myo-inositol-1-phosphoryl-6-D-mannopyranosyl-alpha1-2-myo-inositol-1-phosphate]; N-(2-hydroxyoctadecanoyl)-sphinganine-1-phospho-(1′-myo-inositol); N-(2-hydroxy-octadecanoyl)-tetradecasphing-4-enine-1-phosphoethanolamine; N-(2-hydroxypentacosanoyl)-1-beta-glucosyl-15-methyl-4E-hexadecasphingenine; N-(2-hydroxy-pentacosanoyl)-4E,14Z-sphingadienine; N-(2-hydroxypentacosanoyl)-4R-hydroxysphinganine; N-(2-hydroxypentacosanoyl)-6R-hydroxy-sphing-4E-enine; N-(2-hydroxy-pentacosanoyl)-sphing-4-enine; N-(2-hydroxypentacosanoyl)-sphinganine; N-(2-hydroxypentadecanoyl)-6R-hydroxy-sphing-4E-enine; N-(2-hydroxy-pentadecanoyl)-sphing-4-enine; N-(2-hydroxypentatriacontanoyl)-4R-hydroxysphinganine; N-(2-hydroxy-tetracosanoyl)-1-beta-(2′,3′,4′,6′-tetra-O-acetyl-galactosyl)-3-O-acetyl-sphing-4-enine; N-(2-hydroxy-tetracosanoyl)-1-beta-(3′-sulfo)-glucosyl-sphing-4-enine; N-(2-hydroxytetracosanoyl)-1-beta-glucosyl-15-methyl-4E-hexadecasphingenine; N-(2-hydroxy-tetracosanoyl)-1-beta-glucosyl-4E,6E-hexadecasphingadienine; N-(2-hydroxy-tetracosanoyl)-1-beta-glucosyl-4E,6E-tetradecasphingadienine; N-(2-hydroxy-tetracosanoyl)-1-beta-glucosyl-tetradecasphing-4-enine; N-(2-hydroxytetracosanoyl)-1-glycosyl-4R-hydroxyheptadecasphinganine; N-(2-hydroxy-tetracosanoyl)-4E,14Z-sphingadienine; N-(2-hydroxy-tetracosanoyl)-4E,6E-hexadecasphingadienine; N-(2-hydroxy-tetracosanoyl)-4E,6E-hexadecasphingadienine-1-phosphoethanolamine; N-(2-hydroxy-tetracosanoyl)-4E,6E-pentadecasphingadienine-1-phosphoethanolamine; N-(2-hydroxy-tetracosanoyl)-4E,6E-tetradecasphingadienine; N-(2-hydroxy-tetracosanoyl)-4E,6E-tetradecasphingadienine-1-phosphoethanolamine; N-(2-hydroxytetracosanoyl)-4R-hydroxyeicosasphinganine; N-(2-hydroxytetracosanoyl)-4R-hydroxyeicosasphinganine-1-O-[D-mannopyranosyl-alpha1-2-myo-inositol-1-phosphate]; N-(2-hydroxytetracosanoyl)-4R-hydroxyeicosasphinganine-1-O-[myo-inositol-1-phosphoryl-6-D-mannopyranosyl-alpha1-2-myo-inositol-1-phosphate]; N-(2-hydroxytetracosanoyl)-4R-hydroxyeicosasphinganine-1-phospho-(1′-myo-inositol); N-(2-hydroxytetracosanoyl)-4R-hydroxyheptadecasphinganine; N-(2-hydroxy-tetracosanoyl)-4R-hydroxysphing-8E-enine-1-O-[2-amino-D-glucopyranosyl(alpha1-4)-D-glucopyranosyl(alpha1-2)-myo-inositol-1-phosphate]; N-(2-hydroxytetracosanoyl)-4R-hydroxysphinganine; N-(2-hydroxytetracosanoyl)-4R-hydroxysphinganine-1-O-[D-mannopyranosyl-alpha1-2-myo-inositol-1-phosphate]; N-(2-hydroxytetracosanoyl)-4R-hydroxysphinganine-1-O-[myo-inositol-1-phosphoryl-6-D-mannopyranosyl-alpha1-2-myo-inositol-1-phosphate]; N-(2-hydroxytetracosanoyl)-4R-hydroxysphinganine-1-phospho-(1′-myo-inositol); N-(2-hydroxytetracosanoyl)-6R-hydroxy-sphing-4E-enine; N-(2-hydroxytetracosanoyl)-eicosasphinganine; N-(2-hydroxytetracosanoyl)-eicosasphinganine-1-O-[D-mannopyranosyl-alpha1-2-myo-inositol-1-phosphate]; N-(2-hydroxytetracosanoyl)-eicosasphinganine-1-O-[myo-inositol-1-phosphoryl-6-D-mannopyranosyl-alpha1-2-myo-inositol-1-phosphate]; N-(2-hydroxytetracosanoyl)-eicosasphinganine-1-phospho-(1′-myo-inositol); N-(2-hydroxy-tetracosanoyl)-hexadecasphing-4-enine; N-(2-hydroxy-tetracosanoyl)-hexadecasphing-4-enine-1-phosphoethanolamine; N-(2-hydroxy-tetracosanoyl)-sphing-4-enine; N-(2-hydroxytetracosanoyl)-sphinganine; N-(2-hydroxytetracosanoyl)-sphinganine-1-O-[D-mannopyranosyl-alpha1-2-myo-inositol-1-phosphate]; N-(2-hydroxytetracosanoyl)-sphinganine-1-O-[myo-inositol-1-phosphoryl-6-D-mannopyranosyl-alpha1-2-myo-inositol-1-phosphate]; N-(2-hydroxytetracosanoyl)-sphinganine-1-phospho-(1′-myo-inositol); N-(2-hydroxy-tetracosanoyl)-tetradecasphing-4-enine; N-(2-hydroxy-tetracosanoyl)-tetradecasphing-4-enine-1-phosphoethanolamine; N-(2-hydroxytetradecanoyl)-6R-hydroxy-sphing-4E-enine; N-(2-hydroxy-tetradecanoyl)-sphing-4-enine; N-(2-hydroxytetratriacontanoyl)-4R-hydroxysphinganine; N-(2-hydroxytriacontanoyl)-4R-hydroxysphinganine; N-(2-hydroxytriacontanoyl)-6R-hydroxy-sphing-4E-enine; N-(2-hydroxy-triacontanoyl)-sphing-4-enine; N-(2-hydroxytricosanoyl)-1-beta-glucosyl-15-methyl-4E-hexadecasphingenine; N-(2-hydroxytricosanoyl)-4R-hydroxysphinganine; N-(2-hydroxytricosanoyl)-6R-hydroxy-sphing-4E-enine; N-(2-hydroxy-tricosanoyl)-sphing-4-enine; N-(2-hydroxytritriacontanoyl)-4R-hydroxysphinganine; N-(2R,15-dihydroxy-16E-docosenoyl)-1-beta-glucopyranosyl-4R-hydroxy-13S,14R-methylene-eicosasphingenine; N-(2R,16-dihydroxy-14E-docosenoyl)-1-beta-glucopyranosyl-4R-hydroxy-13S,14R-methylene-eicosasphingenine; N-(2R,16-dihydroxy-17E-tricosenoyl)-1-beta-glucopyranosyl-4R-hydroxy-11Z-eicosasphingenine; N-(2R,16-dihydroxy-17E-tricosenoyl)-1-beta-glucopyranosyl-4R-hydroxy-13Z-eicosasphingenine; N-(2R,17-dihydroxy-15E-tricosenoyl)-1-beta-glucopyranosyl-4R-hydroxy-11Z-eicosasphingenine; N-(2R,17-dihydroxy-15E-tricosenoyl)-1-beta-glucopyranosyl-4R-hydroxy-13Z-eicosasphingenine; N-(2R,3-dihydroxyhexacosanoyl)-4R-hydroxy-8E-octadecasphingenine; N-(2R-hydroxy-heneicosanoyl)-4R-hydroxy-nonadecasphinganine; N-(2R-hydroxy-pentadecanoyl)-4R-hydroxy-heptadecasphinganine; N-(2R-hydroxy-15-methyl-3E-octadecenoyl)-4E,10E,12E-sphingatrienine; N-(2R-hydroxy-16Z-pentacosenoyl)-1-beta-D-glucosyl-sphing-4E,8E,10E-trienine; N-(2R-hydroxy-17Z-hexacosenoyl)-1-beta-D-glucosyl-9-methyl-sphing-4E,8E,10E-trienine; N-(2R-hydroxy-17Z-hexacosenoyl)-1-beta-D-glucosyl-sphing-4E,8E,10E-trienine; N-(2R-hydroxy-17Z-tetracosenoyl)-1-beta-glucosyl-4R-hydroxy-sphinganine; N-(2R-hydroxy-17Z-tetracosenoyl)-1-beta-glucosyl-9-methyl-sphing-4E,8E-dienine; N-(2R-hydroxy-20Z-heptacosenoyl)-1-beta-D-glucosyl-9-methyl-sphing-4E,8E,10E-trienine; N-(2R-hydroxy-21Z-octacosenoyl)-1-beta-D-glucosyl-9-methyl-sphing-4E,8E,10E-trienine; N-(2R-hydroxy-21Z-octacosenoyl)-1-beta-D-glucosyl-sphing-4E,8E,10E-trienine; N-(2R-hydroxy-3E-heneicosenoyl)-1-beta-glucosyl-9-methyl-heptadecasphing-4E,8E-dienine; N-(2R-hydroxy-3E-nonadecenoyl)-1-beta-D-glucopyranosyl-9-methyl-heptadecasphing-4E,8E-dienine; N-(2R-hydroxy-3E-nonadecenoyl)-1-beta-D-glucopyranosyl-9-methyl-pentadecasphing-4E,8E-dienine; N-(2R-hydroxy-3E-nonadecenoyl)-1-beta-D-glucopyranosyl-9-methyl-tetradecasphing-4E,8E-dienine; N-(2R-hydroxy-3E-octadecenoyl)-1-beta-D-galactosyl-9-methyl-sphing-4E,8E-dienine; N-(2R-hydroxy-3E-octadecenoyl)-1-beta-D-glucopyranosyl-hexadecasphing-4E,8Z-dienine; N-(2R-hydroxy-3E-octadecenoyl)-1-beta-glucosyl-10-methyl-sphinga-4E,9E-dienine; N-(2R-hydroxy-7Z-pentadecenoyl)-(6-O-(2E)-3-(3,4-dimethoxyphenyl)prop-2-enoyl)-1-alpha-glucosyl-4E-nonadecasphingenine; N-(2R-hydroxy-7Z-pentadecenoyl)-1-alpha-glucosyl-4E-nonadecasphingenine; N-(2R-hydroxy-9Z-tetracosenoyl)-4R-hydroxysphinganine; N-(2R-hydroxydecanoyl)-4R-hydroxy-8E-hexacosasphingenine; N-(2R-hydroxy-docosanoyl)-1-beta-D-galactosyl-9-methyl-sphing-4E,8E,10E-trienine; N-(2R-hydroxy-docosanoyl)-1-beta-D-galactosyl-sphing-4E,8E,10E-trienine; N-(2R-hydroxy-docosanoyl)-1-beta-D-glucosyl-9-methyl-sphing-4E,8E,10E-trienine; N-(2R-hydroxy-docosanoyl)-1-beta-D-glucosyl-sphing-4E,8E,10E-trienine; N-(2R-hydroxydocosanoyl)-1-beta-glucopyranosyl-4R-hydroxy-14-methyl-pentadecasphinganine; N-(2R-hydroxydocosanoyl)-1-beta-glucopyranosyl-4R-hydroxy-15-methyl-hexadecasphinganine; N-(2R-hydroxydocosanoyl)-1-beta-glucopyranosyl-4R-hydroxy-16-methyl-heptadecasphinganine; N-(2R-hydroxydocosanoyl)-1-beta-glucosyl-4E,8Z-octadecasphingadienine; N-(2R-hydroxydocosanoyl)-1-beta-glucosyl-4R-hydroxy-8Z-octadecasphingenine; N-(2R-hydroxydocosanoyl)-1-beta-glucosyl-8E-octadecasphingenine; N-(2R-hydroxydocosanoyl)-1-beta-glucosyl-8Z-octadecasphingenine; N-(2R-hydroxy-docosanoyl)-1-beta-glucosyl-9-methyl-sphing-4E,8E-dienine; N-(2R-hydroxydocosanoyl)-4R-hydroxy-14-methyl-pentadecasphinganine; N-(2R-hydroxydocosanoyl)-4R-hydroxy-15-methyl-hexadecasphinganine; N-(2R-hydroxydocosanoyl)-4R-hydroxy-16-methyl-heptadecasphinganine; N-(2R-hydroxy-docosanoyl)-4R-hydroxy-docosasphing-6Z-enine; N-(2R-hydroxy-eicosanoyl)-1-beta-D-glucosyl-9-methyl-sphing-4E,8E,10E-trienine; N-(2R-hydroxyeicosanoyl)-1-beta-glucosyl-4E,8Z-octadecasphingadienine; N-(2R-hydroxyeicosanoyl)-1-beta-glucosyl-octadecasphinganine; N-(2R-hydroxyeicosanoyl)-4E,8Z-octadecasphingadienine; N-(2R-hydroxyeicosanoyl)-4R-hydroxy-8Z-octadecasphingenine; N-(2R-hydroxy-heneicosanoyl)-1-beta-D-glucosyl-9-methyl-sphing-4E,8E,10E-trienine; N-(2R-hydroxyheneicosanoyl)-1-beta-glucopyranosyl-4R-hydroxy-14-methyl-pentadecasphinganine; N-(2R-hydroxyheneicosanoyl)-1-beta-glucopyranosyl-4R-hydroxy-15-methyl-hexadecasphinganine; N-(2R-hydroxyheneicosanoyl)-1-beta-glucopyranosyl-4R-hydroxy-16-methyl-heptadecasphinganine; N-(2R-hydroxyheneicosanoyl)-1-beta-glucosyl-4E,8Z-octadecasphingadienine; N-(2R-hydroxyheneicosanoyl)-1-beta-glucosyl-8E-octadecasphingenine; N-(2R-hydroxyheneicosanoyl)-1-beta-glucosyl-8Z-octadecasphingenine; N-(2R-hydroxy-heneicosanoyl)-1-beta-glucosyl-9-methyl-heptadecasphing-4E,8E-dienine; N-(2R-hydroxyheneicosanoyl)-4R-hydroxy-14-methyl-pentadecasphinganine; N-(2R-hydroxyheneicosanoyl)-4R-hydroxy-15-methyl-hexadecasphinganine; N-(2R-hydroxyheneicosanoyl)-4R-hydroxy-16-methyl-heptadecasphinganine; N-(2R-hydroxy-heptadecanoyl)-1-beta-D-glucosyl-9-methyl-sphing-4E,8E,10E-trienine; N-(2R-hydroxyheptadecanoyl)-1-beta-glucosyl-4E,8Z-octadecasphingadienine; N-(2R-hydroxy-heptadecanoyl)-1-beta-glucosyl-9-methyl-sphing-4E,8E-dienine; N-(2R-hydroxyheptadecanoyl)-sphinganine; N-(2R-hydroxyhexacosanoyl)-1-beta-glucosyl-4R-hydroxy-8Z-octadecasphingenine; N-(2R-hydroxyhexacosanoyl)-4R-hydroxy-8E-octadecasphingenine; N-(2R-hydroxyhexadecanoyl)-1-beta-glucosyl-4E,8E-octadecasphingadienine; N-(2R-hydroxyhexadecanoyl)-1-beta-glucosyl-4E,8Z-octadecasphingadienine; N-(2R-hydroxyhexadecanoyl)-1-beta-glucosyl-4E,8Z-sphingadienine; N-(2R-hydroxyhexadecanoyl)-1-beta-glucosyl-4R-hydroxy-8Z-octadecasphingenine; N-(2R-hydroxyhexadecanoyl)-1-beta-glucosyl-5E,12E-sphingdienine; N-(2R-hydroxy-hexadecanoyl)-1-beta-glucosyl-8-hydroxy,9-methylene-sphing-4E-enine; N-(2R-hydroxy-hexadecanoyl)-1-beta-glucosyl-8-hydroxy,9-methyl-sphing-4E-enine; N-(2R-hydroxyhexadecanoyl)-1-beta-glucosyl-8Z-octadecasphingenine; N-(2R-hydroxy-hexadecanoyl)-1-beta-glucosyl-9-hydroxy,9-methyl-sphing-4E,7E-dienine; N-(2R-hydroxy-hexadecanoyl)-1-beta-glucosyl-9-methyl-sphing-4E,8E,10E-trienine; N-(2R-hydroxy-hexadecanoyl)-1-beta-glucosyl-9-methyl-sphing-4E,8E-dienine; N-(2R-hydroxyhexadecanoyl)-1-beta-glucosyl-octadecasphinganine; N-(2R-hydroxyhexadecanoyl)-4R-hydroxy-20E-hexacosasphingenine; N-(2R-hydroxyhexadecanoyl)-8E-octadecasphingenine; N-(2R-hydroxyhexadecanoyl)-8Z-octadecasphingenine; N-(2R-hydroxy-hexadecanoyl)-1-beta-glucosyl-9-methyl-heptadecasphing-4E,8E-dienine; N-(2R-hydroxyicosanoyl)-1-beta-glucosyl-4E,8E-octadecasphingadienine; N-(2R-hydroxyicosanoyl)-1-beta-glucosyl-4R-hydroxy-8Z-octadecasphingenine; N-(2R-hydroxyicosanoyl)-1-beta-glucosyl-8Z-octadecasphingenine; N-(2R-hydroxyicosanoyl)-8Z-octadecasphingenine; N-(2R-hydroxyicosanoyl)-8Z-octadecasphingenine; N-(2R-hydroxy-nonadecanoyl)-1-beta-D-glucopyranosyl-9-methyl-pentadecasphing-4E,8E-dienine; N-(2R-hydroxynonadecanoyl)-1-beta-glucosyl-4E,8Z-octadecasphingadienine; N-(2R-hydroxynonadecanoyl)-4E,8Z-octadecasphingadienine; N-(2R-hydroxy-octadecanoyl)-1-beta-D-galactosyl-9-methyl-sphing-4E,8E-dienine; N-(2R-hydroxy-octadecanoyl)-1-beta-D-glucosyl-9-methyl-sphing-4E,8E,10E-trienine; N-(2R-hydroxy-octadecanoyl)-1-beta-glucosyl-10-methyl-sphinga-4E,9E-dienine; N-(2R-hydroxyoctadecanoyl)-1-beta-glucosyl-4E,8E-octadecasphingadienine; N-(2R-hydroxyoctadecanoyl)-1-beta-glucosyl-4E,8Z-octadecasphingadienine; N-(2R-hydroxyoctadecanoyl)-1-beta-glucosyl-4E,8Z-octadecasphingadienine; N-(2R-hydroxyoctadecanoyl)-1-beta-glucosyl-4R-hydroxy-8Z-octadecasphingenine; N-(2R-hydroxyoctadecanoyl)-1-beta-glucosyl-8E-octadecasphingenine; N-(2R-hydroxy-octadecanoyl)-1-beta-glucosyl-8-hydroxy,9-methylene-sphing-4E-enine; N-(2R-hydroxy-octadecanoyl)-1-beta-glucosyl-8-hydroxy,9-methyl-sphing-4E-enine; N-(2R-hydroxyoctadecanoyl)-1-beta-glucosyl-8Z-octadecasphingenine; N-(2R-hydroxy-octadecanoyl)-1-beta-glucosyl-9-hydroxy-9-methyl-sphing-4E,7E-dienine; N-(2R-hydroxy-octadecanoyl)-1-beta-glucosyl-9-methyl-pentadecasphing-4E,8E-dienine; N-(2R-hydroxy-octadecanoyl)-1-beta-glucosyl-9-methyl-sphing-4E,8E-dienine; N-(2R-hydroxyoctadecanoyl)-4E,8Z-octadecasphingadienine; N-(2R-hydroxypentacosanoyl)-1-beta-glucosyl-4E,8Z-octadecasphingadienine; N-(2R-hydroxypentacosanoyl)-1-beta-glucosyl-4R-hydroxy-8Z-octadecasphingenine; N-(2R-hydroxypentacosanoyl)-4R-hydroxy-sphing-10E-enine; N-(2R-hydroxypentadecanoyl)-1-beta-glucosyl-9-methyl-8E-heneicosphingenine; N-(2R-hydroxy-pentadecanoyl)-1-beta-glucosyl-9-methyl-heneicosasphing-4E,8E-dienine; N-(2R-hydroxy-tetracosanoyl)-1-beta-D-galactosyl-9-methyl-sphing-4E,8E,10E-trienine; N-(2R-hydroxy-tetracosanoyl)-1-beta-D-galactosyl-sphing-4E,8E,10E-trienine; N-(2R-hydroxy-tetracosanoyl)-1-beta-D-glucosyl-9-methyl-sphing-4E,8E,10E-trienine; N-(2R-hydroxy-tetracosanoyl)-1-beta-D-glucosyl-sphing-4E,8E,10E-trienine; N-(2R-hydroxytetracosanoyl)-1-beta-glucosyl-4E,8E-octadecasphingadienine; N-(2R-hydroxytetracosanoyl)-1-beta-glucosyl-4E,8Z-octadecasphingadienine; N-(2R-hydroxytetracosanoyl)-1-beta-glucosyl-4R-hydroxy-8E-octadecasphingenine; N-(2R-hydroxytetracosanoyl)-1-beta-glucosyl-4R-hydroxy-8Z-octadecasphingenine; N-(2R-hydroxytetracosanoyl)-1-beta-glucosyl-8E-octadecasphingenine; N-(2R-hydroxytetracosanoyl)-1-beta-glucosyl-8Z-octadecasphingenine; N-(2R-hydroxy-tetracosanoyl)-1-beta-glucosyl-9-methyl-sphing-4E,8E-dienine; N-(2R-hydroxytetracosanoyl)-4R-hydroxyl-octadecasphinganine; N-(2R-hydroxytetradecanoyl)-4R-hydroxy-octacosasphinganine; N-(2R-hydroxy-tetradecanoyl)-sphing-4E,8E-dienine; N-(2R-hydroxy-tricosanoyl)-1-beta-D-galactosyl-9-methyl-sphing-4E,8E,10E-trienine; N-(2R-hydroxy-tricosanoyl)-1-beta-D-galactosyl-sphing-4E,8E,10E-trienine; N-(2R-hydroxy-tricosanoyl)-1-beta-D-glucosyl-9-methyl-sphing-4E,8E,10E-trienine; N-(2R-hydroxy-tricosanoyl)-1-beta-D-glucosyl-sphing-4E,8E,10E-trienine; N-(2R-hydroxytricosanoyl)-1-beta-glucopyranosyl-4R-hydroxy-14-methyl-pentadecasphinganine; N-(2R-hydroxytricosanoyl)-1-beta-glucopyranosyl-4R-hydroxy-15-methyl-hexadecasphinganine; N-(2R-hydroxytricosanoyl)-1-beta-glucopyranosyl-4R-hydroxy-16-methyl-heptadecasphinganine; N-(2R-hydroxy-tricosanoyl)-1-beta-glucosyl-9-methyl-sphing-4E,8E-dienine; N-(2R-hydroxy-tricosanoyl)-1-beta-glucosyl-eicosasphinganine; N-(2R-hydroxytricosanoyl)-4R-hydroxy-14-methyl-pentadecasphinganine; N-(2R-hydroxytricosanoyl)-4R-hydroxy-15-methyl-hexadecasphinganine; N-(2R-hydroxytricosanoyl)-4R-hydroxy-16-methyl-heptadecasphinganine; N-(2R-hydroytetracosanoyl)-4R-hydroxy-12E-sphingenine; N-(2S-hydroxy-12E-tetracosenoyl)-4R-hydroxysphinganine; N-(2S-hydroxy-3E-octadecenoyl)-1-beta-galactosyl-4E,8E-9-methyl-octadecasphingadienine; N-(2S-hydroxy-3E-octadecenoyl)-1-beta-glucosyl-4E,8E-9-methyl-octadecasphingadienine; N-(2S-hydroxy-octadecanoyl)-1-beta-glucosyl-4E,8E-9-methyl-octadecasphingadienine; N-(2S-hydroxy-tetracosanoyl)-1-alpha-galactosyl-16-methyl-heptdecasphinganine; N-(2S-hydroxy-tetracosanoyl)-1-alpha-galactosyl-16-methyl-octadecasphinganine; N-(2S-hydroxy-tetracosanoyl)-1-alpha-galactosyl-heptadecasphinganine; N-(2S-hydroxy-tetracosanoyl)-1-alpha-galactosyl-hexadecasphinganine; N-(2S-hydroxytetracosanoyl)-4R-hydroxysphinganine-1-phospho-(1′-myo-inositol); N-(30-(9Z,12Z-octadecadienoyloxy)-triacontanoyl)-4E,14Z-sphingadienine; N-(30-(9Z,12Z-octadecadienoyloxy)-triacontanoyl)-4R-hydroxy-docosasphinganine; N-(30-(9Z,12Z-octadecadienoyloxy)-triacontanoyl)-4R-hydroxy-eicosasphinganine; N-(30-(9Z,12Z-octadecadienoyloxy)-triacontanoyl)-4R-hydroxy-eicosasphinganine; N-(30-(9Z,12Z-octadecadienoyloxy)-triacontanoyl)-4R-hydroxy-heptadecasphinganine; N-(30-(9Z,12Z-octadecadienoyloxy)-triacontanoyl)-4R-hydroxy-hexadecasphinganine; N-(30-(9Z,12Z-octadecadienoyloxy)-triacontanoyl)-4R-hydroxy-nonadecasphinganine; N-(30-(9Z,12Z-octadecadienoyloxy)-triacontanoyl)-4R-hydroxysphinganine; N-(30-(9Z,12Z-octadecadienoyloxy)-triacontanoyl)-6R-hydroxy-docosasphing-4E-enine; N-(30-(9Z,12Z-octadecadienoyloxy)-triacontanoyl)-6R-hydroxy-eicosasphing-4E-enine; N-(30-(9Z,12Z-octadecadienoyloxy)-triacontanoyl)-6R-hydroxy-heneicosasphing-4E-enine; N-(30-(9Z,12Z-octadecadienoyloxy)-triacontanoyl)-6R-hydroxy-heptadecasphing-4E-enine; N-(30-(9Z,12Z-octadecadienoyloxy)-triacontanoyl)-6R-hydroxy-nonadecasphing-4E-enine; N-(30-(9Z,12Z-octadecadienoyloxy)-triacontanoyl)-6R-hydroxy-sphing-4E-enine; N-(30-(9Z,12Z-octadecadienoyloxy)-triacontanoyl)-docosasphing-4E-enine; N-(30-(9Z,12Z-octadecadienoyloxy)-triacontanoyl)-docosasphinganine; N-(30-(9Z,12Z-octadecadienoyloxy)-triacontanoyl)-eicosasphing-4E-enine; N-(30-(9Z,12Z-octadecadienoyloxy)-triacontanoyl)-eicosasphinganine; N-(30-(9Z,12Z-octadecadienoyloxy)-triacontanoyl)-heneicosasphing-4E-enine; N-(30-(9Z,12Z-octadecadienoyloxy)-triacontanoyl)-heneicosasphinganine; N-(30-(9Z,12Z-octadecadienoyloxy)-triacontanoyl)-heptadecasphing-4E-enine; N-(30-(9Z,12Z-octadecadienoyloxy)-triacontanoyl)-hexadecasphing-4E-enine; N-(30-(9Z,12Z-octadecadienoyloxy)-triacontanoyl)-nonadecasphing-4E-enine; N-(30-(9Z,12Z-octadecadienoyloxy)-triacontanoyl)-nonadecasphinganine; N-(30-(9Z,12Z-octadecadienoyloxy)-triacontanoyl)-sphing-4-enine; N-(30-(9Z,12Z-octadecadienoyloxy)-triacontanoyl)-sphinganine; N-(30-(9Z,12Z-octadecadienoyloxy)-triacontanoyl)-triacosasphinganine; N-(30-hydroxy-triacontanoyl)-4E,14Z-sphingadienine; N-(30-hydroxy-triacontanoyl)-4R-hydroxysphinganine; N-(30-hydroxy-triacontanoyl)-6R-hydroxy-sphing-4E-enine; N-(30-hydroxy-triacontanoyl)-sphing-4-enine; N-(30-hydroxy-triacontanoyl)-sphinganine; N-(31-(9Z,12Z-octadecadienoyloxy)-hentriacontanoyl)-4E,14Z-sphingadienine; N-(31-(9Z,12Z-octadecadienoyloxy)-hentriacontanoyl)-4R-hydroxy-docosasphinganine; N-(31-(9Z,12Z-octadecadienoyloxy)-hentriacontanoyl)-4R-hydroxy-eicosasphinganine; N-(31-(9Z,12Z-octadecadienoyloxy)-hentriacontanoyl)-4R-hydroxy-eicosasphinganine; N-(31-(9Z,12Z-octadecadienoyloxy)-hentriacontanoyl)-4R-hydroxy-heptadecasphinganine; N-(31-(9Z,12Z-octadecadienoyloxy)-hentriacontanoyl)-4R-hydroxysphinganine; N-(31-(9Z,12Z-octadecadienoyloxy)-hentriacontanoyl)-6R-hydroxy-eicosasphing-4E-enine; N-(31-(9Z,12Z-octadecadienoyloxy)-hentriacontanoyl)-6R-hydroxy-heneicosasphing-4E-enine; N-(31-(9Z,12Z-octadecadienoyloxy)-hentriacontanoyl)-6R-hydroxy-heptadecasphing-4E-enine; N-(31-(9Z,12Z-octadecadienoyloxy)-hentriacontanoyl)-6R-hydroxy-nonadecasphing-4E-enine; N-(31-(9Z,12Z-octadecadienoyloxy)-hentriacontanoyl)-6R-hydroxy-sphing-4E-enine; N-(31-(9Z,12Z-octadecadienoyloxy)-hentriacontanoyl)-docosasphing-4E-enine; N-(31-(9Z,12Z-octadecadienoyloxy)-hentriacontanoyl)-docosasphinganine; N-(31-(9Z,12Z-octadecadienoyloxy)-hentriacontanoyl)-eicosasphing-4E-enine; N-(31-(9Z,12Z-octadecadienoyloxy)-hentriacontanoyl)-eicosasphinganine; N-(31-(9Z,12Z-octadecadienoyloxy)-hentriacontanoyl)-heneicosasphing-4E-enine; N-(31-(9Z,12Z-octadecadienoyloxy)-hentriacontanoyl)-heneicosasphinganine; N-(31-(9Z,12Z-octadecadienoyloxy)-hentriacontanoyl)-heptadecasphing-4E-enine; N-(31-(9Z,12Z-octadecadienoyloxy)-hentriacontanoyl)-hexadecasphing-4E-enine; N-(31-(9Z,12Z-octadecadienoyloxy)-hentriacontanoyl)-nonadecasphing-4E-enine; N-(31-(9Z,12Z-octadecadienoyloxy)-hentriacontanoyl)-nonadecasphinganine; N-(31-(9Z,12Z-octadecadienoyloxy)-hentriacontanoyl)-sphing-4E-enine; N-(31-(9Z,12Z-octadecadienoyloxy)-hentriacontanoyl)-sphinganine; N-(31-(9Z,12Z-octadecadienoyloxy)-hentriacontanoyl)-triacosasphinganine; N-(31-hydroxy-hentriacontanoyl)-4E,14Z-sphingadienine; N-(31-hydroxy-hentriacontanoyl)-4R-hydroxysphinganine; N-(31-hydroxy-hentriacontanoyl)-6R-hydroxy-sphing-4E-enine; N-(31-hydroxy-hentriacontanoyl)-sphing-4-enine; N-(31-hydroxy-hentriacontanoyl)-sphinganine; N-(32-(9Z,12Z-octadecadienoyloxy)-dotriacontanoyl)-1-beta-D-glucosyl-sphing-4E-enine; N-(32-(9Z,12Z-octadecadienoyloxy)-dotriacontanoyl)-4E,14Z-sphingadienine; N-(32-(9Z,12Z-octadecadienoyloxy)-dotriacontanoyl)-4-hydroxysphinganine; N-(32-(9Z,12Z-octadecadienoyloxy)-dotriacontanoyl)-4R-hydroxy-docosasphinganine; N-(32-(9Z,12Z-octadecadienoyloxy)-dotriacontanoyl)-4R-hydroxy-eicosasphinganine; N-(32-(9Z,12Z-octadecadienoyloxy)-dotriacontanoyl)-4R-hydroxy-eicosasphinganine; N-(32-(9Z,12Z-octadecadienoyloxy)-dotriacontanoyl)-4R-hydroxy-heptadecasphinganine; N-(32-(9Z,12Z-octadecadienoyloxy)-dotriacontanoyl)-4R-hydroxy-nonadecasphinganine; N-(32-(9Z,12Z-octadecadienoyloxy)-dotriacontanoyl)-4R-hydroxysphinganine; N-(32-(9Z,12Z-octadecadienoyloxy)-dotriacontanoyl)-6R-hydroxy-eicosasphing-4E-enine; N-(32-(9Z,12Z-octadecadienoyloxy)-dotriacontanoyl)-6R-hydroxy-heptadecasphing-4E-enine; N-(32-(9Z,12Z-octadecadienoyloxy)-dotriacontanoyl)-6R-hydroxy-nonadecasphing-4E-enine; N-(32-(9Z,12Z-octadecadienoyloxy)-dotriacontanoyl)-6R-hydroxy-sphing-4E-enine; N-(32-(9Z,12Z-octadecadienoyloxy)-dotriacontanoyl)-docosasphing-4E-enine; N-(32-(9Z,12Z-octadecadienoyloxy)-dotriacontanoyl)-docosasphinganine; N-(32-(9Z,12Z-octadecadienoyloxy)-dotriacontanoyl)-eicosasphing-4E-enine; N-(32-(9Z,12Z-octadecadienoyloxy)-dotriacontanoyl)-eicosasphinganine; N-(32-(9Z,12Z-octadecadienoyloxy)-dotriacontanoyl)-heneicosasphing-4E-enine; N-(32-(9Z,12Z-octadecadienoyloxy)-dotriacontanoyl)-heneicosasphinganine; N-(32-(9Z,12Z-octadecadienoyloxy)-dotriacontanoyl)-heptadecasphing-4E-enine; N-(32-(9Z,12Z-octadecadienoyloxy)-dotriacontanoyl)-heptadecasphinganine; N-(32-(9Z,12Z-octadecadienoyloxy)-dotriacontanoyl)-hexadecasphing-4E-enine; N-(32-(9Z,12Z-octadecadienoyloxy)-dotriacontanoyl)-nonadecasphing-4E-enine; N-(32-(9Z,12Z-octadecadienoyloxy)-dotriacontanoyl)-nonadecasphinganine; N-(32-(9Z,12Z-octadecadienoyloxy)-dotriacontanoyl)-sphing-4E-enine; N-(32-(9Z,12Z-octadecadienoyloxy)-dotriacontanoyl)-sphinganine; N-(32-hydroxy-dotriacontanoyl)-4E,14Z-sphingadienine; N-(32-hydroxy-dotriacontanoyl)-4R-hydroxysphinganine; N-(32-hydroxy-dotriacontanoyl)-6R-hydroxy-sphing-4E-enine; N-(32-hydroxy-dotriacontanoyl)-sphing-4-enine; N-(32-hydroxy-dotriacontanoyl)-sphinganine; N-(33-(9Z,12Z-octadecadienoyloxy)-tritriacontanoyl)-4R-hydroxysphinganine; N-(33-(9Z,12Z-octadecadienoyloxy)-tritriacontanoyl)-6R-hydroxy-sphing-4E-enine; N-(33-(9Z,12Z-octadecadienoyloxy)-tritriacontanoyl)-eicosasphing-4E-enine; N-(33-(9Z,12Z-octadecadienoyloxy)-tritriacontanoyl)-heneicosasphing-4E-enine; N-(33-(9Z,12Z-octadecadienoyloxy)-tritriacontanoyl)-heneicosasphinganine; N-(33-(9Z,12Z-octadecadienoyloxy)-tritriacontanoyl)-heptadecasphing-4E-enine; N-(33-(9Z,12Z-octadecadienoyloxy)-tritriacontanoyl)-heptadecasphinganine; N-(33-(9Z,12Z-octadecadienoyloxy)-tritriacontanoyl)-hexadecasphinganine; N-(33-(9Z,12Z-octadecadienoyloxy)-tritriacontanoyl)-nonadecasphing-4E-enine; N-(33-(9Z,12Z-octadecadienoyloxy)-tritriacontanoyl)-nonadecasphinganine; N-(33-(9Z,12Z-octadecadienoyloxy)-tritriacontanoyl)-sphing-4E-enine; N-(33-(9Z,12Z-octadecadienoyloxy)-tritriacontanoyl)-sphinganine; N-(33-hydroxy-tritriacontanoyl)-4R-hydroxysphinganine; N-(33-hydroxy-tritriacontanoyl)-6R-hydroxy-sphing-4E-enine; N-(33-hydroxy-tritriacontanoyl)-sphing-4-enine; N-(33-hydroxy-tritriacontanoyl)-sphinganine; N-(34-(9Z,12Z-octadecadienoyloxy)-tetratriacontanoyl)-4E,14Z-sphingadienine; N-(34-(9Z,12Z-octadecadienoyloxy)-tetratriacontanoyl)-4R-hydroxysphinganine; N-(34-(9Z,12Z-octadecadienoyloxy)-tetratriacontanoyl)-6R-hydroxy-sphing-4E-enine; N-(34-(9Z,12Z-octadecadienoyloxy)-tetratriacontanoyl)-eicosasphing-4E-enine; N-(34-(9Z,12Z-octadecadienoyloxy)-tetratriacontanoyl)-heptadecasphing-4E-enine; N-(34-(9Z,12Z-octadecadienoyloxy)-tetratriacontanoyl)-heptadecasphinganine; N-(34-(9Z,12Z-octadecadienoyloxy)-tetratriacontanoyl)-hexadecasphinganine; N-(34-(9Z,12Z-octadecadienoyloxy)-tetratriacontanoyl)-nonadecasphing-4E-enine; N-(34-(9Z,12Z-octadecadienoyloxy)-tetratriacontanoyl)-nonadecasphinganine; N-(34-(9Z,12Z-octadecadienoyloxy)-tetratriacontanoyl)-sphing-4E-enine; N-(34-(9Z,12Z-octadecadienoyloxy)-tetratriacontanoyl)-sphinganine; N-(34-hydroxy-tetratriacontanoyl)-4E,14Z-sphingadienine; N-(34-hydroxy-tetratriacontanoyl)-4R-hydroxysphinganine; N-(34-hydroxy-tetratriacontanoyl)-6R-hydroxy-sphing-4E-enine; N-(34-hydroxy-tetratriacontanoyl)-sphing-4-enine; N-(34-hydroxy-tetratriacontanoyl)-sphinganine; N-(34S-methyl-5Z,9Z,12Z,15Z,18Z,21Z-hexatriacontahexaenoyl)-1-sulfo-beta-D-fucosyl-(16R-methyl-sphing-4-enine); N-(35-(9Z,12Z-octadecadienoyloxy)-pentatriacontanoyl)-sphing-4E-enine; N-(35-hydroxy-pentatriacontanoyl)-6R-hydroxy-sphing-4E-enine; N-(35-hydroxy-pentatriacontanoyl)-sphing-4-enine; N-(36-(9Z,12Z-octadecadienoyloxy)-hexatriacontanoyl)-6R-hydroxy-sphing-4E-enine; N-(36-(9Z,12Z-octadecadienoyloxy)-hexatriacontanoyl)-sphing-4E-enine; N-(36-(9Z,12Z-octadecadienoyloxy)-hexatriacontanoyl)-sphinganine; N-(36-hydroxy-hexatriacontanoyl)-4R-hydroxysphinganine; N-(36-hydroxy-hexatriacontanoyl)-6R-hydroxy-sphing-4E-enine; N-(36-hydroxy-hexatriacontanoyl)-sphing-4-enine; N-(36-hydroxy-hexatriacontanoyl)-sphinganine; N-(3E-hexadecenoyl)-deoxysphing-4-enine-1-sulfonate; N-(3-hydroxy-13-methyltetradecanoyl)-15-methylhexadecasphinganine-1-phospho-(1′-myo-inositol); N-(3-hydroxy-13-methyltetradecanoyl)-15-methylhexadecasphinganine-1-phosphoethanolamine; N-(3-hydroxy-15-methylhexadecanoyl)-17-methylsphinganine-1-phosphoethanolamine; N-(3R-hydroxy-docosanoyl)-sphing-4-enine; N-(3R-hydroxy-eicosanoyl)-sphing-4-enine; N-(3R-hydroxy-heneicosanoyl)-sphing-4-enine; N-(3R-hydroxy-hentriacontanoyl)-sphing-4-enine; N-(3R-hydroxy-heptacosanoyl)-sphing-4-enine; N-(3R-hydroxy-heptadecanoyl)-sphing-4-enine; N-(3R-hydroxy-hexacosanoyl)-sphing-4-enine; N-(3R-hydroxy-hexadecanoyl)-sphing-4-enine; N-(3R-hydroxy-nonacosanoyl)-sphing-4-enine; N-(3R-hydroxy-nonadecanoyl)-sphing-4-enine; N-(3R-hydroxy-octacosanoyl)-sphing-4-enine; N-(3R-hydroxy-octadecanoyl)-sphing-4-enine; N-(3R-hydroxy-pentacosanoyl)-sphing-4-enine; N-(3R-hydroxy-tetracosanoyl)-sphing-4-enine; N-(3R-hydroxy-triacontanoyl)-sphing-4-enine; N-(3R-hydroxy-tricosanoyl)-sphing-4-enine; N-(9Z,12Z-octadecadienoyl)-sphinganine-1-phosphocholine; N-(9Z-heptadecenoyl)-nonadecasphing-4-enine-1-phosphocholine; N-(9Z-hexadecenoyl)-sphing-4-enine-1-phosphocholine; N-(9Z-octadecenoyl)-1-beta-glucosyl-4E,6E-tetradecasphingadienine; N-(9Z-octadecenoyl)-1-beta-glucosyl-sphing-4-enine; N-(9Z-octadecenoyl)-1-beta-glucosyl-sphinganine; N-(9Z-octadecenoyl)-1-beta-glucosyl-tetradecasphing-4-enine; N-(9Z-octadecenoyl)-1-beta-lactosyl-sphing-4-enine; N-(9Z-octadecenoyl)-1-beta-lactosyl-sphinganine; N-(9Z-octadecenoyl)-4E,14Z-sphingadienine; N-(9Z-octadecenoyl)-4E,14Z-sphingadienine-1-phosphocholine; N-(9Z-octadecenoyl)-4E,6E-hexadecasphingadienine-1-phosphoethanolamine; N-(9Z-octadecenoyl)-4E,6E-tetradecasphingadienine; N-(9Z-octadecenoyl)-4E,6E-tetradecasphingadienine-1-phosphoethanolamine; N-(9Z-octadecenoyl)-hexadecasphing-4-enine-1-phosphocholine; N-(9Z-octadecenoyl)-sphing-4-enine; N-(9Z-octadecenoyl)-sphing-4-enine-1-phosphocholine; N-(9Z-octadecenoyl)-sphinganine; N-(9Z-octadecenoyl)-sphinganine-1-phosphocholine; N-(9Z-octadecenoyl)-tetradecasphing-4-enine; N-(9Z-octadecenoyl)-tetradecasphing-4-enine-1-phosphoethanolamine; N-(acetyl)-sphing-4-enine; N-(acetyl)-sphing-4-enine-1-phosphate; N-(acyl)-sphing-4-enine-1-(2-aminoethylphosphonate); N-(acyl)-sphing-4-enine-1-phosphate; N-(acyl)-sphing-4-enine-1-phosphoethanolamine; N-(docosanoyl)-1-beta-(2′,3′,4′,6′-tetra-O-acetyl-galactosyl)-3-O-acetyl-sphing-4-enine; N-(docosanoyl)-1-beta-galactosyl-4E,14Z-sphingadienine; N-(docosanoyl)-1-beta-galactosyl-hexadecasphing-4-enine; N-(docosanoyl)-1-beta-galactosyl-sphinganine; N-(docosanoyl)-1beta-glucosyl-4E,14Z-sphingadienine; N-(docosanoyl)-1-beta-glucosyl-4E,6E-hexadecasphingadienine; N-(docosanoyl)-1-beta-glucosyl-4E,6E-pentadecasphingadienine; N-(docosanoyl)-1-beta-glucosyl-4E,6E-tetradecasphingadienine; N-(docosanoyl)-1-beta-glucosyl-eicosasphinganine; N-(docosanoyl)-1-beta-glucosyl-hexadecasphing-4-enine; N-(docosanoyl)-1-beta-glucosyl-pentadecasphing-4-enine; N-(docosanoyl)-1-beta-glucosyl-sphing-4-enine; N-(docosanoyl)-1-beta-glucosyl-sphinganine; N-(docosanoyl)-1-beta-glucosyl-tetradecasphing-4-enine; N-(docosanoyl)-1-beta-lactosyl-hexadecasphinganine; N-(docosanoyl)-1-beta-lactosyl-sphing-4-enine; N-(docosanoyl)-1-beta-lactosyl-sphinganine; N-(docosanoyl)-1-deoxysphing-4-enine; N-(docosanoyl)-1-deoxysphinganine; N-(docosanoyl)-4E,12Z-sphingadienine-1-phosphocholine; N-(docosanoyl)-4E,14Z-sphingadienine; N-(docosanoyl)-4E,6E-hexadecasphingadienine; N-(docosanoyl)-4E,6E-hexadecasphingadienine-1-phosphoethanolamine; N-(docosanoyl)-4E,6E-pentadecasphingadienine; N-(docosanoyl)-4E,6E-pentadecasphingadienine-1-phosphoethanolamine; N-(docosanoyl)-4E,6E-tetradecasphingadienine; N-(docosanoyl)-4E,6E-tetradecasphingadienine-1-phosphoethanolamine; N-(docosanoyl)-4R-hydroxy-eicosasphinganine; N-(docosanoyl)-4R-hydroxyeicosasphinganine-1-O-[D-mannopyranosyl-alpha1-2-myo-inositol-1-phosphate]; N-(docosanoyl)-4R-hydroxyeicosasphinganine-1-O-[myo-inositol-1-phosphoryl-6-D-mannopyranosyl-alpha1-2-myo-inositol-1-phosphate]; N-(docosanoyl)-4R-hydroxyeicosasphinganine-1-phospho-(1′-myo-inositol); N-(docosanoyl)-4R-hydroxysphinganine; N-(docosanoyl)-4R-hydroxysphinganine-1-O-[D-mannopyranosyl-alpha1-2-myo-inositol-1-phosphate]; N-(docosanoyl)-4R-hydroxysphinganine-1-O-[myo-inositol-1-phosphoryl-6-D-mannopyranosyl-alpha1-2-myo-inositol-1-phosphate]; N-(docosanoyl)-4R-hydroxysphinganine-1-phospho-(1′-myo-inositol); N-(docosanoyl)-6R-hydroxy-sphing-4E-enine; N-(docosanoyl)-eicosasphinganine; N-(docosanoyl)-eicosasphinganine-1-O-[D-mannopyranosyl-alpha1-2-myo-inositol-1-phosphate]; N-(docosanoyl)-eicosasphinganine-1-O-[myo-inositol-1-phosphoryl-6-D-mannopyranosyl-alpha1-2-myo-inositol-1-phosphate]; N-(docosanoyl)-eicosasphinganine-1-phospho-(1′-myo-inositol); N-(docosanoyl)-heptadecasphing-4-enine-1-phosphocholine; N-(docosanoyl)-heptadecasphinganine-1-phosphocholine; N-(docosanoyl)-hexadecasphing-4-enine; N-(docosanoyl)-hexadecasphing-4-enine-1-phosphocholine; N-(docosanoyl)-hexadecasphing-4-enine-1-phosphoethanolamine; N-(docosanoyl)-hexadecasphinganine; N-(docosanoyl)-hexadecasphinganine-1-phosphocholine; N-(docosanoyl)-pentadecasphing-4-enine; N-(docosanoyl)-pentadecasphing-4-enine-1-phosphoethanolamine; N-(docosanoyl)-sphing-4-enine; N-(docosanoyl)-sphing-4-enine-1-phosphate; N-(docosanoyl)-sphing-4-enine-1-phospho-(1′-myo-inositol); N-(docosanoyl)-sphing-4-enine-1-phosphocholine; N-(docosanoyl)-sphinganine; N-(docosanoyl)-sphinganine-1-O-[D-mannopyranosyl-alpha1-2-myo-inositol-1-phosphate]; N-(docosanoyl)-sphinganine-1-O-[myo-inositol-1-phosphoryl-6-D-mannopyranosyl-alpha1-2-myo-inositol-1-phosphate]; N-(docosanoyl)-sphinganine-1-phospho-(1′-myo-inositol); N-(docosanoyl)-sphinganine-1-phosphocholine; N-(docosanoyl)-tetradecasphing-4-enine; N-(docosanoyl)-tetradecasphing-4-enine-1-phosphoethanolamine; N-(dodecanoyl)-1-beta-glucosyl-sphing-4-enine; N-(dodecanoyl)-1-beta-lactosyl-sphing-4-enine; N-(dodecanoyl)-sphing-4-enine; N-(dodecanoyl)-sphing-4-enine-1-phosphate; N-(dodecanoyl)-sphing-4-enine-1-phosphocholine; N-(dodecanoyl)-sphinganine-1-phosphocholine; N-(dotriacontanoyl)-4E,14Z-sphingadienine; N-(dotriacontanoyl)-4R-hydroxysphinganine; N-(dotriacontanoyl)-6R-hydroxy-sphing-4E-enine; N-(dotriacontanoyl)-sphing-4-enine; N-(dotriacontanoyl)-sphinganine; N-(eicosanoyl)-1-beta-(2′,3′,4′,6′-tetra-O-acetyl-galactosyl)-3-O-acetyl-sphing-4-enine; N-(eicosanoyl)-1-beta-galactosyl-4E,14Z-sphingadienine; N-(eicosanoyl)-1-beta-galactosyl-sphinganine; N-(eicosanoyl)-1beta-glucosyl-4E,14Z-sphingadienine; N-(eicosanoyl)-1-beta-glucosyl-4E,6E-hexadecasphingadienine; N-(eicosanoyl)-1-beta-glucosyl-4E,6E-pentadecasphingadienine; N-(eicosanoyl)-1-beta-glucosyl-4E,6E-tetradecasphingadienine; N-(eicosanoyl)-1-beta-glucosyl-hexadecasphing-4-enine; N-(eicosanoyl)-1-beta-glucosyl-pentadecasphing-4-enine; N-(eicosanoyl)-1-beta-glucosyl-sphing-4-enine; N-(eicosanoyl)-1-beta-glucosyl-sphinganine; N-(eicosanoyl)-1-beta-glucosyl-tetradecasphing-4-enine; N-(eicosanoyl)-1-beta-lactosyl-hexadecasphinganine; N-(eicosanoyl)-1-beta-lactosyl-sphing-4-enine; N-(eicosanoyl)-1-beta-lactosyl-sphinganine; N-(eicosanoyl)-1-deoxysphing-4-enine; N-(eicosanoyl)-1-deoxysphinganine; N-(eicosanoyl)-4E,14Z-sphingadienine; N-(eicosanoyl)-4E,14Z-sphingadienine-1-phosphocholine; N-(eicosanoyl)-4E,6E-hexadecasphingadienine; N-(eicosanoyl)-4E,6E-hexadecasphingadienine-1-phosphoethanolamine; N-(eicosanoyl)-4E,6E-pentadecasphingadienine; N-(eicosanoyl)-4E,6E-pentadecasphingadienine-1-phosphoethanolamine; N-(eicosanoyl)-4E,6E-tetradecasphingadienine; N-(eicosanoyl)-4E,6E-tetradecasphingadienine-1-phosphoethanolamine; N-(eicosanoyl)-4R-hydroxy-eicosasphinganine; N-(eicosanoyl)-4R-hydroxyeicosasphinganine-1-O-[D-mannopyranosyl-alpha1-2-myo-inositol-1-phosphate]; N-(eicosanoyl)-4R-hydroxyeicosasphinganine-1-O-[myo-inositol-1-phosphoryl-6-D-mannopyranosyl-alpha1-2-myo-inositol-1-phosphate]; N-(eicosanoyl)-4R-hydroxyeicosasphinganine-1-phospho-(1′-myo-inositol); N-(eicosanoyl)-4R-hydroxysphinganine; N-(eicosanoyl)-4R-hydroxysphinganine-1-O-[D-mannopyranosyl-alpha1-2-myo-inositol-1-phosphate]; N-(eicosanoyl)-4R-hydroxysphinganine-1—O-[myo-inositol-1-phosphoryl-6-D-mannopyranosyl-alpha1-2-myo-inositol-1-phosphate]; N-(eicosanoyl)-4R-hydroxysphinganine-1-phospho-(1′-myo-inositol); N-(eicosanoyl)-6R-hydroxy-sphing-4E-enine; N-(eicosanoyl)-eicosasphinganine; N-(eicosanoyl)-eicosasphinganine-1-O-[D-mannopyranosyl-alpha1-2-myo-inositol-1-phosphate]; N-(eicosanoyl)-eicosasphinganine-1-O-[myo-inositol-1-phosphoryl-6-D-mannopyranosyl-alpha1-2-myo-inositol-1-phosphate]; N-(eicosanoyl)-eicosasphinganine-1-phospho-(1′-myo-inositol); N-(eicosanoyl)-hexadecasphing-4-enine; N-(eicosanoyl)-hexadecasphing-4-enine-1-phosphocholine; N-(eicosanoyl)-hexadecasphing-4-enine-1-phosphoethanolamine; N-(eicosanoyl)-hexadecasphinganine-1-phosphocholine; N-(eicosanoyl)-nonadecasphing-4-enine-1-phosphocholine; N-(eicosanoyl)-pentadecasphing-4-enine; N-(eicosanoyl)-pentadecasphing-4-enine-1-phosphoethanolamine; N-(eicosanoyl)-sphing-4-enine; N-(eicosanoyl)-sphing-4-enine-1-phosphate; N-(eicosanoyl)-sphing-4-enine-1-phosphocholine; N-(eicosanoyl)-sphinganine; N-(eicosanoyl)-sphinganine-1-O-[D-mannopyranosyl-alpha1-2-myo-inositol-1-phosphate]; N-(eicosanoyl)-sphinganine-1-O-[myo-inositol-1-phosphoryl-6-D-mannopyranosyl-alpha1-2-myo-inositol-1-phosphate]; N-(eicosanoyl)-sphinganine-1-phospho-(1′-myo-inositol); N-(eicosanoyl)-sphinganine-1-phosphocholine; N-(eicosanoyl)-tetradecasphing-4-enine; N-(eicosanoyl)-tetradecasphing-4-enine-1-phosphoethanolamine; N-(heneicosanoyl)-1-beta-galactosyl-4E,14Z-sphingadienine; N-(heneicosanoyl)-1beta-glucosyl-4E,14Z-sphingadienine; N-(heneicosanoyl)-4E,14Z-sphingadienine; N-(heneicosanoyl)-4E,14Z-sphingadienine-1-phosphocholine; N-(heneicosanoyl)-4E,6E-hexadecasphingadienine-1-phosphoethanolamine; N-(heneicosanoyl)-4E,6E-tetradecasphingadienine-1-phosphoethanolamine; N-(heneicosanoyl)-4R-hydroxysphinganine; N-(heneicosanoyl)-6R-hydroxy-sphing-4E-enine; N-(heneicosanoyl)-hexadecasphing-4-enine-1-phosphoethanolamine; N-(heneicosanoyl)-sphing-4-enine; N-(heneicosanoyl)-sphing-4-enine-1-phosphocholine; N-(heneicosanoyl)-sphinganine; N-(heneicosanoyl)-tetradecasphing-4-enine-1-phosphoethanolamine; N-(hentriacontanoyl)-4R-hydroxysphinganine; N-(hentriacontanoyl)-6R-hydroxy-sphing-4E-enine; N-(hentriacontanoyl)-sphing-4-enine; N-(hentriacontanoyl)-sphinganine; N-(heptacosanoyl)-4E,14Z-sphingadienine; N-(heptacosanoyl)-4R-hydroxysphinganine; N-(heptacosanoyl)-6R-hydroxy-sphing-4E-enine; N-(heptacosanoyl)-heptadecasphinganine-1-phosphocholine; N-(heptacosanoyl)-sphing-4-enine; N-(heptacosanoyl)-sphinganine; N-(heptadecanoyl)-4E,14Z-sphingadienine-1-phosphocholine; N-(heptadecanoyl)-4R-hydroxysphinganine; N-(heptadecanoyl)-6R-hydroxy-sphing-4E-enine; N-(heptadecanoyl)-heptadecasphing-4-enine-1-phosphocholine; N-(heptadecanoyl)-hexadecasphing-4-enine-1-phosphocholine; N-(heptadecanoyl)-hexdecasphing-4-enine; N-(heptadecanoyl)-nonadecasphinganine-1-phosphocholine; N-(heptadecanoyl)-sphing-4-enine; N-(heptadecanoyl)-sphing-4-enine-1-phosphocholine; N-(heptadecanoyl)-sphinganine; N-(heptadecanoyl)-sphinganine-1-phosphocholine; N-(hexacosanoyl)-1-beta-galactosyl-sphinganine; N-(hexacosanoyl)-1-beta-glucosyl-sphing-4-enine; N-(hexacosanoyl)-1-beta-glucosyl-sphinganine; N-(hexacosanoyl)-1-beta-lactosyl-sphinganine; N-(hexacosanoyl)-1-b-lactosyl-sphing-4-enine; N-(hexacosanoyl)-1-deoxysphing-4-enine; N-(hexacosanoyl)-1-deoxysphinganine; N-(hexacosanoyl)-4E,14Z-sphingadienine; N-(hexacosanoyl)-4E,6E-tetradecasphingadienine-1-phosphoethanolamine; N-(hexacosanoyl)-4R-hydroxy-eicosasphinganine; N-(hexacosanoyl)-4R-hydroxyeicosasphinganine-1-O-[D-mannopyranosyl-alpha1-2-myo-inositol-1-phosphate]; N-(hexacosanoyl)-4R-hydroxyeicosasphinganine-1-O-[myo-inositol-1-phosphoryl-6-D-mannopyranosyl-alpha1-2-myo-inositol-1-phosphate]; N-(hexacosanoyl)-4R-hydroxyeicosasphinganine-1-phospho-(1′-myo-inositol); N-(hexacosanoyl)-4R-hydroxysphinganine; N-(hexacosanoyl)-4R-hydroxysphinganine-1-O-[D-mannopyranosyl-alpha1-2-myo-inositol-1-phosphate]; N-(hexacosanoyl)-4R-hydroxysphinganine-1-phospho-(1′-[2-amino-2-deoxy-D-glucopyranosyl-alpha1-6-D-myo-inositol]); N-(hexacosanoyl)-4R-hydroxysphinganine-1-phospho-(1′-myo-inositol); N-(hexacosanoyl)-6R-hydroxy-sphing-4E-enine; N-(hexacosanoyl)-eicosasphinganine; N-(hexacosanoyl)-eicosasphinganine-1-O-[D-mannopyranosyl-alpha1-2-myo-inositol-1-phosphate]; N-(hexacosanoyl)-eicosasphinganine-1-O-[myo-inositol-1-phosphoryl-6-D-mannopyranosyl-alpha1-2-myo-inositol-1-phosphate]; N-(hexacosanoyl)-eicosasphinganine-1-phospho-(1′-myo-inositol); N-(hexacosanoyl)-sphing-4-enine; N-(hexacosanoyl)-sphing-4-enine-1-phosphate; N-(hexacosanoyl)-sphing-4-enine-1-phosphocholine; N-(hexacosanoyl)-sphinganine; N-(hexacosanoyl)-sphinganine-1-O-[D-mannopyranosyl-alpha1-2-myo-inositol-1-phosphate]; N-(hexacosanoyl)-sphinganine-1-O-[myo-inositol-1-phosphoryl-6-D-mannopyranosyl-alpha1-2-myo-inositol-1-phosphate]; N-(hexacosanoyl)-sphinganine-1-phospho-(1′-myo-inositol); N-(hexacosanoyl)-sphinganine-1-phosphocholine; N-(hexacosanoyl)-tetradecasphing-4-enine; N-(hexacosanoyl)-tetradecasphing-4-enine-1-phosphoethanolamine; N-(hexadecanoyl)-1-alpha-glucosyl-9-methyl-4E,8E,10E-sphingatrienine; N-(hexadecanoyl)-1-alpha-glucosyl-9-methyl-4E,8E-heptadecasphingadienine; N-(hexadecanoyl)-1-alpha-glucosyl-9-methyl-4E,8E-sphingadienine; N-(hexadecanoyl)-1-beta-galactosyl-4E,14Z-sphingadienine; N-(hexadecanoyl)-1-beta-galactosyl-sphinganine; N-(hexadecanoyl)-1beta-glucosyl-4E,14Z-sphingadienine; N-(hexadecanoyl)-1-beta-glucosyl-4E,6E-tetradecasphingadienine; N-(hexadecanoyl)-1-beta-glucosyl-4E,8Z-sphingadienine; N-(hexadecanoyl)-1-beta-glucosyl-sphing-4-enine; N-(hexadecanoyl)-1-beta-glucosyl-sphinganine; N-(hexadecanoyl)-1-beta-glucosyl-tetradecasphing-4-enine; N-(hexadecanoyl)-1-beta-lactosyl-docosasphinganine; N-(hexadecanoyl)-1-beta-lactosyl-hexadecasphinganine; N-(hexadecanoyl)-1-beta-lactosyl-sphing-4-enine; N-(hexadecanoyl)-1-beta-lactosyl-sphinganine; N-(hexadecanoyl)-1-beta-lactosyl-tetradecasphing-4-enine; N-(hexadecanoyl)-1-deoxysphing-4-enine; N-(hexadecanoyl)-1-deoxysphinganine; N-(hexadecanoyl)-4E,14Z-sphingadienine; N-(hexadecanoyl)-4E,14Z-sphingadienine-1-phosphocholine; N-(hexadecanoyl)-4E,6E-hexadecasphingadienine-1-phosphoethanolamine; N-(hexadecanoyl)-4E,6E-tetradecasphingadienine; N-(hexadecanoyl)-4E,6E-tetradecasphingadienine-1-phosphoethanolamine; N-(hexadecanoyl)-4E-pentadeca-sphingenine; N-(hexadecanoyl)-4R-hydroxypentadecasphinganine; N-(hexadecanoyl)-4R-hydroxy-eicosasphinganine; N-(hexadecanoyl)-4R-hydroxyeicosasphinganine-1-O-[D-mannopyranosyl-alpha1-2-myo-inositol-1-phosphate]; N-(hexadecanoyl)-4R-hydroxyeicosasphinganine-1-O-[myo-inositol-1-phosphoryl-6-D-mannopyranosyl-alpha1-2-myo-inositol-1-phosphate]; N-(hexadecanoyl)-4R-hydroxyeicosasphinganine-1-phospho-(1′-myo-inositol); N-(hexadecanoyl)-4R-hydroxysphinganine; N-(hexadecanoyl)-4R-hydroxysphinganine-1-O-[D-mannopyranosyl-alpha1-2-myo-inositol-1-phosphate]; N-(hexadecanoyl)-4R-hydroxysphinganine-1-O-[myo-inositol-1-phosphoryl-6-D-mannopyranosyl-alpha1-2-myo-inositol-1-phosphate]; N-(hexadecanoyl)-4R-hydroxysphinganine-1-phospho-(1′-myo-inositol); N-(hexadecanoyl)-6R-hydroxy-sphing-4E-enine; N-(hexadecanoyl)-deoxysphing-4-enine-1-sulfonate; N-(hexadecanoyl)-eicosasphing-4E-enine-1-phospho-(1′-myo-inositol); N-(hexadecanoyl)-eicosasphinganine; N-(hexadecanoyl)-eicosasphinganine-1-O-[D-mannopyranosyl-alpha1-2-myo-inositol-1-phosphate]; N-(hexadecanoyl)-eicosasphinganine-1-O-[myo-inositol-1-phosphoryl-6-D-mannopyranosyl-alpha1-2-myo-inositol-1-phosphate]; N-(hexadecanoyl)-eicosasphinganine-1-phospho-(1′-myo-inositol); N-(hexadecanoyl)-hexadecasphing-4-enine-1-phosphocholine; N-(hexadecanoyl)-hexadecasphing-4-enine-1-phosphoethanolamine; N-(hexadecanoyl)-hexadecasphinganine; N-(hexadecanoyl)-hexadecasphinganine-1-phosphocholine; N-(hexadecanoyl)-nonadecasphing-4-enine-1-phosphocholine; N-(hexadecanoyl)-sphing-4-enine; N-(hexadecanoyl)-sphing-4-enine-1-(2-aminoethylphosphonate); N-(hexadecanoyl)-sphing-4-enine-1-phosphate; N-(hexadecanoyl)-sphing-4-enine-1-phosphocholine; N-(hexadecanoyl)-sphinganine; N-(hexadecanoyl)-sphinganine-1-O-[D-mannopyranosyl-alpha1-2-myo-inositol-1-phosphate]; N-(hexadecanoyl)-sphinganine-1-phosphate; N-(hexadecanoyl)-sphinganine-1-phospho-(1′-myo-inositol); N-(hexadecanoyl)-sphinganine-1-phosphocholine; N-(hexadecanoyl)-tetradecasphing-4-enine; N-(hexadecanoyl)-tetradecasphing-4-enine-1-phosphoethanolamine; N-(nonacosanoyl)-4E,14Z-sphingadienine; N-(nonacosanoyl)-4R-hydroxysphinganine; N-(nonacosanoyl)-6R-hydroxy-sphing-4E-enine; N-(nonacosanoyl)-sphing-4-enine; N-(nonacosanoyl)-sphinganine; N-(nonadecanoyl)-4E,6E-hexadecasphingadienine-1-phosphoethanolamine; N-(nonadecanoyl)-4E,6E-tetradecasphingadienine-1-phosphoethanolamine; N-(nonadecanoyl)-4R-hydroxysphinganine; N-(nonadecanoyl)-6R-hydroxy-sphing-4E-enine; N-(nonadecanoyl)-eicosasphing-4-enine-1-phosphocholine; N-(nonadecanoyl)-hexadecasphing-4-enine-1-phosphoethanolamine; N-(nonadecanoyl)-sphing-4-enine; N-(nonadecanoyl)-sphing-4-enine-1-phosphocholine; N-(nonadecanoyl)-sphinganine; N-(nonadecanoyl)-tetradecasphing-4-enine-1-phosphoethanolamine; N-(octacosanoyl)-4E,14Z-sphingadienine; N-(octacosanoyl)-4R-hydroxysphinganine; N-(octacosanoyl)-6R-hydroxy-sphing-4E-enine; N-(octacosanoyl)-sphing-4-enine; N-(octacosanoyl)-sphinganine; N-(octadecanoyl)-1-beta-(2′,3′,4′,6′-tetra-O-acetyl-galactosyl)-3-O-acetyl-sphing-4-enine; N-(octadecanoyl)-1-beta-glucosyl-4E,6E-hexadecasphingadienine; N-(octadecanoyl)-1-beta-glucosyl-4E,6E-pentadecasphingadienine; N-(octadecanoyl)-1-beta-glucosyl-4E,6E-tetradecasphingadienine; N-(octadecanoyl)-1-beta-glucosyl-hexadecasphing-4-enine; N-(octadecanoyl)-1-beta-glucosyl-pentadecasphing-4-enine; N-(octadecanoyl)-1-beta-glucosyl-sphing-4-enine; N-(octadecanoyl)-1-beta-glucosyl-sphinganine; N-(octadecanoyl)-1-beta-glucosyl-tetradecasphing-4-enine; N-(octadecanoyl)-1-beta-glucuronosyl-sphing-4-enine; N-(octadecanoyl)-1-beta-lactosyl-hexadecasphinganine; N-(octadecanoyl)-1-beta-lactosyl-sphing-4-enine; N-(octadecanoyl)-1-beta-lactosyl-sphinganine; N-(octadecanoyl)-1-beta-lactosyl-tetradecasphinganine; N-(octadecanoyl)-1-deoxysphing-4-enine; N-(octadecanoyl)-1-deoxysphinganine; N-(octadecanoyl)-4E,14Z-sphingadienine; N-(octadecanoyl)-4E,14Z-sphingadienine-1-phosphocholine; N-(octadecanoyl)-4E,6E-hexadecasphingadienine; N-(octadecanoyl)-4E,6E-hexadecasphingadienine-1-phosphoethanolamine; N-(octadecanoyl)-4E,6E-pentadecasphingadienine-1-phosphoethanolamine; N-(octadecanoyl)-4E,6E-tetradecasphingadienine; N-(octadecanoyl)-4E,6E-tetradecasphingadienine-1-phosphoethanolamine; N-(octadecanoyl)-4E-nonadeca-sphingenine; N-(octadecanoyl)-4R-hydroxy-eicosasphinganine; N-(octadecanoyl)-4R-hydroxyeicosasphinganine-1-O-[D-mannopyranosyl-alpha1-2-myo-inositol-1-phosphate]; N-(octadecanoyl)-4R-hydroxyeicosasphinganine-1-O-[myo-inositol-1-phosphoryl-6-D-mannopyranosyl-alpha1-2-myo-inositol-1-phosphate]; N-(octadecanoyl)-4R-hydroxyeicosasphinganine-1-phospho-(1′-myo-inositol); N-(octadecanoyl)-4R-hydroxysphinganine; N-(octadecanoyl)-4R-hydroxysphinganine-1-O-[D-mannopyranosyl-alpha1-2-myo-inositol-1-phosphate]; N-(octadecanoyl)-4R-hydroxysphinganine-1-O-[myo-inositol-1-phosphoryl-6-D-mannopyranosyl-alpha1-2-myo-inositol-1-phosphate]; N-(octadecanoyl)-4R-hydroxysphinganine-1-phospho-(1′-myo-inositol); N-(octadecanoyl)-6R-hydroxy-sphing-4E-enine; N-(octadecanoyl)-eicosasphinganine; N-(octadecanoyl)-eicosasphinganine-1-O-[D-mannopyranosyl-alpha1-2-myo-inositol-1-phosphate]; N-(octadecanoyl)-eicosasphinganine-1-O-[myo-inositol-1-phosphoryl-6-D-mannopyranosyl-alpha1-2-myo-inositol-1-phosphate]; N-(octadecanoyl)-eicosasphinganine-1-phospho-(1′-myo-inositol); N-(octadecanoyl)-hexadecasphing-4-enine; N-(octadecanoyl)-hexadecasphing-4-enine-1-phosphocholine; N-(octadecanoyl)-hexadecasphing-4-enine-1-phosphoethanolamine; N-(octadecanoyl)-hexadecasphinganine-1-phosphocholine; N-(octadecanoyl)-nonadecasphing-4-enine-1-phosphocholine; N-(octadecanoyl)-pentadecasphing-4-enine-1-phosphoethanolamine; N-(octadecanoyl)-sphing-4-enine; N-(octadecanoyl)-sphing-4-enine-1-phosphate; N-(octadecanoyl)-sphing-4-enine-1-phosphocholine; N-(octadecanoyl)-sphinganine; N-(octadecanoyl)-sphinganine-1-O-[D-mannopyranosyl-alpha1-2-myo-inositol-1-phosphate]; N-(octadecanoyl)-sphinganine-1-O-[myo-inositol-1-phosphoryl-6-D-mannopyranosyl-alpha1-2-myo-inositol-1-phosphate]; N-(octadecanoyl)-sphinganine-1-phospho-(1′-myo-inositol); N-(octadecanoyl)-sphinganine-1-phosphocholine; N-(octadecanoyl)-tetradecasphing-4-enine; N-(octadecanoyl)-tetradecasphing-4-enine-1-phosphoethanolamine; N-(octanoyl)-sphing-4-enine-1-phosphate; N-(pentacosanoyl)-1-beta-glucosyl-heptadecasphing-5E-enine; N-(pentacosanoyl)-4E,14Z-sphingadienine; N-(pentacosanoyl)-4E,14Z-sphingadienine-1-phosphocholine; N-(pentacosanoyl)-4E,6E-tetradecasphingadienine-1-phosphoethanolamine; N-(pentacosanoyl)-4R-hydroxysphinganine; N-(pentacosanoyl)-6R-hydroxy-sphing-4E-enine; N-(pentacosanoyl)-hexadecasphing-4-enine-1-phosphocholine; N-(pentacosanoyl)-hexadecasphinganine-1-phosphocholine; N-(pentacosanoyl)-sphing-4-enine; N-(pentacosanoyl)-sphing-4-enine-1-phosphocholine; N-(pentacosanoyl)-sphinganine; N-(pentacosanoyl)-tetradecasphing-4-enine-1-phosphoethanolamine; N-(pentadecanoyl)-4E,14Z-sphingadienine; N-(pentadecanoyl)-4E,8E-hexacosa-sphingadienine; N-(pentadecanoyl)-4R-hydroxysphinganine; N-(pentadecanoyl)-6R-hydroxy-sphing-4E-enine; N-(pentadecanoyl)-sphing-4-enine; N-(pentadecanoyl)-sphing-4-enine-1-phosphocholine; N-(pentadecanoyl)-sphinganine; N-(pentadecanoyl)-sphinganine-1-phosphocholine; N-(pentatriacontanoyl)-sphing-4-enine; N-(tetracosanoyl)-1-beta-(2′,3′,4′,6′-tetra-O-acetyl-galactosyl)-3-O-acetyl-sphing-4-enine; N-(tetracosanoyl)-1-beta-galactosyl-sphinganine; N-(tetracosanoyl)-1-beta-glucosyl-4E,6E-hexadecasphingadienine; N-(tetracosanoyl)-1-beta-glucosyl-4E,6E-tetradecasphingadienine; N-(tetracosanoyl)-1-beta-glucosyl-hexadecasphing-4-enine; N-(tetracosanoyl)-1-beta-glucosyl-sphing-4-enine; N-(tetracosanoyl)-1-beta-glucosyl-sphinganine; N-(tetracosanoyl)-1-beta-glucosyl-tetradecasphing-4-enine; N-(tetracosanoyl)-1-beta-lactosyl-hexadecasphinganine; N-(tetracosanoyl)-1-beta-lactosyl-sphing-4-enine; N-(tetracosanoyl)-1-beta-lactosyl-sphinganine; N-(tetracosanoyl)-1-deoxysphing-4-enine; N-(tetracosanoyl)-1-deoxysphinganine; N-(tetracosanoyl)-4E,14Z-sphingadienine; N-(tetracosanoyl)-4E,14Z-sphingadienine-1-phosphocholine; N-(tetracosanoyl)-4E,6E-hexadecasphingadienine; N-(tetracosanoyl)-4E,6E-hexadecasphingadienine-1-phosphoethanolamine; N-(tetracosanoyl)-4E,6E-pentadecasphingadienine-1-phosphoethanolamine; N-(tetracosanoyl)-4E,6E-tetradecasphingadienine; N-(tetracosanoyl)-4E,6E-tetradecasphingadienine-1-phosphoethanolamine; N-(tetracosanoyl)-4R-hydroxy-eicosasphinganine; N-(tetracosanoyl)-4R-hydroxyeicosasphinganine-1-O-[D-mannopyranosyl-alpha1-2-myo-inositol-1-phosphate]; N-(tetracosanoyl)-4R-hydroxyeicosasphinganine-1-O-[myo-inositol-1-phosphoryl-6-D-mannopyranosyl-alpha1-2-myo-inositol-1-phosphate]; N-(tetracosanoyl)-4R-hydroxyeicosasphinganine-1-phospho-(1′-myo-inositol); N-(tetracosanoyl)-4R-hydroxysphinganine; N-(tetracosanoyl)-4R-hydroxysphinganine-1-O-[D-mannopyranosyl-alpha1-2-myo-inositol-1-phosphate]; N-(tetracosanoyl)-4R-hydroxysphinganine-1-O-[myo-inositol-1-phosphoryl-6-D-mannopyranosyl-alpha1-2-myo-inositol-1-phosphate]; N-(tetracosanoyl)-4R-hydroxysphinganine-1-phospho-(1′-myo-inositol); N-(tetracosanoyl)-6R-hydroxy-sphing-4E-enine; N-(tetracosanoyl)-eicosasphinganine; N-(tetracosanoyl)-eicosasphinganine-1-O-[D-mannopyranosyl-alpha1-2-myo-inositol-1-phosphate]; N-(tetracosanoyl)-eicosasphinganine-1-O-[myo-inositol-1-phosphoryl-6-D-mannopyranosyl-alpha1-2-myo-inositol-1-phosphate]; N-(tetracosanoyl)-eicosasphinganine-1-phospho-(1′-myo-inositol); N-(tetracosanoyl)-heptadecasphing-4-enine-1-phosphocholine; N-(tetracosanoyl)-hexadecasphing-4-enine; N-(tetracosanoyl)-hexadecasphing-4-enine-1-phosphocholine; N-(tetracosanoyl)-hexadecasphing-4-enine-1-phosphoethanolamine; N-(tetracosanoyl)-hexadecasphinganine; N-(tetracosanoyl)-hexadecasphinganine-1-phosphocholine; N-(tetracosanoyl)-nonadecasphing-4-enine-1-phosphocholine; N-(tetracosanoyl)-pentadecasphing-4-enine; N-(tetracosanoyl)-pentadecasphing-4-enine-1-phosphoethanolamine; N-(tetracosanoyl)-sphing-4-enine; N-(tetracosanoyl)-sphing-4-enine-1-phosphate; N-(tetracosanoyl)-sphing-4-enine-1-phosphocholine; N-(tetracosanoyl)-sphinganine; N-(tetracosanoyl)-sphinganine-1-O-[D-mannopyranosyl-alpha1-2-myo-inositol-1-phosphate]; N-(tetracosanoyl)-sphinganine-1-O-[myo-inositol-1-phosphoryl-6-D-mannopyranosyl-alpha1-2-myo-inositol-1-phosphate]; N-(tetracosanoyl)-sphinganine-1-phospho-(1′-myo-inositol); N-(tetracosanoyl)-sphinganine-1-phosphocholine; N-(tetracosanoyl)-tetradecasphing-4-enine; N-(tetracosanoyl)-tetradecasphing-4-enine-1-phosphoethanolamine; N-(tetradecanoyl)-1-beta-glucosyl-sphing-4-enine; N-(tetradecanoyl)-1-beta-glucosyl-sphinganine; N-(tetradecanoyl)-1-beta-glucuronosyl-hexadecasphinganine; N-(tetradecanoyl)-1-beta-glucuronosyl-sphinganine; N-(tetradecanoyl)-1-beta-lactosyl-sphing-4-enine; N-(tetradecanoyl)-1-beta-lactosyl-sphinganine; N-(tetradecanoyl)-4E,14Z-sphingadienine; N-(tetradecanoyl)-4E,14Z-sphingadienine-1-phosphocholine; N-(tetradecanoyl)-4R-hydroxyeicosasphinganine-1-phospho-(1′-myo-inositol; N-(tetradecanoyl)-4R-hydroxysphinganine; N-(tetradecanoyl)-6R-hydroxy-sphing-4E-enine; N-(tetradecanoyl)-deoxysphing-4-enine-1-sulfonate; N-(tetradecanoyl)-eicosasphing-4E-enine-1-phospho-(1′-myo-inositol); N-(tetradecanoyl)-eicosasphinganine-1-phospho-(1′-myo-inositol); N-(tetradecanoyl)-hexadecasphinganine-1-phosphocholine; N-(tetradecanoyl)-sphing-4E,8E-dienine; N-(tetradecanoyl)-sphing-4E-enine-1-phospho-(1′-myo-inositol); N-(tetradecanoyl)-sphing-4-enine; N-(tetradecanoyl)-sphing-4-enine-1-(2-aminoethylphosphonate); N-(tetradecanoyl)-sphing-4-enine-1-phosphate; N-(tetradecanoyl)-sphing-4-enine-1-phosphocholine; N-(tetradecanoyl)-sphinganine; N-(tetradecanoyl)-sphinganine-1-phosphocholine; N-(tetradecanoyl)-tetradeca-sphinganine; N-(tetratriacontanoyl)-sphing-4-enine; N-(triacontanoyl)-4E,14Z-sphingadienine; N-(triacontanoyl)-4R-hydroxysphinganine; N-(triacontanoyl)-6R-hydroxy-sphing-4E-enine; N-(triacontanoyl)-sphing-4-enine; N-(triacontanoyl)-sphinganine; N-(tricosanoyl)-1-beta-galactosyl-4E,14Z-sphingadienine; N-(tricosanoyl)-1-beta-galactosyl-hexadecasphing-4-enine; N-(tricosanoyl)-1-beta-galactosyl-sphing-4-enine; N-(tricosanoyl)-1beta-glucosyl-4E,14Z-sphingadienine; N-(tricosanoyl)-1-beta-glucosyl-hexadecasphing-4-enine; N-(tricosanoyl)-1-beta-glucosyl-sphing-4-enine; N-(tricosanoyl)-1-beta-lactosyl-hexadecasphinganine; N-(tricosanoyl)-4E,14Z-sphingadienine; N-(tricosanoyl)-4E,14Z-sphingadienine-1-phosphocholine; N-(tricosanoyl)-4E,6E-hexadecasphingadienine-1-phosphoethanolamine; N-(tricosanoyl)-4E,6E-tetradecasphingadienine-1-phosphoethanolamine; N-(tricosanoyl)-4R-hydroxysphinganine; N-(tricosanoyl)-6R-hydroxy-sphing-4E-enine; N-(tricosanoyl)-eicosasphing-4-enine-1-phosphocholine; N-(tricosanoyl)-hexadecasphing-4-enine; N-(tricosanoyl)-hexadecasphing-4-enine-1-phosphocholine; N-(tricosanoyl)-hexadecasphing-4-enine-1-phosphoethanolamine; N-(tricosanoyl)-hexadecasphinganine; N-(tricosanoyl)-hexadecasphinganine-1-phosphocholine; N-(tricosanoyl)-sphing-4-enine; N-(tricosanoyl)-sphing-4-enine-1-phosphocholine; N-(tricosanoyl)-sphinganine; N-(tricosanoyl)-sphinganine-1-phosphocholine; N-(tricosanoyl)-tetradecasphing-4-enine-1-phosphoethanolamine; N-(tridecanoyl)-sphinganine; N-(tridecanoyl)-sphinganine-1-phosphocholine; N-(tritriacontanoyl)-6R-hydroxy-sphing-4E-enine; N-(tritriacontanoyl)-sphing-4-enine; N,N,N-trimethyl-sphing-4E-enine; N,N-dimethylsphing-4-enine; N-acyl-sphing-4-enine; N-acyl-sphing-4-enine-1-phosphocholine; N-acyl-sphinganine; N-acyl-sphinganine-1-phosphate; N-acyl-sphinganine-1-phosphocholine; N-docosanoyl-1-beta-(3′-sulfo)-glucosyl-sphing-4-enine; N-eicosanoyl-1-beta-(3′-sulfo)-glucosyl-sphing-4-enine; NeuAcalpha1-3Galbeta1-4GlcNAcbeta1-3Galalpha1-3(GalNAcbeta1-4)Galbeta1-4Glcbeta-Cer; NeuAcalpha1-3Galbeta1-4GlcNAcbeta1-3Galalpha1-3(GalNAcbeta1-4)Galbeta1-4Glcbeta-Cer(d18:1/16:0); NeuAcalpha1-3Galbeta1-4GlcNAcbeta1-3Galalpha1-3(GalNAcbeta1-4)Galbeta1-4Glcbeta-Cer(d18:1/18:0); NeuAcalpha1-3Galbeta1-4GlcNAcbeta1-3Galalpha1-3(GalNAcbeta1-4)Galbeta1-4Glcbeta-Cer(d18:1/20:0); NeuAcalpha1-3Galbeta1-4GlcNAcbeta1-3Galalpha1-3(GalNAcbeta1-4)Galbeta1-4Glcbeta-Cer(d18:1/22:0); NeuAcalpha1-3Galbeta1-4GlcNAcbeta1-3Galalpha1-3(GalNAcbeta1-4)Galbeta1-4Glcbeta-Cer(d18:1/24:0); NeuAcalpha1-3Galbeta1-4GlcNAcbeta1-3Galalpha1-3(GalNAcbeta1-4)Galbeta1-4Glcbeta-Cer(d18:1/24:1(15Z)); NeuAcalpha1-3Galbeta1-4GlcNAcbeta1-3Galalpha1-3(GalNAcbeta1-4)Galbeta1-4Glcbeta-Cer(d18:1/26:0); NeuAcalpha1-3Galbeta1-4GlcNAcbeta1-3Galalpha1-3(GalNAcbeta1-4)Galbeta1-4Glcbeta-Cer(d18:1/26:1(17Z)); NeuAcalpha1-3Galbeta1-4GlcNAcbeta1-3Galalpha1-3Galbeta1-4Glcbeta-Cer; NeuAcalpha1-3Galbeta1-4GlcNAcbeta1-3Galalpha1-3Galbeta1-4Glcbeta-Cer(d18:1/16:0); NeuAcalpha1-3Galbeta1-4GlcNAcbeta1-3Galalpha1-3Galbeta1-4Glcbeta-Cer(d18:1/18:0); NeuAcalpha1-3Galbeta1-4GlcNAcbeta1-3Galalpha1-3Galbeta1-4Glcbeta-Cer(d18:1/20:0); NeuAcalpha1-3Galbeta1-4GlcNAcbeta1-3Galalpha1-3Galbeta1-4Glcbeta-Cer(d18:1/22:0); NeuAcalpha1-3Galbeta1-4GlcNAcbeta1-3Galalpha1-3Galbeta1-4Glcbeta-Cer(d18:1/24:0); NeuAcalpha1-3Galbeta1-4GlcNAcbeta1-3Galalpha1-3Galbeta1-4Glcbeta-Cer(d18:1/24:1(15Z)); NeuAcalpha1-3Galbeta1-4GlcNAcbeta1-3Galalpha1-3Galbeta1-4Glcbeta-Cer(d18:1/26:0); NeuAcalpha1-3Galbeta1-4GlcNAcbeta1-3Galalpha1-3Galbeta1-4Glcbeta-Cer(d18:1/26:1(17Z)); NeuAcalpha2-3(GalNAcbeta1-4)Galbeta1-4GlcNAcbeta1-6(GalNAcbeta1-3Galalpha1-3)Galbeta1-4Glcbeta-Cer; NeuAcalpha2-3(GalNAcbeta1-4)Galbeta1-4GlcNAcbeta1-6(GalNAcbeta1-3Galalpha1-3)Galbeta1-4Glcbeta-Cer(d18:1/16:0); NeuAcalpha2-3(GalNAcbeta1-4)Galbeta1-4GlcNAcbeta1-6(GalNAcbeta1-3Galalpha1-3)Galbeta1-4Glcbeta-Cer(d18:1/18:0); NeuAcalpha2-3(GalNAcbeta1-4)Galbeta1-4GlcNAcbeta1-6(GalNAcbeta1-3Galalpha1-3)Galbeta1-4Glcbeta-Cer(d18:1/20:0); NeuAcalpha2-3(GalNAcbeta1-4)Galbeta1-4GlcNAcbeta1-6(GalNAcbeta1-3Galalpha1-3)Galbeta1-4Glcbeta-Cer(d18:1/22:0); NeuAcalpha2-3(GalNAcbeta1-4)Galbeta1-4GlcNAcbeta1-6(GalNAcbeta1-3Galalpha1-3)Galbeta1-4Glcbeta-Cer(d18:1/24:0); NeuAcalpha2-3(GalNAcbeta1-4)Galbeta1-4GlcNAcbeta1-6(GalNAcbeta1-3Galalpha1-3)Galbeta1-4Glcbeta-Cer(d18:1/24:1(15Z)); NeuAcalpha2-3(GalNAcbeta1-4)Galbeta1-4GlcNAcbeta1-6(GalNAcbeta1-3Galalpha1-3)Galbeta1-4Glcbeta-Cer(d18:1/26:0); NeuAcalpha2-3(GalNAcbeta1-4)Galbeta1-4GlcNAcbeta1-6(GalNAcbeta1-3Galalpha1-3)Galbeta1-4Glcbeta-Cer(d18:1/26:1(17Z)); NeuAcalpha2-3Galbeta1-3(Fucalpha1-4)GalNAcbeta1-4Galbeta1-4Glcbeta-Cer; NeuAcalpha2-3Galbeta1-3(Fucalpha1-4)GalNAcbeta1-4Galbeta1-4Glcbeta-Cer(d18:1/16:0); NeuAcalpha2-3Galbeta1-3(Fucalpha1-4)GalNAcbeta1-4Galbeta1-4Glcbeta-Cer(d18:1/18:0); NeuAcalpha2-3Galbeta1-3(Fucalpha1-4)GalNAcbeta1-4Galbeta1-4Glcbeta-Cer(d18:1/20:0); NeuAcalpha2-3Galbeta1-3(Fucalpha1-4)GalNAcbeta1-4Galbeta1-4Glcbeta-Cer(d18:1/22:0); NeuAcalpha2-3Galbeta1-3(Fucalpha1-4)GalNAcbeta1-4Galbeta1-4Glcbeta-Cer(d18:1/24:0); NeuAcalpha2-3Galbeta1-3(Fucalpha1-4)GalNAcbeta1-4Galbeta1-4Glcbeta-Cer(d18:1/24:1(15Z)); NeuAcalpha2-3Galbeta1-3(Fucalpha1-4)GalNAcbeta1-4Galbeta1-4Glcbeta-Cer(d18:1/26:0); NeuAcalpha2-3Galbeta1-3(Fucalpha1-4)GalNAcbeta1-4Galbeta1-4Glcbeta-Cer(d18:1/26:1(17Z)); NeuAcalpha2-3Galbeta1-3(Fucalpha1-4)GlcNAcbeta1-3(Fucalpha1-3GlcNAcbeta1-6)Galbeta1-4Glcbeta-Cer; NeuAcalpha2-3Galbeta1-3(Fucalpha1-4)GlcNAcbeta1-3(Fucalpha1-3GlcNAcbeta1-6)Galbeta1-4Glcbeta-Cer(d18:1/16:0); NeuAcalpha2-3Galbeta1-3(Fucalpha1-4)GlcNAcbeta1-3(Fucalpha1-3GlcNAcbeta1-6)Galbeta1-4Glcbeta-Cer(d18:1/18:0); NeuAcalpha2-3Galbeta1-3(Fucalpha1-4)GlcNAcbeta1-3(Fucalpha1-3GlcNAcbeta1-6)Galbeta1-4Glcbeta-Cer(d18:1/20:0); NeuAcalpha2-3Galbeta1-3(Fucalpha1-4)GlcNAcbeta1-3(Fucalpha1-3GlcNAcbeta1-6)Galbeta1-4Glcbeta-Cer(d18:1/22:0); NeuAcalpha2-3Galbeta1-3(Fucalpha1-4)GlcNAcbeta1-3(Fucalpha1-3GlcNAcbeta1-6)Galbeta1-4Glcbeta-Cer(d18:1/24:0); NeuAcalpha2-3Galbeta1-3(Fucalpha1-4)GlcNAcbeta1-3(Fucalpha1-3GlcNAcbeta1-6)Galbeta1-4Glcbeta-Cer(d18:1/24:1(15Z)); NeuAcalpha2-3Galbeta1-3(Fucalpha1-4)GlcNAcbeta1-3(Fucalpha1-3GlcNAcbeta1-6)Galbeta1-4Glcbeta-Cer(d18:1/26:0); NeuAcalpha2-3Galbeta1-3(Fucalpha1-4)GlcNAcbeta1-3(Fucalpha1-3GlcNAcbeta1-6)Galbeta1-4Glcbeta-Cer(d18:1/26:1(17Z)); NeuAcalpha2-3Galbeta1-3(Fucalpha1-4)GlcNAcbeta1-3(Galbeta1-4(Fucalpha1-3)GlcNAcbeta1-6)Galbeta1-4Glcbeta-Cer; NeuAcalpha2-3Galbeta1-3(Fucalpha1-4)GlcNAcbeta1-3(Galbeta1-4(Fucalpha1-3)GlcNAcbeta1-6)Galbeta1-4Glcbeta-Cer(d18:1/16:0); NeuAcalpha2-3Galbeta1-3(Fucalpha1-4)GlcNAcbeta1-3(Galbeta1-4(Fucalpha1-3)GlcNAcbeta1-6)Galbeta1-4Glcbeta-Cer(d18:1/18:0); NeuAcalpha2-3Galbeta1-3(Fucalpha1-4)GlcNAcbeta1-3(Galbeta1-4(Fucalpha1-3)GlcNAcbeta1-6)Galbeta1-4Glcbeta-Cer(d18:1/20:0); NeuAcalpha2-3Galbeta1-3(Fucalpha1-4)GlcNAcbeta1-3(Galbeta1-4(Fucalpha1-3)GlcNAcbeta1-6)Galbeta1-4Glcbeta-Cer(d18:1/22:0); NeuAcalpha2-3Galbeta1-3(Fucalpha1-4)GlcNAcbeta1-3(Galbeta1-4(Fucalpha1-3)GlcNAcbeta1-6)Galbeta1-4Glcbeta-Cer(d18:1/24:0); NeuAcalpha2-3Galbeta1-3(Fucalpha1-4)GlcNAcbeta1-3(Galbeta1-4(Fucalpha1-3)GlcNAcbeta1-6)Galbeta1-4Glcbeta-Cer(d18:1/24:1(15Z)); NeuAcalpha2-3Galbeta1-3(Fucalpha1-4)GlcNAcbeta1-3(Galbeta1-4(Fucalpha1-3)GlcNAcbeta1-6)Galbeta1-4Glcbeta-Cer(d18:1/26:0); NeuAcalpha2-3Galbeta1-3(Fucalpha1-4)GlcNAcbeta1-3(Galbeta1-4(Fucalpha1-3)GlcNAcbeta1-6)Galbeta1-4Glcbeta-Cer(d18:1/26:1(17Z)); NeuAcalpha2-3Galbeta1-3(Fucalpha1-4)GlcNAcbeta1-3(Galbeta1-4GlcNAcbeta1-6)Galbeta1-4Glcbeta-Cer; NeuAcalpha2-3Galbeta1-3(Fucalpha1-4)GlcNAcbeta1-3(Galbeta1-4GlcNAcbeta1-6)Galbeta1-4Glcbeta-Cer(d18:1/16:0); NeuAcalpha2-3Galbeta1-3(Fucalpha1-4)GlcNAcbeta1-3(Galbeta1-4GlcNAcbeta1-6)Galbeta1-4Glcbeta-Cer(d18:1/18:0); NeuAcalpha2-3Galbeta1-3(Fucalpha1-4)GlcNAcbeta1-3(Galbeta1-4GlcNAcbeta1-6)Galbeta1-4Glcbeta-Cer(d18:1/20:0); NeuAcalpha2-3Galbeta1-3(Fucalpha1-4)GlcNAcbeta1-3(Galbeta1-4GlcNAcbeta1-6)Galbeta1-4Glcbeta-Cer(d18:1/22:0); NeuAcalpha2-3Galbeta1-3(Fucalpha1-4)GlcNAcbeta1-3(Galbeta1-4GlcNAcbeta1-6)Galbeta1-4Glcbeta-Cer(d18:1/24:0); NeuAcalpha2-3Galbeta1-3(Fucalpha1-4)GlcNAcbeta1-3(Galbeta1-4GlcNAcbeta1-6)Galbeta1-4Glcbeta-Cer(d18:1/24:1(15Z)); NeuAcalpha2-3Galbeta1-3(Fucalpha1-4)GlcNAcbeta1-3(Galbeta1-4GlcNAcbeta1-6)Galbeta1-4Glcbeta-Cer(d18:1/26:0); NeuAcalpha2-3Galbeta1-3(Fucalpha1-4)GlcNAcbeta1-3(Galbeta1-4GlcNAcbeta1-6)Galbeta1-4Glcbeta-Cer(d18:1/26:1(17Z)); NeuAcalpha2-3Galbeta1-3(Fucalpha1-4)GlcNAcbeta1-3(GlcNAcbeta1-6)Galbeta1-4Glcbeta-Cer; NeuAcalpha2-3Galbeta1-3(Fucalpha1-4)GlcNAcbeta1-3(GlcNAcbeta1-6)Galbeta1-4Glcbeta-Cer(d18:1/16:0); NeuAcalpha2-3Galbeta1-3(Fucalpha1-4)GlcNAcbeta1-3(GlcNAcbeta1-6)Galbeta1-4Glcbeta-Cer(d18:1/18:0); NeuAcalpha2-3Galbeta1-3(Fucalpha1-4)GlcNAcbeta1-3(GlcNAcbeta1-6)Galbeta1-4Glcbeta-Cer(d18:1/20:0); NeuAcalpha2-3Galbeta1-3(Fucalpha1-4)GlcNAcbeta1-3(GlcNAcbeta1-6)Galbeta1-4Glcbeta-Cer(d18:1/22:0); NeuAcalpha2-3Galbeta1-3(Fucalpha1-4)GlcNAcbeta1-3(GlcNAcbeta1-6)Galbeta1-4Glcbeta-Cer(d18:1/24:0); NeuAcalpha2-3Galbeta1-3(Fucalpha1-4)GlcNAcbeta1-3(GlcNAcbeta1-6)Galbeta1-4Glcbeta-Cer(d18:1/24:1(15Z)); NeuAcalpha2-3Galbeta1-3(Fucalpha1-4)GlcNAcbeta1-3(GlcNAcbeta1-6)Galbeta1-4Glcbeta-Cer(d18:1/26:0); NeuAcalpha2-3Galbeta1-3(Fucalpha1-4)GlcNAcbeta1-3(GlcNAcbeta1-6)Galbeta1-4Glcbeta-Cer(d18:1/26:1(17Z)); NeuAcalpha2-3Galbeta1-3(Fucalpha1-4)GlcNAcbeta1-3Galbeta1-4(Fucalpha1-3)GlcNAcbeta1-3Galbeta1-4Glcbeta-Cer; NeuAcalpha2-3Galbeta1-3(Fucalpha1-4)GlcNAcbeta1-3Galbeta1-4(Fucalpha1-3)GlcNAcbeta1-3Galbeta1-4Glcbeta-Cer(d18:1/16:0); NeuAcalpha2-3Galbeta1-3(Fucalpha1-4)GlcNAcbeta1-3Galbeta1-4(Fucalpha1-3)GlcNAcbeta1-3Galbeta1-4Glcbeta-Cer(d18:1/18:0); NeuAcalpha2-3Galbeta1-3(Fucalpha1-4)GlcNAcbeta1-3Galbeta1-4(Fucalpha1-3)GlcNAcbeta1-3Galbeta1-4Glcbeta-Cer(d18:1/20:0); NeuAcalpha2-3Galbeta1-3(Fucalpha1-4)GlcNAcbeta1-3Galbeta1-4(Fucalpha1-3)GlcNAcbeta1-3Galbeta1-4Glcbeta-Cer(d18:1/22:0); NeuAcalpha2-3Galbeta1-3(Fucalpha1-4)GlcNAcbeta1-3Galbeta1-4(Fucalpha1-3)GlcNAcbeta1-3Galbeta1-4Glcbeta-Cer(d18:1/24:0); NeuAcalpha2-3Galbeta1-3(Fucalpha1-4)GlcNAcbeta1-3Galbeta1-4(Fucalpha1-3)GlcNAcbeta1-3Galbeta1-4Glcbeta-Cer(d18:1/24:1(15Z)); NeuAcalpha2-3Galbeta1-3(Fucalpha1-4)GlcNAcbeta1-3Galbeta1-4(Fucalpha1-3)GlcNAcbeta1-3Galbeta1-4Glcbeta-Cer(d18:1/26:0); NeuAcalpha2-3Galbeta1-3(Fucalpha1-4)GlcNAcbeta1-3Galbeta1-4(Fucalpha1-3)GlcNAcbeta1-3Galbeta1-4Glcbeta-Cer(d18:1/26:1(17Z)); NeuAcalpha2-3Galbeta1-3(Fucalpha1-4)GlcNAcbeta1-3Galbeta1-4Glcbeta-Cer; NeuAcalpha2-3Galbeta1-3(Fucalpha1-4)GlcNAcbeta1-3Galbeta1-4Glcbeta-Cer(d18:1/16:0); NeuAcalpha2-3Galbeta1-3(Fucalpha1-4)GlcNAcbeta1-3Galbeta1-4Glcbeta-Cer(d18:1/18:0); NeuAcalpha2-3Galbeta1-3(Fucalpha1-4)GlcNAcbeta1-3Galbeta1-4Glcbeta-Cer(d18:1/20:0); NeuAcalpha2-3Galbeta1-3(Fucalpha1-4)GlcNAcbeta1-3Galbeta1-4Glcbeta-Cer(d18:1/22:0); NeuAcalpha2-3Galbeta1-3(Fucalpha1-4)GlcNAcbeta1-3Galbeta1-4Glcbeta-Cer(d18:1/24:0); NeuAcalpha2-3Galbeta1-3(Fucalpha1-4)GlcNAcbeta1-3Galbeta1-4Glcbeta-Cer(d18:1/24:1(15Z)); NeuAcalpha2-3Galbeta1-3(Fucalpha1-4)GlcNAcbeta1-3Galbeta1-4Glcbeta-Cer(d18:1/26:0); NeuAcalpha2-3Galbeta1-3(Fucalpha1-4)GlcNAcbeta1-3Galbeta1-4Glcbeta-Cer(d18:1/26:1(17Z)); NeuAcalpha2-3Galbeta1-3(Fucalpha1-4)GlcNAcbeta1-3Galbeta1-4GlcNAcbeta1-3Galbeta1-4Glcbeta-Cer; NeuAcalpha2-3Galbeta1-3(Fucalpha1-4)GlcNAcbeta1-3Galbeta1-4GlcNAcbeta1-3Galbeta1-4Glcbeta-Cer(d18:1/16:0); NeuAcalpha2-3Galbeta1-3(Fucalpha1-4)GlcNAcbeta1-3Galbeta1-4GlcNAcbeta1-3Galbeta1-4Glcbeta-Cer(d18:1/18:0); NeuAcalpha2-3Galbeta1-3(Fucalpha1-4)GlcNAcbeta1-3Galbeta1-4GlcNAcbeta1-3Galbeta1-4Glcbeta-Cer(d18:1/20:0); NeuAcalpha2-3Galbeta1-3(Fucalpha1-4)GlcNAcbeta1-3Galbeta1-4GlcNAcbeta1-3Galbeta1-4Glcbeta-Cer(d18:1/22:0); NeuAcalpha2-3Galbeta1-3(Fucalpha1-4)GlcNAcbeta1-3Galbeta1-4GlcNAcbeta1-3Galbeta1-4Glcbeta-Cer(d18:1/24:0); NeuAcalpha2-3Galbeta1-3(Fucalpha1-4)GlcNAcbeta1-3Galbeta1-4GlcNAcbeta1-3Galbeta1-4Glcbeta-Cer(d18:1/24:1(15Z)); NeuAcalpha2-3Galbeta1-3(Fucalpha1-4)GlcNAcbeta1-3Galbeta1-4GlcNAcbeta1-3Galbeta1-4Glcbeta-Cer(d18:1/26:0); NeuAcalpha2-3Galbeta1-3(Fucalpha1-4)GlcNAcbeta1-3Galbeta1-4GlcNAcbeta1-3Galbeta1-4Glcbeta-Cer(d18:1/26:1(17Z)); NeuAcalpha2-3Galbeta1-3(NeuAcalpha2-6)(Fucalpha1-4)GlcNAcbeta1-3Galbeta1-4Glcbeta-Cer; NeuAcalpha2-3Galbeta1-3(NeuAcalpha2-6)(Fucalpha1-4)GlcNAcbeta1-3Galbeta1-4Glcbeta-Cer(d18:1/16:0); NeuAcalpha2-3Galbeta1-3(NeuAcalpha2-6)(Fucalpha1-4)GlcNAcbeta1-3Galbeta1-4Glcbeta-Cer(d18:1/18:0); NeuAcalpha2-3Galbeta1-3(NeuAcalpha2-6)(Fucalpha1-4)GlcNAcbeta1-3Galbeta1-4Glcbeta-Cer(d18:1/20:0); NeuAcalpha2-3Galbeta1-3(NeuAcalpha2-6)(Fucalpha1-4)GlcNAcbeta1-3Galbeta1-4Glcbeta-Cer(d18:1/22:0); NeuAcalpha2-3Galbeta1-3(NeuAcalpha2-6)(Fucalpha1-4)GlcNAcbeta1-3Galbeta1-4Glcbeta-Cer(d18:1/24:0); NeuAcalpha2-3Galbeta1-3(NeuAcalpha2-6)(Fucalpha1-4)GlcNAcbeta1-3Galbeta1-4Glcbeta-Cer(d18:1/24:1(15Z)); NeuAcalpha2-3Galbeta1-3(NeuAcalpha2-6)(Fucalpha1-4)GlcNAcbeta1-3Galbeta1-4Glcbeta-Cer(d18:1/26:0); NeuAcalpha2-3Galbeta1-3(NeuAcalpha2-6)(Fucalpha1-4)GlcNAcbeta1-3Galbeta1-4Glcbeta-Cer(d18:1/26:1(17Z)); NeuAcalpha2-3Galbeta1-3(NeuAcalpha2-6)GalNAcbeta1-3Galalpha1-4Galbeta1-4Glcbeta-Cer; NeuAcalpha2-3Galbeta1-3(NeuAcalpha2-6)GalNAcbeta1-3Galalpha1-4Galbeta1-4Glcbeta-Cer(d18:1/16:0); NeuAcalpha2-3Galbeta1-3(NeuAcalpha2-6)GalNAcbeta1-3Galalpha1-4Galbeta1-4Glcbeta-Cer(d18:1/18:0); NeuAcalpha2-3Galbeta1-3(NeuAcalpha2-6)GalNAcbeta1-3Galalpha1-4Galbeta1-4Glcbeta-Cer(d18:1/20:0); NeuAcalpha2-3Galbeta1-3(NeuAcalpha2-6)GalNAcbeta1-3Galalpha1-4Galbeta1-4Glcbeta-Cer(d18:1/22:0); NeuAcalpha2-3Galbeta1-3(NeuAcalpha2-6)GalNAcbeta1-3Galalpha1-4Galbeta1-4Glcbeta-Cer(d18:1/24:0); NeuAcalpha2-3Galbeta1-3(NeuAcalpha2-6)GalNAcbeta1-3Galalpha1-4Galbeta1-4Glcbeta-Cer(d18:1/24:1(15Z)); NeuAcalpha2-3Galbeta1-3(NeuAcalpha2-6)GalNAcbeta1-3Galalpha1-4Galbeta1-4Glcbeta-Cer(d18:1/26:0); NeuAcalpha2-3Galbeta1-3(NeuAcalpha2-6)GalNAcbeta1-3Galalpha1-4Galbeta1-4Glcbeta-Cer(d18:1/26:1(17Z)); NeuAcalpha2-3Galbeta1-3(NeuAcalpha2-6)GalNAcbeta1-4(NeuAcalpha2-3)Galbeta1-4Glcbeta-Cer; NeuAcalpha2-3Galbeta1-3(NeuAcalpha2-6)GalNAcbeta1-4(NeuAcalpha2-3)Galbeta1-4Glcbeta-Cer(d18:1/16:0); NeuAcalpha2-3Galbeta1-3(NeuAcalpha2-6)GalNAcbeta1-4(NeuAcalpha2-3)Galbeta1-4Glcbeta-Cer(d18:1/18:0); NeuAcalpha2-3Galbeta1-3(NeuAcalpha2-6)GalNAcbeta1-4(NeuAcalpha2-3)Galbeta1-4Glcbeta-Cer(d18:1/20:0); NeuAcalpha2-3Galbeta1-3(NeuAcalpha2-6)GalNAcbeta1-4(NeuAcalpha2-3)Galbeta1-4Glcbeta-Cer(d18:1/22:0); NeuAcalpha2-3Galbeta1-3(NeuAcalpha2-6)GalNAcbeta1-4(NeuAcalpha2-3)Galbeta1-4Glcbeta-Cer(d18:1/24:0); NeuAcalpha2-3Galbeta1-3(NeuAcalpha2-6)GalNAcbeta1-4(NeuAcalpha2-3)Galbeta1-4Glcbeta-Cer(d18:1/24:1(15Z)); NeuAcalpha2-3Galbeta1-3(NeuAcalpha2-6)GalNAcbeta1-4(NeuAcalpha2-3)Galbeta1-4Glcbeta-Cer(d18:1/26:0); NeuAcalpha2-3Galbeta1-3(NeuAcalpha2-6)GalNAcbeta1-4(NeuAcalpha2-3)Galbeta1-4Glcbeta-Cer(d18:1/26:1(17Z)); NeuAcalpha2-3Galbeta1-3(NeuAcalpha2-6)GalNAcbeta1-4(NeuAcalpha2-8NeuAcalpha2-3)Galbeta1-4Glcbeta-Cer; NeuAcalpha2-3Galbeta1-3(NeuAcalpha2-6)GalNAcbeta1-4(NeuAcalpha2-8NeuAcalpha2-3)Galbeta1-4Glcbeta-Cer(d18:1/16:0); NeuAcalpha2-3Galbeta1-3(NeuAcalpha2-6)GalNAcbeta1-4(NeuAcalpha2-8NeuAcalpha2-3)Galbeta1-4Glcbeta-Cer(d18:1/18:0); NeuAcalpha2-3Galbeta1-3(NeuAcalpha2-6)GalNAcbeta1-4(NeuAcalpha2-8NeuAcalpha2-3)Galbeta1-4Glcbeta-Cer(d18:1/20:0); NeuAcalpha2-3Galbeta1-3(NeuAcalpha2-6)GalNAcbeta1-4(NeuAcalpha2-8NeuAcalpha2-3)Galbeta1-4Glcbeta-Cer(d18:1/22:0); NeuAcalpha2-3Galbeta1-3(NeuAcalpha2-6)GalNAcbeta1-4(NeuAcalpha2-8NeuAcalpha2-3)Galbeta1-4Glcbeta-Cer(d18:1/24:0); NeuAcalpha2-3Galbeta1-3(NeuAcalpha2-6)GalNAcbeta1-4(NeuAcalpha2-8NeuAcalpha2-3)Galbeta1-4Glcbeta-Cer(d18:1/24:1(15Z)); NeuAcalpha2-3Galbeta1-3(NeuAcalpha2-6)GalNAcbeta1-4(NeuAcalpha2-8NeuAcalpha2-3)Galbeta1-4Glcbeta-Cer(d18:1/26:0); NeuAcalpha2-3Galbeta1-3(NeuAcalpha2-6)GalNAcbeta1-4(NeuAcalpha2-8NeuAcalpha2-3)Galbeta1-4Glcbeta-Cer(d18:1/26:1(17Z)); NeuAcalpha2-3Galbeta1-3(NeuAcalpha2-6)GalNAcbeta1-4Galbeta1-4Glcbeta-Cer; NeuAcalpha2-3Galbeta1-3(NeuAcalpha2-6)GalNAcbeta1-4Galbeta1-4Glcbeta-Cer(d18:1/16:0); NeuAcalpha2-3Galbeta1-3(NeuAcalpha2-6)GalNAcbeta1-4Galbeta1-4Glcbeta-Cer(d18:1/18:0); NeuAcalpha2-3Galbeta1-3(NeuAcalpha2-6)GalNAcbeta1-4Galbeta1-4Glcbeta-Cer(d18:1/20:0); NeuAcalpha2-3Galbeta1-3(NeuAcalpha2-6)GalNAcbeta1-4Galbeta1-4Glcbeta-Cer(d18:1/22:0); NeuAcalpha2-3Galbeta1-3(NeuAcalpha2-6)GalNAcbeta1-4Galbeta1-4Glcbeta-Cer(d18:1/24:0); NeuAcalpha2-3Galbeta1-3(NeuAcalpha2-6)GalNAcbeta1-4Galbeta1-4Glcbeta-Cer(d18:1/24:1(15Z)); NeuAcalpha2-3Galbeta1-3(NeuAcalpha2-6)GalNAcbeta1-4Galbeta1-4Glcbeta-Cer(d18:1/26:0); NeuAcalpha2-3Galbeta1-3(NeuAcalpha2-6)GalNAcbeta1-4Galbeta1-4Glcbeta-Cer(d18:1/26:1(17Z)); NeuAcalpha2-3Galbeta1-3(NeuAcalpha2-6)GlcNAcbeta1-3Galbeta1-4Glcbeta-Cer; NeuAcalpha2-3Galbeta1-3(NeuAcalpha2-6)GlcNAcbeta1-3Galbeta1-4Glcbeta-Cer(d18:1/16:0); NeuAcalpha2-3Galbeta1-3(NeuAcalpha2-6)GlcNAcbeta1-3Galbeta1-4Glcbeta-Cer(d18:1/18:0); NeuAcalpha2-3Galbeta1-3(NeuAcalpha2-6)GlcNAcbeta1-3Galbeta1-4Glcbeta-Cer(d18:1/20:0); NeuAcalpha2-3Galbeta1-3(NeuAcalpha2-6)GlcNAcbeta1-3Galbeta1-4Glcbeta-Cer(d18:1/22:0); NeuAcalpha2-3Galbeta1-3(NeuAcalpha2-6)GlcNAcbeta1-3Galbeta1-4Glcbeta-Cer(d18:1/24:0); NeuAcalpha2-3Galbeta1-3(NeuAcalpha2-6)GlcNAcbeta1-3Galbeta1-4Glcbeta-Cer(d18:1/24:1(15Z)); NeuAcalpha2-3Galbeta1-3(NeuAcalpha2-6)GlcNAcbeta1-3Galbeta1-4Glcbeta-Cer(d18:1/26:0); NeuAcalpha2-3Galbeta1-3(NeuAcalpha2-6)GlcNAcbeta1-3Galbeta1-4Glcbeta-Cer(d18:1/26:1(17Z)); NeuAcalpha2-3Galbeta1-3(NeuAcalpha2-8NeuAcalpha2-6)GalNAcbeta1-4Galbeta1-4Glcbeta-Cer; NeuAcalpha2-3Galbeta1-3(NeuAcalpha2-8NeuAcalpha2-6)GalNAcbeta1-4Galbeta1-4Glcbeta-Cer(d18:1/16:0); NeuAcalpha2-3Galbeta1-3(NeuAcalpha2-8NeuAcalpha2-6)GalNAcbeta1-4Galbeta1-4Glcbeta-Cer(d18:1/18:0); NeuAcalpha2-3Galbeta1-3(NeuAcalpha2-8NeuAcalpha2-6)GalNAcbeta1-4Galbeta1-4Glcbeta-Cer(d18:1/20:0); NeuAcalpha2-3Galbeta1-3(NeuAcalpha2-8NeuAcalpha2-6)GalNAcbeta1-4Galbeta1-4Glcbeta-Cer(d18:1/22:0); NeuAcalpha2-3Galbeta1-3(NeuAcalpha2-8NeuAcalpha2-6)GalNAcbeta1-4Galbeta1-4Glcbeta-Cer(d18:1/24:0); NeuAcalpha2-3Galbeta1-3(NeuAcalpha2-8NeuAcalpha2-6)GalNAcbeta1-4Galbeta1-4Glcbeta-Cer(d18:1/24:1(15Z)); NeuAcalpha2-3Galbeta1-3(NeuAcalpha2-8NeuAcalpha2-6)GalNAcbeta1-4Galbeta1-4Glcbeta-Cer(d18:1/26:0); NeuAcalpha2-3Galbeta1-3(NeuAcalpha2-8NeuAcalpha2-6)GalNAcbeta1-4Galbeta1-4Glcbeta-Cer(d18:1/26:1(17Z)); NeuAcalpha2-3Galbeta1-3Galbeta1-3GlcNAcbeta1-3Galbeta1-4Glcbeta-Cer; NeuAcalpha2-3Galbeta1-3Galbeta1-3GlcNAcbeta1-3Galbeta1-4Glcbeta-Cer(d18:1/16:0); NeuAcalpha2-3Galbeta1-3Galbeta1-3GlcNAcbeta1-3Galbeta1-4Glcbeta-Cer(d18:1/18:0); NeuAcalpha2-3Galbeta1-3Galbeta1-3GlcNAcbeta1-3Galbeta1-4Glcbeta-Cer(d18:1/20:0); NeuAcalpha2-3Galbeta1-3Galbeta1-3GlcNAcbeta1-3Galbeta1-4Glcbeta-Cer(d18:1/22:0); NeuAcalpha2-3Galbeta1-3Galbeta1-3GlcNAcbeta1-3Galbeta1-4Glcbeta-Cer(d18:1/24:0); NeuAcalpha2-3Galbeta1-3Galbeta1-3GlcNAcbeta1-3Galbeta1-4Glcbeta-Cer(d18:1/24:1(15Z)); NeuAcalpha2-3Galbeta1-3Galbeta1-3GlcNAcbeta1-3Galbeta1-4Glcbeta-Cer(d18:1/26:0); NeuAcalpha2-3Galbeta1-3Galbeta1-3GlcNAcbeta1-3Galbeta1-4Glcbeta-Cer(d18:1/26:1(17Z)); NeuAcalpha2-3Galbeta1-3Galbeta1-4GlcNAcbeta1-3Galbeta1-4Glcbeta-Cer; NeuAcalpha2-3Galbeta1-3Galbeta1-4GlcNAcbeta1-3Galbeta1-4Glcbeta-Cer(d18:1/16:0); NeuAcalpha2-3Galbeta1-3Galbeta1-4GlcNAcbeta1-3Galbeta1-4Glcbeta-Cer(d18:1/18:0); NeuAcalpha2-3Galbeta1-3Galbeta1-4GlcNAcbeta1-3Galbeta1-4Glcbeta-Cer(d18:1/20:0); NeuAcalpha2-3Galbeta1-3Galbeta1-4GlcNAcbeta1-3Galbeta1-4Glcbeta-Cer(d18:1/22:0); NeuAcalpha2-3Galbeta1-3Galbeta1-4GlcNAcbeta1-3Galbeta1-4Glcbeta-Cer(d18:1/24:0); NeuAcalpha2-3Galbeta1-3Galbeta1-4GlcNAcbeta1-3Galbeta1-4Glcbeta-Cer(d18:1/24:1(15Z)); NeuAcalpha2-3Galbeta1-3Galbeta1-4GlcNAcbeta1-3Galbeta1-4Glcbeta-Cer(d18:1/26:0); NeuAcalpha2-3Galbeta1-3Galbeta1-4GlcNAcbeta1-3Galbeta1-4Glcbeta-Cer(d18:1/26:1(17Z)); NeuAcalpha2-3Galbeta1-3GalNAcalpha1-3(Fucalpha1-2)Galbeta1-4GlcNAcbeta1-3Galbeta1-4Glcbeta-Cer; NeuAcalpha2-3Galbeta1-3GalNAcalpha1-3(Fucalpha1-2)Galbeta1-4GlcNAcbeta1-3Galbeta1-4Glcbeta-Cer(d18:1/16:0); NeuAcalpha2-3Galbeta1-3GalNAcalpha1-3(Fucalpha1-2)Galbeta1-4GlcNAcbeta1-3Galbeta1-4Glcbeta-Cer(d18:1/18:0); NeuAcalpha2-3Galbeta1-3GalNAcalpha1-3(Fucalpha1-2)Galbeta1-4GlcNAcbeta1-3Galbeta1-4Glcbeta-Cer(d18:1/20:0); NeuAcalpha2-3Galbeta1-3GalNAcalpha1-3(Fucalpha1-2)Galbeta1-4GlcNAcbeta1-3Galbeta1-4Glcbeta-Cer(d18:1/22:0); NeuAcalpha2-3Galbeta1-3GalNAcalpha1-3(Fucalpha1-2)Galbeta1-4GlcNAcbeta1-3Galbeta1-4Glcbeta-Cer(d18:1/24:0); NeuAcalpha2-3Galbeta1-3GalNAcalpha1-3(Fucalpha1-2)Galbeta1-4GlcNAcbeta1-3Galbeta1-4Glcbeta-Cer(d18:1/24:1(15Z)); NeuAcalpha2-3Galbeta1-3GalNAcalpha1-3(Fucalpha1-2)Galbeta1-4GlcNAcbeta1-3Galbeta1-4Glcbeta-Cer(d18:1/26:0); NeuAcalpha2-3Galbeta1-3GalNAcalpha1-3(Fucalpha1-2)Galbeta1-4GlcNAcbeta1-3Galbeta1-4Glcbeta-Cer(d18:1/26:1(17Z)); NeuAcalpha2-3Galbeta1-3GalNAcbeta1-3Galalpha1-3Galbeta1-4Glcbeta-Cer; NeuAcalpha2-3Galbeta1-3GalNAcbeta1-3Galalpha1-3Galbeta1-4Glcbeta-Cer(d18:1/16:0); NeuAcalpha2-3Galbeta1-3GalNAcbeta1-3Galalpha1-3Galbeta1-4Glcbeta-Cer(d18:1/18:0); NeuAcalpha2-3Galbeta1-3GalNAcbeta1-3Galalpha1-3Galbeta1-4Glcbeta-Cer(d18:1/20:0); NeuAcalpha2-3Galbeta1-3GalNAcbeta1-3Galalpha1-3Galbeta1-4Glcbeta-Cer(d18:1/22:0); NeuAcalpha2-3Galbeta1-3GalNAcbeta1-3Galalpha1-3Galbeta1-4Glcbeta-Cer(d18:1/24:0); NeuAcalpha2-3Galbeta1-3GalNAcbeta1-3Galalpha1-3Galbeta1-4Glcbeta-Cer(d18:1/24:1(15Z)); NeuAcalpha2-3Galbeta1-3GalNAcbeta1-3Galalpha1-3Galbeta1-4Glcbeta-Cer(d18:1/26:0); NeuAcalpha2-3Galbeta1-3GalNAcbeta1-3Galalpha1-3Galbeta1-4Glcbeta-Cer(d18:1/26:1(17Z)); NeuAcalpha2-3Galbeta1-3GalNAcbeta1-3Galalpha1-4Galbeta1-4Glcbeta-Cer; NeuAcalpha2-3Galbeta1-3GalNAcbeta1-3Galalpha1-4Galbeta1-4Glcbeta-Cer(d18:1/16:0); NeuAcalpha2-3Galbeta1-3GalNAcbeta1-3Galalpha1-4Galbeta1-4Glcbeta-Cer(d18:1/18:0); NeuAcalpha2-3Galbeta1-3GalNAcbeta1-3Galalpha1-4Galbeta1-4Glcbeta-Cer(d18:1/20:0); NeuAcalpha2-3Galbeta1-3GalNAcbeta1-3Galalpha1-4Galbeta1-4Glcbeta-Cer(d18:1/22:0); NeuAcalpha2-3Galbeta1-3GalNAcbeta1-3Galalpha1-4Galbeta1-4Glcbeta-Cer(d18:1/24:0); NeuAcalpha2-3Galbeta1-3GalNAcbeta1-3Galalpha1-4Galbeta1-4Glcbeta-Cer(d18:1/24:1(15Z)); NeuAcalpha2-3Galbeta1-3GalNAcbeta1-3Galalpha1-4Galbeta1-4Glcbeta-Cer(d18:1/26:0); NeuAcalpha2-3Galbeta1-3GalNAcbeta1-3Galalpha1-4Galbeta1-4Glcbeta-Cer(d18:1/26:1(17Z)); NeuAcalpha2-3Galbeta1-3GalNAcbeta1-4(9-OAc-NeuAcalpha2-8NeuAcalpha2-3)Galbeta1-4Glcbeta-Cer; NeuAcalpha2-3Galbeta1-3GalNAcbeta1-4(9-OAc-NeuAcalpha2-8NeuAcalpha2-3)Galbeta1-4Glcbeta-Cer(d18:1/16:0); NeuAcalpha2-3Galbeta1-3GalNAcbeta1-4(9-OAc-NeuAcalpha2-8NeuAcalpha2-3)Galbeta1-4Glcbeta-Cer(d18:1/18:0); NeuAcalpha2-3Galbeta1-3GalNAcbeta1-4(9-OAc-NeuAcalpha2-8NeuAcalpha2-3)Galbeta1-4Glcbeta-Cer(d18:1/20:0); NeuAcalpha2-3Galbeta1-3GalNAcbeta1-4(9-OAc-NeuAcalpha2-8NeuAcalpha2-3)Galbeta1-4Glcbeta-Cer(d18:1/22:0); NeuAcalpha2-3Galbeta1-3GalNAcbeta1-4(9-OAc-NeuAcalpha2-8NeuAcalpha2-3)Galbeta1-4Glcbeta-Cer(d18:1/24:0); NeuAcalpha2-3Galbeta1-3GalNAcbeta1-4(9-OAc-NeuAcalpha2-8NeuAcalpha2-3)Galbeta1-4Glcbeta-Cer(d18:1/24:1(15Z)); NeuAcalpha2-3Galbeta1-3GalNAcbeta1-4(9-OAc-NeuAcalpha2-8NeuAcalpha2-3)Galbeta1-4Glcbeta-Cer(d18:1/26:0); NeuAcalpha2-3Galbeta1-3GalNAcbeta1-4(9-OAc-NeuAcalpha2-8NeuAcalpha2-3)Galbeta1-4Glcbeta-Cer(d18:1/26:1(17Z)); NeuAcalpha2-3Galbeta1-3GalNAcbeta1-4(KDNalpha2-3)Galbeta1-4Glcbeta-Cer; NeuAcalpha2-3Galbeta1-3GalNAcbeta1-4(KDNalpha2-3)Galbeta1-4Glcbeta-Cer(d18:1/16:0); NeuAcalpha2-3Galbeta1-3GalNAcbeta1-4(KDNalpha2-3)Galbeta1-4Glcbeta-Cer(d18:1/18:0); NeuAcalpha2-3Galbeta1-3GalNAcbeta1-4(KDNalpha2-3)Galbeta1-4Glcbeta-Cer(d18:1/20:0); NeuAcalpha2-3Galbeta1-3GalNAcbeta1-4(KDNalpha2-3)Galbeta1-4Glcbeta-Cer(d18:1/22:0); NeuAcalpha2-3Galbeta1-3GalNAcbeta1-4(KDNalpha2-3)Galbeta1-4Glcbeta-Cer(d18:1/24:0); NeuAcalpha2-3Galbeta1-3GalNAcbeta1-4(KDNalpha2-3)Galbeta1-4Glcbeta-Cer(d18:1/24:1(15Z)); NeuAcalpha2-3Galbeta1-3GalNAcbeta1-4(KDNalpha2-3)Galbeta1-4Glcbeta-Cer(d18:1/26:0); NeuAcalpha2-3Galbeta1-3GalNAcbeta1-4(KDNalpha2-3)Galbeta1-4Glcbeta-Cer(d18:1/26:1(17Z)); NeuAcalpha2-3Galbeta1-3GalNAcbeta1-4(NeuAcalpha2-3)Galbeta1-4Glcbeta-Cer; NeuAcalpha2-3Galbeta1-3GalNAcbeta1-4(NeuAcalpha2-3)Galbeta1-4Glcbeta-Cer(d18:1/16:0); NeuAcalpha2-3Galbeta1-3GalNAcbeta1-4(NeuAcalpha2-3)Galbeta1-4Glcbeta-Cer(d18:1/18:0); NeuAcalpha2-3Galbeta1-3GalNAcbeta1-4(NeuAcalpha2-3)Galbeta1-4Glcbeta-Cer(d18:1/20:0); NeuAcalpha2-3Galbeta1-3GalNAcbeta1-4(NeuAcalpha2-3)Galbeta1-4Glcbeta-Cer(d18:1/22:0); NeuAcalpha2-3Galbeta1-3GalNAcbeta1-4(NeuAcalpha2-3)Galbeta1-4Glcbeta-Cer(d18:1/24:0); NeuAcalpha2-3Galbeta1-3GalNAcbeta1-4(NeuAcalpha2-3)Galbeta1-4Glcbeta-Cer(d18:1/24:1(15Z)); NeuAcalpha2-3Galbeta1-3GalNAcbeta1-4(NeuAcalpha2-3)Galbeta1-4Glcbeta-Cer(d18:1/26:0); NeuAcalpha2-3Galbeta1-3GalNAcbeta1-4(NeuAcalpha2-3)Galbeta1-4Glcbeta-Cer(d18:1/26:1(17Z)); NeuAcalpha2-3Galbeta1-3GalNAcbeta1-4(NeuAcalpha2-8NeuAcalpha2-3)Galbeta1-4Glcbeta-Cer; NeuAcalpha2-3Galbeta1-3GalNAcbeta1-4(NeuAcalpha2-8NeuAcalpha2-3)Galbeta1-4Glcbeta-Cer(d18:1/16:0); NeuAcalpha2-3Galbeta1-3GalNAcbeta1-4(NeuAcalpha2-8NeuAcalpha2-3)Galbeta1-4Glcbeta-Cer(d18:1/18:0); NeuAcalpha2-3Galbeta1-3GalNAcbeta1-4(NeuAcalpha2-8NeuAcalpha2-3)Galbeta1-4Glcbeta-Cer(d18:1/20:0); NeuAcalpha2-3Galbeta1-3GalNAcbeta1-4(NeuAcalpha2-8NeuAcalpha2-3)Galbeta1-4Glcbeta-Cer(d18:1/22:0); NeuAcalpha2-3Galbeta1-3GalNAcbeta1-4(NeuAcalpha2-8NeuAcalpha2-3)Galbeta1-4Glcbeta-Cer(d18:1/24:0); NeuAcalpha2-3Galbeta1-3GalNAcbeta1-4(NeuAcalpha2-8NeuAcalpha2-3)Galbeta1-4Glcbeta-Cer(d18:1/24:1(15Z)); NeuAcalpha2-3Galbeta1-3GalNAcbeta1-4(NeuAcalpha2-8NeuAcalpha2-3)Galbeta1-4Glcbeta-Cer(d18:1/26:0); NeuAcalpha2-3Galbeta1-3GalNAcbeta1-4(NeuAcalpha2-8NeuAcalpha2-3)Galbeta1-4Glcbeta-Cer(d18:1/26:1(17Z)); NeuAcalpha2-3Galbeta1-3GalNAcbeta1-4(NeuAcalpha2-8NeuAcalpha2-8NeuAcalpha2-3)Galbeta1-4Glcbeta-Cer; NeuAcalpha2-3Galbeta1-3GalNAcbeta1-4(NeuAcalpha2-8NeuAcalpha2-8NeuAcalpha2-3)Galbeta1-4Glcbeta-Cer(d18:1/16:0); NeuAcalpha2-3Galbeta1-3GalNAcbeta1-4(NeuAcalpha2-8NeuAcalpha2-8NeuAcalpha2-3)Galbeta1-4Glcbeta-Cer(d18:1/18:0); NeuAcalpha2-3Galbeta1-3GalNAcbeta1-4(NeuAcalpha2-8NeuAcalpha2-8NeuAcalpha2-3)Galbeta1-4Glcbeta-Cer(d18:1/20:0); NeuAcalpha2-3Galbeta1-3GalNAcbeta1-4(NeuAcalpha2-8NeuAcalpha2-8NeuAcalpha2-3)Galbeta1-4Glcbeta-Cer(d18:1/22:0); NeuAcalpha2-3Galbeta1-3GalNAcbeta1-4(NeuAcalpha2-8NeuAcalpha2-8NeuAcalpha2-3)Galbeta1-4Glcbeta-Cer(d18:1/24:0); NeuAcalpha2-3Galbeta1-3GalNAcbeta1-4(NeuAcalpha2-8NeuAcalpha2-8NeuAcalpha2-3)Galbeta1-4Glcbeta-Cer(d18:1/24:1(15Z)); NeuAcalpha2-3Galbeta1-3GalNAcbeta1-4(NeuAcalpha2-8NeuAcalpha2-8NeuAcalpha2-3)Galbeta1-4Glcbeta-Cer(d18:1/26:0); NeuAcalpha2-3Galbeta1-3GalNAcbeta1-4(NeuAcalpha2-8NeuAcalpha2-8NeuAcalpha2-3)Galbeta1-4Glcbeta-Cer(d18:1/26:1(17Z)); NeuAcalpha2-3Galbeta1-3GalNAcbeta1-4(NeuAcalpha2-8NeuGcalpha2-3)Galbeta1-4Glcbeta-Cer; NeuAcalpha2-3Galbeta1-3GalNAcbeta1-4(NeuAcalpha2-8NeuGcalpha2-3)Galbeta1-4Glcbeta-Cer(d18:1/16:0); NeuAcalpha2-3Galbeta1-3GalNAcbeta1-4(NeuAcalpha2-8NeuGcalpha2-3)Galbeta1-4Glcbeta-Cer(d18:1/18:0); NeuAcalpha2-3Galbeta1-3GalNAcbeta1-4(NeuAcalpha2-8NeuGcalpha2-3)Galbeta1-4Glcbeta-Cer(d18:1/20:0); NeuAcalpha2-3Galbeta1-3GalNAcbeta1-4(NeuAcalpha2-8NeuGcalpha2-3)Galbeta1-4Glcbeta-Cer(d18:1/22:0); NeuAcalpha2-3Galbeta1-3GalNAcbeta1-4(NeuAcalpha2-8NeuGcalpha2-3)Galbeta1-4Glcbeta-Cer(d18:1/24:0); NeuAcalpha2-3Galbeta1-3GalNAcbeta1-4(NeuAcalpha2-8NeuGcalpha2-3)Galbeta1-4Glcbeta-Cer(d18:1/24:1(15Z)); NeuAcalpha2-3Galbeta1-3GalNAcbeta1-4(NeuAcalpha2-8NeuGcalpha2-3)Galbeta1-4Glcbeta-Cer(d18:1/26:0); NeuAcalpha2-3Galbeta1-3GalNAcbeta1-4(NeuAcalpha2-8NeuGcalpha2-3)Galbeta1-4Glcbeta-Cer(d18:1/26:1(17Z)); NeuAcalpha2-3Galbeta1-3GalNAcbeta1-4(NeuGcalpha2-3)Galbeta1-4Glcbeta-Cer; NeuAcalpha2-3Galbeta1-3GalNAcbeta1-4(NeuGcalpha2-3)Galbeta1-4Glcbeta-Cer(d18:1/16:0); NeuAcalpha2-3Galbeta1-3GalNAcbeta1-4(NeuGcalpha2-3)Galbeta1-4Glcbeta-Cer(d18:1/18:0); NeuAcalpha2-3Galbeta1-3GalNAcbeta1-4(NeuGcalpha2-3)Galbeta1-4Glcbeta-Cer(d18:1/20:0); NeuAcalpha2-3Galbeta1-3GalNAcbeta1-4(NeuGcalpha2-3)Galbeta1-4Glcbeta-Cer(d18:1/22:0); NeuAcalpha2-3Galbeta1-3GalNAcbeta1-4(NeuGcalpha2-3)Galbeta1-4Glcbeta-Cer(d18:1/24:0); NeuAcalpha2-3Galbeta1-3GalNAcbeta1-4(NeuGcalpha2-3)Galbeta1-4Glcbeta-Cer(d18:1/24:1(15Z)); NeuAcalpha2-3Galbeta1-3GalNAcbeta1-4(NeuGcalpha2-3)Galbeta1-4Glcbeta-Cer(d18:1/26:0); NeuAcalpha2-3Galbeta1-3GalNAcbeta1-4(NeuGcalpha2-3)Galbeta1-4Glcbeta-Cer(d18:1/26:1(17Z)); NeuAcalpha2-3Galbeta1-3GalNAcbeta1-4Galbeta1-4Glcbeta-Cer; NeuAcalpha2-3Galbeta1-3GalNAcbeta1-4Galbeta1-4Glcbeta-Cer(d18:1/16:0); NeuAcalpha2-3Galbeta1-3GalNAcbeta1-4Galbeta1-4Glcbeta-Cer(d18:1/18:0); NeuAcalpha2-3Galbeta1-3GalNAcbeta1-4Galbeta1-4Glcbeta-Cer(d18:1/20:0); NeuAcalpha2-3Galbeta1-3GalNAcbeta1-4Galbeta1-4Glcbeta-Cer(d18:1/22:0); NeuAcalpha2-3Galbeta1-3GalNAcbeta1-4Galbeta1-4Glcbeta-Cer(d18:1/24:0); NeuAcalpha2-3Galbeta1-3GalNAcbeta1-4Galbeta1-4Glcbeta-Cer(d18:1/24:1(15Z)); NeuAcalpha2-3Galbeta1-3GalNAcbeta1-4Galbeta1-4Glcbeta-Cer(d18:1/26:0); NeuAcalpha2-3Galbeta1-3GalNAcbeta1-4Galbeta1-4Glcbeta-Cer(d18:1/26:1(17Z)); NeuAcalpha2-3Galbeta1-3GlcNAcbeta1-3Galbeta1-4Glcbeta-Cer; NeuAcalpha2-3Galbeta1-3GlcNAcbeta1-3Galbeta1-4Glcbeta-Cer(d18:1/16:0); NeuAcalpha2-3Galbeta1-3GlcNAcbeta1-3Galbeta1-4Glcbeta-Cer(d18:1/18:0); NeuAcalpha2-3Galbeta1-3GlcNAcbeta1-3Galbeta1-4Glcbeta-Cer(d18:1/20:0); NeuAcalpha2-3Galbeta1-3GlcNAcbeta1-3Galbeta1-4Glcbeta-Cer(d18:1/22:0); NeuAcalpha2-3Galbeta1-3GlcNAcbeta1-3Galbeta1-4Glcbeta-Cer(d18:1/24:0); NeuAcalpha2-3Galbeta1-3GlcNAcbeta1-3Galbeta1-4Glcbeta-Cer(d18:1/24:1(15Z)); NeuAcalpha2-3Galbeta1-3GlcNAcbeta1-3Galbeta1-4Glcbeta-Cer(d18:1/26:0); NeuAcalpha2-3Galbeta1-3GlcNAcbeta1-3Galbeta1-4Glcbeta-Cer(d18:1/26:1(17Z)); NeuAcalpha2-3Galbeta1-3GlcNAcbeta1-3Galbeta1-4GlcNAcbeta1-3Galbeta1-4Glcbeta-Cer; NeuAcalpha2-3Galbeta1-3GlcNAcbeta1-3Galbeta1-4GlcNAcbeta1-3Galbeta1-4Glcbeta-Cer(d18:1/16:0); NeuAcalpha2-3Galbeta1-3GlcNAcbeta1-3Galbeta1-4GlcNAcbeta1-3Galbeta1-4Glcbeta-Cer(d18:1/18:0); NeuAcalpha2-3Galbeta1-3GlcNAcbeta1-3Galbeta1-4GlcNAcbeta1-3Galbeta1-4Glcbeta-Cer(d18:1/20:0); NeuAcalpha2-3Galbeta1-3GlcNAcbeta1-3Galbeta1-4GlcNAcbeta1-3Galbeta1-4Glcbeta-Cer(d18:1/22:0); NeuAcalpha2-3Galbeta1-3GlcNAcbeta1-3Galbeta1-4GlcNAcbeta1-3Galbeta1-4Glcbeta-Cer(d18:1/24:0); NeuAcalpha2-3Galbeta1-3GlcNAcbeta1-3Galbeta1-4GlcNAcbeta1-3Galbeta1-4Glcbeta-Cer(d18:1/24:1(15Z)); NeuAcalpha2-3Galbeta1-3GlcNAcbeta1-3Galbeta1-4GlcNAcbeta1-3Galbeta1-4Glcbeta-Cer(d18:1/26:0); NeuAcalpha2-3Galbeta1-3GlcNAcbeta1-3Galbeta1-4GlcNAcbeta1-3Galbeta1-4Glcbeta-Cer(d18:1/26:1(17Z)); NeuAcalpha2-3Galbeta1-4(Fucalpha1-3)GlcNAcbeta1-3Galalpha1-3(GalNAcbeta1-4)Galbeta1-4Glcbeta-Cer; NeuAcalpha2-3Galbeta1-4(Fucalpha1-3)GlcNAcbeta1-3Galalpha1-3(GalNAcbeta1-4)Galbeta1-4Glcbeta-Cer(d18:1/16:0); NeuAcalpha2-3Galbeta1-4(Fucalpha1-3)GlcNAcbeta1-3Galalpha1-3(GalNAcbeta1-4)Galbeta1-4Glcbeta-Cer(d18:1/18:0); NeuAcalpha2-3Galbeta1-4(Fucalpha1-3)GlcNAcbeta1-3Galalpha1-3(GalNAcbeta1-4)Galbeta1-4Glcbeta-Cer(d18:1/20:0); NeuAcalpha2-3Galbeta1-4(Fucalpha1-3)GlcNAcbeta1-3Galalpha1-3(GalNAcbeta1-4)Galbeta1-4Glcbeta-Cer(d18:1/22:0); NeuAcalpha2-3Galbeta1-4(Fucalpha1-3)GlcNAcbeta1-3Galalpha1-3(GalNAcbeta1-4)Galbeta1-4Glcbeta-Cer(d18:1/24:0); NeuAcalpha2-3Galbeta1-4(Fucalpha1-3)GlcNAcbeta1-3Galalpha1-3(GalNAcbeta1-4)Galbeta1-4Glcbeta-Cer(d18:1/24:1(15Z)); NeuAcalpha2-3Galbeta1-4(Fucalpha1-3)GlcNAcbeta1-3Galalpha1-3(GalNAcbeta1-4)Galbeta1-4Glcbeta-Cer(d18:1/26:0); NeuAcalpha2-3Galbeta1-4(Fucalpha1-3)GlcNAcbeta1-3Galalpha1-3(GalNAcbeta1-4)Galbeta1-4Glcbeta-Cer(d18:1/26:1(17Z)); NeuAcalpha2-3Galbeta1-4(Fucalpha1-3)GlcNAcbeta1-3Galalpha1-3Galbeta1-4Glcbeta-Cer; NeuAcalpha2-3Galbeta1-4(Fucalpha1-3)GlcNAcbeta1-3Galalpha1-3Galbeta1-4Glcbeta-Cer; NeuAcalpha2-3Galbeta1-4(Fucalpha1-3)GlcNAcbeta1-3Galalpha1-3Galbeta1-4Glcbeta-Cer(d18:1/16:0); NeuAcalpha2-3Galbeta1-4(Fucalpha1-3)GlcNAcbeta1-3Galalpha1-3Galbeta1-4Glcbeta-Cer(d18:1/18:0); NeuAcalpha2-3Galbeta1-4(Fucalpha1-3)GlcNAcbeta1-3Galalpha1-3Galbeta1-4Glcbeta-Cer(d18:1/20:0); NeuAcalpha2-3Galbeta1-4(Fucalpha1-3)GlcNAcbeta1-3Galalpha1-3Galbeta1-4Glcbeta-Cer(d18:1/22:0); NeuAcalpha2-3Galbeta1-4(Fucalpha1-3)GlcNAcbeta1-3Galalpha1-3Galbeta1-4Glcbeta-Cer(d18:1/24:0); NeuAcalpha2-3Galbeta1-4(Fucalpha1-3)GlcNAcbeta1-3Galalpha1-3Galbeta1-4Glcbeta-Cer(d18:1/24:1(15Z)); NeuAcalpha2-3Galbeta1-4(Fucalpha1-3)GlcNAcbeta1-3Galalpha1-3Galbeta1-4Glcbeta-Cer(d18:1/26:0); NeuAcalpha2-3Galbeta1-4(Fucalpha1-3)GlcNAcbeta1-3Galalpha1-3Galbeta1-4Glcbeta-Cer(d18:1/26:1(17Z)); NeuAcalpha2-3Galbeta1-4(Fucalpha1-3)GlcNAcbeta1-3Galbeta1-4(Fucalpha1-3)GlcNAcbeta1-3Galbeta1-4(Fucalpha1-3)GlcNAcbeta1-3Galbeta1-4Glcbeta-Cer; NeuAcalpha2-3Galbeta1-4(Fucalpha1-3)GlcNAcbeta1-3Galbeta1-4(Fucalpha1-3)GlcNAcbeta1-3Galbeta1-4(Fucalpha1-3)GlcNAcbeta1-3Galbeta1-4Glcbeta-Cer(d18:1/16:0); NeuAcalpha2-3Galbeta1-4(Fucalpha1-3)GlcNAcbeta1-3Galbeta1-4(Fucalpha1-3)GlcNAcbeta1-3Galbeta1-4(Fucalpha1-3)GlcNAcbeta1-3Galbeta1-4Glcbeta-Cer(d18:1/18:0); NeuAcalpha2-3Galbeta1-4(Fucalpha1-3)GlcNAcbeta1-3Galbeta1-4(Fucalpha1-3)GlcNAcbeta1-3Galbeta1-4(Fucalpha1-3)GlcNAcbeta1-3Galbeta1-4Glcbeta-Cer(d18:1/20:0); NeuAcalpha2-3Galbeta1-4(Fucalpha1-3)GlcNAcbeta1-3Galbeta1-4(Fucalpha1-3)GlcNAcbeta1-3Galbeta1-4(Fucalpha1-3)GlcNAcbeta1-3Galbeta1-4Glcbeta-Cer(d18:1/22:0); NeuAcalpha2-3Galbeta1-4(Fucalpha1-3)GlcNAcbeta1-3Galbeta1-4(Fucalpha1-3)GlcNAcbeta1-3Galbeta1-4(Fucalpha1-3)GlcNAcbeta1-3Galbeta1-4Glcbeta-Cer(d18:1/24:0); NeuAcalpha2-3Galbeta1-4(Fucalpha1-3)GlcNAcbeta1-3Galbeta1-4(Fucalpha1-3)GlcNAcbeta1-3Galbeta1-4(Fucalpha1-3)GlcNAcbeta1-3Galbeta1-4Glcbeta-Cer(d18:1/24:1(15Z)); NeuAcalpha2-3Galbeta1-4(Fucalpha1-3)GlcNAcbeta1-3Galbeta1-4(Fucalpha1-3)GlcNAcbeta1-3Galbeta1-4(Fucalpha1-3)GlcNAcbeta1-3Galbeta1-4Glcbeta-Cer(d18:1/26:0); NeuAcalpha2-3Galbeta1-4(Fucalpha1-3)GlcNAcbeta1-3Galbeta1-4(Fucalpha1-3)GlcNAcbeta1-3Galbeta1-4(Fucalpha1-3)GlcNAcbeta1-3Galbeta1-4Glcbeta-Cer(d18:1/26:1(17Z)); NeuAcalpha2-3Galbeta1-4(Fucalpha1-3)GlcNAcbeta1-3Galbeta1-4(Fucalpha1-3)GlcNAcbeta1-3Galbeta1-4Glcbeta-Cer; NeuAcalpha2-3Galbeta1-4(Fucalpha1-3)GlcNAcbeta1-3Galbeta1-4(Fucalpha1-3)GlcNAcbeta1-3Galbeta1-4Glcbeta-Cer(d18:1/16:0); NeuAcalpha2-3Galbeta1-4(Fucalpha1-3)GlcNAcbeta1-3Galbeta1-4(Fucalpha1-3)GlcNAcbeta1-3Galbeta1-4Glcbeta-Cer(d18:1/18:0); NeuAcalpha2-3Galbeta1-4(Fucalpha1-3)GlcNAcbeta1-3Galbeta1-4(Fucalpha1-3)GlcNAcbeta1-3Galbeta1-4Glcbeta-Cer(d18:1/20:0); NeuAcalpha2-3Galbeta1-4(Fucalpha1-3)GlcNAcbeta1-3Galbeta1-4(Fucalpha1-3)GlcNAcbeta1-3Galbeta1-4Glcbeta-Cer(d18:1/22:0); NeuAcalpha2-3Galbeta1-4(Fucalpha1-3)GlcNAcbeta1-3Galbeta1-4(Fucalpha1-3)GlcNAcbeta1-3Galbeta1-4Glcbeta-Cer(d18:1/24:0); NeuAcalpha2-3Galbeta1-4(Fucalpha1-3)GlcNAcbeta1-3Galbeta1-4(Fucalpha1-3)GlcNAcbeta1-3Galbeta1-4Glcbeta-Cer(d18:1/24:1(15Z)); NeuAcalpha2-3Galbeta1-4(Fucalpha1-3)GlcNAcbeta1-3Galbeta1-4(Fucalpha1-3)GlcNAcbeta1-3Galbeta1-4Glcbeta-Cer(d18:1/26:0); NeuAcalpha2-3Galbeta1-4(Fucalpha1-3)GlcNAcbeta1-3Galbeta1-4(Fucalpha1-3)GlcNAcbeta1-3Galbeta1-4Glcbeta-Cer(d18:1/26:1(17Z)); NeuAcalpha2-3Galbeta1-4(Fucalpha1-3)GlcNAcbeta1-3Galbeta1-4(Fucalpha1-3)GlcNAcbeta1-3Galbeta1-4GlcNAcbeta1-3Galbeta1-4Glcbeta-Cer; NeuAcalpha2-3Galbeta1-4(Fucalpha1-3)GlcNAcbeta1-3Galbeta1-4(Fucalpha1-3)GlcNAcbeta1-3Galbeta1-4GlcNAcbeta1-3Galbeta1-4Glcbeta-Cer(d18:1/16:0); NeuAcalpha2-3Galbeta1-4(Fucalpha1-3)GlcNAcbeta1-3Galbeta1-4(Fucalpha1-3)GlcNAcbeta1-3Galbeta1-4GlcNAcbeta1-3Galbeta1-4Glcbeta-Cer(d18:1/18:0); NeuAcalpha2-3Galbeta1-4(Fucalpha1-3)GlcNAcbeta1-3Galbeta1-4(Fucalpha1-3)GlcNAcbeta1-3Galbeta1-4GlcNAcbeta1-3Galbeta1-4Glcbeta-Cer(d18:1/20:0); NeuAcalpha2-3Galbeta1-4(Fucalpha1-3)GlcNAcbeta1-3Galbeta1-4(Fucalpha1-3)GlcNAcbeta1-3Galbeta1-4GlcNAcbeta1-3Galbeta1-4Glcbeta-Cer(d18:1/22:0); NeuAcalpha2-3Galbeta1-4(Fucalpha1-3)GlcNAcbeta1-3Galbeta1-4(Fucalpha1-3)GlcNAcbeta1-3Galbeta1-4GlcNAcbeta1-3Galbeta1-4Glcbeta-Cer(d18:1/24:0); NeuAcalpha2-3Galbeta1-4(Fucalpha1-3)GlcNAcbeta1-3Galbeta1-4(Fucalpha1-3)GlcNAcbeta1-3Galbeta1-4GlcNAcbeta1-3Galbeta1-4Glcbeta-Cer(d18:1/24:1(15Z)); NeuAcalpha2-3Galbeta1-4(Fucalpha1-3)GlcNAcbeta1-3Galbeta1-4(Fucalpha1-3)GlcNAcbeta1-3Galbeta1-4GlcNAcbeta1-3Galbeta1-4Glcbeta-Cer(d18:1/26:0); NeuAcalpha2-3Galbeta1-4(Fucalpha1-3)GlcNAcbeta1-3Galbeta1-4(Fucalpha1-3)GlcNAcbeta1-3Galbeta1-4GlcNAcbeta1-3Galbeta1-4Glcbeta-Cer(d18:1/26:1(17Z)); NeuAcalpha2-3Galbeta1-4(Fucalpha1-3)GlcNAcbeta1-3Galbeta1-4Glcbeta-Cer; NeuAcalpha2-3Galbeta1-4(Fucalpha1-3)GlcNAcbeta1-3Galbeta1-4Glcbeta-Cer(d18:1/16:0); NeuAcalpha2-3Galbeta1-4(Fucalpha1-3)GlcNAcbeta1-3Galbeta1-4Glcbeta-Cer(d18:1/18:0); NeuAcalpha2-3Galbeta1-4(Fucalpha1-3)GlcNAcbeta1-3Galbeta1-4Glcbeta-Cer(d18:1/20:0); NeuAcalpha2-3Galbeta1-4(Fucalpha1-3)GlcNAcbeta1-3Galbeta1-4Glcbeta-Cer(d18:1/22:0); NeuAcalpha2-3Galbeta1-4(Fucalpha1-3)GlcNAcbeta1-3Galbeta1-4Glcbeta-Cer(d18:1/24:0); NeuAcalpha2-3Galbeta1-4(Fucalpha1-3)GlcNAcbeta1-3Galbeta1-4Glcbeta-Cer(d18:1/24:1(15Z)); NeuAcalpha2-3Galbeta1-4(Fucalpha1-3)GlcNAcbeta1-3Galbeta1-4Glcbeta-Cer(d18:1/26:0); NeuAcalpha2-3Galbeta1-4(Fucalpha1-3)GlcNAcbeta1-3Galbeta1-4Glcbeta-Cer(d18:1/26:1(17Z)); NeuAcalpha2-3Galbeta1-4(Fucalpha1-3)GlcNAcbeta1-3Galbeta1-4GlcNAcbeta1-3Galbeta1-4(Fucalpha1-3)GlcNAcbeta1-3Galbeta1-4Glcbeta-Cer; NeuAcalpha2-3Galbeta1-4(Fucalpha1-3)GlcNAcbeta1-3Galbeta1-4GlcNAcbeta1-3Galbeta1-4(Fucalpha1-3)GlcNAcbeta1-3Galbeta1-4Glcbeta-Cer(d18:1/16:0); NeuAcalpha2-3Galbeta1-4(Fucalpha1-3)GlcNAcbeta1-3Galbeta1-4GlcNAcbeta1-3Galbeta1-4(Fucalpha1-3)GlcNAcbeta1-3Galbeta1-4Glcbeta-Cer(d18:1/18:0); NeuAcalpha2-3Galbeta1-4(Fucalpha1-3)GlcNAcbeta1-3Galbeta1-4GlcNAcbeta1-3Galbeta1-4(Fucalpha1-3)GlcNAcbeta1-3Galbeta1-4Glcbeta-Cer(d18:1/20:0); NeuAcalpha2-3Galbeta1-4(Fucalpha1-3)GlcNAcbeta1-3Galbeta1-4GlcNAcbeta1-3Galbeta1-4(Fucalpha1-3)GlcNAcbeta1-3Galbeta1-4Glcbeta-Cer(d18:1/22:0); NeuAcalpha2-3Galbeta1-4(Fucalpha1-3)GlcNAcbeta1-3Galbeta1-4GlcNAcbeta1-3Galbeta1-4(Fucalpha1-3)GlcNAcbeta1-3Galbeta1-4Glcbeta-Cer(d18:1/24:0); NeuAcalpha2-3Galbeta1-4(Fucalpha1-3)GlcNAcbeta1-3Galbeta1-4GlcNAcbeta1-3Galbeta1-4(Fucalpha1-3)GlcNAcbeta1-3Galbeta1-4Glcbeta-Cer(d18:1/24:1(15Z)); NeuAcalpha2-3Galbeta1-4(Fucalpha1-3)GlcNAcbeta1-3Galbeta1-4GlcNAcbeta1-3Galbeta1-4(Fucalpha1-3)GlcNAcbeta1-3Galbeta1-4Glcbeta-Cer(d18:1/26:0); NeuAcalpha2-3Galbeta1-4(Fucalpha1-3)GlcNAcbeta1-3Galbeta1-4GlcNAcbeta1-3Galbeta1-4(Fucalpha1-3)GlcNAcbeta1-3Galbeta1-4Glcbeta-Cer(d18:1/26:1(17Z)); NeuAcalpha2-3Galbeta1-4(Fucalpha1-3)GlcNAcbeta1-3Galbeta1-4GlcNAcbeta1-3Galbeta1-4Glcbeta-Cer; NeuAcalpha2-3Galbeta1-4(Fucalpha1-3)GlcNAcbeta1-3Galbeta1-4GlcNAcbeta1-3Galbeta1-4Glcbeta-Cer(d18:1/16:0); NeuAcalpha2-3Galbeta1-4(Fucalpha1-3)GlcNAcbeta1-3Galbeta1-4GlcNAcbeta1-3Galbeta1-4Glcbeta-Cer(d18:1/18:0); NeuAcalpha2-3Galbeta1-4(Fucalpha1-3)GlcNAcbeta1-3Galbeta1-4GlcNAcbeta1-3Galbeta1-4Glcbeta-Cer(d18:1/20:0); NeuAcalpha2-3Galbeta1-4(Fucalpha1-3)GlcNAcbeta1-3Galbeta1-4GlcNAcbeta1-3Galbeta1-4Glcbeta-Cer(d18:1/22:0); NeuAcalpha2-3Galbeta1-4(Fucalpha1-3)GlcNAcbeta1-3Galbeta1-4GlcNAcbeta1-3Galbeta1-4Glcbeta-Cer(d18:1/24:0); NeuAcalpha2-3Galbeta1-4(Fucalpha1-3)GlcNAcbeta1-3Galbeta1-4GlcNAcbeta1-3Galbeta1-4Glcbeta-Cer(d18:1/24:1(15Z)); NeuAcalpha2-3Galbeta1-4(Fucalpha1-3)GlcNAcbeta1-3Galbeta1-4GlcNAcbeta1-3Galbeta1-4Glcbeta-Cer(d18:1/26:0); NeuAcalpha2-3Galbeta1-4(Fucalpha1-3)GlcNAcbeta1-3Galbeta1-4GlcNAcbeta1-3Galbeta1-4Glcbeta-Cer(d18:1/26:1(17Z)); NeuAcalpha2-3Galbeta1-4Glcbeta-Cer; NeuAcalpha2-3Galbeta1-4Glcbeta-Cer(d18:1/16:0); NeuAcalpha2-3Galbeta1-4Glcbeta-Cer(d18:1/18:0); NeuAcalpha2-3Galbeta1-4Glcbeta-Cer(d18:1/20:0); NeuAcalpha2-3Galbeta1-4Glcbeta-Cer(d18:1/22:0); NeuAcalpha2-3Galbeta1-4Glcbeta-Cer(d18:1/24:0); NeuAcalpha2-3Galbeta1-4Glcbeta-Cer(d18:1/24:1(15Z)); NeuAcalpha2-3Galbeta1-4Glcbeta-Cer(d18:1/26:0); NeuAcalpha2-3Galbeta1-4Glcbeta-Cer(d18:1/26:1(17Z)); NeuAcalpha2-3Galbeta1-4GlcNAcalpha1-3(Fucalpha1-2Galbeta1-4GlcNAcbeta1-6)Galbeta1-4GlcNAcbeta1-3Galbeta1-4Glcbeta-Cer; NeuAcalpha2-3Galbeta1-4GlcNAcalpha1-3(Fucalpha1-2Galbeta1-4GlcNAcbeta1-6)Galbeta1-4GlcNAcbeta1-3Galbeta1-4Glcbeta-Cer(d18:1/16:0); NeuAcalpha2-3Galbeta1-4GlcNAcalpha1-3(Fucalpha1-2Galbeta1-4GlcNAcbeta1-6)Galbeta1-4GlcNAcbeta1-3Galbeta1-4Glcbeta-Cer(d18:1/18:0); NeuAcalpha2-3Galbeta1-4GlcNAcalpha1-3(Fucalpha1-2Galbeta1-4GlcNAcbeta1-6)Galbeta1-4GlcNAcbeta1-3Galbeta1-4Glcbeta-Cer(d18:1/20:0); NeuAcalpha2-3Galbeta1-4GlcNAcalpha1-3(Fucalpha1-2Galbeta1-4GlcNAcbeta1-6)Galbeta1-4GlcNAcbeta1-3Galbeta1-4Glcbeta-Cer(d18:1/22:0); NeuAcalpha2-3Galbeta1-4GlcNAcalpha1-3(Fucalpha1-2Galbeta1-4GlcNAcbeta1-6)Galbeta1-4GlcNAcbeta1-3Galbeta1-4Glcbeta-Cer(d18:1/24:0); NeuAcalpha2-3Galbeta1-4GlcNAcalpha1-3(Fucalpha1-2Galbeta1-4GlcNAcbeta1-6)Galbeta1-4GlcNAcbeta1-3Galbeta1-4Glcbeta-Cer(d18:1/24:1(15Z)); NeuAcalpha2-3Galbeta1-4GlcNAcalpha1-3(Fucalpha1-2Galbeta1-4GlcNAcbeta1-6)Galbeta1-4GlcNAcbeta1-3Galbeta1-4Glcbeta-Cer(d18:1/26:0); NeuAcalpha2-3Galbeta1-4GlcNAcalpha1-3(Fucalpha1-2Galbeta1-4GlcNAcbeta1-6)Galbeta1-4GlcNAcbeta1-3Galbeta1-4Glcbeta-Cer(d18:1/26:1(17Z)); NeuAcalpha2-3Galbeta1-4GlcNAcalpha1-3(Galbeta1-4GlcNAcbeta1-6)Galbeta1-4GlcNAcbeta1-3Galbeta1-4Glcbeta-Cer; NeuAcalpha2-3Galbeta1-4GlcNAcalpha1-3(Galbeta1-4GlcNAcbeta1-6)Galbeta1-4GlcNAcbeta1-3Galbeta1-4Glcbeta-Cer(d18:1/16:0); NeuAcalpha2-3Galbeta1-4GlcNAcalpha1-3(Galbeta1-4GlcNAcbeta1-6)Galbeta1-4GlcNAcbeta1-3Galbeta1-4Glcbeta-Cer(d18:1/18:0); NeuAcalpha2-3Galbeta1-4GlcNAcalpha1-3(Galbeta1-4GlcNAcbeta1-6)Galbeta1-4GlcNAcbeta1-3Galbeta1-4Glcbeta-Cer(d18:1/20:0); NeuAcalpha2-3Galbeta1-4GlcNAcalpha1-3(Galbeta1-4GlcNAcbeta1-6)Galbeta1-4GlcNAcbeta1-3Galbeta1-4Glcbeta-Cer(d18:1/22:0); NeuAcalpha2-3Galbeta1-4GlcNAcalpha1-3(Galbeta1-4GlcNAcbeta1-6)Galbeta1-4GlcNAcbeta1-3Galbeta1-4Glcbeta-Cer(d18:1/24:0); NeuAcalpha2-3Galbeta1-4GlcNAcalpha1-3(Galbeta1-4GlcNAcbeta1-6)Galbeta1-4GlcNAcbeta1-3Galbeta1-4Glcbeta-Cer(d18:1/24:1(15Z)); NeuAcalpha2-3Galbeta1-4GlcNAcalpha1-3(Galbeta1-4GlcNAcbeta1-6)Galbeta1-4GlcNAcbeta1-3Galbeta1-4Glcbeta-Cer(d18:1/26:0); NeuAcalpha2-3Galbeta1-4GlcNAcalpha1-3(Galbeta1-4GlcNAcbeta1-6)Galbeta1-4GlcNAcbeta1-3Galbeta1-4Glcbeta-Cer(d18:1/26:1(17Z)); NeuAcalpha2-3Galbeta1-4GlcNAcalpha1-3(GlcNAcbeta1-6)Galbeta1-4GlcNAcbeta1-3Galbeta1-4Glcbeta-Cer; NeuAcalpha2-3Galbeta1-4GlcNAcalpha1-3(GlcNAcbeta1-6)Galbeta1-4GlcNAcbeta1-3Galbeta1-4Glcbeta-Cer(d18:1/16:0); NeuAcalpha2-3Galbeta1-4GlcNAcalpha1-3(GlcNAcbeta1-6)Galbeta1-4GlcNAcbeta1-3Galbeta1-4Glcbeta-Cer(d18:1/18:0); NeuAcalpha2-3Galbeta1-4GlcNAcalpha1-3(GlcNAcbeta1-6)Galbeta1-4GlcNAcbeta1-3Galbeta1-4Glcbeta-Cer(d18:1/20:0); NeuAcalpha2-3Galbeta1-4GlcNAcalpha1-3(GlcNAcbeta1-6)Galbeta1-4GlcNAcbeta1-3Galbeta1-4Glcbeta-Cer(d18:1/22:0); NeuAcalpha2-3Galbeta1-4GlcNAcalpha1-3(GlcNAcbeta1-6)Galbeta1-4GlcNAcbeta1-3Galbeta1-4Glcbeta-Cer(d18:1/24:0); NeuAcalpha2-3Galbeta1-4GlcNAcalpha1-3(GlcNAcbeta1-6)Galbeta1-4GlcNAcbeta1-3Galbeta1-4Glcbeta-Cer(d18:1/24:1(15Z)); NeuAcalpha2-3Galbeta1-4GlcNAcalpha1-3(GlcNAcbeta1-6)Galbeta1-4GlcNAcbeta1-3Galbeta1-4Glcbeta-Cer(d18:1/26:0); NeuAcalpha2-3Galbeta1-4GlcNAcalpha1-3(GlcNAcbeta1-6)Galbeta1-4GlcNAcbeta1-3Galbeta1-4Glcbeta-Cer(d18:1/26:1(17Z)); NeuAcalpha2-3Galbeta1-4GlcNAcalpha1-3(NeuAcalpha2-3Galbeta1-4GlcNAcbeta1-6)Galbeta1-4GlcNAcbeta1-3Galbeta1-4Glcbeta-Cer; NeuAcalpha2-3Galbeta1-4GlcNAcalpha1-3(NeuAcalpha2-3Galbeta1-4GlcNAcbeta1-6)Galbeta1-4GlcNAcbeta1-3Galbeta1-4Glcbeta-Cer(d18:1/16:0); NeuAcalpha2-3Galbeta1-4GlcNAcalpha1-3(NeuAcalpha2-3Galbeta1-4GlcNAcbeta1-6)Galbeta1-4GlcNAcbeta1-3Galbeta1-4Glcbeta-Cer(d18:1/18:0); NeuAcalpha2-3Galbeta1-4GlcNAcalpha1-3(NeuAcalpha2-3Galbeta1-4GlcNAcbeta1-6)Galbeta1-4GlcNAcbeta1-3Galbeta1-4Glcbeta-Cer(d18:1/20:0); NeuAcalpha2-3Galbeta1-4GlcNAcalpha1-3(NeuAcalpha2-3Galbeta1-4GlcNAcbeta1-6)Galbeta1-4GlcNAcbeta1-3Galbeta1-4Glcbeta-Cer(d18:1/22:0); NeuAcalpha2-3Galbeta1-4GlcNAcalpha1-3(NeuAcalpha2-3Galbeta1-4GlcNAcbeta1-6)Galbeta1-4GlcNAcbeta1-3Galbeta1-4Glcbeta-Cer(d18:1/24:0); NeuAcalpha2-3Galbeta1-4GlcNAcalpha1-3(NeuAcalpha2-3Galbeta1-4GlcNAcbeta1-6)Galbeta1-4GlcNAcbeta1-3Galbeta1-4Glcbeta-Cer(d18:1/24:1(15Z)); NeuAcalpha2-3Galbeta1-4GlcNAcalpha1-3(NeuAcalpha2-3Galbeta1-4GlcNAcbeta1-6)Galbeta1-4GlcNAcbeta1-3Galbeta1-4Glcbeta-Cer(d18:1/26:0); NeuAcalpha2-3Galbeta1-4GlcNAcalpha1-3(NeuAcalpha2-3Galbeta1-4GlcNAcbeta1-6)Galbeta1-4GlcNAcbeta1-3Galbeta1-4Glcbeta-Cer(d18:1/26:1(17Z)); NeuAcalpha2-3Galbeta1-4GlcNAcalpha1-3Galbeta1-4GlcNAcbeta1-3Galbeta1-4Glcbeta-Cer; NeuAcalpha2-3Galbeta1-4GlcNAcalpha1-3Galbeta1-4GlcNAcbeta1-3Galbeta1-4Glcbeta-Cer(d18:1/16:0); NeuAcalpha2-3Galbeta1-4GlcNAcalpha1-3Galbeta1-4GlcNAcbeta1-3Galbeta1-4Glcbeta-Cer(d18:1/18:0); NeuAcalpha2-3Galbeta1-4GlcNAcalpha1-3Galbeta1-4GlcNAcbeta1-3Galbeta1-4Glcbeta-Cer(d18:1/20:0); NeuAcalpha2-3Galbeta1-4GlcNAcalpha1-3Galbeta1-4GlcNAcbeta1-3Galbeta1-4Glcbeta-Cer(d18:1/22:0); NeuAcalpha2-3Galbeta1-4GlcNAcalpha1-3Galbeta1-4GlcNAcbeta1-3Galbeta1-4Glcbeta-Cer(d18:1/24:0); NeuAcalpha2-3Galbeta1-4GlcNAcalpha1-3Galbeta1-4GlcNAcbeta1-3Galbeta1-4Glcbeta-Cer(d18:1/24:1(15Z)); NeuAcalpha2-3Galbeta1-4GlcNAcalpha1-3Galbeta1-4GlcNAcbeta1-3Galbeta1-4Glcbeta-Cer(d18:1/26:0); NeuAcalpha2-3Galbeta1-4GlcNAcalpha1-3Galbeta1-4GlcNAcbeta1-3Galbeta1-4Glcbeta-Cer(d18:1/26:1(17Z)); NeuAcalpha2-3Galbeta1-4GlcNAcbeta1-3(Fucalpha1-2Galbeta1-4GlcNAcbeta1-6)Galbeta1-4GlcNAcbeta1-3Galbeta1-4Glcbeta-Cer; NeuAcalpha2-3Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/16:0); NeuAcalpha2-3Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/18:0); NeuAcalpha2-3Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/20:0); NeuAcalpha2-3Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/22:0); NeuAcalpha2-3Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/24:0); NeuAcalpha2-3Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/24:1(15Z)); NeuAcalpha2-3Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/26:0); NeuAcalpha2-3Galbeta1-4GlcNAcbeta1-3(Fucalpha1-2Galbeta1-4GlcNAcbeta1-6)Galbeta1-4GlcNAcbeta1-3Galbeta1-4Glcbeta-Cer(d18:1/26:1(17Z)); NeuAcalpha2-3Galbeta1-4GlcNAcbeta1-3(Fucx1-xGalbeta1-4GlcNAcbeta1-6)Galbeta1-4GlcNAcbeta1-3Galbeta1-4Glcbeta-Cer; NeuAcalpha2-3Galbeta1-4GlcNAcbeta1-3(Galalpha1-3(Fucalpha1-2)Galbeta1-4GlcNAcbeta1-6)Galbeta1-4GlcNAcbeta1-3Galbeta1-4Glcbeta-Cer; NeuAcalpha2-3Galbeta1-4GlcNAcbeta1-3(Galalpha1-3(Fucalpha1-2)Galbeta1-4GlcNAcbeta1-6)Galbeta1-4GlcNAcbeta1-3Galbeta1-4Glcbeta-Cer(d18:1/16:0); NeuAcalpha2-3Galbeta1-4GlcNAcbeta1-3(Galalpha1-3(Fucalpha1-2)Galbeta1-4GlcNAcbeta1-6)Galbeta1-4GlcNAcbeta1-3Galbeta1-4Glcbeta-Cer(d18:1/18:0); NeuAcalpha2-3Galbeta1-4GlcNAcbeta1-3(Galalpha1-3(Fucalpha1-2)Galbeta1-4GlcNAcbeta1-6)Galbeta1-4GlcNAcbeta1-3Galbeta1-4Glcbeta-Cer(d18:1/20:0); NeuAcalpha2-3Galbeta1-4GlcNAcbeta1-3(Galalpha1-3(Fucalpha1-2)Galbeta1-4GlcNAcbeta1-6)Galbeta1-4GlcNAcbeta1-3Galbeta1-4Glcbeta-Cer(d18:1/22:0); NeuAcalpha2-3Galbeta1-4GlcNAcbeta1-3(Galalpha1-3(Fucalpha1-2)Galbeta1-4GlcNAcbeta1-6)Galbeta1-4GlcNAcbeta1-3Galbeta1-4Glcbeta-Cer(d18:1/24:0); NeuAcalpha2-3Galbeta1-4GlcNAcbeta1-3(Galalpha1-3(Fucalpha1-2)Galbeta1-4GlcNAcbeta1-6)Galbeta1-4GlcNAcbeta1-3Galbeta1-4Glcbeta-Cer(d18:1/24:1(15Z)); NeuAcalpha2-3Galbeta1-4GlcNAcbeta1-3(Galalpha1-3(Fucalpha1-2)Galbeta1-4GlcNAcbeta1-6)Galbeta1-4GlcNAcbeta1-3Galbeta1-4Glcbeta-Cer(d18:1/26:0); NeuAcalpha2-3Galbeta1-4GlcNAcbeta1-3(Galalpha1-3(Fucalpha1-2)Galbeta1-4GlcNAcbeta1-6)Galbeta1-4GlcNAcbeta1-3Galbeta1-4Glcbeta-Cer(d18:1/26:1(17Z)); NeuAcalpha2-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer; NeuAcalpha2-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/16:0); NeuAcalpha2-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/18:0); NeuAcalpha2-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/20:0); NeuAcalpha2-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/22:0); NeuAcalpha2-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:0); NeuAcalpha2-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:1(15Z)); NeuAcalpha2-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:0); NeuAcalpha2-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:1(17Z)); NeuAcalpha2-3Galbeta1-4GlcNAcbeta1-3(Galbeta1-4GlcNAcbeta1-6)Galbeta1-4GlcNAcbeta1-3Galbeta1-4Glcbeta-Cer; NeuAcalpha2-3Galbeta1-4GlcNAcbeta1-3(Galbeta1-4GlcNAcbeta1-6)Galbeta1-4GlcNAcbeta1-3Galbeta1-4Glcbeta-Cer(d18:1/16:0); NeuAcalpha2-3Galbeta1-4GlcNAcbeta1-3(Galbeta1-4GlcNAcbeta1-6)Galbeta1-4GlcNAcbeta1-3Galbeta1-4Glcbeta-Cer(d18:1/18:0); NeuAcalpha2-3Galbeta1-4GlcNAcbeta1-3(Galbeta1-4GlcNAcbeta1-6)Galbeta1-4GlcNAcbeta1-3Galbeta1-4Glcbeta-Cer(d18:1/20:0); NeuAcalpha2-3Galbeta1-4GlcNAcbeta1-3(Galbeta1-4GlcNAcbeta1-6)Galbeta1-4GlcNAcbeta1-3Galbeta1-4Glcbeta-Cer(d18:1/22:0); NeuAcalpha2-3Galbeta1-4GlcNAcbeta1-3(Galbeta1-4GlcNAcbeta1-6)Galbeta1-4GlcNAcbeta1-3Galbeta1-4Glcbeta-Cer(d18:1/24:0); NeuAcalpha2-3Galbeta1-4GlcNAcbeta1-3(Galbeta1-4GlcNAcbeta1-6)Galbeta1-4GlcNAcbeta1-3Galbeta1-4Glcbeta-Cer(d18:1/24:1(15Z)); NeuAcalpha2-3Galbeta1-4GlcNAcbeta1-3(Galbeta1-4GlcNAcbeta1-6)Galbeta1-4GlcNAcbeta1-3Galbeta1-4Glcbeta-Cer(d18:1/26:0); NeuAcalpha2-3Galbeta1-4GlcNAcbeta1-3(Galbeta1-4GlcNAcbeta1-6)Galbeta1-4GlcNAcbeta1-3Galbeta1-4Glcbeta-Cer(d18:1/26:1(17Z)); NeuAcalpha2-3Galbeta1-4GlcNAcbeta1-3(GalNAcbeta1-4)Galbeta1-4Glcbeta-Cer; NeuAcalpha2-3Galbeta1-4GlcNAcbeta1-3(GalNAcbeta1-4)Galbeta1-4Glcbeta-Cer(d18:1/16:0); NeuAcalpha2-3Galbeta1-4GlcNAcbeta1-3(GalNAcbeta1-4)Galbeta1-4Glcbeta-Cer(d18:1/18:0); NeuAcalpha2-3Galbeta1-4GlcNAcbeta1-3(GalNAcbeta1-4)Galbeta1-4Glcbeta-Cer(d18:1/20:0); NeuAcalpha2-3Galbeta1-4GlcNAcbeta1-3(GalNAcbeta1-4)Galbeta1-4Glcbeta-Cer(d18:1/22:0); NeuAcalpha2-3Galbeta1-4GlcNAcbeta1-3(GalNAcbeta1-4)Galbeta1-4Glcbeta-Cer(d18:1/24:0); NeuAcalpha2-3Galbeta1-4GlcNAcbeta1-3(GalNAcbeta1-4)Galbeta1-4Glcbeta-Cer(d18:1/24:1(15Z)); NeuAcalpha2-3Galbeta1-4GlcNAcbeta1-3(GalNAcbeta1-4)Galbeta1-4Glcbeta-Cer(d18:1/26:0); NeuAcalpha2-3Galbeta1-4GlcNAcbeta1-3(GalNAcbeta1-4)Galbeta1-4Glcbeta-Cer(d18:1/26:1(17Z)); NeuAcalpha2-3Galbeta1-4GlcNAcbeta1-3(Galx1-3(Fucalpha1-2)Galbeta1-4GlcNAcbeta1-6}Galbeta1-4GlcNAcbeta1-3Galbeta1-4Glcbeta-Cer; NeuAcalpha2-3Galbeta1-4GlcNAcbeta1-3(Galx1-3(Fucalpha1-2)Galbeta1-4GlcNAcbeta1-6}Galbeta1-4GlcNAcbeta1-3Galbeta1-4Glcbeta-Cer(d18:1/16:0); NeuAcalpha2-3Galbeta1-4GlcNAcbeta1-3(Galx1-3(Fucalpha1-2)Galbeta1-4GlcNAcbeta1-6}Galbeta1-4GlcNAcbeta1-3Galbeta1-4Glcbeta-Cer(d18:1/18:0); NeuAcalpha2-3Galbeta1-4GlcNAcbeta1-3(Galx1-3(Fucalpha1-2)Galbeta1-4GlcNAcbeta1-6}Galbeta1-4GlcNAcbeta1-3Galbeta1-4Glcbeta-Cer(d18:1/20:0); NeuAcalpha2-3Galbeta1-4GlcNAcbeta1-3(Galx1-3(Fucalpha1-2)Galbeta1-4GlcNAcbeta1-6}Galbeta1-4GlcNAcbeta1-3Galbeta1-4Glcbeta-Cer(d18:1/22:0); NeuAcalpha2-3Galbeta1-4GlcNAcbeta1-3(Galx1-3(Fucalpha1-2)Galbeta1-4GlcNAcbeta1-6}Galbeta1-4GlcNAcbeta1-3Galbeta1-4Glcbeta-Cer(d18:1/24:0); NeuAcalpha2-3Galbeta1-4GlcNAcbeta1-3(Galx1-3(Fucalpha1-2)Galbeta1-4GlcNAcbeta1-6}Galbeta1-4GlcNAcbeta1-3Galbeta1-4Glcbeta-Cer(d18:1/24:1(15Z)); NeuAcalpha2-3Galbeta1-4GlcNAcbeta1-3(Galx1-3(Fucalpha1-2)Galbeta1-4GlcNAcbeta1-6}Galbeta1-4GlcNAcbeta1-3Galbeta1-4Glcbeta-Cer(d18:1/26:0); NeuAcalpha2-3Galbeta1-4GlcNAcbeta1-3(Galx1-3(Fucalpha1-2)Galbeta1-4GlcNAcbeta1-6}Galbeta1-4GlcNAcbeta1-3Galbeta1-4Glcbeta-Cer(d18:1/26:1(17Z)); NeuAcalpha2-3Galbeta1-4GlcNAcbeta1-3(GlcNAcalpha1-3(Fucalpha1-2)Galbeta1-4GlcNAcb 6)Galbeta1-4GlcNAcbeta1-3Galbeta1-4Glcbeta-Cer; NeuAcalpha2-3Galbeta1-4GlcNAcbeta1-3(GlcNAcalpha1-3(Fucalpha1-2)Galbeta1-4GlcNAcbeta1-6)Galbeta1-4GlcNAcbeta1-3Galbeta1-4Glcbeta-Cer(d18:1/16:0); NeuAcalpha2-3Galbeta1-4GlcNAcbeta1-3(GlcNAcalpha1-3(Fucalpha1-2)Galbeta1-4GlcNAcbeta1-6)Galbeta1-4GlcNAcbeta1-3Galbeta1-4Glcbeta-Cer(d18:1/18:0); NeuAcalpha2-3Galbeta1-4GlcNAcbeta1-3(GlcNAcalpha1-3(Fucalpha1-2)Galbeta1-4GlcNAcbeta1-6)Galbeta1-4GlcNAcbeta1-3Galbeta1-4Glcbeta-Cer(d18:1/20:0); NeuAcalpha2-3Galbeta1-4GlcNAcbeta1-3(GlcNAcalpha1-3(Fucalpha1-2)Galbeta1-4GlcNAcbeta1-6)Galbeta1-4GlcNAcbeta1-3Galbeta1-4Glcbeta-Cer(d18:1/22:0); NeuAcalpha2-3Galbeta1-4GlcNAcbeta1-3(GlcNAcalpha1-3(Fucalpha1-2)Galbeta1-4GlcNAcbeta1-6)Galbeta1-4GlcNAcbeta1-3Galbeta1-4Glcbeta-Cer(d18:1/24:0); NeuAcalpha2-3Galbeta1-4GlcNAcbeta1-3(GlcNAcalpha1-3(Fucalpha1-2)Galbeta1-4GlcNAcbeta1-6)Galbeta1-4GlcNAcbeta1-3Galbeta1-4Glcbeta-Cer(d18:1/24:1(15Z)); NeuAcalpha2-3Galbeta1-4GlcNAcbeta1-3(GlcNAcalpha1-3(Fucalpha1-2)Galbeta1-4GlcNAcbeta1-6)Galbeta1-4GlcNAcbeta1-3Galbeta1-4Glcbeta-Cer(d18:1/26:0); NeuAcalpha2-3Galbeta1-4GlcNAcbeta1-3(GlcNAcalpha1-3(Fucalpha1-2)Galbeta1-4GlcNAcbeta1-6)Galbeta1-4GlcNAcbeta1-3Galbeta1-4Glcbeta-Cer(d18:1/26:1(17Z)); NeuAcalpha2-3Galbeta1-4GlcNAcbeta1-3(GlcNAcbeta1-6)Galbeta1-4GlcNAcbeta1-3Galbeta1-4Glcbeta-Cer; NeuAcalpha2-3Galbeta1-4GlcNAcbeta1-3(GlcNAcbeta1-6)Galbeta1-4GlcNAcbeta1-3Galbeta1-4Glcbeta-Cer(d18:1/16:0); NeuAcalpha2-3Galbeta1-4GlcNAcbeta1-3(GlcNAcbeta1-6)Galbeta1-4GlcNAcbeta1-3Galbeta1-4Glcbeta-Cer(d18:1/18:0); NeuAcalpha2-3Galbeta1-4GlcNAcbeta1-3(GlcNAcbeta1-6)Galbeta1-4GlcNAcbeta1-3Galbeta1-4Glcbeta-Cer(d18:1/20:0); NeuAcalpha2-3Galbeta1-4GlcNAcbeta1-3(GlcNAcbeta1-6)Galbeta1-4GlcNAcbeta1-3Galbeta1-4Glcbeta-Cer(d18:1/22:0); NeuAcalpha2-3Galbeta1-4GlcNAcbeta1-3(GlcNAcbeta1-6)Galbeta1-4GlcNAcbeta1-3Galbeta1-4Glcbeta-Cer(d18:1/24:0); NeuAcalpha2-3Galbeta1-4GlcNAcbeta1-3(GlcNAcbeta1-6)Galbeta1-4GlcNAcbeta1-3Galbeta1-4Glcbeta-Cer(d18:1/24:1(15Z)); NeuAcalpha2-3Galbeta1-4GlcNAcbeta1-3(GlcNAcbeta1-6)Galbeta1-4GlcNAcbeta1-3Galbeta1-4Glcbeta-Cer(d18:1/26:0); NeuAcalpha2-3Galbeta1-4GlcNAcbeta1-3(GlcNAcbeta1-6)Galbeta1-4GlcNAcbeta1-3Galbeta1-4Glcbeta-Cer(d18:1/26:1(17Z)); NeuAcalpha2-3Galbeta1-4GlcNAcbeta1-3(NeuAcalpha2-3Galbeta1-4GlcNAcbeta1-6)Galbeta1-4GlcNAcbeta1-3Galbeta1-4Glcbeta-Cer; NeuAcalpha2-3Galbeta1-4GlcNAcbeta1-3(NeuAcalpha2-3Galbeta1-4GlcNAcbeta1-6)Galbeta1-4GlcNAcbeta1-3Galbeta1-4Glcbeta-Cer(d18:1/16:0); NeuAcalpha2-3Galbeta1-4GlcNAcbeta1-3(NeuAcalpha2-3Galbeta1-4GlcNAcbeta1-6)Galbeta1-4GlcNAcbeta1-3Galbeta1-4Glcbeta-Cer(d18:1/18:0); NeuAcalpha2-3Galbeta1-4GlcNAcbeta1-3(NeuAcalpha2-3Galbeta1-4GlcNAcbeta1-6)Galbeta1-4GlcNAcbeta1-3Galbeta1-4Glcbeta-Cer(d18:1/20:0); NeuAcalpha2-3Galbeta1-4GlcNAcbeta1-3(NeuAcalpha2-3Galbeta1-4GlcNAcbeta1-6)Galbeta1-4GlcNAcbeta1-3Galbeta1-4Glcbeta-Cer(d18:1/22:0); NeuAcalpha2-3Galbeta1-4GlcNAcbeta1-3(NeuAcalpha2-3Galbeta1-4GlcNAcbeta1-6)Galbeta1-4GlcNAcbeta1-3Galbeta1-4Glcbeta-Cer(d18:1/24:0); NeuAcalpha2-3Galbeta1-4GlcNAcbeta1-3(NeuAcalpha2-3Galbeta1-4GlcNAcbeta1-6)Galbeta1-4GlcNAcbeta1-3Galbeta1-4Glcbeta-Cer(d18:1/24:1(15Z)); NeuAcalpha2-3Galbeta1-4GlcNAcbeta1-3(NeuAcalpha2-3Galbeta1-4GlcNAcbeta1-6)Galbeta1-4GlcNAcbeta1-3Galbeta1-4Glcbeta-Cer(d18:1/26:0); NeuAcalpha2-3Galbeta1-4GlcNAcbeta1-3(NeuAcalpha2-3Galbeta1-4GlcNAcbeta1-6)Galbeta1-4GlcNAcbeta1-3Galbeta1-4Glcbeta-Cer(d18:1/26:1(17Z)); NeuAcalpha2-3Galbeta1-4GlcNAcbeta1-3Galbeta1-4(Fucalpha1-3)GlcNAcbeta1-3Galbeta1-4Glcbeta-Cer; NeuAcalpha2-3Galbeta1-4GlcNAcbeta1-3Galbeta1-4(Fucalpha1-3)GlcNAcbeta1-3Galbeta1-4Glcbeta-Cer(d18:1/16:0); NeuAcalpha2-3Galbeta1-4GlcNAcbeta1-3Galbeta1-4(Fucalpha1-3)GlcNAcbeta1-3Galbeta1-4Glcbeta-Cer(d18:1/18:0); NeuAcalpha2-3Galbeta1-4GlcNAcbeta1-3Galbeta1-4(Fucalpha1-3)GlcNAcbeta1-3Galbeta1-4Glcbeta-Cer(d18:1/20:0); NeuAcalpha2-3Galbeta1-4GlcNAcbeta1-3Galbeta1-4(Fucalpha1-3)GlcNAcbeta1-3Galbeta1-4Glcbeta-Cer(d18:1/22:0); NeuAcalpha2-3Galbeta1-4GlcNAcbeta1-3Galbeta1-4(Fucalpha1-3)GlcNAcbeta1-3Galbeta1-4Glcbeta-Cer(d18:1/24:0); NeuAcalpha2-3Galbeta1-4GlcNAcbeta1-3Galbeta1-4(Fucalpha1-3)GlcNAcbeta1-3Galbeta1-4Glcbeta-Cer(d18:1/24:1(15Z)); NeuAcalpha2-3Galbeta1-4GlcNAcbeta1-3Galbeta1-4(Fucalpha1-3)GlcNAcbeta1-3Galbeta1-4Glcbeta-Cer(d18:1/26:0); NeuAcalpha2-3Galbeta1-4GlcNAcbeta1-3Galbeta1-4(Fucalpha1-3)GlcNAcbeta1-3Galbeta1-4Glcbeta-Cer(d18:1/26:1(17Z)); NeuAcalpha2-3Galbeta1-4GlcNAcbeta1-3Galbeta1-4(Fucalpha1-3)GlcNAcbeta1-3Galbeta1-4GlcNAcbeta1-3Galbeta1-4Glcbeta-Cer; NeuAcalpha2-3Galbeta1-4GlcNAcbeta1-3Galbeta1-4(Fucalpha1-3)GlcNAcbeta1-3Galbeta1-4GlcNAcbeta1-3Galbeta1-4Glcbeta-Cer(d18:1/16:0); NeuAcalpha2-3Galbeta1-4GlcNAcbeta1-3Galbeta1-4(Fucalpha1-3)GlcNAcbeta1-3Galbeta1-4GlcNAcbeta1-3Galbeta1-4Glcbeta-Cer(d18:1/18:0); NeuAcalpha2-3Galbeta1-4GlcNAcbeta1-3Galbeta1-4(Fucalpha1-3)GlcNAcbeta1-3Galbeta1-4GlcNAcbeta1-3Galbeta1-4Glcbeta-Cer(d18:1/20:0); NeuAcalpha2-3Galbeta1-4GlcNAcbeta1-3Galbeta1-4(Fucalpha1-3)GlcNAcbeta1-3Galbeta1-4GlcNAcbeta1-3Galbeta1-4Glcbeta-Cer(d18:1/22:0); NeuAcalpha2-3Galbeta1-4GlcNAcbeta1-3Galbeta1-4(Fucalpha1-3)GlcNAcbeta1-3Galbeta1-4GlcNAcbeta1-3Galbeta1-4Glcbeta-Cer(d18:1/24:0); NeuAcalpha2-3Galbeta1-4GlcNAcbeta1-3Galbeta1-4(Fucalpha1-3)GlcNAcbeta1-3Galbeta1-4GlcNAcbeta1-3Galbeta1-4Glcbeta-Cer(d18:1/24:1(15Z)); NeuAcalpha2-3Galbeta1-4GlcNAcbeta1-3Galbeta1-4(Fucalpha1-3)GlcNAcbeta1-3Galbeta1-4GlcNAcbeta1-3Galbeta1-4Glcbeta-Cer(d18:1/26:0); NeuAcalpha2-3Galbeta1-4GlcNAcbeta1-3Galbeta1-4(Fucalpha1-3)GlcNAcbeta1-3Galbeta1-4GlcNAcbeta1-3Galbeta1-4Glcbeta-Cer(d18:1/26:1(17Z)); NeuAcalpha2-3Galbeta1-4GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer; NeuAcalpha2-3Galbeta1-4GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/16:0); NeuAcalpha2-3Galbeta1-4GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/18:0); NeuAcalpha2-3Galbeta1-4GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/20:0); NeuAcalpha2-3Galbeta1-4GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/22:0); NeuAcalpha2-3Galbeta1-4GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/24:0); NeuAcalpha2-3Galbeta1-4GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/24:1(15Z)); NeuAcalpha2-3Galbeta1-4GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/26:0); NeuAcalpha2-3Galbeta1-4GlcNAcbeta1-3Galbeta1-4(Fucalpha1-3)GlcNAcbeta1-3Galbeta1-4GlcNAcbeta1-3Galbeta1-4GlcNAcbeta1-3Galbeta1-4Glcbeta-Cer(d18:1/26:1(17Z)); NeuAcalpha2-3Galbeta1-4GlcNAcbeta1-3Galbeta1-4Glcbeta-Cer; NeuAcalpha2-3Galbeta1-4GlcNAcbeta1-3Galbeta1-4Glcbeta-Cer(d18:1/16:0); NeuAcalpha2-3Galbeta1-4GlcNAcbeta1-3Galbeta1-4Glcbeta-Cer(d18:1/18:0); NeuAcalpha2-3Galbeta1-4GlcNAcbeta1-3Galbeta1-4Glcbeta-Cer(d18:1/20:0); NeuAcalpha2-3Galbeta1-4GlcNAcbeta1-3Galbeta1-4Glcbeta-Cer(d18:1/22:0); NeuAcalpha2-3Galbeta1-4GlcNAcbeta1-3Galbeta1-4Glcbeta-Cer(d18:1/24:0); NeuAcalpha2-3Galbeta1-4GlcNAcbeta1-3Galbeta1-4Glcbeta-Cer(d18:1/24:1(15Z)); NeuAcalpha2-3Galbeta1-4GlcNAcbeta1-3Galbeta1-4Glcbeta-Cer(d18:1/26:0); NeuAcalpha2-3Galbeta1-4GlcNAcbeta1-3Galbeta1-4Glcbeta-Cer(d18:1/26:1(17Z)); NeuAcalpha2-3Galbeta1-4GlcNAcbeta1-3Galbeta1-4GlcNAcbeta1-3Galbeta1-4Glcbeta-Cer; NeuAcalpha2-3Galbeta1-4GlcNAcbeta1-3Galbeta1-4GlcNAcbeta1-3Galbeta1-4Glcbeta-Cer(d18:1/16:0); NeuAcalpha2-3Galbeta1-4GlcNAcbeta1-3Galbeta1-4GlcNAcbeta1-3Galbeta1-4Glcbeta-Cer(d18:1/18:0); NeuAcalpha2-3Galbeta1-4GlcNAcbeta1-3Galbeta1-4GlcNAcbeta1-3Galbeta1-4Glcbeta-Cer(d18:1/20:0); NeuAcalpha2-3Galbeta1-4GlcNAcbeta1-3Galbeta1-4GlcNAcbeta1-3Galbeta1-4Glcbeta-Cer(d18:1/22:0); NeuAcalpha2-3Galbeta1-4GlcNAcbeta1-3Galbeta1-4GlcNAcbeta1-3Galbeta1-4Glcbeta-Cer(d18:1/24:0); NeuAcalpha2-3Galbeta1-4GlcNAcbeta1-3Galbeta1-4GlcNAcbeta1-3Galbeta1-4Glcbeta-Cer(d18:1/24:1(15Z)); NeuAcalpha2-3Galbeta1-4GlcNAcbeta1-3Galbeta1-4GlcNAcbeta1-3Galbeta1-4Glcbeta-Cer(d18:1/26:0); NeuAcalpha2-3Galbeta1-4GlcNAcbeta1-3Galbeta1-4GlcNAcbeta1-3Galbeta1-4Glcbeta-Cer(d18:1/26:1(17Z)); NeuAcalpha2-3Galbeta1-4GlcNAcbeta1-3Galbeta1-4GlcNAcbeta1-3Galbeta1-4GlcNAcbeta1-3Galbeta1-4Glcbeta-Cer; NeuAcalpha2-3Galbeta1-4GlcNAcbeta1-3Galbeta1-4GlcNAcbeta1-3Galbeta1-4GlcNAcbeta1-3Galbeta1-4Glcbeta-Cer(d18:1/16:0); NeuAcalpha2-3Galbeta1-4GlcNAcbeta1-3Galbeta1-4GlcNAcbeta1-3Galbeta1-4GlcNAcbeta1-3Galbeta1-4Glcbeta-Cer(d18:1/18:0); NeuAcalpha2-3Galbeta1-4GlcNAcbeta1-3Galbeta1-4GlcNAcbeta1-3Galbeta1-4GlcNAcbeta1-3Galbeta1-4Glcbeta-Cer(d18:1/20:0); NeuAcalpha2-3Galbeta1-4GlcNAcbeta1-3Galbeta1-4GlcNAcbeta1-3Galbeta1-4GlcNAcbeta1-3Galbeta1-4Glcbeta-Cer(d18:1/22:0); NeuAcalpha2-3Galbeta1-4GlcNAcbeta1-3Galbeta1-4GlcNAcbeta1-3Galbeta1-4GlcNAcbeta1-3Galbeta1-4Glcbeta-Cer(d18:1/24:0); NeuAcalpha2-3Galbeta1-4GlcNAcbeta1-3Galbeta1-4GlcNAcbeta1-3Galbeta1-4GlcNAcbeta1-3Galbeta1-4Glcbeta-Cer(d18:1/24:1(15Z)); NeuAcalpha2-3Galbeta1-4GlcNAcbeta1-3Galbeta1-4GlcNAcbeta1-3Galbeta1-4GlcNAcbeta1-3Galbeta1-4Glcbeta-Cer(d18:1/26:0); NeuAcalpha2-3Galbeta1-4GlcNAcbeta1-3Galbeta1-4GlcNAcbeta1-3Galbeta1-4GlcNAcbeta1-3Galbeta1-4Glcbeta-Cer(d18:1/26:1(17Z)); NeuAcalpha2-3Galbeta1-4GlcNAcbeta1-6(NeuAcbeta2-3Galbeta1-4GlcNAcbeta1-3)Galbeta1-4GlcNAcbeta1-6(NeuAcbeta2-3Galbeta1-4GlcNAcbeta1-3)Galbeta1-4GlcNAcbeta1-6(NeuAcbeta2-3Galbeta1-4GlcNAcbeta1-3)Galbeta1-4GlcNAcbeta1-3Galbeta1-4Glcbeta-Cer; NeuAcalpha2-3Galbeta1-4GlcNAcbeta1-6(NeuAcbeta2-3Galbeta1-4GlcNAcbeta1-3)Galbeta1-4GlcNAcbeta1-6(NeuAcbeta2-3Galbeta1-4GlcNAcbeta1-3)Galbeta1-4GlcNAcbeta1-6(NeuAcbeta2-3Galbeta1-4GlcNAcbeta1-3)Galbeta1-4GlcNAcbeta1-3Galbeta1-4Glcbeta-Cer(d18:1/16:0); NeuAcalpha2-3Galbeta1-4GlcNAcbeta1-6(NeuAcbeta2-3Galbeta1-4GlcNAcbeta1-3)Galbeta1-4GlcNAcbeta1-6(NeuAcbeta2-3Galbeta1-4GlcNAcbeta1-3)Galbeta1-4GlcNAcbeta1-6(NeuAcbeta2-3Galbeta1-4GlcNAcbeta1-3)Galbeta1-4GlcNAcbeta1-3Galbeta1-4Glcbeta-Cer(d18:1/18:0); NeuAcalpha2-3Galbeta1-4GlcNAcbeta1-6(NeuAcbeta2-3Galbeta1-4GlcNAcbeta1-3)Galbeta1-4GlcNAcbeta1-6(NeuAcbeta2-3Galbeta1-4GlcNAcbeta1-3)Galbeta1-4GlcNAcbeta1-6(NeuAcbeta2-3Galbeta1-4GlcNAcbeta1-3)Galbeta1-4GlcNAcbeta1-3Galbeta1-4Glcbeta-Cer(d18:1/20:0); NeuAcalpha2-3Galbeta1-4GlcNAcbeta1-6(NeuAcbeta2-3Galbeta1-4GlcNAcbeta1-3)Galbeta1-4GlcNAcbeta1-6(NeuAcbeta2-3Galbeta1-4GlcNAcbeta1-3)Galbeta1-4GlcNAcbeta1-6(NeuAcbeta2-3Galbeta1-4GlcNAcbeta1-3)Galbeta1-4GlcNAcbeta1-3Galbeta1-4Glcbeta-Cer(d18:1/22:0); NeuAcalpha2-3Galbeta1-4GlcNAcbeta1-6(NeuAcbeta2-3Galbeta1-4GlcNAcbeta1-3)Galbeta1-4GlcNAcbeta1-6(NeuAcbeta2-3Galbeta1-4GlcNAcbeta1-3)Galbeta1-4GlcNAcbeta1-6(NeuAcbeta2-3Galbeta1-4GlcNAcbeta1-3)Galbeta1-4GlcNAcbeta1-3Galbeta1-4Glcbeta-Cer(d18:1/24:0); NeuAcalpha2-3Galbeta1-4GlcNAcbeta1-6(NeuAcbeta2-3Galbeta1-4GlcNAcbeta1-3)Galbeta1-4GlcNAcbeta1-6(NeuAcbeta2-3Galbeta1-4GlcNAcbeta1-3)Galbeta1-4GlcNAcbeta1-6(NeuAcbeta2-3Galbeta1-4GlcNAcbeta1-3)Galbeta1-4GlcNAcbeta1-3Galbeta1-4Glcbeta-Cer(d18:1/24:1(15Z)); NeuAcalpha2-3Galbeta1-4GlcNAcbeta1-6(NeuAcbeta2-3Galbeta1-4GlcNAcbeta1-3)Galbeta1-4GlcNAcbeta1-6(NeuAcbeta2-3Galbeta1-4GlcNAcbeta1-3)Galbeta1-4GlcNAcbeta1-6(NeuAcbeta2-3Galbeta1-4GlcNAcbeta1-3)Galbeta1-4GlcNAcbeta1-3Galbeta1-4Glcbeta-Cer(d18:1/26:0); NeuAcalpha2-3Galbeta1-4GlcNAcbeta1-6(NeuAcbeta2-3Galbeta1-4GlcNAcbeta1-3)Galbeta1-4GlcNAcbeta1-6(NeuAcbeta2-3Galbeta1-4GlcNAcbeta1-3)Galbeta1-4GlcNAcbeta1-6(NeuAcbeta2-3Galbeta1-4GlcNAcbeta1-3)Galbeta1-4GlcNAcbeta1-3Galbeta1-4Glcbeta-Cer(d18:1/26:1(17Z)); NeuAcalpha2-3Galbeta-Cer; NeuAcalpha2-3Galbeta-Cer(d18:1/16:0); NeuAcalpha2-3Galbeta-Cer(d18:1/18:0); NeuAcalpha2-3Galbeta-Cer(d18:1/20:0); NeuAcalpha2-3Galbeta-Cer(d18:1/22:0); NeuAcalpha2-3Galbeta-Cer(d18:1/24:0); NeuAcalpha2-3Galbeta-Cer(d18:1/24:1(15Z)); NeuAcalpha2-3Galbeta-Cer(d18:1/26:0); NeuAcalpha2-3Galbeta-Cer(d18:1/26:1(17Z)); NeuAcalpha2-3GalNAcbeta1-3Galalpha1-4Galbeta1-4Glcbeta-Cer; NeuAcalpha2-3GalNAcbeta1-3Galalpha1-4Galbeta1-4Glcbeta-Cer(d18:1/16:0); NeuAcalpha2-3GalNAcbeta1-3Galalpha1-4Galbeta1-4Glcbeta-Cer(d18:1/18:0); NeuAcalpha2-3GalNAcbeta1-3Galalpha1-4Galbeta1-4Glcbeta-Cer(d18:1/20:0); NeuAcalpha2-3GalNAcbeta1-3Galalpha1-4Galbeta1-4Glcbeta-Cer(d18:1/22:0); NeuAcalpha2-3GalNAcbeta1-3Galalpha1-4Galbeta1-4Glcbeta-Cer(d18:1/24:0); NeuAcalpha2-3GalNAcbeta1-3Galalpha1-4Galbeta1-4Glcbeta-Cer(d18:1/24:1(15Z)); NeuAcalpha2-3GalNAcbeta1-3Galalpha1-4Galbeta1-4Glcbeta-Cer(d18:1/26:0); NeuAcalpha2-3GalNAcbeta1-3Galalpha1-4Galbeta1-4Glcbeta-Cer(d18:1/26:1(17Z)); NeuAcalpha2-3GalNAcbeta1-3Galbeta1-4GlcNAcbeta1-3Galbeta1-4Glcbeta-Cer; NeuAcalpha2-3GalNAcbeta1-3Galbeta1-4GlcNAcbeta1-3Galbeta1-4Glcbeta-Cer(d18:1/16:0); NeuAcalpha2-3GalNAcbeta1-3Galbeta1-4GlcNAcbeta1-3Galbeta1-4Glcbeta-Cer(d18:1/18:0); NeuAcalpha2-3GalNAcbeta1-3Galbeta1-4GlcNAcbeta1-3Galbeta1-4Glcbeta-Cer(d18:1/20:0); NeuAcalpha2-3GalNAcbeta1-3Galbeta1-4GlcNAcbeta1-3Galbeta1-4Glcbeta-Cer(d18:1/22:0); NeuAcalpha2-3GalNAcbeta1-3Galbeta1-4GlcNAcbeta1-3Galbeta1-4Glcbeta-Cer(d18:1/24:0); NeuAcalpha2-3GalNAcbeta1-3Galbeta1-4GlcNAcbeta1-3Galbeta1-4Glcbeta-Cer(d18:1/24:1(15Z)); NeuAcalpha2-3GalNAcbeta1-3Galbeta1-4GlcNAcbeta1-3Galbeta1-4Glcbeta-Cer(d18:1/26:0); NeuAcalpha2-3GalNAcbeta1-3Galbeta1-4GlcNAcbeta1-3Galbeta1-4Glcbeta-Cer(d18:1/26:1(17Z)); NeuAcalpha2-6(NeuAcalpha2-3)Galbeta1-3GalNAcbeta1-3Galalpha1-4Galbeta1-4Glcbeta-Cer; NeuAcalpha2-6(NeuAcalpha2-3)Galbeta1-3GalNAcbeta1-3Galalpha1-4Galbeta1-4Glcbeta-Cer(d18:1/16:0); NeuAcalpha2-6(NeuAcalpha2-3)Galbeta1-3GalNAcbeta1-3Galalpha1-4Galbeta1-4Glcbeta-Cer(d18:1/18:0); NeuAcalpha2-6(NeuAcalpha2-3)Galbeta1-3GalNAcbeta1-3Galalpha1-4Galbeta1-4Glcbeta-Cer(d18:1/20:0); NeuAcalpha2-6(NeuAcalpha2-3)Galbeta1-3GalNAcbeta1-3Galalpha1-4Galbeta1-4Glcbeta-Cer(d18:1/22:0); NeuAcalpha2-6(NeuAcalpha2-3)Galbeta1-3GalNAcbeta1-3Galalpha1-4Galbeta1-4Glcbeta-Cer(d18:1/24:0); NeuAcalpha2-6(NeuAcalpha2-3)Galbeta1-3GalNAcbeta1-3Galalpha1-4Galbeta1-4Glcbeta-Cer(d18:1/24:1(15Z)); NeuAcalpha2-6(NeuAcalpha2-3)Galbeta1-3GalNAcbeta1-3Galalpha1-4Galbeta1-4Glcbeta-Cer(d18:1/26:0); NeuAcalpha2-6(NeuAcalpha2-3)Galbeta1-3GalNAcbeta1-3Galalpha1-4Galbeta1-4Glcbeta-Cer(d18:1/26:1(17Z)); NeuAcalpha2-6Galbeta1-3GalNAcbeta1-3Galalpha1-3Galbeta1-4Glcbeta-Cer; NeuAcalpha2-6Galbeta1-3GalNAcbeta1-3Galalpha1-3Galbeta1-4Glcbeta-Cer(d18:1/16:0); NeuAcalpha2-6Galbeta1-3GalNAcbeta1-3Galalpha1-3Galbeta1-4Glcbeta-Cer(d18:1/18:0); NeuAcalpha2-6Galbeta1-3GalNAcbeta1-3Galalpha1-3Galbeta1-4Glcbeta-Cer(d18:1/20:0); NeuAcalpha2-6Galbeta1-3GalNAcbeta1-3Galalpha1-3Galbeta1-4Glcbeta-Cer(d18:1/22:0); NeuAcalpha2-6Galbeta1-3GalNAcbeta1-3Galalpha1-3Galbeta1-4Glcbeta-Cer(d18:1/24:0); NeuAcalpha2-6Galbeta1-3GalNAcbeta1-3Galalpha1-3Galbeta1-4Glcbeta-Cer(d18:1/24:1(15Z)); NeuAcalpha2-6Galbeta1-3GalNAcbeta1-3Galalpha1-3Galbeta1-4Glcbeta-Cer(d18:1/26:0); NeuAcalpha2-6Galbeta1-3GalNAcbeta1-3Galalpha1-3Galbeta1-4Glcbeta-Cer(d18:1/26:1(17Z)); NeuAcalpha2-6Galbeta1-3GalNAcbeta1-3Galalpha1-4Galbeta1-4Glcbeta-Cer; NeuAcalpha2-6Galbeta1-3GalNAcbeta1-3Galalpha1-4Galbeta1-4Glcbeta-Cer(d18:1/16:0); NeuAcalpha2-6Galbeta1-3GalNAcbeta1-3Galalpha1-4Galbeta1-4Glcbeta-Cer(d18:1/18:0); NeuAcalpha2-6Galbeta1-3GalNAcbeta1-3Galalpha1-4Galbeta1-4Glcbeta-Cer(d18:1/20:0); NeuAcalpha2-6Galbeta1-3GalNAcbeta1-3Galalpha1-4Galbeta1-4Glcbeta-Cer(d18:1/22:0); NeuAcalpha2-6Galbeta1-3GalNAcbeta1-3Galalpha1-4Galbeta1-4Glcbeta-Cer(d18:1/24:0); NeuAcalpha2-6Galbeta1-3GalNAcbeta1-3Galalpha1-4Galbeta1-4Glcbeta-Cer(d18:1/24:1(15Z)); NeuAcalpha2-6Galbeta1-3GalNAcbeta1-3Galalpha1-4Galbeta1-4Glcbeta-Cer(d18:1/26:0); NeuAcalpha2-6Galbeta1-3GalNAcbeta1-3Galalpha1-4Galbeta1-4Glcbeta-Cer(d18:1/26:1(17Z)); NeuAcalpha2-6Galbeta1-3GlcNAcbeta1-3Galbeta1-4Glcbeta-Cer; NeuAcalpha2-6Galbeta1-3GlcNAcbeta1-3Galbeta1-4Glcbeta-Cer(d18:1/16:0); NeuAcalpha2-6Galbeta1-3GlcNAcbeta1-3Galbeta1-4Glcbeta-Cer(d18:1/18:0); NeuAcalpha2-6Galbeta1-3GlcNAcbeta1-3Galbeta1-4Glcbeta-Cer(d18:1/20:0); NeuAcalpha2-6Galbeta1-3GlcNAcbeta1-3Galbeta1-4Glcbeta-Cer(d18:1/22:0); NeuAcalpha2-6Galbeta1-3GlcNAcbeta1-3Galbeta1-4Glcbeta-Cer(d18:1/24:0); NeuAcalpha2-6Galbeta1-3GlcNAcbeta1-3Galbeta1-4Glcbeta-Cer(d18:1/24:1(15Z)); NeuAcalpha2-6Galbeta1-3GlcNAcbeta1-3Galbeta1-4Glcbeta-Cer(d18:1/26:0); NeuAcalpha2-6Galbeta1-3GlcNAcbeta1-3Galbeta1-4Glcbeta-Cer(d18:1/26:1(17Z)); NeuAcalpha2-6Galbeta1-4GlcNAcbeta1-3(Galbeta1-4GlcNAcbeta1-6)Galbeta1-4Glcbeta-Cer; NeuAcalpha2-6Galbeta1-4GlcNAcbeta1-3(Galbeta1-4GlcNAcbeta1-6)Galbeta1-4Glcbeta-Cer(d18:1/16:0); NeuAcalpha2-6Galbeta1-4GlcNAcbeta1-3(Galbeta1-4GlcNAcbeta1-6)Galbeta1-4Glcbeta-Cer(d18:1/18:0); NeuAcalpha2-6Galbeta1-4GlcNAcbeta1-3(Galbeta1-4GlcNAcbeta1-6)Galbeta1-4Glcbeta-Cer(d18:1/20:0); NeuAcalpha2-6Galbeta1-4GlcNAcbeta1-3(Galbeta1-4GlcNAcbeta1-6)Galbeta1-4Glcbeta-Cer(d18:1/22:0); NeuAcalpha2-6Galbeta1-4GlcNAcbeta1-3(Galbeta1-4GlcNAcbeta1-6)Galbeta1-4Glcbeta-Cer(d18:1/24:0); NeuAcalpha2-6Galbeta1-4GlcNAcbeta1-3(Galbeta1-4GlcNAcbeta1-6)Galbeta1-4Glcbeta-Cer(d18:1/24:1(15Z)); NeuAcalpha2-6Galbeta1-4GlcNAcbeta1-3(Galbeta1-4GlcNAcbeta1-6)Galbeta1-4Glcbeta-Cer(d18:1/26:0); NeuAcalpha2-6Galbeta1-4GlcNAcbeta1-3(Galbeta1-4GlcNAcbeta1-6)Galbeta1-4Glcbeta-Cer(d18:1/26:1(17Z)); NeuAcalpha2-6Galbeta1-4GlcNAcbeta1-3(Galbeta1-4GlcNAcbeta1-6)Galbeta1-4GlcNAcbeta1-3Galbeta1-4Glcbeta-Cer; NeuAcalpha2-6Galbeta1-4GlcNAcbeta1-3(Galbeta1-4GlcNAcbeta1-6)Galbeta1-4GlcNAcbeta1-3Galbeta1-4Glcbeta-Cer(d18:1/16:0); NeuAcalpha2-6Galbeta1-4GlcNAcbeta1-3(Galbeta1-4GlcNAcbeta1-6)Galbeta1-4GlcNAcbeta1-3Galbeta1-4Glcbeta-Cer(d18:1/18:0); NeuAcalpha2-6Galbeta1-4GlcNAcbeta1-3(Galbeta1-4GlcNAcbeta1-6)Galbeta1-4GlcNAcbeta1-3Galbeta1-4Glcbeta-Cer(d18:1/20:0); NeuAcalpha2-6Galbeta1-4GlcNAcbeta1-3(Galbeta1-4GlcNAcbeta1-6)Galbeta1-4GlcNAcbeta1-3Galbeta1-4Glcbeta-Cer(d18:1/22:0); NeuAcalpha2-6Galbeta1-4GlcNAcbeta1-3(Galbeta1-4GlcNAcbeta1-6)Galbeta1-4GlcNAcbeta1-3Galbeta1-4Glcbeta-Cer(d18:1/24:0); NeuAcalpha2-6Galbeta1-4GlcNAcbeta1-3(Galbeta1-4GlcNAcbeta1-6)Galbeta1-4GlcNAcbeta1-3Galbeta1-4Glcbeta-Cer(d18:1/24:1(15Z)); NeuAcalpha2-6Galbeta1-4GlcNAcbeta1-3(Galbeta1-4GlcNAcbeta1-6)Galbeta1-4GlcNAcbeta1-3Galbeta1-4Glcbeta-Cer(d18:1/26:0); NeuAcalpha2-6Galbeta1-4GlcNAcbeta1-3(Galbeta1-4GlcNAcbeta1-6)Galbeta1-4GlcNAcbeta1-3Galbeta1-4Glcbeta-Cer(d18:1/26:1(17Z)); NeuAcalpha2-6Galbeta1-4GlcNAcbeta1-3(GlcNAcbeta1-6)Galbeta1-4Glcbeta-Cer; NeuAcalpha2-6Galbeta1-4GlcNAcbeta1-3(GlcNAcbeta1-6)Galbeta1-4Glcbeta-Cer(d18:1/16:0); NeuAcalpha2-6Galbeta1-4GlcNAcbeta1-3(GlcNAcbeta1-6)Galbeta1-4Glcbeta-Cer(d18:1/18:0); NeuAcalpha2-6Galbeta1-4GlcNAcbeta1-3(GlcNAcbeta1-6)Galbeta1-4Glcbeta-Cer(d18:1/20:0); NeuAcalpha2-6Galbeta1-4GlcNAcbeta1-3(GlcNAcbeta1-6)Galbeta1-4Glcbeta-Cer(d18:1/22:0); NeuAcalpha2-6Galbeta1-4GlcNAcbeta1-3(GlcNAcbeta1-6)Galbeta1-4Glcbeta-Cer(d18:1/24:0); NeuAcalpha2-6Galbeta1-4GlcNAcbeta1-3(GlcNAcbeta1-6)Galbeta1-4Glcbeta-Cer(d18:1/24:1(15Z)); NeuAcalpha2-6Galbeta1-4GlcNAcbeta1-3(GlcNAcbeta1-6)Galbeta1-4Glcbeta-Cer(d18:1/26:0); NeuAcalpha2-6Galbeta1-4GlcNAcbeta1-3(GlcNAcbeta1-6)Galbeta1-4Glcbeta-Cer(d18:1/26:1(17Z)); NeuAcalpha2-6Galbeta1-4GlcNAcbeta1-3Galbeta1-4(Fucalpha1-3)GlcNAcbeta1-3Galbeta1-4Glcbeta-Cer; NeuAcalpha2-6Galbeta1-4GlcNAcbeta1-3Galbeta1-4(Fucalpha1-3)GlcNAcbeta1-3Galbeta1-4Glcbeta-Cer(d18:1/16:0); NeuAcalpha2-6Galbeta1-4GlcNAcbeta1-3Galbeta1-4(Fucalpha1-3)GlcNAcbeta1-3Galbeta1-4Glcbeta-Cer(d18:1/18:0); NeuAcalpha2-6Galbeta1-4GlcNAcbeta1-3Galbeta1-4(Fucalpha1-3)GlcNAcbeta1-3Galbeta1-4Glcbeta-Cer(d18:1/20:0); NeuAcalpha2-6Galbeta1-4GlcNAcbeta1-3Galbeta1-4(Fucalpha1-3)GlcNAcbeta1-3Galbeta1-4Glcbeta-Cer(d18:1/22:0); NeuAcalpha2-6Galbeta1-4GlcNAcbeta1-3Galbeta1-4(Fucalpha1-3)GlcNAcbeta1-3Galbeta1-4Glcbeta-Cer(d18:1/24:0); NeuAcalpha2-6Galbeta1-4GlcNAcbeta1-3Galbeta1-4(Fucalpha1-3)GlcNAcbeta1-3Galbeta1-4Glcbeta-Cer(d18:1/24:1(15Z)); NeuAcalpha2-6Galbeta1-4GlcNAcbeta1-3Galbeta1-4(Fucalpha1-3)GlcNAcbeta1-3Galbeta1-4Glcbeta-Cer(d18:1/26:0); NeuAcalpha2-6Galbeta1-4GlcNAcbeta1-3Galbeta1-4(Fucalpha1-3)GlcNAcbeta1-3Galbeta1-4Glcbeta-Cer(d18:1/26:1(17Z)); NeuAcalpha2-6Galbeta1-4GlcNAcbeta1-3Galbeta1-4Glcbeta-Cer; NeuAcalpha2-6Galbeta1-4GlcNAcbeta1-3Galbeta1-4Glcbeta-Cer(d18:1/16:0); NeuAcalpha2-6Galbeta1-4GlcNAcbeta1-3Galbeta1-4Glcbeta-Cer(d18:1/18:0); NeuAcalpha2-6Galbeta1-4GlcNAcbeta1-3Galbeta1-4Glcbeta-Cer(d18:1/20:0); NeuAcalpha2-6Galbeta1-4GlcNAcbeta1-3Galbeta1-4Glcbeta-Cer(d18:1/22:0); NeuAcalpha2-6Galbeta1-4GlcNAcbeta1-3Galbeta1-4Glcbeta-Cer(d18:1/24:0); NeuAcalpha2-6Galbeta1-4GlcNAcbeta1-3Galbeta1-4Glcbeta-Cer(d18:1/24:1(15Z)); NeuAcalpha2-6Galbeta1-4GlcNAcbeta1-3Galbeta1-4Glcbeta-Cer(d18:1/26:0); NeuAcalpha2-6Galbeta1-4GlcNAcbeta1-3Galbeta1-4Glcbeta-Cer(d18:1/26:1(17Z)); NeuAcalpha2-6Galbeta1-4GlcNAcbeta1-3Galbeta1-4GlcNAcbeta1-3Galbeta1-4Glcbeta-Cer; NeuAcalpha2-6Galbeta1-4GlcNAcbeta1-3Galbeta1-4GlcNAcbeta1-3Galbeta1-4Glcbeta-Cer(d18:1/16:0); NeuAcalpha2-6Galbeta1-4GlcNAcbeta1-3Galbeta1-4GlcNAcbeta1-3Galbeta1-4Glcbeta-Cer(d18:1/18:0); NeuAcalpha2-6Galbeta1-4GlcNAcbeta1-3Galbeta1-4GlcNAcbeta1-3Galbeta1-4Glcbeta-Cer(d18:1/20:0); NeuAcalpha2-6Galbeta1-4GlcNAcbeta1-3Galbeta1-4GlcNAcbeta1-3Galbeta1-4Glcbeta-Cer(d18:1/22:0); NeuAcalpha2-6Galbeta1-4GlcNAcbeta1-3Galbeta1-4GlcNAcbeta1-3Galbeta1-4Glcbeta-Cer(d18:1/24:0); NeuAcalpha2-6Galbeta1-4GlcNAcbeta1-3Galbeta1-4GlcNAcbeta1-3Galbeta1-4Glcbeta-Cer(d18:1/24:1(15Z)); NeuAcalpha2-6Galbeta1-4GlcNAcbeta1-3Galbeta1-4GlcNAcbeta1-3Galbeta1-4Glcbeta-Cer(d18:1/26:0); NeuAcalpha2-6Galbeta1-4GlcNAcbeta1-3Galbeta1-4GlcNAcbeta1-3Galbeta1-4Glcbeta-Cer(d18:1/26:1(17Z)); NeuAcalpha2-6Galbeta1-4GlcNAcbeta1-3Galbeta1-4GlcNAcbeta1-3Galbeta1-4GlcNAcbeta1-3Galbeta1-4Glcbeta-Cer; NeuAcalpha2-6Galbeta1-4GlcNAcbeta1-3Galbeta1-4GlcNAcbeta1-3Galbeta1-4GlcNAcbeta1-3Galbeta1-4Glcbeta-Cer(d18:1/16:0); NeuAcalpha2-6Galbeta1-4GlcNAcbeta1-3Galbeta1-4GlcNAcbeta1-3Galbeta1-4GlcNAcbeta1-3Galbeta1-4Glcbeta-Cer(d18:1/18:0); NeuAcalpha2-6Galbeta1-4GlcNAcbeta1-3Galbeta1-4GlcNAcbeta1-3Galbeta1-4GlcNAcbeta1-3Galbeta1-4Glcbeta-Cer(d18:1/20:0); NeuAcalpha2-6Galbeta1-4GlcNAcbeta1-3Galbeta1-4GlcNAcbeta1-3Galbeta1-4GlcNAcbeta1-3Galbeta1-4Glcbeta-Cer(d18:1/22:0); NeuAcalpha2-6Galbeta1-4GlcNAcbeta1-3Galbeta1-4GlcNAcbeta1-3Galbeta1-4GlcNAcbeta1-3Galbeta1-4Glcbeta-Cer(d18:1/24:0); NeuAcalpha2-6Galbeta1-4GlcNAcbeta1-3Galbeta1-4GlcNAcbeta1-3Galbeta1-4GlcNAcbeta1-3Galbeta1-4Glcbeta-Cer(d18:1/24:1(15Z)); NeuAcalpha2-6Galbeta1-4GlcNAcbeta1-3Galbeta1-4GlcNAcbeta1-3Galbeta1-4GlcNAcbeta1-3Galbeta1-4Glcbeta-Cer(d18:1/26:0); NeuAcalpha2-6Galbeta1-4GlcNAcbeta1-3Galbeta1-4GlcNAcbeta1-3Galbeta1-4GlcNAcbeta1-3Galbeta1-4Glcbeta-Cer(d18:1/26:1(17Z)); NeuAcalpha2-6GalNAcbeta1-4Galbeta1-4Glcbeta-Cer; NeuAcalpha2-6GalNAcbeta1-4Galbeta1-4Glcbeta-Cer(d18:1/16:0); NeuAcalpha2-6GalNAcbeta1-4Galbeta1-4Glcbeta-Cer(d18:1/18:0); NeuAcalpha2-6GalNAcbeta1-4Galbeta1-4Glcbeta-Cer(d18:1/20:0); NeuAcalpha2-6GalNAcbeta1-4Galbeta1-4Glcbeta-Cer(d18:1/22:0); NeuAcalpha2-6GalNAcbeta1-4Galbeta1-4Glcbeta-Cer(d18:1/24:0); NeuAcalpha2-6GalNAcbeta1-4Galbeta1-4Glcbeta-Cer(d18:1/24:1(15Z)); NeuAcalpha2-6GalNAcbeta1-4Galbeta1-4Glcbeta-Cer(d18:1/26:0); NeuAcalpha2-6GalNAcbeta1-4Galbeta1-4Glcbeta-Cer(d18:1/26:1(17Z)); NeuAcalpha2-8NeuAcalpha2-3Galbeta1-3(NeuAcalpha2-6)GalNAcbeta1-4(NeuAcalpha2-3)Galbeta1-4Glcbeta-Cer; NeuAcalpha2-8NeuAcalpha2-3Galbeta1-3(NeuAcalpha2-6)GalNAcbeta1-4(NeuAcalpha2-3)Galbeta1-4Glcbeta-Cer(d18:1/16:0); NeuAcalpha2-8NeuAcalpha2-3Galbeta1-3(NeuAcalpha2-6)GalNAcbeta1-4(NeuAcalpha2-3)Galbeta1-4Glcbeta-Cer(d18:1/18:0); NeuAcalpha2-8NeuAcalpha2-3Galbeta1-3(NeuAcalpha2-6)GalNAcbeta1-4(NeuAcalpha2-3)Galbeta1-4Glcbeta-Cer(d18:1/20:0); NeuAcalpha2-8NeuAcalpha2-3Galbeta1-3(NeuAcalpha2-6)GalNAcbeta1-4(NeuAcalpha2-3)Galbeta1-4Glcbeta-Cer(d18:1/22:0); NeuAcalpha2-8NeuAcalpha2-3Galbeta1-3(NeuAcalpha2-6)GalNAcbeta1-4(NeuAcalpha2-3)Galbeta1-4Glcbeta-Cer(d18:1/24:0); NeuAcalpha2-8NeuAcalpha2-3Galbeta1-3(NeuAcalpha2-6)GalNAcbeta1-4(NeuAcalpha2-3)Galbeta1-4Glcbeta-Cer(d18:1/24:1(15Z)); NeuAcalpha2-8NeuAcalpha2-3Galbeta1-3(NeuAcalpha2-6)GalNAcbeta1-4(NeuAcalpha2-3)Galbeta1-4Glcbeta-Cer(d18:1/26:0); NeuAcalpha2-8NeuAcalpha2-3Galbeta1-3(NeuAcalpha2-6)GalNAcbeta1-4(NeuAcalpha2-3)Galbeta1-4Glcbeta-Cer(d18:1/26:1(17Z)); NeuAcalpha2-8NeuAcalpha2-3Galbeta1-3(NeuAcalpha2-6)GalNAcbeta1-4Galbeta1-4Glcbeta-Cer; NeuAcalpha2-8NeuAcalpha2-3Galbeta1-3(NeuAcalpha2-6)GalNAcbeta1-4Galbeta1-4Glcbeta-Cer(d18:1/16:0); NeuAcalpha2-8NeuAcalpha2-3Galbeta1-3(NeuAcalpha2-6)GalNAcbeta1-4Galbeta1-4Glcbeta-Cer(d18:1/18:0); NeuAcalpha2-8NeuAcalpha2-3Galbeta1-3(NeuAcalpha2-6)GalNAcbeta1-4Galbeta1-4Glcbeta-Cer(d18:1/20:0); NeuAcalpha2-8NeuAcalpha2-3Galbeta1-3(NeuAcalpha2-6)GalNAcbeta1-4Galbeta1-4Glcbeta-Cer(d18:1/22:0); NeuAcalpha2-8NeuAcalpha2-3Galbeta1-3(NeuAcalpha2-6)GalNAcbeta1-4Galbeta1-4Glcbeta-Cer(d18:1/24:0); NeuAcalpha2-8NeuAcalpha2-3Galbeta1-3(NeuAcalpha2-6)GalNAcbeta1-4Galbeta1-4Glcbeta-Cer(d18:1/24:1(15Z)); NeuAcalpha2-8NeuAcalpha2-3Galbeta1-3(NeuAcalpha2-6)GalNAcbeta1-4Galbeta1-4Glcbeta-Cer(d18:1/26:0); NeuAcalpha2-8NeuAcalpha2-3Galbeta1-3(NeuAcalpha2-6)GalNAcbeta1-4Galbeta1-4Glcbeta-Cer(d18:1/26:1(17Z)); NeuAcalpha2-8NeuAcalpha2-3Galbeta1-3(NeuAcalpha2-8NeuAcalpha2-6)GalNAcbeta1-4Galbeta1-4Glcbeta-Cer; NeuAcalpha2-8NeuAcalpha2-3Galbeta1-3(NeuAcalpha2-8NeuAcalpha2-6)GalNAcbeta1-4Galbeta1-4Glcbeta-Cer(d18:1/16:0); NeuAcalpha2-8NeuAcalpha2-3Galbeta1-3(NeuAcalpha2-8NeuAcalpha2-6)GalNAcbeta1-4Galbeta1-4Glcbeta-Cer(d18:1/18:0); NeuAcalpha2-8NeuAcalpha2-3Galbeta1-3(NeuAcalpha2-8NeuAcalpha2-6)GalNAcbeta1-4Galbeta1-4Glcbeta-Cer(d18:1/20:0); NeuAcalpha2-8NeuAcalpha2-3Galbeta1-3(NeuAcalpha2-8NeuAcalpha2-6)GalNAcbeta1-4Galbeta1-4Glcbeta-Cer(d18:1/22:0); NeuAcalpha2-8NeuAcalpha2-3Galbeta1-3(NeuAcalpha2-8NeuAcalpha2-6)GalNAcbeta1-4Galbeta1-4Glcbeta-Cer(d18:1/24:0); NeuAcalpha2-8NeuAcalpha2-3Galbeta1-3(NeuAcalpha2-8NeuAcalpha2-6)GalNAcbeta1-4Galbeta1-4Glcbeta-Cer(d18:1/24:1(15Z)); NeuAcalpha2-8NeuAcalpha2-3Galbeta1-3(NeuAcalpha2-8NeuAcalpha2-6)GalNAcbeta1-4Galbeta1-4Glcbeta-Cer(d18:1/26:0); NeuAcalpha2-8NeuAcalpha2-3Galbeta1-3(NeuAcalpha2-8NeuAcalpha2-6)GalNAcbeta1-4Galbeta1-4Glcbeta-Cer(d18:1/26:1(17Z)); NeuAcalpha2-8NeuAcalpha2-3Galbeta1-3GalNAcbeta1-3Galalpha1-4Galbeta1-4Glcbeta-Cer; NeuAcalpha2-8NeuAcalpha2-3Galbeta1-3GalNAcbeta1-3Galalpha1-4Galbeta1-4Glcbeta-Cer(d18:1/16:0); NeuAcalpha2-8NeuAcalpha2-3Galbeta1-3GalNAcbeta1-3Galalpha1-4Galbeta1-4Glcbeta-Cer(d18:1/18:0); NeuAcalpha2-8NeuAcalpha2-3Galbeta1-3GalNAcbeta1-3Galalpha1-4Galbeta1-4Glcbeta-Cer(d18:1/20:0); NeuAcalpha2-8NeuAcalpha2-3Galbeta1-3GalNAcbeta1-3Galalpha1-4Galbeta1-4Glcbeta-Cer(d18:1/22:0); NeuAcalpha2-8NeuAcalpha2-3Galbeta1-3GalNAcbeta1-3Galalpha1-4Galbeta1-4Glcbeta-Cer(d18:1/24:0); NeuAcalpha2-8NeuAcalpha2-3Galbeta1-3GalNAcbeta1-3Galalpha1-4Galbeta1-4Glcbeta-Cer(d18:1/24:1(15Z)); NeuAcalpha2-8NeuAcalpha2-3Galbeta1-3GalNAcbeta1-3Galalpha1-4Galbeta1-4Glcbeta-Cer(d18:1/26:0); NeuAcalpha2-8NeuAcalpha2-3Galbeta1-3GalNAcbeta1-3Galalpha1-4Galbeta1-4Glcbeta-Cer(d18:1/26:1(17Z)); NeuAcalpha2-8NeuAcalpha2-3Galbeta1-3GalNAcbeta1-4(NeuAcalpha2-3)Galbeta1-4Glcbeta-Cer; NeuAcalpha2-8NeuAcalpha2-3Galbeta1-3GalNAcbeta1-4(NeuAcalpha2-3)Galbeta1-4Glcbeta-Cer(d18:1/16:0); NeuAcalpha2-8NeuAcalpha2-3Galbeta1-3GalNAcbeta1-4(NeuAcalpha2-3)Galbeta1-4Glcbeta-Cer(d18:1/18:0); NeuAcalpha2-8NeuAcalpha2-3Galbeta1-3GalNAcbeta1-4(NeuAcalpha2-3)Galbeta1-4Glcbeta-Cer(d18:1/20:0); NeuAcalpha2-8NeuAcalpha2-3Galbeta1-3GalNAcbeta1-4(NeuAcalpha2-3)Galbeta1-4Glcbeta-Cer(d18:1/22:0); NeuAcalpha2-8NeuAcalpha2-3Galbeta1-3GalNAcbeta1-4(NeuAcalpha2-3)Galbeta1-4Glcbeta-Cer(d18:1/24:0); NeuAcalpha2-8NeuAcalpha2-3Galbeta1-3GalNAcbeta1-4(NeuAcalpha2-3)Galbeta1-4Glcbeta-Cer(d18:1/24:1(15Z)); NeuAcalpha2-8NeuAcalpha2-3Galbeta1-3GalNAcbeta1-4(NeuAcalpha2-3)Galbeta1-4Glcbeta-Cer(d18:1/26:0); NeuAcalpha2-8NeuAcalpha2-3Galbeta1-3GalNAcbeta1-4(NeuAcalpha2-3)Galbeta1-4Glcbeta-Cer(d18:1/26:1(17Z)); NeuAcalpha2-8NeuAcalpha2-3Galbeta1-3GalNAcbeta1-4(NeuAcalpha2-8NeuAcalpha2-3)Galbeta1-4Glcbeta-Cer; NeuAcalpha2-8NeuAcalpha2-3Galbeta1-3GalNAcbeta1-4(NeuAcalpha2-8NeuAcalpha2-3)Galbeta1-4Glcbeta-Cer(d18:1/16:0); NeuAcalpha2-8NeuAcalpha2-3Galbeta1-3GalNAcbeta1-4(NeuAcalpha2-8NeuAcalpha2-3)Galbeta1-4Glcbeta-Cer(d18:1/18:0); NeuAcalpha2-8NeuAcalpha2-3Galbeta1-3GalNAcbeta1-4(NeuAcalpha2-8NeuAcalpha2-3)Galbeta1-4Glcbeta-Cer(d18:1/20:0); NeuAcalpha2-8NeuAcalpha2-3Galbeta1-3GalNAcbeta1-4(NeuAcalpha2-8NeuAcalpha2-3)Galbeta1-4Glcbeta-Cer(d18:1/22:0); NeuAcalpha2-8NeuAcalpha2-3Galbeta1-3GalNAcbeta1-4(NeuAcalpha2-8NeuAcalpha2-3)Galbeta1-4Glcbeta-Cer(d18:1/24:0); NeuAcalpha2-8NeuAcalpha2-3Galbeta1-3GalNAcbeta1-4(NeuAcalpha2-8NeuAcalpha2-3)Galbeta1-4Glcbeta-Cer(d18:1/24:1(15Z)); NeuAcalpha2-8NeuAcalpha2-3Galbeta1-3GalNAcbeta1-4(NeuAcalpha2-8NeuAcalpha2-3)Galbeta1-4Glcbeta-Cer(d18:1/26:0); NeuAcalpha2-8NeuAcalpha2-3Galbeta1-3GalNAcbeta1-4(NeuAcalpha2-8NeuAcalpha2-3)Galbeta1-4Glcbeta-Cer(d18:1/26:1(17Z)); NeuAcalpha2-8NeuAcalpha2-3Galbeta1-3GalNAcbeta1-4(NeuAcalpha2-8NeuAcalpha2-8NeuAcalpha2-3)Galbeta1-4Glcbeta-Cer; NeuAcalpha2-8NeuAcalpha2-3Galbeta1-3GalNAcbeta1-4(NeuAcalpha2-8NeuAcalpha2-8NeuAcalpha2-3)Galbeta1-4Glcbeta-Cer(d18:1/16:0); NeuAcalpha2-8NeuAcalpha2-3Galbeta1-3GalNAcbeta1-4(NeuAcalpha2-8NeuAcalpha2-8NeuAcalpha2-3)Galbeta1-4Glcbeta-Cer(d18:1/18:0); NeuAcalpha2-8NeuAcalpha2-3Galbeta1-3GalNAcbeta1-4(NeuAcalpha2-8NeuAcalpha2-8NeuAcalpha2-3)Galbeta1-4Glcbeta-Cer(d18:1/20:0); NeuAcalpha2-8NeuAcalpha2-3Galbeta1-3GalNAcbeta1-4(NeuAcalpha2-8NeuAcalpha2-8NeuAcalpha2-3)Galbeta1-4Glcbeta-Cer(d18:1/22:0); NeuAcalpha2-8NeuAcalpha2-3Galbeta1-3GalNAcbeta1-4(NeuAcalpha2-8NeuAcalpha2-8NeuAcalpha2-3)Galbeta1-4Glcbeta-Cer(d18:1/24:0); NeuAcalpha2-8NeuAcalpha2-3Galbeta1-3GalNAcbeta1-4(NeuAcalpha2-8NeuAcalpha2-8NeuAcalpha2-3)Galbeta1-4Glcbeta-Cer(d18:1/24:1(15Z)); NeuAcalpha2-8NeuAcalpha2-3Galbeta1-3GalNAcbeta1-4(NeuAcalpha2-8NeuAcalpha2-8NeuAcalpha2-3)Galbeta1-4Glcbeta-Cer(d18:1/26:0); NeuAcalpha2-8NeuAcalpha2-3Galbeta1-3GalNAcbeta1-4(NeuAcalpha2-8NeuAcalpha2-8NeuAcalpha2-3)Galbeta1-4Glcbeta-Cer(d18:1/26:1(17Z)); NeuAcalpha2-8NeuAcalpha2-3Galbeta1-3GalNAcbeta1-4Galbeta1-4Glcbeta-Cer; NeuAcalpha2-8NeuAcalpha2-3Galbeta1-3GalNAcbeta1-4Galbeta1-4Glcbeta-Cer(d18:1/16:0); NeuAcalpha2-8NeuAcalpha2-3Galbeta1-3GalNAcbeta1-4Galbeta1-4Glcbeta-Cer(d18:1/18:0); NeuAcalpha2-8NeuAcalpha2-3Galbeta1-3GalNAcbeta1-4Galbeta1-4Glcbeta-Cer(d18:1/20:0); NeuAcalpha2-8NeuAcalpha2-3Galbeta1-3GalNAcbeta1-4Galbeta1-4Glcbeta-Cer(d18:1/22:0); NeuAcalpha2-8NeuAcalpha2-3Galbeta1-3GalNAcbeta1-4Galbeta1-4Glcbeta-Cer(d18:1/24:0); NeuAcalpha2-8NeuAcalpha2-3Galbeta1-3GalNAcbeta1-4Galbeta1-4Glcbeta-Cer(d18:1/24:1(15Z)); NeuAcalpha2-8NeuAcalpha2-3Galbeta1-3GalNAcbeta1-4Galbeta1-4Glcbeta-Cer(d18:1/26:0); NeuAcalpha2-8NeuAcalpha2-3Galbeta1-3GalNAcbeta1-4Galbeta1-4Glcbeta-Cer(d18:1/26:1(17Z)); NeuAcalpha2-8NeuAcalpha2-3Galbeta1-3GlcNAcbeta1-3Galbeta1-4Glcbeta-Cer; NeuAcalpha2-8NeuAcalpha2-3Galbeta1-3GlcNAcbeta1-3Galbeta1-4Glcbeta-Cer(d18:1/16:0); NeuAcalpha2-8NeuAcalpha2-3Galbeta1-3GlcNAcbeta1-3Galbeta1-4Glcbeta-Cer(d18:1/18:0); NeuAcalpha2-8NeuAcalpha2-3Galbeta1-3GlcNAcbeta1-3Galbeta1-4Glcbeta-Cer(d18:1/20:0); NeuAcalpha2-8NeuAcalpha2-3Galbeta1-3GlcNAcbeta1-3Galbeta1-4Glcbeta-Cer(d18:1/22:0); NeuAcalpha2-8NeuAcalpha2-3Galbeta1-3GlcNAcbeta1-3Galbeta1-4Glcbeta-Cer(d18:1/24:0); NeuAcalpha2-8NeuAcalpha2-3Galbeta1-3GlcNAcbeta1-3Galbeta1-4Glcbeta-Cer(d18:1/24:1(15Z)); NeuAcalpha2-8NeuAcalpha2-3Galbeta1-3GlcNAcbeta1-3Galbeta1-4Glcbeta-Cer(d18:1/26:0); NeuAcalpha2-8NeuAcalpha2-3Galbeta1-3GlcNAcbeta1-3Galbeta1-4Glcbeta-Cer(d18:1/26:1(17Z)); NeuAcalpha2-8NeuAcalpha2-3Galbeta1-4Glcbeta-Cer; NeuAcalpha2-8NeuAcalpha2-3Galbeta1-4Glcbeta-Cer(d18:1/16:0); NeuAcalpha2-8NeuAcalpha2-3Galbeta1-4Glcbeta-Cer(d18:1/18:0); NeuAcalpha2-8NeuAcalpha2-3Galbeta1-4Glcbeta-Cer(d18:1/20:0); NeuAcalpha2-8NeuAcalpha2-3Galbeta1-4Glcbeta-Cer(d18:1/22:0); NeuAcalpha2-8NeuAcalpha2-3Galbeta1-4Glcbeta-Cer(d18:1/24:0); NeuAcalpha2-8NeuAcalpha2-3Galbeta1-4Glcbeta-Cer(d18:1/24:1(15Z)); NeuAcalpha2-8NeuAcalpha2-3Galbeta1-4Glcbeta-Cer(d18:1/26:0); NeuAcalpha2-8NeuAcalpha2-3Galbeta1-4Glcbeta-Cer(d18:1/26:1(17Z)); NeuAcalpha2-8NeuAcalpha2-3Galbeta1-4GlcNAcbeta1-3Galbeta1-4Glcbeta-Cer; NeuAcalpha2-8NeuAcalpha2-3Galbeta1-4GlcNAcbeta1-3Galbeta1-4Glcbeta-Cer(d18:1/16:0); NeuAcalpha2-8NeuAcalpha2-3Galbeta1-4GlcNAcbeta1-3Galbeta1-4Glcbeta-Cer(d18:1/18:0); NeuAcalpha2-8NeuAcalpha2-3Galbeta1-4GlcNAcbeta1-3Galbeta1-4Glcbeta-Cer(d18:1/20:0); NeuAcalpha2-8NeuAcalpha2-3Galbeta1-4GlcNAcbeta1-3Galbeta1-4Glcbeta-Cer(d18:1/22:0); NeuAcalpha2-8NeuAcalpha2-3Galbeta1-4GlcNAcbeta1-3Galbeta1-4Glcbeta-Cer(d18:1/24:0); NeuAcalpha2-8NeuAcalpha2-3Galbeta1-4GlcNAcbeta1-3Galbeta1-4Glcbeta-Cer(d18:1/24:1(15Z)); NeuAcalpha2-8NeuAcalpha2-3Galbeta1-4GlcNAcbeta1-3Galbeta1-4Glcbeta-Cer(d18:1/26:0); NeuAcalpha2-8NeuAcalpha2-3Galbeta1-4GlcNAcbeta1-3Galbeta1-4Glcbeta-Cer(d18:1/26:1(17Z)); NeuAcalpha2-8NeuAcalpha2-3Galbeta1-4GlcNAcbeta1-3Galbeta1-4GlcNAcbeta1-3Galbeta1-4Glcbeta-Cer; NeuAcalpha2-8NeuAcalpha2-3Galbeta1-4GlcNAcbeta1-3Galbeta1-4GlcNAcbeta1-3Galbeta1-4Glcbeta-Cer(d18:1/16:0); NeuAcalpha2-8NeuAcalpha2-3Galbeta1-4GlcNAcbeta1-3Galbeta1-4GlcNAcbeta1-3Galbeta1-4Glcbeta-Cer(d18:1/18:0); NeuAcalpha2-8NeuAcalpha2-3Galbeta1-4GlcNAcbeta1-3Galbeta1-4GlcNAcbeta1-3Galbeta1-4Glcbeta-Cer(d18:1/20:0); NeuAcalpha2-8NeuAcalpha2-3Galbeta1-4GlcNAcbeta1-3Galbeta1-4GlcNAcbeta1-3Galbeta1-4Glcbeta-Cer(d18:1/22:0); NeuAcalpha2-8NeuAcalpha2-3Galbeta1-4GlcNAcbeta1-3Galbeta1-4GlcNAcbeta1-3Galbeta1-4Glcbeta-Cer(d18:1/24:0); NeuAcalpha2-8NeuAcalpha2-3Galbeta1-4GlcNAcbeta1-3Galbeta1-4GlcNAcbeta1-3Galbeta1-4Glcbeta-Cer(d18:1/24:1(15Z)); NeuAcalpha2-8NeuAcalpha2-3Galbeta1-4GlcNAcbeta1-3Galbeta1-4GlcNAcbeta1-3Galbeta1-4Glcbeta-Cer(d18:1/26:0); NeuAcalpha2-8NeuAcalpha2-3Galbeta1-4GlcNAcbeta1-3Galbeta1-4GlcNAcbeta1-3Galbeta1-4Glcbeta-Cer(d18:1/26:1(17Z)); NeuAcalpha2-8NeuAcalpha2-8NeuAcalpha2-3Galbeta1-4Glcbeta-Cer; NeuAcalpha2-8NeuAcalpha2-8NeuAcalpha2-3Galbeta1-4Glcbeta-Cer(d18:1/16:0); NeuAcalpha2-8NeuAcalpha2-8NeuAcalpha2-3Galbeta1-4Glcbeta-Cer(d18:1/18:0); NeuAcalpha2-8NeuAcalpha2-8NeuAcalpha2-3Galbeta1-4Glcbeta-Cer(d18:1/20:0); NeuAcalpha2-8NeuAcalpha2-8NeuAcalpha2-3Galbeta1-4Glcbeta-Cer(d18:1/22:0); NeuAcalpha2-8NeuAcalpha2-8NeuAcalpha2-3Galbeta1-4Glcbeta-Cer(d18:1/24:0); NeuAcalpha2-8NeuAcalpha2-8NeuAcalpha2-3Galbeta1-4Glcbeta-Cer(d18:1/24:1(15Z)); NeuAcalpha2-8NeuAcalpha2-8NeuAcalpha2-3Galbeta1-4Glcbeta-Cer(d18:1/26:0); NeuAcalpha2-8NeuAcalpha2-8NeuAcalpha2-3Galbeta1-4Glcbeta-Cer(d18:1/26:1(17Z)); NeuAcalpha2-8NeuAcalpha2-8NeuAcalpha2-3Galbeta1-4GlcNAcbeta1-3Galbeta1-4Glcbeta-Cer; NeuAcalpha2-8NeuAcalpha2-8NeuAcalpha2-3Galbeta1-4GlcNAcbeta1-3Galbeta1-4Glcbeta-Cer(d18:1/16:0); NeuAcalpha2-8NeuAcalpha2-8NeuAcalpha2-3Galbeta1-4GlcNAcbeta1-3Galbeta1-4Glcbeta-Cer(d18:1/18:0); NeuAcalpha2-8NeuAcalpha2-8NeuAcalpha2-3Galbeta1-4GlcNAcbeta1-3Galbeta1-4Glcbeta-Cer(d18:1/20:0); NeuAcalpha2-8NeuAcalpha2-8NeuAcalpha2-3Galbeta1-4GlcNAcbeta1-3Galbeta1-4Glcbeta-Cer(d18:1/22:0); NeuAcalpha2-8NeuAcalpha2-8NeuAcalpha2-3Galbeta1-4GlcNAcbeta1-3Galbeta1-4Glcbeta-Cer(d18:1/24:0); NeuAcalpha2-8NeuAcalpha2-8NeuAcalpha2-3Galbeta1-4GlcNAcbeta1-3Galbeta1-4Glcbeta-Cer(d18:1/24:1(15Z)); NeuAcalpha2-8NeuAcalpha2-8NeuAcalpha2-3Galbeta1-4GlcNAcbeta1-3Galbeta1-4Glcbeta-Cer(d18:1/26:0); NeuAcalpha2-8NeuAcalpha2-8NeuAcalpha2-3Galbeta1-4GlcNAcbeta1-3Galbeta1-4Glcbeta-Cer(d18:1/26:1(17Z)); NeuAcalpha2-8NeuGcalpha2-3Galbeta1-4GlcNAcbeta1-3Galbeta1-4Glcbeta-Cer; NeuAcalpha2-8NeuGcalpha2-3Galbeta1-4GlcNAcbeta1-3Galbeta1-4Glcbeta-Cer(d18:1/16:0); NeuAcalpha2-8NeuGcalpha2-3Galbeta1-4GlcNAcbeta1-3Galbeta1-4Glcbeta-Cer(d18:1/18:0); NeuAcalpha2-8NeuGcalpha2-3Galbeta1-4GlcNAcbeta1-3Galbeta1-4Glcbeta-Cer(d18:1/20:0); NeuAcalpha2-8NeuGcalpha2-3Galbeta1-4GlcNAcbeta1-3Galbeta1-4Glcbeta-Cer(d18:1/22:0); NeuAcalpha2-8NeuGcalpha2-3Galbeta1-4GlcNAcbeta1-3Galbeta1-4Glcbeta-Cer(d18:1/24:0); NeuAcalpha2-8NeuGcalpha2-3Galbeta1-4GlcNAcbeta1-3Galbeta1-4Glcbeta-Cer(d18:1/24:1(15Z)); NeuAcalpha2-8NeuGcalpha2-3Galbeta1-4GlcNAcbeta1-3Galbeta1-4Glcbeta-Cer(d18:1/26:0); NeuAcalpha2-8NeuGcalpha2-3Galbeta1-4GlcNAcbeta1-3Galbeta1-4Glcbeta-Cer(d18:1/26:1(17Z)); NeuGcalpha2-3Galbeta1-3GalNAcbeta1-3Galalpha1-4Galbeta1-4Glcbeta-Cer; NeuGcalpha2-3Galbeta1-3GalNAcbeta1-3Galalpha1-4Galbeta1-4Glcbeta-Cer(d18:1/16:0); NeuGcalpha2-3Galbeta1-3GalNAcbeta1-3Galalpha1-4Galbeta1-4Glcbeta-Cer(d18:1/18:0); NeuGcalpha2-3Galbeta1-3GalNAcbeta1-3Galalpha1-4Galbeta1-4Glcbeta-Cer(d18:1/20:0); NeuGcalpha2-3Galbeta1-3GalNAcbeta1-3Galalpha1-4Galbeta1-4Glcbeta-Cer(d18:1/22:0); NeuGcalpha2-3Galbeta1-3GalNAcbeta1-3Galalpha1-4Galbeta1-4Glcbeta-Cer(d18:1/24:0); NeuGcalpha2-3Galbeta1-3GalNAcbeta1-3Galalpha1-4Galbeta1-4Glcbeta-Cer(d18:1/24:1(15Z)); NeuGcalpha2-3Galbeta1-3GalNAcbeta1-3Galalpha1-4Galbeta1-4Glcbeta-Cer(d18:1/26:0); NeuGcalpha2-3Galbeta1-3GalNAcbeta1-3Galalpha1-4Galbeta1-4Glcbeta-Cer(d18:1/26:1(17Z)); NeuGcalpha2-3Galbeta1-3GalNAcbeta1-4(NeuAcalpha2-3)Galbeta1-4Glcbeta-Cer; NeuGcalpha2-3Galbeta1-3GalNAcbeta1-4(NeuAcalpha2-3)Galbeta1-4Glcbeta-Cer(d18:1/16:0); NeuGcalpha2-3Galbeta1-3GalNAcbeta1-4(NeuAcalpha2-3)Galbeta1-4Glcbeta-Cer(d18:1/18:0); NeuGcalpha2-3Galbeta1-3GalNAcbeta1-4(NeuAcalpha2-3)Galbeta1-4Glcbeta-Cer(d18:1/20:0); NeuGcalpha2-3Galbeta1-3GalNAcbeta1-4(NeuAcalpha2-3)Galbeta1-4Glcbeta-Cer(d18:1/22:0); NeuGcalpha2-3Galbeta1-3GalNAcbeta1-4(NeuAcalpha2-3)Galbeta1-4Glcbeta-Cer(d18:1/24:0); NeuGcalpha2-3Galbeta1-3GalNAcbeta1-4(NeuAcalpha2-3)Galbeta1-4Glcbeta-Cer(d18:1/24:1(15Z)); NeuGcalpha2-3Galbeta1-3GalNAcbeta1-4(NeuAcalpha2-3)Galbeta1-4Glcbeta-Cer(d18:1/26:0); NeuGcalpha2-3Galbeta1-3GalNAcbeta1-4(NeuAcalpha2-3)Galbeta1-4Glcbeta-Cer(d18:1/26:1(17Z)); NeuGcalpha2-3Galbeta1-3GalNAcbeta1-4(NeuGcalpha2-3)Galbeta1-4Glcbeta-Cer; NeuGcalpha2-3Galbeta1-3GalNAcbeta1-4(NeuGcalpha2-3)Galbeta1-4Glcbeta-Cer(d18:1/16:0); NeuGcalpha2-3Galbeta1-3GalNAcbeta1-4(NeuGcalpha2-3)Galbeta1-4Glcbeta-Cer(d18:1/18:0); NeuGcalpha2-3Galbeta1-3GalNAcbeta1-4(NeuGcalpha2-3)Galbeta1-4Glcbeta-Cer(d18:1/20:0); NeuGcalpha2-3Galbeta1-3GalNAcbeta1-4(NeuGcalpha2-3)Galbeta1-4Glcbeta-Cer(d18:1/22:0); NeuGcalpha2-3Galbeta1-3GalNAcbeta1-4(NeuGcalpha2-3)Galbeta1-4Glcbeta-Cer(d18:1/24:0); NeuGcalpha2-3Galbeta1-3GalNAcbeta1-4(NeuGcalpha2-3)Galbeta1-4Glcbeta-Cer(d18:1/24:1(15Z)); NeuGcalpha2-3Galbeta1-3GalNAcbeta1-4(NeuGcalpha2-3)Galbeta1-4Glcbeta-Cer(d18:1/26:0); NeuGcalpha2-3Galbeta1-3GalNAcbeta1-4(NeuGcalpha2-3)Galbeta1-4Glcbeta-Cer(d18:1/26:1(17Z)); NeuGcalpha2-3Galbeta1-3GalNAcbeta1-4Galbeta1-4Glcbeta-Cer; NeuGcalpha2-3Galbeta1-3GalNAcbeta1-4Galbeta1-4Glcbeta-Cer(d18:1/16:0); NeuGcalpha2-3Galbeta1-3GalNAcbeta1-4Galbeta1-4Glcbeta-Cer(d18:1/18:0); NeuGcalpha2-3Galbeta1-3GalNAcbeta1-4Galbeta1-4Glcbeta-Cer(d18:1/20:0); NeuGcalpha2-3Galbeta1-3GalNAcbeta1-4Galbeta1-4Glcbeta-Cer(d18:1/22:0); NeuGcalpha2-3Galbeta1-3GalNAcbeta1-4Galbeta1-4Glcbeta-Cer(d18:1/24:0); NeuGcalpha2-3Galbeta1-3GalNAcbeta1-4Galbeta1-4Glcbeta-Cer(d18:1/24:1(15Z)); NeuGcalpha2-3Galbeta1-3GalNAcbeta1-4Galbeta1-4Glcbeta-Cer(d18:1/26:0); NeuGcalpha2-3Galbeta1-3GalNAcbeta1-4Galbeta1-4Glcbeta-Cer(d18:1/26:1(17Z)); NeuGcalpha2-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer; NeuGcalpha2-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/16:0); NeuGcalpha2-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/18:0); NeuGcalpha2-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/20:0); NeuGcalpha2-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/22:0); NeuGcalpha2-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:0); NeuGcalpha2-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/24:1(15Z)); NeuGcalpha2-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:0); NeuGcalpha2-3Galbeta1-4GlcNAcbeta1-3(Galalpha1-3Galbeta1-4GlcNAcbeta1-6)Galbeta1-4GlcNAcbeta1-3Galbeta1-4Glcbeta-Cer(d18:1/26:1(17Z)); NeuGcalpha2-3Galbeta1-4GlcNAcbeta1-3(Galbeta1-4GlcNAcbeta1-6)Galbeta1-4GlcNAcbeta1-3Galbeta1-4Glcbeta-Cer; NeuGcalpha2-3Galbeta1-4GlcNAcbeta1-3(Galbeta1-4GlcNAcbeta1-6)Galbeta1-4GlcNAcbeta1-3Galbeta1-4Glcbeta-Cer(d18:1/16:0); NeuGcalpha2-3Galbeta1-4GlcNAcbeta1-3(Galbeta1-4GlcNAcbeta1-6)Galbeta1-4GlcNAcbeta1-3Galbeta1-4Glcbeta-Cer(d18:1/18:0); NeuGcalpha2-3Galbeta1-4GlcNAcbeta1-3(Galbeta1-4GlcNAcbeta1-6)Galbeta1-4GlcNAcbeta1-3Galbeta1-4Glcbeta-Cer(d18:1/20:0); NeuGcalpha2-3Galbeta1-4GlcNAcbeta1-3(Galbeta1-4GlcNAcbeta1-6)Galbeta1-4GlcNAcbeta1-3Galbeta1-4Glcbeta-Cer(d18:1/22:0); NeuGcalpha2-3Galbeta1-4GlcNAcbeta1-3(Galbeta1-4GlcNAcbeta1-6)Galbeta1-4GlcNAcbeta1-3Galbeta1-4Glcbeta-Cer(d18:1/24:0); NeuGcalpha2-3Galbeta1-4GlcNAcbeta1-3(Galbeta1-4GlcNAcbeta1-6)Galbeta1-4GlcNAcbeta1-3Galbeta1-4Glcbeta-Cer(d18:1/24:1(15Z)); NeuGcalpha2-3Galbeta1-4GlcNAcbeta1-3(Galbeta1-4GlcNAcbeta1-6)Galbeta1-4GlcNAcbeta1-3Galbeta1-4Glcbeta-Cer(d18:1/26:0); NeuGcalpha2-3Galbeta1-4GlcNAcbeta1-3(Galbeta1-4GlcNAcbeta1-6)Galbeta1-4GlcNAcbeta1-3Galbeta1-4Glcbeta-Cer(d18:1/26:1(17Z)); NeuGcalpha2-3Galbeta1-4GlcNAcbeta1-3(GlcNAcbeta1-6)Galbeta1-4GlcNAcbeta1-3Galbeta1-4Glcbeta-Cer; NeuGcalpha2-3Galbeta1-4GlcNAcbeta1-3(GlcNAcbeta1-6)Galbeta1-4GlcNAcbeta1-3Galbeta1-4Glcbeta-Cer(d18:1/16:0); NeuGcalpha2-3Galbeta1-4GlcNAcbeta1-3(GlcNAcbeta1-6)Galbeta1-4GlcNAcbeta1-3Galbeta1-4Glcbeta-Cer(d18:1/18:0); NeuGcalpha2-3Galbeta1-4GlcNAcbeta1-3(GlcNAcbeta1-6)Galbeta1-4GlcNAcbeta1-3Galbeta1-4Glcbeta-Cer(d18:1/20:0); NeuGcalpha2-3Galbeta1-4GlcNAcbeta1-3(GlcNAcbeta1-6)Galbeta1-4GlcNAcbeta1-3Galbeta1-4Glcbeta-Cer(d18:1/22:0); NeuGcalpha2-3Galbeta1-4GlcNAcbeta1-3(GlcNAcbeta1-6)Galbeta1-4GlcNAcbeta1-3Galbeta1-4Glcbeta-Cer(d18:1/24:0); NeuGcalpha2-3Galbeta1-4GlcNAcbeta1-3(GlcNAcbeta1-6)Galbeta1-4GlcNAcbeta1-3Galbeta1-4Glcbeta-Cer(d18:1/24:1(15Z)); NeuGcalpha2-3Galbeta1-4GlcNAcbeta1-3(GlcNAcbeta1-6)Galbeta1-4GlcNAcbeta1-3Galbeta1-4Glcbeta-Cer(d18:1/26:0); NeuGcalpha2-3Galbeta1-4GlcNAcbeta1-3(GlcNAcbeta1-6)Galbeta1-4GlcNAcbeta1-3Galbeta1-4Glcbeta-Cer(d18:1/26:1(17Z)); NeuGcalpha2-3Galbeta1-4GlcNAcbeta1-3Galbeta1-3GalNAcbeta1-4(NeuGcalpha2-3)Galbeta1-4Glcbeta-Cer; NeuGcalpha2-3Galbeta1-4GlcNAcbeta1-3Galbeta1-3GalNAcbeta1-4(NeuGcalpha2-3)Galbeta1-4Glcbeta-Cer(d18:1/16:0); NeuGcalpha2-3Galbeta1-4GlcNAcbeta1-3Galbeta1-3GalNAcbeta1-4(NeuGcalpha2-3)Galbeta1-4Glcbeta-Cer(d18:1/18:0); NeuGcalpha2-3Galbeta1-4GlcNAcbeta1-3Galbeta1-3GalNAcbeta1-4(NeuGcalpha2-3)Galbeta1-4Glcbeta-Cer(d18:1/20:0); NeuGcalpha2-3Galbeta1-4GlcNAcbeta1-3Galbeta1-3GalNAcbeta1-4(NeuGcalpha2-3)Galbeta1-4Glcbeta-Cer(d18:1/22:0); NeuGcalpha2-3Galbeta1-4GlcNAcbeta1-3Galbeta1-3GalNAcbeta1-4(NeuGcalpha2-3)Galbeta1-4Glcbeta-Cer(d18:1/24:0); NeuGcalpha2-3Galbeta1-4GlcNAcbeta1-3Galbeta1-3GalNAcbeta1-4(NeuGcalpha2-3)Galbeta1-4Glcbeta-Cer(d18:1/24:1(15Z)); NeuGcalpha2-3Galbeta1-4GlcNAcbeta1-3Galbeta1-3GalNAcbeta1-4(NeuGcalpha2-3)Galbeta1-4Glcbeta-Cer(d18:1/26:0); NeuGcalpha2-3Galbeta1-4GlcNAcbeta1-3Galbeta1-3GalNAcbeta1-4(NeuGcalpha2-3)Galbeta1-4Glcbeta-Cer(d18:1/26:1(17Z)); NeuGcalpha2-3Galbeta1-4GlcNAcbeta1-3Galbeta1-3GalNAcbeta1-4Galbeta1-4Glcbeta-Cer; NeuGcalpha2-3Galbeta1-4GlcNAcbeta1-3Galbeta1-3GalNAcbeta1-4Galbeta1-4Glcbeta-Cer(d18:1/16:0); NeuGcalpha2-3Galbeta1-4GlcNAcbeta1-3Galbeta1-3GalNAcbeta1-4Galbeta1-4Glcbeta-Cer(d18:1/18:0); NeuGcalpha2-3Galbeta1-4GlcNAcbeta1-3Galbeta1-3GalNAcbeta1-4Galbeta1-4Glcbeta-Cer(d18:1/20:0); NeuGcalpha2-3Galbeta1-4GlcNAcbeta1-3Galbeta1-3GalNAcbeta1-4Galbeta1-4Glcbeta-Cer(d18:1/22:0); NeuGcalpha2-3Galbeta1-4GlcNAcbeta1-3Galbeta1-3GalNAcbeta1-4Galbeta1-4Glcbeta-Cer(d18:1/24:0); NeuGcalpha2-3Galbeta1-4GlcNAcbeta1-3Galbeta1-3GalNAcbeta1-4Galbeta1-4Glcbeta-Cer(d18:1/24:1(15Z)); NeuGcalpha2-3Galbeta1-4GlcNAcbeta1-3Galbeta1-3GalNAcbeta1-4Galbeta1-4Glcbeta-Cer(d18:1/26:0); NeuGcalpha2-3Galbeta1-4GlcNAcbeta1-3Galbeta1-3GalNAcbeta1-4Galbeta1-4Glcbeta-Cer(d18:1/26:1(17Z)); NeuGcalpha2-3Galbeta1-4GlcNAcbeta1-3Galbeta1-4Glcbeta-Cer; NeuGcalpha2-3Galbeta1-4GlcNAcbeta1-3Galbeta1-4Glcbeta-Cer(d18:1/16:0); NeuGcalpha2-3Galbeta1-4GlcNAcbeta1-3Galbeta1-4Glcbeta-Cer(d18:1/18:0); NeuGcalpha2-3Galbeta1-4GlcNAcbeta1-3Galbeta1-4Glcbeta-Cer(d18:1/20:0); NeuGcalpha2-3Galbeta1-4GlcNAcbeta1-3Galbeta1-4Glcbeta-Cer(d18:1/22:0); NeuGcalpha2-3Galbeta1-4GlcNAcbeta1-3Galbeta1-4Glcbeta-Cer(d18:1/24:0); NeuGcalpha2-3Galbeta1-4GlcNAcbeta1-3Galbeta1-4Glcbeta-Cer(d18:1/24:1(15Z)); NeuGcalpha2-3Galbeta1-4GlcNAcbeta1-3Galbeta1-4Glcbeta-Cer(d18:1/26:0); NeuGcalpha2-3Galbeta1-4GlcNAcbeta1-3Galbeta1-4Glcbeta-Cer(d18:1/26:1(17Z)); NeuGcalpha2-3Galbeta1-4GlcNAcbeta1-3Galbeta1-4GlcNAcbeta1-3Galbeta1-4Glcbeta-Cer; NeuGcalpha2-3Galbeta1-4GlcNAcbeta1-3Galbeta1-4GlcNAcbeta1-3Galbeta1-4Glcbeta-Cer(d18:1/16:0); NeuGcalpha2-3Galbeta1-4GlcNAcbeta1-3Galbeta1-4GlcNAcbeta1-3Galbeta1-4Glcbeta-Cer(d18:1/18:0); NeuGcalpha2-3Galbeta1-4GlcNAcbeta1-3Galbeta1-4GlcNAcbeta1-3Galbeta1-4Glcbeta-Cer(d18:1/20:0); NeuGcalpha2-3Galbeta1-4GlcNAcbeta1-3Galbeta1-4GlcNAcbeta1-3Galbeta1-4Glcbeta-Cer(d18:1/22:0); NeuGcalpha2-3Galbeta1-4GlcNAcbeta1-3Galbeta1-4GlcNAcbeta1-3Galbeta1-4Glcbeta-Cer(d18:1/24:0); NeuGcalpha2-3Galbeta1-4GlcNAcbeta1-3Galbeta1-4GlcNAcbeta1-3Galbeta1-4Glcbeta-Cer(d18:1/24:1(15Z)); NeuGcalpha2-3Galbeta1-4GlcNAcbeta1-3Galbeta1-4GlcNAcbeta1-3Galbeta1-4Glcbeta-Cer(d18:1/26:0); NeuGcalpha2-3Galbeta1-4GlcNAcbeta1-3Galbeta1-4GlcNAcbeta1-3Galbeta1-4Glcbeta-Cer(d18:1/26:1(17Z)); NeuGcalpha2-8NeuGcalpha2-3Galbeta1-3GalNAcbeta1-3Galalpha1-4Galbeta1-4Glcbeta-Cer; NeuGcalpha2-8NeuGcalpha2-3Galbeta1-3GalNAcbeta1-3Galalpha1-4Galbeta1-4Glcbeta-Cer(d18:1/16:0); NeuGcalpha2-8NeuGcalpha2-3Galbeta1-3GalNAcbeta1-3Galalpha1-4Galbeta1-4Glcbeta-Cer(d18:1/18:0); NeuGcalpha2-8NeuGcalpha2-3Galbeta1-3GalNAcbeta1-3Galalpha1-4Galbeta1-4Glcbeta-Cer(d18:1/20:0); NeuGcalpha2-8NeuGcalpha2-3Galbeta1-3GalNAcbeta1-3Galalpha1-4Galbeta1-4Glcbeta-Cer(d18:1/22:0); NeuGcalpha2-8NeuGcalpha2-3Galbeta1-3GalNAcbeta1-3Galalpha1-4Galbeta1-4Glcbeta-Cer(d18:1/24:0); NeuGcalpha2-8NeuGcalpha2-3Galbeta1-3GalNAcbeta1-3Galalpha1-4Galbeta1-4Glcbeta-Cer(d18:1/24:1(15Z)); NeuGcalpha2-8NeuGcalpha2-3Galbeta1-3GalNAcbeta1-3Galalpha1-4Galbeta1-4Glcbeta-Cer(d18:1/26:0); NeuGcalpha2-8NeuGcalpha2-3Galbeta1-3GalNAcbeta1-3Galalpha1-4Galbeta1-4Glcbeta-Cer(d18:1/26:1(17Z)); NeuGcalpha2-8NeuGcalpha2-3Galbeta1-4GlcNAcbeta1-3Galbeta1-4Glcbeta-Cer; NeuGcalpha2-8NeuGcalpha2-3Galbeta1-4GlcNAcbeta1-3Galbeta1-4Glcbeta-Cer(d18:1/16:0); NeuGcalpha2-8NeuGcalpha2-3Galbeta1-4GlcNAcbeta1-3Galbeta1-4Glcbeta-Cer(d18:1/18:0); NeuGcalpha2-8NeuGcalpha2-3Galbeta1-4GlcNAcbeta1-3Galbeta1-4Glcbeta-Cer(d18:1/20:0); NeuGcalpha2-8NeuGcalpha2-3Galbeta1-4GlcNAcbeta1-3Galbeta1-4Glcbeta-Cer(d18:1/22:0); NeuGcalpha2-8NeuGcalpha2-3Galbeta1-4GlcNAcbeta1-3Galbeta1-4Glcbeta-Cer(d18:1/24:0); NeuGcalpha2-8NeuGcalpha2-3Galbeta1-4GlcNAcbeta1-3Galbeta1-4Glcbeta-Cer(d18:1/24:1(15Z)); NeuGcalpha2-8NeuGcalpha2-3Galbeta1-4GlcNAcbeta1-3Galbeta1-4Glcbeta-Cer(d18:1/26:0); NeuGcalpha2-8NeuGcalpha2-3Galbeta1-4GlcNAcbeta1-3Galbeta1-4Glcbeta-Cer(d18:1/26:1(17Z)); N-hexacosanoyl)-4R-hydroxysphinganine-1-O-[myo-inositol-1-phosphoryl-6-D-mannopyranosyl-alpha1-2-myo-inositol-1-phosphate]; N-hexadecanoyl)-sphinganine-1-O-[myo-inositol-1-phosphoryl-6-D-mannopyranosyl-alpha1-2-myo-inositol-1-phosphate]; N-hexadecanoyl-1-beta-(3′-sulfo)-glucosyl-sphing-4-enine; N-octadecanoyl-1-beta-(3′-sulfo)-glucosyl-sphing-4-enine; Nonadecaphing-4-enine-1-phosphate; N-tetracosanoyl-1-beta-(3′-sulfo)-glucosyl-sphing-4-enine; Sphing-4-enine; Sphing-4-enine-1-phosphate; Sphing-4-enine-1-phosphocholine; Sphing-8Z-enine; sphinga-4E,14Z-dienine; sphinga-4E,8E,10E-trienine; sphinga-4E,8E-dienine; sphinga-4E,8Z-dienine; Sphinganine; Sphinganine-1-phosphate; Sphinganine-1-phosphocholine; tetradecasphing-4E-enine; and/or tetradecasphinga-4E,6E-dienine;

### Steroid

In some embodiments may be a steroid which includes a steroid nucleus, consisting of four fused rings, three cyclohexane rings and one cyclopentane ring.

In one embodiment, the sterol may be cholesterol. Cholesterol is amphipathic, with a polar head group (the hydroxyl group at C-3) and a nonpolar hydrocarbon body (the steroid nucleus and the hydrocarbon side chain at C-17), about as long as a 16-carbon fatty acid in its extended form. Cholesterol is a component of cellular membranes, myelin sheath, and brain and nerve tissue. It is also found in the liver and bile salts; large quantities of it are found in the skin, and some of it becomes vitamin D when the skin is exposed to direct sunlight. In the adrenal gland, cholesterol is used to synthesize steroid hormones.

In some embodiments, the lipid may be a derivative of cholesterol. Molecular geometry of cholesterol derivatives can affect delivery efficacy and biodistribution of lipoprotein systems. For example, cholesterol derivatives with C-24 alkyl phytosterols may increase the in vivo delivery efficacy of systems of the disclosure. The length of the hydrophobic tails of the cholesterol derivatives, the flexibility of sterol rings and the polarity of hydroxy groups may impact delivery efficacy.

In some embodiments, the cholesterol may be 24S-hydroxy-cholesterol (also herein 24(S)-hydroxycholesterol or cholest-5-en-3β,24S-diol).

Non limiting examples of cholesterol derivatives include (20R,22R)-20,22-dihydroxycholesterol (cholest-5-en-3beta,20R,22R-triol); (24E),26-hydroxydesmosterol (cholest-5,24E-dien-3beta,26-diol); (24E)-24,26-dimethyldesmosterol (24,26-dimethylcholesta-5,24E-dien-3beta-ol); (24R)-11alpha,20,24-trihydroxyecdysone ((22R,24R)-2beta,3beta,11alpha,14,20,22,24,25-octahydroxy-5beta-cholest-7-en-6-one); (24R)-24-hydroxycholesterol ((24R)-cholest-5-en-3beta, 24-diol); (24Z),26-hydroxydesmosterol (cholest-5,24Z-dien-3beta,26-diol); (24Z)-24,26-dimethyldesmosterol (24,26-dimethylcholesta-5,24Z-dien-3beta-ol); (25R)-11alpha,20,26-trihydroxyecdysone ((22R,25R)-2beta,3beta,11alpha,14,20,22,25,26-octahydroxy-5beta-cholest-7-en-6-one); (25S)-11alpha,20,26-trihydroxyecdysone ((22R,25S)-2beta,3beta,11alpha,14,20,22,25,26-octahydroxy-5beta-cholest-7-en-6-one); 1-(5alpha)-cholestenone ((5alpha)-cholest-1-en-3-one); 1,25-Dihydroxycholesterol (cholest-5-en-1alpha,3beta,25-triol); 1,4-cholestadienone (Cholesta-1,4-dien-3-one); 11-Hydroxy-cyasterone (2beta,3beta,11alpha-14,20,22R-hexahydroxy-26,28R-epoxy-5beta-stigmast-7-ene-6,26-dione); 12:0-Glc-Cholesterol (3-O-(6′-O-dodecanoyl-alpha-D-glucopyranosyl)-cholest-5-en-3beta-ol); 14beta-14,15-dihydrocalonysterone (2beta,3beta,6,20R,22R,25-hexahydroxy-14beta-cholesta-5,8(9)-dien-7-one); 14-demethyllanosterol (4,4-dimethyl-5alpha-cholesta-8,24-dien-3beta-ol); 14-Deoxy-20-hydroxyecdysone (2beta,3beta,20R,22R,25-pentahydroxy-5beta-cholesta-7-en-6-one); 14-deoxy-25-hydroxydacryhainansterone (2beta,3beta,20R,22R,25-pentahydroxy-cholesta-7,9(11)-dien-6-one); 14-deoxydacryhainansterone (2beta,3beta,20R,22R-tetrahydroxy-cholesta-7,9(11)-dien-6-one); 15alpha-hydroxycholestane (5alpha-cholestan-3beta,15alpha-diol); 15alpha-hydroxycholestene (5alpha-cholest-8(14)-en-3beta,15alpha-diol); 15beta-hydroxycholestane (5alpha-cholest-8(14)-an-3beta,15beta-diol); 15beta-hydroxycholestene (5alpha-cholest-8(14)-en-3beta,15beta-diol); 15-dehydro-A-norcholestanol (3beta-hydroxymethyl-A-nor-5alpha-cholest-15-ene); 15-ketocholestane (3beta-hydroxy-5alpha-cholestan-15-one); 15-ketocholestene (3beta-hydroxy-5alpha-cholest-8(14)-en-15-one); 16:0-Glc-Cholesterol (3-O-(6′-O-hexadecanoyl-beta-D-glucopyranosyl)-cholest-5-en-3beta-ol); 16:1-Glc-Cholesterol (3-O-(6′-O-(7Z-hexadecenoyl)-beta-D-glucopyranosyl)-cholest-5-en-3beta-ol); 16:2-Glc-Cholesterol (3-O-(6′-O-(7Z,10Z-hexadecadienoyl)-beta-D-glucopyranosyl)-cholest-5-en-3beta-ol); 16:3-Glc-Cholesterol (3-O-(6′-O-(7Z,10Z,13Z-hexadecatrienoyl)-beta-D-glucopyranosyl)-cholest-5-en-3beta-ol); 16beta,20S-dihydroxycholestan-3-one (16beta,20S-dihydroxy-5-alpha-cholestan-3-one); 17(20)-Dehydrocryptogenin (16,22-dioxo-25S-methyl-cholest-5,17(20)Z-dien-3beta-ol); 18:0-Glc-Cholesterol (3-O-(6′-O-octadecanoyl-beta-D-glucopyranosyl)-cholest-5-en-3beta-ol); 18:1-Glc-cholesterol (3-O-(6′-O-(9Z-octadecenoyl)-beta-D-glucopyranosyl)-cholest-5-en-3beta-ol); 18:2-Glc-cholesterol (3-O-(6′-O-(9Z,12Z-octadecadienoyl)-beta-D-glucopyranosyl)-cholest-5-en-3beta-ol); 18:2-Glc-Stigmasterol (3-O-(6′-O-(9Z,12Z-octadecadienoyl)-beta-D-glucopyranosyl)-stigmast-5,22E-dien-3beta-ol); 18:3-Glc-cholesterol (3-O-(6′-O-(9Z,12Z,15Z-octadecatrienoyl)-beta-D-glucopyranosyl)-cholest-5-en-3beta-ol); 19-hydroxycholesterol (Cholest-5-en-3beta,19-diol); 19-norcholestanol (19-nor-5alpha-cholestan-3beta-ol); 19-norcholestenone (19-norcholest-5-en-3-one); 2,22,25-trideoxyecdysone (3beta,14-dihydroxy-5beta-cholest-7-en-6-one); 20,26-dihydroxyecdysone (ergosta-5,24(28)-diene-3beta,20,26-triol); 20:0-Glc-Cholesterol (3-O-(6′-O-eicosanoyl-beta-D-glucopyranosyl)-cholest-5-en-3beta-ol); 20:1-Glc-cholesterol (3-O-(6′-O-(11Z-eicosenoyl)-beta-D-glucopyranosyl)-cholest-5-en-3beta-ol); 20:2-Glc-cholesterol (3-O-(6′-O-(11Z,14Z-eicosadienoyl)-beta-D-glucopyranosyl)-cholest-5-en-3beta-ol); 20:3-Glc-cholesterol (3-O-(6′-O-(11Z,14Z,17Z-eicosatrienoyl)-beta-D-glucopyranosyl)-cholest-5-en-3beta-ol); 20a-hydroxy cholesterol; 20-hydroxyecdysone (2beta,3beta,14alpha,20,22R,25-hexahydroxy-5beta-cholest-7-en-6-one); 20-hydroxyecdysone 22-phosphate ((22R)-2beta,3beta,14,20,25-pentahydroxy-6-oxo-5beta-cholest-7-en-22-yl dihydrogen phosphate); 20-oxodacryhainansterone (2beta,3beta,14alpha,20R-tetrahydroxy-cholesta-7,9(11)-dien-6,22-dione); 20S-Hydroxycholest-1-en-3,16-dione (20S-Hydroxy-5-alpha-cholest-1-en-3,16-dione); 20S-Hydroxycholest-4-en-3,16-dione (20S-Hydroxycholest-4-en-3,16-dione); 20S-Hydroxycholestane-3,16-dione (20S-Hydroxy-5-alpha-cholestane-3,16-dione); 20S-hydroxycholesterol (cholest-5-en-3beta,20S-diol); 22,23-epoxycampesterol (22,23-epoxycampest-5-en-3beta-ol); 22:0-Glc-Cholesterol (3-O-(6′-O-docosanoyl-beta-D-glucopyranosyl)-cholest-5-en-3beta-ol); 22:1-Glc-cholesterol (3-O-(6′-O-(13Z-docosenoyl)-beta-D-glucopyranosyl)-cholest-5-en-3beta-ol); 22:2-Glc-cholesterol (3-O-(6′-O-(13Z,16Z-docosadienoyl)-beta-D-glucopyranosyl)-cholest-5-en-3beta-ol); 22:3-Glc-cholesterol (3-O-(6′-O-(13Z,16Z,19Z-docosatrienoyl)-beta-D-glucopyranosyl)-cholest-5-en-3beta-ol); 22-dehydrocholesterol (cholesta-5,22E-dien-3beta-ol); 22-deoxy-20,21-dihydroxyecdysone (2beta,3beta,14,20,21,25-hexahydroxy-5beta-cholest-7-en-6-one); 22-hydroxycholesterol (cholest-5-en-3beta,22-diol); 22-iso-ecdysone (2beta,3beta,14alpha,22S,25-pentahydroxy-5beta-cholest-7-en-6-one); 22-methylenecholesterol (23-methylene-cholest-5-en-3beta-ol); 22-oxocholesterol (22-oxo-cholest-5-en-3beta-ol); 22R-hydroxycholesterol (cholest-5-en-3beta,22R-diol); 22R-hydroxycholesterol(d7) (cholest-5-en-3beta,22R-diol(d7)); 22S-hydroxycholesterol (cholest-5-en-3beta,22S-diol); 22S-hydroxycholesterol(d7) (cholest-5-en-3beta,22S-diol(d7)); 22Z-dehydrocholesterol (cholesta-5,22Z-dien-3beta-ol); 24,25-dihydrolanosterol (5alpha-lanost-8-en-3beta-ol); 24,25-epoxy-cholesterol (24S,25-epoxy-cholest-5-en-3beta-ol); 24,25-epoxy-cholesterol(d6) (24,25-epoxy-cholest-5-en-3beta-ol(d6)); 24alpha-Methoxydihydrocucurbitacin D; 24beta-Methoxydihydrocucurbitacin D; 24-hydroxy-cholesterol(d6) (cholest-5-en-3beta,24-diol(d6)); 24-keto-25dehydrocholestanol (24-oxocholest-25-en-3beta-ol); 24-keto-25dehydrocholesterol (24-oxocholesta-5,25-dien-3beta-ol); 24-ketocholestanol (24-oxo-5alpha-cholestan-3beta-ol); 24-norcholesterol (24-nor-cholest-5-en-3beta-ol); 24-northornasterol A (24-nor-3beta,6alpha,20S-trihydroxy-5alpha-cholest-9(11)-en-23-one); 24-oxocholesterol (24-oxo-cholest-5-en-3beta-ol); 24S,25-dihydroxycholesterol (cholest-5-en-3beta,24S,25-triol); 24S,27-dihydroxycholesterol (cholest-5-en-3beta,24S,26-triol); 24S-hydroxy-cholesterol (cholest-5-en-3beta,24S-diol); 24S-hydroxycholesterol 3-sulfate, 24-D-glucuronide ((24S)-cholest-5-en-3beta,24-diol 3-sulfate 24-D-glucuronide); 24S-OH-7-DHC (cholesta-5,7-dien-3beta,24S-diol); 25,27-dihydroxycholesterol (cholest-5-en-3beta,25,26-triol); 25-Azacholesterol; 25-deoxyecdysone ((22R)-2beta,3beta,14,22-tetrahydroxy-5beta-cholest-7-en-6-one); 25-Hydroxy-7-oxocholesterol (3beta,25-Dihydroxycholest-5-en-7-one); 25-Hydroxy-atrotosterone A (2beta,3beta,11alpha,14alpha,20,22R,25-heptahydroxy-24R-methyl-5beta-cholest-7-en-6-one); 25-hydroxy-cholesterol (cholest-5-en-3beta,25-diol); 25-hydroxy-cholesterol 3-sulfate (Cholest-5-en-3beta,25-diol 3-sulfate); 25-hydroxy-cholesterol(d3) (cholest-5-en-3beta,25-diol(d3)); 25-hydroxy-cholesterol(d6) (cholest-5-en-3beta,25-diol(d6)); 26,27-bisnor-22-dehydro-cholesterol (26,27-bisnor-cholesta-5,22E-dien-3beta-ol); 27-hydroxy-cholesterol (cholest-5-en-3beta,26-diol); 27-hydroxy-cholesterol(25R) (cholest-(25R)-5-en-3beta,26-diol); 27-hydroxy-cholesterol(d6) (cholest-5-en-3beta,27-diol(d6)); 27-Nor-cholestane-pentol (27-Nor-5b-cholestane-3a,7a,12a,24,25-pentol); 2-dehydroecdysone ((22R)-3beta,14,22,25-tetrahydroxy-5beta-cholest-7-ene-2,6-dione); 2-deoxy-20-hydroxy-5alpha-ecdysone 3-acetate ((22R)-14,20,22,25-tetrahydroxy-6-oxo-5alpha-cholest-7-en-3beta-yl acetate); 2-deoxy-20-hydroxyecdysone 22-phosphate ((22R)-3beta,14,20,25-tetrahydroxy-6-oxo-5beta-cholest-7-en-22-yl dihydrogen phosphate); 2-deoxyecdysone ((22R)-3beta,14,22,25-tetrahydroxy-5beta-cholest-7-en-6-one); 2-deoxyecdysone 22-phosphate ((22R)-3beta,14,25-trihydroxy-6-oxo-5beta-cholest-7-en-22-yl dihydrogen phosphate); 2-Oxo-turkesterone (22-oxo-2beta,3beta,11alpha,14alpha,20,25-hexahydroxy-5beta-cholest-7-en-6-one); 3,5-cholestadien-7-one (cholesta-3,5-dien-7-one); 32-hydroxylanosterol (4,4-dimethyl-14alpha-hydroxymethyl-5alpha-cholesta-8,24-dien-3beta-ol); 32-oxolanosterol (4,4-dimethyl-14alpha-formyl-5alpha-cholesta-8,24-dien-3beta-ol); 3beta,4beta-dihydroxycholest-23-one (3beta,4beta-dihydroxycholest-23-one); 3beta,4beta-dihydroxycholest-7(8)-en-23-one (3beta,4beta-dihydroxycholest-7(8)-en-23-one); 3beta,4beta-dihydroxycholest-8(9)-en-23-one (3beta,4beta-dihydroxycholest-8(9)-en-23-one); 3beta,4beta-dihydroxycholesta-5-ene-23-one (3beta,4beta-dihydroxycholest-5(6)-en-23-one); 3beta-hydroxy-4alpha-methyl-5alpha-cholest-7-ene-4beta-carboxylic acid (3beta-hydroxy-4alpha-methyl-5alpha-cholest-7-ene-4beta-carboxylic acid); 3beta-hydroxy-4beta-methyl-5alpha-cholest-7-ene-4alpha-carbaldehyde (3beta-hydroxy-4beta-methyl-5alpha-cholest-7-ene-4alpha-carbaldehyde); 3beta-hydroxy-4beta-methyl-5alpha-cholest-7-ene-4alpha-carboxylic acid (3beta-hydroxy-4beta-methyl-5alpha-cholest-7-ene-4alpha-carboxylic acid); 3beta-Hydroxy-5alpha,6alpha-epoxy-9-oxo-9,10-seco-5-cholest-7-en-11-al (3beta-hydroxy-5alpha,6alpha-epoxy-9-oxo-9,10-seco-5-cholest-7-en-11-al); 3beta-Hydroxy-7-oxochol-5-en-24-oyl-CoA (3beta-Hydroxy-7-oxochol-5-en-24-oyl-CoA); 3-dehydro-4-methylzymosterol (4-methyl-5alpha-cholesta-8,24-dien-3-one); 3-dehydroecdysone ((22R)-2beta,14,22,25-tetrahydroxy-5beta-cholest-7-ene-3,6-dione); 3-keto-4alpha-methyl-zymosterol (4alpha-methyl-5alpha-cholesta-8,24-dien-3-one); 3-ketocholesterol (Cholest-5-en-3-one); 3-O-acetylecdysone 2-phosphate ((22R)-14,22,25-trihydroxy-6-oxo-2beta-(phosphonooxy)-5beta-cholest-7-en-3beta-yl acetate); 3-oxo-7alpha-hydroxycholesterol (7alpha-hydroxy-cholest-5-en-3-one); 4,4-dimethyl-5alpha-cholest-7-en-3beta-ol (4,4-dimethyl-5alpha-cholest-7-en-3beta-ol); 4,4-Dimethylcholest-8-enol (4,4-Dimethyl-5alpha-cholesta-8-en-3beta-ol); 4,4-dimethylcholesta-8,11,24-trienol (4,4-dimethyl-5alpha-cholesta-8,14,24-trien-3beta-ol); 4,4-dimethylcholesta-8,11-dienol (4,4-dimethyl-5alpha-cholesta-8,14-dien-3beta-ol); 4,6-cholestadienol (Cholesta-4,6-dien-3beta-ol); 4,6-cholestadienone (cholesta-4,6-dien-3-one); 4alpha-carboxy-zymosterol (4alpha-carboxy-5alpha-cholesta-8,24-dien-3beta-ol); 4alpha-methylcholest-8-en-3beta-ol (4alpha-methyl-5alpha-cholest-8-en-3beta-ol); 4alpha-methylzymosterol (4alpha-methyl-5alpha-cholesta-8,24-dien-3beta-ol); 4alpha-OH-7-DHC (cholesta-5,7-dien-3beta,4alpha-diol); 4beta-(hydroxymethyl)-4alpha-methyl-5alpha-cholest-7-en-3beta-ol (4beta-(hydroxymethyl)-4-methyl-5alpha-cholest-7-en-3beta-ol 4beta-(hydroxymethyl)-4alpha-methyl-5alpha-cholest-7-en-3beta-ol); 4beta-hydroxy-cholesterol (cholest-5-en-3beta,4beta-diol); 4beta-hydroxy-cholesterol(d7) (cholest-5-en-3beta,4beta-diol(d7)); 4beta-methyl-zymosterol-4alpha-carboxylic acid (3beta-hydroxy-4-methyl-5alpha-cholesta-8,24-diene-4alpha-carboxylic acid); 4beta-OH-7-DHC (cholesta-5,7-dien-3beta,4beta-diol); 4-cholestenol (Cholest-4-en-3beta-ol); 5, 20(22)-cholestadienol (Cholesta-5, 20(22)E-dien-3beta-ol); 5,6alpha-epoxy-cholesterol (5alpha,6alpha-epoxy-5alpha-cholestan-3beta-ol); 5,6alpha-epoxy-cholesterol(d7) (5,6alpha-epoxy-5alpha-cholestan-3beta-ol(d7)); 5,6beta-epoxy-cholesterol(d7) (5,6beta-epoxy-5beta-cholestan-3beta-ol(d7)); 5alpha,14beta-14-deoxy-20-hydroxyecdysone (2beta,3beta,20R,22R,25-pentahydroxy-5beta,14beta-cholesta-7-en-6-one); 5alpha,6alpha-24S,25-diepoxy-cholesterol (5alpha,6alpha-24S,25-diepoxy-cholestan-3beta-ol); 5alpha,6beta-dihydroxycholestanol (cholestane-3beta,5alpha,6beta-triol); 5alpha-14-Deoxy-20-hydroxyecdysone (2beta,3beta,20R,22R,25-pentahydroxy-5alpha-cholesta-7-en-6-one); 5alpha-cholesta-7,24-dien-3beta-ol (5alpha-cholesta-7,24-dien-3beta-ol); 5alpha-cholestanone (5alpha-cholestan-3-one); 5alpha-hydroxycholesterol (Cholestan-3beta,5alpha-diol); 5beta,6beta-epoxy-cholesterol (5beta,6beta-epoxy-cholestan-3beta-ol); 5beta-cholestanone (5beta-cholestan-3-one); 5-deoxykaladasterone (2beta,3beta,14alpha,20R,22R-pentahydroxy-cholesta-7,9(11)-dien-6-one); 6,24S-dihydroxycholesterol (cholest-5-en-3beta,6,24S-triol); 6alpha-hydroxycholestanol (cholestan-3beta,6alpha-diol); 6alpha-hydroxycholestanol(d7) (cholestan-3beta,6alpha-diol(d7)); 6-oxocholestanol (5alpha-cholestan-6-oxo-3beta-ol); 7alpha,(25R)26-Dihydroxycholest-4-en-3-one ((25R)-7alpha,26-Dihydroxycholest-4-en-3-one); 7alpha,(25S)26-Dihydroxycholest-4-en-3-one ((25S)-7alpha,26-Dihydroxycholest-4-en-3-one); 7alpha,12alpha,(25R)26-Trihydroxycholest-4-en-3-one ((25R)-7alpha,12alpha,26-trihydroxycholest-4-en-3-one); 7alpha,25(R)26-dihydroxycholesterol ((25R)-Cholest-5-en-3beta,7alpha,26-triol); 7alpha,25(S)26-dihydroxycholesterol ((25S)-Cholest-5-en-3beta,7alpha,26-triol); 7alpha-hydroperoxycholesterol (7alpha-peroxycholest-5-en-3beta-ol); 7alpha-hydroxy-cholesterol (cholest-5-en-3beta,7alpha-diol); 7alpha-hydroxy-cholesterol(d7) (cholest-5-en-3beta,7alpha-diol(d7)); 7alpha-hydroxy-Desmosterol (cholest-5,24-dien-3beta,7alpha-diol); 7beta,25-dihydroxycholesterol (Cholest-5-ene-3beta,7beta,25-triol); 7beta-hydroperoxycholesterol (7beta-peroxycholest-5-en-3beta-ol); 7beta-Hydroxy-3-epi-isocucurbitacin B; 7beta-hydroxycholesterol (5-cholestene-3beta,7beta-diol); 7beta-hydroxy-cholesterol(d7) (cholest-5-en-3beta,7beta-diol(d7)); 7-dehydrocholesterol (cholesta-5,7-dien-3beta-ol); 7-dehydrocholesterol-d7 (Cholesta-5,7-dien-3beta-ol-d7); 7-dehydro-desmosterol (cholest-5,7,24-trien-3beta-ol); 7-OOH-5,8-dien-3beta-ol (7alpha-hydroperoxy-cholesta-5,8-dien-3beta-ol); 7-oxo-cholestenone (Cholest-5-en-3,7-dione); 7-oxo-cholesterol (7-oxo-cholest-5-en-3beta-ol); 7-oxocholesterol(d7) (7-oxo-cholest-5-en-3beta-ol(d7)); 8-Dehydrocholesterol (cholesta-5,8-dien-3beta-ol); Acetylpinnasterol (2beta-acetoxy-3alpha,20R,22S-trihydroxy-cholesta-4,7-dien-6-one); Arvenin I; Arvenin II; Arvenin III; Arvenin IV; Bombycosterol (Cholesta-7,14-diene-3beta,5alpha,6alpha,20,25-pentaol); Brainesteroside A (3-O-(beta-D-glucopyranosyl)-2beta,3beta,20,22R-tetrahydroxy-14alpha,15alpha-epoxy-5beta-cholest-7-en-6-one); Brainesteroside B (3-O-(beta-D-glucopyranosyl)-2beta,3beta,20,22R-tetrahydroxy-5beta-cholest-7,14-dien-6-one); Brainesteroside C (3-O-(beta-D-glucopyranosyl)-2beta,3beta,6,20,22R-pentahydroxy-cholest-5,8,14-trien-7-one); Brainesteroside D (3-O-(beta-D-glucopyranosyl)-2beta,3beta,14alpha,22R-tetrahydroxy-5beta-cholest-7-ene-6-one); Brainesteroside E (3-O-(beta-D-glucopyranosyl)-2beta,3beta,14alpha,20R,22R-pentahydroxy-5alpha-cholest-7-ene-6-one); Calibagenin (cholest-5-en-3beta,16beta,22S-triol); Calonysterone (2beta,3beta,6,20R,25-hexahydroxycholesta-5,8(9),14-trien-7-one); carthamosterone; Certonardosterol F (5alpha-cholestan-3beta,6alpha,15betaa,24S-tetrol); Certonardosterol G (5alpha-cholest-22E-en-3beta,6alpha,15beta,24R-tetrol); Cholesta-5,7-diene-1,3-diol (cholesta-5,7-diene-1alpha,3beta-diol); cholestan-6-oxo-3,5-diol (cholestan-6-oxo-3beta,5alpha-diol); Cholestane skeleton; Cholestane-3b,5a,6b,(25R)26-tetrol (Cholestane-3b,5a,6b,(25R)26-tetrol); Cholestanyl glucoside (3-O-(beta-D-glucopyranosyl)-5alpha-cholestan-3beta-ol); Cholestenone (cholest-4-en-3-one); Cholesterol (cholest-5-en-3beta-ol); Cholesterol glucuronide; Cholesterol(d7) (cholest-5-en-3beta-ol(d7)); Cholesterol-5alpha-hydroperoxide (3beta-hydroxy-5alpha-cholest-6-ene-5-hydroperoxide); Cholesterol-5beta-hydroperoxide (5beta-hydroperoxy-cholest-6-ene-3beta-ol); Cholesterol-6alpha-hydroperoxide (3beta-hydroxycholest-4-ene-6alpha-hydroperoxide); Cholesterol-6beta-hydroperoxide (3beta-hydroxycholest-4-ene-6beta-hydroperoxide); Cholesteryl 6-O-palmitoyl-beta-D-galactopyranoside (3-O-(6′-O-hexadecanoyl-beta-D-galactopyranosyl)-cholest-5-en-3beta-ol); Cholesteryl acetate (3beta-acetoxycholest-5-ene); cholesteryl beta-D-galactoside (cholest-5-en-3beta-yl beta-D-galactopyranoside); cholesteryl beta-D-glucoside (cholest-5-en-3beta-yl beta-D-glucopyranoside); cholesteryl beta-D-xyloside (cholest-5-en-3beta-yl beta-D-xylopyranoside); Colocynthenin E; Colocynthenin F; Common Name Coprostanol (5beta-cholestan-3beta-ol), Cryptogenin (16,22-dioxo-25S-methyl-cholest-5-en-3beta-ol), Cucurbitacin A, Cucurbitacin B, Cucurbitacin C, Cucurbitacin D, Cucurbitacin E, Cucurbitacin F, Cucurbitacin H, Cucurbitacin I, Cucurbitacin J, Cucurbitacin L, Cucurbitacin 0, Cucurbitacin P, Cucurbitacin Q, Cucurbitacin S, Cyathsterone A ((22R,25R,28R,32R)-2beta,3beta,14alpha,20R-33-pentahydroxy-25-ethyl-28beta-(1-hydroxybutan-2-yl)-6-oxo-5beta-cholest-7-ene-27,29-dioxocan), Cyathsterone D ((20R,22R,23R,25R)-2beta,3beta,14alpha,20,22-26-hexahydroxy-6-oxo-23-methyl-cholest-7-ene), Cystosterol ((20,23-cyclo)-cholest-5-en-3beta-ol), Desmosterol (cholest-5,24-dien-3beta-ol), Desmosterol(d6) (cholest-5,24-dien-3beta-ol(d6)), DHCEO (3beta,5alpha-dihydroxycholest-7-en-6-one), Dihydrocholesterol (cholestan-3beta-ol), Dihydromarthasterone (3beta,6alpha-dihydroxy-5alpha-cholesta-9(11)-en-23-one), Dormantinol (cholest-5-en-3beta,22S,26S-triol), Dormatinone ((25S)-3beta,26-dihydroxycholest-5-en-22-one), Ecdysone (2beta,3beta,14alpha,22R,25-pentahydroxy-5beta-cholest-7-en-6-one), Ecdysone 22-linoleate (22-O-(9Z,12Z-octadecdienoyl)-2beta,3beta,14alpha,22R,25-pentahydroxy-5beta-cholest-7-en-6-one), Ecdysone 22-oleate (22-O-(9Z-octadecenoyl)-2beta,3beta,14alpha,22R,25-pentahydroxy-5beta-cholest-7-en-6-one), Ecdysone 22-palmitate (22-O-hexadecanoyl-2beta,3beta,14alpha,22R,25-pentahydroxy-5beta-cholest-7-en-6-one), Ecdysone 22-palmitoleate (22-O-(9Z-hexadecenoyl)-2beta,3beta,14alpha,22R,25-pentahydroxy-5beta-cholest-7-en-6-one), Ecdysone 22-stearate (22-O-octadecanoyl-2beta,3beta,14alpha,22R,25-pentahydroxy-5beta-cholest-7-en-6-one), ecdysone 25-O-D-glucopyranoside ((22R)-2beta,3beta,14,22-tetrahydroxy-6-oxo-5beta-cholest-7-en-25-yl D-glucopyranoside), Echissaposterol (27-nor-23,26,26-trimethyl-cholest-5-en-3beta-ol), Ehrensteroid B (11alpha-acetoxy-23-methylcholesta-22(E)-en-3beta,5alpha,6beta-triol), Endertiin A (3,11-dioxo-5alpha-lanosta-8,24E-dien-26-al), Endertiin B (3beta,26-dihydroxy-5alpha-lanosta-11-oxo-8,24E-diene), EnP(5,8) (5alpha,8alpha-epidioxy-cholest-6-en-3beta-ol), Epi-cholestanol (5alpha-cholestan-3alpha-ol), Epicholesterol (cholest-5-en-3alpha-ol), Fasciculic acid B (Lanost-8-ene-2alpha,3beta,12alpha,24R,25-pentol, 2-(3S-hydroxy-3-methylpentanedioate)), Fasciculic acid C (Lanost-8-ene-2alpha,3beta,12alpha,21,24R,25-hexol, 3-(N-Glycyl-3S-hydroxy-3-methylpentanedioate)), Fibraurecdyside A (3-O-(beta-D-glucopyranosyl)-2beta,3beta,14alpha,20,22R,25-hexahydroxy-24R-methyl-5beta-cholest-7-en-6-one), Ganodecalone A (26-hydroxy-5alpha-lanosta-3,11-dioxo-8,24E-diene), Ganodecalone B (27-nor-lanosta-8,23E-dien-3,11,25-trione), Ganoderic acid A ((25R)-3,11,23-trioxo-7beta,15alpha-dihydroxy-lanost-8-ene-26-oic acid), Ganoderic acid B ((25R)-11,15,23-trioxo-3beta,7beta-dihydroxy-lanost-8-ene-26-oic acid), Ganosporeric acid A ((25R)-3,7,11,12,15,23-hexaoxo-lanost-8-ene-26-oic acid), Ganoweberianic acid A (25S-15beta-hydroxy-3,7,11,23-tetraoxolanosta-8-en-26-oic acid.), Ganoweberianic acid B (25S-3alpha,15beta-dihydroxy-7,11,23-trioxolanosta-8-en-26-oic acid.), Ganoweberianic acid C (25S-3alpha,12beta,15beta-trihydroxy-7,11,23-trioxolanosta-8-en-26-oic acid.), Ganoweberianic acid D (25S-12beta,15beta-dihydroxy-3,7,11,23-tetraoxolanosta-8-en-26-oic acid.), Ganoweberianic acid E (25S-12beta-acetoxy-15beta-hydroxy-3,7,11,23-tetraoxolanosta-8-en-26-oic acid.), Ganoweberianic acid F (25S-15beta-hydroxy-3,7,11,12,23-pentaoxolanosta-8-en-26-oic acid.), Ganoweberianic acid G (25S-3beta,15beta,28-trihydroxy-7,11,23-trioxolanosta-8-en-26-oic acid.), Ganoweberianone A (26-(3′-oxo-lanosta-7′,9′(11′)-dien-24'S,25′R,26′-triol)-25S-15beta-hydroxy-3,7,11,23-tetraoxolanosta-8-en-ate), Ganoweberianone B (26-(3′-oxo-lanosta-7′,9′(11′)-dien-24'S,25′R,26′-triol)-25S-3alpha,15beta-dihydroxy,7,11,23-trioxolanosta-8-en-ate), Herbasterol (9-oxo-9,11-seco-5beta-cholestan-2beta,3alpha,6beta,11,19-pentol), Hexanorisocucurbitacin D, Hipposterol (5,6-seco-cholest-7-en-3beta,5beta,6-triol), inokosterone ((22R,25R)-2beta,3beta,14,20,22,26-hexahydroxy-5beta-cholest-7-en-6-one), lanostane (lanostane), Lanosterol (lanosta-8,24-dien-3beta-ol), Lathosterol (cholest-7-en-3beta-ol), Lesterone (2alpha,3alpha,11beta,14alpha,20R,22R,25-heptahydroxy-5beta-cholest-7-en-6-one), Lobophysterol A (25-hydroperoxy,11 alpha-acetoxy-cholest-23E-en-3beta,5alpha,6beta-triol), Lophanol (4alpha-methyl-5alpha-cholestan-3beta-ol), Lophenol (4alpha-methyl-cholest-7-en-3beta-ol), Lophenone (4alpha-methyl-5alpha-cholest-7-en-3-one), Lumisterol 3, Marthasterone (3beta,6alpha-dihydroxy-5alpha-cholesta-9(11),24-dien-23-one), Masticadienonic acid (3-Oxolanosta-7,24Z-dien-26-oic acid), Menellsteroid E (1beta,3beta,5alpha,11 beta-tetrahydroxycholestan-6-one), Menellsteroid F (Cholest-24-ene-3beta,5alpha,6beta,11 beta-tetraol), Menellsteroid H (24-nor-cholest-22(E)-ene-3beta,5\hat{l}=alpha,6beta,11beta-tetraol), Micaceol (4-methylidene-cholest-5-en-3beta-ol), Nebrosteroid J (Methyl 3beta-hydroxy-cholest-5,22E-dien-19-oate), Nebrosteroid K (Methyl 3beta-hydroxy-cholest-5-en-19-oate), Neocucurbitacin E, Patinosterol ((24S)-26-nor-5alpha-cholest-22E-en-3beta-ol), Penasterol (4,4-dimethyl-14-carboxy-cholesta-9(10),24-dien-3beta-ol), Pentalinonside (14,16-14,21-15,20-triepoxy-14,15-secopregnan-5-en-3-ol 3-O-beta-d-diginopyranoside), Pentalinonsterol (Cholest-4,20,20-trien-3-one), Pinnasterol (2beta,3alpha,20R,22S-tetrahydroxy-cholesta-4,7-dien-6-one), polypodine B ((2beta,3beta,5beta,22R)-2,3,5,14,20,22,25-heptahydroxycholest-7-en-6-one), ponasterone A ((22R)-2beta,3beta,14,20,22-pentahydroxy-5beta-cholest-7-en-6-one), Saxosterol (cholest-5-en-3beta,16beta,22R-triol), Secosterol-A (3beta,5-oxo-5,6-secocholestan-6-al), Secosterol-B (3beta,5beta-dihydroxy-b-norcholestan-6beta-carboxaldehyde), shidasterone (2beta,3beta,14,20-tetrahydroxy-22,25-epoxy-5beta-cholest-7-en-6-one), Spongioside A (3-O-(Rhaa1-4(Rhaa1-2)Glcb)-16-O-(Glcb)-cholest-5-en-3beta,16beta,22S-triol), Spongioside B (3-O-(Rhaa1-4(Rhaa1-2)Glcb)-16-O-(6-O-acetyl-Glcb)-12-oxo-cholest-5-en-3beta,16beta,22S-triol), Subergosterone A (9alpha-hydroxy-5alpha-cholest-1,22E-dien-3-one), Suberoretisteroid A ((3S,16S,20R,22R,24S)-16,24:20,24-diepoxychol-est-5-ene-3,22,25-triol 3-acetate), Suberoretisteroid B ((3S,16S,20R,22R,24S)-16,24:20,24-diepoxycholest-5-ene-3,22,25-triol 22-acetate), Suberoretisteroid C ((3S,16S,20R,22R,24S)-16,24:20,24-diepoxycholest-5-ene-3,22,25-triol 3,22-diacetate), Suberoretisteroid D ((3S,7S,16S,20R,22R,24S)-16,24:20,24-diepoxycholest-5-ene-3,7,22,25-tetrol 22-acetate), Suberoretisteroid E ((3S,5S,16S,20R,22R,24S)-16,24:20,24-diepoxycholest-6-ene-3,5,22,25-tetrol 22-acetate), Sycosterol A (3alpha,7alpha,8beta,15beta,16alpha-pentahydroxy-5I±-cholestane-26-sulfate), Thornasterol A (3beta,6alpha,20S-trihydroxy-5alpha-cholest-9(11)-en-23-one), Trihydroxycoprostanoic acid (4beta-carboxy-5alpha-cholestane-3alpha,7alpha,12alpha-triol), Turkesterone (2beta,3beta,11alpha,14alpha,20,22R,25-heptahydroxy-5beta-cholest-7-en-6-one), Turkesterone 22-acetate (22beta-acetoxy-2beta,3beta,11alpha,14alpha,20,25-hexahydroxy-5beta-cholest-7-en-6-one), Turkesterone 22-acetonide (20,22beta-O-isopropylidene-2beta,3beta,11alpha,14alpha,20,22R,25-heptahydroxy-5beta-cholest-7-en-6-one), Venustone (22-O-[(3R)-3-hydroxybutanoyl]-2beta,3beta,14alpha,20,22R,25-hexahydroxy-5beta-cholest-7-en-6-one), Verrucorosteroid A ((3S,5S,16S,20R,22R,24S)-16,24:20,24-diepoxycholest-6-ene-3,5,22,25-tetrol 3,22-diacetate), Verrucorosteroid B ((3S,7S,16S,20R,22R,24S)-16,24:20,24-diepoxycholest-5-ene-3,7,22,25-tetraol), Verrucorosteroid C ((3S,16S,20R,24S)-16,24:20,24-diepoxycholest-5-ene-3,25-diol), Verrucorosteroid F (20S-24S-epoxy-cholest-5-en-3beta,22S-diol), Verrucorosterone (3beta-acetoxy-22S,25-trihydroxy-cholest-5,22E-dien-24-one), Ximaosteroid F (20R,22R-dihydroxy-5alpha-campesta-1,4-dien-3-one), Zoanthone A (25-acetoxy-2beta,3beta,11alpha,14alpha,20R,22R-hexahydroxy-5beta-cholest-7-en-6-one), Zoanthusterone (1beta,2beta,3beta,14alpha,20R,22R-hexahydroxy-5beta-cholest-7-en-6-one), Zymostenol (5alpha-cholest-8-en-3beta-ol), Zymostenol-d7 (5alpha-cholest-8-en-3beta-ol-d7), Zymosterol (5alpha-cholesta-8,24-dien-3beta-ol), zymosterol intermediate 1a (4-(hydroxymethyl)-5alpha-cholesta-8,24-dien-3beta-ol), zymosterol intermediate 1b ((20R)-3beta-hydroxy-5alpha-cholesta-8,24-diene-4-carbaldehyde), zymosterol intermediate 1c (1-(3beta-hydroxy-5alpha-cholesta-8,24-dien-4-yl)ethanone), Zymosterone (5alpha-cholesta-8,24-dien-3-one), (22,23-dinor)-24-vinyl-cholest-5-en-3beta,24-diol, (25S)-5alpha-cholestan-3beta,4beta,6alpha,7alpha,8beta,15beta,16beta,26-octol, (25S)-5alpha-cholestan-3beta,4beta,6alpha,7beta,8beta, 15alpha,16beta,26-octol, (25S)-5alpha-cholestan-3beta,4beta,6alpha,8beta,15alpha,16beta,26-heptol, (25S)-5alpha-cholestan-3beta,6alpha,7beta,8beta,15alpha,16beta,26-heptol, (25S)-5alpha-cholestan-3beta,6alpha,8,15beta,16beta,26-hexol, (25S)-5alpha-cholestan-3beta,6alpha,8beta,15alpha,16beat,26-hexol, (25S)-5alpha-cholestan-3beta,6beta,15alpha,16beta,26-pentol, 11-acetoxy-3beta,6alpha-dihydroxy-9,11-seco-5alpha-cholest-7-en-9-one., 12beta,16beta,20R-trihydroxy-cholest-1,4-dien-3-one, 12beta-hydroxy-24-norcholesta-1,4,22E-trien-3-one, 14alpha-methyl-5alpha-cholest-9(11)-en-3beta-ol, 14-Methyl-cholest-8-en-3beta-ol, 15,20-dihydroxycholest-4,14-dien-3,16-dione, 23R-methoxycholest-5,24-dien-3beta-ol, 24-vinyloxy-cholest-5,23Z-dien-3beta-ol, 26,27-dinor-cholest-5-en-23-yn-3beta-ol, 27-nor-22R-hydroxy-5alpha-cholestan-3,6-dione, 2alpha,7beta,15beta,18-tetraacetoxy-cholest-5-en-3alpha-ol, 3a,7a,12a-Trihydroxy-5b-24-oxocholestanoyl-CoA, 3a,7a,12a-Trihydroxy-5b-cholest-24-enoyl-CoA, 3a,7a-Dihydroxy-5b-24-oxocholestanoyl-CoA, 3a,7a-Dihydroxy-5b-cholest-24-enoyl-CoA, 3a,7a-Dihydroxy-5b-cholestanoyl-CoA, 3alpha,7alpha,12alpha-Trihydroxy-5beta-cholestanoyl-CoA, 3beta,5alpha,9alpha-trihydroxycholest-7-en-6-one, 3beta,5alpha,9alpha-trihydroxycholesta-7,14-dien-6-one, 3beta,5alpha-dihydroxycholesta-7,9(11)-dien-6-one, 3beta-acetoxy-cholest-5-en-7-one, 3beta-hydroxy-24-norchol-5-en-23-al, 3beta-hydroxy-26,27-bisnorcholest-5-en-24-one, 3beta-hydroxy-5alpha-cholest-8(9),14(15)-dien-23-one, 3beta-methoxycholest-5-ene, 4,14-Dimethylcholest-8,24-dien-3beta-ol, 4,4,14alpha-trimethyl-5alpha-cholest-9(11),24-dien-3beta-ol, 4,4-dimethyl-14alpha-formyl-5alpha-cholest-8-en-3beta-ol, 4,4-dimethyl-14alpha-hydroxymethyl-5alpha-cholest-8-en-3beta-ol, 4alpha,14alpha-dimethyl-5alpha-cholest-9(11)-en-3beta-ol, 4alpha-carboxy-4beta-methyl-5alpha-cholesta-8-en-3beta-ol, 4alpha-carboxy-5alpha-cholesta-8-en-3beta-ol, 4alpha-formyl-4beta-methyl-5alpha-cholesta-8,24-dien-3beta-ol, 4alpha-formyl-4beta-methyl-5alpha-cholesta-8-en-3beta-ol, 4alpha-Formyl-5alpha-cholesta-8,24-dien-3beta-ol, 4alpha-formyl-5alpha-cholesta-8-en-3beta-ol, 4alpha-hydroxymethyl-4beta-methyl-5alpha-cholesta-8,24-dien-3beta-ol, 4alpha-hydroxymethyl-4beta-methyl-5alpha-cholesta-8-en-3beta-ol, 4alpha-hydroxymethyl-5alpha-cholesta-8,24-dien-3beta-ol, 4alpha-hydroxymethyl-5alpha-cholesta-8-en-3beta-ol, 4alpha-methyl-5alpha-cholesta-8-en-3-one, 4alpha-methyl-5beta-cholestan-3beta-ol, 4alpha-methyl-cholest-5-en-3beta-ol, 4-Methyl-cholest-5-en-3beta-ol, 5,6alpha-epoxy-cholest-7-en-3beta,9alpha-diol, 5,6alpha-epoxy-cholest-8(14)-en-3beta,7alpha-diol, 5,6alpha-epoxy-cholest-8(14)-en-3beta,7beta-diol, 5a-Cholestane-3a,7a,12a,23,25-pentol, 5alpha,9alpha-epidioxy-8,14alpha-epoxy-cholest-6-en-3beta-ol, 5alpha,9alpha-epidioxy-cholest-7-en-3beta,6alpha-diol, 5alpha,9alpha-epidioxy-cholest-7-en-3beta,6beta-diol, 5alpha,9alpha-epidioxy-cholesta-6,8(14)-dien-3beta-ol, 5alpha-cholest-22E-en-3beta-ol, 5alpha-cholest-8-en-3-one, 5alpha-cholesta-24-en-3beta,20beta-diol-23-one, 5alpha-cholesta-7,24-dien-3beta-ol-d6, 5alpha-cholesta-9(11)-en-3beta, 20beta-diol, 5alpha-cholestan-3alpha,12alpha,16alpha-triol, 5alpha-cholestan-3beta,6alpha,8beta,15alpha,24S-triol, 5b-Cholestane-3a,7a,12a,23R,25-pentol, 5b-Cholestane-3a,7a,12a,23S,25-pentol, 5beta-cholest-22E-en-3beta-ol, 5beta-cholestan-3alpha,4alpha,11beta,12beta,21-pentol-3,21-disulfate, 5beta-cholestane, 7alpha-hydroxy-4-cholesten-3-one-d7, 9,11alpha-epoxy-6alpha-acetoxy-cholest-7-en-3beta,5alpha,19-triol, 9alpha,11alpha-epoxycholest-7-en-3beta,5alpha,6beta-triol, cholest-5,24-dien-3beta-yl beta-D-glucopyranoside, cholest-5-en-23-yn-3beta-ol, Cholest-5-ene, cholest-7-en-3beta,5alpha,6alpha,9alpha-tetrol, Cholest-7-en-3beta,5alpha,6beta,9alpha-tetrol, cholest-8(14)-en-3beta-ol, cholesta-4,6,8(14)-trien-3beta-ol, cholesta-5,7,8(14),22E-tetraen-3-one, cholesta-6,8(14)-dien-3beta,5alpha-diol, Lanost-9(11)-3beta-ol, and/or Lanosta-7,9(1)-dien-3eta-ol.

### Sterol Ester

In some embodiments, the lipid of the disclosure may be an ester of a sterol (herein referred to as a sterol ester), which may be created by the formal condensation of the carboxy group of any carboxylic acid with the 3-hydroxy group of a sterol. An ester is a chemical compound derived from an acid in which at least one hydroxyl group is replaced by an alkoxyl group. Sterol esters may be naturally occurring, e.g., DHEA sulfate or synthetic sterol ester e.g., estradiol valerate.

In some embodiments, the sterol ester may be a sulfate-based sterol ester. Certain sulfur-based sterol esters have a sulfamate or sulfonamide moiety as the ester, typically at the C3 and/or C17β positions. Examples include the estradiol esters estradiol sulfamate (E2MATE; also a potent steroid sulfatase inhibitor) and EC508 (estradiol 17β-(1-(4-(aminosulfonyl)benzoyl)-L-proline)), the testosterone ester EC586 (testosterone 17β-(1-((5-(aminosulfonyl)-2-pyridinyl)carbonyl)-L-proline)), sulfonamide esters of levonorgestrel and/or etonogestrel.

### Cationic Lipid

In some embodiments, a lipid of the disclosure may be a cationic lipid. As used herein, the term cationic lipid refers to a lipid that may be protonated at physiological pH.

In some embodiments, the cationic lipid is an ionizable cationic lipid, unsaturated cationic lipid, saturated cationic lipid, multivalent cationic lipid, amino lipid, or any combination thereof.

Non-limiting examples of a suitable cationic lipid include O′I,O′1-(2-(7-dodecyl-14-methyl-3,9-dioxo-2,4,8,10-tetraoxa-14-azapentadecyl)propane-1,3-diyl)8-dimethyldioctanedioate, N,N-dimethyl-1-[(1S,2S)-2-{[(1R,2R)-2-pentylcyclopropyl]methyl}cyclopropyl]nonadecan-10-amine, N,N-dimethyl-1-[(1S,2R)-2-octylcyclopropyl]nonadecan-10-amine, N,N-dimethyl-1-[(1S,2R)-2-octylcyclopropyl]hexadecan-8-amine, N,N-dimethyl-1-[(1R,2S)-2-undecylcyclopropyl]tetradecan-5-amine, Unknown (75-016B), trehalose6′6′-dibehenate (TDB), trans-1-methyl-3-[(9Z)-octadec-9-en-1-yloxy]-4-(octyloxy)pyrrolidine, trans-3-[({3}7-dimethyloctyl)oxy]-1-methyl-4˜[(9Z,12Z)-octadeca-9512-dien-1-yloxyjpyrrolidine, trans-1-methylpyrrolidine-3,4-diyl)bis(methylene)(9Z,9′Z,12Z,12′Z)-bis(octadeca-9,12-dienoate) (CL-4), trans-1-methyl-3-[(12Z)-octadec-12-en-1-yloxy]-4-(octyloxy)pyrrolidine, trans-1-Methyl-3-[((9Z,12Z)-octadeca-9,12-dienyl)oxy]-4-octyloxy-pyrrolidine, trans-1-methyl-3,4-bis(((Z)-octadeca-9-enoyloxy)methyl)pyrrolidine, trans-1-methyl-3,4-bis(((Z)-hexadec-9-enoyloxy)methyl)pyrrolidine, trans-1-methyl-3,4-bis(((9Z,12Z)-octadeca-9,12-dienoyloxy)methyl)pyrrolidine, trans-1-Methyl-3,4-bis((((Z)-octadec-9-en-1-yl)oxy)methyl)pyrrolidine (CL-3), TetradecylN-(2-(dinonylamino)ethyl)-N-nonylglycinate, stearylamine (STA), S-Pentyl4-((2-(4-(N-(2-(dinonylamino)ethyl)-N-nonylglycyl)piperazin-1-yl)-2-oxoethyl)(nonyl)amino)butanethioate, Propyl6-((2-(I-(N-(2-(dinonylamino)ethyl)-N-nonylglycyl)piperidin-4-yl)ethyl)(nonyl)amino)hexanoate, Propyl6-((2-(4-(N-(2-(dinonylamino)ethyl)-N-nonylglycyl)piperazin-1-yl)-2-oxoethyl)(nonyl)amino)hexanoate, Propyl6-((2-(4-(N-(2-(dinonylamino)ethyl)-N-nonylglycyl)piperazin-2-oxoethyl)(nonyl)amino)hexanoate, Propyl6-((2-(1-(N-(2-(dinonylamino)ethyl)-N-nonylglycyl)piperidin-4-yl)ethyl)(nonyl)amino)hexanoate, poly{4-((2-(dimethylamino)ethyl)thio)tetrahydro-2H-pyran-2-one}-r-poly{4-(octylthio)tetrahydro-2H-pyran-2-one}(A7), phosphatidylcholines (PC), Pentyl8,1l-dimethyl-5,14,17-trinonyl-7,12-dioxo-5,8,11,14,17-pentaazahexacosanoate2-((2-(Dinonylamino)ethyl)(nonyl)aniino)-N-methyl-N-(2-(methylandno)ethyl)acetami, Pentyl8-((2-(I-(N-(2-(dinonylamino)ethyl)-N-nonylglycyl)pyrrolidin-3-yl)ethyl)(tetradecyl)amino)octanoate/er/-Butyl3-(2-(tetradecylamino)ethyl)pyrrolidine-I-carboxylate, Pentyl8-((2-(I-(N-(2-(dinonylamino)ethyl)-N-nonylglycyl)piperidin-4-yl)ethyl)(tetradecyl)amino)octanoate, Pentyl8-((2-(I-(N-(2-(dinonylamino)ethyl)-N-nonylglycyl)piperidin-3-yl)ethyl)(tetradecyl)amino)octanoate, Pentyl8-((2-(4-(N-(2-(dinonylamino)ethyl)-N-nonylglycyl)piperazin-2-oxoethyl)(tetradecyl)amino)octanoate, Pentyl6-(dodecyl(2-(dodecyl(2-hydroxyethyl)amino)ethyl)amino)hexanoate (20-2), Pentyl6-((2-(dinonylamino)ethyl)(2-hydroxyethyl)amino)hexanoate (20-6), Pentyl6-((2-(didodecylamino)ethyl)(2-hydroxyethyl)amino)hexanoate (20-3), Pentyl6-((2-(4-(2-((2-(ditetradecylamino)ethyl)(tetradecyl)amino)ethyl)piperazin-1-yl)ethyl)(dodecyl)amino)hexanoate, Pentyl6-((2-(4-(2-((2-(didodecylamino)ethyl)(dodecyl)amino)ethyl)piperazin--yl)ethyl)(dodecyl)amino)hexanoateStep1:Pentyl6-bromohexanoate, Pentyl6-((2-(4-(2-((2-(didodecylamino)ethyl)(dodecyl)amino)ethyl)piperazin-1-yl)ethyl)(dodecyl)amino)hexanoate, Pentyl4-(nonyl(2-(4-(N-nonyl-N-(2-(nonyl(4-oxo-4-(pentyloxy)butyl)amino)ethyl)glycyl)piperazin--yl)-2-oxoethyl)amino)butanoate, Pentyl4-(nonyl(2-(4-(N-nonyl-N-(2-(nonyl(4-oxo-4-(pen1yloxy)buryl)amino)ethyl)glycyl)piperazin-1-yl)-2-oxoethyl)amino)butanoate, Pentyl4-((3-(I-(3-((2-(dinonylamino)ethyl)(nonyl)amino)propanoyl)piperidin-4-yl)propyl)(nonyl)amino)buta\hat{ }, Pentyl4-((3-(4-(3-((2-(dinonylamino)ethyl)(nonyl)amino)propanoyl)piperazin--yl)-3-oxopropyl)(nonyl)amino)butanoate, Pentyl4-((3-(4-(3-((2-(dinonylamino)ethyl)(nonyl)amino)propanoyl)piperazin-1-yl)-3-oxopropyl)(nonyl)amino)butanoate, Pentyl4-((3-(1-(3-((2-(dinonylamino)ethyl)(nonyl)amino)propanoyl)piperidin-4-yl)propyl)(nonyl)amino)butano, Pentyl4-((2-(I-(N-(2-(dinonylamino)ethyl)-N-nonylglycyl)pyrrolidin-3-yl)ethyl)(nonyl)amino)butanoate, Pentyl4-((2-(I-(N-(2-(dinonylamino)ethyl)-N-nonylglycyl)piperidin-4-yl)ethyl)(nonyl)amino)butanoate, Pentyl4-((2-(I-(N-(2-(dinonylamino)ethyl)-N-nonylglycyl)piperidin-3-yl)ethyl)(nonyl)amino)butanoate, Pentyl4-((2-(4-(N-(2-(dinonylamino)ethyl)-N-nonylglycyl)piperazin-I-yl)-2-oxoethyl)(nonyl)amino)butanoate, Pentyl4-((2-(4-(N-(2-(dinonylamino)ethyl)-N-nonylglycyl)piperazin-2-oxoethyl)(nonyl)amino)butanoate, Pentyl4-((2-(1-(N-(2-(dinonylamino)ethyl)-N-nonylglycyl)pyrrolidin-3-yl)ethyl)(nonyl)amino)butanoate, Pentyl4-((2-(1-(N-(2-(dinonylamino)ethyl)-N-nonylglycyl)piperidin-4-yl)ethyl)(nonyl)amino)butanoate, Pentyl4-((2-(1-(N-(2-(dinonylamino)ethyl)-N-nonylglycyl)piperidin-3-yl)ethyl)(nonyl)amino)butanoate, Pentyl4-(((1-(N-(2-(dinonylamino)ethyl)-N-nonylglycyl)pyrrolidin-3-yl)methyl)(nonyl)amino)butanoate, Pentyl4-(((1-(N-(2-(dinonylamino)ethyl)-N-nonylglycyl)pyrrolidin-3-yl)methyl)(nonyl)amino)butanoate, palmitoyi-oieoyl-nor-arginine (PONA), NonylN-(2-(dinonylamino)ethyl)-N-nonylglycinate, Nonyl8-((2-(didodecylamino)ethyl)(2-hydroxyethyl)amino)octanoate (20-8), N-Nonyl-N-(2-(piperazin-1-yl)ethyl)nonan-1-amine (19-1), NNN2-Trinonyl-N2-(2-(piperazin-1-yl)ethyl)ethane-1,2-diamine, NN1,N2-Tridodecyl-N2-(2-(piperazin-1-yl)ethyl)ethane-1,2-diamine, N-methyl-N,N-bis(2-((Z)-hexadec-9-enyloxy)ethyl)amine (CL-2), N-methyl-2-(((9Z,12Z)-octadeca-9,12-dien-1-yl)oxy)-N-(2-((((9Z,12Z)-octadeca-9,12-diene-1-yl)oxy)ethyl)ethan-1-amine (CL-11), NI,N19-bis((S,23E,25E,27E,29E)-16-((2E,4E,6E,8E)-3,7-dimethyl-9-(2,6,6-trimethylcyclo-hex-I-en-1-yl)nona-2,4,6,8-tetraenamido)-24,28-dimethyl-15,22-dioxo-30-(2,6,6-trimethylcyclohex-1-en-1-yl)-4,7,10-trioxa-14,21-diazatriaconta-23,25,27,29-tetraen-1-yl)-4,7,10,13,16-pentaoxanonadecane-1,19-diamide (diVA-PEG-diVA), N-Dodecyl-N-(2-(piperazin-1-yl)ethyl)dodecan-1-amine (19-2), N-Dodecyl-N-(2-(4-methylpiperazin-1-yl)ethyl)dodecan-1-amine1ntermediate1:2-(Didodecylamino)ethan-1-ol (19-4), N-Dodecyl-N-(2-(4-(4-methoxybenzyl)piperazin-1-yl)ethyl)dodecan-1-amine (19-5), N-decyl-2-((2-(dinonylamino)ethyl)(nonyl)amino), N2-[N2,N5-bis(3-aminopropyl)-L-ormithyl]-N,N-dioctadecyl-L-glutaminetetrahydrotrifluoroacetate (GC33), N1-[2-(didodecylamino)ethyl]-N1,N4,N4-tridodecyl-1,4-piperazinediethanamine (KL22), N1,N1,N2-Trinonyl-N2-(2-(piperazin-1-yl)ethyl)ethane-1,2-diamine, N1,N1,N2-Trihexyl-N2-(2-(piperazin-I-yl)ethyl)ethane-1,2-diamine, N1,N1,N2-Trihexyl-N2-(2-(piperazin-1-yl)ethyl)ethane-1,2-diamine, N1,N1,N2-Tridodecyl-N2-(2-(piperazin-1-yl)ethyl)ethane-1,2-diamine, N1,N1,N2-Tridodecyl-N2-(2-(4-(2-(dodecyl((9Z,12Z)-octadeca-9,12-dien-1-yl)amino)ethyl)piperazin-1-yl)ethyl)ethane-1,2-diamine, N1,N1,N2-Tridodecyl-N2-(2-(4-(2-(dodecyl((9Z,12Z)-octadeca-9,12-dien-1-yl)amino)ethyl)piperazin-1-yl)ethyl)ethane-1,2-diamine, N1,N1,N2-Tri((Z)-octadec-9-en-1-yl)-N2-(2-(piperazin-I-yl)ethyl)ethane-1,2-diamine, N1,N1,N2-Tri((Z)-octadec-9-en-1-yl)-N2-(2-(piperazin-1-yl)ethyl)ethane-1,2-diamine, N1,N1,N2-Tri((9Z,12Z)-octadeca-9,12-dien-1-yl)-N2-(2-(piperazin-1-yl)ethyl)ethane-1,2-diamine, N1,N1,N2-Tri((9Z,12Z)-octadeca-9,12-dien-1-yl)-N2-(2-(piperazin-1-yl)ethyl)ethane-1,2-diamine, N1-(3-(3-(dinonylamino)propoxy)propyl)-N1,N2,N2-trinonylethane-1,2-diamine, N1-(2-(Piperazin-I-yl)ethyl)-N1,N2,N2-tritetradecylethane-1,2-diamine, N1-(2-(dinonylamino)ethyl)-NN8,N8-trinonyloctane-1,8-diamine, N1-(2-(4-(2-(Ditetradecylamino)ethyl)piperazin-1-yl)ethyl)-N1,N2,N2-tritetradecylethane-1,2-diamine, N1-(2-(4-(2-(Ditetradecylamino)ethyl)piperazin-1-yl)ethyl)-N1,N2,N2-trinonylethane-1,2-diamine, N1-(2-(4-(2-(Ditetradecylamino)ethyl)piperazin--yl)ethyl)-N1,N2,N2-tridodecylethane-1,2-diamine, N1-(2-(4-(2-(Ditetradecylamino)ethyl)piperazin-1-yl)ethyl)-N1,N2,N2-tritetradecylethane-1,2-diamine, N1-(2-(4-(2-(Ditetradecylamino)ethyl)piperazin-1-yl)ethyl)-N1,N2,N2-tridodecylethane-1,2-diamine, N1-(2-(4-(2-(Dioctylamino)ethyl)piperazin--yl)ethyl)-N1,N2\hat{ }V2-tridodecylethane-1,2-diamine, N1-(2-(4-(2-(Dioctylamino)ethyl)piperazin--yl)ethyl)-N1,N2,N2-tridodecylethane-1,2-diamine, N1-(2-(4-(2-(Dinonylamino)ethyl)piperazin-1-yl)ethyl)-N1,N2\hat{ }V2-tritetradecylethane-1,2-diamine, N1-(2-(4-(2-(Dinonylamino)ethyl)piperazin-1-yl)ethyl)-N1,N2,N2-trinonylethane-1,2-diamine, N1-(2-(4-(2-(Dinonylamino)ethyl)piperazin--yl)ethyl)-N1,N2,N2-tridodecylethane-1,2-diamine, N1-(2-(4-(2-(Dinonylamino)ethyl)piperazin-1-yl)ethyl)-N1,N2,N2-tritetradecylethane-1,2-diamine, N1-(2-(4-(2-(Dihexylamino)ethyl)piperazin-1-yl)ethyl)-N1,N2,N2-tridodecylethan-1,2-diamine, N1-(2-(4-(2-(Didodecylamino)ethyl)piperazin-I-yl)ethyl)-trihexylethane-1,2-diamine, N1-(2-(4-(2-(Didodecylamino)ethyl)piperazin--yl)ethyl)-N1,N2,N2-tritetradecylethane-1,2-diamine, N1-(2-(4-(2-(Didodecylamino)ethyl)piperazin--yl)ethyl)-N1,N2,N2-trinonylethane-1,2-diamine, N1-(2-(4-(2-(Didodecylamino)ethyl)piperazin--yl)ethyl)-N1,N2,N2-trihexylethane-1,2-diamine, N1-(2-(4-(2-(Didodecylamino)ethyl)piperazin--yl)ethyl)-N1,N2,N2-tri((Z)-octadec-9-en-1-yl)ethane-1,2-diamine, N1-(2-(4-(2-(Didodecylamino)ethyl)piperazin--yl)ethyl)-N1,N2,N2-tri((9Z,12Z)-octadeca-9,12-dien-1-yl)ethane-1,2-diamine, N1-(2-(4-(2-(Didodecylamino)ethyl)piperazin-1-yl)ethyl)-N1,N2,N2-tritetradecylethane-1,2-diamine, N1-(2-(4-(2-(Didodecylamino)ethyl)piperazin-1-yl)ethyl)-N1,N2,N2-tri((Z)-octadec-9-en-1-yl)ethane-1,2-diamine, N1-(2-(4-(2-(Didodecylamino)ethyl)piperazin-1-yl)ethyl)-N1,N2,N2-tri((9Z,12Z)-octadeca-9,12-dien-1-yl)ethane-1,2-diamine, N1-(2-(4-(2-(Di((Z)-octadec-9-en-1-yl)amino)ethyl)piperazin-1-yl)ethyl)-tridodecylethane-1,2-diamine, N1-(2-(4-(2-(Di((Z)-octadec-9-en-1-yl)amino)ethyl)piperazin-1-yl)ethyl)-N1,N2,N2-tridodecylethane-1,2-diamine, N1-(2-(4-(2-(Di((Z)-dodec-6-en-1-yl)amino)ethyl)piperazin--yl)ethyl)-N1,N2,N2-tridodecylethane-1,2,-diamine, N1-(2-(4-(2-(Di((Z)-dodec-6-en-1-yl)amino)ethyl)piperazin-1-yl)ethyl)-N1,N2,N tridodecylethane-1,2-diamine, N1-(2-(4-(2-(Di((9Z,12Z)-octadeca-9,12-dien-1-yl)amino)ethyl)piperazin-1-yl)ethyl)-N1,N2,N2-tridodecylethane-1,2-diamine, N‘-methyl-N’,N,″N″-tris((2E.6E)-3.7.11-trimethyldodeca-2.6.10-trien-1-vnpropane-1,3-diamine, N-[I-(2,3-dimyristyloxy)propyl]-N,N-dimethyl-N-(2-hydroxyethyl)ammoniumbromide (DMRIE), N-[5′-(2′,3′-dioleoyl)uridine]-N′,N′,N′-trimethylammoniumtosylate (DOTAU), N,N-distearyl-N,N-dimethylammoniumbromide (DDAB), N,N-dioleyl-N,N-dimethylammoniumchloride (DODAC), N,N-di-n-octadecyl,N-methyl-N-(2-guanidinyl)ethylammoniumchloride (Lipid 2), N,N-di-n-Ictradecyl,N-methyl-N-(2-guanidinyl)ethylammonium (Lipid 1), N,N-dimethylheptacosan-10-amine, N,N-dimethyl-3-{7-[(1S,2R)-2-octylcyclopropyl]heptyl}dodecan-1-amine, N,N-dimethyl-3-{[(9Z,12Z)-octadeca-9,12-dien-1-yloxy]methyl}dodecan-1-amine, N,N-Dimethyl-3,4-dioleyloxybenzylamine (DMOBA), N,N-dimethyl-21-[(1S,2R)-2-octylcyclopropyl]henicosan-10-amine, N,N-dimethyl-2-{[(9Z,12Z)-octadeca-9,12-dien-1-yloxy]methyl}undecan-1-amine, N,N-dimethyl-2,3-dioleyloxypropylamine (DODMA), N,N-dimethyl-2,3-bis(((9Z,12Z)-octadeca-9,12-dien-1-yl)oxy)propan-1-amine (CL-1), N,N-dimethyl-1-[(1S,2R)-2-octylcyclopropyl]pentadecan-8-amine, N,N-dimethyl-1-[(1S,2R)-2-octylcyclopropyl]heptadecan-8-amine, N,N-dihydroxyethylN,N-dioctadecylammoniumchloride (DODEAC), N,N-dihydroxyethylmethyl-N-2-(cholesteryloxycarbonylamino)ethylammoniumbromide (BHEM-Chol), N,N-bis-(2-hydroxyethyl)-N-methyl-N-(2-cholesteryloxycarbonylamino-ethyl)ammoniumbromide (BHEM-Choli), N,N,N-trimethyl-5-oxo-5-(3-((3-pentyloctanoyl)oxy)-2,2-bis(((3-pentyloctanoyl)oxy)methyl)propoxy)pentane-1-Aminiumiodide3-((5-(dimethylamino)pentanoyl)oxy)-2,2-bis(((3-pentyloctanoyl)oxy)methyl)propyl3-pentyloctanoate, N,2-Dimethyl-1,3-bis(((9Z,12Z)-octadeca-9,12-dien-1-yl)oxy)propan-2-amine (CL-9), N-(α-trimethylammonioacetyl)-didodecyl-D-glutamatechloride (TMAG), N-(2-(dimethylamino)ethyl)-4,5-bis(dodecylthio)pentanamide (DEDPA), N-(2-(Didodecylamino)ethyl)-N-dodecylglycine (20-1), N-(1-(2,3-dioleyloxy)propyl)-N,N,N-trimethylammoniumchloride (DOTMA), N-(1-(2,3-dioleoyloxy)propyl)-N,N,N-trimethylammoniumchloride (DOTAP), monopalmitoylphosphatidylcholin (MPPC), mon-omycolylglycerol (MMG), Methylpyridiyl-dialkyl-carboxylicacid (MPDACA), Methyldi((9Z,12Z)-octadeca-9,12-dienyl)amine, methyl9-{[4-(dimethylamino)butanoyl]oxy}-16-(2-octylcyclopropyl)hexadecanoate, methyl8-{2-[II-(dimethylamino)octadecyl]cyclopropyl}octanoate, methyl8-{2-[II-(dimethylamino)nonadecyl]cyclopropyl}octanoate, methyl8-{2-[II-(dimethylamino)icosyl]cyclopropyl}octanoate, methyl8-{2-[II-(dimethylamino)heptadecyl]cyclopropyl}octanoate(Compound18), methyl8-{2-[9-(dimethylamino)pentadecyl]cyclopropyl}octanoate, methyl8-{2-[9-(dimethylamino)octadecyl]cyclopropyl}octanoate, methyl8-{2-[9-(dimethylamino)hexadecyl]cyclopropyl}octanoate, methyl8-{2-[9-(dimethylamino)heptadecyl]cyclopropyl}octanoate, methyl8-[2-{]11-\hat{ }2-(dimethylamino)ethyl]icosyl}cyclopropyl)octanoate, methyl8-[2-(II-{[4-(dimethylamino)butanoyl]oxy}octadecyl)cyclopropyl]octanoate, methyl8-[2-(II-{[4-(dimethylamino)butanoyl]oxy}nonadecyl)cyclopropyl]octanoate, methyl8-[2-(II-{[4-(dimethylamino)butanoyl]oxy}icosyl)cyclopropyl]octanoate, methyl8-[2-(9-{[4-(dimethylamino)butanoyl]oxy}pentadecyl)cyclopropyl]octanoate, methyl8-[2-(9-{[4-(dimethylamino)butanoyl]oxy}octadecyl)cyclopropyl]octanoate, methyl8-[2-(9-{[4-(dimethylamino)butanoyl]oxy}hexadecyl)cyclopropyl]octanoate, methyl8-[2-(9-{[4-(dimethylamino)butanoyl]oxy}heptadecyl)cyclopropyl]octanoate, methyl8-(2-{ILI-;2-(dimethylamino)ethyl]octadecyl}cyclopropyl)octanoate, methyl8-(2-{II-[(dimethylamino)methyl]octadecyl}cyclopropyl)octanoate, methyl8-(2-{II-[(dimethylamino)methyl]nonadecyl}cyclopropyl)octanoate, methyl8-(2-{II-[(dimethylamino)methyl]icosyl}cyclopropyl)octanoate, methyl8-(2-{II-[(dimethylamino)methyl]heptadecyl}cyclopropyl)octanoate, methyl8-(2-{I11-″2-(dimethylamino)ethyl]nonadecyl}cyclopropyl)octanoate, methyl8-(2-{9-[2-(dimethylamino)ethyl]pentadecyl}cyclopropyl)octanoate, methyl8-(2-{9-[2-(dimethylamino)ethyl]octadecyl}cyclopropyl)octanoate, methyl8-(2-{9-[2-(dimethylamino)ethyl]hexadecyl}cyclopropyl)octanoate, methyl8-(2-{9-[2-(dimethylamino)ethyl]heptadecyl}cyclopropyl)octanoate, methyl8-(2-{9-[(dimethylamino)methyl]pentadecyl}cyclopropyl)octanoate, methyl8-(2-{9-[(dimethylamino)methyl]octadecyl}cyclopropyl)octanoate, methyl8-(2-{9-[(dimethylamino)methyl]hexadecyl}cyclopropyl)octanoate, methyl8-(2-{9-[(dimethylamino)methyl]heptadecyl}cyclopropyl)octanoate, methyl8-(2-{I11-,2-(dimethylamino)ethyl]heptadecyl}cyclopropyl)octanoate, methyl8-(2-(dimethylamino)-3-((9Z,12Z)-octadeca-9,12-dien-I-yloxy)propoxy)octanoate, methyl8-(2-(dimethylamino)-3-((8-(2-octylcyclopropyl)octyl)oxy)propoxy)octanoate, methyl8-(2-(dimethylamino)-3-((8-(2-(6-methoxy-6-oxohexyl)cyclopropyl)octyl)oxy)propoxy)octanoate, methyl8-(2-(dimethylamino)-3-((8-(2-((2-pentylcyclopropyl)methyl)cyclopropyl)octyl)oxy)propoxy)octanoate, methyl8-(2-(dimethylamino)-3-((6-((2-octylcyclopropyl)methoxy)-6-oxohexyl)oxy)propoxy)octanoate, Methyl8-((2-(I-(N-(2-(dinonylamino)ethyl)-N-nonylglycyl)piperidin-4-yl)ethyl)(nonyl)amino)octanoate, Methyl8-((2-(dinonylamino)ethyl)(2-(4-(dinonylglycyl)piperazin-1-yl)-2-oxoethyl)amino)octanoate, Methyl8-((2-(dinonylamino)ethyl)(2-(4-(dinonylglycyl)piperazin-1-yl)-2-oxoethyl)amino)octanoate, Methyl8-((2-(4-(N-(2-(dinonylamino)ethyl)-N-nonylglycyl)piperazin-I-yl)-2-oxoethyl)(nonyl)amino)octanoate, Methyl8-((2-(4-(N-(2-(dinonylamino)ethyl)-N-nonylglycyl)piperazin-1-yl)-2-oxoethyl)(nonyl)amino)octanoate, Methyl8-((2-(4-(N-(2-(Di((Z)-non-3-en-1-yl)amino)ethyl)-N-((Z)-non-3-en-1-yl)glycyl)piperazin-I-yl)-2-oxoethyl)(nonyl)amino)octanoatefert-Butyl4-(nonylglycyl)piperazine-1-carboxylate, Methyl8-((2-(4-(dinonylglycyl)piperazin-1-yl)-2-oxoethyl)(2-((8-methoxy-8-oxooctyl)(nonyl)amino)ethyl)amino)octanoate, Methyl8-((2-(4-(dinonylglycyl)piperazin-1-yl)-2-oxoethyl)(2-((8-methoxy-8-oxooctyl)(nonyl)amino)ethyl)amino)octanoate, Methyl8-((2-(1-(N-(2-(dinonylamino)ethyl)-N-nonylglycyl)piperidin-4-yl)ethyl)(nonyl)amino)octanoate, Methyl8-((2-((2-(4-(dinonylglycyl)piperazin-1-yl)-2-oxoethyl)(nonyl)amino)ethyl)(nonyl)amino)octanoate, Methyl8-((2-((2-(4-(dinonylglycyl)piperazin-1-yl)-2-oxoethyl)(nonyl)amino)ethyl)(nonyl)amino)octanoate, methyl7-(2-(dimethylamino)-3-((9Z,12Z)-octadeca-9,12-dien-1-yloxy)propoxy)heptanoate, methyl7-(2-(dimethylamino)-3-((8-(2-octylcyclopropyl)octyl)oxy)propoxy)heptanoate, methyl7-(2-(dimethylamino)-3-((8-(2-(6-methoxy-6-oxohexyl)cyclopropyl)octyl)oxy)propoxy)heptanoate, methyl7-(2-(dimethylamino)-3-((8-(2-((2-pentylcyclopropyl)methyl)cyclopropyl)octyl)oxy)propoxy)heptanoate, methyl7-(2-(dimethylamino)-3-((6-((2-octylcyclopropyl)methoxy)-6-oxohexyl)oxy)propoxy)heptanoate, methyl7-(2-(8-(2-(dimethylamino)-3-(octyloxy)propoxy)octyl)cyclopropyl)heptanoate, methyl6-{2-[II-(dimethylamino)icosyl]cyclopropyl}hexanoate, methyl6-[2-(II-{[4-(dimethylamino)butanoyl]oxy}icosyl)cyclopropyl]hexanoate, methyl6-(2-{II-[(dimethylamino)methyl]icosyl}cyclopropyl)hexanoate, methyl6-(2-{]11-\hat{ }2-(dimethylamino)ethyl]icosyl}cyclopropyl)hexanoate, methyl6-(2-(dimethylamino)-3-((9Z,12Z)-octadeca-9,12-dien-1-yloxy)propoxy)hexanoate, methyl6-(2-(dimethylamino)-3-((8-(2-octylcyclopropyl)octyl)oxy)propoxy)hexanoate, methyl6-(2-(dimethylamino)-3-((8-(2-((2-pentylcyclopropyl)methyl)cyclopropyl)octyl)oxy)propoxy)hexanoate, methyl6-(2-(dimethylamino)-3-((6-((2-octylcyclopropyl)methoxy)-6-oxohexyl)oxy)propoxy)hexanoate, methyl6-(2-(8-(3-(decyloxy)-2-(dimethylamino)propoxy)octyl)cyclopropyl)hexanoate, methyl6-(2-(8-(2-(dimethylamino)-3-(nonyloxy)propoxy)octyl)cyclopropyl)hexanoate, methyl6-(2-(8-(2-(dimethylamino)-3-(hexyloxy)propoxy)octyl)cyclopropyl)hexanoate, methyl6-(2-(8-(2-(dimethylamino)-3-(heptyloxy)propoxy)octyl)cyclopropyl)hexanoate, methyl6-(2-(8-(2-(dimethylamino)-3-(4-methoxy-4-oxobutoxy)propoxy)octyl)cyclopropyl)hexanoate, methyl6-(2-(8-(2-(dimethylamino)-3-((6-methoxy-6-oxohexyl)oxy)propoxy)octyl)cyclopropyl)hexanoate, methyl6-(2-(8-(2-(dimethylamino)-3-((5-methoxy-5-oxopentyl)oxy)propoxy)octyl)cyclopropyl)hexanoate, methyl5-(2-(dimethylamino)-3-((9Z,12Z)-octadeca-9,12-dien-1-yloxy)propoxy)pentanoate, methyl5-(2-(dimethylamino)-3-((8-(2-octylcyclopropyl)octyl)oxy)propoxy)pentanoate, methyl5-(2-(dimethylamino)-3-((8-(2-((2-pentylcyclopropyl)methyl)cyclopropyl)octyl)oxy)propoxy)pentanoate, methyl4-(2-(dimethylamino)-3-((9Z,12Z)-octadeca-9,12-dien-1-yloxy)propoxy)butanoate, methyl4-(2-(dimethylamino)-3-((8-(2-octylcyclopropyl)octyl)oxy)propoxy)butanoate, methyl4-(2-(dimethylamino)-3-((8-(2-((2-pentylcyclopropyl)methyl)cyclopropyl)octyl)oxy)propoxy)butanoate, Methyl12-((2-(I-(N-(2-(dinonylamino)ethyl)-N-nonylglycyl)pyrrolidin-3-yl)ethyl)(tetradecyl)amino)dodecanoatefert-Butyl3-(2-((12-methoxy-12-oxododecyl)(tetradecyl)amino)ethyl)pyrrolidine-I-carboxylate, Methyl12-((2-(I-(N-(2-(dinonylamino)ethyl)-N-nonylglycyl)piperidin-4-yl)ethyl)(tetradecyl)amino)dodecanoate, Methyl12-((2-(I-(N-(2-(dinonylamino)ethyl)-N-nonylglycyl)piperidin-3-yl)ethyl)(tetradecyl)amino)dodecanoate-Butyl3-(2-((12-methoxy-12-oxododecyl)(tetradecyl)amino)ethyl)piperidine-I-carboxylate, Methyl12-((2-(4-(N-(2-(dinonylamino)ethyl)-N-nonylglycyl)piperazin-I-yl)-2-oxoethyl)(tetradecyl)amino)dodecanoate, Methyl12-((2-(4-(2-((2-(didodecylamino)ethyl)(dodecyl)amino)ethyl)piperazin-1-yl)ethyl)(dodecyl)amino)dodecanoate, methyl10-{2-[7-(dimethylamino)hexadecyl]cyclopropyl}decanoate, methyl10-[2-(7-{[4-(dimethylamino)butanoyl]oxy}hexadecyl)cyclopropyl]decanoate, methyl10-(2-V-\hat{ }2-(dimethylamino)ethyl]hexadecyl}cyclopropyl)decanoate, methyl10-(2-{7-[(dimethylamino)methyl]hexadecyl}cyclopropyl)decanoate, methyl(HZ)-19-[(dimethylamino)methyl]octacos-II-enoate, methyl(9Z)-21-{[4-(dimethylamino)butanoyl]oxy}triacont-9-enoate, methyl(9Z)-21-{[4-(dimethylamino)butanoyl]oxy}octacos-9-enoate, methyl(9Z)-21-{[4-(dimethylamino)butanoyl]oxy}nonacos-9-enoate, methyl(9Z)-21-{[4-(dimethylamino)butanoyl]oxy}heptacos-9-enoate, methyl(9Z)-21-[2-(dimethylamino)ethyl]triacont-9-enoate, methyl(9Z)-21-[2-(dimethylamino)ethyl]octacos-9-enoate, methyl(9Z)-21-[2-(dimethylamino)ethyl]nonacos-9-enoate, methyl(9Z)-21-[2-(dimethylamino)ethyl]heptacos-9-enoate, methyl(9Z)-21-[(dimethylamino)methyl]triacont-9-enoate, methyl(9Z)-21-[(dimethylamino)methyl]octacos-9-enoate, methyl(9Z)-21-[(dimethylamino)methyl]nonacos-9-enoate, methyl(9Z)-21-[(dimethylamino)methyl]heptacos-9-enoate, methyl(9Z)-21-(dimethylamino)triacont-9-enoate, methyl(9Z)-21-(dimethylamino)octacos-9-enoate, methyl(9Z)-21-(dimethylamino)nonacos-9-enoate, methyl(9Z)-21-(dimethylamino)heptacos-9-enoate, methyl(9Z)-19-{[4-(dimethylamino)butanoyl]oxy}pentacos-9-enoate, methyl(9Z)-19-{[4-(dimethylamino)butanoyl]oxy}octacos-9-enoate, methyl(9Z)-19-{[4-(dimethylamino)butanoyl]oxy}hexacos-9-enoate, methyl(9Z)-19-{[4-(dimethylamino)butanoyl]oxy}heptacos-9-enoate, methyl(9Z)-19-[2-(dimethylamino)ethyl]pentacos-9-enoate, methyl(9Z)-19-[2-(dimethylamino)ethyl]octacos-9-enoate, methyl(9Z)-19-[2-(dimethylamino)ethyl]hexacos-9-enoate, methyl(9Z)-19-[2-(dimethylamino)ethyl]heptacos-9-enoate, methyl(9Z)-19-[(dimethylamino)methyl]pentacos-9-enoate, methyl(9Z)-19-[(dimethylamino)methyl]octacos-9-enoate, methyl(9Z)-19-[(dimethylamino)methyl]hexacos-9-enoate, methyl(9Z)-19-[(dimethylamino)methyl]heptacos-9-enoate, methyl(9Z)-19-(dimethylamino)pentacos-9-enoate, methyl(9Z)-19-(dimethylamino)octacos-9-enoate, methyl(9Z)-19-(dimethylamino)hexacos-9-enoate, methyl(9Z)-19-(dimethylamino)heptacos-9-enoate, methyl(7Z)-19-{[4-(dimethylamino)butanoyl]oxy}octacos-7-enoate, methyl(7Z)-19-[2-(dimethylamino)ethyl]octacos-7-enoate, methyl(7Z)-19-[(dimethylamino)methyl]octacos-7-enoate, methyl(7Z)-19-(dimethylamino)octacos-7-enoate, methyl(11Z)-19-{[4-(dimethylamino)butanoyl]oxy}octacos-11-enoate, methyl(11Z)-19-[2-(dimethylamino)ethyl]octacos-11-enoate, methyl(11Z)-19-(dimethylamino)octacos-11-enoate, methyl(19Z,22Z)-9-{[4-(dimethylamino)butanoyl]oxy}octacosa-19,22-dienoate, L-lysine-alpha-(2,3-dilauryloxy)propylamide, L-arginine-alpha-(2,3-dilauryloxy)propylamide, 1\hat{ }-Diarachidonyloxy-\hat{ }TV-dimethy\hat{ }-propyl-S-amine (DAraDMA), I,I′-(Piperazine-1,4-diyl)bis(5-(didecylamino)pentan-1-one, 1,1-(Piperazine-1,4-diyl)bis(4-(didecylamino)butan-1-one)fert-Butyl4-(didecylaminobutanoate, 1,3-Bis-(1,2-bis-tetradecyloxy-propyl-3-dimemylethoxyammoniumbromide)-propane-2-ol (PCL-2), I,2-disteroyl-3-trimethylammoniumpropane (DSTAP), I,2-Diphytanyloxy-3-(iV,7V-dimethyl)-propylamine (DPanDMA), I,2-dimyristoyl-3-trimethylammoniumpropane (DMTAP), 1,2-Dilinoleyloxy-3-trimethylaminopropane (DLinTMA), I,2-Dilinoleyloxy-3-(N-methylpiperazino)propane (DLinMPZ), I,2-Dilinoleoyl-3-trimethylaminopropane (DLinTAP), I,2-Dilauroyl-sn-Glicero-3-Phosphoethanolamine (DLPE), 1,2-Dilauroyl-sn-Glicero-3-Glycerol (DLPG), I,2-Didocosahexaenyloxy-(7V,N-dimethyl)-propyl-3-amine (DDocDMA), 1,17-bis(2-octylcyclopropyl)heptadecan-9-yl4-(dimethylamino)butanoate (CL), 1,17-bis(2-((2-pentylcyclopropyl)methyl)cyclopropyl)heptadecan-9-yl4-(dimethylamino)butanoate, I,-(piperazine-1,4-diyl)bis(2-(dinonylamino)ethan-1-one), I-(3-cholesteryl)-oxycarbonyl-aminomethylimidazole (CHIM), 1-(2-(Dinonylamino)ethyl)4-(2-((2-(dinonylamino)ethyl)(nonyl)amino)ethyl)cyclohexane-1,4-dicarboxylate2-(Dinonylamino)ethan-1-ol, imidazolecholesterolester (ICE), HydroSoyPC (HSPC), Histaminyl-Cholesterolhemisuccinat (HisChol), Heptyl6-(dodecyl(2-(dodecyl(2-hydroxyethyl)amino)ethyl)amino)hexanoate (20-7), Heptyl6-((2-(I-(N-(2-(dinonylamino)ethyl)-N-nonylglycyl)pyrrolidin-3-yl)ethyl)(tetradecyl)amino)hexanoatetot-Butyl3-(2-((6-(heptyloxy)-6-oxohexyl)(tetradecyl)amino)ethyl)pyrrolidine-1-carboxylate, Heptyl6-((2-(I-(N-(2-(dinonylamino)ethyl)-N-nonylglycyl)piperidin-4-yl)ethyl)(tetradecyl)amino)hexanoate, Heptyl6-((2-(I-(N-(2-(dinonylamino)ethyl)-N-nonylglycyl)piperidin-3-yl)ethyl)(tetradecyl)amino)hexanoate, Heptyl6-((2-(4-(N-(2-(dinonylamino)ethyl)-N-nonylglycyl)piperazin-I-yl)-2-oxoethyl)(tetradecyl)amino)hexanoate, Heptyl5-(4-(N-(2-(dinonylamino)ethyl)-N-nonylglycyl)piperazin-I-yl)-5-oxopentanoate5-(Heptloxy)-5-oxopentanoicacid, Heptyl5-(4-(N-(2-(dinonylamino)ethyl)-N-nonylglycyl)piperazin-I-yl)-5-oxopentanoate5-(Heptloxy)-5-oxopentanoic, heptatriaconta-6,9,28,31-tetraen-19-yl-4-(dimethylamino)-butanoate (CL01), Heptadecan-9-yl8-((2-hydroxyethyl)(tetradecyl)amino)octanoateRepresentative, Heptadecan-9-yl8-((2-hydroxyethyl)(8-(nonyloxy)-8-oxooctyl)amino)octanoate, Heptadecan-9-yl8-((2-(didodecylamino)ethyl)(2-hydroxyethyl)amino)octanoate (20-9), Guanidino-dialkyl-carboxylicacid (GUADACA), glycerylmono-oleate (GMO), ethyl8-{2-[II-(dimethylamino)octadecyl]cyclopropyl}octanoate, ethyl8-{2-[II-(dimethylamino)nonadecyl]cyclopropyl}octanoate, ethyl8-{2-[II-(dimethylamino)icosyl]cyclopropyl}octanoate, ethyl8-{2-[II-(dimethylamino)heptadecyl]cyclopropyl}octanoate, ethyl8-{2-[9-(dimethylamino)pentadecyl]cyclopropyl}octanoate, ethyl8-{2-[9-(dimethylamino)octadecyl]cyclopropyl}octanoate, ethyl8-{2-[9-(dimethylamino)hexadecyl]cyclopropyl}octanoate, ethyl8-{2-[9-(dimethylamino)heptadecyl]cyclopropyl}octanoate, ethyl8-{2-[7-(dimethylamino)hexadecyl]cyclopropyl}octanoate, ethyl8-{[4-(dimethylamino)butanoyl]oxy}-15-(2-octylcyclopropyl)pentadecanoate, ethyl8-(2-{l-[(dimethylamino)methyl]octadecyl}cyclopropyl)octanoate, ethyl8-(2-{l-[(dimethylamino)methyl]nonadecyl}cyclopropyl)octanoate, ethyl8-(2-{II-[(dimethylamino)methyl]icosyl}cyclopropyl)octanoate, ethyl8-(2-{II-[(dimethylamino)methyl]heptadecyl}cyclopropyl)octanoate, ethyl8-(2-{9-[(dimethylamino)methyl]pentadecyl}cyclopropyl)octanoate, ethyl8-(2-{9-[(dimethylamino)methyl]octadecyl}cyclopropyl)octanoate, ethyl8-(2-{9-[(dimethylamino)methyl]hexadecyl}cyclopropyl)octanoate, ethyl8-(2-{9-[(dimethylamino)methyl]heptadecyl}cyclopropyl)octanoate, ethyl8-(2-{7-[(dimethylamino)methyl]hexadecyl}cyclopropyl)octanoate, Ethyl7-((2-(I-(N-(2-(dinonylamino)ethyl)-N-nonylglycyl)piperidin-4-yl)ethyl)(nonyl)amino)heptanoate, Ethyl7-((2-(4-(N-(2-(dinonylamino)ethyl)-N-nonylglycyl)piperazin-I-yl)-2-oxoethyl)(nonyl)amino)heptanoate, Ethyl7-((2-(4-(N-(2-(dinonylamino)ethyl)-N-nonylglycyl)piperazin-1-yl)-2-oxoethyl)(nonyl)amino)heptanoate, Ethyl7-((2-(1-(N-(2-(dinonylamino)ethyl)-N-nonylglycyl)piperidin-4-yl)ethyl)(nonyl)amino)heptanoate, ethyl6-{2-[9-(dimethylamino)pentadecyl]cyclopropyl}hexanoate, ethyl6-{2-[9-(dimethylamino)octadecyl]cyclopropyl}hexanoate, ethyl6-{2-[9-(dimethylamino)hexadecyl]cyclopropyl}hexanoate, ethyl6-{2-[9-(dimethylamino)heptadecyl]cyclopropyl}hexanoate, ethyl6-[2-(9-{[4-(dimethylamino)butanoyl]oxy}octadecyl)cyclopropyl]hexanoate, ethyl6-(2-{9-[(dimethylamino)methyl]pentadecyl}cyclopropyl)hexanoate, ethyl6-(2-{9-[(dimethylamino)methyl]octadecyl}cyclopropyl)hexanoate, ethyl6-(2-{9-[(dimethylamino)methyl]hexadecyl}cyclopropyl)hexanoate, ethyl6-(2-{9-[(dimethylamino)methyl]heptadecyl}cyclopropyl)hexanoate, ethyl4-{2-[II-(dimethylamino)icosyl]cyclopropyl}butanoate, ethyl4-(2-{II-[(dimethylamino)methyl]icosyl}cyclopropyl)butanoate, ethyl(9Z)-21-[(dimethylamino)methyl]triacont-9-enoate, ethyl(9Z)-21-[(dimethylamino)methyl]octacos-9-enoate, ethyl(9Z)-21-[(dimethylamino)methyl]nonacos-9-enoate, ethyl(9Z)-21-[(dimethylamino)methyl]heptacos-9-enoate, ethyl(9Z)-21-(dimethylamino)triacont-9-enoate, ethyl(9Z)-21-(dimethylamino)octacos-9-enoate, ethyl(9Z)-21-(dimethylamino)nonacos-9-enoate, ethyl(9Z)-21-(dimethylamino)heptacos-9-enoate, ethyl(9Z)-19-[(dimethylamino)methyl]pentacos-9-enoate, ethyl(9Z)-19-[(dimethylamino)methyl]octacos-9-enoate, ethyl(9Z)-19-[(dimethylamino)methyl]hexacos-9-enoate, ethyl(9Z)-19-[(dimethylamino)methyl]heptacos-9-enoate, ethyl(9Z)-19-(dimethylamino)pentacos-9-enoate, ethyl(9Z)-19-(dimethylamino)octacos-9-enoate, ethyl(9Z)-19-(dimethylamino)hexacos-9-enoate, ethyl(9Z)-19-(dimethylamino)heptacos-9-enoate, ethyl(9Z)-17-[(dimethylamino)methyl]hexacos-9-enoate, ethyl(9Z)-17-(dimethylamino)hexacos-9-enoate, ethyl(7Z)-17-{[4-(dimethylamino)butanoyl]oxy}hexacos-7-enoate, ethyl(7Z)-17-[2-(dimethylamino)ethyl]hexacos-7-enoate, ethyl(7Z)-17-[(dimethylamino)methyl]tricos-7-enoate, ethyl(7Z)-17-[(dimethylamino)methyl]tetracos-7-enoate, ethyl(7Z)-17-[(dimethylamino)methyl]pentacos-7-enoate, ethyl(7Z)-17-[(dimethylamino)methyl]hexacos-7-enoate, ethyl(7Z)-17-(dimethylamino)tricos-7-enoate, ethyl(7Z)-17-(dimethylamino)tetracos-7-enoate, ethyl(7Z)-17-(dimethylamino)pentacos-7-enoate, ethyl(7Z)-17-(dimethylamino)hexacos-7-enoate, ethyl(5Z)-17-[(dimethylamino)methyl]hexacos-5-enoate, ethyl(5Z)-17-(dimethylamino)hexacos-5-enoate, ethyl(18Z,21Z)-8-{[4-(dimethylamino)butanoyl]oxy}heptacosa-18,21-dienoate, ditetradecyltrimethylammonium (DTDTMA), distearyldimethylammonium (DSDMA), disclosedinNon-PatentLiteraturel11 (YSK05), Dipentyl6,6′-((2-(dodecyl(2-hydroxyethyl)amino)ethyl)azanediyl)dihexanoate (20-4), Dipentyl6,6′-((2-(4-(2-((2-(didodecylamino)ethyl)(dodecyl)amino)ethyl)piperazin-I-yl)ethyl)azanediyl)dihexanoate, Dipentyl4,4′-((2-(4-(N-(2-(dinonylarnino)ethyl)-N-nonylglycyl)piperazin-I-yl)-2-oxoethyl)azanediyl)dibutyrate, Dipentyl4,4′-((2-(4-(N-(2-(dinonylamino)ethyl)-N-nonylglycyl)piperazin-1-yl)-2-oxoethyl)azanediyl)dibutyrate, dipalmitoyl-4-aminobutyricacid (DPFAB), dioleylphosphatidyluridinephosphatidylcholine (DOUPC), Dioleyl-N,N-DimethylGlycine (DL-048), dioleoyl-4-aminobutyricacid (DOFAB), Dioctadecylamidoglycylspermine (DOGS), Dioctadecyl-(2-hydroxy1-3-propylamino)aminopolylysine (Lipid T), Dinonyl8,8′-((2-(dodecyl(2-hydroxyethyl)amino)ethyl)azanediyl)dioctanoate (20-10), dimyristoyltrimethylammoniumpropane (Amino Lipid 6), dimyristoylphosphatidylserine (DMPS), dimethyldioctadecylammoniumbromide (DDA), dimethyldioctadecylammonium (DDA), Dimethyl12,12′-((2-(4-(2-((2-(didodecylamino)ethyl)(dodecyl)amino)ethyl)piperazin-I-yl)ethyl)azanediyl)didodecanoate, dimethyl(9Z)-19-{[4-(dimethylamino)butanoyl]oxy}heptacos-9-enedioate, Dilinoleyloxy3-piperidinopropylamine (DLinPip), dilinoleylmethyl4-(dimethylamino)propylether), dilinoleylmethyl4-(dimethylamino)butylether), dilinoleoyl-4-aminobutyricacid (DLinFAB), DiLin-N-Methylpiperazine (DL-033), DiLin-N,N-DimethylGlycine (DL-036), Diheptyl6,6′-((2-((6-(heptyloxy)-6-oxohexyl)(2hydroxyethyl)amino)ethyl)azanediyl)dihexanoate (20-5), didodecyl6,6′-((5-((dimethylamino)methyl)-1,3-phenylene)bis(oxy))dihexanoate, didecyl8,8′-((5-((dimethylamino)methyl)-1,3-phenylene)bis(oxy))dioctanoate, Diaceticacid(7R,9Z,26Z,29R)-18-({[3-(dimethylamino)propoxy]carbonyl}oxy)pentatriaconta-9,26-diene-7,29-diyl, Di(heptadecan-9-yl)8,8′-(II,24,29,42-tetraoxo-10,25,28,43-tetraoxa-19,34-diazadopentacontane-19,34-diyl)dioctanoate, Di(heptadecan-9-yl)8,8′-(26,28-dimethyl-II,24,30,43-tetraoxo-10,25,29,44-tetraoxa-19,35-diazatripentacontane-19,35-diyl)dioctanoate, Di(heptadecan-9-yl)8,8′-(26,27-dimethyl-II,24,29,42-tetraoxo-10,25,28,43-tetraoxa-19,34-diazadopentacontane-19,34-diyl)dioctanoate, Di(heptadecan-9-yl)8,8′-((piperazine-1,4-diylbis(5-oxopentane-5,I-diyl))bis((8-(nonyloxy)-8-oxooctyl)azanediyl))dioctanoate, Di(heptadecan-9-yl)15,19-dimethyl-9,25-bis(8-(nonyloxy)-8-oxooctyl)-14,20-dioxo-9,15,19,25-tetraazatritriacontanedioate, Di(heptadecan-9-yl)15,18-dimethyl-9,24-bis(8-(nonyloxy)-8-oxooctyl)-14,19-dioxo-9,15,18,24-tetraazadotriacontanedioate, Di(heptadecan-9-yl)15,18-diethyl-9,24-bis(8-(nonyloxy)-8-oxooctyl)-14,19-dioxo-9,15,18,24-tetraazadotriacontanedioate, di((9Z,12Z)-octadeca-9,12-dien-1-yl)3-(((2-(dimethylamino)ethoxy)carbonyl)amino)pentanedioate, di((9Z,12Z)-octadeca-9,12-dien-1-yl)5,5′-((5-((dimethylamino)methyl)-1,3-phenylene)bis(oxy))dipentanoate, describedinU.S.ProvisionalApplicationNo.61/384,050 (MC3 Thioester), cis-1-methyl-3-[(9Z,12Z)-octadeca-9,12-dien-1-yloxy]-4-(octyloxy)pyrrolidine, Cholesteryl-succinyl Silane (C2), cholesteryl3-(dimethylamino)propanoate (CL06), cholesteryl2-(dimethylamino)acetate (CL08), Cholest-5-en-3-ol(3P)—,3-[(3-aminopropyl)[4-[(3-aminopropyl)amino]butyl]carbamate](GL67), cetyltrimethylammoniumbromide (CTAB), C2:N,N-dihexadecyl-N′-(3-triethoxysilylpropyl)succinamide (CL3), Butyl5-((2-(I-(N-(2-(dinonylamino)ethyl)-N-nonylglycyl)piperidin-4-yl)ethyl)(nonyl)amino)pentanoate, Butyl5-((2-(4-(N-(2-(dinonylamino)ethyl)-N-nonylglycyl)piperazin-I-yl)-2-oxoethyl)(nonyl)amino)pentanoate, Butyl5-((2-(4-(N-(2-(dinonylamino)ethyl)-N-nonylglycyl)piperazin-1-yl)-2-oxoethyl)(nonyl)amino)pentanoate, Butyl5-((2-(1-(N-(2-(dinonylamino)ethyl)-N-nonylglycyl)piperidin-4-yl)ethyl)(nonyl)amino)pentanoate, Bis(2-(((9Z,12Z)-octadeca-9,12-dien-1-yl)oxy)ethyl)amine (CL-16), bis(1,3-bis(octanoyloxy)propan-2-yl)0,0′-((5-((dimethylamino)methyl)-1,3-phenylene)bis(methylene))disuccinate, 2,2-dilinoleyl-4-(2-dimethylaminoethyl)-[1,3]-dioxolane, Aceticacid(7R,9Z)-18-({[3-(dimethylamino)propyloxy]carbonyl}oxy)octacosa-9-en-7-yl, Aceticacid(20,23R)-2-methyl-9-[(9Z,12Z)-octadeca-9,12-dien-1-yl]-7-oxo-6,8,11-trioxa-2-azanonacosa-20-En-23-yl3-(dimethylamino)propylcarbonate(11Z,14Z)-1-{[(9Z,12R)-12-hydroxyoctadec-9-en-1-yl], 9Z,12Z)-3-((4,4-bis(octyloxy)butanoyl)oxy)-2-((((3-(diethylamino)propoxy)carbonyl)oxy)methyl)propyloctadeca-9,12-dienoate (Cationic Lipid A3), 9-(4-(Diisopropylamino)butyl)-7-hydroxyheptadecane-1,17-diyldioleate (CL4H8), 8-dimethylO′I,01-(2-(((1-methylpyrrolidine-3-carbonyl)oxy)methyl)propane-1,3-diyl)dioctanedioate, 7-Hydroxy7-(4-((1-methylpiperidine-4-carbonyl)oxy)butyl)tridecane-1,13-diylditetradecanoate (CL15C6), 7-Hydroxy7-(4-((1-methylpiperidine-4-carbonyl)oxy)butyl)tridecane-1,13-diyldipalmitate (CL15D6), 7-Hydroxy7-(4-((1-methylpiperidine-4-carbonyl)oxy)butyl)tridecane-1,13-diyldioleate (CL15H6), 7-Hydroxy7-(4-((1-methylpiperidine-4-carbonyl)oxy)butyl)tridecane-1,13-diyldidodecanoate (CL15B6), 7,10-dimethyl-13,16-dinonyl-6,11-dioxo-4-tetradecyl-4,7,10,13,16-pentaazapentacosyldecanoate, 7-(4-(Diisopropylamino)butyl)-7-hydroxytridecane-1,13-diylditetradecanoate (CL4C6), 7-(4-(Diisopropylamino)butyl)-7-hydroxytridecane-1,13-diyldipalmitate (CL4D6), 7-(4-(Diisopropylamino)butyl)-7-hydroxytridecane-1,13-diyldioleate (CL4H6), 6-((6,6-bis(octyloxy)hexanoyl)oxy)-4-(((3-(diethylamino)propoxy)carbonyl)oxy)hexyloctanoate, 5-((2-(4-(N-(2-(Dinonylamino)ethyl)-N-nonylglycyl)piperazin-oxoethyl)(nonyl)amino)pentylmethylcarbonate, 5-(((3-(dimethylamino)propoxy)carbonyl)oxy)-7-octylpentadecyloctanoate, 4-methyl-2,5-bis((9Z,12Z)-octadeca-9,12-dien-1-yloxy)benzyl4-(dimethylamino)butanoate, 4,4-bis(octyloxy)butyl5-(((3-(diethylamino)propoxy)carbonyl)oxy)heptadecanoate, 4,4-bis(octyloxy)butyl(3-(diethylamino)propyl)pentadecane-1,3-diyldicarbonate, 4,4′-((5-((dimethylamino)methyl)-1,3-phenylene)bis(oxy))bis(butanel,2-diyl)tetraoctanoate, 4-(dimethylamino)butylcarbonate(6Z,9Z,26Z,29Z)-pentatriaconta-6,9,26,29-tetraen-18-yl, 4-(dimethylamino)butyl4-methyl-2,5-bis((9Z,12Z)-octadeca-9,12-dienyloxy)benzylcarbonate, 4-(Didodecylamino)butan-1-ol (17-13), 4-(2-Aminoethyl)-Morpholino-Cholesterolhemisuccinat (MoChol), 4-(2-((2-(dinonylamino)ethyl)(nonyl)amino)acetamido)butylpentanoate, 4-((N-(2-(Dinonylamino)ethyl)-N-nonylglycyl)oxy)pentan-2-yldinonylglycinate, 4-((2-(Didodecylamino)ethyl)(dodecyl)amino)butan-1-ol (20-22), 3β-[N-(N′,N′-dimethylaminoethane)carbamoyl]cholesterol (DC-Chol), 3-Hydroxybutan-2-ylN-(2-(dinonylamino)ethyl)-N-nonyl, 3-Hydroxy-2,2-bis(((9Z)-tetradec-9-enoyloxy)methyl)propyl(9Z)-tetradec-9-enoate (Synthesis Example 1 (C)), 3-dimethylaminopropylcarbonate(9Z,12Z)-octacosa-19,22-dien-11-yl, 3-Dimethylamino-2-(Cholest-5-en-3β-oxypent-3-oxa-an-5-oxy)-1-(cis,cis-9,12-octadecadienoxy)propane (DEG-CLinDMA), 3-Dimethylamino-2-(Cholest-5-en-3B-oxybutan-4-oxy)-1-(cis,cis-9,12-octadecadienoxy)propane (CLinDMA), 3,5-bis((9Z,12Z)-octadeca-9,12-dien-1-yloxy)benzyl3-(dimethylamino)propanoate, 3,3-Di((9Z,12Z)-octadeca-9,12-dien-1-yl)cyclopentyl4-(dimethylamino)butanoate (21-4), 3,3-Di((9Z,12Z)-octadeca-9,12-dien-1-yl)cyclopentyl3-(dimethylamino)propanoate (21-3), 3,3-Di((9Z,12Z)-octadeca-9,12-dien-1-yl)cyclobutyl4-(dimethylamino)butanoate (21-2), 3-(N,N-Dioleylamino)-1,2-propanedio (DOAP), 3-(N,N-Dilinoleylamino)-1,2-propanediol (DLinAP), 3-(Dinonylamino)-I-(4-(3-((2-(dinonylamino)ethyl)(nonyl)amino)propanoyl)piperazin-1-yl)propan-1-one, 3-(Dinonylamino)-1-(4-(3-((2-(dinonylamino)ethyl)(nonyl)amino)propanoyl)piperazin-1-yl)propan-1-one, 3-(Dimethylamino)propyltriacontan-11-ylcarbonateTriacontan-11-ol, 3-(Dimethylamino)propyloctacosane-11-ylcarbonate, 3-(Dimethylamino)propylcarbonate(6Z,9Z,28Z,31Z)-heptatriconta-6,9,28,31-tetraen-19-yl, 3-(Dimethylamino)propylcarbonate(6Z,9Z,26Z,29Z)-pentatriacontour-6,9,26,29-tetraen-18-yl, 3-(dimethylamino)propyl4-methyl-2,5-bis((9Z,12Z)-octadeca-9,12-dien-1-yloxy)benzylcarbonate, 3-(Dimethylamino)propyl3,3-di((9Z,12Z)-octadeca-9,12-dien-1-yl)azetidine-1-carboxylate (CL-60), 3-(Dimethylamino)propane-1,2-diyl(9Z,9′Z,12Z,12′Z)-bis(octadeca-9,12-dienoate) (CL-14), 3-(Dimethylamino)-2,2-bis((((9Z,12Z)-octadeca-9,12-dien-1-yl)oxy)methyl)propan-1-ol (CL-65), 3-(Didodecylamino)propan-1-ol (17-12), 3-(didodecylamino)-N1,N1,4-tridodecyl-1-piperazineethanamine (KL10), 3-(Di((Z)-octadeca-9-en-1-yl)amino)propan-1-ol (CL-62), 3-(Di((9Z,12Z)-octadeca-9,12-dien-1-yl)amino)propan-1-o (CL-6), 3-(5-(bis(2-hydroxydodecyl)amino)pentan-2-yl)-6-(5-((2-hydroxydodecyl)(2-hydroxyundecyl)amino)pentan-2-yl)-1,4-dioxane-2,5-dione) (Target 24), 3-((tert-Butyl(dimethyl)silyl)oxy)-2,2-bis(((9Z)-tetradec-9-enoyloxy)methyl)propyl(9Z)-tetradec-9-enoate (Synthesis Example 1 (B)), 3-((dimethylamino)methyl)-5-(((8-(octanoyloxy)octanoyl)oxy)methyl)benzyl3-octylundecanoate, 3-((6Z,9Z,28Z,31Z)-heptatriaconta-6,9,28,31-tetraen-19-yloxy)-N,N-dimethylpropan-1-amine, 3-((6Z,9Z,28Z,31Z)-heptatriaconta-6,9,28,31-tetraen-19-yloxy)-N,N-dimethylpropan-1-amine, 3-((6-(dimethylamino)hexanoyl)oxy)-2,2-bis(((9Z)-tetradec-9-enoyloxy)methyl)propyl(9Z)-tetradec-9-enoate, 3-((5-(dimethylamino)pentanoyl)oxy)-2,2-bis(((9Z)-tetradec-9-enoyloxy)methyl)propyl(9Z,12Z)-octadec-9,12-dienoate, 3-((5-(dimethylamino)pentanoyl)oxy)-2,2-bis(((9Z)-tetradec-9-enoyloxy)methyl)propyl(9Z)-tetradec-9-enoate, 3-((5-(dimethylamino)pentanoyl)oxy)-2,2-bis(((9Z)-tetradec-9-enoyloxy)methyl)propyl(9Z)-octadec-9-enoate, 3-((5-(dimethylamino)pentanoyl)oxy)-2,2-bis(((9Z)-tetradec-9-enoyloxy)methyl)propyl(9Z)-hexadec-9-enoate, 3-((5-(Dimethylamino)pentanoyl)oxy)-2,2-bis(((3-pentyloctanoyl)oxy)methyl)propyl3-pentyloctanoate, 3-((5-(diethylamino)pentanoyl)oxy)pentadecyl6,6-bis(octyloxy)hexanoate, 3-((4,4-bis(octyloxy)butanoyl)oxy)-2-((tetradecanoyloxy)methyl)propyl1-methylpyrrolidine-3-carboxylate, 3-((4,4-bis(octyloxy)butanoyl)oxy)-2-((palmitoyloxy)methyl)propyl1-methylpyrrolidine-3-carboxylate, 3-((4,4-bis(octyloxy)butanoyl)oxy)-2-((dodecanoyloxy)methyl)propyl1-methylpyrrolidine-3-carboxylate, 3-((4,4-bis(octyloxy)butanoyl)oxy)-2-(((9Z,12Z)-octadeca-9,12-dienoyloxy)methyl)propyl4-methylmorpholine-2-carboxylate, 3-((4,4-bis(octyloxy)butanoyl)oxy)-2-(((9Z,12Z)-octadeca-9,12-dienoyloxy)methyl)propyl4-((diethylamino)methyl)benzoate, 3-((4,4-bis(octyloxy)butanoyl)oxy)-2-(((9Z,12Z)-octadeca-9,12-dienoyloxy)methyl)propyl3-((dimethylamino)methyl)benzoate, 3-((4,4-bis(octyloxy)butanoyl)oxy)-2-(((9Z,12Z)-octadeca-9,12-dienoyloxy)methyl)propyl1-methylpiperidine-4-carboxylate, 3-((4,4-bis(octyloxy)butanoyl)oxy)-2-(((9Z,12Z)-octadeca-9,12-dienoyloxy)methyl)propyl1-methylpiperidine-3-carboxylate, 3-((4,4-bis(octyloxy)butanoyl)oxy)-2-(((9Z,12Z)-octadeca-9,12-dienoyloxy)methyl)propyl1-isopropylpiperidine-4-carboxylate, 3-((4,4-bis(octyloxy)butanoyl)oxy)-2-(((9Z,12Z)-octadeca-9,12-dienoyloxy)methyl)propyl1,4-dimethylpiperidine-4-carboxylate, 3-((4,4-bis(octyloxy)butanoyl)oxy)-2-(((9Z,12Z)-octadeca-9,12-dienoyloxy)methyl)propyl1-(cyclopropylmethyl)piperidine-4-carboxylate, 3-((4,4-bis(octyloxy)butanoyl)oxy)-2-((((3-(diethylamino)propoxy)carbonyl)oxy)methyl)propyl(9Z,12Z)-octadeca-9,12-dienoate (Lipid A), 3-((4-(Dimethylamino)butanoyl)oxy)-2,2-bis(((9Z)-tetradec-9-enoyloxy)methyl)propyl(9Z)-tetradec-9-enoate (Synthesis Example 1 (D)), 3-((4-(dimethylamino)butanoyl)oxy)-2,2-bis(((9Z)-tetradec-9-enoyloxy)methyl)propyl(9Z)-hexadec-9-enoate, 3-((3-(I-(3-((2-(Dinonylamino)ethyl)(nonyl)amino)propanoyl)piperidin-4-yl)propyl)(nonyl)amino)propylhexanoate, 3-((3-(4-(3-((2-(Dinonylamino)ethyl)(nonyl)amino)propanoyl)piperazin-1-yl)-3-oxopropyl)(nonyl)amino)propylhexanoate, 3-((3-(4-(3-((2-(Dinonylamino)ethyl)(nonyl)amino)propanoyl)piperazin-1-yl)-3-oxopropyl)(nonyl)amino)propylhexanoate, 3-((3-(1-methylpiperidin-4-yl)propanoyl)oxy)pentadecyl6,6-bis(octyloxy)hexanoate, 3-((3-(1-(3-((2-(Dinonylamino)ethyl)(nonyl)amino)propanoyl)piperidin-4-yl)propyl)(nonyl)amino)propylhexanoate, 3-((2-(stearoyloxy)ethyl)amino)propanoicacid (MSAPA), 3-((2-(oleoyloxy)ethyl)amino)propanoicacid (MOAPA), 3-((2-(I-(N-(2-(Dinonylamino)ethyl)-N-nonylglycyl)pyrrolidin-3-yl)ethyl)(tetradecyl)amino)propyldecanoateter/-Butyl3-(2-((3-(decanoyloxy)propyl)(tetradecyl)amino)ethyl)pyrrolidine-I-carboxylate, 3-((2-(I-(N-(2-(Dinonylamino)ethyl)-N-nonylglycyl)piperidin-4-yl)ethyl)(tetradecyl)amino)propyldecanoate, 3-((2-(I-(N-(2-(Dinonylamino)ethyl)-N-nonylglycyl)piperidin-4-yl)ethyl)(nonyl)amino)propylhexanoate, 3-((2-(I-(N-(2-(Dinonylamino)ethyl)-N-nonylglycyl)piperidin-4-yl)ethyl)(nonyl)amino)-2-methylpropylhexanoate, 3-((2-(I-(N-(2-(Dinonylamino)ethyl)-N-nonylglycyl)piperidin-3-yl)ethyl)(tetradecyl)amino)propyldecanoate, 3-((2-(I-(N-(2-(Dinonylamino)ethyl)-N-nonylglycyl)piperidin-yl)ethyl) (nonyl)amino)propyl3-methylhexanoatefert-Butyl4-(2-((3-((3-methylhexanoyl)oxy)propyl)(nonyl)amino)ethyl)piperidine-I—, 3-((2-(Ditetradecylamino)ethyl)(dodecyl)amino)propan-1-ol (20-11), 3-((2-(Dinonylamino)ethyl)(nonyl)amino)-I-(4-(3-(dinonylamino)propyl)piperidin-1-yl)propan-1-one, 3-((2-(Dinonylamino)ethyl)(nonyl)amino)-1-(4-(3-(dinonylamino)propyl)piperidin-1-yl)propan-1-one, 3-((2-(Didodecylamino)ethyl)(dodecyl)amino)propan-1-ol (20-21), 3-((2-(4-(N-(2-(Dinonylamino)ethyl)-N-nonylglycyl)piperazin-oxoethyl)(nonyl)amino)propyl3-methylhexanoate, 3-((2-(4-(N-(2-(Dinonylamino)ethyl)-N-nonylglycyl)piperazin-oxoethyl)(nonyl)amino)-2-methylpropylhexanoate, 3-((2-(4-(N-(2-(Dinonylamino)ethyl)-N-nonylglycyl)piperazin-oxoethyl)(dodecyl)amino)propyloctanoatetot-Butyldodecylglycinate, 3-((2-(4-(N-(2-(Dinonylamino)ethyl)-N-nonylglycyl)piperazin-I-yl)-2-oxoethyl)(tetradecyl)amino)propyldecanoate, 3-((2-(4-(N-(2-(Dinonylamino)ethyl)-N-nonylglycyl)piperazin-I-yl)-2-oxoethyl)(tetradecyl)amino)propyl(Z)-dec-3-enoate(Z)-Dec-3-en-I-ol, 3-((2-(4-(N-(2-(Dinonylamino)ethyl)-N-nonylglycyl)piperazin--yl)-2-oxoethyl)(nonyl)amino)propylhexanoate, 3-((2-(4-(N-(2-(Dinonylamino)ethyl)-N-nonylglycyl)piperazin-1-yl)-2-oxoethyl)(nonyl)amino)propylhexanoate, 3-((2-(1-(N-(2-(Dinonylamino)ethyl)-N-nonylglycyl)piperidin-4-yl)ethyl)(nonyl)amino)propylhexanoate, 3-((2-(((Z)-octadeca-9-en-1-yl)oxy)ethyl)((9Z,12Z)-octadeca-9,12-dien-1-yl)amino)propane-1-All (CL-54), 3-((2-(((9Z,12Z,15Z)-octadeca-9,12,15-trienoyl)oxy)ethvnamino)propanoicacid (MLLAPA), 3-((2-(((9Z,12Z)-octadeca-9,12-dienoyl)oxy)ethyl)amino)propanoicacid (MLAPA), 3-((1,3-bis(stearoyloxy)propan-2-yl)amino)propanoicacid (DSAPA), 3-((1,3-bis(stearoyloxy)-2-((stearoyloxy)methyl)propan-2-yl)amino)propanoicacid (TSAPA), 3-((1,3-bis(oleoyloxy)propan-2-yl)amino)propanoicacid (DOAPA), 3-((1,3-bis(((Z)-octadec-9-enoyl)oxy)-2-((((Z)-octadec-9-enoyl)oxy)methyl)propan-2-yl)amino)propanoicacid (TOAPA), 3-((1,3-bis(((9Z.12Z.15Z)-octadeca-9.12.15-trienoyl)oxy)-2-((((9Z.12Z.15E)-octadeca-9,12,15-trienoyl)oxy)methyl)propan-2-yl)amino)propanoicacid (TLLAPA), 3-((1,3-bis(((9Z,12Z.15Z)-octadeca-9,12,15-trienoyl)oxy)propan-2-yl)amino)propanoicacid (DLLAPA), 3-((1,3-bis(((9Z,12Z)-octadeca-9,12-dienoyl)oxy)propan-2-yl)amino)propanoicacid (DLAPA), 3-((1,3-bis(((9Z,12Z)-octadeca-9,12-dienoyl)oxy)-2-((((9Z,12Z)-octadeca-9,12-dienoyl)oxy)methyl)propan-2-yl)amino)propanoicacid (TLAPA), 3-(((4-(diethylamino)butoxy)carbonyl)oxy)pentadecyl6,6-bis(octyloxy)hexanote, 3-(((3-piperidin-1-yl)propoxy)carbonyl)oxy)pentadecyl6.6-bis(octyloxy)hexanoate, 3-(((3-morpholinoproproxy)carbonyl)oxy)pentadecyl6,6-bis(octyloxy)hexanoate, 3-(((3-dimethylamino)propoxy)carbonyl)oxy)pentadecyl3octylundecanoate, 3-(((3-(piperazin-1-yl)propoxy)carbonyl)oxy)pentadecyl6,6-bis(octyloxy)hexanoate, 3-(((3-(ethyl(methyl)amino)propoxy)carbonyl)oxy)pentadecyl8,8-bis((2-propylpentyl)oxy)octanoate, 3-(((3-(ethyl(methyl)amino)propoxy)carbonyl)oxy)pentadecyl4,4-bis((2-propylpentyl)oxy)butanoate, 3-(((3-(dimethylamino)propoxy)carbonyl)oxy)pentadecyl9-pentyltetradecanoate, 3-(((3-(dimethylamino)propoxy)carbonyl)oxy)pentadecyl8,8-bis((2-propylpentyl)oxy)octanoate, 3-(((3-(dimethylamino)propoxy)carbonyl)oxy)pentadecyl7-hexyltridec-6-enoate, 3-(((3-(dimethylamino)propoxy)carbonyl)oxy)pentadecyl6,6-bis((3-ethylpentyl)oxy)hexanote, 3-(((3-(dimethylamino)propoxy)carbonyl)oxy)pentadecyl6,6-bis((2-propylpentyl)oxy)hexanoate, 3-(((3-(dimethylamino)propoxy)carbonyl)oxy)pentadecyl5-heptyldodecanoate, 3-(((3-(dimethylamino)propoxy)carbonyl)oxy)pentadecyl4,4-bis(octyloxy)butanoate, 3-(((3-(dimethylamino)propoxy)carbonyl)oxy)pentadecyl3-octylundec-2-enoate, 3-(((3-(dimethylamino)propoxy)carbonyl)oxy)pendadecyl4,4-bis((2-propylpentyl)oxy)butanoate, 3-(((3-(dimethylamino)propoxy)carbonyl)oxy)-5-octyltridecyldecanoate, 3-(((3-(dimethylamino)propoxy)carbonyl)oxy)-13-hydroxytridecyl9-pentyltetradecanoate, 3-(((3-(dimethylamino)propoxy)carbonyl)oxy)-13-hydroxytridecyl5-heptyldodecanoate, 3-(((3-(dimethylamino)propoxy)carbonyl)oxy)-13-(octanoyloxy)tridecyl9-pentyltetradecanoate, 3-(((3-(dimethylamino)propoxy)carbonyl)oxy)-13-(octanoyloxy)tridecyl7-hexyltridecanoate, 3-(((3-(dimethylamino)propoxy)carbonyl)oxy)-13-(octanoyloxy)tridecyl5-heptyldodecanoate, 3-(((3-(dimethylamino)propoxy)carbonyl)oxy)-13-(octanoyloxy)tridecyl3-octylundecanoate (Lipid D), 3-(((3-(diethylamino)propoxy)carbonyl)oxy)undecyl6,6-bis(octyloxy)hexanoate, 3-(((3-(diethylamino)propoxy)carbonyl)oxy)tridecyl6,6-bis(octyloxy)hexanoate, 3-(((3-(diethylamino)propoxy)carbonyl)oxy)pentadecyl8,8-dibutoxyoctanoate, 3-(((3-(diethylamino)propoxy)carbonyl)oxy)pentadecyl8,8-bis(octyloxy)octanoate, 3-(((3-(diethylamino)propoxy)carbonyl)oxy)pentadecyl8,8-bis((2-propylpentyl)oxy)octanoate, 3-(((3-(diethylamino)propoxy)carbonyl)oxy)pentadecyl6,6-bis(octyloxy)hexanoate, 3-(((3-(diethylamino)propoxy)carbonyl)oxy)pentadecyl6,6-bis(hexyloxy)hexanoate, 3-(((3-(diethylamino)propoxy)carbonyl)oxy)pentadecyl6,6-bis((2-propylpentyl)oxy)hexanoate, 3-(((3-(diethylamino)propoxy)carbonyl)oxy)pentadecyl6,6-bis((2-ethylhexyl)oxy)hexanoate, 3-(((3-(diethylamino)propoxy)carbonyl)oxy)pentadecyl5-(4,6-diheptyl-1,3-dioxan-2-yl)pentanoate, 3-(((3-(diethylamino)propoxy)carbonyl)oxy)pentadecyl4,4-bis(octyloxy)butanoate, 3-(((3-(diethylamino)propoxy)carbonyl)oxy)pentadecyl4,4-bis((2-propylpentyl)oxy)butanoate, 3-(((3-(diethylamino)propoxy)carbonyl)oxy)pentadecyl4,4-bis((2-ethylhexyl)oxy)butanoate, 3-(((3-(4-methylpiperazin-1-yl)propoxy)carbonyl)oxy)pentadecyl6,6-bis(octyloxy)hexanoate, 3-(((2-(diethylamino)ethoxy)carbonyl)oxy)pentadecyl6,6-bis(octyloxy)hexanoate, 3-((((1-methylpiperidin-4-yl)methoxy)carbonyl)oxy)pentadecyl6,6-bis(octyloxy)hexanoate, 2-amino-N,N-dihexadecyl-3-(1H-imidazol-5-yl)propanamide, 2-{4-[(3p)-cholest-5-en-3-yloxy]butoxy}-iV?N-dimethyl-3-[(9Z,12Z)-octadeca-9-12-dien-1-yloxy]propan-1-amine (Butyl-CLinDMA), 2-[5′-(cholest-5-en-3-oxy)-3′-oxapentoxy)-3-dimethy-1-(cis,cis-9′,12′-octadecadienoxy)propane (CpLinDMA), 2,5-bis((9Z,12Z)-octadeca-9,12-dienyloxy)benzyl3-(dimethylamino)propylcarbonate, 2,5-bis((9Z,12Z)-octadeca-9,12-dien-1-yloxy)benzyl4-(dimethylamino)butanoate, 2,5-bis((9Z,12Z)-octadeca-9,12-dien-1-yloxy)benzyl3-(dimethylamino)propanoate, 2,4-bis((9Z,12Z)-octadeca-9,12-dienyloxy)benzyl4-(dimethylamino)butanoate, 2,3-distearyloxypropylamine, 2,3-dioleyloxypropylamine, 2,3-dioleyloxy-N-[2(spermine-carboxamido)ethyl]-N,N-dimethyl-1-propanaminiumtrifluoroacetate (DOSPA), 2,3-dilauryloxypropylamine, 2,3-bis(dodecylthio)propyl(2-(dimethylamino)ethyl)carbamate (DPDEC), 2,2-Dillinoleyl-4-dimethylaminoethyl-[1,3]-dioxolane (XTC), 2,2-Dilinoleyl-4-dimethylaminomethyl-[1,3]-dioxolane (DLin-K-DMA), 2,2-Dilinoleyl-4-dimethylaminoethyl-[1,3]-dioxolane (Lipid A2), 2,2-dilinoleyl-4-(2-dimethylaminoethyl)-[1,3]-dioxolane (DLin-K-C2-DMA), 2,2-bis(heptyloxy)ethyl3-((3-ethyl-10-((9Z,12Z)-octadeca-9,12-dien-1-yl)-8,15-dioxo-7,9,14-trioxa-3-azaheptadecan-17-yl)disulfanyl)propanoate, 2,2′-(tert-butoxycarbonylazanediyl)bis(ethane-2,1-diyl)ditetradecanoate (HEDC-BOC-TN), 2-(Nonyl((9Z,12Z)-octadeca-9,12-dien-1-yl)amino)ethan-1-ol (17-3), 2-(I-(N-(2-(Dinonylamino)ethyl)-N-nonylglycyl)pyrrolidin-3-yl)ethyldinonylglycinate, 2-(I-(N-(2-(Dinonylamino)ethyl)-N-nonylglycyl)piperidin-4-yl)ethyldinonylglycinatefert-Butyl4-(2-((dinonylglycyl)oxy)ethyl)piperidine-1-carboxylate, 2-(Hexyl((9Z,12Z)-octadeca-9,12-dien-1-yl)amino)ethan-1-ol (17-2), 2-(Dodecyl((9Z,12Z)-octadeca-9,12-dien-1-yl)amino)ethan-1-ol (17-4), 2-(Ditetradecylamino)ethan-1-ol (17-7), 2-(dinonylamino)-N-(4-(2-((2-(dinonylamino)ethyl)(nonyl)amino)-N-methylacetamido)butyl)-N-methylacetamide, 2-(dinonylamino)-N-(3-(2-((2-(dinonylamino)ethyl)(nonyl)amino)-N-methylacetamido)propyl)-N-methylacetamide, 2-(Dinonylamino)-N-(2-(2-((2-(dinonylamino)ethyl)(nonyl)amino)-N-methylacetamido)ethyl)-N-methylacetamide2-(Dinonylamino)-N-methyl-N-(2-(methylamino)ethyl)acetamide, 2-(dinonylamino)-N-(2-(2-((2-(dinonylamino)ethyl)(nonyl)amino)-N-ethylacetamido)ethyl)-N-ethylacetamide, 2-(dinonylamino)-N-(2-(2-((2-(dinonylamino)ethyl)(nonyl)amino)acetamido)ethyl)acetamide, 2-(Dinonylamino)-1-(5-(N-(2-(dinonylamino)ethyl)-N-nonylglycyl)-2,5-diazabicyclo[2.2.1]heptan-2-yl)ethan-1-one, 2-(Dinonylamino)-I-(4-(N-(2-(dinonylamino)ethyl)-N-nonylglycyl)piperazin-I-yl)ethan-1-one, 2-(Dinonylamino)-1-(4-(N-(2-(dinonylamino)ethyl)-N-dodecylglycyl)piperazin-1-yl)ethan-1-one, 2-(Dinonylamino)-1-(4-(2-((2-(dinonylamino)ethyl)(nonyl)amino)ethyl)piperidin-1-yl)ethan-1-one, 2-(Dinonylamino)ethan-1-ol (17-10), 2-(Dinonylamino)-1-(5-(N-(2-(dinonylamino)ethyl)-N-nonylglycyl)-2,5-diazabicyclo[2.2.1]heptan-2-yl)ethan-1-one, 2-(Dinonylamino)-1-(4-(2-((2-(dinonylamino)ethyl)(nonyl)amino)ethyl)piperidin-1-yl)ethan-1-one, 2-(Dimethylamino)-N-(2-(((Z)-octadeca-9-en-1-yl)oxy)ethyl)-N-((9Z,12Z)-octadeca-9,12-diene-1-yl)acetamide (CL-53), 2-(Dimethylamino)-N-((6Z,9Z,28Z,31Z)-Heptatriconta-6,9,28,31-tetraen-19-yl)acetamide (CL-13), 2-(Didodecylamino)-N-dodecyl-N-(2-(piperazin-I-yl)ethyl)acetamide, 2-(Didodecylamino)-N-(2-(4-(2-(didodecylamino)ethyl)piperazin--yl)ethyl)-N-dodecylacetamide, 2-(Didodecylamino)-I-(4-(N-(2-(didodecylamino)ethyl)-N-dodecylglycyl)piperazin-1-yl)ethan-1-one, 2-(Didodecylamino)-I-(4-(N-(2-(didodecylamino)ethyl)-N-dodecylglycyl)piperazin-1-yl)ethan-1-one, 2-(Didodecylamino)-I-(4-(2-((2-(didodecylamino)ethyl)(dodecyl)amino)ethyl)piperazin--yl)ethan-1-one, 2-(Didodecylamino)-I-(4-(2-((2-(didodecylamino)ethyl)(dodecyl)amino)ethyl)piperazin-1-yl)ethan-1-one, 2-(Didodecylamino)ethan-1-ol (17-11), 2-(Didodecylamino)-1-(4-(N-(2-(dinonylamino)ethyl)-N-nonylglycyl)piperazin-1-yl)ethan-1-one, 2-(Didodecylamino)-1-(4-(N-(2-(dinonylamino)ethyl)-N-nonylglycyl)piperazin-1-yl)ethan-1-, 2-(Didodecylamino)-1-(4-(N-(2-(dinonylamino)ethyl)-N-dodecylglycyl)piperazin-1-yl)ethan-1-one, 2-(Didodecylamino)-1-(4-(N-(2-(didodecylamino)ethyl)-N-nonylglycyl)piperazin-1-yl)ethan-1-one, 2-(Di((Z)-octadeca-9-en-1-yl)amino)ethane-1-ol (CL-61), 2-(Di((Z)-octadec-9-en-1-yl)amino)ethan-1-ol (17-8), 2-(bis(2-(tetradecanoyloxy)ethyl)amino)-N-(2-hydroxyethyl)-N,N-dimethyl-2-oxoethan-aminiumbromide (HEDC), 2-(9-dodecyl-2-methyl-7,13-dioxo-6,8,12-trioxa-2-azanonadecan-19-yl)propane-1,3-diyldioctanoate, 2-(5-oxo-5-((4-(((S)-pyrrolidine-2-carbonyl)oxy)hexadecyl)oxy)pentyl)propane-1,3-diyldioctanoate, 2-(5-oxo-5-((3-(((3-(piperidin-1-yl)propoxy)carbonyl)oxy)pentadecyl)oxy)pentyl)propane-1,3-diyldioctanoate, 2-(5-(3-((1-methylpyrrolidine-3-carbonyl)oxy)-2-((tetradecanoyloxy)methyl)propoxy)-5-oxopentyl)propane-1,3-diyldioctanoate, 2-(5-(3-((1-methylpyrrolidine-3-carbonyl)oxy)-2-((palmitoyloxy)methyl)propoxy)-5-oxopentyl)propane-1,3-diyldioctanoate, 2-(5-((4-((1,4-dimethylpiperidine-4-carbonyl)oxy)hexadecyl)oxy)-5-oxopentyl)propane-1,3-diyldioctanoate, 2-(5-((4-((1,3-dimethylpyrrolidine-3-carbonyl)oxy)hexadecyl)oxy)-5-oxopentyl)propane-1,3-diyldioctanoate, 2-(5-((4-((((1-methylpiperidin-4-yl)oxy)carbonyl)oxy)hexadecyl)oxy)-5-oxopentyl)propane-1,3-diyldioctanoate, 2-(5-((4-((((1-ethylpiperidin-3-yl)methoxy)carbonyl)oxy)hexadecyl)oxy)-5-oxopentyl)propane-1,3-diyldioctanoate, 2-(5-((4-(((((S)-1-methylpyrrolidin-3-yl)oxy)carbonyl)oxy)hexadecyl)oxy)-5-oxopentyl)propane-1,3-diyldioctanoate, 2-(5-((4-(((((R)-1-methylpyrrolidin-3-yl)oxy)carbonyl)oxy)hexadecyl)oxy)-5-oxopentyl)propane-1,3-diyldioctanoate, 2-(3,3-di((9Z,12Z)-octadeca-9,12-dien-1-yl)azetidin-1-yl)propan-1-ol (CL-59), 2-(3,3-di((9Z,12Z)-octadeca-9,12-dien-1-yl)azetidin-1-yl)ethan-1-ol (CL-58), 2-(3-(4-(5-((dimethylamino)methyl)-2-methyl-3-((9Z,12Z)-octadeca9,12-dien-1-yloxy)phenoxy)butoxy)-3-oxopropyl)propane-1,3-diyldihexanoate, 2-(12-dodecyl-3-ethyl-8,14-dioxo-7,9,13-trioxa-3-azaoctadecan-18-yl)propane-1,3-diyldioctanoate, 2-(11-dodecyl-3-ethyl-9,15-dioxo-8,10,14-trioxa-3-azanonadecan-19-yl)propane-1,3-diyldioctanoate, 2-(10-dodecyl-3-ethyl-8,15-dioxo-7,9,14-trioxa-3-azanonadecan-19-yl)propane-1,3-diyldioctanoate, 2-(10-dodecyl-3-ethyl-8,14-dioxo-7,9,13-trioxa-3-azaoctadecan-18-yl)propane-1,3-diyldioctanoate, 2-(10-dodecyl-3-ethyl-8,14-dioxo-7,9,13-trioxa-3-azanonadecan-19-yl)propane-1,3-diyldioctanoate, 2-(10-dodecyl-3-ethyl-8,14-dioxo-7,9,13-trioxa-3-azaicosan-20-yl)propane-1,3-diyldioctanoate, 2-(10-dodecyl-3-ethyl-8,14-dioxo-7,9,13-trioxa-3-azahexadecan-16-yl)propane-1,3-diyldioctanoate, 2-({8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA), 2-((N-(2-(Dinonylamino)ethyl)-N-nonylglycyl)oxy)ethyldinonylglycinate2-Hydroxyethyldinonylglycinate, 2-((decanoyloxy)methyl)-2-(((4-(dimethylamino)butanoyl)oxy)methyl)propane-1,3-diyl(9Z,9′Z)bis-tetradec-9-enoate, 2-((4-(((3-(ethyl(methyl)amino)propoxy)carbonyl)oxy)hexadecanoyl)oxy)propane-1,3-diylditetradecanoate, 2-((4-(((3-(ethyl(methyl)amino)propoxy)carbonyl)oxy)hexadecanoyl)oxy)propane-1,3-diyldioctanoate, 2-((4-(((3-(ethyl(methyl)amino)propoxy)carbonyl)oxy)hexadecanoyl)oxy)propane-1,3-diyldidodecanoate, 2-((4-(((3-(ethyl(methyl)amino)propoxy)carbonyl)oxy)hexadecanoyl)oxy)propane-1,3-diylbis(decanoate), 2-((4-(((3-(dimethylamino)propoxy)carbonyl)oxy)hexadecanoyl)oxy)propane-1,3-diyl9Z,9′Z,12Z,12′Z)-bis(octadeca-9,12-dienoate) (Lipid C), 2-((4-(((3-(dimethylamino)propoxy)carbonyl)oxy)hexadecanoyl)oxy)propane-1,3-diylditetradecanoate, 2-((4-(((3-(dimethylamino)propoxy)carbonyl)oxy)hexadecanoyl)oxy)propane-1,3-diyldidodecanoate, 2-((4-(((3-(diethylamino)propoxy)carbonyl)oxy)hexadecanoyl)oxy)propane-1,3-diylditetradecanoate, 2-((4-(((3-(diethylamino)propoxy)carbonyl)oxy)hexadecanoyl)oxy)propane-1,3-diyldidodecanoate, 2-((4-(((3-(diethylamino)propoxy)carbonyl)oxy)hexadecanoyl)oxy)propane-1,3-diylbis(decanoate), 2-((2-(Ditetradecylamino)ethyl)(tetradecyl)amino)ethan-1-ol (20-12), 2-((2-(dinonylamino)ethyl)(nonyl)armno)-N-tetradecylacetamide, 2-((2-(Dinonylamino)ethyl)(nonyl)amino)-I-(4-(ditetradecylglycyl)piperazin-1-yl)ethan-1-one, 2-((2-(Dinonylamino)ethyl)(nonyl)amino)-I-(4-(2-(dinonylamino)ethyl)piperidin-1-yl)ethan-I-one, 2-((2-(Dinonylamino)ethyl)(nonyl)amino)-I-(3-(2-(dinonylamino)ethyl)pyrrolidin-1-yl)ethan-1-one, 2-((2-(Dinonylamino)ethyl)(nonyl)amino)-I-(3-(2-(dinonylamino)ethyl)piperidin-I-yl)ethan-I-one, 2-((2-(Dinonylamino)ethyl)(nonyl)amino)-I-(3-((dinonylamino)methyl)pyrrolidin-1-yl)ethan-1-one, 2-((2-(Dinonylamino)ethyl)(nonyl)amino)ethyltetradecanoate, 2-((2-(Dinonylamino)ethyl)(nonyl)amino)ethylnonanoate, 2-((2-(Dinonylamino)ethyl)(nonyl)amino)ethyl1-(dinonylglycyl)piperidine-4-carboxylate, 2-((2-(Dinonylamino)ethyl)(nonyl)amino)ethan-1-ol (20-16), 2-((2-(Dinonylamino)ethyl)(nonyl)amino)-1-(4-(ditetradecylglycyl)piperazin-1-yl)ethan-1-one, 2-((2-(Dinonylamino)ethyl)(nonyl)amino)-1-(4-(2-(dinonylamino)ethyl)piperidin-1-yl)ethan-1-one, 2-((2-(Dinonylamino)ethyl)(nonyl)amino)-1-(3-(2-(dinonylamino)ethyl)pyrrolidin-1-yl)ethan-1-one, 2-((2-(Dinonylamino)ethyl)(nonyl)amino)-1-(3-(2-(dinonylamino)ethyl)piperidin-1-yl)ethan-1-one, 2-((2-(Dinonylamino)ethyl)(nonyl)amino)-1-(3-((dinonylamino)methyl)pyrrolidin-1-yl)ethan-1-one, 2-((2-(Dinonylamino)ethyl)(dodecyl)amino)ethan-1-ol (20-18), 2-((2-(Didodecylamino)ethyl)amino)ethan-1-ol (20-19), 2-((2-(Didodecylamino)ethyl)(tetradecyl)amino)ethan-1-ol (20-24), 2-((2-(Didodecylamino)ethyl)(nonyl)amino)ethan-1-ol (20-17), 2-((2-(Didodecylamino)ethyl)(nonyl)amino)-1-(4-(dinonylglycyl)piperazin-1-yl)ethan-1-oneStep1:MethylN-(2-(didodecylamino)ethyl)-N-nonylglycinate, 2-((2-(Didodecylamino)ethyl)(nonyl)amino)-1-(4-(dinonylglycyl)piperazin-1-yl)ethan-1-one, 2-((2-(Didodecylamino)ethyl)(hexyl)amino)ethan-1-ol (20-15), 2-((2-(Didodecylamino)ethyl)(dodecyl)amino)-1-(5-(dinonylglycyl)-2,5-diazabicyclo[2.2.1]heptan-2-yl)ethan-1-one3, 2-((2-(Didodecylamino)ethyl)(dodecyl)amino)-I-(4-(2-(didodecylamino)ethyl)piperazin--yl)ethan-1-one, 2-((2-(Didodecylamino)ethyl)(dodecyl)amino)-I-(4-(2-(didodecylamino)ethyl)piperazin-1-yl)ethan-1-one, 2-((2-(Didodecylamino)ethyl)(dodecyl)amino)ethan-1-ol (20-20), 2-((2-(Didodecylamino)ethyl)(dodecyl)amino)-1-(5-(dinonylglycyl)-2,5-diazabicyclo[2.2.1]heptan-2-yl)ethan-1-one3, 2-((2-(Didodecylamino)ethyl)(dodecyl)amino)-1-(4-(dinonylglycyl)piperazin-1-yl)ethan-1-one, 2-((2-(Didodecylamino)ethyl)((9Z,12Z)-octadeca-9,12-dien-1-yl)amino)ethan-1-ol (20-25), 2-((2-(di((Z)-non-3-en-1-yl)amino)ethyl)((Z)-non-3-en-1-yl)amino)-N-(2-(2-(dinonylamino)-N-methylacetamido)ethyl)-N-methylacetamide, 2-((2-(Di((Z)-non-3-en-1-yl)amino)ethyl)((Z)-non-3-en-1-yl)amino)-I-(4-(dinonylglycyl)piperazin-1-yl)ethan-1-one(Z)-1-Bromonon-4-ene, 2-((2-(Di((9Z,12Z)-octadeca-9,12-dien-1-yl)amino)ethyl)(dodecyl)amino)ethan-1-ol (20-13), 2-((2-(Di((9Z,12Z)-octadeca-9,12-dien-1-yl)amino)ethyl)((9Z,12Z)-octadeca-9,12-dien-1-yl)amino)ethan-1-ol (20-14), 2-((2-(((3-(diethylamino)propoxy)carbonyl)oxy)tetradecanoyl)oxy)propane-1,3-diyldioctanoate, 2-(((tert-Butyldimethylsilyl)oxy)methyl)-2-(hydroxymethyl)propane-1,3-diol (Synthesis Example 1 (A)), 2-(((N,N-dimethyl-β-alanyl)oxy]methyl}-2-[(octanoyloxy)methyl)propane-1,3-diyl(9Z,9′Z)bis-tetradec-9-enoate, 2-(((9Z,12Z)-Octadeca-9,12-dien-1-yl)(tetradecyl)amino)ethan-1-ol (17-5), 2-(((9Z,12Z)-Octadeca-9,12-dien-1-yl)(octadecyl)amino)ethan-1-ol (17-6), 2-(((4-(dimethylamino)butanoyl)oxy)methyl)-2-((octanoyloxy)methyl)propane-1,3-diyl(9Z,9′Z)bis-tetradec-9-enoate, 2-(((4-(dimethylamino)butanoyl)oxy)methyl)-2-((dodecanoyloxy)methyl)propane-1,3-diyl(9Z,9′Z)bis-tetradec-9-enoate, 2-(((3-(dimethylamino)propanoyl)oxy)methyl)propane-1,3-diylbis(4,4-bis(octyloxy)butanoate), 2-(((3-(diethylamino)propoxy)carbonyl)oxy)tetradecyl4,4-bis((2-ethylhexyl)oxy)butanoate, 2-(((1-methylpyrrolidine-3-carbonyl)oxy)methyl)propane-1,3-diylbis(6,6-bis(octyloxy)hexanoate), 2-(((1-methylpyrrolidine-3-carbonyl)oxy)methyl)propane-1,3-diylbis(4,4-bis(octyloxy)buta, 2-(((13Z,16Z)-4-(((3-(dimethylamino)propoxy)carbonyl)oxy)docosa-13,16-dienoyl)oxy)propane-1,3-diyldioctanoate, 2-(((13Z,16Z)-4-(((3-(diethylamino)propoxy)carbonyl)oxy)docosa-13,16-dienoyl)oxy)propane-1,3-diyldioctanoate, 2-(((1,3-dimethylpyrrolidine-3-carbonyl)oxy)methyl)propane-1,3-diylbis(8-(octanoyloxy)octanoate), 2-(((1,3-dimethylpyrrolidine-3-carbonyl)oxy)methyl)propane-1,3-diylbis(4,4-bis(octyloxy)butanoate), 2-((((3-(dimethylamino)propoxy)carbonyl)oxy)methyl)propane-1,3-diylbis(2-heptylundecanoate), 1-Methyl-3,3-di((9Z,12Z)-octadeca-9,12-dien-1-yl)azetidine (CL-8), 1-Methyl-3,3-bis(2-(((9Z,12Z)-octadeca-9,12-dien-1-yl)oxy)propyl)azetidine (CL-57), 1-Methyl-3,3-bis(2-(((9Z,12Z)-octadeca-9,12-dien-1-yl)oxy)ethyl)azetidine (CL-56), 1-Methyl-3,3-bis((((9Z,12Z)-octadeca-9,12-dien-1-yl)oxy)methyl)azetidine (CL-55), 1-methyl18-[(2Z)-non-2-en-1-yl]9-{[4-(dimethylamino)butanoyl]oxy}octadecanedioate, 1-Linoleoyl-2-linoleyloxy-3-dimethylaminopropane (DLin-2-DMAP), 14,25-ditridecyl-15,18,21,24-tetraaza-octatriacontane (KL25), 11-(4-(Diisopropylamino)butyl)-11-hydroxyhenicosane-1,21-diyldioleate (CL4H110), 1-[2-(hexadecanoyloxy)ethyl]-2-pentadecyl-3-(2-hydroxyethyl)imidazoliniumchloride (DPTIM), 1-[2-(9(Z)-octadecenoyloxy)ethyl]-2-(8(Z)-heptadecenyl-3-(2-hydroxyethyl)imidazoliniumchloride (DOTIM), 1-[(1S,2R)-2-hexylcyclopropyl]-N,N-dimethylnonadecan-10-amine, 1-[(1S,2R)-2-decylcyclopropyl]-N,N-dimethylpentadecan-6-amine, 1-[(1R,2S)-2-heptylcyclopropyl]-N,N-dimethyloctadecan-9-amine, 1-[(11Z,14Z)-1-nonylicosa-11,14-dien-1-yl]pyrrolidine, 1.2Dilinoleyloxy3-(3′-hydroxypiperidino)-propylamine (DLinPip-30H), 1,N19-bis((16E,18E,20E,22E)-17,21-dimethyl-15-oxo-23-(2,6,6-trimethylcyclohex-1-en-1-yl)-4,7,10-trioxa-14-azatricosa-16,18,20,22-tetraen-1-yl)-4,7,10,13,16-pentaoxanonadecane-1,19-diamide (VA-PEG-VA), 1,6-DileoylTriethylenetetramide (dio-TETA), 1,3-dioleoyloxy-2-(6-carboxy-spermyl)-propylamide (DOSPER), 1,3-Bis-(1,2-bis-tetradecyloxy-propyl-3-dimethylethoxyammoniumbromide)-propan-2-ol ((R)-PLC-2), 1,2-N,N′-dioleylcarbamyl-3-dimethylaminopropane (DOcarbDAP), 1,2-N,N′-Dilinoleylcarbamyl-3-dimethylaminopropane (DLincarbDAP), 1,2-di-γ-Iinolenyloxy-N,N-dimethylaminopropane (γ-DLenDMA), 1,2-disteryloxypropyl-3-dimethyl-hydroxyethylammoniumbromide (DSRIE), 1,2-distearloxy-N,N-dimethylaminopropane (DSDMA1), 1,2-Diphvtanyloxy-W.N-dimemyl)-butyl-4-amme (DPan-C2-DMA), 1,2-dipalmityloxypropyl-3-dimethyl-hydroxyethylammoniumbromide (DPRIE), 1,2-dipalmitoyl-3-trimethylammoniumpropane (DPTAP), 1,2-dioleyloxypropyl-3-dimetyl-hydroxypropylammoniumbromide (DORIE-HP), 1,2-dioleyloxypropyl-3-dimethyl-hydroxypentylammoniumbromide (DORIE-Hpe), 1,2-dioleyloxypropyl-3-dimethyl-hydroxyethylammoniumbromide (DORIE), 1,2-dioleyloxypropyl-3-dimethyl-hydroxybutylammoniumbromide (DORIE-HB), 1,2-dioleoyl-sn-glycero-3-ethylphosphocholine (EDOPC), 1,2-Dioleoylcarbamyl-3-Dimethylammonium-propane (DOCDAP), 1,2-dioleoyl-3-trimethylammoniumpropane (DOTAP1), 1,2-Dioleoyl-3-N-pyrrolidine-propane (DOP5P), 1,2-Dioleoyl-3-N-pyrridinium-propane,bromidesalt (DOP6P), 1,2-Dioleoyl-3-N-morpholine-propane (MoDO), 1,2-Dioleoyl-3-methyl-(methoxycarbonyl-ethyl)ammonium-Propane (DOMCAP), 1,2-dioleoyl-3-dimethyl-hydroxyethylammoniumbromide (DORI), 1,2-Dioleoyl-3-Dimethylammonium-propane (DODAP), 1,2-Dimyristyloxypropyl-3-dimethyl-hydroxyethylammoniumbromide (DMRIE1), 1,2-Dilinoleyoxy-3-morpholinopropane (DLin-MA), 1,2-Dilinoleyoxy-3-(dimethylamino)acetoxy propane (DLin-DAC), 1,2-Dilinoleylthio-3-dimethylaminopropane (DLin-S-DMA), 1,2-DiLinoleyloxy-N,N-dimethylaminopropane (DLinDMA), 1,2-Dilinoleyloxy-3-trimethylaminopropanechloridesalt (DLin-TMA.Cl), 1,2-Dilinoleyloxy-3-hvdroxypropane (DLinPO), 1,2-dilinoleyloxy-3-dimethylaminopropane (DLinDMA 1), 1,2-Dilinoleyloxy-3-(N-methyl piperazino)propane (DLin-MPZ), 1,2 Dilinoleyloxy3-(N, N dimethyl D-propylamme (DLmDEA), 1,2Dilinoleyloxy3-(4′-hvdroxypiperidino)-propylamine (DLinPip-40H), 1,2-Dilinoleyloxo-3-(2-N,N-dimethylamino) ethoxy propane (DLin-EG-DMA), 1,2-Dilinoleylcarbamoyloxy-3-dimethylaminopropane (DLin-C-DAP), 1,2-Dilinoleoylcarbamyl-3-dimethylaminopropane (DLinCDAP), 1,2-Dilinoleoyl-3-trimethylaminopropanechloridesalt (DLin-TAP. C1), 1,2-Dilinoleoyl-3-dimethylaminopropane (DLinDAP), 1,2-dilinolenyloxy-3-dimethylaminopropane (DLenDMA), 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 1,1′-(2-(4-(2-((2-(bis(2hydroxydodecyl)amino)ethyl)(2-hydroxydodecyl)amino)ethyl)piperazin-1-yl)ethylazanediyl)didodecan-2-ol (Tech G1), 1-(3,5-bis(4,4-bis(octyloxy)butoxy)phenyl)-N,N-dimethylmethanamine, 1-(3,5-bis((Z)-octadec-9-en-1-yloxy)phenyl)-N,N-dimethylmethanamine, 1-(3,5-bis((9Z,12Z)-octadeca-9,12-dien-1-yloxy)phenyl)-N,Ndimethylmethanamine, 1-(3-((6,6-bis((2-propylpentyl)oxy)hexanoyl)oxy)-2-(((1,4-dimethylpiperidine-4-carbonyl)oxy)methyl)propyl)8-methyloctanedioate, 1-(3-((6,6-bis((2-propylpentyl)oxy)hexanoyl)oxy)-2-(((1,4-dimethylpiperidine-4-carbonyl)oxy)methyl)propyl)10-octyldecanedioate, 1-(3-((4,4-bis(octyloxy)butanoyl)oxy)-2-(((1-methylpyrrolidine-3-carbonyl)oxy)methyl)propyl)8-methyloctanedioate, 1-(3-((1,3-dimethylpyrrolidine-3-carbonyl)oxy)-2-(((9Z,12Z)-octadeca-9,12-dienoyloxy)methyl)propyl)10-octyldecanedioate, 1-(2,3-Di((8Z,11Z)-heptadeca-8,11-dien-1-yl)cyclopropyl)-N,N-dimethylmethanamine (21-6), 1-(2,2-Di((9Z,12Z)-octadeca-9,12-dien-1-yl)cyclopropyl)-N,N-dimethylmethanamine (21-1), 1-(2-(((3S,10R,13R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yldisulfanyl)ethyl)guanidine (HGT4002), 1-(16-(((4,4-bis(octyloxy)butanoyl)oxy)methyl)-9-dodecyl-2-methyl-7,13-dioxo-6,8,12,14-tetraoxa-2-azaheptadecan-17-yl)8-methyloctanedioate, 1-((6,6-bis(octyloxy)hexanoyl)oxy)pentadecan-3-yl1,4-dimethylpiperidine-4-carboxylate, 1-((6,6-bis(octyloxy)hexanoyl)oxy)pentadecan-3-yl1,3-dimethylpyrrolidine-3-carboxylate, 1-((2R,3S,5R)-3-(bis(hexadecyloxy)methoxy)-5-(5-methyl-2,4-dioxo-3,4-dihydropyrimidin-1(2H)-yl)tetrahydrofumethanesulfonate, 0,0′-ditetradecanoyl-N-(α-trimethylammonioacetyl)diethanolaminechloride (DC-6-14), 0,0′-((5-((dimethylamino)methyl)-1,3-phenylene)bis(methylene))di((9Z,12Z)-octadeca-9,12-dien-1-yl)disuccinate, 0,0′-((5-((dimethylamino)methyl)-1,3-phenylene)bis(methylene))bis(10-(octanoyloxy)decyl)disuccinate, 0′,0-((5-((dimethylamino)methyl)-1,3-phenylene)bis(methylene))8-dinonyldioctanedioate, 0′,0-(((5-((dimethylamino)methyl)-1,3-phenylene)bis(oxy))bis(propane-3,1-diyl))9-dioctyldinonanedioate, [(2-Morpholine-4-yl-ethylcarbamoyl)methyl]-carbamic acid cholesteryl ester (Chol-C3N-Mo2), (Z)-undec-2-en-1-yl6-(3-(decyloxy)-2-(dimethylamino)propoxy)hexanoate, (Z)-undec-2-en-1-yl6-(2-(dimethylamino)-3-(octyloxy)propoxy)hexanoate, (Z)-undec-2-en-1-yl6-(2-(dimethylamino)-3-(nonyloxy)propoxy)hexanoate, (Z)-undec-2-en-1-yl6-(2-(dimethylamino)-3-(hexyloxy)propoxy)hexanoate, (Z)-undec-2-en-1-yl6-(2-(dimethylamino)-3-(heptyloxy)propoxy)hexanoate, (Z)-undec-2-en-1-yl6-(2-(dimethylamino)-3-(4-methoxy-4-oxobutoxy)propoxy)hexanoate, (Z)-undec-2-en-1-yl6-(2-(dimethylamino)-3-((5-methoxy-5-oxopentyl)oxy)propoxy)hexanoate, (Z)-Pent-2-en-1-yl4-((2-(4-(N-(2-(dinonylamino)ethyl)-N-nonylglycyl)piperazin-I-yl)-2-oxoethyl)(nonyl)amino)butanoate, (Z)-Non-2-en-1-yl4-((2-(4-(N-(2-(dinonylamino)ethyl)-N-nonylglycyl)piperazin-I-yl)-2-oxoethyl)(tetradecyl)amino)butanoate, (Z)-N1-(2-(4-(2-(Dodec-6-en-1-yl(dodecyl)amino)ethyl)piperazin--yl)ethyl)-N,N2,N2-tridodecylethane-1,2-diamine, (Z)-methyl8-(2-(dimethylamino)-3-(octadec-9-en-I-yloxy)propoxy)octanoate, (Z)-methyl8-(2-(dimethylamino)-3-((6-oxo-6-(undec-2-en-1-yloxy)hexyl)oxy)propoxy)octanoate, (Z)-methyl7-(2-(dimethylamino)-3-(octadec-9-en-1-yloxy)propoxy)heptanoate, (Z)-methyl7-(2-(dimethylamino)-3-((6-oxo-6-(undec-2-en-1-yloxy)hexyl)oxy)propoxy)heptanoate, (Z)-methyl6-(2-(dimethylamino)-3-(octadec-9-en-1-yloxy)propoxy)hexanoate, (Z)-methyl6-(2-(dimethylamino)-3-((6-oxo-6-(undec-2-en-1-yloxy)hexyl)oxy)propoxy)hexanoate, (Z)-methyl5-(2-(dimethylamino)-3-(octadec-9-en-1-yloxy)propoxy)pentanoate, (Z)-methyl4-(2-(dimethylamino)-3-(octadec-9-en-1-yloxy)propoxy)butanoate, (Z)-methyl17-(2-(dimethylamino)-3-(octyloxy)propoxy)heptadec-8-enoate, (Z)-methyl16-(3-(decyloxy)-2-(dimethylamino)propoxy)hexadec-7-enoate, (Z)-methyl16-(2-(dimethylamino)-3-(nonyloxy)propoxy)hexadec-7-enoate, (Z)-methyl16-(2-(dimethylamino)-3-(hexyloxy)propoxy)hexadec-7-enoate, (Z)-methyl16-(2-(dimethylamino)-3-(heptyloxy)propoxy)hexadec-7-enoate, (Z)-methyl16-(2-(dimethylamino)-3-(4-methoxy-4-oxobutoxy)propoxy)hexadec-7-enoate, (Z)-methyl16-(2-(dimethylamino)-3-((8-methoxy-8-oxooctyl)oxy)propoxy)hexadec-7-enoate, (Z)-methyl16-(2-(dimethylamino)-3-((7-methoxy-7-oxoheptyl)oxy)propoxy)hexadec-7-enoate, (Z)-methyl16-(2-(dimethylamino)-3-((6-methoxy-6-oxohexyl)oxy)propoxy)hexadec-7-enoate, (Z)-methyl16-(2-(dimethylamino)-3-((5-methoxy-5-oxopentyl)oxy)propoxy)hexadec-7-enoate, (Z)-4-((2-(4-(N-(2-(dinonylamino)ethyl)-N-nonylglycyl)piperazin-I-yl)-2-oxoethyl)(tetradecyl)amino)but-2-en-1-y1nonanoate(Z)-4-Hydroxybut-2-en-1-ylnonanoate, (Z)-3-((2-(4-(N-(2-(Dinonylamino)ethyl)-N-nonylglycyl)piperazin-1-yl)-2-oxoethyl)(tetradec-9-en-1-yl)amino)propyldecanoate(Z)-Tetradec-9-en-1-ylmethanesulfonate, (Z)-2-((4-(((3-(dimethylamino)propoxy)carbonyl)oxy)hexadecanoyl)oxy)propane-1,3-diyldioleate, (Z)-2-((2-(Didodecyl amino)ethyl) (dodec-6-en-1-yl)amino)ethan-1-ol (20-23), (Z)-((5-((dimethylamino)methyl)-1,3-phenylene)bis(oxy))bis(butane-4,1-diyl)dioleate, (S)-4-(3-((S)-3,4-bis(octanoyloxy)butoxy)-5-((dimethylamino)methyl)phenoxy)butane-1,2-diyldioctanoate, (S)-2-Amino-3-hydroxy-N,N-bis(2-(((Z)-octadeca-9-en-1-yl)oxy)ethyl)propanamide (CL-21), (R)-4-(3-((S)-3,4-bis(octanoyloxy)butoxy)-5-((dimethylamino)methyl)phenoxy)butane-1,2-diyldioctanoate, (R)-4-(3-((R)-3,4-bis(octanoyloxy)butoxy)-5-((dimethylamino)methyl)phenoxy)butane-1,2-diyldioctanoate, (1-(N-(2-(Dinonylamino)ethyl)-N-nonylglycyl)pyrrolidin-3-yl)methyldinonylglycinate, (1-(2,3-linoleyloxypropoxy)-2-(Iinoleyloxy)-(7V,N-dimethyl)-propyl-3-amine) (TLinDMA), (9Z.9′Z.12Z.12′Z)-((5-((dimethylamino)methyn-2-methyl-1.3-phenylene)bis(oxy))bis(butane-4,1-diyl)bis(octadeca-9,12-dienoate), (9Z.9′Z.12Z.12′Z)-((5-((dimethylamino)methyn-1.3-phenylene)bis(oxy))bis(octane-8,1-diyl)bis(octadeca-9,12-dienoate), (9Z.9′Z.12Z.12′Z)-((5-((diethylamino)methyn-2-methyl-1.3-phenylene)bis(oxy))bis(butane-4,1-diyl)bis(octadeca-9,12-dienoate), (9Z.12Z)-4-(3-((dimethylamino)methyn-5-(4-((3-octylundecanoyl)oxy)butoxy)phenoxy)butyloctadeca-9,12-dienoate, (9Z,9′Z,12Z,12′Z,15Z,15′Z)-((5-((dimethylamino)methyl)-1,3-phenylene)bis(oxy))bis(butane-4,1-diyl)bis(octadeca-9,12,15-trienoate), (9Z,9′Z,12Z,12′Z)-5-(((3-(dimethylamino)propanoyl)oxy)methyl)-1,3-phenylenebis(octadeca-9,12-dienoate), (9Z,9′Z,12Z,12′Z)-2-(9-dodecyl-2-methyl-7,12-dioxo-6,8,13-trioxa-2-azatetradecan-14-yl)propane-1,3-diylbis(octadeca-9,12-dienoate), (9Z,9′Z,12Z,12′Z)-2-((4-(((3-(dimethylamino)propoxy)carbonyl)oxy)hexadecanoyl)oxy)propane-1,3-diylbis(octadeca-9,12-dienoate) (Cationic Lipid A2), (9Z,9′Z,12Z,12′Z)-2-(((4-(pyrrolidin-1-yl)butanoyl)oxy)methyl)propane-1,3-diylbis(octadeca-9,12-dienoate), (9Z,9′Z,12Z,12′Z)-2-(((4-(piperidin-1-yl)butanoyl)oxy)methyl)propane-1,3-diylbis(octadeca-9,12-dienoate), (9Z,9′Z,12Z,12′Z)-2-(((4-(dimethylamino)butanoyl)oxy)methyl)propane-1,3-diylbis(octadeca-9,12-dienoate), (9Z,9′Z,12Z,12′Z)-2-(((3-morpholinopropanoyl)oxy)methyl)propane-1,3-diylbis(octadeca-9,12-dienoate), (9Z,9′Z,12Z,12′Z)-2-(((3-(diethylamino)propanoyl)oxy)methyl)propane-1,3-diylbis(octadeca-9,12-dienoate), (9Z,9′Z,12Z,12′Z)-2-(((3-(4-methylpiperazin-1-yl)propanoyl)oxy)methyl)propane-1,3-diylbis(octadeca-9,12-dienoate), (9Z,9′Z,12Z,12′Z)-2-(((1,3-dimethylpyrrolidine-3-carbonyl)oxy)methyl)propane-1,3-diylbis(octadeca-9,12-dienoate), (9Z,9′Z,12Z,12′Z)-2-(((1-(cyclopropylmethyl)piperidine-4-carbonyl)oxy)methyl)propane-1,3-diylbis(octadeca-9,12-dienoate), (9Z,9′Z,12Z,12′Z)-2-((((3-(diethylamino)propoxy)carbonyl)oxy)methyl)-2-(((9Z,12Z)-octadeca-9,12-dienoyloxy)methyl)propane-1,3-diylbis(octadeca-9,12-dienoate), (9Z,9′Z,12Z,12′Z)-(5-((dimethylamino)methyl)-1,3-phenylene)bis(methylene)bis(octadeca-9,12-dienoate), (9Z,9′Z,12Z,12′Z)-(5-((((3-(diethylamino)propoxy)carbonyl)oxy)methyl)-1,3-phenylene)bis(methylene)bis(octadeca-9,12-dienoate), (9Z,9′Z,12Z,12′Z)-((5-(pyrrolidin-1-ylmethyl)-1,3-phenylene)bis(oxy))bis(butan4,1-diyl)bis(octadeca-9,12-dienoate), (9Z,9′Z,12Z,12′Z)-((5-(3-morpholinopropyl)-1,3-phenylene)bis(oxy))bis(butane4,1-diyl)bis(octadeca-9,12-dienoate), (9Z,9′Z,12Z,12′Z)-((5-(3-(piperidin-1-yl)propyl)-1,3-phenylene)bis(oxy))bis(butane-4,1-diyl)bis(octadeca-9,12-dienoate), (9Z,9′Z,12Z,12′Z)-((5-(3-(dimethylamino)propyl)-1,3-phenylene)bis(oxy))bis(butane-4,1-diyl)bis(octadeca-9,12-dienoate), (9Z,9′Z,12Z,12′Z)-((5-((dimethylamino)methyl)-1,3-phenylene)bis(oxy))bis(propane-3,1-diyl)bis(octadeca-9,12-dienoate), (9Z,9′Z,12Z,12′Z)-((5-((dimethylamino)methyl)-1,3-phenylene)bis(oxy))bis(butane-4,1-diyl)bis(octadeca-9,12-dienoate), (9Z,9′Z,12Z,12′Z)-((5-((dimethylamino)methyl)-1,3-phenylene)bis(oxy))bis(hexane-6,1-diyl)bis(octadeca-9,12-dienoate), (9Z,9′Z,12Z,12′Z)-((5-((dimethylamino)methyl)-1,3-phenylene)bis(oxy))bis(ethane-2,1-diyl)bis(octadeca-9,12-dienoate), (9Z,9′Z,12Z,12′Z)-((5-((3-hydroxyazetidin-1-yl)methyl)-1,3-phenylene)bis(oxy))bis(butane-4,1-diyl)bis(octadeca-9,12-dienoate), (9Z,9′Z,12Z,12′Z)-(((5-((dimethylamino)methyl)-1,3-phenylene)bis(methylene))bis(oxy))bis(4-oxobutane-4,1-diyl)bis(octadeca-9,12-dienoate), (9Z,9′Z,12Z,12′Z)-((((5-((diethylamino)methyl)-1,3-phenylene)bis(oxy))bis(ethane-2,1-diyl))bis(oxy))bis(ethane-2,1-diyl)bis(octadeca-9,12-dienoate), (9Z,9,Z,12Z,12,Z,15Z,15,Z)-2-((4-(((3-(dimethylamino)propoxy)carbonyl)oxy)hexadecanoyl)oxy)propane-1,3-diylbis(octadeca-9,12,15-trienoate), (9Z,9,Z,12Z,12,Z)-2-((4-(((3-(dimethylamino)propoxy)carbonyl)oxy)hexadecanoyl)oxy)propane-1,3-diylbis(octadeca-9,12-dienoate), (9Z,9,Z,12Z,12,Z)-2-((4-(((3-(diethylamino)propoxy)carbonyl)oxy)hexadecanoyl)oxy)propane-1,3-diylbis(octadeca-9,12-dienoate), (9Z,9,Z,12Z,12,Z)-2-((2-(((3-(dimethylamino)propoxy)carbonyl)oxy)tetradecanoyl)oxy)propane-1,3-diylbis(octadeca-9,12-dienoate), (9Z,12Z)-N-Methyl-N-(2-(((9Z,12Z)-octadeca-9,12-dien-1-yl)oxy)ethyl)octadeca-9,12-dien-1-amine (CL-17), (9Z,12Z)-N-(3-(((9Z,12Z)-octadeca-9,12-dien-1-yl)oxy)propyl)octadeca-9,12-dien-1-amine (CL-18), (9Z,12Z)-N-(2-Methoxyethyl)-N-((9Z,12Z)-octadeca-9,12-dien-1-yl)octadeca-9,12-dien-1-amine (17-9), (9Z,12Z)-N-(2-(4-Dodecylpiperazin-1-yl)ethyl)-N-((9Z,12Z)-octadeca-9,12-dien-1-yl)octadeca-9,12-dien-1-amine (19-6), (9Z,12Z)-N-(2-(((Z)-Octadeca-9-en-1-yl)oxy)ethyl)octadeca-9,12-dien-1-amine (CL-7), (9Z,12Z)-N-((9Z,12Z)-Octadeca-9,12-dien-1-yl)-N-(2-(piperazin-1-yl)ethyl)octadeca-9,12-dien-1-amine (19-3), (9Z,12Z)-di((9Z,12Z)-octadeca-9,12-dien-1-yl)amine (CL-15), (9Z,12Z)-5-(((3-(dimethylamino)propoxy)carbonyl)oxy)-7-octylpentadecyloctadeca-9,12-dienoate, (9Z,12Z)-4-(3-((dimethylamino)methyl)-5-(4-(oleoyloxy)butoxy)phenoxy)butyloctadeca-9,12-dienoate, (9Z,12Z)-3-(3-((dimethylamino)methyl)-5-(3-((3-octylundecanoyl)oxy)propoxy)phenoxy)propyloctadeca-9,12-dienoate, (9Z,12Z)-3-(2,2-bis(heptyloxy)acetoxy)-2-((((2-(dimethylamino)ethoxy)carbonyl)oxy)methyl)propyloctadeca-9,12-dienoate, (9Z,12Z)-3-((6,6-bis(octyloxy)hexanoyl)oxy)-2-((((3-(diethylamino)propoxy)carbonyl)oxy)methyl)propyloctadeca-9,12-dienoate, (9Z,12Z)-3-((4,4-bis(octyloxy)butanoyl)oxy)-2-(((3-(dimethylamino)propanoyl)oxy)methyl)propyloctadeca-9,12-dienoate, (9Z,12Z)-3-((4,4-bis(octyloxy)butanoyl)oxy)-2-((((3-(pyrrolidin-1-yl)propoxy)carbonyl)oxy)methyl)propyloctadeca-9,12-dienoate, (9Z,12Z)-3-((4,4-bis(octyloxy)butanoyl)oxy)-2-((((3-(dimethylamino)propyl)carbamoyl)oxy)methyl)propyloctadeca-9,12-dienoate, (9Z,12Z)-3-((4,4-bis(octyloxy)butanoyl)oxy)-2-((((3-(diethylamino)propoxy)carbonyl)oxy)methyl)propyloctadeca-9,12-dienoate (Lipid A1), (9Z,12Z)-3-((4,4-bis(octyloxy)butanoyl)oxy)-2-((((3-(4-methylpiperazin-1-yl)propoxy)carbonyl)oxy)methyl)propyloctadeca-9,12-dienoate, (9Z,12Z)-3-((4,4-bis(octyloxy)butanoyl)oxy)-2-(((((1-methylpyrrolidin-3-yl)oxy)carbonyl)oxy)methyl)propyloctadeca-9,12-dienoate, (9Z,12Z)-3-((4,4-bis(octyloxy)butanoyl)oxy)-2-(((((1-methylpiperidin-4-yl)oxy)carbonyl)oxy)methyl)propyloctadeca-9,12-dienoate, (9Z,12Z)-3-((4,4-bis(octyloxy)butanoyl)oxy)-2-(((((1-methylpiperidin-4-yl)methoxy)carbonyl)oxy)methyl)propyloctadeca-9,12-dienoate, (9Z,12Z)-3-((4,4-bis(octyloxy)butanoyl)oxy)-2-(((((1-methylazetidin-3-yl)oxy)carbonyl)oxy)methyl)propyloctadeca-9,12-dienoate, (9Z,12Z)-3-((4,4-bis(octyloxy)butanoyl)oxy)-2-(((((1-ethylpiperidin-4-yl)oxy)carbonyl)oxy)methyl)propyloctadeca-9,12-dienoate, (9Z,12Z)-3-((4,4-bis(octyloxy)butanoyl)oxy)-2-(((((1-ethylpiperidin-3-yl)methoxy)carbonyl)oxy)methyl)propyloctadeca-9,12-dienoate, (9Z,12Z)-3-((4,4-bis(octyloxy)butanoyl)oxy)-2-(((((1,2,2,6,6-pentamethylpiperidin-4-yl)oxy)carbonyl)oxy)methyl)propyloctadeca-9,12-dienoate, (9Z,12Z)-3-(((3-dimethylamino)propoxy)carbonyl)oxy)-13-(octanoyloxy)tridecyloctadeca-9,12-dienoate, (9Z,12Z)-3-(((3-(ethyl(methyl)amino)propoxy)carbonyl)oxy)pentadecyloctadeca-9,12-dienoate, (9Z,12Z)-3-(((3-(dimethylamino)propoxy)carbonyl)oxy)pentadecyloctadeca-9,12-dienoate, (9Z,12Z)-3-(((3-(diethylamino)propoxy)carbonyl)oxy)pentadecyloctadeca-9,12-dienoate, (9Z,12Z)-3-(((3-(diethylamino)propoxy)carbonyl)oxy)-2-(((9-pentyltetradecanoyl)oxy)methyl)propyloctadeca-9,12-dienoate, (9Z,12Z)-3-(((3-(diethylamino)propoxy)carbonyl)oxy)-2-(((7-hexyltridecanoyl)oxy)methyl)propyloctadeca-9,12-dienoate, (9Z,12Z)-3-(((3-(diethylamino)propoxy)carbonyl)oxy)-2-(((5-heptyldodecanoyl)oxy)methyl)propyloctadeca-9,12-dienoate, (9Z,12Z)-3-(((3-(diethylamino)propoxy)carbonyl)oxy)-2-(((3-octylundecanoyl)oxy)methyl)propyloctadeca-9,12-dienoate, (9Z,12Z)-3-(((3-(diethylamino)propoxy)carbonyl)oxy)-2-(((2-heptylundecanoyl)oxy)methyl)propyloctadeca-9,12-dienoate, (9Z,12Z)-3-(((2-(dimethylamino)ethoxy)carbonyl)oxy)pentadecyloctadeca-9,12-dienoate, (9Z,12Z)-2-(((11Z,14Z)-2-((3-(dimethylamino)propanoyl)oxy)icosa-11,14-dien-1-yl)oxy)ethyloctadeca-9,12-dienoate, (9Z,12Z)-10-dodecyl-3-ethyl-14-(2-((9Z,12Z)-octadeca-9,12-dienoyloxy)ethyl)-8,13-dioxo-7,9-dioxa-3,14-diazahexadecan-16-yloctadeca-9,12-dienoate, (9Z,12Z)-(12Z,15Z)-3-((3-(dimethylamino)propanoyl)oxy)henicosa-12,15-dien-1-yloctadeca-9,12-dienoate, (8Z,8′Z)-((5-((dimethylamino)methyl)-1,3-phenylene)bis(oxy))bis(hexane-bis(dodec-8-enoate), (6Z,9Z,28Z,31Z)-heptatriaconta-6,9,28,31-tetraen-19-yl4-(dimethylamino)butanoate), (6Z,9Z,28Z,31Z)-Heptatriaconta-6,9,28,31-tetraen-19-yl4-(dimethylamino)butanoate (CL-5), (6Z,9Z,28Z,31Z)-heptatriacont-6,9,28,31-tetraene-19-yl4-(dimethylamino)butanoate (DLin-MC3-DMA), (6Z,16Z)-12-((Z)-dec-4-enyl)docosa-6,16-dien-11-yl5-(dimethylamino)pentanoate, (6Z,16Z)-12-((Z)-dec-4-enyl)docosa-6,16-dien-11-yl5-(dimethylamino)pentanoat, (6Z,16Z)-12-((Z)-dec-4-enyl)docosa-6,16-dien-11-yl5-(dimethylamino)pentanoate, (6Z,16Z)-12-((Z)-dec-4-en-1-yl)docosa-6,16-dien-11-yl5-(dimethylamino)pentanoate (Lipid 1), (5-((dimethylamino)methyl)-1,3-phenylene)bis(methylene)ditridecanoate, (5-((dimethylamino)methyl)-1,3-phenylene)bis(methylene)bis(9-pentyltetradecanoate), (5-((dimethylamino)methyl)-1,3-phenylene)bis(methylene)bis(8-(octanoyloxy)octanoate), (5-((dimethylamino)methyl)-1,3-phenylene)bis(methylene)bis(7-hexyltridecanoate), (5-((dimethylamino)methyl)-1,3-phenylene)bis(methylene)bis(6-(((nonyloxy)carbonyl)oxy)hexanoate), (5-((dimethylamino)methyl)-1,3-phenylene)bis(methylene)bis(5-heptyldodecanoate), (5-((dimethylamino)methyl)-1,3-phenylene)bis(methylene)bis(10-(octanoyloxy)decanoate), (4-((6Z,9Z,28Z,31Z)-heptatriaconta-6,9,28,31-tetraen-19-yloxy)-N,N-dimethylbutan-1-amine (MC4 Ether), (3R,4R)-3,4-bis((Z)-hexadec-9-enyloxy)-1-methylpyrrolidine, (3R,4R)-3,4-bis(((Z)-hexadec-9-en-1-yl)oxy)-1-methylpyrrolidine(CompoundCL-12) (CL-12), (3aR5s,6aS)-N,N-dimethyl-2,2-di((9Z,12Z)-octadeca-9,12-dienyl)tetrahydro-3aH-cyclopentad1,3dioxol-5-amine (ALN100), (3aR,5s,6aS)-N,N-dimethyl-2,2-di((9Z,12Z)-octadeca-9,12-dienyl)tetrahydro-3aHcyclopenta[d][1,3]dioxol-5-amine (ALN1001), (3-((6Z,9Z,28Z,31Z)-heptatriaconta-6,9,28,31-tetraen-19-yloxy)-N,N-dimethylpropan-1-amine) (1-BI 1), (3-((6Z,9Z,28Z,31Z)-heptatriaconta-6,9,28,31-tetraen-19-yloxy)-N,N-dimethylpropan-1-amine (MC3 Ether), (2Z)-undec-2-en-1-yl8-{[4-(dimethylamino)butanoyl]oxy}heptadecanoate, (2Z)-non-2-en-1-yl10-{[4-(dimethylamino)butanoyl]oxy}nonadecanoate, (2Z)-hept-2-en-1-yl12-{[4-(dimethylamino)butanoyl]oxy}henicosanoate, (2SN,N-dimethyl-1-[(9Z,12Z)-octadeca-9,12-dien-1-yloxy]decan-2-amine(Compound11), (2S,12Z,15Z)-N,N-dimethyl-1-(octyloxy)henicosa-12,15-dien-2-amine, (2S)-N.N-dimethyl-1-[(9Z,12Z)-octadeca-9,12-dien-1-yloxy]nonan-2-amine, (2S)-N,N-dimethyl-1-[(9Z,12Z)-octadeca-9,12-dien-1-yloxy]undecan-2-amine, (2S)-N,N-dimethyl-1-[(9Z,12Z)-octadeca-9,12-dien-1-yloxy]tridecan-2-amine, (2S)-N,N-dimethyl-1-[(9Z,12Z)-octadeca-9,12-dien-1-yloxy]dodecan-2-amine, (2S)-N,N-dimethyl-1-({8-[(IR,2R)-2-{[(IS,2S)-2-pentylcyclopropyl]methyl}cyclopropyl]octyl}oxy)tridecan-2-amine, (2S)-3-((4,4-bis(octyloxy)butanoyl)oxy)-2-(((9Z,12Z)-octadeca-9,12-dienoyloxy)methyl)propyl1-methylpyrrolidine-2-carboxylate, (2S)-1-[(9Z,12Z)-octadeca-9,12-dien-1-yloxy]undecan-2-amine, (2S)-1-[(9Z,12Z)-octadeca-9,12-dien-1-yloxy]nonan-2-amine, (2S)-1-[(9Z,12Z)-octadeca-9,12-dien-1-yloxy]dodecan-2-amine, (2S)-1-[(9Z,12Z)-octadeca-9,12-dien-1-yloxy]decan-2-amine, (2S)-1-({6-[3B))-cholest-5-en-3-yloxy]hexyl}oxy)-N,N-dimethyl-3-[(9Z)-octadec-9-en-1-yloxy]propan-2-amine, (2S)-1-((6,6-bis(octyloxy)hexanoyl)oxy)pentadecan-3-yl1-methylpyrrolidine-2-carboxylate, (2R,12Z,15Z)-N,N-dimethyl-1-(undecyloxy)henicosa-12,15-dien-2-amine, (2R,12Z,15Z)-1-(hexyloxy)-N,N-dimethylhenicosa-12,15-dien-2-amine, (2R,12Z,15Z)-1-(hexadecyloxy)-N,N-dimethylhenicosa-12,15-dien-2-amine, (2R,12Z,15Z)-1-(decyloxy)-N,N-dimethylhenicosa-12,15-dien-2-amine, (2R)-N,N-dimethyl-1-[(9Z,12Z)-octadeca-9,12-dien-1-yloxy]dodecan-2-amine, (2R)-3-((4,4-bis(octyloxy)butanoyl)oxy)-2-(((9Z,12Z)-octadeca-9,12-dienoyloxy)methyl)propyl1-methylpyrrolidine-2-carboxylate, (2R)-2-({8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), (2R)-1-[(9Z,12Z)-octadeca-9,12-dien-1-yloxy]dodecan-2-amine, (2R)-1-((6,6-bis(octyloxy)hexanoyl)oxy)pentadecan-3-ylpyrrolidine-2-carboxylate, (2R)-1-((6,6-bis(octyloxy)hexanoyl)oxy)pentadecan-3-yl1-methylpyrrolidine-2-carboxylate, (2-octylcyclopropyl)methyl8-{[4-(dimethylamino)butanoyl]oxy}heptadecanoate, (2-octylcyclopropyl)methyl6-(3-(decyloxy)-2-(dimethylamino)propoxy)hexanoate, (2-octylcyclopropyl)methyl6-(2-(dimethylamino)-3-(octyloxy)propoxy)hexanoate, (2-octylcyclopropyl)methyl6-(2-(dimethylamino)-3-(nonyloxy)propoxy)hexanoate, (2-octylcyclopropyl)methyl6-(2-(dimethylamino)-3-(hexyloxy)propoxy)hexanoate, (2-octylcyclopropyl)methyl6-(2-(dimethylamino)-3-(heptyloxy)propoxy)hexanoate, (2-octylcyclopropyl)methyl6-(2-(dimethylamino)-3-(4-methoxy-4-oxobutoxy)propoxy)hexanoate, (2-octylcyclopropyl)methyl6-(2-(dimethylamino)-3-((5-methoxy-5-oxopentyl)oxy)propoxy)hexanoate, (2JR)-2-{4-[(3p)-cholest-5-en-3-yloxy]butoxy}-Λr\hat{ }dimethyl-3-[(9Z,12Z)-octadeca-9-12-dien-1-yloxyjpropan-1-amine (Butyl-CLinDMA (2R)), (2-hexylcyclopropyl)methyl10-{[4-(dimethylamino)butanoyl]oxy}nonadecanoate, (2-butylcyclopropyl)methyl12-{[4-(dimethylamino)butanoyl]oxy}henicosanoate, (2-amino-N,N-dihexadecyl-3-(1H-imidazol-5-yl)propenamide, (25)-2-{4-[(3p)-cholest-5-en-3-yloxy]butoxy}-iVy/V-dimethyl-3-[(9Z,12Z)-octadeca-9,12-dien-1-yloxy]propan-1-amine (Butyl-CLinDMA (2S)), (24Z)-N,N-dimethyltritriacont-24-en-10-amine, (22Z,25Z)-N,N-dimethylhentriaconta-22,25-dien-10-amine, (22Z)-N,N-dimethylhentriacont-22-en-10-amine, (21Z,24Z)-N,N-dimethyltriaconta-21,24-dien-9-amine, (20Z,23Z)-nonacosa-20,23-dien-10-yl4-(dimethylamino)butanoate, (20Z,23Z)-N-ethyl-N-methylnonacosa-20,23-dien-10-amine, (20Z,23Z)-N,N-dimethylnonacosa-20,23-dien-10-amine, (20Z)-N,N-dimethylnonacos-20-en-10-amine, (20Z)-N,N-dimethylheptacos-20-en-10-amine, (2,6-bis((9Z,12Z)-octadeca-9,12-dien-1-yloxy)pyridin-4-yl)methyl3-(dimethylamino)propanoate, (1-Methylpiperidin-3-yl)methyldi((11Z,14Z)-icosa-11,14-dien-1-yl)carbamate (CL-19), (19Z,22Z)-N,N-dimethyloctacosa-19,22-dien-7-amine, (19Z,22Z)-N,N-dimeihyloctacosa-19,22-dien-9-amine, (18Z,21Z)-N,N-dimethylheptacosa-18,21-dien-8-amine, (18Z,21Z)-N,N-dimethylheptacosa-18,21-dien-10-amine, (18Z)-N,N-dimethylheptacos-18-en-10-amine, (18Z)-heptacos-18-en-10-yl4-(dimethylamino)butanoate, (17Z,20Z)-N,N-dimethylhexacosa-17,20-dien-7-amine, (17Z,20Z)-N,N-dimemylhexacosa-17,20-dien-9-amine, (17Z)-N,N-dimethylnonacos-17-en-10-amine, (17Z)-N,N-dimethylhexacos-17-en-9-amine, (16Z,19Z)-N,N-dimethylpentacosa-16,19-dien-6-amine, (16Z,19Z)-N,N-dimethylpentacosa-16,19-dien-8-amine, (16Z)-N,N-dimethylpentacos-16-en-8-amine, (15Z,18Z)-N,N-dimethyltetracoda-15,18-dien-5-amine, (15Z,18Z)-N,N-dimethyltetracosa-15,18-dien-7-amine, (15Z,18Z)-N,N-dimethyl-6-(9Z,12Z)-octadeca-9,12-dien-1-yl)tetracosa-15,18-dien-1-amine (HGT5000), (15Z,18Z)-N,N-dimethyl-6-((9Z,12Z)-octadeca-9,12-dien-1-yl)tetracosa-4,15,18-trien-1-amine (HGT5001), (15Z)-N,N-dimethylheptacos-15-en-10-amine, (14Z,17Z)-N,N-dimethyltricosa-14,17-dien-6-amine, (14Z,17Z)-N,N-dimethyltricosa-14,17-dien-4-amine, (14Z)-N,N-dimethylnonacos-14-en-10-amine, (13Z,16Z)-N,N-dimethyldocosa-13,16-dien-5-amine, (13Z,16Z)-N,N-Dimethyl-4-((9Z,12Z)-octadeca-9,12-dien-1-yl)docosa-3,13,16-trien-1-amine (CL-20), (13Z,16Z)-N,N-dimethyl-3-nonyldocosa-13,16-dien-1-amine, (13Z,16Z)-4-(((3-(dimethylamino)propoxy)carbonyl)oxy)docosa-13,16-dien-1-ylheptadecan-9-ylsuccinate, (13Z,16Z)-4-(((3-(diethylamino)propoxy)carbonyl)oxy)docosa-13,16-dien-1-yl2,2-bis(heptyloxy)acetate, (13Z,16Z)-4-(((2-(dimethylamino)ethoxy)carbonyl)oxy)docosa-13,16-dien-1-yl2,2-bis(heptyloxy)acetate, (12Z,15Z)-N,N-dimethylhenicosa-12,15-dien-4-amine, (12Z,15Z)-N,N-dimethyl-2-nonylhenicosa-12,15-dien-1-amine, (12Z,15Z)-3-((4-(dimethylamino)butanoyl)oxy)henicosa-12,15-dien-1-yl9-pentyltetradecanoate, (12Z,15Z)-3-((4-(dimethylamino)butanoyl)oxy)henicosa-12,15-dien-1-yl7-hexyltridecanoate, (12Z,15Z)-3-((4-(dimethylamino)butanoyl)oxy)henicosa-12,15-dien-1-yl5-heptyldodecanoate, (12Z,15Z)-3-((4-(dimethylamino)butanoyl)oxy)henicosa-12,15-dien-1-yl3-octylundecanoate,formatesalt, (12Z,15Z)-3-(((3-(diethylamino)propoxy)carbonyl)oxy)henicosa-12,15-dien-1-yl6,6-bis(octyloxy)hexanoate, (12Z,15Z)-1-((((9Z,12Z)-octadeca-9,12-dien-1-yloxy)carbonyl)oxy)henicosa-12,15-dien-3-yl3-(dimethylamino)propanoate, (11Z,14Z)-2-(Dimethylamino)-2-((9Z,12Z)-octadeca-9,12-dien-1-yl)icosa-11,14-dien-1-ol (CL-64), (11Z,14Z)-2-((Dimethylamino)methyl)-2-((9Z,12Z)-octadeca-9,12-dien-1-yl)icosa-11,14-dien-1-ol (CL-63), (11E,20Z,23Z)-N,N-dimethylnonacosa-11,20,23-trien-10-amine, ((5-((dimethylamino)methyl)-1,3-phenylene)bis(oxy))bis(octane-8,1-diyl)bis(decanoate) (Lipid B), ((5-((dimethylamino)methyl)benzene-1,2,3-triyl)tris(oxy))tris(decane10,1-diyl)trioctanoate, ((5-((dimethylamino)methyl)-1,3-phenylene)bis(oxy))bis(propane-3,1-diyl)bis(3-octylundecanoate), ((5-((dimethylamino)methyl)-1,3-phenylene)bis(oxy))bis(octane-8,1-diyl)dioctanoate, ((5-((dimethylamino)methyl)-1,3-phenylene)bis(oxy))bis(octane-8,1-diyl)dihexanoate, ((5-((dimethylamino)methyl)-1,3-phenylene)bis(oxy))bis(octane-8,1-diyl)didodecanoate, ((5-((dimethylamino)methyl)-1,3-phenylene)bis(oxy))bis(octane-8,1-diyl)bis(decanoate), ((5-((dimethylamino)methyl)-1,3-phenylene)bis(oxy))bis(hexane-6,1-diyl)dioctanoate, ((5-((dimethylamino)methyl)-1,3-phenylene)bis(oxy))bis(hexane-6,1-diyl)didodecanoate, ((5-((dimethylamino)methyl)-1,3-phenylene)bis(oxy))bis(hexane-6,1-diyl)bis(decanoate), ((5-((dimethylamino)methyl)-1,3-phenylene)bis(oxy))bis(decane-10,1-diyl)dioctanoate, ((5-((dimethylamino)methyl)-1,3-phenylene)bis(oxy))bis(decane-10,1-diyl)dihexanoate, ((5-((dimethylamino)methyl)-1,3-phenylene)bis(oxy))bis(butane-4,1-diyl)ditetradecanoate, ((5-((dimethylamino)methyl)-1,3-phenylene)bis(oxy))bis(butane-4,1-diyl)bis(9-pentyltetradecanoate), ((5-((dimethylamino)methyl)-1,3-phenylene)bis(oxy))bis(butane-4,1-diyl)bis(7-hexyltridecanoate), ((5-((dimethylamino)methyl)-1,3-phenylene)bis(oxy))bis(butane-4,1-diyl)bis(5-heptyldodecanoate), ((5-((dimethylamino)methyl)-1,3-phenylene)bis(oxy))bis(butane-4,1-diyl)bis(3-octylundecanoate), ((5-((diethylamino)methyl)benzene-1,2,3-triyl)tris(oxy))tris(decane-10,1-diyl)trioctanoate, ((5-(((4-(dimethylamino)butanoyl)oxy)methyl)-1,3-phenylene)bis(oxy))bis(octane-8,1-diyl)bis(decanoate), ((5-(((3-(dimethylamino)propanoyl)oxy)methyl)-1,3-phenylene)bis(oxy))bis(octane-8,1-diyl)bis(decanoate), ((3aR,5s,6aS)-N,N-dimethyl-2,2-di((9Z,12Z)-octadeca-9,12-dienyl)tetrahydro-3aH-cyclopenta[d][1,3]dioxol-5-amine)) (ALNY-100), ((2-((3,S′,4R)-3,4-dihydroxypyrrolidin-1-yl)acetyl)azanediyl)bis(ethane-2,1-diyl)(9Z,9′Z,12Z,12′Z)-bis(octadeca-9,12-dienoate), ((2-((3,S′,4R)-3,4-dihydroxypyrrolidin-1-yl)acetyl)azanediyl)bis(ethane-2,1-diyl)(9Z,9′Z,12Z,12′Z)-bis(octadeca-9,12-dienoate), ((2-(((4-(dimethylamino)butanoyl)oxy)methyl)-1,4-phenylene)bis(oxy))bis(octane-8,1-diyl)bis(decanoate), ((2-(((1-methylpiperidine-4-carbonyl)oxy)methyl)-1,4-phenylene)bis(oxy))bis(octane-8,1-diyl)bis(decanoate), ((2-(((1-isopropylpiperidine-4-carbonyl)oxy)methyl)-1,4-phenylene)bis(oxy))bis(octane-8,1-diyl)bis(decanoate), ((2-((((3-(dimethylamino)propoxy)carbonyl)oxy)methyl)-1,4-phenylene)bis(oxy))bis(octane-8,1-diyl)bis(decanoate), ((2-((((3-(diethylamino)propoxy)carbonyl)oxy)methyl)-1,4-phenylene)bis(oxy))bis(octane-8,1-diyl)bis(decanoate), (((5-((dimethylamino)methyl)-1,3-phenylene)bis(oxy))bis(methylene))bis(propane-3,2,1-triyl)tetraoctanoate, (((5-((dimethylamino)methyl)-1,3-phenylene)bis(oxy))bis(butane-4,1-diyl))bis(propane-3,2,1-triyl)tetraoctanoate, (((5-((dimethylamino)methyl)-1,3-phenylene)bis(methylene))bis(oxy))bis(8-oxooctane-8,1-diyl)bis(decanoate), (((5-((dimethylamino)methyl)-1,3-phenylene)bis(methylene))bis(oxy))bis(6-oxohexane-6,1-diyl)dioctanoate, (((5-((dimethylamino)methyl)-1,3-phenylene)bis(methylene))bis(oxy))bis(6-oxohexane-6,1-diyl)bis(decanoate), ((((5-((dimethylamino)methyl)-1,3-phenylene)bis(oxy))bis(propane-3,1-diyl))bis(oxy))bis(4-oxobutane-4,1-diyl)bis(decanoate), ((((5-((dimethylamino)methyl)-1,3-phenylene)bis(oxy))bis(butane-4,1-diyl))bis(oxy))bis(propane-3,2,1-triyl)tetraoctanoate, N1-[2-((IS)-1-[(3-aminopropyl)amino]-4-[di(3-amino-propyl)amino]butylcarboxamido)ethyl]-3,4-di[oleyloxy]-benzamide (MVL5), histidinyl cholesterol hemisuccinate (Hist-Chol), a-D-Tocopherol hemisuccinoyl, and/or [(2-Morpholine-4-yl-ethylcarbamoyl)-ethyl]-carbamicacidcholesterylesterChol-DMC3N-Mo2[1-Methyl-2-(2-morpholine-4-yl-ethylcarbamoyl)-propyl]-carbamicacidcholesterylester (Chol-C4N-Mo2).

### Non-Cationic Lipid

In some embodiments, the non-cationic lipid may be a fusogenic lipid, an anionic lipid, or a neutral lipid.

In some embodiments, the lipoprotein systems of the present disclosure may include an anionic lipid. An anionic lipid can contain any of a wide range of fatty acid chains in the hydrophobic region. The specific fatty acids incorporated are responsible for the fluidic characteristics of the lipid structure in terms of phase behavior and elasticity. In some cases, divalent cations can be incorporated into an anionic lipid structure. Several divalent cations can be used in anionic lipoplexes such as Ca²⁺, Mg²⁺, Mn²⁺, and Ba²⁺. In some cases, monovalent cations can be used in anionic lipoplexes such as Na⁺ or K⁺. In some embodiments, the anionic lipid may include phosphatidylglycerol, cardiolipin, dialkyl phosphatidylserine, dialkyl phosphatidic acid, N-dodecanoyl phosphatidylethanolamine, N-succinyl phosphatidylethanolamine, N-glutarylphosphatidylethanolamine, lysylphosphatidylglycerol, palmitoyloleyolphosphatidylglycerol (POPG), glycerophosphoinositol monophosphate, glycerophosphoinositol bisphosphate, glycerophosphoinositol trisphosphate, glycerophosphate, a glyceropyrophosphate, glycerophosphoglycerophosphoglycerol, cytidine-5′-diphosphate-glycerols, glycosylglycerophospholipid, a glycerophosphoinositolglycan, 1,2-dialkyl-sn-glycero-3-Phosphate, 1,2-dialkyl-sn-glycero-3-phosphomethanol, 1,2-dialkyl-sn-glycero-3-phosphoethanol, 1,2-dialkyl-sn-glycero-3-phosphopropanol, and/or 1,2-dialkyl-sn-glycero-3-phosphobutanol.

A neutral lipid, when present in a lipoprotein system of the disclosure, can be any of a number of lipid species which exist either in an uncharged or neutral zwitterionic form at physiological pH. Such lipids include, for example, diacylphosphatidylcholine, diacylphosphatidylethanolamine, ceramide, sphingomyelin, dihydrosphingomyelin, cephalin, and cerebrosides. A lipid having a variety of acyl chain groups of varying chain length and degree of saturation is available or may be isolated or synthesized by well-known techniques. In some embodiments, a lipid containing saturated fatty acids with carbon chain lengths in the range of C₁₀ to C₂₀ is preferred. In another group of embodiments, a lipid with mono or diunsaturated fatty acids with carbon chain lengths in the range of C₁₀ to C₂₀ is used. Additionally, a lipid having a mixture of saturated and unsaturated fatty acid chains can be used. Preferably, the neutral lipid used is DOPE, DSPC, POPC, DPPC or any related phosphatidylcholine.

In some embodiments, the non-cationic lipid may be, but is not limited to, 1,2-di-O-octadecenyl-sn-glycero-3-phosphocholine (18:0 Diether PC), Acylcarnosine (AC), 1-hexadecyl-sn-glycero-3-phosphocholine (C16 Lyso PC), 1-oleoyl-2-hydroxy-sn-glycero-3-phosphocholine (18:1 Lyso PC), 1-myristoyl-2-hydroxy-sn-glycero-3-phosphocholine (14:0 Lyso PC), N-oleoyl-SPM (C18:1), N-lignoceryl SPM (C24:0), N-nervacyl C (C24:1), carbamoyl]cholesterol (Cet-P), cholesterol hemisuccinate (CHEMS), cholesterol (Chol), Cholesterol hemidodecanedicarboxylic acid (Chol-C12), 12-Cholesteryloxycarbonylaminododecanoicacid (Chol-C13N), Cholesterol hemioxalate (Chol-C2), Cholesterol hemimalonate (Chol-C3), N-(Cholesteryl-oxycarbonyl)glycine (Chol-C3N), Cholesterol hemiglutarate (Chol-C5), Cholesterol hemiadipate (Chol-C6), Cholesterol hemipimelate (Chol-C7), Cholesterol hemisuberate (Chol-C8), cardiolipid (CL), 1,2-bis(tricosa-10,12-diynoyl)-sn-glycero-3-phosphocholine (DC8-9PC), dicetylphosphate (DCP), dihexadecyl phosphate (DCP1), 1,2-Dipalmitoyglycerol-3-hemisuccinate (DGSucc), short-chain bis-n-heptadecanoyl phosphatidylcholine (DHPC), dihexadecoyl phosphoethanolamine (DHPE), 1,2-dilinoleoyl-sn-glycero-3-phosphocholine (DLPC), 1,2-dilauroyl-sn-glycero-3-PE (DLPE), Dimyristoyl glycerol hemisuccinate (DMGS), dimyristoylphosphatidylcholine (DMPC), dimyristoylphosphoethanolamine (DMPE), dimyristoyl phosphatidylglycerol (DMPG), dioleyloxy benzylalcohol (DOBA), 1,2-dioleoylglyceryl-3-hemisuccinate (DOGHEMS), N-[2-(2-{2-[2-(2,3-Bis-octadec-9-enyloxy-propoxy)-ethoxy]-ethoxy}-ethoxy)-ethyl]-3-(3,4,5-1rihydroxy-6-hydroxymethyl-1etrahydro-pyran-2-ylsulfanyl)-propionamide (DOGP4αMan), dioleoylphosphatidylcholine (DOPC), dioleoylphosphatidylethanolamine (DOPE), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenic phospholipid (DPhPE), dipalmitoylphosphatidylcholine (DPPC), dipalmitoyl phosphatidylethanolamine (DPPE), dipalmitoylphosphatidylglycerol (DPPG), dipalmitoyl phosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycerophosphocholine (DUPC), egg phosphatidylcholine (EPC), N-histidinyl cholesterol carbamate (HCChol), histamine distearoyl glycerol (HDSG), N-histidinyl cholesterol hemisuccinate (HistChol), 1,2-Dipalmitoylglycerol-hemisuccinate-Nα-Histidinyl-Hemisuccinate (HistSuccDG), N-(5′-hydroxy-3′-oxypentyl)-10-12-pentacosadiynamide (h-Pegi-PCDA), 2-[I-hexyloxyethyl]-2-devinylpyropheophorbide-a (HPPH), hydrogenated soybean phosphatidyl choline (HSPC), 1,2-Dipalmitoylglycerol-Oα-histidinyl-Nα-hemisuccinate (Isohistsucc DG), manno sialized dipalmitoyl phosphatidylethanolamine (ManDOG), 1,2-Dioleoyl-sn-Glycero-3-Phosphoethanolamine-N-[4-(p-maleimidomethyl)cyclohexane-carboxamide](MCC-PE), 1,2-diphytanoyl-sn-glycero-3-phosphoethanolamine (ME 16.0 PE), 1-myristoyl-2-hydroxy-sn-glycerophosphocholine (MHPC), athiol-reactivemaleimideheadgrouplipide.g.,1,2-dioleoyl-sn-glycero-3-phosphoethanolamine-N-[4-(p-maleimidophenyl)but-yramid (MPB-PE), Nervonic Acid (NA), sodium cholate (NaChol), 1,2-dioleoyl-sn-glycero-3-[phospho ethanolamine-N-dodecanoyl (NC12-DOPE), defined by synthesis example in International Patent Publication WO2008042973A2 (ND98), N-glutaryl phosphatidylethanolamine(s) of Formula1 (NG-PE) (the contents of which are herein incorporated by reference in their entirety), N-hydroxy sulfo succinimide (NHS-‘x’), “N-(co)-dicarboxylic acid-derivatized phosphatidyl ethanolamine encompassed by Formula1” (NωPE-‘x’), Oleic Acid (OA), 1-oleoyl-2-cholesterylhemisuccinoyl-sn-glycero-3-phosphocholine (OChemsPC), or phosphatidic acid (PA), phosphatidyl ethanolamine (PE), partially hydrogenated soy phosphatidyl choline (PHSPC), phosphatidyl inositol (PI), phosphotidylinositol-4-phosphate (PIP), palmitoyloleoylphosphatidylcholine (POPC), phosphatidylethanolamine (POPE), phosphatidylserine (PS), purified soy-derived mixture of phospholipids (SIOO), phosphatidylcholine (SM), 18-1-transPE,1-stearoyl-2-oleoyl-phosphatidyethanolamine (SOPE), soy bean phosphatidylcholine (SPC), sphingomyelins (SPM), alpha.alpha′-trehalose6,6′-dibehenate (TDB), 1,2-dielaidoyl-sn-glycero-3-phophoethanolamine (trans DOPE), ((23S,5R)-3-(bis(hexadecyloxy)methoxy)-5-(5-methyl-2,4-dioxo-3,4-dihydropyrimidin-1(2/-/)-yl)tetrahydrofuran-2-yl)methylmethylphosphate, 1,2-diarachidonoyl-sn-glycero-3-phosphocholine, 1,2-diarachidonoyl-sn-glycero-3-phosphoethanolamine, 1,2-didocosahexaenoyl-sn-glycero-3-phosphocholine, 1,2-didocosahexaenoyl-sn-glycero-3-phosphoethanolamine, 1,2-dilinolenoyl-sn-glycero-3-phosphocholine, 1,2-dilinolenoyl-sn-glycero-3-phosphoethanolamine, 1,2-dilinoleoyl-sn-glycero-3-phosphoethanolamine, 1,2-dioleyl-sn-glycero-3-phosphoethanolamine, 1,2-distearoyl-sn-glycero-3-phosphoethanolamine, 16-O-monomethyl PE,16-O-dimethyl PE, and dioleoyl phosphatidylethanolamine.

### Polymer-Conjugated Lipid

A lipid of the disclosure may be conjugated with a polymer. Conjugation with a polymer may be used to tune one or more properties of the lipoprotein systems of the disclosure to (i) reduce aggregation of the lipoprotein systems (ii) improve stability (iii) reduce immunogenicity and/or (iv) increase half-life of the lipoprotein systems of the disclosure.

In some embodiments, the polymer in the polymer-conjugated lipid may be or may include polyethylene glycol (PEG), polypropylene glycol (PPG), poly(2-oxazoline) (POZ), polyamide (ATTA), or any combination thereof.

In some embodiments, the polymer conjugated lipid may include a PEG derivative. As a non-limiting example, the PEG derivative may be a lipid-anchored PEG such as is C14-PEG1000, C14-PEG2000, C14-PEG3000, C14-PEG5000, C12-PEG1000, C12-PEG2000, C12-PEG3000, C12-PEG5000, C16-PEG1000, C16-PEG2000, C16-PEG3000, C16-PEG5000, C18-PEG1000, C18-PEG2000, C18-PEG3000, or C18-PEG5000.

In some embodiments, the lipid conjugated to the polymer may be selected from, but is not limited to, at least one of the cationic, non-cationic, or sterol lipids listed herein.

In some embodiments, the polymer conjugated lipid may be (PEG)-C-DOMG, (R)-2,3-bis(octadecyloxy)propyl-1-(methoxypoly(ethyleneglycol)2000)propylcarbamate, 1,2-distearoyl-sn-glycero-3-phosphoethanolamine-PEG2000, 14:0-PEG2KPE, DSPE-PEG-X, C14PEG, C14-PEG, C14-PEG 2000, C14PEG2000, C14-PEG2000, C14-PEG-DSPE200, C16 mPEG (polyethylene glycol) 2000 Ceramide, C16PEG, C18PEG, C18-PEG5000, C14PEG2000, C16PEG2000, C18PEG2000, C18PEG3000, C18PEG5000, C18PEG750, distearoyl-glycerol-polyethyleneglycol, DMG-PEG, DMG-PEG2000, DMG-PEGMA, DMPE-mPEG2000, DMPE-Peg, DMPE-PEG2000, DMPE-PEGMA, DOPE-PEG2000, DPG-PEGMA, DPPE-mPEG, DPPE-mPEG2000, DPPE-PEG, DPPE-PEG-2K, DSG-PEG5000, DSG-PEGMA, DSPE-PEG, DSPE-PEG2000, DSPE-PEG2000maleimide, DSPE-PEG2k, DSPE-PEG2K-NAG, DSPE-PEG5000, DSPE-PEG6000, DSPE-PEGMA, DSPE-PEGMA 500, Folate PEG-DSPE, HPEG-2K-LIPD, 1,2-distearoyl-sn-glycero-3-phosphoethanolamine-PEG2000, mDPPE-PEG2000, mPEG2000-DMG, mPEG2000-DMPE, mPEG2000-DPPE, MPEG2000-DSPE, MPEG-2000-DSPE, mPEG3000-DMPE, MPEG-DSPE, mPEG-PLA, Myrj52, N(Carbonyl-methoxypolyethylenglycol-2000)-1,2-distearoyl-sn-glycero3-phosphoethanolamine, PEG Click C10, PEG Click C12, PEG Click DMG C14, PEG DLPE C12, PEG DMPE C14, PEG DSPE C18, PEG(2k)-DMG, PEG200, PEG2000, PEG2000-C-DMA, PEG2000-DMG, PEG2000-DOMG, PEG2000-DSPE, PEG2000P, PEG2000-PE, PEG2k-C11, PEG2k-DMG, PEG400, PEG750-C-DMA, PEGA, PEG-cDMA, PEG-c-DMA, PEG-C-DMA, PEG-c-DMOG, PEG-C-DOMG, PEG-Ceramide C16, PEG-DMA, PEG-DMG, PEG-DMG 2000, PEG-DMG C14, PEG-DMG2000, PEG-DMPE, PEG-DMPE Na, PEG-DOMG, PEG-DPG, PEG-DSG, PEG-DSPE, PEG-OH DSPE C18, PEG-PAA, PEG-PE, PEG-S-DMG, PEG-S-DSG, R)-2,3-bis(octadecyloxy)propyl-1-(methoxypoly(ethyleneglycol)2000)propylcarbamate, or Siglec-1L-PEG-DSPE.

### Payloads

The lipoprotein system of the present disclosure may include at least one payload. As used herein, the term “payload” as used herein refers to any molecule that is associated with the lipoprotein system for delivery to a cell, tissue, subject or a biological system. Payloads of the present disclosure may include proteins, peptides, nucleic acids, lipids, lipid derivatives, glycolipids, carbohydrates, metabolites, metabolite derivatives, and/or small molecules.

The lipoprotein system may include one or more payloads (e.g., one, two, or three same or different payloads per recombinant lipoprotein). In some embodiments, a lipoprotein system may include 1 to 30 payloads that may be the same or different (e.g., 1 to 5, 1 to 10, 1 to 15, 1 to 20, 1 to 25, 5 to 10, 5 to 15, 5 to 20, 10 to 15, 10 to 20, 10 to 30, 15 to 20, 15 to 25, 15 to 30, 20 to 25, or 20 to 30). The lipoprotein system may have, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 same or different payloads.

The payloads may be present in one or more locations in or on the lipoprotein systems. As a non-limiting example, the payloads may be encapsulated in the core of the sphere in a lipoprotein system which assumes a spherical shape. The payloads may be sandwiched between the lipid bilayer of the lipoprotein system that assumes discoidal shape. The payloads may also penetrate the lipid membrane or be attached to the surface of the lipoprotein system.

In some embodiments, the payloads may be a hydrophobic molecule or a hydrophilic molecule or a combination thereof. Hydrophilic payloads may be enclosed in the interior of the lipoprotein systems. In some embodiments, hydrophobic payloads may be entrapped in the hydrocarbon chains of the lipids.

The payloads of the present disclosure may be amphipathic, e.g., sphingomyelin. The payloads need not be amphipathic, e.g., triglyceride.

In some embodiments, the payloads may interact directly or indirectly with any other component of the lipoprotein system. As a non-limiting example, the payloads may bind covalently or non-covalently to (a) a region of a polypeptide in the lipoprotein system, (b) a region of a lipid in the lipoprotein system, and/or (c) a linker that binds covalently or non-covalently to a polypeptide or a lipid in the lipoprotein system. In some embodiments, the payloads may not bind to any other component in the lipoprotein system.

The payloads of the disclosure may be charged or uncharged. In some embodiments, charged payloads may contribute a net charge (either positive or negative) of 1, 2, 3, or more than 3 to the lipoprotein system.

In some embodiments, the payloads of the present disclosure may be used for prophylactic, diagnostic, or therapeutic purposes. The payloads of the present disclosure may be prophylactics, diagnostics, or therapeutics for a disease that includes, without limitation, a neurological disease, an autoimmune disease, a cardiovascular disease, a vascular disease, a respiratory disease, a metabolic disease, a gastrointestinal disease, a genetic syndrome, a cancer, and a multisystem disease. In some embodiments, the payloads may target the lipoprotein system to a desired body region, such as a target organ, a target tissue, or a target cell, to provide the prophylactic, diagnostic, or therapeutic effect. In some embodiments, the payloads may provide immune evasion for the lipoprotein system. In some embodiments, the payloads may improve the solubility of the lipoprotein system in body fluids. Such features above may be part of, and or separate from, the prophylactic, diagnostic, and therapeutic effects of the payload. For example, a payload that assists the delivery of the lipoprotein system to desired body regions can also deliver a biological (e.g., therapeutic) effect to the desired body region.

In some embodiments, the compositions of the disclosure may include lipid components, drugs, proteins that are not membrane scaffold proteins, an/or diagnostic agents.

In some embodiments, the payload is a natural or synthetic bioactive small molecule. In some embodiments, the payload is a natural bioactive small molecule. In some embodiments, the payload is a combination of natural and synthetic bioactive small molecules.

In some embodiments, the payload is a macromolecule. In some embodiments, the payload is a macromolecule, such as a protein or peptide with a transmembrane domain immobilized within the lipoprotein systems. In some embodiments, the payload is a protein. In some embodiments, the payload is a peptide. In some embodiments, the payload is a protein with a transmembrane domain immobilized within the lipoprotein systems. In some embodiments, the payload is a peptide with a transmembrane domain immobilized within the lipoprotein systems. In some embodiments, the payload is a nucleic acid. In some embodiments, the payload is a nucleic acid incapsulated within the lipoprotein systems.

In some embodiments, the payload is an imaging agent or contrast agent. In some embodiments, the payload is an imaging agent. In some embodiments, the payload is a contrast agent. In some embodiments, the payload is a combination of an imaging agent and a contrast agent.

In some embodiments, the payload is an antioxidant. In some embodiments, the payload is an antioxidant (for example, vitamin E). In some embodiments, the payloads in, for example, vitamin E. In some embodiments, the payload is an antioxidant for the treatment of diseases or injuries marked by oxidative stress. In some embodiments, the payload is an antioxidant (for example, vitamin E) for the treatment of diseases or injuries marked by oxidative stress. In some embodiments, the payloads in, for example, vitamin E for the treatment of diseases or injuries marked by oxidative stress.

In some embodiments, the payload is an oxidizable lipid. In some embodiments, the payload is an oxidizable lipid (for example, a polyunsaturated fatty acid). In some embodiments, the payload is, for example, a polyunsaturated fatty acid. In some embodiments, the payload is an oxidizable lipid to promote oxidative stress and death. In some embodiments, the payload is an oxidizable lipid to promote oxidative stress and death (for example, in cancer cells). In some embodiments, the payload is an oxidizable lipid to promote oxidative stress and death in cancer cells. In some embodiments, the payload is an oxidizable lipid (for example, a polyunsaturated fatty acid) to promote oxidative stress and death. In some embodiments, the payload is an oxidizable lipid (for example, a polyunsaturated fatty acid) to promote oxidative stress and death (for example, in cancer cells). In some embodiments, the payload is an oxidizable lipid (for example, a polyunsaturated fatty acid) to promote oxidative stress and death in cancer cells. In some embodiments, the payloads i, for example, a polyunsaturated fatty acid to promote oxidative stress and death. In some embodiments, the payload is, for example, a polyunsaturated fatty acid to promote oxidative stress and death (for example, in cancer cells). In some embodiments, the payload is, for example, a polyunsaturated fatty acid to promote oxidative stress and death in cancer cells.

In some embodiments, the payload is a lipid that is depleted with age or due to an inborn error of metabolism. In some embodiments, the payload is a lipid that is depleted with age. In some embodiments, the payload is a lipid that is depleted due to an inborn error of metabolism.

In some embodiments, the payload is a lipid or lipid precursor which promotes issue repair after injury. In some embodiments, the payload is a lipid which promotes issue repair after injury. In some embodiments, the payload is a lipid precursor which promotes issue repair after injury. In some embodiments, the payload facilitates the subsequent removal of a lipid. In some embodiments, the payload is any combinations of, for example, a natural bioactive small molecule, a synthetic bioactive small molecule, a macromolecule, a protein, a peptide, a protein with a transmembrane domain, a peptide with a transmembrane domain, a nucleic acid, a nucleic acid incapsulated, an imaging agent, a contrast agent, an antioxidant, an oxidizable lipid, a chemically modified lipid, a lipid depleted, a lipid which promotes issue repair after injury, a lipid precursor which promotes issue repair after injury, as described herein.

### Protein and Peptide Payloads

The payload of the lipoprotein system disclosed herein may include one or more proteins or peptides as payloads. Any naturally occurring protein may be a payload of the disclosure. The protein payload or peptide payload may be a mammalian protein or peptide. In some embodiments, the protein payload or peptide payload may be from a mammal such as, but not limited to, human, mouse, rat, dog, pig, goat, llama, or sheep.

Proteins and peptides used as the payloads of the disclosure may be utilized to treat conditions or diseases associated with central nervous system, blood, the cardiovascular system, poisoning (including antivenoms), dermatology, endocrinology, genetic, genitourinary, gastrointestinal, musculoskeletal, oncology, and immunology, respiratory, sensory, and anti-infective.

In some embodiments, the protein or peptide payload may be classified by a cellular location. In some embodiments, the protein payload may be a nuclear protein payload, a cytoplasmic protein payload, a membrane protein payload, a lysosomal protein payload, a mitochondrial protein payload, an endoplasmic reticulum protein payload, a cytoskeletal protein payload, an intracellular membrane surface protein payload, a transmembrane protein payload, and/or the extracellular matrix protein payload. In some embodiments, the peptide payload may be a nuclear peptide payload, a cytoplasmic peptide payload, a membrane peptide payload, a lysosomal peptide payload, a mitochondrial peptide payload, an endoplasmic reticulum peptide payload, a cytoskeletal peptide payload, an intracellular membrane surface peptide payload, a transmembrane peptide payload, and/or the extracellular matrix peptide payload.

In some embodiments, payloads of the lipoprotein system may also be a fusion protein. The fusion protein may be created by operably linking a charged protein to a therapeutic protein. In some embodiments, the fusion protein may contain a region of an immunoglobulin chain fused to a protein or a peptide described herein (see U.S. Pat. No. 8,053,569, the contents of which are incorporated herein by reference in their entirety). In some embodiments of the disclosure, payloads of the lipoprotein system may include a protein of the plasma membrane. In some embodiments of the disclosure, payloads of the lipoprotein system may include a cytoplasmic or cytoskeletal protein. In some embodiments of the disclosure, payloads of the lipoprotein system may include an intracellular membrane bound protein. In some embodiments of the disclosure, payloads of the lipoprotein system may include a nuclear protein. In some embodiments of the disclosure, payloads of the lipoprotein system may include a protein associated with human disease. In some embodiments of the disclosure, payloads of the lipoprotein system may include a protein with a presently unknown therapeutic function. In some embodiments, payloads may include any coding or non-coding gene or any protein or fragment thereof. In some embodiments, the payloads of the disclosure may include lipoprotein receptors (e.g., LDLR, LRP, and SR—B1), enzymes (e.g., LCAT and lipases), transfer proteins (e.g., PLTP and CETP), and/or ABC transporters (e.g., ABCA1 and ABCG1).

### Antigens

In some embodiments, payloads of the lipoprotein system may include at least one antigen. As used herein, “antigen” embraces the meanings and uses that a person of ordinary skill in the art would understand the term to embrace, and additionally refers to any molecule that results in an alteration in the reactivity of a subject's immune system when the molecule is introduced or produced by the subject. Antigens may be proteins, peptides, and/or peptide fragments. In some embodiments, antigens are non-amino acid-based molecules.

In some embodiments, the antigens of the present disclosure may be derived from a specific subcellular location. In some embodiments, sub cellular location from which the antigen is derived may be the plasma membrane, the cell surface, the nucleus, the cytosol, the lysosome, the endosome, the mitochondria, the peroxisome, the Golgi body, the endoplasmic reticulum or any other organelle within the cell. In some embodiments, the cell may be a eukaryotic or a prokaryotic cell.

In some embodiments, antigens of the present disclosure, may be classified based on the tissues from which the antigen originates. Such antigens may be a nervous tissue antigen, wherein the antigen is specific to the neurological tissue such as the brain, spinal cord, the central nervous system, the peripheral nervous system, including the sympathetic and parasympathetic nervous system. Such antigens may also originate from neuronal cell types such as Schwann cells, the axon, the motor or the ganglioside neuron, the glial cells, the astrocytes, progenitor cells, oligodendrocytes.

In some embodiments, the antigen may be a connective tissue antigen, implying that the antigen may be expressed by cells that bind other cells, and organs of the body together. Connective tissue antigens may be derived from loose connective tissues such as areolar connective tissue, adipose tissue, reticular tissue; dense connective tissue such as dense regular connective tissue or dense irregular connective tissue; or special connective tissue such as cartilage, bone and blood.

In other embodiments, the antigen is a muscle antigen. Such muscle antigens may be derived from skeletal (voluntary) muscle tissue, smooth muscle tissue and/or cardiac muscle tissue.

The antigen of the present disclosure may be an epithelial antigen derived from the epithelium that covers the exterior surface of the body and lines the internal cavities and passageways as well as forms certain glands. Antigens of the present disclosure may be derived from cuboidal epithelium, squamous epithelium and/or columnar epithelium.

In some embodiments, the antigen is a tumor protein, a bacterial protein, a viral protein, a fungal protein, a protozoan protein, a parasite protein, or any fragments or portions thereof.

In some embodiments, the tumor protein is a protein that is associated with a cancer. The cancer includes but is not limited to brain cancer, lung cancer, liver cancer, kidney cancer, bone cancer, skin cancer, renal cancer, urinary bladder cancer, prostate cancer, uterine cancer, breast cancer, cervical cancer, ovarian cancer, stomach cancer, colon cancer, rectal cancer, oral cavity cancer, pharynx cancer, pancreatic cancer, thyroid cancer, head and neck cancer, hematopoietic cancer, leukemia, lymphoma, melanoma, and sarcoma.

In some embodiments, the viral protein may be an adenovirus protein, an arterivirus protein, an arenavirus protein, a bornavirus protein, a bunyavirus protein, a flavivirus protein, a filovirus protein, a herpesvirus protein, a hepadnavirus protein, an orthomyxovirus protein, a picornavirus protein, a paramyxovirus protein, a polyomavirus protein, a papillomavirus protein, a poxvirus protein, a rhabdovirus protein, a reovirus protein, a retrovirus protein, or a togavirus protein.

In some embodiments, the antigen is a cytomegalovirus (CMV) protein, an Ebola Virus (EBOV) protein, a hepatitis C virus (HCV) protein, a Campylobacter protein, an influenza virus protein, a respiratory syncytial virus (RSV) protein, a human immunodeficiency virus (HIV) protein, a Lassa fever virus (LFV) protein, a Mycobacterium protein, a Bacillus protein, a Yersinia protein, a Streptococcus protein, a Shigella protein, a Salmonella protein, a Pseudomonas protein, a Plasmodium protein, or a Toxoplasma protein.

In some embodiments, the antigen includes, consists of, or consists essentially of a protein or peptide or a fragment, portion, variant, homologue, derivative, or subsequence thereof that is associated with a neurological disease or condition. The protein or peptide may be a viral protein derived from a virus such as, but not limited to Herpes simplex virus (HSV), Herpes simplex virus-1 (HSV-1), Roseolovirus, Human herpesvirus 6 (HHV-6), Epstein-Barr virus (EBV or HHV-4), cytomegalovirus (CMV), varicella zoster virus (VZV, also referred to as Human alpha herpes virus 3, HHV-3), JC virus, Borna disease virus, Influenza virus, H3N2, H1N1, H2N2, H5N1, Measles, Rabies virus, West Nile Virus, Japanese encephalitis B virus, St. Louis virus, Poliovirus, Echo virus, Coxsackie virus, respiratory syncytial virus (RSV), Coronavirus, SARS-CoV-2, OC43, 229E, enteroviruses polioviruses (PV), coxsackieviruses (CV), echovirus, and human rhinoviruses (HRV), human metapneumovirus (hMPV), Enterovirus A71 (EV-A71), Enterovirus D68 (EV-D68), human immunodeficiency virus (HIV), and coronavirus.

### Antibodies

In some embodiments, antibodies or antibody-based compositions may be utilized as a payload. As used herein, the term “antibody” is referred to in the broadest sense and covers various embodiments including, but not limited to, monoclonal antibodies, polyclonal antibodies, multispecific antibodies (e.g., bispecific antibodies formed from at least two intact antibodies), and antibody fragments (e.g., diabodies) so long as they exhibit a desired biological activity (e.g., “functional”). Antibodies may include one or more modifications (including, but not limited to the addition of sugar moieties, fluorescent moieties, chemical tags, etc.).

Payloads of the present disclosure may include polypeptides that form or function as any antibody, including antibodies that are known in the art and/or antibodies that are commercially available. Further, polypeptides of the disclosure may include fragments of such antibodies or antibodies that have been developed to include one or more of such fragments (e.g., variable domains or complementarity determining regions (CDRs)).

In some embodiments, payloads may include antibodies, antibody fragments, or components of any of those described in U.S. Pat. No. 11,046,755 related to tau oligomers; (the contents of which are incorporated by reference in their entirety).

In some embodiments, antibody fragments used as payloads of the disclosure may include antigen binding regions from intact antibodies. Antibody fragments include, but are not limited to, Fab, Fab′, F(ab′)2, and Fv fragments; diabodies; linear antibodies; single-chain antibody molecules; and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)2 fragment that has two antigen-binding sites and is still capable of crosslinking antigen. A payload of the present disclosure may include one or more of these fragments. For the purposes herein, an “antibody” may include a heavy and light variable domain as well as an Fc region.

In some embodiments, payloads of the disclosure may include antibody variable domains. As used herein, the term “variable domain” refers to specific antibody domains found on both the antibody heavy and light chains that differ extensively in sequence among antibodies and are used in the binding and specificity of each particular antibody for its particular antigen. Variable domains include hypervariable regions. As used herein, the term “hypervariable region” refers to a region within a variable domain comprising amino acid residues responsible for antigen binding. The amino acids present within the hypervariable regions determine the structure of the complementarity determining regions (CDRs) that become part of the antigen-binding site of the antibody. As used herein, the term “CDR” refers to a region of an antibody comprising a structure that is complimentary to its target antigen or epitope.

In some embodiments, the payloads may be a “single chain Fv” or “scFv” which refers to a fusion protein of VH and VL antibody domains, wherein these domains are linked together into a single polypeptide chain by a flexible peptide linker. In some embodiments, the Fv polypeptide linker enables the scFv to form the desired structure for antigen binding.

In some embodiments, the payloads may be a bispecific antibody. As used herein, the term “bispecific antibody” refers to an antibody capable of binding two different antigens. Such antibodies typically include regions from at least two different antibodies. Bispecific antibodies may include any of those described in Riethmuller, G. 2012. Cancer Immunity. 12:12-18, Marvin, J. S. et al., 2005. Acta Pharmacologica Sinica. 26(6):649-58 and Schaefer, W. et al., 2011. PNAS. 108(27):11187-92, the contents of each of which are herein incorporated by reference in their entirety.

In some embodiments, the payloads of the disclosure may be a diabody, an intrabody or a monoclonal antibody. As used herein, the term “diabody” refers to a small antibody fragment with two antigen-binding sites. Diabodies include a heavy chain variable domain VH connected to a light chain variable domain VL in the same polypeptide chain. The term “intrabody” refers to a form of antibody that is not secreted from a cell in which it is produced, but instead targets one or more intracellular proteins. Intrabodies may be used to affect a multitude of cellular processes including, but not limited to intracellular trafficking, transcription, translation, metabolic processes, proliferative signaling and cell division. As used herein, the term “monoclonal antibody” refers to an antibody obtained from a population of substantially homogeneous cells (or clones), i.e., the individual antibodies comprising the population are identical and/or bind the same epitope, except for possible variants that may arise during production of the monoclonal antibodies, such variants generally being present in minor amounts. In contrast to polyclonal antibody preparations that typically include different antibodies directed against different determinants (epitopes), each monoclonal antibody is directed against a single determinant on the antigen.

In some embodiments, payloads of the disclosure may include antibodies that bind more than one epitope. As used herein, the terms “multibody” or “multispecific antibody” refer to an antibody wherein two or more variable regions bind to different epitopes. The epitopes may be on the same or different targets. In certain embodiments, a multi-specific antibody is a “bispecific antibody,” which recognizes two different epitopes on the same or different antigens.

In some embodiments, payloads of the disclosure may include bispecific antibodies. Bispecific antibodies are capable of binding two different antigens. Such antibodies typically include antigen-binding regions from at least two different antibodies. For example, a bispecific monoclonal antibody (BsMAb, BsAb) is an artificial protein composed of fragments of two different monoclonal antibodies, thus allowing the BsAb to bind to two different types of antigens.

In some embodiments, payloads may include antibodies comprising a single antigen-binding domain. These molecules are extremely small, with molecular weights approximately one-tenth of those observed for full-sized mAbs. Further antibodies may include “nanobodies” derived from the antigen-binding variable heavy chain regions (VHHs) of heavy chain antibodies found in camels and llamas, which lack light chains (Nelson, A. L., MAbs.2010. January-February; 2(1):77-83).

In some embodiments, payloads of this disclosure may include antibodies selectively binding to surface marker proteins of senescent cells. For example, the antibodies may selectively bind to proteins that are in misfolded conformation. The binding antibodies may reduce the number of senescent cells and be used to treat age-related conditions, such as, but not limited to, Alzheimer's disease, Parkinson's Disease, cardiovascular disease, emphysema, sarcopenia, and tumorigenesis as well as conditions more cosmetic in nature such as signs of skin aging including wrinkling, sagging, discoloration, age-related tissue dysfunction, tumor formation, and other age-related conditions.

### Enzymes

In some embodiments, the payloads of the lipoprotein system may include at least one enzyme. Enzymes, known as biological catalysts, are proteins produced by or derived from a living organism which can cause, accelerate or inhibit a specific biochemical reaction. The lipoprotein systems of the disclosure may provide a therapeutic delivery approach for enzyme payloads.

Neuroprotective enzymes may be used as the payloads of the lipoprotein system to treat neurodegenerative disorders. As a non-limiting example of such enzyme, Tripeptidyl peptidase 1 (TPP1) can cut the amyloid-beta (Ap) plaques into pieces, leading to a new approach to treat Alzheimer's disease (Sol6-Domenecha et al. Proc Natl Acad Sci USA. 2018 Feb. 13; 115(7):1493-1498; the contents of which are herein incorporated by reference in its entirety). In some embodiments, the payloads may be enzyme aromatase which is produced during different types of brain injury to catalyze the conversion of testosterone and other C19 steroids to the neuroprotective estradiol. As another example, the payloads may be members of the cytosolic carboxypeptidase (CCP) family, CCP1, CCP4, and CCP6, which can play a positive role in preventing neurodegenerative disorders by catalyzing deglutamylation of tubulin since the microtubule hyper glutamylation is directly linked to neurodegeneration. In other aspects, the payloads may be cytochrome P450 2D6 (CYP2D6), an enzyme highly expressed in the CNS. This enzyme is 40% lower in the frontal cortex, cerebellum, and the hippocampus of the Parkinson's disease patients than that of the control. In some embodiments, the payloads may be LRRK2. A multidomain protein enzyme, LRRK2, is hyperactivated in the Parkinson's disease patients. PPM1H phosphatase counteracts LRRK2 signaling and thus may offer new therapy to prevent or treat Parkinson's disease.

Other non-limiting examples of enzyme payloads of the lipoprotein system include aspartylglucosamininidase, Palmitoyl protein thioesterase, tripeptidyl peptidase, α-Galactosidase, α-Fucosidase, Protective protein/cathepsin A, β-Glucosidase, Galactosylceramidase, β-Galactosidase, α-Mannosidase, Arylsulfatase A, α-L-Iduronidase, iduronate sulfatase, Heparin Sulfatase, α-N-acetylglucosaminidase, acetyl-CoA alpha glucosaminide acetyltransferase, N-acetylglucosamine-6-sulfate sulfatase, N-acetylgalactosamine 6-sulfatase, hyaluronidase, hexosaminidase A, Arylsulfatase B, β-Glucuronidase, α-neuraminidase, GlcNAc-1-phosphotransferase, N-acetylglucosamine-1-phosphotransferase, Acid sphingomyelinase, α-Glucosidase, β-Hexosaminidase A and B, α-N-acetylgalactosaminidase, β-Hexosaminidase A, Acid lipase, Tyrosine hydroxylase, and/or lecithin-cholesterol acyltransferase (LCAT).

### Growth Factors and Hormones

In some embodiments, the payloads of the lipoprotein system may include at least one growth factor. As used herein, “growth factor” refers to any proteins or peptides that stimulate or promote the growth of tissues, cell proliferation, or cell differentiation. The growth factor may be naturally-occurring growth factors or any variants or fragments thereof, or any synthetic peptides or proteins that mirror the structure, function, or both structure and function of naturally-occurring growth factors. In some embodiments, a growth factor may be a cytokine.

Some growth factors can prevent cell death in degenerative processes and enhance the growth and function of affected neurons in the degenerating human brain. Therefore, they can act as neurotherapeutic agents for the treatment, prevention, or delay of progression of neurodegenerative diseases or neurodegeneration-induced diseases (see Neural Regeneration Research. 2017; 12(4):549-557.) As a non-limiting example, the payload of the disclosure may be BDNF. In experimental models, the intracerebral administration of brain-derived neurotrophic factor (BDNF) was found to be neuroprotective in stroke, global brain ischemia, prion diseases, Alzheimer's disease (AD), Parkinson's disease (PD), Huntington's disease (HD), or amyotrophic lateral sclerosis (ALS), the direct intracerebral injection of BDNF is neuroprotective (see U.S. Pat. No. 8,053,569; the contents of which are herein incorporated by reference in its entirety).

In some embodiments, the lipoprotein system of the disclosure may be used to deliver the neurotherapeutic growth factors into the brain, which facilitates their neurotherapeutic or neuroprotective roles in the CNS. In some embodiments, the lipoprotein system crosses the BBB by transcytosis. In some embodiments, the transcytosis of the lipoprotein system crossing the BBB is mediated by scavenger receptor BI (SR—BI) (Houlton, et al. 2019; Front Physiol. 2017; 8: 841; the contents of which are herein incorporated by reference in its entirety).

In some embodiments, a growth factor payload may improve the permeability of the BBB. By administering human VEGF165 and subsequently Evans blue to Kunming mice, Evans blue fluorescence intensity was found to be higher in the parenchyma and lower in the brain vasculature of VEGF-treated mice compared to controls (Jiang et al. 2014 PLoS One. 2014 Feb. 14; 9(2):e86407; the contents of which are herein incorporated by reference in their entirety). Growth factors capable of improving the permeability of the BBB can be combined with the lipoprotein system of the disclosure in administration to subjects either in risk of or suffering neurodegenerative diseases, which can enhance the penetration of the lipoprotein systems into the brain and the CNS and thus its therapeutic effects.

In some embodiments, the payloads of the lipoprotein system may include at least one hormone. As used herein, “hormone” refers to any organic substance secreted by animals and plants that regulate physiological and behavioral activities such as respiration, metabolism, digestion, sensory perception, sleep, excretion, lactation, stress induction, development, movement, reproduction, and mood manipulation in animals, and every aspect of growth and development in plants. In the human body, hormones include peptides, amino acid derivatives, steroids, and eicosanoids. In some embodiments, the payload is a peptide hormone. The peptide hormone used as a payload of the lipoprotein system can be naturally-occurring hormones or any variants or fragments thereof, or any synthetic peptides or proteins that mirror the structures, functions, or both structures and functions of naturally-occurring peptide hormones. In some embodiments, the peptide hormones may refer to neuropeptides.

In some embodiments, the payload of the disclosure may include, without limitation, Adiponectin (Acrp30), Adipose-derived hormone, Adrenocorticotropic hormone (ACTH, or corticotropin), Agouti signaling peptide, Allatostatin, Amylin (or Islet Amyloid Polypeptide, IAPP), Angiotensinogen, Angiotensin (AGT), Antidiuretic hormone (ADH, or vasopressin), Atrial natriuretic peptide (ANP, or atriopeptin), Adrenomedullin (AM), Angiopoietin (Ang), Autocrine motility factor, Artemin, alpha-melanocyte-stimulating hormone, Brain natriuretic peptide (BNP), Bone morphogenetic proteins (BMPs), Bomben-like peptide, Big gastrin (G34), Betatrophin, Bradykinin, Brain-derived neurotrophic factor (BDNF), Cardiotrophin-1 (CT-1), Calcitonin (CT), Cholecystokinin (CCK), Corticotrophin-releasing hormone (CRH), Cortistatin (CORT), Cosyntrophin, Ciliary neurotrophic factor (CNTF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Enkephalin, Endothelin, Enteroglucagon, Fetal Bovine Somatotrophin (FBS), Fibroblast growth factor 11 (FGF11), Fibroblast growth factor 2 (FGF2), Fibroblast growth factor 3 (FGF3), Fibroblast growth factor 4 (FGF4), Fibroblast growth factor 5 (FGF5), Fibroblast growth factor 6(FGF6), Fibroblast growth factor 7 (FGF7), Fibroblast growth factor 8 (FGF8), Fibroblast growth factor 9 (FGF9), Fibroblast growth factor 10 (FGF10), Fibroblast growth factor 11 (FGF11), Fibroblast growth factor 12 (FGF12), Fibroblast growth factor 13 (FGF13), Fibroblast growth factor 14 (FGF14), Fibroblast growth factor 15 (FGF15), Fibroblast growth factor 16 (FGF16), Fibroblast growth factor 17 (FGF17), Fibroblast growth factor 18 (FGF18), Fibroblast growth factor 19 (FGF19), Fibroblast growth factor 20 (FGF20), Fibroblast growth factor 21 (FGF21), Fibroblast growth factor 22 (FGF22), Fibroblast growth factor 23 (FGF23), Fibronectin type III domain-containing protein 5 (FNDC5), Follicle-stimulating hormone (FSH), Galanin (GAL), Gastric inhibitory polypeptide (GIP), Gastrin (GAS), Granulocyte colony-stimulating factor (G-CSF), Granulocyte macrophage colony-stimulating factor (GM-CSF), Growth differentiation factor-9 (GDF9), Ghrelin, Glucagon (GCG), Glucagon-like peptide-1 (GLP1), Gonadotrophin, Gonadotropin-releasing hormone (GnRH), Growth hormone (GH), Growth hormone releasing hormone (GHRH), Glial cell line-derived neurotrophic factor (GDNF), Hepatocyte growth factor (HGF), Hepatoma-derived growth factor (HDGF), Hepcidin (HAMP), Human chorionic gonadotrophin (hCG), Human placental lactogen (HPL), Insulin (INS), Insulin-like growth factor-1 (IGF-1), Insulin-like growth factor-2 (IGF-2), Interleukin-1 (IL-1), IL-2, IL-3, IL-4, IL-5, IL-6, IL-7, Incretin, Inhibin, Keratinocyte growth factor (KGF), Leukemia inhibitory factor (LIF), Interleukin-6 (IL-6), Leptin (LEP), Lipotropin (LPH), Luteinizing hormone (LH), Leptin (LEP), Little gastrin, Liraglutide, Luteinizing hormone, Macrophage colony-stimulating factor (m-CSF), Macrophage-stimulating protein (MSP), Migration-stimulating factor (MSF), Myostatin (GDF-8), Midkine (MDK), Melanocortin, Melanocyte stimulating hormone (MSH), Minigastrin, Motilin (MLN), Nerve growth factor (NGF), Neurotrophin-3 (NT-3), Neurotrophin-4 (NT-4), Neuregulin 1 (NRG1), Neuregulin 2 (NRG2), Neuregulin 3 (NRG3), Neuregulin 4 (NRG4), Netrin, Neurturin, Neuropeptide Y, NPH insulin, Orexin, Obestatin, Osteocalcin (OCN), Oxytocin (OXT), Pancreatic hormone, Parathyroid hormone (PTH), peptide YY, Persephin, Pituitary adenylate cyclase-activating peptide (PACAP), Placental growth factor (PGF), Platelet-derived growth factor (PDGF), Pleiotrophin (PTN), Prolactin (PRL), Prolactin-releasing hormone (PRLH), preprohormone, Renalase (RNLS), Relaxin (RLN), Renin, Saposin, Salcatonin, Semophorin, Somatostatin (or growth hormone-inhibiting hormone, GHIH), Secretin (SCT), Substance P, Sincalide, Transforming growth factor alpha (TGF-α), Transforming growth factor beta (TGF-β), T-cell growth factor (TCGF), Thrombopoietin (TPO), Thyroid-stimulating hormone (TSH, or thyrotropin), Thyrotropin-releasing hormone (TRH), Tumor necrosis factor-alpha (TNF-α), Teleost leptins, Temporin, Tesamorelin, Urocortin, Uroguanylin (UGN), Vasoactive intestinal peptide (VIP), Vascular endothelial growth factor (VEGF), VGF and Vitellogenin.

In some embodiments, the payload of the lipoprotein system of the disclosure is a neurotherapeutic growth factor or a fragment or variant thereof, or any nucleotides encoding neurotherapeutic growth factors or fragments or variants thereof. In some embodiments, the payloads of the lipoprotein system of the disclosure may be any synthetic peptides or proteins that mirror the structure, function, or both structure and function of said neurotherapeutic growth factors. Non-limiting examples of said neurotherapeutic growth factors include Brain-derived neurotrophic factor (BDNF), Nerve growth factor (NGF), Neurotrophin-3 (NT-3), Neurotrophin-4 (NT-4), Glial cell-derived neurotrophic factor (GDNF), Transforming growth factor alpha (TGF-alpha), Insulin-like growth factor 1 (IGF-1), Vascular endothelial growth factor (VEGF), Ciliary neurotrophic factor (CNTF), or myostatin.

In some embodiments, the payload of the lipoprotein system of the disclosure may be one or more neurotherapeutic growth factors such as, but not limited to, NGF, BDNF, and NT-3. In some embodiments, the payload of the lipoprotein systems of the disclosure is BDNF.

In some embodiments, the payload of the lipoprotein system of the disclosure may be polynucleotides that encode one or more neurotherapeutic growth factors selected from NGF, BDNF, and NT-3.

In some embodiments, the peptide hormones used as payloads may be neuropeptides. In some embodiments, the payloads of the lipoprotein system of the disclosure may include neuropeptides. Neuropeptide as used here refers to any peptide that is synthesized and released by neurons and modulates neural activity for e.g., by binding to G protein-coupled receptors (CPCRs).

In some embodiments, a payload of the disclosure is a neuropeptide, any variant thereof, any nucleotides encoding the neuropeptides, or any synthetic peptides or proteins that mirror the structure, function, or both structure and function of the neuropeptides. Non-limiting examples of neuropeptides are Neurokinin, Neurotrophin, CRH, Substance P, Neuropeptide Y, Dynorphins, Somatostatin, Leptin, Cocaine- and amphetamine-regulated transcript (CART), and/or Ghrelin.

### Protein-Ligand Complexes

In some embodiments, the payload of the lipoprotein system may include at least one protein-ligand complex. A protein-ligand complex is a complex of protein bound with ligand through molecular recognition, where ligand means any molecule that binds to the protein with high affinity and specificity, such as a protein, peptide, nucleic acid, lipid, lipid derivative, sterol, steroid, metabolite, metabolite derivative or small molecule. In some embodiments, said complex may further undergo chelation to include metal atom.

### Nucleic Acids

Payloads of the lipoprotein system may be nucleic acid-based (also herein referred to as polynucleotide). The term “nucleic acid,” in its broadest sense, includes any compound and/or substance that includes a polymer of nucleotides, e.g., linked nucleosides. These polymers are often referred to as polynucleotides. Exemplary nucleic acids or polynucleotides used as payloads of the disclosure include, but are not limited to, deoxyribonucleic acids (DNAs), ribonucleic acids (RNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof. While not wishing to be bound by theory, the lipoprotein system of the disclosure may protect the nucleic acid payloads from nuclease degradation in physiological fluids and/or help nucleic acid payloads evade the interception by the immune system, reach target organs, tissues, or cells, and escape endosomes to reach the cytoplasm.

As a non-limiting example, DNA used as payloads of the lipoprotein system may be a double-stranded DNA (dsDNA), a single-stranded DNA (ssDNA), a plasmid, a complementary DNA (cDNA), a DNA analog, and a fragment thereof.

As a non-limiting example, RNA used as payloads of the lipoprotein system may be a messenger RNA (mRNA), a non-coding RNA (ncRNA), a long non-coding RNA (lncRNA), a double-stranded RNA (dsRNA), a single-stranded RNA, a circular RNA (circRNA), a ribozyme, an RNA interference (RNAi) agent like but not limited to miRNA and siRNA, an RNA activation agent like but not limited to small activating RNA (saRNA), a guide RNA (gRNA), an RNA analog, and a fragment thereof.

In some embodiments, the lipoprotein system of the disclosure may further include at least one antisense oligonucleotide (ASO). The ASO may include a modification at a position in oligonucleoside (e.g., a modification at the ribose portion of the nucleoside, a modification in the internucleoside linkage, etc.). The ASO may include at least 10 nucleosides (e.g., at least 10 to 50 nucleosides, at least 20 to 50 nucleosides, at least 30 to 50 nucleosides, at least 40 to 50 nucleosides, at least 50 nucleosides, etc.).

In some embodiments, the lipoprotein system of the disclosure may include an RNA analog such as a synthetic analog of double-stranded RNA, polyinosinic-polycytidylic acid (poly(I:C).

In some embodiments, payloads of the lipoprotein systems may be a messenger RNA (mRNA). The mRNA may or may not be chemically modified. In some embodiments, polynucleotides used as payloads of the lipoprotein system may encode a protein or peptide payload.

In some embodiments, payloads of the lipoprotein system of the disclosure may include a replicon mRNA. As used here, the terms “replicon mRNA,” “replicon RNA,” “self-replicating RNA,” or a “self-amplifying RNA” refer to a polynucleotide derived from alphaviruses which is a positive-strand RNA and which encodes RNA-dependent RNA polymerases that simultaneously express encoded proteins or peptide payloads and self-amplify the polynucleotide on entry into the cytoplasm. Exogenous RNAs degrade rapidly in recipient cells, limiting their therapeutic utilities. Even with biological modifications (such as enzymatic capping and polyadenylation) and incorporation of chemically modified nucleotides, exogenous RNA often remains active in cells for only several days, making them unsuitable for long-term gene therapy. The self-replicating nature of replicon mRNA provides an alternative approach. As a non-limiting example, the product of interest is a protein or peptide payload.

In some embodiments, the lipoprotein system of the disclosure may further include at least one replicon mRNA for expressing the payloads in vitro, in vivo, in situ or ex vivo. In some embodiments, the lipoprotein system of the disclosure may encapsulate at least one replicon mRNA for expressing the product of interest in vitro, in vivo, in situ or ex vivo.

In some embodiments, payloads of the lipoprotein system of the disclosure may be RNA interference (RNAi) agents. RNA interference methods using RNAi agents may be used to disrupt the expression of a gene or polynucleotide of interest.

While not wishing to be bound by theory, RNAi agents may be a) RNA:RNA hybrids comprising both an RNA sense and an RNA antisense strand, b) DNA sense: RNA antisense hybrids, c) RNA sense: DNA antisense hybrids, or d) DNA: DNA hybrids. As used herein, RNAi agents encompass any and all molecules capable of inducing an RNAi response in cells, including, but not limited to, double-stranded oligonucleotides comprising two separate-strands, i.e., a sense strand and an antisense strand, e.g., small interfering RNA (siRNA); double-stranded oligonucleotide comprising two separate strands that are linked together by non-nucleotide linker; oligonucleotides comprising a hairpin loop of complementary sequences, which forms a double-stranded region, e.g., short hairpin RNA (shRNA), and expression vectors that express one or more polynucleotides capable of forming a double-stranded polynucleotide alone or in combination with another polynucleotide.

In some embodiments, the lipoprotein system of the disclosure can be engineered to include payloads such as therapeutic RNAi agents including, without limitation, microRNA (miRNA), small interfering RNA (siRNA), short hairpin RNA (shRNA), and piwi-interacting RNA (piRNA).

In some embodiments, payloads of the lipoprotein system of the disclosure may be a guide RNA (gRNA). As used herein, the term “guide RNA” refers to an RNA molecule that is used in conjunction with a CRISPR associated system and directs an RNA-guided DNA binding agent to a target sequence. As a non-limiting example, the RNA-guided DNA binding agent is a Cas cleavage, a Cas nickase, or an inactivated form thereof (e.g., dCas molecules like dCas9 and dCas12a).

### Lipids

In some embodiments, the payload of the disclosure may be a lipid payload. Any lipid described herein may be used as a lipid payload.

In some embodiments, the lipid payload may be a fatty acid payload, a steroid payloads or a sterol ester payload. In some embodiments, the fatty acid payload may be a free fatty acid, a wax, a triacylglycerol, a glycerophospholipid, a sphingolipid, or a glycerosphingolipid. In some embodiments, the lipid payload may be a cationic lipid, a fusogenic lipid, an anionic lipid, a neutral lipid, or any combination thereof.

In some embodiments, the lipid payload may be a steroid. As a non-limiting example, the steroid may be cholesterol and/or Vitamin D. The lipid payload may be a steroid hormone. The steroid hormone payloads may be naturally-occurring or synthetic hormones or any variants thereof. In one embodiment, the steroid hormone may be estrogen.

The lipid payload may be a glycerophospholipid such as, but not limited to, phosphatidic acid, phosphatidylethanolamine, a phosphatidylcholine, a phosphatidylserine, and/or phosphatidylglycerol. As a non-limiting example, the lipid payload may be cardiolipin.

In some embodiments, the lipid payload may be a sphingolipid. The sphingolipid may be a ceramide, a cerebroside, a sphingomyelin, or a ganglioside. In one embodiment, the lipid payload may be linolenic acid (e.g., alpha-linolenic acid (ALA) or a gamma-linolenic acid (GLA).

### Carbohydrates

In some embodiments, payloads of the lipoprotein system of the disclosure may include a carbohydrate or a derivative thereof. In some embodiments, the term “carbohydrate” is interchangeably used with saccharide.

The carbohydrate may be a monosaccharide, disaccharide, oligosaccharide, and/or polysaccharide. Non-limiting examples of monosaccharides are glucose, galactose, fructose, and/or xylose. Non-limiting examples of a disaccharide include sucrose, lactose, maltose, trehalose, cellobiose, and/or chitobiose. In some embodiments, the oligosaccharides may be carbohydrates that contains between 3 and 10 single sugar residues, such as trisaccharide, tetra saccharide, or a higher saccharide. Non-limiting examples of polysaccharide include starch, glycogen, galactogen, cellulose, hemicellulose, arabinoxylan, pectin, chitin, dextran, pullulan, chrysolaminarin, curdian, laminarin, lentinan, scleroglucan, gellan, welan, xanthan, agarose, algin, carrageenan, and chitosan.

In some embodiments, the payload may be a carbohydrate. In some embodiments, the carbohydrate derivative may include a carbohydrate molecule is covalently linked to another molecule, e.g., any other polymer molecule.

In some embodiments, the payload may be trehalose. Trehalose is a disaccharide formed by a 1,1-glycosidic bond between two α-glucose units. In a mouse model of tauopathy with Parkinsonism, trehalose consumption improved motor function. Reduced anxiety-related behavior and decreased tau pathology through an apparent increase in autophagic processes (Rodriguez-Navarro et al. Neurobiol Dis 2010; 39(3): 423-38). In vitro treatment utilizing a cellular model for Huntington's disease revealed that trehalose decreased polyglutamine aggregates and/or improved cell survival, with trehalose proving to be the most effective (Tanaka et al. Nat Med 2004; 10(2):148-54).

In some embodiments, the payload may be fucoidan, a long chain sulfated polysaccharide found in various species of brown algae. In an in vitro model of Parkinson's disease, fucoidan from the brown alga Laminaria japonica protected mouse dopaminergic MN9D cells from 1-methyl-4-phenyl-1,2,3,6-tetrahydropyridine (MPTP) toxicity. In an MPTP-exposed mouse model of Parkinson's disease, daily intraperitoneal injections of Laminaria fucoidan reduced locomotor deficits, prevented striatal depletion of dopamine, enhanced brain antioxidant activity, and protected against the loss of dopaminergic neurons in the substantia nigra (Luo et al. Eur J Pharmacol 2009; 617(1-3):33-40; the contents of which are herein incorporated by reference in its entirety). In a rat model of ischemic stroke, rats receiving intravenous infusion of Bladderwrack fucoidan for 7 days following intracerebral hemorrhage induction showed improved motor function and memory retention in a passive avoidance test, despite an increase in hematoma size, compared with the control (Uhm et al. Neurosci Lett 2003; 353(1): 21-4).

In some embodiments, the payload may be a cyclodextrin. Cyclodextrins are a family of cyclic oligosaccharides, composed of glucose units in a ring configuration. They have the shared features of lipophilic central cavities and hydrophilic outer surfaces that help molecules transport lipids in aqueous environments. In an animal study of NPC1 models, Calias demonstrated that, though 2-hydroxypropyl-β-cyclodextrins (HPβCD) does not cross the blood-brain barrier in significant amounts, it can moderately impact CNS manifestations when administered subcutaneously or intraperitoneally to mice at very high doses. Subsequent NPC1 investigations in cats also demonstrated far greater HPβCD efficacy at much lower doses when the drug was administered directly to the CNS (Calias, et al. Current Pharmaceutical Design, 2017, 23, 6231-6238).

### Glycolipids

In some embodiments, a payload of the lipoprotein system disclosed herein may include a glycolipid payload or a glycolipid derivative payload. As used herein, a “glycolipid” refers to a molecule that includes a carbohydrate moiety bound to a lipid moiety which may be represented by general structure G-L, where L is a lipid and G is a carbohydrate. In some embodiments, G is a saccharide, such as, but not limited to hexose or pentose, and may be a mono-, di-, tri-, oligo or polysaccharide, or a derivative thereof. As a non-limiting example, the saccharide may be allose, altrose, glucose, mannose, gulose, idose, galactose, talose, fructose, maltose, lactose and/or sucrose. The linkage between the carbohydrate moiety and the lipid moiety may be at any of the O atoms of the carbohydrate moiety, for example, at position 1, 2, 3 or 4. The linkage may be in the alpha or beta configuration. In some embodiments, the linkage is in the alpha configuration. The payloads of the disclosure may include a structure as described in US Patent, U.S. Pat. No. 9,371,352, the contents of which are incorporated herein by reference in their entirety.

As a non-limiting example, the glycolipid payloads may be a glycoglycerolipid (e.g., galactolipid), a glycosphingolipid (e.g., cerebroside such as galactocerebroside, glucocerebroside, sulfatide, ganglioside, globoside; glycophosphosphingolipid, glycophosphatidylinositol), or a saccharolipid.

In some embodiments, the glycolipid payload may be a glycosphingolipid. Glycosphingolipids are located in the CNS and may be responsible for cell recognition and signaling (Hako Mari et al. J. Biochem. 118, 1091-1103 (1995)). A highly abundant glycosphingolipid in the CNS is ganglioside. Above 90% of the brain ganglioside mass is constituted by four complex gangliosides (GM1, GD1a, GD1b, and GT1b). Gangliosides are enriched in cell membrane microdomains (“lipid rafts”) and play important roles in the modulation of membrane proteins and ion channels, in cell signaling and in the communication among cells. Loss of function mutations in ganglioside biosynthetic enzymes result in severe neurodegenerative disorders, often characterized by very early or childhood onset. Changes in the ganglioside profile have been reported in healthy aging and in common neurological conditions, including Huntington's disease (HD), Alzheimer's disease (AD), Parkinson's disease (PD), amyotrophic lateral sclerosis (ALS), stroke, multiple sclerosis and epilepsy.

In some embodiments, the glycolipid payload may be a galactocerebroside (GalC). A main glycosphingolipid found in myelin is galactocerebroside (GalC). The lipid bilayer of the myelin membrane of the CNS and the peripheral nervous system (PNS) contains the oligodendrocyte- and Schwann cell-specific glycosphingolipids, galactocerebrosides (GalC) and GalC-derived sulfatides (sGalC) respectively. Bosio et al. generated a galactosyltransferase (CGT) null mutant mouse (cgt−/−) with CNS and PNS myelin completely depleted of GalC and derived sGalC. Oligodendrocytes and Schwann cells are unable to restore the structure and function of these galactosphingolipids to maintain the insulator function of the membrane bilayer. They found that the velocity of nerve conduction of homozygous cgt−/− mice is reduced to that of unmyelinated axons and that the severe dysmyelinosis led to death of the cgt−/− mouse, which indicated the essential roles of galactocerebrosides in the maintenance of the normal myelination and thus the healthy nervous systems of mammal animals (Bosio et al. PNAS Nov. 12, 1996, 93 (23) 13280-13285). Together, these data suggest the importance of galactocerebroside in the nervous system.

### Metabolites

In some embodiments, a payload of the lipoprotein system of the disclosure may be a metabolite or a derivative thereof. A metabolite generally refers to any molecule that is an intermediate or product of metabolism or a compound that is necessary for or taking part in a particular metabolic process. A metabolite may not be limited to any particular class of molecules, and includes, for example, classes of compounds such as a lipid, a carbohydrate, an amino acid, an organic acid, and a sterol. A metabolite may be a primary metabolite (i.e., a compound that is directly involved in normal growth, development, and reproduction) or a secondary metabolite (i.e., a compound that is not directly involved in those processes, but usually has an important ecological function). Non-limiting examples of primary metabolites include ethanol, glutamic acid, aspartic acid, 5′ guanylic acid, acetic acid, lactic acid, and/or glycerol. Non-limiting examples of secondary metabolites include pigment, resin, terpenoid (e.g., artemisinin and paclitaxel), tetrapyrrole, ergot, alkaloid, glycoside, antibiotic, glucosinolate, naphthalene, nucleoside, quinolone, peptide, non-ribosomal peptide, phenazine, phenol, and/or polyketide. In some embodiments, a metabolite may be a low molecular weight compound (<1 kDa) that is the product of a chemical reaction or reactions that occur inside cells of living organisms, such as within human or animal or plant tissue. Living organisms also include microorganisms, such as bacteria and fungus. In some embodiments, a metabolite is an intermediate or a product of an enzymatic reaction, such as an enzymatic reaction that occurs within the cells of normal tissue or abnormal tissue, such as diseased tissue, such as cancerous tissue.

### Small Molecules

In some embodiments, a payload of the lipoprotein system of the disclosure may be a small molecule. The term “small molecule,” as used herein, generally refers to an organic molecule that is less than 2000 g/mol in molecular weight, less than 1500 g/mol, less than 1000 g/mol, less than 800 g/mol, or less than 500 g/mol. Small molecules may be non-polymeric and/or non-oligomeric. The compound may be a small molecule having a molecular weight of less than about 2,000 Da, 1,500 Da 1,000 Da, less than about 900 Da, less than about 800 Da, less than about 700 Da, less than about 600 Da or less than about 500 Da. In some embodiments, the compound may have a molecular weight of between about 5 Da and about 1,000 Da, between about 10 Da and about 900 Da, in some embodiments between about 20 Da and about 700 Da, in some embodiments about 20 Da and about 500 Da, between about 50 Da and about 400 Da, in some embodiments between about 100 Da and about 300 Da, and in some embodiments between about 150 Da and about 300 Da. The molecular weight of the compound may be calculated as the sum of the atomic weight of each atom in the formula of the compound multiplied by the number of each atom. It may also be measured by mass spectrometry, NMR, chromatography, light scattering, viscosity, and/or any other methods known in the art. It is known in the art that the unit of molecular weight may be g/mol, Dalton (Da), or atomic mass unit (amu), wherein 1 g/mol=1 Da=1 amu.

In some embodiments, the small molecule may include one or more functional groups. Non-limiting examples of functional groups include H, halogen, CF₃, —OH, —O(C1-8 alkyl), —NH₂, —SH, —SC₁₋₈ alkyl, —CN, —NO₂, —CH₂(NH₂), —C(O)OH, —S(O)₂NH₂, —C(O)NH₂, —C(O)C1-8 alkyl, NHC(O)—C₁₋₈ alkyl, N(C₁-alkyl)C(O)—C₁₋₈ alkyl, OC(O)NH₂, OC(O)NH(CH₃), OC(O)N(CH₃)₂, optionally substituted C₁₋₈ alkyl, optionally substituted C₂₋₈ alkenyl, optionally substituted C₂₋₈ alkynyl, optionally substituted C₃₋₈ cycloalkyl, optionally substituted 5-10 membered heterocyclic, optionally substituted aryl, optionally substituted 5-10 membered heteroaryl or optionally substituted acyl, wherein the optional substituents for alkyl, alkenyl, alkynyl, aryl, heteroaryl, cyclic, heterocyclic and acyl are one or more substituents independently selected from the group consisting of halogen, OH, NH₂, CH₃, CF₃, —CN, —NO₂, —C(O)OH, —S(O)₂NH₂, —C(O)NH₂, —CH₂NH₂, —C(O)CH₃, SH, —S—CH₃, optionally substituted C₁₋₆ alkyl, and optionally substituted C₃₋₆ cycloalkyl, and wherein the C₁₋₆ alkyl and C₃₋₈ cycloalkyl optional substituents are one or more substituents independently selected from the group consisting of halogen, OH, NH₂, CH₃, CF₃, —CN, —NO₂, —C(O)OH, —S(O)₂NH₂, —C(O)NH₂, —CH₂NH₂, —C(O)CH₃, SH, and —S—CH₃.

In some embodiments, the small molecule may be cell permeable. The small molecule may be a therapeutic agent, or a diagnostic agent.

### Therapeutic Agents

In some embodiments, the small molecule may be a therapeutic agent such as an antineoplastic agent, an analgesic, an anesthetic, an antibacterial, an anticonvulsant, anti-cancer agent, an antidementia agent, an antidepressant, an antiemetic, an antifungal, an antigout agent, an anti-inflammatory agent, an anti-infective agent, an antimigraine agent, an antimyasthenic agent, an antimycobacterial agent, an antiparasitic, an antiparkinson agent, an antipsychotic, an antispasticity agent, an antiviral, an anxiolytic, a bipolar agent, a blood glucose regulator, a blood product, a blood modifier, a blood volume expander, a chemotherapeutic agent, a cardiovascular agent, a cytotoxin, a central nervous system agent, a dental and oral agent, a dermatological agent, an electrolyte, an enzyme replacement, an enzyme modifier, a gastrointestinal agent, a genitourinary agent, an immunological agent, an immunoinhibitory agent, an immunostimulatory agent, an inflammatory bowel disease agent, a metabolic bone disease agent, an ophthalmic agent, an otic agent, a respiratory tract agent, a sedative, a hypnotic, a skeletal muscle relaxant, a caspase inhibitor, a radioactive ion, and/or a bioactive molecule.

In some embodiments, the therapeutic agent is a cPLA2 inhibitor. As a non-limiting example, the cPLA2 inhibitor may be able to curb neuroinflammation and/or improve brain uptake of fatty acids.

The therapeutic agent may be an agent for treating a disease such as Parkinson's Disease (PD), Dementia with Lewy Bodies (DLB), Multiple System Atrophy (MSA), Decreased muscle mass, Spinal muscular atrophy (SMA) Alzheimer's disease (AD), Amyotrophic lateral sclerosis (ALS), Huntington's Disease (HD), Multiple sclerosis (MS), Stroke, Migraine, Pain, Neuropathies, Psychiatric disorders including schizophrenia, bipolar disorder, and autism, Cancer, ocular disease, systemic diseases of the blood, heart and bone, Immune system and Autoimmune disease, or Inflammatory disease.

In some embodiments, the therapeutic agent may be an antioxidant. Non-limiting examples of the antioxidant are alpha-tocopherol, ascorbic acid, citric acid, coenzyme Q (e.g., CoQ6, CoQ10), malic acid, methionine, uric acid, caffeic acid, monothioglycerol, phosphoric acid, and potassium metabisulfite. In some embodiments, the therapeutic agent is an antioxidant, and the antioxidant is alpha-tocopherol.

In some embodiments, the therapeutic agent may include an immunoinhibitory agent. As a non-limiting example, the immunoinhibitory agent includes a retinoic acid, dexamethasone, or cyclophosphamide.

In some embodiments, the therapeutic agent may be an antibiotic (e.g., cephalospori, gentamicin, flucloxacillin, and vancomycin), a statin (e.g., atorvastatin, fluvastatin, lovastatin, pitavastatin, pravastatin, simvastatin, and rosuvastatin), or an immunogen (e.g., haptens).

In some embodiments, the therapeutic agent may be a caspase inhibitor such as a caspase-1 inhibitor, a caspase-3 inhibitor, or a caspase-8 inhibitor. Caspase inhibitors may be useful for treating non-alcoholic fatty liver disease, epilepsy, ischemic disorders, Huntington's disease, amyotrophic lateral sclerosis (ALS), autoimmune diseases such as rheumatoid arthritis, osteoarthritis, inflammatory bowel disease, viral infections (e.g., hepatitis C) and sepsis. Non-limiting examples of caspase inhibitors that can be included in the lipoprotein system include emricasan, pralnacasan, VX-765, and other caspase inhibitors described in US Patent Publication US 20100041661, International Publication WO2001021600, and U.S. Pat. No. 9,365,612.

In some embodiments, the therapeutic agent may be an agonist of Liver-X-Receptor (LXR) and/or an agonist of retinoid X receptor (RXR). In some embodiments, the LXR and/or RXR agonist may be hypocholamide, T0901317, GW3965, 22(R)-hydroxycholesterol, 24(S)-hydroxycholesterol, N,N-dimethyl-3beta-hydroxy-cholenamide (DMHCA), Bexarotene, 3-[4-Hydroxy-3-(5,6,7,8-tetrahydro-3,5,5,8,8-pentamethyl-2-naphthalenyl)phenyl]-2-propenoic acid (CD 3254), Docosahexaenoic acid, 6-[1-(5,6,7,8-Tetrahydro-3,5,5,8,8-pentamethyl-2-naphthalenyl)cyclopropyl]-3-pyridinecarboxylic acid (LG 100268), 4-[2-(5,6,7,8-Tetrahydro-5,5,8,8-tetramethyl-2-naphthalenyl)-1,3-dioxolan-2-yl]-benzoic acid (SR11237), 3-[3-[[[2-Chloro-3-(trifluoromethyl)phenyl]methyl](2,2-diphenylethyl)amino]propoxy]benzeneacetic acid hydrochloride (GW 3965), 2-[(2-Chloro-4-fluorophenyl)methyl]-3-(4-fluorophenyl)-7-(trifluoromethyl)-2H-indazole (WAY 252623), and/or 27-Hydroxycholesterol.

In some embodiments, the therapeutic agent may be the Peroxisome Proliferator Activated Receptor Gamma (PPARγ) agonist pioglitazone. In some embodiments, the therapeutic agent may be the Acyl-CoA:cholesterol acyltransferase 1 (ACAT1) enzyme inhibitor.

In some embodiments, the therapeutic agent may be a cytotoxin or cytotoxic agent including any agent that may be detrimental to cells. Examples include, but are not limited to, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020 incorporated herein in its entirety), rachelmycin (CC-1065, see U.S. Pat. Nos. 5,475,092, 5,585,499, and 5,846,545, all of which are incorporated herein by reference), and analogs or homologs thereof.

In some embodiments, the therapeutic agent may be a radioactive ion including, without limitation, iodine (e.g., iodine 125 or iodine 131), strontium 89, phosphorous, palladium, cesium, iridium, phosphate, cobalt, yttrium 90, samarium 153, and praseodymium.

In some embodiments, the therapeutic agent may be a bioactive agent, which is optionally deuterated. Highly deuterated bioactive agents can be useful for inhibiting biological processes. Non-limiting examples of bioactive agent include polyphenols, such as flavonoids (e.g., anthoxanthins such as luteolin, apigenin, tangeritin, quercetin, kaempferol, myricetin, fisetin, galangin, isorhamnetin, pachypodol, rhamnazin, pyranoflavonols, and furanoflavonols; flavanones such as hesperetin, naringenin, eriodictyol, and homoeriodictyol; flavanonols such as dihydroquercetin and dihydrokaempferol; flavans such as catechin, gallocatechin, catechin 3-gallate, gallocatechin 3-gallate, epicatechins, epigallocatechin, epicatechin 3-gallate, epigallocatechin 3-gallate, theaflavin, and leucoanthocyanidin), carotenoids (e.g., beta-carotene, alpha-carotene, beta-cryptoxanthin and gamma-carotene, lutein, lycopene, astaxanthin, zeaxanthin) and phytosterols.

In some embodiments, the therapeutic agent may be an antimetabolite (e.g., methotrexate, 6-mercaptopurine, 6-thioguanine, cytarabine, 5-fluorouracil decarbazine), alkylating agents (e.g., mechlorethamine, thiotepa chlorambucil, rachelmycin (CC-1065), melphalan, carmustine (BSNU), lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In some embodiments, the compositions as described herein may include a therapeutic agent. In some embodiments, the therapeutic agent may be noncovalently sequestered in the compositions as described herein. In some embodiments, the therapeutic agent may be covalently linked to the apolipoproteins of the disclosure. In some embodiments, the therapeutic agent may be covalently linked to the head group of the lipid. In some embodiments, the therapeutic agent may be otherwise associated with the lipoprotein systems as described herein. In some embodiments, the therapeutic agent is loaded into the lipoprotein systems as described herein. In some embodiments, the therapeutic agent is encapsulated or embedded within the lipoprotein systems as described herein by “passive” loading techniques that involve the incorporation of therapeutic agents into the lipoprotein systems as described herein during the lipoprotein systems self-assembly process in solution. Alternatively, in some embodiments, the therapeutic agents may be actively loaded into the lipoprotein systems as described herein. For example, in some embodiments, the lipoprotein systems may be exposed to conditions, such as electroporation, in which the bilayer membrane is made permeable to a solution containing therapeutic agent thereby allowing for the therapeutic agents to enter into the lipoprotein systems. Loading of the lipoprotein systems with therapeutic agents or other components may be carried out via other methods known in the art, as disclosed for example in the following references: de Villiers, M. M. et al., Eds., Nanotechnology in Drug Delivery, Springer (2009); Gregoriadis, G., Ed., Liposome Technology: Entrapment of drugs and other materials into liposomes, CRC Press (2006) (the contents of each of which are herein incorporated by reference in their entirety).

### Diagnostic Agents

In some embodiments, the small molecule payloads may include a diagnostic agent. As a non-limiting example, the diagnostic agent may be an agent that is detectable (herein a detectable agent) such as organic small molecules, inorganic compounds, nanoparticles, enzymes or enzyme substrates, fluorescent materials, luminescent materials (e.g., luminol), bioluminescent materials (e.g., luciferase, luciferin, and aequorin), chemiluminescent materials, dyes and stains, radioactive materials (e.g., ¹⁸F, ⁶⁷Ga, ^{81m}Kr, ⁸²Rb, ¹¹¹In, ¹²³I, ¹³³Xe, ²⁰¹TI, ¹²⁵I, ³⁵S, ¹⁴C, ³H, or ^{99m}Tc (e.g., as pertechnetate (technetate(VII), TcO₄⁻)), and contrast agents (e.g., gold (e.g., gold nanoparticles), gadolinium (e.g., chelated Gd), iron oxides (e.g., superparamagnetic iron oxide (SPIO), monocrystalline iron oxide nanoparticles (MIONs), and ultrasmall superparamagnetic iron oxide (USPIO)), and manganese chelates (e.g., Mn-DPDP), barium sulfate, iodinated contrast media (iohexol), microbubbles, or perfluorocarbons). More examples of diagnostic agents are described in Patel et al., Orient. J. Chem., Vol. 28(2), 999-1004 (2012), of which the contents are incorporated by reference in its entirety.

In some embodiments, the lipoprotein systems as described herein may also include additional components such as diagnostic agents. In some embodiments, the diagnostic agents may be associated with the lipoprotein systems as described herein in a variety of ways including, for example, being covalently bound to a lipoprotein system component or noncovalently embedded or encapsulated in the lipoprotein systems as described herein. A diagnostic agent may include any diagnostic agent known in the art, as provided, for example, in the following references: Armstrong et al., Diagnostic Imaging, 5th Ed., Blackwell Publishing (2004); Torchilin, V. P., Ed., Targeted Delivery of Imaging Agents, CRC Press (1995); Vallabhajosula, S., Molecular Imaging: Radiopharmaceuticals for PET and SPECT, Springer (2009).

In some embodiments, a diagnostic agent may include chelators that bind to metal ions to be used for a variety of diagnostic imaging techniques. In some embodiments, a radioisotope may be incorporated into some of the diagnostic agents and may include radionuclides that emit gamma rays, positrons, beta and alpha particles, and X-rays. In some embodiments, the diagnostic agents may include optical agents, such as fluorescent agents, phosphorescent agents, chemiluminescent agents, and the like. Numerous agents (e.g., dyes, probes, labels, or indicators) are known in the art and may be used in the present disclosure. (See, e.g., Invitrogen, The Handbook—A Guide to Fluorescent Probes and Labeling Technologies, Tenth Edition (2005)). In some embodiments, the diagnostic agents may include magnetic resonance (MR) and x-ray contrast agents that are known in the art, including, for example, iodine-based x-ray contrast agents, superparamagnetic iron oxide (SPIO), complexes of gadolinium or manganese, and the like. (See, e.g., Armstrong et al., Diagnostic Imaging, 5th Ed., Blackwell Publishing (2004)).

### Lipoprotein System Modifier

Lipoprotein systems of the disclosure may include one or more lipoprotein system modifiers. As used herein, the term “lipoprotein system modifier” refers to any macro molecule or micro molecule that can alter/tune the location, structure, function, solubility, detection, synthesis, assembly, and/or degradation of a lipoprotein system in vitro, ex vivo or in vivo.

The lipoprotein system modifier may be, e.g., a targeting agent, a regulatory agent, a solubilizing agent, a stabilizing agent, or a detection agent.

In some embodiments, the lipoprotein systems of the disclosure may include modifiers e.g., paraoxonase (PON), antioxidants, cyclodextrins, or other materials that retain a lipid of the lipoprotein systems.

In some embodiments, the lipoprotein systems of the disclosure may include a surfactant, located at the lipid-water interface, to reduce the interfacial tension between a lipid and the aqueous phase.

Lipoprotein system modifiers may be used to tune or alter detection of the compositions of the disclosure by the immune system. In some embodiments, the lipoprotein systems of the disclosure may be coated with biocompatible inert polymers to render them invisible to the cell, tissues, or systems of the organism. For example, lipoprotein systems of the disclosure may be coated with poly-ethylene glycol (PEG).

### Regulatory Agent

In some embodiments, the lipoprotein system modifier may be a regulatory agent. For example, the modifier may be an agent such as a peptide, protein or nucleic acid that can alter the binding of the apolipoprotein systems of the disclosure to their cognate receptors.

### Solubilizing Agent

In some embodiments, the lipoprotein system modifier may be a solubilizing agent which may be used to alter the solubility of the systems of the disclosure in a hydrophilic phase, a hydrophobic phase, aqueous phase or organic phase.

### Targeting Agent

In some embodiments, the lipoprotein system modifier may be a targeting agent. As used herein, the term “targeting agent” refers to a functional group or moiety attached to an agent that facilitates localization of the lipoprotein system or a component thereof to a desired region, tissue, cell and/or protein. Such targeting agents may include, but are not limited to, cell or tissue targeting agents or groups (e.g., lectins, glycoproteins, lipids, proteins, an antibody that binds to a specified cell type such as a kidney cell or other cell type). In one embodiment, the targeting agent may be a lipid such as, but not limited to, phosphatidyl serine. In some embodiments, targeting agents may include thyrotropins, melanotropins, lectins, glycoproteins, surfactant protein A, mucin carbohydrates, multivalent lactose, multivalent galactose, N-acetyl-galactosamine, N-acetyl-gulucosamine, multivalent mannose, multivalent fucose, glycosylated polyaminoacids, multivalent galactose, transferrin, bisphosphonate, polyglutamate, polyaspartate, lipids, cholesterol, steroids, bile acids, folates, vitamin B12, biotin, an RGD peptide, an RGD peptide mimetic or an aptamer. In some embodiments, targeting agents may be proteins, e.g., glycoproteins, or peptides, e.g., molecules having a specific affinity for a co-ligand, or antibodies e.g., an antibody, which binds to a specified cell type such as a cancer cell, endothelial cell, or bone cell. Targeting agents may also include hormones and/or hormone receptors. In some embodiments, targeting agent may be any ligand capable of targeting specific receptors. Examples include, without limitation, folate, GalNAc, galactose, mannose, mannose-6-phosphate, apatamers, integrin receptor ligands, chemokine receptor ligands, transferrin, biotin, serotonin receptor ligands, PSMA, endothelin, GCPII, somatostatin, LDL, and HDL ligands. In some embodiments, targeting groups are aptamers. Such aptamers may be unmodified or include any combination of modifications disclosed herein.

In some embodiments, targeted biodistribution of lipoprotein systems may be improved by coating with antibodies to deliver the lipoprotein systems to particular cell or tissue types.

In some embodiments, the lipoprotein systems as described herein may also include one or more targeting agents. Generally, a targeting agent associates with any target of interest, such as a target associated with an organ, tissues, cell, extracellular matrix, or intracellular region. In some embodiments, a target may be associated with a particular disease state. In some embodiments, the targeting component may be specific to only one target, such as a receptor. Suitable targets may include, but are not limited to, a nucleic acid such as a DNA, RNA and modified derivatives thereof, a protein such as an extracellular protein, a receptor, a cell surface receptor, a transmembrane protein, an enzyme and an antibody, and a carbohydrate such as a monosaccharide, disaccharide and polysaccharide that may be, for example, present on the surface of a cell. In some embodiments, the targeting agent may be a target ligand, a small molecule mimic of a target ligand (e.g., a peptide mimetic ligand), an antibody or antibody fragment specific for a particular target, or an aptamer. In some embodiments, the targeting agents may be associated with the lipoprotein systems as described herein in a variety of ways including, for example, being covalently bound to a lipoprotein system component or noncovalently embedded or encapsulated in the lipoprotein system.

### Stabilizing Agent

In some embodiments, the lipoprotein system modifier may be an agent that reduces or prevents the physical or chemical degradation of the system. For example, the stabilizing agent may be a cryoprotectant, a chelator, an antioxidant, or any combination thereof. In some embodiments, the stabilizing agent may be a carbohydrate such as, but not limited to, mannitol and/or xylitol.

In some embodiments, the lipoprotein system modifiers may include antioxidants to prevent the oxidation and/or decomposition of a lipid of the disclosure. The antioxidant may be selected from, but not limited to, alpha-tocopherol, ascorbic acid, citric acid, a coenzyme Q (e.g., CoQ6 and/or CoQ10), malic acid, methionine, uric acid, caffeic acid, monothioglycerol, phosphoric acid, and potassium metabisulfite.

### Penetrating Agent

In some embodiments, the penetrating agent may be a cell penetrating agent which may facilitate cellular update of molecules. In some embodiments, the cell-penetrating polypeptide may include a first domain and a second domain. The first domain may include a supercharged polypeptide. The second domain may include a protein-binding partner. In a further embodiment, the cell-penetrating polypeptide is capable of penetrating a second cell which may be present in the same microenvironment, tissues or organ as the first cell.

In some embodiments, the lipoprotein systems of the disclosure may include a penetrating agent that permits penetration through the blood-brain barrier, vascular barrier, or other epithelial barriers. The penetrating agent may be a blood-brain barrier penetrating agent. Blood-brain barrier penetrating agents include, but are not limited to, glucose transporter type 1 (GLUT1), cationic amino-acid transporter type 1 (CAT1), monocarboxylic acid transporter type 1 (MCT1), concentrative nucleoside transporter type 2 (CNT2), active efflux transporter (AET) (e.g., p-glycoprotein, or any agents described in Pardridge, J et al. (Cereb Blood Flow Metab 32(11): 1959-1972, 2012; the contents of which are herein incorporated by reference in their entirety), Additional blood-brain barrier penetrating moieties are known in the art. Agents that cross the brain blood barrier may be used. For example, some cell penetrating peptides that can target molecules to the brain blood barrier endothelium may be used for formulation (e.g., Mathupala, Expert Opin Ther Pat., 2009, 19, 137-140; the content of which is incorporated herein by reference in its entirety).

In some embodiments, the payloads in the disclosure may contain BBB-transport proteins or polypeptides, which can facilitate the delivery of the lipoprotein system across the blood-brain barrier (BBB) and into organs, tissues, or cells of the central nervous system (CNS).

In some embodiments, BBB penetrating agents can improve pharmacokinetics of the lipoprotein system across the blood-brain barrier (BBB) and into organs, tissues, or cells of the central nervous system (CNS).

Non-limiting examples of the BBB penetrating agents include an aprotinin peptide, a glutathione (GSH), a human low-density lipoprotein receptor (hLDLR) binding peptide, a p97 (melanotransferrin) polypeptide, a Receptor Associated Protein (RAP), protein transduction domain (PTD), an antibody or natural ligand that binds to a BBB-associated receptor, and any analog of the above.

### Detection Agent

In some embodiments, the lipoprotein system modifier may be a detection agent, which may allow tracking the systems of the disclosure in a cell, tissue or an organism. Such detection agents may include but are not limited to biotin labels, ubiquitins, fluorescent molecules, human influenza hemagglutinin (HA), c-myc, histidine (His), flag, glutathione S-transferase (GST), V5 (a paramyxovirus of simian virus 5 epitope), biotin, avidin, streptavidin, horse radish peroxidase (HRP), azido functionalization, alkynyl functionalization, and digoxigenin.

In some embodiments, the detection agent may be a dye, e.g., a fluorophore such as an amphiphilic molecule having a charged fluorophore group. In certain embodiments, the dye may be a dialkylcarbocyanine probe such as Dil, DiO, DiD, DiR, or an analog thereof. In other embodiments, the detection agent may be an amphiphilic or nonpolar dye. Dyes include, for example, and without limitation, amphiphilic derivative of rhodamine, fluorescein, or coumarin such as octadecyl rhodamine B, 5-dodecanoyl-amino fluorescein, 5-hexadecanoyl-fluorescein, 5-octadecanolyl-amino fluorescein, and 4-heptadecyl-7-hydroxy coumarin. Diphenylhexatriene (DPH), Trimethylammonium DPH, Trimethylammonium phosphate DPH, DPH propionic acid, or a nonpolar BODIPY fluorophore. In some embodiments, the dye is a lipid-partitioning fluorescent molecule. In others, the dye may be a nonpolar pyrenes, Nile red, bimane azide, prodan, laurdan, dapoxyl derivatives, anilinonaphthalenesulfonate (ANS), bis ANS, DCVJ, or 4-amino-4′-benzamidostilbene-2,2′-disulfonic acid.

In some embodiments, the detection agent may be a fluorescent protein such as GFP, EGFP, BFP, CFP, RFP, or YFP or fluorescent variants thereof with at least 80% sequence identity thereto.

The detection agent may be a fluorescent label which can be incorporated into the lipoprotein systems of the disclosure. For example, classes of lipophilic dyes that associate with lipids are provided in the Molecular Probes Handbook, 10th edition (Chapter 13). For example, fatty acids labeled with BODIPY fluorophores (BODIPY 503/512, BODIPY 500/510, BODIPY 530/550, BODIPY 558/568, BODIPY 581/591), nitrobenzodiazole (NBD), and pyrene, as well as dansyl undecanoic acid (DAUSA) and cis-parinaric acid are available from Molecular Probes (Eugene Oregon). Phospoholipids can also be labeled with BODIPY dyes; for example, BODIPY FL dye-labeled phosphtidic acid, BODIPY 530/550-labeled glycerophophocholine, and BODIPY 581/591-labeled glycerophosphocholine are all commercially available. The phospholipid analog beta-DPH HPC and derivatives as well as phospholipids with NBD-labeled acyl chains and purene-labeled acyl chains can also be incorporated into systems of the disclosure.

### Polynucleotides

Lipoprotein systems components including, but not limited to, polypeptides and payloads may be encoded by nucleic acids. Payloads of the disclosure may also be nucleic acids or nucleic acid-based. Polynucleotides of the disclosure include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

In some embodiments, the polynucleotides of the disclosure may be or may be derived from or include a portion of a pseudogene. As used herein, a pseudogene refers to a nucleic acid sequence that resembles a gene but has been mutated over the course of evolution such that it may not translate into a polypeptide sequence. Pseudogenes may lack introns and multiple stop codons may be present in the sequence. Though genetically similar to the original functional gene, pseudogenes do not result in functional proteins. Pseudogenes may be transcriptionally active, but also capable of influencing the activity of other genes and affecting the phenotype of the organism. Pseudogenes interfere with the expression of other genes by several well-defined mechanisms. Pseudogenes can encode for an antisense RNA that inhibits the translation of the parent gene or generate a small interfering RNA that leads to gene silencing. Pseudogenes can also compete for regulatory factors, including transcription factors (via its promoter or other regulatory sites) and microRNA (via the 3′-untranslated region of the noncoding transcript).

In some embodiments, the polynucleotide may be a messenger RNA (mRNA). As used herein, the term “messenger RNA” (mRNA) refers to any polynucleotide which encodes a polypeptide of interest and which is capable of being translated to produce the encoded polypeptide of interest in vitro, in vivo, in situ or ex vivo. Polynucleotides of the disclosure may be mRNA or any nucleic acid molecule and may or may not be chemically modified. In some embodiments, polynucleotides of the disclosure may be a DNA sequence that is identical to an mRNA sequence and herein referred to as cDNA.

Traditionally, the basic components of an mRNA molecule include at least a coding region, a 5′-UTR, a 3′-UTR, a 5′ cap, and a poly-A tail. Building on this wild type modular structure, the present disclosure expands the scope of functionality of traditional mRNA molecules by providing payloads constructs which maintain a modular organization, but which include one or more structural and/or chemical modifications or alterations which impart useful properties to the polynucleotide, for example tenability of function. As used herein, a “structural” feature or modification is one in which two or more linked nucleosides are inserted, deleted, duplicated, inverted or randomized in a polynucleotide without significant chemical modification to the nucleosides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In some embodiments, polynucleotides of the present disclosure may harbor 5′UTR sequences which play a role in translation initiation. 5′UTR sequences may include features such as Kozak sequences which are commonly known to be involved in the process by which the ribosome initiates translation of genes, Kozak sequences have the consensus XCCR(A/G) CCAUG, where R is a purine (adenine or guanine) three bases upstream of the start codon (AUG) and X is any nucleotide. In one embodiment, the Kozak sequence is ACCGCC. By engineering the features that are typically found in abundantly expressed genes of target cells or tissues, the stability and protein production of the polynucleotides of the disclosure can be enhanced.

Further provided are polynucleotides, which may contain an internal ribosome entry site (IRES) which play an important role in initiating protein synthesis in the absence of 5′ cap structure in the polynucleotide. An IRES may act as the sole ribosome binding site or may serve as one of the multiple binding sites. Polynucleotides of the disclosure containing more than one functional ribosome binding site may encode several peptides or polypeptides that are translated independently by the ribosomes giving rise to bicistronic and/or multicistronic nucleic acid molecules.

In some embodiments, polynucleotides of the disclosure which may encode a lipoprotein system component, a polypeptide or a payloads may include from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000 nucleotides).

Other regions of the polynucleotides which encode certain features such as for example cleavage sites, trafficking signals such as localization signals or smaller features may range independently from 15-1,000 nucleotides in length (e.g., greater than 30, 40, 45, 50, 55, 60, 70, 80, 90, 100, 120, 140, 160, 180, 200, 250, 300, 350, 400, 450, 500, 600, 700, 800, and 900 nucleotides or at least 30, 40, 45, 50, 55, 60, 70, 80, 90, 100, 120, 140, 160, 180, 200, 250, 300, 350, 400, 450, 500, 600, 700, 800, 900, and 1,000 nucleotides).

According to the present disclosure, multiple distinct polypeptides or payloads may be linked together through the 3′-end using nucleotides which are modified at the 3′-terminus. For payloads, chemical conjugation may be used to control the stoichiometry of delivery into cells.

The polynucleotides of the disclosure, their regions or parts or subregions may be codon optimized. Codon optimization methods are known in the art and may be useful in efforts to achieve one or more of several goals. These goals include to match codon frequencies in target and host organisms to ensure proper folding, bias nucleotide content to alter stability or reduce secondary structures, minimize tandem repeat codons or base runs that may impair gene construction or expression, customize transcriptional and translational control regions, insert or remove protein signaling sequences, remove/add post translation modification sites in encoded protein (e.g., glycosylation sites), add, remove or shuffle protein domains, insert or delete restriction sites, modify ribosome binding sites and degradation sites, to adjust translational rates to allow the various domains of the protein to fold properly, or to reduce or eliminate problem secondary structures within the polynucleotide. Codon optimization tools, algorithms and services are known in the art, and non-limiting examples include services from GeneArt (Life Technologies), DNA2.0 (Menlo Park CA), OptimumGene (GenScript, Piscataway, NJ), algorithms such as but not limited to, DNAWorks v3.2.3 and/or proprietary methods. In one embodiment, a polynucleotide sequence or portion thereof is codon optimized using optimization algorithms. Codon options for each amino acid are well-known in the art as are various species table for optimizing for expression in that particular species.

In some embodiments of the disclosure, certain polynucleotide features may be codon optimized. For example, a preferred region for codon optimization may be upstream (5′) or downstream (3′) to a region which encodes a polypeptide. These regions may be incorporated into the polynucleotide before and/or after codon optimization of the payloads encoding region or open reading frame (ORF).

After optimization (if desired), the polynucleotides components are reconstituted and transformed into a vector such as, but not limited to, plasmids, viruses, cosmids, and artificial chromosomes.

In some embodiments, the polynucleotides of the disclosure may include elements to enhance the specificity and expression of the polynucleotides described herein, including CMV enhancers, upstream enhancers (USEs), introns, endogenous miRNAs, post-transcriptional regulatory elements (PREs), polyadenylation (PolyA) signal sequences, and promoters.

In some embodiments, polynucleotides may include promoters which drive or promote expression in most tissues include, but are not limited to, human elongation factor 1α-subunit (EF1α), cytomegalovirus (CMV) immediate-early enhancer and/or promoter, chicken β-actin (CBA; such as, but not limited to, a CBA promoter as described in Miyazaki et al. (Gene. 1989 Jul. 15; 79(2):269-77, the contents of which are herein incorporated by reference in its entirety)) and its derivative CAG, p glucuronidase (GUSB), or ubiquitin C (UBC).

Tissue-specific expression elements may be used to restrict expression of the polynucleotides to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

Non-limiting examples of tissue-specific expression elements for neurons include neuron-specific enolase (NSE), platelet-derived growth factor (PDGF), platelet-derived growth factor B-chain (PDGF-p), synapsin (Syn or Syn1), methyl-CpG binding protein 2 (MeCP2), Ca2+/calmodulin-dependent protein kinase II (CaMKII), metabotropic glutamate receptor 2 (mGluR2), neurofilament light (NFL) or heavy (NFH) chain, β-globin minigene n32, preproenkephalin (PPE), enkephalin (Enk), VGF, and excitatory amino acid transporter 2 (EAAT2) promoters. A non-limiting example of tissue-specific expression elements for neuroectodermal stem cells is nestin.

Non-limiting examples of tissue-specific expression elements for astrocytes are glial fibrillary acidic protein (GFAP, GFabc1D) and EAAT2 promoters. A non-limiting example of a tissue-specific expression element for oligodendrocytes includes the myelin basic protein (MBP) promoter. Non-limiting examples of muscle-specific promoters include Mb promoter, myosin promoter, dystrophin promoter, dMCK, tMCK mammalian muscle creatine kinase (MCK) promoter, mammalian desmin (DES) promoter, mammalian troponin I (TNNI2) promoter, and mammalian skeletal alpha-actin (ASKA) promoter (see, e.g., U.S. Patent Publication US20110212529, the contents of which are herein incorporated by reference in their entirety).

Non-limiting examples of blood-specific promoters include B29 promoter, immunoglobulin heavy chain promoter, CD45 promoter, mouse INF-β promoter, CD45 SV40/CD45 promoter, WASP promoter, CD43 promoter, CD43 SV40/CD43 promoter, CD68 promoter, GPIIb promoter, CD14 promoter, and CD2 promoter. Non-limiting examples of bone-specific promoters include osteocalcin, bone sialoprotein, and OG-2 promoter. Non-limiting examples of ocular promoters include Chx10, PrP, Dkk3, Math5, Ptf1a, Pcp2, Nefh, gamma-synuclein gene (SNCG), Grik4, Pdgfra, Chat, Thy1.2, hVmd2, Thy1, Modified αA-crystallin, hRgp, mMo, Opn4, RLBP1, Glast, Foxg1, hVmd2, Trp1, Six3, cx36, Grm6-SV40 eukaryotic promoter, hVmd2, Dct, Rpc65, mRho, Irbp, hRho, Pcp2, Rhodopsin, and mSo. Non-limiting examples of cardiac promoters include MLC2v promoter, αMHC promoter, rat troponin T (Tnnt2), Tie2, and Tcf21. Non-limiting examples of kidney-specific promoters include ECAD, NKCC2, KSPC, NPHS1, and SGLT2. Non-limiting examples of liver-specific promoters include SV40/bA1b promoter, SV40/hA1b promoter, Hepatitis B virus core promoter, and Alpha fetoprotein. Non-limiting examples of lung-specific promoters include Surfactant protein B promoter and Surfactant protein C promoter. Non-limiting examples of pancreas-specific promoters include elastase-1 promoter, PDX1 promoter, and insulin promoter. Non-limiting examples of vascular- or vasculature-specific promoters include Slco1c1, tie, cadherin, ICAM-2, claudin 1, Cldn5, Flt-1 promoter, and Endoglin promoter.

### Preparation of Lipoprotein Systems

### Polypeptide Synthesis and Storage

Provided here are methods of making the polypeptides of the present disclosure. The polypeptides as described herein can be generated by any methods known in the art. In some embodiments, the polypeptides as described herein are generated by introducing the polynucleotides encoding the polypeptides into cells, expressing and purifying the polypeptides as described herein.

In some embodiments, the polypeptides as described herein are generated by (a): transfecting cells with the plasmids comprising the polynucleotides encoding the polypeptides as described herein, expressing and purifying the polypeptides as described herein, and (b): performing post-translational modifications (PTMs) on the polypeptides as described herein. In some embodiments, the polypeptides of the disclosure may be subject to post translational modifications after the lipoprotein system assembly.

Post-translational modifications (PTMs) of the present disclosure include, but are not limited to oxidation, carbamylation, oxi-carbamylation, acetylation, phosphorylation, ubiqutination, biotinylation, carboxylation, deamidation, deamination, deacetylation, dihydroxylation, dephosphorylation, formylation, gamma-carboxyglutamation, glutathionylation, glycation, hydroxylation, methylation, nitration, sumoylation, N- or O-transglutamination, N- or O-glycosylation and farnesylation. Polypeptides may include 1, 2, 3, 4, 5, 6, 7, 8, 9, 10 or more post-translational modifications. In some embodiments, PTMs may be present in the structured sequence region, N terminal sequence region, C terminal sequence region or a combination thereof. In some embodiments, PTMs may be performed on the polypeptides before lipoprotein system assembly. In some embodiments, PTMs may be performed on the polypeptides after lipoprotein system assembly. In some embodiments, the polypeptides as described herein are stored in solutions or buffers containing 0.01-100 M, 0.05-100 M, 0.1-100 M, 0.5-100 M, 1-100 M, 2-100 M, 3-100 M, 4-100 M, 5-100 M, 6-100 M, 7-100 M, 8-100 M, 9-100 M, 10-100 M, 15-100 M, 20-100 M, 25-100 M, 30-100 M, 35-100 M, 40-100 M, 45-100 M, 50-100 M, 55-100 M, 60-100 M, 65-100 M, 70-100 M, 75-100 M, 80-100 M, 85-100 M, 90-100 M, or 95-100 M guanidinium hydrochloride. As a non-limiting example, the polypeptides of the disclosure may include 8M urea and/or guanidium hydrochloride. In some embodiments, the polypeptides as described herein are stored in solutions or buffers containing 0.01-95 M, 0.01-90 M, 0.01-85 M, 0.01-80 M, 0.01-75 M, 0.01-70 M, 0.01-65 M, 0.01-60 M, 0.01-55 M, 0.01-50 M, 0.01-45 M, 0.01-40 M, 0.01-35 M, 0.01-30 M, 0.01-25 M, 0.01-20 M, 0.01-15 M, 0.01-10 M, 0.01-9 M, 0.01-8 M, 0.01-7 M, 0.01-6 M, 0.01-5 M, 0.01-4 M, 0.01-3 M, 0.01-2 M, 0.01-1 M, 0.01-0.9 M, 0.01-0.8 M, 0.01-0.7 M, 0.01-0.6 M, 0.01-0.5 M, 0.01-0.4 M, 0.01-0.3 M, 0.01-0.2 M, 0.01-0.1 M, 0.01-0.09 M, 0.01-0.08 M, 0.01-0.07 M, 0.01-0.06 M, 0.01-0.05 M, 0.01-0.04 M, 0.01-0.03 M, or 0.01-0.02 M guanidinium hydrochloride. In some embodiments, the polypeptides as described herein are stored in solutions or buffers containing 4-8 M, 3-9 M, 2-10 M, 1-15 M, 0.9-15 M, 0.8-20 M, 0.7-25 M, 0.6-30 M, 0.5-35 M, 0.4-40 M, 0.3-45 M, 0.2-50 M, 0.1-55 M, 0.09-60 M, 0.08-65 M, 0.07-70 M, 0.06-75 M, 0.05-80 M, 0.04-85 M, 0.03-90 M, or 0.02-95 M guanidinium hydrochloride. In some embodiments, the polypeptides as described herein are stored, for example, in buffers containing 4-8 M guanidinium hydrochloride.

In some embodiments, the polypeptides as described herein are stored in solutions or buffers containing 0.01-100 M, 0.05-100 M, 0.1-100 M, 0.5-100 M, 1-100 M, 2-100 M, 3-100 M, 4-100 M, 5-100 M, 6-100 M, 7-100 M, 8-100 M, 9-100 M, 10-100 M, 15-100 M, 20-100 M, 25-100 M, 30-100 M, 35-100 M, 40-100 M, 45-100 M, 50-100 M, 55-100 M, 60-100 M, 65-100 M, 70-100 M, 75-100 M, 80-100 M, 85-100 M, 90-100 M, or 95-100 M urea or guanidium hydrochloride with a salt such as but not limited to NaCl. In some embodiments, the polypeptides as described herein are stored in solutions or buffers containing 0.01-95 M, 0.01-90 M, 0.01-85 M, 0.01-80 M, 0.01-75 M, 0.01-70 M, 0.01-65 M, 0.01-60 M, 0.01-55 M, 0.01-50 M, 0.01-45 M, 0.01-40 M, 0.01-35 M, 0.01-30 M, 0.01-25 M, 0.01-20 M, 0.01-15 M, 0.01-10 M, 0.01-9 M, 0.01-8 M, 0.01-7 M, 0.01-6 M, 0.01-5 M, 0.01-4 M, 0.01-3M, 0.01-2 M, 0.01-1 M, 0.01-0.9 M, 0.01-0.8 M, 0.01-0.7 M, 0.01-0.6 M, 0.01-0.5 M, 0.01-0.4 M, 0.01-0.3 M, 0.01-0.2 M, 0.01-0.1 M, 0.01-0.09 M, 0.01-0.08 M, 0.01-0.07 M, 0.01-0.06 M, 0.01-0.05 M, 0.01-0.04 M, 0.01-0.03 M, or 0.01-0.02 M urea or guanidium hydrochloride with a salt such as but not limited to NaCl. In some embodiments, the polypeptides as described herein are stored in solutions or buffers containing 4-8 M, 3-9 M, 2-10 M, 1-15 M, 0.9-15 M, 0.8-20 M, 0.7-25 M, 0.6-30 M, 0.5-35 M, 0.4-40 M, 0.3-45 M, 0.2-50 M, 0.1-55 M, 0.09-60 M, 0.08-65 M, 0.07-70 M, 0.06-75 M, 0.05-80 M, 0.04-85 M, 0.03-90 M, or 0.02-95 M urea or guanidium hydrochloride with a salt such as but not limited to NaCl. In some embodiments, the polypeptides as described herein are stored, for example, in solutions or buffers containing 4-8 M urea or guanidium hydrochloride with a salt.

In some embodiments, the polypeptides as described herein are solubilized in denaturing buffer when combined with a lipid. In some embodiments, the polypeptides as described herein are buffer exchanged into non-denaturing buffer prior to combination with a lipid.

To combine the polypeptides with a lipid, the polypeptides may be pre-reduced with a reducing agent, e.g., TCEP ((tris(2-carboxyethyl)phosphine), and excess TCEP may be removed with, e.g., a spin desalting column (e.g., Zeba spin desalting column), pre-equilibrated with denaturing buffer. In some embodiments, the concentration of each of the polypeptides may be measured with conventional methods, for example, with the bicinchoninic acid (BCA) assay. In some embodiments, if necessary, the polypeptides may be diluted to a working concentration of, e.g., 1-3 mg/mL in denaturing buffer. In some embodiments, a lipid solubilized in resuspension buffer and quantified with the appropriate enzymatic assay may be added to the denatured solution of polypeptides.

### Lipid Preparation

To prepare a lipoprotein system of the disclosure, the polypeptides of the disclosure can be combined with a lipid described herein. In some embodiments, lipoprotein systems may be prepared by combining the polypeptides of the disclosure with at least one lipid described herein. In some embodiments, to prepare lipoprotein system of the disclosure, the polypeptides of the disclosure can be combined with two different lipids described herein. In some embodiments, to prepare lipoprotein system of the disclosure, the polypeptides of the disclosure can be combined with three different lipids described herein. In some embodiments, to prepare lipoprotein system of the disclosure, the polypeptides of the disclosure can be combined with four different lipids described herein. In some embodiments, to prepare lipoprotein system of the disclosure, the polypeptides of the disclosure can be combined with three different lipids described herein. In some embodiments, to prepare lipoprotein system of the disclosure, the polypeptides of the disclosure can be combined with more than four different lipids described herein.

In some embodiments, lipids may be stored in chloroform or in other organic solvents. In some embodiments, more than one different lipid (such as 2, 3, 4, and more than 4 different lipids) may be used, wherein each of the lipids may be stored in chloroform or in other organic solvents separately and combined with polypeptides of the disclosure to prepare lipoprotein system of the disclosures.

In some embodiments, more than one different lipid (such as 2, 3, 4, and more than 4 different lipids) are used, and the lipids may be mixed and stored in a chloroform or in other organic solvents with an aliquot ratio. The lipid mixture may be held for a certain period of time (from about 1 hour to about 6 months) and combined with polypeptides of the disclosure to form lipoprotein system as needed.

In some embodiments, lipid stock solutions are dried down under a stream of nitrogen gas and under reduced pressure. In some embodiments, lipids may be stored as lyophilized powders. In some embodiments, lipids may be resuspended in aqueous buffers containing dialyzable detergents (for example, sodium cholate). In some embodiments, lipid solutions may be warmed and bath sonicated to facilitate resuspension. In some embodiments, lipid solutions may be dispersed with a probe sonicator to form liposomes. In some embodiments, liposomes may be formed by extrusion in the absence of detergent.

In some embodiments, purified lipids may be stored either as solids or as stock solutions in, e.g., anhydrous CHCl₃ in sealed glass vials, e.g., under a blanket of N2 at −20° C. In some embodiments, nonpolar molecules, such as vitamin E, may be also dissolved in CHCl₃ and added to the lipid mixture.

In some embodiments, lipids from natural sources may be stored as such stock solutions (e.g., total brain lipid extract). In some embodiments, the lipids may be extracted just prior to use with, e.g., the Bligh-Dyer method (2:1 ratio of CHCl3/MeOH) from natural sources (e.g., human plasma HDL). In some embodiments, the organic solvent in which lipids are stored or extracted may be evaporated, e.g., under a stream of N2, and residual solvent may be further removed by lyophilization or reduced pressure overnight. In some embodiments, the dried lipids are resuspended in lipid resuspension buffer (e.g., 20-100 mM MOPS, 50 mM NaCl, 2% cholate w/w, pH 7.4), which may be pre-warmed to, e.g., 55° C. to facilitate emulsion.

In some embodiments, lipid resuspension buffer may be pre-warmed to 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90° C.

In some embodiments, lipid solutions may be vortexed until transparent, sonicated in a bath sonicator, or sonicated with an immersion probe sonicator. In some embodiments, lipids may be quantified prior to assembly of lipoprotein system of the disclosure. In some embodiments, purified lipid components may be quantified using enzyme-coupled reactions with a standard curve, for example, total phosphatidylcholine, total phosphatidylethanolamine, total free cholesterol, total cholesteryl esters, total triglycerides, and others.

In some embodiments, systems of the disclosure may include cholesterol or cholesterol derivatives. Cholesterol may enhance the stability of the lipoprotein system by modulating membrane integrity and rigidity (see Hajj et al. Nat. Rev. Mater. 2, 17056 (2017), the contents of which are incorporated by reference in its entirety). The molecular geometry of cholesterol derivatives can further affect delivery efficacy and biodistribution of the lipoprotein system. For example, cholesterol analogues with C-24 alkyl phytosterols increase the in vivo delivery efficacy of lipid nanoparticle-mRNA formulations —the length of the hydrophobic tails of the cholesterol analogues, the flexibility of sterol rings and the polarity of hydroxy groups impact delivery efficacy (see Patel et al. Nat. Commun. 11, 983 (2020), the contents of which are incorporated by reference in its entirety). In addition, cholesterol derivatives may affect the shape of the lipoprotein system, e.g., adopting a polyhedral shape with multilamellarity and lipid partitioning (see Eygeris et al. Nano Lett. 20, 4543-4549 (2020), the contents of which are incorporated by reference in its entirety). Lipoprotein system of the disclosure containing cholesteryl oleate may show higher selectivity for liver endothelial cells than for hepatocytes (see Paunovska et al. ACS Nano 12, 8341-8349 (2018), the contents of which are incorporated by reference in its entirety). Moreover, oxidative modifications on the cholesterol tail may allow lipoprotein system of the disclosure to accumulate more in liver endothelial cells and Kupffer cells than in hepatocytes (Paunovska et al. Adv. Mater. 31, 1807748 (2019), the contents of which are herein incorporated by reference in their entirety).

### Lipid Phase Transition Temperatures

Phase transition temperatures of a lipid may govern its phase and thus impact lipoprotein system assembly. The phase transition temperature (Tₘ) of a lipid refers to the characteristic temperature required for the lipid to transform from an ordered gel phase to a disordered fluid phase. Tm may depend on the hydrocarbon length, unsaturation, charge, and/or headgroup species. In the ordered gel phase, the hydrocarbon chains may be fully extended and closely packed. In the disordered fluid state, the hydrocarbon chains may be randomly oriented. In most cases, though lipid molecules may assemble into a lipid bilayer in both gel and fluid phases, they are constrained in the bilayer plane with very low lipid mobility in gel phase whereas diffuse freely within the bilayer plane in fluid phase. For example, 1,2-distearoyl-sn-glycero-3-phosphocholine (DSPC), a phosphatidylcholine with saturated tails, has a melting temperature of about 54° C. and a cylindrical geometry that allows DSPC molecules to form a lamellar phase, which may stabilize the structure of lipoprotein system (Koltover et al. Science 281, 78-81 (1998), the contents of which are incorporated by reference in its entirety). DOPE is a phosphoethanol amine with two unsaturated tails, which has a melting temperature of about 30° C. and a conical shape. DOPE tends to adopt an inverted hexagonal H(II) phase, which may be used to destabilize endosomal membranes and facilitates endosomal escape of lipoprotein system of the disclosure. Kauffman et al. Nano Lett. 15, 7300-7306 (2015), the contents of which are incorporated by reference in its entirety.

In some embodiments, to prepare lipoprotein system of the disclosure, the phase transition temperatures of single lipid species alone as well as of each combination of lipids may be determined empirically.

In some embodiments, the phase transition temperature may be from −100° C. to 100° C. In some embodiments, the phase transition temperature may be −90° C. to 100° C., −80° C. to 100° C., −70° C. to 100° C., −60° C. to 100° C., −50° C. to 100° C., −40° C. to 100° C., −30° C. to 100° C., −20° C. to 100° C., −10° C. to 100° C., 0° C. to 100° C., 10° C. to 100° C., 20° C. to 100° C., 30° C. to 100° C., 40° C. to 100° C., 50° C. to 100° C., 60° C. to 100° C., 70° C. to 100° C., 80° C. to 100° C., and 90° C. to 100° C. In some embodiments, the phase transition temperature may be from −100° C. to 90° C. In some embodiments, the phase transition temperature may be −100° C. to 80° C., −100° C. to 70° C., −100° C. to 60° C., −100° C. to 50° C., −100° C. to 40° C., −100° C. to 30° C.,−100° C. to 20° C., −100° C. to 10° C., −100° C. to 0° C., −100° C. to −10° C., −100° C. to −20° C., −100° C. to −30° C.,−100° C. to −40° C., −100° C. to −50° C., −100° C. to −60° C., −100° C. to −70° C., −100° C. to −80° C., and −100° C. to−90° C. In some embodiments, the phase transition temperature may be from −80° C. to 4° C. In some embodiments, the phase transition temperature may be −70° C. to 4° C., −60° C. to 4° C., −50° C. to 4° C., −40° C. to 4° C., −30° C. to 4° C., −20° C. to 4° C., −10° C. to 4° C., −9° C. to 4° C., −8° C. to 4° C., −7° C. to 4° C., −6° C. to 4° C., −5° C. to 4° C., −4° C. to 4° C., −3° C. to 4° C., −2° C. to 4° C., −1° C. to 4° C., 0° C. to 4° C., 1° C. to 4° C., 2° C. to 4° C., and 3° C. to 4° C. In some embodiments, the phase transition temperature may be from about 4° C. to about 40° C. In some embodiments, the phase transition temperature may be about 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C. and/or 40° C.

Lipid phase transition temperature of a lipid described herein may be measured by any method known in the art such as differential scanning calorimetry (DSC) and nanoplasmonic sensing (NPS). When more than one lipid (such as 2, 3, 4, and more than 4 different lipids) is present, the phase transition temperature of each lipid in the mixture may be measured by any method known in the art such as differential scanning calorimetry (DSC) and nanoplasmonic sensing (NPS). In some embodiments, the Tm of POPC may be about −2° C. In some embodiments, the Tm of DMPC may be about 24° C. In some embodiments, the Tm of DPPC may be about 41° C. In some embodiments, the Tm of DOPC may be about −20° C. In some embodiments, the Tm of DMPS may be about 35° C. In some embodiments, the Tm of POPS may be about 14° C. In some embodiments, the Tm of DOPS may be about −11° C. In some embodiments, the Tm of POPG may be about −2° C. In some embodiments, the Tm of DOPG may be about −18° C. In some embodiments, the Tm of DMPG may be about 23° C. In some embodiments, the Tm of SM may be about 37° C.

### Synthesis of Lipoprotein Systems

In some embodiments, provided herein are methods of producing a lipoprotein system of the disclosure. Lipoprotein systems of the disclosure may be prepared by any methods known in the art. In some embodiments, these methods may be cell-based methods or cell-free methods.

In some embodiments, a lipoprotein system of the disclosure as described herein may be prepared by any methods known in the art including but not limited to mechanical dispersion method (e.g., sonication such as ultrasound, French pressure cell extrusion, high-pressure homogenization such as hot homogenization and cold homogenization, and membrane extrusion), solvent dispersion method (such as ether injection, ethanol injection, thin-film hydration, and reverse-phase evaporation), rapid mixing method (e.g., microfluidic mixing), and detergent removal method (such as dialysis) (Musielak et al. Materials 2022, 15, 682, the contents of which are incorporated by reference in its entirety).

In some embodiments, a lipoprotein system of the disclosure may be produced by thin-film hydration. In some embodiments, the sizes of lipoproteins may be further optimized by extrusion techniques described in MacLachlan et al. Antis. Drug Technol. 2, 237-270 (2007); the contents of which are herein incorporated by reference in its entirety.

In some embodiments, a lipoprotein system of the disclosure may be produced by rapid mixing method. In some embodiments, rapid mixing may be facilitated by microfluidic devices. In some embodiments, a lipoprotein system of the disclosure may be produced by microfluidic mixing described in Jahn et al. ACS Nano. 2010; 4: 2077-2087.

In some embodiments, a lipoprotein system of the disclosure may be produced by detergent removal. In some embodiments, detergents may be removed from lipoprotein system of the disclosure using dialysis.

### Assembly and Purification

In some embodiments, provided herein is a method of assembling or purifying a lipoprotein system of the disclosure.

In some embodiments, a lipoprotein system of the disclosure is assembled by combining any of the polypeptides as described herein with any lipid as described herein, optionally followed by dilution of or removal of detergent (for example, by dialysis). In some embodiments, a lipoprotein system of the disclosure is assembled by combining the polypeptides as described herein with a detergent-solubilized lipid, followed by dilution of or removal of detergent by dialysis. In embodiments, adsorbent agents (for example, SM-2 Bio-Beads) are added to the assembly mixture or to the dialysate. Exemplary embodiment of adsorbent agents, SM-2 Bio-Beads, are added to the assembly mixture or to the dialysate. In some embodiments, the detergents may be absent or may be diluted far below the critical micelle concentration. In some embodiments, a lipoprotein system of the disclosure may form spontaneously when combined with a lipid. In some embodiments, in the absence of detergent, the assembly mixture may be heated and cooled to above and below the phase transition temperature of the lipid in one or more cycles to promote assembly of lipoprotein system of the disclosure.

In some embodiments, a lipoprotein system of the disclosure is assembled during dialysis. In some embodiments, a lipoprotein system of the disclosure may be assembled during dialysis in a dialysis device. In some embodiments, lipoprotein systems of the disclosure may be purified using diafiltration to aid in lipoprotein assembly and purification.

In some embodiments, a lipoprotein system of the disclosure of the disclosure may be purified by exhaustive dialysis. In some embodiments, a lipoprotein system of the disclosure may be purified by chromatography (for example, size exclusion chromatography, ion exchange chromatography, or heparin affinity chromatography). In some embodiments, a lipoprotein system of the disclosure of the disclosure may be purified by size exclusion chromatography, ion exchange chromatography, or heparin affinity chromatography. In some embodiments, a lipoprotein system of the disclosure of the disclosure may be purified by size exclusion chromatography. In some embodiments, a lipoprotein system of the disclosure of the disclosure may be purified by ion exchange chromatography. In some embodiments, a lipoprotein system of the disclosure may be purified by heparin affinity chromatography.

In some embodiments, a lipoprotein system of the disclosure may be purified in appropriate volume dialysis chamber, for example, a 96-well dialysis plate for small-scale screening reactions, or, for example, dialysis cassettes for larger scale reactions. In some embodiments, detergent and denaturant may be removed by dialysis, with, e.g., 1, 2, or above 2 exchanges of dialysis buffer at a volume, e.g., at least 20-fold greater than the lipoprotein system's assembly reaction volume. In some embodiments, an adsorbent (e.g., SM-2 Biobeads) may be added to the dialysis buffer to facilitate complete removal of the cholate detergent. In some embodiments, complete removal of the detergent may be validated by an enzyme-coupled assay for total bile acids (e.g., BioVision). In some embodiments, for small-scale screening reactions, the assembly reaction may be directly analyzed, e.g., by native PAGE, and the ratio resulting in the most uniform lipoprotein system of the disclosure may be used in subsequent larger scale assemblies. In some embodiments, for lipoprotein system of the disclosure assembled at larger scales, a variety of techniques may be used to characterize lipoprotein system of the disclosure.

In some embodiments, a lipoprotein system of the disclosure may be assessed by methods including native PAGE, size exclusion chromatography with or without multi-angle light scattering, dynamic light scattering, electron microscopy, and mass spectrometry.

In some embodiments, a lipoprotein system of the disclosure may be sterile filtered and stored as aqueous solutions. In some embodiments, stabilizing additives may be added to the buffer and lipoprotein system of the disclosure may be lyophilized and stored as powders.

In some embodiments, the molar ratio of the polypeptides to lipid may be experimentally determined for each of the polypeptides and for each lipid composition. In some embodiments, to determine the ideal ratios, small-scale (e.g., 10-50 μg with respect to the polypeptides) lipoprotein assembly reactions may be conducted, first spanning a wide range of protein: lipid ratios (for example, 1:20 mol/mol to 1:200 mol/mol), followed by a narrower range of ratios as needed.

Non-limiting examples of protein:lipid ratio may be 1:30, 1:60, 1:90, 1:120, 1:150, 1:180, 1:210, 1:1, 1:2, 1:3, 1:4, 1:5, 1:6, 1:7, 1:8, 1:9, 1:10, 1:20, 1:40, 1:50, 1:70, 1:80, 1:100, 1:110, 1:130, 1:140, 1:160, 1:170, 1:190, 1:200, 1:220, 1:230, 1:240, 1:250, 1:300, 1:400, 1:500, 1:600, 1:700, 1:800, 1:900, 1:1000, 1:5000, 1:10000, 2:1, 2:3, 2:5, 2:7, 2:9, 3:1, 3:2, 3:4, 3:5, 3:7, 3:8, 3:10, 3:20, 3:40, 3:50, 3:70, 3:80, 3:100, 3:110, 3:130, 3:140, 3:160, 3:170, 3:190, 3:200, 3:220, 3:230, 3:250, 3:400, 3:500, 3:700, 3:800, 3:1000, 3:5000, 3:10000, 1:1, 2:1, 3:1, 4:1, 5:1, 6:1, 7:1, 8:1, 9:1, 5:2, 7:2, 9:2, 1:3, 2:3, 4:3, 5:3, 7:3, 8:3, 10:1, 20:1, 30:1, 40:1, 50:1, 60:1, 70:1, 80:1, 90:1, 10:3, 20:3, 40:3, 50:3, 70:3, 80:3, 100:1, 110:1, 120:1, 130:1, 140:1, 150:1, 160:1, 170:1, 180:1, 190:1, 200:1, 210:1, 220:1, 230:1, 240:1, 250:1, 300:1, 400:1, 500:1, 600:1, 700:1, 800:1, 900:1, 100:3, 110:3, 130:3, 140:3, 160:3, 170:3, 190:3, 200:3, 220:3, 230:3, 250:3, 400:3, 500:3, 700:3, 800:3, 1000:1, 5000:1, 1000:3, 5000:3, 10000:1, and/or 10000:3 mol/mol.

### Storage

In some embodiments, a lipoprotein system of the disclosure may be stored in aqueous solution. In some embodiments, a lipoprotein system of the disclosure may be stored in freezing storage. In some embodiments, a lipoprotein system of the disclosure may be stored in lyophilized storage. In some embodiments, a lipoprotein system of the disclosure may be stored in liquid nitrogen. In some embodiments, a lipoprotein system of the disclosure may be stored at about −80° C. to about room temperature. In some embodiments, a lipoprotein system of the disclosure may be stored at about −60° C. to about 20° C., about −40° C. to about 15° C., or about −20° C. to about 10° C. In some embodiments, a lipoprotein system of the disclosure may be stored at about −80° C. to about 4° C. In some embodiments, a lipoprotein system of the disclosure may be stored at about −60° C. to about 4° C., about −40° C. to about 4° C., about −20° C. to about 4° C., or about −15° C. to about 4° C. In some embodiments, a lipoprotein system of the disclosure may be stored at about 4° C. In some embodiments, a lipoprotein system of the disclosure may be stored up to 12 months. In some embodiments, a lipoprotein system of the disclosure may be stored up to 12, 9, 6, 3, or 2 months. In some embodiments, a lipoprotein system of the disclosure may be stored up to 6, 5, 4, 3, 2, or 1 week without changing its safety and efficacy. In some embodiments, lipoprotein systems of the disclosure may include a stabilizing agent such as a carbohydrate e.g., mannitol and/or xylitol when the lipoprotein systems are stored.

### Synthesis of Lipoprotein System with Payload

In some embodiments, a lipoprotein system containing a payload may be prepared one or more methods described herein such as, but not limited to, a) combining one or more payloads with a lipid of the disclosure to form a mixture of lipid(s) and payload(s); b) combining the mixture in a) with polypeptides of the disclosure to form the lipoprotein system; and/or optionally c) purifying the system.

In some embodiments, a lipoprotein system of the disclosure may be prepared by a) combining payloads with a lipid of the disclosure to assembly into a payload-loaded lipid nanoparticles; b) combining the payload-loaded lipid nanoparticle in a) with polypeptides of the disclosure to re-assemble and re-structure into the lipoprotein system containing payload; and/or optionally c) purifying the system.

In some embodiments, the lipoprotein system of the disclosure may be prepared by a) combining payloads with polypeptides of the disclosure to form a mixture of polypeptides and payloads; b) combining the mixture in a) with a lipid of the disclosure to form lipoprotein systems containing payload; and c) purifying the system.

In some embodiments, the lipoprotein systems can be prepared by a) combining payloads with polypeptides of the disclosure to form conjugates of polypeptides and payloads; b) combining the conjugates in a) with a lipid of the disclosure to form lipoprotein system containing payload; and c) purifying the system.

In some embodiments, the lipoprotein systems can be prepared by a) combining payloads with polypeptides and a lipid of the disclosure in one assembly reaction to form lipoprotein system containing payload; and b) purifying the system.

In some embodiments, lipoprotein systems containing nucleic acid payloads are prepared using reagents that are free of any nuclease contamination.

In some embodiments, finally concentration or encapsulation of payloads may be further increased by enrichment.

### Analysis of Lipoprotein Systems

In some embodiments, a lipoprotein system of the disclosure may be screened according to particle size, polydispersity, structure, zeta potential, and thermal stability.

In some embodiments, lipoprotein systems may be screened according to particle size, polydispersity, and structure using, but not limited to, dynamic light scattering (DLS), transmission electron microscopy (TEM), small-angle neutron or X-ray scattering (SANS and SAXS, respectively), nuclear magnetic resonance spectroscopy (NMR), and computational approaches.

In some embodiments, a lipoprotein system of the disclosure may be screened according to thermal stability. In some embodiments, a lipoprotein system of the disclosure may be screened according to thermal stability by measuring phase transition temperatures thereof. In some embodiments, a lipoprotein system of the disclosure may be screened according to thermal stability by measuring the phase transition temperatures thereof using differential scanning calorimetry (DSC). In some embodiments, thermal stability may be measured using dynamic light scattering technique which measures particle size and aggregation of molecules.

In some embodiments, lipoproteins may be screened by evaluating stability, cell toxicity, cellular uptake and trafficking, payloads loading, payloads delivery, lipid efflux, and/or brain uptake and distribution.

In some embodiments, the stability of lipoproteins may be assessed by measuring their aggregation propensity. In some embodiments, the stability of lipoproteins is assessed by measuring their aggregation propensity, for example, by heating and shaking in the presence of thioflavin T to detect formation of amyloids. In some embodiments, the stability of lipoproteins may be assessed by measuring their susceptibility to proteolysis. In some embodiments, the stability of lipoproteins may be assessed by measuring their susceptibility to proteolysis, for example, by co-incubation with a purified proteases such as α-chymotrypsin, neprilysin, or insulin-degrading enzyme or by incubation with cells, tissues, or plasma and analyzed by SDS-PAGE to detect formation of proteolyzed fragments. In some embodiments, the stability of lipoprotein systems may be assessed by measuring their susceptibility to proteolysis, for example, by co-incubation with purified α-chymotrypsin, neprilysin, or insulin-degrading enzyme. In some embodiments, the stability of lipoproteins may be assessed by measuring their susceptibility to proteolysis, for example, by incubation with cells, tissues, or plasma and analyzed by SDS-PAGE to detect formation of proteolyzed fragments. In some embodiments, the stability of lipoproteins may be assessed in acidified solutions mimicking endo-lysosomal cellular compartments.

In some embodiments, the cell toxicity of lipoproteins may be measured by CellTiter Glo® (Madison, WI) and/or MTT assays. Cell toxicity may be measured using propidium iodide in conjunction with a nuclear stain e.g., Hoechst or DAPI. In some embodiments, the cellular uptake of lipoproteins may be measured by antibody-based detection. In some embodiments, the polypeptides as described herein are labeled with a fluorophore or an affinity handle and uptake may be directly or indirectly determined by measuring fluorescence intensity. In some embodiments, a fluorescent probe is incorporated into the lipoproteins. In some embodiments, lipoprotein systems may be chemically crosslinked to the cell surface (for example, with a cell surface biotinylation assay). In some embodiments, cell uptake may be measured in the presence or absence of lipoprotein receptors or in the presence or absence of cell surface modifications such as heparin.

In some embodiments, cell uptake may be measured in the presence or absence of cellular perturbagens, such as small molecules that block endocytosis or antibodies against specific lipoprotein receptors. In some embodiments, cell uptake may be measured in the presence of cellular perturbagens. In some embodiments, cell uptake may be measured in the presence of cellular perturbagens, such as small molecules that block endocytosis or antibodies against specific lipoprotein receptors.

In some embodiments, the efflux of lipids from cells or tissues may be measured by enzymatic assays or by mass spectrometry. In some embodiments, cells or tissues are pre-treated with a fluorescent probe (e.g., 25-NBD Cholesterol (also herein 25-[N-[(7-nitro-2-1,3-benzoxadiazol-4-yl)methyl]amino]-27-norcholesterol), and transfer of fluorescence to lipoproteins systems may be measured. In some embodiments, cells or tissues may be pre-treated with a fluorescent probe. In some embodiments, the efflux of lipids may be measured using fluorescent probes e.g., Filipin III which measures free cholesterol. The cells may be treated with Filipin III and fluorescent staining may be evaluated using fluorescence microscopy. A reduction in fluorescence intensity may be indicative of increased cholesterol efflux from the cells.

In some embodiments, the delivery of may be measured by enzymatic assays or by mass spectrometry. In some embodiments, lipoproteins may be loaded with a fluorescent probe, and transfer of fluorescence to cells or tissues may be measured. In some embodiments, lipoproteins may be loaded with a fluorescent probe. For example, lymphatic trafficking of lipid nanoparticles labeled with fluorophores may be tracked at a subcellular level in real-time with high resolution (see Cordeiro et al. J. Drug Target. 2019; 27: 646-658; the contents of which are herein incorporated by reference in its entirety). In some embodiments, positron emission tomography (PET) may be used to monitor lipoprotein systems in vivo. For example, PET-mediated trafficking of mRNA vaccines revealed the spatiotemporal trafficking of the vaccines in non-human primates (see Lindsay et al. Nat. Biomed. Eng. 2019; 3: 371-380 (the contents of which are herein incorporated by reference in its entirety). In some embodiments, visualization of lipoprotein system of the disclosure in cells and tissues at nanoscale may be achieved by in situ hybridization. For example, single molecule fluorescent in situ hybridization (smFISH) enables the delivered mRNA molecules to be detectable and quantifiable in fixed cells (Sabnis et al. Mol. Ther. 2018; 26: 1509-1519; the contents of which are herein incorporated by reference in its entirety). RNAScope® utilizes target-specific double Z probes, preamplifiers, and fluorophore-conjugated amplifiers in order to increase the signal-to-noise ratio for nanoparticle detection at specific tissues. For example, RNAScope® was recently used to visualize synthetic mRNA delivered by lipid nanoparticles to mouse liver at wide field-of-view.

In some embodiments, the compositions are screened according to the pipeline of evaluating stability, cell toxicity, cellular uptake and trafficking, lipid efflux, and brain uptake and distribution. In some embodiments, the compositions are screened according to the pipeline of evaluating stability, cell toxicity, cellular uptake and trafficking, cargo delivery, and brain uptake and distribution.

In some embodiments, the stability of the compositions is assessed by measuring their aggregation propensity. In some embodiments, the stability of the compositions is assessed by measuring their aggregation propensity, for example, by heating and shaking in the presence of thioflavin T to detect formation of amyloids. In some embodiments, the stability of the compositions is assessed by measuring their susceptibility to proteolysis. In some embodiments, the stability of the compositions is assessed by measuring their susceptibility to proteolysis, for example, by co-incubation with a purified proteases such as α-chymotrypsin, neprilysin, or insulin-degrading enzyme or by incubation with cells, tissues, or plasma and analyzed by SDS-PAGE to detect formation of proteolyzed fragments. In some embodiments, the stability of the lipoprotein systems is assessed by measuring their susceptibility to proteolysis, for example, by co-incubation with purified α-chymotrypsin, neprilysin, or insulin-degrading enzyme. In some embodiments, the stability of the compositions is assessed by measuring their susceptibility to proteolysis, for example, by incubation with cells, tissues, or plasma and analyzed by SDS-PAGE to detect formation of proteolyzed fragments. In some embodiments, the stability of the compositions is assessed in acidified solutions mimicking endo-lysosomal cellular compartments.

In some embodiments, the cell toxicity of the compositions is measured by CellTiter Glo or MTT assays. In some embodiments, the cell toxicity of the compositions is measured by CellTiter Glo. In some embodiments, the cell toxicity of the compositions is measured by MTT assays. In some embodiments, the cell toxicity of the compositions is measured by CellTiter Glo and MTT assays. In some embodiments, the cell toxicity of the compositions is measured by LDH Glo.

In some embodiments, the cellular uptake of the compositions is measured by antibody-based detection. In some embodiments, the compositions described herein are post-translationally labeled with a fluorophore or an affinity handle and uptake will be directly or indirectly determined by measuring fluorescence intensity. In some embodiments, the compositions as described herein are post-translationally labeled with a fluorophore. In some embodiments, the compositions as described herein are post-translationally labeled with an affinity handle. In some embodiments, a fluorescent probe is incorporated into the lipoprotein systems. In some embodiments, the compositions are chemically crosslinked to the cell surface (for example, with a cell surface biotinylation assay). In some embodiments, cell uptake is measured in the presence or absence of lipoprotein receptors or in the presence or absence of cell surface modifications such as heparin. In some embodiments, cell uptake is measured in the presence of lipoprotein receptors. In some embodiments, cell uptake is measured in the presence absence of lipoprotein receptors. In some embodiments, cell uptake is measured in the presence of cell surface modifications. In some embodiments, cell uptake is measured in the presence of cell surface modifications, such as heparin. In some embodiments, cell uptake is measured in the absence of cell surface modifications. In some embodiments, cell uptake is measured in the absence of cell surface modifications such as heparin.

In some embodiments, cell uptake is measured in the presence or absence of cellular perturbagens, such as small molecules that block endocytosis or antibodies against specific lipoprotein receptors. In some embodiments, cell uptake is measured in the presence of cellular perturbagens. In some embodiments, cell uptake is measured in the presence of cellular perturbagens, such as small molecules that block endocytosis or antibodies against specific lipoprotein receptors. In some embodiments, cell uptake is measured in the presence of small molecules that block endocytosis. In some embodiments, cell uptake is measured in the presence of antibodies against specific lipoprotein receptors. In some embodiments, cell uptake is measured in the absence of cellular perturbagens. In some embodiments, cell uptake is measured in the absence of cellular perturbagens, such as small molecules that block endocytosis or antibodies against specific lipoprotein receptors. In some embodiments, cell uptake is measured in the absence of cellular small molecules that block endocytosis. In some embodiments, cell uptake is measured in the absence of antibodies against specific lipoprotein receptors.

In some embodiments, the efflux of lipids from cells or tissues to the compositions is measured by enzymatic assays or by mass spectrometry. In some embodiments, the efflux of lipids from cells or tissues to the compositions is measured by enzymatic assays. In some embodiments, the efflux of lipids from cells or tissues to the compositions is measured by mass spectrometry. In some embodiments, cells or tissues are pre-treated with a fluorescent probe, and transfer of fluorescence to the compositions is measured. In some embodiments, cells or tissues are pre-treated with a fluorescent probe.

In some embodiments, the delivery of lipids or cargo to cells or tissues from the compositions is measured by enzymatic assays or by mass spectrometry. In some embodiments, the delivery of lipids or cargo to cells or tissues from the compositions is measured by enzymatic assays. In some embodiments, the delivery of lipids or cargo to cells or tissues from the compositions is measured by mass spectrometry. In some embodiments, the compositions are loaded with a fluorescent probe, and transfer of fluorescence to cells or tissues is measured. In some embodiments, the compositions are loaded with a fluorescent probe.

In some embodiments, the compositions are delivered orally, intranasal, intravenously, intraperitoneally, subcutaneously, or intrathecally. In some embodiments, the biodistribution of the compositions is measured by antibody-based detection. In some embodiments, the compositions as described herein are post-translationally labeled with a fluorophore or an affinity handle and biodistribution is directly or indirectly determined by measuring fluorescence, absorbance, or chemiluminescence intensity. In some embodiments, the compositions as described herein are post-translationally labeled with a fluorophore. In some embodiments, the compositions as described herein are post-translationally labeled with an affinity handle. In some embodiments, biodistribution of the compositions as described herein is directly determined by measuring fluorescence, absorbance, or chemiluminescence intensity. In some embodiments, biodistribution of the compositions as described herein is indirectly determined by measuring fluorescence, absorbance, or chemiluminescence intensity.

### Pharmaceutical Compositions

In some embodiments, provided herein is a pharmaceutical composition comprising the composition as described herein, including the lipoprotein systems as described herein. Pharmaceutical compositions described herein may further include one or more pharmaceutically or physiologically acceptable carriers, diluents, or excipients. Pharmaceutical compositions may include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

A pharmaceutical composition used in the therapeutic methods of the disclosure is formulated to be compatible with its intended route of administration.

For oral administration, the compositions may be formulated in liquid or solid dosage forms and as instant or controlled/sustained release formulations. Suitable dosage forms for oral ingestion by a subject include powders, tablets, pills, granules, dragees, hard- and soft-shell capsules, liquids, gels, syrups, slurries, suspensions, emulsions and the like. Oral compositions generally include an inert diluent or an edible carrier. They can be enclosed in gelatin capsules or compressed into tablets. For the purpose of oral therapeutic administration, the active agent can be incorporated with excipients and used in the form of tablets, troches, or capsules. Oral compositions may also be prepared using a fluid carrier for use as a mouthwash, wherein the agent in the fluid carrier is applied orally and swished and expectorated or swallowed. Pharmaceutically compatible binding agents, and/or adjuvant materials can be included as part of the composition. The tablets, granules, pills, capsules, troches and the like can contain any of the following ingredients, or compounds of a similar nature: a binder such as micro-crystalline cellulose, gum tragacanth or gelatin; an excipient such as starch or lactose; dissolution retardant; anti-adherents; cationic exchange resin; wetting agents; antioxidants; preservatives; a disintegrating agent such as alginic acid, Primogel, or corn starch; a lubricant such as magnesium stearate; a glidant such as colloidal silicon dioxide; a preservative; a colorant; a sweetening agent such as sugars such as dextrose, sucrose or saccharin; or a flavoring agent such as peppermint, methyl salicylate, or orange flavoring, each of these being synthetic and/or natural.

The terms “pharmaceutical composition” and “pharmaceutical formulation” (or “formulation”) may be used interchangeably and denote a mixture or solution comprising a therapeutically effective amount of an active pharmaceutical ingredient together with one or more pharmaceutically acceptable excipients to be administered to a subject, e.g., a human in need thereof. The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained. The terms “pharmaceutically acceptable excipient,” “pharmaceutically acceptable carrier,” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, excipients, preservatives or lubricants used in formulating pharmaceutical products.

In some embodiments, the compositions are prepared with carriers that may protect the components of the composition against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions (including lipoprotein systems targeted to infected cells with monoclonal antibodies to viral antigens) can also be used as pharmaceutically acceptable carriers. These can be prepared according to methods known to those skilled in the art, for example, as described in U.S. Pat. No. 4,522,811.

For administration by inhalation, e.g., intranasal administration, the compositions are delivered in the form of an aerosol spray from pressured container or dispenser which contains a suitable propellant, e.g., a gas such as carbon dioxide, or a nebulizer. Systemic administration can also be by transmucosal or trans-dermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration may be accomplished through the use of nasal sprays or suppositories. For transdermal administration, the active agents are formulated into ointments, salves, gels, or creams, emulsion, a solution, a suspension, or a foam, as generally known in the art. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustments; use of complexing agents and other techniques, such as iontophoresis, may be used to regulate skin penetration of the active ingredient.

The compositions may also be formulated in rectal compositions, such as suppositories (e.g., with conventional suppository bases such as cocoa butter and other glycerides) or retention enemas.

Pharmaceutical compositions suitable for injectable use include sterile aqueous solutions (where water soluble) or dispersions and sterile powders for the extemporaneous preparation of sterile injectable solutions or dispersion. Solutions or suspensions used for parenteral, intranasal, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. For example, depending on the injection site, the vehicle may contain water, synthetic or vegetable oil, and/or organic co-solvents. In certain instances, such as with lyophilized product or a concentrate, the parenteral formulation would be reconstituted or diluted prior to administration. pH can be adjusted with acids or bases, such as hydrochloric acid or sodium hydroxide. Depot formulations, providing controlled or sustained release of a composition of the disclosure, may include injectable suspensions of nano/micro particles or nano/micro or non-micronized crystals.

For intravenous administration, suitable carriers include physiological saline, bacteriostatic water, Cremophor EL™ (BASF, Parsippany, N.J.) or phosphate buffered saline (PBS). Pharmaceutical compositions must be stable under the conditions of manufacture and storage and must be preserved against the contaminating action of microorganisms such as bacteria and fungi. The carrier may be a solvent or dispersion medium containing, for example, water, ethanol, poly(ol) (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Sterile injectable solutions can be prepared by incorporating the composition in the required amount in an appropriate solvent with one or a combination of ingredients enumerated above, as required, followed by filtered sterilization. Prevention of the action of micro-organisms can be achieved by various antibacterial and antifungal agents, for example, parabens, chlorobutanol, phenol, ascorbic acid, thimerosal, and the like.

Generally, dispersions are prepared by incorporating the active composition into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In many cases, it will be preferable to include isotonic agents, for example, sugars, poly alcohols such as mannitol, sorbitol, and sodium chloride in the composition. Prolonged absorption of the injectable compositions can be brought about by including in the composition an agent which delays absorption, for example, aluminum monostearate and gelatin. The parenteral preparation can be enclosed in ampoules, disposable syringes or multiple dose vials made of glass or plastic.

Pharmaceutically or physiologically acceptable carriers, diluents or excipients include, but are not limited to, antifoaming agents, antioxidants, binders, carriers or carrier materials, dispersing agents, viscosity modulating agents, diluents, filling agents, lubricants, glidants, plasticizers, solubilizers, stabilizers, suspending agents, surfactants, viscosity enhancing agents, and wetting agents.

Components of the compositions of the disclosure may be preblended or each component may be added separately to the same environment according to a predetermined dosage for the purpose of achieving the desired concentration level of the treatment components and so long as the components eventually come into intimate admixture with each other. Further, the compositions of the present disclosure may be administered or delivered on a continuous or intermittent basis.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the compositions and the particular therapeutic effect to be achieved, and the limitations inherent in the art of compounding such an agent for the treatment of subjects.

In some embodiments, provided herein is a pharmaceutical composition comprising the composition as described herein. Pharmaceutical compositions described herein may further include one or more pharmaceutically or physiologically acceptable carriers, diluents, or excipients. Such compositions may include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

A pharmaceutical composition used in the therapeutic methods of the disclosure is formulated to be compatible with its intended route of administration.

For oral administration, the compositions can be formulated in liquid or solid dosage forms and as instant or controlled/sustained release formulations. Suitable dosage forms for oral ingestion by a subject include powders, tablets, pills, granules, dragees, hard- and soft-shell capsules, liquids, gels, syrups, slurries, suspensions, emulsions and the like. Oral compositions generally include an inert diluent or an edible carrier. They can be enclosed in gelatin capsules or compressed into tablets. For the purpose of oral therapeutic administration, the active agent can be incorporated with excipients and used in the form of tablets, troches, or capsules. Oral compositions can also be prepared using a fluid carrier for use as a mouthwash, wherein the agent in the fluid carrier is applied orally and swished and expectorated or swallowed. Pharmaceutically compatible binding agents, and/or adjuvant materials can be included as part of the composition. The tablets, granules, pills, capsules, troches and the like can contain any of the following ingredients, or compounds of a similar nature: a binder such as micro-crystalline cellulose, gum tragacanth or gelatin; an excipient such as starch or lactose; dissolution retardant; anti-adherents; cationic exchange resin; wetting agents; antioxidants; preservatives; a disintegrating agent such as alginic acid, Primogel, or corn starch; a lubricant such as magnesium stearate or Sterotes; a glidant such as colloidal silicon dioxide; a preservative; a colorant; a sweetening agent such as sugars such as dextrose, sucrose or saccharin; or a flavoring agent such as peppermint, methyl salicylate, or orange flavoring, each of these being synthetic and/or natural.

The terms “pharmaceutical composition” and “pharmaceutical formulation” (or “formulation”) are used interchangeably and denote a mixture or solution comprising a therapeutically effective amount of an active pharmaceutical ingredient together with one or more pharmaceutically acceptable excipients to be administered to a subject, e.g., a human in need thereof.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The terms “pharmaceutically acceptable excipient,” “pharmaceutically acceptable carrier,” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, excipients, preservatives or lubricants used in formulating pharmaceutical products.

In some embodiments, the compositions are prepared with carriers that will protect the components of the composition against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral anti-gens) can also be used as pharmaceutically acceptable carriers. These can be prepared according to methods known to those skilled in the art, for example, as described in U.S. Pat. No. 4,522,811.

The term “preparation,” is intended to include the formulation of the compositions with encapsulating material as a carrier providing a capsule in which the active compositions with or without other carriers, is surrounded by a carrier, which is thus in association with it.

For administration by inhalation, e.g., intranasal administration, the compositions are delivered in the form of an aerosol spray from pressured container or dispenser which contains a suitable propellant, e.g., a gas such as carbon dioxide, or a nebulizer. Systemic administration can also be by transmucosal or trans-dermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active agents are formulated into ointments, salves, gels, or creams, emulsion, a solution, a suspension, or a foam, as generally known in the art. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustments; use of complexing agents and other techniques, such as iontophoresis, may be used to regulate skin penetration of the active ingredient.

The compositions may also be formulated in rectal compositions, such as suppositories (e.g., with conventional suppository bases such as cocoa butter and other glycerides) or retention enemas.

Pharmaceutical compositions suitable for injectable use include sterile aqueous solutions (where water soluble) or dispersions and sterile powders for the extemporaneous preparation of sterile injectable solutions or dispersion. Solutions or suspensions used for parenteral, intranasal, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. For example, depending on the injection site, the vehicle may contain water, synthetic or vegetable oil, and/or organic co-solvents. In certain instances, such as with lyophilized product or a concentrate, the parenteral formulation would be reconstituted or diluted prior to administration. pH can be adjusted with acids or bases, such as hydrochloric acid or sodium hydroxide. Depot formulations, providing controlled or sustained release of a composition of the disclosure, may include injectable suspensions of nano/micro particles or nano/micro or non-micronized crystals.

For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, poly(ol) (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Sterile injectable solutions can be prepared by incorporating the composition in the required amount in an appropriate solvent with one or a combination of ingredients enumerated above, as required, followed by filtered sterilization. Prevention of the action of micro-organisms can be achieved by various antibacterial and antifungal agents, for example, parabens, chlorobutanol, phenol, ascorbic acid, thimerosal, and the like.

Generally, dispersions are prepared by incorporating the active composition into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin. The parenteral preparation can be enclosed in ampoules, disposable syringes or multiple dose vials made of glass or plastic.

Pharmaceutically or physiologically acceptable carriers, diluents or excipients include, but are not limited to, antifoaming agents, antioxidants, binders, carriers or carrier materials, dispersing agents, viscosity modulating agents, diluents, filling agents, lubricants, glidants, plasticizers, solubilizers, stabilizers, suspending agents, surfactants, viscosity enhancing agents, and wetting agents.

The separate components of the compositions of the disclosure may be preblended or each component may be added separately to the same environment according to a predetermined dosage for the purpose of achieving the desired concentration level of the treatment components and so long as the components eventually come into intimate admixture with each other. Further, the compositions of the present disclosure may be administered or delivered on a continuous or intermittent basis.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the compositions and the particular therapeutic effect to be achieved, and the limitations inherent in the art of compounding such an agent for the treatment of subjects.

In some embodiments, provided herein is a method of producing the pharmaceutical composition as described herein.

Pharmaceutical compositions can be formulated in a conventional manner using one or more pharmaceutically acceptable inactive ingredients that facilitate processing of the active compounds into preparations that can be used pharmaceutically. A proper formulation is dependent upon the route of administration chosen and a summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference. In some embodiments, the pharmaceutical composition facilitates administration of the compound to an organism.

### Dosing, Administration and Delivery

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Adjustment and manipulation of established dosages (e.g., frequency and duration) are well within the ability of those skilled in the art. The treatment, such as those disclosed herein, can be administered to the subject on a daily, twice daily, biweekly, monthly or any applicable basis that is therapeutically effective. In some embodiments, the treatment is only on an as-needed basis, e.g., upon appearance of signs or symptoms of a condition.

The skilled artisan will appreciate that certain factors may influence the dosage and frequency of administration required to effectively treat a subject, including but not limited to the severity of the disease or disorder, previous treatments, the general characteristics of the subject including health, sex, weight and/or age of the subject, and other diseases present. Moreover, treatment of a subject with a therapeutically effective amount of the compositions can include a single treatment or, preferably, can include a series of treatments. It will also be appreciated that the effective dosage of the composition of the disclosure used for treatment may increase or decrease over the course of a particular treatment. Changes in dosage may result and become apparent from the results of diagnostic assays as described herein. The therapeutically effective dosage will generally be dependent on the patient's status at the time of administration. The precise amount can be determined by routine experimentation.

The compositions of the present disclosure can be administered to a subject using any suitable methods known in the art. Suitable formulations for use with the compositions of the present disclosure and methods of delivery are generally well known in the art. For example, the compositions described herein can be administered to the subject in a variety of ways, including parenterally, intracranial, intrathecal, intravenously, intradermally, intramuscularly, rectally, or intraperitoneally. In some embodiments, the compositions described herein are administered by intracranial injection, intrathecal injection, intraperitoneal injection, intramuscular injection, subcutaneous injection, or intravenous injection of the subject. In some embodiments, the compositions described herein can be administered intravenously, intracranially, intrathecally, or intranasally.

For some embodiments where a prophylactic benefit is desired, a pharmaceutical composition of the disclosure can be administered to a subject at risk of developing a condition, or to a subject reporting one or more of the physiological symptoms of a condition, even though a screening of the condition cannot have been made. Administration can prevent a condition from developing, or it can reduce, lessen, shorten and/or otherwise ameliorate the progression of a condition, or symptoms that develop. The pharmaceutical composition can modulate or target a condition associated biomarker. Wherein, the term modulate includes inhibition of a condition associated biomarker or alternatively activation of a condition associated biomarker.

Reducing the activity of one or more condition's associated biomarkers is also referred to as “inhibiting” the condition's associated biomarkers. The term “inhibits” and its grammatical conjugations, such as “inhibitory,” do not require complete inhibition, but refer to a reduction in a condition's associated biomarkers' activities. In some embodiments such reduction is by at least 5%, at least 10%, at least 20%, at least 30%, at least 40%, at least 50%, at least 75%, at least 90%, and can be by at least 95% of the activity of the enzyme or other biologically important molecular process in the absence of the inhibitory effect, e.g., in the absence of an inhibitor. Increasing the activity and/or function of polypeptides and/or nucleic acids found to be associated with one or more conditions, is also referred to as “activating” the polypeptides and/or nucleic acids. The term “activated” and its grammatical conjugations, such as “activating,” do not require complete activation, but refer to an increase in the activity of one or more biomarkers. In some embodiments such increase is by at least 5%, at least 10%, at least 20%, at least 30%, at least 40%, at least 50%, at least 60%, at least 70%, at least 75%, at least 80%, at least 85%, at least 90%, and can be by at least 95% of the activity of the enzyme or other biologically important molecular process in the absence of the activation effect, e.g., in the absence of an activator. The ability to reduce enzyme activity is a measure of the potency or the activity of an agent, or combination of agents, towards or against the enzyme or other biologically important molecular processes. Potency can be measured by cell free, whole cell and/or in vivo assays in terms of IC50, Ki and/or ED50 values. An IC50 value represents the concentration of an agent required to inhibit enzyme activity by half (50%) under a given set of conditions. A Ki value represents the equilibrium affinity constant for the binding of an inhibiting agent to the enzyme or other relevant biomolecule. An ED50 value represents the dose of an agent required to affect a half-maximal response in a biological assay. Further details of these measures will be appreciated by those of ordinary skill in the art, and can be found in standard texts on biochemistry, enzymology, and the like.

A patient who is being treated for a condition is one who a medical practitioner has diagnosed as having such a condition. Diagnosis may be by any suitable means. Diagnosis and monitoring may involve, for example, detecting the presence of destroyed or dying cells in a biological sample (e.g., tissue biopsy, blood test, or urine test), detecting the presence of plaques, detecting the level of a surrogate marker of the condition in a biological sample, or detecting symptoms associated with the condition. A patient in whom the development of a condition is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition). Conditions include diseases, disorders, and susceptibilities.

Toxicity and therapeutic efficacy of the compositions of the disclosure can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects (the ratio LD50/ED50) is the therapeutic index. Agents that exhibit high therapeutic indices are preferred. The dosage of agents lies preferably within a range of circulating concentrations that include the ED50 with little or no toxicity. While agents that exhibit toxic side effects may be used, care should be taken to design a delivery system that targets such agents to the site of affected tissue in order to minimize potential damage to uninfected cells and, thereby, reduce side effects.

The methods and compositions of the disclosure described herein including embodiments thereof can be administered with one or more additional therapeutic regimens or agents or treatments, which can be co-administered to the mammal. By “co-administering” is meant administering one or more additional therapeutic regimens or agents or treatments and the composition of the disclosure sufficiently close in time to enhance the effect of one or more additional therapeutic agents, or vice versa. In this regard, the composition of the disclosure described herein can be administered simultaneously with one or more additional therapeutic regimens or agents or treatments, at a different time, or on an entirely different therapeutic schedule (e.g., the first treatment can be daily, while the additional treatment is weekly). For example, in some embodiments, the secondary therapeutic regimens or agents or treatments are administered simultaneously, prior to, or subsequent to the composition of the disclosure.

### Dosing

The present disclosure provides methods of administering the lipoprotein systems of the disclosure to a subject in need thereof. These may be administered to a subject using any amount and any route of administration effective for preventing or treating or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like. The term “subject” or “patient” encompasses vertebrates or mammals. Mammals include, but are not limited to humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein includes human beings and non-human animals. In some embodiments, a “non-human animal” is a mammal, for example, a rodent such as rat or a mouse. In some embodiments, a non-human animal is a mouse. A “control” is an alternative subject or sample used in an experiment for the purpose of comparison. A control can be “positive” or “negative.” Compositions in accordance with the disclosure are typically formulated in dosage unit form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

### Administration

The pharmaceutical compositions or the lipoprotein systems of the present disclosure may be administered by any route to achieve a therapeutically effective outcome. These include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

### Parenteral and Injectable Administration

The pharmaceutical compositions or the lipoprotein systems of the present disclosure may be administered parenterally. Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may include inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropylcellulose.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered compositions is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Rectal and Vaginal Administration

The pharmaceutical compositions or the lipoprotein systems of the present disclosure may be administered rectally and/or vaginally. Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

### Oral Administration

In some embodiments, pharmaceutical compositions or the lipoprotein systems of the present disclosure may be administered orally. Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g., starches, lactose, sucrose, glucose, mannitol, and silicic acid), binders (e.g., carboxymethylcellulose, alginates, gelatin, polyvinylpyrrolidinone, sucrose, and acacia), humectants (e.g., glycerol), disintegrating agents (e.g., agar, calcium carbonate, potato or tapioca starch, alginic acid, certain silicates, and sodium carbonate), solution retarding agents (e.g., paraffin), absorption accelerators (e.g., quaternary ammonium compounds), wetting agents (e.g., cetyl alcohol and glycerol monostearate), absorbents (e.g., kaolin and bentonite clay), and lubricants (e.g., talc, calcium stearate, magnesium stearate, solid polyethylene glycols, sodium lauryl sulfate), and mixtures thereof. In the case of capsules, tablets and pills, the dosage form may include buffering agents.

### Topical or Transdermal Administration

As described herein, the pharmaceutical compositions or the lipoprotein systems of the present disclosure may be formulated for administration topically. The skin may be an ideal target site for delivery as it is readily accessible. Three routes are commonly considered to deliver the pharmaceutical compositions or the lipoprotein systems of the present disclosure to the skin: (i) topical application (e.g., for local/regional treatment and/or cosmetic applications); (ii) intradermal injection (e.g., for local/regional treatment and/or cosmetic applications); and (iii) systemic delivery (e.g., for treatment of dermatologic diseases that affect both cutaneous and extracutaneous regions). Pharmaceutical compositions, or the lipoprotein systems of the present disclosure can be delivered to the skin by several different approaches known in the art.

In some embodiments, the disclosure provides for a variety of dressings (e.g., wound dressings) or bandages (e.g., adhesive bandages) for conveniently and/or effectively carrying out methods of the present disclosure. Typically dressing or bandages may include sufficient amounts of the pharmaceutical compositions or the lipoprotein systems of the present disclosure described herein to allow users to perform multiple treatments.

Dosage forms for topical and/or transdermal administration may include ointments, pastes, creams, lotions, gels, powders, solutions, sprays, inhalants and/or patches. Generally, active ingredients are admixed under sterile conditions with pharmaceutically acceptable excipients and/or any needed preservatives and/or buffers. Additionally, the present disclosure contemplates the use of transdermal patches, which often have the added advantage of providing controlled delivery of the pharmaceutical compositions or the lipoprotein systems of the present disclosure to the body. Such dosage forms may be prepared, for example, by dissolving and/or dispensing the pharmaceutical compositions or the lipoprotein systems of the present disclosure in the proper medium. Alternatively, or additionally, rates may be controlled by either providing rate controlling membranes and/or by dispersing pharmaceutical compositions or the lipoprotein systems of the present disclosure in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically administrable formulations may, for example, include from about 1% to about 10% (w/w) active ingredient, although the concentration of active ingredient may be as high as the solubility limit of the active ingredient in the solvent. Formulations for topical administration may further include one or more of the additional ingredients described herein.

### Depot Administration

In some embodiments, the pharmaceutical compositions or the lipoprotein systems of the present disclosure are formulated in depots for extended release. Generally, specific organs or tissues (“target tissues”) are targeted for administration.

In some aspects of the disclosure, the pharmaceutical compositions or the lipoprotein systems of the present disclosure are spatially retained within or proximal to target tissues. Provided are method of providing pharmaceutical compositions or the lipoprotein systems of the present disclosure to target tissues of mammalian subjects by contacting target tissues (which include one or more target cells) with pharmaceutical compositions, or the lipoprotein systems under conditions such that they are substantially retained in target tissues, meaning that at least 10, 20, 30, 40, 50, 60, 70, 80, 85, 90, 95, 96, 97, 98, 99, 99.9, 99.99 or greater than 99.99% of the composition is retained in the target tissues. Advantageously, retention is determined by measuring the amount of pharmaceutical compositions, or the lipoprotein systems that enter one or more target cells. For example, at least 1%, 5%, 10%, 20%, 30%, 40%, 50%, 60%, 70%, 80%, 85%, 90%, 95%, 96%, 97%, 98%, 99%, 99.9%, 99.99% or greater than 99.99% of pharmaceutical compositions, or lipoprotein systems administered to subjects are present intracellularly at a period of time following administration. For example, intramuscular injection to mammalian subjects may be performed using aqueous compositions comprising pharmaceutical compositions, or lipoprotein systems of the present disclosure and one or more transfection reagent, and retention is determined by measuring the amount of pharmaceutical compositions, or lipoprotein systems present in muscle cells.

Certain aspects of the disclosure are directed to methods of providing pharmaceutical compositions or the lipoprotein systems of the present disclosure to a target tissues of mammalian subjects, by contacting target tissues (comprising one or more target cells) pharmaceutical compositions, or the lipoprotein systems under conditions such that they are substantially retained in such target tissues. Pharmaceutical compositions, or the lipoprotein systems of the present disclosure include enough active ingredient such that the effect of interest is produced in at least one target cell. In some embodiments, pharmaceutical compositions or the lipoprotein systems of the present disclosure generally include one or more cell penetration agents, although “naked” formulations (such as without cell penetration agents or other agents) are also contemplated, with or without pharmaceutically acceptable carriers.

In some embodiments, the amount of a growth factor present in cells in a tissue is desirably increased. Preferably, this increase in growth factor is spatially restricted to cells within the target tissue. Thus, provided are methods of increasing the amount of growth factor of interest in tissues of mammalian subjects. In some embodiments, formulations are provided comprising pharmaceutical compositions or the lipoprotein systems of the present disclosure characterized in that the unit quantity provided has been determined to produce a desired level of growth factor of interest in a substantial percentage of cells contained within predetermined volumes of target tissue.

In some embodiments, formulations include a plurality of different pharmaceutical compositions or the lipoprotein systems of the present disclosure, with one or more payloads. In such embodiments, determinations are made of compound and/or composition dose required to target biomolecules of interest in substantial percentages of cells contained within predetermined volumes of the target tissue (generally, without targeting biomolecules of interest in adjacent or distal tissues). Determined doses are then introduced directly into subject tissues. In some embodiments, the disclosure provides for pharmaceutical compositions, or the lipoprotein systems to be delivered in more than one administration or by split dose administration.

### Pulmonary Administration

In some embodiments pharmaceutical compositions or the lipoprotein systems of the present disclosure may be prepared, packaged, and/or sold in formulations suitable for pulmonary administration. In some embodiments, such administration is via the buccal cavity. In some embodiments, formulations may include dry particles comprising active ingredients. In such embodiments, dry particles may have a diameter in the range from about 0.5 nm to about 7 nm or from about 1 nm to about 6 nm. In some embodiments, formulations may be in the form of dry powders for administration using devices comprising dry powder reservoirs to which streams of propellant may be directed to disperse such powder. In some embodiments, self-propelling solvent/powder dispensing containers may be used. In such embodiments, active ingredients may be dissolved and/or suspended in low-boiling propellant in sealed containers. Such powders may include particles wherein at least 98% of the particles by weight have diameters greater than 0.5 nm and at least 95% of the particles by number have diameters less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally, propellants may constitute 50% to 99.9% (w/w) of the composition, and active ingredients may constitute 0.1% to 20% (w/w) of the composition. Propellants may further include additional ingredients such as liquid non-ionic and/or solid anionic surfactant and/or solid diluent (which may have particle sizes of the same order as particles comprising active ingredients).

Pharmaceutical compositions formulated for pulmonary delivery may provide active ingredients in the form of droplets of solution and/or suspension. Such formulations may be prepared, packaged, and/or sold as aqueous and/or dilute alcoholic solutions and/or suspensions, optionally sterile, comprising active ingredients, and may conveniently be administered using any nebulization and/or atomization device. Such formulations may further include one or more additional ingredients including, but not limited to, a flavoring agent such as saccharin sodium, a volatile oil, a buffering agent, a surface-active agent, and/or a preservative such as methylhydroxybenzoate. Droplets provided by this route of administration may have an average diameter in the range from about 0.1 nm to about 200 nm.

### Intranasal and Buccal Administration

In some embodiments, pharmaceutical compositions or the lipoprotein systems of the present disclosure may be administered nasally. In some embodiments, formulations described herein as being useful for pulmonary delivery may also be useful for intranasal delivery. In some embodiments, formulations for intranasal administration may include a coarse powder comprising the active ingredient and having an average particle from about 0.2 mm to 500 mm. Such formulations are administered in the manner in which snuff is taken, i.e., by rapid inhalation through the nasal passage from a container of the powder held close to the nose.

Formulations suitable for nasal administration may, for example, include from about as little as 0.1% (w/w) and as much as 100% (w/w) of active ingredient, and may include one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may include powders and/or an aerosolized and/or atomized solutions and/or suspensions comprising active ingredients. Such powdered, aerosolized, and/or aerosolized formulations, when dispersed, may include average particle and/or droplet sizes in the range of from about 0.1 nm to about 200 nm, and may further include one or more of any additional ingredients described herein.

### Ophthalmic or Otic Administration

In some embodiments, pharmaceutical compositions or the lipoprotein systems of the present disclosure may be prepared, packaged, and/or sold in formulations suitable for ophthalmic and/or otic administration. Such formulations may, for example, be in the form of eye and/or ear drops including, for example, a 0.1/1.0% (w/w) solution and/or suspension of the active ingredient in aqueous and/or oily liquid excipients. Such drops may further include buffering agents, salts, and/or one or more other of any additional ingredients described herein. Other ophthalmically-administrable formulations which are useful include those which include active ingredients in microcrystalline form and/or in liposomal preparations. Subretinal inserts may also be used as forms of administration.

### Delivery

### Naked Delivery

Pharmaceutical compositions, or the lipoprotein systems of the present disclosure may be delivered to cells, tissues, organs and/or organisms in naked form. As used herein in, the term “naked” refers to pharmaceutical compositions, or the lipoprotein systems delivered free from agents or modifications which promote permeability. The naked pharmaceutical compositions may be delivered to the cells, tissues, organs and/or organisms using routes of administration known in the art and described herein. In some embodiments, naked delivery may include formulation in a simple buffer such as saline or PBS.

### Formulated Delivery

In some embodiments, pharmaceutical compositions may be formulated, using methods described herein. Formulations may include pharmaceutical compositions, or the lipoprotein systems which may be modified and/or unmodified. Formulations may further include, but are not limited to, cell penetration agents, pharmaceutically acceptable carriers, delivery agents, bioerodible or biocompatible polymers, solvents, and/or sustained-release delivery depots. Formulations of the present disclosure may be delivered to cells using routes of administration known in the art and described herein.

Pharmaceutical compositions, or the lipoprotein systems of the present disclosure may also be formulated for direct delivery to organs or tissues in any of several ways in the art including, but not limited to, direct soaking or bathing, via a catheter, by gels, powder, ointments, creams, gels, lotions, and/or drops, by using substrates such as fabric or biodegradable materials coated or impregnated with compositions, and the like.

### Methods and Uses

The pharmaceutical compositions, lipoprotein system, and polypeptides of the present disclosure may be utilized in a large variety of applications including, but not limited to, therapeutics, diagnostics, prognostic, and/or research applications.

The present disclosure provides a method for treating a disease, disorder and/or condition in a mammalian subject, including a human subject. Such methods may include administering to the subject the lipoprotein systems described herein or administering to the subject any of the described compositions, including pharmaceutical compositions, described herein.

In some embodiments, the lipoprotein system of the present disclosure is provided to a subject having at least one of the diseases described herein.

In some embodiments, provided herein is a method of reducing or preventing a cell death comprising, administering the compositions as described herein, wherein the administering may be effective to reduce or prevent cell death.

In some embodiments, administering the compositions as described herein reduces or prevents a cell death by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, at least 99.5%, at least 99.9%, or 100% as compared with an untreated control, or compared with the corresponding activity in the same type of cell before treatment with the lipoprotein system as described herein as measured by any standard technique. In some embodiments, administering the lipoprotein system as described herein reduces or prevents a cell death by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared with an untreated control, or compared with the corresponding activity in the same type of cell before treatment with the lipoprotein system as described herein as measured by any standard technique.

In some embodiments, administering the lipoprotein system as described herein reduces or prevents the death of lung cells, heart cells, brain cells, microglia, blood cells, stomach cells, liver cells, intestinal cells, pancreatic cells, colon cells, kidney cells, ureter cells, bladder cells, lymphatic cells, leukocytes, muscle cells, neuronal cells, macrophages, astrocytes, stromal cells, spinal cord cells, ovarian cells, vagina cells, prostate cells, bone cells, cartilage cells, ligament cells, or tendon cells.

In some embodiments, administering the lipoprotein system as described herein reduces or prevents apoptosis, necrosis, or ferroptosis.

The term “ferroptosis,” as used herein, refers to a type of programmed cell death dependent on iron and characterized by the accumulation of lipid peroxides. Ferroptosis is genetically and biochemically distinct from other forms of regulated cell death, such as apoptosis. In some embodiments, ferroptosis is initiated by the failure of the glutathione-dependent antioxidant defenses, resulting in unchecked lipid peroxidation and eventual cell death. In some embodiments, some of the molecules that regulate ferroptosis are involved in metabolic pathways that regulate, e.g., cysteine exploitation, glutathione state, nicotinamide adenine dinucleotide phosphate function, lipid peroxidation and iron homeostasis. The term “apoptosis,” as used herein, refers to a form of programmed cell death that occurs in multicellular organisms. Examples of characteristic cell changes include blebbing, cell shrinkage, nuclear fragmentation, chromatin condensation, chromosomal DNA fragmentation, and mRNA decay. Apoptosis is a highly regulated and controlled process. The term “necrosis,” as used herein, refers to a form of traumatic cell death that results from acute cellular injury. In some embodiments, in the process of necrosis, various receptors are activated and result in the loss of cell membrane integrity and an uncontrolled release of products of cell death into the extracellular space.

In some embodiments, provided herein is a method of reducing a level of an inflammatory cytokine comprising, administering the lipoprotein system as described herein, wherein the administering is effective to reduce the level of the inflammatory cytokine. In some embodiments, the inflammatory cytokine is TNF-α or IL-6.

In some embodiments, administering the lipoprotein system as described herein reduces the level of the inflammatory cytokine in a cell, tissue or subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, at least 99.5%, at least 99.9%, or 100% as compared with an untreated control cell, tissue or subject, or compared with the corresponding activity in the same type of cell, tissue or subject before treatment with the lipoprotein system as described herein as measured by any standard technique. In some embodiments, administering the lipoprotein system as described herein reduce the level of the inflammatory cytokine in a cell, tissue or subject by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared with an untreated control cell, tissue or subject, or compared with the corresponding activity in the same type of cell, tissue or subject before treatment with the lipoprotein system as described herein as measured by any standard technique.

The term “inflammatory cytokine,” or “proinflammatory cytokine” as used herein, refers to a type of signaling molecule (a cytokine) that plays an important role in mediating the innate immune response. In some embodiments, inflammatory cytokines are secreted from immune cells like helper T cells and macrophages, and certain other cell types that promote inflammation. In some embodiments, inflammatory cytokines are predominantly produced by and involved in the upregulation of inflammatory reactions. Inflammatory cytokines include, but are not limited to, interleukin-1 (IL-1), IL-6, IL-12, IL-18, tumor necrosis factor alpha (TNF-α), interferon gamma (IFNγ), and granulocyte-macrophage colony stimulating factor (GM-CSF).

In some embodiment, administering the lipoprotein system as described herein reduces the level of IL-1, IL-6, IL-12, IL-18, TNF-α, IFNγ, GM-CSF, or any combination thereof. In some embodiment, administering the lipoprotein system as described herein reduces the level of TNF-α or IL-6. In some embodiment, administering the lipoprotein system as described herein reduces the level of TNF-α and IL-6. In some embodiment, administering the lipoprotein system as described herein reduces the level of TNF-α. In some embodiment, administering the lipoprotein system as described herein reduces the level of IL-6.

In some embodiments, administering the lipoprotein system as described herein reduces the level of the inflammatory cytokine in lung, heart, brain, microglia, blood, stomach, liver, intestine, pancreas, colon, kidney, ureter, bladder, muscle, neurons, stroma, spinal cord, ovary, vagina, prostate, bone, cartilage, ligament, or tendon.

In some embodiments, provided herein is a method of treating a condition comprising administering a therapeutically effective amount of the lipoprotein systems as described herein or the pharmaceutical composition as described herein to a subject in need thereof, wherein the providing the compositions is effective in treating the condition of the subject. In some embodiments, the compositions are administered intravenous, intranasal, intracranial, or intrathecal.

### Therapeutic Applications

The present disclosure provides a method for treating a disease, disorder and/or condition in a mammalian subject, including a human subject. Such methods may include administering to the subject the lipoprotein systems described herein or administering to the subject any of the described compositions, including pharmaceutical compositions, described herein.

In some embodiments, the disease or disorder may be a neurological disease, an autoimmune disease, a cardiovascular disease, a vascular disease, a respiratory disease, a metabolic disease, a gastrointestinal disease, a genetic syndrome, a cancer, or a multisystem disease.

In some embodiments, the present disclosure may provide a method for maintaining lipid homeostasis in subjects in need thereof, including a human subject, comprising administering the polypeptides and/or lipoprotein system of the disclosure to the subjects. In some embodiments, the polypeptides and lipoprotein systems of the disclosure may be used for facilitating lipid efflux from cells. In some embodiments, the polypeptides and lipoprotein system of the disclosure may be used for clearing lipid debris in the CNS. In some embodiments, the polypeptides and lipoprotein systems of the disclosure may be used for transporting lipids among different cells in the CNS, such as microglia, neuron, astrocytes, and oligodendrocytes. As a non-limiting example, the lipoprotein system of the disclosure may transfer lipids from neurons to astrocytes for detoxification and from astrocytes to neurons for trophic support.

In some embodiments, the present disclosure may provide a method for cell repair. As a non-limiting example, the polypeptides and lipoprotein system of the disclosure may deliver lipids to cellular membranes. As a non-limiting example, the polypeptides and lipoprotein systems of the disclosure transport myelin to oligodendrocyte progenitors for remyelination of demyelinated oligodendrocytes. The present disclosure also provides methods for tuning complement activation, for example to reduce complement activation and downstream cell inflammation and lysis.

In some embodiments, the present disclosure may provide a method for repairing BBB in subjects in need thereof, including a human subject, comprising administering a therapeutically effective amount of the polypeptides and/or the lipoprotein system of the disclosure to the subjects. In some embodiments, the polypeptides and/or lipoprotein systems of the disclosure may be used to mediate closure of the BBB after injury.

In some embodiments, provided herein is a method of treating a condition comprising administering a therapeutically effective amount of the lipoprotein system as described herein or the pharmaceutical composition as described herein to a subject in need thereof, wherein the administering is effective to treat the condition of the subject. In some embodiments, the therapeutic mechanism of action of lipoprotein system is to remove lipids from cells and tissues. In some embodiments, the lipid removed is cholesterol. In some embodiments, the lipid removed is cholesterol for the treatment of diseases marked by accumulation of cholesterol or cholesteryl esters. In some embodiments, the lipid removed is cholesterol for the treatment of diseases marked by accumulation of cholesterol or cholesteryl esters (for example, coronary artery disease or late-onset Alzheimer's disease). In some embodiments, the diseases marked by accumulation of cholesterol or cholesteryl esters is coronary artery disease or late-onset Alzheimer's disease. In some embodiments, removal of cholesterol may promote tissue repair after injury. In some embodiments, removal of cholesterol may prevent the aggregation of proteins. In some embodiments, removal of cholesterol may reduce inflammation. In some embodiments, removal of cholesterol may limit cancer cell growth or survival. In some embodiments, removal of cholesterol may limit viral entry into cells. In some embodiments, the lipid removed is oxidized or damaged. In embodiments, the lipid removed is oxidized or damaged for the treatment of diseases marked by oxidative stress or processes resulting in oxidative stress. In embodiments, the lipid removed is oxidized or damaged for the treatment of diseases marked by oxidative stress (for example, ALS or Parkinson's disease) or processes resulting in oxidative stress (for example, traumatic brain injury, ischemia/reperfusion, or chemotherapy). In embodiments, the lipid removed is oxidized or damaged for the treatment of diseases marked by oxidative stress. In embodiments, examples of the diseases marked by oxidative stress include, but are not limited to, amyotrophic lateral sclerosis (ALS) or Parkinson's disease. In embodiments, the lipid removed is oxidized or damaged for the treatment of diseases marked by processes resulting in oxidative stress. In some embodiments, diseases marked by processes resulting in oxidative stress include, but are not limited to, traumatic brain injury, ischemia/reperfusion, and chemotherapy.

In some embodiments, the lipid removed is a species that accumulates over age or due to an inborn error of metabolism. In some embodiments, the lipid removed is a species that accumulates over age. In some embodiments, the lipid removed is a species that accumulates due to an inborn error of metabolism. In some embodiments, the lipid removed is any combination of cholesterol, oxidized or damaged lipids, and an accumulated lipid species as described herein.

In some embodiments, the therapeutic mechanism of action of lipoprotein systems is to deliver payloads molecules to cells and tissues.

The interaction between neurons and neuronal cell types (e.g., glia) and the role of lipoprotein systems of the disclosure in those interactions may be studied using tri-culture organoids consisting of stem cell derived microglia, astrocytes, and neurons. Lipoprotein systems may be evaluated at basal state or in the presence of glutamic acid or amyloid beta oligomers. Individual organoids may be stained with markers associated with different cell types.

### Neurological Diseases

Various neurological diseases may be treated using a pharmaceutical composition, lipoprotein system, or polypeptide of the present disclosure. As a non-limiting example, the neurological disease may be Degenerative Cervical Myelopathy (DCM), Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Sub-cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia-Multi-Infarct, Dementia—Semantic, Dementia-Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barré Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Klüver-Bucy Syndrome, Korsakoff's Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus—Neurological Sequelae, Lyme Disease—Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myoclonic Encephalopathy of Infants, Myoclonus, Myopathy, Myopathy-Congenital, Myopathy-Thyrotoxic, Myotonia, Myotonia Congenita, Narcolepsy, Nasu Hakola disease, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy-Hereditary, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain-Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjögren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy.

In some embodiments, the neurological disease may be a neurodegenerative disease. Various neurodegenerative diseases may be treated using pharmaceutical compositions, lipoprotein system, or polypeptides of the present disclosure. As a non-limiting example, the disease may be multiple sclerosis, Parkinson's disease, Alzheimer's disease, Tay-Sachs disease, Niemann-Pick disease, Gaucher's disease, Hurler's syndrome, Huntington's disease, amyotrophic lateral sclerosis, idiopathic inflammatory demyelinating diseases, vitamin B12 deficiency, central pontine myelinolysis, tabes dorsalis, transverse myelitis, Devic's disease, progressive multifocal leukoencephalopathy, optic neuritis, traumatic injury to the CNS, an ischemic cerebral stroke, glaucoma, diabetic retinopathy, age-dependent macular degeneration, and a leukodystrophy.

Various psychological disorders may be treated with pharmaceutical compositions, lipoprotein system, or polypeptides of the present disclosure. As a non-limiting example, the psychological disorders may be Aboulia, Absence epilepsy, Acute stress Disorder, Adjustment Disorders, Adverse effects of medication NOS, Age related cognitive decline, Agoraphobia, Alcohol Addiction, Alzheimer's Disease, Amnesia (also known as Amnestic Disorder), Amphetamine Addiction, Anorexia Nervosa, Anterograde amnesia, Antisocial personality disorder (also known as Sociopathy), Anxiety Disorder (Also known as Generalized Anxiety Disorder), Anxiolytic related disorders, Asperger's Syndrome (now part of Autism Spectrum Disorder), Attention Deficit Disorder (Also known as ADD), Attention Deficit Hyperactivity Disorder (Also known as ADHD), Autism Spectrum Disorder (also known as Autism), Autophagia, Avoidant Personality Disorder, Barbiturate related disorders, Benzodiazepine related disorders, Bereavement, Bibliomania, Binge Eating Disorder, Bipolar disorder (also known as Manic Depression, includes Bipolar I and Bipolar II), Body Dysmorphic Disorder, Borderline intellectual functioning, Borderline Personality Disorder, Breathing-Related Sleep Disorder, Brief Psychotic Disorder, Bruxism, Bulimia Nervosa, Caffeine Addiction, Cannabis Addiction, Catatonic disorder, Catatonic schizophrenia, Childhood amnesia, Childhood Disintegrative Disorder (now part of Autism Spectrum Disorder), Childhood Onset Fluency Disorder (formerly known as Stuttering), Circadian Rhythm Disorders, Claustrophobia, Cocaine related disorders, Communication disorder, Conduct Disorder, Conversion Disorder, Cotard delusion, Cyclothymia (also known as Cyclothymic Disorder), Delerium, Delusional Disorder, dementia, Dependent Personality Disorder (also known as Asthenic Personality Disorder), Depersonalization disorder (now known as Depersonalization/Derealization Disorder), Depression (also known as Major Depressive Disorder), Depressive personality disorder, Derealization disorder (now known as Depersonalization/Derealization Disorder), Dermotillomania, Desynchronosis, Developmental coordination disorder, Diogenes Syndrome, Disorder of written expression, Dispareunia, Dissocial Personality Disorder, Dissociative Amnesia, Dissociative Fugue, Dissociative Identity Disorder (formerly known as Multiple Personality Disorder), Down syndrome, Dyslexia, Dyspareunia, Dysthymia (now known as Persistent Depressive Disorder), Eating disorder NOS, Ekbom's Syndrome (Delusional Parasitosis), Emotionally unstable personality disorder, Encopresis, Enuresis (bedwetting), Erotomania, Exhibitionistic Disorder, Expressive language disorder, Factitious Disorder, Female Sexual Disorders, Fetishistic Disorder, Folie n deux, Fregoli delusion, Frotteuristic Disorder, Fugue State, Ganser syndrome, Gambling Addiction, Gender Dysphoria (formerly known as Gender Identity Disorder), Generalized Anxiety Disorder, General adaptation syndrome, Grandiose delusions, Hallucinogen Addiction, Haltlose personality disorder, Histrionic Personality Disorder, Primary hypersomnia, Huntington's Disease, Hypoactive sexual desire disorder, Hypochondriasis, Hypomania, Hyperkinetic syndrome, Hypersomnia, Hysteria, Impulse control disorder, Impulse control disorder NOS, Inhalant Addiction, Insomnia, Intellectual Development Disorder, Intermittent Explosive Disorder, Joubert syndrome, Kleptomania, Korsakoff's syndrome, Lacunar amnesia, Language Disorder, Learning Disorders, Major Depression (also known as Major Depressive Disorder), major depressive disorder, Male Sexual Disorders, Malingering, Mathematics disorder, Medication-related disorder, Melancholia, Mental Retardation (now known as Intellectual Development Disorder), Misophonia, Morbid jealousy, Multiple Personality Disorder (now known as Dissociative Identity Disorder), Munchausen Syndrome, Munchausen by Proxy, Narcissistic Personality Disorder, Narcolepsy, Neglect of child, Neurocognitive Disorder (formerly known as Dementia), Neuroleptic-related disorder, Nightmare Disorder, Non Rapid Eye Movement, Obsessive-Compulsive Disorder, Obsessive-Compulsive Personality Disorder (also known as Anankastic Personality Disorder), Oneirophrenia, Onychophagia, Opioid Addiction, Oppositional Defiant Disorder, Orthorexia (ON), Pain disorder, Panic attacks, Panic Disorder, Paranoid Personality Disorder, Parkinson's Disease, Partner relational problem, Passive-aggressive personality disorder, Pathological gambling, Pedophilic Disorder, Perfectionism, Persecutory delusion, Persistent Depressive Disorder (also known as Dysthymia), Personality change due to a general medical condition, Personality disorder, Pervasive developmental disorder (PDD), Phencyclidine related disorder, Phobic disorder, Phonological disorder, Physical abuse, Pica, Polysubstance related disorder, Postpartum Depression, Post-traumatic embitterment disorder (PTED), Post Traumatic Stress Disorder, Premature ejaculation, Premenstrual Dysphoric Disorder, Psychogenic amnesia, Psychological factor affecting medical condition, Psychoneurotic personality disorder, Psychotic disorder, not otherwise specified, Pyromania, Reactive Attachment Disorder, Reading disorder, Recurrent brief depression, Relational disorder, REM Sleep Behavior Disorder, Restless Leg Syndrome, Retrograde amnesia, Retts Disorder (now part of Autism Spectrum Disorder), Rumination syndrome, Sadistic personality disorder, Schizoaffective Disorder, Schizoid Personality Disorder, Schizophrenia, Schizophreniform disorder, Schizotypal Personality Disorder, Seasonal Affective Disorder, Sedative, Hypnotic, or Anxiolytic Addiction, Selective Mutism, Self-defeating personality disorder, Separation Anxiety Disorder, Sexual Disorders Female, Sexual Disorders Male, Sexual Addiction, Sexual Masochism Disorder, Sexual Sadism Disorder, Shared Psychotic Disorder, Sleep Arousal Disorders, Sleep Paralysis, Sleep Terror Disorder (now part of Nightmare Disorder, Social Anxiety Disorder, Somatization Disorder, Specific Phobias, Stendhal syndrome, Stereotypic movement disorder, Stimulant Addiction, Stuttering (now known as Childhood Onset Fluency Disorder), Substance related disorder, Tardive dyskinesia, Tobacco Addiction, Tourettes Syndrome, Transient tic disorder, Transient global amnesia, Transvestic Disorder, Trichotillomania, Undifferentiated Somatoform Disorder, Vaginismus, and Voyeuristic Disorder.

In some embodiments, the lipoprotein systems of the disclosure may be used to treat or prevent Niemann Pick disease. Niemann-Pick disease is a genetic disorder affecting the body's ability to metabolize cholesterol and lipids, causing the loss of function of nerves, the brain, and various organs. Niemann-Pick disease is divided based on the genetic cause. Type A and B are caused by a mutation in the SMPD1 gene, leading to a shortage of the enzyme acid sphingomyelin phosphodiesterase 1, which is necessary for the breakdown of fats. As a result, sphingomyelin accumulates in the lysosomes, disrupting normal cell function. Types C1 and C2 are caused by mutations in the NPC1 or NPC2 genes. The enzymes produced from these genes, NPC intracellular cholesterol transporter 1 and NPC intracellular cholesterol transporter 2, are involved in the movement of lipids within cells. The absence or reduced activity of these enzymes leads to the toxic accumulation of cholesterol and other lipids in cells. The lipoprotein system and methods of using the lipoprotein systems described in the present disclosure may be used to prevent, manage and/or treat NPC1. The compositions of the disclosure may be able to clear the accumulations of cholesterol, sphingomyelin, and other lipids in cells. In some embodiments, the lipoprotein system can also be used as a carrier to deliver gene therapies to treat NPC.

In some embodiments, the compositions and methods of the present disclosure may be used to treat subjects suffering from Alzheimer's Disease (AD). In some cases, methods of the present disclosure may be used to treat subjects suspected of developing AD. Alzheimer's Disease (AD) is a debilitating neurodegenerative disease and the most common form of dementia affecting the memory, thinking and behavior. Typical early symptom is difficulty of remembering newly learned information. As the disease advances, symptoms include disorientation, changes in sleep, changes in mood and behavior, confusion, unfound suspicions and eventually difficulty speaking, swallowing, and walking. The AD brain is characterized by dual aggregates, the extracellular β-amyloid plaques, and the intracellular neurofibrillary tangles (NFT) of misfolded, hyperphosphorylated microtubule associated, tau proteins. The β-amyloid plaques may lead to pathological cascades that are associated with a number of proteins, such as, but not limited to, APP (amyloid beta (A4) precursor protein), A beta (amyloid beta), BACE (Beta-secretases), and APOE (apolipoprotein E). Apolipoprotein E (APOE) is the major cholesterol carrier in the brain, affecting various normal cellular processes including neuronal growth, repair and remodeling of membranes, synaptogenesis, clearance, and degradation of amyloid (Ab) and neuroinflammation. Among the three common APOE variants (E2, E3, and E4), APOE4 is considered the strongest genetic risk factor for Alzheimer's disease (AD), whereas APOE2 is neuroprotective. The potentials to improve the APOE lipidation for the treatment of AD and other APOE-associated central nervous system impairments has been examined by Lanfranco et al. (Int. J. Mol. Sci. 2020, 21, 6336; the contents are incorporated by references in its entirety). The polypeptides, and lipoprotein system and methods of using the polypeptides and lipoprotein systems described in the present disclosure may be used to prevent, manage and/or treat AD. In some embodiments, the polypeptides, a lipoprotein system, and methods of using the polypeptides and lipoprotein systems described in the present disclosure may be used to prevent, manage and/or treat AD for APOE4 carriers.

The lipoprotein system and methods of using the lipoprotein systems described in the present disclosure may be used to prevent, manage and/or treat Parkinson's Disease (PD). In some embodiments, the polypeptides and/or lipoprotein system of this disclosure may be used to prevent, manage and/or treat PD by repairing CNS cells. In some embodiments, the polypeptides and/or lipoprotein system of this disclosure may be used to prevent, manage and/or treat PD in subjects in need who are APOE4 carriers. Parkinson's Disease (PD) is a progressive disorder of the nervous system affecting especially the substantia nigra of the brain. PD develops are a result of the loss of dopamine producing brain cells. Typical early symptoms of PD include shaking or trembling of a limb, e.g., hands, arms, legs, feet, and face. Additional characteristic symptoms are stiffness of the limbs and torso, slow movement, or an inability to move, impaired balance and coordination, cognitional changes, and psychiatric conditions e.g., depression and visual hallucinations. PD is related to other diseases related to alpha-synuclein aggregation, referred to as “synucleinopathies,” which may be treated using the compositions of the disclosure. Such diseases include, but are not limited to, Parkinson's Disease Dementia (PDD), multiple system atrophy (MSA), dementia with Lewy bodies, juvenile-onset generalized neuroaxonal dystrophy (Hallervorden-Spatz disease), pure autonomic failure (PAF), neurodegeneration with brain iron accumulation type-1 (NBIA-1) and combined Alzheimer's and Parkinson's disease. In some embodiments, the payloads of the disclosure may be carbidopa and levodopa combination for reducing stiffness and slow movement, and anticholinergics to reduce trembling and stiffness. In other aspects, the payloads of the disclosure may be antibodies targeting alpha-synuclein protein, or other proteins relevant for brain cell death in PD, may be used to prevent and/or treat PD.

The lipoprotein systems and methods of using the lipoprotein systems described in the present disclosure may be used to prevent, manage and/or treat Gaucher disease (GD). In some embodiments, the polypeptides and/or lipoprotein systems of the disclosure may be used to prevent, manage and/or treat GD by clearing lipid debris, repairing cells, or restoring lipid homeostasis. In some embodiments, the payloads of the disclosure may be recombinant enzymes, imiglucerase, taliglucerase alfa, and velaglucerase alfa that are useful in the treatment of GD. Deficiency of Glucocerebrosidase (GCase) is the underlying mechanism of GD. Low GCase activity leads to accumulation of glucocerebroside and other glycolipids within the lysosomes of macrophages. Accumulation can amount to about 20-fold to about 100-fold higher than in control cells or subjects without GCase deficiency. Pathologic lipid accumulation in macrophages accounts for <2% of additional tissue mass observed in the liver and spleen of GD patients. Additional increase in organ weight and volume is attributed to an inflammatory and hyperplastic cellular response.

Dementia with Lewy Bodies (DLB), also known as diffuse Lewy body disease, is a form of progressive dementia, characterized by cognitive decline, fluctuating alertness and attention, visual hallucinations and parkinsonian motor symptoms. DLB is caused by the abnormal build-up of Lewy bodies, aggregates of the alpha-synuclein protein, in the cytoplasm of neurons in the brain areas controlling memory and motor control. The pathophysiology of these aggregates is similar to aggregates observed in Parkinson's disease and DLB also has similarities to Alzheimer's disease. Inherited DLB has been associated with gene mutations in SNCA and SNCB genes, producing synuclein proteins. The lipoprotein system and methods of using the lipoprotein systems described in the present disclosure may be used to prevent, manage and/or treat DLB. In some embodiments, the polypeptides and/or lipoprotein system of this disclosure may be used to prevent, manage and/or treat DLB by repairing CNS cells.

Traumatic damage to the CNS is characterized by a physical impact on the central nervous system. For example, traumatic injury of the brain occurs when the brain is subjected to physical forces that result in progressive neuronal damage and/or cell death. Pericytes, embedded in the basement membrane of blood capillaries and regulating BBB permeability may detach from the neurovasculature following TBI. Pericytes secrete APOE to mediate closure of the leaky BBB after injury. Clinical studies of traumatic brain injury (TBI) have shown that possession of the APOE4 allele is associated with unfavorable outcomes (Teasdale et al. Lancet 19973501069-1071). The lipoprotein system lipoprotein system and methods of using the lipoprotein systems described in the present disclosure may be used to prevent, manage and/or treat traumatic CNS injury such as TBI, including blunt trauma as well as laceration trauma.

Chronic traumatic encephalopathy (CTE) is a neurodegenerative disease marked by widespread accumulation of hyperphosphorylated tau (p-tau). Stern et al. examined APOE genotypes of 36 athletes that were neuropathologically confirmed CTE without comorbid neurodegenerative or motor neuron disease and found the proportions of APOE4 genotypes (homozygotes and heterozygotes) in the CTE patients were significantly higher compared to the healthy control (Stern et al. Neurol. 2013, 81 (13), 1122-1129). The lipoprotein system and methods of using the lipoprotein systems described in the present disclosure may be used to prevent, manage and/or treat CTE. In some embodiments, the polypeptides and/or lipoprotein system of this disclosure may be used to prevent, manage and/or treat CTE in subjects in need who have an APOE4 genotype.

In some embodiments, the polypeptides of the disclosure may be used to treat, prevent, and/or treat stroke, Degenerative Cervical Myelopathy, Post-Operative Cognitive Dysfunction (POCD) and/or cancer related cognitive impairment (CRCI).

### Metabolic Diseases

In some embodiments, the compositions of the disclosure and/or lipoprotein systems of the disclosure may be used to treat metabolic diseases. In some embodiments, the compositions of the disclosure and/or lipoprotein systems of the disclosure may be used to treat Smith Lemli Opitz Syndrome (or SLOS, or 7-dehydrocholesterol reductase deficiency). SLOS is an inborn error of cholesterol synthesis. In addition to microcephaly, moderate to severe intellectual disability, sensory hypersensitivity, stereotyped behaviors, dysmorphic facial features, and syndactyly of the second/third toes, a feature of the disease is reduced cerebrosterol (24(S)-hydroxycholesterol) levels. SLOS is an autosomal recessive genetic condition resulting from deficiency in the final enzyme of the cholesterol synthesis pathway and causes low or low-normal plasma cholesterol levels and increased 7- and 8-dehydrocholesterol (DHC; 7DHC and 8DHC) levels. Witsch-Baumgartner et al. reported that the maternal apolipoprotein E genotype is a modifier of the clinical severity of SLOS. Specifically, maternal APOE2 was associated with a more severe phenotype (Witsch-Baumgartner et al. J Med Genet 2004; 41:577-584; the contents of which are herein incorporated by reference in its entirety). In addition, A SLOS mouse model study showed that lack of functional apolipoprotein E exacerbates the neonatal lethality of SLOS mouse (Solcà et al. Molecular Genetics and Metabolism; Volume 91, Issue 1, May 2007, Pages 7-14; the contents of which are herein incorporated by reference in its entirety). Together, these studies suggest that lipoprotein systems of the disclosure containing apolipoprotein polypeptides and/or lipoprotein systems including cholesterol, and/or agents that modulate cholesterol production e.g., may be useful in the treatment of SLOS.

### Respiratory Diseases

In some embodiments, the compositions of the disclosure and/or lipoprotein systems of the disclosure may be used to treat respiratory diseases.

In some embodiments, the compositions, and/or lipoprotein systems of the disclosure may be used to treat COVID-19 or a severe acute respiratory syndrome coronavirus 2 (SARS CoV2) infection. In some embodiments, the compositions, and/or lipoprotein systems of the disclosure may be used to treat neurological symptoms associated with COVID-19 or a SARS CoV2 infection. The new coronavirus of 2019, named SARS CoV2, causes a severe respiratory disease referred to as COVID-19 which has spread rapidly on a global scale. SARS-CoV-2 is the most serious coronavirus outbreak in history. There is an urgent need for therapeutics, prophylactics, and diagnostics to treat, prevent, and detect SARS-CoV-2 infection and COVID-19.

Apolipoprotein E4 allele (APOE4) has been reported to associate with increased susceptibility to SARS CoV-2 infection and COVID-19 mortality in several genetic studies (see Hubacek et al., Gerontology 2021; 67:320-322; AI-Jaf et al., Infect Genet Evol. 2021; 95:105043; Teodoro et al. Gerontology 2021; 67:281-289; the contents of each of which are herein incorporated by reference in its entirety. For example, Kurki et al. studied the genetic association between APOE and COVID-19 in Finnish biobank, autopsy and prospective clinical cohort datasets (Kurki et al. Acta Neuropathologica Communications (2021) 9:199; the contents are incorporated by reference in its entirety). Their data showed that APOE4 allele associated with severe COVID-19 in intensive care patients compared with non-infected population controls after matching for age, sex and cardiovascular disease status. Histopathological examination of brain autopsy material of COVID-19 cases provided evidence that perivascular microhaemorrhages are more prevalent in APOE4 allele carrying individuals. In addition, their analysis of post-COVID fatigue revealed that the presence of APOE4 allele independently associates with higher mental fatigue compared to non-carriers at six months after initial illness. This study indicates that APOE4 is a risk factor for severe COVID-19 and post COVID mental fatigue. In some embodiments, compositions and/or lipoprotein systems of the disclosure may be used to treat post COVID mental fatigue.

### Autoimmune Diseases

Various autoimmune diseases and autoimmune-related diseases may be treated with pharmaceutical compositions, lipoprotein system, or polypeptides of the present disclosure. As used herein, the term “autoimmune disease” refers to a disease in which the body produces antibodies that attack its own tissues. As a non-limiting example, the autoimmune disease may be Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal & neuronal neuropathies, Balo disease, Behcet's disease, Bullous pemphigoid, Cardiomyopathy, Castleman disease, Celiac disease, Chagas disease, Chronic fatigue syndrome**, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia**, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosis, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Ulcerative colitis, Undifferentiated connective tissue disease (UCTD), Uveitis, Vasculitis, Vesiculobullous dermatosis, Vitiligo, and Wegener's granulomatosis (now termed Granulomatosis with Polyangiitis (GPA).

The isoform-specific effects of APOE on Guillain-Barr6 syndrome (GBS) have been studied and it was found that the most severe clinical course occurring on APOE ε4 transgenic mice. The lipoprotein system and methods of using the lipoprotein systems described in the present disclosure may be used to prevent, manage and/or treat GBS. In some embodiments, the polypeptides and/or lipoprotein system of the disclosure may be used to prevent, manage and/or treat GBS in subjects in need who are APOE4 carriers.

### Cardiovascular Diseases

Various cardiovascular diseases may be treated with pharmaceutical compositions, lipoprotein system, or polypeptides of the present disclosure. As a non-limiting example, the cardiovascular disease may be ischemic heart disease also known as coronary artery disease, cerebrovascular disease (Stroke), Peripheral vascular disease, Heart failure, Rheumatic heart disease, and Congenital heart disease.

Various blood diseases may be treated with pharmaceutical compositions, lipoprotein system, or polypeptides of the present disclosure. As a non-limiting example, the blood diseases may be Anemia and CKD (for health care professionals), Aplastic Anemia and Myelodysplastic Syndromes, Deep Vein Thrombosis, Hemochromatosis, Hemophilia, Henoch-Schonlein Purpura, Idiopathic Thrombocytopenic Purpura, Iron-Deficiency Anemia, Pernicious Anemia, Pulmonary Embolism, Sickle Cell Anemia, Sickle Cell Trait and Other Hemoglobinopathies, Thalassemia, Thrombotic Thrombocytopenic Purpura, and Von Willebrand Disease.

The compositions described in the present disclosure may be used to prevent, manage and/or treat Dilated Cardiomyopathy. In some embodiments, the polypeptides and/or lipoprotein systems of the disclosure may be used to prevent, manage and/or treat Dilated Cardiomyopathy in subject. Dilated Cardiomyopathy may be associated with APOE genotypes. APOE4 carriers have increased risk of developing Dilated Cardiomyopathy. Jurkovicova et al. reported higher frequency of APOE4 alleles in patients with Dilated Cardiomyopathy compared to control suggesting a link with Dilated Cardiomyopathy (Jurkovicova et al. Gen Physiol Biophys 2006 March; 25(1): 3-10).

### Genetic Syndromes

Various genetic syndromes or rare diseases may be treated with pharmaceutical compositions, lipoprotein system, or polypeptides of the present disclosure. As used herein, the term “rare disease” refers to any disease that affects a small percentage of the population. As a non-limiting example, the rare disease may be Acrocephalosyndactylia, Acrodermatitis, Addison Disease, Adie Syndrome, Alagille Syndrome, Amylose, Amyotrophic Lateral Sclerosis, Angelman Syndrome, Angiolymphoid Hyperplasia with Eosinophilia, Arnold-Chiari Malformation, Arthritis, Juvenile Rheumatoid, Asperger Syndrome, Bardet-Biedl Syndrome, Barrett Esophagus, Beckwith-Wiedemann Syndrome, Behcet Syndrome, Bloom Syndrome, Bowen's Disease, Brachial Plexus Neuropathies, Brown-Sequard Syndrome, Budd-Chiari Syndrome, Burkitt Lymphoma, Carcinoma 256, Walker, Caroli Disease, Charcot-Marie-Tooth Disease, Chediak-Higashi Syndrome, Chiari-Frommel Syndrome, Chondrodysplasia Punctata, Colonic Pseudo-Obstruction, Colorectal Neoplasms, Hereditary Nonpolyposis, Craniofacial Dysostosis, Creutzfeldt-Jakob Syndrome, Crohn Disease, Cushing Syndrome, Cystic Fibrosis, Dandy-Walker Syndrome, De Lange Syndrome, Dementia, Vascular, Dermatitis Herpetiformis, DiGeorge Syndrome, Diffuse Cerebral Sclerosis of Schilder, Duane Retraction Syndrome, Dupuytren Contracture, Ebstein Anomaly, Eisenmenger Complex, Ellis-Van Creveld Syndrome, Encephalitis, Enchondromatosis, Epidermal Necrolysis, Toxic, Facial Hemiatrophy, Factor XII Deficiency, Fanconi Anemia, Felty's Syndrome, Fibrous Dysplasia, Polyostotic, Fox-Fordyce Disease, Friedreich Ataxia, Fusobacterium, Gardner Syndrome, Gaucher Disease, Gerstmann Syndrome, Giant Lymph Node Hyperplasia, Glycogen Storage Disease Type I, Glycogen Storage Disease Type II, Glycogen Storage Disease Type IV, Glycogen Storage Disease Type V, Glycogen Storage Disease Type VII, Goldenhar Syndrome, Guillain-Barre Syndrome, Hallermann's Syndrome, Hamartoma Syndrome, Multiple, Hartnup Disease, Hepatolenticular Degeneration, Hepatolenticular Degeneration, Hereditary Sensory and Motor Neuropathy, Hirschsprung Disease, Histiocytic Necrotizing Lymphadenitis, Histiocytosis, Langerhans-Cell, Hodgkin Disease, Horner Syndrome, Huntington Disease, Hyperaldosteronism, Hyperhidrosis, Hyperostosis, Diffuse Idiopathic Skeletal, Hypopituitarism, Inappropriate ADH Syndrome, Intestinal Polyps, Isaacs Syndrome, Kartagener Syndrome, Kearns-Sayre Syndrome, Klippel-Feil Syndrome, Klippel-Trenaunay-Weber Syndrome, Kluver-Bucy Syndrome, Korsakoff Syndrome, Lafora Disease, Lambert-Eaton Myasthenic Syndrome, Landau-Kleffner Syndrome, Langer-Giedion Syndrome, Leigh Disease, Lesch-Nyhan Syndrome, Leukodystrophy, Globoid Cell, Li-Fraumeni Syndrome, Long QT Syndrome, Machado-Joseph Disease, Mallory-Weiss Syndrome, Marek Disease, Marfan Syndrome, Meckel Diverticulum, Meige Syndrome, Melkersson-Rosenthal Syndrome, Meniere Disease, Mikulicz' Disease, Miller Fisher Syndrome, Mobius Syndrome, Moyamoya Disease, Mucocutaneous Lymph Node Syndrome, Mucopolysaccharidosis I, Mucopolysaccharidosis II, Mucopolysaccharidosis III, Mucopolysaccharidosis IV, Mucopolysaccharidosis VI, Multiple Endocrine Neoplasia Type 1, Munchausen Syndrome by Proxy, Muscular Atrophy, Spinal, Narcolepsy, Neuroaxonal Dystrophies, Neuromyelitis Optica, Neuronal Ceroid-Lipofuscinoses, Niemann-Pick Diseases, Noonan Syndrome, Optic Atrophies, Hereditary, Osteitis Deformans, Osteochondritis, Osteochondrodysplasias, Osteolysis, Essential, Paget Disease Extramammary, Paget's Disease, Mammary, Panniculitis, Nodular Nonsuppurative, Papillon-Lefevre Disease, Paralysis, Pelizaeus-Merzbacher Disease, Pemphigus, Benign Familial, Penile Induration, Pericarditis, Constrictive, Peroxisomal Disorders, Peutz-Jeghers Syndrome, Pick Disease of the Brain, Pierre Robin Syndrome, Pigmentation Disorders, Pityriasis Lichenoides, Polycystic Ovary Syndrome, Polyendocrinopathies, Autoimmune, Prader-Willi Syndrome, Pupil Disorders, Rett Syndrome, Reye Syndrome, Rubinstein-Taybi Syndrome, Sandhoff Disease, Sarcoma, Ewing's, Schnitzler Syndrome, Sjogren's Syndrome, Sjogren-Larsson Syndrome, Smith-Lemli-Opitz Syndrome, Spinal Muscular Atrophies of Childhood, Sturge-Weber Syndrome, Gustatory, Takayasu Arteritis, Tangier Disease, Tay-Sachs Disease, Thromboangiitis Obliterans, Thyroiditis, Autoimmune, Tietze's Syndrome, Togaviridae Infections, Tolosa-Hunt Syndrome, Tourette Syndrome, Uveomeningoencephalitic Syndrome, Waardenburg's Syndrome, Wegener Granulomatosis, Weil Disease, Werner Syndrome, Williams Syndrome, Wilms Tumor, Wolff-Parkinson-White Syndrome, Wolfram Syndrome, Wolman Disease, Zellweger Syndrome, Zollinger-Ellison Syndrome, and von Willebrand Diseases.

### Cancer

Various cancers may be treated with pharmaceutical compositions, lipoprotein system, or polypeptides of the present disclosure. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

Types of carcinomas which may be treated with pharmaceutical compositions, lipoprotein system, or polypeptides of the present disclosure include, but are not limited to, papilloma/carcinoma, choriocarcinoma, endodermal sinus tumor, teratoma, adenoma/adenocarcinoma, melanoma, hepatocellular carcinoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma.

Types of sarcomas which may be treated with pharmaceutical compositions, lipoprotein system, or polypeptides of the present disclosure include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

As a non-limiting example, the cancer which may be treated may be Acute granulocytic leukemia, Acute lymphocytic leukemia, Acute myelogenous leukemia, Adenocarcinoma, Adenosarcoma, Adrenal cancer, Adrenocortical carcinoma, Anal cancer, Anaplastic astrocytoma, Angiosarcoma, Appendix cancer, Astrocytoma, Basal cell carcinoma, B-Cell lymphoma), Bile duct cancer, Bladder cancer, Bone cancer, Bowel cancer, Brain cancer, Brain stem glioma, Brain tumor, Breast cancer, Carcinoid tumors, Cervical cancer, Cholangiocarcinoma, Chondrosarcoma, Chronic lymphocytic leukemia, Chronic myelogenous leukemia, Colon cancer, Colorectal cancer, Craniopharyngioma, Cutaneous lymphoma, Cutaneous melanoma, Diffuse astrocytoma, Ductal carcinoma in situ, Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors, General, Germ cell tumor, Glioblastoma multiforme, Glioma, Hairy cell leukemia, Head and neck cancer, Hemangioendothelioma, Hodgkin lymphoma, Hodgkin's disease, Hodgkin's lymphoma, Hypopharyngeal cancer, Infiltrating ductal carcinoma, Infiltrating lobular carcinoma, Inflammatory breast cancer, Intestinal Cancer, Intrahepatic bile duct cancer, Invasive/infiltrating breast cancer, Islet cell cancer, Jaw cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Nasal cavity cancer, Nasopharyngeal cancer, Neck cancer, Neuroblastoma, Neuroendocrine tumors, Non-Hodgkin lymphoma, Non-Hodgkin's lymphoma, Non-small cell lung cancer, Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Paget's disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pineoblastoma, Pituitary gland cancer, Primary central nervous system lymphoma, Prostate cancer, Rectal cancer, Renal cell cancer, Renal pelvis cancer, Rhabdomyosarcoma, Salivary gland cancer, Sarcoma, Sarcoma, bone, Sarcoma, soft tissue, Sarcoma, uterine, Sinus cancer, Skin cancer, Small cell lung cancer, Small intestine cancer, Soft tissue sarcoma, Spinal cancer, Spinal column cancer, Spinal cord cancer, Spinal tumor, Squamous cell carcinoma, Stomach cancer, Synovial sarcoma, T-cell lymphoma), Testicular cancer, Throat cancer, Thymoma/thymic carcinoma, Thyroid cancer, Tongue cancer, Tonsil cancer, Transitional cell cancer, Transitional cell cancer, Transitional cell cancer, Triple-negative breast cancer, Tubal cancer, Tubular carcinoma, Ureteral cancer, Ureteral cancer, Urethral cancer, Uterine adenocarcinoma, Uterine cancer, Uterine sarcoma, Vaginal cancer, and Vulvar cancer.

### Infectious Disease

Various infectious diseases may be treated with pharmaceutical compositions, lipoprotein system, or polypeptides of the present disclosure. As used herein, the term “infectious disease” refers to any disorders caused by organisms such as bacteria, viruses, fungi or parasites. As a non-limiting example, the infectious disease may be Acute bacterial rhinosinusitis, 14-day measles, Acne, Acrodermatitis chronica atrophicans (ACA)-(late skin manifestation of latent Lyme disease), Acute hemorrhagic conjunctivitis, Acute hemorrhagic cystitis, Acute rhinosinusitis, Adult T-cell Leukemia-Lymphoma (ATLL), African Sleeping Sickness, AIDS (Acquired Immunodeficiency Syndrome), Alveolar hydatid, Amebiasis, Amebic meningoencephalitis, Anaplasmosis, Anthrax, Arboviral or parainfectious, Ascariasis (Roundworm infections), Aseptic meningitis, Athlete's foot (Tinea pedis), Australian tick typhus, Avian Influenza, Babesiosis, Bacillary angiomatosis, Bacterial meningitis, Bacterial vaginosis, Balanitis, Balantidiasis, Bang's disease, Barmah Forest virus infection, Bartonellosis (Verruga peruana; Carrion's disease; Oroya fever), Bat Lyssavirus Infection, Bay sore (Chiclero's ulcer), Baylisascaris infection (Racoon roundworm infection), Beaver fever, Beef tapeworm, Bejel (endemic syphilis), Biphasic meningoencephalitis, Black Bane, Black death, Black piedra, Blackwater Fever, Blastomycosis, Blennorrhea of the newborn, Blepharitis, Boils, Bornholm disease (pleurodynia), Borrelia miyamotoi Disease, Botulism, Boutonneuse fever, Brazilian purpuric fever, Break Bone fever, Brill, Bronchiolitis, Bronchitis, Brucellosis (Bang's disease), Bubonic plague, Bullous impetigo, Burkholderia mallei (Glanders), Burkholderia pseudomallei (Melioidosis), Buruli ulcers (also Mycoburuli ulcers), Busse, Busse-Buschke disease (Cryptococcosis), California group encephalitis, Campylobacteriosis, Candidiasis, Canefield fever (Canicola fever; 7-day fever; Weil's disease; leptospirosis; canefield fever), Canicola fever, Capillariasis, Carate, Carbapenem-resistant Enterobacteriaceae (CRE), Carbuncle, Carrion's disease, Cat Scratch fever, Cave disease, Central Asian hemorrhagic fever, Central European tick, Cervical cancer, Chagas disease, Chancroid (Soft chancre), Chicago disease, Chickenpox (Varicella), Chiclero's ulcer, Chikungunya fever, Chlamydial infection, Cholera, Chromoblastomycosis, Ciguatera, Clap, Clonorchiasis (Liver fluke infection), Clostridium Difficile Infection, ClostriDium Perfringens (Epsilon Toxin), Coronavirus disease, COVID19, Coccidioidomycosis fungal infection (Valley fever; desert rheumatism), Coenurosis, Colorado tick fever, Condyloma accuminata, Condyloma accuminata(Warts), Condyloma lata, Congo fever, Congo hemorrhagic fever virus, Conjunctivitis, cowpox, Crabs, Crimean, Croup, Cryptococcosis, Cryptosporidiosis (Crypto), Cutaneous Larval Migrans, Cyclosporiasis, Cystic hydatid, Cysticercosis, Cystitis, Czechoslovak tick, D68 (EV-D68), Dacryocytitis, Dandy fever, Darling's Disease, Deer fly fever, Dengue fever (1, 2, 3 and 4), Desert rheumatism, Devil's grip, Diphasic milk fever, Diphtheria, Disseminated Intravascular Coagulation, Dog tapeworm, Donovanosis, Donovanosis (Granuloma inguinale), Dracontiasis, Dracunculosis, Duke's disease, Dum Dum Disease, Durand-Nicholas-Favre disease, Dwarf tapeworm, E. coli infection (E. coli), Eastern equine encephalitis, Ebola Hemorrhagic Fever (Ebola virus disease EVD), Ectothrix, Ehrlichiosis (Sennetsu fever), Encephalitis, Endemic Relapsing fever, Endemic syphilis, Endophthalmitis, Endothrix, Enterobiasis (Pinworm infection), Enterotoxin—B Poisoning (Staph Food Poisoning), Enterovirus Infection, Epidemic Keratoconjunctivitis, Epidemic Relapsing fever, Epidemic typhus, Epiglottitis, Erysipelis, Erysipeloid (Erysipelothricosis), Erythema chronicum migrans, Erythema infectiosum, Erythema marginatum, Erythema multiforme, Erythema nodosum, Erythema nodosum leprosum, Erythrasma, Espundia, Eumycotic mycetoma, European blastomycosis, Exanthem subitum (Sixth disease), Eyeworm, Far Eastern tick, Fascioliasis, Fievre boutonneuse (Tick typhus), Fifth Disease (erythema infectiosum), Filatow-Dukes' Disease (Scalded Skin Syndrome; Ritter's Disease), Fish tapeworm, Fitz-Hugh-Curtis syndrome—Perihepatitis, Flinders Island Spotted Fever, Flu (Influenza), Folliculitis, Four Corners Disease, Four Corners Disease (Human Pulmonary Syndrome (HPS)), Frambesia, Francis disease, Furunculosis, Gas gangrene, Gastroenteritis, Genital Herpes, Genital Warts, German measles, Gerstmann-Straussler-Scheinker (GSS), Giardiasis, Gilchrist's disease, Gingivitis, Gingivostomatitis, Glanders, Glandular fever (infectious mononucleosis), Gnathostomiasis, Gonococcal Infection (Gonorrhea), Gonorrhea, Granuloma inguinale (Donovanosis), Guinea Worm, Haemophilus Influenza disease, Hamburger disease, Hansen's disease—leprosy, Hantaan disease, Hantaan-Korean hemorrhagic fever, Hantavirus Pulmonary Syndrome, Hantavirus Pulmonary Syndrome (HPS), Hard chancre, Hard measles, Haverhill fever—Rat bite fever, Head and Body Lice, Heartland fever, Helicobacterosis, Hemolytic Uremic Syndrome (HUS), Hepatitis A, Hepatitis B, Hepatitis C, Hepatitis D, Hepatitis E, Herpangina, Herpes-genital, Herpes labialis, Herpes-neonatal, Hidradenitis, Histoplasmosis, Histoplasmosis infection (Histoplasmosis), His-Werner disease, HIV infection, Hookworm infections, Hordeola, Hordeola (Stye), HTLV, HTLV-associated myelopathy (HAM), Human granulocytic ehrlichiosis, Human monocytic ehrlichiosis, Human Papillomavirus (HPV), Human Pulmonary Syndrome, Hydatid cyst, Hydrophobia, Impetigo, Including congenital (German Measles), Inclusion conjunctivitis, Inclusion conjunctivitis—Swimming Pool conjunctivitis-Pannus, Infantile diarrhea, Infectious Mononucleosis, Infectious myocarditis, Infectious pericarditis, Influenza, Isosporiasis, Israeli spotted fever, Japanese Encephalitis, Jock itch, Jorge Lobo disease—lobomycosis, Jungle yellow fever, Junin Argentinian hemorrhagic fever, Kala Azar, Kaposi's sarcoma, Keloidal blastomycosis, Keratoconjunctivitis, Kuru, Kyasanur forest disease, LaCrosse encephalitis, Lassa hemorrhagic fever, Legionellosis (Legionnaires Disease), Legionnaire's pneumonia, Lemierre's Syndrome (Postanginal septicemia), Lemming fever, Leprosy, Leptospirosis (Nanukayami fever; Weil's disease), Listeriosis (Listeria), Liver fluke infection, Lobo's mycosis, Lockjaw, Loiasis, Louping III, Ludwig's angina, Lung fluke infection, Lung fluke infection (Paragonimiasis), Lyme disease, Lymphogranuloma venereum infection (LGV), Machupo Bolivian hemorrhagic fever, Madura foot, Mal del pinto, Malaria, Malignant pustule, Malta fever, Marburg hemorrhagic fever, Masters disease, Maternal Sepsis (Puerperal fever), Measles, Mediterranean spotted fever, Melioidosis (Whitmore's disease), Meningitis, Meningococcal Disease, MERS, Milker's nodule, Molluscum contagiosum, Moniliasis, monkeypox, Mononucleosis, Mononucleosis-like syndrome, Montezuma's Revenge, Morbilli, MRSA (methicillin-resistant Staphylococcus aureus) infection, Mucormycosis-Zygomycosis, Multiple Organ Dysfunction Syndrome or MODS, Multiple-system atrophy (MSA), Mumps, Murine typhus, Murray Valley Encephalitis (MVE), Mycoburuli ulcers, Mycoburuli ulcers-Buruli ulcers, Mycotic vulvovaginitis, Myositis, Nanukayami fever, Necrotizing fasciitis, Necrotizing fasciitis-Type 1, Necrotizing fasciitis-Type 2, Negishi, New world spotted fever, Nocardiosis, Nongonococcal urethritis, Non-Polio (Non-Polio Enterovirus), Norovirus infection, North American blastomycosis, North Asian tick typhus, Norwalk virus infection, Norwegian itch, O'Hara disease, Omsk hemorrhagic fever, Onchoceriasis, Onychomycosis, Opisthorchiasis, Opthalmia neonatorium, Oral hairy leukoplakia, Orf, Oriental Sore, Oriental Spotted Fever, Ornithosis (Parrot fever; Psittacosis), Oroya fever, Otitis externa, Otitis media, Pannus, Paracoccidioidomycosis, Paragonimiasis, Paralytic Shellfish Poisoning (Paralytic Shellfish Poisoning), Paronychia (Whitlow), Parotitis, PCP pneumonia, Pediculosis, Peliosis hepatica, Pelvic Inflammatory Disease, Pertussis (also called Whooping cough), Phaeohyphomycosis, Pharyngoconjunctival fever, Piedra (White Piedra), Piedra (Black Piedra), Pigbel, Pink eye conjunctivitis, Pinta, Pinworm infection, Pitted Keratolysis, Pityriasis versicolor (Tinea versicolor), Plague; Bubonic, Pleurodynia, Pneumococcal Disease, Pneumocystosis, Pneumonia, Pneumonic (Plague), Polio or Poliomyelitis, Polycystic hydatid, Pontiac fever, Pork tapeworm, Posada-Wernicke disease, Postanginal septicemia, Powassan, Progressive multifocal leukencephalopathy, Progressive Rubella Panencephalitis, Prostatitis, Pseudomembranous colitis, Psittacosis, Puerperal fever, Pustular Rash diseases (Small pox), Pyelonephritis, Pylephlebitis, Q-Fever, Quinsy, Quintana fever (5-day fever), Rabbit fever, Rabies, Racoon roundworm infection, Rat bite fever, Rat tapeworm, Reiter Syndrome, Relapsing fever, Respiratory syncytial virus (RSV) infection, Rheumatic fever, Rhodotorulosis, Ricin Poisoning, Rickettsialpox, Rickettsiosis, Rift Valley Fever, Ringworm, Ritter's Disease, River Blindness, Rocky Mountain spotted fever, Rose Handler's disease (Sporotrichosis), Rose rash of infants, Roseola, Ross River fever, Rotavirus infection, Roundworm infections, Rubella, Rubeola, Russian spring, Salmonellosis gastroenteritis, San Joaquin Valley fever, Sao Paulo Encephalitis, Sao Paulo fever, SARS, Scabies Infestation (Scabies) (Norwegian itch), Scalded Skin Syndrome, Scarlet fever (Scarlatina), Schistosomiasis, Scombroid, Scrub typhus, Sennetsu fever, Sepsis (Septic shock), Severe Acute Respiratory Syndrome (SARS), SARS-CoV2 infection, Shiga Toxigenic Escherichia coli (STEC/VTEC), Shigellosis gastroenteritis (Shigella), Shinbone fever, Shingles, Shipping fever, Siberian tick typhus, Sinusitis, Sixth disease, Slapped cheek disease, Sleeping sickness, Smallpox (Variola), Snail Fever, Soft chancre, Southern tick associated rash illness, Sparganosis, Spelunker's disease, Sporadic typhus, Sporotrichosis, Spotted fever, Spring, St. Louis encephalitis, Staphylococcal Food Poisoning, Staphylococcal Infection, Strep. throat, Streptococcal Disease, Streptococcal Toxic-Shock Syndrome, Strongyloiciasis, Stye, Subacute Sclerosing Panencephalitis, Subacute Sclerosing Panencephalitis (SSPE), Sudden Acute Respiratory Syndrome, Sudden Rash, Swimmer's ear, Swimmer's Itch, Swimming Pool conjunctivitis, Sylvatic yellow fever, Syphilis, Systemic Inflammatory Response Syndrome (SIRS), Tabes dorsalis (tertiary syphilis), Taeniasis, Taiga encephalitis, Tanner's disease, Tapeworm infections, Temporal lobe encephalitis, Temporal lobe encephalitis, tetani (Lock Jaw), Tetanus Infection, Threadworm infections, Thrush, Tick, Tick typhus, Tinea barbae, Tinea capitis, Tinea corporis, Tinea cruris, Tinea manuum, Tinea nigra, Tinea pedis, Tinea unguium, Tinea versicolor, Torulopsosis, Torulosis, Toxic Shock Syndrome, Toxoplasmosis, transmissible spongioform (CJD), Traveler's diarrhea, Trench fever 5, Trichinellosis, Trichomoniasis, Trichomycosis axillaris, Trichuriasis, Tropical Spastic Paraparesis (TSP), Trypanosomiasis, Tuberculosis (TB), Tuberculousis, Tularemia, Typhoid Fever, Typhus fever, Ulcus molle, Undulant fever, Urban yellow fever, Urethritis, Vaginitis, Vaginosis, Vancomycin Intermediate (VISA), Vancomycin Resistant (VRSA), Varicella, Venezuelan Equine encephalitis, Verruga peruana, Vibrio cholerae (Cholera), Vibriosis (Vibrio), Vincent's disease or Trench mouth, Viral conjunctivitis, Viral Meningitis, Viral meningoencephalitis, Viral rash, Visceral Larval Migrans, Vomito negro, Vulvovaginitis, Warts, Waterhouse, Weil's disease, West Nile Fever, Western equine encephalitis, Whipple's disease, Whipworm infection, White Piedra, Whitlow, Whitmore's disease, Winter diarrhea, Wolhynia fever, Wool sorters' disease, Yaws, Yellow Fever, Yersinosis, Yersinosis (Yersinia), Zahorsky's disease, Zika virus disease, Zoster, Zygomycosis, John Cunningham Virus (JCV), Human immunodeficiency virus (HIV), Influenza virus, Hepatitis B, Hepatitis C, Hepatitis D, Respiratory syncytial virus (RSV), Herpes simplex virus 1 and 2, Human Cytomegalovirus, Epstein-Barr virus, Varicella zoster virus, Coronaviruses, SARS-CoV-2, Poxviruses, Enterovirus 71, Rubella virus, Human papilloma virus, Streptococcus pneumoniae, Streptococcus viridans., Staphylococcus aureus (S. aureus), Methicillin-resistant Staphylococcus aureus (MRSA), Vancomycin-intermediate Staphylococcus aureus (VISA), Vancomycin-resistant Staphylococcus aureus (VRSA), Staphylococcus epidermidis (S. epidermidis), Clostridium Tetani, Bordetella pertussis, Bordetella paratussis, Mycobacterium, Francisella Tularensis, Toxoplasma gondii, Candida (C. albicans, C. glabrata, C. parapsilosis, C. tropicalis, C. krusei and C. lusitaniae) and/or any other infectious diseases, disorders or syndromes.

Various infectious agents may be treated with pharmaceutical compositions, lipoprotein system, or polypeptides of the present disclosure. Non-limited examples of infectious agents include adenoviruses, Anaplasma phagocytophilium, Ascaris lumbricoides, Bacillus anthracis, Bacillus cereus, Bacteroides sp, Barmah Forest virus, Bartonella bacilliformis, Bartonella henselae, Bartonella quintana, beta-toxin of Clostridium perfringens, Bordetella pertussis, Bordetella parapertussis, Borrelia burgdorferi, Borrelia miyamotoi, Borrelia recurrentis, Borrelia sp., Botulinum toxin, Brucella sp., Burkholderia pseudomallei, California encephalitis virus, Campylobacter, Candida albicans, chikungunya virus, Chlamydia psittaci, Chlamydia trachomatis, Clonorchis sinensis, Clostridium difficile bacteria, Clostridium tetani, Colorado tick fever virus, Corynebacterium diphtheriae, Corynebacterium minutissimum, Coxiella burnetii, coxsackie A, coxsackie B, Crimean-Congo hemorrhagic fever virus, cytomegalovirus, dengue virus, Eastern Equine encephalitis virus, Ebola viruses, echovirus, Ehrlichia chaffeensis., Ehrlichia equi., Ehrlichia sp., Entamoeba histolytica, Enterobacter sp., Enterococcus faecalis, Enterovirus 71, Epstein-Barr virus (EBV), Erysipelothrix rhusiopathiae, Escherichia coli, Flavivirus, Fusobacterium necrophorum, Gardnerella vaginalis, Group B streptococcus, Haemophilus aegyptius, Haemophilus ducreyi, Haemophilus influenzae, hantavirus, Helicobacter pylori, Hepatitis A, Hepatitis B, Hepatitis C, Hepatitis D, Hepatitis E, herpes simplex virus 1 and 2, human herpes virus 6, human herpes Virus 8, human immunodeficiency virus 1 and 2, human T-cell leukemia viruses I and II, influenza viruses (A, B, C), Jamestown Canyon virus, Japanese encephalitis antigenic, Japanese encephalitis virus, John Cunningham virus, juninvirus, Kaposi's Sarcoma-associated Herpes Virus (KSHV), Klebsiella granulomatis, Klebsiella sp., Kyasanur Forest Disease virus, La Crosse virus, Lassavirus, Legionella pneumophila, Leptospira interrogans, Listeria monocytogenes, lymphocytic choriomeningitis virus, lyssavirus, Machupovirus, Marburg virus, measles virus, MERS coronavirus (MERS-CoV), Micrococcus sedentarius, Mobiluncus sp., Molluscipoxvirus, Moraxella catarrhalis, Morbilli-Rubeola virus, Mumpsvirus, Mycobacterium leprae, Mycobacterium tuberculosis, Mycobacterium ulcerans, Mycoplasma genitalium, Mycoplasma sp, Nairovirus, Neisseria gonorrhoeae, Neisseria meningitidis, Nocardia, Norwalk virus, norovirus, Omsk hemorrhagic fever virus, papilloma virus, parainfluenza viruses 1-3, parapoxvirus, parvovirus B19, Peptostreptococccus sp., Plasmodium sp., polioviruses types I, 11, and III, Proteus sp., Pseudomonas aeruginosa, Pseudomonas pseudomallei, Pseudomonas sp., rabies virus, respiratory syncytial virus, ricin toxin, Rickettsia australis, Rickettsia conori, Rickettsia honei, Rickettsia prowazekii, Ross River Virus, rotavirus, rubellavirus, Saint Louis encephalitis, Salmonella Typhi, Sarcoptes scabiei, SARS-associated coronavirus (SARS-CoV or SARS-CoV2), Serratia sp., Shiga toxin and Shiga-like toxin, Shigella sp., Sin Nombre Virus, Snowshoe hare virus, Staphylococcus aureus, Staphylococcus epidermidis, Streptobacillus moniliformis, Streptococcus pneumoniae, Streptococcus agalactiae, Streptococcus agalactiae, Streptococcus group A-H, Streptococcus pneumoniae, Streptococcus pyogenes, Treponema pallidum subsp. Pallidum, Treponema pallidum var. carateum, Treponema pallidum var. endemicum, Tropheryma whippelii, Ureaplasma urealyticum, Varicella-Zoster virus, variola virus, Vibrio cholerae, West Nile virus, yellow fever virus, Yersinia enterocolitica, Yersinia pestis, and Zika virus.

Various ocular diseases may be treated with pharmaceutical compositions, lipoprotein system, or polypeptides of the present disclosure. As a non-limiting example, the ocular disease may be thyroid eye disease (TED), Graves' disease (GD) and orbitopathy, Retina Degeneration, Cataract, optic atrophy, macular degeneration, Leber congenital amaurosis, retinal degeneration, cone-rod dystrophy, Usher syndrome, leopard syndrome, photophobia, and photoaversion.

### Inflammatory Disorders and Inflammation

In some embodiments, the compositions of the disclosure may be used to treat inflammation and/or inflammatory diseases. The inflammation may be neuroinflammation.

In some embodiments, the inflammation may be progressive. As a non-limiting example, the inflammation may be or may be caused by spinal cord compression or chronic traumatic encephalopathy. The lipoprotein systems and methods of using the lipoprotein systems described in the present disclosure may be used to prevent, manage and/or treat inflammatory disease. In some embodiments, the polypeptides and/or lipoprotein systems of the disclosure may be used to prevent, manage and/or treat inflammatory disease by clearing lipid debris, repairing cells, or restoring lipid homeostasis. Non-limiting examples of inflammatory diseases include Alzheimer's disease, multiple sclerosis (MS), ankylosing spondylitis, arthritis (osteoarthritis, rheumatoid arthritis (RA), psoriatic arthritis), asthma, atherosclerosis, Crohn's disease, colitis, dermatitis, diverticulitis, fibromyalgia, hepatitis, irritable bowel syndrome (IBS), systemic lupus erythematous (SLE), nephritis, Parkinson's disease, and ulcerative colitis. Many inflammatory disorders are severe, and even life-threatening. Antibodies targeting proteins associated with inflammation may be used to prevent, manage or treat inflammatory disorders as well as inflammation associated diseases.

### Injury

In some embodiments, the compositions of the disclosure may be used to treat or prevent injury. The injury may occur or may be associated with any organ or system of the body. For example, the injuries may be soft tissue injuries, orthopedic injuries, traumatic brain injuries and/or spinal cord injuries. In some embodiments, the brain injury may be a concussion, a contusion, a brain hemorrhage, an intracranial hematoma, coup-contrecoup brain injury, diffuse axonal injury or a penetrating brain injury. In some embodiments, the injury may be a spinal cord injury. In some embodiments, the spinal cord injury may be incomplete or complete. As a non-limiting example, the spinal cord injury may be anterior cord syndrome, central cord syndrome, Brown-Sequard syndrome, tetraplegia, paraplegia, or triplegia.

In some embodiments, the compositions of the disclosure may be used to treat Degenerative cervical myelopathy (DCM) (Desimone, A., et al. 2021. JCI insight, 6(15); the contents of which are herein incorporated by reference in its entirety). DCM is the most common cause of nontraumatic spinal cord injury in adults worldwide. Surgical decompression is generally effective in improving neurological outcomes and halting progression of myelopathic deterioration. However, a subset of patients experience suboptimal neurological outcomes. Functional analysis of DCM mouse model in targeted-replacement mice expressing human APOE4 revealed delayed gait recovery, forelimb grip strength, and hind limb mechanical sensitivity after decompression surgery, compared with their APOE3 counterparts. These data suggest the interplay of APOE in DCM phenotype. In some embodiments, compositions of the disclosure may be provided to subject with APOE3 or APOE4 genotype.

### Preventative Applications

The pharmaceutical compositions, lipoprotein system, including the polypeptides, lipids, and payloads of the present disclosure may be used to prevent the occurrence of a disease or prevent the progression of disease. In some embodiments, the lipoprotein system of the present disclosure may be provided to a subject prophylactically. In some embodiments, the lipoprotein system of the present disclosure is provided to a subject to treat a disease or disorder described herein. The subject may have the disease or disorder or may be at-risk to developing the disease or disorder. In some embodiments, the lipoprotein system of the present disclosure may be provided to a subject as part of an active immunization strategy to protect against diseases and disorders. In an active immunization strategy, the payloads of the lipoprotein system may be administered to a subject to prevent an infectious disease by activating the subject's immune response. In some embodiments, the lipoprotein system of the present disclosure is provided to the subject as part of a passive immunization strategy. In a passive immunization strategy. For example, lipoprotein systems with antibody payloads may be provided to a subject.

In some embodiments, the lipoprotein systems of the disclosure may be used for delivering payloads to subjects in need for the prevention of a particular disease. In some embodiments, the payloads may be vaccines, antigens and/or antibodies.

### Research Applications

The pharmaceutical compositions, lipoprotein system, and polypeptides therein may also be used as research tools. The lipoprotein system of the disclosure may be used as in any research experiment, e.g., in vivo or in vitro experiments. In a non-limiting example, the lipoprotein system of the disclosure may be used in cultured cells. The cultured cells may be derived from any origin known to one with skill in the art, and may be as non-limiting examples, derived from a stable cell line, an animal model or a human patient or control subject. In a non-limiting example, the lipoprotein system of the disclosure may be used in in vivo experiments in animal models (i.e., mouse, rat, rabbit, dog, cat, non-human primate, guinea pig, ferret, c-elegans, drosophila, zebrafish, or any other animal used for research purposes, known in the art). In another non-limiting example, the lipoprotein system of the disclosure may be used in human research experiments or human clinical trials.

### Combinatorial Applications

The lipoprotein systems of the disclosure may be used as a combination therapy with any other therapeutic modality known in the art. The additional therapeutic modality may be tailored to the disease being treated. In some embodiments, the cotherapy is a nucleic acid. Non-limiting examples of nucleic acids including gene silencing or interference (i.e., miRNA, siRNA, RNAi, shRNA), gene editing (i.e., TALEN, CRISPR/Cas9 systems, zinc finger nucleases), and gene, protein or enzyme replacement and/or antibody therapy (including monoclonal antibody therapy). In some embodiments, the cotherapy may be a small molecule.

For example, drugs for neurodegenerative diseases may be described with a lipoprotein system of the present disclosure (e.g., either as a payload or as a separate administration) for the treatment of a neurodegenerative disease. The following sections detail exemplary diseases and therapeutic modalities which may be combined with lipoprotein systems of the present disclosure.

### Neurological Disease Co-Therapy

In some embodiments, the neurodegenerative disease may be one or more of the diseases described herein (e.g., those listed in the section “Neurological Diseases” above). In some embodiments, the lipoprotein systems of the present disclosure may be combined with, e.g., an anti-amyloid agent, for example, the lipoprotein systems of the present disclosure may be combined with an anti-amyloid antibody (i.e., an anti-amyloid agent, wherein the agent is an antibody) (e.g., aducanumab, lecanemab, solanezumab, donanemab, gantenerumab), a small molecule (e.g., levetriactem, sodium oligomannate, donprezil, galantamine, rivastigmine, or memantine), or a further combination therapy (e.g., chromolyn sodium/ibuprofen) (see, e.g., Lozupone et al. Anti-amyloid-3 protein agents for the treatment of Alzheimers disease: an update on emerging drugs, 2020 (3): 319-335); “Treatment and Research.” Alzheimer's Disease and Dementia, www.alz.orghelp-support/i-have-alz/treatments-research; and Bateman et al. Gantenerumab: an anti-amyloid monoclonal antibody with potential disease-modifying effects in early Alzheimer's disease; the entirety of which are incorporated herein by reference). In some embodiments, the neurological disease is Amyloid Related Imaging Abnormalities (ARIA) or Alzheimer's disease.

### Oncology Co-Therapy

Neuropathy is a frequent side effect for cancer survivors. The lipoprotein systems of the disclosure may be used for neuroprotection as a combination therapy with oncology treatment, which may include surgery, radiation, chemotherapy, biological therapy, and any other therapeutic modality known in the art. The lipoprotein systems can be used to prevent, treat, or ameliorate neurodegeneration associated with tumor growth or induced by oncology treatment. As a non-limiting example, the lipoprotein systems may be used in combination with cytostatic drugs (e.g., docetaxel, capecitabine, gemcitabine, irinitocan, ixabepilone or pemetrexed), small molecule inhibitors, anti-tumor antibodies (cetuximab, trastuzumab, panitumumab (targets include HER-1, HER-2 and RAS)), anti-angiogenesis antibodies, CAR-T cells, vaccines, oncolytic viruses (e.g., RNA viruses, including Newcastle Disease Virus from attenuated natural wild type strains).

### Kits

The disclosure provides kits including the compositions as described herein, or the pharmaceutical composition as described herein. The kits can further include one or more additional aria therapeutic regimens or agents.

Also disclosed herein, in some embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In some embodiments, the containers are formed from a variety of materials such as glass or plastic.

The articles of manufacture provided herein contain packaging materials. Pharmaceutical packaging materials include, but are not limited to, blister packs, bottles, tubes, bags, containers, bottles, and any packaging material suitable for a selected formulation and intended mode of administration and treatment.

For example, the container(s) include the composition of the disclosure, and optionally in addition with therapeutic regimens or agents disclosed herein. Such kits optionally include an identifying description or label or instructions relating to its use in the methods described herein.

A kit typically includes labels listing contents and/or instructions for use, and package inserts with instructions for use. A set of instructions will also typically be included.

In some embodiments, a label is on or associated with the container. In some embodiments, a label is on a container when letters, numbers or other characters forming the label are attached, molded or etched into the container itself; a label is associated with a container when it is present within a receptacle or carrier that also holds the container, e.g., as a package insert. In some embodiments, a label is used to indicate that the contents are to be used for a specific therapeutic application. The label also indicates directions for use of the contents, such as in the methods described herein.

The present disclosure is further illustrated by the following non-limiting examples.

### EXAMPLES

### Example 1: Design and Evaluation of Exemplary Lipoprotein Systems

### Objective

The present example is directed toward the synthesis and characterization of exemplary lipoprotein systems of the present invention. The present example is directed towards combinations of truncation and point mutation modifications of APOE and discovered novel variants with enhanced biophysical stability and preserved lipid efflux capacity.

### Methods

DNA sequences encoding the amino acid constructs identified by PP85 (SEQ ID NO: 348), PP86 (SEQ ID NO: 349), PP87 (SEQ ID NO: 350), PP64 (SEQ ID NO: 2), PP65 (SEQ ID NO:1) and PP66 (SEQ ID NO: 3) were cloned into the pcDNA3.4 vector and transiently transfected into Expi293 suspension cells (ThermoFisher). Secreted polypeptides were harvested between 3 days and 14 days post-transfection under denaturing conditions to minimize aggregation. During secretion, the signal strand (SS) of the polypeptide sequence is cleaved, resulting in final sequences identified as PP9 (SEQ ID NO: 275) PP10 (SEQ ID NO: 276), PP11 (SEQ ID NO: 277), PP1 (SEQ ID NO: 268), PP2 (SEQ ID NO: 267) and PP3 (SEQ ID NO: 269). Suspension cells were pelleted by centrifugation (1,000×g, 5 minutes), and the pellet was discarded. The proteins were precipitated from the supernatant by addition of 1 M MnCl2 in water to a final concentration of 0.1 M MnCl2 followed by incubation at 4° C. for 1-18 hours. The precipitated proteins were pelleted by centrifugation (3,000×g, 10 minutes) and the pellet was resuspended in resolubilization buffer (6 M guanidinium HCl (GdnHCl), 15 mM sodium phosphate, 50 mM TCEP, pH 8) with vortexing and nutation for 1 hour at room temperature. Residual salts and aggregated proteins were removed by centrifugation (3,000×g, 10 minutes), and the solubilized proteins in the supernatant were purified by buffer exchange and ion exchange chromatography (IEX). For example, the proteins may be buffer exchanged into low salt buffer (20 mM Tris HCl, 20 mM NaCl, pH 7.5) on HiTrap Desalting columns (GE), loaded onto a strong anion exchange resin such as a HiTrap Q HP anion exchange column (GE), and eluted with a gradient of high salt buffer (20 mM Tris HCl, 1 M NaCl, pH 7.5). Following IEX, a 10 kDa molecular weight cutoff centrifugal filter (Amicon or Sartorius) was used to concentrate the polypeptides and the buffer was exchanged to a denaturing buffer (6 M GdnHCl, 15 mM sodium phosphate, 20 mM MOPS). The polypeptides were stored at a concentration of 3-5 mg/mL in buffer conditions that minimize multimerization/aggregation and oxidation. The purity of the polypeptides was assessed by reducing and non-reducing sodium dodecyl sulfate (SDS) polyacrylamide gel electrophoresis (PAGE) with 4-12% Bis-Tris NuPAGE gels (ThermoFisher) and staining with Aquastain (Bulldog). Sequences were confirmed by mass spectrometry.

Lipoprotein systems were synthesized using 1-palmitoyl-2-oleoyl-sn-glycero-3-phosphocholine (POPC) and the polypeptides described above. The organic solvent surrounding the lipid was evaporated under a stream of N2, and residual solvent was further removed by triturating with acetone, evaporating the volatiles under a stream of N2, and removing residual solvent under reduced pressure. The dried lipids were resuspended in lipid resuspension buffer (20 mM MOPS, 100 mM NaCl, 2% cholate w/v, pH 7.0). Lipids were quantified prior to assembly of exemplary lipoprotein systems using enzyme-coupled reactions with a standard curve.

Each of the polypeptides were diluted in denaturing buffer and mixed with the lipid POPC solubilized in resuspension buffer such that the final concentration of polypeptides was 0.4-1.3 mg/mL. The fully reconstituted reaction mixtures were then allowed to incubate on ice for 30-60 minutes. After incubation, the reactions were transferred into the appropriate volume dialysis chamber; for example, a 96-well dialysis plate (ThermoFisher) for small-scale screening reactions, or individual dialysis cassettes (ThermoFisher) for larger scale reactions. Detergent and denaturant were removed by dialysis for at least 20 hours against assembly buffer (20 mM MOPS, 100 mM NaCl, pH 7.0), with 1 exchange of assembly buffer at a volume at least 500-fold greater than the total assembly reaction volume. A polystyrene adsorbent resin (e.g., Bio-Rad BioBeads SM-2) was added to the dialysate upon exchange of the assembly buffer. The dialysis process was performed at 4° C.

### Results

The assembled lipoprotein systems LPS1 including SEQ ID No: 275 (PP9) and POPC, LPS2 including SEQ ID No: 276 (PP10) and POPC, LPS3 including SEQ ID No: 277 (PP11), LPS4 including APOE2 (SEQ ID NO: 267 (PP1)) and POPC; LPS5 including APOE 3 (SEQ ID NO: 268 (PP2) and POPC, and LPS6 including APOE4 (SEQ ID NO: 269 (PP3)) and POPC) were assessed by Native PAGE immediately after assembly (0.8 mg-1.25 mg scale) and stained for total protein using AcquaStain (Bulldog Bio). The results demonstrated successful disc formation of LPS1, LPS2 and LPS3 as indicated by the characteristic patterns of narrow, intense bands that vary according to polypeptide: lipid ratio, similar to that seen with lipoprotein systems comprising the native apolipoprotein isoforms (i.e., LPS4, LPS5 and LPS6). These data informed the selection of optimal polypeptide: lipid ratios for larger scale disc assembly reactions. Further, LPS1, LPS2 and LPS3 yielded similarly sized discs to those evident for lipoprotein systems comprising the natural human APOE isoforms APOE2, APOE3 and APOE4.

As additional confirmation of particle size, lipoprotein systems LPS1, LPS2, LPS3, LPS4, LPS5 and LPS6 were diluted at 100 μg/mL in phosphate buffered saline (PBS) and measured using dynamic light scattering (DLS) across ten acquisitions of ten seconds each using a Wyatt DynaPro Plate Reader III at 25° C. DLS offered several advantages over the Native PAGE methods by providing rapid and quantitative assessment of lipoprotein system size. Data are shown below in Table 9.

TABLE 9
Dynamic Light Scattering and Transmission Electron Microscopy

| | | Dynamic Light | | | |
|---|---|---|---|---|---|
| | | Scattering (DLS) | | Transmission Electron | |
| | Poly- | | Poly- | Microscopy (TEM) | |
| | peptide:Lipid | Diameter | dispersity | Diameter | Standard |
| Particle | (mol/mol) | (nm) | (nm) | (nm) | dev (nm) |
| LPS1 | 1:105 | 15.9 | 0.32 | 19.8 | 5.4 |
| LPS2 | 1:100 | 12.9 | 1.4 | 18.8 | 3.1 |
| LPS3 | 1:90 | 14.9 | 0.87 | 19.5 | 5.4 |
| LPS4 | 1:110 | 18.3 | 0.47 | 21.2 | 4.5 |
| LPS5 | 1:95 | 14.3 | 1.7 | 19.6 | 3.5 |
| LPS6 | 1:125 | 10.8 | 1.27 | 16.1 | 3.1 |

The data of the DLS assay demonstrated that LPS1, LPS2, and LPS3 had similar particle diameters to LPS4, LPS5, LPS6.

The lipoprotein systems (10 μg/mL) were then stained with uranyl acetate and imaged by transmission electron microscopy (TEM) for further evaluation and characterization. The assessment by TEM showed that all six preparations (LPS1, LPS2, LPS3, LPS4, LPS5 and LPS6) presented the expected discoidal morphology. Particle diameters were measured and the data are shown in Table 9 above. A comparison of the particle diameter measurements as assessed by DLS and TEM yielded a strong correlation with R²=0.91.

### Conclusions:

Synthetic APOEs are capable of forming HDL particles.

### Example 2: Comparison of Biophysical Properties of Lipoprotein Systems

### Objective

The present example is directed towards assessing the different biophysical properties of exemplary lipoprotein systems of the present invention.

### Methods

ExpiCHO and/or Expi293 suspension cells are subcultured according to the manufacturer's protocol (ThermoFisher). Inserts encoding the compounds of polypeptides are cloned into the pcDNA3.4 vector and feature the signal sequences described herein by GeneArt/ThermoFisher. Cells are transiently transfected by complexing purified plasmids with a complexing reagent such as ExpiFectamine (ThermoFisher). Tissue culture compatible protease inhibitors (Sigma-Aldrich) may be added every other day starting at 24 hours post-transfection, and the secreted polypeptides may be harvested between 5 days and 14 days post-transfection. In some instances, the polypeptides may be expressed as an intein fusion protein.

Suspension cells are pelleted by centrifugation (1,000×g, 5 minutes), and the pellet is discarded. The proteins may be precipitated from the supernatant by addition of 1 M MnCl2 in water to a final concentration of 0.1 M MnCl₂ followed by incubation at 4° C. for 1-18 hours in the presence of protease inhibitors (Roche). The precipitated proteins are pelleted by centrifugation (3,000×g, 10 minutes) and the pellet is resuspended in resolubilization buffer (6 M guanidinium HCl (GdnHCl), 15 mM sodium phosphate, 50 mM TCEP, pH 8) with vortexing and nutation for 1 hour at room temperature. Residual salts and aggregated proteins are removed by centrifugation (3,000×g, 10 minutes), and the solubilized proteins in the supernatant are purified by buffer exchange and ion exchange chromatography (IEX). For example, the proteins may be buffer exchanged into low salt buffer (20 mM Tris HCl, 20 mM NaCl, pH 7.5) on HiTrap Desalting columns (GE), loaded onto a strong anion exchange resin such as a HiTrap Q HP anion exchange column (GE), and eluted with a linear gradient of high salt buffer (20 mM Tris HCl, 1 M NaCl, pH 7.5). Following IEX, a 10 kDa molecular weight cutoff centrifugal filter (Amicon or Sartorius) is used to concentrate the polypeptides and exchange the buffer to denaturing buffer (6 M GdnHCl, 15 mM sodium phosphate, 20 mM MOPS). Lastly, the purified polypeptides may be optionally reduced with 50 mM TCEP and polished by size-exclusion chromatography (SEC, for example a Superdex 75 column, GE) with isocratic elution in denaturing buffer. Alternately, the polypeptides in the clarified media may be directly purified by IEX or another affinity-based chromatography method with or without a final SEC step. The polypeptides are stored at a concentration of 3-5 mg/mL in buffer conditions that minimize multimerization/aggregation and oxidation (for example, denaturing buffer+50 mM TCEP). The polypeptides that have been intentionally oxidized to form disulfide bonds are not stored in the presence of TCEP. The purity of the compounds of polypeptides is assessed by reducing and non-reducing sodium dodecyl sulfate (SDS) polyacrylamide gel electrophoresis (PAGE) with 4-12% Bis-Tris NuPAGE gels (ThermoFisher) and staining with SimplyBlue (ThermoFisher).

For the assembly of lipoprotein systems, aliquots of purified polypeptide were allowed to thaw at 2-8° C. Aqueous 0.5 M tris(2-carboxyethyl)phosphine (TCEP) solution was then added to the polypeptide solution (1.5 μL per 200 μg polypeptide) to reduce any disulfide bonds that formed during storage. After incubation for >30 minutes at 2-8° C., the mixture was buffer exchanged into protein storage buffer (6 M guanidinium HCl, 20 mM MOPS, pH 7.0) using a spin desalting column (ThermoFisher) following the manufacturer's instructions. Optionally, TCEP was removed via dialysis. After buffer exchange, the concentration of the polypeptide was determined by measuring the sample's absorbance at 280 nm (A280) or performing a bicinchoninic acid (BCA) assay (ThermoFisher). If necessary, the polypeptide was diluted in protein storage buffer to the appropriate working concentration for subsequent experiments.

For lipids supplied as neat powders or oils (Acros Organics, Avanti, Corden Pharma, Sigma Aldrich, Thermo Scientific, VWR), the lipid was transferred to a tared glass vial or plastic tube and solubilized in lipid solubilization buffer (20 mM MOPS, 100 mM NaCl, 2% sodium cholate [w/v], pH 7.0). For lipids supplied as solutions in organic solvent (Avanti), the solution was transferred to a tared glass vial, the solvent was evaporated under a stream of nitrogen gas, and residual solvent was removed under reduced pressure. After >1 hour, the lipid film was triturated with acetone, and the volatiles were removed under a stream of nitrogen gas followed by reduced pressure. After >16 hours, the lipid film was solubilized in lipid solubilization buffer. Before they were used in subsequent experiments, the concentrations of the lipid solutions were quantified using an enzyme-coupled fluorometric assay (BioAssay Systems) or by measuring the mass of the tared vessel before solubilization.

For each polypeptide-lipid combination, the optimal protein:lipid ratio in the initial reaction mixture must be determined empirically. Unoptimized ratios could result in the presence of lipid-free polypeptide, free liposomes, or aggregates in the final sample and/or high polydispersity values for the disc species. Similarly, the temperatures at which various steps take place should be determined empirically to identify the conditions under which disc formation is most robust.

To generate the assembly reaction mixtures, the following components were combined sequentially in appropriately sized PCR or microcentrifuge tubes: 1) lipid solution(s) in lipid solubilization buffer (20 mM MOPS, 100 mM NaCl, 2% cholate [w/v], pH 7.0), 2) additional lipid solubilization buffer to ensure that the cholate concentration in the final reaction mixture would be 20-30 mM, 3) sufficient protein storage buffer (6 M guanidinium HCl, 20 mM MOPS, pH 7.0) to ensure that the guanidinium concentration in the final reaction mixture would be 3-3.5 M, and 4) the polypeptide solution in protein storage buffer such that the protein concentration in the final reaction mixture would be 0.25-3.0 mg/mL. After addition of each component, the solutions were mixed thoroughly by pipetting up and down several times. Variables that were analyzed systematically include total protein:lipid ratio and proportions of lipid components (if the mixture contained multiple lipids), among others.

The fully reconstituted reaction mixtures were allowed to incubate at an empirically determined temperature for 30-120 minutes; for assembly reactions containing a single phospholipid, the incubation temperature was often at least 2° C. greater than the phase transition temperature of the lipid. The solutions were then transferred to dialysis devices (ThermoFisher) that had been prepared according to the manufacturer's instructions. The guanidinium and cholate were then removed from the reaction mixtures via dialysis against >250-fold excess assembly buffer (20 mM MOPS, 100 mM NaCl, pH 7.0). Optionally, after >1 hour of dialysis, the dialysate was poured out and replaced with fresh assembly buffer and a volume of polystyrene adsorbent resin (Bio-Rad) equal to 0.25-2% of the volume of the dialysate. Optionally, the resin can be added to the dialysate at the initiation of dialysis and the buffer exchange can be skipped. After >20 hours of dialysis, the samples were retrieved from the dialysis devices. The temperature at which the dialysis process occurred was determined empirically based on the conditions that yielded the highest quality reaction products, which was usually in the range of 4-40° C.

Once the optimal assembly conditions for a given polypeptide-lipid combination were identified, large-scale assembly of the lipoprotein system (>500 μg input polypeptide) could take place. The methods for preparing the polypeptide, lipid, and assembly reaction mixture were analogous to those described above for screening-scale experiments. The guanidinium and cholate were then removed from the reaction mixture via dialysis against >250-fold excess assembly buffer (20 mM MOPS, 100 mM NaCl, pH 7.0). After >2 hours of dialysis, the dialysate was poured out and replaced with fresh assembly buffer and a volume of polystyrene adsorbent resin (Bio-Rad) equal to 0.25-2% of the volume of the dialysate. After >22 hours of total dialysis, the sample was retrieved from the dialysis device. The temperature at which the dialysis process occurred was determined empirically based on the conditions that yielded the highest quality reaction products. Optionally, to remove residual cholate after dialysis had finished, adsorbent resin (excess water removed) was added to the reaction mixture directly (˜50-200 mg of resin per 1 mg of protein) and agitated via magnetic stirring at 4° C. for >2 hours.

The reaction mixture was then concentrated using centrifugal spin concentrators (Sartorius) to <20% of its original volume in a refrigerated centrifuge held at 4° C. The sample was then diluted back to its original volume in PBS (2 mM KH₂PO₄, 10 mM Na₂HPO₄, 140 mM NaCl, 3 mM KCl, pH 7.4) and mixed via pipetting up and down several times. This process was repeated two additional times to achieve a sufficient degree of exchange into PBS. Optionally, the buffer exchange could be achieved by performing dialysis against a volume of PBS >250-fold greater than the volume of the disc solution.

The sample was then concentrated to the target concentration and filtered through a sterile syringe filter (pore size ≤0.22 μm) in a biosafety cabinet.

Several methods were used to assess lipoprotein quality, batch-to-batch variability, and the presence of lipid-free polypeptide or aggregates. First, the lipoprotein samples were analyzed via native PAGE analysis using 4-12% Tris-Glycine gels (Invitrogen) following the manufacturer's instructions. Gels were stained with SimplyBlue (ThermoFisher) or AcquaStain (BulldogBio) to visualize protein or Sudan Black (Sigma-Aldrich) to visualize lipids.

The diameters of the lipoproteins and polydispersity of the samples were measured using dynamic light scattering (DLS) following standard protocols. In brief, samples were diluted to 0.1-1 mg/mL (protein concentration), and >25 μL of each sample was added to a DLS-compatible 384-well plate (Aurora). The samples were then analyzed on a plate-based DLS instrument (Wyatt).

Both the diameter and morphology were also characterized by transmission electron microscopy (TEM). In brief, samples were diluted in PBS (2 mM KH₂PO₄, 10 mM Na₂HPO₄, 140 mM NaCl, 3 mM KCl, pH 7.4) to 0.025-0.2 mg/mL (protein concentration) and stained with 2% uranyl acetate. The prepared grids were analyzed on a TEM system (Hitachi) operating at an accelerating voltage of 100 kV. Images were captured at magnifications ranging from 20,000× to 80,000×. Lipoprotein size and size distribution were extracted from the images using the Analyze Particles function in ImageJ on the images with the highest degree of magnification.

The protein, phosphatidylcholine, and cholate contents of the lipoprotein samples were measured using enzyme-coupled fluorometric or colorimetric assays (Bio-Rad for the protein assay; BioAssay Systems for the phosphatidylcholine and cholate assays) calibrated with standard curves. The protein content of the lipoproteins was calculated by measuring the absorbance of the sample at 280 nm (A280) and using the extinction coefficient of the corresponding polypeptide.

A surface plasmon resonance (SPR) method was developed to assess the binding response between the lipoprotein samples and the low-density lipoprotein receptor (LDLR). This interaction is relevant to the discs because the LDLR/APOE pathway plays a pivotal role in cholesterol homeostasis and lipid metabolism. Experiments were performed on a Biacore 4000 with human LDLR protein captured on a Protein A chip. For the binding studies, lipoprotein samples were diluted and tested in a 3-fold dilution series. Samples were injected for 2-3 minutes at 30 μL/min and the dissociation phase was monitored for 5-10 minutes. Response data were processed by subtracting the data from a reference surface (i.e., without receptor captured) and a buffer injection. The processed data were globally fit to a 1:1 interaction model to extract estimates of the binding constants.

The thermal stability of lipoproteins was assessed via dynamic light scattering (DLS). In brief, samples were diluted in PBS (2 mM KH₂PO₄, 10 mM Na₂HPO₄, 140 mM NaCl, 3 mM KCl, pH 7.4) to 100-250 μg/mL, filtered (0.02 μm pore size), and loaded in technical duplicate, triplicate, or quadruplicate into a DLS-compatible 384-well plate (Aurora). The wells were inspected visually; if at least one of the wells contained a bubble, the plate was spun at 400-600 RCF for 1 minute in a refrigerated centrifuge held at 4° C. Translucent sealing tape was then placed over the wells to mitigate volume loss due to evaporation.

Once the DLS plate was fully prepared, it was added to a plate-based DLS instrument (Wyatt) that had been heated to 25° C. DLS measurements were then made continuously as the temperature rose from 25° C. to 75-85° C. at a ramp rate of 0.15-0.3° C. per minute. Optionally, the temperature was raised in discrete increments and DLS measurements were only performed once the temperature of the analysis chamber had stabilized at that temperature.

To track particle size as a function of temperature, the resulting data were processed using the DLS software's (Wyatt Dynamics 8.0) Temperature Dependence Analysis algorithm. The Onset Temperature for each sample was determined to gauge thermal stability, which is the temperature at which the particle radius began to increase exponentially. The algorithm parameters were set as follows:
Below Threshold (° C.): 10
Above Threshold (° C.): 10
Zero Slope: Yes
Threshold Percentage: 10

Optionally, for the discrete temperature ramp protocol, the temperature at which the samples began to decompose was identified by visual inspection of the size distribution plots.

To determine the susceptibility of lipoproteins to extended cold storage and repeated freeze-thaw cycles, lipoprotein samples were adjusted to 400 μg/mL via dilution in PBS (2 mM KH₂PO₄, 10 mM Na₂HPO₄, 140 mM NaCl, 3 mM KCl, pH 7.4). Each sample was then dispensed into separate microcentrifuge tubes, and the tubes were placed in a freezer held at −20° C. After 7 days, the tubes were removed from the freezer and allowed to thaw at ambient temperature over the course of 20 minutes, after which they were returned to the −20° C. freezer. This process was repeated 4 days later (11 days after initiation of the experiment) and 13 days later (20 days after initiation of the experiment).

After the contents of the tubes had thawed on day 11 (2 freeze-thaw cycles) and day 20 (3 freeze-thaw cycles), aliquots were removed for analysis via native PAGE and dynamic light scattering (DLS). In brief, native PAGE analysis was performed using 4-12% Tris-Glycine gels (Invitrogen) following the manufacturer's instructions. Gels were stained with AcquaStain (BulldogBio) to visualize protein. To perform DLS, samples were diluted 2-fold in PBS and 25 μL of each sample was added to a DLS-compatible 384-well plate (Aurora). The samples were then analyzed on a plate-based DLS instrument (Wyatt). The same analyses had also been performed on the diluted samples before they were initially introduced into the −20° C. freezer to serve as a baseline. The resulting time series data were analyzed.

### Results

Prior examples were directed to the structure of discs formed with POPC. It was desired to compare the properties of PC lipids with alternative classes of lipids. To that end, LPS 7-33 were synthesized through the methods described herein using PP2 and POPC, DMPC, POPS, or POPG. The results are summarized in Table 10.

TABLE 10
Physical Properties of Discs formed with PP2 and various lipids

| | | | Disc | | |
|---|---|---|---|---|---|
| | | PP2:Lipid | Diameter | Disc % | Disc |
| Particle | Lipid | Ratio | (nm) | Polydispersity | Intensity % |
| LPS7 | POPC | 1:65 | 14 | 11.8 | 63.3 |
| LPS8 | POPC | 1:80 | 16.3 | 33.5 | 82.5 |
| LPS5 | POPC | 1:95 | 15.6 | 25 | 63.1 |
| LPS9 | POPC | 1:110 | 14.4 | 10.1 | 58.8 |
| LPS10 | POPC | 1:125 | 14.4 | 12.7 | 65.3 |
| LPS11 | POPC | 1:140 | 16.3 | 19.8 | 53.2 |
| LPS12 | DMPC | 1:100 | 13.8 | 21.2 | 62.2 |
| LPS13 | DMPC | 1:120 | 15.9 | 29.2 | 97.7 |
| LPS14 | DMPC | 1:140 | 12.7 | 13.1 | 58.0 |
| LPS15 | DMPC | 1:160 | 13.9 | 17.6 | 83.8 |
| LPS16 | DMPC | 1:180 | 13.6 | 20.7 | 71.7 |
| LPS17 | DMPC | 1:200 | 12.2 | 4.9 | 88.2 |
| LPS18 | DMPC | 1:220 | 13.4 | 11.1 | 82.5 |
| LPS19 | DMPC | 1:240 | 13.9 | 7.4 | 88.0 |
| LPS20 | POPG | 1:40 | 10.2 | 7.1 | 85.4 |
| LPS21 | POPG | 1:60 | 10.9 | 5.2 | 91.1 |
| LPS22 | POPG | 1:80 | 11.4 | 7.4 | 88.1 |
| LPS23 | POPG | 1:100 | 12 | 9.8 | 91.2 |
| LPS24 | POPG | 1:120 | 13.8 | 8 | 94.9 |
| LPS25 | POPG | 1:140 | 13.8 | 2 | 95.9 |
| LPS26 | POPG | 1:160 | 14.8 | 4.7 | 92.7 |
| LPS27 | POPS | 1:50 | 8.1 | 21.4 | 49.1 |
| LPS28 | POPS | 1:70 | 10.0 | 17.0 | 85.9 |
| LPS29 | POPS | 1:90 | 8.6 | 6.2 | 72.4 |
| LPS30 | POPS | 1:110 | 9.1 | 11.8 | 87.0 |
| LPS31 | POPS | 1:130 | 10.0 | 35.5 | 78.5 |
| LPS32 | POPS | 1:150 | 10.6 | 24.5 | 77.2 |

Particles containing POPG (LPS 20-26) were found to consistently have a more uniform size distribution (i.e., lower polydispersity %) than lipoprotein systems containing other lipids with a similar polypeptide:lipid ratio. Additionally, particles containing POPS (LPS 27-32) were found to consistently have a smaller hydrodynamic diameter than lipoprotein systems containing other lipids.

Building on these results, particles were made with PP4 (SEQ ID NO: 270) and POPC or POPG. The results are summarized in Table 11

TABLE 11
Physical Properties of Discs formed with PP4

| | | | Disc | | |
|---|---|---|---|---|---|
| | | PP4:lipid | Diameter | Disc % | Disc |
| Particle | Lipid | ratio | (nm) | Polydispersity | Intensity % |
| LPS33 | POPC | 1:40 | 13.6 | 12.5 | 90 |
| LPS34 | POPC | 1:60 | 13.9 | 7.4 | 89.1 |
| LPS35 | POPC | 1:80 | 14.3 | 10.5 | 91 |
| LPS36 | POPC | 1:100 | 15.1 | 3.6 | 95 |
| LPS37 | POPC | 1:120 | 17.7 | 6.6 | 97.3 |
| LPS38 | POPC | 1:140 | 17.7 | 7.7 | 96.4 |
| LPS39 | POPC | 1:160 | 20 | 11.3 | 97 |
| LPS40 | POPG | 1:40 | 10.2 | 6.7 | 100 |
| LPS41 | POPG | 1:60 | 10.6 | 4.7 | 97 |
| LPS42 | POPG | 1:80 | 13.4 | 4.7 | 98.7 |
| LPS43 | POPG | 1:100 | 14.4 | 5.4 | 97.8 |
| LPS44 | POPG | 1:120 | 14.4 | 5 | 96.8 |
| LPS45 | POPG | 1:140 | 16.6 | 5.6 | 100 |
| LPS46 | POPG | 1:160 | 19.2 | 20.2 | 100 |

Particles containing PP5 and POPC or POPG were then examined. The results are summarized in Table 12.

TABLE 12
Physical Properties of Discs formed with PP5

| | | | Disc | | |
|---|---|---|---|---|---|
| | | PP5:Lipid | Diameter | Disc % | Disc |
| Particle | Lipid | Ratio | (nm) | Polydispersity | Intensity % |
| LPS47 | POPC | 1:40 | 12.4 | 3.1 | 91.9 |
| LPS48 | POPC | 1:60 | 11.7 | 13 | 86.9 |
| LPS49 | POPC | 1:80 | 11.4 | 6.6 | 93.9 |
| LPS50 | POPC | 1:100 | 12.7 | 3.2 | 93.4 |
| LPS51 | POPC | 1:120 | 14.6 | 5 | 98.9 |
| LPS52 | POPC | 1:140 | 14.9 | 4.4 | 96.6 |
| LPS53 | POPC | 1:160 | 16.5 | 9.5 | 98.2 |
| LPS54 | POPG | 1:40 | 10.9 | 7.6 | 79.5 |
| LPS55 | POPG | 1:60 | 11.5 | 13.5 | 73.8 |
| LPS56 | POPG | 1:80 | 12.2 | 11.2 | 84.5 |
| LPS57 | POPG | 1:100 | 11.5 | 5 | 94.5 |
| LPS58 | POPG | 1:120 | 13 | 6 | 93.1 |
| LPS59 | POPG | 1:140 | 14.3 | 12.1 | 90.4 |
| LPS60 | POPG | 1:160 | 15.1 | 12.8 | 96.6 |
| LPS61 | POPG | 1:180 | 16.9 | 23.9 | 97.7 |

Particles containing variant apolipoproteins PP10 or PP11, and POPC, POPG, DMPC, or N-oleoyl-D-erythro-sphingosylphosphorylcholine (18:1 SM) were then examined. The results are summarized in Table 13.

TABLE 13
Physical Properties of Discs formed with Variant Apolipoproteins

| | | | | Disc | Disc | Disc |
|---|---|---|---|---|---|---|
| | | | Apolipopro- | Diam- | % | Inten- |
| Par- | Apolipo- | | tein:Lipid | eter | Polydis- | sity |
| ticle | protein | Lipid | Ratio | (nm) | persity | % |
| LPS62 | PP10 | POPC | 1:38 | 5.4 | 13.9 | 74.2 |
| LPS63 | PP10 | POPC | 1:51 | 6.2 | 18.2 | 78.7 |
| LPS64 | PP10 | POPC | 1:64 | 6.0 | 8.2 | 89.5 |
| LPS65 | PP10 | POPC | 1:76 | 5.8 | 18.4 | 85.5 |
| LPS66 | PP10 | POPC | 1:89 | 6.6 | 20.4 | 83.3 |
| LPS67 | PP10 | POPC | 1:101 | 7.3 | 5.0 | 90.5 |
| LPS68 | PP10 | POPC | 1:114 | 7.8 | 7.8 | 95.5 |
| LPS69 | PP10 | POPC | 1:127 | 7.6 | 2.7 | 96.3 |
| LPS70 | PP11 | POPC | 1:38 | 10.2 | 14.4 | 67.1 |
| LPS71 | PP11 | POPC | 1:51 | 10.2 | 16.8 | 74.7 |
| LPS72 | PP11 | POPC | 1:64 | 12.8 | 5.4 | 82.2 |
| LPS73 | PP11 | POPC | 1:76 | 14.0 | 20.2 | 79.2 |
| LPS74 | PP11 | POPC | 1:89 | 15.2 | 11.5 | 91.4 |
| LPS75 | PP11 | POPC | 1:101 | 16.6 | 14.4 | 91.8 |
| LPS76 | PP11 | POPC | 1:114 | 15.8 | 14.5 | 91.7 |
| LPS77 | PP11 | POPC | 1:127 | 16.4 | 20.1 | 87.6 |
| LPS78 | PP10 | DMPC | 1:70 | 13.8 | 24.5 | 85.1 |
| LPS79 | PP10 | DMPC | 1:110 | 11.4 | 21.7 | 78.7 |
| LPS80 | PP10 | DMPC | 1:150 | 12.6 | 17.2 | 84.5 |
| LPS81 | PP10 | DMPC | 1:190 | 11.2 | 27.2 | 70.3 |
| LPS82 | PP11 | DMPC | 1:70 | 12.6 | 35.7 | 80.4 |
| LPS83 | PP11 | DMPC | 1:110 | 8.8 | 10.0 | 75.7 |
| LPS84 | PP11 | DMPC | 1:150 | 10.2 | 19.5 | 51.6 |
| LPS85 | PP11 | DMPC | 1:190 | 10.0 | 9.3 | 81.8 |
| LPS86 | PP10 | 18:1 SM | 1:80 | 10.8 | 13.7 | 76.8 |
| LPS87 | PP10 | 18:1 SM | 1:120 | 12.1 | 16.2 | 75.6 |
| LPS88 | PP10 | 18:1 SM | 1:160 | 12 | 14 | 68.4 |
| LPS89 | PP10 | 18:1 SM | 1:200 | 13.7 | 14.1 | 70.5 |
| LPS90 | PP11 | 18:1 SM | 1:80 | 11.3 | 14.9 | 55.2 |
| LPS91 | PP11 | 18:1 SM | 1:120 | 12.7 | 15.4 | 45.8 |
| LPS92 | PP11 | 18:1 SM | 1:160 | 13.1 | 13.7 | 64.8 |
| LPS93 | PP11 | 18:1 SM | 1:200 | 14.7 | 16.6 | 71.4 |
| LPS94 | PP10 | POPG | 1:40 | 9.2 | 7.8 | 89.5 |
| LPS95 | PP10 | POPG | 1:60 | 10.9 | 5.5 | 90.7 |
| LPS96 | PP10 | POPG | 1:80 | 11.8 | 4.9 | 97 |
| LPS97 | PP10 | POPG | 1:100 | 12.1 | 4.8 | 92.4 |
| LPS98 | PP10 | POPG | 1:120 | 14 | 7.1 | 95.7 |
| LPS99 | PP10 | POPG | 1:140 | 14.4 | 15.7 | 89.2 |
| LPS100 | PP10 | POPG | 1:160 | 22.3 | 38.5 | 91.9 |
| LPS101 | PP11 | POPG | 1:40 | 9.4 | 29.7 | 64.8 |
| LPS102 | PP11 | POPG | 1:60 | 11 | 14.7 | 83.1 |
| LPS103 | PP11 | POPG | 1:80 | 13.4 | 25.1 | 83.8 |
| LPS104 | PP11 | POPG | 1:100 | 13.3 | 20.7 | 93.5 |
| LPS105 | PP11 | POPG | 1:120 | 14.6 | 20.5 | 91.5 |
| LPS106 | PP11 | POPG | 1:140 | 15.6 | 21.3 | 97.5 |
| LPS107 | PP11 | POPG | 1:160 | 17.8 | 31.1 | 97.9 |
| LPS108 | PP11 | POPG | 1:180 | 22.2 | 43.7 | 95.9 |

Particles containing PP28 (SEQ ID NO: 294) and POPG, POPC, or DMPG were then examined. Results are summarized in Table 14.

TABLE 14
Physical Properties of Discs formed with PP28

| | | | Disc | Disc % | Disc |
|---|---|---|---|---|---|
| | | PP28:Lipid | Diameter | Poly- | Intensity |
| Particle | Lipid | Ratio | (nm) | dispersity | % |
| LPS109 | POPC | 1:60 | 13.9 | 5.6 | 87 |
| LPS110 | POPC | 1:75 | 13.6 | 10.8 | 69.7 |
| LPS111 | POPC | 1:90 | 12.7 | 8.4 | 84.9 |
| LPS112 | POPC | 1:105 | 14.9 | 19.1 | 74.8 |
| LPS113 | POPC | 1:120 | 15 | 19.4 | 75 |
| LPS114 | POPC | 1:135 | 14.8 | 6.2 | 85.6 |
| LPS115 | POPC | 1:150 | 17 | 9.8 | 88.6 |
| LPS116 | POPG | 1:60 | 11.7 | 6.3 | 94.6 |
| LPS117 | POPG | 1:80 | 12.3 | 3.9 | 92.8 |
| LPS118 | POPG | 1:100 | 13.1 | 9.9 | 84.0 |
| LPS119 | POPG | 1:120 | 13.2 | 4.9 | 95.8 |
| LPS120 | POPG | 1:140 | 15.2 | 12.7 | 92.6 |
| LPS121 | POPG | 1:160 | 17.5 | 18.7 | 98.3 |
| LPS122 | POPG | 1:180 | 17.6 | 9.3 | 95.1 |
| LPS123 | DMPG | 1:50 | 8.0 | 9.4 | 68.6 |
| LPS124 | DMPG | 1:75 | 8.7 | 5.6 | 84.9 |
| LPS125 | DMPG | 1:100 | 10.3 | 2.5 | 100.0 |
| LPS126 | DMPG | 1:125 | 9.9 | 4.7 | 98.8 |
| LPS127 | DMPG | 1:150 | 11.0 | 4.5 | 94.3 |
| LPS128 | DMPG | 1:175 | 13.0 | 4.7 | 100.0 |
| LPS129 | DMPG | 1:200 | 11.6 | 4.4 | 91.0 |
| LPS130 | DMPG | 1:225 | 13.1 | 5.4 | 87.0 |

Particles containing DMPG (LPS 124-131) were found to consistently have a more uniform size distribution (i.e., lower polydispersity %), smaller hydrodynamic diameters, and fewer by-products (i.e., higher “Disc Intensity W”) than lipoprotein systems containing other lipids with a similar polypeptide:lipid ratio.

Particles including PP3 (SEQ ID NO: 296) and POPG or POP8 were then examined. Results are summarized in Table 15.

TABLE 15
Physical Properties of Discs formed with PP30

| | | | Disc | | |
|---|---|---|---|---|---|
| | | PP30:lipid | Diameter | Disc % | Disc |
| Particle | Lipid | ratio | (nm) | Polydispersity | Intensity % |
| LPS131 | POPC | 1:60 | | | |
| LPS132 | POPC | 1:75 | 8.4 | 16.4 | 68.5 |
| LPS133 | POPC | 1:90 | 8.5 | 11.9 | 81.3 |
| LPS134 | POPC | 1:105 | 9.4 | 10.5 | 77.5 |
| LPS135 | POPC | 1:120 | 10.9 | 19.6 | 67.5 |
| LPS136 | POPC | 1:135 | 11.4 | 9.7 | 81.7 |
| LPS137 | POPC | 1:150 | 12.3 | 14.6 | 82.3 |
| LPS138 | POPG | 1:70 | 10.1 | 16.4 | 78.9 |
| LPS139 | POPG | 1:90 | 10.9 | 10.9 | 81.1 |
| LPS140 | POPG | 1:110 | 12.1 | 18.0 | 80.1 |
| LPS141 | POPG | 1:130 | 18.0 | 38.6 | 93.8 |
| LPS142 | POPG | 1:150 | 33.0 | 58.8 | 97.9 |
| LPS143 | POPG | 1:170 | 37.1 | 49.2 | 93 |
| LPS144 | POPG | 1:190 | 18.1 | 27.8 | 18.7 |

Particles containing apolipoproteins PP1, PP5, PP9, PP10 or PP11, and POPS as the lipid component were then examined. The results are summarized in Table 16.

TABLE 16
Physical Properties of Discs formed
with Various Apolipoproteins and POPS

| | | Apolipo- | Disc | Disc % | Disc |
|---|---|---|---|---|---|
| | Apolipo- | protein:POPS | Diameter | Poly- | Intensity |
| Particle | protein | ratio | (nm) | dispersity | % |
| LPS145 | PP1 | 1:50 | 8.7 | 19.6 | 12.4 |
| LPS146 | PP1 | 1:70 | 11.9 | 22.4 | 73.0 |
| LPS147 | PP1 | 1:90 | 11.9 | 29.4 | 79.7 |
| LPS148 | PP1 | 1:110 | 10.4 | 11.4 | 80.4 |
| LPS149 | PP1 | 1:130 | 9.1 | 21.5 | 52.6 |
| LPS150 | PP1 | 1:150 | 9.6 | 16.6 | 69.3 |
| LPS151 | PP1 | 1:170 | 9.3 | 20.2 | 63.3 |
| LPS152 | PP5 | 1:50 | 12.3 | 23.6 | 54.9 |
| LPS153 | PP5 | 1:70 | 11.2 | 12.9 | 79.4 |
| LPS154 | PP5 | 1:90 | 10.2 | 17.6 | 72.9 |
| LPS155 | PP5 | 1:110 | 10.5 | 25.3 | 67.6 |
| LPS156 | PP5 | 1:130 | 9.3 | 15.8 | 54.1 |
| LPS157 | PP5 | 1:150 | 9.3 | 7.7 | 56.2 |
| LPS158 | PP9 | 1:50 | 10.6 | 21.0 | 37.9 |
| LPS159 | PP9 | 1:70 | 12.8 | 28.6 | 54.7 |
| LPS160 | PP9 | 1:90 | 11.6 | 28.2 | 49.9 |
| LPS161 | PP9 | 1:110 | 9.2 | 15.6 | 72.6 |
| LPS162 | PP9 | 1:130 | 9.6 | 14.9 | 56.7 |
| LPS163 | PP9 | 1:150 | 9.6 | 27.0 | 56.4 |
| LPS164 | PP10 | 1:50 | 8.0 | 12.9 | 74.9 |
| LPS165 | PP10 | 1:70 | 9.0 | 29.4 | 77.2 |
| LPS166 | PP10 | 1:90 | 8.5 | 15.0 | 61.6 |
| LPS167 | PP10 | 1:110 | 8.4 | 17.3 | 75.4 |
| LPS168 | PP10 | 1:130 | 7.9 | 10.7 | 61.4 |
| LPS169 | PP10 | 1:150 | 8.6 | 12.9 | 57.4 |
| LPS170 | PP11 | 1:50 | 8.2 | 9.8 | 81.2 |
| LPS171 | PP11 | 1:70 | 8.8 | 22.5 | 90.1 |
| LPS172 | PP11 | 1:90 | 8.1 | 14.4 | 77.8 |
| LPS173 | PP11 | 1:110 | 9.1 | 24.8 | 50.4 |
| LPS174 | PP11 | 1:130 | 8.8 | 18.4 | 55.2 |
| LPS175 | PP11 | 1:150 | 9.5 | 19.6 | 61.9 |

Compared to lipoproteins containing other lipids but with a similar polypeptide:lipid ratio, discs with POPS were found to have consistently lower hydrodynamic diameters. These observations are consistent with the data located in Table 10 described particles containing PP2. In addition, lipoproteins containing the variant apolipoproteins PP10 and PP11 (LPS 164-175) were among the smallest particles measured across all experiments.

Lipoprotein assembly conditions which were found to yield the most uniform particle populations and fewest by-products (as assessed qualitatively by native PAGE and quantitatively by DLS) were repeated at larger scale (about 1-279 mg of input purified apolipoprotein), yielding about 0.5-224 mg of reaction product (by protein mass) for select lipoprotein systems. Table 17 summarizes the physical properties of discs formed at this scale.

TABLE 17
Physical Properties of Discs formed with Various Apolipoproteins

| | | | Apolipo- | Disc | Disc % |
|---|---|---|---|---|---|
| | Apolipo- | | protein:lipid | diameter | poly- |
| Particle | protein | Lipid | ratio | (nm) | dispersity |
| LPS5 | PP2 | POPC | 1:95 | 17.8 | 14.5 |
| LPS16 | PP2 | DMPC | 1:180 | 9.0 | 5.1 |
| LPS123 | PP2 | POPG | 1:100 | 14.5 | 26.7 |
| LPS4 | PP1 | POPC | 1:110 | 20.1 | 10.3 |
| LPS6 | PP3 | POPC | 1:125 | 12.9 | 19.1 |
| LPS176 | PP5 | POPC | 1:110 | 17.0 | 13.3 |
| LPS57 | PP5 | POPG | 1:100 | 15.0 | 21.6 |
| LPS114 | PP28 | POPC | 1:135 | 17.5 | 16.1 |
| LPS177 | PP28 | POPG | 1:110 | 14.1 | 15.8 |
| LPS178 | PP28 | DMPG | 1:120 | 10.5 | 9.2 |
| LPS1 | PP9 | POPC | 1:105 | 13.9 | 5.2 |
| LPS2 | PP10 | POPC | 1:100 | 14.6 | 5.0 |
| LPS179 | PP11 | POPC | 1:75 | 13.0 | 4.9 |
| LPS180 | PP11 | DMPC | 1:160 | 9.0 | 3.6 |
| LPS92 | PP11 | 18:1 SM | 1:160 | 12.1 | 6.2 |

The size and three-dimensional geometry of the lipoprotein systems were also measured using transmission electron microscopy (TEM). The resulting images revealed particles that were of the expected discoidal or oblong geometry, and the sizes of the particles were calculated using image analysis software (ImageJ). The results of the size calculations are summarized in Table 18.

TABLE 18
Sizes of Discs Formed with Various
Apolipoproteins (measured by TEM)

| | | | Apolipo- | Mean disc | Standard |
|---|---|---|---|---|---|
| | | | protein:Lipid | diameter, | Deviation, |
| Particle | Protein | Lipid | Ratio | nm | nm |
| LPS181 | PP2 | DPPC | 1:135 | 13.1 | 2.85 |
| LPS182 | PP2 | 18:1 SM | 1:140 | 12.25 | 3.5 |
| LPS183 | PP2 | DMPG | 1:225 | 11.95 | 2.4 |
| LPS36 | PP4 | POPC | 1:100 | 11.1 | 2.5 |
| LPS57 | PP5 | POPG | 1:100 | 12.7 | 3.35 |
| LPS184 | PP5 | 18:1 SM | 1:160 | 12.35 | 2.35 |
| LPS185 | PP5 | DMPG | 1:175 | 12.3 | 2.35 |
| LPS186 | PP5 | DMPC | 1:160 | 11.45 | 2.05 |
| LPS187 | PP5 | DPPC | 1:135 | 12.4 | 2.8 |
| LPS178 | PP28 | DMPG | 1:120 | 11.8 | 1.8 |
| LPS177 | PP28 | POPG | 1:110 | 11.8 | 2.1 |
| LPS188 | PP28 | POPS | 1:180 | 10.8 | 1.6 |
| LPS189 | PP28 | DMPC | 1:200 | 12.5 | 2.6 |
| LPS190 | PP30 | POPS | 1:160 | 10.8 | 1.5 |

It was observed that the disc sizes measured by TEM and DLS were largely consistent with one another.

The binding affinities of particles containing various polypeptides and POPC or POPG for the low-density lipoprotein receptor (LDLR) were measured using SPR. The results are summarized in Table 19.

TABLE 19
Approximate binding affinities of lipoprotein systems for LDLR

| | | | | Approximate | |
|---|---|---|---|---|---|
| | | | Apolipoprotein:lipid | binding | |
| Particle | Apolipoprotein | Lipid | ratio | affinity (K_{d}) | |
| LPS5 | PP2 | POPC | 1:95 | 80 | pM |
| LPS4 | PP1 | POPC | 1:110 | 800 | pM |
| LPS114 | PP28 | POPC | 1:135 | 80 | pM |
| LPS23 | PP2 | POPG | 1:100 | 20 | nM |
| LPS177 | PP28 | POPG | 1:110 | 20 | nM |
| LPS176 | PP5 | POPC | 1:110 | 903 | pM |
| LPS57 | PP5 | POPG | 1:100 | 890 | nM |
| LPS1 | PP9 | POPC | 1:105 | 330 | pM |
| LPS2 | PP10 | POPC | 1:100 | 385 | pM |
| LPS3 | PP11 | POPC | 1:90 | 125 | pM |
| LPS6 | PP3 | POPC | 1:125 | 8.6 | nM |

The 10-fold greater affinity of LPS5 (PP2/POPC) for LDLR compared to LPS4 (PP1/POPC) and LPS176 (PP5/POPC) recapitulated the expected apolipoprotein isoform dependence, which provided confidence in the validity of the method. It was also observed that lipoproteins with POPC as the lipid component and PP2 or PP28 compared to particles containing apolipoprotein variants PP9, PP10, and PP11 had similar LDLR binding affinities (no greater than a 5-fold difference in K_{D}). It was also observed that particles formed with POPG exhibited lower binding affinities (K_{D} values about 250- to 1000-fold higher) than particles formed with POPC.

The thermal stability of particles including POPC and POPG with a variety of apolipoproteins of the present disclosure were then measured. The “onset temperature” (i.e., the temperature at which an exponential increase in particle radius was observed) is provided below in Table 20.

TABLE 20
Thermal stability of Particles

| | | | | | Onset |
|---|---|---|---|---|---|
| | | | | | Temperature |
| | | | Apolipo- | Onset | Standard |
| | Apolipo- | | protein:lipid | Temperature | Deviation |
| Particle | protein | Lipid | ratio | (° C.) | (° C.) |
| LPS4 | PP1 | POPC | 1:110 | 65.68 | 0.50 |
| LPS5 | PP2 | POPC | 1:95 | 65.65 | 1.45 |
| LPS6 | PP3 | POPC | 1:125 | 55.02 | 5.14 |
| LPS176 | PP5 | POPC | 1:110 | 69.87 | 0.40 |
| LPS114 | PP28 | POPC | 1:135 | 62.58 | 0.76 |
| LPS1 | PP9 | POPC | 1:105 | 70.14 | 2.49 |
| LPS2 | PP10 | POPC | 1:100 | 69.34 | 1.62 |
| LPS3 | PP11 | POPC | 1:90 | 71.11 | 0.07 |
| LPS23 | PP2 | POPG | 1:100 | 73.08 | 2.27 |
| LPS57 | PP5 | POPG | 1:100 | 75.10 | 4.16 |
| LPS177 | PP28 | POPG | 1:110 | 70.12 | 5.16 |
| LPS133 | PP30 | POPC | 1:90 | 65.69 | 11.15 |
| LPS140 | PP30 | POPG | 1:110 | 60.52 | 7.99 |

Particles containing POPC and PP5, PP9, PP10, and PP11 display increased stability relative to particles containing POPC and PP1, PP2, PP3, or PP30. It was also observed that the average onset temperatures of lipoproteins containing POPC are 6.7° C. lower than those of the corresponding particles with POPG. Lipoproteins containing PP30 exhibit comparable or decreased stability when compared to the other discs assessed.

The stability of exemplary lipoproteins systems to extended storage at high and low temperatures was then investigated. Particles LPS 220, 226, and 198 were synthesized using the methods described herein using PP5 and POPC, POPG, or DMPG at their optimal protein:lipid ratio. The particles were stored at either 4° C. or 37° C. for 7 days in a PBS buffer. The samples were then analyzed by DLS to measure the size, polydispersity, light scattering intensity, and mass percentage of each particle in the sample after storage. The results are summarized in Table 21.

TABLE 21
Thermal stability (high temperature) of Particles Including PP5

| | | Apolipopro- | Storage | | Disc | Disc | Disc | Disc |
|---|---|---|---|---|---|---|---|---|
| | | tein:Lipid | Temperature | | Diameter | Polydispersity | Intensity | Mass |
| Particle | Lipid | Ratio | (° c.) | | (nm) | % | % | % |
| LPS176 | POPC | 1:110 | 37° | C. | 22.35 +/− 1.05 | 20.4 +/− 10.0 | 51.2 +/− 5.5 | 97.6 +/− 0.3 |
| LPS176 | POPC | 1:110 | 4° | C. | 16.85 +/− 1.25 | 15.55 +/− 7.05 | 89.3 +/− 3.5 | 98.6 +/− 0.2 |
| LPS57 | POPG | 1:100 | 37° | C. | 14.55 +/− 0.45 | 23.4 +/− 3.8 | 94.25 +/− 2.15 | 99.25 +/− 0.35 |
| LPS57 | POPG | 1:100 | 4° | C. | 10.75 +/− 0.05 | 5 +/− 0 | 94.6 +/− 2.2 | 99.3 +/− 0.5 |
| LPS185 | DMPG | 1:175 | 37° | C. | 15.35 +/− 0.55 | 33.7 +/− 1.5 | 72.1 +/− 4.5 | 99.05 +/− 0.45 |
| LPS185 | DMPG | 1:175 | 4° | C. | 12.45 +/− 0.45 | 8.25 +/− 1.65 | 93.6 +/− 2.3 | 99.7 +/− 0.3 |

Greater decomposition (as measured by decreases in “Disc Intensity %” and “Disc Mass %” and increases in “Disc Diameter” when comparing the 4° C. and 37° C. storage conditions) was observed with the lipoprotein system containing POPC compared to those containing POPG or DMPG.

The stability of exemplary lipoprotein systems to extended cold storage and repeated freeze-thaw cycles was then investigated. Particles LPS 216-221 were synthesized using the methods described herein using POPC and various apolipoproteins at their optimal protein:lipid ratio. The particles were stored at −20° C. for up to 20 days in a PBS buffer and subjected to up to three freeze-thaw (FIT) cycles. The percentage of remaining intact lipoprotein in each sample was measured using DLS (calculated using “Disc Intensity W” compared to the value at the beginning of the experiment) at the 11- and 20-day timepoints. The results are summarized below in Table 22.

TABLE 22
Thermal stability (low temperature) of Particles Including POPC

| | | | % | % |
|---|---|---|---|---|
| | | | Remaining | Remaining |
| | | Apolipoprotein:Lipid | at 11 Days | at 20 Days |
| Particle | Apolipoprotein | Ratio | (2 f/t) | (3 f/t) |
| LPS4 | PP1 | 1:110 | 99.3 | 83.5 |
| LPS5 | PP2 | 1:95 | 97.2 | 58.0 |
| LPS6 | PP3 | 1:125 | 71.5 | 38.5 |
| LPS1 | PP9 | 1:105 | 106.3 | 99.0 |
| LPS2 | PP10 | 1:100 | 104.0 | 78.5 |
| LPS3 | PP11 | 1:90 | 95.1 | 68.9 |

After 11 days of storage at −20° C. and two freeze-thaw cycles, it was observed that lipoproteins, containing apolipoprotein variants PP9, PP10, and PP11 show comparable stability to particles with PP1 or PP2 and minimal signs of decomposition (disc remaining >95%). In contrast, particles containing PP3 show decreased stability.

After 20 days of storage at −20° C. and three freeze-thaw cycles, it was observed that lipoproteins containing apolipoprotein variants PP9, PP10, and PP11 all show improved stability compared to particles with PP2, which is the naturally occurring apolipoprotein variant that is most structurally similar.

### Conclusions

Synthetically modified variants of naturally occurring apolipoproteins readily form discoidal particles using the methods described herein. Novel exemplary apolipoproteins demonstrate the ability to form lipoproteins of comparable size, shape, and morphology relative to naturally occurring lipoproteins. The corresponding lipoprotein particles also demonstrate comparable binding to a mechanistically relevant receptor (LDLR) and increased durability in temperature-based stability challenges relative to lipoproteins containing naturally occurring polypeptides. It was also observed that both lipid and polypeptide identity can be adjusted to modulate the biophysical parameters (e.g., size, polydispersity, and thermal stability) of the resulting lipoprotein systems. Lastly, the lipoproteins described herein demonstrate increased resistance to extended cold storage and repeated freeze-thaw cycles relative to lipoproteins containing naturally occurring polypeptides.

### Example 3: Comparison of Cellular Uptake and Cholesterol Efflux of Lipoprotein Systems

### Objective

The present example is directed towards assessing the cellular uptake and cholesterol efflux of exemplary lipoprotein systems of the present invention in primary microglia. Microglial cholesterol accumulation is associated with neurodegenerative diseases including Alzheimer's disease, and reduction of intracellular cholesterol levels is predicted to be beneficial in this context. Cellular uptake of lipoproteins is hypothesized to correlate with their ability to mobilize cellular cholesterol.

### Methods

Primary rat microglia were isolated using a Neural Tissue Dissociation Kit (P) (Miltenyi, Inc.) to perform enzymatic digestion of cerebral tissue according to the manufacturer's protocol. Once a single cell suspension had been obtained, microglia were isolated on LS columns using magnetic CD11 b/c microbeads (Miltenyi, Inc) according to the manufacturer's recommendation and then cultured in medium composed of DMEM/F12 supplemented with 10% FBS (Gibco), and 50 ng/mL granulocyte-macrophage colony-stimulating factor (GM-CSF, Peprotech). The microglia were isolated from −14 week old Sprague-Dawley rats and allowed to recover for 4 days prior to use for experiments.

Lipoproteins were applied to primary microglia at 50 μg/mL in medium containing only DMEM/F12 and 1% FBS for 24 hr, then the media was collected. The cells were fixed in 4% formaldehyde and immunostained using a mouse anti-APOE primary antibody (Novus Biologicals) at a dilution of 1:200 and a donkey anti-mouse AlexaFluor 647 secondary (JacksonImmunoc) at a dilution of 1:200, then counterstained with Hoechst 33342 to identify the nuclei. Cells were imaged with a Celllnsight CX7 at 40× magnification. Twenty-five images per well were captured using equal exposure times and analyzed using HSC studio software to quantify APOE immunoreactivity. The number of spots (representative of one or more immunolabeled disc) per cell were quantified in biological triplicate. The data are shown in Table 23 below.

Lipoprotein systems of the present invention were applied to wildtype microglia at 50 μg/mL in medium containing only DMEM/F12 and 1% FBS for 24 hr then the media was collected for quantification of cholesterol mobilization by luminescent enzyme coupled assay (Cholesterol Glo™; Promega), per the manufacturer's instructions. Media from vehicle (PBS)-treated cells was used as control. This cholesterol efflux assay was run with technical duplicates or triplicates per sample for each of three independent biological replicates. The percent increase in media cholesterol concentration relative to the vehicle control was determined in each replicate, then averaged across biological replicates. The data are shown in Table 25 below. In a separate experiment, the same method was applied to quantify the relative cholesterol mobilization of lipoprotein systems including PP6 and PP28. These data are shown in Table 27 below.

Primary microglia were isolated from −13 week old human APOE3 and human APOE4 targeted replacement rats from Inotiv (formerly Envigo) and allowed to recover for 4 days prior to use for experiments. After cells had recovered, growth media was removed and replaced with DMEM/F12 media containing only 10% heat inactivated roBS (Gibco) and 250 g/mL rat myelin which had been previously isolated via Percoll gradient. Cells were incubated overnight at 370 to induce myelin loading. Cells were then washed once with DMEM/F12 media containing 1% HI—FBS. Wash media was then replaced with DMEM/F12 media containing 1% FEBS and discs at 50 μg/mL, and cells were incubated at 370 for 6 hours. Following incubation, media was removed, and cell numbers were quantified by Hoechst 33342 staining (ThermoFisher Scientific) using a 1:1000 dilution in 1×PBS. Cells were stained for 10 minutes and then imaged with a Cellnsight CX7 at 10× magnification using equal exposure times, and capturing 25 images per well. Nuclei were quantified as a proxy for cell number with HCS studio software. Hoechst solution was removed, and cells were then lysed in a solution of 1×RIPA buffer with protease inhibitor for 15 minutes at 370. Lysates were collected and analyzed for cholesterol content by luminescent enzyme-coupled assay (Cholesterol Glo™, Promega) according to manufacturer's recommendation. Calculated cholesterol concentrations were adjusted for cell number for final analysis.

### Results

TABLE 23
Uptake of Lipoprotein Systems in Rat Primary Microglia

| Treatment (50 μg/mL) | Mean Spot Count Per Cell | SD | n |
|---|---|---|---|
| Vehicle Control | 1.466 | 0.5551 | 3 |
| LPS4 | 8.313 | 4.388 | 3 |
| LPS5 | 9.463 | 5.049 | 3 |
| LPS6 | 7.097 | 3.029 | 3 |
| LPS1 | 6.83 | 2.209 | 3 |
| LPS2 | 8.583 | 4.015 | 3 |
| LPS3 | 6.188 | 2.068 | 3 |
| LPS176 | 7.098 | 3.566 | 3 |
| LPS191 (PP9/DOPC, 1:90) | 10.03 | 3.04 | 3 |
| LPS192 (PP10/DOPC, 1:115) | 7.64 | 2.936 | 3 |
| LPS193 (PP11/DOPC, 1:110) | 7.336 | 2.06 | 3 |
| LPS194 (PP5/DOPC, 1:120) | 7.689 | 1.244 | 3 |
| LPS163 | 13.15 | 2.197 | 3 |
| LPS168 | 12.89 | 0.6196 | 3 |
| LPS173 | 12.82 | 2.612 | 3 |
| LPS157 | 11.66 | 0.9916 | 3 |

TABLE 24
Statistics Table

| Dunnett's multiple | | Adjusted |
|---|---|---|
| comparisons test | Summary | P Value |
| Control vs. LPS4 | ns | 0.0562 |
| Control vs. LPS5 | * | 0.0172 |
| Control vs. LPS6 | ns | 0.1705 |
| Control vs. LPS1 | ns | 0.212 |
| Control vs. LPS2 | * | 0.043 |
| Control vs. LPS3 | ns | 0.3435 |
| Control vs. LPS176 | ns | 0.1702 |
| Control vs. LPS191 | ** | 0.0092 |
| Control vs. LPS192 | ns | 0.1062 |
| Control vs. LPS193 | ns | 0.139 |
| Control vs. LPS194 | ns | 0.1015 |
| Control vs. LPS163 | *** | 0.0003 |
| Control vs. LPS168 | *** | 0.0003 |
| Control vs. LPS173 | *** | 0.0004 |
| Control vs. LPS157 | ** | 0.0014 |

TABLE 25
Relative Cholesterol Efflux Capacity of Lipoprotein
Systems in Wildtype Rat Primary Microglia

| Treatment | Mean Cholesterol Efflux | | |
|---|---|---|---|
| (50 μg/mL) | (% of Vehicle control | SD | n |
| Vehicle Control | 100.0 | 11.3 | 3 |
| LPS4 | 123.5 | 17.8 | 3 |
| LPS5 | 127.8 | 9.5 | 3 |
| LPS6 | 116.4 | 5.8 | 3 |
| LPS1 | 119.5 | 16.6 | 3 |
| LPS2 | 125.9 | 8.6 | 3 |
| LPS3 | 123.1 | 7.8 | 3 |
| LPS176 | 125.7 | 8.3 | 3 |
| LPS191 | 136.7 | 12.8 | 3 |
| LPS192 | 135.4 | 11.7 | 3 |
| LPS193 | 129.8 | 7.1 | 3 |
| LPS194 | 128.1 | 9.4 | 3 |
| LPS163 | 136.0 | 8.7 | 3 |
| LPS168 | 144.9 | 18.8 | 3 |
| LPS173 | 136.1 | 11.6 | 3 |
| LPS157 | 161.4 | 26.1 | 3 |

TABLE 26
Statistics Table

| Dunnett's multiple | | Adjusted |
|---|---|---|
| comparisons test | Summary | P Value |
| Control vs. LPS4 | ns | 0.2593 |
| Control vs. LPS5 | ns | 0.1187 |
| Control vs. LPS6 | ns | 0.6788 |
| Control vs. LPS1 | ns | 0.471 |
| Control vs. LPS2 | ns | 0.171 |
| Control vs. LPS3 | ns | 0.2769 |
| Control vs. LPS176 | ns | 0.1752 |
| Control vs. LPS191 | * | 0.0174 |
| Control vs. LPS192 | * | 0.0239 |
| Control vs. LPS193 | ns | 0.0808 |
| Control vs. LPS194 | ns | 0.1125 |
| Control vs. LPS163 | * | 0.0207 |
| Control vs. LPS168 | ** | 0.0023 |
| Control vs. LPS173 | * | 0.0202 |
| Control vs. LPS157 | **** | <0.0001 |

TABLE 27
Relative Cholesterol Efflux of Apolipoprotein
Targeted Replacement Rat Primary Microglia

| | APOE3 TR microglia | | | APOE4 TR microglia | | |
|---|---|---|---|---|---|---|
| | Mean % | | | Mean % | | |
| Treatment | Cellular | | | Cellular | | |
| (50 μg/mL) | Cholesterol | SD | n | Cholesterol | SD | n |
| Vehicle | 100.0 | 36.4 | 12 | 100.0 | 25.7 | 10 |
| control | | | | | | |
| LPS114 | 64.4 | 21.7 | 2 | 63.7 | 6.8 | 2 |
| LPS177 | 52.4 | 21.7 | 2 | 64.5 | 10.4 | 2 |
| LPS57 | 68.7 | 5.8 | 2 | 82.8 | 0.0 | 1 |

### Conclusions

The collected images showed cellular staining in each of the cultures subjected to discPG-3T application, but not in the vehicle control. These data suggested that lipoproteins were successfully taken up into the rat primary microglia in vitro within 24 hrs of exposure. Qualitatively, PP9, PP10, and PP11 variants formulated with POPC were taken up by primary microglia to a similar extent as PP2 lipoproteins as indicated by a similar spot count per cell. Lipoproteins lipidated with POPS had the greatest cellular uptake relative to vehicle control. In primary microglia, lipoprotein particles formulated with POPC were associated with increased cholesterol in cell culture media indicative of cholesterol efflux, but this increased media cholesterol was not significantly different from that in vehicle control-treated cells. In contrast, cholesterol efflux by POPS-containing lipoproteins significantly increased the concentration of cholesterol in media relative to vehicle control. In a separate experiment, primary microglia isolated from APOE3 or APOE4 targeted replacement rats were loaded with myelin to increase intracellular cholesterol levels and facilitate robust detection of cholesterol efflux in cell lysates. Qualitatively, PP28 formulated with either POPC or POPG results in a similar reduction of intracellular cholesterol relative to vehicle control APOE3 or APOE4 targeted replacement cells, but statistical analysis was not performed. Taken together, lipoproteins containing POPS demonstrated the greatest cellular uptake and cholesterol efflux from primary microglia.

### Example 4: Preparation and Characterization of Derivatized Lipoprotein systems

### Objective

The present example is directed towards the synthesis of particles using derivatized (e.g., oxidatively coupled) apolipoprotein systems, exemplified by oxidatively coupled PP2 (hereafter, PP2-ox) and oxidatively coupled PP11 (hereafter PP11-ox). Dimeric/oxidatively coupled APOE may be of use in inhibiting amyloid fibril formation, and therefore oxidatively coupled APOE or variants thereof may be beneficial in the treatment of a variety of diseases and disorders, e.g., Alzheimer's disease. Incorporating dimeric/oxidatively coupled polypeptides into lipoprotein systems may also confer them with beneficial pharmacokinetic properties, such as increased half-life or decreased susceptibility to proteolysis.

### Methods

To generate polypeptide homodimers, aliquots of purified polypeptide were allowed to thaw at 2-8° C. Aqueous 0.5 M tris(2-carboxyethyl)phosphine (TCEP) solution was then added to the polypeptide solution (1.5 μL per 200 μg polypeptide) to reduce any disulfide bonds that formed during storage. After incubation for >30 minutes at 2-8° C., the mixture was buffer exchanged into protein storage buffer (6 M guanidinium HCl, 20 mM MOPS, pH 7.0) using a spin desalting column (ThermoFisher) following the manufacturer's instructions. Optionally, TCEP was removed via dialysis. After buffer exchange, the concentration of the polypeptide was determined by measuring the sample's absorbance at 280 nm (A280) or performing a bicinchoninic acid (BCA) assay (ThermoFisher). If necessary, the polypeptide was diluted in protein storage buffer to the appropriate working concentration for subsequent experiments.

To perform the dimerization reaction, the polypeptide solution was treated with an aqueous solution of copper(II) chloride (Acros Organics) such that 1-30 equivalents of copper(II) were added for every molecule of polypeptide (optimal ratio determined empirically for each protein). The mixture was then heated to 50° C. in a thermal cycler (Invitrogen) for 15 minutes. To remove excess copper, the resulting solution was either desalted with a spin desalting column (ThermoFisher) equilibrated with protein storage buffer or dialyzed against a volume of protein storage buffer >250-fold greater than the volume of the polypeptide dimer solution at 2-8° C. for at least two hours.

Disc assembly and characterization was performed as described herein (e.g., Example 2).

### Results

Exemplary lipoprotein systems LPS 195-213 were assembled using the polypeptide dimerization and lipoprotein assembly methods described herein. The biophysical properties of the resulting particles containing oxidatively dimerized PP11 and DMPC, POPS, or POPC are summarized in Table 28.

TABLE 28
Physical Properties of Discs formed
with Dimerized Apolipoproteins

| | | | | Disc | Disc | Disc |
|---|---|---|---|---|---|---|
| | Apolipo- | | Apolipopro- | Diam- | % | Inten- |
| Par- | protein | | tein:Lipid | eter | Polydis- | sity |
| ticle | Monomer | Lipid | ratio | (nm) | persity | % |
| LPS195 | PP11 | DMPC | 1:100 | 12.0 | 26.2 | 57.8 |
| LPS196 | PP11 | DMPC | 1:120 | 18.5 | 46.4 | 87.2 |
| LPS197 | PP11 | DMPC | 1:140 | 22.8 | 32.9 | 84.2 |
| LPS198 | PP11 | DMPC | 1:160 | 29.6 | 38.6 | 91.1 |
| LPS199 | PP11 | DMPC | 1:180 | 29.7 | 37.8 | 98.4 |
| LPS200 | PP11 | DMPC | 1:200 | 38.3 | 42.3 | 95.8 |
| LPS201 | PP11 | POPS | 1:80 | 8.9 | 30.9 | 55.3 |
| LPS202 | PP11 | POPS | 1:100 | 8.2 | 26.7 | 51.4 |
| LPS203 | PP11 | POPS | 1:120 | 7.1 | 28.3 | 47.3 |
| LPS204 | PP11 | POPS | 1:140 | 8.6 | 22.5 | 61.3 |
| LPS205 | PP11 | POPS | 1:160 | 8.8 | 38.2 | 45.9 |
| LPS206 | PP11 | POPS | 1:180 | 10.0 | 41.6 | 68.1 |
| LPS207 | PP11 | POPC | 1:40 | 13.7 | 23.5 | 75.9 |
| LPS208 | PP11 | POPC | 1:60 | 11.8 | 5.4 | 69.6 |
| LPS209 | PP11 | POPC | 1:80 | 15.7 | 21.1 | 76.0 |
| LPS210 | PP11 | POPC | 1:100 | 17.2 | 15.6 | 88.4 |
| LPS211 | PP11 | POPC | 1:120 | 18.6 | 10.5 | 82.8 |
| LPS212 | PP11 | POPC | 1:140 | 21.9 | 13.0 | 87.5 |
| LPS213 | PP11 | POPC | 1:80 | 15.7 | 21.1 | 76.0 |

Lipoprotein assembly conditions which were found to yield the most uniform particle populations and fewest by-products (as assessed qualitatively by native PAGE and quantitatively by DLS) were repeated at about 1 mg scale (mass of input purified apolipoprotein), yielding about 0.7 mg of reaction product (by protein mass) for select lipoprotein systems. Table 29 summarizes the physical properties of discs formed at this scale.

TABLE 29
Physical Properties of Discs formed with PP11-ox

| | Apolipo- | | Apolipo- | Disc | |
|---|---|---|---|---|---|
| | protein | | protein:Lipid | Diameter | Disc % |
| Particle | Monomer | Lipid | ratio | (nm) | Polydispersity |
| LPS210 | PP11 | POPC | 1:100 | 23.7 | 27.6 |
| LPS195 | PP11 | DMPC | 1:100 | 11.1 | 12.3 |

The size and three-dimensional geometry of LPS 250 (PP11-ox/DMPC) were also measured using transmission electron microscopy (TEM). The resulting images revealed particles that were of the expected discoidal geometry, and the sizes of the particles (mean diameter=11.05 nm; standard deviation=1.95 nm) were calculated using image analysis software (ImageJ) as described above. It was observed that the disc sizes measured by TEM and DLS were highly similar to one another.

### Conclusions

Oxidatively coupled apolipoprotein systems are capable of forming lipoprotein particles of comparable size and geometry to those assembled using pre-reduced apolipoproteins.

### Example 5: Comparison of Cholesterol Efflux of Oxidatively-Coupled Lipoprotein Systems

### Objective

The present example is directed towards assessing the different functional properties of exemplary oxidatively-coupled lipoprotein systems of the present invention in CCF astrocytoma cells.

### Methods

CCF human astrocytoma cells (ATCC) were maintained in RPMI-1640 (Gibco) media supplemented with 15% non-heat inactivated FBS (Gibco). Cells were passaged every 3-4 days and kept in a 37° C., 5% CO₂ incubator.

CCF cells were seeded at 20 k cells/well in a 96-well plate for 6 hours, then media was then replaced with 100 μl of 2.5 uM NBD-cholesterol (Thermo) containing media. The NBD cholesterol probe has been demonstrated to report on cellular cholesterol efflux (Song, W., Wang, W., Wang, Y., Dou, L., Chen, L., & Yan, X. (2015). Characterization of fluorescent NBD-cholesterol efflux in THP-1-derived macrophages. Molecular Medicine Reports, 12(4), 5989-5996). CCF cells were incubated in a 37° C., 5% CO₂ incubator for 18-20 hours. After incubation, cells were washed 3 times with RPMI-1640 media containing 5% lipoprotein-depleted FBS (Kalen Biomedical) and treated with 1.2-150 μg/ml of Kisbee discs for 24 hours. Following treatment, media was transferred to a new 96-well plate and cells were fixed in 4% formaldehyde for 15-20 mins. To obtain an optimal NBD-cholesterol fluorescence in media, 100 μL of absolute ethanol was added to each 100 μL media sample. NBD-cholesterol fluorescence was measured in media and fixed cells by plate reader (Molecular Devices) at Ex/Em—463/549 nm. Media and fixed cell NBD-cholesterol fluorescence were used to calculate % NBD-cholesterol efflux. Data was normalized by setting % NBD-cholesterol efflux of PBS treatment as 0 and 150 μg/ml of control disc treatment as 100. EC₅₀ values were calculated via non-linear regression in Prism (GraphPad). The results are summarized in Table 30.

### Results

TABLE 30
NBD-cholesterol efflux EC50 values

| | Reduced | | | Oxidatively-coupled | | | |
|---|---|---|---|---|---|---|---|
| | EC50 | SD | n | | EC50 | SD | n |
| LPS5 | 18 | 0.77 | 3 | LPS214 | 4.709 | 0.19 | 3 |
| | | | | (PP2- | | | |
| | | | | ox/POPC, | | | |
| | | | | 1:140) | | | |
| LPS179 | 10.78 | 0.6 | 3 | LPS210 | 9.205 | 0.62 | 3 |
| LPS181 | 3.737 | 0.2 | 3 | LPS195 | 5.041 | 0.18 | 3 |

TABLE 31
Statistics table

| Tukey's multiple comparisons test | Summary | Adjusted P Value |
|---|---|---|
| LPS5 vs. LPS215 | **** | <0.0001 |
| LPS179vs. LPS210 | * | 0.0191 |
| LPS181 vs. LPS210 | ns | 0.0596 |

### Conclusions

Oxidatively coupled apolipoprotein systems or their reduced counterparts mobilized NBD-cholesterol from CCF astrocytoma cells in a dose-dependent manner. Statistical analysis revealed that oxidized PP2 or PP11 were more potent than their reduced counterparts when lipidated with POPC. No difference in NBD-cholesterol efflux capacity was observed for oxidized versus reduced PP11 lipidated with DMPC. These data demonstrate that oxidative coupling of apolipoproteins can improve the NBD-cholesterol efflux capacity of lipoproteins formulated with POPC.

### Example 6: Comparison of Lysosomal Cholesterol Efflux of Exemplary Lipoprotein Systems in Niemann Pick Type C (NPC) Cells

### Objective

The present example is directed towards comparing the functional properties of exemplary lipoprotein systems of the present invention in NPC patient-derived fibroblasts.

### Methods

Human fibroblasts were purchased from the Coriell Institute. Control fibroblasts (GM05659) and NPC1 patient fibroblasts (GM03123) were cultured in EMEM with L-Glutamine (Quality Biological) containing 15% non-heat inactivated FBS (Gibco) and 1% Penicillin-Streptomycin (Gibco). Fibroblasts were passaged every 2 to 3 days and incubated in a 37° C., 5% CO₂ humidified incubator. PBS (pH 7.4) (Gibco) and 0.05% Trypsin-EDTA (Gibco) were used for passaging of fibroblasts.

To assess the ability of lipoprotein systems of the present disclosure to reduce lysosomal free cholesterol content, NPC1 fibroblasts were seeded into an optically clear 96-well plate (Perkin Elmer). The following day, NPC1 fibroblasts were treated with 1.3-170 μg/mL lipoproteins or 0.78-1,000 μM hydroxypropyl-β-cyclodextrin (Thermo Scientific) or methyl-β-cyclodextrin (Alfa Aesar) for 25 hours. In select experiments, the dose response range was extended to 680 μg/mL lipoproteins. Lysosomal cholesterol content was quantified based on intensity of Filipin III (Apex Bio) staining normalized to cellular density as assessed by ToPro-3 (Thermo Scientific) staining (intensity per object area). Percent maximum cholesterol efflux was calculated based on normalization to minimum filipin intensity achieved by the positive control, 1 mM hydroxypropyl-β-cyclodextrin (Thermo Scientific) or methyl-β-cyclodextrin (Alfa Aesar), which are well-established cholesterol efflux agents. Dose response curves were fit by non-linear regression in Prism (GraphPad). The EC50 values for lysosomal cholesterol efflux (intensity per object area) are reported below.

To further assess the ability of lipoprotein systems of the present disclosure to reduce free cholesterol, the free cholesterol content of fibroblasts was measured by mass spectrometry. Control and NPC1 fibroblasts were seeded into 10-cm dishes in quadruplicate. The following day, media was aspirated and replaced with fresh media, and NPC1 fibroblasts were treated +/−100 μg/mL PP28:POPG. 24 hours after disc treatment, one plate of each treatment group was harvested via trypsinization and was used to determine cellular count for that treatment group. The remaining plates were harvested in PBS. Briefly, cells were washed twice with cold LC-grade D-PBS and then collected via scraping in cold LC-grade D-PBS. Based on cellular count measurements above, volume corresponding to equal cellular amounts across treatment groups were pelleted and snap frozen for lipidomic analysis.

Lipid extraction, MS acquisition, and data pre-processing were conducted by Lipotype GmbH, as previously described. Briefly, lipids were extracted using a chloroform/methanol procedure, then sample extracts were analyzed by direct infusion on a QExactive mass spectrometer (Thermo Scientific) equipped with a TriVersa NanoMate ion source (Advion Biosciences). Samples were analyzed in both positive and negative ion modes with a resolution of Rm/z=200=280000 for MS and Rm/z=200=17500 for MSMS experiments, in a single acquisition. Both MS and MSMS data were combined to monitor cholesterol ions as ammonium adducts. Data were analyzed with in-house developed lipid identification software based on LipidXplorer (see, e.g, Herzog, R. et al., 2012. LipidXplorer: a software for consensual cross-platform lipidomics. PloS ONE, 7(1), p.e29851, the entirety of which is incorporated herein by reference). Data post-processing and normalization were performed using an in-house developed data management system.

### Results

Efflux EC50 values of exemplary lipoprotein systems are summarized in Tables 32-34. The reported values in Table 32 were normalized to methyl-β-cyclodextrin. The values reported in Table 33 and Table 34 were normalized relative to hydroxypropyl-β-cyclodextrin.

TABLE 32
NPC lysosomal cholesterol efflux EC50
values for exemplary lipoproteins

| | EC50 (μg/mL) | R2 (intensity |
|---|---|---|
| | (intensity per | per |
| Disc Treatment | object area) | object area) |
| LPS185 | 7.756 | 0.9518 |
| LPS57 | 12.13 | 0.9306 |
| LPS126 | 16.12 | 0.9838 |
| LPS113 | 19.25 | 0.9426 |
| LPS215 (PP11:OSM, 1:160) | 23.35 | 0.9832 |
| LPS216 (PP5:DOPC, 1:120) | 26.46 | 0.9412 |
| LPS36 | 29.8 | 0.9783 |
| LPS5 | 30.37 | 0.9747 |
| LPS217 (PP11:DPPC, 1:135) | 32.67 | 0.8182 |
| LPS218 (PP5:OSM, 1:160) | 32.78 | 0.9589 |
| LPS6 | 34.61 | 0.9591 |
| LPS179 | 36.54 | 0.962 |
| LPS219 (PP2:OSM, 1:140) | 38.44 | 0.9502 |
| LPS220 (PP2:POPC/POPS, 1:140) | 41.85 | 0.9471 |
| LPS188 | 43.66 | 0.9408 |
| LPS221 (PP11:POPC, 1:100) | 43.71 | 0.8936 |
| LPS32 | 49.94 | 0.9599 |
| LPS214 | 55.05 | 0.9221 |
| LPS4 | 64.00 | 0.8813 |
| LPS222 (PP3:DMPC, 1:150) | 74.19 | 0.8695 |
| LPS176 | 75.03 | 0.9485 |
| LPS190 | 78.94 | 0.8885 |
| LPS155 | 81.81 | 0.9193 |
| LPS16 | 84.79 | 0.7415 |
| LPS223(PP3:POPS, 1:110) | 131.5 | 0.8277 |
| LPS157 | 135.9 | 0.9718 |
| LPS133 | 153.6 | 0.9298 |
| LPS157 | 378.9 | 0.8905 |
| LPS224 (PP3:DPPC, 1:150) | 394.6 | 0.7647 |
| LPS186 | 1173 | 0.8928 |
| LPS180 | 2099 | 0.4178 |
| LPS195 | 3819 | 0.7254 |
| LPS182 | Unstable | 0.8218 |
| LPS187 | Unstable | 0.7703 |
| LPS189 | Unstable | 0.7087 |

TABLE 33
NPC lysosomal cholesterol efflux EC50
values for exemplary lipoproteins

| Disc | EC50 (μg/mL) (intensity | R2 (intensity per |
|---|---|---|
| Treatment | per object area) | object area) |
| LPS23 | 7.96 | 0.96 |
| LPS177 | 8.972 | 0.94 |
| LPS114 | 9.93 | 0.95 |
| LPS5 | 10.00 | 0.95 |
| LPS36 | 24.83 | 0.95 |
| LPS133 | 81.47 | 0.85 |

TABLE 34
NPC lysosomal cholesterol efflux EC50 for LPS177 EC50

| | HPbCD | LPS178 |
|---|---|---|
| Mean (μM) | 20.45 | 0.2559 |
| R2 | 0.97 | 0.95 |
| SD | 1.43 | 0.04 |
| n | 3 | 3 |

| | |
|---|---|
| p value | 0.0017 |
| Statistical | Unpaired parametric t-test |
| test | with Welch's correction |

Table 35 presents the free cholesterol content of fibroblasts as quantified by mass spectrometry.

TABLE 35
Free Cholesterol Content of Fibroblasts

| | Free cholesterol | | |
|---|---|---|---|
| | Relative abundance | SD | n |
| Control fibroblasts, untreated | 1.00 | 0.064 | 3 |
| NPC1 fibroblasts, untreated | 2.08 | 0.038 | 3 |
| NPC1 fibroblasts + LPS177 | 1.25 | 0.020 | 3 |

### Conclusions

Novel exemplary lipoproteins reduced lysosomal cholesterol accumulation in NPC1 patient-derived fibroblasts as demonstrated by reduced filipin staining. The lipoproteins with the highest potency for reducing lysosomal cholesterol this context were those formulated with phosphatidylglycerol-containing headgroups, exemplified by POPG. Of the lipoproteins formulated with POPC, LPS114 had the highest potency of the apolipoproteins tested. LPS177 was found to be significantly more potent for reducing lysosomal cholesterol relative to hydroxypropyl-β-cyclodextrin, a well-established cholesterol efflux agent with clinical relevance to NPC disease. The reduction of intracellular free cholesterol by LPS177 lipoprotein treatment was confirmed with mass spectrometry. Taken together, these data suggest that LPS177 may be beneficial in reducing lysosomal cholesterol in the context of NPC disease.

### Example 7: Design and Evaluation of Modified Lipoprotein Systems

### Objective

The present example demonstrates the modification of lipoprotein systems of the present disclosure. This example is directed towards biotinylation as an exemplary modification.

### Methods

To achieve biotinylation of the apolipoprotein, the purified protein species was reduced and buffer exchanged into protein storage buffer (6 M guanidinium HCl, 20 mM MOPS, pH 7.0) using the methods described herein (e.g., Example 2). Subsequently, the sample was diluted to a protein concentration of 1.5 mg/mL in protein storage buffer and treated with a DMSO solution of an amine-reactive biotinylation reagent (ThermoFisher) for 1 hour at ambient temperature. The mixture was then exchanged back into protein storage buffer using a spin desalting column (ThermoFisher) to remove excess biotinylation reagent, reaction by-products, and unwanted buffers or solvents. The concentration of the biotinylated polypeptide in the resulting solution was determined by measuring the sample's absorbance at 280 nm (A280) or performing a bicinchoninic acid (BCA) assay (ThermoFisher). The polypeptide solution was then used as an input for lipoprotein assembly experiments using the methods described herein.

To achieve biotinylation of assembled lipoprotein systems, an amine-reactive biotinylation reagent (ThermoFisher) was dissolved in the appropriate solvent (DMSO or water) to generate a 50 mM solution. The biotinylation reagent solution was then combined with a solution of the lipoprotein in PBS (2 mM KH₂PO₄, 10 mM Na₂HPO₄, 140 mM NaCl, 3 mM KCl, pH 7.4) in a well-defined molar ratio of labeling reagent to protein, and the resulting mixture was allowed to incubate at 4° C. for 1-2 hours. The labeling reaction was then quenched with quenching buffer (1 M Tris-HCl, pH 7.4), the volume of which was equal to 10% of the total reaction volume. After 10 minutes, the mixture was transferred to a dialysis device (ThermoFisher) and dialyzed against 40-fold excess PBS at 4° C. to remove quenched biotinylation reagent, reaction by-products, and unwanted buffers or solvents. After 2 hours, the dialysis device was moved to fresh PBS (40-fold excess volume) and allowed to dialyze for an additional 2 hours at 4° C.

### Results

Biotinylated lipoprotein systems were characterized via native PAGE, DLS, and A280 analysis using the methods described herein. The biotin content of the samples was measured using a colorimetric plate reader assay (ThermoFisher). The extent of protein biotinylation was also quantified via peptide mapping analysis. Briefly, the protein component of the lipoprotein system was separated from the lipids by dissolving the sample in methanol and methyl tert-butyl ether (MTBE) and removing the organic phase after centrifugation. The isolated protein was then digested overnight under denaturing conditions at 37° C., reduced, acidified, and analyzed via LC-MS on an ESI-QToF (Agilent) mass spectrometer. Summing the biotinylation percentages for each peptide yielded an estimate of the number of biotin molecules per protein molecule.

To determine if biotinylation altered the size and size distribution of lipoprotein particles, an exemplary lipoprotein system containing LPS114 was synthesized and treated with varying amounts of two different biotinylation reagents: EZ-Link NHS-Biotin (ThermoFisher cat. #20217) and EZ-Link Sulfo-NHS-Biotin (ThermoFisher cat. #A39256). The samples were measured via DLS and compared to a sample of LPS115 that was subjected to the biotinylation procedure but without any biotinylation reagent in the reaction mixture. The results are summarized in Table 36.

TABLE 36
Physical Properties of Biotinylated LPS114

| | | Biotiny- | | | | |
|---|---|---|---|---|---|---|
| | Biotiny- | lation | Disc | Disc % | Disc | Disc |
| Par- | lation | Reagent | Diameter | Polydis- | Intensity | Mass |
| ticle | Reagent | Equivs. | (nm) | persity | % | % |
| LPS225 | NHS-Biotin | 0 | 18.7 | 33.5 | 89.8 | 97.4 |
| LPS226 | NHS-Biotin | 10 | 18.6 | 22.2 | 91.5 | 98.7 |
| LPS227 | NHS-Biotin | 15 | 19.8 | 25.9 | 90.7 | 98.7 |
| LPS228 | NHS-Biotin | 20 | 18.8 | 23 | 91.9 | 98.8 |
| LPS229 | NHS-Biotin | 25 | 18.2 | 17.1 | 88.2 | 97.1 |
| LPS230 | Sulfo-NHS- | 0 | 14.7 | 25.1 | 93.5 | 99.6 |
| | Biotin | | | | | |
| LPS231 | Sulfo-NHS- | 10 | 16.1 | 5 | 98.1 | 99.3 |
| | Biotin | | | | | |
| LPS232 | Sulfo-NHS- | 15 | 19.9 | 39.3 | 98.1 | 99.6 |
| | Biotin | | | | | |
| LPS233 | Sulfo-NHS- | 20 | 17.4 | 14 | 96.4 | 99.8 |
| | Biotin | | | | | |
| LPS234 | Sulfo-NHS- | 25 | 14.7 | 14 | 90.5 | 99.9 |
| | Biotin | | | | | |

It was observed that biotinylation had minimal impact on the lipoprotein sample's biophysical properties regardless of the biotinylation reagent identity and number of equivalents added to the reaction mixture.

An exemplary system containing LPS177 was then examined. As above, aliquots of LPS177 were subjected to various biotinylation conditions. In this dataset, the biotinylated particles were compared to the parent batch that did not undergo the biotinylation procedure. The results are summarized in Table 37.

TABLE 37
Physical Properties of Biotinylated Lipoproteins with LPS178

| | | Biotiny- | | | | |
|---|---|---|---|---|---|---|
| | Biotiny- | lation | Disc | Disc % | Disc | Disc |
| Par- | lation | Reagent | Diameter | Polydis- | Intensity | Mass |
| ticle | Reagent | Equivs. | (nm) | persity | % | % |
| LPS235 | — | N/A | 9.7 | 5 | 100 | 100 |
| LPS236 | NHS-Biotin | 5 | 14.0 | 16.5 | 52.1 | 99.1 |
| LPS237 | NHS-Biotin | 15 | 13.5 | 15.5 | 92.0 | 99.9 |
| LPS238 | NHS-Biotin | 25 | 14.9 | 25.5 | 88.6 | 99.8 |
| LPS239 | Sulfo-NHS- | 5 | 13.5 | 15.5 | 94.4 | 100 |
| | Biotin | | | | | |
| LPS240 | Sulfo-NHS- | 15 | 12.8 | 8.2 | 91.3 | 99.9 |
| | Biotin | | | | | |
| LPS241 | Sulfo-NHS- | 25 | 10.9 | 7.9 | 76.4 | 99.1 |
| | Biotin | | | | | |

It was observed that the biotinylation process resulted in a small increase in both disc diameter and polydispersity. The use of sulfo-NHS-biotin minimized this effect, and all samples showed minimal decomposition and by-product formation, as evidenced by the very small reductions in “Disc Mass W” (<1 percentage point in all samples).

To quantify the amount of biotin that had been conjugated to each molecule of polypeptide, select biotinylated samples were subjected to a colorimetric biotin quantification assay. The results are summarized in Table 38.

TABLE 38
Biotin quantification results via colorimetric assay

| | | | Measured Biotin |
|---|---|---|---|
| | Biotinylation | Biotinylation Reagent | Equivs. in |
| Particle | Reagent | Equivs. in Reaction | Product |
| LPS236 | NHS-Biotin | 5 | 2.04 |
| LPS237 | NHS-Biotin | 15 | 6.45 |
| LPS238 | NHS-Biotin | 25 | 7.88 |
| LPS239 | Sulfo-NHS-Biotin | 5 | 1.95 |
| LPS240 | Sulfo-NHS-Biotin | 15 | 4.88 |
| LPS241 | Sulfo-NHS-Biotin | 25 | 4.82 |
| LPS232 | Sulfo-NHS-Biotin | 15 | 4.40 |

As expected, it was observed that increasing the number of biotinylation reagent equivalents in the biotinylation reaction increased the amount of biotin per polypeptide molecule that was measured in the reaction product. This relationship appeared to plateau upon treatment with 25 equivalents of biotinylation reagent, which suggests that the polypeptide could be approaching saturation under these reaction conditions.

To confirm biotinylation conjugation to the polypeptide molecule and obtain an orthogonal measurement of the biotin:protein ratio, select biotinylation samples were subjected to peptide mapping analysis. The results are summarized in Table 39.

TABLE 39
Biotin quantification results via peptide mapping

| | | | Measured Biotin |
|---|---|---|---|
| | Biotinylation | Biotinylation Reagent | Equivs. in |
| Particle | Reagent | Equivs. in Reaction | Product |
| LPS237 | NHS-Biotin | 15 | 3.38 |
| LPS238 | NHS-Biotin | 25 | 3.88 |
| LPS240 | Sulfo-NHS-Biotin | 15 | 3.59 |
| LPS241 | Sulfo-NHS-Biotin | 25 | 3.56 |
| LPS232 | Sulfo-NHS-Biotin | 15 | 3.75 |

It was observed that there was good overall concordance between the peptide mapping data and the colorimetric assay data summarized in Table 39, especially for samples labeled with Sulfo-NHS-Biotin.

A microglia spot count analysis, comparing biotinylated and non-biotinylated particles was then performed. Primary wildtype rat microglia were isolated and subcultured as described above. A matched set of lipoproteins, non-biotinylated versus biotinylated pre-assembly, were applied for 2 hours at 10 μg/mL. Finally, spot count per cell was determined as described above. This experiment was performed in biological singlicate and technical triplicates. The experiment was performed comparing LPS243 (including a 1:120 ratio of PP2 to POPC) and LPS244 (including a 1:60 ratio of biotinylated PP2 to POPC and a 1:15 preassembly ratio of PP2 to biotin).

TABLE 40
Spot Count Per Cell for LPS242 and LPS243

| Treatment | Mean | SD | n |
|---|---|---|---|
| Vehicle control | 1.36 | 0.4521 | 3 |
| LPS242 | 3.453 | 0.7805 | 3 |
| LPS243 | 3.637 | 0.4438 | 3 |

TABLE 41
Statistics

| Tukey's multiple comparisons test | Summary | Adjusted P Value |
|---|---|---|
| Control vs. 241 | * | 0.0106 |
| Control vs. LPS242 | ** | 0.0071 |
| LPS241 vs LPS242 | ns | 0.9219 |

NPC cholesterol efflux was then determined. NPC fibroblasts were subcultured as described above, and the filipin-based lysosomal cholesterol efflux assay was run as described herein. The experiments were performed using LPS178 (including a 1:110 ratio of PP28 to POPG) to LPS240 (including a 1:110 ratio of PP28 to POPG biotinylated with 5 equivs. sulfo-NHS-biotin post-assembly). Results are summarized in Tables 42 and 43.

TABLE 42
EC 50 values for LPS177 and LPS239 in NPC Cholesterol Efflux

| Treatment | Mean EC50 | SD | n |
|---|---|---|---|
| LPS177 | 9.42 | 1.28 | 3 |
| LPS239 | 7.36 | 1.77 | 3 |

TABLE 43
Statistics

| Unpaired t test with Welch's correction | Summary | P value |
|---|---|---|
| LPS177 vs LPS239 | ns | 0.148 |

RedC12 efflux data was then generated comparing non-biotinylated and biotinylated derivates. APOE knockout H4 astroglioma cells were subcultured as described herein, and the RedC12 whole cell lipid efflux assay was run as described above using LPS177 and LPS239. The results are summarized in Tables 44 and 45.

TABLE 44
EC 50 values for LPS178 and LPS240 in RedC12 Efflux

| Treatment | Mean EC50 | SD | n |
|---|---|---|---|
| LPS177 | 19.32 | 1.047 | 2 |
| LPS239 | 24.14 | 4.723 | 2 |

TABLE 45
Statistics

| Unpaired t test with Welch's correction | Summary | P value |
|---|---|---|
| LPS177 vs. LPS239 | ns | 0.3775 |

### Conclusion

Both LPS242 and biotinylated LPS243 lipoproteins were taken up by primary microglia as previously observed. Statistical analyses revealed that there were no differences in spot counts per cell for LPS242 and LPS243 in this biological singlicate experiment, suggesting that biotin labeling does not impact cellular uptake. LPS177 and biotinylated LPS239 mobilized lysosomal cholesterol from NPC patient fibroblasts as demonstrated by reduced filipin staining. Statistical analysis revealed that there was no significant difference in the EC50 values for LPS177 and biotinylated LPS239, suggesting that biotin labeling does not impact lysosomal cholesterol efflux.

Further, both LPS177 and LPS239 mobilized lipids from cells as demonstrated by efflux of RedC12-labeled lipids. Statistical analysis revealed that there was no significant difference in the EC50 for whole cell lipid efflux for LPS177 and LPS239, suggesting that biotin labeling does not impact lipid efflux.

### Example 8: Design and Evaluation of a Further Series of Lipoprotein Systems

### Objective

The present example is directed toward the synthesis and characterization of exemplary lipoprotein systems of the present invention.

### Methods

To assess the ability of lipid-free apolipoproteins to remodel DMPC liposomes, aliquots of purified polypeptide were allowed to thaw at 2-8° C. Aqueous 0.5 M tris(2-carboxyethyl)phosphine (TCEP) solution was then added to the polypeptide solution (1.5 μL per 200 μg polypeptide) to reduce any disulfide bonds that formed during storage. After incubation for >30 minutes at 2-8° C., the mixture was buffer exchanged into PBS (2 mM KH₂PO₄, 10 mM Na₂HPO₄, 140 mM NaCl, 3 mM KCl, pH 7.4) using a spin desalting column (ThermoFisher) following the manufacturer's instructions. After buffer exchange, the concentration of the polypeptide was determined by measuring the sample's absorbance at 280 nm (A280). In parallel, a mixture of DMPC (1,2-dimyristoyl-sn-glycero-3-phosphocholine) in detergent-free lipid buffer (20 mM Tris HCl, 100 mM NaCl, pH 7.4) was prepared from neat DMPC powder and adjusted to about 10 mM.

The polypeptide and DMPC solutions were combined in technical duplicate or triplicate in a UV-transparent 96-well plate (Corning) such that the molar ratio of protein:lipid was 1:75. The absorbance of the wells at 325 nm was measured every minute for 90 minutes. The rate of DMPC clearance (Kₐ) was calculated by fitting an exponential decay rate model to the time series data that was normalized to the absorbance value at the beginning of the experiment (A325 at T=0 min was set equal to 1.00).

### Results

Polypeptides PP124 (SEQ ID NO: 876), PP125 (SEQ ID NO: 877), PP126 (SEQ ID NO: 878), PP127 (SEQ ID NO: 879), PP128 (SEQ ID NO: 880), and PP129 (SEQ ID NO: 881) were expressed, harvested, and purified using the methods described herein (see, e.g., Example 1). The ability of these novel apolipoprotein variants to interact with and remodel DMPC liposomes was assessed to serve as a simplified proxy of the disc assembly process. The rate at which apolipoproteins achieve such remodeling is related to the facility with which they form the lipoprotein systems described in the present invention. The data are summarized in Table 46.

TABLE 46
Rates of DMPC clearance for various apolipoproteins,
including novel variants

| Protein | Kₐ (s⁻¹) |
|---|---|
| PP1 | 0.0032 |
| PP2 | 0.0145 |
| PP5 | 0.0128 |
| PP28 | 0.1085 |
| PP124 | 0.0221 |
| PP125 | 0.0247 |
| PP126 | 0.0383 |
| PP127 | 0.0058 |
| PP128 | 0.0149 |
| PP129 | 0.0068 |

It was observed that PP28 cleared DMPC liposomes most quickly, followed by PP126, PP125, PP124, and PP128. All novel variants exhibited greater activity in this assay than the naturally occurring apolipoprotein PP1.

Building on the DMPC clearance results, particles including PP124 and POPC, POPG, DMPC, or POPS were assembled using the methods described herein. Results are summarized in Table 47.

TABLE 47
Physical Properties of Discs formed with PP124

| | | | Disc | | |
|---|---|---|---|---|---|
| | | PP124:Lipid | Diameter | Disc % | |
| Particle | Lipid | ratio | (nm) | Polydispersity | Disc Intensity % |
| LPS244 | POPC | 1:62 | 11.7 | 24.4 | 79.3 |
| LPS245 | POPC | 1:77 | 15.3 | 7.6 | 96.5 |
| LPS246 | POPC | 1:91 | 16.7 | 8.3 | 90.1 |
| LPS247 | POPC | 1:106 | 15.5 | 7.2 | 90.7 |
| LPS248 | POPC | 1:121 | 16.4 | 7.4 | 94.9 |
| LPS249 | POPC | 1:135 | 19.4 | 16.4 | 98.5 |
| LPS250 | POPC | 1:149 | 19.4 | 17.1 | 100 |
| LPS251 | DMPC | 1:58 | 11.2 | 2.8 | 91.8 |
| LPS252 | DMPC | 1:77 | 12.1 | 11.2 | 90.7 |
| LPS253 | DMPC | 1:96 | 13.3 | 14.1 | 100 |
| LPS254 | DMPC | 1:135 | 12.7 | 9.2 | 100 |
| LPS255 | DMPC | 1:155 | 14.3 | 10.5 | 100 |
| LPS256 | DMPC | 1:174 | 15.4 | 22 | 100 |
| LPS257 | POPG | 1:58 | 11.2 | 7.3 | 93.7 |
| LPS258 | POPG | 1:78 | 13.0 | 5.6 | 89.7 |
| LPS259 | POPG | 1:98 | 13.6 | 3.9 | 96.7 |
| LPS260 | POPG | 1:118 | 13.6 | 5.2 | 96 |
| LPS261 | POPG | 1:138 | 15.3 | 15.6 | 94.9 |
| LPS262 | POPG | 1:158 | 16.2 | 9.4 | 95.9 |
| LPS263 | POPG | 1:178 | 15.2 | 3.2 | 95.2 |
| LPS264 | POPS | 1:58 | 10.6 | 23.4 | 66.3 |
| LPS265 | POPS | 1:78 | 12.5 | 3.3 | 90.4 |
| LPS266 | POPS | 1:98 | 13.7 | 9 | 89.2 |
| LPS267 | POPS | 1:118 | 14.2 | 6.7 | 90.8 |
| LPS268 | POPS | 1:138 | 16 | 10.4 | 92 |
| LPS269 | POPS | 1:158 | 20.4 | 17 | 90.8 |
| LPS270 | POPS | 1:178 | 19 | 19.6 | 94.8 |

Particles including PP125 and POPC, POPG, DMPC, or POPS were then examined. Results are summarized in Table 48.

TABLE 48
Physical Properties of Discs formed with PP125

| | | | Disc | | |
|---|---|---|---|---|---|
| | | PP125:lipid | Diameter | Disc % | |
| Particle | Lipid | ratio | (nm) | Polydispersity | Disc Intensity % |
| LPS271 | POPC | 1:60 | 11.6 | 4.4 | 90.9 |
| LPS272 | POPC | 1:80 | 12.7 | 8.4 | 89.3 |
| LPS273 | POPC | 1:100 | 13 | 10.4 | 90.5 |
| LPS274 | POPC | 1:120 | 13.4 | 7.3 | 91.2 |
| LPS275 | POPC | 1:140 | 14.7 | 9.6 | 85.3 |
| LPS276 | POPC | 1:160 | 15.0 | 13.6 | 94.0 |
| LPS277 | POPC | 1:180 | 17.7 | 21.6 | 91.5 |
| LPS278 | DMPC | 1:60 | 12.3 | 12.5 | 22.1 |
| LPS279 | DMPC | 1:80 | 12.8 | 13.8 | 26.0 |
| LPS280 | DMPC | 1:100 | 16.1 | 19.5 | 31.3 |
| LPS281 | DMPC | 1:120 | 14.9 | 14.5 | 37.8 |
| LPS282 | DMPC | 1:140 | 21.2 | 37.2 | 53.0 |
| LPS283 | DMPC | 1:180 | 14.5 | 10.6 | 12.4 |
| LPS284 | DMPC | 1:60 | 12.3 | 12.5 | 22.1 |
| LPS285 | POPG | 1:60 | 12.7 | 12.6 | 86.4 |
| LPS286 | POPG | 1:80 | 13.9 | 20.8 | 88.3 |
| LPS287 | POPG | 1:100 | 14.1 | 4.2 | 92.8 |
| LPS288 | POPG | 1:120 | 15.2 | 16.3 | 78.3 |
| LPS289 | POPG | 1:140 | 14.5 | 5.3 | 88.9 |
| LPS290 | POPG | 1:160 | 17.0 | 18.7 | 92.4 |
| LPS291 | POPG | 1:180 | 19.0 | 21.7 | 93.5 |
| LPS292 | POPS | 1:60 | 9.7 | 12.1 | 85 |
| LPS293 | POPS | 1:80 | 12.3 | 11.2 | 84.9 |
| LPS294 | POPS | 1:100 | 12.4 | 7.2 | 88.2 |
| LPS295 | POPS | 1:120 | 15.6 | 22.6 | 80.1 |
| LPS296 | POPS | 1:140 | 20 | 32.7 | 94 |
| LPS297 | POPS | 1:160 | 28.8 | 41 | 97.5 |
| LPS298 | POPS | 1:180 | 22.8 | 29.5 | 97.6 |

Particles including PP126 and POPC, POPG, DMPC, or POPS were then examined. Results are summarized in Table 49.

TABLE 49
Physical Properties of Discs formed with PP126

| | | | Disc | | |
|---|---|---|---|---|---|
| | | PP126:lipid | diameter | Disc % | |
| Particle | Lipid | ratio | (nm) | polydispersity | Disc intensity % |
| LPS299 | POPC | 1:55 | 14.1 | 6.7 | 95.8 |
| LPS300 | POPC | 1:70 | 14.8 | 4.9 | 92.5 |
| LPS301 | POPC | 1:85 | 17.8 | 9.7 | 100 |
| LPS302 | POPC | 1:100 | 18.9 | 14.5 | 100 |
| LPS303 | POPC | 1:115 | 18.1 | 6.5 | 100 |
| LPS304 | POPC | 1:130 | 18 | 5.9 | 100 |
| LPS305 | POPC | 1:145 | 21.6 | 19.7 | 100 |
| LPS306 | DMPC | 1:60 | 9.8 | 5 | 92.4 |
| LPS307 | DMPC | 1:80 | 12 | 5 | 97.4 |
| LPS308 | DMPC | 1:100 | 10.3 | 2.2 | 84.9 |
| LPS309 | DMPC | 1:120 | 17.2 | 52.6 | 100 |
| LPS310 | DMPC | 1:140 | 15.7 | 14.6 | 98.9 |
| LPS311 | DMPC | 1:160 | 20 | 35.8 | 100 |
| LPS312 | DMPC | 1:180 | 17.5 | 12.4 | 97.8 |
| LPS313 | POPG | 1:60 | 10.9 | 21.6 | 73.4 |
| LPS314 | POPG | 1:80 | 11.9 | 11.1 | 85.1 |
| LPS315 | POPG | 1:100 | 12.9 | 9.4 | 92.3 |
| LPS316 | POPG | 1:140 | 19.4 | 18.5 | 91.0 |
| LPS317 | POPG | 1:160 | 22.8 | 34.1 | 95.7 |
| LPS318 | POPG | 1:180 | 23.8 | 25.2 | 93.3 |
| LPS319 | POPG | 1:60 | 10.9 | 21.6 | 73.4 |
| LPS320 | POPS | 1:60 | 10.5 | 22 | 73 |
| LPS321 | POPS | 1:80 | 12.5 | 24.9 | 77.2 |
| LPS322 | POPS | 1:100 | 13.6 | 18.5 | 80.7 |
| LPS323 | POPS | 1:120 | 17.3 | 19.6 | 91.5 |
| LPS324 | POPS | 1:140 | 20.2 | 30.5 | 76.8 |
| LPS325 | POPS | 1:160 | 24.2 | 40.2 | 92.5 |
| LPS326 | POPS | 1:180 | 31 | 51.2 | 97.2 |

Particles including PP127 and POPG, DMPC, or POPS were then examined. Results are summarized in Table 50.

TABLE 50
Physical Properties of Discs formed with PP127

| | | | Disc | | |
|---|---|---|---|---|---|
| | | PP127:Lipid | Diameter | Disc % | |
| Particle | Lipid | Ratio | (nm) | Polydispersity | Disc Intensity % |
| LPS327 | DMPC | 1:60 | 12.3 | 6.8 | 100 |
| LPS328 | DMPC | 1:80 | 13.8 | 20.9 | 100 |
| LPS329 | DMPC | 1:100 | 15.5 | 30.2 | 100 |
| LPS330 | DMPC | 1:120 | 17.2 | 26.7 | 100 |
| LPS331 | DMPC | 1:140 | 15.7 | 7.6 | 100 |
| LPS332 | DMPC | 1:160 | 18.7 | 21.7 | 100 |
| LPS333 | DMPC | 1:180 | 22.7 | 36.3 | 100 |
| LPS334 | POPG | 1:60 | 9.2 | 14.1 | 81.6 |
| LPS335 | POPG | 1:80 | 11.0 | 14.4 | 77.4 |
| LPS336 | POPG | 1:100 | 12.9 | 11.6 | 87.2 |
| LPS337 | POPG | 1:120 | 15.0 | 6.3 | 79.7 |
| LPS338 | POPG | 1:140 | 16.8 | 15.6 | 83.5 |
| LPS339 | POPG | 1:160 | 17.9 | 26.3 | 65.5 |
| LPS340 | POPG | 1:180 | 19.5 | 12.2 | 83.5 |
| LPS341 | POPS | 1:60 | 12.2 | 49 | 57.4 |
| LPS342 | POPS | 1:80 | 11.5 | 23.6 | 82.4 |
| LPS343 | POPS | 1:100 | 13 | 10.3 | 85.5 |
| LPS344 | POPS | 1:120 | 15.8 | 23.4 | 82.3 |
| LPS345 | POPS | 1:140 | 21.2 | 48.8 | 75.7 |
| LPS346 | POPS | 1:160 | 19.5 | 30.9 | 67.8 |
| LPS347 | POPS | 1:180 | 21.2 | 27.5 | 80.5 |

Particles including PP128 and POPG, DMPC, or POPS were then examined. Results are summarized in Table 51.

TABLE 51
Physical Properties of Discs formed with PP128

| | | | Disc | | |
|---|---|---|---|---|---|
| | | PP128:Lipid | Diameter | Disc % | |
| Particle | Lipid | ratio | (nm) | Polydispersity | Disc Intensity % |
| LPS348 | DMPC | 1:53 | 11.0 | 9.0 | 58.8 |
| LPS349 | DMPC | 1:70 | 11.5 | 20.6 | 71.7 |
| LPS350 | DMPC | 1:88 | 9.9 | 9 | 58.3 |
| LPS351 | DMPC | 1:105 | 12.8 | 16.3 | 78.6 |
| LPS352 | DMPC | 1:123 | 11.6 | 13.7 | 63.5 |
| LPS353 | DMPC | 1:141 | 11.7 | 7.1 | 70.6 |
| LPS354 | DMPC | 1:158 | 13.7 | 10.2 | 88.4 |
| LPS355 | POPG | 1:49 | 8.9 | 25.6 | 64.7 |
| LPS356 | POPG | 1:69 | 10.6 | 21.2 | 71.3 |
| LPS357 | POPG | 1:89 | 11.1 | 11.2 | 76 |
| LPS358 | POPG | 1:109 | 15.7 | 12 | 22.7 |
| LPS359 | POPG | 1:129 | 12.1 | 15.2 | 10.6 |
| LPS360 | POPG | 1:149 | 17.3 | 11.4 | 18.7 |
| LPS361 | POPG | 1:49 | 8.9 | 25.6 | 64.7 |
| LPS362 | POPS | 1:60 | 12.2 | 49 | 57.4 |
| LPS363 | POPS | 1:80 | 11.5 | 23.6 | 82.4 |
| LPS364 | POPS | 1:100 | 13 | 10.3 | 85.5 |
| LPS365 | POPS | 1:120 | 15.8 | 23.4 | 82.3 |
| LPS366 | POPS | 1:140 | 21.2 | 48.8 | 75.7 |
| LPS367 | POPS | 1:160 | 19.5 | 30.9 | 67.8 |
| LPS368 | POPS | 1:180 | 21.2 | 27.5 | 80.5 |

Particles including PP129 and POPG, DMPC, or POPS were then examined. Results are summarized in Table 52.

TABLE 52
Physical Properties of Discs formed with PP129

| | | | Disc | | |
|---|---|---|---|---|---|
| | | PP129:Lipid | Diameter | Disc % | |
| Particle | Lipid | ratio | (nm) | Polydispersity | Disc Intensity % |
| LPS369 | DMPC | 1:60 | 13.8 | 9.1 | 76.8 |
| LPS370 | DMPC | 1:80 | 16.5 | 18 | 100 |
| LPS371 | DMPC | 1:100 | 17.1 | 24.7 | 100 |
| LPS372 | DMPC | 1:120 | 14.8 | 9.8 | 87.2 |
| LPS373 | DMPC | 1:140 | 15.8 | 6.7 | 100 |
| LPS374 | DMPC | 1:160 | 17.1 | 16.5 | 100 |
| LPS375 | DMPC | 1:180 | 16.6 | 7.5 | 100 |
| LPS376 | POPG | 1:60 | 10.8 | 21.6 | 75.5 |
| LPS377 | POPG | 1:80 | 11.9 | 9.3 | 90.5 |
| LPS378 | POPG | 1:100 | 12.9 | 11.2 | 81.8 |
| LPS379 | POPG | 1:120 | 15.5 | 12.7 | 88.6 |
| LPS380 | POPG | 1:140 | 17.5 | 18.2 | 89.6 |
| LPS381 | POPG | 1:160 | 20.7 | 19.9 | 86.4 |
| LPS382 | POPG | 1:180 | 23.3 | 21.9 | 79 |
| LPS383 | POPS | 1:60 | 11 | 20.7 | 68.7 |
| LPS384 | POPS | 1:80 | 13.8 | 28.1 | 75.5 |
| LPS385 | POPS | 1:100 | 6.7 | 8.4 | 5.1 |
| LPS386 | POPS | 1:120 | 19.0 | 31.9 | 81.6 |
| LPS387 | POPS | 1:140 | 23.3 | 40.6 | 89.9 |
| LPS388 | POPS | 1:160 | 21.9 | 39.4 | 69.2 |
| LPS389 | POPS | 1:180 | 23.9 | 33.6 | 67.3 |

### Conclusions

Novel truncated variants are capable of forming HDL-sized particles with a variety of lipids.

### Example 9: Comparison of Whole Cell Lipid Efflux Capacity of Lipoprotein Systems

### Objective

The present example is directed towards assessing the different functional properties of exemplary lipoprotein systems of the present invention in H4 APOE knockout astroglioma cells. The RedC12 fatty acid probe has been demonstrated to incorporate into cell membranes and neutral lipid stores. Efflux of RedC12 thus represents a measure of whole cell lipid efflux.

### Methods

H4 cells were purchased ATCC, and an APOE knockout clone was generated using CRISPR by Synthego. Cells were cultured in DMEM with L-glutamine media supplemented with 10% non-heat inactivated FBS (Gibco). Cells were passaged every 3-4 days and kept in 37° C., 5% CO₂ incubator. Cells were treated with PBS or lipoprotein discs in HBSS (Gibco).

Further, RedC12 lipid efflux was measured. APOE knockout H4 cells were seeded at 10 k cells/well in 96-well plate. After 6 hours, the media was replaced with 5 μM Red C12 (Thermo) containing media and cells were incubated at 37° C., 5% CO₂ for 18-20 hours. After overnight incubation, cells were washed 3 times with 1×HBSS and treated with 1.2-150 μg/ml lipoprotein discs in HBSS for 2 hours. Following treatment, media was collected into a new 96-well plate and cells were fixed in 4% formaldehyde for 15-20 mins. An equal amount of absolute ethanol was added to HBSS media to obtain optimum Red C12 fluorescence in media. Red C12 fluorescence in media and fixed cells was measured by plate reader at Ex/Em—535/585 nm. Fixed cells were stained with 2 μg/ml Hoechst for 45 min to examine any differences in cell seeding and to monitor any toxicity of treatment. The percentage Red C12 efflux was calculated from media and fixed cell Red C12 fluorescence. The percentage Red C12 efflux of PBS treatment was set to 0 and 150 μg/ml control disc treatment set to 100 for normalization. Dose response curves were fit by non-linear regression in Prism (GraphPad). The EC50 values for RedC12 lipid efflux are summarized in Table 53.

### Results

TABLE 53
of RedC12 lipid efflux EC50 values

| Lipoprotein | EC50 (μg/mL) | SD | n |
|---|---|---|---|
| LPS325 | 3.059 | 0.297 | 2 |
| LPS224 | 3.589 | 0.005657 | 2 |
| LPS252 | 4.309 | 0.01061 | 2 |
| LPS273 | 4.468 | 0.03748 | 2 |
| LPS274 | 5.965 | 0.886 | 2 |
| LPS243 | 6.819 | 1.148 | 2 |
| LPS5 | 7.125 | 1.986 | 2 |
| LPS215 | 7.126 | 2.592 | 2 |
| LPS231 | 12.01 | 0.9405 | 2 |
| LPS286 | 12.94 | 0.3394 | 2 |
| LPS307 | 13.37 | 0.2687 | 2 |
| LPS260 | 14.02 | 0.8061 | 2 |

### Conclusions

The further series of lipoprotein systems have similar RedC12 lipid efflux as PP2 particles. Lipoproteins formulated with DMPC exhibited the greatest potency for RedC12 efflux, whereas lipoproteins formulated with POPG exhibited the least potency. These results suggest that novel variant lipoproteins promote whole cell lipid efflux.

### Example 10: Substrate Analyses of Exemplary Lipoprotein Systems

### Objective

The present example is directed towards analyzing the interaction between the exemplary lipoprotein systems of Example 1 with a series of proteins and receptors. Exemplary proteins and receptors include LCAT, CETP, cell surface lipid transporters, cell surface lipoprotein receptors, and heparin. Further examples are provided for lipid efflux assays and microglial cytokine assays.

### Methods

The goal of this assay is to determine if the novel exemplary lipoprotein systems are substrates for lecithin-cholesterol acyltransferase (LCAT) and if the exemplary lipoprotein systems can be converted from discoidal to spherical shapes. In some instances, suspension ExpiCHO cells are transiently transfected following standard manufacturer's protocols (ThermoFisher) to express and secret human LCAT. The activity units of LCAT in secreted media are quantified with an enzyme-coupled assay (Sigma-Aldrich) and standardized across experiments. In some instances, freshly isolated serum, for example rabbit serum, is used as a source of LCAT. The exemplary lipoprotein systems containing phospholipids and unesterified cholesterol are prepared and incubated with LCAT using experimentally-determined parameters, and conversion of unesterified cholesterol to cholesteryl esters is determined with an cholesteryl esterase/cholesterol oxidase-coupled fluorometric assay (Sigma-Aldrich). The transformation of lipoprotein systems from discoidal to spherical shapes are further characterized by native PAGE, EM, and SEC-MALS.

The goal of this assay is to determine if the exemplary lipoprotein systems are substrates for cholesteryl ester transfer protein (CETP). In some instances, suspension ExpiCHO cells are transiently transfected following standard manufacturer's protocols (ThermoFisher) to express and secret human CETP. The activity units of CETP in secreted media are quantified with an enzyme-coupled assay (Sigma-Aldrich) and standardized across experiments. This assay measures the CETP-mediated transfer of a fluorophore-labeled cholesteryl ester from a donor particle in which its fluorescence is quenched to an acceptor particle with a proportional increase in fluorescence intensity. In some embodiments, the donor particle may be, for example, lipoprotein systems or lipid particles. In some embodiments, the donor particle may be, for example, lipoprotein systems and lipid particles. In some embodiments, the donor particle may be, for example, lipoprotein systems. In some embodiments, the donor particle may be, for example, lipid particles. In some embodiments, the donor particle may be, for example, cholesterol particles. In some embodiments, the acceptor particle may be, for example, lipoprotein systems or lipid particles. In some embodiments, the acceptor particle may be, for example, lipoprotein systems and lipid particles. In some embodiments, the acceptor particle may be, for example, lipoprotein systems. In some embodiments, the acceptor particle may be, for example, lipid particles. In some embodiments, the acceptor particle may be, for example, cholesterol particles.

The same assay used to standardize CETP activity is then modified to measure the transfer of fluorophore-labeled cholesteryl ester to, for example, various exemplary lipoprotein systems as the acceptor particles. In other instances, the exemplary lipoprotein systems containing fluorophore-labeled cholesteryl esters are prepared, and the CETP-mediated transfer of fluorescence to standard donor particles is determined. In some embodiments, the standard donor particles may be, for example, lipoprotein systems or lipid particles. In some embodiments, the standard donor particles may be, for example, lipoprotein systems. In some embodiments, the standard donor particles may be, for example, lipid particles. In some embodiments, the standard donor particles may be, for example, cholesterol particles.

### Receptor-Mediated Uptake Assays

The goal of these assays is to characterize the relative affinities of the exemplary lipoprotein systems for various cell surface receptors. In some instances, HEK293 cells are transiently transfected following standard manufacturer's protocols (ThermoFisher) to overexpress APOE receptors, including human low-density lipoprotein receptor (LDLR), low density lipoprotein receptor-related protein 1 (LRP1), and very low-density lipoprotein receptor (VLDLR). In some instances, binding of the exemplary lipoprotein systems is detected by cell fixation and immunostaining. In some instances, binding of the exemplary lipoprotein systems is detected by fluorescence imaging of a pre-labeled lipoprotein. In some instances, the polypeptides are fluorophore-labeled in denaturing buffer with 1-10 fold molar excess of a dye-conjugated succinimidyl ester (AnaSpec), then purified by SEC and assembled to form the exemplary lipoprotein systems. Dose-dependent uptake of the fluorophore-labeled exemplary lipoprotein systems is measured for each overexpression line, for example, by flow cytometry, and the relative affinity of the novel exemplary lipoprotein systems is determined by competitive binding assays. In some instances, Dil-LDL (ThermoFisher) is used as a positive control. To distinguish between fluorescence bound at the cell surface versus internalized within the cell, samples are mixed with Trypan Blue (ThermoFisher) prior for analysis by flow cytometry to quench extracellular fluorescence. In addition, cells may be treated with recombinant heparinase (Sigma-Aldrich) to digest cell-surface heparan sulfate moieties prior to fluorescence uptake assays. In other instances, uptake of the fluorophore-labeled exemplary lipoprotein systems is measured by microscopy and in other cell lines, including adherent cell lines such as HepG2. Cell lines stably expressing Cas9 may be used to silence expression of endogenously expressed receptors to validate receptor binding affinities.

### Heparin Binding Assay

The goal of this assay is to determine the relative binding affinities of the exemplary lipoprotein systems for heparin sulfate. In some instances, the exemplary lipoprotein systems are loaded onto a HiTrap heparin HP column (GE) in low-salt buffer (20 mM Tris HCl, 20 mM NaCl, pH 7.5) and are eluted with a linear gradient of high salt buffer (20 mM Tris HCl, 1 M NaCl, pH 7.5). The exemplary lipoprotein systems with a higher affinity for heparin had a longer retention time on the column. In other instances, the dose-dependent binding of the exemplary lipoprotein systems to heparin-coated microwell plates is measured by washing the unbound exemplary lipoprotein systems and quantifying the total bound exemplary lipoprotein systems per well using a BCA assay.

### Lipid Efflux Assays

The goal of this assay is to measure the capacity of the exemplary lipoprotein systems to stimulate efflux of cholesterol and peroxidized lipids from cultured cells. The method can be generally applied to any adherent cell line, including H4 astroglioma cells (ATCC) and many other immortalized and primary cell types grown in phenol-red free media. In some instances, cells such as H4 astrogliomas are pretreated with excess lipids, oxidized lipoproteins, or lipid debris to induce lipid droplet formation. After the pretreatment period, the phenol red-free media is refreshed, and the exemplary lipoprotein systems are applied. Transfer of cholesterol and cholesteryl esters to the exogenous exemplary lipoprotein systems in the conditioned media is quantified with the cholesteryl esterase/cholesterol oxidase-coupled fluorometric assay (Sigma-Aldrich). Cell lines stably expressing Cas9 may be used to silence expression of endogenously expressed transporters such as ABCA1 to determine the contribution of these transporters to cholesterol efflux. In other instances, cells such as primary murine neurons are pretreated with chemical reagents to induce oxidative stress as well as a lipid dye such as the BODIPY 581/591 C11 ratiometric fluorescent indicator of lipid oxidation (ThermoFisher). Transfer of oxidized lipid species to the exemplary lipoprotein systems is measured by concentrating the conditioned media with 100 kDa molecular weight cutoff centrifugal filters (Amicon or Sartorius) and measuring the ratio of oxidized and reduced probe fluorescence intensities. In other instances, the lipid species effluxed to the exogenous exemplary lipoprotein systems are analyzed by lipidomics.

### Microglial Cytokine Assay

The goal of this assay is to measure the efficacy of the exemplary lipoprotein systems to reduce levels of inflammatory cytokines secreted by stimulated microglia, including immortalized microglia cells (IMGs, Sigma-Aldrich) and primary microglia. In some instances, microglia are stimulated with excess lipids, oxidized lipoproteins, or other forms of lipid debris including myelin debris. In other instances, apoptotic or ferroptotic cellular debris is applied to induce cytokine release. Proinflammatory stimulants such as lipopolysaccharide are used as a positive control. The exemplary lipoprotein systems are applied at experimentally-determined time points, and the secretion of cytokines, for example TNF-α or IL-6, is measured by ELISA (Peprotech).

The exemplary lipoprotein systems are labeled with the Bolton Hunter reagent (N-succinimidyl-3,4-hydroxy-5-[1251]iodophenyl propionate) in 4° C. sodium borate buffer, pH 8.5, for 30 minutes then dialyzed into saline. The exemplary lipoprotein systems are administered intravenously, intrathecally, and intranasally, and the biodistribution, including brain uptake and/or retention, and decay-corrected half-life is determined by tissue dissection and scintillation counting. In some embodiments, label-free lipoprotein systems are administered intravenously, intrathecally, and/or intranasally, and the biodistribution, including brain uptake and/or retention, is detected by enzyme-linked immunosorbent assay, western blot, or immunohistochemistry. In some embodiments, label-free lipoprotein systems are administered intravenously, intrathecally, intranasally, or by any combination thereof. In some embodiments, label-free lipoprotein systems are administered intravenously. In some embodiments, label-free lipoprotein systems are administered intrathecally. In some embodiments, label-free lipoprotein systems are administered intranasally. In some embodiments, label-free lipoprotein systems are administered by a combination of intravenous, intrathecal, and intranasal administration. In some embodiments, the biodistribution of the label-free lipoprotein systems administered is detected by enzyme-linked immunosorbent assay, western blot, immunohistochemistry, or any combination thereof. In some embodiments, the biodistribution of the label-free lipoprotein systems administered is detected by enzyme-linked immunosorbent assay. In some embodiments, the biodistribution of the label-free lipoprotein systems administered is detected by western blot. In some embodiments, the biodistribution of the label-free lipoprotein systems administered is detected by immunohistochemistry. In some embodiments, the biodistribution of the label-free lipoprotein systems administered is detected by a combination of enzyme-linked immunosorbent assay, western blot, and immunohistochemistry. In some embodiments, the biodistribution of lipoprotein systems includes brain uptake, brain retention, or brain uptake and retention. In some embodiments, the biodistribution of lipoprotein systems includes brain uptake. In some embodiments, the biodistribution of lipoprotein systems includes brain retention. In some embodiments, the biodistribution of lipoprotein systems includes brain uptake and retention. In some embodiments, the biodistribution of lipoprotein systems is brain uptake, brain retention, or brain uptake and retention. In some embodiments, the biodistribution of lipoprotein systems is brain uptake. In some embodiments, the biodistribution of lipoprotein systems is brain retention. In some embodiments, the biodistribution of lipoprotein systems is brain uptake and retention. In some instances, label-free lipoprotein systems are detected by mass spectrometry.

### Equivalents and Scope

Those skilled in the art will recognize or be able to ascertain using no more than routine experimentation many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

It should be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated, the term “about” meaning within an acceptable error range for the particular value should be assumed.

Reference in the specification to “embodiments,” “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

1. A lipoprotein system comprising:
(a) an apolipoprotein E (APOE); and
(b) a lipid;
wherein the lipid is operably associated with the apolipoprotein E.

2. The lipoprotein system of claim 1, wherein the APOE comprises an amino acid sequence that is at least 85% identical to any one of SEQ ID NOs: 267-385.

3. The lipoprotein system of claim 1 or claim 2, wherein the APOE comprises an amino acid sequence that is at least 95% identical to any one of SEQ ID NOs: 267-385.

4. The lipoprotein system of any one of claims 1-3, wherein the APOE comprises an amino acid sequence that is any one of SEQ ID NOs: 267-385.

5. The lipoprotein system of any one of claims 1-4, wherein the APOE comprises one or more mutations relative to the amino acid sequence relative to SEQ ID NO: 1 or SEQ ID NO: 268, wherein the one or more mutations are selected from the list of: A6T (SEQ ID NO: 388), A7V (SEQ ID NO: 389), L9P (SEQ ID NO: 390), V101 (SEQ ID NO: 391), T11A (SEQ ID NO: 392), T11S (SEQ ID NO: 393), F12Y (SEQ ID NO: 394), F12L (SEQ ID NO: 395), L13R (SEQ ID NO: 396), A18P (SEQ ID NO: 397), A18T (SEQ ID NO: 398), E21K (SEQ ID NO: 399), A23V (SEQ ID NO: 400), V24L (SEQ ID NO: 401), T26_E29dup (SEQ ID NO: 402), P28L (SEQ ID NO: 403), E31K (SEQ ID NO: 404), R33C (SEQ ID NO: 405), R33H (SEQ ID NO: 406), Q35R (SEQ ID NO: 407), E37K (SEQ ID NO: 408), Q39R (SEQ ID NO: 409), G41S (SEQ ID NO: 410), R43del (SEQ ID NO: 411), R43C (SEQ ID NO: 412), R43H (SEQ ID NO: 413), L46P (SEQ ID NO: 415), L48V (SEQ ID NO: 416), R50C (SEQ ID NO: 417), R50S (SEQ ID NO: 418), R50G (SEQ ID NO: 419), R50P (SEQ ID NO: 420), R50H (SEQ ID NO: 421), R50L (SEQ ID NO: 422), R56H (SEQ ID NO: 423), T60A (SEQ ID NO: 424), Q64H (SEQ ID NO: 425), E68del (SEQ ID NO: 426), E68V (SEQ ID NO: 428), E68D (SEQ ID NO: 429), L69Q (SEQ ID NO: 430), L69P (SEQ ID NO: 431), L70F (SEQ ID NO: 432), Q73R (SEQ ID NO: 433), V74A (SEQ ID NO: 434), Q76R (SEQ ID NO: 435), E77K (SEQ ID NO: 436), A80E (SEQ ID NO: 437), M82R (SEQ ID NO: 438), D83E (SEQ ID NO: 439), E84K (SEQ ID NO: 440), M86V (SEQ ID NO: 441), E88Q (SEQ ID NO: 442), L89S (SEQ ID NO: 443), K90E (SEQ ID NO: 444), K90N (SEQ ID NO: 445), Y92C (SEQ ID NO: 446), K93fs (SEQ ID NO: 447), K93T (SEQ ID NO: 448), K93R (SEQ ID NO: 449), K93N (SEQ ID NO: 450), S94* (SEQ ID NO: 451), Q99K (SEQ ID NO: 452), Q99R (SEQ ID NO: 453), P102R (SEQ ID NO: 454), V103L (SEQ ID NO: 455), V103L (SEQ ID NO: 456), A104T (SEQ ID NO: 457), E106Q (SEQ ID NO: 458), R108_A118dup (SEQ ID NO: 459), R108Q (SEQ ID NO: 460), R110fs (SEQ ID NO: 461), R110Q (SEQ ID NO: 461), S112Y (SEQ ID NO: 462), K113E (SEQ ID NO: 463), E114K (SEQ ID NO: 464), L115R (SEQ ID NO: 465), A117V (SEQ ID NO: 466), Q119P (SEQ ID NO: 467), A120S (SEQ ID NO: 468), A120D (SEQ ID NO: 469), R121W (SEQ ID NO: 470), G123R (SEQ ID NO: 471), D128N (SEQ ID NO: 472), V129M (SEQ ID NO: 473), G131C (SEQ ID NO: 474), R132C (SEQ ID NO: 475), R132L (SEQ ID NO: 476), Q135R (SEQ ID NO: 477), Y136C (SEQ ID NO: 478), R137C (SEQ ID NO: 479), R137G (SEQ ID NO: 480), R137H (SEQ ID NO: 481), G138C (SEQ ID NO: 482), V140L (SEQ ID NO: 483), V140M (SEQ ID NO: 484), L1441 (SEQ ID NO: 485), G145R (SEQ ID NO: 486), G145S (SEQ ID NO: 487), G145D (SEQ ID NO: 488), G145A (SEQ ID NO: 489), Q146* (SEQ ID NO: 490), L151M (SEQ ID NO: 491), R152W (SEQ ID NO: 492), R152Q (SEQ ID NO: 493), V153M (SEQ ID NO: 494), R154S (SEQ ID NO: 330), R154fs (SEQ ID NO: 495), R154C (SEQ ID NO: 496), R154L (SEQ ID NO: 497), S157F (SEQ ID NO: 498), L159P (SEQ ID NO: 499), R160G (SEQ ID NO: 500), 163C (SEQ ID NO: 501), R163H (SEQ ID NO: 502), K164Q (SEQ ID NO: 503), L167del (SEQ ID NO: 504), R168H (SEQ ID NO: 505), D169Y (SEQ ID NO: 506), D172N (SEQ ID NO: 507), Q174K (SEQ ID NO: 508), V179A (SEQ ID NO: 509), Y180H (SEQ ID NO: 510), Q181E (SEQ ID NO: 511), Q181H (SEQ ID NO: 512), A184S (SEQ ID NO: 513), G187A (SEQ ID NO: 514), G187V (SEQ ID NO: 515), G191_R198dup (SEQ ID NO: 516), E189K (SEQ ID NO: 517), R190C (SEQ ID NO: 518), G191S (SEQ ID NO: 519), S193R (SEQ ID NO: 520), R196C (SEQ ID NO: 521), R198C (SEQ ID NO: 522), G200R (SEQ ID NO: 523), R207S (SEQ ID NO: 524), R207C (SEQ ID NO: 525), R207H (SEQ ID NO: 526), R207P (SEQ ID NO: 527), V208L (SEQ ID NO: 528), V208M (SEQ ID NO: 529), V213G (SEQ ID NO: 530), G214S (SEQ ID NO: 531), G218R (SEQ ID NO: 532), G218S (SEQ ID NO: 533), Q219E (SEQ ID NO: 534), P220L (SEQ ID NO: 535), L221 P (SEQ ID NO: 536), Q222* (SEQ ID NO: 537), Q222K (SEQ ID NO: 538), Q222R (SEQ ID NO: 539), R224P (SEQ ID NO: 540), R224L (SEQ ID NO: 541), A225T (SEQ ID NO: 542), A227S (SEQ ID NO: 543), W228* (SEQ ID NO: 544), G229R (SEQ ID NO: 545), G229V (SEQ ID NO: 546), E230K (SEQ ID NO: 547), R231Q (SEQ ID NO: 548), A234T (SEQ ID NO: 549), A234V (SEQ ID NO: 550), R235W (SEQ ID NO: 551), R235Q (SEQ ID NO: 552), E238del (SEQ ID NO: 553), E238D (SEQ ID NO: 554), M2391 (SEQ ID NO: 555), G240R (SEQ ID NO: 556), S241R (SEQ ID NO: 557), T243fs (SEQ ID NO: 558), R242W (SEQ ID NO: 559), R242G (SEQ ID NO: 560), R242P (SEQ ID NO: 561), T243N (SEQ ID NO: 562), R246S (SEQ ID NO: 563), R246C (SEQ ID NO: 564), R246P (SEQ ID NO: 565), D248E (SEQ ID NO: 566), E249K (SEQ ID NO: 567), E249Q (SEQ ID NO: 568), E256V (SEQ ID NO: 569), R258H (SEQ ID NO: 570), A259P (SEQ ID NO: 571), E262K (SEQ ID NO: 572), E263K (SEQ ID NO: 573), Q267R (SEQ ID NO: 574), V254E (SEQ ID NO: 331), R269fs (SEQ ID NO: 575), R269C (SEQ ID NO: 576), R269G (SEQ ID NO: 577), E273G (SEQ ID NO: 578), A274G (SEQ ID NO: 579), Q276* (SEQ ID NO: 580), A277V (SEQ ID NO: 581), R278C (SEQ ID NO: 582), L279fs (SEQ ID NO: 583), W282C (SEQ ID NO: 584), E284G (SEQ ID NO: 585), E284D (SEQ ID NO: 586), L286M (SEQ ID NO: 587), V287M (SEQ ID NO: 588), D289A (SEQ ID NO: 589), R292C (SEQ ID NO: 590), R292H (SEQ ID NO: 591), W294C (SEQ ID NO: 592), A295T (SEQ ID NO: 593), G296R (SEQ ID NO: 594), G296W (SEQ ID NO: 595), L297P (SEQ ID NO: 596), E299Q (SEQ ID NO: 597), K300R (SEQ ID NO: 598), K300N (SEQ ID NO: 599), V301L (SEQ ID NO: 600), A303S (SEQ ID NO: 601), V305M (SEQ ID NO: 602), T3071 (SEQ ID NO: 603), A309T (SEQ ID NO: 604), P311A (SEQ ID NO: 605), S314R (SEQ ID NO: 606), *318ext (SEQ ID NO: 607), W38* (SEQ ID NO: 608), E98fs (SEQ ID NO: 609), A117T (SEQ ID NO: 610), L122M (SEQ ID NO: 611), Q141R (SEQ ID NO: 612), R160C (SEQ ID NO: 613), R163P (SEQ ID NO: 614), K164E (SEQ ID NO: 615), R165P (SEQ ID NO: 616), A170P (SEQ ID NO: 617), G183A (SEQ ID NO: 618), and combinations thereof.

6. The lipoprotein system of claim 5, wherein the one or more mutations comprise R154S (SEQ ID NO.: 330).

7. The lipoprotein system of claim 5, wherein the one or more mutations comprise V254E (SEQ ID NO.: 331).

8. The lipoprotein system of any one of claims 1-4, wherein the APOE comprises any one of SEQ ID NOs: 275-277.

9. The lipoprotein system of any one of claims 1-8, wherein the APOE comprises one or more sequence regions selected from a signal sequence, an N-terminus region, a structured sequence region, a hinge region, a linker region, and a C-terminus region.

10. The lipoprotein system of claim 9, wherein the APOE comprises more than one structured sequence region.

11. The lipoprotein system of claim 10, wherein each structured sequence region is selected from an alpha helix sequence region, a beta confirmation sequence region, and a beta turn sequence region.

12. The lipoprotein system of any one of claims 1-11, wherein the APOE comprises one or more chemical modifications.

13. The lipoprotein system of claim 12, wherein the one or more chemical modifications comprise one or more post-translational modifications (PTM).

14. The lipoprotein system of claim 13, wherein the one or more post translational modifications are selected from the list of: oxidation, carbamylation, oxi-carbamylation, acetylation, phosphorylation, ubiqutination, biotinylation, carboxylation, deamidation, deamination, deacetylation, dihydroxylation, dephosphorylation, formylation, gamma-carboxyglutamation, glutathionylation, glycation, hydroxylation, methylation, nitration, sumoylation, N- or O-transglutamination, glycosylation and farnesylation.

15. The lipoprotein system of any one of claims 1-14, wherein the APOE comprises an oxidatively coupled dimer, the dimer comprising two APOE monomers.

16. The lipoprotein system of claim 15, wherein the APOE monomer comprises an amino acid sequence at least 85% identical to any one of SEQ ID NOs: 267-385.

17. The lipoprotein system of claim 15 or claim 16, wherein the APOE monomer comprises an amino acid sequence at least 95% identical to any one of SEQ ID NOs: 267-385.

18. The lipoprotein system of any one of claims 15-17, wherein the APOE monomer is any one of SEQ ID NOs: 267-385.

19. The lipoprotein system of any one of claims 15-18, wherein the monomer is any one of SEQ ID NOs: 268, 271, 275-277, or 294, optionally wherein the monomer is SEQ ID NO: 277.

20. The lipoprotein system of any one of claims 1-19, wherein the lipid comprises the formula (I): 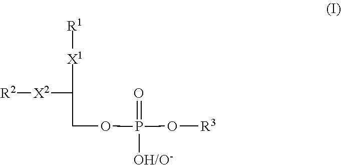
wherein:
X¹ and X² are independently N, O, or absent;
R¹ and R² are independently optionally substituted acyl, optionally substituted alkyl, optionally substituted alkenyl, or absent; and
R³ is selected from:

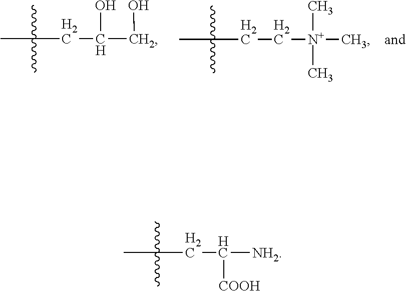

21. The lipoprotein system of any one of claims 1-20, wherein the lipid is a glycerophospholipid.

22. The lipoprotein system of any one of claims 1-21, wherein the lipid is a lysolipid.

23. The lipoprotein system of any one of claims 1-22, wherein the lipid is a phosphatidylglycerol.

24. The lipoprotein system of claim 23, wherein the phosphatidylglycerol is 1-palmitoyl-2-oleoyl-sn-glycero-3-(phospho-rac-(1-glycerol)) (POPG), 1,2-dimyristolyl-sn-glycero-3-phosphoglycerol (DMPG), or 1,2-dioleoyl-sn-glycero-3-phosphoglycerol (DOPG).

25. The lipoprotein system of any one of claims 1-22, wherein the lipid is a phosphatidylcholine.

26. The lipoprotein system of claim 25, wherein the phosphatidylcholine is 1-palmitoyl-2-oleoyl-sn-glycero-3-phosphocholine (POPC), 1,2-dimyristoyl-sn-glycero-3-phosphocholine (DMPC), 1,2-dipalmitoyl-sn-glycero-3-phosphocholine (DPPC), 1,2-dioleoyl-sn-gycero-3-phosphocholine (DOPC), or N-oleoyl-D-erythro-sphingosylphosphorylcholine (18:1 SM).

27. The lipoprotein system of any one of claims 1-22, wherein the lipid is a phosphatidylserine.

28. The lipoprotein system of claim 27, wherein the phosphatidylserine is 1,2-dimyristoyl-sn-glycero-3-phospho-L-serine (DMPS), 1-palmitoyl-2-oleoyl-sn-glycero-3-phospho-L-serine (POPS), or 1,2-dioleoyl-sn-glycero-3-phospho-L-serine (DOPS).

29. The lipoprotein system of any one of claims 1-28, wherein the lipid comprises at least 2 unique lipids.

30. The lipoprotein system of claim 29, wherein the at least two unique lipids comprises a first lipid and a second lipid.

31. The lipoprotein system of claim 30, wherein the molar ratio of the first lipid to the second lipid is from about 1:100 of the first lipid to the second lipid, to about 100:1 of the first lipid to the second lipid.

32. The lipoprotein system of claim 30 or claim 31, wherein the molar ratio of the first lipid to the second lipid is about 1:1 of the first lipid to the second lipid.

33. The lipoprotein system of any one of claims 30-32, wherein the first lipid is POPC and the second lipid is POPS.

34. The lipoprotein system of any one of claims 1-33, wherein the APOE and the lipid are in a molar ratio of from about 1:10000 APOE to lipid, to about 1:1 APOE to lipid.

35. The lipoprotein system of any one of claims 1-34, wherein the APOE and the lipid are in a molar ratio of from about 1:500 APOE to lipid, to about 1:30 APOE to lipid.

36. The lipoprotein system of any one of claims 1-35, wherein the APOE and the lipid are in a molar ratio of from about 1:200 APOE to lipid, to about 1:50 APOE to lipid.

37. The lipoprotein system of any one of claims 1-36, wherein the APOE and the lipid are in a molar ratio of about 1:100 APOE to lipid.

38. The lipoprotein system of any one of claims 1-37, wherein the system has a discoidal, a spherical, a cylindrical, a lamellar, or an ellipsoidal form.

39. The lipoprotein system of any one of claims 1-38, wherein the system has a discoidal form.

40. The lipoprotein system of any one of claims 1-39, wherein the system has a diameter of between 1 nm and 100 nm.

41. The lipoprotein system of any one of claims 1-40, wherein the system has a percent polydispersity of at most 25%.

42. The lipoprotein system of any one of claims 1-41, wherein the system has a percent polydispersity of at most 20%.

43. The lipoprotein system of any one of claims 1-42, wherein the system has a percent polydispersity of at most 15%.

44. The lipoprotein system of any one of claims 1-43, wherein the system has a percent polydispersity of at most 10%.

45. The lipoprotein system of any one of claims 1-44, wherein the system has a percent polydispersity of at most 5%.

46. The lipoprotein system of any one of claims 1-45, further comprising a payload.

47. The lipoprotein system of claim 46, wherein the payload is selected from the group consisting of: peptide payload, a protein payload, a nucleic acid payload, a lipid payload, a lipid derivative payload, a carbohydrate payload, a glycolipid payload, a metabolite payload, a metabolite derivative payload, and a small molecule payload.

48. The lipoprotein system of claim 46 or claim 47, wherein the payload is a small molecule payload.

49. The lipoprotein system of either claim 47 or claim 48, wherein the small molecule is selected from: antioxidant, an antineoplastic agent, an analgesic, an anesthetic, an antibacterial, an anticonvulsant, an antidementia agent, an antidepressant, an antiemetic, an antifungal, an antigout agent, an anti-inflammatory agent, an antimigraine agent, an antimyasthenic agent, an antimycobacterial agent, an antiparasitic, an antiparkinson agent, an antipsychotic, an antispasticity agent, an antiviral, an anxiolytic, a bipolar agent, a blood glucose regulator, a blood product, a blood modifier, a blood volume expander, a chemotherapeutic agent, a cardiovascular agent, a central nervous system agent, a dental oral agent, a dermatological agent, an electrolyte, an enzyme replacement, an enzyme modifier, a gastrointestinal agent, a genitourinary agent, an immunological agent, an inflammatory bowel disease agent, a metabolic bone disease agent, an ophthalmic agent, an otic agent, a respiratory tract agent, a sedative, a hypnotic, and a skeletal muscle relaxant.

50. The lipoprotein system of claim 46 or claim 47, wherein the payload is a nucleic acid.

51. The lipoprotein system of claim 47 or claim 48, wherein the nucleic acid is of RNA, DNA or cDNA.

52. The lipoprotein system of claim 51, wherein the RNA is mRNA, siRNA, microRNA, interference RNA, replicon mRNA, RNA-analogue, guide RNA, or short hairpin RNA (shRNA).

53. The lipoprotein system of any one of claims 1-51, further comprising a lipoprotein system modifier.

54. The lipoprotein system of claim 53, wherein the lipoprotein system modifier is a targeting agent, a regulatory agent, a solubilizing agent, a stabilizing agent, or a detection agent.

55. A method of treating or preventing a disease, or disorder in a subject, the method comprising administering the lipoprotein system of any one of claims 1-54.

56. The method of claim 55, wherein the disease, injury, or disorder is a neurological disease, an autoimmune disease, a cardiovascular disease, a vascular disease, a respiratory disease, a metabolic disease, a gastrointestinal disease, a genetic syndrome, a cancer, or a multisystem disease.

57. The method of claim 55 or claim 56, wherein the disease, injury, or disorder is a neurological disease.

58. The method of any one of claims 55-57, wherein the disease, injury, or disorder is selected from one or more of: Degenerative Cervical Myelopathy, Niemann-Pick disease, amyotrophic lateral sclerosis (ALS), multiple sclerosis, Parkinson's disease, Alzheimer's disease, Huntington's disease, Smith-Lemli Opitz syndrome (SLOS), multiple system atrophy, prion disease, impaired cognitive function, or dementia.

59. The method of claim 57 or 58, wherein the neurological disease is Niemann-Pick disease.

60. The method of claim 57 or 58, wherein the neurological disease is Alzheimer's disease.

61. The method of claim 57, wherein the neurological disease is severe acute respiratory syndrome coronavirus 2 (SARS CoV-2) infection, or a neurological disease resulting from a SARS CoV-2 infection.


SEQUENCE LISTING (881 sequences)

SEQ ID NO: 1 - protein, 317 aa, Homo sapiens [sequence omitted]

SEQ ID NO: 2 - protein, 317 aa, Homo sapiens [sequence omitted]

SEQ ID NO: 3 - protein, 317 aa, Homo sapiens [sequence omitted]

SEQ ID NO: 4 - protein, 216 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSL

SEQ ID NO: 5 - protein, 269 aa, Homo sapiens
MSSGASRKSWDPGNPWPPDWPITGRKMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRT

SEQ ID NO: 6 - protein, 219 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQ

SEQ ID NO: 7 - protein, 343 aa, Homo sapiens
MSSGASRKSWDPGNPWPPDWPITGRKMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 8 - protein, 215 aa, Homo sapiens
MFKQLSCISLPSSCDYSSRSLLQPPSPKMVMLLLLLSALAGLFGAAEGQAFHLGKCPNPPVQENFDVNKYLGRWYEIEKIPTTFENGRCIQANYSLMENGKIKVLNQELRADGTVNQIEGEATPVNLTEPAKLEVKFSWFMPSAPYWILATDYENYALVYSCTCIIQLFHVDFAWILARNPNLPPETVDSLKNILTSNNIDVKKMTVTDQVNCPK

SEQ ID NO: 9 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 10 - protein, 245 aa, Homo sapiens
MSGGARGFCMLKAPHSARPFFSSRSPTALQDESCGADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 11 - protein, 11 aa, synthetic construct
ALMDETMKELK

SEQ ID NO: 12 - protein, 11 aa, synthetic construct
AYKSELEEQLT

SEQ ID NO: 13 - protein, 11 aa, synthetic construct
PVAEETRARLS

SEQ ID NO: 14 - protein, 11 aa, synthetic construct
KELQAAQARLG

SEQ ID NO: 15 - protein, 11 aa, synthetic construct
ADMEDVCGRLV

SEQ ID NO: 16 - protein, 11 aa, synthetic construct
QYRGEVQAMLG

SEQ ID NO: 17 - protein, 11 aa, synthetic construct
QSTEELRVRLA

SEQ ID NO: 18 - protein, 11 aa, synthetic construct
SHLRKLRKRLL

SEQ ID NO: 19 - protein, 11 aa, synthetic construct
RDADDLQKRLA

SEQ ID NO: 20 - protein, 11 aa, synthetic construct
VYQAGAREGAE

SEQ ID NO: 21 - protein, 11 aa, synthetic construct
RGLSAIRERLG

SEQ ID NO: 22 - protein, 11 aa, synthetic construct
DRLDEVKEQVA

SEQ ID NO: 23 - protein, 11 aa, synthetic construct
AKLEEQAQQIR

SEQ ID NO: 24 - protein, 11 aa, synthetic construct
LQAEAFQARLK

SEQ ID NO: 25 - protein, 11 aa, synthetic construct
PLVEDMQRQWA

SEQ ID NO: 26 - protein, 11 aa, synthetic construct
GLVEKVQAAVG

SEQ ID NO: 27 - protein, 11 aa, synthetic construct
QSTEELRVSLA

SEQ ID NO: 28 - protein, 11 aa, synthetic construct
PLAEDMQRQWA

SEQ ID NO: 29 - protein, 11 aa, synthetic construct
GQVEKVQAAEG

SEQ ID NO: 30 - protein, 11 aa, synthetic construct
GQVEKVQAAVG

SEQ ID NO: 31 - protein, 11 aa, synthetic construct
ADMEDVRGRLV

SEQ ID NO: 32 - protein, 11 aa, synthetic construct
RDADDLQKCLA

SEQ ID NO: 33 - protein, 11 aa, synthetic construct
QSTEELRVXLA

SEQ ID NO: 34 - protein, 11 aa, synthetic construct
RDADDLQKXLA

SEQ ID NO: 35 - protein, 11 aa, synthetic construct
DRLDEVKEQVX

SEQ ID NO: 36 - protein, 11 aa, synthetic construct
LQAEAXQARLK

SEQ ID NO: 37 - protein, 11 aa, synthetic construct
GXVEKVQAAXG

SEQ ID NO: 38 - protein, 7 aa, synthetic construct
PLQERAQ

SEQ ID NO: 39 - protein, 9 aa, synthetic construct
AWGERLRAR

SEQ ID NO: 40 - protein, 9 aa, synthetic construct
MEEMGSRTR

SEQ ID NO: 41 - protein, 11 aa, synthetic construct
LKLLDNWDSVT

SEQ ID NO: 42 - protein, 11 aa, synthetic construct
STFSKLREQLG

SEQ ID NO: 43 - protein, 11 aa, synthetic construct
PVTQEFWDNLE

SEQ ID NO: 44 - protein, 11 aa, synthetic construct
KETEGLRQEMS

SEQ ID NO: 45 - protein, 11 aa, synthetic construct
KDLEEVKAKVQ

SEQ ID NO: 46 - protein, 11 aa, synthetic construct
PYLDDFQKKWQ

SEQ ID NO: 47 - protein, 11 aa, synthetic construct
EEMELYRQKVE

SEQ ID NO: 48 - protein, 11 aa, synthetic construct
PLRAELQEGAR

SEQ ID NO: 49 - protein, 11 aa, synthetic construct
QKLHELQEKLS

SEQ ID NO: 50 - protein, 11 aa, synthetic construct
PLGEEMRDRAR

SEQ ID NO: 51 - protein, 11 aa, synthetic construct
AHVDALRTHLA

SEQ ID NO: 52 - protein, 11 aa, synthetic construct
PYSDELRQRLA

SEQ ID NO: 53 - protein, 11 aa, synthetic construct
ARLEALKENGG

SEQ ID NO: 54 - protein, 11 aa, synthetic construct
ARLAEYHAKAT

SEQ ID NO: 55 - protein, 11 aa, synthetic construct
EHLSTLSEKAK

SEQ ID NO: 56 - protein, 11 aa, synthetic construct
PALEDLRQGLL

SEQ ID NO: 57 - protein, 11 aa, synthetic construct
PVLESFKVSFL

SEQ ID NO: 58 - protein, 11 aa, synthetic construct
SALEEYTKKLN

SEQ ID NO: 59 - protein, 11 aa, synthetic construct
ALFQDKLGEVN

SEQ ID NO: 60 - protein, 11 aa, synthetic construct
TYAGDLQKKLV

SEQ ID NO: 61 - protein, 11 aa, synthetic construct
PFATELHERLA

SEQ ID NO: 62 - protein, 11 aa, synthetic construct
KDSEKLKEEIG

SEQ ID NO: 63 - protein, 11 aa, synthetic construct
KELEELRARLL

SEQ ID NO: 64 - protein, 11 aa, synthetic construct
PHANEVSQKIG

SEQ ID NO: 65 - protein, 11 aa, synthetic construct
DNLRELQQRLE

SEQ ID NO: 66 - protein, 11 aa, synthetic construct
PYADQLRTQVN

SEQ ID NO: 67 - protein, 11 aa, synthetic construct
TQAEQLRRQLT

SEQ ID NO: 68 - protein, 11 aa, synthetic construct
PYAQRMERVLR

SEQ ID NO: 69 - protein, 11 aa, synthetic construct
ENADSLQASLR

SEQ ID NO: 70 - protein, 11 aa, synthetic construct
PHADELKAKID

SEQ ID NO: 71 - protein, 11 aa, synthetic construct
QNVEELKGRLT

SEQ ID NO: 72 - protein, 11 aa, synthetic construct
PYADEFKVKID

SEQ ID NO: 73 - protein, 11 aa, synthetic construct
QTVEELRRSLA

SEQ ID NO: 74 - protein, 11 aa, synthetic construct
PYAQDTQEKLN

SEQ ID NO: 75 - protein, 11 aa, synthetic construct
HQLEGLTFQMK

SEQ ID NO: 76 - protein, 11 aa, synthetic construct
KNAEELKARIS

SEQ ID NO: 77 - protein, 11 aa, synthetic construct
ASAEELRQRLA

SEQ ID NO: 78 - protein, 11 aa, synthetic construct
PLAEDVRGNLR

SEQ ID NO: 79 - protein, 11 aa, synthetic construct
QQVEEFRRRVE

SEQ ID NO: 80 - protein, 11 aa, synthetic construct
PYGENFNKALV

SEQ ID NO: 81 - protein, 11 aa, synthetic construct
QQMEQLRQKLG

SEQ ID NO: 82 - protein, 11 aa, synthetic construct
PHAGDVEGHLS

SEQ ID NO: 83 - protein, 11 aa, synthetic construct
FLEKDLRDKVN

SEQ ID NO: 84 - protein, 11 aa, synthetic construct
PLXEELXXRLX

SEQ ID NO: 85 - protein, 11 aa, synthetic construct
PLXEEXRXXLX

SEQ ID NO: 86 - protein, 11 aa, synthetic construct
XXLEELREKLX

SEQ ID NO: 87 - protein, 11 aa, synthetic construct
XXXEEXQXRLX

SEQ ID NO: 88 - protein, 11 aa, synthetic construct
LQAEAAQARLK

SEQ ID NO: 89 - protein, 11 aa, synthetic construct
GLVEKVQAAEG

SEQ ID NO: 90 - protein, 11 aa, synthetic construct
DRLDEVKEQEA

SEQ ID NO: 91 - protein, 7 aa, synthetic construct
ELRVRLA

SEQ ID NO: 92 - protein, 5 aa, synthetic construct
CGRLV

SEQ ID NO: 93 - protein, 6 aa, synthetic construct
PLVEDM

SEQ ID NO: 94 - protein, 4 aa, synthetic construct
VRLA

SEQ ID NO: 95 - protein, 8 aa, synthetic construct
ARLKENGG

SEQ ID NO: 96 - protein, 11 aa, synthetic construct
PYSDELRQCLA

SEQ ID NO: 97 - protein, 11 aa, synthetic construct
RDADDLQCRLA

SEQ ID NO: 98 - protein, 61 aa, synthetic construct
KVEQAVETEPEPELRQQTEWQSGQRWELALGRFWDYLRWVQTLSEQVQEELLSSQVTQELR

SEQ ID NO: 99 - protein, 43 aa, synthetic construct
DEPPQSPWDRVKDLATVYVDVLKDSGRDYVSQFEGSALGKQLN

SEQ ID NO: 100 - protein, 39 aa, synthetic construct
GQRWELALGRFWDYLRWVQTLSEQVQEELLSSQVTQELR

SEQ ID NO: 101 - protein, 17 aa, synthetic construct
EQVQEELLSSQVTQELR

SEQ ID NO: 102 - protein, 7 aa, synthetic construct
QVTQELR

SEQ ID NO: 103 - protein, 11 aa, synthetic construct
TSAAPVPSDNH

SEQ ID NO: 104 - protein, 4 aa, synthetic construct
GGGG

SEQ ID NO: 105 - protein, 6 aa, synthetic construct
GGGGGG

SEQ ID NO: 106 - protein, 6 aa, synthetic construct
GGSGGS

SEQ ID NO: 107 - protein, 9 aa, synthetic construct
GGSGGSGGS

SEQ ID NO: 108 - protein, 12 aa, synthetic construct
GGSGGSGGSGGS

SEQ ID NO: 109 - protein, 8 aa, synthetic construct
GGGGGGGG

SEQ ID NO: 110 - protein, 4 aa, synthetic construct
GGGS

SEQ ID NO: 111 - protein, 5 aa, synthetic construct
GGGGS

SEQ ID NO: 112 - protein, 10 aa, synthetic construct
GGGGSGGGGS

SEQ ID NO: 113 - protein, 15 aa, synthetic construct
GGGGSGGGGSGGGGS

SEQ ID NO: 114 - protein, 20 aa, synthetic construct
GGGGSGGGGSGGGGSGGGGS

SEQ ID NO: 115 - protein, 36 aa, synthetic construct
GGSGGGSEGGGSEGGGSEGGGSEGGGSEGGGSGGGS

SEQ ID NO: 116 - protein, 5 aa, synthetic construct
SSSSG

SEQ ID NO: 117 - protein, 10 aa, synthetic construct
SSSSGSSSSG

SEQ ID NO: 118 - protein, 13 aa, synthetic construct
SGSSSSGSSSSGS

SEQ ID NO: 119 - protein, 15 aa, synthetic construct
SSSSGSSSSGSSSSG

SEQ ID NO: 120 - protein, 19 aa, synthetic construct
SVTVSSSGSSSSGSSSSGS

SEQ ID NO: 121 - protein, 20 aa, synthetic construct
SSSSGSSSSGSSSSGSSSSG

SEQ ID NO: 122 - protein, 25 aa, synthetic construct
SSSSGSSSSGSSSSGSSSSGSSSSG

SEQ ID NO: 123 - protein, 6 aa, synthetic construct
EFSTEF

SEQ ID NO: 124 - protein, 5 aa, synthetic construct
GGSGG

SEQ ID NO: 125 - protein, 4 aa, synthetic construct
GGSG

SEQ ID NO: 126 - protein, 6 aa, synthetic construct
GGSGGG

SEQ ID NO: 127 - protein, 26 aa, synthetic construct
SGGGSGGGGSGGGGSGGGGSGGGSLQ

SEQ ID NO: 128 - protein, 5 aa, synthetic construct
SGGGS

SEQ ID NO: 129 - protein, 20 aa, synthetic construct
SGGGSGGGGSGGGGSGGGGS

SEQ ID NO: 130 - protein, 9 aa, synthetic construct
GGSGGGSGG

SEQ ID NO: 131 - protein, 5 aa, synthetic construct
GGGGG

SEQ ID NO: 132 - protein, 25 aa, synthetic construct
GGGGSGGGGSGGGGSGGGGSGGGGS

SEQ ID NO: 133 - protein, 30 aa, synthetic construct
GGGGSGGGGSGGGGSGGGGSGGGGSGGGGS

SEQ ID NO: 134 - protein, 11 aa, synthetic construct
SSSSGSSSSGG

SEQ ID NO: 135 - protein, 30 aa, synthetic construct
SSSSGSSSSGSSSSGSSSSGSSSSGSSSSG

SEQ ID NO: 136 - protein, 34 aa, synthetic construct
SGGGSGGGGSGGGGSGGGGSGGGGSGGGGSGGGS

SEQ ID NO: 137 - protein, 36 aa, synthetic construct
GGSAGGSGSGSSGGSSGASGTGTAGGTGSGSGTGSG

SEQ ID NO: 138 - protein, 12 aa, synthetic construct
GKSSGSGSESKS

SEQ ID NO: 139 - protein, 15 aa, synthetic construct
GGSTSGSGKSSEGKG

SEQ ID NO: 140 - protein, 19 aa, synthetic construct
GSTSGSGKSSSEGSGSTKG

SEQ ID NO: 141 - protein, 13 aa, synthetic construct
VDYPYDVPDYALD

SEQ ID NO: 142 - protein, 14 aa, synthetic construct
EGKSSGSGSESKEF

SEQ ID NO: 143 - protein, 35 aa, synthetic construct
SGGGSGGGGSGGGGSGGGGSGGGSLQYPYDVPDYA

SEQ ID NO: 144 - protein, 8 aa, synthetic construct
DYKDDDDK

SEQ ID NO: 145 - protein, 34 aa, synthetic construct
SGGGSGGGGSGGGGSGGGGSYPYDVPDYASGGGS

SEQ ID NO: 146 - protein, 22 aa, synthetic construct
GSGATNFSLLKQAGDVEENPGP

SEQ ID NO: 147 - protein, 8 aa, synthetic construct
DVEXNPGP

SEQ ID NO: 148 - protein, 8 aa, synthetic construct
DIEXNPGP

SEQ ID NO: 149 - protein, 22 aa, synthetic construct
SGGGGSGGGGSGGGGSGGGSLQ

SEQ ID NO: 150 - protein, 13 aa, synthetic construct
QLIGMLQGLMRDL

SEQ ID NO: 151 - protein, 4 aa, synthetic construct
ASFE

SEQ ID NO: 152 - protein, 6 aa, synthetic construct
ATKASK

SEQ ID NO: 153 - protein, 7 aa, synthetic construct
ATKGATK

SEQ ID NO: 154 - protein, 7 aa, synthetic construct
IAKAFKN

SEQ ID NO: 155 - protein, 14 aa, synthetic construct
VRGSPAINVAVHVF

SEQ ID NO: 156 - protein, 18 aa, synthetic construct
GSTSGSGKPGSGEGSTKG

SEQ ID NO: 157 - protein, 18 aa, synthetic construct
KESGSVSSEQLAQFRSLD

SEQ ID NO: 158 - protein, 4 aa, synthetic construct
GFLG

SEQ ID NO: 159 - protein, 6 aa, synthetic construct
PLGLWA

SEQ ID NO: 160 - protein, 6 aa, synthetic construct
RVLAEA

SEQ ID NO: 161 - protein, 8 aa, synthetic construct
GGIEGRGS

SEQ ID NO: 162 - protein, 9 aa, synthetic construct
RRRRRRRRR

SEQ ID NO: 163 - protein, 10 aa, synthetic construct
EDVVCCSMSY

SEQ ID NO: 164 - protein, 10 aa, synthetic construct
TRHRQPRGWE

SEQ ID NO: 165 - protein, 10 aa, synthetic construct
AGNRVRRSVG

SEQ ID NO: 166 - protein, 12 aa, synthetic construct
CRRRRRREAEAC

SEQ ID NO: 167 - protein, 17 aa, synthetic construct
VSQTSKLTRAETVFPDV

SEQ ID NO: 168 - protein, 20 aa, synthetic construct
LEAGCKNFFPRSFTSCGSLE

SEQ ID NO: 169 - protein, 12 aa, synthetic construct
GSAGSAAGSGEF

SEQ ID NO: 170 - protein, 14 aa, synthetic construct
EGKSSGSGSESKST

SEQ ID NO: 171 - protein, 20 aa, synthetic construct
NSGAGSGGSSGSDGASGSRD

SEQ ID NO: 172 - protein, 5 aa, synthetic construct
GSLDG

SEQ ID NO: 173 - protein, 4 aa, synthetic construct
SLDG

SEQ ID NO: 174 - protein, 12 aa, synthetic construct
GSGGGSGGGSGS

SEQ ID NO: 175 - protein, 11 aa, synthetic construct
GSGSGSGSGSG

SEQ ID NO: 176 - protein, 16 aa, synthetic construct
GGGGSLVPRGSGGGGS

SEQ ID NO: 177 - protein, 15 aa, synthetic construct
GGGGSLVPRGSGGGG

SEQ ID NO: 178 - protein, 11 aa, synthetic construct
GGSGGHMGSGG

SEQ ID NO: 179 - protein, 11 aa, synthetic construct
GGSGGSGGSGG

SEQ ID NO: 180 - protein, 18 aa, synthetic construct
GGGSEGGGSEGGGSEGGG

SEQ ID NO: 181 - protein, 8 aa, synthetic construct
GGSGGGSG

SEQ ID NO: 182 - protein, 4 aa, synthetic construct
SWFE

SEQ ID NO: 183 - protein, 4 aa, synthetic construct
SRFE

SEQ ID NO: 184 - protein, 44 aa, synthetic construct
PLVEQGRVRAATVGSLAGQPLQERAQAWGERLRARMEEMGSRTR

SEQ ID NO: 185 - protein, 16 aa, synthetic construct
RAATVGSLAGQPLQER

SEQ ID NO: 186 - protein, 27 aa, synthetic construct
RAATVGSLAGQPLQERAQAWGERLRAR

SEQ ID NO: 187 - protein, 19 aa, synthetic construct
PLVEQGRVRAATVGSLAGQ

SEQ ID NO: 188 - protein, 25 aa, synthetic construct
PLQERAQAWGERLRARMEEMGSRTR

SEQ ID NO: 189 - protein, 11 aa, synthetic construct
PLVEQGRVRAA

SEQ ID NO: 190 - protein, 22 aa, synthetic construct
PLVEQGRVRADATVGSLAGQLG

SEQ ID NO: 191 - protein, 46 aa, synthetic construct
PLVEQGRVRADATVGSLAGQLGAQAWGERLRARLGPLMEEMGSRTR

SEQ ID NO: 192 - protein, 44 aa, synthetic construct
PLVEQGRVRAAAVGSLAGQPLQERAQAWGERLRARMEEMGSRTR

SEQ ID NO: 193 - protein, 8 aa, synthetic construct
KQEHFPDN

SEQ ID NO: 194 - protein, 16 aa, synthetic construct
VEPKSPDKTHTCPPCP

SEQ ID NO: 195 - protein, 16 aa, synthetic construct
LDPKSSDKTHTCPPCP

SEQ ID NO: 196 - protein, 39 aa, synthetic construct
IEVMYPPPYLDNEKSNGTIIHVKGKHLCPSPLFPGPSKP

SEQ ID NO: 197 - protein, 12 aa, synthetic construct
GGAVHTRGLDFA

SEQ ID NO: 198 - protein, 45 aa, synthetic construct
TTTPAPRPPTPAPTIASQPLSLRPEACRPAAGGAVHTRGLDFACD

SEQ ID NO: 199 - protein, 48 aa, synthetic construct
AKPTTTPAPRPPTPAPTIASQPLSLRPEACRPAAGGAVHTRGLDFACD

SEQ ID NO: 200 - protein, 79 aa, synthetic construct
TTTPAPRPPTPAPTIASQPLSLRPEACRPAAGGAVHTRGLDFACDEPKSPDKTHTCPPCPAPPVAGPSVFLFPPKPKDT

SEQ ID NO: 201 - protein, 6 aa, synthetic construct
LDFACD

SEQ ID NO: 202 - protein, 51 aa, synthetic construct
PAKPTTTPAPRPPTPAPTIASQPLSLRPEACRPAAGGAVHTRGLDFACDIY

SEQ ID NO: 203 - protein, 44 aa, synthetic construct
TTPAPRPPTPAPTIASQPLSLRPEACRPAAGGAVHTRGLDFACD

SEQ ID NO: 204 - protein, 47 aa, synthetic construct
TTTPAPRPPTPAPTIASQPLSLRPEACRPAAGGAVHTRGLDFACDIY

SEQ ID NO: 205 - protein, 11 aa, synthetic construct
LDKTHTCPPCP

SEQ ID NO: 206 - protein, 37 aa, synthetic construct
FSIPQANHSHSGDYHCTGNIGYTLYSSKPVTITVQAP

SEQ ID NO: 207 - protein, 16 aa, synthetic construct
GLAVSTISSFFPPGYQ

SEQ ID NO: 208 - protein, 5 aa, synthetic construct
DKTHT

SEQ ID NO: 209 - protein, 4 aa, synthetic construct
CPPC

SEQ ID NO: 210 - protein, 15 aa, synthetic construct
CPEPKSCDTPPPCPR

SEQ ID NO: 211 - protein, 12 aa, synthetic construct
ELKTPLGDTTHT

SEQ ID NO: 212 - protein, 10 aa, synthetic construct
KSCDKTHTCP

SEQ ID NO: 213 - protein, 7 aa, synthetic construct
KCCVDCP

SEQ ID NO: 214 - protein, 7 aa, synthetic construct
KYGPPCP

SEQ ID NO: 215 - protein, 246 aa, synthetic construct
YVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 216 - protein, 45 aa, synthetic construct
KPTTTPAPRPPTPAPTIASQPLSLRPEACRPAAGGAVHTRGLDFA

SEQ ID NO: 217 - protein, 16 aa, synthetic construct
LEPKSCDKTHTCPPCP

SEQ ID NO: 218 - protein, 43 aa, synthetic construct
KPTTTPAPRPPTPAPTIASQPLSLRPEACRPAAGGAVHTRGLD

SEQ ID NO: 219 - protein, 16 aa, synthetic construct
ELKTPLGDTHTCPRCP

SEQ ID NO: 220 - protein, 15 aa, synthetic construct
EPKSCDTPPPCPRCP

SEQ ID NO: 221 - protein, 12 aa, synthetic construct
ESKYGPPCPSCP

SEQ ID NO: 222 - protein, 12 aa, synthetic construct
ERKCCVECPPCP

SEQ ID NO: 223 - protein, 282 aa, synthetic construct
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 224 - protein, 281 aa, synthetic construct
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 225 - protein, 283 aa, synthetic construct
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMVVLEDQREVNTSGFAPARPPPQPGSTTFWAWSVLRVPAPPSPQPATYTCVVSHEDSRTLLNASRSLEVSYVTDH

SEQ ID NO: 226 - protein, 58 aa, synthetic construct
ESPKAQASSVPTAQPQAEGSLAKATTAPATTRNTGRGGEEKKKEKEKEEQEERETKTP

SEQ ID NO: 227 - protein, 234 aa, synthetic construct
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 228 - protein, 15 aa, synthetic construct
EPKSCDKTHTCPPCP

SEQ ID NO: 229 - protein, 20 aa, synthetic construct
AEPKSPDKTHTCPPCPKDPK

SEQ ID NO: 230 - protein, 233 aa, synthetic construct
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEVKCKVSNKALPAPIEKTISKAKGQPREPQVYTLPPSRDELTKNQVSLTCLVKGFYPSDIAVEWESNGQPENNYKTTPPVLDSDGSFFLVSKLTVDKSRWQQGNVFSCSVMHEALHNHYTQKSLSLSPGKKD

SEQ ID NO: 231 - protein, 13 aa, synthetic construct
SVFLFPPKPKDTL

SEQ ID NO: 232 - protein, 235 aa, synthetic construct
DPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKK

SEQ ID NO: 233 - protein, 231 aa, synthetic construct
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 234 - protein, 235 aa, synthetic construct
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 235 - protein, 223 aa, synthetic construct
VECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - protein, 17 aa, synthetic construct
ELKTPLGDTTHTCPRCP

SEQ ID NO: 237 - protein, 61 aa, synthetic construct
ELKTPLGDTHTCPRCPEPKSCDTPPPCPRCPEPKSCDTPPPCPRCPEPKSCDTPPPCPRCP

SEQ ID NO: 238 - protein, 12 aa, synthetic construct
SPNMVPHAHHAQ

SEQ ID NO: 239 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 240 - protein, 129 aa, synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 241 - protein, 229 aa, synthetic construct
ESKYGPPCPSCPAPEFEGGPSVFLFPPKPKDTLMISRTPEVTCVVVDVSQEDPEVQFNWYVDGVEVHQAKTKPREEQFNSTYRVVSVLTVLHQDWLNGKEYKCKVSNKGLPSSIEKTISKAKGQPREPQVYTLPPSQEEMTKNQVSLTCLVKGFVPSDIAVEWESNGQPENNYKTTPPVLDSDGSFFLYSRLTVDKSRWQEGNVFSCSVMHEALHNHYTQKSLSLSLGK

SEQ ID NO: 242 - protein, 229 aa, synthetic construct
ESKYGPPCPPCPAPEFEGGPSVFLFPPKPKDTLMISRTPEVTCVVVDVSQEDPEVQFNWYVDGVEVHQAKTKPREEQFNSTYRVVSVLTVLHQDWLNGKEYKCKVSNKGLPSSIEKTISKAKGQPREPQVYTLPPSQEEMTKNQVSLTCLVKGFVPSDIAVEWESNGQPENNYKTTPPVLDSDGSFFLYSRLTVDKSRWQEGNVFSCSVMHEALHNHYTQKSLSLSLGK

SEQ ID NO: 243 - protein, 12 aa, synthetic construct
ESKYGPPCPPCP

SEQ ID NO: 244 - protein, 9 aa, synthetic construct
YGPPCPPCP

SEQ ID NO: 245 - protein, 10 aa, synthetic construct
KYGPPCPPCP

SEQ ID NO: 246 - protein, 13 aa, synthetic construct
EVVKYGPPCPPCP

SEQ ID NO: 247 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDLSRWQEGNVFSCSVMHEALHNHYTQKSLSLSLGK

SEQ ID NO: 248 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 249 - protein, 119 aa, synthetic construct
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 250 - protein, 231 aa, synthetic construct
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTIPPVLDSDGSFFLYSKLTVDKSRWQQGNVFSCSVMHEALHNHYTQKSLSLSPGK

SEQ ID NO: 251 - protein, 231 aa, synthetic construct
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPLEKTISKAKGQPREPQVYTLPPSRDELTKNQVSLTCLVKGFYPSDIAVEWESNGQPENNYKTIPPVLDSDGSFFLYSKLTVDKSRWQQGNVFSCSVMHEALHNHYTQKSLSLSPGK

SEQ ID NO: 252 - protein, 18 aa, synthetic construct
MKVLWAALLVTFLAGCQA

SEQ ID NO: 253 - protein, 18 aa, synthetic construct
MKAAVLTLAVLFLTGSQA

SEQ ID NO: 254 - protein, 18 aa, synthetic construct
MKLLAATVLLLTICSLEG

SEQ ID NO: 255 - protein, 20 aa, synthetic construct
MFLKAVVLTLALVAVAGARA

SEQ ID NO: 256 - protein, 27 aa, synthetic construct
MDPPRPALLALLALPALLLLLLAGARA

SEQ ID NO: 257 - protein, 22 aa, synthetic construct
MGTRLLPALFLVLLVLGFEVQG

SEQ ID NO: 258 - protein, 20 aa, synthetic construct
MQPRVLLVVALLALLASARA

SEQ ID NO: 259 - protein, 26 aa, synthetic construct
MRLFLSLPVLVVVLSIVLEGPAPAQG

SEQ ID NO: 260 - protein, 20 aa, synthetic construct
MVMLLLLLSALAGLFGAAEG

SEQ ID NO: 261 - protein, 22 aa, synthetic construct
MFHQIWAALLYFYGIILNSIYQ

SEQ ID NO: 262 - protein, 19 aa, synthetic construct
MEHKEVVLLLLLFLKSAAP

SEQ ID NO: 263 - protein, 21 aa, synthetic construct
METDTLLLWVLLLWVPGSTGD

SEQ ID NO: 264 - protein, 20 aa, synthetic construct
METDTLLLWVLLLWVPGSTG

SEQ ID NO: 265 - protein, 36 aa, synthetic construct
MKVLWAALLVTFLAGCQAMKVLWAALLVTFLAGCQA

SEQ ID NO: 266 - protein, 24 aa, synthetic construct
MKAAVLTLAVLFLTGSQARHFWQQ

SEQ ID NO: 267 - protein, 299 aa, Homo sapiens
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKCLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 268 - protein, 299 aa, Homo sapiens; identical to SEQ ID NO: 267

SEQ ID NO: 269 - protein, 299 aa, Homo sapiens
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVRGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 270 - protein, 299 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAAQARLKSRFEPLAEDMQRQWAGQVEKVQAAEGTSAAPVPSDNH

SEQ ID NO: 271 - protein, 299 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 272 - protein, 227 aa, synthetic construct
ALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVG

SEQ ID NO: 273 - protein, 238 aa, synthetic construct
ALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 274 - protein, 288 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVG

SEQ ID NO: 275 - protein, 292 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAFQARLKPLVEDMQRQWAGLVEKVQAAVGTSAAPVPSDNH

SEQ ID NO: 276 - protein, 255 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGDRLDEVKEQVAEVRAKLEEQAQQIRLQAEAFQARLKSWFEPLVEDMQRQWAGLVEKVQAAVGTSAAPVPSDNH

SEQ ID NO: 277 - protein, 248 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGDRLDEVKEQVAAKLEEQAQQIRLQAEAFQARLKPLVEDMQRQWAGLVEKVQAAVGTSAAPVPSDNH

SEQ ID NO: 278 - protein, 292 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAFQARLKPLVEDMQRQWAGLVEKVQAAVGTSAAPVPSDNH

SEQ ID NO: 279 - protein, 292 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVRGRLVQYRGEVQAMLGQSTEELRVRLASHLRKLRKRLLRDADDLQKCLAVYQAGAREGAERGLSAIRERLGPLVEQGRVRAATVGSLAGQPLQERAQAWGERLRARMEEMGSRTRDRLDEVKEQVAAKLEEQAQQIRLQAEAFQARLKPLVEDMQRQWAGLVEKVQAAVGTSAAPVPSDNH

SEQ ID NO: 280 - protein, 281 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAFQARLKPLVEDMQRQWAGLVEKVQAAVG

SEQ ID NO: 281 - protein, 292 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAAQARLKPLAEDMQRQWAGQVEKVQAAEGTSAAPVPSDNH

SEQ ID NO: 282 - protein, 281 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAAQARLKPLAEDMQRQWAGQVEKVQAAEG

SEQ ID NO: 283 - protein, 292 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAAQARLKPLAEDMQRQWAGQVEKVQAAEGTSAAPVPSDNH

SEQ ID NO: 284 - protein, 292 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVRGRLVQYRGEVQAMLGQSTEELRVRLASHLRKLRKRLLRDADDLQKCLAVYQAGAREGAERGLSAIRERLGPLVEQGRVRAATVGSLAGQPLQERAQAWGERLRARMEEMGSRTRDRLDEVKEQVAAKLEEQAQQIRLQAEAAQARLKPLAEDMQRQWAGQVEKVQAAEGTSAAPVPSDNH

SEQ ID NO: 285 - protein, 292 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAAQARLKPLVEDMQRQWAGLVEKVQAAVGTSAAPVPSDNH

SEQ ID NO: 286 - protein, 292 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAFQARLKPLAEDMQRQWAGLVEKVQAAVGTSAAPVPSDNH

SEQ ID NO: 287 - protein, 292 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAFQARLKPLVEDMQRQWAGQVEKVQAAVGTSAAPVPSDNH

SEQ ID NO: 288 - protein, 292 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAFQARLKPLVEDMQRQWAGLVEKVQAAEGTSAAPVPSDNH

SEQ ID NO: 289 - protein, 198 aa, synthetic construct
STFSKLREQLGALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGDRLDEVKEQVAAKLEEQAQQIRLQAEAFQARLKPLVEDMQRQWAGLVEKVQAAVGTSAAPVPSDNH

SEQ ID NO: 290 - protein, 209 aa, synthetic construct
STFSKLREQLGPVTQEFWDNLEALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGDRLDEVKEQVAAKLEEQAQQIRLQAEAFQARLKPLVEDMQRQWAGLVEKVQAAVGTSAAPVPSDNH

SEQ ID NO: 291 - protein, 220 aa, synthetic construct
STFSKLREQLGPVTQEFWDNLEKETEGLRQEMSALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGDRLDEVKEQVAAKLEEQAQQIRLQAEAFQARLKPLVEDMQRQWAGLVEKVQAAVGTSAAPVPSDNH

SEQ ID NO: 292 - protein, 292 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVRGRLVQYRGEVQAMLGQSTEELRVSLASHLRKLRKRLLRDADDLQKCLAVYQAGAREGAERGLSAIRERLGPLVEQGRVRAATVGSLAGQPLQERAQAWGERLRARMEEMGSRTRDRLDEVKEQVAAKLEEQAQQIRLQAEAFQARLKPLVEDMQRQWAGLVEKVQAAVGTSAAPVPSDNH

SEQ ID NO: 293 - protein, 209 aa, synthetic construct
STFSKLREQLGPVTQEFWDNLEKETEGLRQEMSALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGDRLDEVKEQVAAKLEEQAQQIRLQAEAFQARLKPLVEDMQRQWAGLVEKVQAAVG

SEQ ID NO: 294 - protein, 299 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQEAEVRAKLEEQAQQIRLQAEAFQARLKSWFEPLVEDMQRQWAGLVEKVQAAVGTSAAPVPSDNH

SEQ ID NO: 295 - protein, 189 aa, synthetic construct
STFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 296 - protein, 243 aa, synthetic construct
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 297 - protein, 292 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAAQARLKPLVEDMQRQWAGLVEKVQAAVGTSAAPVPSDNH

SEQ ID NO: 298 - protein, 292 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQEAAKLEEQAQQIRLQAEAAQARLKPLVEDMQRQWAGLVEKVQAAVGTSAAPVPSDNH

SEQ ID NO: 299 - protein, 292 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQEAAKLEEQAQQIRLQAEAAQARLKPLVEDMQRQWAGLVEKVQAAVGTSAAPVPSDNH

SEQ ID NO: 300 - protein, 248 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGDRLDEVKEQVAAKLEEQAQQIRLQAEAAQARLKPLVEDMQRQWAGLVEKVQAAVGTSAAPVPSDNH

SEQ ID NO: 301 - protein, 248 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGDRLDEVKEQEAAKLEEQAQQIRLQAEAAQARLKPLVEDMQRQWAGLVEKVQAAVGTSAAPVPSDNH

SEQ ID NO: 302 - protein, 248 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGDRLDEVKEQEAAKLEEQAQQIRLQAEAAQARLKPLVEDMQRQWAGLVEKVQAAVGTSAAPVPSDNH

SEQ ID NO: 303 - protein, 259 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAADRLDEVKEQVAAKLEEQAQQIRLQAEAFQARLKPLVEDMQRQWAGLVEKVQAAVGTSAAPVPSDNH

SEQ ID NO: 304 - protein, 270 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRADATVGSLAGQLGDRLDEVKEQVAAKLEEQAQQIRLQAEAFQARLKPLVEDMQRQWAGLVEKVQAAVGTSAAPVPSDNH

SEQ ID NO: 305 - protein, 294 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRADATVGSLAGQLGAQAWGERLRARLGPLMEEMGSRTRDRLDEVKEQVAAKLEEQAQQIRLQAEAFQARLKPLVEDMQRQWAGLVEKVQAAVGTSAAPVPSDNH

SEQ ID NO: 306 - protein, 243 aa, synthetic construct
DEPPQSPWDRVKDLATVYVDVLKDSGRDYVSQFEGSALGKQLNLKLLDNWDSVTSTFSKLREQLGPVTQEFWDNLEKETEGLRQEMSADMEDVCGRLVQYRGEVQAMLGQSTEELRVRLASHLRKLRKRLLRDADDLQKRLAVYQAGAREGAEAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 307 - protein, 298 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGKDLEEVKAKVQPYLDDFQKKWQEEMELYRQKVEPLRAELQEGARQKLHELQEKLSPLGEEMRDRARGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 308 - protein, 248 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGKDLEEVKAKVQPYLDDFQKKWQEEMELYRQKVEPLRAELQEGARQKLHELQEKLSPLGEEMRDRARRGLSAIRERLGDRLDEVKEQVAAKLEEQAQQIRLQAEAFQARLKPLVEDMQRQWAGLVEKVQAAVGTSAAPVPSDNH

SEQ ID NO: 309 - protein, 189 aa, synthetic construct
STFSKLREQLGPVTQEFWDNLEKETEGLRQEMSKDLEEVKAKVQADMEDVCGRLVQYRGEVQAMLGQSTEELRVRLASHLRKLRKRLLRDADDLQKRLAVYQAGAREGAEPYSDELRQRLAARLEALKENGGARLAEYHAKATEHLSTLSEKAKPALEDLRQGLLPVLESFKVSFLSALEEYTKKLNTQ

SEQ ID NO: 310 - protein, 220 aa, synthetic construct
ALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAFQARLKPLVEDMQRQWAGLVEKVQAAVG

SEQ ID NO: 311 - protein, 176 aa, synthetic construct
ALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGDRLDEVKEQVAAKLEEQAQQIRLQAEAFQARLKPLVEDMQRQWAGLVEKVQAAVG

SEQ ID NO: 312 - protein, 179 aa, synthetic construct
ALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGGGSDRLDEVKEQVAAKLEEQAQQIRLQAEAFQARLKPLVEDMQRQWAGLVEKVQAAVG

SEQ ID NO: 313 - protein, 182 aa, synthetic construct
ALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGGGSGGSDRLDEVKEQVAAKLEEQAQQIRLQAEAFQARLKPLVEDMQRQWAGLVEKVQAAVG

SEQ ID NO: 314 - protein, 185 aa, synthetic construct
ALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGGGSGGSGGSDRLDEVKEQVAAKLEEQAQQIRLQAEAFQARLKPLVEDMQRQWAGLVEKVQAAVG

SEQ ID NO: 315 - protein, 188 aa, synthetic construct
ALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGGGSGGSGGSGGSDRLDEVKEQVAAKLEEQAQQIRLQAEAFQARLKPLVEDMQRQWAGLVEKVQAAVG

SEQ ID NO: 316 - protein, 179 aa, synthetic construct
ALMDETMKELKAYKSELEEQLTPVAEETRARLSKELQAAQARLGADMEDVCGRLVQYRGEVQAMLGQSTEELRVRLASHLRKLRKRLLRDADDLQKCLAVYQAGAREGAERGLSAIRERLGGGSDRLDEVKEQVAAKLEEQAQQIRLQAEAFQARLKPLVEDMQRQWAGLVEKVQAAVG

SEQ ID NO: 317 - protein, 182 aa, synthetic construct
ALMDETMKELKAYKSELEEQLTPVAEETRARLSKELQAAQARLGADMEDVCGRLVQYRGEVQAMLGQSTEELRVRLASHLRKLRKRLLRDADDLQKCLAVYQAGAREGAERGLSAIRERLGGGSGGSDRLDEVKEQVAAKLEEQAQQIRLQAEAFQARLKPLVEDMQRQWAGLVEKVQAAVG

SEQ ID NO: 318 - protein, 185 aa, synthetic construct
ALMDETMKELKAYKSELEEQLTPVAEETRARLSKELQAAQARLGADMEDVCGRLVQYRGEVQAMLGQSTEELRVRLASHLRKLRKRLLRDADDLQKCLAVYQAGAREGAERGLSAIRERLGGGSGGSGGSDRLDEVKEQVAAKLEEQAQQIRLQAEAFQARLKPLVEDMQRQWAGLVEKVQAAVG

SEQ ID NO: 319 - protein, 188 aa, synthetic construct
ALMDETMKELKAYKSELEEQLTPVAEETRARLSKELQAAQARLGADMEDVCGRLVQYRGEVQAMLGQSTEELRVRLASHLRKLRKRLLRDADDLQKCLAVYQAGAREGAERGLSAIRERLGGGSGGSGGSGGSDRLDEVKEQVAAKLEEQAQQIRLQAEAFQARLKPLVEDMQRQWAGLVEKVQAAVG

SEQ ID NO: 320 - protein, 179 aa, synthetic construct
ALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGGGSDRLDEVKEQVAAKLEEQAQQIRLQAEAFQARLKPLVEDMQRQWAGLVEKVQAAVG

SEQ ID NO: 321 - protein, 182 aa, synthetic construct
ALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGGGSGGSDRLDEVKEQVAAKLEEQAQQIRLQAEAFQARLKPLVEDMQRQWAGLVEKVQAAVG

SEQ ID NO: 322 - protein, 185 aa, synthetic construct
ALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGGGSGGSGGSDRLDEVKEQVAAKLEEQAQQIRLQAEAFQARLKPLVEDMQRQWAGLVEKVQAAVG

SEQ ID NO: 323 - protein, 188 aa, synthetic construct
ALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGGGSGGSGGSGGSDRLDEVKEQVAAKLEEQAQQIRLQAEAFQARLKPLVEDMQRQWAGLVEKVQAAVG

SEQ ID NO: 324 - protein, 168 aa, synthetic construct
ALMDETMKELKAYKSELEEQLTPVAEETRARLSADMEDVCGRLVQYRGEVQAMLGQSTEELRVSLASHLRKLRKRLLRDADDLQKRLAVYQAGAREGAERGLSAIRERLGGGSDRLDEVKEQVAAKLEEQAQQIRLQAEAAQARLKPLAEDMQRQWAGQVEKVQAAEG

SEQ ID NO: 325 - protein, 171 aa, synthetic construct
ALMDETMKELKAYKSELEEQLTPVAEETRARLSADMEDVCGRLVQYRGEVQAMLGQSTEELRVSLASHLRKLRKRLLRDADDLQKRLAVYQAGAREGAERGLSAIRERLGGGSGGSDRLDEVKEQVAAKLEEQAQQIRLQAEAAQARLKPLAEDMQRQWAGQVEKVQAAEG

SEQ ID NO: 326 - protein, 174 aa, synthetic construct
ALMDETMKELKAYKSELEEQLTPVAEETRARLSADMEDVCGRLVQYRGEVQAMLGQSTEELRVSLASHLRKLRKRLLRDADDLQKRLAVYQAGAREGAERGLSAIRERLGGGSGGSGGSDRLDEVKEQVAAKLEEQAQQIRLQAEAAQARLKPLAEDMQRQWAGQVEKVQAAEG

SEQ ID NO: 327 - protein, 180 aa, synthetic construct
ALMDETMKELKAYKSELEEQLTPVAEETRARLSADMEDVCGRLVQYRGEVQAMLGQSTEELRVSLASHLRKLRKRLLRDADDLQKRLAVYQAGAREGAERGLSAIRERLGGGSGGSGGSGGSGGSDRLDEVKEQVAAKLEEQAQQIRLQAEAAQARLKPLAEDMQRQWAGQVEKVQAAEG

SEQ ID NO: 328 - protein, 288 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSADMEDVRGRLVQYRGEVQAMLGQSTEELRVRLASHLRKLRKRLLRDADDLQKCLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 329 - protein, 323 aa, synthetic construct
MKAAVLTLAVLFLTGSQARHFWQQKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 330 - protein, 317 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAAQARLKSRFEPLAEDMQRQWAGQVEKVQAAEGTSAAPVPSDNH

SEQ ID NO: 331 - protein, 317 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 332 - protein, 263 aa, synthetic construct
MKVLWAALLVTFLAGCQAMKVLWAALLVTFLAGCQAALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVG

SEQ ID NO: 333 - protein, 263 aa, synthetic construct
MKVLWAALLVTFLAGCQAMKVLWAALLVTFLAGCQAALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAAAVGSLAGQPLQERAQAWGERLRARMEEMGSRTRDRLDEVKEQVAEVRAKLEEQAQQIRLQAEAFQARLKSWFEPLVEDMQRQWAGLVEKVQAAVG

SEQ ID NO: 334 - protein, 335 aa, synthetic construct
MKVLWAALLVTFLAGCQAMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAAAVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 335 - protein, 274 aa, synthetic construct
MKVLWAALLVTFLAGCQAMKVLWAALLVTFLAGCQAALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 336 - protein, 324 aa, synthetic construct
MKVLWAALLVTFLAGCQAMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVG

SEQ ID NO: 337 - protein, 263 aa, synthetic construct
MKVLWAALLVTFLAGCQAMKVLWAALLVTFLAGCQAALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVG

SEQ ID NO: 338 - protein, 263 aa, synthetic construct
MKVLWAALLVTFLAGCQAMKVLWAALLVTFLAGCQAALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAAAVGSLAGQPLQERAQAWGERLRARMEEMGSRTRDRLDEVKEQVAEVRAKLEEQAQQIRLQAEAFQARLKSWFEPLVEDMQRQWAGLVEKVQAAVG

SEQ ID NO: 339 - protein, 317 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAAAVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 340 - protein, 312 aa, synthetic construct
MKVLWAALLVTFLAGCQAEGELEVTDQLPGQSDQPWEQALNRFWDYLRWVQTLSDQVQEELQSSQVTQELTVLMEDTMTEVKAYKKELEEQLGPVAEETRARLAKEVQAAQARLGADMEDLRNRLGQYRNEVNTMLGQSTEELRSRLSTHLRKMRKRLMRDADDLQKRLAVYKAGAQEGAERGVSAIRERLGPLVEQGRQRTANLGAGAAQPLRDRAQALSDRIRGRLEEVGNQARDRLEEVREQMEEVRSKMEEQTQQIRLQAEIFQARIKGWFEPLVEDMQRQWANLMEKIQASVATNSIASTTVPLENQ

SEQ ID NO: 341 - protein, 295 aa, synthetic construct
MKVLWAALLVTFLAGCQAGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 342 - protein, 273 aa, synthetic construct
MKVLWAALLVTFLAGCQAEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 343 - protein, 263 aa, synthetic construct
MKVLWAALLVTFLAGCQAQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 344 - protein, 226 aa, synthetic construct
MKVLWAALLVTFLAGCQARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 345 - protein, 186 aa, synthetic construct
MKVLWAALLVTFLAGCQAELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 346 - protein, 179 aa, synthetic construct
MKVLWAALLVTFLAGCQACGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDM

SEQ ID NO: 347 - protein, 156 aa, synthetic construct
MKVLWAALLVTFLAGCQAVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDM

SEQ ID NO: 348 - protein, 310 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAFQARLKPLVEDMQRQWAGLVEKVQAAVGTSAAPVPSDNH

SEQ ID NO: 349 - protein, 273 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGDRLDEVKEQVAEVRAKLEEQAQQIRLQAEAFQARLKSWFEPLVEDMQRQWAGLVEKVQAAVGTSAAPVPSDNH

SEQ ID NO: 350 - protein, 266 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGDRLDEVKEQVAAKLEEQAQQIRLQAEAFQARLKPLVEDMQRQWAGLVEKVQAAVGTSAAPVPSDNH

SEQ ID NO: 351 - protein, 317 aa, synthetic construct [sequence omitted]

SEQ ID NO: 352 - protein, 207 aa, synthetic construct
MKVLWAALLVTFLAGCQASTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 353 - protein, 218 aa, synthetic construct
MKVLWAALLVTFLAGCQALKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 354 - protein, 204 aa, synthetic construct
MKVLWAALLVTFLAGCQASTFSKLREQLGPVTQEFWDNLEKETEGLRQEMSKDLEEVKAKVQPYLDDFQKKWQEEMELYRQKVEQSTEELRVRLASHLRKLRKRLLRDADDLQKRLAAHVDALRTHLAPYSDELRQRLAARLKENGGARLAEYHAKATEHLSTLSEKAKPALEDLRQGLLPVLESFKVSFLSALEEYTKKLNTQ

SEQ ID NO: 355 - protein, 215 aa, synthetic construct
MKVLWAALLVTFLAGCQALKLLDNWDSVTSTFSKLREQLGPVTQEFWDNLEKETEGLRQEMSKDLEEVKAKVQPYLDDFQKKWQEEMELYRQKVEQSTEELRVRLASHLRKLRKRLLRDADDLQKRLAAHVDALRTHLAPYSDELRQRLAARLKENGGARLAEYHAKATEHLSTLSEKAKPALEDLRQGLLPVLESFKVSFLSALEEYTKKLNTQ

SEQ ID NO: 356 - protein, 319 aa, synthetic construct
METDTLLLWVLLLWVPGSTGKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAAQARLKSRFEPLAEDMQRQWAGQVEKVQAAEGTSAAPVPSDNH

SEQ ID NO: 357 - protein, 310 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAFQARLKPLVEDMQRQWAGLVEKVQAAVGTSAAPVPSDNH

SEQ ID NO: 358 - protein, 309 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVRGRLVQYRGEVQAMLGQSTEELRVRLSHLRKLRKRLLRDADDLQCRLAVYQAGAREGAERGLSAIRERLGPLVEQGRVRAATVGSLAGQPLQERAQAWGERLRARMEEMGSRTRDRLDEVKEQVAAKLEEQAQQIRLQAEAFQARLKPLVEDMQRQWAGLVEKVQAAVGTSAAPVPSDNH

SEQ ID NO: 359 - protein, 299 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAFQARLKPLVEDMQRQWAGLVEKVQAAVG

SEQ ID NO: 360 - protein, 310 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAAQARLKPLAEDMQRQWAGQVEKVQAAEGTSAAPVPSDNH

SEQ ID NO: 361 - protein, 299 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAAQARLKPLAEDMQRQWAGQVEKVQAAEG

SEQ ID NO: 362 - protein, 310 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAAQARLKPLAEDMQRQWAGQVEKVQAAEGTSAAPVPSDNH

SEQ ID NO: 363 - protein, 310 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVRGRLVQYRGEVQAMLGQSTEELRVRLASHLRKLRKRLLRDADDLQKCLAVYQAGAREGAERGLSAIRERLGPLVEQGRVRAATVGSLAGQPLQERAQAWGERLRARMEEMGSRTRDRLDEVKEQVAAKLEEQAQQIRLQAEAAQARLKPLAEDMQRQWAGQVEKVQAAEGTSAAPVPSDNH

SEQ ID NO: 364 - protein, 310 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAAQARLKPLVEDMQRQWAGLVEKVQAAVGTSAAPVPSDNH

SEQ ID NO: 365 - protein, 310 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAFQARLKPLAEDMQRQWAGLVEKVQAAVGTSAAPVPSDNH

SEQ ID NO: 366 - protein, 310 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAFQARLKPLVEDMQRQWAGQVEKVQAAVGTSAAPVPSDNH

SEQ ID NO: 367 - protein, 310 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAFQARLKPLVEDMQRQWAGLVEKVQAAEGTSAAPVPSDNH

SEQ ID NO: 368 - protein, 216 aa, synthetic construct
MKVLWAALLVTFLAGCQASTFSKLREQLGALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGDRLDEVKEQVAAKLEEQAQQIRLQAEAFQARLKPLVEDMQRQWAGLVEKVQAAVGTSAAPVPSDNH

SEQ ID NO: 369 - protein, 227 aa, synthetic construct
MKVLWAALLVTFLAGCQASTFSKLREQLGPVTQEFWDNLEALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGDRLDEVKEQVAAKLEEQAQQIRLQAEAFQARLKPLVEDMQRQWAGLVEKVQAAVGTSAAPVPSDNH

SEQ ID NO: 370 - protein, 238 aa, synthetic construct
MKVLWAALLVTFLAGCQASTFSKLREQLGPVTQEFWDNLEKETEGLRQEMSALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGDRLDEVKEQVAAKLEEQAQQIRLQAEAFQARLKPLVEDMQRQWAGLVEKVQAAVGTSAAPVPSDNH

SEQ ID NO: 371 - protein, 227 aa, synthetic construct
MKVLWAALLVTFLAGCQASTFSKLREQLGPVTQEFWDNLEKETEGLRQEMSALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGDRLDEVKEQVAAKLEEQAQQIRLQAEAFQARLKPLVEDMQRQWAGLVEKVQAAVG

SEQ ID NO: 372 - protein, 227 aa, synthetic construct
MKVLWAALLVTFLAGCQASTFSKLREQLGPVTQEFWDNLEKETEGLRQEMSALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGDRLDEVKEQVAAKLEEQAQQIRLQAEAAQARLKPLAEDMQRQWAGQVEKVQAAEG

SEQ ID NO: 373 - protein, 317 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQEAEVRAKLEEQAQQIRLQAEAFQARLKSWFEPLVEDMQRQWAGLVEKVQAAVGTSAAPVPSDNH

SEQ ID NO: 374 - protein, 267 aa, synthetic construct
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 375 - protein, 310 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQEAAKLEEQAQQIRLQAEAFQARLKPLVEDMQRQWAGLVEKVQAAVGTSAAPVPSDNH

SEQ ID NO: 376 - protein, 310 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQEAAKLEEQAQQIRLQAEAFQARLKPLVEDMQRQWAGLVEKVQAAVGTSAAPVPSDNH

SEQ ID NO: 377 - protein, 310 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAAKLEEQAQQIRLQAEAFQARLKPLVEDMQRQWAGQVEKVQAAVGTSAAPVPSDNH

SEQ ID NO: 378 - protein, 310 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQEAAKLEEQAQQIRLQAEAFQARLKPLVEDMQRQWAGQVEKVQAAVGTSAAPVPSDNH

SEQ ID NO: 379 - protein, 310 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQEAAKLEEQAQQIRLQAEAFQARLKPLVEDMQRQWAGQVEKVQAAVGTSAAPVPSDNH

SEQ ID NO: 380 - protein, 266 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGDRLDEVKEQVAAKLEEQAQQIRLQAEAFQARLKPLVEDMQRQWAGLVEKVQAAVGTSAAPVPSDNH

SEQ ID NO: 381 - protein, 266 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGDRLDEVKEQEAAKLEEQAQQIRLQAEAFQARLKPLVEDMQRQWAGLVEKVQAAVGTSAAPVPSDNH

SEQ ID NO: 382 - protein, 266 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGDRLDEVKEQEAAKLEEQAQQIRLQAEAFQARLKPLVEDMQRQWAGLVEKVQAAVGTSAAPVPSDNH

SEQ ID NO: 383 - protein, 266 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGDRLDEVKEQVAAKLEEQAQQIRLQAEAFQARLKPLVEDMQRQWAGQVEKVQAAVGTSAAPVPSDNH

SEQ ID NO: 384 - protein, 266 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGDRLDEVKEQEAAKLEEQAQQIRLQAEAFQARLKPLVEDMQRQWAGQVEKVQAAVGTSAAPVPSDNH

SEQ ID NO: 385 - protein, 266 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGDRLDEVKEQEAAKLEEQAQQIRLQAEAFQARLKPLVEDMQRQWAGQVEKVQAAVGTSAAPVPSDNH

SEQ ID NO: 386 - protein, 79 aa, Homo sapiens
MKVLWAGLRCWSHSWQDARPRWSKRWRQSRSPSCASRPSGRAASAGNWHWVAFGITCAGCRHCLSRCRRSCSAPRSPRN

SEQ ID NO: 387 - protein, 4 aa, Homo sapiens
MKVL

SEQ ID NO: 388 - protein, 317 aa, Homo sapiens
MKVLWTALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 389 - protein, 317 aa, Homo sapiens
MKVLWAVLLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 390 - protein, 317 aa, Homo sapiens
MKVLWAALPVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 391 - protein, 317 aa, Homo sapiens
MKVLWAALLITFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 392 - protein, 317 aa, Homo sapiens
MKVLWAALLVAFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 393 - protein, 317 aa, Homo sapiens
MKVLWAALLVSFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 394 - protein, 317 aa, Homo sapiens
MKVLWAALLVTYLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 395 - protein, 317 aa, Homo sapiens
MKVLWAALLVTLLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 396 - protein, 317 aa, Homo sapiens
MKVLWAALLVTFRAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 397 - protein, 317 aa, Homo sapiens
MKVLWAALLVTFLAGCQPKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 398 - protein, 317 aa, Homo sapiens [sequence omitted]

SEQ ID NO: 399 - protein, 317 aa, Homo sapiens
MKVLWAALLVTFLAGCQAKVKQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 400 - protein, 317 aa, Homo sapiens
MKVLWAALLVTFLAGCQAKVEQVVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 401 - protein, 317 aa, Homo sapiens
MKVLWAALLVTFLAGCQAKVEQALETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 402 - protein, 321 aa, Homo sapiens
MKVLWAALLVTFLAGCQAKVEQAVETEPETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 403 - protein, 317 aa, Homo sapiens
MKVLWAALLVTFLAGCQAKVEQAVETELEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 404 - protein, 317 aa, Homo sapiens [sequence omitted]

SEQ ID NO: 405 - protein, 317 aa, Homo sapiens
MKVLWAALLVTFLAGCQAKVEQAVETEPEPELCQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 406 - protein, 317 aa, Homo sapiens
MKVLWAALLVTFLAGCQAKVEQAVETEPEPELHQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 407 - protein, 317 aa, Homo sapiens
MKVLWAALLVTFLAGCQAKVEQAVETEPEPELRQRTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 408 - protein, 317 aa, Homo sapiens
MKVLWAALLVTFLAGCQAKVEQAVETEPEPELRQQTKWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 409 - protein, 317 aa, Homo sapiens [sequence omitted]

SEQ ID NO: 410 - protein, 317 aa, Homo sapiens
MKVLWAALLVTFLAGCQAKVEQAVETEPEPELRQQTEWQSSQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 411 - protein, 312 aa, Homo sapiens
MKVLWAALLVTFLAGCQAKVEQAVETEPEPELRQQTEWQSGQLGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 412 - protein, 317 aa, Homo sapiens
MKVLWAALLVTFLAGCQAKVEQAVETEPEPELRQQTEWQSGQCWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 413 - protein, 317 aa, Homo sapiens
MKVLWAALLVTFLAGCQAKVEQAVETEPEPELRQQTEWQSGQHWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 414 - protein, 43 aa, Homo sapiens
MKVLWAALLVTFLAGCQAKVEQAVETEPEPELRQQTEWQSGQR

SEQ ID NO: 415 - protein, 317 aa, Homo sapiens
MKVLWAALLVTFLAGCQAKVEQAVETEPEPELRQQTEWQSGQRWEPALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 416 - protein, 317 aa, Homo sapiens
MKVLWAALLVTFLAGCQAKVEQAVETEPEPELRQQTEWQSGQRWELAVGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 417 - protein, 317 aa, Homo sapiens
MKVLWAALLVTFLAGCQAKVEQAVETEPEPELRQQTEWQSGQRWELALGCFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 418 - protein, 317 aa, Homo sapiens
MKVLWAALLVTFLAGCQAKVEQAVETEPEPELRQQTEWQSGQRWELALGSFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 419 - protein, 317 aa, Homo sapiens
MKVLWAALLVTFLAGCQAKVEQAVETEPEPELRQQTEWQSGQRWELALGGFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 420 - protein, 317 aa, Homo sapiens
MKVLWAALLVTFLAGCQAKVEQAVETEPEPELRQQTEWQSGQRWELALGPFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 421 - protein, 317 aa, Homo sapiens
MKVLWAALLVTFLAGCQAKVEQAVETEPEPELRQQTEWQSGQRWELALGHFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 422 - protein, 317 aa, Homo sapiens
MKVLWAALLVTFLAGCQAKVEQAVETEPEPELRQQTEWQSGQRWELALGLFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 423 - protein, 317 aa, Homo sapiens
MKVLWAALLVTFLAGCQAKVEQAVETEPEPELRQQTEWQSGQRWELALGRFWDYLHWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 424 - protein, 317 aa, Homo sapiens
MKVLWAALLVTFLAGCQAKVEQAVETEPEPELRQQTEWQSGQRWELALGRFWDYLRWVQALSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 425 - protein, 317 aa, Homo sapiens
MKVLWAALLVTFLAGCQAKVEQAVETEPEPELRQQTEWQSGQRWELALGRFWDYLRWVQTLSEHVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 426 - protein, 316 aa, Homo sapiens
MKVLWAALLVTFLAGCQAKVEQAVETEPEPELRQQTEWQSGQRWELALGRFWDYLRWVQTLSEQVQELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 427 - protein, 67 aa, Homo sapiens
MKVLWAALLVTFLAGCQAKVEQAVETEPEPELRQQTEWQSGQRWELALGRFWDYLRWVQTLSEQVQE

SEQ ID NO: 428 - protein, 317 aa, Homo sapiens
MKVLWAALLVTFLAGCQAKVEQAVETEPEPELRQQTEWQSGQRWELALGRFWDYLRWVQTLSEQVQEVLLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 429 - protein, 317 aa, Homo sapiens
MKVLWAALLVTFLAGCQAKVEQAVETEPEPELRQQTEWQSGQRWELALGRFWDYLRWVQTLSEQVQEDLLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 430 - protein, 317 aa, Homo sapiens
MKVLWAALLVTFLAGCQAKVEQAVETEPEPELRQQTEWQSGQRWELALGRFWDYLRWVQTLSEQVQEEQLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 431 - protein, 317 aa, Homo sapiens
MKVLWAALLVTFLAGCQAKVEQAVETEPEPELRQQTEWQSGQRWELALGRFWDYLRWVQTLSEQVQEEPLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 432 - protein, 317 aa, Homo sapiens
MKVLWAALLVTFLAGCQAKVEQAVETEPEPELRQQTEWQSGQRWELALGRFWDYLRWVQTLSEQVQEELFSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 433 - protein, 317 aa, Homo sapiens
MKVLWAALLVTFLAGCQAKVEQAVETEPEPELRQQTEWQSGQRWELALGRFWDYLRWVQTLSEQVQEELLSSRVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 434 - protein, 317 aa, Homo sapiens
MKVLWAALLVTFLAGCQAKVEQAVETEPEPELRQQTEWQSGQRWELALGRFWDYLRWVQTLSEQVQEELLSSQATQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 435 - protein, 317 aa, Homo sapiens
MKVLWAALLVTFLAGCQAKVEQAVETEPEPELRQQTEWQSGQRWELALGRFWDYLRWVQTLSEQVQEELLSSQVTRELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 436 - protein, 317 aa, Homo sapiens
MKVLWAALLVTFLAGCQAKVEQAVETEPEPELRQQTEWQSGQRWELALGRFWDYLRWVQTLSEQVQEELLSSQVTQKLRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 437 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRELMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 438 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALRDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 439 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMEETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 440 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDKTMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 441 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETVKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 442 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKQLKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 443 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKESKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 444 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELEAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 445 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELNAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 446 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKACKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 447 - protein, 162 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYIGTGGTTDPGGGGDAGTAVQGAAGGAGPAGRGHGGRVRPPGAVPRRGAGHARPEHRGAAGAPRLPPAQAA

SEQ ID NO: 448 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYTSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 449 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYRSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 450 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYNSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 451 - protein, 93 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYK

SEQ ID NO: 452 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEKLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 453 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEERLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 454 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTRVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 455 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPLAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 456 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVTEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 457 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEQTRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 458 - protein, 328 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAARARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 459 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETQARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 460 - protein, 163 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRATAVQGAAGGAGPAGRGHGGRVRPPGAVPRRGAGHARPEHRGAAGAPRLPPAQAA

SEQ ID NO: 461 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRAQLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 462 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLYKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 463 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSEELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 464 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKKLQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 465 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKERQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 466 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQVAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 467 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAPARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 468 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQSRLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 469 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQDRLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 470 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQAWLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 471 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLRADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 472 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMENVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 473 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDMCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 474 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCCRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 475 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGCLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 476 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGLLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 477 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVRYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 478 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQCRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 479 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYCGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 480 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYGGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 481 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYHGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 482 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRCEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 483 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGELQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 484 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEMQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 485 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMIGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 486 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLRQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 487 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLSQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 488 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLDQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 489 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLAQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 490 - protein, 145 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLG

SEQ ID NO: 491 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEEMRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 492 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELWVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 493 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELQVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 494 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRMRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 495 - protein, 175 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSGKGWAAEELRVPPRLPPAQAA

SEQ ID NO: 496 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVCLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 497 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVLLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 498 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLAFHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 499 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHPRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 500 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLGKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 501 - protein, 317 aa, Homo sapiens [sequence omitted]

SEQ ID NO: 502 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLHKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 503 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRQRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 504 - protein, 316 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 505 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLHDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 506 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRYADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 507 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADNLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 508 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLKKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 509 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAAYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 510 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVHQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 511 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYEAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 512 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYHAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 513 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGSREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 514 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREAAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 515 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREVAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 516 - protein, 325 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 517 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAKRGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 518 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAECGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 519 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERSLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 520 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLRAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 521 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAICERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 522 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRECLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 523 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLRPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 524 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGSVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 525 - protein, 317 aa, Homo sapiens [sequence omitted]

SEQ ID NO: 526 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGHVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 527 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGPVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 528 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRLRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 529 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRMRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 530 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATGGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 531 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVSSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 532 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLARQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 533 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLASQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 534 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGEPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 535 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQLLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 536 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPPQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 537 - protein, 221 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPL

SEQ ID NO: 538 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLKERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 539 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLRERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 540 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQEPAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 541 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQELAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 542 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERTQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 543 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQSWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 544 - protein, 227 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQA

SEQ ID NO: 545 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWRERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 546 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWVERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 547 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGKRLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 548 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGEQLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 549 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRTRMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 550 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRVRMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 551 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRAWMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 552 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRAQMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 553 - protein, 316 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 554 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEDMGSRTRDRLDEVKEQVAEVRAKLEEQAQQIRLQAEAFQARLKSWFEPLVEDMQRQWAGLVEKVQAAVGTSAAPVPSDNH

SEQ ID NO: 555 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEIGSRTRDRLDEVKEQVAEVRAKLEEQAQQIRLQAEAFQARLKSWFEPLVEDMQRQWAGLVEKVQAAVGTSAAPVPSDNH

SEQ ID NO: 556 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMRSRTRDRLDEVKEQVAEVRAKLEEQAQQIRLQAEAFQARLKSWFEPLVEDMQRQWAGLVEKVQAAVGTSAAPVPSDNH

SEQ ID NO: 557 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGRRTRDRLDEVKEQVAEVRAKLEEQAQQIRLQAEAFQARLKSWFEPLVEDMQRQWAGLVEKVQAAVGTSAAPVPSDNH

SEQ ID NO: 558 - protein, 304 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRRCAPSWRSRPSRYACRPRPSRPASRAGSSPWWKTCSASGPGWWRRCRLPWAPAPPLCPATIT

SEQ ID NO: 559 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSWTRDRLDEVKEQVAEVRAKLEEQAQQIRLQAEAFQARLKSWFEPLVEDMQRQWAGLVEKVQAAVGTSAAPVPSDNH

SEQ ID NO: 560 - protein, 317 aa, Homo sapiens [sequence omitted]

SEQ ID NO: 561 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSPTRDRLDEVKEQVAEVRAKLEEQAQQIRLQAEAFQARLKSWFEPLVEDMQRQWAGLVEKVQAAVGTSAAPVPSDNH

SEQ ID NO: 562 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRNRDRLDEVKEQVAEVRAKLEEQAQQIRLQAEAFQARLKSWFEPLVEDMQRQWAGLVEKVQAAVGTSAAPVPSDNH

SEQ ID NO: 563 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDSLDEVKEQVAEVRAKLEEQAQQIRLQAEAFQARLKSWFEPLVEDMQRQWAGLVEKVQAAVGTSAAPVPSDNH

SEQ ID NO: 564 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDCLDEVKEQVAEVRAKLEEQAQQIRLQAEAFQARLKSWFEPLVEDMQRQWAGLVEKVQAAVGTSAAPVPSDNH

SEQ ID NO: 565 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDPLDEVKEQVAEVRAKLEEQAQQIRLQAEAFQARLKSWFEPLVEDMQRQWAGLVEKVQAAVGTSAAPVPSDNH

SEQ ID NO: 566 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLEEVKEQVAEVRAKLEEQAQQIRLQAEAFQARLKSWFEPLVEDMQRQWAGLVEKVQAAVGTSAAPVPSDNH

SEQ ID NO: 567 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDKVKEQVAEVRAKLEEQAQQIRLQAEAFQARLKSWFEPLVEDMQRQWAGLVEKVQAAVGTSAAPVPSDNH

SEQ ID NO: 568 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDQVKEQVAEVRAKLEEQAQQIRLQAEAFQARLKSWFEPLVEDMQRQWAGLVEKVQAAVGTSAAPVPSDNH

SEQ ID NO: 569 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAVVRAKLEEQAQQIRLQAEAFQARLKSWFEPLVEDMQRQWAGLVEKVQAAVGTSAAPVPSDNH

SEQ ID NO: 570 - protein, 317 aa, Homo sapiens [sequence omitted]

SEQ ID NO: 571 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRPKLEEQAQQIRLQAEAFQARLKSWFEPLVEDMQRQWAGLVEKVQAAVGTSAAPVPSDNH

SEQ ID NO: 572 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLKEQAQQIRLQAEAFQARLKSWFEPLVEDMQRQWAGLVEKVQAAVGTSAAPVPSDNH

SEQ ID NO: 573 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEKQAQQIRLQAEAFQARLKSWFEPLVEDMQRQWAGLVEKVQAAVGTSAAPVPSDNH

SEQ ID NO: 574 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQRIRLQAEAFQARLKSWFEPLVEDMQRQWAGLVEKVQAAVGTSAAPVPSDNH

SEQ ID NO: 575 - protein, 318 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQITPAGRGLPGPPQELVRAPGGRHAAPVGRAGGEGAGCRGHQRRPCAQRQSL

SEQ ID NO: 576 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQICLQAEAFQARLKSWFEPLVEDMQRQWAGLVEKVQAAVGTSAAPVPSDNH

SEQ ID NO: 577 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIGLQAEAFQARLKSWFEPLVEDMQRQWAGLVEKVQAAVGTSAAPVPSDNH

SEQ ID NO: 578 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAGAFQARLKSWFEPLVEDMQRQWAGLVEKVQAAVGTSAAPVPSDNH

SEQ ID NO: 579 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEGFQARLKSWFEPLVEDMQRQWAGLVEKVQAAVGTSAAPVPSDNH

SEQ ID NO: 580 - protein, 275 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAF

SEQ ID NO: 581 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQVRLKSWFEPLVEDMQRQWAGLVEKVQAAVGTSAAPVPSDNH

SEQ ID NO: 582 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQACLKSWFEPLVEDMQRQWAGLVEKVQAAVGTSAAPVPSDNH

SEQ ID NO: 583 - protein, 318 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARPSRAGSSPWWKTCSASGPGWWRRCRLPWAPAPPLCPATIT

SEQ ID NO: 584 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSCFEPLVEDMQRQWAGLVEKVQAAVGTSAAPVPSDNH

SEQ ID NO: 585 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFGPLVEDMQRQWAGLVEKVQAAVGTSAAPVPSDNH

SEQ ID NO: 586 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFDPLVEDMQRQWAGLVEKVQAAVGTSAAPVPSDNH

SEQ ID NO: 587 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPMVEDMQRQWAGLVEKVQAAVGTSAAPVPSDNH

SEQ ID NO: 588 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLMEDMQRQWAGLVEKVQAAVGTSAAPVPSDNH

SEQ ID NO: 589 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEAMQRQWAGLVEKVQAAVGTSAAPVPSDNH

SEQ ID NO: 590 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQCQWAGLVEKVQAAVGTSAAPVPSDNH

SEQ ID NO: 591 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQHQWAGLVEKVQAAVGTSAAPVPSDNH

SEQ ID NO: 592 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQCAGLVEKVQAAVGTSAAPVPSDNH

SEQ ID NO: 593 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWTGLVEKVQAAVGTSAAPVPSDNH

SEQ ID NO: 594 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWARLVEKVQAAVGTSAAPVPSDNH

SEQ ID NO: 595 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAWLVEKVQAAVGTSAAPVPSDNH

SEQ ID NO: 596 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGPVEKVQAAVGTSAAPVPSDNH

SEQ ID NO: 597 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVQKVQAAVGTSAAPVPSDNH

SEQ ID NO: 598 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVERVQAAVGTSAAPVPSDNH

SEQ ID NO: 599 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVENVQAAVGTSAAPVPSDNH

SEQ ID NO: 600 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKLQAAVGTSAAPVPSDNH

SEQ ID NO: 601 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQSAVGTSAAPVPSDNH

SEQ ID NO: 602 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAMGTSAAPVPSDNH

SEQ ID NO: 603 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGISAAPVPSDNH

SEQ ID NO: 604 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSTAPVPSDNH

SEQ ID NO: 605 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAAVPSDNH

SEQ ID NO: 606 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPRDNH

SEQ ID NO: 607 - protein, 355 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNHSTPKPAAMRPHATPCLLPPRSLQRETLSPPQPSSWGGP

SEQ ID NO: 608 - protein, 37 aa, Homo sapiens
MKVLWAALLVTFLAGCQAKVEQAVETEPEPELRQQTE

SEQ ID NO: 609 - protein, 97 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELE

SEQ ID NO: 610 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQTAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 611 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARMGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 612 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVRAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 613 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLCKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 614 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLPKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 615 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRERLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 616 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKPLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 617 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDPDDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 618 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAAAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 619 - protein, 269 aa, Homo sapiens
MISGASRKSWDPGNPWPPDWPITGRKMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRT

SEQ ID NO: 620 - protein, 269 aa, Homo sapiens
MSSEASRKSWDPGNPWPPDWPITGRKMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRT

SEQ ID NO: 621 - protein, 269 aa, Homo sapiens
MSSGTSRKSWDPGNPWPPDWPITGRKMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRT

SEQ ID NO: 622 - protein, 269 aa, Homo sapiens
MSSGAFRKSWDPGNPWPPDWPITGRKMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRT

SEQ ID NO: 623 - protein, 269 aa, Homo sapiens
MSSGASKKSWDPGNPWPPDWPITGRKMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRT

SEQ ID NO: 624 - protein, 269 aa, Homo sapiens
MSSGASRKSWDSGNPWPPDWPITGRKMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRT

SEQ ID NO: 625 - protein, 269 aa, Homo sapiens [sequence omitted]

SEQ ID NO: 626 - protein, 269 aa, Homo sapiens
MSSGASRKSWDPGNPWSPDWPITGRKMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRT

SEQ ID NO: 627 - protein, 269 aa, Homo sapiens
MSSGASRKSWDPGNPWPLDWPITGRKMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRT

SEQ ID NO: 628 - protein, 269 aa, Homo sapiens
MSSGASRKSWDPGNPWPPNWPITGRKMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRT

SEQ ID NO: 629 - protein, 269 aa, Homo sapiens
MSSGASRKSWDPGNPWPPDCPITGRKMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRT

SEQ ID NO: 630 - protein, 21 aa, Homo sapiens
MSSGASRKSWDPGNPWPPDWP

SEQ ID NO: 631 - protein, 269 aa, Homo sapiens
MSSGASRKSWDPGNPWPPDWPIKGRKMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRT

SEQ ID NO: 632 - protein, 269 aa, Homo sapiens
MSSGASRKSWDPGNPWPPDWPITDRKMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRT

SEQ ID NO: 633 - protein, 269 aa, Homo sapiens
MSSGASRKSWDPGNPWPPDWPITVRKMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRT

SEQ ID NO: 634 - protein, 25 aa, Homo sapiens
MSSGASRKSWDPGNPWPPDWPITGR

SEQ ID NO: 635 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTP

SEQ ID NO: 636 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTE

SEQ ID NO: 637 - protein, 266 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 638 - protein, 268 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNNTQ

SEQ ID NO: 639 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYSKKLNTQ

SEQ ID NO: 640 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSTLEEYTKKLNTQ

SEQ ID NO: 641 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFRSALEEYTKKLNTQ

SEQ ID NO: 642 - protein, 248 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRHPERSRGVH

SEQ ID NO: 643 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSLLSALEEYTKKLNTQ

SEQ ID NO: 644 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKDSFLSALEEYTKKLNTQ

SEQ ID NO: 645 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLVPVLESFKVSFLSALEEYTKKLNTQ

SEQ ID NO: 646 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGMLPVLESFKVSFLSALEEYTKKLNTQ

SEQ ID NO: 647 - protein, 266 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDRQGLLPVLESFKVSFLSALEEYTKKLNTQ

SEQ ID NO: 648 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDPRQGLLPVLESFKVSFLSALEEYTKKLNTQ

SEQ ID NO: 649 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALKDLRQGLLPVLESFKVSFLSALEEYTKKLNTQ

SEQ ID NO: 650 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKLALEDLRQGLLPVLESFKVSFLSALEEYTKKLNTQ

SEQ ID NO: 651 - protein, 266 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEAKPALEDLRQGLLPVLESFKVSFLSALEEYTKKLNTQ

SEQ ID NO: 652 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSDKAKPALEDLRQGLLPVLESFKVSFLSALEEYTKKLNTQ

SEQ ID NO: 653 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSKKAKPALEDLRQGLLPVLESFKVSFLSALEEYTKKLNTQ

SEQ ID NO: 654 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLREKAKPALEDLRQGLLPVLESFKVSFLSALEEYTKKLNTQ

SEQ ID NO: 655 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLGTLSEKAKPALEDLRQGLLPVLESFKVSFLSALEEYTKKLNTQ

SEQ ID NO: 656 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATKHLSTLSEKAKPALEDLRQGLLPVLESFKVSFLSALEEYTKKLNTQ

SEQ ID NO: 657 - protein, 221 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKAT

SEQ ID NO: 658 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKASEHLSTLSEKAKPALEDLRQGLLPVLESFKVSFLSALEEYTKKLNTQ

SEQ ID NO: 659 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKPTEHLSTLSEKAKPALEDLRQGLLPVLESFKVSFLSALEEYTKKLNTQ

SEQ ID NO: 660 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHGKATEHLSTLSEKAKPALEDLRQGLLPVLESFKVSFLSALEEYTKKLNTQ

SEQ ID NO: 661 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAQYHAKATEHLSTLSEKAKPALEDLRQGLLPVLESFKVSFLSALEEYTKKLNTQ

SEQ ID NO: 662 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARVAEYHAKATEHLSTLSEKAKPALEDLRQGLLPVLESFKVSFLSALEEYTKKLNTQ

SEQ ID NO: 663 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGASLAEYHAKATEHLSTLSEKAKPALEDLRQGLLPVLESFKVSFLSALEEYTKKLNTQ

SEQ ID NO: 664 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGAILAEYHAKATEHLSTLSEKAKPALEDLRQGLLPVLESFKVSFLSALEEYTKKLNTQ

SEQ ID NO: 665 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENRGARLAEYHAKATEHLSTLSEKAKPALEDLRQGLLPVLESFKVSFLSALEEYTKKLNTQ

SEQ ID NO: 666 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKEYGGARLAEYHAKATEHLSTLSEKAKPALEDLRQGLLPVLESFKVSFLSALEEYTKKLNTQ

SEQ ID NO: 667 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKDNGGARLAEYHAKATEHLSTLSEKAKPALEDLRQGLLPVLESFKVSFLSALEEYTKKLNTQ

SEQ ID NO: 668 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALRENGGARLAEYHAKATEHLSTLSEKAKPALEDLRQGLLPVLESFKVSFLSALEEYTKKLNTQ

SEQ ID NO: 669 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEAVKENGGARLAEYHAKATEHLSTLSEKAKPALEDLRQGLLPVLESFKVSFLSALEEYTKKLNTQ

SEQ ID NO: 670 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEVLKENGGARLAEYHAKATEHLSTLSEKAKPALEDLRQGLLPVLESFKVSFLSALEEYTKKLNTQ

SEQ ID NO: 671 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAAHLEALKENGGARLAEYHAKATEHLSTLSEKAKPALEDLRQGLLPVLESFKVSFLSALEEYTKKLNTQ

SEQ ID NO: 672 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAASLEALKENGGARLAEYHAKATEHLSTLSEKAKPALEDLRQGLLPVLESFKVSFLSALEEYTKKLNTQ

SEQ ID NO: 673 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLTARLEALKENGGARLAEYHAKATEHLSTLSEKAKPALEDLRQGLLPVLESFKVSFLSALEEYTKKLNTQ

SEQ ID NO: 674 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQSLAARLEALKENGGARLAEYHAKATEHLSTLSEKAKPALEDLRQGLLPVLESFKVSFLSALEEYTKKLNTQ

SEQ ID NO: 675 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRPRLAARLEALKENGGARLAEYHAKATEHLSTLSEKAKPALEDLRQGLLPVLESFKVSFLSALEEYTKKLNTQ

SEQ ID NO: 676 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDKLRQRLAARLEALKENGGARLAEYHAKATEHLSTLSEKAKPALEDLRQGLLPVLESFKVSFLSALEEYTKKLNTQ

SEQ ID NO: 677 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSEELRQRLAARLEALKENGGARLAEYHAKATEHLSTLSEKAKPALEDLRQGLLPVLESFKVSFLSALEEYTKKLNTQ

SEQ ID NO: 678 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSNELRQRLAARLEALKENGGARLAEYHAKATEHLSTLSEKAKPALEDLRQGLLPVLESFKVSFLSALEEYTKKLNTQ

SEQ ID NO: 679 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPCSDELRQRLAARLEALKENGGARLAEYHAKATEHLSTLSEKAKPALEDLRQGLLPVLESFKVSFLSALEEYTKKLNTQ

SEQ ID NO: 680 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLARYSDELRQRLAARLEALKENGGARLAEYHAKATEHLSTLSEKAKPALEDLRQGLLPVLESFKVSFLSALEEYTKKLNTQ

SEQ ID NO: 681 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLGPYSDELRQRLAARLEALKENGGARLAEYHAKATEHLSTLSEKAKPALEDLRQGLLPVLESFKVSFLSALEEYTKKLNTQ

SEQ ID NO: 682 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLVPYSDELRQRLAARLEALKENGGARLAEYHAKATEHLSTLSEKAKPALEDLRQGLLPVLESFKVSFLSALEEYTKKLNTQ

SEQ ID NO: 683 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLSPYSDELRQRLAARLEALKENGGARLAEYHAKATEHLSTLSEKAKPALEDLRQGLLPVLESFKVSFLSALEEYTKKLNTQ

SEQ ID NO: 684 - protein, 231 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTPMWTRCARIWPPTATSCASAWPRALRLSRRTAAPDWPSTTPRPPSI

SEQ ID NO: 685 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALLTHLAPYSDELRQRLAARLEALKENGGARLAEYHAKATEHLSTLSEKAKPALEDLRQGLLPVLESFKVSFLSALEEYTKKLNTQ

SEQ ID NO: 686 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDVLRTHLAPYSDELRQRLAARLEALKENGGARLAEYHAKATEHLSTLSEKAKPALEDLRQGLLPVLESFKVSFLSALEEYTKKLNTQ

SEQ ID NO: 687 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDELRTHLAPYSDELRQRLAARLEALKENGGARLAEYHAKATEHLSTLSEKAKPALEDLRQGLLPVLESFKVSFLSALEEYTKKLNTQ

SEQ ID NO: 688 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDTLRTHLAPYSDELRQRLAARLEALKENGGARLAEYHAKATEHLSTLSEKAKPALEDLRQGLLPVLESFKVSFLSALEEYTKKLNTQ

SEQ ID NO: 689 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRALAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 690 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRVRAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 691 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRSRAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 692 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRTRAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 693 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEVMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 694 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEKMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 695 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGKEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 696 - protein, 169 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLG

SEQ ID NO: 697 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPPGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 698 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPVGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 699 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLRPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 700 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKRSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 701 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQENLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 702 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQGKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 703 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQKKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 704 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELPEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 705 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHDLQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 706 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHKLQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 707 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHQLQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 708 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLQELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 709 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARRKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 710 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGALQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 711 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGVRQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 712 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGTRQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 713 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQKGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 714 - protein, 150 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAEL

SEQ ID NO: 715 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAEIQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 716 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLHAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 717 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVKPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 718 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQRVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 719 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYCQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 720 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYSQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 721 - protein, 138 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMEL

SEQ ID NO: 722 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELHRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 723 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEETELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 724 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQAEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 725 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWEEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 726 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKCQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 727 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKLQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 728 - protein, 266 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 729 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKMWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 730 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQRKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 731 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFPKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 732 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDNFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 733 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLGDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 734 - protein, 123 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQP

SEQ ID NO: 735 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQSYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 736 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQTYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 737 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKAQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 738 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKVKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 739 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKELEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 740 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEISKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 741 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLSQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 742 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETDGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 743 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETKGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 744 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEEETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 745 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLGKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 746 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNPEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 747 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWNNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 748 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEYWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 749 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVIQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 750 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQFGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 751 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREHLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 752 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSNLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 753 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTYSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 754 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTISKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 755 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVISTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 756 - protein, 266 aa, Homo sapiens
MKAAVLTLAVLFLTGSQARHFWQQDEPPQSPWDRVKDLATVYVDVLKDSGRDYVSQFEGSALGKQLNLKLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 757 - protein, 267 aa, Homo sapiens
MKAAVLTLAVLFLTGSQARHFWQQDEPPQSPWDRVKDLATVYVDVLKDSGRDYVSQFEGSALGKQLNLKLVDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 758 - protein, 267 aa, Homo sapiens
MKAAVLTLAVLFLTGSQARHFWQQDEPPQSPWDRVKDLATVYVDVLKDSGRDYVSQFEGSALGKQLKLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 759 - protein, 62 aa, Homo sapiens
MKAAVLTLAVLFLTGSQARHFWQQDEPPQSPWDRVKDLATVYVDVLKDSGRDYVSQFEGSAL

SEQ ID NO: 760 - protein, 267 aa, Homo sapiens
MKAAVLTLAVLFLTGSQARHFWQQDEPPQSPWDRVKDLATVYVDVLKDSGRDYVSQFEGSAFGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 761 - protein, 267 aa, Homo sapiens
MKAAVLTLAVLFLTGSQARHFWQQDEPPQSPWDRVKDLATVYVDVLKDSGRDYVSQFEGSTLGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 762 - protein, 267 aa, Homo sapiens
MKAAVLTLAVLFLTGSQARHFWQQDEPPQSPWDRVKDLATVYVDVLKDSGRDYVSQFEGPALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 763 - protein, 267 aa, Homo sapiens
MKAAVLTLAVLFLTGSQARHFWQQDEPPQSPWDRVKDLATVYVDVLKDSGRDYVSQFEGAALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 764 - protein, 267 aa, Homo sapiens
MKAAVLTLAVLFLTGSQARHFWQQDEPPQSPWDRVKDLATVYVDVLKDSGRDYVSQFEVSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 765 - protein, 267 aa, Homo sapiens
MKAAVLTLAVLFLTGSQARHFWQQDEPPQSPWDRVKDLATVYVDVLKDSGRDYVSQFEASALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 766 - protein, 52 aa, Homo sapiens
MKAAVLTLAVLFLTGSQARHFWQQDEPPQSPWDRVKDLATVYVDVLKDSGRD

SEQ ID NO: 767 - protein, 267 aa, Homo sapiens
MKAAVLTLAVLFLTGSQARHFWQQDEPPQSPWDRVKDLATVYVDVLKDSGRDCVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 768 - protein, 267 aa, Homo sapiens
MKAAVLTLAVLFLTGSQARHFWQQDEPPQSPWDRVKDLATVYVDVLKDSVRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 769 - protein, 267 aa, Homo sapiens
MKAAVLTLAVLFLTGSQARHFWQQDEPPQSPWDRVKDLATVYVDVLKDSSRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 770 - protein, 267 aa, Homo sapiens
MKAAVLTLAVLFLTGSQARHFWQQDEPPQSPWDRVKDLATVYVDVLKDNGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 771 - protein, 267 aa, Homo sapiens
MKAAVLTLAVLFLTGSQARHFWQQDEPPQSPWDRVKDLATVYVDVLKDIGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 772 - protein, 267 aa, Homo sapiens
MKAAVLTLAVLFLTGSQARHFWQQDEPPQSPWDRVKDLATVYVDVLKDRGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 773 - protein, 267 aa, Homo sapiens
MKAAVLTLAVLFLTGSQARHFWQQDEPPQSPWDRVKDLATVYVEVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 774 - protein, 267 aa, Homo sapiens
MKAAVLTLAVLFLTGSQARHFWQQDEPPQSPWDRVKDLATVYLDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 775 - protein, 267 aa, Homo sapiens
MKAAVLTLAVLFLTGSQARHFWQQDEPPQSPWDRVKDLATVYMDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 776 - protein, 267 aa, Homo sapiens
MKAAVLTLAVLFLTGSQARHFWQQDEPPQSPWDRVKDLATLYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 777 - protein, 267 aa, Homo sapiens
MKAAVLTLAVLFLTGSQARHFWQQDEPPQSPWDRVKDLAAVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 778 - protein, 267 aa, Homo sapiens
MKAAVLTLAVLFLTGSQARHFWQQDEPPQSPWDRVKDLVTVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 779 - protein, 267 aa, Homo sapiens
MKAAVLTLAVLFLTGSQARHFWQQDEPPQSPWDLVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 780 - protein, 267 aa, Homo sapiens
MKAAVLTLAVLFLTGSQARHFWQQDEPPQSPWDQVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 781 - protein, 267 aa, Homo sapiens
MKAAVLTLAVLFLTGSQARHFWQQDEPPQSLWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 782 - protein, 267 aa, Homo sapiens
MKAAVLTLAVLFLTGSQARHFWQQDEPPQSAWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 783 - protein, 267 aa, Homo sapiens
MKAAVLTLAVLFLTGSQARHFWQQDEPPQGPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 784 - protein, 267 aa, Homo sapiens
MKAAVLTLAVLFLTGSQARHFWQQDEPPQRPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 785 - protein, 267 aa, Homo sapiens
MKAAVLTLAVLFLTGSQARHFWQQDEPPHSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 786 - protein, 267 aa, Homo sapiens
MKAAVLTLAVLFLTGSQARHFWQQDEPPKSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 787 - protein, 267 aa, Homo sapiens
MKAAVLTLAVLFLTGSQARHFWQQDEPRQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 788 - protein, 267 aa, Homo sapiens
MKAAVLTLAVLFLTGSQARHFWQQDEPSQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 789 - protein, 267 aa, Homo sapiens
MKAAVLTLAVLFLTGSQARHFWQQDETPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 790 - protein, 267 aa, Homo sapiens
MKAAVLTLAVLFLTGSQARHFWQQDESPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 791 - protein, 267 aa, Homo sapiens
MKAAVLTLAVLFLTGSQAPHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 792 - protein, 267 aa, Homo sapiens
MKAAVLTLAVLFLTGSQAWHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 793 - protein, 267 aa, Homo sapiens
MKAAVLTLAVLFLMGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 794 - protein, 267 aa, Homo sapiens
MKAAVLTLAMLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 795 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSMLSEKAKPALEDLRQGLLPVLESFKVSFLSALEEYTKKLNTQ

SEQ ID NO: 796 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENSGARLAEYHAKATEHLSTLSEKAKPALEDLRQGLLPVLESFKVSFLSALEEYTKKLNTQ

SEQ ID NO: 797 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARPEALKENGGARLAEYHAKATEHLSTLSEKAKPALEDLRQGLLPVLESFKVSFLSALEEYTKKLNTQ

SEQ ID NO: 798 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLPARLEALKENGGARLAEYHAKATEHLSTLSEKAKPALEDLRQGLLPVLESFKVSFLSALEEYTKKLNTQ

SEQ ID NO: 799 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRSAARLEALKENGGARLAEYHAKATEHLSTLSEKAKPALEDLRQGLLPVLESFKVSFLSALEEYTKKLNTQ

SEQ ID NO: 800 - protein, 267 aa, Homo sapiens; identical to SEQ ID NO: 374

SEQ ID NO: 801 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVGALRTHLAPYSDELRQRLAARLEALKENGGARLAEYHAKATEHLSTLSEKAKPALEDLRQGLLPVLESFKVSFLSALEEYTKKLNTQ

SEQ ID NO: 802 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHEDALRTHLAPYSDELRQRLAARLEALKENGGARLAEYHAKATEHLSTLSEKAKPALEDLRQGLLPVLESFKVSFLSALEEYTKKLNTQ

SEQ ID NO: 803 - protein, 202 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARCPCGRAAHASGPLQRRAAPALGRAP

SEQ ID NO: 804 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRAPAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 805 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMPDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 806 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSRLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 807 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQGGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 808 - protein, 130 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQK

SEQ ID NO: 809 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDPEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 810 - protein, 107 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLR

SEQ ID NO: 811 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQELWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 812 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKRREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 813 - protein, 267 aa, Homo sapiens
MKAAVLTLAVLFLTGSQARHFWQQDEPPQSPWDRVKDLATVYVDVLKDSGRDYVSQFEGSALGKQLNLKLLDNRDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 814 - protein, 55 aa, Homo sapiens
MKAAVLTLAVLFLTGSQARHFWQQDEPPQSPWDRVKDLATVYVDVLKDSGRDYVS

SEQ ID NO: 815 - protein, 267 aa, Homo sapiens
MKAAVLTLAVLFLTGSQARHFWQQDEPPQSPWDRVKDLATVYVDVLKDSRRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 816 - protein, 57 aa, Homo sapiens
MKAAVLTLAVLFLTGSQARHFWQQDEPPAEPLGSSEGPGHCVRGCAQRQRQRLCVPV

SEQ ID NO: 817 - protein, 267 aa, Homo sapiens
MKAAVLTLAVLFLTGSQARHFWQQDEHPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 818 - protein, 267 aa, Homo sapiens
MKAAVLTLAVLFLTGSQARHFWQQDERPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 819 - protein, 22 aa, Homo sapiens
MKAAVLTLAVLFLTGSQARHFW

SEQ ID NO: 820 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHPKATEHLSTLSEKAKPALEDLRQGLLPVLESFKVSFLSALEEYTKKLNTQ

SEQ ID NO: 821 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGAGLAEYHAKATEHLSTLSEKAKPALEDLRQGLLPVLESFKVSFLSALEEYTKKLNTQ

SEQ ID NO: 822 - protein, 245 aa, Homo sapiens
MSGGARGFCMLKAPHSARPFFSSRSPTALQDESCGADLGRALPDRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 823 - protein, 245 aa, Homo sapiens
MSGGARGFCMLKAPHSARPFFSSRSPTALQDESCGADLGRALPDSLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 824 - protein, 245 aa, Homo sapiens
MSGGARGFCMLKAPHSARPFFSSRSPTALQDESCGADLGCALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 825 - protein, 245 aa, Homo sapiens
MSGGARGFCMLKAPHSARPFFSSRSPTALQDESCSADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 826 - protein, 245 aa, Homo sapiens
MSGGARGFCMLKAPHSARPFFSSRSPTALQDESRGADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 827 - protein, 245 aa, Homo sapiens
MSGGARGFCMLKAPHSARPFFSSRSPTSLQDESCGADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 828 - protein, 245 aa, Homo sapiens
MSGGARGFCMLKAPHSARPFFSSRSPMALQDESCGADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 829 - protein, 26 aa, Homo sapiens
MSGGARGFCMLKAPHSARPFFSSRSP

SEQ ID NO: 830 - protein, 252 aa, Homo sapiens
MSGGARGFCMLKAPHSARPFFSSARPFFSSRSPTALQDESCGADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 831 - protein, 244 aa, Homo sapiens
MSGGARGFCMLKAPHSARPFSSRSPTALQDESCGADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 832 - protein, 245 aa, Homo sapiens
MSGGARGFCMLKAPHSARPFFPSRSPTALQDESCGADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 833 - protein, 245 aa, Homo sapiens
MSGGARGFCMLKAPHSARPFLSSRSPTALQDESCGADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 834 - protein, 245 aa, Homo sapiens
MSGGARGFCMLKAPHSARPFSSSRSPTALQDESCGADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 835 - protein, 245 aa, Homo sapiens
MSGGARGFCMLKAPHSARPLFSSRSPTALQDESCGADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 836 - protein, 19 aa, Homo sapiens
MSGGARGFCMLKAPHSARP

SEQ ID NO: 837 - protein, 245 aa, Homo sapiens
MSGGARGFCMLKAPLSARPFFSSRSPTALQDESCGADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 838 - protein, 245 aa, Homo sapiens
MSGGARGFCMLKASHSARPFFSSRSPTALQDESCGADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 839 - protein, 245 aa, Homo sapiens
MSGGARSFCMLKAPHSARPFFSSRSPTALQDESCGADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 840 - protein, 245 aa, Homo sapiens
MSGGAQGFCMLKAPHSARPFFSSRSPTALQDESCGADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 841 - protein, 245 aa, Homo sapiens
MSGGAWGFCMLKAPHSARPFFSSRSPTALQDESCGADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 842 - protein, 245 aa, Homo sapiens
MSGRARGFCMLKAPHSARPFFSSRSPTALQDESCGADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 843 - protein, 245 aa, Homo sapiens
MSAGARGFCMLKAPHSARPFFSSRSPTALQDESCGADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 844 - protein, 245 aa, Homo sapiens
MSRGARGFCMLKAPHSARPFFSSRSPTALQDESCGADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 845 - protein, 245 aa, Homo sapiens
ISGGARGFCMLKAPHSARPFFSSRSPTALQDESCGADLGRALPDGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 846 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAAQARLKSWFEPLVEDMQRQWAGLVEKVQAAVGTSAAPVPSDNH

SEQ ID NO: 847 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSRFEPLVEDMQRQWAGLVEKVQAAVGTSAAPVPSDNH

SEQ ID NO: 848 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLAEDMQRQWAGLVEKVQAAVGTSAAPVPSDNH

SEQ ID NO: 849 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGQVEKVQAAVGTSAAPVPSDNH

SEQ ID NO: 850 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAEGTSAAPVPSDNH

SEQ ID NO: 851 - protein, 25 aa, synthetic construct
RLASHLRKLRKRLLRDADDLQKRLA

SEQ ID NO: 852 - protein, 6 aa, synthetic construct
HHHHHH

SEQ ID NO: 853 - protein, 8 aa, synthetic construct
WSHPQFEK

SEQ ID NO: 854 - protein, 8 aa, synthetic construct
DYKDDDDK

SEQ ID NO: 855 - protein, 317 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAAAVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 856 - protein, 317 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKCLAVYQAGAREGAERGLSAIRERLGPLVEQGRVRAAAVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 857 - protein, 317 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVRGRLVQYRGEVQAMLGQSTEELRVRLASHLRKLRKRLLRDADDLQKRLAVYQAGAREGAERGLSAIRERLGPLVEQGRVRAAAVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 858 - protein, 11 aa, synthetic construct
VLMEDTMTEVK

SEQ ID NO: 859 - protein, 11 aa, synthetic construct
AYKKELEEQLG

SEQ ID NO: 860 - protein, 11 aa, synthetic construct
PVAEETRARLA

SEQ ID NO: 861 - protein, 11 aa, synthetic construct
KEVQAAQARLG

SEQ ID NO: 862 - protein, 11 aa, synthetic construct
ADMEDLRNRLG

SEQ ID NO: 863 - protein, 11 aa, synthetic construct
QYRNEVNTMLG

SEQ ID NO: 864 - protein, 11 aa, synthetic construct
QSTEELRSRLS

SEQ ID NO: 865 - protein, 11 aa, synthetic construct
THLRKMRKRLM

SEQ ID NO: 866 - protein, 11 aa, synthetic construct
VYKAGAQEGAE

SEQ ID NO: 867 - protein, 11 aa, synthetic construct
RGVSAIRERLG

SEQ ID NO: 868 - protein, 11 aa, synthetic construct
DRLEEVREQME

SEQ ID NO: 869 - protein, 11 aa, synthetic construct
SKMEEQTQQIR

SEQ ID NO: 870 - protein, 11 aa, synthetic construct
LQAEIFQARIK

SEQ ID NO: 871 - protein, 11 aa, synthetic construct
NLMEKIQASVA

SEQ ID NO: 872 - protein, 53 aa, synthetic construct
EGELEVTDQLPGQSDQPWEQALNRFWDYLRWVQTLSDQVQEELQSSQVTQELT

SEQ ID NO: 873 - protein, 14 aa, synthetic construct
TNSIASTTVPLENQ

SEQ ID NO: 874 - protein, 4 aa, synthetic construct
GWFE

SEQ ID NO: 875 - protein, 44 aa, synthetic construct
PLVEQGRQRTANLGAGAAQPLRDRAQALSDRIRGRLEEVGNQAR

SEQ ID NO: 876 - protein, 292 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQEAAKLEEQAQQIRLQAEAFQARLKPLVEDMQRQWAGLVEKVQAAVGTSAAPVPSDNH

SEQ ID NO: 877 - protein, 292 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQEAAKLEEQAQQIRLQAEAFQARLKPLVEDMQRQWAGLVEKVQAAVGTSAAPVPSDNH

SEQ ID NO: 878 - protein, 248 aa, synthetic construct; identical to SEQ ID NO: 277

SEQ ID NO: 879 - protein, 248 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGDRLDEVKEQEAAKLEEQAQQIRLQAEAFQARLKPLVEDMQRQWAGLVEKVQAAVGTSAAPVPSDNH

SEQ ID NO: 880 - protein, 248 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGDRLDEVKEQEAAKLEEQAQQIRLQAEAFQARLKPLVEDMQRQWAGLVEKVQAAVGTSAAPVPSDNH

SEQ ID NO: 881 - protein, 248 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGDRLDEVKEQEAAKLEEQAQQIRLQAEAFQARLKPLVEDMQRQWAGQVEKVQAAVGTSAAPVPSDNH